SEQUENCE LISTING (322 sequences)

SEQ ID NO: 1 - DNA, 290 bp, Artificial Sequence
  Description of Artificial Sequence Synthetic polynucleotide
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 2 - DNA, 173 bp, Artificial Sequence
  Description of Artificial Sequence Synthetic polynucleotide
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 3 - DNA, 305 bp, Artificial Sequence
  Description of Artificial Sequence Synthetic polynucleotide
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 4 - DNA, 180 bp, Artificial Sequence
  Description of Artificial Sequence Synthetic polynucleotide
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 5 - DNA, 199 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 6 - DNA, 117 bp, Artificial Sequence
  Description of Artificial Sequence Synthetic polynucleotide
atccccatcactcttgatggagatcaattccccaagctgctagagcgttaccttgcccttaaacattagcaatgtcgatttatcagagggccgacaggctcccacaggagaaaaccg

SEQ ID NO: 7 - DNA, 108 bp, Artificial Sequence
  Description of Artificial Sequence Synthetic polynucleotide
ctcttgatcgttatcaattcccacgctgtttcagagcgttaccttgcccttaaacattagcaatgtcgatttatcagagggccgacaggctcccacaggagaaaaccg

SEQ ID NO: 8 - DNA, 290 bp, Artificial Sequence; identical to SEQ ID NO: 1
  Description of Artificial Sequence Synthetic polynucleotide

SEQ ID NO: 9 - DNA, 433 bp, Artificial Sequence
  Description of Artificial Sequence Synthetic polynucleotide
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 10 - DNA, 290 bp, Artificial Sequence
  Description of Artificial Sequence Synthetic polynucleotide
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 11 - DNA, 173 bp, Artificial Sequence; identical to SEQ ID NO: 2
  Description of Artificial Sequence Synthetic polynucleotide

SEQ ID NO: 12 - DNA, 305 bp, Artificial Sequence; identical to SEQ ID NO: 3
  Description of Artificial Sequence Synthetic polynucleotide

SEQ ID NO: 13 - DNA, 180 bp, Artificial Sequence; identical to SEQ ID NO: 4
  Description of Artificial Sequence Synthetic polynucleotide

SEQ ID NO: 14 - DNA, 199 bp, Artificial Sequence; identical to SEQ ID NO: 5
  Description of Artificial Sequence Synthetic polynucleotide

SEQ ID NO: 15 - DNA, 207 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 16 - DNA, 390 bp, Artificial Sequence
  Description of Artificial Sequence Synthetic polynucleotide
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 17 - DNA, 200 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 18 - DNA, 355 bp, Artificial Sequence
  Description of Artificial Sequence Synthetic polynucleotide
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 19 - DNA, 228 bp, Artificial Sequence
  Description of Artificial Sequence Synthetic polynucleotide
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 20 - DNA, 334 bp, Artificial Sequence
  Description of Artificial Sequence Synthetic polynucleotide
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 21 - DNA, 134 bp, Artificial Sequence
  Description of Artificial Sequence Synthetic polynucleotide
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 22 - DNA, 6734 bp, Artificial Sequence
  Description of Artificial Sequence Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaataaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtagtactgatcaaaaaggttagcctcaagagggtcataaaaatgtcagagcagaaagtagctctggttaccggtgcgttaggtggtatcggaagtgagatctgccgccagcttgtgaccgccgggtacaagattatcgccaccgttgttccacgcgaagaagaccgcgaaaaacaatggttgcaaagtgaggggtttcaagactctgatgtgcgtttcgtattaacagatttaaacaatcacgaagctgcgacagcggcaattcaagaagcgattgccgccgaaggacgcgttgatgtattggtcaacaacgcggggatcacgcgcgatgctacatttaagaaaatgtcctatgagcaatggtcccaagtcatcgacacgaatttaaagactctttttaccgtgacccagccagtatttaataaaatgcttgaacagaagtctggccgcatcgtaaacattagctctgtcaatggtttaaaagggcaatttggtcaagccaactactcggcctcgaaagcagggattatcgggtttactaaagcattggcgcaggagggtgctcgctcgaacatttgcgtcaatgtcgttgctcctggttacacagcgacacccatggtcacagcaatgcgcgaggatgtaattaagtcaatcgaagctcaaattcccctgcaacgtctggcagcaccggcggagattgcggcagcggttatgtatttggtgagtgaacacggtgcatacgtgacgggcgaaactttgagtatcaacggcgggctgtacatgcactaaaggtgcttttagtctagcgctagagcaggtaccatattaatgaatccaaattcctttcagtttaaagagaatatcttacagtttttcagcgtgcacgacgatatttggaaaaaactgcaggaattttactatggacaatcgcccatcaatgaagcgttggcgcagttaaataaggaagacatgagtttattcttcgaggcgttatcaaaaaaccctgctcgtatgatggagatgcagtggtcctggtggcaagggcagattcaaatttaccagaacgtgttaatgcgtagtgtagccaaggacgtagccccctttatccagccagagtccggagatcgtcgcttcaactcgccactttggcaagaacatccaaattttgatttactgagtcaatcctacttgttgttttctcagttggttcaaaatatggtggatgtcgttgaaggagtacctgataaggtccgctatcgcatccatttctttacacgtcagatgatcaatgcgttgtctccttctaatttcctgtggacgaaccctgaagtaattcaacagacggtcgctgaacagggtgagaatttagtacgcgggatgcaagtatttcacgatgatgtaatgaattcgggtaaatatttgagcatccgtatggtaaatagcgacagtttctctcttggcaaggacttggcgtatacgccaggagccgtagttttcgagaacgacatctttcagcttcttcaatacgaagccacaaccgagaacgtatatcaaacccctattcttgtcgtacctcccttcatcaacaagtactacgtgctggacctgcgcgaacagaatagcttggttaattggctgcgccaacaaggacatacggtgtttttgatgtcgtggcgtaaccccaacgcagagcagaaggagcttaccttcgctgacttaattacccaaggatcggtagaagcattacgtgttatcgaagaaatcacgggagagaaagaagctaactgtattggatattgcatcggtggtacacttctggctgctacccaggcatattatgtagctaaacgcctgaaaaatcacgtaaagtcagcgacttatatggcgacgattattgattttgagaaccccggctcattgggtgttttcattaatgagccggtcgtaagtggacttgaaaaccttaataatcaacttggttacttcgacgggcgtcaacttgcagtgacattttcgttgttgcgcgaaaacaccttgtattggaattattacatcgataattacttgaagggtaaggaaccgtccgactttgacatcttatactggaactcggatggtacgaatatcccagcaaagattcacaatttcctgttacgtaacctttatcttaacaacgaacttatttctccaaatgccgtcaaagttaatggtgtgggtttaaacctttcgcgcgtgaagactccatcattcttcattgctacgcaggaggaccatatcgcattgtgggatacctgttttcgcggcgcggattacctggggggtgagagcacacttgtgcttggggaaagcggacacgtcgccggcattgtcaacccgccttctcgtaacaagtatggttgttacacgaacgccgccaagtttgaaaataccaagcaatggcttgacggtgcagaatatcatcccgaaagctggtggttacgttggcaggcatgggtcacgccttatactggagagcaggttcctgcgcgtaatttgggaaacgcacagtaccccagtattgaagcggcccctgggcgttatgtgctggtaaacctgttttaacgctcacatacaagcaatctataattattcacggtataaatgaaagatgttgttatcgtagccgctaaacgcactgcgatcggttcctttctggggagtctggcttccctgagcgcccctcagttgggtcagacggctatccgcgcagttttggattctgcaaatgtgaaaccagaacaagtggaccaagtaattatggggaatgtgctgaccaccggcgttgggcaaaatcctgctcgtcaggcagcaatcgccgctgggattcctgtacaagttcccgccagcacgcttaatgtagtgtgtgggtccggattacgtgccgttcacctggcagctcaagccatccaatgcgatgaagccgatatcgtcgttgccggaggtcaagaatcaatgtcccagtctgctcattacatgcagcttcgcaatggccagaaaatgggtaacgcacagttagtcgattcaatggtggccgacggcttgaccgacgcgtataatcaataccagatgggtatcaccgcggagaatatcgtcgaaaaacttggtcttaatcgtgaagaacaagaccagcttgctctgacaagtcaacaacgtgctgcagcagcgcaggctgccggaaaattcaaggatgaaattgcggtcgtttcgattccccagcgcaaaggagagccggtcgtcttcgcggaagacgaatatatcaaggccaatacctcgttggaatccttgacgaaactgcgtccagcattcaaaaaagacggttctgttacagccggcaacgcatctggcattaatgatggggcagccgcggtcctgatgatgtccgccgacaaagcggctgaactgggcttaaagcctttagcacgcattaaaggttacgcgatgtcaggaattgagccggaaatcatgggactgggtcctgtagacgccgttaagaaaacccttaataaggctggttggtccttagaccaggtcgatctgatcgaggccaatgaggcttttgctgcccaagcactgggagtagccaaggagcttgggctggacctggacaaggtaaatgttaacggaggtgcgatcgcgctgggacacccgatcggggcttcgggttgtcgtatcttggtcacgttattacacgaaatgcagcgtcgtgatgcaaagaagggtatcgccacattgtgtgtgggaggtggaatgggggtggcgcttgccgttgagcgcgattaaggaggtcggataaggcgctcgcgccgcatccgacaccgtgcgcagatgcctgatgcgacgctgacgcgtcttatcatgcctcgctctcgagtcccgtcaagtcagacgatcgcacgccccatgtgaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgat

SEQ ID NO: 23 - DNA, 6114 bp, Artificial Sequence
  Description of Artificial Sequence Synthetic polynucleotide
taattcctaatttttgttgacactctatcattgatagagttattttaccactccctatcagtgatagagaaaagtgaataaggcgtaaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtagtactgatcaaaaaggttagcctcaagagggtcataaaaatgtcagagcagaaagtagctctggttaccggtgcgttaggtggtatcggaagtgagatctgccgccagcttgtgaccgccgggtacaagattatcgccaccgttgttccacgcgaagaagaccgcgaaaaacaatggttgcaaagtgaggggtttcaagactctgatgtgcgtttcgtattaacagatttaaacaatcacgaagctgcgacagcggcaattcaagaagcgattgccgccgaaggacgcgttgatgtattggtcaacaacgcggggatcacgcgcgatgctacatttaagaaaatgtcctatgagcaatggtcccaagtcatcgacacgaatttaaagactctttttaccgtgacccagccagtatttaataaaatgcttgaacagaagtctggccgcatcgtaaacattagctctgtcaatggtttaaaagggcaatttggtcaagccaactactcggcctcgaaagcagggattatcgggtttactaaagcattggcgcaggagggtgctcgctcgaacatttgcgtcaatgtcgttgctcctggttacacagcgacacccatggtcacagcaatgcgcgaggatgtaattaagtcaatcgaagctcaaattcccctgcaacgtctggcagcaccggcggagattgcggcagcggttatgtatttggtgagtgaacacggtgcatacgtgacgggcgaaactttgagtatcaacggcgggctgtacatgcactaaaggtgcttttagtctagcgctagagcaggtaccatattaatgaatccaaattcctttcagtttaaagagaatatcttacagtttttcagcgtgcacgacgatatttggaaaaaactgcaggaattttactatggacaatcgcccatcaatgaagcgttggcgcagttaaataaggaagacatgagtttattcttcgaggcgttatcaaaaaaccctgctcgtatgatggagatgcagtggtcctggtggcaagggcagattcaaatttaccagaacgtgttaatgcgtagtgtagccaaggacgtagccccctttatccagccagagtccggagatcgtcgcttcaactcgccactttggcaagaacatccaaattttgatttactgagtcaatcctacttgttgttttctcagttggttcaaaatatggtggatgtcgttgaaggagtacctgataaggtccgctatcgcatccatttctttacacgtcagatgatcaatgcgttgtctccttctaatttcctgtggacgaaccctgaagtaattcaacagacggtcgctgaacagggtgagaatttagtacgcgggatgcaagtatttcacgatgatgtaatgaattcgggtaaatatttgagcatccgtatggtaaatagcgacagtttctctcttggcaaggacttggcgtatacgccaggagccgtagttttcgagaacgacatctttcagcttcttcaatacgaagccacaaccgagaacgtatatcaaacccctattcttgtcgtacctcccttcatcaacaagtactacgtgctggacctgcgcgaacagaatagcttggttaattggctgcgccaacaaggacatacggtgtttttgatgtcgtggcgtaaccccaacgcagagcagaaggagcttaccttcgctgacttaattacccaaggatcggtagaagcattacgtgttatcgaagaaatcacgggagagaaagaagctaactgtattggatattgcatcggtggtacacttctggctgctacccaggcatattatgtagctaaacgcctgaaaaatcacgtaaagtcagcgacttatatggcgacgattattgattttgagaaccccggctcattgggtgttttcattaatgagccggtcgtaagtggacttgaaaaccttaataatcaacttggttacttcgacgggcgtcaacttgcagtgacattttcgttgttgcgcgaaaacaccttgtattggaattattacatcgataattacttgaagggtaaggaaccgtccgactttgacatcttatactggaactcggatggtacgaatatcccagcaaagattcacaatttcctgttacgtaacctttatcttaacaacgaacttatttctccaaatgccgtcaaagttaatggtgtgggtttaaacctttcgcgcgtgaagactccatcattcttcattgctacgcaggaggaccatatcgcattgtgggatacctgttttcgcggcgcggattacctggggggtgagagcacacttgtgcttggggaaagcggacacgtcgccggcattgtcaacccgccttctcgtaacaagtatggttgttacacgaacgccgccaagtttgaaaataccaagcaatggcttgacggtgcagaatatcatcccgaaagctggtggttacgttggcaggcatgggtcacgccttatactggagagcaggttcctgcgcgtaatttgggaaacgcacagtaccccagtattgaagcggcccctgggcgttatgtgctggtaaacctgttttaacgctcacatacaagcaatctataattattcacggtataaatgaaagatgttgttatcgtagccgctaaacgcactgcgatcggttcctttctggggagtctggcttccctgagcgcccctcagttgggtcagacggctatccgcgcagttttggattctgcaaatgtgaaaccagaacaagtggaccaagtaattatggggaatgtgctgaccaccggcgttgggcaaaatcctgctcgtcaggcagcaatcgccgctgggattcctgtacaagttcccgccagcacgcttaatgtagtgtgtgggtccggattacgtgccgttcacctggcagctcaagccatccaatgcgatgaagccgatatcgtcgttgccggaggtcaagaatcaatgtcccagtctgctcattacatgcagcttcgcaatggccagaaaatgggtaacgcacagttagtcgattcaatggtggccgacggcttgaccgacgcgtataatcaataccagatgggtatcaccgcggagaatatcgtcgaaaaacttggtcttaatcgtgaagaacaagaccagcttgctctgacaagtcaacaacgtgctgcagcagcgcaggctgccggaaaattcaaggatgaaattgcggtcgtttcgattccccagcgcaaaggagagccggtcgtcttcgcggaagacgaatatatcaaggccaatacctcgttggaatccttgacgaaactgcgtccagcattcaaaaaagacggttctgttacagccggcaacgcatctggcattaatgatggggcagccgcggtcctgatgatgtccgccgacaaagcggctgaactgggcttaaagcctttagcacgcattaaaggttacgcgatgtcaggaattgagccggaaatcatgggactgggtcctgtagacgccgttaagaaaacccttaataaggctggttggtccttagaccaggtcgatctgatcgaggccaatgaggcttttgctgcccaagcactgggagtagccaaggagcttgggctggacctggacaaggtaaatgttaacggaggtgcgatcgcgctgggacacccgatcggggcttcgggttgtcgtatcttggtcacgttattacacgaaatgcagcgtcgtgatgcaaagaagggtatcgccacattgtgtgtgggaggtggaatgggggtggcgcttgccgttgagcgcgattaaggaggtcggataaggcgctcgcgccgcatccgacaccgtgcgcagatgcctgatgcgacgctgacgcgtcttatcatgcctcgctctcgagtcccgtcaagtcagacgatcgcacgccccatgtgaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgat

SEQ ID NO: 24 - DNA, 5730 bp, Artificial Sequence
  Description of Artificial Sequence Synthetic polynucleotide
taaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtagtactgatcaaaaaggttagcctcaagagggtcataaaaatgtcagagcagaaagtagctctggttaccggtgcgttaggtggtatcggaagtgagatctgccgccagcttgtgaccgccgggtacaagattatcgccaccgttgttccacgcgaagaagaccgcgaaaaacaatggttgcaaagtgaggggtttcaagactctgatgtgcgtttcgtattaacagatttaaacaatcacgaagctgcgacagcggcaattcaagaagcgattgccgccgaaggacgcgttgatgtattggtcaacaacgcggggatcacgcgcgatgctacatttaagaaaatgtcctatgagcaatggtcccaagtcatcgacacgaatttaaagactctttttaccgtgacccagccagtatttaataaaatgcttgaacagaagtctggccgcatcgtaaacattagctctgtcaatggtttaaaagggcaatttggtcaagccaactactcggcctcgaaagcagggattatcgggtttactaaagcattggcgcaggagggtgctcgctcgaacatttgcgtcaatgtcgttgctcctggttacacagcgacacccatggtcacagcaatgcgcgaggatgtaattaagtcaatcgaagctcaaattcccctgcaacgtctggcagcaccggcggagattgcggcagcggttatgtatttggtgagtgaacacggtgcatacgtgacgggcgaaactttgagtatcaacggcgggctgtacatgcactaaaggtgcttttagtctagcgctagagcaggtaccatattaatgaatccaaattcctttcagtttaaagagaatatcttacagtttttcagcgtgcacgacgatatttggaaaaaactgcaggaattttactatggacaatcgcccatcaatgaagcgttggcgcagttaaataaggaagacatgagtttattcttcgaggcgttatcaaaaaaccctgctcgtatgatggagatgcagtggtcctggtggcaagggcagattcaaatttaccagaacgtgttaatgcgtagtgtagccaaggacgtagccccctttatccagccagagtccggagatcgtcgcttcaactcgccactttggcaagaacatccaaattttgatttactgagtcaatcctacttgttgttttctcagttggttcaaaatatggtggatgtcgttgaaggagtacctgataaggtccgctatcgcatccatttctttacacgtcagatgatcaatgcgttgtctccttctaatttcctgtggacgaaccctgaagtaattcaacagacggtcgctgaacagggtgagaatttagtacgcgggatgcaagtatttcacgatgatgtaatgaattcgggtaaatatttgagcatccgtatggtaaatagcgacagtttctctcttggcaaggacttggcgtatacgccaggagccgtagttttcgagaacgacatctttcagcttcttcaatacgaagccacaaccgagaacgtatatcaaacccctattcttgtcgtacctcccttcatcaacaagtactacgtgctggacctgcgcgaacagaatagcttggttaattggctgcgccaacaaggacatacggtgtttttgatgtcgtggcgtaaccccaacgcagagcagaaggagcttaccttcgctgacttaattacccaaggatcggtagaagcattacgtgttatcgaagaaatcacgggagagaaagaagctaactgtattggatattgcatcggtggtacacttctggctgctacccaggcatattatgtagctaaacgcctgaaaaatcacgtaaagtcagcgacttatatggcgacgattattgattttgagaaccccggctcattgggtgttttcattaatgagccggtcgtaagtggacttgaaaaccttaataatcaacttggttacttcgacgggcgtcaacttgcagtgacattttcgttgttgcgcgaaaacaccttgtattggaattattacatcgataattacttgaagggtaaggaaccgtccgactttgacatcttatactggaactcggatggtacgaatatcccagcaaagattcacaatttcctgttacgtaacctttatcttaacaacgaacttatttctccaaatgccgtcaaagttaatggtgtgggtttaaacctttcgcgcgtgaagactccatcattcttcattgctacgcaggaggaccatatcgcattgtgggatacctgttttcgcggcgcggattacctggggggtgagagcacacttgtgcttggggaaagcggacacgtcgccggcattgtcaacccgccttctcgtaacaagtatggttgttacacgaacgccgccaagtttgaaaataccaagcaatggcttgacggtgcagaatatcatcccgaaagctggtggttacgttggcaggcatgggtcacgccttatactggagagcaggttcctgcgcgtaatttgggaaacgcacagtaccccagtattgaagcggcccctgggcgttatgtgctggtaaacctgttttaacgctcacatacaagcaatctataattattcacggtataaatgaaagatgttgttatcgtagccgctaaacgcactgcgatcggttcctttctggggagtctggcttccctgagcgcccctcagttgggtcagacggctatccgcgcagttttggattctgcaaatgtgaaaccagaacaagtggaccaagtaattatggggaatgtgctgaccaccggcgttgggcaaaatcctgctcgtcaggcagcaatcgccgctgggattcctgtacaagttcccgccagcacgcttaatgtagtgtgtgggtccggattacgtgccgttcacctggcagctcaagccatccaatgcgatgaagccgatatcgtcgttgccggaggtcaagaatcaatgtcccagtctgctcattacatgcagcttcgcaatggccagaaaatgggtaacgcacagttagtcgattcaatggtggccgacggcttgaccgacgcgtataatcaataccagatgggtatcaccgcggagaatatcgtcgaaaaacttggtcttaatcgtgaagaacaagaccagcttgctctgacaagtcaacaacgtgctgcagcagcgcaggctgccggaaaattcaaggatgaaattgcggtcgtttcgattccccagcgcaaaggagagccggtcgtcttcgcggaagacgaatatatcaaggccaatacctcgttggaatccttgacgaaactgcgtccagcattcaaaaaagacggttctgttacagccggcaacgcatctggcattaatgatggggcagccgcggtcctgatgatgtccgccgacaaagcggctgaactgggcttaaagcctttagcacgcattaaaggttacgcgatgtcaggaattgagccggaaatcatgggactgggtcctgtagacgccgttaagaaaacccttaataaggctggttggtccttagaccaggtcgatctgatcgaggccaatgaggcttttgctgcccaagcactgggagtagccaaggagcttgggctggacctggacaaggtaaatgttaacggaggtgcgatcgcgctgggacacccgatcggggcttcgggttgtcgtatcttggtcacgttattacacgaaatgcagcgtcgtgatgcaaagaagggtatcgccacattgtgtgtgggaggtggaatgggggtggcgcttgccgttgagcgcgattaa

SEQ ID NO: 25 - DNA, 1887 bp, Artificial Sequence
  Description of Artificial Sequence Synthetic polynucleotide
atgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtag

SEQ ID NO: 26 - DNA, 747 bp, Artificial Sequence
  Description of Artificial Sequence Synthetic polynucleotide
atgtcagagcagaaagtagctctggttaccggtgcgttaggtggtatcggaagtgagatctgccgccagcttgtgaccgccgggtacaagattatcgccaccgttgttccacgcgaagaagaccgcgaaaaacaatggttgcaaagtgaggggtttcaagactctgatgtgcgtttcgtattaacagatttaaacaatcacgaagctgcgacagcggcaattcaagaagcgattgccgccgaaggacgcgttgatgtattggtcaacaacgcggggatcacgcgcgatgctacatttaagaaaatgtcctatgagcaatggtcccaagtcatcgacacgaatttaaagactctttttaccgtgacccagccagtatttaataaaatgcttgaacagaagtctggccgcatcgtaaacattagctctgtcaatggtttaaaagggcaatttggtcaagccaactactcggcctcgaaagcagggattatcgggtttactaaagcattggcgcaggagggtgctcgctcgaacatttgcgtcaatgtcgttgctcctggttacacagcgacacccatggtcacagcaatgcgcgaggatgtaattaagtcaatcgaagctcaaattcccctgcaacgtctggcagcaccggcggagattgcggcagcggttatgtatttggtgagtgaacacggtgcatacgtgacgggcgaaactttgagtatcaacggcgggctgtacatgcactaa

SEQ ID NO: 27 - DNA, 1773 bp, Artificial Sequence
  Description of Artificial Sequence Synthetic polynucleotide
atgaatccaaattcctttcagtttaaagagaatatcttacagtttttcagcgtgcacgacgatatttggaaaaaactgcaggaattttactatggacaatcgcccatcaatgaagcgttggcgcagttaaataaggaagacatgagtttattcttcgaggcgttatcaaaaaaccctgctcgtatgatggagatgcagtggtcctggtggcaagggcagattcaaatttaccagaacgtgttaatgcgtagtgtagccaaggacgtagccccctttatccagccagagtccggagatcgtcgcttcaactcgccactttggcaagaacatccaaattttgatttactgagtcaatcctacttgttgttttctcagttggttcaaaatatggtggatgtcgttgaaggagtacctgataaggtccgctatcgcatccatttctttacacgtcagatgatcaatgcgttgtctccttctaatttcctgtggacgaaccctgaagtaattcaacagacggtcgctgaacagggtgagaatttagtacgcgggatgcaagtatttcacgatgatgtaatgaattcgggtaaatatttgagcatccgtatggtaaatagcgacagtttctctcttggcaaggacttggcgtatacgccaggagccgtagttttcgagaacgacatctttcagcttcttcaatacgaagccacaaccgagaacgtatatcaaacccctattcttgtcgtacctcccttcatcaacaagtactacgtgctggacctgcgcgaacagaatagcttggttaattggctgcgccaacaaggacatacggtgtttttgatgtcgtggcgtaaccccaacgcagagcagaaggagcttaccttcgctgacttaattacccaaggatcggtagaagcattacgtgttatcgaagaaatcacgggagagaaagaagctaactgtattggatattgcatcggtggtacacttctggctgctacccaggcatattatgtagctaaacgcctgaaaaatcacgtaaagtcagcgacttatatggcgacgattattgattttgagaaccccggctcattgggtgttttcattaatgagccggtcgtaagtggacttgaaaaccttaataatcaacttggttacttcgacgggcgtcaacttgcagtgacattttcgttgttgcgcgaaaacaccttgtattggaattattacatcgataattacttgaagggtaaggaaccgtccgactttgacatcttatactggaactcggatggtacgaatatcccagcaaagattcacaatttcctgttacgtaacctttatcttaacaacgaacttatttctccaaatgccgtcaaagttaatggtgtgggtttaaacctttcgcgcgtgaagactccatcattcttcattgctacgcaggaggaccatatcgcattgtgggatacctgttttcgcggcgcggattacctggggggtgagagcacacttgtgcttggggaaagcggacacgtcgccggcattgtcaacccgccttctcgtaacaagtatggttgttacacgaacgccgccaagtttgaaaataccaagcaatggcttgacggtgcagaatatcatcccgaaagctggtggttacgttggcaggcatgggtcacgccttatactggagagcaggttcctgcgcgtaatttgggaaacgcacagtaccccagtattgaagcggcccctgggcgttatgtgctggtaaacctgttttaa

SEQ ID NO: 28 - DNA, 1179 bp, Artificial Sequence
  Description of Artificial Sequence Synthetic polynucleotide
atgaaagatgttgttatcgtagccgctaaacgcactgcgatcggttcctttctggggagtctggcttccctgagcgcccctcagttgggtcagacggctatccgcgcagttttggattctgcaaatgtgaaaccagaacaagtggaccaagtaattatggggaatgtgctgaccaccggcgttgggcaaaatcctgctcgtcaggcagcaatcgccgctgggattcctgtacaagttcccgccagcacgcttaatgtagtgtgtgggtccggattacgtgccgttcacctggcagctcaagccatccaatgcgatgaagccgatatcgtcgttgccggaggtcaagaatcaatgtcccagtctgctcattacatgcagcttcgcaatggccagaaaatgggtaacgcacagttagtcgattcaatggtggccgacggcttgaccgacgcgtataatcaataccagatgggtatcaccgcggagaatatcgtcgaaaaacttggtcttaatcgtgaagaacaagaccagcttgctctgacaagtcaacaacgtgctgcagcagcgcaggctgccggaaaattcaaggatgaaattgcggtcgtttcgattccccagcgcaaaggagagccggtcgtcttcgcggaagacgaatatatcaaggccaatacctcgttggaatccttgacgaaactgcgtccagcattcaaaaaagacggttctgttacagccggcaacgcatctggcattaatgatggggcagccgcggtcctgatgatgtccgccgacaaagcggctgaactgggcttaaagcctttagcacgcattaaaggttacgcgatgtcaggaattgagccggaaatcatgggactgggtcctgtagacgccgttaagaaaacccttaataaggctggttggtccttagaccaggtcgatctgatcgaggccaatgaggcttttgctgcccaagcactgggagtagccaaggagcttgggctggacctggacaaggtaaatgttaacggaggtgcgatcgcgctgggacacccgatcggggcttcgggttgtcgtatcttggtcacgttattacacgaaatgcagcgtcgtgatgcaaagaagggtatcgccacattgtgtgtgggaggtggaatgggggtggcgcttgccgttgagcgcgattaa

SEQ ID NO: 29 - DNA, 5934 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccataccgggaaaccaccgcgcccagcttaattttatgagtaacgaagatttattcatttgcatcgaccacgtcgcgtatgcgtgcccggatgccgatgaagcttctaagtattaccaggaaacattcggttggcacgagttgcaccgcgaagagaatccagaacagggcgtggtggaaattatgatggcgcctgctgcgaaattgacggagcacatgactcaggtgcaagttatggcgcctttgaacgatgagagtacggtcgcgaagtggcttgcgaaacacaatgggcgtgctggattgcaccacatggcatggcgtgttgatgacatcgacgcagtgtccgcaacacttcgcgagcgcggtgtacagttgctttacgacgagccgaaactgggtacaggtgggaatcgtatcaacttcatgcatccgaaatctggtaaaggcgtgctgattgaactgacccagtaccccaagaattgataaaggtttttcctaagacgctagcgcataaggtccaccaaatgtcaagtacagaccaaggcacgaaccctgctgacacggatgatttaacgccaaccacattatccctggctggtgatttccctaaggctacggaagagcagtgggagcgcgaggttgaaaaggtgttgaaccgtgggcgcccacccgagaagcagttgacgtttgctgaatgtttaaaacgtcttactgtgcacacagtagatggcattgacatcgttccaatgtatcgcccgaaggatgcccctaagaaactggggtatccaggggttgctccctttacgcgtggcactacggttcgcaatggggatatggacgcttgggacgttcgcgccctgcacgaagaccctgatgaaaaattcacgcgcaaagctattctggaggggctggagcgcggcgtaacaagtttgcttcttcgtgtggaccctgatgcaatcgctcccgaacacttagacgaagtgttaagtgacgttttgctggaaatgaccaaggttgaggtgttttcccgctatgatcagggagctgcggctgaagctcttgtctcggtatatgagcgcagcgacaaaccggctaaagatttggccttaaatttgggactggacccaatcgcatttgctgcacttcagggcactgagccagacttgaccgtacttggtgattgggttcgtcgtttggctaaattcagcccagactcacgcgctgtaacaattgatgctaatatttatcacaacgccggtgcaggcgacgttgccgagctggcctgggcacttgcgaccggagcagagtacgtccgtgcgctggtagagcaaggattcaccgccacagaggcatttgataccattaacttccgtgtgacagcgacccatgatcaatttttaacgattgcccgccttcgtgcgttacgtgaagcgtgggctcgtatcggtgaggtattcggagtagatgaggataaacgtggagcgcgccagaatgctattacgtcctggcgtgaactgacacgcgaggatccctatgtgaacattttacgtggaagtattgccacgttctctgcgtccgttgggggcgcggagtctattaccactttgccattcacgcaggcattgggccttccagaggatgattttccattacgtatcgcacgtaatacaggaattgtcttagctgaggaggtaaacattgggcgtgtaaatgaccctgccggggggtcatactatgtggagagcttgactcgttctcttgcagatgcagcatggaaagagttccaagaggttgaaaagttgggtggtatgtctaaggccgtcatgaccgaacacgtcacgaaggttttagatgcttgcaacgcagagcgcgcgaagcgcttggccaaccgcaagcaacctattacggcagtttccgaatttccgatgattggcgcacgcagcattgagacgaaaccatttccggctgctccggcccgtaaagggctggcatggcaccgcgattccgaagtcttcgagcaacttatggaccgctccacgtcagtttcagagcgtccgaaagtatttttagcatgtcttgggacgcgccgcgattttggaggacgcgaaggattttcatctccggtttggcacattgccgggattgacacgcctcaagtagaaggtgggacgactgctgaaatcgtggaagcgttcaaaaaatctggggcccaagtcgccgatttatgttcgagtgccaaagtgtatgctcaacaaggcttagaggtggcaaaggctctgaaagcggctggggctaaggcgctgtatttgagcggagcatttaaggagttcggagacgatgcagcggaagccgaaaaacttatcgacggacgccttttcatgggcatggatgtcgttgacaccctgtcttccactttagatatccttggagtggcgaagtgataagcttaaaacaatttacatccggccggaacttactatgtctaccttacctcgctttgacagtgttgatttaggaaatgcgccggtcccagcagatgctgcacgtcgttttgaggaacttgcggcgaaagccgggaccggcgaagcctgggaaactgcggaacaaattccagtaggcacgttgtttaatgaagacgtatacaaggacatggattggcttgatacttacgctggcattcctcccttcgtccatggtccgtacgctactatgtatgcatttcgtccttggaccattcgccaatatgccggtttttcgactgcaaaggagtcaaacgcattttaccgtcgtaatttggctgcaggccagaaaggtcttagtgttgcttttgacttacccactcaccgcggttatgattccgacaacccccgcgtggccggagatgttggtatggccggtgtggctatcgattcgatttatgacatgcgtgagctgttcgccggcatcccattagatcagatgagcgtgtcgatgacaatgaacggtgctgtcttgccgattttggctctttatgtggttacggcggaggagcaaggcgtgaagccagaacaactggcgggtactattcaaaatgatattctgaaggaatttatggttcgtaatacatatatttacccgccgcaacctagtatgcgcattatcagcgagatttttgcatacacatcagcaaacatgccgaagtggaactccattagtatcagcggctatcatatgcaggaggctggagcgactgcggatatcgagatggcgtataccttagctgatggagttgattacatccgtgctggtgagtcagtaggacttaatgtggaccaatttgctccacgcctgtccttcttctggggcattggtatgaactttttcatggaggtagcgaagttacgcgctgcccgtatgctgtgggcgaagcttgtccaccagttcggcccgaaaaacccgaagagtatgtctctgcgcacgcactctcaaacatcgggttggtctttgacagctcaagacgtatataataacgttgtacgtacatgcatcgaagccatggctgctactcaaggccatactcaatcacttcatacaaattcgttggatgaagccattgcattgcctacggacttttcagcccgcattgcccgcaatactcaattatttctgcaacaagagagcgggacgactcgtgtgatcgacccttggtcaggttccgcatacgtcgaagagttgacttgggatttagctcgtaaagcctgggggcatattcaggaggttgagaaggtggggggcatggctaaggcaatcgagaaggggattccgaagatgcgcattgaggaggcagccgcccgtacccaagcacgtattgattcgggacgccagccattaattggggtcaataaataccgtctggagcacgaaccacccctggatgtgttgaaggtagacaatagcaccgtgttagctgagcaaaaggccaaacttgttaaattgcgcgcagaacgcgacccagaaaaggtcaaggctgctctggacaaaatcacttgggcggctggcaatcctgatgataaagaccctgatcgcaacttattaaagctgtgcattgatgcggggcgcgcgatggcaacggtaggagagatgagtgacgctttagagaaagtttttgggcgctacacagcgcaaattcgcactatttcaggagtatattcaaaagaagtcaaaaacactccggaagtcgaggaggctcgcgaactggtagaagagtttgagcaggccgaaggccgtcgcccacgtatcctgctggctaaaatggggcaggacggtcatgaccgtgggcaaaaggtcatcgcgactgcatacgccgatttgggatttgacgtggacgttggcccgttattccaaactcccgaggaaactgctcgccaagccgtcgaagccgatgtgcacgtagtgggggtgagctctctggcgggagggcatcttacgcttgtgcctgcgcttcgcaaagagctggacaagttgggtcgtccagatattctgattaccgtaggaggggttattcccgagcaggacttcgatgagcttcgtaaggatggcgctgttgaaatctacacaccggggacggtcattccagaatcggctatctctttagttaaaaaattgcgcgcctccctggatgcttgataaggagctcggtaccaaattccagaaaagagacgctttcgagcgtcttttttcgttttggtccgcgcaataaaaaagcccccggaaggtgatcttccgggggctttctcatgcgtt

SEQ ID NO: 30 - DNA, 4968 bp, Artificial Sequence
  Description of Artificial Sequence Synthetic polynucleotide
acttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccataccgggaaaccaccgcgcccagcttaattttatgagtaacgaagatttattcatttgcatcgaccacgtcgcgtatgcgtgcccggatgccgatgaagcttctaagtattaccaggaaacattcggttggcacgagttgcaccgcgaagagaatccagaacagggcgtggtggaaattatgatggcgcctgctgcgaaattgacggagcacatgactcaggtgcaagttatggcgcctttgaacgatgagagtacggtcgcgaagtggcttgcgaaacacaatgggcgtgctggattgcaccacatggcatggcgtgttgatgacatcgacgcagtgtccgcaacacttcgcgagcgcggtgtacagttgctttacgacgagccgaaactgggtacaggtgggaatcgtatcaacttcatgcatccgaaatctggtaaaggcgtgctgattgaactgacccagtaccccaagaattgataaaggtttttcctaagacgctagcgcataaggtccaccaaatgtcaagtacagaccaaggcacgaaccctgctgacacggatgatttaacgccaaccacattatccctggctggtgatttccctaaggctacggaagagcagtgggagcgcgaggttgaaaaggtgttgaaccgtgggcgcccacccgagaagcagttgacgtttgctgaatgtttaaaacgtcttactgtgcacacagtagatggcattgacatcgttccaatgtatcgcccgaaggatgcccctaagaaactggggtatccaggggttgctccctttacgcgtggcactacggttcgcaatggggatatggacgcttgggacgttcgcgccctgcacgaagaccctgatgaaaaattcacgcgcaaagctattctggaggggctggagcgcggcgtaacaagtttgcttcttcgtgtggaccctgatgcaatcgctcccgaacacttagacgaagtgttaagtgacgttttgctggaaatgaccaaggttgaggtgttttcccgctatgatcagggagctgcggctgaagctcttgtctcggtatatgagcgcagcgacaaaccggctaaagatttggccttaaatttgggactggacccaatcgcatttgctgcacttcagggcactgagccagacttgaccgtacttggtgattgggttcgtcgtttggctaaattcagcccagactcacgcgctgtaacaattgatgctaatatttatcacaacgccggtgcaggcgacgttgccgagctggcctgggcacttgcgaccggagcagagtacgtccgtgcgctggtagagcaaggattcaccgccacagaggcatttgataccattaacttccgtgtgacagcgacccatgatcaatttttaacgattgcccgccttcgtgcgttacgtgaagcgtgggctcgtatcggtgaggtattcggagtagatgaggataaacgtggagcgcgccagaatgctattacgtcctggcgtgaactgacacgcgaggatccctatgtgaacattttacgtggaagtattgccacgttctctgcgtccgttgggggcgcggagtctattaccactttgccattcacgcaggcattgggccttccagaggatgattttccattacgtatcgcacgtaatacaggaattgtcttagctgaggaggtaaacattgggcgtgtaaatgaccctgccggggggtcatactatgtggagagcttgactcgttctcttgcagatgcagcatggaaagagttccaagaggttgaaaagttgggtggtatgtctaaggccgtcatgaccgaacacgtcacgaaggttttagatgcttgcaacgcagagcgcgcgaagcgcttggccaaccgcaagcaacctattacggcagtttccgaatttccgatgattggcgcacgcagcattgagacgaaaccatttccggctgctccggcccgtaaagggctggcatggcaccgcgattccgaagtcttcgagcaacttatggaccgctccacgtcagtttcagagcgtccgaaagtatttttagcatgtcttgggacgcgccgcgattttggaggacgcgaaggattttcatctccggtttggcacattgccgggattgacacgcctcaagtagaaggtgggacgactgctgaaatcgtggaagcgttcaaaaaatctggggcccaagtcgccgatttatgttcgagtgccaaagtgtatgctcaacaaggcttagaggtggcaaaggctctgaaagcggctggggctaaggcgctgtatttgagcggagcatttaaggagttcggagacgatgcagcggaagccgaaaaacttatcgacggacgccttttcatgggcatggatgtcgttgacaccctgtcttccactttagatatccttggagtggcgaagtgataagcttaaaacaatttacatccggccggaacttactatgtctaccttacctcgctttgacagtgttgatttaggaaatgcgccggtcccagcagatgctgcacgtcgttttgaggaacttgcggcgaaagccgggaccggcgaagcctgggaaactgcggaacaaattccagtaggcacgttgtttaatgaagacgtatacaaggacatggattggcttgatacttacgctggcattcctcccttcgtccatggtccgtacgctactatgtatgcatttcgtccttggaccattcgccaatatgccggtttttcgactgcaaaggagtcaaacgcattttaccgtcgtaatttggctgcaggccagaaaggtcttagtgttgcttttgacttacccactcaccgcggttatgattccgacaacccccgcgtggccggagatgttggtatggccggtgtggctatcgattcgatttatgacatgcgtgagctgttcgccggcatcccattagatcagatgagcgtgtcgatgacaatgaacggtgctgtcttgccgattttggctctttatgtggttacggcggaggagcaaggcgtgaagccagaacaactggcgggtactattcaaaatgatattctgaaggaatttatggttcgtaatacatatatttacccgccgcaacctagtatgcgcattatcagcgagatttttgcatacacatcagcaaacatgccgaagtggaactccattagtatcagcggctatcatatgcaggaggctggagcgactgcggatatcgagatggcgtataccttagctgatggagttgattacatccgtgctggtgagtcagtaggacttaatgtggaccaatttgctccacgcctgtccttcttctggggcattggtatgaactttttcatggaggtagcgaagttacgcgctgcccgtatgctgtgggcgaagcttgtccaccagttcggcccgaaaaacccgaagagtatgtctctgcgcacgcactctcaaacatcgggttggtctttgacagctcaagacgtatataataacgttgtacgtacatgcatcgaagccatggctgctactcaaggccatactcaatcacttcatacaaattcgttggatgaagccattgcattgcctacggacttttcagcccgcattgcccgcaatactcaattatttctgcaacaagagagcgggacgactcgtgtgatcgacccttggtcaggttccgcatacgtcgaagagttgacttgggatttagctcgtaaagcctgggggcatattcaggaggttgagaaggtggggggcatggctaaggcaatcgagaaggggattccgaagatgcgcattgaggaggcagccgcccgtacccaagcacgtattgattcgggacgccagccattaattggggtcaataaataccgtctggagcacgaaccacccctggatgtgttgaaggtagacaatagcaccgtgttagctgagcaaaaggccaaacttgttaaattgcgcgcagaacgcgacccagaaaaggtcaaggctgctctggacaaaatcacttgggcggctggcaatcctgatgataaagaccctgatcgcaacttattaaagctgtgcattgatgcggggcgcgcgatggcaacggtaggagagatgagtgacgctttagagaaagtttttgggcgctacacagcgcaaattcgcactatttcaggagtatattcaaaagaagtcaaaaacactccggaagtcgaggaggctcgcgaactggtagaagagtttgagcaggccgaaggccgtcgcccacgtatcctgctggctaaaatggggcaggacggtcatgaccgtgggcaaaaggtcatcgcgactgcatacgccgatttgggatttgacgtggacgttggcccgttattccaaactcccgaggaaactgctcgccaagccgtcgaagccgatgtgcacgtagtgggggtgagctctctggcgggagggcatcttacgcttgtgcctgcgcttcgcaaagagctggacaagttgggtcgtccagatattctgattaccgtaggaggggttattcccgagcaggacttcgatgagcttcgtaaggatggcgctgttgaaatctacacaccggggacggtcattccagaatcggctatctctttagttaaaaaattgcgcgcctccctggatgct

SEQ ID NO: 31 - DNA, 4654 bp, Artificial Sequence
  Description of Artificial Sequence Synthetic polynucleotide
gggaaaccaccgcgcccagcttaattttatgagtaacgaagatttattcatttgcatcgaccacgtcgcgtatgcgtgcccggatgccgatgaagcttctaagtattaccaggaaacattcggttggcacgagttgcaccgcgaagagaatccagaacagggcgtggtggaaattatgatggcgcctgctgcgaaattgacggagcacatgactcaggtgcaagttatggcgcctttgaacgatgagagtacggtcgcgaagtggcttgcgaaacacaatgggcgtgctggattgcaccacatggcatggcgtgttgatgacatcgacgcagtgtccgcaacacttcgcgagcgcggtgtacagttgctttacgacgagccgaaactgggtacaggtgggaatcgtatcaacttcatgcatccgaaatctggtaaaggcgtgctgattgaactgacccagtaccccaagaattgataaaggtttttcctaagacgctagcgcataaggtccaccaaatgtcaagtacagaccaaggcacgaaccctgctgacacggatgatttaacgccaaccacattatccctggctggtgatttccctaaggctacggaagagcagtgggagcgcgaggttgaaaaggtgttgaaccgtgggcgcccacccgagaagcagttgacgtttgctgaatgtttaaaacgtcttactgtgcacacagtagatggcattgacatcgttccaatgtatcgcccgaaggatgcccctaagaaactggggtatccaggggttgctccctttacgcgtggcactacggttcgcaatggggatatggacgcttgggacgttcgcgccctgcacgaagaccctgatgaaaaattcacgcgcaaagctattctggaggggctggagcgcggcgtaacaagtttgcttcttcgtgtggaccctgatgcaatcgctcccgaacacttagacgaagtgttaagtgacgttttgctggaaatgaccaaggttgaggtgttttcccgctatgatcagggagctgcggctgaagctcttgtctcggtatatgagcgcagcgacaaaccggctaaagatttggccttaaatttgggactggacccaatcgcatttgctgcacttcagggcactgagccagacttgaccgtacttggtgattgggttcgtcgtttggctaaattcagcccagactcacgcgctgtaacaattgatgctaatatttatcacaacgccggtgcaggcgacgttgccgagctggcctgggcacttgcgaccggagcagagtacgtccgtgcgctggtagagcaaggattcaccgccacagaggcatttgataccattaacttccgtgtgacagcgacccatgatcaatttttaacgattgcccgccttcgtgcgttacgtgaagcgtgggctcgtatcggtgaggtattcggagtagatgaggataaacgtggagcgcgccagaatgctattacgtcctggcgtgaactgacacgcgaggatccctatgtgaacattttacgtggaagtattgccacgttctctgcgtccgttgggggcgcggagtctattaccactttgccattcacgcaggcattgggccttccagaggatgattttccattacgtatcgcacgtaatacaggaattgtcttagctgaggaggtaaacattgggcgtgtaaatgaccctgccggggggtcatactatgtggagagcttgactcgttctcttgcagatgcagcatggaaagagttccaagaggttgaaaagttgggtggtatgtctaaggccgtcatgaccgaacacgtcacgaaggttttagatgcttgcaacgcagagcgcgcgaagcgcttggccaaccgcaagcaacctattacggcagtttccgaatttccgatgattggcgcacgcagcattgagacgaaaccatttccggctgctccggcccgtaaagggctggcatggcaccgcgattccgaagtcttcgagcaacttatggaccgctccacgtcagtttcagagcgtccgaaagtatttttagcatgtcttgggacgcgccgcgattttggaggacgcgaaggattttcatctccggtttggcacattgccgggattgacacgcctcaagtagaaggtgggacgactgctgaaatcgtggaagcgttcaaaaaatctggggcccaagtcgccgatttatgttcgagtgccaaagtgtatgctcaacaaggcttagaggtggcaaaggctctgaaagcggctggggctaaggcgctgtatttgagcggagcatttaaggagttcggagacgatgcagcggaagccgaaaaacttatcgacggacgccttttcatgggcatggatgtcgttgacaccctgtcttccactttagatatccttggagtggcgaagtgataagcttaaaacaatttacatccggccggaacttactatgtctaccttacctcgctttgacagtgttgatttaggaaatgcgccggtcccagcagatgctgcacgtcgttttgaggaacttgcggcgaaagccgggaccggcgaagcctgggaaactgcggaacaaattccagtaggcacgttgtttaatgaagacgtatacaaggacatggattggcttgatacttacgctggcattcctcccttcgtccatggtccgtacgctactatgtatgcatttcgtccttggaccattcgccaatatgccggtttttcgactgcaaaggagtcaaacgcattttaccgtcgtaatttggctgcaggccagaaaggtcttagtgttgcttttgacttacccactcaccgcggttatgattccgacaacccccgcgtggccggagatgttggtatggccggtgtggctatcgattcgatttatgacatgcgtgagctgttcgccggcatcccattagatcagatgagcgtgtcgatgacaatgaacggtgctgtcttgccgattttggctctttatgtggttacggcggaggagcaaggcgtgaagccagaacaactggcgggtactattcaaaatgatattctgaaggaatttatggttcgtaatacatatatttacccgccgcaacctagtatgcgcattatcagcgagatttttgcatacacatcagcaaacatgccgaagtggaactccattagtatcagcggctatcatatgcaggaggctggagcgactgcggatatcgagatggcgtataccttagctgatggagttgattacatccgtgctggtgagtcagtaggacttaatgtggaccaatttgctccacgcctgtccttcttctggggcattggtatgaactttttcatggaggtagcgaagttacgcgctgcccgtatgctgtgggcgaagcttgtccaccagttcggcccgaaaaacccgaagagtatgtctctgcgcacgcactctcaaacatcgggttggtctttgacagctcaagacgtatataataacgttgtacgtacatgcatcgaagccatggctgctactcaaggccatactcaatcacttcatacaaattcgttggatgaagccattgcattgcctacggacttttcagcccgcattgcccgcaatactcaattatttctgcaacaagagagcgggacgactcgtgtgatcgacccttggtcaggttccgcatacgtcgaagagttgacttgggatttagctcgtaaagcctgggggcatattcaggaggttgagaaggtggggggcatggctaaggcaatcgagaaggggattccgaagatgcgcattgaggaggcagccgcccgtacccaagcacgtattgattcgggacgccagccattaattggggtcaataaataccgtctggagcacgaaccacccctggatgtgttgaaggtagacaatagcaccgtgttagctgagcaaaaggccaaacttgttaaattgcgcgcagaacgcgacccagaaaaggtcaaggctgctctggacaaaatcacttgggcggctggcaatcctgatgataaagaccctgatcgcaacttattaaagctgtgcattgatgcggggcgcgcgatggcaacggtaggagagatgagtgacgctttagagaaagtttttgggcgctacacagcgcaaattcgcactatttcaggagtatattcaaaagaagtcaaaaacactccggaagtcgaggaggctcgcgaactggtagaagagtttgagcaggccgaaggccgtcgcccacgtatcctgctggctaaaatggggcaggacggtcatgaccgtgggcaaaaggtcatcgcgactgcatacgccgatttgggatttgacgtggacgttggcccgttattccaaactcccgaggaaactgctcgccaagccgtcgaagccgatgtgcacgtagtgggggtgagctctctggcgggagggcatcttacgcttgtgcctgcgcttcgcaaagagctggacaagttgggtcgtccagatattctgattaccgtaggaggggttattcccgagcaggacttcgatgagcttcgtaaggatggcgctgttgaaatctacacaccggggacggtcattccagaatcggctatctctttagttaaaaaattgcgcgcctccctggatgct

SEQ ID NO: 32 - DNA, 447 bp, Artificial Sequence
  Description of Artificial Sequence Synthetic polynucleotide
atgagtaacgaagatttattcatttgcatcgaccacgtcgcgtatgcgtgcccggatgccgatgaagcttctaagtattaccaggaaacattcggttggcacgagttgcaccgcgaagagaatccagaacagggcgtggtggaaattatgatggcgcctgctgcgaaattgacggagcacatgactcaggtgcaagttatggcgcctttgaacgatgagagtacggtcgcgaagtggcttgcgaaacacaatgggcgtgctggattgcaccacatggcatggcgtgttgatgacatcgacgcagtgtccgcaacacttcgcgagcgcggtgtacagttgctttacgacgagccgaaactgggtacaggtgggaatcgtatcaacttcatgcatccgaaatctggtaaaggcgtgctgattgaactgacccagtaccccaagaattga

SEQ ID NO: 33 - DNA, 1917 bp, Artificial Sequence
  Description of Artificial Sequence Synthetic polynucleotide
atgtcaagtacagaccaaggcacgaaccctgctgacacggatgatttaacgccaaccacattatccctggctggtgatttccctaaggctacggaagagcagtgggagcgcgaggttgaaaaggtgttgaaccgtgggcgcccacccgagaagcagttgacgtttgctgaatgtttaaaacgtcttactgtgcacacagtagatggcattgacatcgttccaatgtatcgcccgaaggatgcccctaagaaactggggtatccaggggttgctccctttacgcgtggcactacggttcgcaatggggatatggacgcttgggacgttcgcgccctgcacgaagaccctgatgaaaaattcacgcgcaaagctattctggaggggctggagcgcggcgtaacaagtttgcttcttcgtgtggaccctgatgcaatcgctcccgaacacttagacgaagtgttaagtgacgttttgctggaaatgaccaaggttgaggtgttttcccgctatgatcagggagctgcggctgaagctcttgtctcggtatatgagcgcagcgacaaaccggctaaagatttggccttaaatttgggactggacccaatcgcatttgctgcacttcagggcactgagccagacttgaccgtacttggtgattgggttcgtcgtttggctaaattcagcccagactcacgcgctgtaacaattgatgctaatatttatcacaacgccggtgcaggcgacgttgccgagctggcctgggcacttgcgaccggagcagagtacgtccgtgcgctggtagagcaaggattcaccgccacagaggcatttgataccattaacttccgtgtgacagcgacccatgatcaatttttaacgattgcccgccttcgtgcgttacgtgaagcgtgggctcgtatcggtgaggtattcggagtagatgaggataaacgtggagcgcgccagaatgctattacgtcctggcgtgaactgacacgcgaggatccctatgtgaacattttacgtggaagtattgccacgttctctgcgtccgttgggggcgcggagtctattaccactttgccattcacgcaggcattgggccttccagaggatgattttccattacgtatcgcacgtaatacaggaattgtcttagctgaggaggtaaacattgggcgtgtaaatgaccctgccggggggtcatactatgtggagagcttgactcgttctcttgcagatgcagcatggaaagagttccaagaggttgaaaagttgggtggtatgtctaaggccgtcatgaccgaacacgtcacgaaggttttagatgcttgcaacgcagagcgcgcgaagcgcttggccaaccgcaagcaacctattacggcagtttccgaatttccgatgattggcgcacgcagcattgagacgaaaccatttccggctgctccggcccgtaaagggctggcatggcaccgcgattccgaagtcttcgagcaacttatggaccgctccacgtcagtttcagagcgtccgaaagtatttttagcatgtcttgggacgcgccgcgattttggaggacgcgaaggattttcatctccggtttggcacattgccgggattgacacgcctcaagtagaaggtgggacgactgctgaaatcgtggaagcgttcaaaaaatctggggcccaagtcgccgatttatgttcgagtgccaaagtgtatgctcaacaaggcttagaggtggcaaaggctctgaaagcggctggggctaaggcgctgtatttgagcggagcatttaaggagttcggagacgatgcagcggaagccgaaaaacttatcgacggacgccttttcatgggcatggatgtcgttgacaccctgtcttccactttagatatccttggagtggcgaagtga

SEQ ID NO: 34 - DNA, 2184 bp, Artificial Sequence
  Description of Artificial Sequence Synthetic polynucleotide
atgtctaccttacctcgctttgacagtgttgatttaggaaatgcgccggtcccagcagatgctgcacgtcgttttgaggaacttgcggcgaaagccgggaccggcgaagcctgggaaactgcggaacaaattccagtaggcacgttgtttaatgaagacgtatacaaggacatggattggcttgatacttacgctggcattcctcccttcgtccatggtccgtacgctactatgtatgcatttcgtccttggaccattcgccaatatgccggtttttcgactgcaaaggagtcaaacgcattttaccgtcgtaatttggctgcaggccagaaaggtcttagtgttgcttttgacttacccactcaccgcggttatgattccgacaacccccgcgtggccggagatgttggtatggccggtgtggctatcgattcgatttatgacatgcgtgagctgttcgccggcatcccattagatcagatgagcgtgtcgatgacaatgaacggtgctgtcttgccgattttggctctttatgtggttacggcggaggagcaaggcgtgaagccagaacaactggcgggtactattcaaaatgatattctgaaggaatttatggttcgtaatacatatatttacccgccgcaacctagtatgcgcattatcagcgagatttttgcatacacatcagcaaacatgccgaagtggaactccattagtatcagcggctatcatatgcaggaggctggagcgactgcggatatcgagatggcgtataccttagctgatggagttgattacatccgtgctggtgagtcagtaggacttaatgtggaccaatttgctccacgcctgtccttcttctggggcattggtatgaactttttcatggaggtagcgaagttacgcgctgcccgtatgctgtgggcgaagcttgtccaccagttcggcccgaaaaacccgaagagtatgtctctgcgcacgcactctcaaacatcgggttggtctttgacagctcaagacgtatataataacgttgtacgtacatgcatcgaagccatggctgctactcaaggccatactcaatcacttcatacaaattcgttggatgaagccattgcattgcctacggacttttcagcccgcattgcccgcaatactcaattatttctgcaacaagagagcgggacgactcgtgtgatcgacccttggtcaggttccgcatacgtcgaagagttgacttgggatttagctcgtaaagcctgggggcatattcaggaggttgagaaggtggggggcatggctaaggcaatcgagaaggggattccgaagatgcgcattgaggaggcagccgcccgtacccaagcacgtattgattcgggacgccagccattaattggggtcaataaataccgtctggagcacgaaccacccctggatgtgttgaaggtagacaatagcaccgtgttagctgagcaaaaggccaaacttgttaaattgcgcgcagaacgcgacccagaaaaggtcaaggctgctctggacaaaatcacttgggcggctggcaatcctgatgataaagaccctgatcgcaacttattaaagctgtgcattgatgcggggcgcgcgatggcaacggtaggagagatgagtgacgctttagagaaagtttttgggcgctacacagcgcaaattcgcactatttcaggagtatattcaaaagaagtcaaaaacactccggaagtcgaggaggctcgcgaactggtagaagagtttgagcaggccgaaggccgtcgcccacgtatcctgctggctaaaatggggcaggacggtcatgaccgtgggcaaaaggtcatcgcgactgcatacgccgatttgggatttgacgtggacgttggcccgttattccaaactcccgaggaaactgctcgccaagccgtcgaagccgatgtgcacgtagtgggggtgagctctctggcgggagggcatcttacgcttgtgcctgcgcttcgcaaagagctggacaagttgggtcgtccagatattctgattaccgtaggaggggttattcccgagcaggacttcgatgagcttcgtaaggatggcgctgttgaaatctacacaccggggacggtcattccagaatcggctatctctttagttaaaaaattgcgcgcctccctggatgct

SEQ ID NO: 35 - DNA, 6242 bp, Artificial Sequence
  Description of Artificial Sequence Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaataaggcgtaagttcaacaggagagcatttaaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtagtactagattcaatatagagtaaaagaggtaagagtatccatgcgtaaagttctgatcgctaatcgtggagaaattgctgtacgtgtagcacgtgcatgtcgtgatgcgggaatcgcatcagtagccgtatacgcggacccggatcgtgacgcgttgcatgtgcgcgcggcggacgaagcatttgcactgggtggtgatacgcctgcaacatcttacttagacatcgccaaggtgttaaaggctgcacgtgagagtggtgcagacgccattcatcccggttacggctttttaagtgaaaatgccgagttcgcgcaggccgtgttagatgcgggtcttatctggatcggaccaccgccccatgcaatccgcgatcgtggggaaaaagttgcagctcgccatattgcccagcgtgctggggcgccgctggttgcgggcacccctgacccggtttctggtgctgacgaagtcgtcgccttcgcgaaagagcatggactgccgatcgcgattaaggctgcttttggaggcggtggtcgtggtttaaaggttgcccgtacattggaagaagtgcccgagttatatgactccgccgtgcgtgaagctgtggcggcattcggacgtggcgaatgtttcgtggagcgctatttagacaaaccgcgtcatgtagaaacccagtgcttggcagatactcacggtaatgtagttgtggtttctactcgcgactgttcgttacagcgtcgtcatcagaaactggtagaggaggcacccgccccgtttttaagcgaagctcagacagagcaactgtactcctcctccaaggctattcttaaggaagctgggtatggtggagcgggaaccgttgagtttttagtaggtatggatggtactatcttcttcttggaggtcaatacccgcctgcaggtggagcaccctgtgaccgaagaagtcgcagggatcgacctggtccgtgaaatgttccgcattgcagatggcgaggagctggggtacgacgatccagcccttcgcggccactcgttcgaatttcgcatcaatggggaggacccaggtcgtggttttttgcccgcacctggtacggttacgctttttgatgctccgaccggacccggagtccgcctggatgccggggttgagtcaggttccgtaatcggaccggcatgggactcactgctggctaaacttatcgttaccgggcgtacacgtgccgaggcgcttcagcgcgcagcccgcgccttagatgaatttacggttgagggcatggcaaccgcgatccctttccatcgcacagtagtacgcgatccagcattcgctcctgagcttaccgggtcaacggacccattcaccgttcatacacgctggattgaaactgaatttgtcaacgaaattaagccttttaccacccctgccgacacggagacagatgaagagtctgggcgcgagacagtggtagtcgaggtcggtgggaaacgcttagaggtaagtcttccgtccagcctgggaatgtcgttggcccgtaccggccttgccgcgggggcccgccccaaacgccgcgcggccaagaagtcaggccctgcagcatcgggtgatacactggcatctcctatgcaaggtacgatcgtaaagatcgccgtggaagagggacaagaagtacaggagggagatctgattgtggttcttgaagctatgaagatggaacagccacttaatgcccaccgttcgggaaccattaaggggcttactgctgaagtaggtgcttcactgacgtcgggcgccgctatctgtgaaatcaaggattgataacgctaacgaaaaagttaaatacaggaacaagagaacatatgtcggagcccgaggaacagcagccagatatccacacgacagcgggcaagttagctgatcttcgtcgccgcatcgaagaggcaacgcacgccggttctgcgcgcgcggtggagaaacagcacgcgaagggtaaacttacggctcgtgagcgtatcgatttgttgctggacgaagggtcttttgtagagcttgatgagtttgcgcgtcaccgttcgacgaatttcggactggatgccaaccgtccatatggagatggagtggtgactggctatggaactgttgacggacgtccggttgccgtcttttcgcaagactttacggtctttgggggcgctctgggggaagtatacgggcaaaaaattgtgaaggtcatggatttcgctcttaagaccgggtgtcccgtcgtgggtattaatgactcaggtggggcacgcattcaagagggtgtagcaagtctgggcgcgtatggagagattttccgtcgcaatacgcacgcgtcgggcgtgatccctcagatttcgcttgtagttggcccatgcgcagggggagctgtgtactctccagctattactgactttacggtaatggtcgaccaaacatcgcatatgtttatcaccggacccgatgtgattaagacagtgacaggggaggatgtgggttttgaggaacttggtggtgcgcgtacgcacaacagtacgtctggggttgcccatcatatggctggggatgagaaagacgctgtggagtatgttaagcaattattgagttatttgccgtcgaacaatttaagtgagcctccggcgtttcctgaagaggctgatttagccgttacggacgaagatgcggaattagatacaattgtgccggattcggctaaccaaccctatgatatgcattctgtaatcgagcatgtccttgacgatgcggaatttttcgagactcaaccgttgtttgcccccaacatcctgaccggctttggtcgcgttgaaggccgtccggtgggtatcgtggcgaatcagccgatgcagtttgctggatgcttagatatcactgcctcagaaaaagctgctcgtttcgttcgcacttgcgacgctttcaacgtccctgtgcttacgtttgtagacgtccccgggtttttaccgggcgtagatcaggagcatgacgggatcatccgccgcggtgcgaagttgatttttgcctatgcagaagcgaccgtgccgttgatcacagtaatcacgcgcaaagccttcggaggtgcgtatgacgtaatgggctcaaaacaccttggcgctgaccttaatctggcatggcccacggcccaaatcgctgtaatgggcgctcaaggtgctgtaaacatccttcatcgtcgtacgattgcagatgcgggggacgatgcggaagccacgcgcgcccgtttaattcaagagtacgaggatgctttattaaatccctatactgcggctgagcgcgggtatgtagacgcggtcatcatgccctcagatactcgccgtcatatcgtacgtggtttacgccaattacgcaccaagcgcgagtctttacccccgaaaaagcacgggaacattcccctttgaggaggtcggataaggcgctcgcgccgcatccgacaccgtgcgcagatgcctgatgcgacgctgacgcgtcttatcatgcctcgctctcgagtcccgtcaagtcagcgtaatgctctgccagtgttacaaccaattaaccaattctgat

SEQ ID NO: 36 - DNA, 5464 bp, Artificial Sequence
  Description of Artificial Sequence Synthetic polynucleotide
taattcctaatttttgttgacactctatcattgatagagttattttaccactccctatcagtgatagagaaaagtgaataaggcgtaagttcaacaggagagcatttaaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtagtactagattcaatatagagtaaaagaggtaagagtatccatgcgtaaagttctgatcgctaatcgtggagaaattgctgtacgtgtagcacgtgcatgtcgtgatgcgggaatcgcatcagtagccgtatacgcggacccggatcgtgacgcgttgcatgtgcgcgcggcggacgaagcatttgcactgggtggtgatacgcctgcaacatcttacttagacatcgccaaggtgttaaaggctgcacgtgagagtggtgcagacgccattcatcccggttacggctttttaagtgaaaatgccgagttcgcgcaggccgtgttagatgcgggtcttatctggatcggaccaccgccccatgcaatccgcgatcgtggggaaaaagttgcagctcgccatattgcccagcgtgctggggcgccgctggttgcgggcacccctgacccggtttctggtgctgacgaagtcgtcgccttcgcgaaagagcatggactgccgatcgcgattaaggctgcttttggaggcggtggtcgtggtttaaaggttgcccgtacattggaagaagtgcccgagttatatgactccgccgtgcgtgaagctgtggcggcattcggacgtggcgaatgtttcgtggagcgctatttagacaaaccgcgtcatgtagaaacccagtgcttggcagatactcacggtaatgtagttgtggtttctactcgcgactgttcgttacagcgtcgtcatcagaaactggtagaggaggcacccgccccgtttttaagcgaagctcagacagagcaactgtactcctcctccaaggctattcttaaggaagctgggtatggtggagcgggaaccgttgagtttttagtaggtatggatggtactatcttcttcttggaggtcaatacccgcctgcaggtggagcaccctgtgaccgaagaagtcgcagggatcgacctggtccgtgaaatgttccgcattgcagatggcgaggagctggggtacgacgatccagcccttcgcggccactcgttcgaatttcgcatcaatggggaggacccaggtcgtggttttttgcccgcacctggtacggttacgctttttgatgctccgaccggacccggagtccgcctggatgccggggttgagtcaggttccgtaatcggaccggcatgggactcactgctggctaaacttatcgttaccgggcgtacacgtgccgaggcgcttcagcgcgcagcccgcgccttagatgaatttacggttgagggcatggcaaccgcgatccctttccatcgcacagtagtacgcgatccagcattcgctcctgagcttaccgggtcaacggacccattcaccgttcatacacgctggattgaaactgaatttgtcaacgaaattaagccttttaccacccctgccgacacggagacagatgaagagtctgggcgcgagacagtggtagtcgaggtcggtgggaaacgcttagaggtaagtcttccgtccagcctgggaatgtcgttggcccgtaccggccttgccgcgggggcccgccccaaacgccgcgcggccaagaagtcaggccctgcagcatcgggtgatacactggcatctcctatgcaaggtacgatcgtaaagatcgccgtggaagagggacaagaagtacaggagggagatctgattgtggttcttgaagctatgaagatggaacagccacttaatgcccaccgttcgggaaccattaaggggcttactgctgaagtaggtgcttcactgacgtcgggcgccgctatctgtgaaatcaaggattgataacgctaacgaaaaagttaaatacaggaacaagagaacatatgtcggagcccgaggaacagcagccagatatccacacgacagcgggcaagttagctgatcttcgtcgccgcatcgaagaggcaacgcacgccggttctgcgcgcgcggtggagaaacagcacgcgaagggtaaacttacggctcgtgagcgtatcgatttgttgctggacgaagggtcttttgtagagcttgatgagtttgcgcgtcaccgttcgacgaatttcggactggatgccaaccgtccatatggagatggagtggtgactggctatggaactgttgacggacgtccggttgccgtcttttcgcaagactttacggtctttgggggcgctctgggggaagtatacgggcaaaaaattgtgaaggtcatggatttcgctcttaagaccgggtgtcccgtcgtgggtattaatgactcaggtggggcacgcattcaagagggtgtagcaagtctgggcgcgtatggagagattttccgtcgcaatacgcacgcgtcgggcgtgatccctcagatttcgcttgtagttggcccatgcgcagggggagctgtgtactctccagctattactgactttacggtaatggtcgaccaaacatcgcatatgtttatcaccggacccgatgtgattaagacagtgacaggggaggatgtgggttttgaggaacttggtggtgcgcgtacgcacaacagtacgtctggggttgcccatcatatggctggggatgagaaagacgctgtggagtatgttaagcaattattgagttatttgccgtcgaacaatttaagtgagcctccggcgtttcctgaagaggctgatttagccgttacggacgaagatgcggaattagatacaattgtgccggattcggctaaccaaccctatgatatgcattctgtaatcgagcatgtccttgacgatgcggaatttttcgagactcaaccgttgtttgcccccaacatcctgaccggctttggtcgcgttgaaggccgtccggtgggtatcgtggcgaatcagccgatgcagtttgctggatgcttagatatcactgcctcagaaaaagctgctcgtttcgttcgcacttgcgacgctttcaacgtccctgtgcttacgtttgtagacgtccccgggtttttaccgggcgtagatcaggagcatgacgggatcatccgccgcggtgcgaagttgatttttgcctatgcagaagcgaccgtgccgttgatcacagtaatcacgcgcaaagccttcggaggtgcgtatgacgtaatgggctcaaaacaccttggcgctgaccttaatctggcatggcccacggcccaaatcgctgtaatgggcgctcaaggtgctgtaaacatccttcatcgtcgtacgattgcagatgcgggggacgatgcggaagccacgcgcgcccgtttaattcaagagtacgaggatgctttattaaatccctatactgcggctgagcgcgggtatgtagacgcggtcatcatgccctcagatactcgccgtcatatcgtacgtggtttacgccaattacgcaccaagcgcgagtctttacccccgaaaaagcacgggaacattcccctt

SEQ ID NO: 37 - DNA, 5358 bp, Artificial Sequence
  Description of Artificial Sequence Synthetic polynucleotide
taaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtagtactagattcaatatagagtaaaagaggtaagagtatccatgcgtaaagttctgatcgctaatcgtggagaaattgctgtacgtgtagcacgtgcatgtcgtgatgcgggaatcgcatcagtagccgtatacgcggacccggatcgtgacgcgttgcatgtgcgcgcggcggacgaagcatttgcactgggtggtgatacgcctgcaacatcttacttagacatcgccaaggtgttaaaggctgcacgtgagagtggtgcagacgccattcatcccggttacggctttttaagtgaaaatgccgagttcgcgcaggccgtgttagatgcgggtcttatctggatcggaccaccgccccatgcaatccgcgatcgtggggaaaaagttgcagctcgccatattgcccagcgtgctggggcgccgctggttgcgggcacccctgacccggtttctggtgctgacgaagtcgtcgccttcgcgaaagagcatggactgccgatcgcgattaaggctgcttttggaggcggtggtcgtggtttaaaggttgcccgtacattggaagaagtgcccgagttatatgactccgccgtgcgtgaagctgtggcggcattcggacgtggcgaatgtttcgtggagcgctatttagacaaaccgcgtcatgtagaaacccagtgcttggcagatactcacggtaatgtagttgtggtttctactcgcgactgttcgttacagcgtcgtcatcagaaactggtagaggaggcacccgccccgtttttaagcgaagctcagacagagcaactgtactcctcctccaaggctattcttaaggaagctgggtatggtggagcgggaaccgttgagtttttagtaggtatggatggtactatcttcttcttggaggtcaatacccgcctgcaggtggagcaccctgtgaccgaagaagtcgcagggatcgacctggtccgtgaaatgttccgcattgcagatggcgaggagctggggtacgacgatccagcccttcgcggccactcgttcgaatttcgcatcaatggggaggacccaggtcgtggttttttgcccgcacctggtacggttacgctttttgatgctccgaccggacccggagtccgcctggatgccggggttgagtcaggttccgtaatcggaccggcatgggactcactgctggctaaacttatcgttaccgggcgtacacgtgccgaggcgcttcagcgcgcagcccgcgccttagatgaatttacggttgagggcatggcaaccgcgatccctttccatcgcacagtagtacgcgatccagcattcgctcctgagcttaccgggtcaacggacccattcaccgttcatacacgctggattgaaactgaatttgtcaacgaaattaagccttttaccacccctgccgacacggagacagatgaagagtctgggcgcgagacagtggtagtcgaggtcggtgggaaacgcttagaggtaagtcttccgtccagcctgggaatgtcgttggcccgtaccggccttgccgcgggggcccgccccaaacgccgcgcggccaagaagtcaggccctgcagcatcgggtgatacactggcatctcctatgcaaggtacgatcgtaaagatcgccgtggaagagggacaagaagtacaggagggagatctgattgtggttcttgaagctatgaagatggaacagccacttaatgcccaccgttcgggaaccattaaggggcttactgctgaagtaggtgcttcactgacgtcgggcgccgctatctgtgaaatcaaggattgataacgctaacgaaaaagttaaatacaggaacaagagaacatatgtcggagcccgaggaacagcagccagatatccacacgacagcgggcaagttagctgatcttcgtcgccgcatcgaagaggcaacgcacgccggttctgcgcgcgcggtggagaaacagcacgcgaagggtaaacttacggctcgtgagcgtatcgatttgttgctggacgaagggtcttttgtagagcttgatgagtttgcgcgtcaccgttcgacgaatttcggactggatgccaaccgtccatatggagatggagtggtgactggctatggaactgttgacggacgtccggttgccgtcttttcgcaagactttacggtctttgggggcgctctgggggaagtatacgggcaaaaaattgtgaaggtcatggatttcgctcttaagaccgggtgtcccgtcgtgggtattaatgactcaggtggggcacgcattcaagagggtgtagcaagtctgggcgcgtatggagagattttccgtcgcaatacgcacgcgtcgggcgtgatccctcagatttcgcttgtagttggcccatgcgcagggggagctgtgtactctccagctattactgactttacggtaatggtcgaccaaacatcgcatatgtttatcaccggacccgatgtgattaagacagtgacaggggaggatgtgggttttgaggaacttggtggtgcgcgtacgcacaacagtacgtctggggttgcccatcatatggctggggatgagaaagacgctgtggagtatgttaagcaattattgagttatttgccgtcgaacaatttaagtgagcctccggcgtttcctgaagaggctgatttagccgttacggacgaagatgcggaattagatacaattgtgccggattcggctaaccaaccctatgatatgcattctgtaatcgagcatgtccttgacgatgcggaatttttcgagactcaaccgttgtttgcccccaacatcctgaccggctttggtcgcgttgaaggccgtccggtgggtatcgtggcgaatcagccgatgcagtttgctggatgcttagatatcactgcctcagaaaaagctgctcgtttcgttcgcacttgcgacgctttcaacgtccctgtgcttacgtttgtagacgtccccgggtttttaccgggcgtagatcaggagcatgacgggatcatccgccgcggtgcgaagttgatttttgcctatgcagaagcgaccgtgccgttgatcacagtaatcacgcgcaaagccttcggaggtgcgtatgacgtaatgggctcaaaacaccttggcgctgaccttaatctggcatggcccacggcccaaatcgctgtaatgggcgctcaaggtgctgtaaacatccttcatcgtcgtacgattgcagatgcgggggacgatgcggaagccacgcgcgcccgtttaattcaagagtacgaggatgctttattaaatccctatactgcggctgagcgcgggtatgtagacgcggtcatcatgccctcagatactcgccgtcatatcgtacgtggtttacgccaattacgcaccaagcgcgagtctttacccccgaaaaagcacgggaacattcccctt

SEQ ID NO: 38 - DNA, 1772 bp, Artificial Sequence
  Description of Artificial Sequence Synthetic polynucleotide
atgcgtaaagttctgatcgctaatcgtggagaaattgctgtacgtgtagcacgtgcatgtcgtgatgcgggaatcgcatcagtagccgtatacgcggacccggatcgtgacgcgttgcatgtgcgcgcggcggacgaagcatttgcactgggtggtgatacgcctgcaacatcttacttagacatcgccaaggtgttaaaggctgcacgtgagagtggtgcagacgccattcatcccggttacggctttttaagtgaaaatgccgagttcgcgcaggccgtgttagatgcgggtcttatctggatcggaccaccgccccatgcaatccgcgatcgtggggaaaaagttgcagctcgccatattgcccagcgtgctggggcgccgctggttgcgggcacccctgacccggtttctggtgctgacgaagtcgtcgccttcgcgaaagagcatggactgccgatcgcgattaaggctgcttttggaggcggtggtcgtggtttaaaggttgcccgtacattggaagaagtgcccgagttatatgactccgccgtgcgtgaagctgtggcggcattcggacgtggcgaatgtttcgtggagcgctatttagacaaaccgcgtcatgtagaaacccagtgcttggcagatactcacggtaatgtagttgtggtttctactcgcgactgttcgttacagcgtcgtcatcagaaactggtagaggaggcacccgccccgtttttaagcgaagctcagacagagcaactgtactcctcctccaaggctattcttaaggaagctgggtatggtggagcgggaaccgttgagtttttagtaggtatggatggtactatcttcttcttggaggtcaatacccgcctgcaggtggagcaccctgtgaccgaagaagtcgcagggatcgacctggtccgtgaaatgttccgcattgcagatggcgaggagctggggtacgacgatccagcccttcgcggccactcgttcgaatttcgcatcaatggggaggacccaggtcgtggttttttgcccgcacctggtacggttacgctttttgatgctccgaccggacccggagtccgcctggatgccggggttgagtcaggttccgtaatcggaccggcatgggactcactgctggctaaacttatcgttaccgggcgtacacgtgccgaggcgcttcagcgcgcagcccgcgccttagatgaatttacggttgagggcatggcaaccgcgatccctttccatcgcacagtagtacgcgatccagcattcgctcctgagcttaccgggtcaacggacccattcaccgttcatacacgctggattgaaactgaatttgtcaacgaaattaagccttttaccacccctgccgacacggagacagatgaagagtctgggcgcgagacagtggtagtcgaggtcggtgggaaacgcttagaggtaagtcttccgtccagcctgggaatgtcgttggcccgtaccggccttgccgcgggggcccgccccaaacgccgcgcggccaagaagtcaggccctgcagcatcgggtgatacactggcatctcctatgcaaggtacgatcgtaaagatcgccgtggaagagggacaagaagtacaggagggagatctgattgtggttcttgaagctatgaagatggaacagccacttaatgcccaccgttcgggaaccattaaggggcttactgctgaagtaggtgcttcactgacgtcgggcgccgctatctgtgaaatcaaggattg

SEQ ID NO: 39 - DNA, 1592 bp, Artificial Sequence
  Description of Artificial Sequence Synthetic polynucleotide
atgtcggagcccgaggaacagcagccagatatccacacgacagcgggcaagttagctgatcttcgtcgccgcatcgaagaggcaacgcacgccggttctgcgcgcgcggtggagaaacagcacgcgaagggtaaacttacggctcgtgagcgtatcgatttgttgctggacgaagggtcttttgtagagcttgatgagtttgcgcgtcaccgttcgacgaatttcggactggatgccaaccgtccatatggagatggagtggtgactggctatggaactgttgacggacgtccggttgccgtcttttcgcaagactttacggtctttgggggcgctctgggggaagtatacgggcaaaaaattgtgaaggtcatggatttcgctcttaagaccgggtgtcccgtcgtgggtattaatgactcaggtggggcacgcattcaagagggtgtagcaagtctgggcgcgtatggagagattttccgtcgcaatacgcacgcgtcgggcgtgatccctcagatttcgcttgtagttggcccatgcgcagggggagctgtgtactctccagctattactgactttacggtaatggtcgaccaaacatcgcatatgtttatcaccggacccgatgtgattaagacagtgacaggggaggatgtgggttttgaggaacttggtggtgcgcgtacgcacaacagtacgtctggggttgcccatcatatggctggggatgagaaagacgctgtggagtatgttaagcaattattgagttatttgccgtcgaacaatttaagtgagcctccggcgtttcctgaagaggctgatttagccgttacggacgaagatgcggaattagatacaattgtgccggattcggctaaccaaccctatgatatgcattctgtaatcgagcatgtccttgacgatgcggaatttttcgagactcaaccgttgtttgcccccaacatcctgaccggctttggtcgcgttgaaggccgtccggtgggtatcgtggcgaatcagccgatgcagtttgctggatgcttagatatcactgcctcagaaaaagctgctcgtttcgttcgcacttgcgacgctttcaacgtccctgtgcttacgtttgtagacgtccccgggtttttaccgggcgtagatcaggagcatgacgggatcatccgccgcggtgcgaagttgatttttgcctatgcagaagcgaccgtgccgttgatcacagtaatcacgcgcaaagccttcggaggtgcgtatgacgtaatgggctcaaaacaccttggcgctgaccttaatctggcatggcccacggcccaaatcgctgtaatgggcgctcaaggtgctgtaaacatccttcatcgtcgtacgattgcagatgcgggggacgatgcggaagccacgcgcgcccgtttaattcaagagtacgaggatgctttattaaatccctatactgcggctgagcgcgggtatgtagacgcggtcatcatgccctcagatactcgccgtcatatcgtacgtggtttacgccaattacgcaccaagcgcgagtctttacccccgaaaaagcacgggaacattcccctttg

SEQ ID NO: 40 - DNA, 2486 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccataccatattcatagaaagaatactaagagaggtcagaatgaaagatgttgttatcgtagccgctaaacgcactgcgatcggttcctttctggggagtctggcttccctgagcgcccctcagttgggtcagacggctatccgcgcagttttggattctgcaaatgtgaaaccagaacaagtggaccaagtaattatggggaatgtgctgaccaccggcgttgggcaaaatcctgctcgtcaggcagcaatcgccgctgggattcctgtacaagttcccgccagcacgcttaatgtagtgtgtgggtccggattacgtgccgttcacctggcagctcaagccatccaatgcgatgaagccgatatcgtcgttgccggaggtcaagaatcaatgtcccagtctgctcattacatgcagcttcgcaatggccagaaaatgggtaacgcacagttagtcgattcaatggtggccgacggcttgaccgacgcgtataatcaataccagatgggtatcaccgcggagaatatcgtcgaaaaacttggtcttaatcgtgaagaacaagaccagcttgctctgacaagtcaacaacgtgctgcagcagcgcaggctgccggaaaattcaaggatgaaattgcggtcgtttcgattccccagcgcaaaggagagccggtcgtcttcgcggaagacgaatatatcaaggccaatacctcgttggaatccttgacgaaactgcgtccagcattcaaaaaagacggttctgttacagccggcaacgcatctggcattaatgatggggcagccgcggtcctgatgatgtccgccgacaaagcggctgaactgggcttaaagcctttagcacgcattaaaggttacgcgatgtcaggaattgagccggaaatcatgggactgggtcctgtagacgccgttaagaaaacccttaataaggctggttggtccttagaccaggtcgatctgatcgaggccaatgaggcttttgctgcccaagcactgggagtagccaaggagcttgggctggacctggacaaggtaaatgttaacggaggtgcgatcgcgctgggacacccgatcggggcttcgggttgtcgtatcttggtcacgttattacacgaaatgcagcgtcgtgatgcaaagaagggtatcgccacattgtgtgtgggaggtggaatgggggtggcgcttgccgttgagcgcgattaaggagctcggtaccaaattccagaaaagagacgctttcgagcgtcttttttcgttttggtccgcgcaataaaaaagcccccggaaggtgatcttccgggggctttctcatgcgtt

SEQ ID NO: 41 - DNA, 2056 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccataccgctagaactagatctagagtaataaggaggaaggaatgtcagagcagaaagtagctctggttaccggtgcgttaggtggtatcggaagtgagatctgccgccagcttgtgaccgccgggtacaagattatcgccaccgttgttccacgcgaagaagaccgcgaaaaacaatggttgcaaagtgaggggtttcaagactctgatgtgcgtttcgtattaacagatttaaacaatcacgaagctgcgacagcggcaattcaagaagcgattgccgccgaaggacgcgttgatgtattggtcaacaacgcggggatcacgcgcgatgctacatttaagaaaatgtcctatgagcaatggtcccaagtcatcgacacgaatttaaagactctttttaccgtgacccagccagtatttaataaaatgcttgaacagaagtctggccgcatcgtaaacattagctctgtcaatggtttaaaagggcaatttggtcaagccaactactcggcctcgaaagcagggattatcgggtttactaaagcattggcgcaggagggtgctcgctcgaacatttgcgtcaatgtcgttgctcctggttacacagcgacacccatggtcacagcaatgcgcgaggatgtaattaagtcaatcgaagctcaaattcccctgcaacgtctggcagcaccggcggagattgcggcagcggttatgtatttggtgagtgaacacggtgcatacgtgacgggcgaaactttgagtatcaacggcgggctgtacatgcactaaggagctcggtaccaaattccagaaaagagacgctttcgagcgtcttttttcgttttggtccgcgcaataaaaaagcccccggaaggtgatcttccgggggctttctcatgcgtt

SEQ ID NO: 42 - DNA, 3081 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccataccactattatttaatatacgacatcaggaggttccaatgaatccaaattcctttcagtttaaagagaatatcttacagtttttcagcgtgcacgacgatatttggaaaaaactgcaggaattttactatggacaatcgcccatcaatgaagcgttggcgcagttaaataaggaagacatgagtttattcttcgaggcgttatcaaaaaaccctgctcgtatgatggagatgcagtggtcctggtggcaagggcagattcaaatttaccagaacgtgttaatgcgtagtgtagccaaggacgtagccccctttatccagccagagtccggagatcgtcgcttcaactcgccactttggcaagaacatccaaattttgatttactgagtcaatcctacttgttgttttctcagttggttcaaaatatggtggatgtcgttgaaggagtacctgataaggtccgctatcgcatccatttctttacacgtcagatgatcaatgcgttgtctccttctaatttcctgtggacgaaccctgaagtaattcaacagacggtcgctgaacagggtgagaatttagtacgcgggatgcaagtatttcacgatgatgtaatgaattcgggtaaatatttgagcatccgtatggtaaatagcgacagtttctctcttggcaaggacttggcgtatacgccaggagccgtagttttcgagaacgacatctttcagcttcttcaatacgaagccacaaccgagaacgtatatcaaacccctattcttgtcgtacctcccttcatcaacaagtactacgtgctggacctgcgcgaacagaatagcttggttaattggctgcgccaacaaggacatacggtgtttttgatgtcgtggcgtaaccccaacgcagagcagaaggagcttaccttcgctgacttaattacccaaggatcggtagaagcattacgtgttatcgaagaaatcacgggagagaaagaagctaactgtattggatattgcatcggtggtacacttctggctgctacccaggcatattatgtagctaaacgcctgaaaaatcacgtaaagtcagcgacttatatggcgacgattattgattttgagaaccccggctcattgggtgttttcattaatgagccggtcgtaagtggacttgaaaaccttaataatcaacttggttacttcgacgggcgtcaacttgcagtgacattttcgttgttgcgcgaaaacaccttgtattggaattattacatcgataattacttgaagggtaaggaaccgtccgactttgacatcttatactggaactcggatggtacgaatatcccagcaaagattcacaatttcctgttacgtaacctttatcttaacaacgaacttatttctccaaatgccgtcaaagttaatggtgtgggtttaaacctttcgcgcgtgaagactccatcattcttcattgctacgcaggaggaccatatcgcattgtgggatacctgttttcgcggcgcggattacctggggggtgagagcacacttgtgcttggggaaagcggacacgtcgccggcattgtcaacccgccttctcgtaacaagtatggttgttacacgaacgccgccaagtttgaaaataccaagcaatggcttgacggtgcagaatatcatcccgaaagctggtggttacgttggcaggcatgggtcacgccttatactggagagcaggttcctgcgcgtaatttgggaaacgcacagtaccccagtattgaagcggcccctgggcgttatgtgctggtaaacctgttttaaggagctcggtaccaaattccagaaaagagacgctttcgagcgtcttttttcgttttggtccgcgcaataaaaaagcccccggaaggtgatcttccgggggctttctcatgcgtt

SEQ ID NO: 43 - DNA, 3187 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccataccagatttaaagtaaggccagggaataaatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtagggagctcggtaccaaattccagaaaagagacgctttcgagcgtcttttttcgttttggtccgcgcaataaaaaagcccccggaaggtgatcttccgggggctttctcatgcgtt

SEQ ID NO: 44 - DNA, 2886 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacccgtttttttggatggagtgaaacgatgtccttcctggtcgagaatcaattgttagcacttgtcgtgatcatgaccgtcgggcttttacttggacgtatcaaaatctttggtttccgtttgggtgtggccgccgtgttgttcgtcggccttgctttaagcaccattgagcccgacatttcggttccatcccttatttacgtggttggcctttcgctttttgtgtatactatcggtctggaagctggccccggtttttttacatctatgaagacgacgggtttgcgcaataacgcactgacgttaggtgccattatcgcgacaacagcacttgcgtgggcactgattaccgtcttgaatattgatgccgcctcaggagctggtatgcttactggtgccttaactaatacgcccgctatggctgcggtagtggatgcacttccctcattaattgatgacacaggccagctgcatcttattgctgagctgccggtggttgcttattccctggcttatcccttgggggtactgattgtgatcttgagcatcgccatcttttcttcagtgtttaaggttgaccataacaaggaggcagaagaggctggggtagcggtccaagaacttaagggccgccgtatccgcgtaactgtagctgacttgccagcccttgagaacattcctgagttgcttaatttacatgttatcgtctcgcgtgtagagcgcgacggagagcagttcatccccttatatggcgaacatgcacgcatcggcgatgtactgactgtcgtgggggccgacgaggaactgaaccgcgcggaaaaagccatcggagagttaattgacggtgatccttactctaacgttgaactggactatcgtcgtatcttcgtctctaatacggcggttgtcggtacacccctgagcaaattgcaaccgctttttaaagatatgcttattactcgcattcgccgcggtgatacggatctggtagcttcctcggacatgacgcttcaattaggcgaccgcgttcgtgtggttgccccagccgagaaacttcgtgaagcgactcagttgcttggagactcttacaaaaagctgtccgactttaatttattgcctcttgctgcgggcttaatgattggcgtccttgttggaatggttgaattcccactgcctggggggtcatctttaaaacttggcaatgccggtggtccgttggttgtcgcgctgttgcttgggatgatcaatcgtacgggaaagttcgtctggcagatcccgtacggagcaaacttggcgttacgtcagttgggtatcaccctgttcttggcggctattggcacttccgcgggagctgggtttcgctcagctattagcgacccgcaatctctgaccattattggatttggtgcgttgttaaccttgtttattagtattaccgtcttgttcgttgggcataagttgatgaaaatcccgtttggggaaacggcgggtatcttagctggaacgcagacccatccagcagtattatcatatgtgtctgacgcatctcgcaacgagttgccagccatggggtacacctcagtgtatcccttggctatgattgcgaaaatcctggctgcacaaacacttttgtttctgttgatttaatgaggaatcgactccacgtccctagcgtgtgtaggctggagctgcttcgaagttcctatactttctagagaataggaacttc

SEQ ID NO: 45 - DNA, 1643 bp, Artificial Sequence
  Description of Artificial Sequence Synthetic polynucleotide
cccgtttttttggatggagtgaaacgatgtccttcctggtcgagaatcaattgttagcacttgtcgtgatcatgaccgtcgggcttttacttggacgtatcaaaatctttggtttccgtttgggtgtggccgccgtgttgttcgtcggccttgctttaagcaccattgagcccgacatttcggttccatcccttatttacgtggttggcctttcgctttttgtgtatactatcggtctggaagctggccccggtttttttacatctatgaagacgacgggtttgcgcaataacgcactgacgttaggtgccattatcgcgacaacagcacttgcgtgggcactgattaccgtcttgaatattgatgccgcctcaggagctggtatgcttactggtgccttaactaatacgcccgctatggctgcggtagtggatgcacttccctcattaattgatgacacaggccagctgcatcttattgctgagctgccggtggttgcttattccctggcttatcccttgggggtactgattgtgatcttgagcatcgccatcttttcttcagtgtttaaggttgaccataacaaggaggcagaagaggctggggtagcggtccaagaacttaagggccgccgtatccgcgtaactgtagctgacttgccagcccttgagaacattcctgagttgcttaatttacatgttatcgtctcgcgtgtagagcgcgacggagagcagttcatccccttatatggcgaacatgcacgcatcggcgatgtactgactgtcgtgggggccgacgaggaactgaaccgcgcggaaaaagccatcggagagttaattgacggtgatccttactctaacgttgaactggactatcgtcgtatcttcgtctctaatacggcggttgtcggtacacccctgagcaaattgcaaccgctttttaaagatatgcttattactcgcattcgccgcggtgatacggatctggtagcttcctcggacatgacgcttcaattaggcgaccgcgttcgtgtggttgccccagccgagaaacttcgtgaagcgactcagttgcttggagactcttacaaaaagctgtccgactttaatttattgcctcttgctgcgggcttaatgattggcgtccttgttggaatggttgaattcccactgcctggggggtcatctttaaaacttggcaatgccggtggtccgttggttgtcgcgctgttgcttgggatgatcaatcgtacgggaaagttcgtctggcagatcccgtacggagcaaacttggcgttacgtcagttgggtatcaccctgttcttggcggctattggcacttccgcgggagctgggtttcgctcagctattagcgacccgcaatctctgaccattattggatttggtgcgttgttaaccttgtttattagtattaccgtcttgttcgttgggcataagttgatgaaaatcccgtttggggaaacggcgggtatcttagctggaacgcagacccatccagcagtattatcatatgtgtctgacgcatctcgcaacgagttgccagccatggggtacacctcagtgtatcccttggctatgattgcgaaaatcctggctgcacaaacacttttgtttctgttgatt

SEQ ID NO: 46 - DNA, 1617 bp, Artificial Sequence
  Description of Artificial Sequence Synthetic polynucleotide
atgtccttcctggtcgagaatcaattgttagcacttgtcgtgatcatgaccgtcgggcttttacttggacgtatcaaaatctttggtttccgtttgggtgtggccgccgtgttgttcgtcggccttgctttaagcaccattgagcccgacatttcggttccatcccttatttacgtggttggcctttcgctttttgtgtatactatcggtctggaagctggccccggtttttttacatctatgaagacgacgggtttgcgcaataacgcactgacgttaggtgccattatcgcgacaacagcacttgcgtgggcactgattaccgtcttgaatattgatgccgcctcaggagctggtatgcttactggtgccttaactaatacgcccgctatggctgcggtagtggatgcacttccctcattaattgatgacacaggccagctgcatcttattgctgagctgccggtggttgcttattccctggcttatcccttgggggtactgattgtgatcttgagcatcgccatcttttcttcagtgtttaaggttgaccataacaaggaggcagaagaggctggggtagcggtccaagaacttaagggccgccgtatccgcgtaactgtagctgacttgccagcccttgagaacattcctgagttgcttaatttacatgttatcgtctcgcgtgtagagcgcgacggagagcagttcatccccttatatggcgaacatgcacgcatcggcgatgtactgactgtcgtgggggccgacgaggaactgaaccgcgcggaaaaagccatcggagagttaattgacggtgatccttactctaacgttgaactggactatcgtcgtatcttcgtctctaatacggcggttgtcggtacacccctgagcaaattgcaaccgctttttaaagatatgcttattactcgcattcgccgcggtgatacggatctggtagcttcctcggacatgacgcttcaattaggcgaccgcgttcgtgtggttgccccagccgagaaacttcgtgaagcgactcagttgcttggagactcttacaaaaagctgtccgactttaatttattgcctcttgctgcgggcttaatgattggcgtccttgttggaatggttgaattcccactgcctggggggtcatctttaaaacttggcaatgccggtggtccgttggttgtcgcgctgttgcttgggatgatcaatcgtacgggaaagttcgtctggcagatcccgtacggagcaaacttggcgttacgtcagttgggtatcaccctgttcttggcggctattggcacttccgcgggagctgggtttcgctcagctattagcgacccgcaatctctgaccattattggatttggtgcgttgttaaccttgtttattagtattaccgtcttgttcgttgggcataagttgatgaaaatcccgtttggggaaacggcgggtatcttagctggaacgcagacccatccagcagtattatcatatgtgtctgacgcatctcgcaacgagttgccagccatggggtacacctcagtgtatcccttggctatgattgcgaaaatcctggctgcacaaacacttttgtttctgttgatt

SEQ ID NO: 47 - DNA, 2660 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacccggggcccaataggctccctataagagatagaactatgctgacattcattgaactccttattggggttgtggttattgtgggtgtagctcgctacatcattaaagggtattctgccactggcgtgttatttgtcggtggcctgttattgctgattatcagtgccattatggggcacaaagtgttaccgtccagccaggcttcaacaggctacagcgccacggatatcgttgaatacgttaaaatattgctaatgagccgcggcggcgacctcggcatgatgattatgatgctgtgtggctttgccgcttacatgacccatatcggcgcgaatgatatggtggtcaagctggcgtcaaaaccattgcagtatattaactccccttaccttctgatgattgccgcctattttgttgcctgtctgatgtcactggccgtctcttccgcaaccggtctgggtgttttgctgatggcaaccctgtttccggtgatggtaaacgttggtatcagtcgtggcgcagctgctgccatttgtgcctccccggcggcgattattctcgcaccgacttcaggggatgtggtgctggcggcgcaggcttccgaaatgtcgctgattgacttcgccttcaaaacaacgctgcctatctcaattgctgcaattatcggcatggcgatcgcccacttcttctggcaacgttatctggataaaaaagagcacatctctcatgaaatgttagatgtcagtgaaatcaccaccactgcccctgcgttttatgccattttgccgttcacgccgatcatcggagtactgatttttgacggcaaatggggtccgcaattacacatcatcactattctggtgatttgtatgctaattgcctccattctggagttcatccgcagctttaatacccagaaagttttctctggtctggaagtggcttatcgcggtatggcagatgcatttgctaacgtggtgatgctgctggttgccgctggggtattcgctcaggggcttagcaccatcggctttattcaaagtctgatttctatcgctacctcgtttggttcggcgagtatcatcctgatgctggtattggtgatcctgacaatgctggcggcagtcacgaccggttcaggcaatgcgccgttttatgcgtttgttgagatgatcccgaaactggcgcactcctccggcattaacccggcgtatttgactatcccgatgctgcaggcgtcaaacctgggtcgtaccctatcacccgtttctggcgtagtcgttgcggttgccgggatggcgaagatctcgccgtttgaagtcgtaaaacgcacctcggtgccggtgcttgttggtttggtgattgttatcgttgctacagagctgatggtgccaggaacggcagcagcggtcacaggcaagtaaggaatcgactccacgtccctagcgtgtgtaggctggagctgcttcgaagttcctatactttctagagaataggaacttc

SEQ ID NO: 48 - DNA, 1419 bp, Artificial Sequence
  Description of Artificial Sequence Synthetic polynucleotide
gggcccaataggctccctataagagatagaactatgctgacattcattgaactccttattggggttgtggttattgtgggtgtagctcgctacatcattaaagggtattctgccactggcgtgttatttgtcggtggcctgttattgctgattatcagtgccattatggggcacaaagtgttaccgtccagccaggcttcaacaggctacagcgccacggatatcgttgaatacgttaaaatattgctaatgagccgcggcggcgacctcggcatgatgattatgatgctgtgtggctttgccgcttacatgacccatatcggcgcgaatgatatggtggtcaagctggcgtcaaaaccattgcagtatattaactccccttaccttctgatgattgccgcctattttgttgcctgtctgatgtcactggccgtctcttccgcaaccggtctgggtgttttgctgatggcaaccctgtttccggtgatggtaaacgttggtatcagtcgtggcgcagctgctgccatttgtgcctccccggcggcgattattctcgcaccgacttcaggggatgtggtgctggcggcgcaggcttccgaaatgtcgctgattgacttcgccttcaaaacaacgctgcctatctcaattgctgcaattatcggcatggcgatcgcccacttcttctggcaacgttatctggataaaaaagagcacatctctcatgaaatgttagatgtcagtgaaatcaccaccactgcccctgcgttttatgccattttgccgttcacgccgatcatcggagtactgatttttgacggcaaatggggtccgcaattacacatcatcactattctggtgatttgtatgctaattgcctccattctggagttcatccgcagctttaatacccagaaagttttctctggtctggaagtggcttatcgcggtatggcagatgcatttgctaacgtggtgatgctgctggttgccgctggggtattcgctcaggggcttagcaccatcggctttattcaaagtctgatttctatcgctacctcgtttggttcggcgagtatcatcctgatgctggtattggtgatcctgacaatgctggcggcagtcacgaccggttcaggcaatgcgccgttttatgcgtttgttgagatgatcccgaaactggcgcactcctccggcattaacccggcgtatttgactatcccgatgctgcaggcgtcaaacctgggtcgtaccctatcacccgtttctggcgtagtcgttgcggttgccgggatggcgaagatctcgccgtttgaagtcgtaaaacgcacctcggtgccggtgcttgttggtttggtgattgttatcgttgctacagagctgatggtgccaggaacggcagcagcggtcacaggcaagtaa

SEQ ID NO: 49 - DNA, 1386 bp, Artificial Sequence
  Description of Artificial Sequence Synthetic polynucleotide
atgctgacattcattgaactccttattggggttgtggttattgtgggtgtagctcgctacatcattaaagggtattctgccactggcgtgttatttgtcggtggcctgttattgctgattatcagtgccattatggggcacaaagtgttaccgtccagccaggcttcaacaggctacagcgccacggatatcgttgaatacgttaaaatattgctaatgagccgcggcggcgacctcggcatgatgattatgatgctgtgtggctttgccgcttacatgacccatatcggcgcgaatgatatggtggtcaagctggcgtcaaaaccattgcagtatattaactccccttaccttctgatgattgccgcctattttgttgcctgtctgatgtcactggccgtctcttccgcaaccggtctgggtgttttgctgatggcaaccctgtttccggtgatggtaaacgttggtatcagtcgtggcgcagctgctgccatttgtgcctccccggcggcgattattctcgcaccgacttcaggggatgtggtgctggcggcgcaggcttccgaaatgtcgctgattgacttcgccttcaaaacaacgctgcctatctcaattgctgcaattatcggcatggcgatcgcccacttcttctggcaacgttatctggataaaaaagagcacatctctcatgaaatgttagatgtcagtgaaatcaccaccactgcccctgcgttttatgccattttgccgttcacgccgatcatcggagtactgatttttgacggcaaatggggtccgcaattacacatcatcactattctggtgatttgtatgctaattgcctccattctggagttcatccgcagctttaatacccagaaagttttctctggtctggaagtggcttatcgcggtatggcagatgcatttgctaacgtggtgatgctgctggttgccgctggggtattcgctcaggggcttagcaccatcggctttattcaaagtctgatttctatcgctacctcgtttggttcggcgagtatcatcctgatgctggtattggtgatcctgacaatgctggcggcagtcacgaccggttcaggcaatgcgccgttttatgcgtttgttgagatgatcccgaaactggcgcactcctccggcattaacccggcgtatttgactatcccgatgctgcaggcgtcaaacctgggtcgtaccctatcacccgtttctggcgtagtcgttgcggttgccgggatggcgaagatctcgccgtttgaagtcgtaaaacgcacctcggtgccggtgcttgttggtttggtgattgttatcgttgctacagagctgatggtgccaggaacggcagcagcggtcacaggcaagtaa

SEQ ID NO: 50 - DNA, 4305 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacccgtttttttggatggagtgaaacgatgtccttcctggtcgagaatcaattgttagcacttgtcgtgatcatgaccgtcgggcttttacttggacgtatcaaaatctttggtttccgtttgggtgtggccgccgtgttgttcgtcggccttgctttaagcaccattgagcccgacatttcggttccatcccttatttacgtggttggcctttcgctttttgtgtatactatcggtctggaagctggccccggtttttttacatctatgaagacgacgggtttgcgcaataacgcactgacgttaggtgccattatcgcgacaacagcacttgcgtgggcactgattaccgtcttgaatattgatgccgcctcaggagctggtatgcttactggtgccttaactaatacgcccgctatggctgcggtagtggatgcacttccctcattaattgatgacacaggccagctgcatcttattgctgagctgccggtggttgcttattccctggcttatcccttgggggtactgattgtgatcttgagcatcgccatcttttcttcagtgtttaaggttgaccataacaaggaggcagaagaggctggggtagcggtccaagaacttaagggccgccgtatccgcgtaactgtagctgacttgccagcccttgagaacattcctgagttgcttaatttacatgttatcgtctcgcgtgtagagcgcgacggagagcagttcatccccttatatggcgaacatgcacgcatcggcgatgtactgactgtcgtgggggccgacgaggaactgaaccgcgcggaaaaagccatcggagagttaattgacggtgatccttactctaacgttgaactggactatcgtcgtatcttcgtctctaatacggcggttgtcggtacacccctgagcaaattgcaaccgctttttaaagatatgcttattactcgcattcgccgcggtgatacggatctggtagcttcctcggacatgacgcttcaattaggcgaccgcgttcgtgtggttgccccagccgagaaacttcgtgaagcgactcagttgcttggagactcttacaaaaagctgtccgactttaatttattgcctcttgctgcgggcttaatgattggcgtccttgttggaatggttgaattcccactgcctggggggtcatctttaaaacttggcaatgccggtggtccgttggttgtcgcgctgttgcttgggatgatcaatcgtacgggaaagttcgtctggcagatcccgtacggagcaaacttggcgttacgtcagttgggtatcaccctgttcttggcggctattggcacttccgcgggagctgggtttcgctcagctattagcgacccgcaatctctgaccattattggatttggtgcgttgttaaccttgtttattagtattaccgtcttgttcgttgggcataagttgatgaaaatcccgtttggggaaacggcgggtatcttagctggaacgcagacccatccagcagtattatcatatgtgtctgacgcatctcgcaacgagttgccagccatggggtacacctcagtgtatcccttggctatgattgcgaaaatcctggctgcacaaacacttttgtttctgttgatttaatgagggcccaataggctccctataagagatagaactatgctgacattcattgaactccttattggggttgtggttattgtgggtgtagctcgctacatcattaaagggtattctgccactggcgtgttatttgtcggtggcctgttattgctgattatcagtgccattatggggcacaaagtgttaccgtccagccaggcttcaacaggctacagcgccacggatatcgttgaatacgttaaaatattgctaatgagccgcggcggcgacctcggcatgatgattatgatgctgtgtggctttgccgcttacatgacccatatcggcgcgaatgatatggtggtcaagctggcgtcaaaaccattgcagtatattaactccccttaccttctgatgattgccgcctattttgttgcctgtctgatgtcactggccgtctcttccgcaaccggtctgggtgttttgctgatggcaaccctgtttccggtgatggtaaacgttggtatcagtcgtggcgcagctgctgccatttgtgcctccccggcggcgattattctcgcaccgacttcaggggatgtggtgctggcggcgcaggcttccgaaatgtcgctgattgacttcgccttcaaaacaacgctgcctatctcaattgctgcaattatcggcatggcgatcgcccacttcttctggcaacgttatctggataaaaaagagcacatctctcatgaaatgttagatgtcagtgaaatcaccaccactgcccctgcgttttatgccattttgccgttcacgccgatcatcggagtactgatttttgacggcaaatggggtccgcaattacacatcatcactattctggtgatttgtatgctaattgcctccattctggagttcatccgcagctttaatacccagaaagttttctctggtctggaagtggcttatcgcggtatggcagatgcatttgctaacgtggtgatgctgctggttgccgctggggtattcgctcaggggcttagcaccatcggctttattcaaagtctgatttctatcgctacctcgtttggttcggcgagtatcatcctgatgctggtattggtgatcctgacaatgctggcggcagtcacgaccggttcaggcaatgcgccgttttatgcgtttgttgagatgatcccgaaactggcgcactcctccggcattaacccggcgtatttgactatcccgatgctgcaggcgtcaaacctgggtcgtaccctatcacccgtttctggcgtagtcgttgcggttgccgggatggcgaagatctcgccgtttgaagtcgtaaaacgcacctcggtgccggtgcttgttggtttggtgattgttatcgttgctacagagctgatggtgccaggaacggcagcagcggtcacaggcaagtaaggaatcgactccacgtccctagcgtgtgtaggctggagctgcttcgaagttcctatactttctagagaataggaacttc

SEQ ID NO: 51 - DNA, 4226 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacccgtttttttggatggagtgaaacgatgtccttcctggtcgagaatcaattgttagcacttgtcgtgatcatgaccgtcgggcttttacttggacgtatcaaaatctttggtttccgtttgggtgtggccgccgtgttgttcgtcggccttgctttaagcaccattgagcccgacatttcggttccatcccttatttacgtggttggcctttcgctttttgtgtatactatcggtctggaagctggccccggtttttttacatctatgaagacgacgggtttgcgcaataacgcactgacgttaggtgccattatcgcgacaacagcacttgcgtgggcactgattaccgtcttgaatattgatgccgcctcaggagctggtatgcttactggtgccttaactaatacgcccgctatggctgcggtagtggatgcacttccctcattaattgatgacacaggccagctgcatcttattgctgagctgccggtggttgcttattccctggcttatcccttgggggtactgattgtgatcttgagcatcgccatcttttcttcagtgtttaaggttgaccataacaaggaggcagaagaggctggggtagcggtccaagaacttaagggccgccgtatccgcgtaactgtagctgacttgccagcccttgagaacattcctgagttgcttaatttacatgttatcgtctcgcgtgtagagcgcgacggagagcagttcatccccttatatggcgaacatgcacgcatcggcgatgtactgactgtcgtgggggccgacgaggaactgaaccgcgcggaaaaagccatcggagagttaattgacggtgatccttactctaacgttgaactggactatcgtcgtatcttcgtctctaatacggcggttgtcggtacacccctgagcaaattgcaaccgctttttaaagatatgcttattactcgcattcgccgcggtgatacggatctggtagcttcctcggacatgacgcttcaattaggcgaccgcgttcgtgtggttgccccagccgagaaacttcgtgaagcgactcagttgcttggagactcttacaaaaagctgtccgactttaatttattgcctcttgctgcgggcttaatgattggcgtccttgttggaatggttgaattcccactgcctggggggtcatctttaaaacttggcaatgccggtggtccgttggttgtcgcgctgttgcttgggatgatcaatcgtacgggaaagttcgtctggcagatcccgtacggagcaaacttggcgttacgtcagttgggtatcaccctgttcttggcggctattggcacttccgcgggagctgggtttcgctcagctattagcgacccgcaatctctgaccattattggatttggtgcgttgttaaccttgtttattagtattaccgtcttgttcgttgggcataagttgatgaaaatcccgtttggggaaacggcgggtatcttagctggaacgcagacccatccagcagtattatcatatgtgtctgacgcatctcgcaacgagttgccagccatggggtacacctcagtgtatcccttggctatgattgcgaaaatcctggctgcacaaacacttttgtttctgttgatttaatgagggcccaataggctccctataagagatagaactatgctgacattcattgaactccttattggggttgtggttattgtgggtgtagctcgctacatcattaaagggtattctgccactggcgtgttatttgtcggtggcctgttattgctgattatcagtgccattatggggcacaaagtgttaccgtccagccaggcttcaacaggctacagcgccacggatatcgttgaatacgttaaaatattgctaatgagccgcggcggcgacctcggcatgatgattatgatgctgtgtggctttgccgcttacatgacccatatcggcgcgaatgatatggtggtcaagctggcgtcaaaaccattgcagtatattaactccccttaccttctgatgattgccgcctattttgttgcctgtctgatgtcactggccgtctcttccgcaaccggtctgggtgttttgctgatggcaaccctgtttccggtgatggtaaacgttggtatcagtcgtggcgcagctgctgccatttgtgcctccccggcggcgattattctcgcaccgacttcaggggatgtggtgctggcggcgcaggcttccgaaatgtcgctgattgacttcgccttcaaaacaacgctgcctatctcaattgctgcaattatcggcatggcgatcgcccacttcttctggcaacgttatctggataaaaaagagcacatctctcatgaaatgttagatgtcagtgaaatcaccaccactgcccctgcgttttatgccattttgccgttcacgccgatcatcggagtactgatttttgacggcaaatggggtccgcaattacacatcatcactattctggtgatttgtatgctaattgcctccattctggagttcatccgcagctttaatacccagaaagttttctctggtctggaagtggcttatcgcggtatggcagatgcatttgctaacgtggtgatgctgctggttgccgctggggtattcgctcaggggcttagcaccatcggctttattcaaagtctgatttctatcgctacctcgtttggttcggcgagtatcatcctgatgctggtattggtgatcctgacaatgctggcggcagtcacgaccggttcaggcaatgcgccgttttatgcgtttgttgagatgatcccgaaactggcgcactcctccggcattaacccggcgtatttgactatcccgatgctgcaggcgtcaaacctgggtcgtaccctatcacccgtttctggcgtagtcgttgcggttgccgggatggcgaagatctcgccgtttgaagtcgtaaaacgcacctcggtgccggtgcttgttggtttggtgattgttatcgttgctacagagctgatggtgccaggaacggcagcagcggtcacaggcaagtaa

SEQ ID NO: 52 - DNA, 3068 bp, Artificial Sequence
  Description of Artificial Sequence Synthetic polynucleotide
cccgtttttttggatggagtgaaacgatgtccttcctggtcgagaatcaattgttagcacttgtcgtgatcatgaccgtcgggcttttacttggacgtatcaaaatctttggtttccgtttgggtgtggccgccgtgttgttcgtcggccttgctttaagcaccattgagcccgacatttcggttccatcccttatttacgtggttggcctttcgctttttgtgtatactatcggtctggaagctggccccggtttttttacatctatgaagacgacgggtttgcgcaataacgcactgacgttaggtgccattatcgcgacaacagcacttgcgtgggcactgattaccgtcttgaatattgatgccgcctcaggagctggtatgcttactggtgccttaactaatacgcccgctatggctgcggtagtggatgcacttccctcattaattgatgacacaggccagctgcatcttattgctgagctgccggtggttgcttattccctggcttatcccttgggggtactgattgtgatcttgagcatcgccatcttttcttcagtgtttaaggttgaccataacaaggaggcagaagaggctggggtagcggtccaagaacttaagggccgccgtatccgcgtaactgtagctgacttgccagcccttgagaacattcctgagttgcttaatttacatgttatcgtctcgcgtgtagagcgcgacggagagcagttcatccccttatatggcgaacatgcacgcatcggcgatgtactgactgtcgtgggggccgacgaggaactgaaccgcgcggaaaaagccatcggagagttaattgacggtgatccttactctaacgttgaactggactatcgtcgtatcttcgtctctaatacggcggttgtcggtacacccctgagcaaattgcaaccgctttttaaagatatgcttattactcgcattcgccgcggtgatacggatctggtagcttcctcggacatgacgcttcaattaggcgaccgcgttcgtgtggttgccccagccgagaaacttcgtgaagcgactcagttgcttggagactcttacaaaaagctgtccgactttaatttattgcctcttgctgcgggcttaatgattggcgtccttgttggaatggttgaattcccactgcctggggggtcatctttaaaacttggcaatgccggtggtccgttggttgtcgcgctgttgcttgggatgatcaatcgtacgggaaagttcgtctggcagatcccgtacggagcaaacttggcgttacgtcagttgggtatcaccctgttcttggcggctattggcacttccgcgggagctgggtttcgctcagctattagcgacccgcaatctctgaccattattggatttggtgcgttgttaaccttgtttattagtattaccgtcttgttcgttgggcataagttgatgaaaatcccgtttggggaaacggcgggtatcttagctggaacgcagacccatccagcagtattatcatatgtgtctgacgcatctcgcaacgagttgccagccatggggtacacctcagtgtatcccttggctatgattgcgaaaatcctggctgcacaaacacttttgtttctgttgatttaatgagggcccaataggctccctataagagatagaactatgctgacattcattgaactccttattggggttgtggttattgtgggtgtagctcgctacatcattaaagggtattctgccactggcgtgttatttgtcggtggcctgttattgctgattatcagtgccattatggggcacaaagtgttaccgtccagccaggcttcaacaggctacagcgccacggatatcgttgaatacgttaaaatattgctaatgagccgcggcggcgacctcggcatgatgattatgatgctgtgtggctttgccgcttacatgacccatatcggcgcgaatgatatggtggtcaagctggcgtcaaaaccattgcagtatattaactccccttaccttctgatgattgccgcctattttgttgcctgtctgatgtcactggccgtctcttccgcaaccggtctgggtgttttgctgatggcaaccctgtttccggtgatggtaaacgttggtatcagtcgtggcgcagctgctgccatttgtgcctccccggcggcgattattctcgcaccgacttcaggggatgtggtgctggcggcgcaggcttccgaaatgtcgctgattgacttcgccttcaaaacaacgctgcctatctcaattgctgcaattatcggcatggcgatcgcccacttcttctggcaacgttatctggataaaaaagagcacatctctcatgaaatgttagatgtcagtgaaatcaccaccactgcccctgcgttttatgccattttgccgttcacgccgatcatcggagtactgatttttgacggcaaatggggtccgcaattacacatcatcactattctggtgatttgtatgctaattgcctccattctggagttcatccgcagctttaatacccagaaagttttctctggtctggaagtggcttatcgcggtatggcagatgcatttgctaacgtggtgatgctgctggttgccgctggggtattcgctcaggggcttagcaccatcggctttattcaaagtctgatttctatcgctacctcgtttggttcggcgagtatcatcctgatgctggtattggtgatcctgacaatgctggcggcagtcacgaccggttcaggcaatgcgccgttttatgcgtttgttgagatgatcccgaaactggcgcactcctccggcattaacccggcgtatttgactatcccgatgctgcaggcgtcaaacctgggtcgtaccctatcacccgtttctggcgtagtcgttgcggttgccgggatggcgaagatctcgccgtttgaagtcgtaaaacgcacctcggtgccggtgcttgttggtttggtgattgttatcgttgctacagagctgatggtgccaggaacggcagcagcggtcacaggcaagtaa

SEQ ID NO: 53 - DNA, 6480 bp, Artificial Sequence
  Description of Artificial Sequence Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaatgtctctacactctccaggtaaagcgtttcgcgctgcacttagcaaagaaaccccgttgcaaattgttggcaccatcaacgctaaccatgcgctgctggcgcagcgtgccggatatcaggcgatttatctctccggcggtggcgtggcggcaggatcgctggggctgcccgatctcggtatttctactcttgatgacgtgctgacagatattcgccgtatcaccgacgtttgttcgctgccgctgctggtggatgcggatatcggttttggttcttcagcctttaacgtggcgcgtacggtgaaatcaatgattaaagccggtgcggcaggattgcatattgaagatcaggttggtgcgaaacgctgcggtcatcgtccgaataaagcgatcgtctcgaaagaagagatggtggatcggatccgcgcggcggtggatgcgaaaaccgatcctgattttgtgatcatggcgcgcaccgatgcgctggcggtagaggggctggatgcggcgatcgagcgtgcgcaggcctatgttgaagcgggtgccgaaatgctgttcccggaggcgattaccgaactcgccatgtatcgccagtttgccgatgcggtgcaggtgccgatcctctccaacattaccgaatttggcgcaacaccgctgtttaccaccgacgaattacgcagcgcccatgtcgcaatggcgctctacccgctttcagcgtttcgcgccatgaaccgcgccgctgaacatgtctataacatcctgcgtcaggaaggcacacagaaaagcgtcatcgacaccatgcagacccgcaacgagctgtacgaaagcatcaactactaccagtacgaagagaagctcgacgacctgtttgcccgtggtcaggtgaaataaaaacgcccgttggttgtattcgacaaccgatgcctgatgcgccgctgacgcgacttatcaggcctacgaggtgaactgaactgtaggtcggataagacgcatagcgtcgcatccgacaacaatctcgaccctacaaatgataacaatgacgaggacaatatgagcgacacaacgatcctgcaaaacagtacccatgtcattaaaccgaaaaaatcggtggcactttccggcgttccggcgggcaatacggcgctctgcaccgtgggtaaaagcggcaacgacctgcattaccgtggctacgatattcttgatctggcggaacattgtgaatttgaagaagtggcgcacctgctgatccacggcaaactgccaacccgtgacgaactcgccgcctacaaaacgaaactgaaagccctgcgtggtttaccggctaacgtgcgtaccgtgctggaagccttaccggcggcgtcacacccgatggatgttatgcgcaccggcgtttccgcgctcggctgcacgctgccagaaaaagaggggcacaccgtttctggtgcgcgggatattgccgacaaactgctggcgtcacttagttcgattcttctctactggtatcactacagccacaacggcgaacgcatccagccggaaactgatgacgactctatcggcggtcacttcctgcatctgctgcacggcgaaaagccgtcgcaaagctgggaaaaggcgatgcatatctcgctggtgctgtacgccgaacacgagtttaacgcttccacctttaccagccgggtgattgcgggcactggctctgatatgtattccgccattattggcgcgattggcgcactgcgcgggccgaaacacggcggggcgaatgaagtgtcgctggagatccagcaacgctacgaaacgccgggcgaagccgaagccgatatccgcaagcgggtggaaaacaaagaagtggtcattggttttgggcatccggtttataccatcgccgacccgcgtcatcaggtgatcaaacgtgtggcgaagcagctctcgcaggaaggcggctcgctgaagatgtacaacatcgccgatcgcctggaaacggtgatgtgggagagcaaaaagatgttccccaatctcgactggttctccgctgtttcctacaacatgatgggtgttcccaccgagatgttcacaccactgtttgttatcgcccgcgtcactggctgggcggcgcacattatcgaacaacgtcaggacaacaaaattatccgtccttccgccaattatgttggaccggaagaccgccagtttgtcgcgctggataagcgccagtaaacctctacgaataacaataaggaaacgtacccaatgtcagctcaaatcaacaacatccgcccggaatttgatcgtgaaatcgttgatatcgtcgattacgtgatgaactacgaaatcagctccagagtagcctacgacaccgctcattactgcctgcttgacacgctcggctgcggtctggaagctctcgaatatccggcctgtaaaaaactgctggggccaattgtccccggcaccgtcgtacccaacggcgtgcgcgttcccggaactcagtttcagctcgaccccgtccaggcggcatttaacattggcgcgatgatccgttggctcgatttcaacgatacctggctggcggcggagtgggggcatccttccgacaacctcggcggcattctggcaacggcggactggctttcgcgcaacgcgatcgccagcggcaaagcgccgttgaccatgaaacaggtgctgaccggaatgatcaaagcccatgaaattcagggctgcatcgcgctggaaaactcctttaaccgcgttggtctcgaccacgttctgttagtgaaagtggcttccaccgccgtggtcgccgaaatgctcggcctgacccgcgaggaaattctcaacgccgtttcgctggcatgggtagacggacagtcgctgcgcacttatcgtcatgcaccgaacaccggtacgcgtaaatcctgggcggcgggcgatgctacatcccgcgcggtacgtctggcgctgatggcgaaaacgggcgaaatgggttacccgtcagccctgaccgcgccggtgtggggtttctacgacgtctcctttaaaggtgagtcattccgcttccagcgtccgtacggttcctacgtcatggaaaatgtgctgttcaaaatctccttcccggcggagttccactcccagacggcagttgaagcggcgatgacgctctatgaacagatgcaggcagcaggcaaaacggcggcagatatcgaaaaagtgaccattcgcacccacgaagcctgtattcgcatcatcgacaaaaaagggccgctcaataacccggcagaccgcgaccactgcattcagtacatggtggcgatcccgctgctgttcggacgcttaacggcggcagattacgaggacaacgttgcgcaagataaacgcatcgacgccctgcgcgagaagatcaattgctttgaagatccggcgtttaccgctgactaccacgacccggaaaaacgcgccatcgccaatgccataacccttgagttcaccgacggcacacgatttgaagaagtggtggtggagtacccaattggtcatgctcgccgccgtcaggatggcattccgaagctggtcgataaattcaaaatcaatctcgcgcgccagttcccgactcgccagcagcagcgcattctggaggtttctctcgacagaactcgcctggaacagatgccggtcaatgagtatctcgacctgtacgtcatttaagtaaacggcggtaaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtaggtcggataaggcgctcgcgccgcatccgacaccgtgcgcagatgcctgatgcgacgctgacgcgtcttatcatgcctcgctctcgagtcccgtcaagtcagcgtaatgctctgccagtgttacaaccaattaaccaattctgat

SEQ ID NO: 54 - DNA, 5709 bp, Artificial Sequence
  Description of Artificial Sequence Synthetic polynucleotide
taattcctaatttttgttgacactctatcattgatagagttattttaccactccctatcagtgatagagaaaagtgaaatgtctctacactctccaggtaaagcgtttcgcgctgcacttagcaaagaaaccccgttgcaaattgttggcaccatcaacgctaaccatgcgctgctggcgcagcgtgccggatatcaggcgatttatctctccggcggtggcgtggcggcaggatcgctggggctgcccgatctcggtatttctactcttgatgacgtgctgacagatattcgccgtatcaccgacgtttgttcgctgccgctgctggtggatgcggatatcggttttggttcttcagcctttaacgtggcgcgtacggtgaaatcaatgattaaagccggtgcggcaggattgcatattgaagatcaggttggtgcgaaacgctgcggtcatcgtccgaataaagcgatcgtctcgaaagaagagatggtggatcggatccgcgcggcggtggatgcgaaaaccgatcctgattttgtgatcatggcgcgcaccgatgcgctggcggtagaggggctggatgcggcgatcgagcgtgcgcaggcctatgttgaagcgggtgccgaaatgctgttcccggaggcgattaccgaactcgccatgtatcgccagtttgccgatgcggtgcaggtgccgatcctctccaacattaccgaatttggcgcaacaccgctgtttaccaccgacgaattacgcagcgcccatgtcgcaatggcgctctacccgctttcagcgtttcgcgccatgaaccgcgccgctgaacatgtctataacatcctgcgtcaggaaggcacacagaaaagcgtcatcgacaccatgcagacccgcaacgagctgtacgaaagcatcaactactaccagtacgaagagaagctcgacgacctgtttgcccgtggtcaggtgaaataaaaacgcccgttggttgtattcgacaaccgatgcctgatgcgccgctgacgcgacttatcaggcctacgaggtgaactgaactgtaggtcggataagacgcatagcgtcgcatccgacaacaatctcgaccctacaaatgataacaatgacgaggacaatatgagcgacacaacgatcctgcaaaacagtacccatgtcattaaaccgaaaaaatcggtggcactttccggcgttccggcgggcaatacggcgctctgcaccgtgggtaaaagcggcaacgacctgcattaccgtggctacgatattcttgatctggcggaacattgtgaatttgaagaagtggcgcacctgctgatccacggcaaactgccaacccgtgacgaactcgccgcctacaaaacgaaactgaaagccctgcgtggtttaccggctaacgtgcgtaccgtgctggaagccttaccggcggcgtcacacccgatggatgttatgcgcaccggcgtttccgcgctcggctgcacgctgccagaaaaagaggggcacaccgtttctggtgcgcgggatattgccgacaaactgctggcgtcacttagttcgattcttctctactggtatcactacagccacaacggcgaacgcatccagccggaaactgatgacgactctatcggcggtcacttcctgcatctgctgcacggcgaaaagccgtcgcaaagctgggaaaaggcgatgcatatctcgctggtgctgtacgccgaacacgagtttaacgcttccacctttaccagccgggtgattgcgggcactggctctgatatgtattccgccattattggcgcgattggcgcactgcgcgggccgaaacacggcggggcgaatgaagtgtcgctggagatccagcaacgctacgaaacgccgggcgaagccgaagccgatatccgcaagcgggtggaaaacaaagaagtggtcattggttttgggcatccggtttataccatcgccgacccgcgtcatcaggtgatcaaacgtgtggcgaagcagctctcgcaggaaggcggctcgctgaagatgtacaacatcgccgatcgcctggaaacggtgatgtgggagagcaaaaagatgttccccaatctcgactggttctccgctgtttcctacaacatgatgggtgttcccaccgagatgttcacaccactgtttgttatcgcccgcgtcactggctgggcggcgcacattatcgaacaacgtcaggacaacaaaattatccgtccttccgccaattatgttggaccggaagaccgccagtttgtcgcgctggataagcgccagtaaacctctacgaataacaataaggaaacgtacccaatgtcagctcaaatcaacaacatccgcccggaatttgatcgtgaaatcgttgatatcgtcgattacgtgatgaactacgaaatcagctccagagtagcctacgacaccgctcattactgcctgcttgacacgctcggctgcggtctggaagctctcgaatatccggcctgtaaaaaactgctggggccaattgtccccggcaccgtcgtacccaacggcgtgcgcgttcccggaactcagtttcagctcgaccccgtccaggcggcatttaacattggcgcgatgatccgttggctcgatttcaacgatacctggctggcggcggagtgggggcatccttccgacaacctcggcggcattctggcaacggcggactggctttcgcgcaacgcgatcgccagcggcaaagcgccgttgaccatgaaacaggtgctgaccggaatgatcaaagcccatgaaattcagggctgcatcgcgctggaaaactcctttaaccgcgttggtctcgaccacgttctgttagtgaaagtggcttccaccgccgtggtcgccgaaatgctcggcctgacccgcgaggaaattctcaacgccgtttcgctggcatgggtagacggacagtcgctgcgcacttatcgtcatgcaccgaacaccggtacgcgtaaatcctgggcggcgggcgatgctacatcccgcgcggtacgtctggcgctgatggcgaaaacgggcgaaatgggttacccgtcagccctgaccgcgccggtgtggggtttctacgacgtctcctttaaaggtgagtcattccgcttccagcgtccgtacggttcctacgtcatggaaaatgtgctgttcaaaatctccttcccggcggagttccactcccagacggcagttgaagcggcgatgacgctctatgaacagatgcaggcagcaggcaaaacggcggcagatatcgaaaaagtgaccattcgcacccacgaagcctgtattcgcatcatcgacaaaaaagggccgctcaataacccggcagaccgcgaccactgcattcagtacatggtggcgatcccgctgctgttcggacgcttaacggcggcagattacgaggacaacgttgcgcaagataaacgcatcgacgccctgcgcgagaagatcaattgctttgaagatccggcgtttaccgctgactaccacgacccggaaaaacgcgccatcgccaatgccataacccttgagttcaccgacggcacacgatttgaagaagtggtggtggagtacccaattggtcatgctcgccgccgtcaggatggcattccgaagctggtcgataaattcaaaatcaatctcgcgcgccagttcccgactcgccagcagcagcgcattctggaggtttctctcgacagaactcgcctggaacagatgccggtcaatgagtatctcgacctgtacgtcatttaagtaaacggcggtaaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtag

SEQ ID NO: 55 - DNA, 5631 bp, Artificial Sequence
  Description of Artificial Sequence Synthetic polynucleotide
atgtctctacactctccaggtaaagcgtttcgcgctgcacttagcaaagaaaccccgttgcaaattgttggcaccatcaacgctaaccatgcgctgctggcgcagcgtgccggatatcaggcgatttatctctccggcggtggcgtggcggcaggatcgctggggctgcccgatctcggtatttctactcttgatgacgtgctgacagatattcgccgtatcaccgacgtttgttcgctgccgctgctggtggatgcggatatcggttttggttcttcagcctttaacgtggcgcgtacggtgaaatcaatgattaaagccggtgcggcaggattgcatattgaagatcaggttggtgcgaaacgctgcggtcatcgtccgaataaagcgatcgtctcgaaagaagagatggtggatcggatccgcgcggcggtggatgcgaaaaccgatcctgattttgtgatcatggcgcgcaccgatgcgctggcggtagaggggctggatgcggcgatcgagcgtgcgcaggcctatgttgaagcgggtgccgaaatgctgttcccggaggcgattaccgaactcgccatgtatcgccagtttgccgatgcggtgcaggtgccgatcctctccaacattaccgaatttggcgcaacaccgctgtttaccaccgacgaattacgcagcgcccatgtcgcaatggcgctctacccgctttcagcgtttcgcgccatgaaccgcgccgctgaacatgtctataacatcctgcgtcaggaaggcacacagaaaagcgtcatcgacaccatgcagacccgcaacgagctgtacgaaagcatcaactactaccagtacgaagagaagctcgacgacctgtttgcccgtggtcaggtgaaataaaaacgcccgttggttgtattcgacaaccgatgcctgatgcgccgctgacgcgacttatcaggcctacgaggtgaactgaactgtaggtcggataagacgcatagcgtcgcatccgacaacaatctcgaccctacaaatgataacaatgacgaggacaatatgagcgacacaacgatcctgcaaaacagtacccatgtcattaaaccgaaaaaatcggtggcactttccggcgttccggcgggcaatacggcgctctgcaccgtgggtaaaagcggcaacgacctgcattaccgtggctacgatattcttgatctggcggaacattgtgaatttgaagaagtggcgcacctgctgatccacggcaaactgccaacccgtgacgaactcgccgcctacaaaacgaaactgaaagccctgcgtggtttaccggctaacgtgcgtaccgtgctggaagccttaccggcggcgtcacacccgatggatgttatgcgcaccggcgtttccgcgctcggctgcacgctgccagaaaaagaggggcacaccgtttctggtgcgcgggatattgccgacaaactgctggcgtcacttagttcgattcttctctactggtatcactacagccacaacggcgaacgcatccagccggaaactgatgacgactctatcggcggtcacttcctgcatctgctgcacggcgaaaagccgtcgcaaagctgggaaaaggcgatgcatatctcgctggtgctgtacgccgaacacgagtttaacgcttccacctttaccagccgggtgattgcgggcactggctctgatatgtattccgccattattggcgcgattggcgcactgcgcgggccgaaacacggcggggcgaatgaagtgtcgctggagatccagcaacgctacgaaacgccgggcgaagccgaagccgatatccgcaagcgggtggaaaacaaagaagtggtcattggttttgggcatccggtttataccatcgccgacccgcgtcatcaggtgatcaaacgtgtggcgaagcagctctcgcaggaaggcggctcgctgaagatgtacaacatcgccgatcgcctggaaacggtgatgtgggagagcaaaaagatgttccccaatctcgactggttctccgctgtttcctacaacatgatgggtgttcccaccgagatgttcacaccactgtttgttatcgcccgcgtcactggctgggcggcgcacattatcgaacaacgtcaggacaacaaaattatccgtccttccgccaattatgttggaccggaagaccgccagtttgtcgcgctggataagcgccagtaaacctctacgaataacaataaggaaacgtacccaatgtcagctcaaatcaacaacatccgcccggaatttgatcgtgaaatcgttgatatcgtcgattacgtgatgaactacgaaatcagctccagagtagcctacgacaccgctcattactgcctgcttgacacgctcggctgcggtctggaagctctcgaatatccggcctgtaaaaaactgctggggccaattgtccccggcaccgtcgtacccaacggcgtgcgcgttcccggaactcagtttcagctcgaccccgtccaggcggcatttaacattggcgcgatgatccgttggctcgatttcaacgatacctggctggcggcggagtgggggcatccttccgacaacctcggcggcattctggcaacggcggactggctttcgcgcaacgcgatcgccagcggcaaagcgccgttgaccatgaaacaggtgctgaccggaatgatcaaagcccatgaaattcagggctgcatcgcgctggaaaactcctttaaccgcgttggtctcgaccacgttctgttagtgaaagtggcttccaccgccgtggtcgccgaaatgctcggcctgacccgcgaggaaattctcaacgccgtttcgctggcatgggtagacggacagtcgctgcgcacttatcgtcatgcaccgaacaccggtacgcgtaaatcctgggcggcgggcgatgctacatcccgcgcggtacgtctggcgctgatggcgaaaacgggcgaaatgggttacccgtcagccctgaccgcgccggtgtggggtttctacgacgtctcctttaaaggtgagtcattccgcttccagcgtccgtacggttcctacgtcatggaaaatgtgctgttcaaaatctccttcccggcggagttccactcccagacggcagttgaagcggcgatgacgctctatgaacagatgcaggcagcaggcaaaacggcggcagatatcgaaaaagtgaccattcgcacccacgaagcctgtattcgcatcatcgacaaaaaagggccgctcaataacccggcagaccgcgaccactgcattcagtacatggtggcgatcccgctgctgttcggacgcttaacggcggcagattacgaggacaacgttgcgcaagataaacgcatcgacgccctgcgcgagaagatcaattgctttgaagatccggcgtttaccgctgactaccacgacccggaaaaacgcgccatcgccaatgccataacccttgagttcaccgacggcacacgatttgaagaagtggtggtggagtacccaattggtcatgctcgccgccgtcaggatggcattccgaagctggtcgataaattcaaaatcaatctcgcgcgccagttcccgactcgccagcagcagcgcattctggaggtttctctcgacagaactcgcctggaacagatgccggtcaatgagtatctcgacctgtacgtcatttaagtaaacggcggtaaggcgtaagttcaacaggagagcattatgtcttttagcgaattttatcagcgttcgattaacgaaccggagaagttctgggccgagcaggcccggcgtattgactggcagacgccctttacgcaaacgctcgaccacagcaacccgccgtttgcccgttggttttgtgaaggccgaaccaacttgtgtcacaacgctatcgaccgctggctggagaaacagccagaggcgctggcattgattgccgtctcttcggaaacagaggaagagcgtacctttaccttccgccagttacatgacgaagtgaatgcggtggcgtcaatgctgcgctcactgggcgtgcagcgtggcgatcgggtgctggtgtatatgccgatgattgccgaagcgcatattaccctgctggcctgcgcgcgcattggtgctattcactcggtggtgtttgggggatttgcttcgcacagcgtggcaacgcgaattgatgacgctaaaccggtgctgattgtctcggctgatgccggggcgcgcggcggtaaaatcattccgtataaaaaattgctcgacgatgcgataagtcaggcacagcatcagccgcgtcacgttttactggtggatcgcgggctggcgaaaatggcgcgcgttagcgggcgggatgtcgatttcgcgtcgttgcgccatcaacacatcggcgcgcgggtgccggtggcatggctggaatccaacgaaacctcctgcattctctacacctccggcacgaccggcaaacctaaaggtgtgcagcgtgatgtcggcggatatgcggtggcgctggcgacctcgatggacaccatttttggcggcaaagcgggcggcgtgttcttttgtgcttcggatatcggctgggtggtagggcattcgtatatcgtttacgcgccgctgctggcggggatggcgactatcgtttacgaaggattgccgacctggccggactgcggcgtgtggtggaaaattgtcgagaaatatcaggttagccgcatgttctcagcgccgaccgccattcgcgtgctgaaaaaattccctaccgctgaaattcgcaaacacgatctttcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggatgacagaccgacgcgtctgggaagccccggcgtgccgatgtatggctataacgtgcagttgctcaatgaagtcaccggcgaaccgtgtggcgtcaatgagaaagggatgctggtagtggaggggccattgccgccaggctgtattcaaaccatctggggcgacgacgaccgctttgtgaagacgtactggtcgctgttttcccgtccggtgtacgccacttttgactggggcatccgcgatgctgacggttatcactttattctcgggcgcactgacgatgtgattaacgttgccggacatcggctgggtacgcgtgagattgaagagagtatctccagtcatccgggcgttgccgaagtggcggtggttggggtgaaagatgcgctgaaagggcaggtggcggtggcgtttgtcattccgaaagagagcgacagtctggaagaccgtgaggtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggggatctgacgaccattgatgatccggcgtcgttggatcagatccgccaggcgatggaagagtag

SEQ ID NO: 56 - DNA, 891 bp, Artificial Sequence
  Description of Artificial Sequence Synthetic polynucleotide
atgtctctacactctccaggtaaagcgtttcgcgctgcacttagcaaagaaaccccgttgcaaattgttggcaccatcaacgctaaccatgcgctgctggcgcagcgtgccggatatcaggcgatttatctctccggcggtggcgtggcggcaggatcgctggggctgcccgatctcggtatttctactcttgatgacgtgctgacagatattcgccgtatcaccgacgtttgttcgctgccgctgctggtggatgcggatatcggttttggttcttcagcctttaacgtggcgcgtacggtgaaatcaatgattaaagccggtgcggcaggattgcatattgaagatcaggttggtgcgaaacgctgcggtcatcgtccgaataaagcgatcgtctcgaaagaagagatggtggatcggatccgcgcggcggtggatgcgaaaaccgatcctgattttgtgatcatggcgcgcaccgatgcgctggcggtagaggggctggatgcggcgatcgagcgtgcgcaggcctatgttgaagcgggtgccgaaatgctgttcccggaggcgattaccgaactcgccatgtatcgccagtttgccgatgcggtgcaggtgccgatcctctccaacattaccgaatttggcgcaacaccgctgtttaccaccgacgaattacgcagcgcccatgtcgcaatggcgctctacccgctttcagcgtttcgcgccatgaaccgcgccgctgaacatgtctataacatcctgcgtcaggaaggcacacagaaaagcgtcatcgacaccatgcagacccgcaacgagctgtacgaaagcatcaactactaccagtacgaagagaagctcgacgacctgtttgcccgtggtcaggtgaaataa

SEQ ID NO: 57 - DNA, 1170 bp, Artificial Sequence
  Description of Artificial Sequence Synthetic polynucleotide
atgagcgacacaacgatcctgcaaaacagtacccatgtcattaaaccgaaaaaatcggtggcactttccggcgttccggcgggcaatacggcgctctgcaccgtgggtaaaagcggcaacgacctgcattaccgtggctacgatattcttgatctggcggaacattgtgaatttgaagaagtggcgcacctgctgatccacggcaaactgccaacccgtgacgaactcgccgcctacaaaacgaaactgaaagccctgcgtggtttaccggctaacgtgcgtaccgtgctggaagccttaccggcggcgtcacacccgatggatgttatgcgcaccggcgtttccgcgctcggctgcacgctgccagaaaaagaggggcacaccgtttctggtgcgcgggatattgccgacaaactgctggcgtcacttagttcgattcttctctactggtatcactacagccacaacggcgaacgcatccagccggaaactgatgacgactctatcggcggtcacttcctgcatctgctgcacggcgaaaagccgtcgcaaagctgggaaaaggcgatgcatatctcgctggtgctgtacgccgaacacgagtttaacgcttccacctttaccagccgggtgattgcgggcactggctctgatatgtattccgccattattggcgcgattggcgcactgcgcgggccgaaacacggcggggcgaatgaagtgtcgctggagatccagcaacgctacgaaacgccgggcgaagccgaagccgatatccgcaagcgggtggaaaacaaagaagtggtcattggttttgggcatccggtttataccatcgccgacccgcgtcatcaggtgatcaaacgtgtggcgaagcagctctcgcaggaaggcggctcgctgaagatgtacaacatcgccgatcgcctggaaacggtgatgtgggagagcaaaaagatgttccccaatctcgactggttctccgctgtttcctacaacatgatgggtgttcccaccgagatgttcacaccactgtttgttatcgcccgcgtcactggctgggcggcgcacattatcgaacaacgtcaggacaacaaaattatccgtccttccgccaattatgttggaccggaagaccgccagtttgtcgcgctggataagcgccagtaa

SEQ ID NO: 58 - DNA, 1452 bp, Artificial Sequence
  Description of Artificial Sequence Synthetic polynucleotide
atgtcagctcaaatcaacaacatccgcccggaatttgatcgtgaaatcgttgatatcgtcgattacgtgatgaactacgaaatcagctccagagtagcctacgacaccgctcattactgcctgcttgacacgctcggctgcggtctggaagctctcgaatatccggcctgtaaaaaactgctggggccaattgtccccggcaccgtcgtacccaacggcgtgcgcgttcccggaactcagtttcagctcgaccccgtccaggcggcatttaacattggcgcgatgatccgttggctcgatttcaacgatacctggctggcggcggagtgggggcatccttccgacaacctcggcggcattctggcaacggcggactggctttcgcgcaacgcgatcgccagcggcaaagcgccgttgaccatgaaacaggtgctgaccggaatgatcaaagcccatgaaattcagggctgcatcgcgctggaaaactcctttaaccgcgttggtctcgaccacgttctgttagtgaaagtggcttccaccgccgtggtcgccgaaatgctcggcctgacccgcgaggaaattctcaacgccgtttcgctggcatgggtagacggacagtcgctgcgcacttatcgtcatgcaccgaacaccggtacgcgtaaatcctgggcggcgggcgatgctacatcccgcgcggtacgtctggcgctgatggcgaaaacgggcgaaatgggttacccgtcagccctgaccgcgccggtgtggggtttctacgacgtctcctttaaaggtgagtcattccgcttccagcgtccgtacggttcctacgtcatggaaaatgtgctgttcaaaatctccttcccggcggagttccactcccagacggcagttgaagcggcgatgacgctctatgaacagatgcaggcagcaggcaaaacggcggcagatatcgaaaaagtgaccattcgcacccacgaagcctgtattcgcatcatcgacaaaaaagggccgctcaataacccggcagaccgcgaccactgcattcagtacatggtggcgatcccgctgctgttcggacgcttaacggcggcagattacgaggacaacgttgcgcaagataaacgcatcgacgccctgcgcgagaagatcaattgctttgaagatccggcgtttaccgctgactaccacgacccggaaaaacgcgccatcgccaatgccataacccttgagttcaccgacggcacacgatttgaagaagtggtggtggagtacccaattggtcatgctcgccgccgtcaggatggcattccgaagctggtcgataaattcaaaatcaatctcgcgcgccagttcccgactcgccagcagcagcgcattctggaggtttctctcgacagaactcgcctggaacagatgccggtcaatgagtatctcgacctgtacgtcatttaa

SEQ ID NO: 59 - DNA, 60 bp, Artificial Sequence
  Description of Artificial Sequence Synthetic primer
tagaactgatgcaaaaagtgctcgacgaaggcacacagatgtgtaggctggagctgcttc

SEQ ID NO: 60 - DNA, 60 bp, Artificial Sequence
  Description of Artificial Sequence Synthetic primer
gtttcgtaattagatagccaccggcgctttaatgcccggacatatgaatatcctccttag

SEQ ID NO: 61 - DNA, 52 bp, Artificial Sequence
  Description of Artificial Sequence Synthetic primer
caacacgtttcctgaggaaccatgaaacagtatttagaactgatgcaaaaag

SEQ ID NO: 62 - DNA, 46 bp, Artificial Sequence
  Description of Artificial Sequence Synthetic primer
cgcacactggcgtcggctctggcaggatgtttcgtaattagatagc

SEQ ID NO: 63 - DNA, 36 bp, Artificial Sequence
  Description of Artificial Sequence Synthetic primer
atatcgtcgcagcccacagcaacacgtttcctgagg

SEQ ID NO: 64 - DNA, 47 bp, Artificial Sequence
  Description of Artificial Sequence Synthetic primer
aagaatttaacggagggcaaaaaaaaccgacgcacactggcgtcggc

SEQ ID NO: 65 - DNA, 3383 bp, Artificial Sequence
  Description of Artificial Sequence Synthetic polynucleotide
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaaatgagcaaagtcagactcgcaattatggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 66 - DNA, 3258 bp, Artificial Sequence
  Description of Artificial Sequence Synthetic polynucleotide
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacctatgacgacctacgatcgggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 67 - DNA, 3386 bp, Artificial Sequence
  Description of Artificial Sequence Synthetic polynucleotide
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 68 - DNA, 3261 bp, Artificial Sequence
  Description of Artificial Sequence Synthetic polynucleotide
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 69 - DNA, 3279 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacatatgctatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 70 - DNA, 1921 bp, Artificial Sequence
  Description of Artificial Sequence Synthetic polynucleotide
ttacccgtctggattttcagtacgcgcttttaaacgacgccacagcgtggtacggctgatccccaaataacgtgcggcggcgcgcttatcgccattaaagcgtgcgagcacctcctgcaatggaagcgcttctgctgacgagggcgtgatttctgctgtggtccccaccagttcaggtaataattgccgcataaattgtctgtccagtgttggtgcgggatcgacgcttaaaaaaagcgccaggcgttccatcatattccgcagttcgcgaatattaccgggccaatgatagttcagtagaagcggctgacactgcgtcagcccatgacgcaccgattcggtaaaagggatctccatcgcggccagcgattgttttaaaaagttttccgccagaggcagaatatcaggctgtcgctcgcgcaaggggggaagcggcagacgcagaatgctcaaacggtaaaacagatcggtacgaaaacgtccttgcgttatctcccgatccagatcgcaatgcgtggcgctgatcacccggacatctaccgggatcggctgatgcccgccaacgcgggtgacggctttttcctccagtacgcgtagaaggcgggtttgtaacggcagcggcatttcgccaatttcgtcaagaaacagcgtgccgccgtgggcgacctcaaacagccccgcacgtccacctcgtcttgagccggtaaacgctccctcctcatagccaaacagttcagcctccagcaacgactcggtaatcgcgccgcaattaacggcgacaaagggcggagaaggcttgttctgacggtggggctgacggttaaacaacgcctgatgaatcgcttgcgccgccagctctttcccggtccctgtttccccctgaatcagcactgccgcgcgggaacgggcatagagtgtaatcgtatggcgaacctgctccatttgtggtgaatcgccgaggatatcgctcagcgcataacgggtctgtaatcccttgctggaggtatgctggctatactgacgccgtgtcaggcgggtcatatccagcgcatcatggaaagcctgacgtacggtggccgctgaataaataaagatggcggtcattcctgcctcttccgccaggtcggtaattagtcctgccccaattacagcctcaatgccgttagctttgagctcgttaatttgcccgcgagcatcctcttcagtgatatagcttcgctgttcaagacggaggtgaaacgttttctgaaaggcgaccagagccggaatggtctcctgataggtcacgattcccattgaggaagtcagctttcccgcttttgccagagcctgtaatacatcgaatccgctgggtttgatgaggatgacaggtaccgacagtcggctttttaaataagcgccgttggaacctgccgcgataatcgcgtcgcagcgttcggttgccagttttttgcgaatgtaggctactgccttttcaaaaccgagctgaataggcgtgatcgtcgccagatgatcaaactccaggctgatatcccgaaatagttcgaacaggcgcgttaccgagaccgtccagatcaccggtttatcgctattatcgcgcgaagcgctatgcacagtaaccatcgtcgtagattcatgtttaaggaacgaattcttgttttatagatgtttcgttaatgttgcaatgaaacacaggcctccgtttcatgaaacgttagctgactcgtttttcttgtgactcgtctgtcagtattaaaaaagatttttcatttaactgattgtttttaaattgaattttatttaatggtttctcggtttttgggtctggcatatcccttgctttaatgagtgcatcttaattaacaattcaataacaagagggctgaatagtaatttcaacaaaataacgagcattcgaatg

SEQ ID NO: 71 - protein, 628 aa, Artificial Sequence
  Description of Artificial Sequence Synthetic polypeptide
MSFSEFYQRSINEPEKFWAEQARRIDWQTPFTQTLDHSNPPFARWFCEGRTNLCHNAIDRWLEKQPEALALIAVSSETEEERTFTFRQLHDEVNAVASMLRSLGVQRGDRVLVYMPMIAEAHITLLACARIGAIHSVVFGGFASHSVATRIDDAKPVLIVSADAGARGGKIIPYKKLLDDAISQAQHQPRHVLLVDRGLAKMARVSGRDVDFASLRHQHIGARVPVAWLESNETSCILYTSGTTGKPKGVQRDVGGYAVALATSMDTIFGGKAGGVFFCASDIGWVVGHSYIVYAPLLAGMATIVYEGLPTWPDCGVWWKIVEKYQVSRMFSAPTAIRVLKKFPTAEIRKHDLSSLEVLYLAGEPLDEPTASWVSNTLDVPVIDNYWQTESGWPIMAIARGLDDRPTRLGSPGVPMYGYNVQLLNEVTGEPCGVNEKGMLVVEGPLPPGCIQTIWGDDDRFVKTYWSLFSRPVYATFDWGIRDADGYHFILGRTDDVINVAGHRLGTREIEESISSHPGVAEVAVVGVKDALKGQVAVAFVIPKESDSLEDREVAHSQEKAIMALVDSQIGNFGRPAHVWFVSQLPKTRSGKMLRRTIQAICEGRDPGDLTTIDDPASLDQIRQAMEE

SEQ ID NO: 72 - protein, 628 aa, Artificial Sequence
  Description of Artificial Sequence Synthetic polypeptide
MSFSEFYQRSINEPEQFWAEQARRIDWQQPFTQTLDYSNPPFARWFCGGTTNLCHNAIDRWLDTQPDALALIAVSSETEEERTFTFRQLYDEVNVVASMLLSLGVRRGDRVLVYMPMIAEAHITLLACARIGAIHSVVFGGFASHSVAARIDDARPVLIVSADAGARGGKVIPYKKLLDEAVDQAQHQPKHVLLVDRGLAKMARVAGRDVDFATLREHHAGARVPVAWLESNESSCILYTSGTTGKPKGVQRDVGGYAVALATSMDTLFGGKAGGVFFCASDIGWVVGHSYIVYAPLLAGMATIVYEGLPTYPDCGVWWKIVEKYRVSRMFSAPTAIRVLKKFPTAQIRNHDLSSLEVLYLAGEPLDEPTAAWVSGTLGVPVIDNYWQTESGWPIMALARTLDDRPSRLGSPGVPMYGYNVQLLNEVTGEPCGANEKGMVVIEGPLPPGCIQTIWGDDARFVNTYWSLFTRQVYATFDWGIRDADGYYFILGRTDDVINVAGHRLGTREIEESISSYPNVAEVAVVGVKDALKGQVAVAFVIPKQSDSLEDREVAHSEEKAIMALVDSQIGNFGRPAHVWFVSQLPKTRSGKMLRRTIQAICEGRDPGDLTTIDDPTSLQQIRQVIEE

SEQ ID NO: 73 - DNA, 1887 bp, Artificial Sequence
  Description of Artificial Sequence Synthetic polynucleotide
atgtcttttagcgaattttatcagcgttcgattaacgaaccggagcagttctgggctgaacaggcccggcgtatcgactggcagcagccgtttacgcagacgctggactacagcaacccgccgtttgcccgctggttttgcggcggcaccactaatctgtgccataacgcgattgaccgctggctggatacccagccggatgcgctggcgctgattgcggtttcctctgagaccgaagaagaacgtaccttcacctttcgtcaactgtatgacgaggtgaatgtcgtggcctctatgctgctgtcactgggcgtgcggcgtggcgatcgggtactggtgtatatgccgatgattgccgaggcgcacatcacattactggcctgcgcgcgcattggcgcgatccattcagtggtgtttggtggttttgcctcgcacagtgtagccgcgcgcatcgacgatgccagaccggtgctgattgtctcggcggacgccggagcgcgaggtgggaaggtcattccctataaaaagcttcttgatgaggcggtcgatcaggcacagcatcagccgaagcatgtactgctggtggatcgggggctggcgaaaatggcgcgggttgccgggcgcgatgtggattttgcgaccctgcgcgaacaccatgccggggcgcgtgtgccagtggcctggcttgaatctaatgaaagttcctgcattctttatacctccggcactaccggcaaaccgaaaggcgttcagcgtgacgttggtggctacgccgtggcgctggcgacatcgatggacaccctctttggcggcaaagcgggcggcgtctttttctgcgcttcggatatcggttgggtagtggggcactcttatattgtgtatgcgccgctgctggcgggtatggcgaccatcgtttatgaaggattgccgacgtatccggactgcggcgtatggtggaaaattgtcgagaaatatcgggtgagccggatgttttcagcgccaaccgccattcgtgtgctgaagaaatttcccaccgcgcagatacgcaatcatgatctctcctcgctggaagttctctatctggcaggcgagccgctcgacgagccaacggcagcctgggttagcggaacactgggtgtgccggtgatcgacaattactggcagaccgaatccggctggccgattatggcgctggcgcgcacgcttgatgacagaccatcgcgtttgggcagtcccggcgtgccgatgtacggctataatgttcaactgctcaacgaggtgaccggtgaaccctgtggtgcgaacgaaaagggaatggtggttattgaagggccgctgccgccgggctgcattcagaccatctggggcgatgacgcacgctttgtgaatacctactggtcactgtttactcgtcaggtgtatgccacctttgactgggggatccgcgacgccgacggctattattttatccttgggcgcacggatgatgtgatcaacgtcgccggacatcgtctcggcacccgtgagatagaggagagcatctccagctatcccaacgttgcggaagtggcggtggtaggggtaaaagacgcgctgaaagggcaggtagcggtagccttcgtgatcccgaaacagagtgacagtctggaagaccgcgaagtggcgcattcggaagagaaggcgattatggcgctggtcgatagtcagatcggcaactttggccgcccggcgcacgtgtggtttgtctcgcagctaccaaaaacccgatccgggaagatgctcagacgaacgatccaggcgatctgcgagggccgggatccaggcgatctgacgaccattgacgatccgacgtcgttgcaacaaattcgccaggtcattgaggagtaa

SEQ ID NO: 74 - protein, 389 aa, Artificial Sequence
  Description of Artificial Sequence Synthetic polypeptide
MSDTTILQNSTHVIKPKKSVALSGVPAGNTALCTVGKSGNDLHYRGYDILDLAEHCEFEEVAHLLIHGKLPTRDELAAYKTKLKALRGLPANVRTVLEALPAASHPMDVMRTGVSALGCTLPEKEGHTVSGARDIADKLLASLSSILLYWYHYSHNGERIQPETDDDSIGGHFLHLLHGEKPSQSWEKAMHISLVLYAEHEFNASTFTSRVIAGTGSDMYSAIIGAIGALRGPKHGGANEVSLEIQQRYETPGEAEADIRKRVENKEVVIGFGHPVYTIADPRHQVIKRVAKQLSQEGGSLKMYNIADRLETVMWESKKMFPNLDWFSAVSYNMMGVPTEMFTPLFVIARVTGWAAHIIEQRQDNKIIRPSANYVGPEDRQFVALDKRQ

SEQ ID NO: 75 - protein, 389 aa, Artificial Sequence
  Description of Artificial Sequence Synthetic polypeptide
MSDTTILQNNTNVIKPKKSVALSGVPAGNTALCTVGKSGNDLHYRGYDILDLAEHCEFEEVAHLLIHGKLPTRDELNAYKSKLKALRGLPANVRTVLEALPAASHPMDVMRTGVSALGCTLPEKEGHTVSGARDIADKLLASLSSILLYWYHYSHNGERIQPETDDDSIGGHFLHLLHGEKPSQSWEKAMHISLVLYAEHEFNASTFTSRVVAGTGSDMYSAIIGAIGALRGPKHGGANEVSLEIQQRYETPDEAEADIRKRIANKEVVIGFGHPVYTIADPRHQVIKRVAKQLSQEGGSLKMYNIADRLETVMWDSKKMFPNLDWFSAVSYNMMGVPTEMFTPLFVIARVTGWAAHIIEQRQDNKIIRPSANYIGPEDRAFTPLEQRQ

SEQ ID NO: 76 - DNA, 1170 bp, Artificial Sequence
  Description of Artificial Sequence Synthetic polynucleotide
atgagcgacacgacgatcctgcaaaacaacacaaatgtcattaagccaaaaaaatccgtcgcattatccggcgtacccgccggaaataccgccttatgcaccgtaggtaaaagcggtaacgatctgcactatcgcgggtacgatattctcgatctcgcggagcactgtgaatttgaagaagttgcgcatctgctcattcacggcaagctgcccacccgtgatgagctgaatgcctataaaagcaaattaaaagcgctgcgtggcttacccgctaacgtccgtaccgtgctggaagcgctgccagcggcatcgcacccgatggacgtaatgcgcaccggcgtttctgcgctgggctgcaccctgccggaaaaagaggggcataccgtttctggcgcgcgtgatatcgccgacaagctgctggcctccctcagctccattctcctttactggtatcactacagccacaacggcgaacgcattcagccagaaactgacgatgactctatcggcgggcatttcctgcatttattacacggcgaaaagccatcgcaaagctgggaaaaggcgatgcacatttcactggtactgtacgccgaacatgagttcaacgcctcaacctttaccagccgggtggtagccggtacgggatcggatatgtactccgccatcattggcgcgataggcgcgcttcgcgggccgaagcacggcggggcgaatgaagtctcgctggagattcagcagcgctacgaaacgccggatgaagcagaagccgatatccgtaaacgtatcgccaataaagaagtggtgattggttttggtcatccggtatacaccatcgccgatccgcgccatcaggtgattaagcgggtagcgaagcagctttcacaggagggcggttcgctgaagatgtacaacattgccgatcggctggagacggtaatgtgggacagcaaaaagatgttccctaatctcgactggttctcggcggtctcctacaacatgatgggcgttcccaccgaaatgtttaccccgctgtttgtgattgcccgcgttacaggttgggcggcgcacatcatcgagcaacgacaggacaacaaaattatccgtccttccgccaattatattggcccggaagatcgcgcctttacgccgctggaacagcgtcagtaa

SEQ ID NO: 77 - protein, 483 aa, Artificial Sequence
  Description of Artificial Sequence Synthetic polypeptide
MSAQINNIRPEFDREIVDIVDYVMNYEISSRVAYDTAHYCLLDTLGCGLEALEYPACKKLLGPIVPGTVVPNGVRVPGTQFQLDPVQAAFNIGAMIRWLDFNDTWLAAEWGHPSDNLGGILATADWLSRNAIASGKAPLTMKQVLTGMIKAHEIQGCIALENSFNRVGLDHVLLVKVASTAVVAEMLGLTREEILNAVSLAWVDGQSLRTYRHAPNTGTRKSWAAGDATSRAVRLALMAKTGEMGYPSALTAPVWGFYDVSFKGESFRFQRPYGSYVMENVLFKISFPAEFHSQTAVEAAMTLYEQMQAAGKTAADIEKVTIRTHEACIRIIDKKGPLNNPADRDHCIQYMVAIPLLFGRLTAADYEDNVAQDKRIDALREKINCFEDPAFTADYHDPEKRAIANAITLEFTDGTRFEEVVVEYPIGHARRRQDGIPKLVDKFKINLARQFPTRQQQRILEVSLDRTRLEQMPVNEYLDLYVI

SEQ ID NO: 78 - protein, 483 aa, Artificial Sequence
  Description of Artificial Sequence Synthetic polypeptide
MSAPVSNVRPEFDREIVDIVDYVMKYNITSKVAYDTAHYCLLDTLGCGLEALEYPACKKLMGPIVPGTVVPNGVRVPGTQFQLDPVQAAFNIGAMIRWLDFNDTWLAAEWGHPSDNLGGILATADWLSRNAVAAGKAPLTMQQVLTGMIKAHEIQGCIALENSFNRVGLDHVLLVKVASTAVVAEMLGLTRDEILNAVSLAWVDGQSLRTYRHAPNTGTRKSWAAGDATSRAVRLALMAKTGEMGYPSALTAKTWGFYDVSFKGEKFRFQRPYGSYVMENVLFKISFPAEFHSQTAVEAAMTLYEQMQAAGKTAADIEKVTIRTHEACIRIIDKKGPLNNPADRDHCIQYMVAIPLLFGRLTAADYEDGVAQDKRIDALREKTHCFEDPAFTTDYHDPEKRSIANAISLEFTDGTRFDEVVVEYPIGHARRRGDGIPKLIEKFKINLARQFPPRQQQRILDVSLDRTRLEQMPVNEYLDLYVI

SEQ ID NO: 79 - DNA, 1452 bp, Artificial Sequence
  Description of Artificial Sequence Synthetic polynucleotide
atgtccgcacctgtttcgaacgtccgccctgaatttgaccgtgaaattgttgatattgttgattatgtgatgaagtacaacatcacctcaaaagtggcttatgacaccgcgcactactgtctgcttgataccctgggctgtgggctggaagcgctggaatatccggcctgtaaaaaattgatggggcctatcgtgccaggtaccgtggtgccgaacggtgtacgtgtaccgggcactcagttccagctcgatccggtgcaggcggcatttaatattggcgcgatgatccgctggctcgactttaacgatacctggcttgccgctgagtggggacacccttccgataacctcggcggtattctggcgaccgccgactggttgtcgcgcaacgccgtcgccgccggtaaagcgccgctgaccatgcagcaggtgctgaccgggatgatcaaagcccacgaaatccagggctgtatcgcgctggaaaactcgtttaaccgcgtgggtctcgatcacgttttgctggtgaaagtggcttccacggctgtagtggctgaaatgctcggcctgacccgcgatgaaattctcaacgccgtatcgctggcgtgggtggatgggcagtcgctgcgtacctatcgccatgcgccaaacaccggtacgcgcaaatcctgggcggcaggcgatgccacttcacgcgcggtgcgtctggcgctgatggcgaaaactggcgagatgggctatccctcggcgttgaccgccaaaacctggggcttttatgacgtctcgttcaaaggcgaaaaattccgtttccagcgcccgtacggctcctacgtgatggaaaacgtgctgttcaaaatctccttcccggcggagttccattcgcagaccgccgttgaagcagcgatgacgctgtatgagcagatgcaggcggctggaaaaacggcggcggatatcgaaaaagtaacgattcgcacccatgaagcctgtatacgcatcattgataaaaaaggcccgctgaataatccggctgaccgcgatcactgtattcagtatatggtggcgatcccactgctgttcggacgcttaacggcggcggattatgaggatggcgtggcgcaggataaacgtattgacgcgctgcgtgaaaaaacgcattgctttgaagacccggcgtttaccactgattatcatgacccggaaaaacgttcgattgccaacgccattagtcttgaatttactgacggtacccgttttgacgaggtggttgtcgagtacccgatcggccacgcgcgtcgtcgcggcgacggcattccaaaacttatcgaaaaatttaaaatcaatctggcgcgccagttcccaccccgccagcaacaacgcatcctggatgtctccctggacagaacgcgcctggagcagatgccggttaatgagtatctcgacttgtacgtcatctag

SEQ ID NO: 80 - protein, 296 aa, Artificial Sequence
  Description of Artificial Sequence Synthetic polypeptide
MSLHSPGKAFRAALSKETPLQIVGTINANHALLAQRAGYQAIYLSGGGVAAGSLGLPDLGISTLDDVLTDIRRITDVCSLPLLVDADIGFGSSAFNVARTVKSMIKAGAAGLHIEDQVGAKRCGHRPNKAIVSKEEMVDRIRAAVDAKTDPDFVIMARTDALAVEGLDAAIERAQAYVEAGAEMLFPEAITELAMYRQFADAVQVPILSNITEFGATPLFTTDELRSAHVAMALYPLSAFRAMNRAAEHVYNILRQEGTQKSVIDTMQTRNELYESINYYQYEEKLDDLFARGQVK

SEQ ID NO: 81 - protein, 294 aa, Artificial Sequence
  Description of Artificial Sequence Synthetic polypeptide
MTLHSPGQAFRAALAKEKPLQIVGAINANHALLAQRAGYQALYLSGGGVAAGSLGLPDLGISTLDDVLTDIRRITDVCPLPLLVDADIGFGSSAFNVARTVKSISKAGAAALHIEDQIGAKRCGHRPNKAIVSKEEMVDRIHAAVDARTDPDFVIMARTDALAVEGLDAAIDRARAYVEAGADMLFPEAITELAMYRQFADAVQVPILANITEFGATPLFTTEELRNANVAMALYPLSAFRAMNRAAEKVYNVLRQEGTQKSVIDIMQTRNELYESINYYQFEEKLDALYAKKS

SEQ ID NO: 82 - DNA, 885 bp, Artificial Sequence
  Description of Artificial Sequence Synthetic polynucleotide
atgacgttacactcaccgggtcaggcgtttcgcgctgcgcttgctaaagaaaaaccattacaaattgtcggcgctatcaacgccaatcatgctctgttagcccagagggctgggtatcaggctctctatctctcgggcggcggtgttgccgcaggctcgctggggctaccggatctgggcatctccacccttgatgacgtattgaccgatatccgccgtatcaccgacgtctgcccgctgccgctgctggtggatgccgatattggcttcggatcgtcggcgtttaacgtagcgcgtaccgtgaaatcgatttccaaagccggcgccgccgcgctgcatattgaagatcagattggcgccaagcgctgcgggcatcggccaaataaagcgatcgtctcgaaagaagagatggtggaccggatccacgcggcggtggatgcgcggaccgatcctgactttgtcattatggcgcgtaccgatgcgctggcggttgaaggccttgatgccgctatcgatcgcgcgcgggcctacgtagaggccggtgccgacatgctgttcccggaggcgattactgaacttgcgatgtaccgccagtttgccgacgcagtgcaggtgccaatccttgccaatattaccgaattcggcgcgacgccgttgtttactaccgaagagctacgcaacgccaacgtggcgatggcgctctatccgctgtcggcgttccgggcgatgaatcgcgcggcggagaaggtttacaacgtgctgcgacaggaaggaacgcaaaagagcgttatcgacatcatgcagacccgtaatgagctgtatgaaagcatcaattattaccagttcgaggaaaaacttgacgcgctgtacgccaaaaaatcgtag

SEQ ID NO: 83 - DNA, 3705 bp, Artificial Sequence
  Description of Artificial Sequence Synthetic polynucleotide
atgtctctacactctccaggtaaagcgtttcgcgctgcacttagcaaagaaaccccgttgcaaattgttggcaccatcaacgctaaccatgcgctgctggcgcagcgtgccggatatcaggcgatttatctctccggcggtggcgtggcggcaggatcgctggggctgcccgatctcggtatttctactcttgatgacgtgctgacagatattcgccgtatcaccgacgtttgttcgctgccgctgctggtggatgcggatatcggttttggttcttcagcctttaacgtggcgcgtacggtgaaatcaatgattaaagccggtgcggcaggattgcatattgaagatcaggttggtgcgaaacgctgcggtcatcgtccgaataaagcgatcgtctcgaaagaagagatggtggatcggatccgcgcggcggtggatgcgaaaaccgatcctgattttgtgatcatggcgcgcaccgatgcgctggcggtagaggggctggatgcggcgatcgagcgtgcgcaggcctatgttgaagcgggtgccgaaatgctgttcccggaggcgattaccgaactcgccatgtatcgccagtttgccgatgcggtgcaggtgccgatcctctccaacattaccgaatttggcgcaacaccgctgtttaccaccgacgaattacgcagcgcccatgtcgcaatggcgctctacccgctttcagcgtttcgcgccatgaaccgcgccgctgaacatgtctataacatcctgcgtcaggaaggcacacagaaaagcgtcatcgacaccatgcagacccgcaacgagctgtacgaaagcatcaactactaccagtacgaagagaagctcgacgacctgtttgcccgtggtcaggtgaaataaaaacgcccgttggttgtattcgacaaccgatgcctgatgcgccgctgacgcgacttatcaggcctacgaggtgaactgaactgtaggtcggataagacgcatagcgtcgcatccgacaacaatctcgaccctacaaatgataacaatgacgaggacaatatgagcgacacaacgatcctgcaaaacagtacccatgtcattaaaccgaaaaaatcggtggcactttccggcgttccggcgggcaatacggcgctctgcaccgtgggtaaaagcggcaacgacctgcattaccgtggctacgatattcttgatctggcggaacattgtgaatttgaagaagtggcgcacctgctgatccacggcaaactgccaacccgtgacgaactcgccgcctacaaaacgaaactgaaagccctgcgtggtttaccggctaacgtgcgtaccgtgctggaagccttaccggcggcgtcacacccgatggatgttatgcgcaccggcgtttccgcgctcggctgcacgctgccagaaaaagaggggcacaccgtttctggtgcgcgggatattgccgacaaactgctggcgtcacttagttcgattcttctctactggtatcactacagccacaacggcgaacgcatccagccggaaactgatgacgactctatcggcggtcacttcctgcatctgctgcacggcgaaaagccgtcgcaaagctgggaaaaggcgatgcatatctcgctggtgctgtacgccgaacacgagtttaacgcttccacctttaccagccgggtgattgcgggcactggctctgatatgtattccgccattattggcgcgattggcgcactgcgcgggccgaaacacggcggggcgaatgaagtgtcgctggagatccagcaacgctacgaaacgccgggcgaagccgaagccgatatccgcaagcgggtggaaaacaaagaagtggtcattggttttgggcatccggtttataccatcgccgacccgcgtcatcaggtgatcaaacgtgtggcgaagcagctctcgcaggaaggcggctcgctgaagatgtacaacatcgccgatcgcctggaaacggtgatgtgggagagcaaaaagatgttccccaatctcgactggttctccgctgtttcctacaacatgatgggtgttcccaccgagatgttcacaccactgtttgttatcgcccgcgtcactggctgggcggcgcacattatcgaacaacgtcaggacaacaaaattatccgtccttccgccaattatgttggaccggaagaccgccagtttgtcgcgctggataagcgccagtaaacctctacgaataacaataaggaaacgtacccaatgtcagctcaaatcaacaacatccgcccggaatttgatcgtgaaatcgttgatatcgtcgattacgtgatgaactacgaaatcagctccagagtagcctacgacaccgctcattactgcctgcttgacacgctcggctgcggtctggaagctctcgaatatccggcctgtaaaaaactgctggggccaattgtccccggcaccgtcgtacccaacggcgtgcgcgttcccggaactcagtttcagctcgaccccgtccaggcggcatttaacattggcgcgatgatccgttggctcgatttcaacgatacctggctggcggcggagtgggggcatccttccgacaacctcggcggcattctggcaacggcggactggctttcgcgcaacgcgatcgccagcggcaaagcgccgttgaccatgaaacaggtgctgaccggaatgatcaaagcccatgaaattcagggctgcatcgcgctggaaaactcctttaaccgcgttggtctcgaccacgttctgttagtgaaagtggcttccaccgccgtggtcgccgaaatgctcggcctgacccgcgaggaaattctcaacgccgtttcgctggcatgggtagacggacagtcgctgcgcacttatcgtcatgcaccgaacaccggtacgcgtaaatcctgggcggcgggcgatgctacatcccgcgcggtacgtctggcgctgatggcgaaaacgggcgaaatgggttacccgtcagccctgaccgcgccggtgtggggtttctacgacgtctcctttaaaggtgagtcattccgcttccagcgtccgtacggttcctacgtcatggaaaatgtgctgttcaaaatctccttcccggcggagttccactcccagacggcagttgaagcggcgatgacgctctatgaacagatgcaggcagcaggcaaaacggcggcagatatcgaaaaagtgaccattcgcacccacgaagcctgtattcgcatcatcgacaaaaaagggccgctcaataacccggcagaccgcgaccactgcattcagtacatggtggcgatcccgctgctgttcggacgcttaacggcggcagattacgaggacaacgttgcgcaagataaacgcatcgacgccctgcgcgagaagatcaattgctttgaagatccggcgtttaccgctgactaccacgacccggaaaaacgcgccatcgccaatgccataacccttgagttcaccgacggcacacgatttgaagaagtggtggtggagtacccaattggtcatgctcgccgccgtcaggatggcattccgaagctggtcgataaattcaaaatcaatctcgcgcgccagttcccgactcgccagcagcagcgcattctggaggtttctctcgacagaactcgcctggaacagatgccggtcaatgagtatctcgacctgtacgtcatttaa

SEQ ID NO: 84 - DNA, 3630 bp, Artificial Sequence
  Description of Artificial Sequence Synthetic polynucleotide
atgacgttacactcaccgggtcaggcgtttcgcgctgcgcttgctaaagaaaaaccattacaaattgtcggcgctatcaacgccaatcatgctctgttagcccagagggctgggtatcaggctctctatctctcgggcggcggtgttgccgcaggctcgctggggctaccggatctgggcatctccacccttgatgacgtattgaccgatatccgccgtatcaccgacgtctgcccgctgccgctgctggtggatgccgatattggcttcggatcgtcggcgtttaacgtagcgcgtaccgtgaaatcgatttccaaagccggcgccgccgcgctgcatattgaagatcagattggcgccaagcgctgcgggcatcggccaaataaagcgatcgtctcgaaagaagagatggtggaccggatccacgcggcggtggatgcgcggaccgatcctgactttgtcattatggcgcgtaccgatgcgctggcggttgaaggccttgatgccgctatcgatcgcgcgcgggcctacgtagaggccggtgccgacatgctgttcccggaggcgattactgaacttgcgatgtaccgccagtttgccgacgcagtgcaggtgccaatccttgccaatattaccgaattcggcgcgacgccgttgtttactaccgaagagctacgcaacgccaacgtggcgatggcgctctatccgctgtcggcgttccgggcgatgaatcgcgcggcggagaaggtttacaacgtgctgcgacaggaaggaacgcaaaagagcgttatcgacatcatgcagacccgtaatgagctgtatgaaagcatcaattattaccagttcgaggaaaaacttgacgcgctgtacgccaaaaaatcgtaggccacgggtctgataaagcgtagccgctatcaagtctgtggcggacaacctcaataccctacacattacaaaaatgacgaggacactatgagcgacacgacgatcctgcaaaacaacacaaatgtcattaagccaaaaaaatccgtcgcattatccggcgtacccgccggaaataccgccttatgcaccgtaggtaaaagcggtaacgatctgcactatcgcgggtacgatattctcgatctcgcggagcactgtgaatttgaagaagttgcgcatctgctcattcacggcaagctgcccacccgtgatgagctgaatgcctataaaagcaaattaaaagcgctgcgtggcttacccgctaacgtccgtaccgtgctggaagcgctgccagcggcatcgcacccgatggacgtaatgcgcaccggcgtttctgcgctgggctgcaccctgccggaaaaagaggggcataccgtttctggcgcgcgtgatatcgccgacaagctgctggcctccctcagctccattctcctttactggtatcactacagccacaacggcgaacgcattcagccagaaactgacgatgactctatcggcgggcatttcctgcatttattacacggcgaaaagccatcgcaaagctgggaaaaggcgatgcacatttcactggtactgtacgccgaacatgagttcaacgcctcaacctttaccagccgggtggtagccggtacgggatcggatatgtactccgccatcattggcgcgataggcgcgcttcgcgggccgaagcacggcggggcgaatgaagtctcgctggagattcagcagcgctacgaaacgccggatgaagcagaagccgatatccgtaaacgtatcgccaataaagaagtggtgattggttttggtcatccggtatacaccatcgccgatccgcgccatcaggtgattaagcgggtagcgaagcagctttcacaggagggcggttcgctgaagatgtacaacattgccgatcggctggagacggtaatgtgggacagcaaaaagatgttccctaatctcgactggttctcggcggtctcctacaacatgatgggcgttcccaccgaaatgtttaccccgctgtttgtgattgcccgcgttacaggttgggcggcgcacatcatcgagcaacgacaggacaacaaaattatccgtccttccgccaattatattggcccggaagatcgcgcctttacgccgctggaacagcgtcagtaaacccttacctctaacgataaaaaggagttgcaccctatgtccgcacctgtttcgaacgtccgccctgaatttgaccgtgaaattgttgatattgttgattatgtgatgaagtacaacatcacctcaaaagtggcttatgacaccgcgcactactgtctgcttgataccctgggctgtgggctggaagcgctggaatatccggcctgtaaaaaattgatggggcctatcgtgccaggtaccgtggtgccgaacggtgtacgtgtaccgggcactcagttccagctcgatccggtgcaggcggcatttaatattggcgcgatgatccgctggctcgactttaacgatacctggcttgccgctgagtggggacacccttccgataacctcggcggtattctggcgaccgccgactggttgtcgcgcaacgccgtcgccgccggtaaagcgccgctgaccatgcagcaggtgctgaccgggatgatcaaagcccacgaaatccagggctgtatcgcgctggaaaactcgtttaaccgcgtgggtctcgatcacgttttgctggtgaaagtggcttccacggctgtagtggctgaaatgctcggcctgacccgcgatgaaattctcaacgccgtatcgctggcgtgggtggatgggcagtcgctgcgtacctatcgccatgcgccaaacaccggtacgcgcaaatcctgggcggcaggcgatgccacttcacgcgcggtgcgtctggcgctgatggcgaaaactggcgagatgggctatccctcggcgttgaccgccaaaacctggggcttttatgacgtctcgttcaaaggcgaaaaattccgtttccagcgcccgtacggctcctacgtgatggaaaacgtgctgttcaaaatctccttcccggcggagttccattcgcagaccgccgttgaagcagcgatgacgctgtatgagcagatgcaggcggctggaaaaacggcggcggatatcgaaaaagtaacgattcgcacccatgaagcctgtatacgcatcattgataaaaaaggcccgctgaataatccggctgaccgcgatcactgtattcagtatatggtggcgatcccactgctgttcggacgcttaacggcggcggattatgaggatggcgtggcgcaggataaacgtattgacgcgctgcgtgaaaaaacgcattgctttgaagacccggcgtttaccactgattatcatgacccggaaaaacgttcgattgccaacgccattagtcttgaatttactgacggtacccgttttgacgaggtggttgtcgagtacccgatcggccacgcgcgtcgtcgcggcgacggcattccaaaacttatcgaaaaatttaaaatcaatctggcgcgccagttcccaccccgccagcaacaacgcatcctggatgtctccctggacagaacgcgcctggagcagatgccggttaatgagtatctcgacttgtacgtcatctag

SEQ ID NO: 85 - protein, 528 aa, Artificial Sequence
  Description of Artificial Sequence Synthetic polypeptide
MAHPPRLNDDKPVIWTVSVTRLFELFRDISLEFDHLANITPIQLGFEKAVAYIRKKLASERCDAIIAAGSNGAYLKSRLSVPVILIKPSGYDVLQALAKAGKLTSSIGVVTYQETIPALVAFQKTFNLRLDQRSYITEEDARGQINELKANGTEAVVGAGLITDLAEEAGMTGIFIYSAATVRQAFSDALDMTRMSLRHNTHDATRNALRTRYVLGDMLGQSPQMEQVRQTILLYARSSAAVLIEGETGTGKELAAQAIHREYFARHDVRQGKKSHPFVAVNCGAIAESLLEAELFGYEEGAFTGSRRGGRAGLFEIAHGGTLFLDEIGEMPLPLQTRLLRVLEEKEVTRVGGHQPVPVDVRVISATHCNLEEDMQQGQFRRDLFYRLSILRLQLPPLRERVADILPLAESFLKMSLAALSVPFSAALRQGLETCQIVLLLYDWPGNIRELRNMMERLALFLSVEPTPDLTPQFLQLLLPELARESAKTPIPGLLTAQQALEKFNGDKTAAANYLGISRTTFWRRLKS

SEQ ID NO: 86 - DNA, 1587 bp, Artificial Sequence
  Description of Artificial Sequence Synthetic polynucleotide
tcagcttttcagccgccgccagaacgtcgtccggctgatacctaaataattcgccgctgctgtcttatcgccattaaatttctccagtgcctgttgtgctgtcagcaagcctggaatgggagtcttcgccgactcgcgcgccagttccggcagtagcagctgcaaaaattgcggcgttaaatccggcgtcggttccacacttaaaaataacgccagtcgttccatcatattgcgcagttcacgaatattgcccggccagtcgtagagcaataatacaatctgacaggtctctaatccctgacgtaatgcggcagaaaaagggacagagagtgccgccagagacattttcaaaaagctttccgccagcggcagaatatccgccacccgctcgcgtagcggcggcagttgcaggcgtaaaatactcagccgataaaacaggtcacggcgaaactgcccttgctgcatatcttcttccagattgcagtgagtggcgctaatgacccgcacatctaccggaacaggctgatgcccgccgacgcgggtgacctctttttcttccagcacccgtaacaggcgagtctgcaacggcagcggcatttcgccaatctcatccagaaacagcgtaccgccgtgggcaatttcgaacagcccggcgcgacctccgcgtcgcgagccggtgaacgccccttcctcatagccaaacagctctgcttccagcagcgattcggcaatcgccccgcagttgacggcaacaaacggatgtgactttttgccctgtcgcacatcgtggcgggcaaaatattctcgatgaatcgcctgggccgccagctctttgcccgtccccgtttccccctcaatcaacaccgccgcactggagcgggcatacagcaaaatagtctgccgcacctgttccatctgtggtgattgaccgagcatatcgcccagcacgtaacgagtacgcagggcgttgcgggtggcatcgtgagtgttatggcgtaacgacatgcgcgtcatatccagcgcatcgctaaatgcctggcgcacggtggcggcagaatagataaaaattccggtcattccggcttcttctgccagatcggtaatcagccccgcgccgaccaccgcttcggtgccgttggcttttagctcgttaatctgcccgcgtgcgtcttcttcggtaatgtagctacgttggtcgaggcgcaaattaaaggttttttgaaacgctaccagtgccggaatggtttcctgataggtgacaacgccgatagaagaggtgagttttccggcttttgccagcgcctgtaacacatcgtagccactcggttttatcagaatcaccggtaccgacaggcggcttttcaggtacgcaccgttagagccagcggcaatgatggcgtcgcagcgttcgctggccagttttttgcggatgtaggccaccgctttttcaaagccaagctgaataggggtgatgttcgccagatgatcaaactcgaggctgatatcgcgaaacagctcgaacagccgcgttacagataccgtccagataaccggtttgtcgtcattcagccgtggtggatgtgccat

SEQ ID NO: 87 - protein, 551 aa, Artificial Sequence
  Description of Artificial Sequence Synthetic polypeptide
MNSTILLAQDAVSEGVGNPILNISVFVVFIIVTMTVVLRVGKSTSESTDFYTGGASFSGTQNGLAIAGDYLSAASFLGIVGAISLNGYDGFLYSIGFFVAWLVALLLVAEPLRNVGRFTMADVLSFRLRQKPVRVAAACGTLAVTLFYLIAQMAGAGSLVSVLLDIHEFKWQAVVVGIVGIVMIAYVLLGGMKGTTYVQMIKAVLLVGGVAIMTVLTFVKVSGGLTTLLNDAVEKHAASDYAATKGYDPTQILEPGLQYGATLTTQLDFISLALALCLGTAGLPHVLMRFYTVPTAKEARKSVTWAIVLIGAFYLMTLVLGYGAAALVGPDRVIAAPGAANAAAPLLAFELGGSIFMALISAVAFATVLAVVAGLAITASAAVGHDIYNAVIRNGQSTEAEQVRVSRITVVVIGLISIVLGILAMTQNVAFLVALAFAVAASANLPTILYSLYWKKFNTTGAVAAIYTGLISALLLIFLSPAVSGNDSAMVPGADWAIFPLKNPGLVSIPLAFIAGWIGTLVGKPDNMDDLAAEMEVRSLTGVGVEKAVDH

SEQ ID NO: 88 - DNA, 1656 bp, Artificial Sequence
  Description of Artificial Sequence Synthetic polynucleotide
atgaattccactattctccttgcacaagacgctgtttctgagggcgtcggtaatccgattcttaacatcagtgtcttcgtcgtcttcattattgtgacgatgaccgtggtgcttcgcgtgggcaagagcaccagcgaatccaccgacttctacaccggtggtgcttccttctccggaacccagaacggtctggctatcgcaggtgactacctgtctgcagcgtccttcctcggaatcgttggtgcaatttcactcaacggttacgacggattcctttactccatcggcttcttcgtcgcatggcttgttgcactgctgctcgtggcagagccacttcgtaacgtgggccgcttcaccatggctgacgtgctgtccttccgactgcgtcagaaaccagtccgcgtcgctgcggcctgcggtaccctcgcggttaccctcttttacttgatcgctcagatggctggtgcaggttcgcttgtgtccgttctgctggacatccacgagttcaagtggcaggcagttgttgtcggtatcgttggcattgtcatgatcgcctacgttcttcttggcggtatgaagggcaccacatacgttcagatgattaaggcagttctgctggtcggtggcgttgccattatgaccgttctgaccttcgtcaaggtgtctggtggcctgaccacccttttaaatgacgctgttgagaagcacgccgcttcagattacgctgccaccaaggggtacgatccaacccagatcctggagcctggtctgcagtacggtgcaactctgaccactcagctggacttcatttccttggctctcgctctgtgtcttggaaccgctggtctgccacacgttctgatgcgcttctacaccgttcctaccgccaaggaagcacgtaagtctgtgacctgggctatcgtcctcattggtgcgttctacctgatgaccctggtccttggttacggcgctgcggcactggtcggtccagaccgcgtcattgccgcaccaggtgctgctaatgctgctgctcctctgctggccttcgagcttggtggttccatcttcatggcgctgatttccgcagttgcgttcgctaccgttctcgccgtggtcgcaggtcttgcaattaccgcatccgctgctgttggtcacgacatctacaacgctgttatccgcaacggtcagtccaccgaagcggagcaggtccgagtatcccgcatcaccgttgtcgtcattggcctgatttccattgtcctgggaattcttgcaatgacccagaacgttgcgttcctcgtggccctggccttcgcagttgcagcatccgctaacctgccaaccatcctgtactccctgtactggaagaagttcaacaccaccggcgctgtggccgctatctacaccggtctcatctccgcgctgctgctgatcttcctgtccccagcagtctccggtaatgacagcgcaatggttccaggtgcagactgggcaatcttcccactgaagaacccaggcctcgtctccatcccactggcattcatcgctggttggatcggcactttggttggcaagccagacaacatggatgatcttgctgccgaaatggaagttcgttccctcaccggtgtcggtgttgaaaaggctgttgatcactaa

SEQ ID NO: 89 - protein, 506 aa, Artificial Sequence
  Description of Artificial Sequence Synthetic polypeptide
MDLTTLITFIVYLLGMLAIGLIMYYRTNNLSDYVLGGRDLGPGVAALSAGASDMSGWLLLGLPGAIYASGMSEAWMGIGLAVGAYLNWQFVAKRLRVYTEVSNNSITIPDYFENRFKDNSHILRVISAIVILLFFTFYTSSGMVAGAKLFEASFGLQYETALWIGAVVVVSYTLLGGFLAVAWTDFIQGILMFLALIVVPIVALDQMGGWNQAVQAVGEINPSHLNMVEGVGIMAIISSLAWGLGYFGQPHIIVRFMALRSAKDVPKAKFIGTAWMILGLYGAIFTGFVGLAFISTQEVPILSEFGIQVVNENGLQMLADPEKIFIAFSQILFHPVVAGILLAAILSAIMSTVDSQLLVSSSAVAEDFYKAIFRKKATGKELVWVGRIATVIIAIVALIIAMNPDSSVLDLVSYAWAGFGAAFGPIIILSLFWKRITRNGALAGIIVGAITVIVWGDFLSGGIFDLYEIVPGFILNMIVTVIVSLIDKPNPDLEADFDETVEKMKE

SEQ ID NO: 90 - DNA, 1521 bp, Artificial Sequence
  Description of Artificial Sequence Synthetic polynucleotide
atggatcttacgacattaataacttttatagtatatctactagggatgttggcgattggcctcatcatgtattatcgaaccaataatttatcagattatgttcttggtggacgtgatcttggtccaggcgtagctgcattgagtgctggtgcatcggatatgagtggttggctgttattaggtttgcctggagcgatttatgcatctggtatgtctgaagcttggatggggatcgggttagctgtaggtgcttatttaaattggcaatttgtagctaagcgattacgcgtttataccgaggtatcaaataattccattacgatcccagattattttgaaaatcggtttaaagataactcacatattcttcgtgttatatctgctatcgtaattttgttattcttcactttttatacatcttcaggaatggttgcaggagcaaaattatttgaggcttcattcggtctccaatacgaaactgctctgtggattggtgcggttgtagttgtatcttatacgttacttggaggatttctagcggttgcatggacagactttattcaaggtattcttatgttccttgcactaattgttgttccaatcgtcgcattagatcaaatgggtggctggaatcaagcggtacaagctgttggtgaaattaatccttcccacctcaatatggttgaaggtgttggaataatggcaattatttcatcacttgcttggggcttaggttattttggacagccacatattattgttcgttttatggcattacgttcggcgaaagatgttccgaaagcgaaatttattggaacagcttggatgattttaggactttatggagcaatctttactggttttgtaggactagcatttatcagtacacaagaagtaccgattctgtctgaattcgggattcaagtagttaatgagaatggtttacaaatgttagccgatcctgaaaagatatttattgctttctcccaaatactattccatccagtagttgccggtatcttactagcggcaatcttgtctgcaattatgagtaccgttgattcacagttacttgtatcatcttcagcggttgcagaagatttctataaagctattttccgtaaaaaagctactggtaaagagcttgtttgggttggacgtattgctacagtgataattgcgattgttgctttaattattgcaatgaacccagatagctctgtattggatctagttagttatgcatgggctggatttggtgcagcatttggaccaattatcatcttgtcattattctggaagagaatcacaagaaatggtgcactagcgggtatcattgtaggtgccattacggtaattgtatggggagactttctatctggaggtatctttgacctctacgaaattgttccaggctttatcttaaatatgattgtcaccgttattgtgagtcttatcgataaaccgaatccagatttagaagctgactttgatgaaaccgtagaaaaaatgaaagaataa

SEQ ID NO: 91 - protein, 403 aa, Artificial Sequence
  Description of Artificial Sequence Synthetic polypeptide
MSTRTPSSSSSRLMLTIGLCFLVALMEGLDLQAAGIAAGGIAQAFALDKMQMGWIFSAGILGLLPGALVGGMLADRYGRKRILIGSVALFGLFSLATAIAWDFPSLVFARLMTGVGLGAALPNLIALTSEAAGPRFRGTAVSLMYCGVPIGAALAATLGFAGANLAWQTVFWVGGVVPLILVPLLMRWLPESAVFAGEKQSAPPLRALFAPETATATLLLWLCYFFTLLVVYMLINWLPLLLVEQGFQPSQAAGVMFALQMGAASGTLMLGALMDKLRPVTMSLLIYSGMLASLLALGTVSSFNGMLLAGFVAGLFATGGQSVLYALAPLFYSSQIRATGVGTAVAVGRLGAMSGPLLAGKMLALGTGTVGVMAASAPGILVAGLAVFILMSRRSRIQPCADA

SEQ ID NO: 92 - DNA, 1212 bp, Artificial Sequence
  Description of Artificial Sequence Synthetic polynucleotide
atgtcgactcgtaccccttcatcatcttcatcccgcctgatgctgaccatcgggctttgttttttggtcgctctgatggaagggctggatcttcaggcggctggcattgcggcgggtggcatcgcccaggctttcgcactcgataaaatgcaaatgggctggatatttagcgccggaatactcggtttgctacccggcgcgttggttggcggaatgctggcggaccgttatggtcgcaagcgcattttgattggctcagttgcgctgtttggtttgttctcactggcaacggcgattgcctgggatttcccctcactggtctttgcgcggctgatgaccggtgtcgggctgggggcggcgttgccgaatcttatcgccctgacgtctgaagccgcgggtccacgttttcgtgggacggcagtgagcctgatgtattgcggtgttcccattggcgcggcgctggcggcgacactgggtttcgcgggggcaaacttagcatggcaaacggtgttttgggtaggtggtgtggtgccgttgattctggtgccgctattaatgcgctggctgccggagtcggcggttttcgctggcgaaaaacagtctgcgccaccactgcgtgccttatttgcgccagaaacggcaaccgcgacgctgctgctgtggttgtgttatttcttcactctgctggtggtctacatgttgatcaactggctaccgctacttttggtggagcaaggattccagccatcgcaggcggcaggggtgatgtttgccctgcaaatgggggcggcaagcgggacgttaatgttgggcgcattgatggataagctgcgtccagtaaccatgtcgctactgatttatagcggcatgttagcttcgctgctggcgcttggaacggtgtcgtcatttaacggtatgttgctggcgggatttgtcgcggggttgtttgcgacaggtgggcaaagcgttttgtatgccctggcaccgttgttttacagttcgcagatccgcgcaacaggtgtgggaacagccgtggcggtagggcgtctgggggctatgagcggtccgttactggccgggaaaatgctggcattaggcactggcacggtcggcgtaatggccgcttctgcaccgggtattcttgttgctgggttggcggtgtttattttgatgagccggagatcacgaatacagccgtgcgccgatgcctga

SEQ ID NO: 93 - DNA, 5631 bp, Artificial Sequence; identical to SEQ ID NO: 55
  Description of Artificial Sequence Synthetic polynucleotide

SEQ ID NO: 94 - DNA, 5556 bp, Artificial Sequence
  Description of Artificial Sequence Synthetic polynucleotide
atgacgttacactcaccgggtcaggcgtttcgcgctgcgcttgctaaagaaaaaccattacaaattgtcggcgctatcaacgccaatcatgctctgttagcccagagggctgggtatcaggctctctatctctcgggcggcggtgttgccgcaggctcgctggggctaccggatctgggcatctccacccttgatgacgtattgaccgatatccgccgtatcaccgacgtctgcccgctgccgctgctggtggatgccgatattggcttcggatcgtcggcgtttaacgtagcgcgtaccgtgaaatcgatttccaaagccggcgccgccgcgctgcatattgaagatcagattggcgccaagcgctgcgggcatcggccaaataaagcgatcgtctcgaaagaagagatggtggaccggatccacgcggcggtggatgcgcggaccgatcctgactttgtcattatggcgcgtaccgatgcgctggcggttgaaggccttgatgccgctatcgatcgcgcgcgggcctacgtagaggccggtgccgacatgctgttcccggaggcgattactgaacttgcgatgtaccgccagtttgccgacgcagtgcaggtgccaatccttgccaatattaccgaattcggcgcgacgccgttgtttactaccgaagagctacgcaacgccaacgtggcgatggcgctctatccgctgtcggcgttccgggcgatgaatcgcgcggcggagaaggtttacaacgtgctgcgacaggaaggaacgcaaaagagcgttatcgacatcatgcagacccgtaatgagctgtatgaaagcatcaattattaccagttcgaggaaaaacttgacgcgctgtacgccaaaaaatcgtaggccacgggtctgataaagcgtagccgctatcaagtctgtggcggacaacctcaataccctacacattacaaaaatgacgaggacactatgagcgacacgacgatcctgcaaaacaacacaaatgtcattaagccaaaaaaatccgtcgcattatccggcgtacccgccggaaataccgccttatgcaccgtaggtaaaagcggtaacgatctgcactatcgcgggtacgatattctcgatctcgcggagcactgtgaatttgaagaagttgcgcatctgctcattcacggcaagctgcccacccgtgatgagctgaatgcctataaaagcaaattaaaagcgctgcgtggcttacccgctaacgtccgtaccgtgctggaagcgctgccagcggcatcgcacccgatggacgtaatgcgcaccggcgtttctgcgctgggctgcaccctgccggaaaaagaggggcataccgtttctggcgcgcgtgatatcgccgacaagctgctggcctccctcagctccattctcctttactggtatcactacagccacaacggcgaacgcattcagccagaaactgacgatgactctatcggcgggcatttcctgcatttattacacggcgaaaagccatcgcaaagctgggaaaaggcgatgcacatttcactggtactgtacgccgaacatgagttcaacgcctcaacctttaccagccgggtggtagccggtacgggatcggatatgtactccgccatcattggcgcgataggcgcgcttcgcgggccgaagcacggcggggcgaatgaagtctcgctggagattcagcagcgctacgaaacgccggatgaagcagaagccgatatccgtaaacgtatcgccaataaagaagtggtgattggttttggtcatccggtatacaccatcgccgatccgcgccatcaggtgattaagcgggtagcgaagcagctttcacaggagggcggttcgctgaagatgtacaacattgccgatcggctggagacggtaatgtgggacagcaaaaagatgttccctaatctcgactggttctcggcggtctcctacaacatgatgggcgttcccaccgaaatgtttaccccgctgtttgtgattgcccgcgttacaggttgggcggcgcacatcatcgagcaacgacaggacaacaaaattatccgtccttccgccaattatattggcccggaagatcgcgcctttacgccgctggaacagcgtcagtaaacccttacctctaacgataaaaaggagttgcaccctatgtccgcacctgtttcgaacgtccgccctgaatttgaccgtgaaattgttgatattgttgattatgtgatgaagtacaacatcacctcaaaagtggcttatgacaccgcgcactactgtctgcttgataccctgggctgtgggctggaagcgctggaatatccggcctgtaaaaaattgatggggcctatcgtgccaggtaccgtggtgccgaacggtgtacgtgtaccgggcactcagttccagctcgatccggtgcaggcggcatttaatattggcgcgatgatccgctggctcgactttaacgatacctggcttgccgctgagtggggacacccttccgataacctcggcggtattctggcgaccgccgactggttgtcgcgcaacgccgtcgccgccggtaaagcgccgctgaccatgcagcaggtgctgaccgggatgatcaaagcccacgaaatccagggctgtatcgcgctggaaaactcgtttaaccgcgtgggtctcgatcacgttttgctggtgaaagtggcttccacggctgtagtggctgaaatgctcggcctgacccgcgatgaaattctcaacgccgtatcgctggcgtgggtggatgggcagtcgctgcgtacctatcgccatgcgccaaacaccggtacgcgcaaatcctgggcggcaggcgatgccacttcacgcgcggtgcgtctggcgctgatggcgaaaactggcgagatgggctatccctcggcgttgaccgccaaaacctggggcttttatgacgtctcgttcaaaggcgaaaaattccgtttccagcgcccgtacggctcctacgtgatggaaaacgtgctgttcaaaatctccttcccggcggagttccattcgcagaccgccgttgaagcagcgatgacgctgtatgagcagatgcaggcggctggaaaaacggcggcggatatcgaaaaagtaacgattcgcacccatgaagcctgtatacgcatcattgataaaaaaggcccgctgaataatccggctgaccgcgatcactgtattcagtatatggtggcgatcccactgctgttcggacgcttaacggcggcggattatgaggatggcgtggcgcaggataaacgtattgacgcgctgcgtgaaaaaacgcattgctttgaagacccggcgtttaccactgattatcatgacccggaaaaacgttcgattgccaacgccattagtcttgaatttactgacggtacccgttttgacgaggtggttgtcgagtacccgatcggccacgcgcgtcgtcgcggcgacggcattccaaaacttatcgaaaaatttaaaatcaatctggcgcgccagttcccaccccgccagcaacaacgcatcctggatgtctccctggacagaacgcgcctggagcagatgccggttaatgagtatctcgacttgtacgtcatctagaacctgtctcattaggcgtaagttctacaggagagcattatgtcttttagcgaattttatcagcgttcgattaacgaaccggagcagttctgggctgaacaggcccggcgtatcgactggcagcagccgtttacgcagacgctggactacagcaacccgccgtttgcccgctggttttgcggcggcaccactaatctgtgccataacgcgattgaccgctggctggatacccagccggatgcgctggcgctgattgcggtttcctctgagaccgaagaagaacgtaccttcacctttcgtcaactgtatgacgaggtgaatgtcgtggcctctatgctgctgtcactgggcgtgcggcgtggcgatcgggtactggtgtatatgccgatgattgccgaggcgcacatcacattactggcctgcgcgcgcattggcgcgatccattcagtggtgtttggtggttttgcctcgcacagtgtagccgcgcgcatcgacgatgccagaccggtgctgattgtctcggcggacgccggagcgcgaggtgggaaggtcattccctataaaaagcttcttgatgaggcggtcgatcaggcacagcatcagccgaagcatgtactgctggtggatcgggggctggcgaaaatggcgcgggttgccgggcgcgatgtggattttgcgaccctgcgcgaacaccatgccggggcgcgtgtgccagtggcctggcttgaatctaatgaaagttcctgcattctttatacctccggcactaccggcaaaccgaaaggcgttcagcgtgacgttggtggctacgccgtggcgctggcgacatcgatggacaccctctttggcggcaaagcgggcggcgtctttttctgcgcttcggatatcggttgggtagtggggcactcttatattgtgtatgcgccgctgctggcgggtatggcgaccatcgtttatgaaggattgccgacgtatccggactgcggcgtatggtggaaaattgtcgagaaatatcgggtgagccggatgttttcagcgccaaccgccattcgtgtgctgaagaaatttcccaccgcgcagatacgcaatcatgatctctcctcgctggaagttctctatctggcaggcgagccgctcgacgagccaacggcagcctgggttagcggaacactgggtgtgccggtgatcgacaattactggcagaccgaatccggctggccgattatggcgctggcgcgcacgcttgatgacagaccatcgcgtttgggcagtcccggcgtgccgatgtacggctataatgttcaactgctcaacgaggtgaccggtgaaccctgtggtgcgaacgaaaagggaatggtggttattgaagggccgctgccgccgggctgcattcagaccatctggggcgatgacgcacgctttgtgaatacctactggtcactgtttactcgtcaggtgtatgccacctttgactgggggatccgcgacgccgacggctattattttatccttgggcgcacggatgatgtgatcaacgtcgccggacatcgtctcggcacccgtgagatagaggagagcatctccagctatcccaacgttgcggaagtggcggtggtaggggtaaaagacgcgctgaaagggcaggtagcggtagccttcgtgatcccgaaacagagtgacagtctggaagaccgcgaagtggcgcattcggaagagaaggcgattatggcgctggtcgatagtcagatcggcaactttggccgcccggcgcacgtgtggtttgtctcgcagctaccaaaaacccgatccgggaagatgctcagacgaacgatccaggcgatctgcgagggccgggatccaggcgatctgacgaccattgacgatccgacgtcgttgcaacaaattcgccaggtcattgaggagtaa

SEQ ID NO: 95 - protein, 540 aa, Artificial Sequence
  Description of Artificial Sequence Synthetic polypeptide
MTDIMDSQAVKAAAAASAANAAQPSAHQPLRTAVVKAAELARAAEERARDKQHAKGKKTARERLDLLFDTGTFEEIGRFQGGNIAGGNAGAAVITGFGQVYGRKVAVYAQDFSVKGGTLGTAEGEKICRLMDMAIDLKVPIVAIVDSGGARIQEGVAALTQYGRIFRKTCEASGFVPQLSLILGPCAGGAVYCPALTDLIIMTRENSNMFVTGPDVVKASTGETISMADLGGGEVHNRVSGVAHYLGEDESDAIDYARTVLAYLPSNSESKPPVYAYAVTRAERETAKRLATIVPTNERQPYDMLEVIRCIVDYGEFVQVQELFGASALVGFACIDGKPVGIVANQPNVLAGILDVDSSEKVARFVRLCDAFNLPVVTLVDVPGYKPGSDQEHAGIIRRGAKVIYAYANAQVPMVTVVLRKAFGGAYIVMGSKAIGADLNFAWPSSQIAVLGAAGAVNIIHRHDLAKAKASGQDVDALRAKYIKEYETSTVNANLSLEIGQIDGMIDPEQTREVIVESLATLATKRRVKRTTKHHGNQPL

SEQ ID NO: 96 - DNA, 1623 bp, Artificial Sequence
  Description of Artificial Sequence Synthetic polynucleotide
tcagaggggctggttgccgtggtgtttggtggtgcgcttgacgcgccgcttggtggcgagcgtggccagcgattcgacaatcacctcacgggtctgttcggggtcgatcatgccgtcgatctgcccgatttccagtgacaggttcgcgttgacggtgctggtctcgtactccttgatgtacttggcccgcagcgcatcgacgtcctgtccggaggccttggccttggccaggtcgtggcggtggatgatgttcaccgcgccggccgcgccgagcaccgcgatctgggaggagggccacgcgaagttcaggtccgcgccaatggccttggatcccatcacgatgtacgcgccgccgaacgccttgcgcaacaccacggtcaccatcggtacctgtgcgttggcgtaggcgtagatcaccttggcgccgcggcggatgatgccggcgtgttcctggtcggagccgggcttgtagccgggcacatccacgagggtgaccacgggcaggttgaacgcgtcgcacaggcgtacgaatcgggcgactttctcggacgagtcgacgtccaggatgccggcgagcacgttcggctggttcgccacgatgccaaccggcttgccgtcgatgcaggcgaagccgacgagcgcggaggcgccgaacagttcctgcacctgcacgaattcgccgtaatcgacgatgcaacgaatcacttcgagcatgtcgtaaggctgacgttcgttggtgggcacgatggtggcaagtcgcttggcggtctcgcgttcggcgcgggtgacggcgtatgcgtagaccggcggcttgctttcgctgttggacggcaggtaggcgagcacggtgcgcgcatagtcgatggcgtcggattcgtcctcgccgaggtagtgggccacgccggacacccggttgtgcacttcgccgccgccgaggtcggccatggagatggtctcgccggtcgaggccttgaccacgtccggtccggtgacgaacatgttcgagttctcacgggtcatgatgatgaggtccgtcagggccgggcagtagacggcaccgccggcgcaggggccgagaatcaggctcagctggggcacgaagccgctggcctcgcaagtcttgcggaagatgcgaccgtactgggtcagggcggccacgccctcctggatgcgggcgccgccggagtccacgatggccacgatcggcactttgaggtcgatggccatgtccatcagtcggcagatcttctcgccttcggcggtgccgagggtgccgcccttgacggagaagtcctgggcgtagacggccactttgcggccgtagacctggccgaagccggtgatgacggccgcaccggcgttgccgccggcgatattgccgccctggaagcggccgatctcctcgaacgtgccggtgtcgaagagcaggtcgaggcgttcgcgcgcggttttcttgcctttggcgtgctgcttgtcgcgggcgcgctcttcggcggcgcgggccagttcggcggccttgaccacagcggtgcgcagcggctggtgggccgaaggctgggcggcgttggcggccgaggccgcagccgcggccttcacggcctgcgaatccatgatgtcagtcat

SEQ ID NO: 97 - protein, 427 aa, Artificial Sequence
  Description of Artificial Sequence Synthetic polypeptide
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 98 - DNA, 1284 bp, Artificial Sequence
  Description of Artificial Sequence Synthetic polynucleotide
atggctgatacaaaagcaaaactcaccctcaacggggatacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccatgctttccgtcgcgactcgcatccaatggcagtcatgtgtggtattaccggcgcgctggcggcgttctatcacgactcgctggatgttaacaatcctcgtcaccgtgaaattgccgcgttccgcctgctgtcgaaaatgccgaccatggccgcgatgtgttacaagtattccattggtcagccatttgtttacccgcgcaacgatctctcctacgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggcggtgctaacgaagcggcgctgaaaatgctggaagaaatcagctccgttaaacacattccggaatttgttcgtcgtgcgaaagacaaaaatgattctttccgcctgatgggcttcggtcaccgcgtgtacaaaaattacgacccgcgcgccaccgtaatgcgtgaaacctgccatgaagtgctgaaagagctgggcacgaaggatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 99 - protein, 392 aa, Artificial Sequence
  Description of Artificial Sequence Synthetic polypeptide
MKDVVIVAAKRTAIGSFLGSLASLSAPQLGQTAIRAVLDSANVKPEQVDQVIMGNVLTTGVGQNPARQAAIAAGIPVQVPASTLNVVCGSGLRAVHLAAQAIQCDEADIVVAGGQESMSQSAHYMQLRNGQKMGNAQLVDSMVADGLTDAYNQYQMGITAENIVEKLGLNREEQDQLALTSQQRAAAAQAAGKFKDEIAVVSIPQRKGEPVVFAEDEYIKANTSLESLTKLRPAFKKDGSVTAGNASGINDGAAAVLMMSADKAAELGLKPLARIKGYAMSGIEPEIMGLGPVDAVKKTLNKAGWSLDQVDLIEANEAFAAQALGVAKELGLDLDKVNVNGGAIALGHPIGASGCRILVTLLHEMQRRDAKKGIATLCVGGGMGVALAVERD

SEQ ID NO: 100 - protein, 248 aa, Artificial Sequence
  Description of Artificial Sequence Synthetic polypeptide
MSEQKVALVTGALGGIGSEICRQLVTAGYKIIATVVPREEDREKQWLQSEGFQDSDVRFVLTDLNNHEAATAAIQEAIAAEGRVDVLVNNAGITRDATFKKMSYEQWSQVIDTNLKTLFTVTQPVFNKMLEQKSGRIVNISSVNGLKGQFGQANYSASKAGIIGFTKALAQEGARSNICVNVVAPGYTATPMVTAMREDVIKSIEAQIPLQRLAAPAEIAAAVMYLVSEHGAYVTGETLSINGGLYMH

SEQ ID NO: 101 - protein, 590 aa, Artificial Sequence
  Description of Artificial Sequence Synthetic polypeptide
MNPNSFQFKENILQFFSVHDDIWKKLQEFYYGQSPINEALAQLNKEDMSLFFEALSKNPARMMEMQWSWWQGQIQIYQNVLMRSVAKDVAPFIQPESGDRRFNSPLWQEHPNFDLLSQSYLLFSQLVQNMVDVVEGVPDKVRYRIHFFTRQMINALSPSNFLWTNPEVIQQTVAEQGENLVRGMQVFHDDVMNSGKYLSIRMVNSDSFSLGKDLAYTPGAVVFENDIFQLLQYEATTENVYQTPILVVPPFINKYYVLDLREQNSLVNWLRQQGHTVFLMSWRNPNAEQKELTFADLITQGSVEALRVIEEITGEKEANCIGYCIGGTLLAATQAYYVAKRLKNHVKSATYMATIIDFENPGSLGVFINEPVVSGLENLNNQLGYFDGRQLAVTFSLLRENTLYWNYYIDNYLKGKEPSDFDILYWNSDGTNIPAKIHNFLLRNLYLNNELISPNAVKVNGVGLNLSRVKTPSFFIATQEDHIALWDTCFRGADYLGGESTLVLGESGHVAGIVNPPSRNKYGCYTNAAKFENTKQWLDGAEYHPESWWLRWQAWVTPYTGEQVPARNLGNAQYPSIEAAPGRYVLVNLF

SEQ ID NO: 102 - DNA, 5617 bp, Artificial Sequence
  Description of Artificial Sequence Synthetic polynucleotide
aagcttatagctaacaccgcaatcaattttttcactcgtctagcgtctgtcaagcgcgtattttcaagattaaacccgcgtcctttgagacaactgaataaggtttcaatttcccagcgtaatgcataatcctgaatagcattggcattaaactgaggagaaacgacgagtaaaagctctccattttctaactgtagtgcacttatatatagtttcacccgaccaaccaaaatccgtcgtttacgacattcaatttgaccaactttaagatggcgaaataaatcactaattttatgattctttcctaaatgattggtgacaatgaagtttttttaacacgaatgcagaagttgatgtcttggttcaattaaccatgtaaaccactgctcaccgataaactctctgtctgcgaacacattcacaatacggtctttaccaaaaatggctataaagcgttgaatcaaagcaatacgctctttcgtatctgaatttccacgtttattaagcaatgtccaaaggataggtatcgctattccacgataaacgattgcgagcatcaggatattaatatttcgttttccccatttccaattggttctatctaaagtcagttgcacttggtcgaatgaaaacatattgaaaatcaactgagaaatttgacgataatcaaaatactgacctgcaaagaagcgctgcatacgtcgataaaatgattgtggtaagcacttgatgggcaaggctttagatgcagaagaaagattacatgtttgctttaaaataatcacaagcatgatgagcgcaaagcactttaaatgtgacttgttccattttagatatttgtttaagataagatataactcattgagatgtgtcatagtattcgtcgttagaaaacaattattatgacattatttcaatgagttatctatttttgtcgtgtacagagcaatatttgtttacttttgactttaaagcatcatcaaactgcgatctgtttgcaatataaaacgcttaatttctaaacaagaataaagaggaaaaacttcttatttttttataaccttattctgcttaggaaaacaacatgtctgaacaaaaagtagctttagtgacgggtgcattaggtggtattggcagtgagatttgccgtcaactggttacagcaggctataaaattatcgcaacggttgtaccacgtgaagaagaccgcgaaaaacaatggcttcaaagtgaaggatttcaagacagcgatgtacgctttgtcttaacggatttaaataaccacgaagcggcaacagcagctattcaagaagcaattgctgctgaaggtcgtgtcgatgtgctggtcaataatgcaggcatcacccgtgatgcaacttttaaaaagatgagctatgaacagtggtcacaagtcattgataccaacttaaaaacattgttcacagtgactcagcccgtgtttaacaaaatgttagaacaaaagtcgggacgtattgtcaatatcagctcagtcaacggtttaaaaggtcagtttggacaagccaactactctgcgagcaaagccggcattatcggttttaccaaagccttagctcaagaaggtgcacgttcaaatatttgtgtaaacgtggttgcgcctggctataccgcaacaccaatggttactgccatgcgtgaagatgtgattaaaagcattgaagcacaaattcctctacaacgtcttgctgcgccagctgaaattgccgctgctgttatgtacttggtcagcgagcacggtgcgtacgtgacaggcgaaaccttatcgattaatggcggtttatacatgcactaaaccgtgcagcccctattttcatttacaagtttatttactggagttacaccatgctatacggcgacttattttcaaatatgaatgcacaatacaaaaacgtatttgaaccgtacacaaaattcaacagcttagtggctaaaaactttgctgacttaaccaacctacaattagaagcagcacgcaactatgccaacattggtctagcgcaaatgtttgccaatagtgaagttaaagacatgcaaagcatggtgaattgcaccaccaagcaattagaaaccatgaacaaacttagtcagcaaatgattgaagatggcaaaaagttggcaacactaacgactgaattcaaatcggaatttgaaaagttagttagcgaatctatgcctaacaataaataacactgctctgaaaaccatgcgttatcaggacgaatgttacggggaagtgtgaaaatttccccgttttagtttcagccctgcactcaatttgattgctaaaagccatgtgctatggagcgatgaaatgaacccgaactcatttcaattcaaagaaaacatactacaatttttttctgtacatgatgacatctggaaaaaattacaagaattttattatgggcaaagcccaattaatgaggctttggcgcagctcaacaaagaagatatgtctttgttctttgaagcactatctaaaaacccagctcgcatgatggaaatgcaatggagctggtggcaaggtcaaatacaaatctaccaaaatgtgttgatgcgcagcgtggccaaagatgtagcaccatttattcagcctgaaagtggtgatcgtcgttttaacagcccattatggcaagaacacccaaattttgacttgttgtcacagtcttatttactgtttagccagttagtgcaaaacatggtagatgtggtcgaaggtgttccagacaaagttcgctatcgtattcacttctttacccgccaaatgatcaatgcgttatctccaagtaactttctgtggactaacccagaagtgattcagcaaactgtagctgaacaaggtgaaaacttagtccgtggcatgcaagttttccatgatgatgtcatgaatagcggcaagtatttatctattcgcatggtgaatagcgactctttcagcttgggcaaagatttagcttacacccctggtgcagtcgtctttgaaaatgacattttccaattattgcaatatgaagcaactactgaaaatgtgtatcaaacccctattctagtcgtaccaccgtttatcaataaatattatgtgctggatttacgcgaacaaaactctttagtgaactggttgcgccagcaaggtcatacagtctttttaatgtcatggcgtaacccaaatgccgaacagaaagaattgacttttgccgatctcattacacaaggttcagtggaagctttgcgtgtaattgaagaaattaccggtgaaaaagaggccaactgcattggctactgtattggtggtacgttacttgctgcgactcaagcctattacgtggcaaaacgcctgaaaaatcacgtaaagtctgcgacctatatggccaccattatcgactttgaaaacccaggcagcttaggtgtatttattaatgaacctgtagtgagcggtttagaaaacctgaacaatcaattgggttatttcgatggtcgtcagttggcagttaccttcagtttactgcgtgaaaatacgctgtactggaattactacatcgacaactacttaaaaggtaaagaaccttctgattttgatattttatattggaacagcgatggtacgaatatccctgccaaaattcataatttcttattgcgcaatttgtatttgaacaatgaattgatttcaccaaatgccgttaaggttaacggtgtgggcttgaatctatctcgtgtaaaaacaccaagcttctttattgcgacgcaggaagaccatatcgcactttgggatacttgtttccgtggcgcagattacttgggtggtgaatcaaccttggttttaggtgaatctggacacgtagcaggtattgtcaatcctccaagccgtaataaatacggttgctacaccaatgctgccaagtttgaaaataccaaacaatggctagatggcgcagaatatcaccctgaatcttggtggttgcgctggcaggcatgggtcacaccgtacactggtgaacaagtccctgcccgcaacttgggtaatgcgcagtatccaagcattgaagcggcaccgggtcgctatgttttggtaaatttattctaatcggtcatataacaacagccatgcagatgctatatatcatgtgcatccacagaaacatgaacacaaaatttaaggatataaaatgaaagatgttgtgattgttgcagcaaaacgtactgcgattggtagctttttaggtagtcttgcatctttatctgcaccacagttggggcaaacagcaattcgtgcagttttagacagcgctaatgtaaaacctgaacaagttgatcaggtgattatgggcaacgtactcacgacaggcgtgggacaaaaccctgcacgtcaggcagcaattgctgctggtattccagtacaagtgcctgcatctacgctgaatgtcgtctgtggttcaggtttgcgtgcggtacatttggcagcacaagccattcaatgcgatgaagccgacattgtggtcgcaggtggtcaagaatctatgtcacaaagtgcgcactatatgcagctgcgtaatgggcaaaaaatgggtaatgcacaattggtggatagcatggtggctgatggtttaaccgatgcctataaccagtatcaaatgggtattaccgcagaaaatattgtagaaaaactgggtttaaaccgtgaagaacaagatcaacttgcattgacttcacaacaacgtgctgcggcagctcaggcagctggcaagtttaaagatgaaattgccgtagtcagcattccacaacgtaaaggtgagcctgttgtatttgctgaagatgaatacattaaagccaataccagccttgaaagcctcacaaaactacgcccagcctttaaaaaagatggtagcgtaaccgcaggtaatgcttcaggcattaatgatggtgcagcagcagtactgatgatgagtgcggacaaagcagcagaattaggtcttaagccattggcacgtattaaaggctatgccatgtctggtattgagcctgaaattatggggcttggtcctgtcgatgcagtaaagaaaaccctcaacaaagcaggctggagcttagatcaggttgatttgattgaagccaatgaagcatttgctgcacaggctttgggtgttgctaaagaattaggcttagacctggataaagtcaacgtcaatggcggtgcaattgcattgggtcacccaattggggcttcaggttgccgtattttggtgactttattacatgaaatgcagcgccgtgatgccaagaaaggcattgcaaccctctgtgttggcggtggtatgggtgttgcacttgcagttgaacgtgactaagtacaccattgcatcgaatcttgaaacttgataaagattgacaataaattcaatacataatgggagctcaggcttccattatttctagctgagcgcatttctaatattaaggcttctagctcagcattgattttagtatttggcgattttaagggacgtctactctgactacttaatccatcaataccttgctcagaatatcgtttccaccacttgcgtaacgttggtctaga

SEQ ID NO: 103 - protein, 319 aa, Artificial Sequence
  Description of Artificial Sequence Synthetic polypeptide
MSLNFLDFEQPIAELEAKIDSLTAVSRQDEKLDINIDEEVHRLREKSVELTRKIFADLGAWQIAQLARHPQRPYTLDYVRLAFDEFDELAGDRAYADDKAIVGGIARLDGRPVMIIGHQKGRETKEKIRRNFGMPAPEGYRKALRLMQMAERFKMPIITFIDTPGAYPGVGAEERGQSEAIARNLREMSRLGVPVVCTVIGEGGSGGALAIGVGDKVNMLQYSTYSVISPEGCASILWKSADKAPLAAEAMGIIAPRLKELKLIDSIIPEPLGGAHRNPEAMAASLKAQLLADLADLDVLSTEDLKNRRYQRLMSYGYA

SEQ ID NO: 104 - DNA, 960 bp, Artificial Sequence
  Description of Artificial Sequence Synthetic polynucleotide
atgagtctgaatttccttgattttgaacagccgattgcagagctggaagcgaaaatcgattctctgactgcggttagccgtcaggatgagaaactggatattaacatcgatgaagaagtgcatcgtctgcgtgaaaaaagcgtagaactgacacgtaaaatcttcgccgatctcggtgcatggcagattgcgcaactggcacgccatccacagcgtccttataccctggattacgttcgcctggcatttgatgaatttgacgaactggctggcgaccgcgcgtatgcagacgataaagctatcgtcggtggtatcgcccgtctcgatggtcgtccggtgatgatcattggtcatcaaaaaggtcgtgaaaccaaagaaaaaattcgccgtaactttggtatgccagcgccagaaggttaccgcaaagcactgcgtctgatgcaaatggctgaacgctttaagatgcctatcatcacctttatcgacaccccgggggcttatcctggcgtgggcgcagaagagcgtggtcagtctgaagccattgcacgcaacctgcgtgaaatgtctcgcctcggcgtaccggtagtttgtacggttatcggtgaaggtggttctggcggtgcgctggcgattggcgtgggcgataaagtgaatatgctgcaatacagcacctattccgttatctcgccggaaggttgtgcgtccattctgtggaagagcgccgacaaagcgccgctggcggctgaagcgatgggtatcattgctccgcgtctgaaagaactgaaactgatcgactccatcatcccggaaccactgggtggtgctcaccgtaacccggaagcgatggcggcatcgttgaaagcgcaactgctggcggatctggccgatctcgacgtgttaagcactgaagatttaaaaaatcgtcgttatcagcgcctgatgagctacggttacgcgtaa

SEQ ID NO: 105 - protein, 557 aa, Artificial Sequence
  Description of Artificial Sequence Synthetic polypeptide
MFKQDQLDKIAAKKESWSAKLAAAVKKRPEREAQFMTDSGIEVNTVYTPLDIADMDYERDLGLPGEYPYTRGVQPNMYRGRLWTMRQYAGFGTAEETNQRFRYLLEQGQTGLSCAFDLPTQIGYDSDHPMARGEIGKVGVAIDSLQDMETLFDQIPLGKVSTSMTINAPAGILLAMYIVVAEKQGFKRAELNGTIQNDIIKEYVGRGTYILPPEPSMRLITNIFEFCSKEVPNWNTISISGYHIREAGCTAAQEIAFTLADGIAYVDAAIKAGLDVDQFGPRLSFFFNAHLNFLEEIAKFRAARRVWAKIMKERFGAKDPRSWTLRFHTQTAGCSLTAQQPMVNIMRTAFEALAAVLGGTQSLHTNSYDEALALPSDESVLIALRTQQVIGYEIGVCDVVDPLGGSYYIESLTNQLEAKAWEYIEKIDALGGAVKAIDYMQKEIHNAAYQYQLAIDNKKKTVIGVNKFQLKEEEKPKNLLKVDLSVGERQIAKLKKLKEERDNAKVEALLKQVREAAQSDANMMPVFIDAVKEYVTLGEICGVLRDVFGEYKQQIVF

SEQ ID NO: 106 - DNA, 1674 bp, Artificial Sequence
  Description of Artificial Sequence Synthetic polynucleotide
ttgtttaaacaggatcaactggacaaaattgctgccaagaaagaaagctggtctgcaaagctggcagcagcggtcaaaaagcgtccggaaagagaagctcaattcatgaccgactctggaattgaagtcaacaccgtttacactcctcttgatattgcagacatggattatgagcgtgacctgggcctgcctggggaatacccgtatacccggggtgtgcagcctaacatgtaccgcggccgcctctggaccatgcgccagtacgcaggttttggcacagccgaagaaaccaaccagcgtttccgctatctcctggagcaagggcagacaggccttagctgcgccttcgatttgcctactcagatcggctacgattcggaccatcctatggcaaggggagaaatcggtaaggttggcgttgctatagactccctgcaggacatggaaactcttttcgaccagatccccctgggcaaggtcagcacttccatgaccatcaacgccccggcaggcatactactggccatgtatattgtggtggctgaaaaacaggggtttaagagggcagaattaaacggaacgattcaaaacgatattattaaggaatatgtcggccggggaacatacatcctgccgcctgagccctcaatgcgtttaattacaaatatttttgagttctgttccaaagaagtgcccaactggaatacgatcagcatcagcggctatcatatccgtgaagcgggttgcaccgcagctcaggaaatagcctttaccctagcggacggcattgcctatgtggatgcagccattaaagcaggcctggatgttgatcagtttggtcctcgcctttcattcttcttcaatgctcacctgaacttcctcgaggaaattgcaaaattccgggcggcacggcgcgtctgggcgaagattatgaaggaacgtttcggagccaaagatccgcgctcgtggaccctgcgcttccacactcagactgccggctgcagcctgacggcccagcagccgatggtaaatatcatgaggaccgcatttgaggccctggctgccgtactgggcgggactcagtccctgcacaccaactcctatgacgaagccctggcccttcccagcgacgagtcggtgcttattgcattgcgcacacagcaggtgatcggctatgaaatcggcgtttgcgacgtggttgacccgcttggcggatcctactacattgaaagcctgaccaaccagcttgaagcaaaagcctgggagtacattgagaagattgatgccctcggcggtgccgtaaaggccatcgattacatgcagaaggagatccacaacgccgcttaccagtatcaactggctattgacaataagaagaagaccgttatcggagtgaacaaattccagttgaaggaagaagaaaagccaaagaacctgctgaaagtggacctctccgtgggcgaacggcagattgcgaagctcaaaaagcttaaggaagaaagagataacgccaaggttgaagccctgctgaaacaagtgcgcgaggcggcgcagagcgatgcaaacatgatgcctgtctttatcgatgcggttaaggaatacgttactctgggcgagatctgcggcgtcctgagagacgtattcggcgaatacaagcagcaaatcgtattctag

SEQ ID NO: 107 - protein, 652 aa, Artificial Sequence
  Description of Artificial Sequence Synthetic polypeptide
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 108 - DNA, 1959 bp, Artificial Sequence
  Description of Artificial Sequence Synthetic polynucleotide
atgagccaaattcacaaacacaccattcctgccaacatcgcagaccgttgcctgataaaccctcagcagtacgaggcgatgtatcaacaatctattaacgtacctgataccttctggggcgaacagggaaaaattcttgactggatcaaaccttaccagaaggtgaaaaacacctcctttgcccccggtaatgtgtccattaaatggtacgaggacggcacgctgaatctggcggcaaactgccttgaccgccatctgcaagaaaacggcgatcgtaccgccatcatctgggaaggcgacgacgccagccagagcaaacatatcagctataaagagctgcaccgcgacgtctgccgcttcgccaataccctgctcgagctgggcattaaaaaaggtgatgtggtggcgatttatatgccgatggtgccggaagccgcggttgcgatgctggcctgcgcccgcattggcgcggtgcattcggtgattttcggcggcttctcgccggaagccgttgccgggcgcattattgattccaactcacgactggtgatcacttccgacgaaggtgtgcgtgccgggcgcagtattccgctgaagaaaaacgttgatgacgcgctgaaaaacccgaacgtcaccagcgtagagcatgtggtggtactgaagcgtactggcgggaaaattgactggcaggaagggcgcgacctgtggtggcacgacctggttgagcaagcgagcgatcagcaccaggcggaagagatgaacgccgaagatccgctgtttattctctacacctccggttctaccggtaagccaaaaggtgtgctgcatactaccggcggttatctggtgtacgcggcgctgacctttaaatatgtctttgattatcatccgggtgatatctactggtgcaccgccgatgtgggctgggtgaccggacacagttacttgctgtacggcccgctggcctgcggtgcgaccacgctgatgtttgaaggcgtacccaactggccgacgcctgcccgtatggcgcaggtggtggacaagcatcaggtcaatattctctataccgcacccacggcgatccgcgcgctgatggcggaaggcgataaagcgatcgaaggcaccgaccgttcgtcgctgcgcattctcggttccgtgggcgagccaattaacccggaagcgtgggagtggtactggaaaaaaatcggcaacgagaaatgtccggtggtcgatacctggtggcagaccgaaaccggcggtttcatgatcaccccgctgcctggcgctaccgagctgaaagccggttcggcaacacgtccgttcttcggcgtgcaaccggcgctggtcgataacgaaggtaacccgctggagggggccaccgaaggtagcctggtaatcaccgactcctggccgggtcaggcgcgtacgctgtttggcgatcacgaacgttttgaacagacctacttctccaccttcaaaaatatgtatttcagcggcgacggcgcgcgtcgcgatgaagatggctattactggataaccgggcgtgtggacgacgtgctgaacgtctccggtcaccgtctggggacggcagagattgagtcggcgctggtggcgcatccgaagattgccgaagccgccgtagtaggtattccgcacaatattaaaggtcaggcgatctacgcctacgtcacgcttaatcacggggaggaaccgtcaccagaactgtacgcagaagtccgcaactgggtgcgtaaagagattggcccgctggcgacgccagacgtgctgcactggaccgactccctgcctaaaacccgctccggcaaaattatgcgccgtattctgcgcaaaattgcggcgggcgataccagcaacctgggcgatacctcgacgcttgccgatcctggcgtagtcgagaagctgcttgaagagaagcaggctatcgcgatgccatcgtaa

SEQ ID NO: 109 - protein, 638 aa, Artificial Sequence
  Description of Artificial Sequence Synthetic polypeptide
MSSTDQGTNPADTDDLTPTTLSLAGDFPKATEEQWEREVEKVLNRGRPPEKQLTFAECLKRLTVHTVDGIDIVPMYRPKDAPKKLGYPGVAPFTRGTTVRNGDMDAWDVRALHEDPDEKFTRKAILEGLERGVTSLLLRVDPDAIAPEHLDEVLSDVLLEMTKVEVFSRYDQGAAAEALVSVYERSDKPAKDLALNLGLDPIAFAALQGTEPDLTVLGDWVRRLAKFSPDSRAVTIDANIYHNAGAGDVAELAWALATGAEYVRALVEQGFTATEAFDTINFRVTATHDQFLTIARLRALREAWARIGEVFGVDEDKRGARQNAITSWRDVTREDPYVNILRGSIATFSASVGGAESITTLPFTQALGLPEDDFPLRIARNTGIVLAEEVNIGRVNDPAGGSYYVESLTRSLADAAWKEFQEVEKLGGMSKAVMTEHVTKVLDACNAERAKRLANRKQPITAVSEFPMIGARSIETKPFPAAPARKGLAWHRDSEVFEQLMDRSTSVSERPKVFLACLGTRRDFGGREGFSSPVWHIAGIDTPQVEGGTTAEIVEAFKKSGAQVADLCSSAKVYAQQGLEVAKALKAAGAKALYLSGAFKEFGDDAAEAEKLIDGRLFMGMDVVDTLSSTLDILGVAK

SEQ ID NO: 110 - DNA, 1869 bp, Artificial Sequence
  Description of Artificial Sequence Synthetic polynucleotide
tcaggcctccagcttgtccagggtggaggtcagcaactccaccacattcattccgtcgaagacgttgccgtcgatcacggcgttcacctcggcctcgtcgccgccgagttccttcagctgcccggcgagccgcacctcctgggcgcccgcctccttcagggccttcgcgacggcgagaccgtgggcggcgtagaccttcgcgctggagcacaggaccgcgatgtcggtgcccgcctcctgcatggccttgacgaacacctccgggttggtgccctccgcgatcacggtgttgatgccacccacgtggtacaggttcgaggtgaagccctcgcgaccaccgaagtcgcgccgggtgccgaggcaggccagcagcacggtcggggtcttcttggcggccttggaacggtcccggaggtcctcgaagacctggctgtcgcgcacgaacgggatgccgccgagcttcggggccgcggggcgcggggcgcgttcgagggccttctcgaggtggttcgggaacatcgagacgcccgtcagcggcagcttgcgggtggcgagcagcttggcgcgggcctcgttgatttccttgagctgggcggccacggtgccatcggcgatggcggcagccatacccttctcgtcgagctgaccgaacagctcccaggccttctcgcagagctgcttggtcatggactcgacgaaccatgcgccgccggccgggtcgttgacgcggccgatgttcgactcctcggccagcaccacctgggtgttgcgggcgatacggcgggtcaggacgtcgggaagaccgatcacggtgtcgagcggcaacacggtgatgaactcggcctggccgacggccgcggcgaaggccgagatggtgccgcgcagcacgttgacgtaggcgtcgtcgcgggtgatctcgcgcagcgacgtgacggcgtgctgcaccgcgccgcgtttctccgggctcaccccgagcacctcgccgacccggttccacagggtgcgcagcgcgcgcagccgggagatggtgatgaactggttggtgttcgcggagacccggaacaggatgctgtcgaaggcctcgtcggcgctcagcccgagatcggtgagggcgcgcacgtactcgatgcccgtggccagcgcgtaggccagctgggcgacgtcaccggcgcccatggaatcgtaacgggaggcgtccacgacgatgggacgcacgcccgagaacggctttgcaagctccacggccctcgcgatcaccgagaggtcgggggtggttccgttgagggcggcgaaaccgatcgggtcgatgccgaggctgccgcggatgttctccttaccggaggcggcgaaagccgcggccagggcctcggcggccgccagctcatcggtgttggaggaaacatgtgtgggggcaaggtcgaacaggacatcgctcaggacctcagccagcttgtctgcggggaccgcatcgggatccacgcgcacccagaccgcggaggtgccgcgctccaggtcggtgtccacggccttgcgggcctcggccgggtcgggttcctcaatgagctgagcactgaaccagccctcatccatctctcctgcacggaccgtggtgccgcgcgtgaatggggccactccggggaaaccaagctccttgacgccatcgtcaatggtgtacagcggcttgatcacaagcccatcgacggtgtggctcgtcaggcgcttgtatgcctgctcaatgttgagttccttgccctcgggacgcctccggttcagtaccttcagcacctctttctcccagtctgcaaggctgggagtggcgaagtcagcggcgagactgatctcggccgcgctcgttgattctgcgctcat

SEQ ID NO: 111 - protein, 728 aa, Artificial Sequence
  Description of Artificial Sequence Synthetic polypeptide
MSTLPRFDSVDLGNAPVPADAARRFEELAAKAGTGEAWETAEQIPVGTLFNEDVYKDMDWLDTYAGIPPFVHGPYATMYAFRPWTIRQYAGFSTAKESNAFYRRNLAAGQKGLSVAFDLPTHRGYDSDNPRVAGDVGMAGVAIDSIYDMRELFAGIPLDQMSVSMTMNGAVLPILALYVVTAEEQGVKPEQLAGTIQNDILKEFMVRNTYIYPPQPSMRIISEIFAYTSANMPKWNSISISGYHMQEAGATADIEMAYTLADGVDYIRAGESVGLNVDQFAPRLSFFWGIGMNFFMEVAKLRAARMLWAKLVHQFGPKNPKSMSLRTHSQTSGWSLTAQDVYNNVVRTCIEAMAATQGHTQSLHTNSLDEAIALPTDFSARIARNTQLFLQQESGTTRVIDPWSGSAYVEELTWDLARKAWGHIQEVEKVGGMAKAIEKGIPKMRIEEAAARTQARIDSGRQPLIGVNKYRLEHEPPLDVLKVDNSTVLAEQKAKLVKLRAERDPEKVKAALDKITWAAGNPDDKDPDRNLLKLCIDAGRAMATVGEMSDALEKVFGRYTAQIRTISGVYSKEVKNTPEVEEARELVEEFEQAEGRRPRILLAKMGQDGHDRGQKVIATAYADLGFDVDVGPLFQTPEETARQAVEADVHVVGVSSLAGGHLTLVPALRKELDKLGRPDILITVGGVIPEQDFDELRKDGAVEIYTPGTVIPESAISLVKKLRASLDA

SEQ ID NO: 112 - DNA, 2187 bp, Artificial Sequence
  Description of Artificial Sequence Synthetic polynucleotide
gtgagcactctgccccgttttgattcagttgacctcggcaatgccccggttcctgctgatgccgcacgacgcttcgaggaactggccgccaaggccggcaccggagaggcgtgggagacggccgagcagattccggttggcaccctgttcaacgaagacgtctacaaggacatggactggctggacacctacgcaggtatcccgccgttcgtccacggcccgtatgcaaccatgtacgcgttccgtccctggacgattcgccagtacgccggtttctccacggccaaggagtcgaacgccttctaccgccgcaaccttgcggccggccagaagggcctgtcggttgccttcgacctgcccacccaccgtggctacgactcggacaatccccgcgtcgccggtgacgtcggcatggccggtgtggccatcgactccatctatgacatgcgcgagctgttcgccggcattccgctggaccagatgagcgtgtccatgaccatgaacggcgccgtgctgccgatcctggccctctatgtggtgaccgccgaggagcagggcgtcaagcccgagcagctcgccgggacgatccagaacgacatcctcaaggagttcatggttcgtaacacctacatctacccgccgcagccgagtatgcgaatcatctctgagatcttcgcctacacgagtgccaatatgccgaagtggaattcgatttccatttccggctaccacatgcaggaagccggcgccacggccgacatcgagatggcctataccctggccgacggtgttgactacatccgcgccggcgagtcggtgggcctcaatgtcgaccagttcgcgccgcgtctgtccttcttctggggcatcggcatgaacttcttcatggaggttgccaagctgcgtgccgcgcgcatgttgtgggccaagctggtgcatcagttcgggccgaagaacccgaagtcgatgagcctgcgcacccactcgcagacctccggttggtcgctgaccgcccaggacgtctacaacaacgtcgtgcgtacctgcatcgaggccatggccgccacccagggccatacccagtcgctgcacacgaactcgctcgacgaggccatcgccctgccgaccgatttcagcgcccgcatcgcccgtaacacccagctgttcctgcagcaggaatcgggcacgacgcgcgtgatcgacccgtggagcggctcggcatacgtcgaggagctcacctgggacctggcccgcaaggcatggggtcacatccaggaggtcgagaaggtcggcggcatggccaaggccatcgaaaagggcatccccaagatgcgcatcgaggaagccgccgcccgcacccaggcacgcatcgactccggccgccagccgctgatcggcgtgaacaagtaccgcctggagcacgagccgccgctcgatgtgctcaaggtggacaactccacggtgctcgccgagcagaaggccaagctggtcaagctgcgcgccgagcgcgatcccgagaaggtcaaggccgccctcgacaagatcacctgggccgccggcaaccccgacgacaaggatccggatcgcaacctgctgaagctgtgcatcgacgctggccgcgccatggcgacggtcggcgagatgagcgacgcgctcgagaaggtcttcggacgctacaccgcccagattcgcaccatctccggtgtgtactcgaaggaagtgaagaacacgcctgaggttgaggaagcacgcgagctcgttgaggaattcgagcaggccgagggccgtcgtcctcgcatcctgctggccaagatgggccaggacggtcacgaccgtggccagaaggtcatcgccaccgcctatgccgacctcggtttcgacgtcgacgtgggcccgctgttccagaccccggaggagaccgcacgtcaggccgtcgaggccgatgtgcacgtggtgggcgtttcgtcgctcgccggcgggcatctgacgctggttccggccctgcgcaaggagctggacaagctcggacgtcccgacatcctcatcaccgtgggcggcgtgatccctgagcaggacttcgacgagctgcgtaaggacggcgccgtggagatctacacccccggcaccgtcattccggagtcggcgatctcgctggtcaagaaactgcgggcttcgctcgatgcctag

SEQ ID NO: 113 - protein, 242 aa, Artificial Sequence
  Description of Artificial Sequence Synthetic polypeptide
MEKPRVLVLTGAGISAESGIRTFRAADGLWEEHRVEDVATPEGFDRDPELVQAFYNARRRQLQQPEIQPNAAHLALAKLQDALGDRFLLVTQNIDNLHERAGNTNVIHMHGELLKVRCSQSGQVLDWTGDVTPEDKCHCCQFPAPLRPHVVWFGEMPLGMDEIYMALSMADIFIAIGTSGHVYPAAGFVHEAKLHGAHTVELNLEPSQVGNEFAEKYYGPASQVVPEFVEKLLKGLKAGSIA

SEQ ID NO: 114 - DNA, 729 bp, Artificial Sequence
  Description of Artificial Sequence Synthetic polynucleotide
atggaaaaaccaagagtactcgtactgacaggggcaggaatttctgcggaatcaggtattcgtacctttcgcgccgcagatggcctgtgggaagaacatcgggttgaagatgtggcaacgccggaaggtttcgatcgcgatcctgaactggtgcaagcgttttataacgcccgtcgtcgacagctgcagcagccagaaattcagcctaacgccgcgcatcttgcgctggctaaactgcaagacgccctcggcgatcgctttttgctggtgacgcagaatatcgacaacctgcatgaacgcgcaggtaataccaatgtgattcatatgcatggggaactgctgaaagtgcgttgttcacaaagtggtcaggttctcgactggacaggagacgttaccccagaagataaatgccattgttgccagtttccggcacccttgcgcccgcacgtagtgtggtttggcgaaatgccactcggcatggatgaaatttatatggcgttgtcgatggccgatattttcattgccattggcacatccgggcatgtttatccggcggctgggtttgttcacgaagcgaaactgcatggcgcgcacaccgtggaactgaatcttgaaccgagtcaggttggtaatgaatttgccgagaaatattacggcccggcaagccaggtggtgcctgagtttgttgaaaagttgctgaagggattaaaagcgggaagcattgcctga

SEQ ID NO: 115 - protein, 886 aa, Artificial Sequence
  Description of Artificial Sequence Synthetic polypeptide
MSQRGLEALLRPKSIAVIGASMKPNRAGYLMMRNLLAGGFNGPVLPVTPAWKAVLGVLAWPDIASLPFTPDLAVLCTNASRNLALLEELGEKGCKTCIILSAPASQHEDLRACALRHNMRLLGPNSLGLLAPWQGLNASFSPVPIKRGKLAFISQSAAVSNTILDWAQQRKMGFSYFIALGDSLDIDVDELLDYLARDSKTSAILLYLEQLSDARRFVSAARSASRNKPILVIKSGRSPAAQRLLNTTAGMDPAWDAAIQRAGLLRVQDTHELFSAVETLSHMRPLRGDRLMIISNGAAPAALALDALWSRNGKLATLSEETCQKLRDALPEHVAISNPLDLRDDASSEHYIKTLDILLHSQDFDALMVIHSPSAAAPATESAQVLIEAVKHHPRSKYVSLLTNWCGEHSSQEARRLFSEAGLPTYRTPEGTITAFMHMVEYRRNQKQLRETPALPSNLTSNTAEAHLLLQQAIAEGATSLDTHEVQPILQAYGMNTLPTWIASDSTEAVHIAEQIGYPVALKLRSPDIPHKSEVQGVMLYLRTANEVQQAANAIFDRVKMAWPQARVHGLLVQSMANRAGAQELRVVVEHDPVFGPLIMLGEGGVEWRPEDQAVVALPPLNMNLARYLVIQGIKSKKIRARSALRPLDVAGLSQLLVQVSNLIVDCPEIQRLDIHPLLASGSEFTALDVTLDISPFEGDNESRLAVRPYPHQLEEWVELKNGERCLFRPILPEDEPQLQQFISRVTKEDLYYRYFSEINEFTHEDLANMTQIDYDREMAFVAVRRIDQTEEILGVTRAISDPDNIDAEFAVLVRSDLKGLGLGRRLMEKLITYTRDHGLQRLNGITMPNNRGMVALARKLGFNVDIQLEEGIVGLTLNLAQREES

SEQ ID NO: 116 - DNA, 2661 bp, Artificial Sequence
  Description of Artificial Sequence Synthetic polynucleotide
atgagtcagcgaggactggaagcactactgcgaccaaaatcgatagcggtaattggcgcgtcgatgaaacccaatcgcgcaggttacctgatgatgcgtaacctgctggcgggaggctttaacggaccggtactcccggtgacgccagcctggaaagcggtgttgggtgtgttggcctggccggatattgccagcttgccctttacacccgaccttgcggttttatgtaccaatgccagccgtaatcttgctcttctggaagagctcggcgagaaaggctgtaaaacctgcattattctttccgccccggcatcgcaacacgaagatctccgcgcctgcgccctgcgccataacatgcgcctgcttggaccaaacagtctgggtttactggctccctggcaaggtctgaatgccagcttttcgcctgtgccgattaaacgcggcaagctggcgtttatttcgcaatcggctgccgtctccaacaccatcctcgactgggcgcaacagcgtaagatgggcttttcctactttattgcgctcggcgacagcctggatatcgacgttgatgaattgcttgactatctggcacgcgacagtaaaaccagcgccatcctgctctatctcgaacagttaagcgacgcgcgacgctttgtttcggcggcccgtagtgcctcgcgtaataaaccgattctggtgattaaaagcggacgtagcccggcggcacagcgactgctcaacacgacggcaggaatggacccggcatgggatgcggctattcagcgtgccggtttgttgcgggtacaggacacccacgagctgttttcggcggtggaaacccttagccatatgcgcccgctacgtggcgaccggctgatgattatcagcaacggtgctgcgcctgccgcgctggcgctggatgccttatggtcacgcaatggcaagctggcaacgctaagcgaagaaacctgccagaaactgcgcgatgcactgccagaacatgtggcaatatctaacccgctcgatctacgcgatgacgccagcagtgagcactatattaaaacgctggatattctgctccacagccaggattttgacgcgctgatggttattcattcgcccagcgccgctgctcccgcaacagaaagcgcgcaagtattaattgaagcggtaaagcatcatccccgcagcaaatatgtctctttgctgacgaactggtgcggcgagcactcctcgcaagaggcacgacgtttattcagcgaagccgggctgccgacctaccgtaccccggaaggaaccatcactgcttttatgcatatggtggagtaccggcgtaatcagaagcaactacgcgaaacgccggcgttgcccagcaatctgacttccaataccgcagaagcgcatcttctgttgcaacaggcgattgccgaaggggctacgtcgctcgatacccatgaagttcagcccatcctgcaagcgtatggcatgaacacgctccctacctggattgccagcgatagcaccgaagcggtgcatattgccgaacagattggttatccggtggcgctgaaattgcgttcgccggatattccacataaatcggaagttcagggcgtcatgctttacctgcgtacagccaatgaagtccagcaagcggcgaacgctattttcgatcgcgtaaaaatggcctggccacaggcgcgggtccacggcctgttggtgcaaagtatggctaaccgtgctggcgctcaggagttgcgggttgtggttgagcacgatccggttttcgggccgttgatcatgctgggtgaaggcggtgtggagtggcgtcctgaagatcaagccgtcgtcgcactgccgccgctgaacatgaacctggcccgctatctggttattcaggggatcaaaagtaaaaagattcgtgcgcgcagtgcgctacgcccattggatgttgcaggcttgagccagcttctggtgcaggtttccaacttgattgtcgattgcccggaaattcagcgtctggatattcatcctttgctggcttctggcagtgaatttaccgcgctggatgtcacgctggatatctcgccgtttgaaggcgataacgagagtcggctggcagtgcgcccttatccgcatcagctggaagaatgggtagaattgaaaaacggtgaacgctgcttgttccgcccgattttgccagaagatgagccacaacttcagcaattcatttcgcgagtcaccaaagaagatctttattaccgctactttagcgagatcaacgaatttacccatgaagatttagccaacatgacacagatcgactacgatcgggaaatggcgtttgtagcggtacgacgtattgatcaaacggaagagatcctcggcgtcacgcgtgcgatttccgatcctgataacatcgatgccgaatttgctgtactggttcgctcggatctcaaagggttaggcttaggtcgacgcttaatggaaaagttgattacctatacgcgagatcacggactacaacgtctgaatggtattacgatgccaaacaatcgtggcatggtggcgctagcccgcaagctcgggtttaacgttgatatccagctcgaagaggggatcgttgggcttacgctaaatcttgcccagcgcgaggaatcatga

SEQ ID NO: 117 - protein, 461 aa, Artificial Sequence
  Description of Artificial Sequence Synthetic polypeptide
MLTFIELLIGVVVIVGVARYIIKGYSATGVLFVGGLLLLIISAIMGHKVLPSSQASTGYSATDIVEYVKILLMSRGGDLGMMIMMLCGFAAYMTHIGANDMVVKLASKPLQYINSPYLLMIAAYFVACLMSLAVSSATGLGVLLMATLFPVMVNVGISRGAAAAICASPAAIILAPTSGDVVLAAQASEMSLIDFAFKTTLPISIAAIIGMAIAHFFWQRYLDKKEHISHEMLDVSEITTTAPAFYAILPFTPIIGVLIFDGKWGPQLHIITILVICMLIASILEFLRSFNTQKVFSGLEVAYRGMADAFANVVMLLVAAGVFAQGLSTIGFIQSLISIATSFGSASIILMLVLVILTMLAAVTTGSGNAPFYAFVEMIPKLAHSSGINPAYLTIPMLQASNLGRTLSPVSGVVVAVAGMAKISPFEVVKRTSVPVLVGLVIVIVATELMVPGTAAAVTGK

SEQ ID NO: 118 - DNA, 1386 bp, Artificial Sequence
  Description of Artificial Sequence Synthetic polynucleotide
atgctgacattcattgagctccttattggggttgtggttattgtgggtgtagctcgctacatcattaaagggtattccgccactggtgtgttatttgtcggtggcctgttattgctgattatcagtgccattatggggcacaaagtgttaccgtccagccaggcttcaacaggctacagcgccacggatatcgttgaatacgttaaaatattactaatgagccgcggcggcgacctcggcatgatgattatgatgctgtgtggatttgccgcttacatgacccatatcggcgcgaatgatatggtggtcaagctggcgtcaaaaccattgcagtatattaactccccttacctgctgatgattgccgcctattttgtcgcctgtctgatgtctctggccgtctcttccgcaaccggtctgggtgttttgctgatggcaaccctatttccggtgatggtaaacgttggtatcagtcgtggcgcagctgctgccatttgtgcctccccggcggcgattattctcgcaccgacttcaggggatgtggtgctggcggcgcaagcttccgaaatgtcgctgattgacttcgccttcaaaacgacgctgcctatctcaattgctgcaattatcggcatggcgatcgcccacttcttctggcaacgttatctggataaaaaagagcacatctctcatgaaatgttagatgtcagtgaaatcaccaccactgctcctgcgttttatgccattttgccgttcacgccgatcatcggtgtactgatttttgacggtaaatggggtccgcaattacacatcatcactattctggtgatttgtatgctgattgcctccattctggagttcctccgcagctttaatacccagaaagttttctctggtctggaagtggcttatcgcgggatggcagatgcgtttgctaacgtggtgatgctgctggttgccgctggggtattcgctcaggggcttagcaccatcggctttattcaaagtctgatttctatcgctacctcgtttggttcggcgagtatcatcctgatgctggtattggtgattctgacaatgctggcggcagtcacgaccggttcaggcaatgcgccgttttatgcgtttgttgagatgatcccgaaactggcgcactcttccggcattaacccggcgtatttgactatcccgatgctgcaggcgtcaaaccttggccgtaccctttcgcccgtttctggcgtagtcgttgcggttgccgggatggcgaagatctcgccgtttgaagtcgtaaaacgcacctcggtaccggtgcttgttggtttggtgattgttatcgttgctacagagctgatggtgccaggaacggcagcagcggtcacaggcaagtaa

SEQ ID NO: 119 - protein, 524 aa, Artificial Sequence
  Description of Artificial Sequence Synthetic polypeptide
MTVGLLLGRIKIFGFRLGVAAVLFVGLALSTIEPDISVPSLIYVVGLSLFVYTIGLEAGPGFFTSMKTTGLRNNALTLGAIIATTALAWALITVLNIDAASGAGMLTGALTNTPAMAAVVDALPSLIDDTGQLHLIAELPVVAYSLAYPLGVLIVILSIAIFSSVFKVDHNKEAEEAGVAVQELKGRRIRVTVADLPALENIPELLNLHVIVSRVERDGEQFIPLYGEHARIGDVLTVVGADEELNRAEKAIGELIDGDPYSNVELDYRRIFVSNTAVVGTPLSKLQPLFKDMLITRIRRGDTDLVASSDMTLQLGDRVRVVAPAEKLREATQLLGDSYKKLSDFNLLPLAAGLMIGVLVGMVEFPLPGGSSLKLGNAGGPLVVALLLGMINRTGKFVWQIPYGANLALRQLGITLFLAAIGTSAGAGFRSAISDPQSLTIIGFGALLTLFISITVLFVGHKLMKIPFGETAGILAGTQTHPAVLSYVSDASRNELPAMGYTSVYPLAMIAKILAAQTLLFLLI

SEQ ID NO: 120 - DNA, 1620 bp, Artificial Sequence
  Description of Artificial Sequence Synthetic polynucleotide
gtgagcttccttgtagaaaatcaattactcgcgttggttgtcatcatgacggtcggactattgctcggccgcatcaaaattttcgggttccgtctcggcgtcgccgctgtactgtttgtaggtctagcgctatccaccattgagccggatatttccgtcccatccctcatttacgtggttggactgtcgctttttgtctacacgatcggtctggaagccggccctggattcttcacctccatgaaaaccactggtctgcgcaacaacgcactgaccttgggcgccatcatcgccaccacggcactcgcatgggcactcatcacagttttgaacatcgatgccgcctccggcgccggcatgctcaccggcgcgctcaccaacaccccagccatggccgcagttgttgacgcacttccttcgcttatcgacgacaccggccagcttcacctcatcgccgagctgcccgtcgtcgcatattccttggcataccccctcggtgtgctcatcgttattctctccatcgccatcttcagctctgtgttcaaagtcgaccacaacaaagaagccgaagaagcgggcgttgcggtccaggaactcaaaggccgtcgcatccgcgtcaccgtcgctgatcttccagccctggagaacatcccagagctgctcaacctccacgtcattgtgtcccgagtggaacgagacggtgagcaattcatcccgctttatggcgaacacgcacgcatcggcgatgtcttaacagtggtgggtgccgatgaagaactcaaccgcgcggaaaaagccatcggtgaactcattgacggcgacccctacagcaatgtggaacttgattaccgacgcatcttcgtctcaaacacagcagtcgtgggcactcccctatccaagctccagccactgtttaaagacatgctgatcacccgcatcaggcgcggcgacacagatttggtggcctcctccgacatgactttgcagctcggtgaccgtgtccgcgttgtcgcaccagcagaaaaactccgcgaagcaacccaattgctcggcgattcctacaagaaactctccgatttcaacctgctcccactcgctgccggcctcatgatcggtgtgcttgtcggcatggtggagttcccactaccaggtggaagctccctgaaactgggtaacgcaggtggaccgctagttgttgcgctgctgctcggcatgatcaatcgcacaggcaagttcgtctggcaaatcccctacggagcaaaccttgcccttcgccaactgggcatcacactatttttggctgccatcggtacctcagcgggcgcaggatttcgatcagcgatcagcgacccccaatcactcaccatcatcggcttcggtgcgctgctcactttgttcatctccatcacggtgctgttcgttggccacaaactgatgaaaatccccttcggtgaaaccgctggcatcctcgccggtacgcaaacccaccctgctgtgctgagttatgtgtcagatgcctcccgcaacgagctccctgccatgggttatacctctgtgtatccgctggcgatgatcgcaaagatcctggccgcccaaacgttgttgttcctacttatctag

SEQ ID NO: 121 - protein, 559 aa, Artificial Sequence
  Description of Artificial Sequence Synthetic polypeptide
MNKIFSSHVMPFRALIDACWKEKYTAARFTRDLIAGITVGIIAIPLAMALAIGSGVAPQYGLYTAAVAGIVIALTGGSRFSVSGPTAAFVVILYPVSQQFGLAGLLVATLLSGIFLILMGLARFGRLIEYIPVSVTLGFTSGIGITIGTMQIKDFLGLQMAHVPEHYLQKVGALFMALPTINVGDAAIGIVTLGILVFWPRLGIRLPGHLPALLAGCAVMGIVNLLGGHVATIGSQFHYVLADGSQGNGIPQLLPQLVLPWDLPNSEFTLTWDSIRTLLPAAFSMAMLGAIESLLCAVVLDGMTGTKHKANSELVGQGLGNIIAPFFGGITATAAIARSAANVRAGATSPISAVIHSILVILALLVLAPLLSWLPLSAMAALLLMVAWNMSEAHKVVDLLRHAPKDDIIVMLLCMSLTVLFDMVIAISVGIVLASLLFMRRIARMTRLAPVVVDVPDDVLVLRVIGPLFFAAAEGLFTDLESRLEGKRIVILKWDAVPVLDAGGLDAFQRFVKRLPEGCELRVCNVEFQPLRTMARAGIQPIPGRLAFFPNRRAAMADL

SEQ ID NO: 122 - DNA, 1680 bp, Artificial Sequence
  Description of Artificial Sequence Synthetic polynucleotide
gtgaacaaaatattttcctcacatgtgatgcctttccgcgctctgatcgacgcttgctggaaagaaaaatatactgccgcacggtttacccgtgacctgattgccgggataaccgtcgggattattgctatcccgctggcgatggcgttggctattggtagtggtgtggcaccccagtacggtttatataccgcagctgttgcggggattgtcattgctctgacgggtgggtcacgctttagcgtttccggtccgactgcggcatttgtggtaattctctatcccgtgtcgcaacagtttggactggcaggactgctggttgcgaccttgctgtcggggatctttttgattctgatgggtctggcacgctttggtcgcctgattgagtatattccggtttccgtcaccttaggtttcacctcgggtatcgggatcaccatcggtaccatgcagattaaagattttctcggtctgcaaatggcccatgtcccggaacattatctacaaaaagtcggcgcattatttatggcgctgccgaccattaatgtgggtgatgctgccattggcattgtgacgctaggtattcttgttttttggccgcgtctgggcattcgtttacccggtcaccttccggccttgctggctggttgcgcggtgatggggattgttaacctgctcggcggacatgttgctaccatcggttcgcaattccactacgtcctggccgatggttctcagggtaacggtattccgcaactgctgccgcaactggtgctgccgtgggatctgcctaattcagaattcacgctaacctgggattctattcgcacactgctgcctgcggcattctcaatggcaatgctcggcgcaatcgaatctctgctctgcgccgtggtgctggatggtatgaccgggacgaaacacaaggcgaacagcgaactggttggacagggactggggaatattatcgctccgttctttggtggtattaccgctacagctgccatcgcgcgttctgccgctaacgtccgtgccggggcaacgtcccctatctcggcggtgatccactctattctggttattcttgccctgctggtactggcaccgctgctctcctggctgccgctttccgccatggcagccctgctgttgatggtggcgtggaacatgagtgaagcgcacaaagtggtcgacttgctgcgtcatgcgccgaaagatgacatcatcgtcatgctgctgtgcatgtcgctgaccgtgttgtttgatatggttattgccatcagcgtggggatcgtgctggcatcgctgctgtttatgcgtcgtatcgcacgtatgactcgcctggcaccggtagtcgtagatgttccagacgatgtcctggttctgcgcgttattggcccgctgttttttgctgctgctgaaggcttattcacggacctggagtcacgtcttgaaggcaaacggattgtgattctgaagtgggatgccgttccggtacttgatgctggtggtcttgatgcgttccagcgttttgtgaagcgtctgcccgagggatgtgaactgcgcgtgtgcaacgtggaattccagccactgcgcactatggctcgcgctggcattcaaccgatcccgggacgcctggcgttcttcccgaatcgtcgcgcggcgatggcggatttataa

SEQ ID NO: 123 - protein, 428 aa, Artificial Sequence
  Description of Artificial Sequence Synthetic polypeptide
MKTSLFKSLYFQVLTAIAIGILLGHFYPEIGEQMKPLGDGFVKLIKMIIAPVIFCTVVTGIAGMESMKAVGRTGAVALLYFEIVSTIALIIGLIIVNVVQPGAGMNVDPATLDAKAVAVYADQAKDQGIVAFIMDVIPASVIGAFASGNILQVLLFAVLFGFALHRLGSKGQLIFNVIESFSQVIFGIINMIMRLAPIGAFGAMAFTIGKYGVGTLVQLGQLIICFYITCILFVVLVLGSIAKATGFSIFKFIRYIREELLIVLGTSSSESALPRMLDKMEKLGCRKSVVGLVIPTGYSFNLDGTSIYLTMAAVFIAQATNSQMDIVHQITLLIVLLLSSKGAAGVTGSGFIVLAATLSAVGHLPVAGLALILGIDRFMSEARALTNLVGNGVATIVVAKWVKELDHKKLDDVLNNRAPDGKTHELSS

SEQ ID NO: 124 - DNA, 1287 bp, Artificial Sequence
  Description of Artificial Sequence Synthetic polynucleotide
atgaaaacctctctgtttaaaagcctttactttcaggtcctgacagcgatagccattggtattctccttggccatttctatcctgaaataggcgagcaaatgaaaccgcttggcgacggcttcgttaagctcattaagatgatcatcgctcctgtcatcttttgtaccgtcgtaacgggcattgcgggcatggaaagcatgaaggcggtcggtcgtaccggcgcagtcgcactgctttactttgaaattgtcagtaccatcgcgctgattattggtcttatcatcgttaacgtcgtgcagcctggtgccggaatgaacgtcgatccggcaacgcttgatgcgaaagcggtagcggtttacgccgatcaggcgaaagaccagggcattgtcgccttcattatggatgtcatcccggcgagcgtcattggcgcatttgccagcggtaacattctgcaggtgctgctgtttgccgtactgtttggttttgcgctccaccgtctgggcagcaaaggccaactgatttttaacgtcatcgaaagtttctcgcaggtcatcttcggcatcatcaatatgatcatgcgtctggcacctattggtgcgttcggggcaatggcgtttaccatcggtaaatacggcgtcggcacactggtgcaactggggcagctgattatctgtttctacattacctgtatcctgtttgtggtgctggtattgggttcaatcgctaaagcgactggtttcagtatcttcaaatttatccgctacatccgtgaagaactgctgattgtactggggacttcatcttccgagtcggcgctgccgcgtatgctcgacaagatggagaaactcggctgccgtaaatcggtggtggggctggtcatcccgacaggctactcgtttaaccttgatggcacatcgatatacctgacaatggcggcggtgtttatcgcccaggccactaacagtcagatggatatcgtccaccaaatcacgctgttaatcgtgttgctgctttcttctaaaggggcggcaggggtaacgggtagtggctttatcgtgctggcggcgacgctctctgcggtgggccatttgccggtagcgggtctggcgctgatcctcggtatcgaccgctttatgtcagaagctcgtgcgctgactaacctggtcggtaacggcgtagcgaccattgtcgttgctaagtgggtgaaagaactggaccacaaaaaactggacgatgtgctgaataatcgtgcgccggatggcaaaacgcacgaattatcctcttaa

SEQ ID NO: 125 - protein, 244 aa, Artificial Sequence
  Description of Artificial Sequence Synthetic polypeptide
MRIDILIGHTSFFHQTSRDNFLHYLNEEEIKRYDQFHFVSDKELYILSRILLKTALKRYQPDVSLQSWQFSTCKYGKPFIVFPQLAKKIFFNLSHTIDTVAVAISSHCELGVDIEQIRDLDNSYLNISQHFFTPQEATNIVSLPRYEGQLLFWKMWTLKEAYIKYRGKGLSLGLDCIEFHLTNKKLTSKYRGSPVYFSQWKICNSFLALASPLITPKITIELFPMQSQLYHHDYQLIHSSNGQN

SEQ ID NO: 126 - DNA, 967 bp, Artificial Sequence
  Description of Artificial Sequence Synthetic polynucleotide
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 127 - DNA, 424 bp, Artificial Sequence
  Description of Artificial Sequence Synthetic polynucleotide
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 128 - protein, 539 aa, Artificial Sequence
  Description of Artificial Sequence Synthetic polypeptide
MSFLVENQLLALVVIMTVGLLLGRIKIFGFRLGVAAVLFVGLALSTIEPDISVPSLIYVVGLSLFVYTIGLEAGPGFFTSMKTTGLRNNALTLGAIIATTALAWALITVLNIDAASGAGMLTGALTNTPAMAAVVDALPSLIDDTGQLHLIAELPVVAYSLAYPLGVLIVILSIAIFSSVFKVDHNKEAEEAGVAVQELKGRRIRVTVADLPALENIPELLNLHVIVSRVERDGEQFIPLYGEHARIGDVLTVVGADEELNRAEKAIGELIDGDPYSNVELDYRRIFVSNTAVVGTPLSKLQPLFKDMLITRIRRGDTDLVASSDMTLQLGDRVRVVAPAEKLREATQLLGDSYKKLSDFNLLPLAAGLMIGVLVGMVEFPLPGGSSLKLGNAGGPLVVALLLGMINRTGKFVWQIPYGANLALRQLGITLFLAAIGTSAGAGFRSAISDPQSLTIIGFGALLTLFISITVLFVGHKLMKIPFGETAGILAGTQTHPAVLSYVSDASRNELPAMGYTSVYPLAMIAKILAAQTLLFLLI

SEQ ID NO: 129 - protein, 461 aa, Artificial Sequence
  Description of Artificial Sequence Synthetic polypeptide
MLTFIELLIGVVVIVGVARYIIKGYSATGVLFVGGLLLLIISAIMGHKVLPSSQASTGYSATDIVEYVKILLMSRGGDLGMMIMMLCGFAAYMTHIGANDMVVKLASKPLQYINSPYLLMIAAYFVACLMSLAVSSATGLGVLLMATLFPVMVNVGISRGAAAAICASPAAIILAPTSGDVVLAAQASEMSLIDFAFKTTLPISIAAIIGMAIAHFFWQRYLDKKEHISHEMLDVSEITTTAPAFYAILPFTPIIGVLIFDGKWGPQLHIITILVICMLIASILEFIRSFNTQKVFSGLEVAYRGMADAFANVVMLLVAAGVFAQGLSTIGFIQSLISIATSFGSASIILMLVLVILTMLAAVTTGSGNAPFYAFVEMIPKLAHSSGINPAYLTIPMLQASNLGRTLSPVSGVVVAVAGMAKISPFEVVKRTSVPVLVGLVIVIVATELMVPGTAAAVTGK

SEQ ID NO: 130 - protein, 590 aa, Artificial Sequence
  Description of Artificial Sequence Synthetic polypeptide
MRKVLIANRGEIAVRVARACRDAGIASVAVYADPDRDALHVRAADEAFALGGDTPATSYLDIAKVLKAARESGADAIHPGYGFLSENAEFAQAVLDAGLIWIGPPPHAIRDRGEKVAARHIAQRAGAPLVAGTPDPVSGADEVVAFAKEHGLPIAIKAAFGGGGRGLKVARTLEEVPELYDSAVREAVAAFGRGECFVERYLDKPRHVETQCLADTHGNVVVVSTRDCSLQRRHQKLVEEAPAPFLSEAQTEQLYSSSKAILKEAGYGGAGTVEFLVGMDGTIFFLEVNTRLQVEHPVTEEVAGIDLVREMFRIADGEELGYDDPALRGHSFEFRINGEDPGRGFLPAPGTVTLFDAPTGPGVRLDAGVESGSVIGPAWDSLLAKLIVTGRTRAEALQRAARALDEFTVEGMATAIPFHRTVVRDPAFAPELTGSTDPFTVHTRWIETEFVNEIKPFTTPADTETDEESGRETVVVEVGGKRLEVSLPSSLGMSLARTGLAAGARPKRRAAKKSGPAASGDTLASPMQGTIVKIAVEEGQEVQEGDLIVVLEAMKMEQPLNAHRSGTIKGLTAEVGASLTSGAAICEIKD

SEQ ID NO: 131 - protein, 530 aa, Artificial Sequence
  Description of Artificial Sequence Synthetic polypeptide
MSEPEEQQPDIHTTAGKLADLRRRIEEATHAGSARAVEKQHAKGKLTARERIDLLLDEGSFVELDEFARHRSTNFGLDANRPYGDGVVTGYGTVDGRPVAVFSQDFTVFGGALGEVYGQKIVKVMDFALKTGCPVVGINDSGGARIQEGVASLGAYGEIFRRNTHASGVIPQISLVVGPCAGGAVYSPAITDFTVMVDQTSHMFITGPDVIKTVTGEDVGFEELGGARTHNSTSGVAHHMAGDEKDAVEYVKQLLSYLPSNNLSEPPAFPEEADLAVTDEDAELDTIVPDSANQPYDMHSVIEHVLDDAEFFETQPLFAPNILTGFGRVEGRPVGIVANQPMQFAGCLDITASEKAARFVRTCDAFNVPVLTFVDVPGFLPGVDQEHDGIIRRGAKLIFAYAEATVPLITVITRKAFGGAYDVMGSKHLGADLNLAWPTAQIAVMGAQGAVNILHRRTIADAGDDAEATRARLIQEYEDALLNPYTAAERGYVDAVIMPSDTRRHIVRGLRQLRTKRESLPPKKHGNIPL

SEQ ID NO: 132 - protein, 148 aa, Artificial Sequence
  Description of Artificial Sequence Synthetic polypeptide
MSNEDLFICIDHVAYACPDADEASKYYQETFGWHELHREENPEQGVVEIMMAPAAKLTEHMTQVQVMAPLNDESTVAKWLAKHNGRAGLHHMAWRVDDIDAVSATLRERGVQLLYDEPKLGTGGNRINFMHPKSGKGVLIELTQYPKN

SEQ ID NO: 133 - protein, 638 aa, Artificial Sequence
  Description of Artificial Sequence Synthetic polypeptide
MSSTDQGTNPADTDDLTPTTLSLAGDFPKATEEQWEREVEKVLNRGRPPEKQLTFAECLKRLTVHTVDGIDIVPMYRPKDAPKKLGYPGVAPFTRGTTVRNGDMDAWDVRALHEDPDEKFTRKAILEGLERGVTSLLLRVDPDAIAPEHLDEVLSDVLLEMTKVEVFSRYDQGAAAEALVSVYERSDKPAKDLALNLGLDPIAFAALQGTEPDLTVLGDWVRRLAKFSPDSRAVTIDANIYHNAGAGDVAELAWALATGAEYVRALVEQGFTATEAFDTINFRVTATHDQFLTIARLRALREAWARIGEVFGVDEDKRGARQNAITSWRELTREDPYVNILRGSIATFSASVGGAESITTLPFTQALGLPEDDFPLRIARNTGIVLAEEVNIGRVNDPAGGSYYVESLTRSLADAAWKEFQEVEKLGGMSKAVMTEHVTKVLDACNAERAKRLANRKQPITAVSEFPMIGARSIETKPFPAAPARKGLAWHRDSEVFEQLMDRSTSVSERPKVFLACLGTRRDFGGREGFSSPVWHIAGIDTPQVEGGTTAEIVEAFKKSGAQVADLCSSAKVYAQQGLEVAKALKAAGAKALYLSGAFKEFGDDAAEAEKLIDGRLFMGMDVVDTLSSTLDILGVAK

SEQ ID NO: 134 - protein, 728 aa, Artificial Sequence; identical to SEQ ID NO: 111
  Description of Artificial Sequence Synthetic polypeptide

SEQ ID NO: 135 - protein, 248 aa, Artificial Sequence; identical to SEQ ID NO: 100
  Description of Artificial Sequence Synthetic polypeptide

SEQ ID NO: 136 - protein, 590 aa, Artificial Sequence; identical to SEQ ID NO: 101
  Description of Artificial Sequence Synthetic polypeptide

SEQ ID NO: 137 - protein, 392 aa, Artificial Sequence; identical to SEQ ID NO: 99
  Description of Artificial Sequence Synthetic polypeptide

SEQ ID NO: 138 - DNA, 3705 bp, Artificial Sequence; identical to SEQ ID NO: 83
  Description of Artificial Sequence Synthetic polynucleotide

SEQ ID NO: 139 - DNA, 3811 bp, Artificial Sequence
  Description of Artificial Sequence Synthetic polynucleotide
gatcaaaaaggttagcctcaagagggtcataaaaatgtcagagcagaaagtagctctggttaccggtgcgttaggtggtatcggaagtgagatctgccgccagcttgtgaccgccgggtacaagattatcgccaccgttgttccacgcgaagaagaccgcgaaaaacaatggttgcaaagtgaggggtttcaagactctgatgtgcgtttcgtattaacagatttaaacaatcacgaagctgcgacagcggcaattcaagaagcgattgccgccgaaggacgcgttgatgtattggtcaacaacgcggggatcacgcgcgatgctacatttaagaaaatgtcctatgagcaatggtcccaagtcatcgacacgaatttaaagactctttttaccgtgacccagccagtatttaataaaatgcttgaacagaagtctggccgcatcgtaaacattagctctgtcaatggtttaaaagggcaatttggtcaagccaactactcggcctcgaaagcagggattatcgggtttactaaagcattggcgcaggagggtgctcgctcgaacatttgcgtcaatgtcgttgctcctggttacacagcgacacccatggtcacagcaatgcgcgaggatgtaattaagtcaatcgaagctcaaattcccctgcaacgtctggcagcaccggcggagattgcggcagcggttatgtatttggtgagtgaacacggtgcatacgtgacgggcgaaactttgagtatcaacggcgggctgtacatgcactaaaggtgcttttagtctagcgctagagcaggtaccatattaatgaatccaaattcctttcagtttaaagagaatatcttacagtttttcagcgtgcacgacgatatttggaaaaaactgcaggaattttactatggacaatcgcccatcaatgaagcgttggcgcagttaaataaggaagacatgagtttattcttcgaggcgttatcaaaaaaccctgctcgtatgatggagatgcagtggtcctggtggcaagggcagattcaaatttaccagaacgtgttaatgcgtagtgtagccaaggacgtagccccctttatccagccagagtccggagatcgtcgcttcaactcgccactttggcaagaacatccaaattttgatttactgagtcaatcctacttgttgttttctcagttggttcaaaatatggtggatgtcgttgaaggagtacctgataaggtccgctatcgcatccatttctttacacgtcagatgatcaatgcgttgtctccttctaatttcctgtggacgaaccctgaagtaattcaacagacggtcgctgaacagggtgagaatttagtacgcgggatgcaagtatttcacgatgatgtaatgaattcgggtaaatatttgagcatccgtatggtaaatagcgacagtttctctcttggcaaggacttggcgtatacgccaggagccgtagttttcgagaacgacatctttcagcttcttcaatacgaagccacaaccgagaacgtatatcaaacccctattcttgtcgtacctcccttcatcaacaagtactacgtgctggacctgcgcgaacagaatagcttggttaattggctgcgccaacaaggacatacggtgtttttgatgtcgtggcgtaaccccaacgcagagcagaaggagcttaccttcgctgacttaattacccaaggatcggtagaagcattacgtgttatcgaagaaatcacgggagagaaagaagctaactgtattggatattgcatcggtggtacacttctggctgctacccaggcatattatgtagctaaacgcctgaaaaatcacgtaaagtcagcgacttatatggcgacgattattgattttgagaaccccggctcattgggtgttttcattaatgagccggtcgtaagtggacttgaaaaccttaataatcaacttggttacttcgacgggcgtcaacttgcagtgacattttcgttgttgcgcgaaaacaccttgtattggaattattacatcgataattacttgaagggtaaggaaccgtccgactttgacatcttatactggaactcggatggtacgaatatcccagcaaagattcacaatttcctgttacgtaacctttatcttaacaacgaacttatttctccaaatgccgtcaaagttaatggtgtgggtttaaacctttcgcgcgtgaagactccatcattcttcattgctacgcaggaggaccatatcgcattgtgggatacctgttttcgcggcgcggattacctggggggtgagagcacacttgtgcttggggaaagcggacacgtcgccggcattgtcaacccgccttctcgtaacaagtatggttgttacacgaacgccgccaagtttgaaaataccaagcaatggcttgacggtgcagaatatcatcccgaaagctggtggttacgttggcaggcatgggtcacgccttatactggagagcaggttcctgcgcgtaatttgggaaacgcacagtaccccagtattgaagcggcccctgggcgttatgtgctggtaaacctgttttaacgctcacatacaagcaatctataattattcacggtataaatgaaagatgttgttatcgtagccgctaaacgcactgcgatcggttcctttctggggagtctggcttccctgagcgcccctcagttgggtcagacggctatccgcgcagttttggattctgcaaatgtgaaaccagaacaagtggaccaagtaattatggggaatgtgctgaccaccggcgttgggcaaaatcctgctcgtcaggcagcaatcgccgctgggattcctgtacaagttcccgccagcacgcttaatgtagtgtgtgggtccggattacgtgccgttcacctggcagctcaagccatccaatgcgatgaagccgatatcgtcgttgccggaggtcaagaatcaatgtcccagtctgctcattacatgcagcttcgcaatggccagaaaatgggtaacgcacagttagtcgattcaatggtggccgacggcttgaccgacgcgtataatcaataccagatgggtatcaccgcggagaatatcgtcgaaaaacttggtcttaatcgtgaagaacaagaccagcttgctctgacaagtcaacaacgtgctgcagcagcgcaggctgccggaaaattcaaggatgaaattgcggtcgtttcgattccccagcgcaaaggagagccggtcgtcttcgcggaagacgaatatatcaaggccaatacctcgttggaatccttgacgaaactgcgtccagcattcaaaaaagacggttctgttacagccggcaacgcatctggcattaatgatggggcagccgcggtcctgatgatgtccgccgacaaagcggctgaactgggcttaaagcctttagcacgcattaaaggttacgcgatgtcaggaattgagccggaaatcatgggactgggtcctgtagacgccgttaagaaaacccttaataaggctggttggtccttagaccaggtcgatctgatcgaggccaatgaggcttttgctgcccaagcactgggagtagccaaggagcttgggctggacctggacaaggtaaatgttaacggaggtgcgatcgcgctgggacacccgatcggggcttcgggttgtcgtatcttggtcacgttattacacgaaatgcagcgtcgtgatgcaaagaagggtatcgccacattgtgtgtgggaggtggaatgggggtggcgcttgccgttgagcgcgattaa

SEQ ID NO: 140 - protein, 503 aa, Artificial Sequence
  Description of Artificial Sequence Synthetic polypeptide
MNANLFARLFDKLDDPHKLAIETAAGDKISYAELVARAGRVANVLVARGLQVGDRVAAQTEKSVEALVLYLATVRAGGVYLPLNTAYTLHELDYFITDAEPKIVVCDPSKRDGIAAIAAKVGATVETLGPDGRGSLTDAAAGASEAFATIDRGADDLAAILYTSGTTGRSKGAMLSHDNLASNSLTLVDYWRFTPDDVLIHALPIYHTHGLFVASNVTLFARGSMIFLPKFDPDKILDLMARATVLMGVPTFYTRLLQSPRLTKETTGHMRLFISGSAPLLADTHREWSAKTGHAVLERYGMTETNMNTSNPYDGDRVPGAVGPALPGVSARVTDPETGKELPRGDIGMIEVKGPNVFKGYWRMPEKTKSEFRDDGFFITGDLGKIDERGYVHILGRGKDLVITGGFNVYPKEIESEIDAMPGVVESAVIGVPHADFGEGVTAVVVRDKGATIDEAQVLHGLDGQLAKFKMPKKVIFVDDLPRNTMGKVQKNVLRETYKDIYK

SEQ ID NO: 141 - DNA, 1509 bp, Artificial Sequence
  Description of Artificial Sequence Synthetic polynucleotide
atgaatgcaaatctgtttgctcgtctgttcgacaaattagacgatccacataagttagccattgaaactgctgcaggtgataagatttcgtatgcagagcttgttgcccgcgcaggtcgcgtcgcaaatgtacttgtagcccgcggactgcaggtaggagatcgtgtagctgctcagacagagaaatctgtagaagcgttggttttatatttagcaactgtgcgtgctgggggggtataccttccactgaacaccgcatatactttacatgaattagattacttcatcacagacgccgagccgaaaattgttgtctgcgatccatcgaagcgcgacgggatcgctgccattgcagcaaaggtaggcgcgacagtcgaaactcttggaccggatggccgtggctctcttactgacgccgctgcgggagcctcagaagcctttgcaactattgatcgcggcgccgacgatctggcggctatcctttataccagcgggaccacggggcgtagcaagggtgcgatgctttcgcacgacaatctggcaagcaactcgcttacactggtggattactggcgcttcacaccggacgacgtgttgattcatgcattgccaatttaccacacgcacggattatttgtcgcatccaatgtgactttattcgcgcgcgggtcgatgattttcttacccaaattcgatccggataagattttagaccttatggctcgtgcaacggttttaatgggcgtaccgactttctacactcgcctgcttcagagcccgcgcttgacgaaggagacaacgggtcacatgcgcttattcattagcggcagtgcccccctgttggcagacactcaccgtgaatggtccgctaaaaccggacacgcagttttagaacgttatgggatgacggagacaaacatgaacacgagcaatccatatgatggtgaccgtgtaccgggggccgtcggtcccgcattaccaggggtatctgctcgcgtcactgatccggaaactggaaaagagctgccgcgtggtgacatcggaatgattgaagttaaaggacccaacgtattcaaaggatattggcgtatgccggaaaagactaagtcggagtttcgcgacgatggtttcttcattacaggagatttggggaaaatcgatgaacgtgggtatgttcacattcttgggcgcggtaaggatcttgtgatcaccggtggctttaacgtctatccaaaagaaattgaatcagagatcgacgccatgccaggggtagtggaatctgcggtaattggcgtgccccatgcggattttggtgaaggcgtcaccgccgtcgttgtacgcgataaaggagccacgatcgatgaagcccaggtacttcatggactggacggacagttagccaagtttaagatgccgaagaaggtaatctttgtggacgatcttcctcgtaacacaatgggtaaggtacaaaaaaacgttctgcgcgagacttacaaagacatttataaa

SEQ ID NO: 142 - DNA, 305 bp, Artificial Sequence
  Description of Artificial Sequence Synthetic polynucleotide
cagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacctctagaaataattttgtttaactttaagaaggagatatacat

SEQ ID NO: 143 - DNA, 897 bp, Artificial Sequence
  Description of Artificial Sequence Synthetic polynucleotide
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcat

SEQ ID NO: 144 - protein, 298 aa, Artificial Sequence
  Description of Artificial Sequence Synthetic polypeptide
MQYGQLVSSLNGGSMKSMAEAQNDPLLPGYSFNAHLVAGLTPIEANGYLDFFIDRPLGMKGYILNLTIRGQGVVKNQGREFVCRPGDILLFPPGEIHHYGRHPEAHEWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCE

SEQ ID NO: 145 - DNA, 280 bp, Artificial Sequence
  Description of Artificial Sequence Synthetic polynucleotide
cggtgagcatcacatcaccacaattcagcaaattgtgaacatcatcacgttcatctttccctggttgccaatggcccattttcctgtcagtaacgagaaggtcgcgaatcaggcgctttttagactggtcgtaatgaaattcagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 146 - DNA, 326 bp, Artificial Sequence
  Description of Artificial Sequence Synthetic polynucleotide
attcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 147 - DNA, 22 bp, Artificial Sequence
  Description of Artificial Sequence Synthetic oligonucleotide
ggaattgtgagcgctcacaatt

SEQ ID NO: 148 - DNA, 1083 bp, Artificial Sequence
  Description of Artificial Sequence Synthetic polynucleotide
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcat

SEQ ID NO: 149 - protein, 360 aa, Artificial Sequence
  Description of Artificial Sequence Synthetic polypeptide
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 150 - DNA, 222 bp, Artificial Sequence
  Description of Artificial Sequence Synthetic polynucleotide
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 151 - DNA, 714 bp, Artificial Sequence
  Description of Artificial Sequence Synthetic polynucleotide
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 152 - DNA, 30 bp, Artificial Sequence
  Description of Artificial Sequence Synthetic oligonucleotide
gtttatacataggcgagtactctgttatgg

SEQ ID NO: 153 - DNA, 30 bp, Artificial Sequence
  Description of Artificial Sequence Synthetic oligonucleotide
agaggttccaactttcaccataatgaaaca

SEQ ID NO: 154 - DNA, 30 bp, Artificial Sequence
  Description of Artificial Sequence Synthetic oligonucleotide
taaacaactaacggacaattctacctaaca

SEQ ID NO: 155 - DNA, 30 bp, Artificial Sequence
  Description of Artificial Sequence Synthetic oligonucleotide
acatcaagccaaattaaacaggattaacac

SEQ ID NO: 156 - DNA, 30 bp, Artificial Sequence
  Description of Artificial Sequence Synthetic oligonucleotide
gaggtaaaatagtcaacacgcacggtgtta

SEQ ID NO: 157 - DNA, 30 bp, Artificial Sequence
  Description of Artificial Sequence Synthetic oligonucleotide
caggccggaataactccctataatgcgcca

SEQ ID NO: 158 - DNA, 30 bp, Artificial Sequence
  Description of Artificial Sequence Synthetic oligonucleotide
ggctagctcagtcctaggtacagtgctagc

SEQ ID NO: 159 - DNA, 30 bp, Artificial Sequence
  Description of Artificial Sequence Synthetic oligonucleotide
agctagctcagtcctaggtattatgctagc

SEQ ID NO: 160 - DNA, 30 bp, Artificial Sequence
  Description of Artificial Sequence Synthetic oligonucleotide
agctagctcagtcctaggtactgtgctagc

SEQ ID NO: 161 - DNA, 30 bp, Artificial Sequence
  Description of Artificial Sequence Synthetic oligonucleotide
agctagctcagtcctagggattatgctagc

SEQ ID NO: 162 - DNA, 30 bp, Artificial Sequence
  Description of Artificial Sequence Synthetic oligonucleotide
agctagctcagtcctaggtattgtgctagc

SEQ ID NO: 163 - DNA, 30 bp, Artificial Sequence
  Description of Artificial Sequence Synthetic oligonucleotide
ggctagctcagtcctaggtactatgctagc

SEQ ID NO: 164 - DNA, 30 bp, Artificial Sequence
  Description of Artificial Sequence Synthetic oligonucleotide
ggctagctcagtcctaggtatagtgctagc

SEQ ID NO: 165 - DNA, 30 bp, Artificial Sequence
  Description of Artificial Sequence Synthetic oligonucleotide
ggctagctcagccctaggtattatgctagc

SEQ ID NO: 166 - DNA, 30 bp, Artificial Sequence
  Description of Artificial Sequence Synthetic oligonucleotide
agctagctcagtcctaggtataatgctagc

SEQ ID NO: 167 - DNA, 30 bp, Artificial Sequence
  Description of Artificial Sequence Synthetic oligonucleotide
agctagctcagtcctagggactgtgctagc

SEQ ID NO: 168 - DNA, 30 bp, Artificial Sequence
  Description of Artificial Sequence Synthetic oligonucleotide
ggctagctcagtcctaggtacaatgctagc

SEQ ID NO: 169 - DNA, 30 bp, Artificial Sequence; identical to SEQ ID NO: 164
  Description of Artificial Sequence Synthetic oligonucleotide

SEQ ID NO: 170 - DNA, 30 bp, Artificial Sequence; identical to SEQ ID NO: 161
  Description of Artificial Sequence Synthetic oligonucleotide

SEQ ID NO: 171 - DNA, 30 bp, Artificial Sequence
  Description of Artificial Sequence Synthetic oligonucleotide
ggctagctcagtcctagggattatgctagc

SEQ ID NO: 172 - DNA, 30 bp, Artificial Sequence; identical to SEQ ID NO: 168
  Description of Artificial Sequence Synthetic oligonucleotide

SEQ ID NO: 173 - DNA, 30 bp, Artificial Sequence
  Description of Artificial Sequence Synthetic oligonucleotide
agctagctcagcccttggtacaatgctagc

SEQ ID NO: 174 - DNA, 30 bp, Artificial Sequence
  Description of Artificial Sequence Synthetic oligonucleotide
agctagctcagtcctagggactatgctagc

SEQ ID NO: 175 - DNA, 30 bp, Artificial Sequence
  Description of Artificial Sequence Synthetic oligonucleotide
agctagctcagtcctagggattgtgctagc

SEQ ID NO: 176 - DNA, 30 bp, Artificial Sequence
  Description of Artificial Sequence Synthetic oligonucleotide
ggctagctcagtcctaggtattgtgctagc

SEQ ID NO: 177 - DNA, 30 bp, Artificial Sequence; identical to SEQ ID NO: 166
  Description of Artificial Sequence Synthetic oligonucleotide

SEQ ID NO: 178 - DNA, 30 bp, Artificial Sequence
  Description of Artificial Sequence Synthetic oligonucleotide
ggctagctcagtcctaggtattatgctagc

SEQ ID NO: 179 - DNA, 30 bp, Artificial Sequence; identical to SEQ ID NO: 168
  Description of Artificial Sequence Synthetic oligonucleotide

SEQ ID NO: 180 - DNA, 30 bp, Artificial Sequence
  Description of Artificial Sequence Synthetic oligonucleotide
aaagtgtgacgccgtgcaaataatcaatgt

SEQ ID NO: 181 - DNA, 30 bp, Artificial Sequence
  Description of Artificial Sequence Synthetic oligonucleotide
gacgaatacttaaaatcgtcatacttattt

SEQ ID NO: 182 - DNA, 30 bp, Artificial Sequence
  Description of Artificial Sequence Synthetic oligonucleotide
aaacctttcgcggtatggcatgatagcgcc

SEQ ID NO: 183 - DNA, 30 bp, Artificial Sequence
  Description of Artificial Sequence Synthetic oligonucleotide
tgatagcgcccggaagagagtcaattcagg

SEQ ID NO: 184 - DNA, 30 bp, Artificial Sequence
  Description of Artificial Sequence Synthetic oligonucleotide
ttatttaccgtgacgaactaattgctcgtg

SEQ ID NO: 185 - DNA, 30 bp, Artificial Sequence
  Description of Artificial Sequence Synthetic oligonucleotide
catacgccgttatacgttgtttacgctttg

SEQ ID NO: 186 - DNA, 30 bp, Artificial Sequence
  Description of Artificial Sequence Synthetic oligonucleotide
ttatgcttccggctcgtatgttgtgtggac

SEQ ID NO: 187 - DNA, 30 bp, Artificial Sequence
  Description of Artificial Sequence Synthetic oligonucleotide
ttatgcttccggctcgtatggtgtgtggac

SEQ ID NO: 188 - DNA, 30 bp, Artificial Sequence; identical to SEQ ID NO: 163
  Description of Artificial Sequence Synthetic oligonucleotide

SEQ ID NO: 189 - DNA, 30 bp, Artificial Sequence
  Description of Artificial Sequence Synthetic oligonucleotide
atatatatatatatataatggaagcgtttt

SEQ ID NO: 190 - DNA, 30 bp, Artificial Sequence; identical to SEQ ID NO: 189
  Description of Artificial Sequence Synthetic oligonucleotide

SEQ ID NO: 191 - DNA, 30 bp, Artificial Sequence
  Description of Artificial Sequence Synthetic oligonucleotide
ccccgaaagcttaagaatataattgtaagc

SEQ ID NO: 192 - DNA, 30 bp, Artificial Sequence; identical to SEQ ID NO: 191
  Description of Artificial Sequence Synthetic oligonucleotide

SEQ ID NO: 193 - DNA, 30 bp, Artificial Sequence
  Description of Artificial Sequence Synthetic oligonucleotide
tgacaatatatatatatatataatgctagc

SEQ ID NO: 194 - DNA, 30 bp, Artificial Sequence
  Description of Artificial Sequence Synthetic oligonucleotide
acaatatatatatatatatataatgctagc

SEQ ID NO: 195 - DNA, 30 bp, Artificial Sequence
  Description of Artificial Sequence Synthetic oligonucleotide
aatatatatatatatatatataatgctagc

SEQ ID NO: 196 - DNA, 30 bp, Artificial Sequence
  Description of Artificial Sequence Synthetic oligonucleotide
tatatatatatatatatatataatgctagc

SEQ ID NO: 197 - DNA, 30 bp, Artificial Sequence; identical to SEQ ID NO: 196
  Description of Artificial Sequence Synthetic oligonucleotide

SEQ ID NO: 198 - DNA, 30 bp, Artificial Sequence
  Description of Artificial Sequence Synthetic oligonucleotide
aaaaaaaaaaaaaaaaaatataatgctagc

SEQ ID NO: 199 - DNA, 30 bp, Artificial Sequence; identical to SEQ ID NO: 198
  Description of Artificial Sequence Synthetic oligonucleotide

SEQ ID NO: 200 - DNA, 30 bp, Artificial Sequence
  Description of Artificial Sequence Synthetic oligonucleotide
ggaattgtgagcggataacaatttcacaca

SEQ ID NO: 201 - DNA, 30 bp, Artificial Sequence; identical to SEQ ID NO: 200
  Description of Artificial Sequence Synthetic oligonucleotide

SEQ ID NO: 202 - DNA, 30 bp, Artificial Sequence; identical to SEQ ID NO: 200
  Description of Artificial Sequence Synthetic oligonucleotide

SEQ ID NO: 203 - DNA, 30 bp, Artificial Sequence; identical to SEQ ID NO: 200
  Description of Artificial Sequence Synthetic oligonucleotide

SEQ ID NO: 204 - DNA, 30 bp, Artificial Sequence; identical to SEQ ID NO: 200
  Description of Artificial Sequence Synthetic oligonucleotide

SEQ ID NO: 205 - DNA, 30 bp, Artificial Sequence; identical to SEQ ID NO: 200
  Description of Artificial Sequence Synthetic oligonucleotide

SEQ ID NO: 206 - DNA, 30 bp, Artificial Sequence; identical to SEQ ID NO: 200
  Description of Artificial Sequence Synthetic oligonucleotide

SEQ ID NO: 207 - DNA, 30 bp, Artificial Sequence; identical to SEQ ID NO: 200
  Description of Artificial Sequence Synthetic oligonucleotide

SEQ ID NO: 208 - DNA, 30 bp, Artificial Sequence; identical to SEQ ID NO: 200
  Description of Artificial Sequence Synthetic oligonucleotide

SEQ ID NO: 209 - DNA, 30 bp, Artificial Sequence; identical to SEQ ID NO: 200
  Description of Artificial Sequence Synthetic oligonucleotide

SEQ ID NO: 210 - DNA, 30 bp, Artificial Sequence; identical to SEQ ID NO: 200
  Description of Artificial Sequence Synthetic oligonucleotide

SEQ ID NO: 211 - DNA, 30 bp, Artificial Sequence; identical to SEQ ID NO: 200
  Description of Artificial Sequence Synthetic oligonucleotide

SEQ ID NO: 212 - DNA, 30 bp, Artificial Sequence; identical to SEQ ID NO: 200
  Description of Artificial Sequence Synthetic oligonucleotide

SEQ ID NO: 213 - DNA, 30 bp, Artificial Sequence; identical to SEQ ID NO: 200
  Description of Artificial Sequence Synthetic oligonucleotide

SEQ ID NO: 214 - DNA, 30 bp, Artificial Sequence
  Description of Artificial Sequence Synthetic oligonucleotide
gattaaagaggagaaatactagagtactag

SEQ ID NO: 215 - DNA, 30 bp, Artificial Sequence
  Description of Artificial Sequence Synthetic oligonucleotide
caccttcgggtgggcctttctgcgtttata

SEQ ID NO: 216 - DNA, 30 bp, Artificial Sequence; identical to SEQ ID NO: 215
  Description of Artificial Sequence Synthetic oligonucleotide

SEQ ID NO: 217 - DNA, 30 bp, Artificial Sequence; identical to SEQ ID NO: 215
  Description of Artificial Sequence Synthetic oligonucleotide

SEQ ID NO: 218 - DNA, 30 bp, Artificial Sequence; identical to SEQ ID NO: 215
  Description of Artificial Sequence Synthetic oligonucleotide

SEQ ID NO: 219 - DNA, 30 bp, Artificial Sequence; identical to SEQ ID NO: 158
  Description of Artificial Sequence Synthetic oligonucleotide

SEQ ID NO: 220 - DNA, 30 bp, Artificial Sequence
  Description of Artificial Sequence Synthetic oligonucleotide
tgctagctactagagattaaagaggagaaa

SEQ ID NO: 221 - DNA, 30 bp, Artificial Sequence
  Description of Artificial Sequence Synthetic oligonucleotide
ttgtgagcggataacaagatactgagcaca

SEQ ID NO: 222 - DNA, 30 bp, Artificial Sequence; identical to SEQ ID NO: 221
  Description of Artificial Sequence Synthetic oligonucleotide

SEQ ID NO: 223 - DNA, 30 bp, Artificial Sequence; identical to SEQ ID NO: 221
  Description of Artificial Sequence Synthetic oligonucleotide

SEQ ID NO: 224 - DNA, 30 bp, Artificial Sequence; identical to SEQ ID NO: 158
  Description of Artificial Sequence Synthetic oligonucleotide

SEQ ID NO: 225 - DNA, 30 bp, Artificial Sequence; identical to SEQ ID NO: 159
  Description of Artificial Sequence Synthetic oligonucleotide

SEQ ID NO: 226 - DNA, 30 bp, Artificial Sequence; identical to SEQ ID NO: 160
  Description of Artificial Sequence Synthetic oligonucleotide

SEQ ID NO: 227 - DNA, 30 bp, Artificial Sequence; identical to SEQ ID NO: 161
  Description of Artificial Sequence Synthetic oligonucleotide

SEQ ID NO: 228 - DNA, 30 bp, Artificial Sequence; identical to SEQ ID NO: 164
  Description of Artificial Sequence Synthetic oligonucleotide

SEQ ID NO: 229 - DNA, 30 bp, Artificial Sequence; identical to SEQ ID NO: 171
  Description of Artificial Sequence Synthetic oligonucleotide

SEQ ID NO: 230 - DNA, 30 bp, Artificial Sequence; identical to SEQ ID NO: 168
  Description of Artificial Sequence Synthetic oligonucleotide

SEQ ID NO: 231 - DNA, 30 bp, Artificial Sequence; identical to SEQ ID NO: 175
  Description of Artificial Sequence Synthetic oligonucleotide

SEQ ID NO: 232 - DNA, 30 bp, Artificial Sequence; identical to SEQ ID NO: 176
  Description of Artificial Sequence Synthetic oligonucleotide

SEQ ID NO: 233 - DNA, 30 bp, Artificial Sequence
  Description of Artificial Sequence Synthetic oligonucleotide
cctgtttttatgttattctctctgtaaagg

SEQ ID NO: 234 - DNA, 30 bp, Artificial Sequence
  Description of Artificial Sequence Synthetic oligonucleotide
aaatatttgcttatacaatcttcctgtttt

SEQ ID NO: 235 - DNA, 30 bp, Artificial Sequence
  Description of Artificial Sequence Synthetic oligonucleotide
gctgataaaccgatacaattaaaggctcct

SEQ ID NO: 236 - DNA, 30 bp, Artificial Sequence
  Description of Artificial Sequence Synthetic oligonucleotide
ctcttctcagcgtcttaatctaagctatcg

SEQ ID NO: 237 - DNA, 30 bp, Artificial Sequence
  Description of Artificial Sequence Synthetic oligonucleotide
atgagccagttcttaaaatcgcataaggta

SEQ ID NO: 238 - DNA, 30 bp, Artificial Sequence
  Description of Artificial Sequence Synthetic oligonucleotide
ctattgattgtgacaaaataaacttattcc

SEQ ID NO: 239 - DNA, 30 bp, Artificial Sequence
  Description of Artificial Sequence Synthetic oligonucleotide
gtttcgcgcttggtataatcgctgggggtc

SEQ ID NO: 240 - DNA, 30 bp, Artificial Sequence
  Description of Artificial Sequence Synthetic oligonucleotide
ctttgcttctgactataatagtcagggtaa

SEQ ID NO: 241 - DNA, 30 bp, Artificial Sequence
  Description of Artificial Sequence Synthetic oligonucleotide
aaaccgatacaattaaaggctcctgctagc

SEQ ID NO: 242 - DNA, 30 bp, Artificial Sequence
  Description of Artificial Sequence Synthetic oligonucleotide
caccacactgatagtgctagtgtagatcac

SEQ ID NO: 243 - DNA, 30 bp, Artificial Sequence
  Description of Artificial Sequence Synthetic oligonucleotide
gccggaataactccctataatgcgccacca

SEQ ID NO: 244 - DNA, 30 bp, Artificial Sequence
  Description of Artificial Sequence Synthetic oligonucleotide
ttgacaagcttttcctcagctccgtaaact

SEQ ID NO: 245 - DNA, 30 bp, Artificial Sequence
  Description of Artificial Sequence Synthetic oligonucleotide
ggtttcaaaattgtgatctatatttaacaa

SEQ ID NO: 246 - DNA, 30 bp, Artificial Sequence; identical to SEQ ID NO: 245
  Description of Artificial Sequence Synthetic oligonucleotide

SEQ ID NO: 247 - DNA, 30 bp, Artificial Sequence
  Description of Artificial Sequence Synthetic oligonucleotide
tctattccaataaagaaatcttcctgcgtg

SEQ ID NO: 248 - DNA, 30 bp, Artificial Sequence
  Description of Artificial Sequence Synthetic oligonucleotide
gaccgaatatatagtggaaacgtttagatg

SEQ ID NO: 249 - DNA, 30 bp, Artificial Sequence
  Description of Artificial Sequence Synthetic oligonucleotide
ccacatcctgtttttaaccttaaaatggca

SEQ ID NO: 250 - DNA, 30 bp, Artificial Sequence
  Description of Artificial Sequence Synthetic oligonucleotide
aaaaatgggctcgtgttgtacaataaatgt

SEQ ID NO: 251 - DNA, 30 bp, Artificial Sequence
  Description of Artificial Sequence Synthetic oligonucleotide
aaaaaaagcgcgcgattatgtaaaatataa

SEQ ID NO: 252 - DNA, 30 bp, Artificial Sequence
  Description of Artificial Sequence Synthetic oligonucleotide
aattgcagtaggcatgacaaaatggactca

SEQ ID NO: 253 - DNA, 30 bp, Artificial Sequence
  Description of Artificial Sequence Synthetic oligonucleotide
caagcttttcctttataatagaatgaatga

SEQ ID NO: 254 - DNA, 30 bp, Artificial Sequence
  Description of Artificial Sequence Synthetic oligonucleotide
tctaagctagtgtattttgcgtttaatagt

SEQ ID NO: 255 - DNA, 30 bp, Artificial Sequence
  Description of Artificial Sequence Synthetic oligonucleotide
aatgggctcgtgttgtacaataaatgtagt

SEQ ID NO: 256 - DNA, 30 bp, Artificial Sequence
  Description of Artificial Sequence Synthetic oligonucleotide
atccttatcgttatgggtattgtttgtaat

SEQ ID NO: 257 - DNA, 30 bp, Artificial Sequence
  Description of Artificial Sequence Synthetic oligonucleotide
taaaagaattgtgagcgggaatacaacaac

SEQ ID NO: 258 - DNA, 30 bp, Artificial Sequence; identical to SEQ ID NO: 251
  Description of Artificial Sequence Synthetic oligonucleotide

SEQ ID NO: 259 - DNA, 30 bp, Artificial Sequence
  Description of Artificial Sequence Synthetic oligonucleotide
tacaaaataattcccctgcaaacattatca

SEQ ID NO: 260 - DNA, 30 bp, Artificial Sequence
  Description of Artificial Sequence Synthetic oligonucleotide
tacaaaataattcccctgcaaacattatcg

SEQ ID NO: 261 - DNA, 30 bp, Artificial Sequence
  Description of Artificial Sequence Synthetic oligonucleotide
agggaatacaagctacttgttctttttgca

SEQ ID NO: 262 - DNA, 23 bp, Artificial Sequence
  Description of Artificial Sequence Synthetic oligonucleotide
taatacgactcactatagggaga

SEQ ID NO: 263 - DNA, 28 bp, Artificial Sequence
  Description of Artificial Sequence Synthetic oligonucleotide
gaatttaatacgactcactatagggaga

SEQ ID NO: 264 - DNA, 19 bp, Artificial Sequence
  Description of Artificial Sequence Synthetic oligonucleotide
taatacgactcactatagg

SEQ ID NO: 265 - DNA, 30 bp, Artificial Sequence
  Description of Artificial Sequence Synthetic oligonucleotide
gagtcgtattaatacgactcactatagggg

SEQ ID NO: 266 - DNA, 30 bp, Artificial Sequence
  Description of Artificial Sequence Synthetic oligonucleotide
agtgagtcgtactacgactcactatagggg

SEQ ID NO: 267 - DNA, 30 bp, Artificial Sequence
  Description of Artificial Sequence Synthetic oligonucleotide
gagtcgtattaatacgactctctatagggg

SEQ ID NO: 268 - DNA, 18 bp, Artificial Sequence
  Description of Artificial Sequence Synthetic oligonucleotide
taatacgactcactatag

SEQ ID NO: 269 - DNA, 23 bp, Artificial Sequence
  Description of Artificial Sequence Synthetic oligonucleotide
taatacgactcactatagggaga

SEQ ID NO: 270 - DNA, 23 bp, Artificial Sequence
  Description of Artificial Sequence Synthetic oligonucleotide
ttatacgactcactatagggaga

SEQ ID NO: 271 - DNA, 23 bp, Artificial Sequence
  Description of Artificial Sequence Synthetic oligonucleotide
gaatacgactcactatagggaga

SEQ ID NO: 272 - DNA, 23 bp, Artificial Sequence
  Description of Artificial Sequence Synthetic oligonucleotide
taatacgtctcactatagggaga

SEQ ID NO: 273 - DNA, 23 bp, Artificial Sequence
  Description of Artificial Sequence Synthetic oligonucleotide
tcatacgactcactatagggaga

SEQ ID NO: 274 - DNA, 30 bp, Artificial Sequence
  Description of Artificial Sequence Synthetic oligonucleotide
taatacgactcactatagggagaccacaac

SEQ ID NO: 275 - DNA, 30 bp, Artificial Sequence
  Description of Artificial Sequence Synthetic oligonucleotide
taattgaactcactaaagggagaccacagc

SEQ ID NO: 276 - DNA, 30 bp, Artificial Sequence
  Description of Artificial Sequence Synthetic oligonucleotide
cgaagtaatacgactcactattagggaaga

SEQ ID NO: 277 - DNA, 19 bp, Artificial Sequence
  Description of Artificial Sequence Synthetic oligonucleotide
atttaggtgacactataga

SEQ ID NO: 278 - DNA, 30 bp, Artificial Sequence
  Description of Artificial Sequence Synthetic oligonucleotide
acaaacacaaatacacacactaaattaata

SEQ ID NO: 279 - DNA, 30 bp, Artificial Sequence
  Description of Artificial Sequence Synthetic oligonucleotide
ccaagcatacaatcaactatctcatataca

SEQ ID NO: 280 - DNA, 30 bp, Artificial Sequence
  Description of Artificial Sequence Synthetic oligonucleotide
gatacaggatacagcggaaacaacttttaa

SEQ ID NO: 281 - DNA, 30 bp, Artificial Sequence
  Description of Artificial Sequence Synthetic oligonucleotide
tttcaagctataccaagcatacaatcaact

SEQ ID NO: 282 - DNA, 30 bp, Artificial Sequence
  Description of Artificial Sequence Synthetic oligonucleotide
cctttgcagcataaattactatacttctat

SEQ ID NO: 283 - DNA, 30 bp, Artificial Sequence; identical to SEQ ID NO: 282
  Description of Artificial Sequence Synthetic oligonucleotide

SEQ ID NO: 284 - DNA, 30 bp, Artificial Sequence; identical to SEQ ID NO: 282
  Description of Artificial Sequence Synthetic oligonucleotide

SEQ ID NO: 285 - DNA, 30 bp, Artificial Sequence; identical to SEQ ID NO: 282
  Description of Artificial Sequence Synthetic oligonucleotide

SEQ ID NO: 286 - DNA, 30 bp, Artificial Sequence; identical to SEQ ID NO: 282
  Description of Artificial Sequence Synthetic oligonucleotide

SEQ ID NO: 287 - DNA, 30 bp, Artificial Sequence
  Description of Artificial Sequence Synthetic oligonucleotide
ttatctactttttacaacaaatataaaaca

SEQ ID NO: 288 - DNA, 30 bp, Artificial Sequence; identical to SEQ ID NO: 278
  Description of Artificial Sequence Synthetic oligonucleotide

SEQ ID NO: 289 - DNA, 30 bp, Artificial Sequence
  Description of Artificial Sequence Synthetic oligonucleotide
gtttcgaataaacacacataaacaaacaaa

SEQ ID NO: 290 - DNA, 30 bp, Artificial Sequence; identical to SEQ ID NO: 279
  Description of Artificial Sequence Synthetic oligonucleotide

SEQ ID NO: 291 - DNA, 30 bp, Artificial Sequence
  Description of Artificial Sequence Synthetic oligonucleotide
accatcaaaggaagctttaatcttctcata

SEQ ID NO: 292 - DNA, 30 bp, Artificial Sequence
  Description of Artificial Sequence Synthetic oligonucleotide
agaacccactgcttactggcttatcgaaat

SEQ ID NO: 293 - DNA, 30 bp, Artificial Sequence
  Description of Artificial Sequence Synthetic oligonucleotide
ggccgtttttggcttttttgttagacgaag

SEQ ID NO: 294 - DNA, 66 bp, Artificial Sequence
  Description of Artificial Sequence Synthetic oligonucleotide
ataagtgccttcccatcaaaaaaatattctcaacataaaaaactttgtgtaatacttgtaacgcta

SEQ ID NO: 295 - DNA, 52 bp, Artificial Sequence
  Description of Artificial Sequence Synthetic oligonucleotide
aaaaagagtattgacttcgcatctttttgtacctataatagattcattgcta

SEQ ID NO: 296 - DNA, 59 bp, Artificial Sequence
  Description of Artificial Sequence Synthetic oligonucleotide
ggaaaatttttttaaaaaaaaaactttacagctagctcagtcctaggtattatgctagc

SEQ ID NO: 297 - DNA, 59 bp, Artificial Sequence
  Description of Artificial Sequence Synthetic oligonucleotide
ggaaaatttttttaaaaaaaaaactttacggctagctcagccctaggtattatgctagc

SEQ ID NO: 298 - DNA, 64 bp, Artificial Sequence
  Description of Artificial Sequence Synthetic oligonucleotide
ggaaaatttttttaaaaaaaaaacttgacagctagctcagtccttggtataatgctagcacgaa

SEQ ID NO: 299 - protein, 21 aa, Artificial Sequence
  Description of Artificial Sequence Synthetic peptide
MKQSTIALALLPLLFTPVTKA

SEQ ID NO: 300 - protein, 20 aa, Artificial Sequence
  Description of Artificial Sequence Synthetic peptide
KQSTIALALLPLLFTPVTKA

SEQ ID NO: 301 - protein, 22 aa, Artificial Sequence
  Description of Artificial Sequence Synthetic peptide
MMKRNILAVIVPALLVAGTANA

SEQ ID NO: 302 - protein, 15 aa, Artificial Sequence
  Description of Artificial Sequence Synthetic peptide
MRTLTLNELDSVSGG

SEQ ID NO: 303 - protein, 43 aa, Artificial Sequence
  Description of Artificial Sequence Synthetic polypeptide
MNNNDLFQASRRRFLAQLGGLTVAGMLGTSLLTPRRATAAQAA

SEQ ID NO: 304 - protein, 33 aa, Artificial Sequence
  Description of Artificial Sequence Synthetic polypeptide
MDVSRRQFFKICAGGMAGTTVAALGFAPKQALA

SEQ ID NO: 305 - protein, 45 aa, Artificial Sequence
  Description of Artificial Sequence Synthetic polypeptide
MKTKIPDAVLAAEVSRRGLVKTTAIGGLAMASSALTLPFSRIAHA

SEQ ID NO: 306 - protein, 21 aa, Artificial Sequence
  Description of Artificial Sequence Synthetic peptide
KYLLPTAAAGLLLLAAQPAMA

SEQ ID NO: 307 - protein, 52 aa, Artificial Sequence
  Description of Artificial Sequence Synthetic polypeptide
LNPLINEISKIISAAGNFDVKEERAAASLLQLSGNASDFSYGRNSITLTASA

SEQ ID NO: 308 - protein, 159 aa, Artificial Sequence
  Description of Artificial Sequence Synthetic polypeptide
CTTAATCCATTAATTAATGAAATCAGCAAAATCATTTCAGCTGCAGGTAATTTTGATGTTAAAGAGGAAAGAGCTGCAGCTTCTTTATTGCAGTTGTCCGGTAATGCCAGTGATTTTTCATATGGACGGAACTCAATAACTTTGACAGCATCAGCATAA

SEQ ID NO: 309 - protein, 607 aa, Artificial Sequence
  Description of Artificial Sequence Synthetic polypeptide
MVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAV

SEQ ID NO: 310 - protein, 148 aa, Artificial Sequence
  Description of Artificial Sequence Synthetic polypeptide
KATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPG

SEQ ID NO: 311 - protein, 658 aa, Artificial Sequence
  Description of Artificial Sequence Synthetic polypeptide
ITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYYRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 312 - DNA, 18 bp, Artificial Sequence
  Description of Artificial Sequence Synthetic consensus oligonucleotide
ttgttgayryrtcaacwa

SEQ ID NO: 313 - DNA, 15 bp, Artificial Sequence
  Description of Artificial Sequence Synthetic consensus oligonucleotide
ttataatnattataa

SEQ ID NO: 314 - DNA, 24 bp, Artificial Sequence
  Description of Artificial Sequence Synthetic oligonucleotide
ggaaaatttttttaaaaaaaaaac

SEQ ID NO: 315 - protein, 8 aa, Artificial Sequence
  Description of Artificial Sequence Synthetic consensus peptide
AGYGSTLT

SEQ ID NO: 316 - DNA, 12 bp, Artificial Sequence
  Description of Artificial Sequence Synthetic oligonucleotide
cggctcatctgg

SEQ ID NO: 317 - DNA, 14 bp, Artificial Sequence
  Description of Artificial Sequence Synthetic oligonucleotide
ggtttagccctaaa

SEQ ID NO: 318 - DNA, 43 bp, Artificial Sequence
  Description of Artificial Sequence Synthetic oligonucleotide
ctctagaaataattttgtttaactttaagaaggagatatacat

SEQ ID NO: 319 - protein, 237 aa, Artificial Sequence
  Description of Artificial Sequence Synthetic polypeptide
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 320 - DNA, 748 bp, Artificial Sequence
  Description of Artificial Sequence Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacat

SEQ ID NO: 321 - DNA, 225 bp, Artificial Sequence
  Description of Artificial Sequence Synthetic polynucleotide
tcacctttcccggattaaacgcttttttgcccggtggcatggtgctaccggcgatcacaaacggttaattatgacacaaattgacctgaatgaatatacagtattggaatgcattacccggagtgttgtgtaacaatgtctggccaggtttgtttcccggaaccgaggtcacaacatagtaaaagcgctattggtaatggtacaatcgcgcgtttacacttattc

SEQ ID NO: 322 - DNA, 1420 bp, Artificial Sequence
  Description of Artificial Sequence Synthetic polynucleotide
ttattatcgcaccgcaatcgggattttcgattcataaagcaggtcgtaggtcggcttgttgagcaggtcttgcagcgtgaaaccgtccagatacgtgaaaaacgacttcattgcaccgccgagtatgcccgtcagccggcaggacggcgtaatcaggcattcgttgttcgggcccatacactcgaccagctgcatcggttcgaggtggcggacgaccgcgccgatattgatgcgttcgggcggcgcggccagcctcagcccgccgcctttcccgcgtacgctgtgcaagaacccgcctttgaccagcgcggtaaccactttcatcaaatggcttttggaaatgccgtaggtcgaggcgatggtggcgatattgaccagcgcgtcgtcgttgacggcggtgtagatgaggacgcgcagcccgtagtcggtatgttgggtcagatacatacaacctccttagtacatgcaaaattatttctagagcaacatacgagccggaagcataaagtgtaaagcctggggtgcctaatgagttgagttgaggaattataacaggaagaaatattcctcatacgcttgtaattcctctatggttgttgacaattaatcatcggctcgtataatgtataacattcatattttgtgaattttaaactctagaaataattttgtttaactttaagaaggagatatacatatggctagcaaaggcgaagaattgttcacgggcgttgttcctattttggttgaattggatggcgatgttaatggccataaattcagcgttagcggcgaaggcgaaggcgatgctacgtatggcaaattgacgttgaaattcatttgtacgacgggcaaattgcctgttccttggcctacgttggttacgacgttcagctatggcgttcaatgtttcagccgttatcctgatcatatgaaacgtcatgatttcttcaaaagcgctatgcctgaaggctatgttcaagaacgtacgattagcttcaaagatgatggcaattataaaacgcgtgctgaagttaaattcgaaggcgatacgttggttaatcgtattgaattgaaaggcattgatttcaaagaagatggcaatattttgggccataaattggaatataattataatagccataatgtttatattacggctgataaacaaaaaaatggcattaaagctaatttcaaaattcgtcataatattgaagatggcagcgttcaattggctgatcattatcaacaaaatacgcctattggcgatggccctgttttgttgcctgataatcattatttgagcacgcaaagcgctttgagcaaagatcctaatgaaaaacgtgatcatatggttttgttggaattcgttacggctgctggcattacgcatggcatggatgaattgtataaataataa

(19) United States
(12) Patent Application Publication
(10) Pub. No.: US 2017/0216370 A1
(43) Pub. Date: Aug. 3, 2017

(54) BACTERIA ENGINEERED TO TREAT DISORDERS INVOLVING PROPIONATE CATABOLISM

(71) Applicant: Synlogic, Inc., Cambridge, MA (US)

(72) Inventors: Dean Falb, Sherborn, MA (US); Paul F. Miller, Salem, CT (US); Alex Tucker, Somerville, MA (US); Jonathan W. Kotula, Somerville, MA (US); Vincent M. Isabella, Cambridge, MA (US); Yves Millet, Newton, MA (US)

(21) Appl. No.: 15/402,147

(22) Filed: Jan. 9, 2017

Related U.S. Application Data
(63) Continuation-in-part of application No. 15/379,445, filed on Dec. 14, 2016.
Application No. 15/379,445 is a continuation-in-part of application No. PCT/US16/44922, filed on Jul. 29, 2016.
Application No. PCT/US16/44922 is a continuation-in-part of application No. PCT/US16/32565, filed on May 13, 2016.
Application No. PCT/US16/32565 is a continuation-in-part of application No. PCT/US16/37098, filed on Jun. 10, 2016.
(60) Provisional application No. 62/199,445, filed on Jul. 31, 2015.
(60) Provisional application No. 62/341,320, filed on May 25, 2016.
(60) Provisional application No. 62/336,338, filed on May 13, 2016.

(51) Int. Cl. A61K 35/74; C12N 15/70

(52) U.S. Cl. CPC A61K 35/74; C12N 15/70; A61K 2035/11

(57) ABSTRACT

The present disclosure provides engineered bacterial cells comprising a heterologous gene encoding a propionate catabolism enzyme. In another aspect, the engineered bacterial cells further comprise at least one heterologous gene encoding a transporter of propionate or a kill switch. The disclosure further provides pharmaceutical compositions comprising the engineered bacteria, and methods for treating disorders involving the catabolism of propionate, such as Propionic Acidemia and Methylmalonic Acidemia, using the pharmaceutical compositions.

### RELATED APPLICATIONS

This application is a continuation in part of PCT/US2016/044922, filed on Jul. 29, 2016, which claims priority to U.S. Provisional Patent Application No. 62/199,445, filed on Jul. 31, 2015; U.S. Provisional Patent Application No. 62/341,320, filed May 25, 2016; U.S. Provisional No. 62/336,338, filed on May 13, 2016; and is a continuation in part of International Application No. PCT/US2016/032565, filed on May 13, 2016; and's continuation in part of International Application No. PCT/US2016/037098, filed on Jun. 10, 2016; and is a continuation in part of U.S. patent application Ser. No. 15/379,445, filed on Dec. 14, 2016, the entire contents of each of which are expressly incorporated herein by reference in their entireties, including the drawings.

### SEQUENCE LISTING

The instant application contains a Sequence Listing which has been submitted electronically in ASCII format and is hereby incorporated by reference in its entirety. Said ASCII copy, created on Mar. 23, 2017, is named 126046-00603_SL.txt and is 546,069 bytes in size.

### BACKGROUND

In healthy subjects, the human body converts certain amino acids, such as isoleucine, valine, threonine, and methionine, as well as odd chain fatty acids, into propionyl CoA to create energy (FIG. 4). The enzyme propionyl CoA carboxylase (PCC) then converts propionyl CoA to methylmalonyl CoA, and the methylmalonyl CoA mutase (MUT) enzyme then converts methylmalonyl CoA into succinyl CoA, which enters the citric acid cycle and glucogenesis.

Enzyme deficiencies or mutations which lead to the toxic accumulation of propionyl CoA or methylmalonyl CoA result in the development of disorders associated with propionate catabolism, such as Propionic Acidemia (PA) and Methylmalonyl Acidemia (MMA). Severe nutritional deficiencies of Vitamin B12 can also result in MMA (Higginbottom et al., M. Engl. J. Med., 299(7):317-323, 1978). In these diseases, propionic acid or methylmalonic acid can build up in the blood stream, leading to damage of the brain, heart, and liver (FIG. 3 and FIG. 4). Clinical manifestations of the disease vary depending on the degree of enzyme deficiency and include seizures, vomiting, lethargy, hypotonia, encephalopathy, developmental delay, failure to thrive, and secondary hyperammonemia (Deodato et al., Methylmalonic and propionic aciduria, Am. J. Med. Genet. C. Semin. Med. Genet, 142(2):104-112, 2006).

Currently available treatments for disorders involving propionate catabolism are inadequate for the long term management of the disorders and have severe limitations. A low protein diet, with micronutrient and vitamin supplementation, as necessary, is the widely accepted long-term disease management strategy for many such disorders (Saudubray et al., Inborn Metabolic Diseases, Diagnosis, and Treatment, 2012). Supplementation with L-carnitine, as well as antibiotic therapy to remove intestinal propiogenic flora is also often utilized. However, dietary-intake restrictions can be particularly problematic since protein is required for metabolic activities (Baumgartner et al., Orphanet. J. Rare Dis., 9(130):1-36, 2014). Thus, even with proper monitoring and patient compliance, dietary restrictions result in a high incidence of mental retardation (Baumgartner et al., 2014). Liver transplantation has recently been considered for PA and MMA subjects (Li et al., Liver Transpl., 2015). However, the limited availability of donor organs, the costs associated with the transplantation itself, and the undesirable effects associated with continued immunosuppressant therapy limit the practicality of liver transplantation for treatment of disorders involving the catabolism of propionate. Therefore, there is significant unmet need for effective, reliable, and/or long-term treatment for disorders involving the catabolism of propionate.

### SUMMARY

The present disclosure provides engineered bacterial cells, pharmaceutical compositions thereof, nucleic acids, and methods of modulating and treating disorders involving the catabolism of propionate. Specifically, the engineered bacteria disclosed herein have been constructed to comprise genetic circuits composed of, for example, one or more propionate catabolism genes to treat the disease, as well as other optional circuitry designed to ensure the safety and non-colonization of a subject that is administered the engineered bacteria, such as, for example, auxotrophies, kill switches, and combinations thereof. These engineered bacteria are safe and well tolerated and augment the innate activities of the subject's microbiome to achieve a therapeutic effect.

In some embodiments, the disclosure provides a bacterial cell that has been genetically engineered to comprise one or more genes, gene cassettes, and/or synthetic circuits encoding a propionate catabolism enzyme or propionate catabolism pathway, and is capable of metabolizing propionate and/or other metabolites, such as propionyl CoA, methylmalonate, and/or methylmalonyl CoA. Thus, the genetically engineered bacterial cells and pharmaceutical compositions comprising the bacterial cells may be used to treat and/or prevent diseases associated with propionate catabolism, such as propionic acidemia (PA) and methylmalonic acidemia (MMA).

In some embodiments, the disclosure provides a bacterial cell that has been engineered to comprise gene sequence(s) encoding one or more propionate catabolism enzyme(s). In some embodiments, the disclosure provides a bacterial cell has been engineered to comprise gene sequence(s) encoding one or more propionate catabolism enzyme(s) and is capable of reducing the level of propionate and/or other metabolites, for example, methylmalonate, propionyl CoA and/or methylmalonyl CoA. In some embodiments, the disclosure provides a bacterial cell has been engineered to comprise gene sequence(s) encoding one or more propionate catabolism enzyme(s) that is operably linked to an inducible promoter. In some embodiments, the disclosure provides a bacterial cell has been engineered to comprise gene sequence(s) encoding one or more propionate catabolism enzyme(s) that is operably linked to a constitutive promoter. In some embodiments, the disclosure provides a bacterial cell has been engineered to comprise gene sequence(s) encoding one or more propionate catabolism enzyme(s) that is operably linked to an inducible promoter that is induced under low oxygen and/or anaerobic conditions, e.g., such as those conditions found in the mammalian gut. In some embodiments, the disclosure provides a bacterial cell has been engineered to comprise gene sequence(s) encoding one or more propionate catabolism enzyme(s) that is operably linked to an inducible promoter that is induced by environmental signals and/or conditions found in the mammalian gut (e.g., induced by metabolites or biomolecules found in the mammalian gut). In some embodiments, the disclosure provides a bacterial cell has been engineered to comprise gene sequence(s) encoding one or more propionate catabolism enzyme(s) and is capable of reducing the level of propionate and/or other metabolites, for example, methylmalonate, propionyl CoA and/or methylmalonyl CoA in low-oxygen environments, e.g., the gut. In some embodiments, the bacterial cell has been genetically engineered to comprise one or more circuits encoding one or more propionate catabolism enzyme(s) and is capable of processing and reducing levels of propionate, methylmalonate, propionyl CoA and/or methylmalonyl CoA, e.g., in low-oxygen environments, e.g., the gut. In some embodiments, the bacterial cell of the disclosure has also been genetically engineered to comprise gene sequence(s) encoding one or more transporter(s) of propionate. Thus, the genetically engineered bacterial cells and pharmaceutical compositions comprising the bacterial cells of the disclosure may be used to convert excess propionic acid, propionyl CoA, and/or methylmalonyl CoA into non-toxic molecules in order to treat and/or prevent conditions associated with disorders involving the catabolism of propionate, such as Propionic Acidemia or Methylmalonic Acidemia.

### BRIEF DESCRIPTION OF THE DRAWINGS

FIG. 1 depicts schematics of the gene organization of exemplary synthetic biotics of the disclosure for the treatment of propionic acidemia and/or methylmalonic acidemia and/or disorders characterized by propionic acidemia and/or methylmalonic acidemia. FIG. 1A depicts a schematic of the gene organization of an exemplary synthetic biotic of the disclosure comprising a gene cassette expressing the prpE, phaB, phaC, and phaA genes under the control of an inducible promoter. PrpE, PhaB, PhaC, and PhaA are capable of catabolizing propionate or propionyl CoA and/or methylmalonic acid or methylmalonyl CoA into P(HV-co-HB). Protein lysine acyltransferase is deleted to prevent inactivation of PrpE. FIG. 1B depicts a schematic of the gene organization of an exemplary synthetic biotic of the disclosure comprising a gene cassette expressing prpE, accA, pccB, mmcE, mutA and mutB as two polycistronic messages from two inducible promoters. PrpE, accA, pccB, mmcE, mutA and mutB are capable of catabolizing propionate or propionyl CoA and/or methylmalonic acid or methylmalonyl CoA into succinate, which can be utilized through the TCA cycle or exported from the cell. Protein lysine acyltransferase (pka) is deleted to prevent inactivation of PrpE. The gene sequence(s) encoded by the genetically engineered bacteria may be under the control of a constitutive and/or an inducible promoter, which may be the same or different between the circuits. Non-limiting examples of such promoters described herein.

FIG. 2 depicts various branched chain amino acid (BCAA) degradative pathways and the metabolites and associated diseases relating to BCAA metabolism.

FIG. 3 depicts the cause and symptoms of a disease associated with propionate catabolism, such as Propionic Acidemia (PA) and Methylmalonic Acidemia (MMA), which result from genetic defects in propionyl-CoA carboxylase or methylmalonyl-CoA mutase.

FIG. 4 depicts the differences between healthy (normal) human subjects, and subjects having a disease associated with propionate catabolism, such as propionic acidemia (PA).

FIGS. 5A and 5B depict schematics of the major pathway (FIG. 5A) and minor pathways (FIG. 5B) of propionate catabolism in healthy human subjects. Briefly, propionyl CoA is carboxylated to D-methylmalonyl CoA by the enzyme Propionyl CoA Carboxylase (PCC), which is isomerized to L-methylmalonyl CoA. A vitamin B₁₂-dependent enzyme, Methylmalonyl CoA Mutase (MUT) then catalyzes the rearrangement of L-methylmalonyl CoA to succinyl CoA, which is then incorporated into the citric acid cycle. Minor propionate catabolism pathways also exist and are present in subjects having diseases associated with propionate catabolism, such as PA, but these pathways are insufficient to counterbalance the lack of the major pathway. FIG. 5C depicts a schematic showing the metabolic relationship between PA and MMA. FIG. 5D depicts enzyme and other deficiencies in PA. FIG. 5E depicts enzyme and other deficiencies in MMA.

FIGS. 6A-C depicts a graph showing propionic acidemia biomarkers in PCCAA138T hypomorph mouse model as compared to a WT FVB mouse. FIG. 6A, FIG. 6B, and FIG. 6C depict graphs showing detection of blood biomarkers; propionylcarnitine/acetylcarnitine ratio (FIG. 6A), propionate concentration (FIG. 6B), and 2-methylcitrate (FIG. 6C). FIG. 6D, FIG. 6E, and FIG. 6F depict graphs showing the detection of urine biomarkers; propionyl-glycine (FIG. 6D), Tigylglycine (FIG. 6E), and 2-methylcitrate (FIG. 6F).

FIG. 7A, FIG. 7B, FIG. 7C and FIG. 7D depict bar graphs showing the levels of endogenous (FIG. 7A and FIG. 7B) and radiolabeled propionic acid (FIG. 7C and FIG. 7D) in blood, small intestine and large intestine at various time points post subcutaneous administration of isotopic propionic acid in C57BL/6J (FIG. 7A and FIG. 7C) and PCCAA138T mice (FIG. 7B and FIG. 7D). Isotopic propionic acid is seen at very low levels in the blood, small intestine, and cecum within 30 min, indicating that enterorecirculation of propionic acid is occurring.

FIGS. 8 A-D depict potential pathways that may be engineered into the bacteria in order to consume propionic acid and/or methylmalonic acid into inert end products. FIG. 8A depicts a schematic of propionate catabolism, resulting in an inert product. FIG. 8B, FIG. 8C and FIG. 8D depict schematics of three exemplary pathways, which can be utilized for propionate or methylmalonic acid catalysis. The methylmalonyl-CoA (human) pathway and the 2-methylcitrate pathway produce succinate. In some embodiments, a succinate exporter can also be expressed in the engineered bacteria. In another embodiment, the polyhydroxyalkanoate pathway can be designed and utilized, resulting in the production of polyhydroxyalkanoates in the engineered bacteria. These pathways serve as a framework for the designed propionate catabolism pathway circuits disclosed herein. FIG. 8D depicts a schematic showing a rearranged version of FIG. 8C, showing predictions for the fate of the carbon from propionic acid. For the PHA pathway, the carbon is stored as PHA polymers in the cell. In the MMCA pathway, propionate is consumed via the TCA cycle (releasing the carbon as CO2) or succinate is exported.

FIGS. 9A-B depict schematics showing the activation of propionate to propionyl CoA. FIG. 9A shows a schematic of propionate activation through PrpE. PrpE converts propionate and free CoA to propionyl-CoA in an irreversible, ATP-dependent manner, releasing AMP and PPi (pyrophosphate). PrpE can be inactivated by postranslational modification of the active site lysine. Protein lysine acetyltraferase (Pka) in E. coli carries out the propionylation of PrpE. The enzyme CobB depropionylates PrpE-Pr, making the inactivation reversible. By simply deleting the pka gene, the PrpE inactivation is eliminated altogether. In some embodiments of the disclosure, the genetically engineered bacteria comprise Δpka to prevent inactivation of PrpE and to increase activity through the downstream catabolic pathways. FIG. 9B shows a schematic of propionate activation through pct. Pct converts propionate and acetyl-CoA to propionyl-CoA and acetate in a reversible reaction.

FIGS. 10A-C depict a schematic of the polyhydroxyalkanoate pathway (FIG. 10A) and chemical structures of the polymers produced from propionate through the PHA pathway (FIG. 10B) and the gene organization of an exemplary engineered bacterium of the disclosure (FIG. 10C). The PHA pathway is a heterologous bacterial pathway used for carbon storage as polymers. In the gene circuit, the prpE, phaB, phaC, and phaA genes are expressed under the control of an inducible promoter. PrpE, PhaB, PhaC, and PhaA are capable of catabolizing propionate or propionyl CoA into polyhydroxybutyrate, polyhydroxyvalerate, or P(HV-co-HB). Specifically, PrpE, a propionate-CoA ligase, converts propionate to propionyl CoA. PhaA, a beta-ketothiolase, then converts propionyl CoA to 3-keto-valeryl-CoA or converts acetyl-CoA to acetoacetyl-CoA. PhaB, an acetoacetyl-CoA reductase, then converts acetoacetyl-CoA into 3-hydroxy-butyryl-CoA or 3-keto-valeryl-CoA to 3-hydroxy-valeryl-CoA. PhaC, a polyhydroxyalkanoate synthase converts 3-hydroxy-butyryl-CoA into polyhydroxybutyrate or 3-hydroxy-valeryl-CoA to polyhydroxyvalerate or converts polyhydroxybutyrate and polyhydroxyvalerate to P(HV-co-HB). In some embodiments, the phaBCA genes are from Acinetobacter sp RA3849 and are codon-optimized for E. coli. In some embodiments, the E. coli Nissle prpE gene and the codon-optimized phaBCA genes are under the control of an aTc-inducible promoter in a single operon. In some embodiments, the gene sequence(s) encoded by the genetically engineered bacteria may be under the control of a different inducible promoter, which may be the same or different between two operons. In some embodiments, the gene sequence(s) encoded by the genetically engineered bacteria may be under the control of a constitutive and/or an inducible promoter, which may be the same or different between two operons. Non-limiting examples of such promoters described herein.

FIG. 11 depicts a schematic of the gene organization of an exemplary construct, comprising a prpE-phaBCA gene cassette under the control of a tetracycline inducible promoter sequence, on a ˜10-copy, kanamycin-resistant plasmid. In some embodiments, the gene sequence(s) shown may be under the control of a constitutive and/or an inducible promoter. Non-limiting examples of such promoters described herein.

FIG. 12 depicts a graph showing propionate concentrations over time in samples comprising genetically engineered bacteria expressing the polyhydroxyalkanoate (PHA) pathway on a ˜10-copy plasmid, as compared to wild type Nissle controls, in the presence and absence of the inducer molecule. Bacteria were induced with ATC (or left uninduced), and then grown in culture medium supplemented to an OD600 of 2.0. Samples were harvested by centrifugation and resuspended in M9 minimal media. The activity of resuspended samples was measured by inoculating samples into M9 minimal media supplemented with glucose and sodium propionate (3 mM) to an OD600 of 1.0. Samples were removed at 0 hrs, 1.5, 3, and 4.5 hrs post-inoculation, and propionate concentrations were determined by mass spectrometry. The graph depicts propionate consumption by the polyhydroxyalkanoate circuit design for the engineered bacteria (SYN-PHA) in the induced as compared to wild type Nissle. Propionate assay was initiated with ˜10⁹ cfu/ml pre-induced bacteria and the propionate consumption rate was ˜1.4 umol hr-1 per 10⁹ cells.

FIGS. 13A-C depict graphs showing propionate (FIG. 13A), acetate (FIG. 13B) and butyrate (FIG. 13C) concentrations over time in samples comprising genetically engineered bacteria expressing the polyhydroxyalkanoate (PHA) pathway on a ˜10 copy plasmid (SYN-PHA), as compared to wild type Nissle controls, both in the presence of the inducer molecule. The PHA assay was performed in a mixture of short chain fatty acids to mimic the colon ratios (propionate:acetate:butyrate, approximately 6:10:4). Bacteria were induced with ATC (or left uninduced), and then grown in culture medium supplemented to an OD600 of 2.0. Samples were harvested by centrifugation and resuspended in M9 minimal media. The activity of resuspended samples was measured by inoculating samples into M9 minimal media supplemented with glucose and sodium propionate (6 mM), acetate (10 mM), and butyrate (4 mM) to an OD600 of 1.0. Samples were removed at 0 hrs, 1.5, 3, and 4.5 hrs post-inoculation, and propionate concentrations were determined by mass spectrometry. The data show that propionate consumption rate is consistent in the presence or absence of acetate and butyrate, and that the PHA pathway does not significantly affect acetate and butyrate concentrations.

FIG. 14A-FIG. 14H depict schematic representations of propionate catabolism constructs (FIG. 14A, FIG. 14C, FIG. 14E, and FIG. 14G) and graphs showing propionate concentrations over time (FIG. 14B, FIG. 14D, FIG. 14F, and FIG. 14H). The samples analyzed comprise genetically engineered bacteria expressing an inducible polyhydroxyalkanoate (PHA) cassette (ptet-prpE-phaBCA) on a ˜10 copy plasmid (SYN-PHA), in the presence of the inducer molecule. These strains were further supplemented with an second plasmid (˜15-copies) expressing one of the genes, i.e., prpE, phaB, phaC, and phaB, under the control of an inducible promoter, i.e., an arabinose inducible promoter. In this assay, either the prpE-phaBCA operon alone, or both the prpE-phaBCA plasmid and the arabinose inducible plasmid carrying the second copy of one of the operon genes were induced. Wild type Nissle was included for reference. Bacteria were induced with ATC or ATC and arabinose (or left uninduced), and then grown in culture medium supplemented to an OD600 of 2.0. Samples were harvested by centrifugation and resuspended in M9 minimal media. The activity of resuspended samples was measured by inoculating samples into M9 minimal media supplemented with glucose and sodium propionate (3 mM) to an OD600 of 1.0. Samples were removed at 0 hrs, 1.5, 3, and 4.5 hrs post-inoculation, and propionate concentrations were determined by mass spectrometry. The graph shows that the rate of propionate consumption is increased most significantly when more phaC is expressed, suggesting that the pathway is improved by increasing the PhaC levels from the original prpE-phaBCA plasmid. This can for example be accomplished by increasing the translation rate by employing a stronger ribosome binding site in front of the phaC gene. Alternatively, an additional copy of the gene may be added to the same or an additional circuit. In some embodiments, the genetically engineered bacteria comprise a prpE-phaBCA operon, in which PhaC levels are increased through the utilization of a strong ribosome binding site (RBS). In some embodiments, the genetically engineered bacteria comprising a prpE-phaBCA operon further comprise an additional copy of phaC. In other embodiments, the tetracycline promoter shown in the genetic circuits is replaced with a different inducible or constitutive promoter. Non-limiting examples of such promoters described herein.

FIGS. 15A-15C depict schematics of the methylmalonyl-CoA pathway and exemplary methylmalonyl CoA circuit designs. FIG. 15A depicts a schematic showing PrpE reaction and by the methylmalonyl CoA pathway, in which the products of the prpE, pccB, accA1, mmcE, mutA, and mutB genes convert propionate into succinate, and which can be used for circuit design. The methylmalonyl-CoA pathway carries out reactions homologous to those in the mammalian pathway and the pathway is assembled from heterologous bacterial enzymes. In one embodiment, genes accA (from Streptomyces coelicolor), pccB (from Streptomyces coelicolor), mmcE (from Propionibacterium freudenreichii), and mutAB (from Propionibacterium freudenreichii) were used and codon-optimized for expression in E. coli Nissle. FIG. 15B depicts a schematic showing an exemplary circuit design of the disclosure, in which the genetically engineered bacteria comprise a gene cassette comprising the prpE, pccB, accA1, mmcE, mutA, and mutB genes under the control of an inducible promoter, e.g., a aTc-inducible promoter. FIG. 15C depicts a schematic showing an exemplary circuit design of the disclosure, in which the genetically engineered bacteria comprise a cassette comprising prpE, pccB, accA1, under the control of a first inducible promoter, e.g., Ptet (aTc inducible) and a second cassette comprising mmcE and mutAB under the control of a second inducible promoter, e.g., Para (arabinose inducible). Induction of the pathway requires the addition of aTc and arabinose. In either circuit (FIG. 15B or FIG. 15C), a succinate exporter may also be expressed in the engineered bacteria, the tetracycline promoter shown in the genetic circuits is replaced with a different inducible or constitutive promoter. Non-limiting examples of such promoters described herein.

FIGS. 16A-B depict schematics of the gene organization of exemplary constructs. FIG. 16A depicts a schematic of the gene organization of an exemplary construct, comprising a mmcE-mutA-mutB gene cassette under the control of an arabinose inducible promoter sequence, on a ˜15-copy, ampicillin-resistant plasmid. FIG. 16B depicts a schematic of the gene organization of an exemplary construct, comprising a prpE-accA-pccB gene cassette under the control of a tetracycline inducible promoter sequence, on a ˜10-copy, kanamycin-resistant plasmid.

FIGS. 17A-F depict schematics of the MMCA pathway combined with a succinate exporter and related exemplary genetic circuits and synthetic biotics. FIG. 17A depicts a schematic of propionate and/or methylmalonic acid catabolism through the MMCA pathway. The resulting succinate can be metabolized through the TCA cycle or removed from the bacterial cell through an exporter. Exemplary exporters include sucE1 succinate exporter (e.g., from Corynebacterium glutamicum) and/or the native Nissle succinate exporter dcuC. FIG. 17B depicts an exemplary circuit or gene cassette for the expression of the sucE1 succinate exporter (e.g., from Corynebacterium glutamicum) under the control of an inducible promoter, e.g., an arabinose-inducible promoter. This construct can either be expressed in the synthetic biotic on a plasmid, or it can be integrated into the genome. For example, a knock-in of the construct, which deletes the araBA genes and part of the araD gene, can be performed, which eliminates metabolism of arabinose by E. coli. FIG. 17C depicts a schematic of the gene organization of an exemplary synthetic biotic of the disclosure comprising a gene cassette expressing the prpE, phaB, phaC, and phaA genes under the control of an inducible promoter. The synthetic biotic further comprises a gene cassette expressing the sucE1 gene under the control of an inducible promoter. In other embodiments, the promoters are constitutive promoters. Non-limiting examples of such promoters described herein. FIG. 17D depicts a schematic of a construct comprising the sucE1 succinate exporter (from Corynebacterium glutamicum). FIG. 17E depicts a schematic of a construct comprising the E. coli dcuC succinate transporter. FIG. 17F depicts a schematic of a construct comprising or comprising both sucE1 and dcuC transporters.

FIG. 18 depicts a graph showing propionate concentrations over time in samples comprising genetically engineered bacteria expressing the methylmalonyl-CoA pathway circuit (SYN-MMCA) or a polyhydroxyalkanoate pathway circuit (SYN-PHA) on a ˜10- and ˜15-copy plasmids as compared to wild type Nissle controls, in the presence of the inducer molecule. Bacteria were induced ATC or ATC and arabinose (or left uninduced), and then grown in culture medium supplemented to an OD600 of 2.0. Samples were harvested by centrifugation and resuspended in M9 minimal media. The activity of resuspended samples was measured by inoculating samples into M9 minimal media supplemented with glucose and sodium propionate (3 mM) to an OD600 of 1.0. Samples were removed at were removed at 0 hrs, 1.5, 3, 4.5, and 18 hrs post-inoculation, cells were removed, and propionate concentrations were determined by mass spectrometry. The graph depicts propionate consumption by the methylmalonyl-CoA pathway or a polyhydroxyalkanoate circuit design for the engineered bacteria in the induced as compared to wild type Nissle. Propionate assay was initiated with ˜109 cfu/ml pre-induced bacteria and the propionate consumption rate was ˜3.8 μmol/hr/10⁹ bacteria in the strain expressing the methylmalonyl-CoA pathway circuit.

FIG. 19 depicts one example of a normal pathway for the catabolism of propionate via the methylcitrate cycle in bacteria, for example, E. coli. Briefly, PrpE, a Propionate-CoA ligase, converts propionate to propionyl CoA. PrpC, a 2-methylcitrate synthetase, then converts propionyl CoA to 2-methylcitrate. PrpD, a 2-methylcitrate dehydrogenase, then converts 2-methylcitrate into 2-methyisocitrate, and PrpB, a 2-methylisocitrate lyase, converts 2-methyisocitrate into succinate and pyruvate.

FIGS. 20A-20C depict schematics of the 2-methylcitrate cycle in bacteria, e.g., E. coli, (FIG. 20A) and a schematic of the gene organization of an exemplary engineered bacterium (FIG. 20B). In the circuit, the prpB, prpC, prpD, and prpE genes are expressed under the control of an inducible promoter in order to produce succinate and pyruvate. In some embodiments, a succinate exporter may also be expressed in the engineered bacteria. In some embodiments, a constitutive promoter may drive the expression of the circuit shown and/or the succinate exporter. In some embodiments, an inducible promoter may drive the expression of the circuit shown and/or the succinate exporter. Non-limiting examples of such promoters described herein. FIG. 20C depicts a schematic of the gene organization of an exemplary construct, comprising a prpBCDE gene cassette under the control of a tetracycline inducible promoter sequence, on a ˜10-copy, kanamycin-resistant plasmid.

FIGS. 21A-21G depict schematics of the exemplary gene organization synthetic biotics of the disclosure for the treatment of propionic acidemia and/or methylmalonic acidemia and/or disorders characterized by propionic acidemia and/or methylmalonic acidemia. The gene sequence(s) encoded by the genetically engineered bacteria may be under the control of a constitutive and/or an inducible promoter, which may be the same or different between the circuits. Non-limiting examples of such promoters described herein. FIG. 21A depicts a schematic of the gene organization of an exemplary synthetic biotic of the disclosure comprising a gene cassette expressing the prpE, phaB, phaC, and phaA genes under the control of an inducible promoter. PrpE, PhaB, PhaC, and PhaA are capable of catabolizing propionate or propionyl CoA and/or methylmalonic acid or methylmalonyl CoA into P(HV-co-HB). Protein lysine acyltransferase (pka) is deleted to prevent inactivation of PrpE. In certain embodiments, the prpE-phaBCA circuit is further modified by adding a strong RBS upstream of the phaC translation start site. In other embodiments, synthetic biotic comprised multiple copies of the PhaC gene. In some embodiments, the PhaC gene is located immediately distal to the promoter, as the rest of genes in the cassette, to ensure the greatest number of transcripts. T7 polymerase may produce incomplete polycistronic transcripts (prematurely terminated). FIG. 21B depicts a schematic of the gene organization of an synthetic biotic of FIG. 1A or FIG. 21A, with the addition of a ThyA auxotrophy. FIG. 21C depicts the gene organization of the synthetic biotic of FIG. 1B, with the addition of a ThyA auxotrophy. FIG. 21D depicts a schematic of the gene organization of an exemplary synthetic biotic of the disclosure comprising a gene cassette expressing prpE, accA, pccB, mmcE, mutA and mutB as two polycistronic messages from two inducible promoters. PrpE, accA, pccB, mmcE, mutA and mutB are capable of catabolizing propionate or propionyl CoA and/or methylmalonic acid or methylmalonyl CoA into succinate, which can be utilized through the TCA cycle or exported from the cell. Protein lysine acyltransferase (pka) is deleted to prevent inactivation of PrpE. In some embodiments, the synthetic biotic comprises a SucE1 and/or dcuC exporter cassette, as described herein. FIG. 21E depicts a schematic of a synthetic biotic comprising one or more of two different gene cassettes for propionate catabolism (PHA and MMCA pathway cassettes). FIG. 21F depicts a schematic of the gene organization of an exemplary synthetic biotic of the disclosure comprising a gene cassette expressing prpE, accA, pccB, mmcE, mutA and mutB as two polycistronic messages from two inducible promoters in combination with MatB. Protein lysine acyltransferase (pka) is deleted to prevent inactivation of PrpE. In some embodiments, the synthetic biotic comprises a SucE1 and/or dcuC exporter cassette, as described herein. FIG. 21G depicts a schematic of the gene organization of a synthetic biotic comprising one or more of two different gene cassettes for propionate catabolism (PHA and MMCA pathway cassettes) in combination with MatB.

FIG. 22 depict graphs showing PC/AC ratio in plasma of PCCAA138T hypomorph mice gavaged with a strain expressing PHA pathway genes (PHA), a strain expressing MMCA (MMCA) pathway genes or streptomycin resistant Nissle (wild type) as compared to H₂O only controls. Both strains reduce C3/C2 ratios >50%. The PHA pathway strain comprises a plasmid with pTet-prpE-phaB-phaC-phaA, The MMCA strain comprises a low copy plasmid comprising ptet-prpE-pccB-accA1 and a second low copy plasmid comprising pAra-mmcE-mutA-mutB as described in Example 12 and shown in FIG. 15C, and FIG. 16A and FIG. 16B.

FIG. 23A-FIG. 23D depict graphs showing propionic acidemia biomarkers in blood (FIG. 23A) and urine (FIGS. 23B, 23C, and 23D) in PCCAA138T hypomorph mice fed with high protein chow gavaged with a strain expressing PHA pathway genes (PHA), a strain expressing MMCA (MMCA) pathway genes or streptomycin resistant Nissle (wild type) as compared to H2O only controls. The PHA pathway strain comprises a low copy plasmid with pTet-prpE-phaB-phaC-phaA. The MMCA strain comprises a low copy plasmid comprising ptet-prpE-pccB-accA1 and a second low copy plasmid comprising pAra-mmcE-mutA-mutB.

FIG. 24A depicts another non-limiting embodiment of the disclosure, wherein the expression of a heterologous gene is activated by an exogenous environmental signal. In the absence of arabinose, the AraC transcription factor adopts a conformation that represses transcription. In the presence of arabinose, the AraC transcription factor undergoes a conformational change that allows it to bind to and activate the ParaBAD promoter (ParaBAD), which induces expression of the Tet repressor (TetR) and an anti-toxin. The anti-toxin builds up in the recombinant bacterial cell, while TetR prevents expression of a toxin (which is under the control of a promoter having a TetR binding site). However, when arabinose is not present, both the anti-toxin and TetR are not expressed. Since TetR is not present to repress expression of the toxin, the toxin is expressed and kills the cell. FIG. 24B depicts graph showing toxin and anti-toxin protein levels in the presence or absence of arabinose for the embodiment described in FIG. 24A. FIG. 24C also depicts another non-limiting embodiment of the disclosure, wherein the expression of an essential gene not found in the recombinant bacteria is activated by an exogenous environmental signal. In the absence of arabinose, the AraC transcription factor adopts a conformation that represses transcription of the essential gene under the control of the araBAD promoter and the bacterial cell cannot survive. In the presence of arabinose, the AraC transcription factor undergoes a conformational change that allows it to bind to and activate the araBAD promoter, which induces expression of the essential gene and maintains viability of the bacterial cell. FIG. 24D depicts graph showing protein levels expressed from the essential gene in the presence or absence of arabinose for the embodiment described in FIG. 24C.

FIG. 24E depicts a non-limiting embodiment of the disclosure, where an anti-toxin is expressed from a constitutive promoter, and expression of a heterologous gene is activated by an exogenous environmental signal. In the absence of arabinose, the AraC transcription factor adopts a conformation that represses transcription. In the presence of arabinose, the AraC transcription factor undergoes a conformational change that allows it to bind to and activate the araBAD promoter, which induces expression of TetR, thus preventing expression of a toxin. However, when arabinose is not present, TetR is not expressed, and the toxin is expressed, eventually overcoming the anti-toxin and killing the cell. The constitutive promoter regulating expression of the anti-toxin should be a weaker promoter than the promoter driving expression of the toxin. The araC gene is under the control of a constitutive promoter in this circuit.

FIG. 24F depicts another non-limiting embodiment of the disclosure, wherein the expression of a heterologous gene is activated by an exogenous environmental signal. In the absence of arabinose, the AraC transcription factor adopts a conformation that represses transcription. In the presence of arabinose, the AraC transcription factor undergoes a conformational change that allows it to bind to and activate the araBAD promoter, which induces expression of the Tet repressor (TetR) and an anti-toxin. The anti-toxin builds up in the recombinant bacterial cell, while TetR prevents expression of a toxin (which is under the control of a promoter having a TetR binding site). However, when arabinose is not present, both the anti-toxin and TetR are not expressed. Since TetR is not present to repress expression of the toxin, the toxin is expressed and kills the cell. The araC gene is either under the control of a constitutive promoter or an inducible promoter (e.g., AraC promoter) in this circuit.

FIG. 25 depicts one non-limiting embodiment of the disclosure, where an exogenous environmental condition or one or more environmental signals activates expression of a heterologous gene and at least one recombinase from an inducible promoter or inducible promoters. The recombinase then flips a toxin gene into an activated conformation, and the natural kinetics of the recombinase create a time delay in expression of the toxin, allowing the heterologous gene to be fully expressed. Once the toxin is expressed, it kills the cell.

FIG. 26 depicts one non-limiting embodiment of the disclosure, where an exogenous environmental condition or one or more environmental signals activates expression of a heterologous gene and at least one recombinase from an inducible promoter or inducible promoters. The recombinase then flips a toxin gene into an activated conformation, and the natural kinetics of the recombinase create a time delay in expression of the toxin, allowing the heterologous gene to be fully expressed. Once the toxin is expressed, it kills the cell.

FIG. 27 depicts another non-limiting embodiment of the disclosure, where an exogenous environmental condition or one or more environmental signals activates expression of a heterologous gene and at least one recombinase from an inducible promoter or inducible promoters. The recombinase then flips at least one excision enzyme into an activated conformation. The at least one excision enzyme then excises one or more essential genes, leading to senescence, and eventual cell death. The natural kinetics of the recombinase and excision genes cause a time delay, the kinetics of which can be altered and optimized depending on the number and choice of essential genes to be excised, allowing cell death to occur within a matter of hours or days. The presence of multiple nested recombinases can be used to further control the timing of cell death.

FIG. 28 depicts a schematic of one non-limiting embodiment of the disclosure, in which the genetically engineered bacteria produces equal amount of a Hok toxin and a short-lived Sok anti-toxin. When the cell loses the plasmid, the anti-toxin decays, and the cell dies. In the upper panel, the cell produces equal amounts of toxin and anti-toxin and is stable. In the center panel, the cell loses the plasmid and anti-toxin begins to decay. In the lower panel, the anti-toxin decays completely, and the cell dies.

FIG. 29 depicts one non-limiting embodiment of the disclosure, where an exogenous environmental condition or one or more environmental signals activates expression of a heterologous gene and a first recombinase from an inducible promoter or inducible promoters. The recombinase then flips a second recombinase from an inverted orientation to an active conformation. The activated second recombinase flips the toxin gene into an activated conformation, and the natural kinetics of the recombinase create a time delay in expression of the toxin, allowing the heterologous gene to be fully expressed. Once the toxin is expressed, it kills the cell.

FIG. 30 depicts an example of a genetically engineered bacteria that comprises a plasmid that has been modified to create a host-plasmid mutual dependency, such as the GeneGuard system described in more detail herein.

FIG. 31 depicts an exemplary schematic of the E. coli 1917 Nissle chromosome comprising multiple mechanisms of action (MoAs). A single synthetic biotic may have multiple mechanisms of action (MOAs) based on the insertion of multiple copies of the same synthetic circuit or the insertion of different synthetic circuits at different sites in a bacterial chromosome.

FIG. 32 depicts a map of integration sites within the E. coli Nissle chromosome. These sites indicate regions where circuit components may be inserted into the chromosome without interfering with essential gene expression. Backslashes (/) are used to show that the insertion will occur between divergently or convergently expressed genes. Insertions within biosynthetic genes, such as thyA, can be useful for creating nutrient auxotrophies. In some embodiments, an individual circuit component is inserted into more than one of the indicated sites.

FIG. 33 depicts three bacterial strains which constitutively express red fluorescent protein (RFP). In strains 1-3, the rfp gene was inserted into different sites in the bacterial chromosome, and resulted in varying degrees of brightness under fluorescent light. Unmodified E. coli Nissle (strain 4) is non-fluorescent.

FIG. 34A and FIG. 34B depict graphs of Nissle residence in vivo. FIG. 34A depicts a graph of Nissle residence in vivo. Streptomycin-resistant Nissle was administered to mice via oral gavage without antibiotic pre-treatment. Fecal pellets from six total mice were monitored post-administration to determine the amount of administered Nissle still residing within the mouse gastrointestinal tract. The bars represent the number of bacteria administered to the mice. The line represents the number of Nissle recovered from the fecal samples each day for 10 consecutive days. FIG. 34B depicts a bar graph of residence over time for streptomycin resistant Nissle in various compartments of the intestinal tract at 1, 4, 8, 12, 24, and 30 hours post gavage. Mice were treated with approximately 109 CFU, and at each timepoint, animals (n=4) were euthanized, and intestine, cecum, and colon were removed. The small intestine was cut into three sections, and the large intestine and colon each into two sections. Intestinal effluents gathered and CFUs in each compartment were determined by serial dilution plating.

FIG. 35A, and FIG. 35B depict bar graphs if Nissle residence in vivo. FIG. 35A depicts a graph showing bacterial cell growth of a Nissle thyA auxotroph strain (thyA knock-out) in various concentrations of thymidine. A chloramphenicol-resistant Nissle thyA auxotroph strain was grown overnight in LB+10 mM thymidine at 37 C. The next day, cells were diluted 1:100 in 1 mL LB+10 mM thymidine, and incubated at 37 C for 4 hours. The cells were then diluted 1:100 in 1 mL LB+varying concentrations of thymidine in triplicate in a 96-well plate. The plate is incubated at 37 C with shaking, and the OD600 is measured every 5 minutes for 720 minutes. This data shows that Nissle thyA auxotroph does not grow in environments lacking thymidine. FIG. 35B depicts a bar graph of Nissle residence in vivo of wildtype Nissle versus Nissle thyA auxotroph (thyA knock-out). Streptomycin-resistant Nissle (wildtype or thyA auxotroph) was administered to mice via oral gavage without antibiotic pre-treatment. Fecal pellets from 6 total mice were monitored post-administration to determine the amount of administered Nissle still residing within the mouse gastrointestinal tract. Each bar represents the number of Nissle recovered from the fecal samples each day for 7 consecutive days. There were no bacteria recovered in fecal samples from mice gavaged with Nissle thyA auxotroph bacteria after day 3. This data shows that the Nissle thyA auxotroph does not persist in vivo in mice.

FIG. 36A depicts a schematic of a secretion system based on the flagellar type III secretion in which an incomplete flagellum is used to secrete a therapeutic peptide of interest (star) by recombinantly fusing the peptide to an N-terminal flagellar secretion signal of a native flagellar component so that the intracellularly expressed chimeric peptide can be mobilized across the inner and outer membranes into the surrounding host environment.

FIG. 36B depicts a schematic of a type V secretion system for the extracellular production of recombinant proteins in which a therapeutic peptide (star) can be fused to an N-terminal secretion signal, a linker and the beta-domain of an autotransporter. In this system, the N-terminal signal sequence directs the protein to the SecA-YEG machinery which moves the protein across the inner membrane into the periplasm, followed by subsequent cleavage of the signal sequence. The beta-domain is recruited to the Bam complex where the beta-domain is folded and inserted into the outer membrane as a beta-barrel structure. The therapeutic peptide is then thread through the hollow pore of the beta-barrel structure ahead of the linker sequence. The therapeutic peptide is freed from the linker system by an autocatalytic cleavage or by targeting of a membrane-associated peptidase (scissors) to a complementary protease cut site in the linker.

FIG. 36C depicts a schematic of a type I secretion system, which translocates a passenger peptide directly from the cytoplasm to the extracellular space using HlyB (an ATP-binding cassette transporter); HlyD (a membrane fusion protein); and TolC (an outer membrane protein) which form a channel through both the inner and outer membranes. The secretion signal-containing C-terminal portion of HlyA is fused to the C-terminal portion of a therapeutic peptide (star) to mediate secretion of this peptide.

FIG. 36D depicts a schematic of the outer and inner membranes of a gram-negative bacterium, and several deletion targets for generating a leaky or destabilized outer membrane, thereby facilitating the translocation of a therapeutic polypeptides to the extracellular space, e.g., therapeutic polypeptides of eukaryotic origin containing disulphide bonds. Deactivating mutations of one or more genes encoding a protein that tethers the outer membrane to the peptidoglycan skeleton, e.g., lpp, ompC, ompA, ompF, tolA, tolB, pal, and/or one or more genes encoding a periplasmic protease, e.g., degS, degP, nlpl, generates a leaky phenotype. Combinations of mutations may synergistically enhance the leaky phenotype.

FIG. 36E depicts a modified type 3 secretion system (T3SS) to allow the bacteria to inject secreted therapeutic proteins into the gut lumen. An inducible promoter (small arrow, top), e.g. a FNR-inducible promoter, drives expression of the T3 secretion system gene cassette (3 large arrows, top) that produces the apparatus that secretes tagged peptides out of the cell. An inducible promoter (small arrow, bottom), e.g. a FNR-inducible promoter, drives expression of a regulatory factor, e.g. T7 polymerase, that then activates the expression of the tagged therapeutic peptide (hexagons).

FIG. 37A, FIG. 37B, and FIG. 37C depict schematics of the gene organization of exemplary circuits of the disclosure for the expression of therapeutic polypeptides, which are secreted using components of the flagellar type III secretion system. A therapeutic polypeptide of interest, such as a propionate catabolism enzyme, is assembled behind a fliC-5′UTR, and is driven by the native fliC and/or fliD promoter (FIG. 37A and FIG. 37B) or a Tet-inducible promoter (FIG. 37C). In alternate embodiments, an inducible promoter such as oxygen level-dependent promoters (e.g., FNR-inducible promoter), and promoters induced by a metabolite that may or may not be naturally present (e.g., can be exogenously added) in the gut, e.g., arabinose can be used. The therapeutic polypeptide of interest is either expressed from a plasmid (e.g., a medium copy plasmid) or integrated into fliC loci (thereby deleting all or a portion of fliC and/or fliD). Optionally, an N terminal part of FliC is included in the construct, as shown in FIG. 37B and FIG. 37C.

FIG. 38A and FIG. 38B depict schematics of the gene organization of exemplary circuits of the disclosure for the expression of therapeutic polypeptides, which are secreted via a diffusible outer membrane (DOM) system. The therapeutic polypeptide of interest is fused to a prototypical N-terminal Sec-dependent secretion signal or Tat-dependent secretion signal, which is cleaved upon secretion into the periplasmic space. Exemplary secretion tags include sec-dependent PhoA, OmpF, OmpA, cvaC, and Tat-dependent tags (TorA, FdnG, DmsA). In certain embodiments, the genetically engineered bacteria comprise deletions in one or more of lpp, pal, tolA, and/or nlpl. Optionally, periplasmic proteases are also deleted, including, but not limited to, degP and ompT, e.g., to increase stability of the polypeptide in the periplasm. A FRT-KanR-FRT cassette is used for downstream integration. Expression is driven by a Tet promoter (FIG. 38A) or an inducible promoter, such as oxygen level-dependent promoters (e.g., FNR-inducible promoter, FIG. 38B), and promoters induced by a metabolite that may or may not be naturally present (e.g., can be exogenously added) in the gut, e.g., arabinose.

FIG. 39A depicts a “Oxygen bypass switch” useful for aerobic pre-induction of a strain comprising one or proteins of interest (POI), e.g., one or more propionate catabolism enzyme(s) (POI1) and/or one or more transporter(s)/importer(s) and/or exporter(s) (POI2) under the control of a low oxygen FNR promoter in vitro in a culture vessel (e.g., flask, fermenter or other vessel, e.g., used during with cell growth, cell expansion, fermentation, recovery, purification, formulation, and/or manufacture). In some embodiments, it is desirable to pre-load a strain with active propionate catabolism enzyme(s) prior to administration. This can be done by pre-inducing the expression of these enzymes as the strains are propagated, (e.g., in flasks, fermenters or other appropriate vesicles) and are prepared for in vivo administration. In some embodiments, strains are induced under anaerobic and/or low oxygen conditions, e.g. to induce FNR promoter activity and drive expression of one or more proteins of interest. In some embodiments, it is desirable to prepare, pre-load and pre-induce the strains under aerobic or microaerobic conditions with one or more proteins of interest. This allows more efficient growth and, in some cases, reduces the build-up of toxic metabolites.

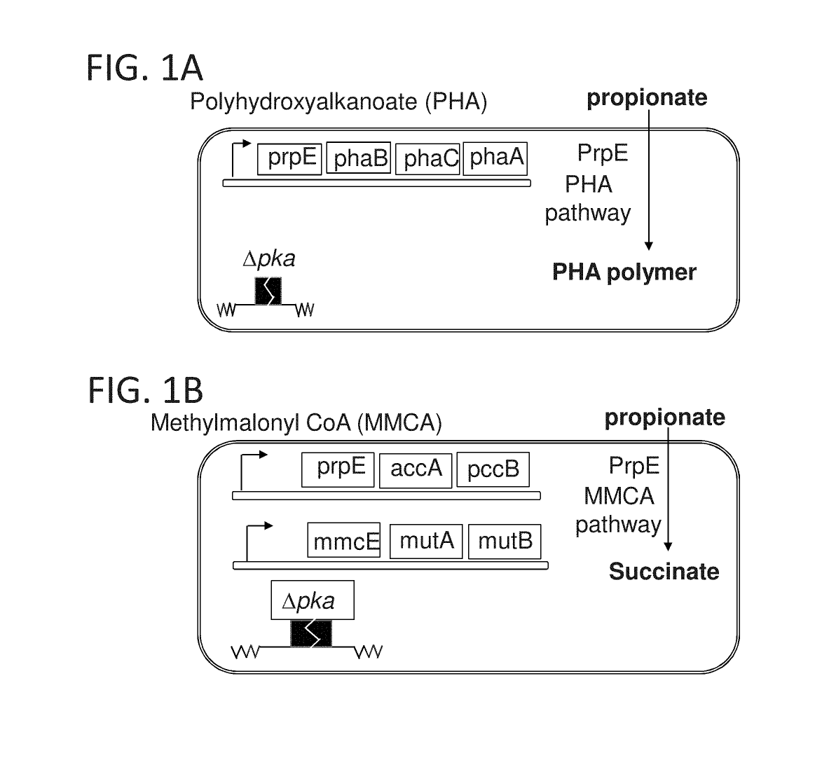

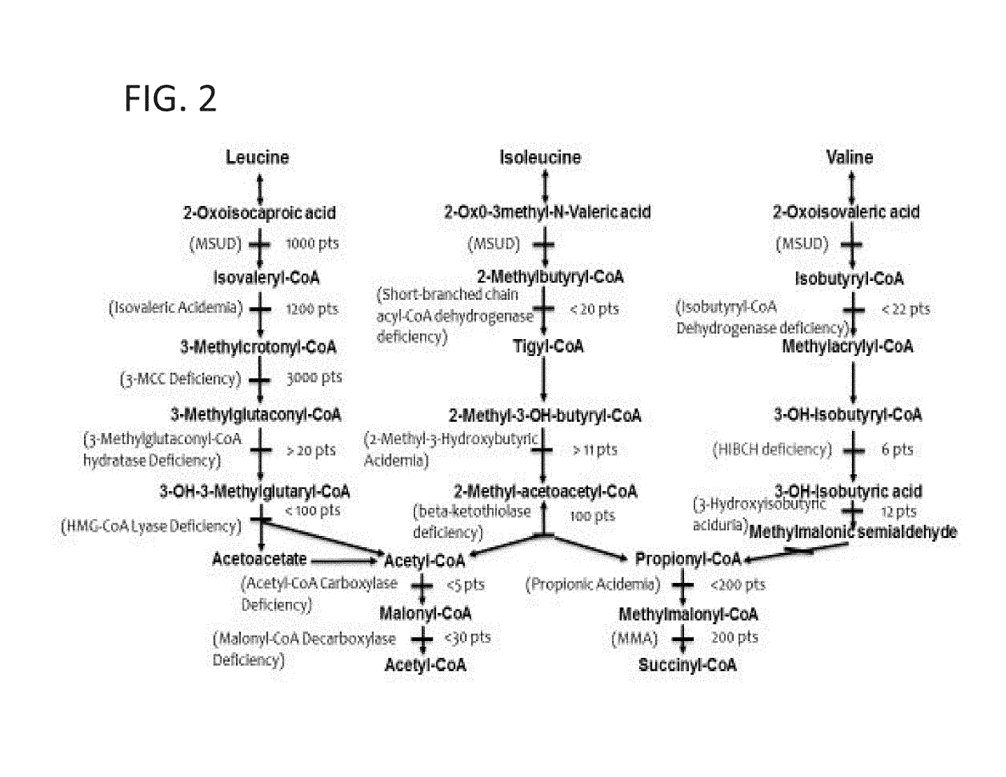

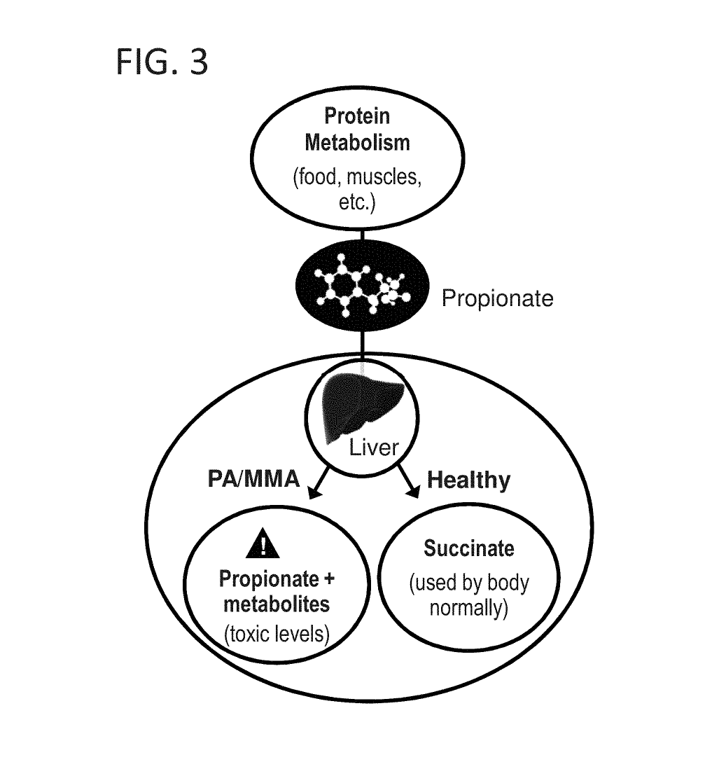

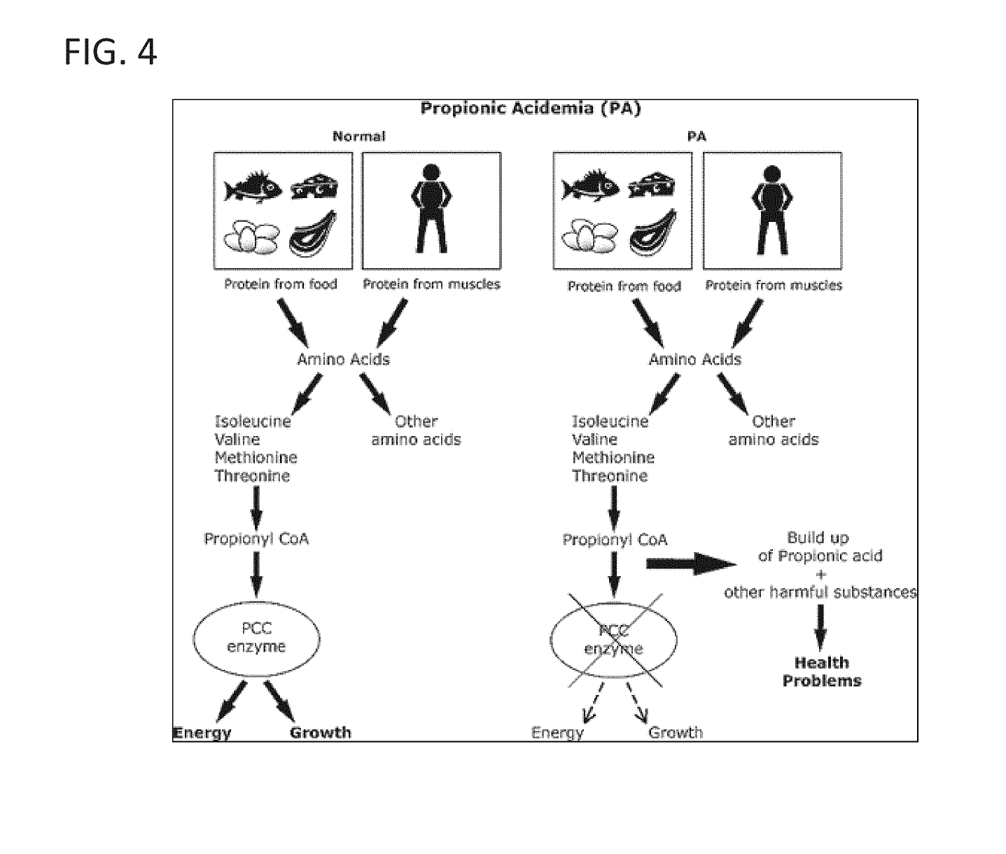

FNRS24Y is a mutated form of FNR which is more resistant to inactivation by oxygen, and therefore can activate FNR promoters under aerobic conditions (see e.g., Jervis A J, The O2 sensitivity of the transcription factor FNR is controlled by Ser24 modulating the kinetics of [4Fe-4S] to [2Fe-2S] conversion, Proc Natl Acad Sci USA. 2009 March 24; 106(12):4659-64, the contents of which is herein incorporated by reference in its entirety). The 02 sensitivity of the transcription factor FNR is controlled by Ser24 modulating the kinetics of [4Fe-4S] to [2Fe-2S] conversion, Proc Natl Acad Sci USA. 2009 Mar. 24; 106(12):4659-64, the contents of which is herein incorporated by reference in its entirety). In this oxygen bypass system, FNRS24Y is induced by addition of arabinose and then drives the expression of one or more POIs by binding and activating the FNR promoter under aerobic conditions. Thus, strains can be grown, produced or manufactured efficiently under aerobic conditions, while being effectively pre-induced and pre-loaded, as the system takes advantage of the strong FNR promoter resulting in of high levels of expression of one or more POIs. This system does not interfere with or compromise in vivo activation, since the mutated FNRS24Y is no longer expressed in the absence of arabinose, and wild type FNR then binds to the FNR promoter and drives expression of the POIs in vivo.

In some embodiments, a Lad promoter and IPTG induction are used in this system (in lieu of Para and arabinose induction). In some embodiments, a rhamnose inducible promoter is used in this system. In some embodiments, a temperature sensitive promoter is used to drive expression of FNRS24Y.

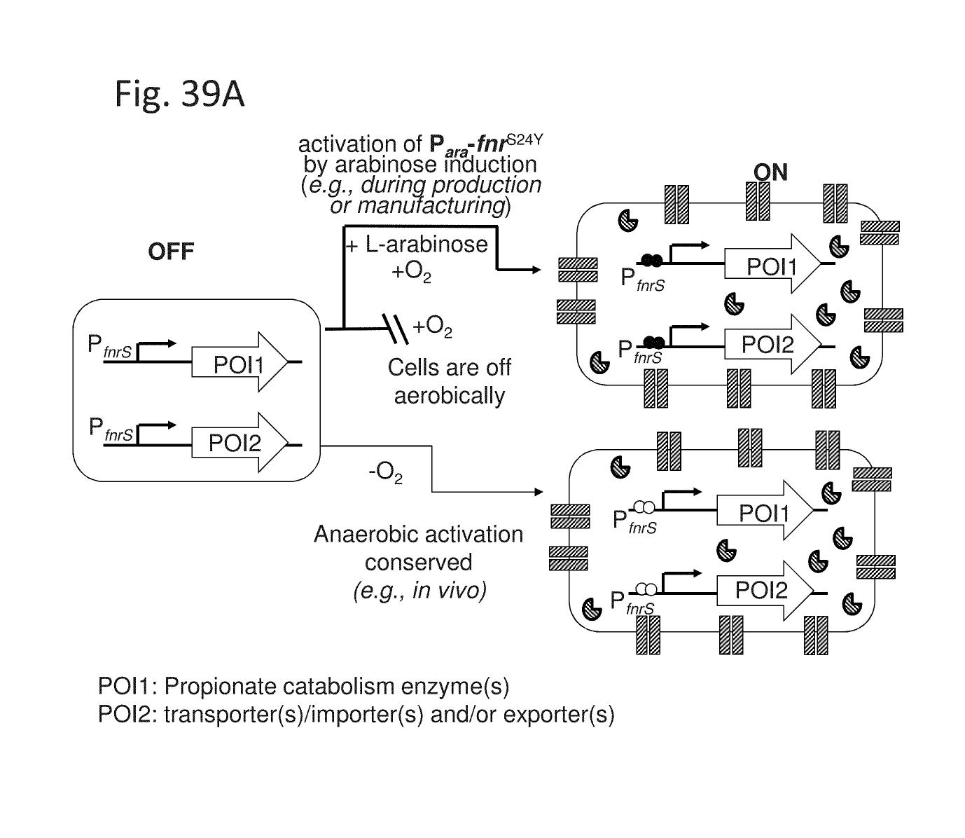

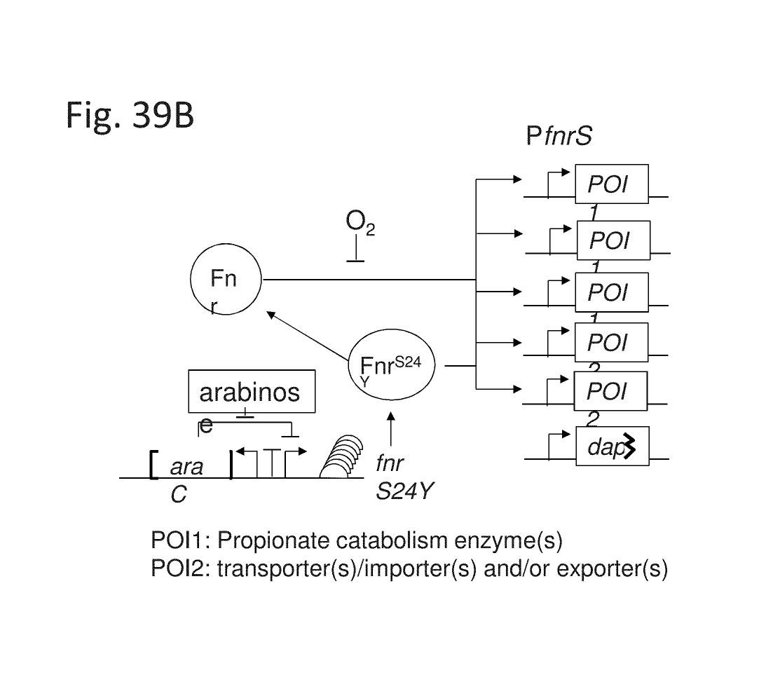

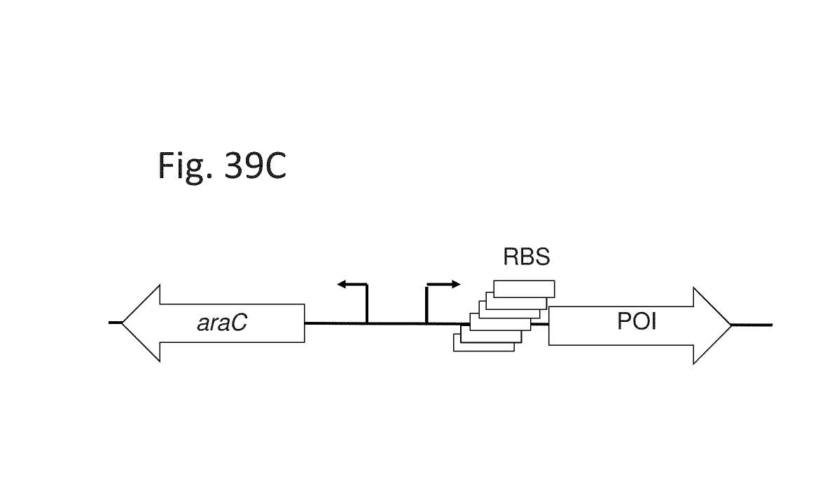

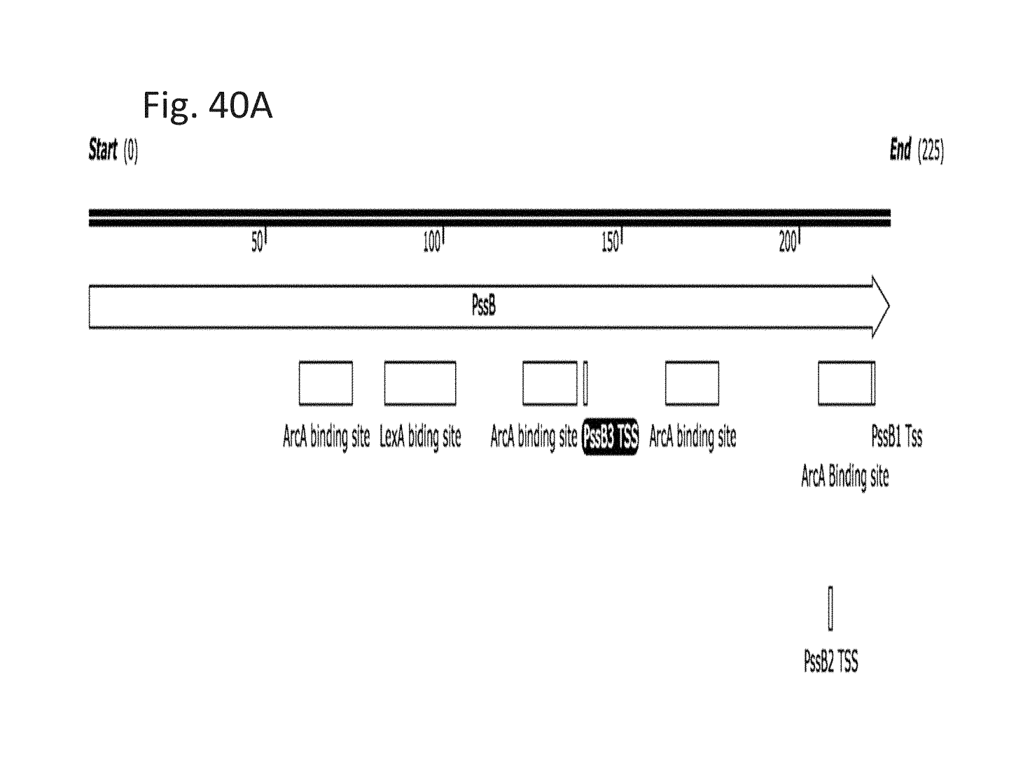

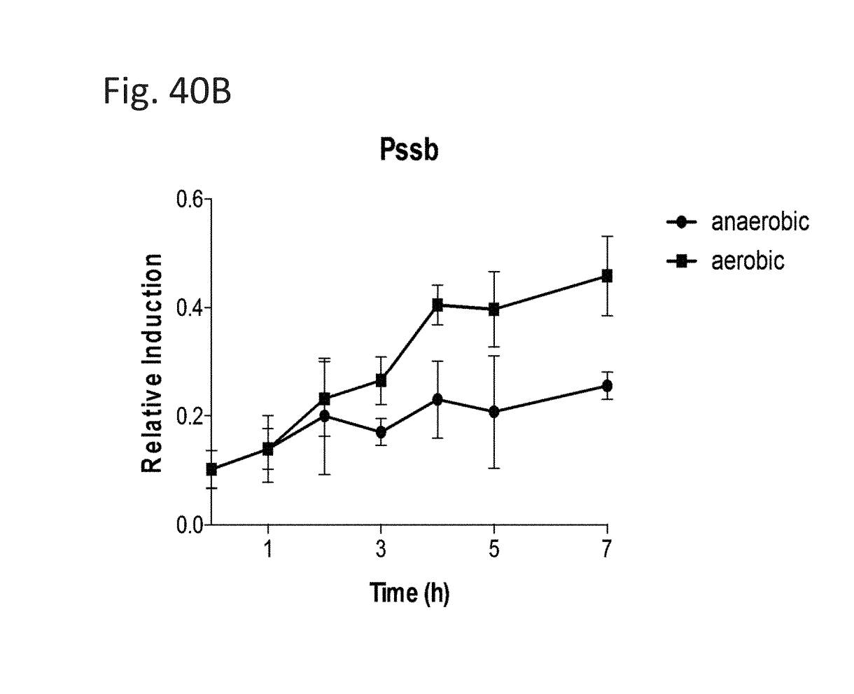

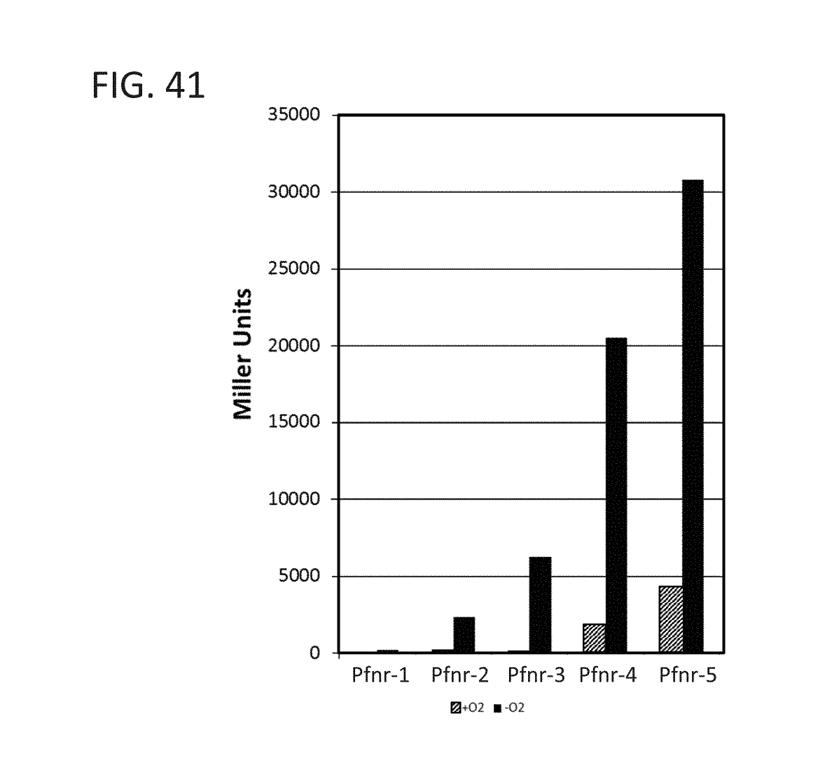

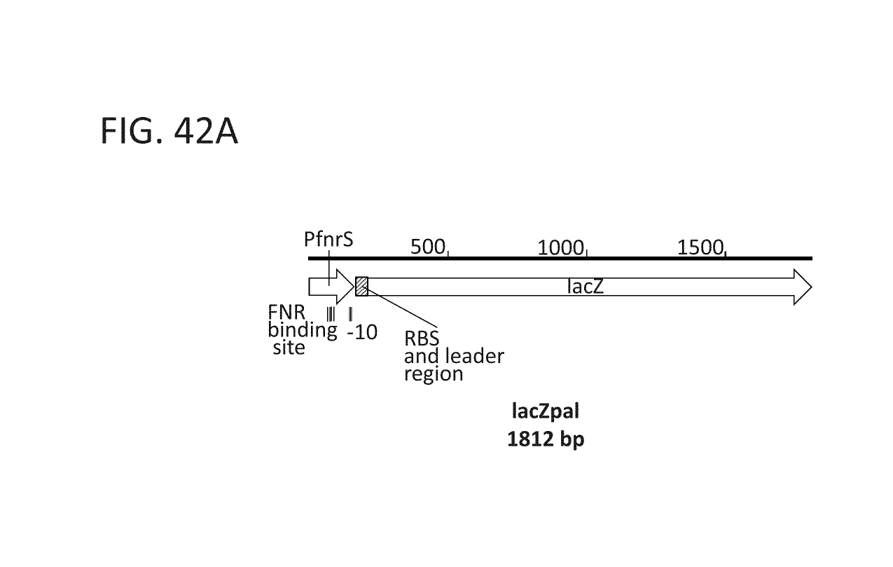

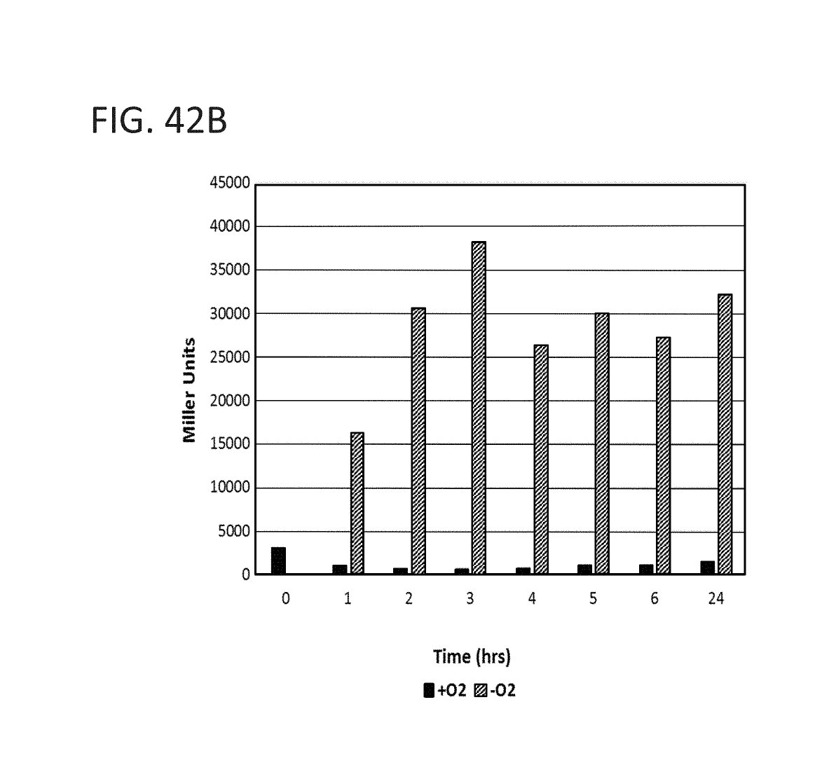

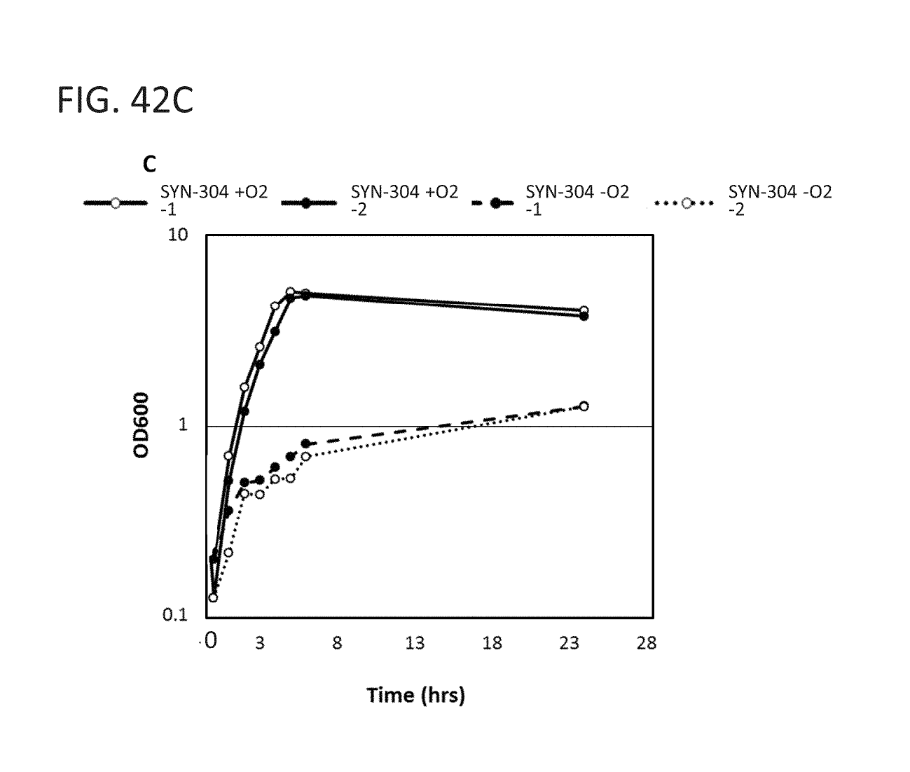

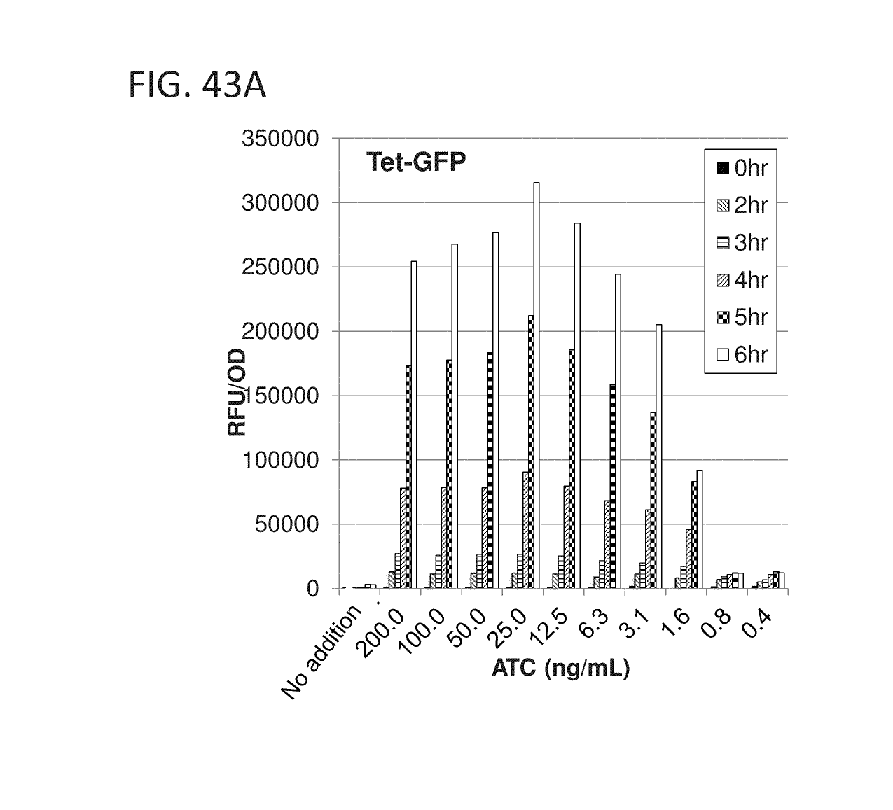

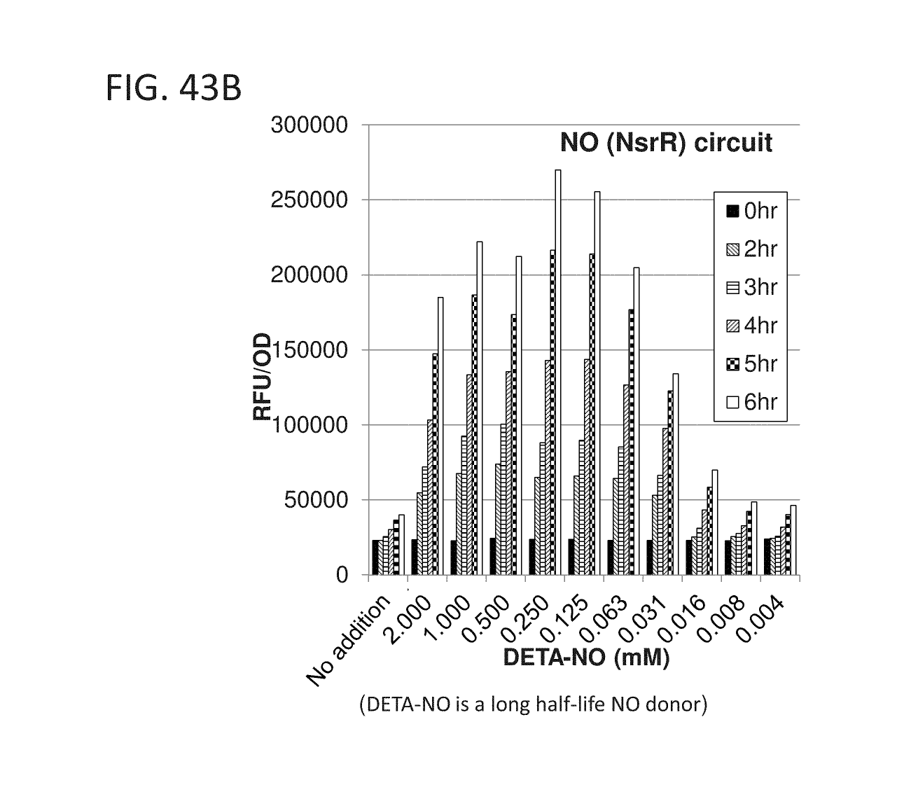

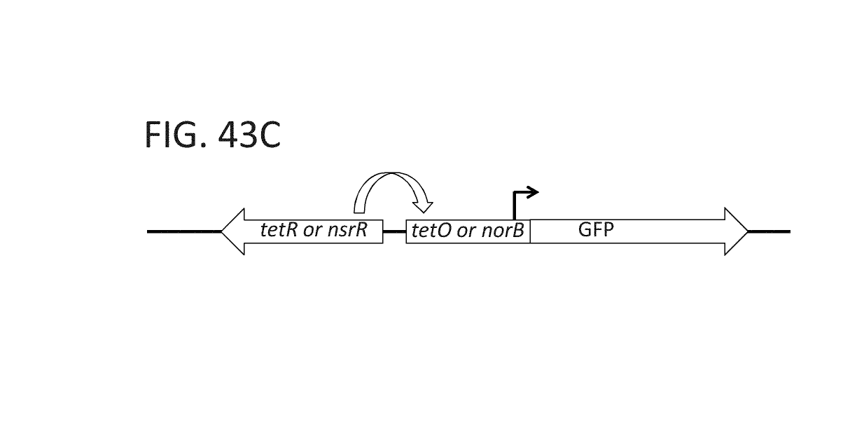

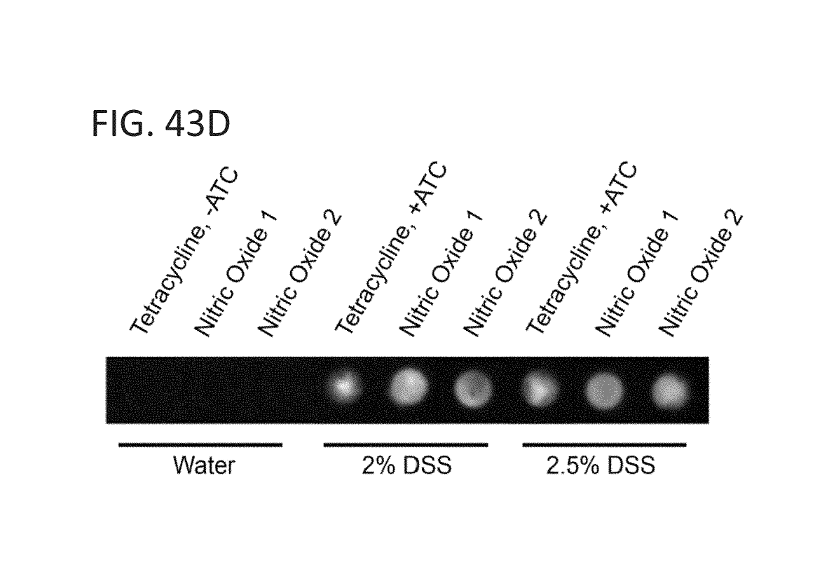

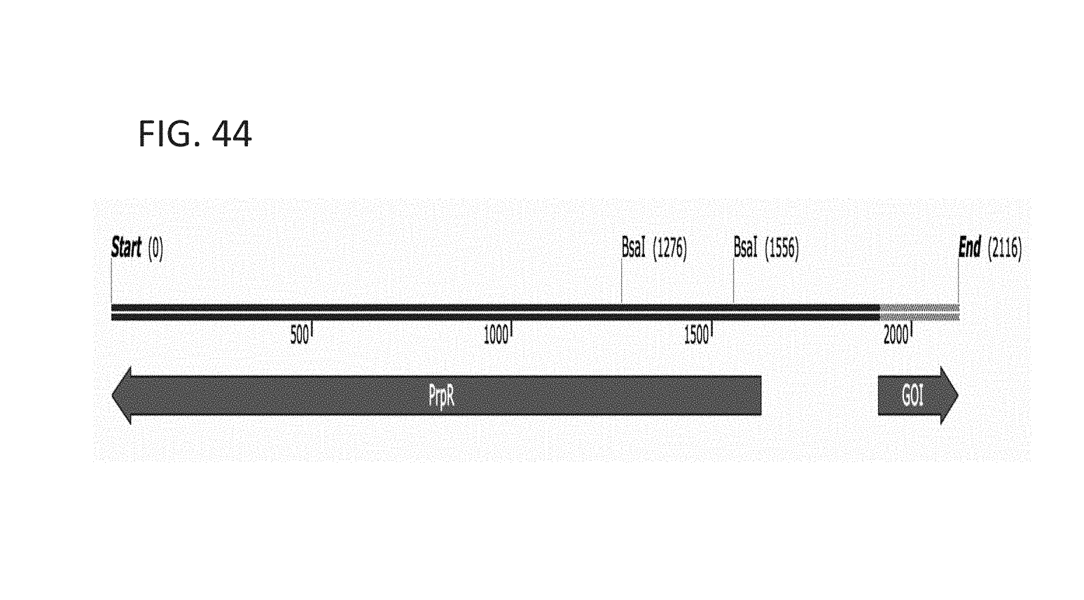

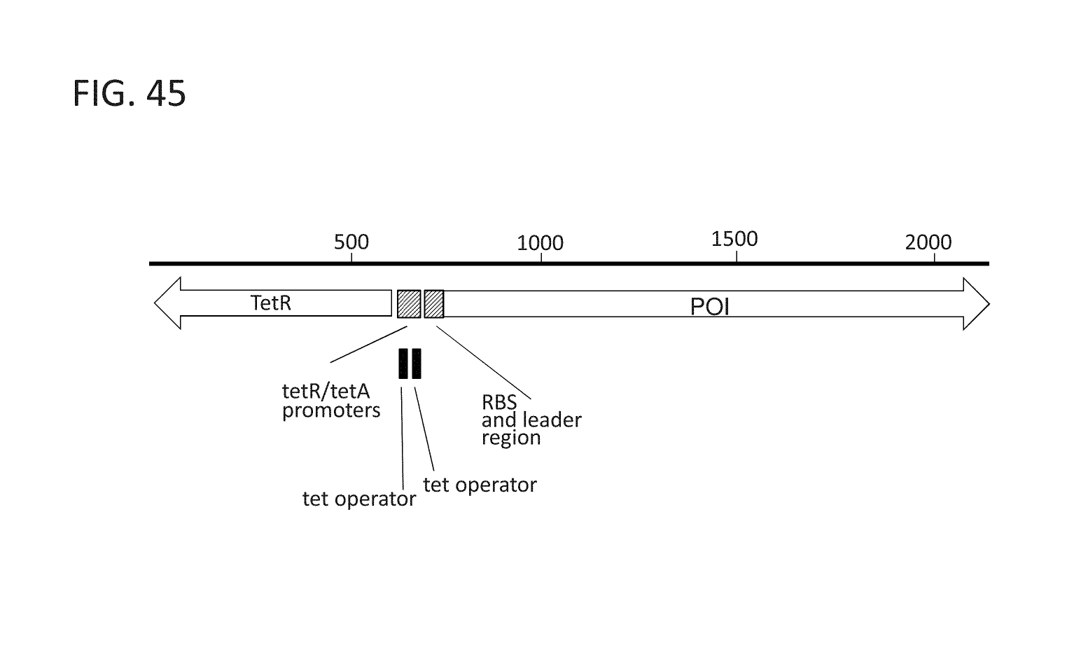

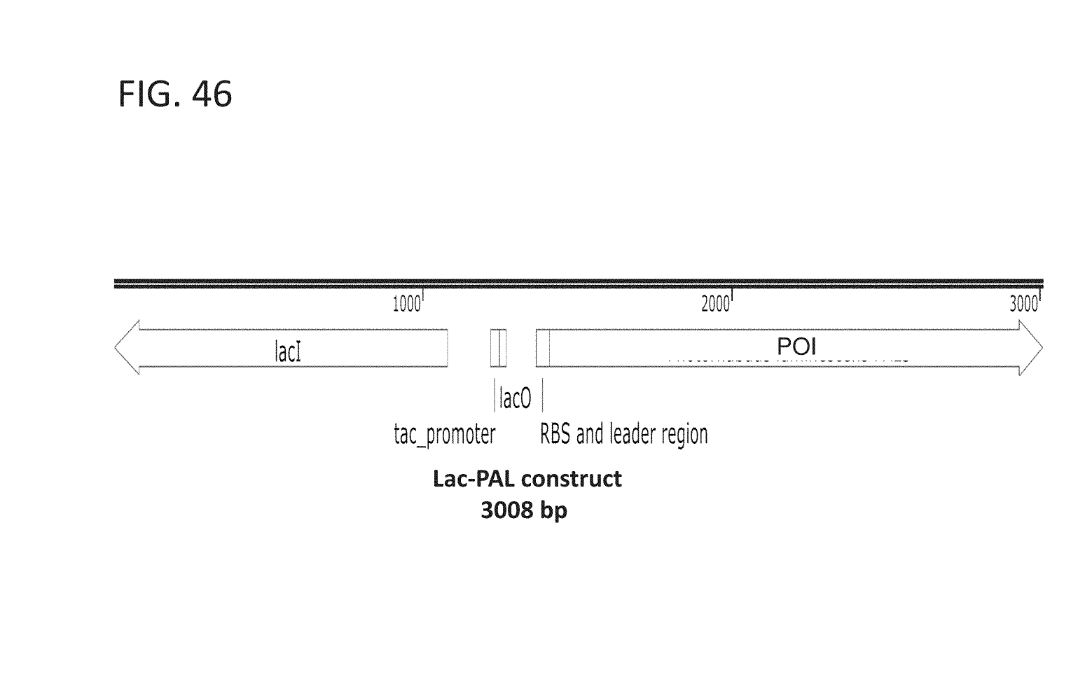

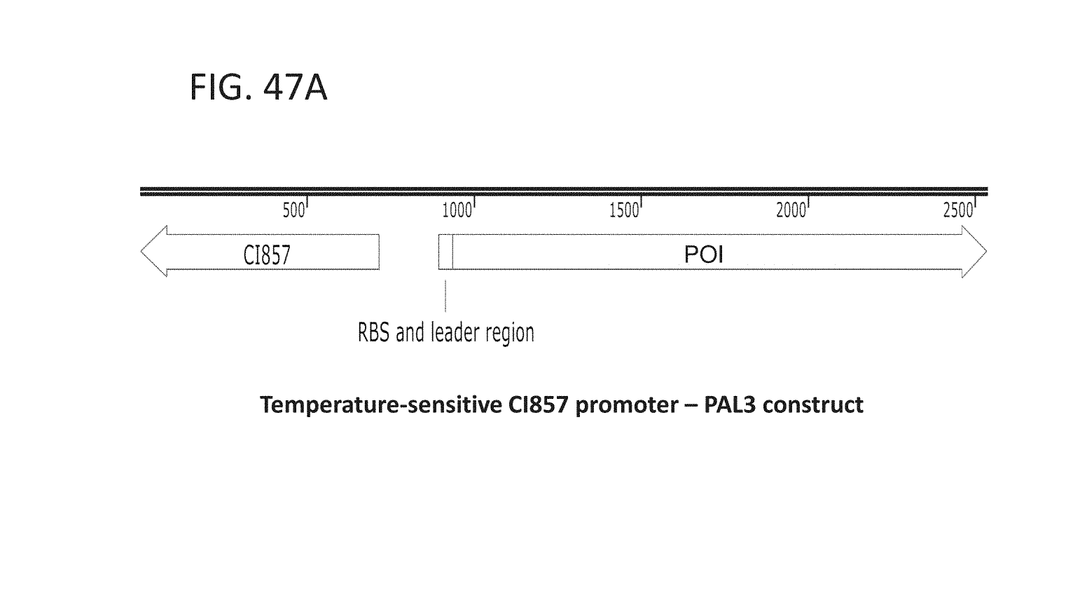

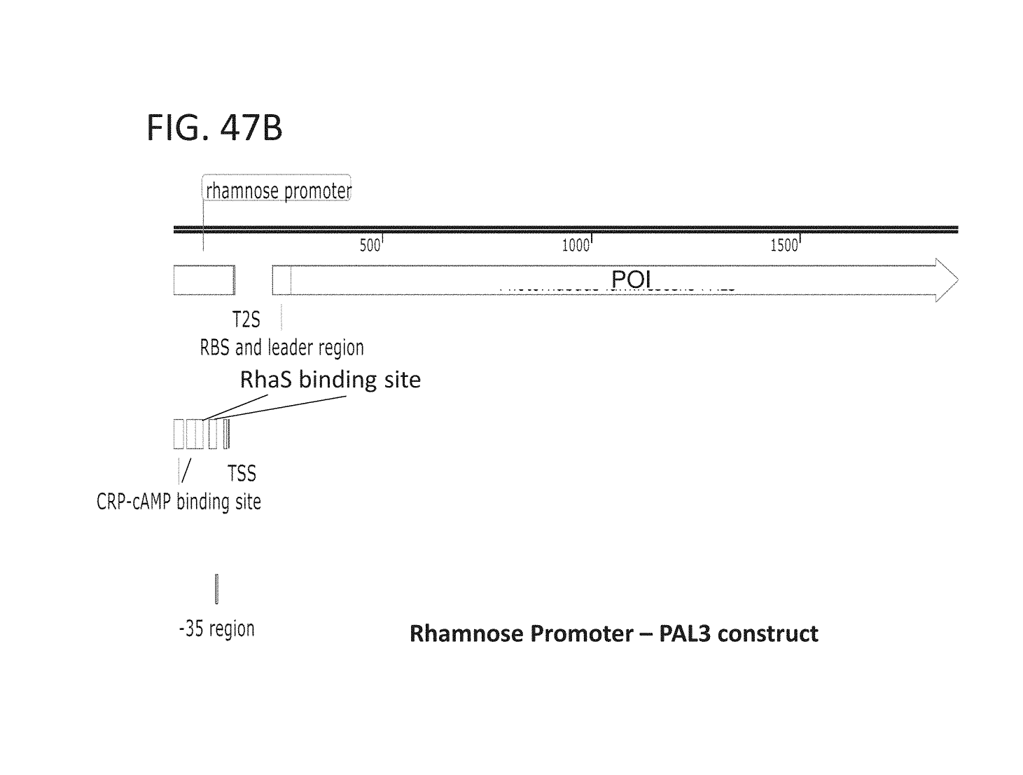

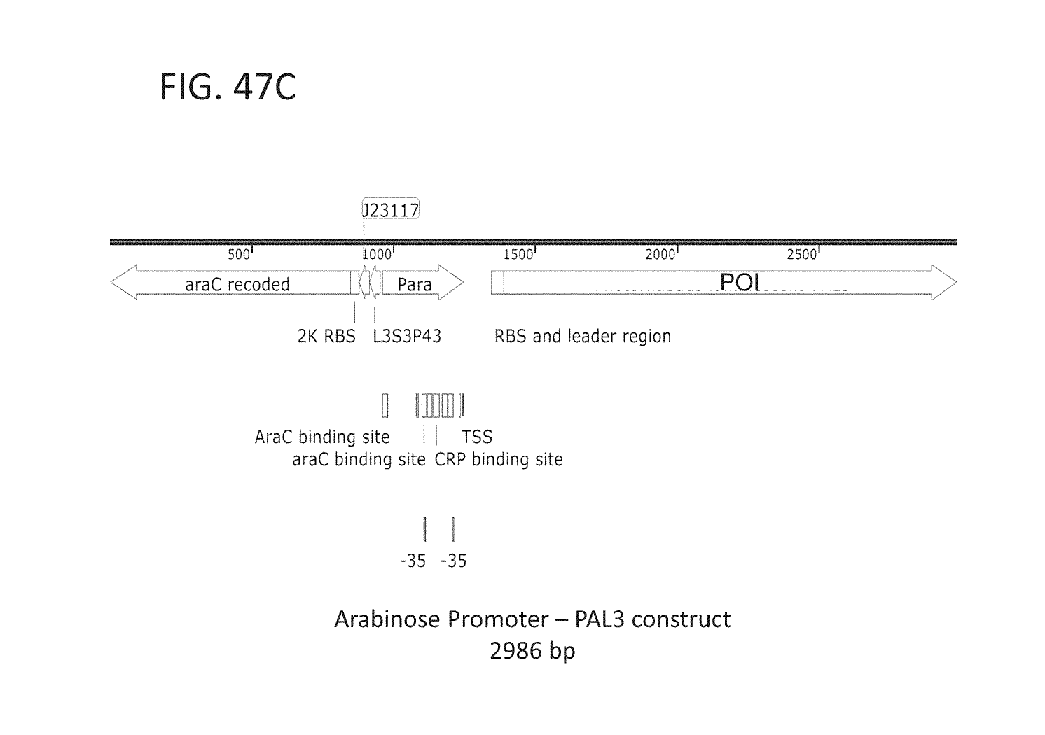

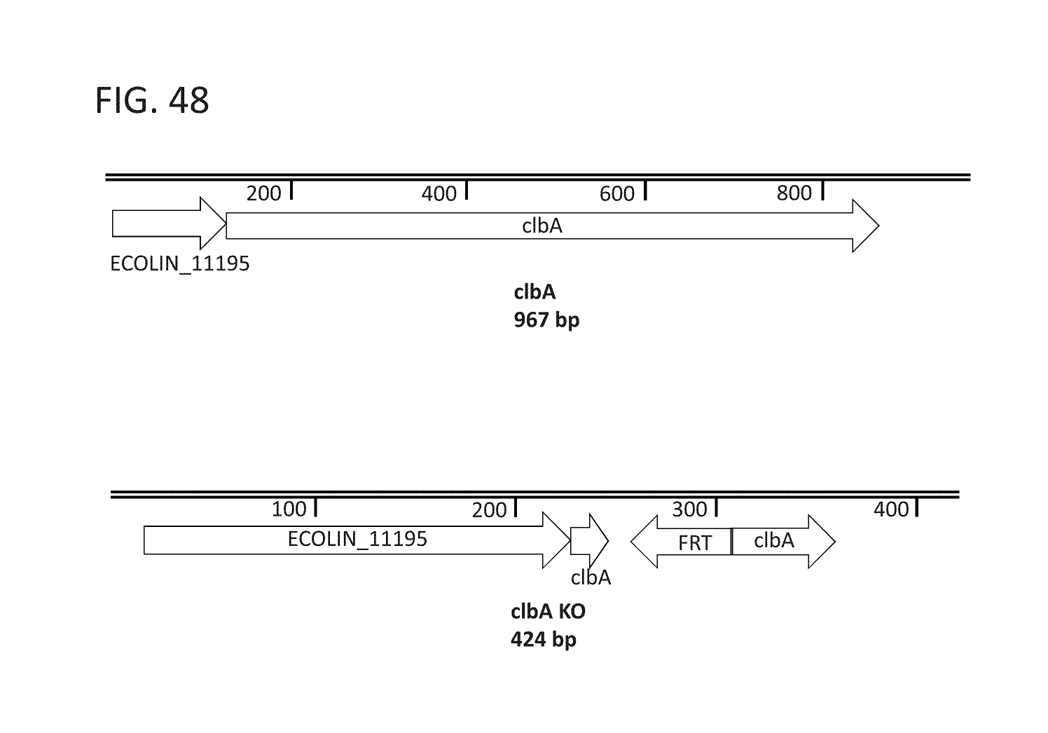

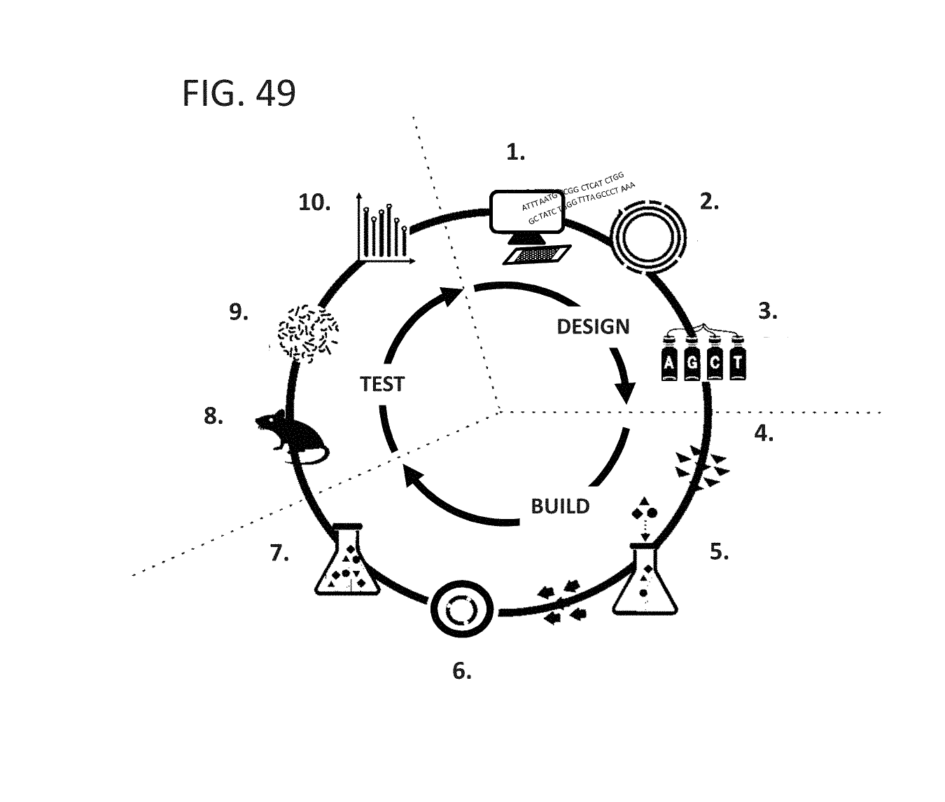

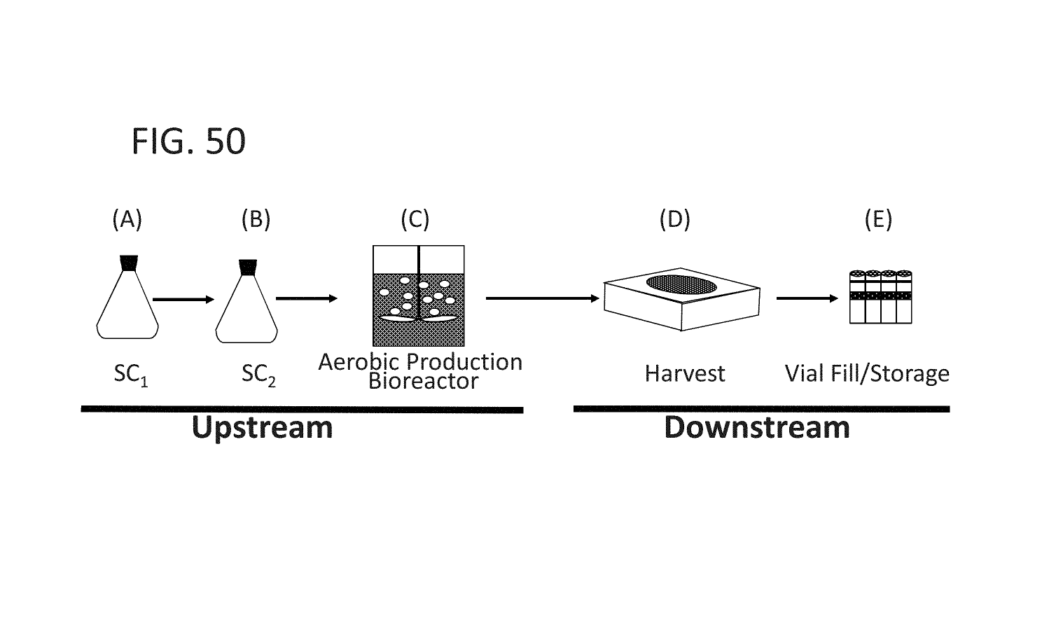

FIG. 39B depicts a strategy to allow the expression of one or more POI(s) under aerobic conditions through the arabinose inducible expression of FNRS24Y. By using a ribosome binding site optimization strategy, the levels of Fnr^{S24Y} expression can be fine-tuned, e.g., under optimal inducing conditions (adequate amounts of arabinose for full induction). Fine-tuning is accomplished by selection of an appropriate RBS with the appropriate translation initiation rate. Bioinformatics tools for optimization of RBS are known in the art.

FIG. 39C depicts a strategy to fine-tune the expression of a Para-POI construct by using a ribosome binding site optimization strategy. Bioinformatics tools for optimization of RBS are known in the art. In one strategy, arabinose controlled POI genes can be integrated into the chromosome to provide for efficient aerobic growth and pre-induction of the strain (e.g., in flasks, fermenters or other appropriate vesicles), while integrated versions of P_{fnrS}-POI constructs are maintained to allow for strong in vivo induction.

FIG. 40A depicts a schematic of the gene organization of a PssB promoter. The ssB gene product protects ssDNA from degradation; SSB interacts directly with numerous enzymes of DNA metabolism and is believed to have a central role in organizing the nucleoprotein complexes and processes involved in DNA replication (and replication restart), recombination and repair. The PssB promoter was cloned in front of a LacZ reporter and beta-galactosidase activity was measured. FIG. 40B depicts a bar graph showing the reporter gene activity for the PssB promoter under aerobic and anaerobic conditions. Briefly, cells were grown aerobically overnight, then diluted 1:100 and split into two different tubes. One tube was placed in the anaerobic chamber, and the other was kept in aerobic conditions for the length of the experiment. At specific times, the cells were analyzed for promoter induction. The PssB promoter is active under aerobic conditions, and shuts off under anaerobic conditions. This promoter can be used to express a gene of interest under aerobic conditions. This promoter can also be used to tightly control the expression of a gene product such that it is only expressed under anaerobic and/or low oxygen conditions. In this case, the oxygen induced PssB promoter induces the expression of a repressor, which represses the expression of a gene of interest. Thus, the gene of interest is only expressed in the absence of the repressor, i.e., under anaerobic and/or low oxygen conditions. This strategy has the advantage of an additional level of control for improved fine-tuning and tighter control. In one non-limiting example, this strategy can be used to control expression of thyA and/or dapA, e.g., to make a conditional auxotroph. The chromosomal copy of dapA or ThyA is knocked out. Under anaerobic and/or low oxygen conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic and/or low oxygen conditions, e.g., the gut, but prevent survival under aerobic conditions (biosafety switch).

FIG. 41 depicts β-galactosidase levels in samples comprising bacteria harboring a low-copy plasmid expressing lacZ from an FNR-responsive promoter selected from the exemplary FNR promoters. Different FNR-responsive promoters were used to create a library of anaerobic-inducible reporters with a variety of expression levels and dynamic ranges. These promoters included strong ribosome binding sites. Bacterial cultures were grown in either aerobic (+O2) or anaerobic conditions (—O2). Samples were removed at 4 hrs and the promoter activity based on β-galactosidase levels was analyzed by performing standard β-galactosidase colorimetric assays.

FIG. 42A depicts a schematic representation of the lacZ gene under the control of an exemplary FNR promoter (P_{fnrS}). LacZ encodes the β-galactosidase enzyme and is a common reporter gene in bacteria. FIG. 42B depicts FNR promoter activity as a function of β-galactosidase activity in SYN340. SYN340, an engineered bacterial strain harboring a low-copy fnrS-lacZ fusion gene, was grown in the presence or absence of oxygen. Values for standard β-galactosidase colorimetric assays are expressed in Miller units (Miller, 1972). These data suggest that the fnrS promoter begins to drive high-level gene expression within 1 hr under anaerobic conditions. FIG. 42C depicts the growth of bacterial cell cultures expressing lacZ over time, both in the presence and absence of oxygen.

FIG. 43A depicts ATC reporter constructs. FIG. 43B depicts nitric oxide-inducible reporter constructs. These constructs, when induced by their cognate inducer, lead to expression of GFP. Nissle cells harboring plasmids with either the control, ATC-inducible Pₜₑₜ-GFP reporter construct or the nitric oxide inducible P_{NsrR}-GFP reporter construct induced across a range of concentrations. Promoter activity is expressed as relative florescence units. FIG. 43C depicts a schematic of the constructs. FIG. 43D depicts a dot blot of bacteria harboring a plasmid expressing NsrR under control of a constitutive promoter and the reporter gene gfp (green fluorescent protein) under control of an NsrR-inducible promoter. DSS-treated mice serve as exemplary models for HE. As in HE subjects, the guts of mice are damaged by supplementing drinking water with 2-3% dextran sodium sulfate (DSS). Chemiluminescent is shown for NsrR-regulated promoters induced in DSS-treated mice.

FIG. 44 depicts the prpR propionate-responsive inducible promoter. The sequence for one propionate-responsive promoter is also disclosed herein as (SEQ ID NO:70).

FIG. 45 depicts the gene organization of an exemplary construct, comprising a cloned protein of interest (POI) gene under the control of a Tet promoter sequence and a Tet repressor gene.

FIG. 46 depicts the gene organization of an exemplary construct comprising Lad in reverse orientation, and a IPTG inducible promoter driving the expression of a protein of interest (POI, e.g., one or more metabolic effector(s) described herein). In some embodiments, this construct is useful for pre-induction and pre-loading of a therapeutic strain prior to in vivo administration under aerobic conditions and in the presence of inducer, e.g., IPTG. In some embodiments, this construct is used alone. In some embodiments, the construct is used in combination with other constitutive or inducible POI constructs, e.g., low oxygen, arabinose or IPTG inducible constructs. In some embodiments, the construct is used in combination with a low-oxygen inducible construct which is active in an in vivo setting.

FIG. 47A, FIG. 47B, and FIG. 47C depict schematics of non-limiting examples of constructs expressing a protein of interest (POI). FIG. 47A depicts a schematic of a non-limiting example of the organization of a construct for POI expression under the control a lambda CI inducible promoter. The construct also provides the coding sequence of a mutant of CI, CI857, which is a temperature sensitive mutant of CI. The temperature sensitive CI repressor mutant, CI857, binds tightly at 30 degrees C. but is unable to bind (repress) at temperatures of 37 C and above. In some embodiments, the construct comprises SEQ ID NO: 101. In some embodiments, this construct is used alone. In some embodiments, the temperature sensitive construct is used in combination with other constitutive or inducible POI constructs, e.g., low oxygen, arabinose, rhamnose, or IPTG inducible constructs. In some embodiments, the construct allows pre-induction and pre-loading of one or more POIs prior to in vivo administration. In some embodiments, the construct provides in vivo activity. In some embodiments, the construct is located on a plasmid, e.g., a low copy or a high copy plasmid. In some embodiments, the construct is located on a plasmid component of a biosafety system. In some embodiments, the construct is integrated into the bacterial chromosome at one or more locations. In some embodiments, the construct is used in combination with other POI constructs, which can either be provided on a plasmid or is integrated into the bacterial chromosome at one or more locations. In some embodiments, a temperature sensitive system can be used to set up a conditional auxotrophy. In a strain comprising deltaThyA or deltaDapA, a dapA or thyA gene can be introduced into the strain under the control of a thermoregulated promoter system. The strain can grow in the absence of Thy and Dap only at the permissive temperature, e.g., 37 C (and not lower).

FIG. 47B depicts a schematic of a non-limiting example of the organization of a construct for POI expression under the control of a rhamnose inducible promoter. For the application of the rhamnose expression system it is not necessary to express the regulatory proteins in larger quantities, because the amounts expressed from the chromosome are sufficient to activate transcription even on multi-copy plasmids. Therefore, only the rhaP BAD promoter is cloned upstream of the gene that is to be expressed. In some embodiments, this construct is used alone. In some embodiments, the rhamnose inducible construct is used in combination with other constitutive or inducible POI constructs, e.g., low oxygen, arabinose, temperature sensitive, or IPTG inducible constructs. In some embodiments, the construct allows pre-induction and pre-loading of one or more POIs prior to in vivo administration. In a non-limiting example, the construct is useful for pre-induction and is combined with low-oxygen inducible constructs. In some embodiments, the construct is located on a plasmid, e.g., a low copy or a high copy plasmid. In some embodiments, the construct is located on a plasmid component of a biosafety system. In some embodiments, the construct is integrated into the bacterial chromosome at one or more locations.

FIG. 47C depicts a schematic of a non-limiting example of the organization of a construct for POI expression under the control of an arabinose inducible promoter. The arabinose inducible POI construct comprises AraC (in reverse orientation), a region comprising an Arabinose inducible promoter, and the POI gene. In some embodiments, this construct is used alone. In some embodiments, the rhamnose inducible construct is used in combination with other constitutive or inducible POI constructs, e.g., low oxygen, arabinose, temperature sensitive, or IPTG inducible constructs. In some embodiments, the construct allows pre-induction and pre-loading of one or more POI(s) prior to in vivo administration. In a non-limiting example, the construct is useful for pre-induction and is combined with low-oxygen inducible constructs. In some embodiments, the construct is located on a plasmid, e.g., a low copy or a high copy plasmid. In some embodiments, the construct is located on a plasmid component of a biosafety system. In some embodiments, the construct is integrated into the bacterial chromosome at one or more locations.

FIG. 48 depicts a schematic of a wild-type clbA construct and a clbA knock-out construct.

FIG. 49 depicts a schematic of non-limiting processes for designing and producing the genetically engineered bacteria of the present disclosure. The step of “defining” comprises 1. Identification of diverse candidate approaches based on microbial physiology and disease biology; 2. Use of bioinformatics to determine candidate metabolic pathways; the use of prospective tools to determine performance targets required of optimized engineered synthetic biotics. The step of “designing” comprises the use of 1. Cutting-edge DNA assembly to enable combinatorial testing of pathway organization; 2. Mathematical models to predict pathway efficiency; 3. Internal stable of proprietary switches and parts to permit control and tuning of engineered circuits. The step of “Building” comprises 1. Building core structures “chassis,” 2. Stably integrating engineered circuits into optimal chromosomal locations for efficient expression; 3. Employing unique functional assays to assess genetic circuit fidelity and activity. The step of “integrating” comprises 1. Use of chromosomal markers, which enable monitoring of synthetic biotic localization and transit times in animal models; 2. Leveraging expert microbiome network and bioinformatics support to expand understanding of how specific disease states affect GI microbial flora and the behaviors of synthetic biotics in that environment; 3. Activating process development research and optimization in-house during the discovery phase, enabling rapid and seamless transition of development candidates to pre-clinical progression; Drawing upon extensive experience in specialized disease animal model refinement, which supports prudent, high quality testing of candidate synthetic biotics. Figure discloses SEQ ID NOs 316-317, respectively, in order of appearance.

FIG. 50A, FIG. 50B, FIG. 50C, FIG. 50D, and FIG. 50E depict a schematic of non-limiting manufacturing processes for upstream and downstream production of the genetically engineered bacteria of the present disclosure. FIG. 50A depicts the parameters for starter culture 1 (SC1): loop full—glycerol stock, duration overnight, temperature 37° C., shaking at 250 rpm. FIG. 50B depicts the parameters for starter culture 2 (SC2): 1/100 dilution from SC1, duration 1.5 hours, temperature 37° C., shaking at 250 rpm. FIG. 50C depicts the parameters for the production bioreactor: inoculum—SC2, temperature 37° C., pH set point 7.00, pH dead band 0.05, dissolved oxygen set point 50%, dissolved oxygen cascade agitation/gas FLO, agitation limits 300-1200 rpm, gas FLO limits 0.5-20 standard liters per minute, duration 24 hours. FIG. 50D depicts the parameters for harvest: centrifugation at speed 4000 rpm and duration 30 minutes, wash 1×10% glycerol/PBS, centrifugation, re-suspension 10% glycerol/PBS. FIG. 50E depicts the parameters for vial fill/storage: 1-2 mL aliquots, −80° C.

### DETAILED DESCRIPTION

The present disclosure provides engineered bacterial cells, pharmaceutical compositions thereof, and methods of modulating and treating disorders associated with propionate catabolism, such as propionic acidemia, methylmalonic acidemia, or vitamin B₁₂ deficiency. Specifically, the engineered bacteria disclosed herein have been constructed to comprise genetic circuits composed of, for example, at least one propionate catabolism enzyme. In some embodiments, the engineered bacteria additionally comprise optional circuitry to ensure the safety and non-colonization of the subject that is administered the engineered bacteria, such as auxotrophies, kill switches, etc. These engineered bacteria are safe and well tolerated and augment the innate activities of the subject's microbiome to achieve a therapeutic effect.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “engineered bacterial cell” or “engineered bacteria” refers to a bacterial cell or bacteria that have been genetically modified from their native state. For instance, an engineered bacterial cell may have nucleotide insertions, nucleotide deletions, nucleotide rearrangements, and/or nucleotide modifications introduced into their DNA. These genetic modifications may be present in the chromosome of the bacteria or bacterial cell, or on a plasmid in the bacteria or bacterial cell. Engineered bacterial cells disclosed herein may comprise exogenous nucleotide sequences on plasmids. Alternatively, engineered bacterial cells may comprise exogenous nucleotide sequences stably incorporated into their chromosome.

As used herein, the term “recombinant microorganism” refers to a microorganism, e.g., bacterial, yeast, or viral cell, or bacteria or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed microorganism” or “engineered microorganism” refers to a microorganism, e.g., bacterial, yeast, or viral cell, or bacteria or virus, that has been genetically modified from its native state to perform a specific function, e.g., to metabolize propionate and/or one or more of its metabolites. In certain embodiments, the programmed or engineered microorganism has been modified to express one or more proteins, for example, one or more proteins that have a therapeutic activity or serve a therapeutic purpose. The programmed or engineered microorganism may additionally have the ability to stop growing or to destroy itself once the protein(s) of interest have been expressed.

A “programmed bacterial cell” or “engineered bacterial cell” is a bacterial cell that has been genetically modified from its native state. In certain embodiments, the programmed or engineered bacterial cell has been modified from its native state to perform a specific function, for example, to express one or more proteins, for example, one or more proteins that have a therapeutic activity or serve a therapeutic purpose, e.g., to metabolize a propionate and/or one or more of its metabolites. The programmed or engineered bacterial cell may additionally have the ability to stop growing or to destroy itself once the protein(s) of interest have been expressed. For instance, an engineered bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Engineered bacterial cells disclosed herein may comprise exogenous nucleotide sequences on plasmids. Alternatively, engineered bacterial cells may comprise exogenous nucleotide sequences stably incorporated into their chromosome.

As used herein, the term “gene” refers to any nucleic acid sequence that encodes a polypeptide, protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature. The term “gene” is meant to encompass full-length gene sequences (e.g., as found in nature and/or a gene sequence encoding a full-length polypeptide or protein) and is also meant to include partial gene sequences (e.g., a fragment of the gene sequence found in nature and/or a gene sequence encoding a protein or fragment of a polypeptide or protein). The term “gene” is meant to encompass modified gene sequences (e.g., modified as compared to the sequence found in nature). Thus, the term “gene” is not limited to the natural or full-length gene sequence found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a “heterologous sequence” encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene that is operably linked to a promoter that is not associated with said gene in nature. For example, in some embodiments, the genetically engineered bacteria disclosed herein comprise a gene that is operably linked to a directly or indirectly inducible promoter that is not associated with said gene in nature, e.g., an FNR responsive promoter (or other promoter disclosed herein) operably linked to a gene encoding a propionate catabolism enzyme. In some embodiments, the genetically engineered virus of the disclosure comprises a gene that is operably linked to a directly or indirectly inducible promoter that is not associated with said gene in nature, e.g., a promoter operably linked to a gene encoding a propionate catabolism enzyme.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or other promoter disclosed herein.

As used herein, “stably maintained” or “stable” bacterium is used to refer to a bacterial host cell carrying non-native genetic material, e.g., a gene encoding a propionate catabolism enzyme, which is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene encoding a propionate catabolism enzyme, in which the plasmid or chromosome carrying the gene is stably maintained in the bacterium, such that propionate catabolism enzyme can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, a “gene cassette” or “operon” encoding a propionate catabolism pathway refers to the two or more genes that are required to catabolize propionate, propionyl CoA, methylmalonic acid, or methylmalonyl CoA into an inert end-product, e.g., succinate or polyhydroxyalkanoates. In addition to encoding a set of genes capable of producing said molecule, the gene cassette or operon may also comprise additional transcription and translation elements, e.g., a ribosome binding site. Each gene or gene cassette may be present on a plasmid or bacterial chromosome. In addition, multiple copies of any gene, gene cassette, or regulatory region may be present in the bacterium, wherein one or more copies of the gene, gene cassette, or regulatory region may be mutated or otherwise altered as described herein. In some embodiments, the genetically engineered bacteria are engineered to comprise multiple copies of the same gene, gene cassette, or regulatory region in order to enhance copy number or to comprise multiple different components of a gene cassette performing multiple different functions.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding a propionate catabolism enzyme, that is joined to a regulatory sequence in a manner which allows expression of the nucleic acid sequence, e.g., the gene encoding the propionate catabolism enzyme. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein. Examples of other inducible promoters are provided herein below.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid may comprise a nucleic acid sequence encoding one or more heterologous gene(s) or gene cassette(s).

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising a propionate catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

Specifically, the term “genetic modification that increases import of propionate into the bacterial cell” refers to a genetic modification that increases the uptake rate or increases the uptake quantity of propionate, propionyl CoA, methylmalonic acid, or methylmalonyl CoA or metabolites thereof, into the cytosol of the bacterial cell, as compared to the uptake rate or uptake quantity of the propionate, propionyl CoA, methylmalonic acid, or methylmalonyl CoA into the cytosol of a bacterial cell not having said modification, e.g., a wild-type bacterial cell. In one embodiment, an engineered bacterial cell having a genetic modification that increases import of propionate into the bacterial cell refers to a bacterial cell comprising a heterologous gene encoding a transporter of propionate. In one embodiment, a recombinant bacterial cell having a genetic modification that increases import of propionate, propionyl CoA, methylmalonic acid, or methylmalonyl CoA and/or their metabolites from the bacterial cell comprises a genetic mutation in a native gene. In another embodiment, a recombinant bacterial cell having a genetic modification that increases import of a propionate and/or its metabolites from the bacterial cell comprises a genetic mutation in a native promoter, which increases or activates transcription of the gene which increases import of propionate, propionyl CoA, methylmalonic acid, or methylmalonyl CoA and/or their metabolites. In another embodiment, a recombinant bacterial cell having a genetic modification that increases import of p propionate, propionyl CoA, methylmalonic acid, or methylmalonyl CoA and/or their metabolites from the bacterial cell comprises a genetic mutation leading to overexpression of an activator of an importer (transporter) of propionate and/or its metabolites. In another embodiment, a recombinant bacterial cell having a genetic modification that increases import of propionate, propionyl CoA, methylmalonic acid, or methylmalonyl CoA and/or their metabolites from the bacterial cell comprises a genetic mutation which increases or activates translation of the gene encoding the transporter (importer).

Moreover, the term “genetic modification that increases import of a propionate and/or its metabolites into the bacterial cell” refers to a genetic modification that increases the uptake rate or increases the uptake quantity of a propionate, propionyl CoA, methylmalonic acid, or methylmalonyl CoA and/or their metabolites into the cytosol of the bacterial cell, as compared to the uptake rate or uptake quantity of propionate and/or its metabolites into the cytosol of a bacterial cell not having said modification, e.g., a wild-type bacterial cell. In some embodiments, an engineered bacterial cell having a genetic modification that increases import of propionate, propionyl CoA, methylmalonic acid, or methylmalonyl CoA and/or their metabolites into the bacterial cell refers to a bacterial cell comprising heterologous gene sequence (native or non-native) encoding one or more importer(s) (transporter(s)) of propionate, propionyl CoA, methylmalonic acid, or methylmalonyl CoA and/or their metabolites. In some embodiments, the genetically engineered bacteria comprising genetic modification that increases import of propionate and one or more of its metabolites into the bacterial cell comprise gene sequence(s) encoding a propionate transporter or other amino acid transporter that transports one or more propionate metabolites into the bacterial cell, for example a transporter that is capable of transporting methylmalonic acid into a bacterial cell. The transporter can be any transporter that assists or allows import of propionate and/or metabolites thereof into the cell. In certain embodiments, the propionate transporter is one of MctC, PutP_6, or any other propionate transporters described herein. In certain embodiments, the engineered bacterial cell contains gene sequences encoding MctC, PutP_6, or any other propionate transporters described herein. In some embodiments, the engineered bacteria comprise more than one copy of gene sequence encoding a propionate transporter. In some embodiments, the engineered bacteria comprise gene sequence(s) encoding more than one propionate transporter, e.g., two or more different propionate transporters.

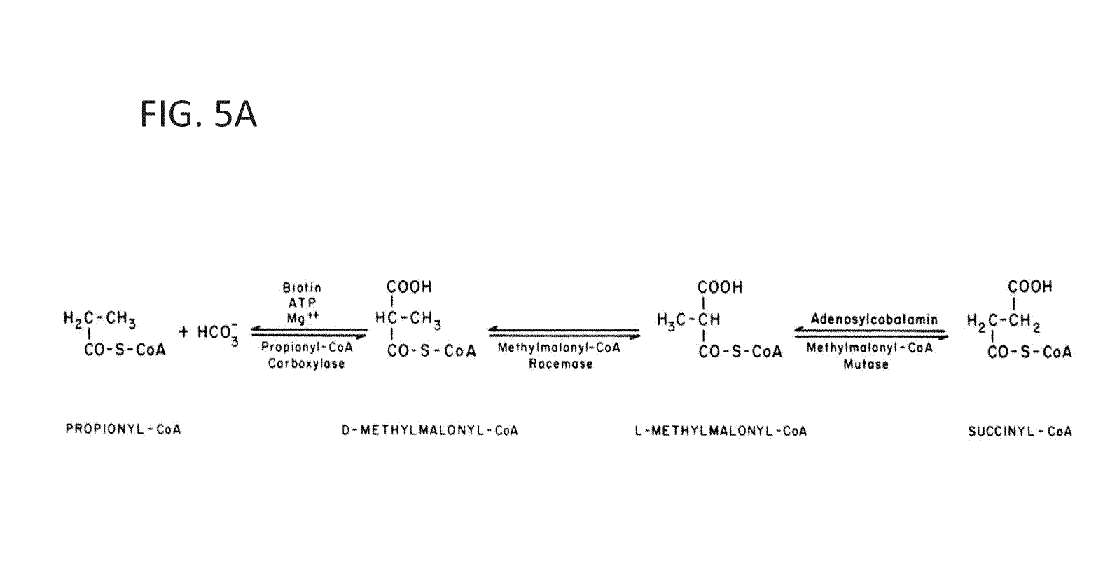

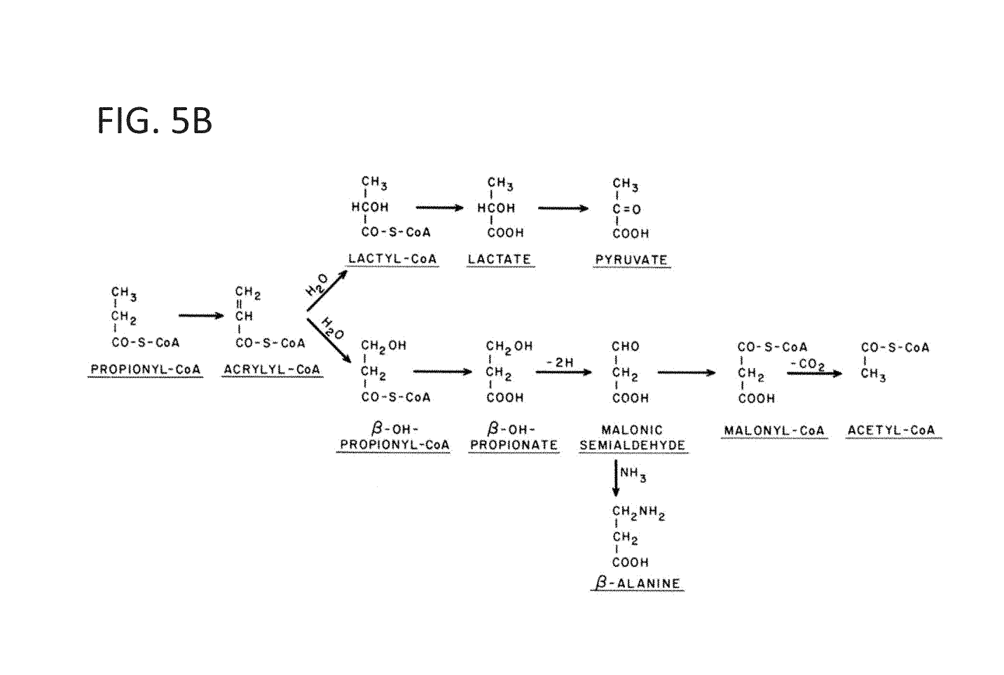

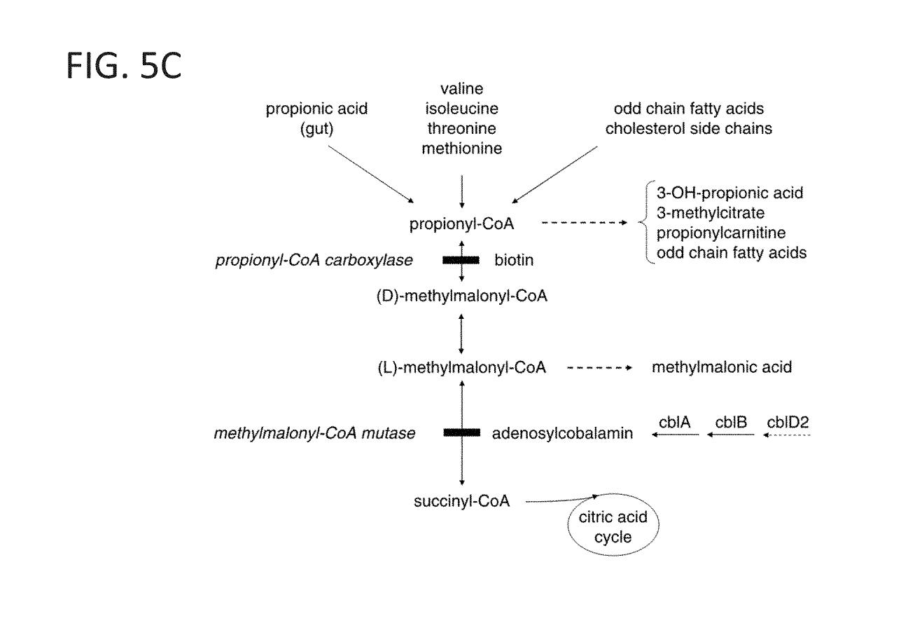

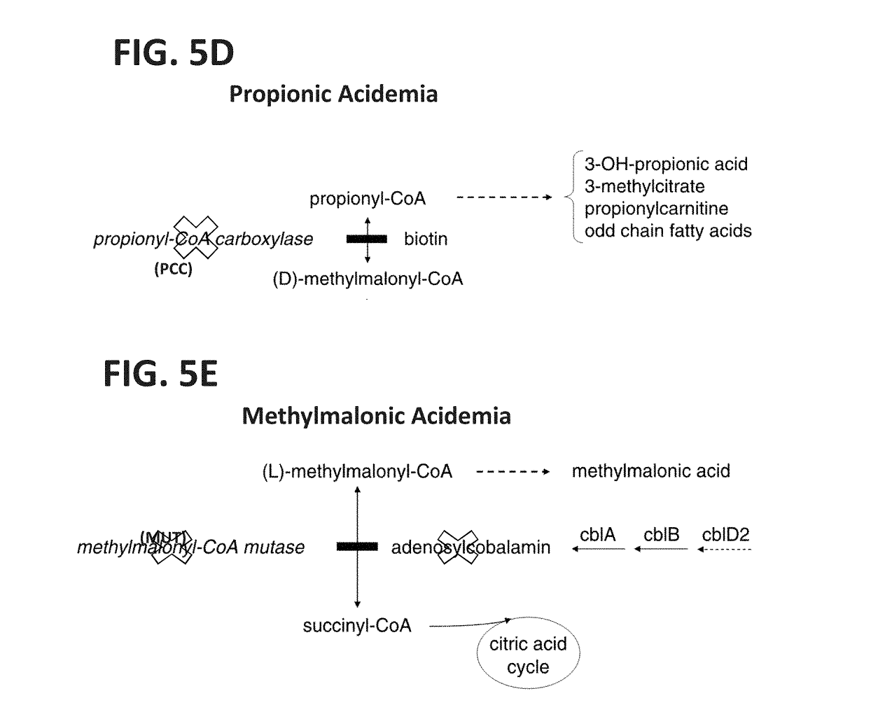

The term “propionate,” as used herein, refers to C2H5COO—. Propionate is the conjugate base of propionic acid. The term “propionic acid,” as used herein, refers to a carboxylic acid with the chemical formula CH3CH2COOH. Propionate is converted to propionyl coenzyme A (“propionyl CoA”) as a first step in the catabolism of carboxylic acids. Propionate and propionyl CoA exist in an equilibrium. In humans and other vertebrates, propionyl CoA is carboxylated to D-methylmalonyl CoA by the enzyme Propionyl CoA Carboxylase (PCC) with the help of biotin (vitamin B7), which is isomerized to L-methylmalonyl CoA (see FIG. 5). As used herein, the term “methylmalonyl CoA” refers to the thioester consisting of coenzyme A linked to methylmalonic acid. A vitamin B12-dependent enzyme, Methylmalonyl CoA Mutase (MUT) then catalyzes the rearrangement of L-methylmalonyl CoA to succinyl CoA, which is then incorporated into the citric acid cycle.

As used herein, the term “propionate binding protein” refers to a protein which can bind to propionate and/or one or more propionate metabolites, including, but not limited to, methylmalonate and/or methylmalonic acid.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu.

As used herein, the term “propionate transporter” refers to a polypeptide which functions to transport propionate and/or one or more of its metabolites, including, but not limited to, methylmalonate and/or methylmalonic acid into the bacterial cell.

As used herein, the term “polypeptide of interest” or “polypeptides of interest”, “protein of interest”, “proteins of interest”, “payload”, “payloads” includes any or a plurality of any of the propionate catabolism enzymes, propionate and/or methylmalonate importers and/or succinate exporters described herein. As used herein, the term “gene of interest” or “gene sequence of interest” includes any or a plurality of any of the gene(s) an/or gene sequence(s) and or gene cassette(s) encoding one or more propionate catabolism enzymes, propionate and/or methylmalonate importers and/or succinate exporters described herein.

As used herein the terms “methylmalonic acid” and “methylmalonate” are used interchangeably. As used herein, the terms “propionate” and “propionic acid” are used interchangeably.

As used herein, the phrase “propionate and/or its metabolites” or “propionate and/or one or more of its metabolites”, includes any metabolite of propionate, such as any of the metabolites described herein, and also includes propionyl CoA, methylmalonic acid, or methylmalonyl CoA.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Non-pathogenic bacteria” refer to bacteria that are not capable of causing disease or harmful responses in a host. In some embodiments, non-pathogenic bacteria are commensal bacteria. Examples of non-pathogenic bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. No. 6,835,376; U.S. Pat. No. 6,203,797; U.S. Pat. No. 5,589,168; U.S. Pat. No. 7,731,976). Naturally pathogenic bacteria may be genetically engineered to provide reduce or eliminate pathogenicity.

As used herein, the term “treat” and its cognates refer to an amelioration of a disease, or at least one discernible symptom thereof. In another embodiment, “treat” refers to an amelioration of at least one measurable physical parameter, not necessarily discernible by the patient. In another embodiment, “treat” refers to inhibiting the progression of a disease, either physically (e.g., stabilization of a discernible symptom), physiologically (e.g., stabilization of a physical parameter), or both. In another embodiment, “treat” refers to slowing the progression or reversing the progression of a disease. As used herein, “prevent” and its cognates refer to delaying the onset or reducing the risk of acquiring a given disease.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Diseases associated with the catabolism of propionate, e.g., Propionic Acidemia (PA) or Methylmalonic Acidemia (MMA), may be caused by inborn genetic mutations for which there are no known cures. Diseases can also be secondary to other conditions, e.g., liver diseases. Treating diseases involving the catabolism of propionate, such as PA or MMA, may encompass reducing normal levels of propionate, propionic acid, propionyl CoA, methylmalonic acid, and/or methylmalonyl CoA, reducing excess levels of propionate, propionic acid, propionyl CoA, methylmalonic acid, and/or methylmalonyl CoA, or eliminating propionate, propionic acid, propionyl CoA, methylmalonic acid, and/or methylmalonyl CoA, and does not necessarily encompass the elimination of the underlying disease.

As used herein, the term “catabolism” refers to the conversion of an odd-chain fatty acid, cholesterol, or branched chain amino acid, such as methionine, threonine, isoleucine, or valine, into its corresponding propionyl CoA, methylmalonyl CoA, or succinyl CoA. In one embodiment, “abnormal catabolism” refers to a decrease in the rate or the level of conversion of an odd-chain fatty acid, cholesterol, or branched chain amino acid into its corresponding propionyl CoA, methylmalonyl CoA, or succinyl CoA, leading to the build-up of propionyl CoA or methylmalonyl CoA in the blood or the brain of a subject. In one embodiment, build-up of propionyl CoA or methylmalonyl CoA in the blood or the brain of a subject becomes toxic and leads to the development of a disease or disorder associated with the abnormal catabolism of propionate in the subject. “Catabolism” e.g., “Propionate catabolism”, also refers to the breakdown of propionate and/or methylmalonic acid to one or more of its breakdown products as described herein.

In one embodiment, a “disorder involving the catabolism of propionate” is a disease or disorder involving the abnormal catabolism of propionate, propionyl CoA, methylmalonic acid, or methylmalonyl CoA. As used herein, the term “disorder involving the abnormal catabolism of propionate” refers to a disease or disorder wherein the catabolism of propionate, propionyl CoA, methylmalonic acid, and/or methylmalonyl CoA is abnormal. In one embodiment, “abnormal catabolism” refers to a decrease in the rate or the level of conversion of propionyl CoA into methylmalonyl CoA, or a decrease in the rate or the level of conversion of methylmalonyl CoA into succinyl CoA, leading to the build-up of propionate, propionyl CoA, methylmalonic acid, and/or methylmalonyl CoA in the blood or the brain of a subject. In one embodiment, build-up of the propionate, propionyl CoA, methylmalonic acid, and/or methylmalonyl CoA in the blood or the brain of a subject becomes toxic and leads to the development of a disease or disorder associated with the abnormal catabolism of propionate in the subject. In one embodiment, the disorder involving the abnormal catabolism of propionate is Propionic Acidemia or Methylmalonic Acidemia.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to a propionate catabolism enzyme disease, e.g., Propionic Acidemia and/or Methylmalonic Acidemia. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 1
Examples of transcription factors and responsive
genes and regulatory regions

| Transcription | Examples of responsive genes, |
|---|---|
| Factor | promoters, and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

In some embodiments, the exogenous environmental conditions are the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

As used herein, the term “low oxygen” is meant to refer to a level, amount, or concentration of oxygen (O₂) that is lower than the level, amount, or concentration of oxygen that is present in the atmosphere (e.g., <21% O₂, <160 torr O₂)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 2 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%, 0.44%, 0.3%, 0.25%, 0.2%, 0.1%, 0.08%, 0.075%, 0.058%, 0.04%, 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%, 0.44%, 0.3%, 0.25%, 0.2%, 0.1%, 0.08%, 0.075%, 0.058%, 0.04%, 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 2

| Compartment | Oxygen Tension |
|---|---|
| stomach | ~60 torr (e.g., 58 +/− 15 torr) |
| duodenum and first | ~30 torr (e.g., 32 +/− 8 torr); ~20% oxygen in |
| part of jejunum | ambient air |
| Ileum (mid-small | ~10 torr; ~6% oxygen in ambient air (e.g., 11 +/− 3 |
| intestine) | torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, viruses, parasites, fungi, certain algae, yeast, and protozoa. In some aspects, the microorganism is engineered (“engineered microorganism”) to produce one or more therapeutic molecules, e.g., lysosomal enzyme(s). In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. No. 6,835,376; U.S. Pat. No. 6,203,797; U.S. Pat. No. 5,589,168; U.S. Pat. No. 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia Coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. No. 5,589,168; U.S. Pat. No. 6,203,797; U.S. Pat. No. 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Diseases associated with the catabolism of propionate and/or one or more of its metabolites, e.g., Propionic Acidemia and/or Methylmalonic Acidemia, may be caused by inborn genetic mutations for which there are no known cures. Diseases can also be secondary to other conditions. Treating diseases involving the catabolism of propionate and methylmalonate, e.g., Propionic Acidemia and/or Methylmalonic Acidemia, may encompass reducing normal levels of propionate and/or one or more of its metabolites, reducing excess levels of propionate and/or one or more of its metabolites, or eliminating of propionate and/or one or more of its metabolites and does not necessarily encompass the elimination of the underlying disease.

As used herein, “payload” refers to one or more molecules of interest to be produced by a genetically engineered microorganism, such as bacterium or a virus. In some embodiments, the payload is a therapeutic payload, e.g., a propionate catabolic enzyme or a propionate transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments, the payload comprises a repressor element, such as a kill switch. In some embodiments, the payload comprises an antibiotic resistance gene or genes. In some embodiments, the payload is encoded by a gene, multiple genes, gene cassette, or an operon. In alternate embodiments, the payload is produced by a biosynthetic or biochemical pathway, wherein the biosynthetic or biochemical pathway may optionally be endogenous to the microorganism. In alternate embodiments, the payload is produced by a biosynthetic or biochemical pathway, wherein the biosynthetic or biochemical pathway is not endogenous to the microorganism. In some embodiments, the genetically engineered microorganism comprises two or more payloads.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting a biomolecule, e.g., polypeptide from the microbial, e.g., bacterial cytoplasm. The secretion system may comprise a single protein or may comprise two or more proteins assembled in a complex e.g. HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the polypeptide to be secreted include a “secretion tag” of either RNA or peptide origin to direct the polypeptide to specific secretion systems. In some embodiments, the secretion system is able to remove this tag before secreting the polypeptide from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the lysosomal enzyme(s) into the extracellular milieu. In some embodiments, the secretion system involves the generation of a “leaky” or de-stabilized outer membrane, which may be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases, such as degS, degP or nlpl, are deactivated. Thus, in some embodiments, the engineered bacteria have one or more deleted or mutated membrane genes, e.g., selected from lpp, ompA, ompA, ompF, tolA, tolB, and pal genes. In some embodiments, the engineered bacteria have one or more deleted or mutated periplasmic protease genes, e.g., selected from degS, degP, and nlpl. In some embodiments, the engineered bacteria have one or more deleted or mutated gene(s), selected from lpp, ompA, ompA, ompF, tolA, tolB, pal, degS, degP, and nlpl genes.

As used herein a “pharmaceutical composition” refers to a preparation of bacterial cells with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a disease. A therapeutically effective amount may, for example, be sufficient to treat, prevent, reduce the severity, delay the onset, and/or reduce the risk of occurrence of one or more symptoms of the disease. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of engineered bacterial cell of the disclosure.

As used herein, the term “bactericidal” refers to a molecule or protein which is capable of killing the engineered bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the engineered bacterial cell of the disclosure, or which is capable of killing the engineered bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of or” one or more of the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Bacterial Strains

The disclosure provides a bacterial cell that comprises at least one heterologous gene encoding a propionate catabolism enzyme. In some embodiments, the bacterial cell is a non-pathogenic bacterial cell. In some embodiments, the bacterial cell is a commensal bacterial cell. In some embodiments, the bacterial cell is a probiotic bacterial cell.

In certain embodiments, the bacterial cell is selected from the group consisting of a Bacteroides fragilis, Bacteroides thetaiotaomicron, Bacteroides subtilis, Bifidobacterium animalis, Bifidobacterium bifidum, Bifidobacterium infantis, Bifidobacterium lactis, Clostridium butyricum, Clostridium scindens, Escherichia coli, Lactobacillus acidophilus, Lactobacillus plantarum, Lactobacillus reuteri, Lactococcus lactis, and Oxalobacter formigenes bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium animalis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is a Clostridium scindens bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a Oxalobacter formigenes bacterial cell. In another embodiment, the bacterial cell does not include Oxalobacter formigenes.

In one embodiment, the bacterial cell is a Gram positive bacterial cell. In another embodiment, the bacterial cell is a Gram negative bacterial cell.

In some embodiments, the bacterial cell is Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008), and E. coli Nissle does not carry pathogenic adhesion factors and does not produce any enterotoxins or cytotoxins, it is not invasive, not uropathogenic (Sonnenborn et al., 2009). As early as in 1917, E. coli Nissle was packaged into medicinal capsules, called Mutaflor, for therapeutic use. It is commonly accepted that E. coli Nissle's therapeutic efficacy and safety have convincingly been proven (Ukena et al., 2007).

In one embodiment, the engineered bacterial cell does not colonize the subject.

One of ordinary skill in the art would appreciate that the genetic modifications disclosed herein may be adapted for other species, strains, and subtypes of bacteria. Furthermore, genes from one or more different species can be introduced into one another, e.g., a gene from Lactobacillus plantarum or Methanobrevibacter smithii 3142 can be expressed in Escherichia coli.

In some embodiments, the bacterial cell is a genetically engineered bacterial cell. In another embodiment, the bacterial cell is an engineered bacterial cell. In some embodiments, the disclosure comprises a colony of bacterial cells.

In another aspect, the disclosure provides an engineered bacterial culture which comprises engineered bacterial cells.

In some embodiments of the above described genetically engineered bacteria, the gene or gene cassette(s) are present on a plasmid in the bacterium and operatively linked on the plasmid to the promoter that is induced under low-oxygen or anaerobic conditions. In other embodiments, the gene or gene cassette(s) is present in the bacterial chromosome and is operatively linked in the chromosome to the promoter that is induced under low-oxygen or anaerobic conditions.

In some embodiments, the genetically engineered bacterium is an auxotroph or a conditional auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyA and ΔdapA auxotroph.

In some embodiments, the genetically engineered bacteria further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of promoter having a TetR repressor binding site and a gene encoding the TetR under the control of an inducible promoter that is induced by arabinose, such as P_{araBAD}. In some embodiments, the genetically engineered bacteria further comprise one or more genes encoding an antitoxin.

In some embodiments, the genetically engineered bacterium is an auxotroph and further comprises a kill-switch circuit, such as any of the kill-switch circuits described herein.

In some embodiments of the above described genetically engineered bacteria, the gene or gene cassette(s) are present on a plasmid in the bacterium and operatively linked on the plasmid to the promoter that is induced under low-oxygen or anaerobic conditions. In other embodiments, the gene or gene cassette(s) are present in the bacterial chromosome and is operatively linked in the chromosome to the promoter that is induced under low-oxygen or anaerobic conditions.

In one aspect, the disclosure provides an engineered bacterial culture which reduces levels of propionate, propionyl CoA, methylmalonate and/or methylmalonyl CoA in the media of the culture. In one embodiment, the levels of the propionate, propionyl CoA, methylmalonate and/or methylmalonyl CoA are reduced by about 50%, about 75%, or about 100% in the media of the cell culture. In another embodiment, the levels of the propionate, propionyl CoA, methylmalonate and/or methylmalonyl CoA are reduced by about two-fold, three-fold, four-fold, five-fold, six-fold, seven-fold, eight-fold, nine-fold, or ten-fold in the media of the cell culture. In one embodiment, the levels of the propionate, propionyl CoA, methylmalonate and/or methylmalonyl CoA are reduced below the limit of detection in the media of the cell culture. In some embodiments, such metabolites, e.g., propionate, propionyl CoA, methylmalonate, and/or methylmalonyl CoA are added to the medium and reduction of these metabolites is measured, e.g., to determine in vitro activity of the engineered bacterial cultures.

The genetically engineered microorganisms, or programmed microorganisms, such as genetically engineered bacteria of the disclosure are capable of producing one or more enzymes for metabolizing propionate and/or metabolizing one or more propionate metabolite(s). Non-limiting examples of such enzymes and propionate metabolic pathways are described herein. For example, propionate metabolic pathways include, but are not limited to, one or more of the polyhydroxyalkanoate (PHA), methylmalonyl-CoA (MMCA), and 2-methylcitrate (2MC) pathways, e.g., as described herein. In some aspects, the disclosure provides a bacterial cell that comprises one or more heterologous gene sequence(s) and/or gene cassette(s) encoding one or more propionate catabolism enzyme(s) or other protein(s) that results in a decrease in levels of propionate and/or certain propionate metabolites, e.g., methylmalonate.

In certain embodiments, the genetically engineered bacteria are obligate anaerobic bacteria. In certain embodiments, the genetically engineered bacteria are facultative anaerobic bacteria. In certain embodiments, the genetically engineered bacteria are aerobic bacteria. In some embodiments, the genetically engineered bacteria are Gram-positive bacteria. In some embodiments, the genetically engineered bacteria are Gram-positive bacteria and lack LPS. In some embodiments, the genetically engineered bacteria are Gram-negative bacteria. In some embodiments, the genetically engineered bacteria are Gram-positive and obligate anaerobic bacteria. In some embodiments, the genetically engineered bacteria are Gram-positive and facultative anaerobic bacteria. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the genetically engineered bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus lactis bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell.

In some embodiments, the genetically engineered bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be adapted for other species, strains, and subtypes of bacteria. Furthermore, genes from one or more different species can be introduced into one another, e.g., the phaBCA genes from Acinetobacter sp RA3849, the accA gene from Streptopmyces coelicolor, pccB gene from Streptopmyces coelicolor, mmcE gene from Propionibcterium freudenreichii or the mutAB genes from Propionibcterium freudenreichii, or matB, derived from Rhodopseudomonas palustris, can be expressed in Escherichia coli. In some embodiments, the genes are codon optimized, e.g., for expression in E. coli. In one embodiment, the recombinant bacterial cell does not colonize the subject having the disorder. Unmodified E. coli Nissle and the genetically engineered bacteria of the invention may be destroyed, e.g., by defense factors in the gut or blood serum (Sonnenborn et al., 2009). In some embodiments, the residence time is calculated for a human subject. In some embodiments, residence time in vivo is calculated for the genetically engineered bacteria of the invention.

In some embodiments, the bacterial cell is a genetically engineered bacterial cell. In another embodiment, the bacterial cell is a recombinant bacterial cell. In some embodiments, the disclosure comprises a colony of bacterial cells disclosed herein.

In another aspect, the disclosure provides a recombinant bacterial culture which comprises bacterial cells disclosed herein. In one aspect, the disclosure provides a recombinant bacterial culture which reduces levels of propionate in the media of the culture. In one embodiment, the levels of propionate and/or one or more of its metabolites are reduced by about 50%, about 75%, or about 100% in the media of the cell culture. In another embodiment, the levels of propionate and/or one or more of its metabolites, are reduced by about two-fold, three-fold, four-fold, five-fold, six-fold, seven-fold, eight-fold, nine-fold, or ten-fold in the media of the cell culture. In one embodiment, the levels of propionate and/or one or more of its metabolites are reduced below the limit of detection in the media of the cell culture.

In some embodiments of the above described genetically engineered bacteria, the gene encoding a propionate catabolism enzyme is present on a plasmid in the bacterium and operatively linked on the plasmid to a promoter that is induced under low-oxygen or anaerobic conditions, such as any of the promoters disclosed herein. In other embodiments, the gene encoding a propionate catabolism enzyme is present in the bacterial chromosome and is operatively linked in the chromosome to the promoter that is induced under low-oxygen or anaerobic conditions, such as any of the promoters disclosed herein. In some embodiments of the above described genetically engineered bacteria, the gene encoding a propionate catabolism enzyme is present on a plasmid in the bacterium and operatively linked on the plasmid to the promoter that is induced under inflammatory conditions, such as any of the promoters disclosed herein. In other embodiments, the gene encoding a propionate catabolism enzyme is present in the bacterial chromosome and is operatively linked in the chromosome to the promoter that is induced under inflammatory conditions, such as any of the promoters disclosed herein.

In some embodiments, the genetically engineered bacteria comprising gene sequence encoding a propionate catabolism enzyme is an auxotroph. In one embodiment, the genetically engineered bacterium is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil auxotroph. In some embodiments, the engineered bacteria have more than one auxotrophy, for example, they may be a ΔthyA and ΔdapA auxotroph. In some embodiments, the genetically engineered bacteria comprising gene sequence encoding a propionate catabolism enzyme lacks functional ilvC gene sequence, e.g., is a ilvC auxotroph. IlvC encodes keto acid reductoisomerase, which enzyme is required for propionate synthesis. Knock out of ilvC creates an auxotroph and requires the bacterial cell to import isoleucine and valine to survive.

In some embodiments, the genetically engineered bacteria comprising gene sequence encoding a propionate catabolism enzyme further comprise gene sequence(s) encoding a propionate transporter into the bacterial cell. In certain embodiments, the propionate transporter is MctC, PutP_6, or any other propionate transporters described herein. In certain embodiments, the bacterial cell contains gene sequence encoding MctC, PutP_6, or any other propionate transporters described herein.

In some embodiments, the genetically engineered bacteria comprising gene sequence encoding a propionate catabolism enzyme further comprise gene sequence(s) encoding a secretion protein or protein complex for secreting a biomolecule, such as any of the secretion systems disclosed herein.

In some embodiments, the genetically engineered bacteria comprising gene sequence encoding a propionate catabolism enzyme further comprise gene sequence(s) encoding one or more antibiotic gene(s), such as any of the antibiotic genes disclosed herein.

In some embodiments, the genetically engineered bacteria comprising a propionate catabolism enzyme further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of a promoter having a TetR repressor binding site and a gene encoding the TetR under the control of an inducible promoter that is induced by arabinose, such as ParaBAD. In some embodiments, the genetically engineered bacteria further comprise one or more genes encoding an antitoxin.

In some embodiments, the genetically engineered bacterium is an auxotroph comprising gene sequence encoding a propionate catabolism enzyme and further comprises a kill-switch circuit, such as any of the kill-switch circuits described herein.

In some embodiments of the above described genetically engineered bacteria, the gene encoding a propionate catabolism enzyme is present on a plasmid in the bacterium. In some embodiments, the gene encoding a propionate catabolism enzyme is present in the bacterial chromosome. In some embodiments, the gene sequence(s) encoding a propionate transporter, e.g., MctC, PutP_6, or any other propionate transporters described herein, is present on a plasmid in the bacterium. In some embodiments, the gene sequence(s) encoding a propionate transporter, e.g., MctC, PutP_6, or any other propionate transporters described herein, is present in the bacterial chromosome. In some embodiments, the gene sequence encoding a secretion protein or protein complex for secreting a biomolecule, such as any of the secretion systems disclosed herein, is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

Inducible Promoters

In some embodiments, the bacterial cell comprises a stably maintained plasmid or chromosome carrying the gene encoding the propionate catabolism enzyme such that the propionate catabolism enzyme can be expressed in the host cell, and the host cell is capable of survival and/or growth in vitro, e.g., in medium, and/or in vivo, e.g., in the gut. In some embodiments, bacterial cell comprises two or more distinct propionate catabolism enzymes. In some embodiments, the genetically engineered bacteria comprise multiple copies of the same propionate catabolism enzyme gene. In some embodiments, the genetically engineered bacteria comprise multiple copies of different propionate catabolism enzyme genes or gene cassette(s). In some embodiments, the gene(s) encoding the propionate catabolism enzyme is present on a plasmid and operably linked to a directly or indirectly inducible promoter. In some embodiments, the gene encoding the propionate catabolism enzyme is present on a plasmid and operably linked to a promoter that is induced under low-oxygen or anaerobic conditions. In some embodiments, the gene encoding the propionate catabolism enzyme is present on a chromosome and operably linked to a directly or indirectly inducible promoter. In some embodiments, the gene encoding the propionate catabolism enzyme is present in the chromosome and operably linked to a promoter that is induced under low-oxygen or anaerobic conditions. In some embodiments, the gene encoding the propionate catabolism enzyme is present on a plasmid and operably linked to a promoter that is induced by exposure to tetracycline or arabinose.

In some embodiments, the bacterial cell comprises a stably maintained plasmid or chromosome carrying the at least one gene encoding a transporter of propionate and/or one or more metabolites thereof, such that the transporter, can be expressed in the host cell, and the host cell is capable of survival and/or growth in vitro, e.g., in medium, and/or in vivo, e.g., in the gut. In some embodiments, bacterial cell comprises two or more distinct copies of the at least one gene encoding a propionate transporter. In some embodiments, the genetically engineered bacteria comprise multiple copies of the same at least one gene encoding a propionate transporter. In some embodiments, the at least one gene encoding a transporter of propionate, is present on a plasmid and operably linked to a directly or indirectly inducible promoter. In some embodiments, the at least one gene encoding a propionate transporter, is present on a plasmid and operably linked to a promoter that is induced under low-oxygen or anaerobic conditions. In some embodiments, the at least one gene encoding a propionate transporter, is present on a chromosome and operably linked to a directly or indirectly inducible promoter. In some embodiments, the at least one gene encoding a propionate transporter, is present in the chromosome and operably linked to a promoter that is induced under low-oxygen or anaerobic conditions. In some embodiments, the at least one gene encoding a transporter propionate and/or methylmalonate, is present on a plasmid and operably linked to a promoter that is induced by exposure to tetracycline or arabinose.

In some embodiments, the promoter that is operably linked to the gene encoding the propionate catabolism enzyme and the promoter that is operably linked to the gene encoding the propionate transporter, is directly induced by exogenous environmental conditions. In some embodiments, the promoter that is operably linked to the gene encoding the propionate catabolism enzyme and the promoter that is operably linked to the gene encoding the propionate transporter, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e g, propionate. In some embodiments, the promoter is directly or indirectly induced by a molecule that is co-administered with the bacterial cell.

In some embodiments, the bacterial cell comprises a stably maintained plasmid or chromosome carrying the at least one gene encoding a propionate binding protein, such that the propionate binding protein, can be expressed in the host cell, and the host cell is capable of survival and/or growth in vitro, e.g., in medium, and/or in vivo, e.g., in the gut. In some embodiments, bacterial cell comprises two or more distinct copies of the at least one gene encoding a propionate binding protein. In some embodiments, the genetically engineered bacteria comprise multiple copies of the same at least one gene encoding a propionate binding protein. In some embodiments, the at least one gene encoding a propionate binding protein is present on a plasmid and operably linked to a directly or indirectly inducible promoter. In some embodiments, the at least one gene encoding a propionate binding protein, is present on a plasmid and operably linked to a promoter that is induced under low-oxygen or anaerobic conditions. In some embodiments, the at least one gene encoding a propionate binding protein, is present on a chromosome and operably linked to a directly or indirectly inducible promoter. In some embodiments, the at least one gene encoding a propionate binding protein, is present in the chromosome and operably linked to a promoter that is induced under low-oxygen or anaerobic conditions. In some embodiments, the at least one gene encoding a propionate binding protein, is present on a plasmid and operably linked to a promoter that is induced by exposure to tetracycline or arabinose.

In some embodiments, the promoter that is operably linked to the gene encoding the propionate catabolism enzyme and the promoter that is operably linked to the gene encoding the propionate binding protein, is directly induced by exogenous environmental conditions. In some embodiments, the promoter that is operably linked to the gene encoding the propionate catabolism enzyme and the promoter that is operably linked to the gene encoding the propionate binding protein, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e.g., propionate. In some embodiments, the promoter is directly or indirectly induced by a molecule that is co-administered with the bacterial cell.

### FNR Dependent Regulation

In certain embodiments, the bacterial cell comprises a gene encoding a propionate catabolism enzyme is expressed under the control of the fumarate and nitrate reductase regulator (FNR) promoter. In certain embodiments, the bacterial cell comprises at least one gene encoding a propionate transporter is expressed under the control of the fumarate and nitrate reductase regulator (FNR) promoter. In certain embodiments, the bacterial cell comprises at least one gene encoding a propionate binding protein is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR responsive promoters include, but are not limited to, the FNR responsive promoters listed in the chart, below. Underlined sequences are predicted ribosome binding sites, and bolded sequences are restriction sites used for cloning.

TABLE 3
FNR responsive promoters

| FNR Responsive | |
|---|---|
| Promoter | Sequence |
| SEQ ID NO: 1 | GTCAGCATAACACCCTGACCTCTCATTAATTGTTCATGCCGGGCGGCACTATCGTCGTCCGGCCT |
| | TTTCCTCTCTTACTCTGCTACGTACATCTATTTCTATAAATCCGTTCAATTTGTCTGTTTTTTGCACA |
| | AACATGAAATATCAGACAATTCCGTGACTTAAGAAAATTTATACAAATCAGCAATATACCCCTTA |
| | AGGAGTATATAAAGGTGAATTTGATTTACATCAATAAGCGGGGTTGCTGAATCGTTAAGGTAGG |
| | CGGTAATAGAAAAGAAATCGAGGCAAAA |
| SEQ ID NO: 2 | ATTTCCTCTCATCCCATCCGGGGTGAGAGTCTTTTCCCCCGACTTATGGCTCATGCATGCATCAAA |
| | AAAGATGTGAGCTTGATCAAAAACAAAAAATATTTCACTCGACAGGAGTATTTATATTGCGCCCG |
| | TTACGTGGGCTTCGACTGTAAATCAGAAAGGAGAAAACACCT |
| SEQ ID NO: 3 | GTCAGCATAACACCCTGACCTCTCATTAATTGTTCATGCCGGGCGGCACTATCGTCGTCCGGCCT |
| | TTTCCTCTCTTACTCTGCTACGTACATCTATTTCTATAAATCCGTTCAATTTGTCTGTTTTTTGCACA |
| | AACATGAAATATCAGACAATTCCGTGACTTAAGAAAATTTATACAAATCAGCAATATACCCCTTA |
| | AGGAGTATATAAAGGTGAATTTGATTTACATCAATAAGCGGGGTTGCTGAATCGTTAAGGATCC |
| | CTCTAGAAATAATTTTGTTTAACTTTAAGAAGGAGATATACAT |
| SEQ ID NO: 4 | CATTTCCTCTCATCCCATCCGGGGTGAGAGTCTTTTCCCCCGACTTATGGCTCATGCATGCATCAA |
| | AAAAGATGTGAGCTTGATCAAAAACAAAAAATATTTCACTCGACAGGAGTATTTATATTGCGCCC |
| | GGATCCCTCTAGAAATAATTTTGTTTAACTTTAAGAAGGAGATATACAT |
| SEQ ID NO: 5 | AGTTGTTCTTATTGGTGGTGTTGCTTTATGGTTGCATCGTAGTAAATGGTTGTAACAAAAGCAAT |
| | TTTTCCGGCTGTCTGTATACAAAAACGCCGTAAAGTTTGAGCGAAGTCAATAAACTCTCTACCCA |
| | TTCAGGGCAATATCTCTCTTGGATCCCTCTAGAAATAATTTTGTTTAACTTTAAGAAGGAGATATA |
| | CAT |
| SEQ ID NO: 6 | ATCCCCATCACTCTTGATGGAGATCAATTCCCCAAGCTGCTAGAGCGTTACCTTGCCCTTAAACAT |
| | TAGCAATGTCGATTTATCAGAGGGCCGACAGGCTCCCACAGGAGAAAACCG |
| SEQ ID NO: 7 | CTCTTGATCGTTATCAATTCCCACGCTGTTTCAGAGCGTTACCTTGCCCTTAAACATTAGCAATGT |
| | CGATTTATCAGAGGGCCGACAGGCTCCCACAGGAGAAAACCG |

TABLE 4
FNR Promoter Sequences

| SEQ ID NO | FNR-responsive regulatory region Sequence |
|---|---|
| nirB1 | GTCAGCATAACACCCTGACCTCTCATTAATTGTTCATGCCGGGCGGCACT |
| SEQ ID NO: 8 | ATCGTCGTCCGGCCTTTTCCTCTCTTACTCTGCTACGTACATCTATTTCT |
| | ATAAATCCGTTCAATTTGTCTGTTTTTTGCACAAACATGAAATATCAGAC |
| | AATTCCGTGACTTAAGAAAATTTATACAAATCAGCAATATACCCCTTAAG |
| | GAGTATATAAAGGTGAATTTGATTTACATCAATAAGCGGGGTTGCTGAAT |
| | CGTTAAGGTAGGCGGTAATAGAAAAGAAATCGAGGCAAAA |
| nirB2 | CGGCCCGATCGTTGAACATAGCGGTCCGCAGGCGGCACTGCTTACAGCAA |
| SEQ ID NO: 9 | ACGGTCTGTACGCTGTCGTCTTTGTGATGTGCTTCCTGTTAGGTTTCGTC |
| | AGCCGTCACCGTCAGCATAACACCCTGACCTCTCATTAATTGCTCATGCC |
| | GGACGGCACTATCGTCGTCCGGCCTTTTCCTCTCTTCCCCCGCTACGTGC |
| | ATCTATTTCTATAAACCCGCTCATTTTGTCTATTTTTTGCACAAACATGA |
| | AATATCAGACAATTCCGTGACTTAAGAAAATTTATACAAATCAGCAATAT |
| | ACCCATTAAGGAGTATATAAAGGTGAATTTGATTTACATCAATAAGCGGG |
| | GTTGCTGAATCGTTAAGGTAGGCGGTAATAGAAAAGAAATCGAGGCAAAA |
| | atgtttgtttaactttaagaaggagatatacat |
| nirB3 | GTCAGCATAACACCCTGACCTCTCATTAATTGCTCATGCCGGACGGCACT |
| SEQ ID NO: 10 | ATCGTCGTCCGGCCTTTTCCTCTCTTCCCCCGCTACGTGCATCTATTTCT |
| | ATAAACCCGCTCATTTTGTCTATTTTTTGCACAAACATGAAATATCAGAC |
| | AATTCCGTGACTTAAGAAAATTTATACAAATCAGCAATATACCCATTAAG |
| | GAGTATATAAAGGTGAATTTGATTTACATCAATAAGCGGGGTTGCTGAAT |
| | CGTTAAGGTAGGCGGTAATAGAAAAGAAATCGAGGCAAAA |
| ydfZ | ATTTCCTCTCATCCCATCCGGGGTGAGAGTCTTTTCCCCCGACTTATGGC |
| SEQ ID NO: 11 | TCATGCATGCATCAAAAAAGATGTGAGCTTGATCAAAAACAAAAAATATT |
| | TCACTCGACAGGAGTATTTATATTGCGCCCGTTACGTGGGCTTCGACTGT |
| | AAATCAGAAAGGAGAAAACACCT |
| nirB + RBS | GTCAGCATAACACCCTGACCTCTCATTAATTGTTCATGCCGGGCGGCACT |
| SEQ ID NO: 12 | ATCGTCGTCCGGCCTTTTCCTCTCTTACTCTGCTACGTACATCTATTTCT |
| | ATAAATCCGTTCAATTTGTCTGTTTTTTGCACAAACATGAAATATCAGAC |
| | AATTCCGTGACTTAAGAAAATTTATACAAATCAGCAATATACCCCTTAAG |
| | GAGTATATAAAGGTGAATTTGATTTACATCAATAAGCGGGGTTGCTGAAT |
| | CGTTAAGGATCCCTCTAGAAATAATTTTGTTTAACTTTAAGAAGGAGATA |
| | TACAT |
| ydfZ + RBS | CATTTCCTCTCATCCCATCCGGGGTGAGAGTCTTTTCCCCCGACTTATGG |
| SEQ ID NO: 13 | CTCATGCATGCATCAAAAAAGATGTGAGCTTGATCAAAAACAAAAAATAT |
| | TTCACTCGACAGGAGTATTTATATTGCGCCCGGATCCCTCTAGAAATAAT |
| | TTTGTTTAACTTTAAGAAGGAGATATACAT |
| fnrS1 | AGTTGTTCTTATTGGTGGTGTTGCTTTATGGTTGCATCGTAGTAAATGGT |
| SEQ ID NO: 14 | TGTAACAAAAGCAATTTTTCCGGCTGTCTGTATACAAAAACGCCGTAAAG |
| | TTTGAGCGAAGTCAATAAACTCTCTACCCATTCAGGGCAATATCTCTCTT |
| | GGATCCCTCTAGAAATAATTTTGTTTAACTTTAAGAAGGAGATATACAT |
| fnrS2 | AGTTGTTCTTATTGGTGGTGTTGCTTTATGGTTGCATCGTAGTAAATGGT |
| SEQ ID NO: 15 | TGTAACAAAAGCAATTTTTCCGGCTGTCTGTATACAAAAACGCCGCAAAG |
| | TTTGAGCGAAGTCAATAAACTCTCTACCCATTCAGGGCAATATCTCTCTT |
| | GGATCCAAAGTGAACTCTAGAAATAATTTTGTTTAACTTTAAGAAGGAGA |
| | TATACAT |
| nirB + crp | TCGTCTTTGTGATGTGCTTCCTGTTAGGTTTCGTCAGCCGTCACCGTCAG |
| SEQ ID NO: 16 | CATAACACCCTGACCTCTCATTAATTGCTCATGCCGGACGGCACTATCGT |
| | CGTCCGGCCTTTTCCTCTCTTCCCCCGCTACGTGCATCTATTTCTATAAA |
| | CCCGCTCATTTTGTCTATTTTTTGCACAAACATGAAATATCAGACAATTC |
| | CGTGACTTAAGAAAATTTATACAAATCAGCAATATACCCATTAAGGAGTA |
| | TATAAAGGTGAATTTGATTTACATCAATAAGCGGGGTTGCTGAATCGTTA |
| | AGGTAGaaatgtgatctagttcacatttGCGGTAATAGAAAAGAAATCGA |
| | GGCAAAAatgtttgtttaactttaagaaggagatatacat |
| fnrS + crp | AGTTGTTCTTATTGGTGGTGTTGCTTTATGGTTGCATCGTAGTAAATGGT |
| SEQ ID NO: 17 | TGTAACAAAAGCAATTTTTCCGGCTGTCTGTATACAAAAACGCCGCAAAG |
| | TTTGAGCGAAGTCAATAAACTCTCTACCCATTCAGGGCAATATCTCTCaa |
| | atgtgatctagttcacattttttgtttaactttaagaaggagatatacat |

In one embodiment, the FNR responsive promoter comprises SEQ ID NO: 1. In another embodiment, the FNR responsive promoter comprises SEQ ID NO: 2. In another embodiment, the FNR responsive promoter comprises SEQ ID NO: 3. In another embodiment, the FNR responsive promoter comprises SEQ ID NO: 4. In yet another embodiment, the FNR responsive promoter comprises SEQ ID NO: 5. In yet another embodiment, the FNR responsive promoter comprises SEQ ID NO: 6. In yet another embodiment, the FNR responsive promoter comprises SEQ ID NO: 7. In yet another embodiment, the FNR responsive promoter comprises SEQ ID NO: 8. In yet another embodiment, the FNR responsive promoter comprises SEQ ID NO: 9. In yet another embodiment, the FNR responsive promoter comprises SEQ ID NO: 10. In yet another embodiment, the FNR responsive promoter comprises SEQ ID NO: 11. In yet another embodiment, the FNR responsive promoter comprises SEQ ID NO: 12. In yet another embodiment, the FNR responsive promoter comprises SEQ ID NO: 13. In yet another embodiment, the FNR responsive promoter comprises SEQ ID NO: 14. In yet another embodiment, the FNR responsive promoter comprises SEQ ID NO: 15. In yet another embodiment, the FNR responsive promoter comprises SEQ ID NO: 16. In yet another embodiment, the FNR responsive promoter comprises SEQ ID NO: 17.

In other embodiments, the FNR responsive promoter has at least about 80% identity with a nucleic acid sequence encoding any of SEQ ID NOs:1-17. In other embodiments, the FNR responsive promoter has at least about 85% identity with a nucleic acid sequence encoding any of SEQ ID NOs:1-17. In other embodiments, the FNR responsive promoter has at least about 90% identity with a nucleic acid sequence encoding any of SEQ ID NOs:1-17. In other embodiments, the FNR responsive promoter has at least about 95% identity with a nucleic acid sequence encoding any of SEQ ID NOs:1-17. In other embodiments, the FNR responsive promoter has at least about 96%, 97%, 98%, or 99% identity with a nucleic acid sequence encoding any of SEQ ID NOs:1-17. Accordingly, in some embodiments, the FNR responsive promoter has at least about 80%, 81%, 82%, 83%, 84%, 85%, 86%, 87%, 88%, 89%, 90%, 91%, 92%, 93%, 94%, 95%, 96%, 97%, 98%, or 99% identity with a nucleic acid sequence encoding any of SEQ ID NOs:1-43.

In some embodiments, multiple distinct FNR nucleic acid sequences are inserted in the genetically engineered bacteria. In alternate embodiments, the genetically engineered bacteria comprise a gene encoding a propionate catabolism enzyme disclosed herein which is expressed under the control of an alternate oxygen level-dependent promoter, e.g., DNR (Trunk et al., 2010) or ANR (Ray et al., 1997). In alternate embodiments, the genetically engineered bacteria comprise at least one gene encoding a propionate transporter which is expressed under the control of an alternate oxygen level-dependent promoter, e.g., DNR (Trunk et al., 2010) or ANR (Ray et al., 1997). In alternate embodiments, the genetically engineered bacteria comprise at least one gene encoding a propionate binding protein which is expressed under the control of an alternate oxygen level-dependent promoter, e.g., DNR (Trunk et al., 2010) or ANR (Ray et al., 1997). In these embodiments, catabolism of propionate and/or its metabolites is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the propionate catabolism enzyme, and/or the at least one gene encoding a propionate transporter, and/or the at least one gene encoding a propionate binding protein in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the propionate catabolism enzyme, and/or the at least one gene encoding a propionate transporter and/or the at least one gene encoding a propionate binding protein in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the propionate catabolism enzyme, and/or the at least one gene encoding a propionate transporter, and/or the at least one gene encoding a propionate binding protein in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., 2006).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the propionate catabolism enzyme are present on different plasmids. In some embodiments, the gene encoding the oxygen level-sensing transcriptional regulator and the gene encoding the propionate catabolism enzyme and/or the at least one gene encoding a propionate transporter and/or the at least one gene encoding a propionate binding protein are present on different plasmids. In some embodiments, the gene encoding the oxygen level-sensing transcriptional regulator and the gene encoding the propionate catabolism enzyme and/or the at least one gene encoding a transporter of a propionate and/or the at least one gene encoding a propionate binding protein are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the gene encoding the propionate catabolism enzyme and/or the at least one gene encoding a propionate transporter and/or the at least one gene encoding a propionate binding protein are present on different chromosomes. In some embodiments, the gene encoding the oxygen level-sensing transcriptional regulator and the gene encoding the propionate catabolism enzyme and/or the at least one gene encoding a propionate transporter and/or the at least one gene encoding a propionate binding protein are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the propionate catabolism enzyme and/or the transporter of propionate and/or metabolites thereof and/or the propionate binding protein. In some embodiments, expression of the transcriptional regulator is controlled by the same promoter that controls expression of the propionate catabolism enzyme and/or the transporter of propionate and/or metabolites thereof, and/or the propionate binding protein. In some embodiments, the transcriptional regulator and the propionate catabolism enzyme are divergently transcribed from a promoter region.

### RNS Dependent Regulation

In some embodiments, the genetically engineered bacteria comprise a gene encoding a propionate catabolism enzyme that is expressed under the control of an inducible promoter. In some embodiments, the genetically engineered bacterium that expresses a propionate catabolism enzyme and/or a transporter of propionate and/or metabolites thereof and/or propionate binding protein is under the control of a promoter that is activated by inflammatory conditions. In one embodiment, the gene for producing the propionate catabolism enzyme and/or a transporter of propionate and/or metabolites thereof and/or propionate binding protein is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO•), peroxynitrite or peroxynitrite anion (ONOO—), nitrogen dioxide (•NO2), dinitrogen trioxide (N2O3), peroxynitrous acid (ONOOH), and nitroperoxycarbonate (ONOOCO2-) (unpaired electrons denoted by •). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., a propionate catabolism enzyme gene sequence(s), e.g., any of the propionate catabolism enzymes described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or genes, e.g., propionate catabolism enzyme gene sequence(s), propionate transporter sequence(s), propionate binding protein(s). For example, in the presence of RNS, a transcription factor senses RNS and no longer binds to and/or represses the regulatory region, thereby derepressing an operatively linked gene sequence or gene cassette. Thus, RNS derepresses expression of the gene or genes.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 5.

TABLE 5
Examples of RNS-sensing transcription factors and
RNS-responsive genes

| RNS-sensing | Primarily | |
|---|---|---|
| transcription | capable of | Examples of responsive genes, |
| factor: | sensing: | promoters, and/or regulatory regions: |
| NsrR | NO | norB, aniA, nsrR, hmpA, ytfE, ygbA, hcp, |
| | | hcr, nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of a propionate catabolism enzyme, propionate transporter, and/or propionate binding protein, thus controlling expression of the propionate catabolism enzyme, propionate transporter, and/or propionate binding protein relative to RNS levels. For example, the tunable regulatory region is a RNS-inducible regulatory region, and the payload is an propionate catabolism enzyme, propionate transporter, and/or propionate binding protein, such as any of the propionate catabolism enzymes, propionate transporters, and propionate binding proteins provided herein; when RNS is present, e.g., in an inflamed tissue, a RNS-sensing transcription factor binds to and/or activates the regulatory region and drives expression of the propionate catabolism enzyme, propionate transporter, and/or propionate binding protein gene or genes. Subsequently, when inflammation is ameliorated, RNS levels are reduced, and production of the propionate catabolism enzyme, propionate transporter, and/or propionate binding protein is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The genetically engineered bacteria of the invention may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012; Table 5). In certain embodiments, the genetically engineered bacteria of the invention comprise a RNS-inducible regulatory region from norVW that is operatively linked to a gene or genes, e.g., one or more propionate catabolism enzyme, propionate transporter, and/or propionate binding protein gene sequence(s). In the presence of RNS, a NorR transcription factor senses RNS and activates to the norVW regulatory region, thereby driving expression of the operatively linked gene(s) and producing the propionate catabolism enzyme, propionate transporter, and/or propionate binding protein.

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The genetically engineered bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008; Table 5). In certain embodiments, the genetically engineered bacteria of the invention comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more propionate catabolism enzymes. In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The genetically engineered bacteria of the invention may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010; Table 5). In certain embodiments, the genetically engineered bacteria of the invention comprise a RNS-derepressible regulatory region from norB that is operatively linked to a gene or genes, e.g., a propionate catabolism enzyme gene or genes. In the presence of RNS, an NsrR transcription factor senses RNS and no longer binds to the norB regulatory region, thereby derepressing the operatively linked propionate catabolism enzyme, propionate transporter, and/or propionate binding protein gene or genes and producing the encoding an propionate catabolism enzyme(s).

In some embodiments, it is advantageous for the genetically engineered bacteria to express a RNS-sensing transcription factor that does not regulate the expression of a significant number of native genes in the bacteria. In some embodiments, the genetically engineered bacterium of the invention expresses a RNS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the genetically engineered bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the genetically engineered bacteria may comprise a two repressor activation regulatory circuit, which is used to express a propionate catabolism enzyme. The two repressor activation regulatory circuit comprises a first RNS-sensing repressor and a second repressor, which is operatively linked to a gene or gene cassette, e.g., encoding a propionate catabolism enzyme. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., a propionate catabolism enzyme, propionate transporter, and/or propionate binding protein gene or genes is expressed.

A RNS-responsive transcription factor may induce, derepress, or repress gene expression depending upon the regulatory region sequence used in the genetically engineered bacteria. One or more types of RNS-sensing transcription factors and corresponding regulatory region sequences may be present in genetically engineered bacteria. In some embodiments, the genetically engineered bacteria comprise one type of RNS-sensing transcription factor, e.g., NsrR, and one corresponding regulatory region sequence, e.g., from norB. In some embodiments, the genetically engineered bacteria comprise one type of RNS-sensing transcription factor, e.g., NsrR, and two or more different corresponding regulatory region sequences, e.g., from norB and aniA. In some embodiments, the genetically engineered bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the genetically engineered bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the genetically engineered bacteria of the invention comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the genetically engineered bacteria of the invention comprise a gene for a RNS-sensing transcription factor from a different species, strain, or substrain of bacteria. In some embodiments, the genetically engineered bacteria comprise a RNS-responsive regulatory region from a different species, strain, or substrain of bacteria. In some embodiments, the genetically engineered bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the genetically engineered bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the genetically engineered bacteria of the invention comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the genetically engineered bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the propionate catabolism enzyme in the presence of RNS, as compared to the wild-type regulatory region under the same conditions. In some embodiments, the genetically engineered bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the propionate catabolism enzyme in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the propionate catabolism enzyme, propionate transporter, and/or propionate binding protein in the presence of RNS.

In some embodiments, the gene or gene cassette for producing the anti-inflammation and/or gut barrier function enhancer molecule is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of a propionate catabolism enzyme, propionate transporter, and/or propionate binding protein gene(s) may be integrated into the bacterial chromosome. Having multiple copies of the gene or gen(s) integrated into the chromosome allows for greater production of the propionate catabolism enzyme(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

### ROS-Dependent Regulation

In some embodiments, the genetically engineered bacteria comprise a gene for producing a propionate catabolism enzyme, propionate transporter, and/or propionate binding protein that is expressed under the control of an inducible promoter. In some embodiments, the genetically engineered bacterium that expresses a propionate catabolism enzyme, propionate transporter, and/or propionate binding protein under the control of a promoter that is activated by conditions of cellular damage. In one embodiment, the gene for producing the propionate catabolism enzyme is expressed under the control of a cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H2O2), organic peroxide (ROOH), hydroxyl ion (OH—), hydroxyl radical (•OH), superoxide or superoxide anion (•O2-), singlet oxygen (1O2), ozone (O3), carbonate radical, peroxide or peroxyl radical (•O2-2), hypochlorous acid (HOCl), hypochlorite ion (OCl—), sodium hypochlorite (NaOCl), nitric oxide (NO•), and peroxynitrite or peroxynitrite anion (ONOO—) (unpaired electrons denoted by •). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more propionate catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or genes, e.g., one or more genes encoding one or more propionate catabolism enzyme(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 6.

TABLE 6
Examples of ROS-sensing transcription factors and
ROS-responsive genes

| ROS-sensing | Primarily | |
|---|---|---|
| transcription | capable of | Examples of responsive genes, |
| factor: | sensing: | promoters, and/or regulatory regions: |
| OxyR | H₂O₂ | ahpC; ahpF; dps; dsbG; fhuF; flu; fur; |
| | | gor; grxA; hemH; katG; oxyS; sufA; |
| | | sufB; sufC; sufD; sufE; sufS; trxC; uxuA; |
| | | yaaA; yaeH; yaiA; ybjM; ydcH; ydeN; |
| | | ygaQ; yljA; ytfK |
| PerR | H₂O₂ | katA; ahpCF; mrgA; zoaA; fur; |
| | | hemAXCDBL; srfA |
| OhrR | Organic | ohrA |
| | peroxides | |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; |
| | | tnp2d; phoD; tnp15b; pstA; tnp5b; xylC; |
| | | gabD1; rluC2; cgtS9; azlC; narKGHJI; |
| | | rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of a propionate catabolism enzyme, thus controlling expression of the propionate catabolism enzyme relative to ROS levels. For example, the tunable regulatory region is a ROS-inducible regulatory region, and the molecule is a propionate catabolism enzyme; when ROS is present, e.g., in an inflamed tissue, a ROS-sensing transcription factor binds to and/or activates the regulatory region and drives expression of the gene sequence for the propionate catabolism enzyme, propionate transporter, and/or propionate binding protein thereby producing the propionate catabolism enzyme, propionate transporter, and/or propionate binding protein. Subsequently, when inflammation is ameliorated, ROS levels are reduced, and production of the propionate catabolism enzyme, propionate transporter, and/or propionate binding protein is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H2O2 detoxification (katE, ahpCF), heme biosynthesis (hemH), reductant supply (grxA, gor, trxC), thiol-disulfide isomerization (dsbG), Fe—S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012; Table 6). In certain embodiments, the genetically engineered bacteria of the invention comprise a ROS-inducible regulatory region from oxyS that is operatively linked to a gene, e.g., a propionate catabolism enzyme, propionate transporter, and/or propionate binding protein gene. In the presence of ROS, e.g., H2O2, an OxyR transcription factor senses ROS and activates to the oxyS regulatory region, thereby driving expression of the operatively linked propionate catabolism enzyme, propionate transporter, and/or propionate binding protein gene and producing the propionate catabolism enzyme, propionate transporter, and/or propionate binding protein. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The genetically engineered bacteria of the invention may comprise any suitable ROS-responsive regulatory region from a gene that is activated by SoxR. Genes that are capable of being activated by SoxR are known in the art (see, e.g., Koo et al., 2003; Table 6). In certain embodiments, the genetically engineered bacteria of the invention comprise a ROS-inducible regulatory region from soxS that is operatively linked to a gene, e.g., a propionate catabolism enzyme. In the presence of ROS, the SoxR transcription factor senses ROS and activates the soxS regulatory region, thereby driving expression of the operatively linked propionate catabolism enzyme, propionate transporter, and/or propionate binding protein gene and producing a propionate catabolism enzyme, propionate transporter, and/or propionate binding protein.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H2O2 but it shows much higher reactivity for organic hydroperoxides” (Duarte et al., 2010). The genetically engineered bacteria of the invention may comprise any suitable ROS-responsive regulatory region from a gene that is repressed by OhrR. Genes that are capable of being repressed by OhrR are known in the art (see, e.g., Dubbs et al., 2012; Table 6). In certain embodiments, the genetically engineered bacteria of the invention comprise a ROS-derepressible regulatory region from ohrA that is operatively linked to a gene or gene cassette, e.g., a propionate catabolism enzyme, propionate transporter, and/or propionate binding protein gene. In the presence of ROS, e.g., NaOCl, an OhrR transcription factor senses ROS and no longer binds to the ohrA regulatory region, thereby derepressing the operatively linked propionate catabolism enzyme, propionate transporter, and/or propionate binding protein gene and producing the propionate catabolism enzyme, propionate transporter, and/or propionate binding protein.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the genetically engineered bacteria of the invention comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA” (SEQ ID NO: 312) and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The genetically engineered bacteria of the invention may comprise any suitable ROS-responsive regulatory region from a gene that is repressed by RosR. Genes that are capable of being repressed by RosR are known in the art (see, e.g., Bussmann et al., 2010; Table 6). In certain embodiments, the genetically engineered bacteria of the invention comprise a ROS-derepressible regulatory region from cgtS9 that is operatively linked to a gene or gene cassette, e.g., a propionate catabolism enzyme, propionate transporter, and/or propionate binding protein. In the presence of ROS, e.g., H2O2, a RosR transcription factor senses ROS and no longer binds to the cgtS9 regulatory region, thereby derepressing the operatively linked propionate catabolism enzyme, propionate transporter, and/or propionate binding protein gene and producing the propionate catabolism enzyme, propionate transporter, and/or propionate binding protein.

In some embodiments, it is advantageous for the genetically engineered bacteria to express a ROS-sensing transcription factor that does not regulate the expression of a significant number of native genes in the bacteria. In some embodiments, the genetically engineered bacterium of the invention expresses a ROS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the genetically engineered bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA) (SEQ ID NO: 313) residing within and near the promoter sequences of PerR-controlled genes” (Marinho et al., 2014). PerR is capable of binding a regulatory region that “overlaps part of the promoter or is immediately downstream from it” (Dubbs et al., 2012). The genetically engineered bacteria of the invention may comprise any suitable ROS-responsive regulatory region from a gene that is repressed by PerR. Genes that are capable of being repressed by PerR are known in the art (see, e.g., Dubbs et al., 2012; Table 6).

In these embodiments, the genetically engineered bacteria may comprise a two repressor activation regulatory circuit, which is used to express a propionate catabolism enzyme. The two repressor activation regulatory circuit comprises a first ROS-sensing repressor, e.g., PerR, and a second repressor, e.g., TetR, which is operatively linked to a gene or gene cassette, e.g., a propionate catabolism enzyme. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., a propionate catabolism enzyme. In the absence of PerR binding (which occurs in the absence of ROS), tetR is transcribed, and TetR represses expression of the gene or gene cassette, e.g., a propionate catabolism enzyme. In the presence of PerR binding (which occurs in the presence of ROS), tetR expression is repressed, and the gene or gene cassette, e.g a propionate catabolism enzyme, propionate transporter, and/or propionate binding protein is expressed.

A ROS-responsive transcription factor may induce, derepress, or repress gene expression depending upon the regulatory region sequence used in the genetically engineered bacteria. For example, although “OxyR is primarily thought of as a transcriptional activator under oxidizing conditions . . . . OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The genetically engineered bacteria of the invention may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 6). Or, for example, although RosR is capable of repressing a number of genes, it is also capable of activating certain genes, e.g., the narKGHJI operon. In some embodiments, the genetically engineered bacteria comprise any suitable ROS-responsive regulatory region from a gene that is activated by RosR. In addition, “PerR-mediated positive regulation has also been observed . . . and appears to involve PerR binding to distant upstream sites” (Dubbs et al., 2012). In some embodiments, the genetically engineered bacteria comprise any suitable ROS-responsive regulatory region from a gene that is activated by PerR.

One or more types of ROS-sensing transcription factors and corresponding regulatory region sequences may be present in genetically engineered bacteria. For example, “OhrR is found in both Gram-positive and Gram-negative bacteria and can coreside with either OxyR or PerR or both” (Dubbs et al., 2012). In some embodiments, the genetically engineered bacteria comprise one type of ROS-sensing transcription factor, e.g., OxyR, and one corresponding regulatory region sequence, e.g., from oxyS. In some embodiments, the genetically engineered bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the genetically engineered bacteria comprise two or more types of ROS-sensing transcription factors and one corresponding regulatory region sequence.

Nucleic acid sequences of several exemplary OxyR-regulated regulatory regions are shown in Table 7. OxyR binding sites are underlined and bolded. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 18, 19, 20, or 21, or a functional fragment thereof.

TABLE 7
Nucleotide sequences of exemplary OxyR-regulated
regulatory regions

| Regulatory | |
|---|---|
| sequence | 01234567890123456789012345678901234567890123456789 |
| katG | TGTGGCTTTTATGAAAATCACACAGTGATCACAAATTTTAAACA |
| (SEQ ID NO: | GAGCACAAAATGCTGCCTCGAAATGAGGGCGGGAAAATAAGGT |
| 18) | TATCAGCCTTGTTTTCTCCCTCATTACTTGAAGGATATGAAGCTA |
| | AAACCCTTTTTTATAAAGCATTTGTCCGAATTCGGACATAATCA |
| | AAAAAGCTTAATTAAGATCAATTTGATCTACATCTCTTTAACCA |
| | ACAATATGTAAGATCTCAACTATCGCATCCGTGGATTAATTC |
| | AATTATAACTTCTCTCTAACGCTGTGTATCGTAACGGTAACACT |
| | GTAGAGGGGAGCACATTGATGCGAATTCATTAAAGAGGAGAAA |
| | GGTACC |
| dps | TTCCGAAAATTCCTGGCGAGCAGATAAATAAGAATTGTTCTTAT |
| (SEQ ID NO: | CAATATATCTAACTCATTGAATCTTTATTAGTTTTGTTTTTCACG |
| 19) | CTTGTTACCACTATTAGTGTGATAGGAACAGCCAGAATAGCG |
| | GAACACATAGCCGGTGCTATACTTAATCTCGTTAATTACTGGGA |
| | CATAACATCAAGAGGATATGAAATTCGAATTCATTAAAGAGGA |
| | GAAAGGTACC |
| ahpC | GCTTAGATCAGGTGATTGCCCTTTGTTTATGAGGGTGTTGTAATC |
| (SEQ ID NO: | CATGTCGTTGTTGCATTTGTAAGGGCAACACCTCAGCCTGCAGG |
| 20) | CAGGCACTGAAGATACCAAAGGGTAGTTCAGATTACACGGTCA |
| | CCTGGAAAGGGGGCCATTTTACTTTTTATCGCCGCTGGCGGTGC |
| | AAAGTTCACAAAGTTGTCTTACGAAGGTTGTAAGGTAAAACTT |
| | ATCGATTTGATAATGGAAACGCATTAGCCGAATCGGCAAAAAT |
| | TGGTTACCTTACATCTCATCGAAAACACGGAGGAAGTATAGATG |
| | CGAATTCATTAAAGAGGAGAAAGGTACC |
| oxyS | CTCGAGTTCATTATCCATCCTCCATCGCCACGATAGTTCATGGC |
| (SEQ ID NO: | GATAGGTAGAATAGCAATGAACGATTATCCCTATCAAGCATTC |
| 21) | TGACTGATAATTGCTCACACGAATTCATTAAAGAGGAGAAAGGT |
| | ACC |

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the genetically engineered bacteria of the invention comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the genetically engineered bacteria of the invention comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the propionate catabolism enzyme, propionate transporter, and/or propionate binding protein in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the propionate catabolism enzyme, propionate transporter, and/or propionate binding protein in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the propionate catabolism enzyme in the presence of ROS.

In some embodiments, the gene or gene cassette for producing the propionate catabolism enzyme is present on a plasmid and operably linked to a promoter that is induced by ROS. In some embodiments, the gene or gene cassette for producing the propionate catabolism enzyme is present in the chromosome and operably linked to a promoter that is induced by ROS. In some embodiments, the gene or gene cassette for producing the propionate catabolism enzyme is present on a chromosome and operably linked to a promoter that is induced by exposure to tetracycline or arabinose. In some embodiments, the gene or gene cassette for producing the propionate catabolism enzyme, propionate transporter, and/or propionate binding protein is present on a plasmid and operably linked to a promoter that is induced by exposure to tetracycline or arabinose. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing a propionate catabolism enzyme(s), propionate transporter(s), and/or propionate binding protein(s). In some embodiments, the gene(s) capable of producing a propionate catabolism enzyme(s), propionate transporter(s), and/or propionate binding protein(s) is present on a plasmid and operatively linked to a ROS-responsive regulatory region. In some embodiments, the gene(s) capable of producing a propionate catabolism enzyme, propionate transporter, and/or propionate binding protein is present in a chromosome and operatively linked to a ROS-responsive regulatory region.

Thus, in some embodiments, the genetically engineered bacteria or genetically engineered virus produce one or more propionate catabolism enzymes under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing a propionate catabolism enzyme, propionate transporter, and/or propionate binding protein such that the propionate catabolism enzyme, propionate transporter, and/or propionate binding protein can be expressed in the host cell, and the host cell is capable of survival and/or growth in vitro, e.g., in medium, and/or in vivo. In some embodiments, a bacterium may comprise multiple copies of the gene encoding the propionate catabolism enzyme, propionate transporter, and/or propionate binding protein. In some embodiments, the gene encoding the propionate catabolism enzyme, propionate transporter, and/or propionate binding protein is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the propionate catabolism enzyme, propionate transporter, and/or propionate binding protein is expressed on a high-copy plasmid. In some embodiments, the high-copy plasmid may be useful for increasing expression of the propionate catabolism enzyme, propionate transporter, and/or propionate binding protein. In some embodiments, the gene encoding the propionate catabolism enzyme, propionate transporter, and/or propionate binding protein is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular propionate catabolism enzyme, propionate transporter, and/or propionate binding protein inserted at four different insertion sites. Alternatively, the genetically engineered bacteria may include three copies of the gene encoding a particular propionate catabolism enzyme, propionate transporter, and/or propionate binding protein inserted at three different insertion sites and three copies of the gene encoding a different propionate catabolism enzyme, propionate transporter, and/or propionate binding protein inserted at three different insertion sites.

In some embodiments, under conditions where the propionate catabolism enzyme, propionate transporter, and/or propionate binding protein is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the propionate catabolism enzyme, propionate transporter, and/or propionate binding protein and/or transcript of the gene(s) in the operon as compared to unmodified bacteria of the same subtype under the same conditions.

In some embodiments, quantitative PCR (qPCR) is used to amplify, detect, and/or quantify mRNA expression levels of the propionate catabolism enzyme, propionate transporter, and/or propionate binding protein gene(s). Primers specific for propionate catabolism enzyme, propionate transporter, and/or propionate binding protein gene(s) may be designed and used to detect mRNA in a sample according to methods known in the art. In some embodiments, a fluorophore is added to a sample reaction mixture that may contain propionate catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the propionate catabolism enzyme, propionate transporter, and/or propionate binding protein gene(s).

In some embodiments, quantitative PCR (qPCR) is used to amplify, detect, and/or quantify mRNA expression levels of the propionate catabolism enzyme, propionate transporter, and/or propionate binding protein gene(s). Primers specific for propionate catabolism enzyme, propionate transporter, and/or propionate binding protein gene(s) may be designed and used to detect mRNA in a sample according to methods known in the art. In some embodiments, a fluorophore is added to a sample reaction mixture that may contain propionate catabolism enzyme, propionate transporter, and/or propionate binding protein mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the propionate catabolism enzyme, propionate transporter, and/or propionate binding protein gene(s).

In other embodiments, the inducible promoter is a propionate responsive promoter. For example, the prpR promoter is a propionate responsive promoter. In one embodiment, the propionate responsive promoter comprises SEQ ID NO: 70.

Inducible Promoters (Nutritional and/or Chemical Inducer(s) and/or Metabolite(s))

In some embodiments, one or more gene sequence(s) encoding the propionate catabolism enzyme(s) is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding the propionate catabolism enzyme, which is induced by one or more nutritional and/or chemical inducer(s) and/or metabolite(s), such that the propionate catabolism enzyme can be expressed in the host cell, and the host cell is capable of survival and/or growth in vitro, e.g., under culture conditions, and/or in vivo, e.g., in the gut. In some embodiments, bacterial cell comprises two or more distinct propionate catabolism cassette(s), one or more of which are induced by one or more nutritional and/or chemical inducer(s) and/or metabolite(s). In some embodiments, the genetically engineered bacteria comprise multiple copies of the same propionate catabolism enzyme gene(s) and/or gene cassette(s) which are induced by one or more nutritional and/or chemical inducer(s) and/or metabolite(s). In some embodiments, the genetically engineered bacteria comprise multiple copies of different propionate catabolism enzyme genes or gene cassette(s), one or more of which are induced by one or more nutritional and/or chemical inducer(s) and/or metabolite(s).

In some embodiments, the gene encoding the propionate catabolism enzyme is present on a plasmid and operably linked to a promoter that is induced by one or more nutritional and/or chemical inducer(s) and/or metabolite(s). In some embodiments, the gene encoding the propionate catabolism enzyme is present in the chromosome and operably linked to a promoter that is induced by one or more nutritional and/or chemical inducer(s) and/or metabolite(s).

In some embodiments, the bacterial cell comprises a stably maintained plasmid or chromosome carrying the one or more gene sequences(s), inducible by one or more nutritional and/or chemical inducer(s) and/or metabolite(s), encoding a transporter of propionate and/or one or more metabolites thereof, such that the transporter can be expressed in the host cell, and the host cell is capable of survival and/or growth in vitro, e.g., in medium, and/or in vivo, e.g., in the gut. In some embodiments, bacterial cell comprises two or more distinct copies of the one or more gene sequences(s) encoding a propionate transporter, which is controlled by a promoter inducible one or more nutritional and/or chemical inducer(s) and/or metabolite(s). In some embodiments, the genetically engineered bacteria comprise multiple copies of the same one or more gene sequences(s) encoding a propionate transporter, which is controlled by a promoter inducible one or more nutritional and/or chemical inducer(s) and/or metabolite(s). In some embodiments, the one or more gene sequences(s) encoding a transporter of propionate, is present on a plasmid and operably linked to a directly or indirectly inducible promoter inducible by one or more nutritional and/or chemical inducer(s) and/or metabolite(s). In some embodiments, the one or more gene sequences(s) encoding a propionate transporter, is present on a chromosome and operably linked to a directly or indirectly inducible by one or more nutritional and/or chemical inducer(s) and/or metabolite(s).

In some embodiments, the promoter that is operably linked to the gene encoding the propionate catabolism enzyme and the promoter that is operably linked to the gene encoding the propionate transporter, is directly or indirectly induced by one or more nutritional and/or chemical inducer(s) and/or metabolite(s).

In some embodiments, one or more inducible promoter(s) are useful for or induced during in vivo expression of the one or more protein(s) of interest. In some embodiments, the promoters are induced during in vivo expression of one or more propionate catabolism enzymes and/or propionate and/or methylmalonate importers (transporters) and/or succinate exporters. In some embodiments, expression of one or more propionate catabolism enzyme(s) is driven directly or indirectly by one or more arabinose inducible promoter(s) in vivo. In some embodiments, the promoter is directly or indirectly induced by a chemical and/or nutritional inducer and/or metabolite which is co-administered with the genetically engineered bacteria of the invention.

In some embodiments, expression of one or more propionate catabolism enzyme gene(s), is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with propionate catabolism enzyme(s) and/or propionate and/or methylmalonate importer(s) (transporters) and/or succinate exporter(s) prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In some embodiments, the genetically engineered bacteria encode one or more gene sequence(s) which are inducible through an arabinose inducible system.

The genes of arabinose metabolism are organized in one operon, AraBAD, which is controlled by the PAraBAD promoter. The PAraBAD (or Para) promoter suitably fulfills the criteria of inducible expression systems. PAraBAD displays tighter control of payload gene expression than many other systems, likely due to the dual regulatory role of AraC, which functions both as an inducer and as a repressor. Additionally, the level of ParaBAD-based expression can be modulated over a wide range of L-arabinose concentrations to fine-tune levels of expression of the payload. However, the cell population exposed to sub-saturating L-arabinose concentrations is divided into two subpopulations of induced and uninduced cells, which is determined by the differences between individual cells in the availability of L-arabinose transporter (Zhang et al., Development and Application of an Arabinose-Inducible Expression System by Facilitating Inducer Uptake in Corynebacterium glutamicum; Appl. Environ. Microbiol. August 2012 vol. 78 no. 16 5831-5838). Alternatively, inducible expression from the ParaBAD can be controlled or fine-tuned through the optimization of the ribosome binding site (RBS), as described herein.

In one embodiment, expression of one or more propionate catabolism enzyme(s), e.g., one or more enzyme(s) of the MMCA and/or 2MC and/or PHA and/or MatB circuits, e.g., as described herein, is driven directly or indirectly by one or more arabinose inducible promoter(s). In one embodiment, the genetically engineered bacteria encode one or more MMCA pathway enzyme(s) whose expression is driven directly or indirectly by one or more arabinose inducible promoter(s). In one embodiment, the genetically engineered bacteria encode one or more PHA pathway enzyme(s) whose expression is driven directly or indirectly by one or more arabinose inducible promoter(s). In one embodiment, the genetically engineered bacteria encode one or more M2C pathway enzyme(s) whose expression is driven directly or indirectly by one or more arabinose inducible promoter(s). In one embodiment, the genetically engineered bacteria encode one or more MatB pathway enzyme(s) whose expression is driven directly or indirectly by one or more arabinose inducible promoter(s). In one embodiment, the genetically engineered bacteria encode one or more propionate and/or methylmalonic acid transporter(s) described herein, whose expression is driven directly or indirectly by one or more arabinose inducible promoter(s). In one embodiment, the genetically engineered bacteria encode one or more succinate exporter(s) described herein, whose expression is driven directly or indirectly by one or more arabinose inducible promoter(s).

In some embodiments, the arabinose inducible promoter is useful for or induced during in vivo expression of the one or more protein(s) of interest. In some embodiments, expression of one or more propionate catabolism enzyme(s) and/or propionate and/or methylmalonate importers (transporters) and/or succinate exporters is driven directly or indirectly by one or more arabinose inducible promoter(s) in vivo. In some embodiments, the promoter is directly or indirectly induced by a molecule (e.g., arabinose) that is co-administered with the genetically engineered bacteria of the invention.

In some embodiments, expression of one or more propionate catabolism enzyme(s) and/or propionate and/or methylmalonate importers (transporters) and/or succinate exporters, is driven directly or indirectly by one or more arabinose inducible promoter(s) during in vitro growth, preparation, or manufacturing of the strain prior to in vivo administration. In some embodiments, the arabinose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with propionate catabolism enzyme(s) prior to administration. In some embodiments, the cultures, which are induced by arabinose, are grown aerobically. In some embodiments, the cultures, which are induced by arabinose, are grown anaerobically.

In some embodiments, bacterial cell comprises two or more distinct propionate catabolism cassette(s) or other polypeptide(s) of interest, one or more of which are induced by arabinose. In some embodiments, the genetically engineered bacteria comprise multiple copies of the same propionate catabolism enzyme gene sequence(s) and/or other gene sequence(s) of interest which are induced by one or more nutritional and/or chemical inducer(s) and/or metabolite(s). In some embodiments, the genetically engineered bacteria comprise multiple copies of different propionate catabolism enzyme genes sequence(s) and/or other gene sequence(s) of interest, one or more of which are induced by one or more nutritional and/or chemical inducer(s) and/or metabolite(s).

In a first example, the arabinose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the arabinose inducible promoter drives expression under a first set of exogenous conditions, and the second promoter drives the expression under a second set of exogenous conditions. In second example, the arabinose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., arabinose and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more arabinose promoters drive expression of one or more protein(s) of interest, in combination with the FNR promoter driving the expression of the same gene sequence(s).

In some embodiments, the gene sequence(s) encoding the propionate catabolism enzyme(s) or other polypeptide(s) of interest, are present on a plasmid and operably linked to a promoter that is induced by arabinose. In some embodiments, the gene sequence(s) encoding the propionate catabolism enzyme(s) or other polypeptide(s) of interest is present in the chromosome and operably linked to a promoter that is induced by arabinose.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 142. In some embodiments, the arabinose inducible construct further comprises a gene encoding AraC, which is divergently transcribed from the same promoter as the one or more one or more propionate catabolism enzyme(s) and/or importers/transporters and/or exporters described herein. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 143. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 143.

In some embodiments, the genetically engineered bacteria comprise one or more gene sequence(s) which are inducible through a rhamnose inducible system. The genes rhaBAD are organized in one operon which is controlled by the rhaP BAD promoter. The rhaP BAD promoter is regulated by two activators, RhaS and RhaR, and the corresponding genes belong to one transcription unit which divergently transcribed in the opposite direction of rhaBAD. In the presence of L-rhamnose, RhaR binds to the rhaP RS promoter and activates the production of RhaR and RhaS. RhaS together with L-rhamnose then bind to the rhaP BAD and the rhaP T promoter and activate the transcription of the structural genes. In contrast to the arabinose system, in which AraC is provided and divergently transcribed in the gene sequence(s), it is not necessary to express the regulatory proteins in larger quantities in the rhamnose expression system because the amounts expressed from the chromosome are sufficient to activate transcription even on multi-copy plasmids. Therefore, only the rhaP BAD promoter is cloned upstream of the gene that is to be expressed. Full induction of rhaBAD transcription also requires binding of the CRP-cAMP complex, which is a key regulator of catabolite repression. Alternatively, inducible expression from the rhaBAD can be controlled or fine-tuned through the optimization of the ribosome binding site (RBS), as described herein.

In one embodiment, expression of one or more propionate catabolism enzyme(s), e.g., one or more enzyme(s) of the MMCA and/or 2MC and/or PHA and/or MatB circuits, e.g., as described herein, is driven directly or indirectly by one or more rhamnose inducible promoter(s). In one embodiment, the genetically engineered bacteria encode one or more MMCA pathway enzyme(s) whose expression is driven directly or indirectly by one or more rhamnose inducible promoter(s). In one embodiment, the genetically engineered bacteria encode one or more PHA pathway enzyme(s) whose expression is driven directly or indirectly by one or more rhamnose inducible promoter(s). In one embodiment, the genetically engineered bacteria encode one or more M2C pathway enzyme(s) whose expression is driven directly or indirectly by one or more rhamnose inducible promoter(s). In one embodiment, the genetically engineered bacteria encode one or more MatB pathway enzyme(s) whose expression is driven directly or indirectly by one or more rhamnose inducible promoter(s). In one embodiment, the genetically engineered bacteria encode one or more propionate and/or methylmalonic acid transporter(s) described herein, whose expression is driven directly or indirectly by one or more rhamnose inducible promoter(s). In one embodiment, the genetically engineered bacteria encode one or more succinate exporter(s) described herein, whose expression is driven directly or indirectly by one or more rhamnose inducible promoter(s).

In some embodiments, the rhamnose inducible promoter is useful for or induced during in vivo expression of the one or more protein(s) of interest. In some embodiments, expression of one or more propionate catabolism enzyme(s) and/or propionate and/or methylmalonate importers (transporters) and/or succinate exporters is driven directly or indirectly by one or more rhamnose inducible promoter(s) in vivo. In some embodiments, the promoter is directly or indirectly induced by a molecule (e.g., rhamnose) that is co-administered with the genetically engineered bacteria of the invention.

In some embodiments, expression of one or more propionate catabolism enzyme(s) and/or propionate and/or methylmalonate importers (transporters) and/or succinate exporters, is driven directly or indirectly by one or more rhamnose inducible promoter(s) during in vitro growth, preparation, or manufacturing of the strain prior to in vivo administration. In some embodiments, the rhamnose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e g, rhamnose, that is added to in the bacterial culture to induce expression and pre-load the bacterium with propionate catabolism enzyme(s) prior to administration. In some embodiments, the cultures, which are induced by rhamnose, are grown aerobically. In some embodiments, the cultures, which are induced by rhamnose, are grown anaerobically.

In some embodiments, bacterial cell comprises two or more distinct propionate catabolism cassette(s) or other polypeptide(s) of interest, one or more of which are induced by rhamnose. In some embodiments, the genetically engineered bacteria comprise multiple copies of the same propionate catabolism enzyme gene sequence(s) and/or other gene sequence(s) of interest which are induced by rhamnose. In some embodiments, the genetically engineered bacteria comprise multiple copies of different propionate catabolism enzyme genes sequence(s) and/or other gene sequence(s) of interest, one or more of which are induced by rhamnose.

In a first example, the rhamnose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the rhamnose inducible promoter drives expression under a first set of exogenous conditions, and the second promoter drives the expression under a second set of exogenous conditions. In second example, the rhamnose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., rhamnose and IPTG). In another non-limiting example, the first inducing conditions are culture conditions, e.g., the presence of rhamnose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more rhamnose promoters drive expression of one or more protein(s) of interest, in combination with the FNR promoter driving the expression of the same gene sequence(s).

In some embodiments, the gene sequence(s) encoding the propionate catabolism enzyme(s) or other polypeptide(s) of interest, are present on a plasmid and operably linked to a promoter that is induced by rhamnose. In some embodiments, the gene sequence(s) encoding the propionate catabolism enzyme(s) or other polypeptide(s) of interest is present in the chromosome and operably linked to a promoter that is induced by rhamnose.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 145.

In some embodiments, the genetically engineered bacteria comprise one or more gene sequence(s) which are inducible through an Isopropyl β-D-1-thiogalactopyranoside (IPTG) inducible system or other compound which induced transcription from the Lac Promoter. IPTG is a molecular mimic of allolactose, a lactose metabolite that activates transcription of the lac operon. In contrast to allolactose, the sulfur atom in IPTG creates a non-hydrolyzable chemical blond, which prevents the degradation of IPTG, allowing the concentration to remain constant. IPTG binds to the lac repressor and releases the tetrameric repressor (LacI) from the lac operator in an allosteric manner, thereby allowing the transcription of genes in the lac operon. Since IPTG is not metabolized by E. coli, its concentration stays constant and the rate of expression of Lac promoter-controlled is tightly controlled, both in vivo and in vitro. IPTG intake is independent on the action of lactose permease, since other transport pathways are also involved. Inducible expression from the PLac can be controlled or fine-tuned through the optimization of the ribosome binding site (RBS), as described herein. Other compounds which inactivate LacI, can be used instead of IPTG in a similar manner.

In one embodiment, expression of one or more propionate catabolism enzyme(s), e.g., one or more enzyme(s) of the MMCA and/or 2MC and/or PHA and/or MatB circuits, e.g., as described herein, is driven directly or indirectly by one or more IPTG inducible promoter(s). In one embodiment, the genetically engineered bacteria encode one or more MMCA pathway enzyme(s) whose expression is driven directly or indirectly by one or more IPTG inducible promoter(s). In one embodiment, the genetically engineered bacteria encode one or more PHA pathway enzyme(s) whose expression is driven directly or indirectly by one or more IPTG inducible promoter(s). In one embodiment, the genetically engineered bacteria encode one or more M2C pathway enzyme(s) whose expression is driven directly or indirectly by one or more IPTG inducible promoter(s). In one embodiment, the genetically engineered bacteria encode one or more MatB pathway enzyme(s) whose expression is driven directly or indirectly by one or more IPTG inducible promoter(s). In one embodiment, the genetically engineered bacteria encode one or more propionate and/or methylmalonic acid transporter(s) described herein, whose expression is driven directly or indirectly by one or more IPTG inducible promoter(s). In one embodiment, the genetically engineered bacteria encode one or more succinate exporter(s) described herein, whose expression is driven directly or indirectly by one or more IPTG inducible promoter(s).

In some embodiments, the IPTG inducible promoter is useful for or induced during in vivo expression of the one or more protein(s) of interest. In some embodiments, expression of one or more propionate catabolism enzyme(s) and/or propionate and/or methylmalonate importers (transporters) and/or succinate exporters is driven directly or indirectly by one or more IPTG inducible promoter(s) in vivo. In some embodiments, the promoter is directly or indirectly induced by a molecule (e.g., IPTG) that is co-administered with the genetically engineered bacteria of the invention.

In some embodiments, expression of one or more propionate catabolism enzyme(s) and/or propionate and/or methylmalonate importers (transporters) and/or succinate exporters, is driven directly or indirectly by one or more IPTG inducible promoter(s) during in vitro growth, preparation, or manufacturing of the strain prior to in vivo administration. In some embodiments, the IPTG inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., IPTG, that is added to in the bacterial culture to induce expression and pre-load the bacterium with propionate catabolism enzyme(s) prior to administration. In some embodiments, the cultures, which are induced by IPTG, are grown aerobically. In some embodiments, the cultures, which are induced by IPTG, are grown anaerobically.

In some embodiments, bacterial cell comprises two or more distinct propionate catabolism cassette(s) or other polypeptide(s) of interest, one or more of which are induced by IPTG. In some embodiments, the genetically engineered bacteria comprise multiple copies of the same propionate catabolism enzyme gene sequence(s) and/or other gene sequence(s) of interest which are induced IPTG. In some embodiments, the genetically engineered bacteria comprise multiple copies of different propionate catabolism enzyme genes sequence(s) and/or other gene sequence(s) of interest, one or more of which are induced by IPTG.

In a first example, the IPTG inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the IPTG inducible promoter drives expression under a first set of exogenous conditions, and the second promoter drives the expression under a second set of exogenous conditions. In second example, the IPTG promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., IPTG and IPTG). In another non-limiting example, the first inducing conditions are culture conditions, e.g., the presence of IPTG, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more IPTG promoters drive expression of one or more protein(s) of interest, in combination with the FNR promoter driving the expression of the same gene sequence(s).

In some embodiments, the gene sequence(s) encoding the propionate catabolism enzyme(s) or other polypeptide(s) of interest, are present on a plasmid and operably linked to a promoter that is induced by IPTG. In some embodiments, the gene sequence(s) encoding the propionate catabolism enzyme(s) or other polypeptide(s) of interest is present in the chromosome and operably linked to a promoter that is induced by IPTG.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO:146. In some embodiments, the IPTG inducible construct further comprises a gene encoding which is divergently transcribed from the same promoter as the one or more one or more propionate catabolism enzyme(s) and/or importers/transporters and/or exporters described herein. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 148. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 148.

In some embodiments, the genetically engineered bacteria comprise one or more gene sequence(s) which are inducible through a tetracycline inducible system. The initial system Gossen and Bujard (Tight control of gene expression in mammalian cells by tetracycline-responsive promoters. Gossen M Bujard H. PNAS, 1992 Jun. 15; 89(12):5547-51) developed is known as tetracycline off: in the presence of tetracycline, expression from a tet-inducible promoter is reduced. Tetracycline-controlled transactivator (tTA) was created by fusing tetR with the C-terminal domain of VP16 (virion protein 16) from herpes simplex virus. In the absence of tetracycline, the tetR portion of tTA will bind tetO sequences in the tet promoter, and the activation domain promotes expression. In the presence of tetracycline, tetracycline binds to tetR, precluding tTA from binding to the tetO sequences. Next, a reverse Tet repressor (rTetR), was developed which created a reliance on the presence of tetracycline for induction, rather than repression. The new transactivator rtTA (reverse tetracycline-controlled transactivator) was created by fusing rTetR with VP16. The tetracycline on system is also known as the rtTA-dependent system.

In one embodiment, expression of one or more propionate catabolism enzyme(s), e.g., one or more enzyme(s) of the MMCA and/or 2MC and/or PHA and/or MatB circuits, e.g., as described herein, is driven directly or indirectly by one or more tetracycline inducible promoter(s). In one embodiment, the genetically engineered bacteria encode one or more MMCA pathway enzyme(s) whose expression is driven directly or indirectly by one or more tetracycline inducible promoter(s). In one embodiment, the genetically engineered bacteria encode one or more PHA pathway enzyme(s) whose expression is driven directly or indirectly by one or more tetracycline inducible promoter(s). In one embodiment, the genetically engineered bacteria encode one or more M2C pathway enzyme(s) whose expression is driven directly or indirectly by one or more tetracycline inducible promoter(s). In one embodiment, the genetically engineered bacteria encode one or more MatB pathway enzyme(s) whose expression is driven directly or indirectly by one or more tetracycline inducible promoter(s). In one embodiment, the genetically engineered bacteria encode one or more propionate and/or methylmalonic acid transporter(s) described herein, whose expression is driven directly or indirectly by one or more tetracycline inducible promoter(s). In one embodiment, the genetically engineered bacteria encode one or more succinate exporter(s) described herein, whose expression is driven directly or indirectly by one or more tetracycline inducible promoter(s).

In some embodiments, the tetracycline inducible promoter is useful for or induced during in vivo expression of the one or more protein(s) of interest. In some embodiments, expression of one or more propionate catabolism enzyme(s) and/or propionate and/or methylmalonate importers (transporters) and/or succinate exporters is driven directly or indirectly by one or more tetracycline inducible promoter(s) in vivo. In some embodiments, the promoter is directly or indirectly induced by a molecule (e.g., tetracycline) that is co-administered with the genetically engineered bacteria of the invention.

In some embodiments, expression of one or more propionate catabolism enzyme(s) and/or propionate and/or methylmalonate importers (transporters) and/or succinate exporters, is driven directly or indirectly by one or more tetracycline inducible promoter(s) during in vitro growth, preparation, or manufacturing of the strain prior to in vivo administration. In some embodiments, the tetracycline inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., tetracycline, that is added to in the bacterial culture to induce expression and pre-load the bacterium with propionate catabolism enzyme(s) prior to administration. In some embodiments, the cultures, which are induced by tetracycline, are grown aerobically. In some embodiments, the cultures, which are induced by tetracycline, are grown anaerobically.

In some embodiments, bacterial cell comprises two or more distinct propionate catabolism cassette(s) or other polypeptide(s) of interest, one or more of which are induced by tetracycline. In some embodiments, the genetically engineered bacteria comprise multiple copies of the same propionate catabolism enzyme gene sequence(s) and/or other gene sequence(s) of interest which are induced by tetracycline. In some embodiments, the genetically engineered bacteria comprise multiple copies of different propionate catabolism enzyme genes sequence(s) and/or other gene sequence(s) of interest, one or more of which are induced by tetracycline.

In a first example, the tetracycline inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the tetracycline inducible promoter drives expression under a first set of exogenous conditions, and the second promoter drives the expression under a second set of exogenous conditions. In second example, the tetracycline promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., tetracycline and IPTG). In another non-limiting example, the first inducing conditions are culture conditions, e.g., the presence of tetracycline, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more tetracycline promoters drive expression of one or more protein(s) of interest, in combination with the FNR promoter driving the expression of the same gene sequence(s).

In some embodiments, the gene sequence(s) encoding the propionate catabolism enzyme(s) or other polypeptide(s) of interest, are present on a plasmid and operably linked to a promoter that is induced by tetracycline. In some embodiments, the gene sequence(s) encoding the propionate catabolism enzyme(s) or other polypeptide(s) of interest is present in the chromosome and operably linked to a promoter that is induced by tetracycline.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the bolded sequences of SEQ ID NO: 320 (tet promoter is in bold). In some embodiments, the tetracycline inducible construct further comprises a gene encoding AraC, which is divergently transcribed from the same promoter as the one or more one or more propionate catabolism enzyme(s) and/or importers/transporters and/or exporters described herein. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 320 in italics (Tet repressor is in italics). In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 320 in italics (Tet repressor is in italics).

In some embodiments, the genetically engineered bacteria comprise one or more gene sequence(s) whose expression is controlled by a temperature sensitive mechanism. Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun. 10; 203: 32-40, and references therein). Thermoregulated protein expression using the mutant cI857 repressor and the pL and/or pR phage λ promoters have been used to engineer recombinant bacterial strains. The gene of interest cloned downstream of the λ promoters can then be efficiently regulated by the mutant thermolabile cI857 repressor of bacteriophage λ. At temperatures below 37° C., cI857 binds to the oL or oR regions of the pR promoter and blocks transcription by RNA polymerase. At higher temperatures, the functional cI857 dimer is destabilized, binding to the oL or oR DNA sequences is abrogated, and mRNA transcription is initiated. Inducible expression from the thermoregulated promoter can be controlled or further fine-tuned through the optimization of the ribosome binding site (RBS), as described herein.

In one embodiment, expression of one or more protein(s) of interest is driven directly or indirectly by one or more thermoregulated promoter(s). In one embodiment, expression of one or more propionate catabolism enzyme(s), e.g., one or more enzyme(s) of the MMCA and/or 2MC and/or PHA and/or MatB circuits, e.g., as described herein, is driven directly or indirectly by one or more thermoregulated inducible promoter(s). In one embodiment, the genetically engineered bacteria encode one or more MMCA pathway enzyme(s) whose expression is driven directly or indirectly by one or more thermoregulated inducible promoter(s). In one embodiment, the genetically engineered bacteria encode one or more PHA pathway enzyme(s) whose expression is driven directly or indirectly by one or more thermoregulated inducible promoter(s). In one embodiment, the genetically engineered bacteria encode one or more M2C pathway enzyme(s) whose expression is driven directly or indirectly by one or more thermoregulated inducible promoter(s). In one embodiment, the genetically engineered bacteria encode one or more MatB pathway enzyme(s) whose expression is driven directly or indirectly by one or more thermoregulated inducible promoter(s). In one embodiment, the genetically engineered bacteria encode one or more propionate and/or methylmalonic acid transporter(s) described herein, whose expression is driven directly or indirectly by one or more thermoregulated inducible promoter(s). In one embodiment, the genetically engineered bacteria encode one or more succinate exporter(s) described herein, whose expression is driven directly or indirectly by one or more thermoregulated inducible promoter(s).

In some embodiments, the thermoregulated promoter is useful for or induced during in vivo expression of the one or more protein(s) of interest. In some embodiments, expression of one or more propionate catabolism enzyme(s) and/or other protein(s) of interest is driven directly or indirectly by one or more thermoregulated promoter(s) in vivo.

In some embodiments, expression of one or more protein(s) of interest is driven directly or indirectly by one or more thermoregulated promoter(s) during in vitro growth, preparation, or manufacturing of the strain prior to in vivo administration. In some embodiments, it may be advantageous to shut off production of the one or more propionate catabolism enzyme(s) and/or other protein(s) of interest. This can be done in a thermoregulated system by growing the strain at lower temperatures, e.g., 30 C. Expression can then be induced by elevating the temperature to 37 C and/or 42 C. In some embodiments, the thermoregulated promoter(s) are induced in culture, e.g., grown in a flask, fermenter or other appropriate culture vessel, e.g., used during cell growth, cell expansion, fermentation, recovery, purification, formulation, and/or manufacture. In some embodiments, the cultures, which are induced by temperatures between 37 C and 42 C, are grown aerobically. In some embodiments, the cultures, which are induced by induced by temperatures between 37 C and 42 C, are grown anaerobically.

In some embodiments, bacterial cell comprises two or more distinct propionate catabolism cassette(s) or other polypeptide(s) of interest, one or more of which are induced by temperature. In some embodiments, the genetically engineered bacteria comprise multiple copies of the same propionate catabolism enzyme gene sequence(s) and/or other gene sequence(s) of interest which are induced by temperature. In some embodiments, the genetically engineered bacteria comprise multiple copies of different propionate catabolism enzyme genes sequence(s) and/or other gene sequence(s) of interest, one or more of which are induced by temperature.

In a first example, the temperature inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the temperature inducible promoter drives expression under a first set of exogenous conditions, and the second promoter drives the expression under a second set of exogenous conditions. In second example, the temperature promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., temperature regulation and IPTG). In another non-limiting example, the first inducing conditions are culture conditions, e.g., the permissive temperature, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more temperature regulated promoters drive expression of one or more protein(s) of interest, in combination with the FNR promoter driving the expression of the same gene sequence(s).

In some embodiments, the gene sequence(s) encoding the propionate catabolism enzyme(s) or other polypeptide(s) of interest, are present on a plasmid and operably linked to a promoter that is induced by temperature. In some embodiments, the gene sequence(s) encoding the propionate catabolism enzyme(s) or other polypeptide(s) of interest is present in the chromosome and operably linked to a promoter that is induced by temperature.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 150. In some embodiments, the thermoregulated construct further comprises a gene encoding mutant cI857 repressor, which is divergently transcribed from the same promoter as the one or more one or more propionate catabolism enzyme(s) and/or importers/transporters and/or exporters described herein. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 151. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 151.

In some embodiments, the genetically engineered bacteria comprise one or more gene sequence(s) which are indirectly inducible through a system driven by the PssB promoter. The PssB promoter is active under aerobic conditions, and shuts off under anaerobic conditions.

This promoter can be used to express a gene of interest under aerobic conditions. This promoter can also be used to tightly control the expression of a gene product such that it is only expressed under anaerobic conditions. In this case, the oxygen induced PssB promoter induces the expression of a repressor, which represses the expression of a gene of interest. As a result, the gene of interest is only expressed in the absence of the repressor, i.e., under anaerobic conditions. This strategy has the advantage of an additional level of control for improved fine-tuning and tighter control. FIG. 40A depicts a schematic of the gene organization of a PssB promoter.

In one embodiment, expression of one or more propionate catabolism enzyme(s), e.g., one or more enzyme(s) of the MMCA and/or 2MC and/or PHA and/or MatB circuits, e.g., as described herein, is driven directly or indirectly by one or more PssB promoter(s). In one embodiment, the genetically engineered bacteria encode one or more MMCA pathway enzyme(s) whose expression is driven directly or indirectly by one or more PssB promoter(s). In one embodiment, the genetically engineered bacteria encode one or more PHA pathway enzyme(s) whose expression is driven directly or indirectly by one or more PssB promoter(s). In one embodiment, the genetically engineered bacteria encode one or more M2C pathway enzyme(s) whose expression is driven directly or indirectly by one or more PssB promoter(s). In one embodiment, the genetically engineered bacteria encode one or more MatB pathway enzyme(s) whose expression is driven directly or indirectly by one or more PssB promoter(s). In one embodiment, the genetically engineered bacteria encode one or more propionate and/or methylmalonic acid transporter(s) described herein, whose expression is driven directly or indirectly by one or more PssB promoter(s). In one embodiment, the genetically engineered bacteria encode one or more succinate exporter(s) described herein, whose expression is driven directly or indirectly by one or more PssB promoter(s).

In some embodiments, the PssB promoter is useful for or induced during in vivo expression of the one or more protein(s) of interest. In some embodiments, expression of one or more propionate catabolism enzyme(s) and/or propionate and/or methylmalonate importers (transporters) and/or succinate exporters is driven directly or indirectly by one or more PssB promoter(s) in vivo. In some embodiments, the promoter is directly or indirectly induced by a molecule (e.g., arabinose) that is co-administered with the genetically engineered bacteria of the invention.

In some embodiments, expression of one or more propionate catabolism enzyme(s) and/or propionate and/or methylmalonate importers (transporters) and/or succinate exporters, is driven directly or indirectly by one or more PssB promoter(s) during in vitro growth, preparation, or manufacturing of the strain prior to in vivo administration. In some embodiments, the PssB promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with propionate catabolism enzyme(s) prior to administration. In some embodiments, the cultures, which are induced by arabinose, are grown aerobically. In some embodiments, the cultures, which are induced by arabinose, are grown anaerobically.

In some embodiments, bacterial cell comprises two or more distinct propionate catabolism cassette(s) or other polypeptide(s) of interest, one or more of which are induced by arabinose. In some embodiments, the genetically engineered bacteria comprise multiple copies of the same propionate catabolism enzyme gene sequence(s) and/or other gene sequence(s) of interest which are induced by one or more nutritional and/or chemical inducer(s) and/or metabolite(s). In some embodiments, the genetically engineered bacteria comprise multiple copies of different propionate catabolism enzyme genes sequence(s) and/or other gene sequence(s) of interest, one or more of which are induced by one or more nutritional and/or chemical inducer(s) and/or metabolite(s).

In a first example, the PssB promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the PssB promoter drives expression under a first set of exogenous conditions, and the second promoter drives the expression under a second set of exogenous conditions. In second example, the PssB promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., PssB and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more PssB promoters drive expression of one or more protein(s) of interest, in combination with the FNR promoter driving the expression of the same gene sequence(s).

In some embodiments, the gene sequence(s) encoding the propionate catabolism enzyme(s) or other polypeptide(s) of interest, are present on a plasmid and operably linked to a promoter that is induced by arabinose. In some embodiments, the gene sequence(s) encoding the propionate catabolism enzyme(s) or other polypeptide(s) of interest is present in the chromosome and operably linked to a promoter that is induced by arabinose.

In another non-limiting example, this strategy can be used to control expression of thyA and/or dapA, e.g., to make a conditional auxotroph. The chromosomal copy of dapA or ThyA is knocked out. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic conditions, e.g., the gut, but prevent survival under aerobic conditions (biosafety switch). In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 321.

Constitutive Promoters

In some embodiments, the gene encoding the payload is present on a plasmid and operably linked to a constitutive promoter. In some embodiments, the gene encoding the payload is present on a chromosome and operably linked to a constitutive promoter.

In some embodiments, the constitutive promoter is active under in vivo conditions, e.g., the gut, or in the presence of metabolites associated with certain diseases, such as PA and/or MMA, as described herein. In some embodiments, the promoters are active under in vitro conditions, e.g., various cell culture and/or cell manufacturing conditions, as described herein. In some embodiments, the constitutive promoter is active under in vivo conditions, e.g., the gut and/or in the presence of metabolites associated with certain diseases, such as PA and/or MMA, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions).

In some embodiments, the constitutive promoter is active in exogenous environmental conditions specific to the gut of a mammal. In some embodiments, the constitutive promoter is active in exogenous environmental conditions specific to the small intestine of a mammal. In some embodiments, the constitutive promoter is active in low-oxygen or anaerobic conditions such as the environment of the mammalian gut. In some embodiments, the constitutive promoter is active in the presence of molecules or metabolites that are specific to the gut of a mammal. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions.

Bacterial constitutive promoters are known in the art. Exemplary constitutive promoters are listed in the following Tables. The strength of the constitutive promoter can be further fine-tuned through the selection of ribosome binding sites of the desired strengths.

In some embodiments, the gene sequence(s) encoding a propionate catabolism enzyme is operably linked to an Escherichia coli σ70 promoter. Exemplary E. coli σ70 promoters are listed in Table 8.

In some embodiments, the gene sequence(s) encoding a propionate catabolism enzyme is operably linked to an Escherichia coli σ70 promoter. Exemplary E. coli σ70 promoters are listed in Table 6A.

TABLE 8
Constitutive E. coli σ70 promoters

| SEQ | Name | Description | Promoter Sequence | Length |
|---|---|---|---|---|
| SEQ ID NO: | BBa_I14018 | P(Bla) | ... | 35 |
| 152 | | | gtttatacataggcgagtactctgttatgg | |
| SEQ ID NO: | BBa_I14033 | P(Cat) | ... | 38 |
| 153 | | | agaggaccaactttcaccataatgaaaca | |
| SEQ ID NO: | BBa_I14034 | P(Kat) | ... | 45 |
| 154 | | | taaacaactaacggacaattctacctaaca | |
| SEQ ID NO: | BBa_I732021 | Template for Building | ... | 159 |
| 155 | | Primer Family Member | acatcaagccaaattaaacaggattaacac | |
| SEQ ID NO: | BBa_I742126 | Reverse lambda cI- | ... | 49 |
| 156 | | regulated promoter | gaggtaaaatagtcaacacgcacggtgtta | |
| SEQ ID NO: | BBa_J01006 | Key Promoter absorbs 3 | ... | 59 |
| 157 | | | caggccggaataactccctataatgcgcca | |
| SEQ ID NO: | BBa_J23100 | constitutive promoter | ... | 35 |
| 158 | | family member | ggctagctcagtcctaggtacagtgctagc | |
| SEQ ID NO: | BBa_J23101 | constitutive promoter | ... | 35 |
| 159 | | family member | agctagctcagtcctaggtattatgctagc | |
| SEQ ID NO: | BBa_J23102 | constitutive promoter | ... | 35 |
| 160 | | family member | agctagctcagtcctaggtactgtgctagc | |
| SEQ ID NO: | BBa_J23103 | constitutive promoter | ... | 35 |
| 161 | | family member | agctagctcagtcctagggattatgctagc | |
| SEQ ID NO: | BBa_J23104 | constitutive promoter | ... | 35 |
| 162 | | family member | agctagctcagtcctaggtattgtgctagc | |
| SEQ ID NO: | BBa_J23105 | constitutive promoter | ... | 35 |
| 163 | | family member | ggctagctcagtcctaggtactatgctagc | |
| SEQ ID NO: | BBa_J23106 | constitutive promoter | ... | 35 |
| 164 | | family member | ggctagctcagtcctaggtatagtgctagc | |
| SEQ ID NO: | BBa_J23107 | constitutive promoter | ... | 35 |
| 165 | | family member | ggctagctcagccctaggtattatgctagc | |
| SEQ ID NO: | BBa_J23108 | constitutive promoter | ... | 35 |
| 166 | | family member | agctagctcagtcctaggtataatgctagc | |
| SEQ ID NO: | BBa_J23109 | constitutive promoter | ... | 35 |
| 167 | | family member | agctagctcagtcctagggactgtgctagc | |
| SEQ ID NO: | BBa_J23110 | constitutive promoter | ... | 35 |
| 168 | | family member | ggctagctcagtcctaggtacaatgctagc | |
| SEQ ID NO: | BBa_J23111 | constitutive promoter | ... | 35 |
| 169 | | family member | ggctagctcagtcctaggtatagtgctagc | |
| SEQ ID NO: | BBa_J23112 | constitutive promoter | ... | 35 |
| 170 | | family member | agctagctcagtcctagggattatgctagc | |
| SEQ ID NO: | BBa_J23113 | constitutive promoter | ... | 35 |
| 171 | | family member | ggctagctcagtcctagggattatgctagc | |
| SEQ ID NO: | BBa_J23114 | constitutive promoter | ... | 35 |
| 172 | | family member | ggctagctcagtcctaggtacaatgctagc | |
| SEQ ID NO: | BBa_J23115 | constitutive promoter | ... | 35 |
| 173 | | family member | agctagctcagcccttggtacaatgctagc | |
| SEQ ID NO: | BBa_J23116 | constitutive promoter | ... | 35 |
| 174 | | family member | agctagctcagtcctagggactatgctagc | |
| SEQ ID NO: | BBa_J23117 | constitutive promoter | ... | 35 |
| 175 | | family member | agctagctcagtcctagggattgtgctagc | |
| SEQ ID NO: | BBa_J23118 | constitutive promoter | ... | 35 |
| 176 | | family member | ggctagctcagtcctaggtattgtgctagc | |
| SEQ ID NO: | BBa_J23119 | constitutive promoter | ... | 35 |
| 177 | | family member | agctagctcagtcctaggtataatgctagc | |
| SEQ ID NO: | BBa_J23150 | 1 bp mutant from J23107 | ... | 35 |
| 178 | | | ggctagctcagtcctaggtattatgctagc | |
| SEQ ID NO: | BBa_J23151 | 1 bp mutant from J23114 | ... | 35 |
| 179 | | | ggctagctcagtcctaggtacaatgctagc | |
| SEQ ID NO: | BBa_J44002 | pBAD reverse | ... | 130 |
| 180 | | | aaagtgtgacgccgtgcaaataatcaatgt | |
| SEQ ID NO: | BBa_J48104 | NikR promoter, a protein | ... | 40 |
| 181 | | of the ribbon helix-helix | gacgaatacttaaaatcgtcatacttattt | |
| | | family of trancription | | |
| | | factors that repress expre | | |
| SEQ ID NO: | BBa_J54200 | lacq_Promoter | ... | 50 |
| 182 | | | aaacctttcgcggtatggcatgatagcgcc | |
| SEQ ID NO: | BBa_J56015 | lacIQ - promoter sequence | ... | 57 |
| 183 | | | tgatagcgcccggaagagagtcaattcagg | |
| SEQ ID NO: | BBa_J64951 | E. Coli CreABCD | ... | 81 |
| 184 | | phosphate sensing operon | ttatttaccgtgacgaactaattgctcgtg | |
| | | promoter | | |
| SEQ ID NO: | BBa_K088007 | GlnRS promoter | ... | 38 |
| 185 | | | catacgccgttatacgttgtttacgctttg | |
| SEQ ID NO: | BBa_K119000 | Constitutive weak | ... | 38 |
| 186 | | promoter of lacZ | ttatgcttccggctcgtatgttgtgtggac | |
| SEQ ID NO: | BBa_K119001 | Mutated LacZ promoter | ... | 38 |
| 187 | | | ttatgcttccggctcgtatggtgtgtggac | |
| SEQ ID NO: | BBa_K1330002 | Constitutive promoter | ... | 35 |
| 188 | | (J23105) | ggctagctcagtcctaggtactatgctagc | |
| SEQ ID NO: | BBa_K137029 | constitutive promoter with | ...atatatatatatatataatggaagcgtttt | 39 |
| 189 | | (TA)10 between −10 and −35 | | |
| | | elements | | |
| SEQ ID NO: | BBa_K137030 | constitutive promoter with | ...atatatatatatatataatggaagcgtttt | 37 |
| 190 | | (TA)9 between −10 and −35 | | |
| | | elements | | |
| SEQ ID NO: | BBa_K137031 | constitutive promoter with | ... | 62 |
| 191 | | (C)10 between −10 and −35 | ccccgaaagcttaagaatataattgtaagc | |
| | | elements | | |
| SEQ ID NO: | BBa_K137032 | constitutive promoter with | ... | 64 |
| 192 | | (C)12 between −10 and −35 | ccccgaaagcttaagaatataattgtaagc | |
| | | elements | | |
| SEQ ID NO: | BBa_K137085 | optimized (TA) repeat | ... | 31 |
| 193 | | constitutive promoter with | tgacaatatatatatatatataatgctagc | |
| | | 13 bp between −10 and −35 | | |
| | | elements | | |
| SEQ ID NO: | BBa_K137086 | optimized (TA) repeat | ... | 33 |
| 194 | | constitutive promoter with | acaatatatatatatatatataatgctagc | |
| | | 15 bp between −10 and −35 | | |
| | | elements | | |
| SEQ ID NO: | BBa_K137087 | optimized (TA) repeat | ...aatatatatatatatatatataatgctagc | 35 |
| 195 | | constitutive promoter with | | |
| | | 17 bp between −10 and −35 | | |
| | | elements | | |
| SEQ ID NO: | BBa_K137088 | optimized (TA) repeat | ...tatatatatatatatatatataatgctagc | 37 |
| 196 | | constitutive promoter with | | |
| | | 19 bp between −10 and −35 | | |
| | | elements | | |
| SEQ ID NO: | BBa_K137089 | optimized (TA) repeat | ...tatatatatatatatatatataatgctagc | 39 |
| 197 | | constitutive promoter with | | |
| | | 21 bp between −10 and −35 | | |
| | | elements | | |
| SEQ ID NO: | BBa_K137090 | optimized (A) repeat | ... | 35 |
| 198 | | constitutive promoter with | aaaaaaaaaaaaaaaaaatataatgctagc | |
| | | 17 bp between −10 and −35 | | |
| | | elements | | |
| SEQ ID NO: | BBa_K137091 | optimized (A) repeat | ... | 36 |
| 199 | | constitutive promoter with | aaaaaaaaaaaaaaaaaatataatgctagc | |
| | | 18 bp between −10 and −35 | | |
| | | elements | | |
| SEQ ID NO: | BBa_K1585100 | Anderson Promoter with | ... | 78 |
| 200 | | lacI binding site | ggaattgtgagcggataacaatttcacaca | |
| SEQ ID NO: | BBa_K1585101 | Anderson Promoter with | ... | 78 |
| 201 | | lacI binding site | ggaattgtgagcggataacaatttcacaca | |
| SEQ ID NO: | BBa_K1585102 | Anderson Promoter with | ... | 78 |
| 202 | | lacI binding site | ggaattgtgagcggataacaatttcacaca | |
| SEQ ID NO: | BBa_K1585103 | Anderson Promoter with | ... | 78 |
| 203 | | lacI binding site | ggaattgtgagcggataacaatttcacaca | |
| SEQ ID NO: | BBa_K1585104 | Anderson Promoter with | ... | 78 |
| 204 | | lacI binding site | ggaattgtgagcggataacaatttcacaca | |
| SEQ ID NO: | BBa_K1585105 | Anderson Promoter with | ... | 78 |
| 205 | | lacI binding site | ggaattgtgagcggataacaatttcacaca | |
| SEQ ID NO: | BBa_K1585106 | Anderson Promoter with | ... | 78 |
| 206 | | lacI binding site | ggaattgtgagcggataacaatttcacaca | |
| SEQ ID NO: | BBa_K1585110 | Anderson Promoter with | ... | 78 |
| 207 | | lacI binding site | ggaattgtgagcggataacaatttcacaca | |
| SEQ ID NO: | BBa_K1585113 | Anderson Promoter with | ... | 78 |
| 208 | | lacI binding site | ggaattgtgagcggataacaatttcacaca | |
| SEQ ID NO: | BBa_K1585115 | Anderson Promoter with | ... | 78 |
| 209 | | lacI binding site | ggaattgtgagcggataacaatttcacaca | |
| SEQ ID NO: | BBa_K1585116 | Anderson Promoter with | ... | 78 |
| 210 | | lacI binding site | ggaattgtgagcggataacaatttcacaca | |
| SEQ ID NO: | BBa_K1585117 | Anderson Promoter with | ... | 78 |
| 211 | | lacI binding site | ggaattgtgagcggataacaatttcacaca | |
| SEQ ID NO: | BBa_K1585118 | Anderson Promoter with | ... | 78 |
| 212 | | lacI binding site | ggaattgtgagcggataacaatttcacaca | |
| SEQ ID NO: | BBa_K1585119 | Anderson Promoter with | ... | 78 |
| 213 | | lacI binding site | ggaattgtgagcggataacaatttcacaca | |
| SEQ ID NO: | BBa_K1824896 | J23100 + RBS | ... | 88 |
| 214 | | | gattaaagaggagaaatactagagtactag | |
| SEQ ID NO: | BBa_K256002 | J23101:GFP | ... | 918 |
| 215 | | | caccttcgggtgggcctttctgcgtttata | |
| SEQ ID NO: | BBa_K256018 | J23119:IFP | ... | 1167 |
| 216 | | | caccttcgggtgggcctttctgcgtttata | |
| SEQ ID NO: | BBa_K256020 | J23119:HO1 | ... | 949 |
| 217 | | | caccttcgggtgggcctttctgcgtttata | |
| SEQ ID NO: | BBa_K256033 | Infrared signal reporter | ... | 2124 |
| 218 | | (J23119:IFP:J23119:HO1) | caccttcgggtgggcctttctgcgtttata | |
| SEQ ID NO: | BBa_K292000 | Double terminator + | ... | 138 |
| 219 | | constitutive promoter | ggctagctcagtcctaggtacagtgctagc | |
| SEQ ID NO: | BBa_K292001 | Double terminator + | ... | 161 |
| 220 | | Constitutive promoter + | tgctagctactagagattaaagaggagaaa | |
| | | Strong RBS | | |
| SEQ ID NO: | BBa_K418000 | IPTG inducible Lac | ... | 1416 |
| 221 | | promoter cassette | ttgtgagcggataacaagatactgagcaca | |
| SEQ ID NO: | BBa_K418002 | IPTG inducible Lac | ... | 1414 |
| 222 | | promoter cassette | ttgtgagcggataacaagatactgagcaca | |
| SEQ ID NO: | BBa_K418003 | IPTG inducible Lac | ... | 1416 |
| 223 | | promoter cassette | ttgtgagcggataacaagatactgagcaca | |
| SEQ ID NO: | BBa_K823004 | Anderson promoter | ... | 35 |
| 224 | | J23100 | ggctagctcagtcctaggtacagtgctagc | |
| SEQ ID NO: | BBa_K823005 | Anderson promoter | ... | 35 |
| 225 | | J23101 | agctagctcagtcctaggtattatgctagc | |
| SEQ ID NO: | BBa_K823006 | Anderson promoter | ... | 35 |
| 226 | | J23102 | agctagctcagtcctaggtactgtgctagc | |
| SEQ ID NO: | BBa_K823007 | Anderson promoter | ... | 35 |
| 227 | | J23103 | agctagctcagtcctagggattatgctagc | |
| SEQ ID NO: | BBa_K823008 | Anderson promoter | ... | 35 |
| 228 | | J23106 | ggctagctcagtcctaggtatagtgctagc | |
| SEQ ID NO: | BBa_K823010 | Anderson promoter | ... | 35 |
| 229 | | J23113 | ggctagctcagtcctagggattatgctagc | |
| SEQ ID NO: | BBa_K823011 | Anderson promoter | ... | 35 |
| 230 | | J23114 | ggctagctcagtcctaggtacaatgctagc | |
| SEQ ID NO: | BBa_K823013 | Anderson promoter | ... | 35 |
| 231 | | J23117 | agctagctcagtcctagggattgtgctagc | |
| SEQ ID NO: | BBa_K823014 | Anderson promoter | ... | 35 |
| 232 | | J23118 | ggctagctcagtcctaggtattgtgctagc | |
| SEQ ID NO: | BBa_M13101 | M13K07 gene I promoter | ...cctgtttttatgttattctctctgtaaagg | 47 |
| 233 | | | | |
| SEQ ID NO: | BBa_M13102 | M13K07 gene II promoter | ...aaatatttgcttatacaatcttcctgtttt | 48 |
| 234 | | | | |
| SEQ ID NO: | BBa_M13103 | M13K07 gene III | ... | 48 |
| 235 | | promoter | gctgataaaccgatacaattaaaggctcct | |
| SEQ ID NO: | BBa_M13104 | M13K07 gene IV | ... | 49 |
| 236 | | promoter | ctcttctcagcgtcttaatctaagctatcg | |
| SEQ ID NO: | BBa_M13105 | M13K07 gene V promoter | ... | 50 |
| 237 | | | atgagccagttcttaaaatcgcataaggta | |
| SEQ ID NO: | BBa_M13106 | M13K07 gene VI | ... | 49 |
| 238 | | promoter | ctattgattgtgacaaaataaacttattcc | |
| SEQ ID NO: | BBa_M13108 | M13K07 gene VIII | ... | 47 |
| 239 | | promoter | gtttcgcgcttggtataatcgctgggggtc | |
| SEQ ID NO: | BBa_M13110 | M13110 | ... | 48 |
| 240 | | | ctttgcttctgactataatagtcagggtaa | |
| SEQ ID NO: | BBa_M31519 | Modified promoter | ... | 60 |
| 241 | | sequence of g3. | aaaccgatacaattaaaggctcctgctagc | |
| SEQ ID NO: | BBa_R1074 | Constitutive Promoter I | ... | 74 |
| 242 | | | caccacactgatagtgctagtgtagatcac | |
| SEQ ID NO: | BBa_R1075 | Constitutive Promoter II | ... | 49 |
| 243 | | | gccggaataactccctataatgcgccacca | |
| SEQ ID NO: | BBa_S03331 | --Specify Parts List-- | ttgacaagcttttcctcagctccgtaaact | |
| 244 | | | | |

In some embodiments, the gene sequence(s) encoding a propionate catabolism enzyme is operably linked to a E. coli σS promoters. Exemplary E. coli σS promoters are listed in Table 9.

TABLE 9
Constitutive E. coli σ^{s} promoters

| SEQ | Name | Description | Promoter Sequence | Length |
|---|---|---|---|---|
| SEQ ID NO: | BBa_J45992 | Full-length stationary | ...ggtttcaaaattgtgatctatatttaacaa | 199 |
| 245 | | phase osmY promoter | | |
| SEQ ID NO: | BBa_J45993 | Minimal stationary | ...ggtttcaaaattgtgatctatatttaacaa | 57 |
| 246 | | phase osmY promoter | | |

In some embodiments, the gene sequence(s) encoding a propionate catabolism enzyme is operably linked to a E. coli σ³² promoters. Exemplary E. coli σ³² promoters are listed in Table 10.

TABLE 10
Constitutive E. coli σ³² promoters

| SEQ | Name | Description | Promoter Sequence | Length |
|---|---|---|---|---|
| SEQ ID NO: 247 | BBa_J45504 | htpG Heat Shock | ...tctattccaataaagaaatcttcctgcgtg | 405 |
| | | Promoter | | |
| SEQ ID NO: 248 | BBa_K1895002 | dnaK Promoter | ... | 182 |
| | | | gaccgaatatatagtggaaacgtttagatg | |
| SEQ ID NO: 249 | BBa_K1895003 | htpG Promoter | ...ccacatcctgtttttaaccttaaaatggca | 287 |

In some embodiments, the gene sequence(s) encoding a propionate catabolism enzyme is operably linked to a B. subtilis σ^{A} promoters. Exemplary B. subtilis σ^{A} promoters are listed in Table 11.

TABLE 11
Constitutive B. subtilisσ^{A} promoters

| SEQ | Name | Description | Promoter Sequence | Length |
|---|---|---|---|---|
| SEQ ID NO: 250 | BBa_K143012 | Promoter veg a | ... | 97 |
| | | constitutive promoter | aaaaatgggctcgtgttgtacaataaatgt | |
| | | for B. subtilis | | |
| SEQ ID NO: 251 | BBa_K143013 | Promoter 43 a | ... | 56 |
| | | constitutive promoter | aaaaaaagcgcgcgattatgtaaaatataa | |
| | | for B. subtilis | | |
| SEQ ID NO: 252 | BBa_K780003 | Strong constitutive | ... | 36 |
| | | promoter for Bacillus | aattgcagtaggcatgacaaaatggactca | |
| | | subtilis | | |
| SEQ ID NO: 253 | BBa_K823000 | P_{liaG} | ... | 121 |
| | | | caagcttttcctttataatagaatgaatga | |
| SEQ ID NO: 254 | BBa_K823002 | P_{lepA} | ...tctaagctagtgtattttgcgtttaatagt | 157 |
| SEQ ID NO: 255 | BBa_K823003 | P_{veg} | ... | 237 |
| | | | aatgggctcgtgttgtacaataaatgtagt | |

In some embodiments, the gene sequence(s) encoding a propionate catabolism enzyme is operably linked to a B. subtilis σB promoters. Exemplary B. subtilis σB promoters are listed in Table 12.

TABLE 12
Constitutive B. subtilis σ^{B} promoters

| SEQ | Name | Description | Promoter Sequence | Length |
|---|---|---|---|---|
| SEQ ID NO: 256 | BBa_K143010 | Promoter ctc for | ...atccttatcgttatgggtattgtttgtaat | 56 |
| | | B. subtilis | | |
| SEQ ID NO: 257 | BBa_K143011 | Promoter gsiB for | ... | 38 |
| | | B. subtilis | taaaagaattgtgagcgggaatacaacaac | |
| SEQ ID NO: 258 | BBa_K143013 | Promoter 43 a | ... | 56 |
| | | constitutive promoter | aaaaaaagcgcgcgattatgtaaaatataa | |
| | | for B. subtilis | | |

In some embodiments, the gene sequence(s) encoding a propionate catabolism enzyme is operably linked to promoters from Salmonella. Exemplary Salmonella promoters are listed in Table 13.

TABLE 13
Constitutive promoters from miscellaneous prokaryotes

| SEQ | Name | Description | Promoter Sequence | Length |
|---|---|---|---|---|
| SEQ ID NO: 259 | BBa_K112706 | Pspv2 | ... | 474 |
| | | from Salmonella | tacaaaataattcccctgcaaacattatca | |
| SEQ ID NO: 260 | BBa_K112707 | Pspv from Salmonella | ... | 1956 |
| | | | tacaaaataattcccctgcaaacattatcg | |

In some embodiments, the gene sequence(s) encoding a propionate catabolism enzyme is operably linked to promoters from bacteriophage T7. Exemplary promoters from bacteriophage T7 are listed in Table 14.

TABLE 14
Constitutive promoters from bacteriophage T7

| SEQ | Name | Description | Promoter Sequence | Length |
|---|---|---|---|---|
| SEQ ID NO: 261 | BBa_I712074 | T7 promoter (strong | ... | 46 |
| | | promoter from T7 | agggaatacaagctacttgttctttttgca | |
| | | bacteriophage) | | |
| SEQ ID NO: 262 | BBa_I719005 | T7 Promoter | taatacgactcactatagggaga | 23 |
| SEQ ID NO: 263 | BBa_J34814 | T7 Promoter | gaatttaatacgactcactatagggaga | 28 |
| SEQ ID NO: 264 | BBa_J64997 | T7 consensus −10 and | taatacgactcactatagg | 19 |
| | | rest | | |
| SEQ ID NO: 265 | BBa_K113010 | overlapping T7 | ... | 40 |
| | | promoter | gagtcgtattaatacgactcactatagggg | |
| SEQ ID NO: 266 | BBa_K113011 | more overlapping T7 | ... | 37 |
| | | promoter | agtgagtcgtactacgactcactatagggg | |
| SEQ ID NO: 267 | BBa_K113012 | weaken overlapping | ... | 40 |
| | | T7 promoter | gagtcgtattaatacgactctctatagggg | |
| SEQ ID NO: 268 | BBa_K1614000 | T7 promoter for | taatacgactcactatag | 18 |
| | | expression of | | |
| | | functional RNA | | |
| SEQ ID NO: 269 | BBa_R0085 | T7 Consensus | taatacgactcactatagggaga | 23 |
| | | Promoter Sequence | | |
| SEQ ID NO: 270 | BBa_R0180 | T7 RNAP promoter | ttatacgactcactatagggaga | 23 |
| SEQ ID NO: 271 | BBa_R0181 | T7 RNAP promoter | gaatacgactcactatagggaga | 23 |
| SEQ ID NO: 272 | BBa_R0182 | T7 RNAP promoter | taatacgtctcactatagggaga | 23 |
| SEQ ID NO: 273 | BBa_R0183 | T7 RNAP promoter | tcatacgactcactatagggaga | 23 |
| SEQ ID NO: 274 | BBa_Z0251 | T7 strong promoter | ... | 35 |
| | | | taatacgactcactatagggagaccacaac | |
| SEQ ID NO: 275 | BBa_Z0252 | T7 weak binding and | ... | 35 |
| | | processivity | taattgaactcactaaagggagaccacagc | |
| SEQ ID NO: 276 | BBa_Z0253 | T7 weak binding | ... | 35 |
| | | promoter | cgaagtaatacgactcactattagggaaga | |

In some embodiments, the gene sequence(s) encoding a propionate catabolism enzyme is operably linked to promoters bacteriophage SP6. Exemplary promoters from bacteriophage SP6 are listed in Table 15.

TABLE 15
Constitutive promoters from bacteriophage SP6

| SEQ | Name | Description | Promoter Sequence | Length |
|---|---|---|---|---|
| SEQ ID NO: | BBa_J64998 | consensus −10 and rest from SP6 | atttaggtgacactataga | 19 |
| 277 | | | | |

In some embodiments, the gene sequence(s) encoding a propionate catabolism enzyme is operably linked to promoters from yeast. Exemplary promoters from yeast are listed in Table 16.

TABLE 16
Constitutive promoters from yeast

| SEQ | Name | Description | Promoter Sequence | Length |
|---|---|---|---|---|
| SEQ ID NO: 278 | BBa_I766555 | pCyc (Medium) | ... | 244 |
| | | Promoter | acaaacacaaatacacacactaaattaata | |
| SEQ ID NO: 279 | BBa_I766556 | pAdh (Strong) Promoter | ... | 1501 |
| | | | ccaagcatacaatcaactatctcatataca | |
| SEQ ID NO: 280 | BBa_I766557 | pSte5 (Weak) Promoter | ... | 601 |
| | | | gatacaggatacagcggaaacaacttttaa | |
| SEQ ID NO: 281 | BBa_J63005 | yeast ADH1 promoter | ... | 1445 |
| | | | tttcaagctataccaagcatacaatcaact | |
| SEQ ID NO: 282 | BBa_K105027 | cyc100 minimal | ... cctttgcagcataaattactatacttctat | 103 |
| | | promoter | | |
| SEQ ID NO: 283 | BBa_K105028 | cyc70 minimal | ... cctttgcagcataaattactatacttctat | 103 |
| | | promoter | | |
| SEQ ID NO: 284 | BBa_K105029 | cyc43 minimal | ... cctttgcagcataaattactatacttctat | 103 |
| | | promoter | | |
| SEQ ID NO: 285 | BBa_K105030 | cyc28 minimal | ... cctttgcagcataaattactatacttctat | 103 |
| | | promoter | | |
| SEQ ID NO: 286 | BBa_K105031 | cyc16 minimal | ... cctttgcagcataaattactatacttctat | 103 |
| | | promoter | | |
| SEQ ID NO: 287 | BBa_K122000 | pPGK1 | ... ttatctactttttacaacaaatataaaaca | 1497 |
| SEQ ID NO: 288 | BBa_K124000 | pCYC Yeast Promoter | ... | 288 |
| | | | acaaacacaaatacacacactaaattaata | |
| SEQ ID NO: 289 | BBa_K124002 | Yeast GPD (TDH3) | ... | 681 |
| | | Promoter | gtttcgaataaacacacataaacaaacaaa | |
| SEQ ID NO: 290 | BBa_K319005 | yeast mid-length ADH1 | ... | 720 |
| | | promoter | ccaagcatacaatcaactatctcatataca | |
| SEQ ID NO: 291 | BBa_M31201 | Yeast CLB1 promoter | ... | 500 |
| | | region, G2/M cell cycle | accatcaaaggaagctttaatcttctcata | |
| | | specific | | |

In some embodiments, the gene sequence(s) encoding a propionate catabolism enzyme is operably linked to promoters from eukaryotes. Exemplary promoters from eukaryotes are listed in Table 17.

TABLE 17
Constitutive promoters from miscellaneous eukaryotes

| SEQ | Name | Description | Promoter Sequence | Length |
|---|---|---|---|---|
| SEQ ID NO: 292 | BBa_I712004 | CMV promoter | ... agaacccactgcttactggcttatcgaaat | 654 |
| SEQ ID NO: 293 | BBa_K076017 | Ubc Promoter | ... ggccgtttttggcttttttgttagacgaag | 1219 |

Other exemplary promoters are listed in Table 18.

TABLE 18
Other Constitutive Promoters

| SEQ | Name | Sequence | Description |
|---|---|---|---|
| SEQ ID | Plpp | ataagtgccttcccatcaaaaaaatatt | The Plpp promoter is a natural promoter |
| NO: 294 | | ctcaacataaaaaactttgtgtaatactt | taken from the Nissle genome. In situ it |
| | | gtaacgcta | is used to drive production of lpp, which |
| | | | is known to be the most abundant protein |
| | | | in the cell. Also, in some previous |
| | | | RNAseq experiments I was able to |
| | | | confirm that the lpp mRNA is one of the |
| | | | most abundant mRNA in Nissle during |
| | | | exponential growth. |
| SEQ ID | PapFAB46 | AAAAAGAGTATTGACT | See, e.g., Kosuri, S., Goodman, D. B. & |
| NO: 295 | | TCGCATCTTTTTGTACC | Cambray, G. Composability of |
| | | TATAATAGATTCATTGC | regulatory sequences controlling |
| | | TA | transcription and translation in |
| | | | Escherichia coli. in 1-20 (2013). |
| | | | doi: 10.1073/pnas. |
| SEQ ID | PJ23101 + | ggaaaatttttttaaaaaaaaaactttac | UP element helps recruit RNA |
| NO: 296 | UP | agctagctcagtcctaggtattatgcta | polymerase |
| | element | gc | (ggaaaatttttttaaaaaaaaaac) (SEQ ID NO: |
| | | | 314) |
| SEQ ID | PJ23107 + | ggaaaatttttttaaaaaaaaaactttac | UP element helps recruit RNA |
| NO: 297 | UP | ggctagctcagccctaggtattatgct | polymerase |
| | element | agc | (ggaaaatttttttaaaaaaaaaac) (SEQ ID NO: |
| | | | 314) |
| SEQ ID | PSYN23119 | ggaaaatttttttaaaaaaaaaacTT | UP element at 5′ end; consensus −10 |
| NO: 298 | | GACAGCTAGCTCAGTC | region is TATAAT; the consensus −35 is |
| | | CTTGGTATAATGCTAG | TTGACA; the extended −10 region is |
| | | CACGAA | generally TGNTATAAT |
| | | | (TGGTATAAT in this sequence) |

In some embodiments, the constitutive promoter is at least about 80%, at least about 85%, at least about 90%, at least about 95%, or at least about 99% homologous to the sequence of SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, and/or SEQ ID NO: 298.

### Induction of Payloads During Strain Culture

In some embodiments, it is desirable to pre-induce activity of one or more propionate catabolism enzyme(s) and/or propionate and/or methylmalonate importers (transporters) and/or succinate exporters prior to administration. Such propionate catabolism enzyme gene(s) and/or other protein(s) of interest can be an effector intended for secretion or can be an enzyme which catalyzes a metabolic reaction to produce an effector. In other embodiments, the protein of interest is an enzyme which catabolizes a harmful metabolite. In such situations, the strains are pre-loaded with active payload or protein of interest. In such instances, the genetically engineered bacteria of the invention express one or more propionate catabolism enzyme(s) and/or other protein(s) of interest, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of different cell culture conditions, including anaerobic or low oxygen or oxygenated conditions, in the presence of inducers, nutrients, at defined temperatures, and the like.

Culture conditions are selected to achieve optimal activity and viability of the cells, while maintaining a high cell density (high biomass) yield. A number of different cell culture conditions and operating parameters are monitored and adjusted to achieve optimal activity, high yield and high viability, including oxygen levels (e.g., low oxygen, microaerobic, aerobic), temperature of the medium, and nutrients and/or different growth media, chemical and/or nutritional inducers and other components provided in the medium.

In some embodiments, the one or more propionate catabolism enzyme(s) are directly or indirectly induced, while the strains are grown up for in vivo administration. Without wishing to be bound by theory, pre-induction may boost in vivo activity. In contrast, if a strain is pre-induced and preloaded, the strains are already fully active, allowing for greater activity more quickly as the bacteria reach the region of the intestine in which they are active, e.g., the gut. Ergo, no transit time is “wasted”, in which the strain is not optimally active. As the bacteria continue to move through the intestine, in vivo induction occurs under environmental conditions of the gut (e.g., low oxygen, or in the presence of gut metabolites).

In one embodiment, expression of one or more payload(s), is induced during cell growth, cell expansion, fermentation, recovery, purification, formulation, and/or manufacture. In one embodiment, induction of one or more promoters, each driving expression of one or more proteins of interest, occurs during cell growth, cell expansion, fermentation, recovery, purification, formulation, and/or manufacture. In one embodiment, expression of one or more payload(s) is driven from the same promoter. In one embodiment, expression of one or more payload(s) is driven from two or more copies of the same promoter. In one embodiment, expression of two or more payload(s) is driven from two or more copies of the same promoter and the two or more payloads are the same. In one embodiment, expression of two or more payload(s) is driven from the two or more copies of the same promoter and the two or more payload(s) are different. In one embodiment, expression of two or more payload(s) is driven from two or more copies of different promoter(s). In one embodiment, expression of one or more payload(s) is driven from two or more different promoter(s) and the two or more payload(s) are the same. In one embodiment, expression of two or more payload(s) is driven from two or more different promoter(s) and the two or more payload(s) are different. In one embodiment, expression of two or more of the same or different payload(s) is driven from the two or more copies of the same or different promoters. Payloads are expressed either from plasmid(s), the bacterial chromosome, or both.

In some embodiments, the strains are administered without any pre-induction protocols during strain growth prior to in vivo administration.

Anaerobic Induction

In some embodiments, cells are induced under strictly anaerobic or low oxygen conditions in culture. In such instances, cells are grown (e.g., for 1.5 to 3 hours) until they have reached a certain OD, e.g., ODs within the range of 0.1 to 10, indicating a certain density e.g., ranging from 1×10̂8 to 1×10̂11, and exponential growth and are then switched to strictly anaerobic or low oxygen conditions for approximately 3 to 5 hours. In some embodiments, strains are induced under strictly anaerobic or low oxygen conditions, e.g. to induce FNR promoter activity and drive expression of one or more payload(s) and/or Phe transporters under the control of one or more FNR promoters.

In one embodiment, expression of one or more one or more propionate catabolism enzyme(s) and/or propionate and/or methylmalonate importers (transporters) and/or succinate exporters, is under the control of one or more FNR promoter(s) and is induced during cell growth, cell expansion, fermentation, recovery, purification, formulation, and/or manufacture under strictly anaerobic or low oxygen conditions. In one embodiment, expression of several different propionate catabolism enzyme(s) and/or other protein(s) of interest is under the control of one or more FNR promoter(s) and is induced during cell growth, cell expansion, fermentation, recovery, purification, formulation, and/or manufacture under strictly anaerobic or low oxygen conditions.

Without wishing to be bound by theory, strains that comprise one or more propionate catabolism enzyme gene(s) and/or other polypeptide(s) of interest under the control of an FNR promoter, may allow expression of payload(s) from these promoters in vitro, under strictly anaerobic or low oxygen culture conditions, and in vivo, under the low oxygen conditions found in the gut.

In some embodiments, promoters inducible by arabinose, IPTG, rhamnose, tetracycline, and/or other chemical and/or nutritional inducers can be induced under strictly anaerobic or low oxygen conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more gene of interest sequence(s) under the control of one or more FNR promoter(s) and one or more same or different gene of interest sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) and/or under the control of one or more FNR promoter(s), and one or more same or different payload gene sequence(s) under the control of a one or more constitutive promoter(s) described herein. In some embodiments, strains may comprise one or more payload gene sequence(s) under the control of an FNR promoter and one or more same or different payload gene sequence(s) under the control of a one or more thermoregulated promoter(s) described herein.

In one embodiment, expression of one or more one or more propionate catabolism enzyme(s) and/or propionate and/or methylmalonate importers (transporters) and/or succinate exporters is under the control of one or more promoter(s) regulated by chemical and/or nutritional inducers and is induced during cell growth, cell expansion, fermentation, recovery, purification, formulation, and/or manufacture under strictly anaerobic and/or low oxygen conditions. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g., under strictly anaerobic and/or low oxygen conditions. In one embodiment, expression of two or more payload(s) is driven from two or more copies of the same promoter, e.g., under strictly anaerobic and/or low oxygen conditions. In one embodiment, expression of two or more payload(s) under strictly anaerobic and/or low oxygen conditions is driven from two or more copies of the same promoter and the payloads are the same. In one embodiment, expression of two or more payload(s) under strictly anaerobic and/or low oxygen conditions is driven from two or more copies of the same promoter and the payloads are different. In one embodiment, expression of two or more payload(s) under strictly anaerobic and/or low oxygen conditions is driven from two or more different promoter(s). In one embodiment, expression of two or more payload(s) under strictly anaerobic and/or low oxygen conditions is driven from two or more different promoter(s) and the payload(s) are the same. In one embodiment, expression of one or more payload(s) under strictly anaerobic and/or low oxygen conditions is driven from two or more different promoter(s), and the payload(s) are different. In one embodiment, expression of one or more of the same or different payload(s), under strictly anaerobic and/or low oxygen conditions, is driven from the one or more same or different promoters. Payloads are expressed either from plasmid(s), the bacterial chromosome, or both.

In one embodiment, strains may comprise a combination of gene sequence(s), some of which are under control of a first inducible promoter and others which are under control of a second inducible promoter, both induced by chemical and/or nutritional inducers, under strictly anaerobic or low oxygen conditions. In some embodiments, the strains comprise gene sequence(s) under the control of a. third inducible promoter, e.g., strictly anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains comprise payload under the control of one or more constitutive promoter(s) active under low oxygen conditions. In some embodiments, the bacteria strains comprise one or more payload under the control of one or more constitutive promoter(s) active and one or more inducible promoter(s), e.g., FNR and/or chemically, nutritionally and/or metabolite inducible and/or thermo regulated, under low oxygen conditions.

### Aerobic Induction

In some embodiments, it is desirable to prepare, pre-load and pre-induce the strains under aerobic conditions. This allows more efficient growth and viability, and, in some cases, reduces the build-up of toxic metabolites. In such instances, cells are grown (e.g., for 1.5 to 3 hours) until they have reached a certain OD, e.g., ODs within the range of 0.1 to 10, indicating a certain density e.g., ranging from 1×10̂8 to 1×10̂11, and exponential growth and are then induced through the addition of the inducer or through other means, such as shift to a permissive temperature, for approximately 3 to 5 hours.

In some embodiments, promoters inducible by one or more chemical and/or nutritional inducer(s) and or metabolite(s), e.g., by arabinose, IPTG, rhamnose, tetracycline, and/or other chemical and/or nutritional inducers described herein or known in the art can be induced under aerobic conditions in the presence of the chemical and/or nutritional and/or metabolite inducer during cell growth, cell expansion, fermentation, recovery, purification, formulation, and/or manufacture. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under aerobic conditions.

In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In some embodiments, promoters regulated by temperature are induced during cell growth, cell expansion, fermentation, recovery, purification, formulation, and/or manufacture. In one embodiment, expression of one or more payload(s) is driven directly or indirectly by one or more thermoregulated promoter(s) and is induced during cell growth, cell expansion, fermentation, recovery, purification, formulation, and/or manufacture under aerobic conditions.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g., under aerobic conditions. In one embodiment, expression of two or more payload(s) is driven from two or more copies of the same promoter, e.g., under aerobic conditions. In one embodiment, expression of two or more payload(s) under aerobic conditions is driven from two or more copies of the same promoter and the payloads are the same. In one embodiment, expression of two or more payload(s) under aerobic conditions is driven from two or more copies of the same promoter and the payloads are different. In one embodiment, expression of two or more payload(s) under aerobic conditions is driven from two or more different promoter(s). In one embodiment, expression of two or more payload(s) under aerobic conditions is driven from two or more different promoter(s) and the payload(s) are the same. In one embodiment, expression of one or more payload(s) under aerobic conditions is driven from two or more different promoter(s), and the payload(s) are different. In one embodiment, expression of one or more of the same or different payload(s), under aerobic conditions, is driven from the one or more same or different promoters. Payloads are expressed either from plasmid(s), the bacterial chromosome, or both.

In one embodiment, strains may comprise a combination of gene sequence(s) encoding one or more one or more propionate catabolism enzyme(s) and/or propionate and/or methylmalonate importers (transporters) and/or succinate exporters, some of which are under control of a first inducible promoter and others which are under control of a second inducible promoter, both induced under aerobic conditions. In some embodiments, a strain comprises three or more different promoters which are induced under aerobic culture conditions.

In one embodiment, strains may comprise a combination of gene sequence(s), some of which are under control of a first inducible promoter and others which are under control of a second inducible promoter, both induced by chemical and/or nutritional inducers. In one embodiment, strains may comprise a combination of gene sequence(s), some of which are under control of a first inducible promoter, e.g. a chemically inducible promoter, and others which are under control of a second inducible promoter, e.g. a temperature sensitive promoter under aerobic culture conditions. In some embodiments two or more chemically induced promoter gene sequence(s) are combined with a thermoregulated construct described herein. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) and/or Phe transporter gene sequence(s) and/or transcriptional regulator gene sequence(s) under the control of an FNR promoter and one or more payload gene sequence(s) and/or Phe transporter gene sequence(s) and/or transcriptional regulator gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload and or Phe transporter sequence(s) under the control of one or more constitutive promoter(s) active under aerobic conditions.

In some embodiments, genetically engineered strains comprise gene sequence(s) which are induced under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s), which are arabinose inducible under aerobic culture conditions. In some embodiments, these strains do not further comprise FNR inducible gene sequence(s) for in vivo activation in the gut.

In some embodiments, genetically engineered strains comprise gene sequence(s), which are IPTG inducible under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s) which are arabinose inducible under aerobic culture conditions. In some embodiments, such a strain further comprises sequence(s) which are IPTG inducible under aerobic culture conditions. In some embodiments, these strains further comprise FNR inducible gene payload and/or Phe transporter sequence(s) for in vivo activation in the gut. In some embodiments, these strains do not further comprise FNR inducible gene sequence(s) for in vivo activation in the gut.

As evident from the above non-limiting examples, genetically engineered strains comprise inducible gene sequence(s) which can be induced numerous combinations. For example, rhamnose or tetracycline can be used as an inducer with the appropriate promoters in addition or in lieu of arabinose and/or IPTG or with thermoregulation. Additionally, such bacterial strains can also be induced with the chemical and/or nutritional inducers under anaerobic conditions.

### Microaerobic Induction

In some embodiments, viability, growth, and activity are optimized by pre-inducing the bacterial strain under microaerobic conditions. In some embodiments, microaerobic conditions are best suited to “strike a balance” between optimal growth, activity and viability conditions and optimal conditions for induction; in particular, if the expression of the one or more payload(s) and/or Phe transporter(s) are driven by anaerobic and/or low oxygen promoter, e.g., a FNR promoter. In such instances, cells are grown (e.g., for 1.5 to 3 hours) until they have reached a certain OD, e.g., ODs within the range of 0.1 to 10, indicating a certain density e.g., ranging from 1×10̂8 to 1×10̂11, and exponential growth and are then induced through the addition of the inducer or through other means, such as shift to at a permissive temperature, for approximately 3 to 5 hours.

In one embodiment, expression of one or more payload(s) is under the control of one or more FNR promoter(s) and is induced during cell growth, cell expansion, fermentation, recovery, purification, formulation, and/or manufacture under microaerobic conditions.

Without wishing to be bound by theory, strains that comprise one or more payload(s), e.g., one or more propionate catabolism enzyme(s) and/or other polypeptides of interest, under the control of an FNR promoter, may allow expression of payload(s) from these promoters in vitro, under microaerobic culture conditions, and in vivo, under the low oxygen conditions found in the gut.

In some embodiments, promoters inducible by arabinose, IPTG, rhamnose, tetracycline, and/or other chemical and/or nutritional inducers can be induced under microaerobic conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) under the control of a one or more constitutive promoter(s) described herein. In some embodiments, strains may comprise one or more payload gene sequence(s) under the control of an FNR promoter and one or more payload gene sequence(s) under the control of a one or more thermoregulated promoter(s) described herein.

In one embodiment, expression of one or more payload(s) is under the control of one or more promoter(s) regulated by chemical and/or nutritional inducers and is induced during cell growth, cell expansion, fermentation, recovery, purification, formulation, and/or manufacture under microaerobic conditions.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g., under microaerobic conditions. In one embodiment, expression of two or more payload(s) is driven from two or more copies of the same promoter, e.g., under microaerobic conditions. In one embodiment, expression of two or more payload(s) under microaerobic conditions is driven from two or more copies of the same promoter and the payloads are the same. In one embodiment, expression of two or more payload(s) under microaerobic conditions is driven from two or more copies of the same promoter and the payloads are different. In one embodiment, expression of two or more payload(s) under microaerobic conditions is driven from two or more different promoter(s). In one embodiment, expression of two or more payload(s) under microaerobic conditions is driven from two or more different promoter(s) and the payload(s) are the same. In one embodiment, expression of one or more payload(s) under microaerobic conditions is driven from two or more different promoter(s), and the payload(s) are different. In one embodiment, expression of one or more of the same or different payload(s), under microaerobic conditions, is driven from the one or more same or different promoters. Payloads are expressed either from plasmid(s), the bacterial chromosome, or both.

In one embodiment, strains may comprise a combination of gene sequence(s), some of which are under control of a first inducible promoter and others which are under control of a second inducible promoter, both induced by chemical and/or nutritional inducers, under microaerobic conditions. In one embodiment, strains may comprise a combination of gene sequence(s), some of which are under control of a first inducible promoter and others which are under control of a second inducible promoter, both induced by chemical and/or nutritional inducers. In some embodiments, the strains comprise gene sequence(s) under the control of a third inducible promoter, e.g., an anaerobic/low oxygen promoter or microaerobic promoter, e.g., FNR promoter. In one embodiment, strains may comprise a combination of gene sequence(s), some of which are under control of a first inducible promoter, e.g., a chemically induced promoter or a low oxygen or microaerobic promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload under the control of one or more constitutive promoter(s) active under low oxygen conditions.

Induction of Strains Using Phasing, Pulsing and/or Cycling

In some embodiments, cycling, phasing, or pulsing techniques are employed during cell growth, expansion, recovery, purification, fermentation, and/or manufacture to efficiently induce and grow the strains prior to in vivo administration. This method is used to “strike a balance” between optimal growth, activity, cell health, and viability conditions and optimal conditions for induction; in particular, if growth, cell health or viability are negatively affected under inducing conditions. In such instances, cells are grown (e.g., for 1.5 to 3 hours) in a first phase or cycle until they have reached a certain OD, e.g., ODs within the range of 0.1 to 10, indicating a certain density e.g., ranging from 1×10̂8 to 1×10̂11, and are then induced through the addition of the inducer or through other means, such as shift to a permissive temperature (if a promoter is thermoregulated), or change in oxygen levels (e.g., reduction of oxygen level in the case of induction of an FNR promoter driven construct) for approximately 3 to 5 hours. In a second phase or cycle, conditions are brought back to the original conditions which support optimal growth, cell health and viability. Alternatively, if a chemical and/or nutritional inducer is used, then the culture can be spiked with a second dose of the inducer in the second phase or cycle.

In some embodiments, two cycles of optimal conditions and inducing conditions are employed (i.e., growth, induction, recovery and growth, induction). In some embodiments, three cycles of optimal conditions and inducing conditions are employed. In some embodiments, four or more cycles of optimal conditions and inducing conditions are employed. In a non-liming example, such cycling and/or phasing is used for induction under anaerobic and/or low oxygen conditions (e.g., induction of FNR promoters). In one embodiment, cells are grown to the optimal density and then induced under anaerobic and/or low oxygen conditions. Before growth and/or viability are negatively impacted due to stressful induction conditions, cells are returned to oxygenated conditions to recover, after which they are then returned to inducing anaerobic and/or low oxygen conditions for a second time. In some embodiments, these cycles are repeated as needed.

In some embodiments, growing cultures are spiked once with the chemical and/or nutritional inducer. In some embodiments, growing cultures are spiked twice with the chemical and/or nutritional inducer. In some embodiments, growing cultures are spiked three or more times with the chemical and/or nutritional inducer. In a non-limiting example, cells are first grown under optimal growth conditions up to a certain density, e.g., for 1.5 to 3 hour) to reached an of 0.1 to 10, until the cells are at a density ranging from 1×10̂8 to 1×10̂11. Then the chemical inducer, e.g., arabinose or IPTG, is added to the culture. After 3 to 5 hours, an additional dose of the inducer is added to re-initiate the induction. Spiking can be repeated as needed.

In some embodiments, phasing or cycling changes in temperature in the culture. In another embodiment, adjustment of temperature may be used to improve the activity of a payload. For example, lowering the temperature during culture may improve the proper folding of the payload. In such instances, cells are first grown at a temperature optimal for growth (e.g., 37 C). In some embodiments, the cells are then induced, e.g., by a chemical inducer, to express the payload. Concurrently or after a set amount of induction time, the temperature in the media is lowered, e.g., between 25 and 35 C, to allow improved folding of the expressed payload.

In some embodiments, payload(s) are under the control of different inducible promoters, for example two different chemical inducers. In other embodiments, the payload is induced under low oxygen conditions or microaerobic conditions and a second payload is induced by a chemical inducer.

In one embodiment, expression of one or more payload(s) is under the control of one or more FNR promoter(s) and is induced during cell growth, cell expansion, fermentation, recovery, purification, formulation, and/or manufacture by using phasing or cycling or pulsing or spiking techniques.

In some embodiments, promoters inducible by arabinose, IPTG, rhamnose, tetracycline, and/or other chemical and/or nutritional inducers can be induced through the employment of phasing or cycling or pulsing or spiking techniques in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) and/or Phe transporter gene sequence(s) and/or transcriptional regulator gene sequence(s) under the control of a one or more constitutive promoter(s) described herein and are induced through the employment of phasing or cycling or pulsing or spiking techniques. In some embodiments, strains may comprise one or more payload gene sequence(s) under the control of an FNR promoter and one or more payload gene sequence(s) under the control of a one or more thermoregulated promoter(s) described herein, and are induced through the employment of phasing or cycling or pulsing or spiking techniques.

Any of the strains described herein can be grown through the employment of phasing or cycling or pulsing or spiking techniques. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under anaerobic and/or low oxygen conditions.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g., through the employment of phasing or cycling or pulsing or spiking techniques. In one embodiment, expression of two or more payload(s) is driven from two or more copies of the same promoter, through the employment of phasing or cycling or pulsing or spiking techniques. In one embodiment, expression of two or more payload(s), regulated through the employment of phasing or cycling or pulsing or spiking techniques, is driven from two or more copies of the same promoter and the payloads are the same. In one embodiment, expression of two or more payload(s), regulated through the employment of phasing or cycling or pulsing or spiking techniques is driven from two or more copies of the same promoter and the payloads are different. In one embodiment, expression of two or more payload(s), regulated through the employment of phasing or cycling or pulsing or spiking techniques is driven from two or more different promoter(s). In one embodiment, expression of two or more payload(s), regulated through the employment of phasing or cycling or pulsing or spiking techniques, is driven from two or more different promoter(s) and the payload(s) are the same. In one embodiment, expression of one or more payload(s), regulated through the employment of phasing or cycling or pulsing or spiking techniques, is driven from two or more different promoter(s), and the payload(s) are different. In one embodiment, expression of one or more of the same or different payload(s), regulated through the employment of phasing or cycling or pulsing or spiking techniques, is driven from the one or more same or different promoters. Payloads are expressed either from plasmid(s), the bacterial chromosome, or both.

In one embodiment, strains may comprise a combination of gene sequence(s), some of which are under control of a first inducible promoter and others which are under control of a second inducible promoter, both induced by chemical and/or nutritional inducers, through the employment of phasing or cycling or pulsing or spiking techniques. In one embodiment, strains may comprise a combination of gene sequence(s), some of which are under control of a first inducible promoter and others which are under control of a second inducible promoter, both induced by chemical and/or nutritional inducers through the employment of phasing or cycling or pulsing or spiking techniques. In some embodiments, the strains comprise gene sequence(s) under the control of a third inducible promoter, e.g., an anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequence(s) under the control of one or more constitutive promoter(s) active under low oxygen conditions. Any of the strains described in these embodiments may be induced through the employment of phasing or cycling or pulsing or spiking techniques.

### Aerobic Induction of the FNR Promoter

FNRS24Y is a mutated form of FNR which is more resistant to inactivation by oxygen, and therefore can activate FNR promoters under aerobic conditions (see e.g., Jervis A J The O2 sensitivity of the transcription factor FNR is controlled by Ser24 modulating the kinetics of [4Fe-4S] to [2Fe-2S] conversion, Proc Natl Acad Sci USA. 2009 Mar. 24; 106(12):4659-64, the contents of which is herein incorporated by reference in its entirety). In some embodiments, oxygen bypass system shown and described in FIG. 39A is used. In this oxygen bypass system, FNRS24Y is induced by addition of arabinose and then drives the expression a propionate catabolizing enzyme (POI1) and/or a importer/transporter and/or exporter (POI2) by binding and activating the FNR promoter under aerobic conditions. Thus, strains can be grown, produced or manufactured efficiently under aerobic conditions, while being effectively pre-induced and pre-loaded, as the system takes advantage of the strong FNR promoter resulting in of high levels of expression of POI1 and PO2. This system does not interfere with or compromise in vivo activation, since the mutated FNRS24Y is no longer expressed in the absence of arabinose, and wild type FNR then binds to the FNR promoter and drives expression of POI1 and POI2.

In some embodiments, FNRS24Y is expressed during aerobic culture growth and induces a gene of interest. In other embodiments described herein, a second payload expression can also be induced aerobically, e.g., by arabinose. In a non-limiting example, a protein of interest and FNRS24Y can in some embodiments be induced simultaneously, e.g., from an arabinose inducible promoter. In some embodiments, FNRS24Y and the protein of interest are transcribed as a bicistronic message whose expression is driven by an arabinose promoter. In some embodiments, FNRS24Y is knocked into the arabinose operon, allowing expression to be driven from the endogenous Para promoter.

In some embodiments, a Lad promoter and IPTG induction are used in this system (in lieu of Para and arabinose induction). In some embodiments, a rhamnose inducible promoter is used in this system. In some embodiments, a temperature sensitive promoter is used to drive expression of FNRS24Y.

Sequences useful for expression from inducible promoters are listed in Table 56.

Propionate Catabolism Enzymes and Propionate Catabolism Genes and Gene Cassettes

As used herein, the term “propionate catabolism gene,” “propionate catabolism gene cassette,” “propionate catabolism cassette”, or “propionate catabolism operon” refers to a gene or set of genes capable of catabolizing propionate, and/or a metabolite thereof, and/or methylmalonic acid, an/or a metabolite thereof, in a biosynthetic pathway.

As used herein, the term “propionate catabolism enzyme” or “propionate catabolic or catabolism enzyme” or “propionate metabolic enzyme” refers to any enzyme that is capable of metabolizing propionate and/or a metabolite thereof. The term “propionate catabolism enzyme” or “propionate catabolic or catabolism enzyme” or “propionate metabolic enzyme” refers to any enzyme that is capable of metabolizing propionate and/or methylmalonic acid and/or a metabolite thereof. For example, the term “propionate catabolism enzyme” or “propionate catabolic or catabolism enzyme” or “propionate metabolic enzyme” refers to any enzyme that is capable of metabolizing propionate, propionyl-CoA, methylmalonic acid, and/or methylmalonyl CoA. For example, the term “propionate catabolism enzyme” or “propionate catabolic or catabolism enzyme” or “propionate metabolic enzyme” refers to any enzyme that is capable of reducing accumulated propionate and/or methylmalonic acid and/or propionyl CoA and/or methylmalonyl CoA or that can lessen, ameliorate, or prevent one or more propionate and/or methylmalonic acid diseases or disease symptoms. Examples of propionate and/or methylmalonic acid metabolic enzymes include, but are not limited to, propionyl CoA carboxylase (PCC), methylmalonyl CoA mutase (MUT), propionyl-CoA synthetase (PrpE), 2-methylisocitrate lyase (PrpB), 2-methylcitrate synthase (prpC), 2-methylcitrate dehydratase (PrpD), propionyl-CoA carboxylase (pccB), Acetyl-/propionyl-coenzyme A carboxylase (accA1), Methylmalonyl-CoA epimerase (mmcE), methylmalonyl-CoA mutase (mutA, and mutB), Acetoacetyl-CoA reductase (phaB), Polyhydroxyalkanoic acid (PHA) synthases, e.g., encoded by phaC, and 3-ketothiolase (phaA), pct, and malonyl-coenzyme A (malonyl-CoA) synthetase (matB).

Functional deficiencies in these proteins result in the accumulation of propionate and/or methylmalonic acid or one or more of their metabolites in cells and tissues. Propionate catabolism enzymes of the present disclosure include both wild-type or modified propionate catabolism enzymes and can be produced using recombinant and synthetic methods or purified from nature sources. Propionate catabolism enzymes include full-length polypeptides and functional fragments thereof, as well as homologs and variants thereof. Propionate catabolism enzymes include polypeptides that have been modified from the wild-type sequence, including, for example, polypeptides having one or more amino acid deletions, insertions, and/or substitutions and may include, for example, fusion polypeptides and polypeptides having additional sequence, e.g., regulatory peptide sequence, linker peptide sequence, and other peptide sequence.

As used herein, the term “propionate catabolism enzyme” refers to an enzyme involved in the catabolism of propionate or propionyl CoA and or methylmalonic acid or methylmalonyl CoA to a non-toxic molecule, such as its corresponding methylmalonyl CoA molecule, corresponding succinyl CoA molecule, succinate, or polyhydroxyalkanoates; or the catabolism of methylmalonyl CoA to non-toxic molecule, such as its corresponding succinyl CoA molecule. Enzymes involved in the catabolism of propionate are well known to those of skill in the art.

In humans, the major pathway for metabolizing propionyl-CoA involves the enzyme propionyl CoA carboxylase (PCC), which converts propionyl CoA to methylmalonyl CoA, and the methylmalonyl CoA mutase (MUT) enzyme then converts methylmalonyl CoA into succinyl CoA (see, e.g., FIG. 5). Enzyme deficiencies or mutations which lead to the toxic accumulation of propionyl CoA or methylmalonyl CoA result in the development of disorders associated with propionate catabolism, such as PA and MMA, and severe nutritional deficiencies of Vitamin B₁₂ can also result in MMA (Higginbottom et al., M. Engl. J. Med., 299(7):317-323, 1978). Other minor pathways are present in humans, but these pathways are insufficient to compensate for the absence of or mutations in the major pathway for propionyl CoA metabolism (see, e.g., FIG. 5). Thus, in some embodiments, the engineered bacterium comprises gene sequence(s) encoding one or more copies of propionyl CoA carboxylase (PCC). In some embodiments, the engineered bacterium comprises gene sequence(s) encoding one or more copies of propionyl CoA carboxylase (PCC) and one or more copies of methylmalonyl CoA mutase (MUT).

For propionic acid to be consumed by any of the pathways or circuits of the present disclosure, it must first be activated to propionyl-CoA. This activation can be catalyzed by either propionyl-CoA synthetase (PrpE) or propionate CoA transferase (Pct). Thus, in some embodiments, the engineered bacterium comprises gene sequence(s) encoding one or more copies of propionyl-CoA synthetase (PrpE). In some embodiments, the engineered bacterium comprises gene sequence(s) encoding one or more copies of propionate CoA transferase (Pct). In some embodiments, the engineered bacterium comprises gene sequence(s) encoding one or more copies of propionyl-CoA synthetase (PrpE) and one or more copies of propionyl CoA carboxylase (PCC). In some embodiments, the engineered bacterium comprises gene sequence(s) encoding one or more copies of propionyl-CoA synthetase (PrpE), one or more copies of propionyl CoA carboxylase (PCC) and one or more copies of methylmalonyl CoA mutase (MUT). In some embodiments, the engineered bacterium comprises gene sequence(s) encoding one or more copies of propionate CoA transferase (Pct) and one or more copies of propionyl CoA carboxylase (PCC). In some embodiments, the engineered bacterium comprises gene sequence(s) encoding one or more copies of propionate CoA transferase (Pct), one or more copies of propionyl CoA carboxylase (PCC) and one or more copies of methylmalonyl CoA mutase (MUT).

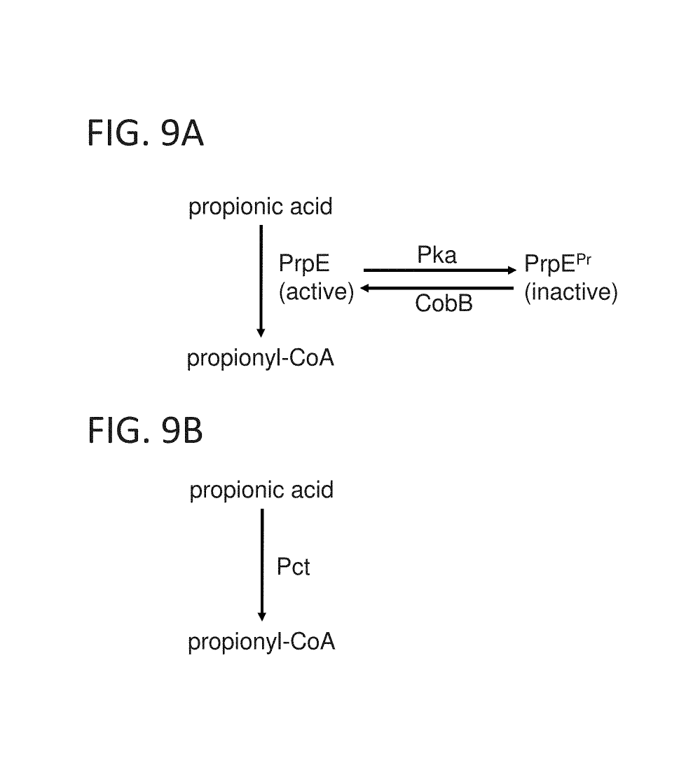

PrpE converts propionate and free CoA to propionyl-CoA in an irreversible, ATP-dependent manner, releasing AMP and PPi (pyrophosphate). PrpE can be inactivated by postranslational modification of the active site lysine, e.g., as shown in FIG. 9A. Protein lysine acetyltransferase (Pka) in E. coli carries out the propionylation of PrpE. The enzyme CobB depropionylates PrpEPr making the inactivation reversible. However, the inactivation pathway can be eliminated entirely through the deletion of the pka gene. In any of the embodiments described herein and elsewhere in the specification, the genetically engineered bacteria comprise a deletion of pka (Δpka) to prevent the inactivation of PrpE. In some embodiments the deletion of pka results in greater activity of PrpE and downstream catabolic enzymes.

Pct converts propionate and acetyl-CoA to propionyl-CoA and acetate in a reversible reaction. In some embodiments, the genetically engineered bacteria comprise a gene encoding Pct for the generation of propionyl CoA from propionate, e.g., as shown in FIG. 9B. In some embodiments, the genetically engineered bacteria comprise Pct in combination with or as a component of one or more of PHA and/or MMCA and/or 2MC pathway cassette(s).

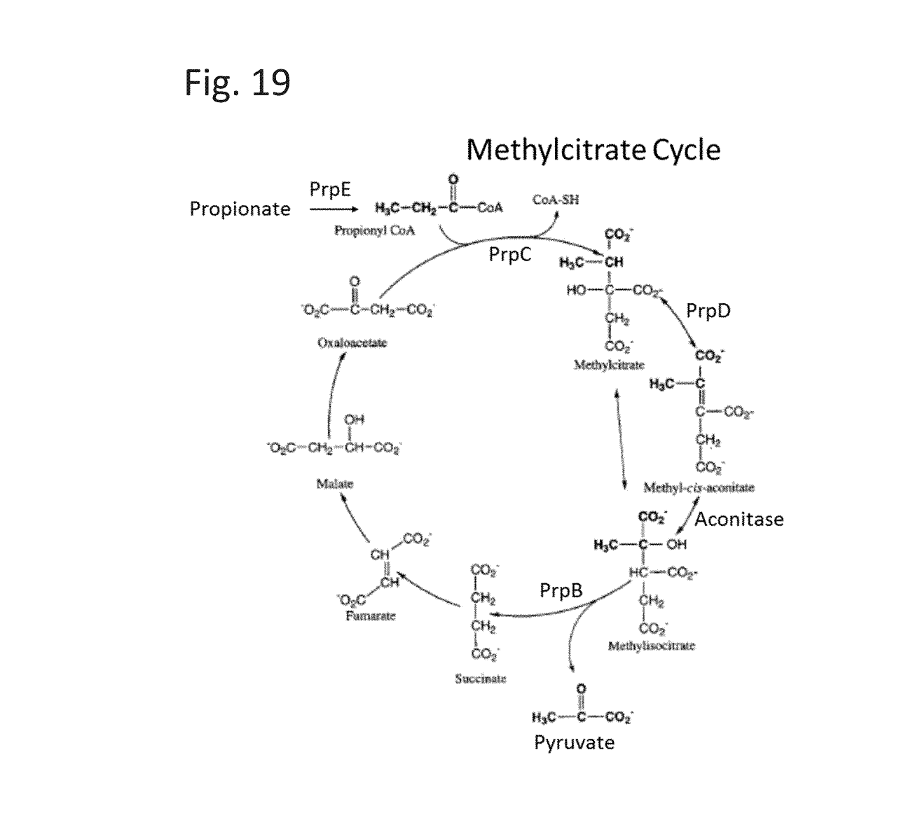

In bacteria, PrpB, PrpC, and PrpD are capable of converting propionyl CoA into succinate and pyruvate, and PrpB, PrpC, PrpD, and PrpE are capable of converting propionate into succinate and pyruvate. Specifically, PrpE, a propionate-CoA ligase, converts propionate to propionyl CoA. PrpC, a 2-methylcitrate synthetase, then converts propionyl CoA to 2-methylcitrate. PrpD, a 2-methylcitrate dehydrogenase, then converts 2-methylcitrate into 2-methyisocitrate, and PrpB, a 2-methylisocitrate lyase, converts 2-methyisocitrate into succinate and pyruvate (see FIG. 19). Thus, in some embodiments, the engineered bacterium comprises gene sequence(s) encoding one or more of the following: PrpB, PrpC, and PrpD. In some embodiments, the engineered bacterium comprises gene sequence(s) encoding one or more of the following: PrpB, PrpC, PrpD, and PrpE. In some embodiments, the engineered bacterium comprises two or more copies of a gene encoding any of the following: PrpB, PrpC, and PrpD, and combinations thereof. In some embodiments, the engineered bacterium comprises two or more copies of a gene encoding any of the following: PrpB, PrpC, PrpD, and PrpE, and combinations thereof.

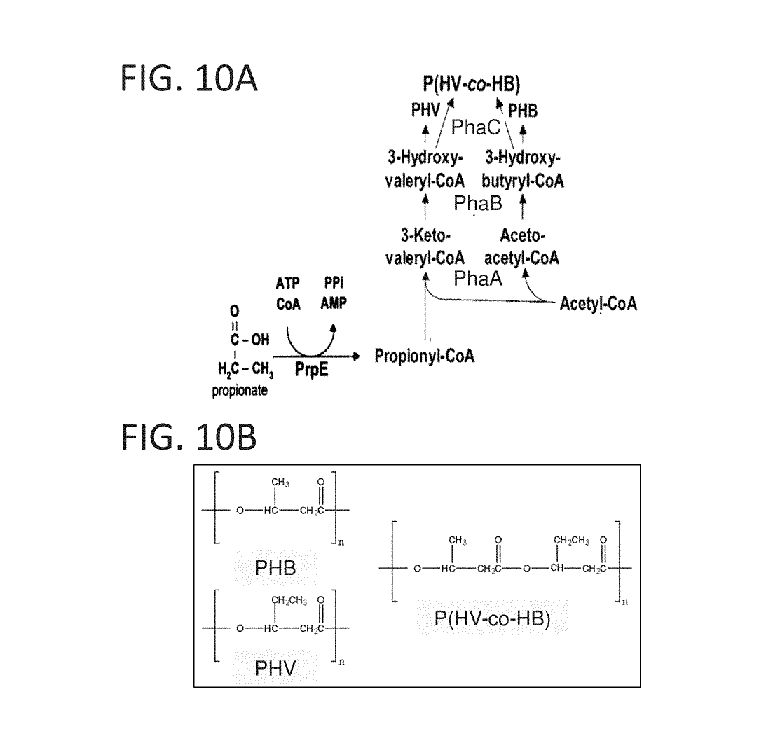

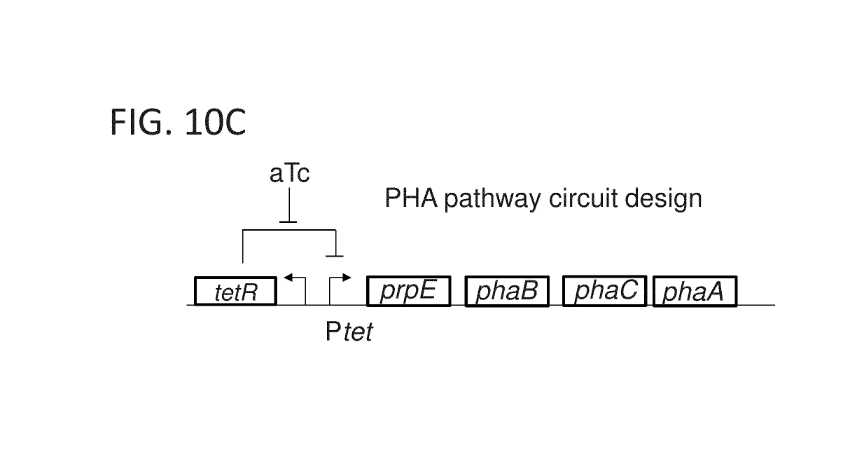

In another bacterial pathway, the polyhydroxyalkanoate pathway, propionate is converted to propionyl-CoA by PrpE. Propionyl-CoA is then converted to 3-keto-valeryl-CoA by PhaA, which is then converted to 3-hydroxy-valeryl-CoA by PhaB. Finally, PhaC converts 3-hydroxy-valeryl-CoA to PHV (see FIG. 10). Thus, in some embodiments, the engineered bacterium comprises gene sequence(s) encoding one or more of the following: PrpE, PhaA, and PhaB.

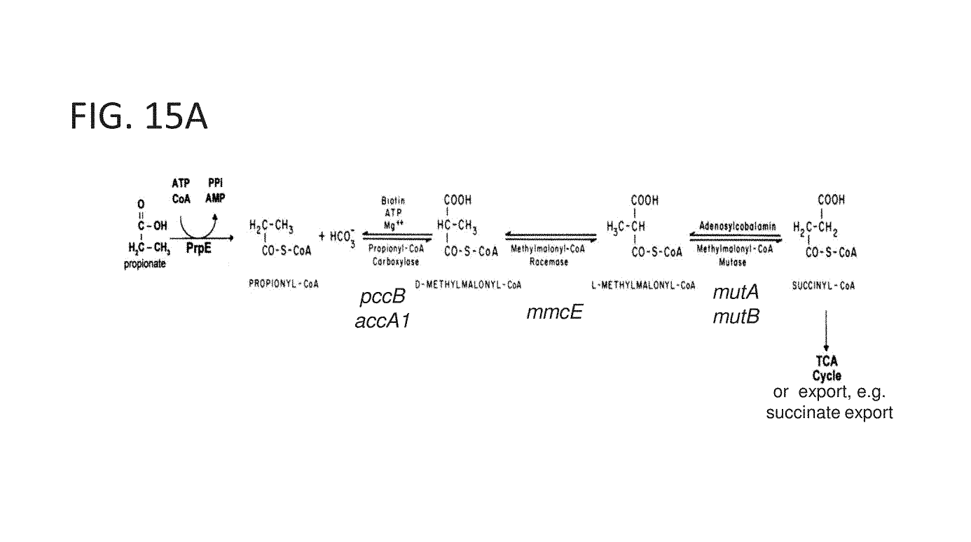

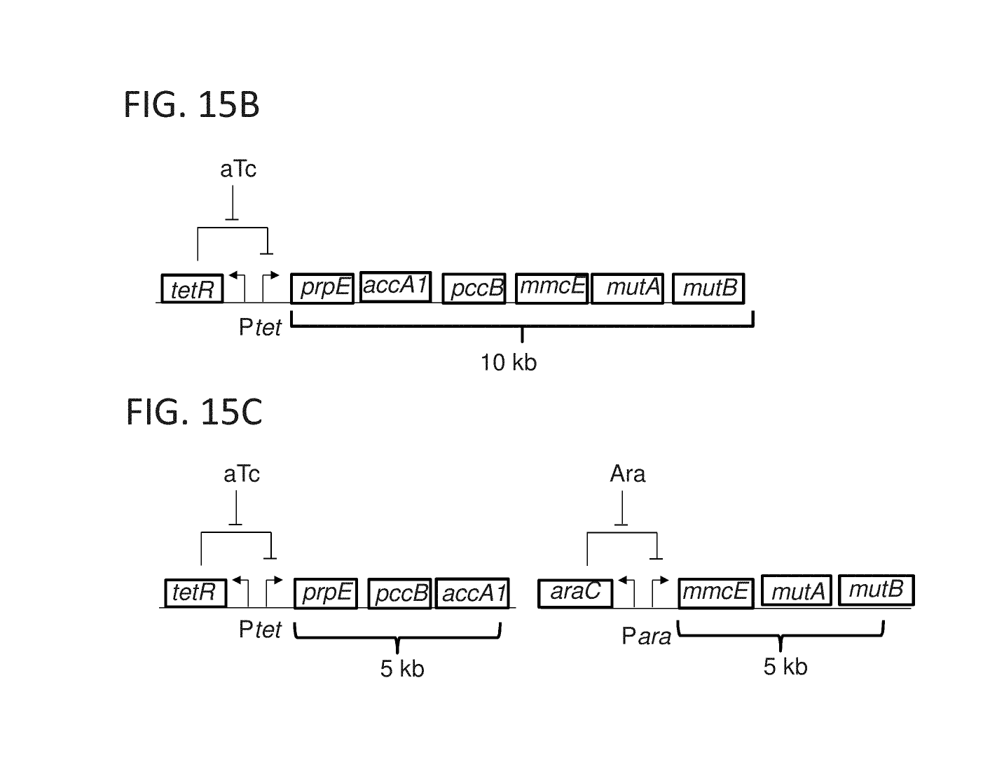

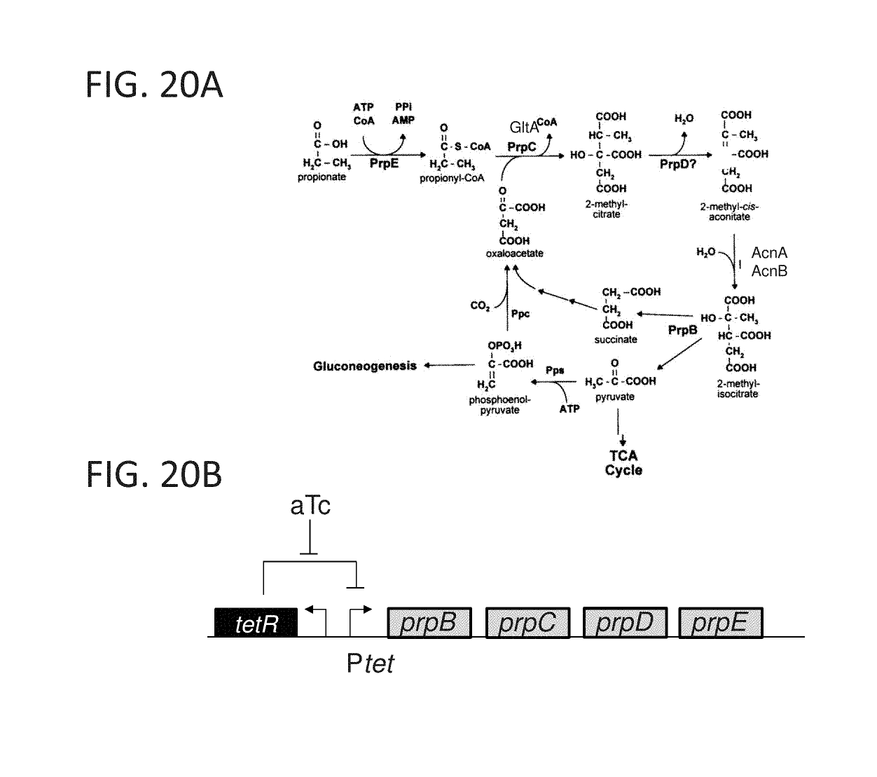

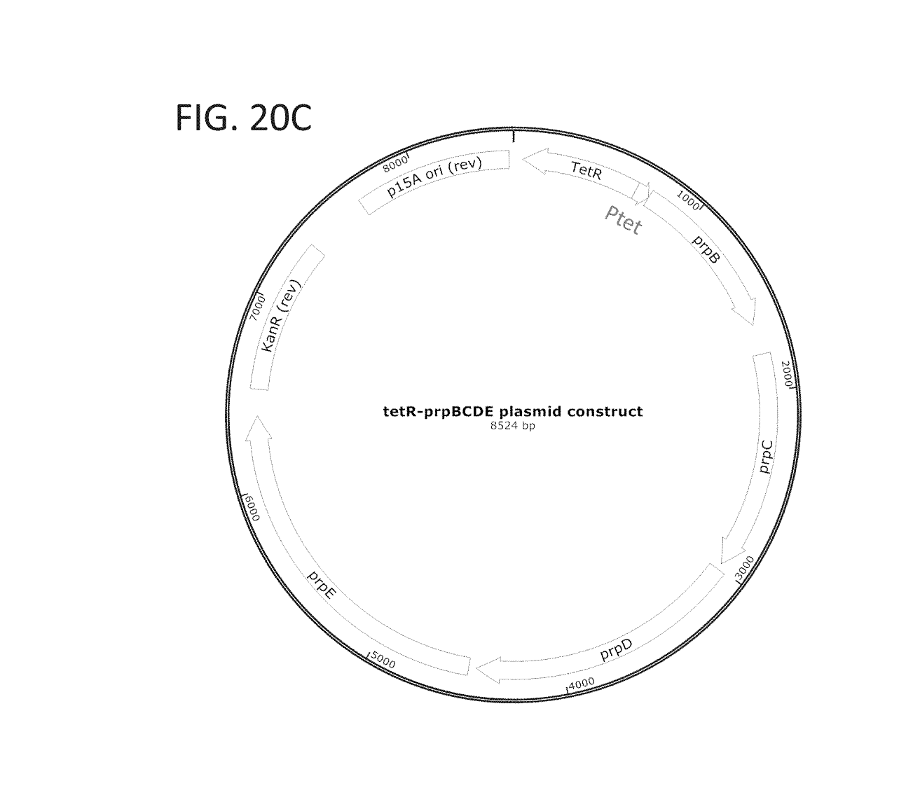

The disclosure encompasses the design of genetic circuits which mimic the functional activities of the human methylmalonyl-CoA pathway in order to catabolize propionate to treat diseases associated with propionate catabolism. For example, a circuit can be designed to express prpE, pccB, accA1, mmcE, mutA, and mutB (FIG. 15). In this circuit, PrpE converts propionate to propionyl-CoA, which is then converted to D-methylmalonyl-CoA by PccB and AccA1. D-methylmalonyl-CoA is then converted to L-methylmalonyl-CoA by MmcE, and MutA and MutB convert L-methylmalonyl CoA to succinyl-CoA. Alternatively, these genes can be split up into two circuits, i.e., prpE-accA1-pccB and mmcE-mutA-mutB, as indicated in FIG. 15. Thus, in some embodiments, the engineered bacterium comprises gene sequence(s) selected from: prpE, pccB, accA1, mmcE, mutA, and mutB. In some embodiments, the engineered bacterium comprises gene sequence(s) encoding one or more of the following: PrpE, PccB, AccA1, MmcE, MutA, and MutB. In another embodiment, the disclosure encompasses the design of genetic circuits which constitute the 2-methylcitrate cycle pathway in bacteria, such as the prpBCDE circuit (FIG. 20) or the polyhydroxyalkanoate pathway, such as the prpE, phaB, phaC, phaA genes (FIG. 10C) in order to catabolize propionate to treat diseases associated with propionate catabolism.

The disclosure encompasses the design of genetic circuits which comprise MatB. Malonyl-coenzyme A (malonyl-CoA) synthetase (MatB) belongs to the AMP-forming acyl-CoA synthetase protein family. These enzymes catalyze the conversion of organic acids to acyl-CoA thioesters via a ping-pong mechanism, in which ATP and the organic acid are first converted to acyl-AMP with the release of pyrophosphate, followed by coenzyme A binding, displacement of AMP, and release of the acyl-CoA product (see, e.g., Crosby et al., Structure-Guided Expansion of the Substrate Range of Methylmalonyl Coenzyme A Synthetase (MatB) of Rhodopseudomonas palustris; Appl. Environ. Microbiol. September 2012 vol. 78 no. 18 6619-6629, and references therein). MatB converts malonate to malonyl-CoA in two steps according to this mechanism via a malonyl-AMP intermediate, and similarly also converts methylmalonate to methylmalonyl-CoA.

A genetic circuit comprising MatB is useful in the treatment of methylmalonic acidemia, allowing accumulated methylmalonic acid to be converted into methylmalonyl CoA. Once converted to methylmalonyl CoA, catabolism can proceed along the MMCA pathway (e.g., through mmcE, mutA, and mutB). Alternatively, methylmalonyl CoA can be converted to propionyl CoA. This reaction may be catalyzed by the AccA1/PccB complex, which is encoded by a genetic circuit of the disclosure. The AccA1/pccB complex catalyzes the reversible conversion of propionyl CoA to methylmalonylCoA, as described herein. Once methylmalonyl CoA is converted to propionyl CoA, any of the propionate catabolism enzymes encoded by the genetic circuits described herein, e.g., PHA, MMCA, and/or 2MC circuits, are suitable for further catalysis, resulting in an inert product. Thus, in any of the embodiments described herein and elsewhere in the specification, the engineered bacterium may further comprise gene sequence(s) encoding MatB.

In some embodiments of the disclosure, one or more gene(s) or gene cassette(s) comprise MatB, e.g., MatB derived from Rhodopseudomonas palustris. In some embodiments of the disclosure, the genetically engineered bacteria comprise one or more gene(s) or gene cassette(s) comprising MatB, e.g., MatB derived from Rhodopseudomonas palustris. In a non-limiting example, genetically engineered bacteria comprising one or more gene(s) or gene cassettes comprising MatB are suitable for the treatment of methylmalonic acidemia or methylmalonic acidemia and propionic acidemia.

In some embodiments, the genetically engineered bacteria comprise one or more gene(s) or gene cassette(s) encoding MatB and one or more MMCA gene cassettes as described herein. In some embodiments, the genetically engineered bacteria comprise one or more gene(s) or gene cassette(s) encoding MatB and one or more MMCA gene(s) or MMCA gene cassette(s) as described herein. In some embodiments, MatB is driven by a separate promoter and is on a separate plasmid or chromosomal integration site. In some embodiments, MatB part of an operon comprising one or more gene(s) or gene cassette(s) encoding one or more propionate catabolism enzymes described herein.

In some embodiments, the genetically engineered bacteria encode one or more of MatB, mmcE, mutA, and mutB. In some embodiments, the genetically engineered bacteria encode MatB, mmcE, mutA, and mutB. In some embodiments, a genetic circuit encoded by the genetically engineered bacteria comprises MatB, mmcE, mutA, and mutB.

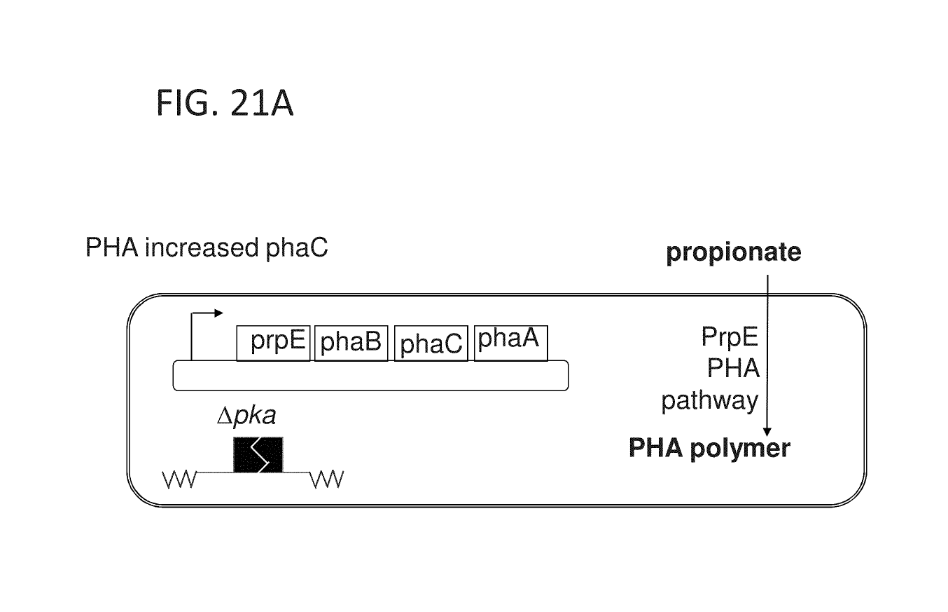

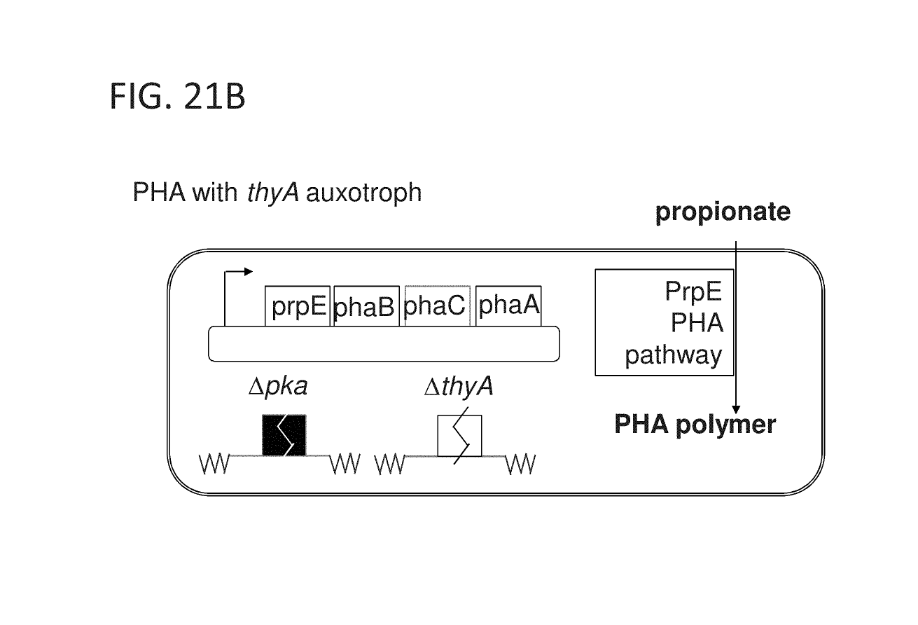

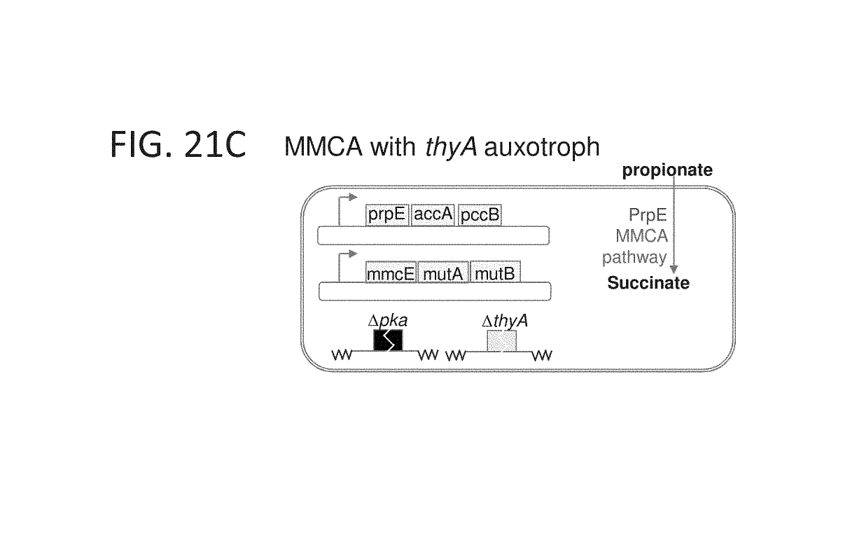

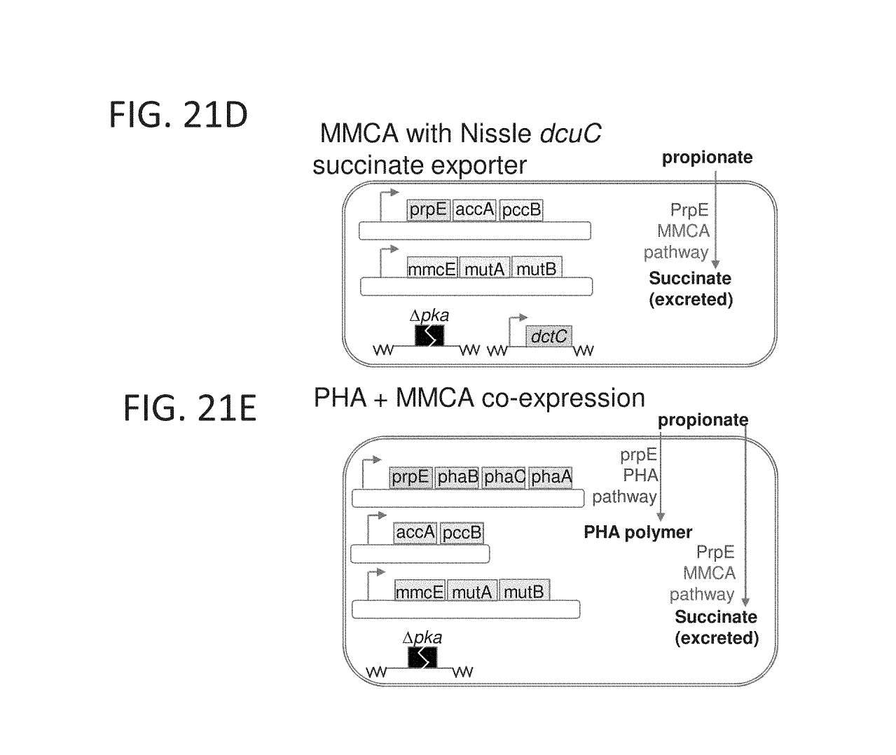

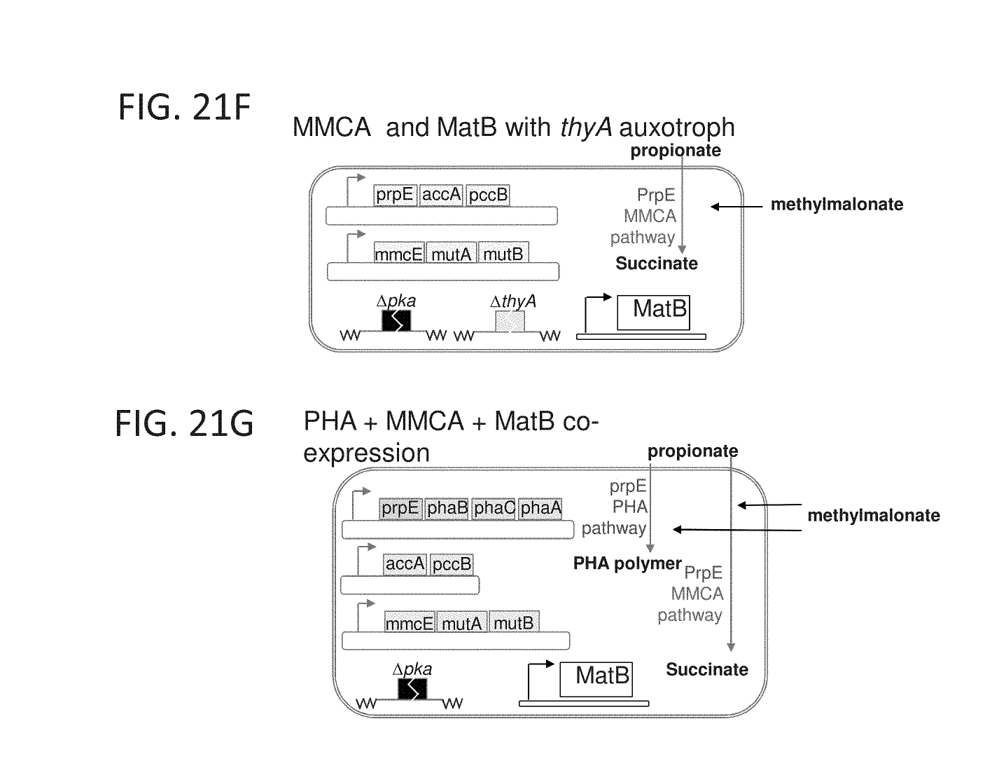

In some embodiments, the genetically engineered bacteria encode one or more of MatB, Acc1A, and PccB. In some embodiments, the genetically engineered bacteria encode MatB, Acc1A, and PccB. In some embodiments, a genetic circuit encoded by the genetically engineered bacteria comprises MatB, Acc1A, and PccB. In some embodiments, the genetically engineered bacteria encode MatB, Acc1A, and PccB, and mmcE, mutA and mutB. In some embodiments, the genetically engineered bacteria encode MatB, Acc1A, and PccB, and mmcE, mutA and mutB and further prpE. In some embodiments, the genetically engineered bacteria encode MatB, Acc1A, and PccB, and mmcE, mutA and mutB, and further encode a PHA and/or 2MC pathway circuit, and may or may not further comprise prpE. These genes may be organized in one or more gen cassettes, as described herein. Non-limiting examples of genetically engineered bacteria comprising one or more gene(s) or gene cassettes and comprising exemplary operons or gene cassette(s) are depicted in FIG. 21G and FIG. 21F. In other non-limiting examples, the one or more gene cassettes may be organized as follows; MatB-mmcE-mutA-mutB; MatB-Acc1A-PccB and mmcE-mutA-mutB, alone or in combination with PPHA and/or 2MC pathway cassettes; PrpE-MatB-Acc1A-PccB and mmcE-mutA-mutB, alone or in combination with PPHA and/or 2MC pathway cassettes.

In one embodiment, expression of the propionate catabolism gene cassette increases the rate of propionate, propionyl CoA, methylmalonate and/or methylmalonyl CoA catabolism in the cell. In one embodiment, expression of the propionate catabolism gene cassette decreases the level of propionate in the cell. In another embodiment, expression of the propionate catabolism gene cassette decreases the level of propionic acid in the cell. In one embodiment, expression of the propionate catabolism gene cassette decreases the level of propionyl CoA in the cell. In one embodiment, expression of the propionate catabolism gene cassette decreases the level of methylmalonyl CoA in the cell. In one embodiment, expression of the propionate catabolism gene cassette decreases the level of methylmalonic acid in the cell.

In another embodiment, expression of the propionate catabolism gene cassette increases the level of methylmalonyl CoA in the cell as compared to the level of its corresponding propionyl CoA in the cell. In another embodiment, expression of the propionate catabolism gene cassette increases the level of succinate in the cell as compared to the level of its corresponding methylmalonyl CoA in the cell. In one embodiment, expression of the propionate catabolism gene cassette decreases the level of the propionate, propionyl CoA, methylmalonate and/or methylmalonyl CoA as compared to the level of succinate or succinyl CoA in the cell. In one embodiment, expression of the propionate catabolism gene cassette increases the level of succinate or succinyl CoA in the cell as compared to the level of the propionate, propionyl CoA, methylmalonate and/or methylmalonyl CoA in the cell.

Enzymes involved in the catabolism of propionate may be expressed or modified in the bacteria in order to enhance catabolism of propionate. Specifically, when the heterologous propionate catabolism gene or gene cassette is expressed in the engineered bacterial cells, the bacterial cells convert more propionate and/or propionyl CoA into methylmalonyl CoA, or convert more methylmalonyl CoA into succinate or succinyl CoA when the gene or gene cassette is expressed than unmodified bacteria of the same bacterial subtype under the same conditions. Thus, the genetically engineered bacteria expressing a heterologous propionate catabolism gene or gene cassette can catabolize propionate, propionyl CoA, methylmalonate and/or methylmalonyl CoA to treat diseases associated with catabolism of propionate, such as Propionic Acidemia (PA) and Methylmalonic Acidemia (MMA).

In some embodiments, the expression of the propionate catabolism gene cassette decreases the levels of one or more propionic acidemia and/or methylmalonic acidemia biomarkers. In some embodiments, the propionate catabolism gene cassette expressed by the genetically engineered bacteria decreases the levels of one or more propionic acidemia and/or methylmalonic acidemia biomarkers. In one embodiment, expression of the propionate catabolism gene cassette decreases the propionylcarnitine to acetylcarnitine ratio in the blood and/or the urine, e.g., in a mammalian subject with elevated levels of propionate and/or methylmalonate. In one embodiment, expression of the propionate catabolism gene cassette decreases levels of 2-methylcitrate in the blood and/or in the urine, e.g., in a mammalian subject with elevated levels of propionate and/or methylmalonate. In one embodiment, expression of the propionate catabolism gene cassette decreases levels of propionylglycine in the blood and/or in the urine, e.g., in a mammalian subject with elevated levels of propionate and/or methylmalonate. In one embodiment, expression of the propionate catabolism gene cassette decreases levels of tiglyglycine in the blood and/or in the urine, e.g., in a mammalian subject with elevated levels of propionate and/or methylmalonate.

In one embodiment, the bacterial cell comprises at least one heterologous gene encoding at least one propionate catabolism enzyme. In one embodiment, the bacterial cell comprises at least one heterologous gene encoding a transporter of propionate and at least one heterologous gene encoding at least one propionate catabolism enzyme.

In one embodiment, the engineered bacterial cell comprises at least one heterologous gene or gene cassette encoding at least one propionate catabolism enzyme. In some embodiments, the disclosure provides a bacterial cell that comprises at least one heterologous gene or gene cassette encoding at least one propionate catabolism enzyme operably linked to a first promoter. In one embodiment, the bacterial cell comprises at least one gene or gene cassette encoding at least one propionate catabolism enzyme from a different organism, e.g., a different species of bacteria. In another embodiment, the bacterial cell comprises more than one copy of a native gene or gene cassette encoding one or more propionate catabolism enzyme(s). In yet another embodiment, the bacterial cell comprises at least one native gene or gene cassette encoding at least one native propionate catabolism enzyme, as well as at least one copy of at least one gene or gene cassette encoding one or more propionate catabolism enzyme(s) from a different organism, e.g., a different species of bacteria. In one embodiment, the bacterial cell comprises at least one, two, three, four, five, or six copies of a gene or gene cassette encoding one or more propionate catabolism enzyme(s). In one embodiment, the bacterial cell comprises multiple copies of a gene or gene cassette encoding one or more propionate catabolism enzyme(s). In one embodiment, a gene cassette may comprise one or more native and one or more non-native or heterologous genes.

Multiple distinct propionate catabolism enzymes are known in the art. In some embodiments, the propionate catabolism enzyme is encoded by at least one gene encoding at least one propionate catabolism enzyme derived from a bacterial species. In some embodiments, a propionate catabolism enzyme is encoded by one or more gene(s) or gene cassettes encoding a propionate catabolism enzyme derived from a non-bacterial species. In some embodiments, a propionate catabolism enzyme is encoded by a gene derived from a eukaryotic species, e.g., a yeast species or a plant species. In one embodiment, a propionate catabolism enzyme is encoded by a gene derived from a human. In one embodiment, the at least one gene encoding the at least one propionate catabolism enzyme is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Pseudomonas, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas aeruginosa, Psychrobacter anticus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae.

In some embodiments, the gene encoding prpE is derived from E. coli. In some embodiments, the gene encoding accA1 is derived from Streptopmyces coelicolor. In some embodiments, the gene encoding pccB is derived from E. coli. In some embodiments, the gene encoding mmcE is derived from Propionibacterium freudenreichii. In some embodiments, the gene encoding mutA is derived from Propionibacterium freudenreichii. In some embodiments, the gene encoding mutB is derived from Propionibacterium freudenreichii. In some embodiments, the gene encoding prpB is derived from E. coli. In some embodiments, the gene encoding prpC is derived from E. coli. In some embodiments, the gene encoding prpD is derived from E. coli. In some embodiments, the gene encoding phaB is derived from Acinetobacter sp RA3849. In some embodiments, the gene encoding phaC is derived from Acinetobacter sp RA3849. In some embodiments, the gene encoding phaA is derived from Acinetobacter sp RA3849.

In one embodiment, the at least one gene encoding the at least one propionate catabolism enzyme has been codon-optimized for use in the engineered bacterial cell. In one embodiment, the at least one gene or gene cassette encoding the one or more propionate catabolism enzyme(s) has been codon-optimized for use in Escherichia coli. When the at least one gene encoding the at least one propionate catabolism enzyme is expressed in the engineered bacterial cells, the bacterial cells catabolize more propionate or propionyl CoA than unmodified bacteria of the same bacterial subtype under the same conditions (e.g., culture or environmental conditions). Thus, the genetically engineered bacteria comprising at least one heterologous gene or gene cassette encoding one or more propionate catabolism enzyme(s) may be used to catabolize excess propionate, propionic acid, and/or propionyl CoA to treat a disease associated with the catabolism of propionate, such as Propionic Acidemia, Methylmalonic Acidemia, or a vitamin B₁₂ deficiency.

The present disclosure further comprises genes and gene cassettes encoding functional fragments of a propionate catabolism enzyme or functional variants of a propionate catabolism enzyme(s). As used herein, the term “functional fragment thereof” or “functional variant thereof” of a propionate catabolism enzyme relates to an element having qualitative biological activity in common with the wild-type propionate catabolism enzyme from which the fragment or variant was derived. For example, a functional fragment or a functional variant of a mutated propionate catabolism enzyme is one which retains essentially the same ability to catabolize propionyl CoA and/or methylmalonyl CoA as the propionate catabolism enzyme from which the functional fragment or functional variant was derived. For example, a polypeptide having propionate catabolism enzyme activity may be truncated at the N-terminus or C-terminus and the retention of propionate catabolism enzyme activity assessed using assays known to those of skill in the art, including the exemplary assays provided herein. In one embodiment, the engineered bacterial cell comprises a heterologous gene encoding a propionate catabolism enzyme functional variant. In another embodiment, the engineered bacterial cell comprises a heterologous gene or gene cassette encoding a propionate catabolism enzyme functional fragment.

Assays for testing the activity of a propionate catabolism enzyme, a propionate catabolism enzyme functional variant, or a propionate catabolism enzyme functional fragment are well known to one of ordinary skill in the art. For example, propionate catabolism can be assessed by expressing the protein, functional variant, or fragment thereof, in an engineered bacterial cell that lacks endogenous propionate catabolism enzyme activity. In another example, propionate can be supplemented in the media, and engineered bacterial strains can be compared with corresponding wild type strains with respect to propionate depletion from the media, as described herein. Propionate levels can be assessed using mass spectrometry or gas chromatography. For example, samples can be injected into a Perkin Elmer Autosystem XL Gas Chromatograph containing a Supelco packed column, and the analysis can be performed according to manufacturing instructions (see, for example, Supelco I (1998) Analyzing fatty acids by packed column gas chromatography, Bulletin 856B:2014). Alternatively, propionate levels can be determined using high-pressure liquid chromatography (HPLC). For example, a computer-controlled Waters HPLC system equipped with a model 600 quaternary solvent delivery system, and a model 996 photodiode array detector, and components of a sample can be resolved with an Aminex HPX-87H (300 by 7.8 mm) organic acid analysis column (Bio-Rad Laboratories) (see, for example, Palacios et al., 2003, J. Bacteriol., 185(9):2802-2810).

In mammals, levels of certain propionate byproducts or metabolites, e.g., propionylcarnitine/acetylcarnitine ratios, 2-methyl-citrate, propionylglycine, and/or tiglyglycine, can be measured in addition to propionate levels by mass spec as described herein.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The present disclosure encompasses genes encoding a propionate catabolism enzyme comprising amino acids in its sequence that are substantially the same as an amino acid sequence described herein Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Be, Asn, Gln, Phe, Cys, Pro, Trp, Tyr, Val).

In some embodiments, the gene(s) or gene cassette(s) encoding propionate catabolism enzyme(s) are mutagenized; mutants exhibiting increased activity are selected; and the mutagenized gene(s) or mutagenized gene cassettes) encoding the propionate catabolism enzyme(s) are isolated and inserted into the bacterial cell. In one embodiment, spontaneous mutants that arise that allow bacteria to grow on propionate as the sole carbon source can be screened for and selected. The gene(s) comprising the modifications described herein may be present on a plasmid or chromosome.

In one embodiment, the at least one gene encoding the at least one propionate catabolism enzyme is prpE. prpE encodes PrpE, a propionate-CoA ligase. Accordingly, in one embodiment, the prpE gene has at least about 80% identity with SEQ ID NO: 25. In another embodiment, the prpE gene has at least about 80% identity with SEQ ID NO: 73. Accordingly, in one embodiment, the prpE gene has at least about 90% identity with SEQ ID NO: 25. In another embodiment, the prpE gene has at least about 90% identity with SEQ ID NO: 73. Accordingly, in one embodiment, the prpE gene has at least about 95% identity with SEQ ID NO: 25. In another embodiment, the prpE gene has at least about 95% identity with SEQ ID NO: 73. Accordingly, in one embodiment, the prpE gene has at least about 85%, 86%, 87%, 88%, 89%, 90%, 91%, 92%, 93%, 94%, 95%, 96%, 97%, 98%, or 99% identity with SEQ ID NO: 25. In another embodiment, the prpE gene has at least about 85%, 86%, 87%, 88%, 89%, 90%, 91%, 92%, 93%, 94%, 95%, 96%, 97%, 98%, or 99% identity with SEQ ID NO: 73. In another embodiment, the prpE gene comprises the sequence of SEQ ID NO: 25. In another embodiment, the prpE gene comprises the sequence of SEQ ID NO: 73. In yet another embodiment the prpE gene consists of the sequence of SEQ ID NO: 25. In another embodiment, the prpE gene consists of the sequence of SEQ ID NO: 73.

In one embodiment, the at least one gene encoding the at least one propionate catabolism enzyme is prpC. prpC encodes PrpC, a 2-methylcitrate synthetase. Accordingly, in one embodiment, the prpC gene has at least about 80% identity with SEQ ID NO: 57. In another embodiment, the prpC gene has at least about 80% identity with SEQ ID NO:76. Accordingly, in one embodiment, the prpC gene has at least about 90% identity with SEQ ID NO: 57. In another embodiment, the prpC gene has at least about 90% identity with SEQ ID NO: 76. Accordingly, in one embodiment, the prpC gene has at least about 95% identity with SEQ ID NO: 57. In another embodiment, the prpC gene has at least about 95% identity with SEQ ID NO: 76. Accordingly, in one embodiment, the prpC gene has at least about 85%, 86%, 87%, 88%, 89%, 90%, 91%, 92%, 93%, 94%, 95%, 96%, 97%, 98%, or 99% identity with SEQ ID NO: 57. In another embodiment, the prpC gene has at least about 85%, 86%, 87%, 88%, 89%, 90%, 91%, 92%, 93%, 94%, 95%, 96%, 97%, 98%, or 99% identity with SEQ ID NO: 76. In another embodiment, the prpC gene comprises the sequence of SEQ ID NO: 57. In another embodiment, the prpC gene comprises the sequence of SEQ ID NO: 76. In yet another embodiment the prpC gene consists of the sequence of SEQ ID NO: 57. In another embodiment, the prpC gene consists of the sequence of SEQ ID NO: 76.

In one embodiment, the at least one gene encoding the at least one propionate catabolism enzyme is prpD. prpD encodes PrpD, a 2-methylcitrate dehydrogenase. Accordingly, in one embodiment, the prpD gene has at least about 80% identity with SEQ ID NO: 58. In another embodiment, the prpD gene has at least about 80% identity with SEQ ID NO: 79. Accordingly, in one embodiment, the prpD gene has at least about 90% identity with SEQ ID NO: 58. In another embodiment, the prpD gene has at least about 90% identity with SEQ ID NO: 79. Accordingly, in one embodiment, the prpD gene has at least about 95% identity with SEQ ID NO: 58. In another embodiment, the prpD gene has at least about 95% identity with SEQ ID NO: 79. Accordingly, in one embodiment, the prpD gene has at least about 85%, 86%, 87%, 88%, 89%, 90%, 91%, 92%, 93%, 94%, 95%, 96%, 97%, 98%, or 99% identity with SEQ ID NO: 58. In another embodiment, the prpD gene has at least about 85%, 86%, 87%, 88%, 89%, 90%, 91%, 92%, 93%, 94%, 95%, 96%, 97%, 98%, or 99% identity with SEQ ID NO: 79. In another embodiment, the prpD gene comprises the sequence of SEQ ID NO: 58. In another embodiment, the prpD gene comprises the sequence of SEQ ID NO: 79. In yet another embodiment the prpD gene consists of the sequence of SEQ ID NO: 58. In another embodiment, the prpD gene consists of the sequence of SEQ ID NO: 79.

In one embodiment, the at least one gene encoding the at least one propionate catabolism enzyme is prpB. prpB encodes PrpB, a 2-methylisocitrate lyase. Accordingly, in one embodiment, the prpB gene has at least about 80% identity with SEQ ID NO: 56. In another embodiment, the prpB gene has at least about 80% identity with SEQ ID NO: 82. Accordingly, in one embodiment, the prpB gene has at least about 90% identity with SEQ ID NO: 56. In another embodiment, the prpB gene has at least about 90% identity with SEQ ID NO: 82. Accordingly, in one embodiment, the prpB gene has at least about 95% identity with SEQ ID NO: 56. In another embodiment, the prpB gene has at least about 95% identity with SEQ ID NO: 82. Accordingly, in one embodiment, the prpB gene has at least about 85%, 86%, 87%, 88%, 89%, 90%, 91%, 92%, 93%, 94%, 95%, 96%, 97%, 98%, or 99% identity with SEQ ID NO: 56. In another embodiment, the prpB gene has at least about 85%, 86%, 87%, 88%, 89%, 90%, 91%, 92%, 93%, 94%, 95%, 96%, 97%, 98%, or 99% identity with SEQ ID NO: 82. In another embodiment, the prpB gene comprises the sequence of SEQ ID NO: 56. In another embodiment, the prpB gene comprises the sequence of SEQ ID NO: 82. In yet another embodiment the prpB gene consists of the sequence of SEQ ID NO: 56. In another embodiment, the prpB gene consists of the sequence of SEQ ID NO: 82.

In one embodiment, the at least one gene encoding the at least one propionate catabolism enzyme is phaB. phaB encodes PhaB, a acetoacetyl-CoA reductase. Accordingly, in one embodiment, the phaB gene has at least about 80% identity with SEQ ID NO: 26. In one embodiment, the phaB gene has at least about 90% identity with SEQ ID NO: 26. In another embodiment, the phaB gene has at least about 95% identity with SEQ ID NO: 26. Accordingly, in one embodiment, the phaB gene has at least about 85%, 86%, 87%, 88%, 89%, 90%, 91%, 92%, 93%, 94%, 95%, 96%, 97%, 98%, or 99% identity with SEQ ID NO: 26. In another embodiment, the phaB gene comprises SEQ ID NO: 26. In yet another embodiment the phaB gene consists of SEQ ID NO: 26.

In one embodiment, the at least one gene encoding the at least one propionate catabolism enzyme is phaC. phaC encodes PhaC, a polyhydroxyalkanoate synthase. Accordingly, in one embodiment, the phaC gene has at least about 80% identity SEQ ID NO: 27. In one embodiment, the phaC gene has at least about 90% identity with SEQ ID NO: 27. In another embodiment, the phaC gene has at least about 95% identity with SEQ ID NO: 27. Accordingly, in one embodiment, the phaC gene has at least about 85%, 86%, 87%, 88%, 89%, 90%, 91%, 92%, 93%, 94%, 95%, 96%, 97%, 98%, or 99% identity with SEQ ID NO: 27. In another embodiment, the phaC gene comprises SEQ ID NO: 27. In yet another embodiment the phaC gene consists of SEQ ID NO: 27.

In one embodiment, the at least one gene encoding the at least one propionate catabolism enzyme is phaA. phaA encodes PhaA, a beta-ketothiolase. Accordingly, in one embodiment, the phaA gene has at least about 80% identity with a sequence which encodes SEQ ID NO: 28. In one embodiment, the phaA gene has at least about 90% identity with a sequence which encodes SEQ ID NO: 28. In another embodiment, the phaA gene has at least about 95% identity with a sequence which encodes SEQ ID NO: 28. Accordingly, in one embodiment, the phaA gene has at least about 85%, 86%, 87%, 88%, 89%, 90%, 91%, 92%, 93%, 94%, 95%, 96%, 97%, 98%, or 99% identity with a sequence which encodes SEQ ID NO: 28. In another embodiment, the phaA gene comprises a sequence which encodes SEQ ID NO: 28. In yet another embodiment the phaA gene consists of a sequence which encodes SEQ ID NO: 28.

In one embodiment, the at least one gene encoding the at least one propionate catabolism enzyme is pccB. pccB encodes PccB, a propionyl CoA carboxylase. Accordingly, in one embodiment, the pccB gene has at least about 80% identity with SEQ ID NO: 39. In one embodiment, the pccB gene has at least about 90% identity with SEQ ID NO: 39. In one embodiment, the pccB gene has at least about 95% identity with SEQ ID NO: 39. In one embodiment, the pccB gene has at least about 85%, 86%, 87%, 88%, 89%, 90%, 91%, 92%, 93%, 94%, 95%, 96%, 97%, 98%, or 99% identity with SEQ ID NO: 39. In another embodiment, the pccB gene comprises the sequence of SEQ ID NO: 39. In yet another embodiment, the pccB gene consists of the sequence of SEQ ID NO: 39.

In one embodiment, the at least one gene encoding the at least one propionate catabolism enzyme is pccB. Accordingly, in one embodiment, the pccB gene has at least about 80% identity with SEQ ID NO: 96. In one embodiment, the pccB gene has at least about 90% identity with SEQ ID NO: 96. In one embodiment, the pccB gene has at least about 95% identity with SEQ ID NO: 96. In one embodiment, the pccB gene has at least about 85%, 86%, 87%, 88%, 89%, 90%, 91%, 92%, 93%, 94%, 95%, 96%, 97%, 98%, or 99% identity with SEQ ID NO: 96. In another embodiment, the pccB gene comprises the sequence of SEQ ID NO: 96. In yet another embodiment, the pccB gene consists of the sequence of SEQ ID NO: 96.

In one embodiment, the at least one gene encoding the at least one propionate catabolism enzyme is accA1. accA1 encodes AccA1, an acetyl CoA carboxylase. Accordingly, in one embodiment, the accA1 gene has at least about 80% identity with SEQ ID NO: 38. In one embodiment, the accA1 gene has at least about 90% identity with SEQ ID NO: 38. In one embodiment, the accA1 gene has at least about 95% identity with SEQ ID NO: 38. In one embodiment, the accA1 gene has at least about 85%, 86%, 87%, 88%, 89%, 90%, 91%, 92%, 93%, 94%, 95%, 96%, 97%, 98%, or 99% identity with SEQ ID NO: 38. In another embodiment, the accA1 gene comprises the sequence of SEQ ID NO: 38. In yet another embodiment, the accA1 gene consists of the sequence of SEQ ID NO: 38.

In one embodiment, the at least one gene encoding the at least one propionate catabolism enzyme is accA1. accA1 encodes AccA1, an acetyl CoA carboxylase. Accordingly, in one embodiment, the accA1 gene has at least about 80% identity with SEQ ID NO: 104. In one embodiment, the accA1 gene has at least about 90% identity with SEQ ID NO: 104. In one embodiment, the accA1 gene has at least about 95% identity with SEQ ID NO: 104. In one embodiment, the accA1 gene has at least about 85%, 86%, 87%, 88%, 89%, 90%, 91%, 92%, 93%, 94%, 95%, 96%, 97%, 98%, or 99% identity with SEQ ID NO: 104. In another embodiment, the accA1 gene comprises the sequence of SEQ ID NO: 104. In yet another embodiment, the accA1 gene consists of the sequence of SEQ ID NO: 104.

In one embodiment, the at least one gene encoding the at least one propionate catabolism enzyme is mmcE. mmcE encodes MmcE, a methylmalonyl-CoA mutase. Accordingly, in one embodiment, the mmcE gene has at least about 80% identity with SEQ ID NO: 32. In one embodiment, the mmcE gene has at least about 90% identity with SEQ ID NO: 32. In one embodiment, the mmcE gene has at least about 95% identity with SEQ ID NO: 32. In one embodiment, the mmcE gene has at least about 85%, 86%, 87%, 88%, 89%, 90%, 91%, 92%, 93%, 94%, 95%, 96%, 97%, 98%, or 99% identity with SEQ ID NO: 32. In another embodiment, the mmcE gene comprises the sequence of SEQ ID NO: 32. In yet another embodiment, the mmcE gene consists of the sequence of SEQ ID NO: 32.

In one embodiment, the at least one gene encoding the at least one propionate catabolism enzyme is mmcE. Accordingly, in one embodiment, the mmcE gene has at least about 80% identity with SEQ ID NO: 106. In one embodiment, the mmcE gene has at least about 90% identity with SEQ ID NO: 106. In one embodiment, the mmcE gene has at least about 95% identity with SEQ ID NO: 106. In one embodiment, the mmcE gene has at least about 85%, 86%, 87%, 88%, 89%, 90%, 91%, 92%, 93%, 94%, 95%, 96%, 97%, 98%, or 99% identity with SEQ ID NO: 106. In another embodiment, the mmcE gene comprises the sequence of SEQ ID NO: 106. In yet another embodiment, the mmcE gene consists of the sequence of SEQ ID NO: 106.

In one embodiment, the at least one gene encoding the at least one propionate catabolism enzyme is mutA. mutA encodes MutA, a methylmalonyl-CoA mutase small subunit. Accordingly, in one embodiment, the mutA gene has at least about 80% identity with SEQ ID NO: 33. In one embodiment, the mutA gene has at least about 90% identity with SEQ ID NO: 33. In one embodiment, the mutA gene has at least about 95% identity with SEQ ID NO: 33. In one embodiment, the mutA gene has at least about 85%, 86%, 87%, 88%, 89%, 90%, 91%, 92%, 93%, 94%, 95%, 96%, 97%, 98%, or 99% identity with SEQ ID NO: 33. In another embodiment, the mutA gene comprises the sequence of SEQ ID NO: 33. In yet another embodiment, the mutA gene consists of the sequence of SEQ ID NO: 33.

In one embodiment, the at least one gene encoding the at least one propionate catabolism enzyme is mutA. Accordingly, in one embodiment, the mutA gene has at least about 80% identity with SEQ ID NO: 110. In one embodiment, the mutA gene has at least about 90% identity with SEQ ID NO: 110. In one embodiment, the mutA gene has at least about 95% identity with SEQ ID NO: 110. In one embodiment, the mutA gene has at least about 85%, 86%, 87%, 88%, 89%, 90%, 91%, 92%, 93%, 94%, 95%, 96%, 97%, 98%, or 99% identity with SEQ ID NO: 110. In another embodiment, the mutA gene comprises the sequence of SEQ ID NO: 110. In yet another embodiment, the mutA gene consists of the sequence of SEQ ID NO: 110.

In one embodiment, the at least one gene encoding the at least one propionate catabolism enzyme is mutB. mutB encodes MutB, a methylmalonyl-CoA mutase large subunit. Accordingly, in one embodiment, the mutB gene has at least about 80% identity with SEQ ID NO: 34. In one embodiment, the mutB gene has at least about 90% identity with SEQ ID NO: 34. In one embodiment, the mutB gene has at least about 95% identity with SEQ ID NO: 34. In one embodiment, the mutB gene has at least about 85%, 86%, 87%, 88%, 89%, 90%, 91%, 92%, 93%, 94%, 95%, 96%, 97%, 98%, or 99% identity with SEQ ID NO: 34. In another embodiment, the mutB gene comprises the sequence of SEQ ID NO: 34. In yet another embodiment, the mutB gene consists of the sequence of SEQ ID NO: 34.

In one embodiment, the at least one gene encoding the at least one propionate catabolism enzyme is mutB. mutB encodes MutB, a methylmalonyl-CoA mutase large subunit. Accordingly, in one embodiment, the mutB gene has at least about 80% identity with SEQ ID NO: 112. In one embodiment, the mutB gene has at least about 90% identity with SEQ ID NO: 112. In one embodiment, the mutB gene has at least about 95% identity with SEQ ID NO: 112. In one embodiment, the mutB gene has at least about 85%, 86%, 87%, 88%, 89%, 90%, 91%, 92%, 93%, 94%, 95%, 96%, 97%, 98%, or 99% identity with SEQ ID NO: 112. In another embodiment, the mutB gene comprises the sequence of SEQ ID NO: 112. In yet another embodiment, the mutB gene consists of the sequence of SEQ ID NO: 112.

In one embodiment, the at least one gene encoding the at least one propionate catabolism enzyme is prpE. In one embodiment, the at least one propionate catabolism enzyme is prpE. In one embodiment, prpE has at least about 80% identity with SEQ ID NO: 71. In one embodiment, prpE has at least about 90% identity with SEQ ID NO: 71. In another embodiment, prpE has at least about 95% identity with SEQ ID NO: 71. Accordingly, in one embodiment, the prpE has at least about 85%, 86%, 87%, 88%, 89%, 90%, 91%, 92%, 93%, 94%, 95%, 96%, 97%, 98%, or 99% identity with SEQ ID NO: 71. In another embodiment, the prpE comprises a sequence which encodes SEQ ID NO: 71. In yet another embodiment, prpE consists of a sequence which encodes SEQ ID NO: 71.

In one embodiment, the at least one propionate catabolism enzyme is phaA. Accordingly, in one embodiment, phaA has at least about 80% identity with SEQ ID NO: 137. In one embodiment, phaA has at least about 90% identity with SEQ ID NO: 175. In another embodiment, phaA has at least about 95% identity with SEQ ID NO: 137. Accordingly, in one embodiment, phaA has at least about 85%, 86%, 87%, 88%, 89%, 90%, 91%, 92%, 93%, 94%, 95%, 96%, 97%, 98%, or 99% identity with SEQ ID NO: 137. In another embodiment, phaA comprises a sequence which encodes SEQ ID NO: 137. In yet another embodiment phaA consists of a sequence which encodes SEQ ID NO: 137.

In one embodiment, the at least one propionate catabolism enzyme is phaB. Accordingly, in one embodiment, phaB has at least about 80% identity with SEQ ID NO: 135. In one embodiment, phaB has at least about 90% identity with SEQ ID NO: 135. In another embodiment, phaB has at least about 95% identity with SEQ ID NO: 135. Accordingly, in one embodiment, phaB has at least about 85%, 86%, 87%, 88%, 89%, 90%, 91%, 92%, 93%, 94%, 95%, 96%, 97%, 98%, or 99% identity with SEQ ID NO: 135. In another embodiment, phaB comprises a sequence which encodes SEQ ID NO: 135. In yet another embodiment phaB consists of a sequence which encodes SEQ ID NO: 135.

In one embodiment, the at least one propionate catabolism enzyme is phaC. Accordingly, in one embodiment, phaC has at least about 80% identity with SEQ ID NO: 136. In one embodiment, phaC has at least about 90% identity with SEQ ID NO: 136. In another embodiment, phaC has at least about 95% identity with SEQ ID NO: 136. Accordingly, in one embodiment, phaC has at least about 85%, 86%, 87%, 88%, 89%, 90%, 91%, 92%, 93%, 94%, 95%, 96%, 97%, 98%, or 99% identity with SEQ ID NO: 136. In another embodiment, phaC comprises a sequence which encodes SEQ ID NO: 136. In yet another embodiment phaC consists of a sequence which encodes SEQ ID NO: 136.

In one embodiment, the at least one propionate catabolism enzyme is mmcE. Accordingly, in one embodiment, mmcE has at least about 80% identity with SEQ ID NO: 132. In one embodiment, mmcE has at least about 90% identity with SEQ ID NO: 132. In another embodiment, mmcE has at least about 95% identity with SEQ ID NO: 132. Accordingly, in one embodiment, mmcE has at least about 85%, 86%, 87%, 88%, 89%, 90%, 91%, 92%, 93%, 94%, 95%, 96%, 97%, 98%, or 99% identity with SEQ ID NO: 132. In another embodiment, mmcE comprises a sequence which encodes SEQ ID NO: 132. In yet another embodiment mmcE consists of a sequence which encodes SEQ ID NO: 132.

In one embodiment, the at least one propionate catabolism enzyme is mutA. Accordingly, in one embodiment, mutA has at least about 80% identity with SEQ ID NO: 133. In one embodiment, mutA has at least about 90% identity with SEQ ID NO: 133. In another embodiment, mutA has at least about 95% identity with SEQ ID NO: 133. Accordingly, in one embodiment, mutA has at least about 85%, 86%, 87%, 88%, 89%, 90%, 91%, 92%, 93%, 94%, 95%, 96%, 97%, 98%, or 99% identity with SEQ ID NO: 133. In another embodiment, mutA comprises a sequence which encodes SEQ ID NO: 133. In yet another embodiment mutA consists of a sequence which encodes SEQ ID NO: 133.

In one embodiment, the at least one propionate catabolism enzyme is mutB. Accordingly, in one embodiment, mutB has at least about 80% identity with SEQ ID NO: 134. In one embodiment, mutB has at least about 90% identity with SEQ ID NO: 134. In another embodiment, mutB has at least about 95% identity with SEQ ID NO: 134. Accordingly, in one embodiment, mutB has at least about 85%, 86%, 87%, 88%, 89%, 90%, 91%, 92%, 93%, 94%, 95%, 96%, 97%, 98%, or 99% identity with SEQ ID NO: 134. In another embodiment, mutB comprises a sequence which encodes SEQ ID NO: 134. In yet another embodiment mutB consists of a sequence which encodes SEQ ID NO: 134.

In one embodiment, the at least one propionate catabolism enzyme is accA. Accordingly, in one embodiment, accA has at least about 80% identity with SEQ ID NO: 130. In one embodiment, accA has at least about 90% identity with SEQ ID NO: 130. In another embodiment, accA has at least about 95% identity with SEQ ID NO: 130. Accordingly, in one embodiment, accA has at least about 85%, 86%, 87%, 88%, 89%, 90%, 91%, 92%, 93%, 94%, 95%, 96%, 97%, 98%, or 99% identity with SEQ ID NO: 130. In another embodiment, accA comprises a sequence which encodes SEQ ID NO: 130. In yet another embodiment the accA consists of a sequence which encodes SEQ ID NO: 130.

In one embodiment, the at least one propionate catabolism enzyme is pccB. Accordingly, in one embodiment, pccB has at least about 80% identity with SEQ ID NO: 131. In one embodiment, pccB has at least about 90% identity with SEQ ID NO: 131. In another embodiment, pccB has at least about 95% identity with SEQ ID NO: 131. Accordingly, in one embodiment, pccB has at least about 85%, 86%, 87%, 88%, 89%, 90%, 91%, 92%, 93%, 94%, 95%, 96%, 97%, 98%, or 99% identity with SEQ ID NO: 131. In another embodiment, pccB comprises a sequence which encodes SEQ ID NO: 131. In yet another embodiment, pccB consists of a sequence which encodes SEQ ID NO: 131.

In one embodiment, the at least one propionate catabolism enzyme is prpC. Accordingly, in one embodiment, prpC has at least about 80% identity with SEQ ID NO: 74. In one embodiment, prpC has at least about 90% identity with SEQ ID NO: 74. In another embodiment, prpC has at least about 95% identity with SEQ ID NO: 74. Accordingly, in one embodiment, prpC has at least about 85%, 86%, 87%, 88%, 89%, 90%, 91%, 92%, 93%, 94%, 95%, 96%, 97%, 98%, or 99% identity with SEQ ID NO: 74. In another embodiment, prpC comprises a sequence which encodes SEQ ID NO: 74. In yet another embodiment, prpC consists of a sequence which encodes SEQ ID NO: 74.

In one embodiment, the at least one propionate catabolism enzyme is prpD. Accordingly, in one embodiment, prpD has at least about 80% identity with SEQ ID NO: 77. In one embodiment, prpD has at least about 90% identity with SEQ ID NO: 77. In another embodiment, prpD has at least about 95% identity with SEQ ID NO: 77. Accordingly, in one embodiment, prpD has at least about 85%, 86%, 87%, 88%, 89%, 90%, 91%, 92%, 93%, 94%, 95%, 96%, 97%, 98%, or 99% identity with SEQ ID NO: 77. In another embodiment, prpD comprises a sequence which encodes SEQ ID NO: 77. In yet another embodiment, prpD consists of a sequence which encodes SEQ ID NO: 77.

In one embodiment, the at least one gene encoding the at least one propionate catabolism enzyme is MatB. MatB encodes Malonyl-coenzyme A (malonyl-CoA) synthetase (MatB). Accordingly, in one embodiment, the MatB gene has at least about 80% identity with SEQ ID NO: 141. Accordingly, in one embodiment, the MatB gene has at least about 90% identity with SEQ ID NO: 141. Accordingly, in one embodiment, the MatB gene has at least about 95% identity with SEQ ID NO: 141. Accordingly, in one embodiment, the MatB gene has at least about 85%, 86%, 87%, 88%, 89%, 90%, 91%, 92%, 93%, 94%, 95%, 96%, 97%, 98%, or 99% identity with SEQ ID NO: 141. In another embodiment, the MatB gene comprises the sequence of SEQ ID NO: 141. In yet another embodiment the MatB gene consists of the sequence of SEQ ID NO: 141.

In one embodiment, the at least one propionate catabolism enzyme is matB. Accordingly, in one embodiment, matB has at least about 80% identity with SEQ ID NO: 140. In one embodiment, matB has at least about 90% identity with SEQ ID NO: 140. In another embodiment, matB has at least about 95% identity with SEQ ID NO: 140. Accordingly, in one embodiment, matB has at least about 85%, 86%, 89%, 88%, 89%, 90%, 91%, 92%, 93%, 94%, 95%, 96%, 97%, 98%, or 99% identity with SEQ ID NO: 140. In another embodiment, matB comprises a sequence which encodes SEQ ID NO: 140. In yet another embodiment, matB consists of a sequence which encodes SEQ ID NO: 140.

In one embodiment, the at least one propionate catabolism enzyme is prpB. Accordingly, in one embodiment, prpB has at least about 80% identity with SEQ ID NO: 80. In one embodiment, prpB has at least about 90% identity with SEQ ID NO: 80. In another embodiment, prpB has at least about 95% identity with SEQ ID NO: 80. Accordingly, in one embodiment, prpB has at least about 85%, 86%, 87%, 88%, 89%, 90%, 91%, 92%, 93%, 94%, 95%, 96%, 97%, 98%, or 99% identity with SEQ ID NO: 80. In another embodiment, prpB comprises a sequence which encodes SEQ ID NO: 80. In yet another embodiment, prpB consists of a sequence which encodes SEQ ID NO: 80.

In one embodiment, any combination of propionate catabolism enzymes that effectively reduce the level of propionate and/or a metabolite thereof can be used. In one embodiment, any combination of propionate catabolism enzymes that effectively reduce levels of propionate, propionyl CoA, methylmalonate and/or methylmalonyl CoA in a subject can be used. In one embodiment, the at least one gene encoding the at least one propionate catabolism enzyme is prpBCD. In another embodiment, the at least one gene encoding the at least one propionate catabolism enzyme is prpBCDE. Using all four heterologous genes, for example, prpBCDE, is not necessary but allows excess propionate to be converted into succinate and pyruvate, feeding the Krebs cycle and benefiting the bacteria by increasing their growth. In another embodiment, the at least one gene encoding the at least one propionate catabolism enzyme is prpE, pccB, accA1, mmcE, mutA, and mutB. In another embodiment, the at least one gene encoding the at least one propionate catabolism enzyme is prpE, pccB, and accA1 under the control of a first inducible promoter, and mmcE, mutA, and mutB under the control of a second inducible promoter. In another embodiment, the at least one gene encoding the at least one propionate catabolism enzyme is prpE, phaB, phaC, and phaA.

In one embodiment, the propionate catabolism gene cassette comprises prpBCD. Accordingly, in one embodiment, the prpBCD operon has at least about 80% identity with SEQ ID NO: 138. In another embodiment, the prpBCD operon has at least about 80% identity with SEQ ID NO: 83 OR SEQ ID NO: 84. Accordingly, in one embodiment, the prpBCD operon has at least about 90% identity with SEQ ID NO: 138. In another embodiment, the prpBCD operon has at least about 90% identity with SEQ ID NO: 83 OR SEQ ID NO: 84. Accordingly, in one embodiment, the prpBCD operon has at least about 95% identity with SEQ ID NO: 138. In another embodiment, the prpBCD operon has at least about 95% identity with SEQ ID NO: 83 OR SEQ ID NO: 84. Accordingly, in one embodiment, the prpBCD operon has at least about 85%, 86%, 87%, 88%, 89%, 90%, 91%, 92%, 93%, 94%, 95%, 96%, 97%, 98%, or 99% identity with SEQ ID NO: 138. In another embodiment, the prpBCD operon has at least about 85%, 86%, 87%, 88%, 89%, 90%, 91%, 92%, 93%, 94%, 95%, 96%, 97%, 98%, or 99% identity with SEQ ID NO: 83 OR SEQ ID NO: 84. In another embodiment, the prpBCD operon comprises the sequence of SEQ ID NO: 138. In another embodiment, the prpBCD operon comprises the sequence of SEQ ID NO: 83 OR SEQ ID NO: 84. In yet another embodiment the prpBCD operon consists of the sequence of SEQ ID NO: 138. In another embodiment, the prpBCD operon consists of the sequence of SEQ ID NO: 83 OR SEQ ID NO: 84.

In one embodiment, the propionate catabolism gene cassette comprises prpBCDE. Accordingly, in one embodiment, the prpBCDE operon has at least about 80% identity with SEQ ID NO: 55. In another embodiment, the prpBCDE operon has at least about 80% identity with SEQ ID NO: 93 or SEQ ID NO: 94. Accordingly, in one embodiment, the prpBCDE operon has at least about 90% identity with SEQ ID NO: 55. In another embodiment, the prpBCDE operon has at least about 90% identity with SEQ ID NO: 93 or SEQ ID NO: 94. Accordingly, in one embodiment, the prpBCDE operon has at least about 95% identity with SEQ ID NO: 55. In another embodiment, the prpBCDE operon has at least about 95% identity with SEQ ID NO: 93 or SEQ ID NO: 94. Accordingly, in one embodiment, the prpBCDE operon has at least about 85%, 86%, 87%, 88%, 89%, 90%, 91%, 92%, 93%, 94%, 95%, 96%, 97%, 98%, or 99% identity with SEQ ID NO: 55. In another embodiment, the prpBCDE operon has at least about 85%, 86%, 87%, 88%, 89%, 90%, 91%, 92%, 93%, 94%, 95%, 96%, 97%, 98%, or 99% identity with SEQ ID NO: 93 or SEQ ID NO: 94. In another embodiment, the prpBCDE operon comprises the sequence of SEQ ID NO: 55. In another embodiment, the prpBCDE operon comprises the sequence of SEQ ID NO: 93 or SEQ ID NO: 94. In yet another embodiment the prpBCDE operon consists of the sequence of SEQ ID NO: 55. In another embodiment, the prpBCDE operon consists of the sequence of SEQ ID NO: 93 or SEQ ID NO: 94.

In one embodiment, the propionate catabolism gene cassette comprises phaBCA. Accordingly, in one embodiment, the phaBCA operon has at least about 80% identity with SEQ ID NO: 139. In one embodiment, the phaBCA operon has at least about 90% identity with SEQ ID NO: 139. In one embodiment, the phaBCA operon has at least about 95% identity with SEQ ID NO: 139. In one embodiment, the phaBCA operon has at least about 85%, 86%, 87%, 88%, 89%, 90%, 91%, 92%, 93%, 94%, 95%, 96%, 97%, 98%, or 99% identity with SEQ ID NO: 139. In another embodiment, the phaBCA operon comprises the sequence of SEQ ID NO: 139. In another embodiment, the phaBCA operon consists of the sequence of SEQ ID NO: 139. In one embodiment, the propionate catabolism gene cassette comprises prpE and phaBCA.

In one embodiment, the propionate catabolism gene cassette comprises phaBCA. Accordingly, in one embodiment, the phaBCA operon has at least about 80% identity with SEQ ID NO: 102. In one embodiment, the phaBCA operon has at least about 90% identity with SEQ ID NO: 102. In one embodiment, the phaBCA operon has at least about 95% identity with SEQ ID NO: 102. In one embodiment, the phaBCA operon has at least about 85%, 86%, 87%, 88%, 89%, 90%, 91%, 92%, 93%, 94%, 95%, 96%, 97%, 98%, or 99% identity with SEQ ID NO: 102. In another embodiment, the phaBCA operon comprises the sequence of SEQ ID NO: 102. In another embodiment, the phaBCA operon consists of the sequence of SEQ ID NO: 102. In one embodiment, the propionate catabolism gene cassette comprises prpE and phaBCA.

In one embodiment, the propionate catabolism gene cassette comprises prpE-phaBCA. Accordingly, in one embodiment, the prpE-phaBCA operon has at least about 80% identity with SEQ ID NO: 24. In one embodiment, the prpE-phaBCA operon has at least about 90% identity with SEQ ID NO: 24. In one embodiment, the prpE-phaBCA operon has at least about 95% identity with SEQ ID NO: 24. In one embodiment, the prpE-phaBCA operon has at least about 85%, 86%, 87%, 88%, 89%, 90%, 91%, 92%, 93%, 94%, 95%, 96%, 97%, 98%, or 99% identity with SEQ ID NO: 24. In another embodiment, the prpE-phaBCA operon comprises the sequence of SEQ ID NO: 24. In another embodiment, the prpE-phaBCA operon consists of the sequence of SEQ ID NO: 24.

In one embodiment, the propionate catabolism gene cassette comprises prpE, pccB, accA1, mmcE, mutA, and mutB. Accordingly, in one embodiment, the prpE-pccB-accA1-mmcE-mutA-mutB operon has at least about 80% identity with a combination of SEQ ID NO: 37 and 31. In one embodiment, the prpE-pccB-accA1-mmcE-mutA-mutB operon has at least about 90% identity with a combination of SEQ ID NO: 37 and 31. In one embodiment, the prpE-pccB-accA1-mmcE-mutA-mutB operon has at least about 95% identity with a combination of SEQ ID NO: 37 and 31. In one embodiment, the prpE-pccB-accA1-mmcE-mutA-mutB operon has at least about 85%, 86%, 87%, 88%, 89%, 90%, 91%, 92%, 93%, 94%, 95%, 96%, 97%, 98%, or 99% identity with a combination of SEQ ID NO: 37 and 31. In another embodiment, the prpE-pccB-accA1-mmcE-mutA-mutB operon comprises the sequence of a combination of SEQ ID NO: 37 and 31. In another embodiment, the prpE-pccB-accA1-mmcE-mutA-mutB operon consists of the sequence of a combination of SEQ ID NO: 37 and 31.

In one embodiment, the propionate catabolism gene cassette comprises prpE, pccB, and accA1. Accordingly, in one embodiment, the prpE-pccB-accA1 operon has at least about 80% identity with SEQ ID NO: 37. In one embodiment, the prpE-pccB-accA1 operon has at least about 90% identity with SEQ ID NO: 37. In one embodiment, the prpE-pccB-accA1 operon has at least about 95% identity with SEQ ID NO: 37. In one embodiment, the prpE-pccB-accA1 operon has at least about 85%, 86%, 87%, 88%, 89%, 90%, 91%, 92%, 93%, 94%, 95%, 96%, 97%, 98%, or 99% identity with SEQ ID NO: 37. In another embodiment, the prpE-pccB-accA1 operon comprises the sequence of SEQ ID NO: 37. In another embodiment, the prpE-pccB-accA1 operon consists of the sequence of SEQ ID NO: 37.

In one embodiment, the propionate catabolism gene cassette comprises mmcE, mutA, and mutB. Accordingly, in one embodiment, the mmcE-mutA-mutB operon has at least about 80% identity with a combination of SEQ ID NO:31. In one embodiment, the mmcE-mutA-mutB operon has at least about 90% identity with a combination of SEQ ID NO: 31. In one embodiment, the -mmcE-mutA-mutB operon has at least about 95% identity with a combination of SEQ ID NO: 31. In one embodiment, the mmcE-mutA-mutB operon has at least about 85%, 86%, 87%, 88%, 89%, 90%, 91%, 92%, 93%, 94%, 95%, 96%, 97%, 98%, or 99% identity with a combination of SEQ ID NO: 31. In another embodiment, the mmcE-mutA-mutB operon comprises the sequence of a combination of SEQ ID NO: 31. In another embodiment, the mmcE-mutA-mutB operon consists of the sequence of a combination of SEQ ID NO: 31.

In one embodiment, the at least one gene encoding the at least one propionate catabolism enzyme is directly operably linked to a first promoter. In another embodiment, the at least one gene encoding the at least one propionate catabolism enzyme is indirectly operably linked to a first promoter. In one embodiment, the promoter is not operably linked with the at least one gene encoding the propionate catabolism enzyme in nature.

In some embodiments, the at least one gene encoding the at least one propionate catabolism enzyme is expressed under the control of a constitutive promoter. In another embodiment, the at least one gene encoding the at least one propionate catabolism enzyme is expressed under the control of an inducible promoter. In some embodiments, the at least one gene encoding the at least one propionate catabolism enzyme is expressed under the control of a promoter that is directly or indirectly induced by exogenous environmental conditions. In one embodiment, the at least one gene encoding the at least one propionate catabolism enzyme is expressed under the control of a promoter that is directly or indirectly induced by low-oxygen or anaerobic conditions, wherein expression of the at least one gene encoding the at least one propionate catabolism enzyme is activated under low-oxygen or anaerobic environments, such as the environment of the mammalian gut. Inducible promoters are described in more detail infra.

The at least one gene encoding the at least one propionate catabolism enzyme may be present on a plasmid or chromosome in the bacterial cell. In one embodiment, the at least one gene encoding the at least one propionate catabolism enzyme is located on a plasmid in the bacterial cell. In another embodiment, the at least one gene encoding the at least one propionate catabolism enzyme is located in the chromosome of the bacterial cell. In yet another embodiment, a native copy of the at least one gene encoding the at least one propionate catabolism enzyme is located in the chromosome of the bacterial cell, and at least one gene encoding at least one propionate catabolism enzyme from a different species of bacteria is located on a plasmid in the bacterial cell. In yet another embodiment, a native copy of the at least one gene encoding the at least one propionate catabolism enzyme is located on a plasmid in the bacterial cell, and at least one gene encoding the at least one propionate catabolism enzyme from a different species of bacteria is located on a plasmid in the bacterial cell. In yet another embodiment, a native copy of the at least one gene encoding the at least one propionate catabolism enzyme is located in the chromosome of the bacterial cell, and at least one gene encoding the at least one propionate catabolism enzyme from a different species of bacteria is located in the chromosome of the bacterial cell.

In some embodiments, the at least one gene encoding the at least one propionate catabolism enzyme is expressed on a low-copy plasmid. In some embodiments, the at least one gene encoding the at least one propionate catabolism enzyme is expressed on a high-copy plasmid. In some embodiments, the high-copy plasmid may be useful for increasing expression of the at least one propionate catabolism enzyme, thereby increasing the catabolism of propionate, propionic acid, propionyl CoA, methylmalonic acid, and/or methylmalonyl CoA.

In some embodiments, a engineered bacterial cell comprising at least one gene encoding at least one propionate catabolism enzyme expressed on a high-copy plasmid does not increase propionate catabolism or decrease propionate, propionyl CoA, methylmalonate and/or methylmalonyl CoA levels as compared to a engineered bacterial cell comprising the same gene expressed on a low-copy plasmid in the absence of a heterologous importer of propionate and additional copies of a native importer of propionate. It has been surprisingly discovered that in some embodiments, the rate-limiting step of propionate catabolism is not expression of a propionate catabolism enzyme, but rather availability of propionate or propionyl CoA. Thus, in some embodiments, it may be advantageous to increase propionate transport into the cell, thereby enhancing propionate catabolism. Furthermore, in some embodiments that incorporate a transporter of propionate into the engineered bacterial cell, there may be additional advantages to using a low-copy plasmid comprising the at least one gene encoding the at least one propionate catabolism enzyme in conjunction in order to enhance the stability of expression of the propionate catabolism enzyme, while maintaining high propionate catabolism and to reduce negative selection pressure on the transformed bacterium. In alternate embodiments, the importer of propionate is used in conjunction with a high-copy plasmid.

Deacylation of propionylated PrpE (PrpE^{Pr}r) by CobB, a NAD-dependent deacylase, allows bacterial cells to catabolize propionate. Thus, in one embodiment, when the engineered bacterial cell expresses a heterologous PrpE enzyme, the engineered bacterial cell may further comprise a heterologous cobB gene (SEQ ID NO:114). In one embodiment, the cobB gene has at least about 80% identity with SEQ ID NO: 114. Accordingly, in one embodiment, the cobB gene has at least about 90% identity with SEQ ID NO: 114. Accordingly, in one embodiment, the cobB gene has at least about 95% identity with SEQ ID NO: 114. Accordingly, in one embodiment, the cobB gene has at least about 85%, 86%, 87%, 88%, 89%, 90%, 91%, 92%, 93%, 94%, 95%, 96%, 97%, 98%, or 99% identity with SEQ ID NO: 114. In another embodiment, the cobB gene comprises the sequence of SEQ ID NO: 114. In yet another embodiment the cobB gene consists of the sequence of SEQ ID NO: 114.

In one embodiment, the at least one propionate catabolism enzyme is CobB. Accordingly, in one embodiment, CobB has at least about 113% identity with SEQ ID NO: 113. In one embodiment, CobB has at least about 90% identity with SEQ ID NO: 113. In another embodiment, CobB has at least about 95% identity with SEQ ID NO: 113. Accordingly, in one embodiment, CobB has at least about 85%, 86%, 87%, 88%, 89%, 90%, 91%, 92%, 93%, 94%, 95%, 96%, 97%, 98%, or 99% identity with SEQ ID NO: 113. In another embodiment, CobB comprises a sequence which encodes SEQ ID NO: 113. In yet another embodiment, CobB consists of a sequence which encodes SEQ ID NO: 113.

In another embodiment, the engineered bacterial cell comprising a heterologous cobB gene further comprises a genetic modification in the pka gene. Pka, a protein lysine acetyltransferase, renders PrpE in the propionylated form (PrpE^{P}r) unable to metabolize propionate. Therefore, genetic modification of the pka gene (SEQ ID NO: 116) which renders it functionally inactive enhances the ability of the bacterial cells to catabolize propionate.

Transporter (Importer) of Propionate

The uptake of propionate into bacterial cells typically occurs via passive diffusion (see, for example, Kell et al., 1981, Biochem. Biophys. Res. Commun., 9981-9988). However, the active import of propionate is also mediated by proteins well known to those of skill in the art. For example, a bacterial transport system for the update of propionate in Corynebacterium glutamicum named MctC (monocarboxylic acid transporter) is known (see, for example, Jolkver et al., 2009, J. Bacteriol., 191(3):940-948). The putP_6 propionate transporter from Virgibacillus species (UniProt A0A024QGU1) has also been identified.

Propionate transporters, e.g., propionate importers, may be expressed or modified in the bacteria in order to enhance propionate transport into the cell. Specifically, when the transporter (importer) of propionate is expressed in the engineered bacterial cells, the bacterial cells import more propionate into the cell when the transporter is expressed than unmodified bacteria of the same bacterial subtype under the same conditions. Thus, the genetically engineered bacteria comprising a heterologous gene encoding a transporter of propionate may be used to import propionate into the bacteria so that any gene encoding a propionate catabolism enzyme expressed in the organism can be used to treat diseases associated with the catabolism of propionate, such as organic acidurias (including PA and MMA) and vitamin B₁₂ deficiencies. In one embodiment, the bacterial cell comprises a heterologous gene encoding a transporter of propionate. In one embodiment, the bacterial cell comprises a heterologous gene encoding a transporter of propionate and at least one heterologous gene encoding at least one propionate catabolism enzyme.

Thus, in some embodiments, the disclosure provides a bacterial cell that comprises at least one heterologous gene encoding a propionate catabolism enzyme operably linked to a first promoter and at least one heterologous gene encoding a propionate transporter. In some embodiments, the disclosure provides a bacterial cell that comprises at least one heterologous gene encoding a transporter of propionate operably linked to the first promoter. In another embodiment, the disclosure provides a bacterial cell that comprises at least one heterologous gene encoding at least one propionate catabolism enzyme operably linked to a first promoter and at least one heterologous gene encoding of propionate operably linked to a second promoter. In one embodiment, the first promoter and the second promoter are separate copies of the same promoter. In another embodiment, the first promoter and the second promoter are different promoters.

In one embodiment, the bacterial cell comprises at least one gene encoding a transporter of propionate from a different organism, e.g., a different species of bacteria. In one embodiment, the bacterial cell comprises at least one native gene encoding a transporter of propionate. In some embodiments, the at least one native gene encoding a transporter of propionate is not modified. In another embodiment, the bacterial cell comprises more than one copy of at least one native gene encoding a transporter of propionate. In yet another embodiment, the bacterial cell comprises a copy of at least one gene encoding a native importer of propionate, as well as at least one copy of at least one heterologous gene encoding a transporter of propionate from a different bacterial species. In one embodiment, the bacterial cell comprises at least one, two, three, four, five, or six copies of the at least one heterologous gene encoding a transporter of propionate. In one embodiment, the bacterial cell comprises multiple copies of the at least one heterologous gene encoding a transporter of propionate.

In some embodiments, the importer of propionate is encoded by a transporter of propionate gene derived from a bacterial genus or species, including but not limited to, Bacillus, Campylobacter, Clostridium, Corynebacterium, Escherichia, Lactobacillus, Pseudomonas, Salmonella, Staphylococcus, Bacillus subtilis, Campylobacter jejuni, Clostridium perfringens, Escherichia coli, Lactobacillus delbrueckii, Pseudomonas aeruginosa, Salmonella typhimurium, Virgibacillus, or Staphylococcus aureus. In some embodiments, the bacterium is a Virgibacillus. In some embodiments, the bacterial is a Corynebacterium. In one embodiment, the bacterium is C. glutamicum. In another embodiment, the bacterium is C. diphtheria. In another embodiment, the bacterium is C. efficiens. In another embodiment, the bacterium is S. coelicolor. In another embodiment, the bacterium is M. smegmatis. In another embodiment, the bacterium is N. farcinica. In another embodiment, the bacterium is E. coli. In another embodiment, the bacterium is B. subtilis.

The present disclosure further comprises genes encoding functional fragments of a transporter of propionate or functional variants of a transporter of propionate. As used herein, the term “functional fragment thereof” or “functional variant thereof” of a transporter of propionate relates to an element having qualitative biological activity in common with the wild-type importer of propionate from which the fragment or variant was derived. For example, a functional fragment or a functional variant of a mutated importer of propionate protein is one which retains essentially the same ability to import propionate into the bacterial cell as does the importer protein from which the functional fragment or functional variant was derived. In one embodiment, the engineered bacterial cell comprises at least one heterologous gene encoding a functional fragment of a transporter of propionate. In another embodiment, the engineered bacterial cell comprises at least one heterologous gene encoding a functional variant of a transporter of propionate.

Assays for testing the activity of a transporter of propionate, a transporter of propionate functional variant, or a transporter of propionate functional fragment are well known to one of ordinary skill in the art. For example, propionate import can be assessed by expressing the protein, functional variant, or fragment thereof, in engineered bacterial cell that lacks an endogenous propionate importer. Propionate import can also be assessed using mass spectrometry. Propionate import can also be expressed using gas chromatography. For example, samples can be injected into a Perkin Elmer Autosystem XL Gas Chromatograph containing a Supelco packed column, and the analysis can be performed according to manufacturing instructions (see, for example, Supelco I (1998) Analyzing fatty acids by packed column gas chromatography, Bulletin 856B:2014). Alternatively, samples can be analyzed for propionate import using high-pressure liquid chromatography (HPLC). For example, a computer-controlled Waters HPLC system equipped with a model 600 quaternary solvent delivery system, and a model 996 photodiode array detector, and components of the sample can be resolved with an Aminex HPX-87H (300 by 7.8 mm) organic acid analysis column (Bio-Rad Laboratories) (see, for example, Palacios et al., 2003, J. Bacteriol., 185(9):2802-2810).

In one embodiment, the genes encoding the importer of propionate have been codon-optimized for use in the host organism. In one embodiment, the genes encoding the importer of propionate have been codon-optimized for use in Escherichia coli.

The present disclosure also encompasses genes encoding a transporter of propionate comprising amino acids in its sequence that are substantially the same as an amino acid sequence described herein Amino acid sequences that are substantially the same as the sequences described herein include sequences comprising conservative amino acid substitutions, as well as amino acid deletions and/or insertions.

In some embodiments, the at least one gene encoding a transporter of propionate is mutagenized; mutants exhibiting increased propionate transport are selected; and the mutagenized at least one gene encoding a transporter of propionate is isolated and inserted into the bacterial cell. In some embodiments, the at least one gene encoding a transporter of propionate is mutagenized; mutants exhibiting decreased propionate transport are selected; and the mutagenized at least one gene encoding a transporter of propionate is isolated and inserted into the bacterial cell. The importer modifications described herein may be present on a plasmid or chromosome.

In one embodiment, the propionate importer is MctC. In one embodiment, the mctC gene has at least about 80% identity to SEQ ID NO: 88. Accordingly, in one embodiment, the mctC gene has at least about 90% identity to SEQ ID NO: 88. Accordingly, in one embodiment, the mctC gene has at least about 95% identity to SEQ ID NO: 88. Accordingly, in one embodiment, the mctC gene has at least about 85%, 86%, 87%, 88%, 89%, 90%, 91%, 92%, 93%, 94%, 95%, 96%, 97%, 98%, or 99% identity to SEQ ID NO: 88. In another embodiment, the mctC gene comprises the sequence of SEQ ID NO: 88. In yet another embodiment the mctC gene consists of the sequence of SEQ ID NO: 88.

In one embodiment, the at least one propionate catabolism enzyme is MctC. Accordingly, in one embodiment, MctC has at least about 80% identity with SEQ ID NO: 87. In one embodiment, MctC has at least about 90% identity with SEQ ID NO: 87. In another embodiment, MctC has at least about 95% identity with SEQ ID NO: 87. Accordingly, in one embodiment, MctC has at least about 85%, 86%, 87%, 88%, 89%, 90%, 91%, 92%, 93%, 94%, 95%, 96%, 97%, 98%, or 99% identity with SEQ ID NO: 87. In another embodiment, MctC comprises a sequence which encodes SEQ ID NO: 87. In yet another embodiment, MctC consists of a sequence which encodes SEQ ID NO: 87.

In another embodiment, the propionate importer is PutP_6. In one embodiment, the putP_6 gene has at least about 80% identity to SEQ ID NO: 90. Accordingly, in one embodiment, the putP_6 gene has at least about 90% identity to SEQ ID NO: 90. Accordingly, in one embodiment, the putP_6 gene has at least about 95% identity to SEQ ID NO: 90. Accordingly, in one embodiment, the putP_6 gene has at least about 85%, 86%, 87%, 88%, 89%, 90%, 91%, 92%, 93%, 94%, 95%, 96%, 97%, 98%, or 99% identity to SEQ ID NO: 90. In another embodiment, the putP_6 gene comprises the sequence of SEQ ID NO: 90. In yet another embodiment the putP_6 gene consists of the sequence of SEQ ID NO: 90.

In one embodiment, the at least one propionate catabolism enzyme is PutP_6. Accordingly, in one embodiment, PutP_6 has at least about 80% identity with SEQ ID NO: 89. In one embodiment, PutP_6 has at least about 90% identity with SEQ ID NO: 89. In another embodiment, PutP_6 has at least about 95% identity with SEQ ID NO: 89. Accordingly, in one embodiment, PutP_6 has at least about 85%, 86%, 89%, 88%, 89%, 90%, 91%, 92%, 93%, 94%, 95%, 96%, 97%, 98%, or 99% identity with SEQ ID NO: 89. In another embodiment, PutP_6 comprises a sequence which encodes SEQ ID NO: 89. In yet another embodiment, PutP_6 consists of a sequence which encodes SEQ ID NO: 89.

Other propionate importer genes are known to those of ordinary skill in the art. See, for example, Jolker et al., J. Bacteria, 2009, 191(3):940-948. In one embodiment, the propionate importer comprises the mctBC genes from C. glutamicum. In another embodiment, the propionate importer comprises the dip0780 and dip0791 genes from C. diphtheria. In another embodiment, the propionate importer comprises the ce0909 and ce0910 genes from C. efficiens. In another embodiment, the propionate importer comprises the ce1091 and ce1092 genes from C. efficiens. In another embodiment, the propionate importer comprises the sco1822 and sco1823 genes from S. coelicolor. In another embodiment, the propionate importer comprises the sco1218 and sco1219 genes from S. coelicolor. In another embodiment, the propionate importer comprises the eel 091 and sco5827 genes from S. coelicolor. In another embodiment, the propionate importer comprises the m_5160, m_5161, m_5165, and m_5166 genes from M. smegmatis. In another embodiment, the propionate importer comprises the nfa 17930, nfa 17940, nfa 17950, and nfa 17960 genes from N. farcinica. In another embodiment, the propionate importer comprises the actP and yjcH genes from E. coli. In another embodiment, the propionate importer comprises the ywcB and ywcA genes from B. subtilis.

In some embodiments, the bacterial cell comprises at least one heterologous gene encoding at least one propionate catabolism enzyme operably linked to a first promoter and at least one heterologous gene encoding a transporter of propionate. In some embodiments, the at least one heterologous gene encoding a transporter of propionate is operably linked to the first promoter. In other embodiments, the at least one heterologous gene encoding a transporter of propionate is operably linked to a second promoter. In one embodiment, the at least one gene encoding a transporter of propionate is directly operably linked to the second promoter. In another embodiment, the at least one gene encoding a transporter of propionate is indirectly operably linked to the second promoter.

In some embodiments, expression of at least one gene encoding a transporter of propionate is controlled by a different promoter than the promoter that controls expression of the at least one gene encoding the at least one propionate catabolism enzyme. In some embodiments, expression of the at least one gene encoding a transporter of propionate is controlled by the same promoter that controls expression of the at least one propionate catabolism enzyme. In some embodiments, at least one gene encoding a transporter of propionate and the propionate catabolism enzyme are divergently transcribed from a promoter region. In some embodiments, expression of each of genes encoding the at least one gene encoding a transporter of propionate and the at least one gene encoding the at least one propionate catabolism enzyme is controlled by different promoters.

In one embodiment, the promoter is not operably linked with the at least one gene encoding a transporter of propionate in nature. In some embodiments, the at least one gene encoding the importer of propionate is controlled by its native promoter. In some embodiments, the at least one gene encoding the importer of propionate is controlled by an inducible promoter. In some embodiments, the at least one gene encoding the importer of propionate is controlled by a promoter that is stronger than its native promoter. In some embodiments, the at least one gene encoding the importer of propionate is controlled by a constitutive promoter.

In another embodiment, the promoter is an inducible promoter. Inducible promoters are described in more detail infra.

In one embodiment, the at least one gene encoding a transporter of propionate is located on a plasmid in the bacterial cell. In another embodiment, the at least one gene encoding a transporter of propionate is located in the chromosome of the bacterial cell. In yet another embodiment, a native copy of the at least one gene encoding a transporter of propionate is located in the chromosome of the bacterial cell, and a copy of at least one gene encoding a transporter of propionate from a different species of bacteria is located on a plasmid in the bacterial cell. In yet another embodiment, a native copy of the at least one gene encoding a transporter of a propionate is located on a plasmid in the bacterial cell, and a copy of at least one gene encoding a transporter of propionate from a different species of bacteria is located on a plasmid in the bacterial cell. In yet another embodiment, a native copy of the at least one gene encoding a transporter of propionate is located in the chromosome of the bacterial cell, and a copy of the at least one gene encoding a transporter of propionate from a different species of bacteria is located in the chromosome of the bacterial cell.

In some embodiments, the at least one native gene encoding the importer in the bacterial cell is not modified, and one or more additional copies of the native importer are inserted into the genome. In one embodiment, the one or more additional copies of the native importer that is inserted into the genome are under the control of the same inducible promoter that controls expression of the at least one gene encoding the propionate catabolism enzyme, e.g., the FNR responsive promoter, or a different inducible promoter than the one that controls expression of the at least one propionate catabolism enzyme, or a constitutive promoter. In alternate embodiments, the at least one native gene encoding the importer is not modified, and one or more additional copies of the importer from a different bacterial species is inserted into the genome of the bacterial cell. In one embodiment, the one or more additional copies of the importer inserted into the genome of the bacterial cell are under the control of the same inducible promoter that controls expression of the at least one gene encoding the propionate catabolism enzyme, e.g., the FNR responsive promoter, or a different inducible promoter than the one that controls expression of the at least one gene encoding the at least one propionate catabolism enzyme, or a constitutive promoter.

In one embodiment, when the importer of propionate is expressed in the engineered bacterial cells, the bacterial cells import 10% more propionate into the bacterial cell when the importer is expressed than unmodified bacteria of the same bacterial subtype under the same conditions. In another embodiment, when the importer of propionate is expressed in the engineered bacterial cells, the bacterial cells import 20%, 30%, 40%, 50%, 60%, 70%, 80%, 90% or 100% more propionate into the bacterial cell when the importer is expressed than unmodified bacteria of the same bacterial subtype under the same conditions. In yet another embodiment, when the importer of propionate is expressed in the engineered bacterial cells, the bacterial cells import two-fold more propionate into the cell when the importer is expressed than unmodified bacteria of the same bacterial subtype under the same conditions. In yet another embodiment, when the importer of propionate is expressed in the engineered bacterial cells, the bacterial cells import three-fold, four-fold, five-fold, six-fold, seven-fold, eight-fold, nine-fold, or ten-fold more propionate into the cell when the importer is expressed than unmodified bacteria of the same bacterial subtype under the same conditions.

Exporters of Succinate

Succinate export in bacteria is normally active under anaerobic conditions. The export of succinate is mediated by proteins well known to those of skill in the art. For example, a succinate exporter in Corynebacterium glutamicum is known as SucE1. SucE1 is a membrane protein belonging to the aspartate:alanine exchanger (AAE) family (see, for example, Fukui et al., 2011, J. Bacteriol., 154(1):25-34). The DcuC succinate exporter from E. coli has also been identified (see, for example, Cheng et al., 2013, J. Biomed. Res. Int, 2013:ID 538790).

Succinate transporters, e.g., succinate exporters, may be expressed or modified in the bacteria in order to enhance succinate export out of the cell. Specifically, when the exporter of succinate is expressed in the engineered bacterial cells, the bacterial cells export more succinate outside of the cell when the exporter is expressed than unmodified bacteria of the same bacterial subtype under the same conditions. In one embodiment, the bacterial cell comprises a heterologous gene encoding an exporter of succinate. In one embodiment, the bacterial cell comprises a heterologous gene encoding an exporter of succinate and at least one heterologous gene or gene cassette encoding at least one propionate catabolism enzyme.

Thus, in some embodiments, the disclosure provides a bacterial cell that comprises at least one heterologous gene or gene cassette encoding a propionate catabolism enzyme or enzymes operably linked to a first promoter and at least one heterologous gene encoding an exporter of succinate. In some embodiments, the at least one heterologous gene encoding an exporter of succinate is operably linked to the first promoter. In another embodiment, the at least one heterologous gene encoding the at least one propionate catabolism enzyme operably is linked to a first promoter, and the heterologous gene encoding an exporter of succinate is operably linked to a second promoter. In one embodiment, the first promoter and the second promoter are separate copies of the same promoter. In another embodiment, the first promoter and the second promoter are different promoters.

In one embodiment, the bacterial cell comprises at least one gene encoding an exporter of succinate from a different organism, e.g., a different species of bacteria. In one embodiment, the bacterial cell comprises at least one native gene encoding an exporter of succinate. In some embodiments, the at least one native gene encoding an exporter of succinate is not modified. In another embodiment, the bacterial cell comprises more than one copy of at least one native gene encoding an exporter of succinate. In yet another embodiment, the bacterial cell comprises a copy of at least one gene encoding a native exporter of succinate, as well as at least one copy of at least one heterologous gene encoding an exporter of succinate from a different bacterial species. In one embodiment, the bacterial cell comprises at least one, two, three, four, five, or six copies of the at least one heterologous genes encoding an exporter of succinate. In one embodiment, the bacterial cell comprises multiple copies of the at least one heterologous gene encoding an exporter of succinate.

In some embodiments, the exporter of succinate is encoded by an exporter of succinate gene derived from a bacterial genus or species, including but not limited to, Actinobacillus succinogenes, Anaerobiospirillum succiniciproducens, and Mannheimia succiniciproducens, Escherichia coli, Corynebacterium glutamicum, Salmonella typhimurium, Klebsiella pneumoniae, Serratia plymuthica, Enterobacter cloacae, Bacillus subtilis, Bacillus anthracia, bacillus lichenformis, and Saccharomyces cerevisiae. In some embodiments, the exporter of succinate is derived from Corynebacterium. In one embodiment, the exporter of succinate is derived from C. glutamicum. In another embodiment, the exporter of succinate is from Vibrio cholerae. In another embodiment, the exporter of succinate is from E. coli. In another embodiment, the exporter of succinate is from Bacillus subtilis.

The present disclosure further comprises genes encoding functional fragments of an exporter of succinate or functional variants of an exporter of succinate. As used herein, the term “functional fragment thereof” or “functional variant thereof” of an exporter of succinate relates to an element having qualitative biological activity in common with the wild-type exporter of succinate from which the fragment or variant was derived. For example, a functional fragment or a functional variant of a mutated exporter of succinate protein is one which retains essentially the same ability to import succinate into the bacterial cell as does the exporter protein from which the functional fragment or functional variant was derived. In one embodiment, the engineered bacterial cell comprises at least one heterologous gene encoding a functional fragment of an exporter of succinate. In another embodiment, the engineered bacterial cell comprises at least one heterologous gene encoding a functional variant of an exporter of succinate.

In some embodiments, the genetically engineered bacteria further comprise a mutation or deletion in one or more succinate importers, e.g., Dct, DctC, ybhI or ydjN. In some embodiments, succinate dehydrogenase (SUCDH) may be mutated or deleted. Without wishing to be bound by theory, such mutations may decrease intracellular succinate concentrations and increase the flux through propionate catabolism pathways.

Assays for testing the activity of an exporter of succinate, an exporter of succinate functional variant, or an exporter of succinate functional fragment are well known to one of ordinary skill in the art. For example, succinate export can be assessed by expressing the protein, functional variant, or fragment thereof, in a engineered bacterial cell that lacks an endogenous succinate exporter and assessing succinate levels in the media after expression of the protein. Methods for measuring succinate export are well known to one of ordinary skill in the art. For example, see Fukui et al., J. Biotechnol., 154(1):25-34, 2011.

In one embodiment, the genes encoding the exporter of succinate have been codon-optimized for use in the host organism. In one embodiment, the genes encoding the exporter of succinate have been codon-optimized for use in Escherichia coli.

The present disclosure also encompasses genes encoding an exporter of succinate comprising amino acids in its sequence that are substantially the same as an amino acid sequence described herein Amino acid sequences that are substantially the same as the sequences described herein include sequences comprising conservative amino acid substitutions, as well as amino acid deletions and/or insertions.

In some embodiments, the at least one gene encoding an exporter of succinate is mutagenized; mutants exhibiting increased succinate transport are selected; and the mutagenized at least one gene encoding an exporter of succinate is isolated and inserted into the bacterial cell. In some embodiments, the at least one gene encoding an exporter of succinate is mutagenized; mutants exhibiting decreased succinate transport are selected; and the mutagenized at least one gene encoding an exporter of succinate is isolated and inserted into the bacterial cell. The exporter modifications described herein may be present on a plasmid or chromosome.

In one embodiment, the succinate exporter is DcuC. In one embodiment, the dcuC gene has at least about 80% identity to SEQ ID NO: 49. Accordingly, in one embodiment, the dcuC gene has at least about 90% identity to SEQ ID NO: 49. Accordingly, in one embodiment, the dcuC gene has at least about 95% identity to SEQ ID NO: 49. Accordingly, in one embodiment, the dcuC gene has at least about 85%, 86%, 87%, 88%, 89%, 90%, 91%, 92%, 93%, 94%, 95%, 96%, 97%, 98%, or 99% identity to SEQ ID NO: 49. In another embodiment, the dcuC gene comprises the sequence of SEQ ID NO: 49. In yet another embodiment the dcuC gene consists of the sequence of SEQ ID NO:70.

In one embodiment, the at least one propionate catabolism enzyme is DcuC. Accordingly, in one embodiment, DcuC has at least about 80% identity with SEQ ID NO: 129. In one embodiment, DcuC has at least about 90% identity with SEQ ID NO: 129. In another embodiment, DcuC has at least about 95% identity with SEQ ID NO: 129. Accordingly, in one embodiment, DcuC has at least about 85%, 86%, 89%, 88%, 89%, 90%, 91%, 92%, 93%, 94%, 95%, 96%, 97%, 98%, or 99% identity with SEQ ID NO: 129. In another embodiment, DcuC comprises a sequence which encodes SEQ ID NO: 129. In yet another embodiment, DcuC consists of a sequence which encodes SEQ ID NO: 129.

In one embodiment, the succinate exporter is DcuC. In one embodiment, the dcuC gene has at least about 80% identity to SEQ ID NO: 118. Accordingly, in one embodiment, the dcuC gene has at least about 90% identity to SEQ ID NO: 118. Accordingly, in one embodiment, the dcuC gene has at least about 95% identity to SEQ ID NO: 118. Accordingly, in one embodiment, the dcuC gene has at least about 85%, 86%, 87%, 88%, 89%, 90%, 91%, 92%, 93%, 94%, 95%, 96%, 97%, 98%, or 99% identity to SEQ ID NO: 118. In another embodiment, the dcuC gene comprises the sequence of SEQ ID NO: 118. In yet another embodiment the dcuC gene consists of the sequence of SEQ ID NO: 118.

In one embodiment, the at least one propionate catabolism enzyme is DcuC. Accordingly, in one embodiment, DcuC has at least about 80% identity with SEQ ID NO: 117. In one embodiment, DcuC has at least about 90% identity with SEQ ID NO: 117. In another embodiment, DcuC has at least about 95% identity with SEQ ID NO: 117. Accordingly, in one embodiment, DcuC has at least about 85%, 86%, 89%, 88%, 89%, 90%, 91%, 92%, 93%, 94%, 95%, 96%, 97%, 98%, or 99% identity with SEQ ID NO: 117. In another embodiment, DcuC comprises a sequence which encodes SEQ ID NO: 117. In yet another embodiment, DcuC consists of a sequence which encodes SEQ ID NO: 117.

In another embodiment, the succinate exporter is SucE1. In one embodiment, the sucE1 gene has at least about 80% identity to SEQ ID NO: 46. Accordingly, in one embodiment, the sucE1 gene has at least about 90% identity to SEQ ID NO: 46. Accordingly, in one embodiment, the sucE1 gene has at least about 95% identity to SEQ ID NO: 46. Accordingly, in one embodiment, the sucE1 gene has at least about 85%, 86%, 87%, 88%, 89%, 90%, 91%, 92%, 93%, 94%, 95%, 96%, 97%, 98%, or 99% identity to SEQ ID NO: 46. In another embodiment, the sucE1 gene comprises the sequence of SEQ ID NO: 46. In yet another embodiment the sucE1 gene consists of the sequence of SEQ ID NO: 46.

In another embodiment, the succinate exporter is SucE1. In one embodiment, the sucE1 gene has at least about 80% identity to SEQ ID NO: 120. Accordingly, in one embodiment, the sucE1 gene has at least about 90% identity to SEQ ID NO: 120. Accordingly, in one embodiment, the sucE1 gene has at least about 95% identity to SEQ ID NO: 120. Accordingly, in one embodiment, the sucE1 gene has at least about 85%, 86%, 87%, 88%, 89%, 90%, 91%, 92%, 93%, 94%, 95%, 96%, 97%, 98%, or 99% identity to SEQ ID NO: 120. In another embodiment, the sucE1 gene comprises the sequence of SEQ ID NO: 120. In yet another embodiment the sucE1 gene consists of the sequence of SEQ ID NO: 120.

In one embodiment, the at least one succinate exporter is sucE1. Accordingly, in one embodiment, sucE1 has at least about 80% identity with SEQ ID NO: 128. In one embodiment, sucE1 has at least about 90% identity with SEQ ID NO: 128. In another embodiment, sucE1 has at least about 95% identity with SEQ ID NO: 128. Accordingly, in one embodiment, sucE1 has at least about 85%, 86%, 89%, 88%, 89%, 90%, 91%, 92%, 93%, 94%, 95%, 96%, 97%, 98%, or 99% identity with SEQ ID NO: 128. In another embodiment, sucE1 comprises a sequence which encodes SEQ ID NO: 128. In yet another embodiment, sucE1 consists of a sequence which encodes SEQ ID NO: 128. In another embodiment, the sucE1 has at least about 80% identity with SEQ ID NO: 119. In one embodiment, sucE1 has at least about 90% identity with SEQ ID NO: 119. In another embodiment, sucE1 has at least about 95% identity with SEQ ID NO: 119. Accordingly, in one embodiment, sucE1 has at least about 85%, 86%, 89%, 88%, 89%, 90%, 91%, 92%, 93%, 94%, 95%, 96%, 97%, 98%, or 99% identity with SEQ ID NO: 119. In another embodiment, sucE1 comprises a sequence which encodes SEQ ID NO: 119. In yet another embodiment, sucE1 consists of a sequence which encodes SEQ ID NO: 119.

In some embodiments, the bacterial cell comprises at least one heterologous gene encoding at least one propionate catabolism enzyme operably linked to a first promoter and at least one heterologous gene encoding an exporter of succinate. In some embodiments, the at least one heterologous gene encoding an exporter of succinate is operably linked to the first promoter. In other embodiments, the at least one heterologous gene encoding an exporter of succinate is operably linked to a second promoter. In one embodiment, the at least one gene encoding an exporter of succinate is directly operably linked to the second promoter. In another embodiment, the at least one gene encoding an exporter of succinate is indirectly operably linked to the second promoter.

In some embodiments, expression of at least one gene encoding an exporter of succinate is controlled by a different promoter than the promoter that controls expression of the at least one gene encoding the at least one propionate catabolism enzyme. In some embodiments, expression of the at least one gene encoding an exporter of succinate is controlled by the same promoter that controls expression of the at least one propionate catabolism enzyme. In some embodiments, at least one gene encoding an exporter of succinate and the propionate catabolism enzyme are divergently transcribed from a promoter region. In some embodiments, expression of each of genes encoding the at least one gene encoding an exporter of succinate and the at least one gene encoding the at least one propionate catabolism enzyme is controlled by different promoters.

In one embodiment, the promoter is not operably linked with the at least one gene encoding an exporter of succinate in nature. In some embodiments, the at least one gene encoding the exporter of succinate is controlled by its native promoter. In some embodiments, the at least one gene encoding the exporter of succinate is controlled by an inducible promoter. In some embodiments, the at least one gene encoding the exporter of succinate is controlled by a promoter that is stronger than its native promoter. In some embodiments, the at least one gene encoding the exporter of succinate is controlled by a constitutive promoter.

In another embodiment, the promoter is an inducible promoter. Inducible promoters are described in more detail infra.

In one embodiment, the at least one gene encoding an exporter of succinate is located on a plasmid in the bacterial cell. In another embodiment, the at least one gene encoding an exporter of succinate is located in the chromosome of the bacterial cell. In yet another embodiment, a native copy of the at least one gene encoding an exporter of succinate is located in the chromosome of the bacterial cell, and a copy of at least one gene encoding an exporter of succinate from a different species of bacteria is located on a plasmid in the bacterial cell. In yet another embodiment, a native copy of the at least one gene encoding an exporter of a succinate is located on a plasmid in the bacterial cell, and a copy of at least one gene encoding an exporter of succinate from a different species of bacteria is located on a plasmid in the bacterial cell. In yet another embodiment, a native copy of the at least one gene encoding an exporter of succinate is located in the chromosome of the bacterial cell, and a copy of the at least one gene encoding an exporter of succinate from a different species of bacteria is located in the chromosome of the bacterial cell.

In some embodiments, the at least one native gene encoding the exporter in the bacterial cell is not modified, and one or more additional copies of the native exporter are inserted into the genome. In one embodiment, the one or more additional copies of the native exporter that is inserted into the genome are under the control of the same inducible promoter that controls expression of the at least one gene encoding the propionate catabolism enzyme, e.g., the FNR responsive promoter, or a different inducible promoter than the one that controls expression of the at least one propionate catabolism enzyme, or a constitutive promoter. In alternate embodiments, the at least one native gene encoding the exporter is not modified, and one or more additional copies of the exporter from a different bacterial species is inserted into the genome of the bacterial cell. In one embodiment, the one or more additional copies of the exporter inserted into the genome of the bacterial cell are under the control of the same inducible promoter that controls expression of the at least one gene encoding the propionate catabolism enzyme, e.g., the FNR responsive promoter, or a different inducible promoter than the one that controls expression of the at least one gene encoding the at least one propionate catabolism enzyme, or a constitutive promoter.

In one embodiment, when the exporter of succinate is expressed in the engineered bacterial cells, the bacterial cells export 10% more succinate out of the bacterial cell when the exporter is expressed than unmodified bacteria of the same bacterial subtype under the same conditions. In another embodiment, when the exporter of succinate is expressed in the engineered bacterial cells, the bacterial cells export 20%, 30%, 40%, 50%, 60%, 70%, 80%, 90% or 100% more succinate out of the bacterial cell when the exporter is expressed than unmodified bacteria of the same bacterial subtype under the same conditions. In yet another embodiment, when the exporter of succinate is expressed in the engineered bacterial cells, the bacterial cells export two-fold more succinate out of the cell when the exporter is expressed than unmodified bacteria of the same bacterial subtype under the same conditions. In yet another embodiment, when the exporter of succinate is expressed in the engineered bacterial cells, the bacterial cells export three-fold, four-fold, five-fold, six-fold, seven-fold, eight-fold, nine-fold, or ten-fold more succinate out of the cell when the exporter is expressed than unmodified bacteria of the same bacterial subtype under the same conditions.

Nucleic Acids

In some embodiments, the disclosure provides novel nucleic acids for metabolizing propionic acid. In some embodiments, the nucleic acid comprises gene sequence encoding one or more molecules that metabolize propionic acid. In some embodiments, the nucleic acid comprises gene sequence encoding PrpE. In some embodiments, the nucleic acid comprises gene sequence encoding PhaA. In some embodiments, the nucleic acid comprises gene sequence encoding PhaB. In some embodiments, the nucleic acid comprises gene sequence encoding PhaC. In some embodiments, the nucleic acid comprises gene sequence encoding PrpE and PhaA. In some embodiments, the nucleic acid comprises gene sequence encoding PrpE and PhaB. In some embodiments, the nucleic acid comprises gene sequence encoding PrpE and PhaC. In some embodiments, the nucleic acid comprises gene sequence encoding PhaA and PhaB. In some embodiments, the nucleic acid comprises gene sequence encoding PhaA and PhaC. In some embodiments, the nucleic acid comprises gene sequence encoding PhaB and PhaC. In some embodiments, the nucleic acid comprises gene sequence encoding PrpE, PhaA, and PhaB. In some embodiments, the nucleic acid comprises gene sequence encoding PrpE, PhaA, and PhaC. In some embodiments, the nucleic acid comprises gene sequence encoding PrpE, PhaB, and PhaC. In some embodiments, the nucleic acid comprises gene sequence encoding PhaA, PhaB, and PhaC. In some embodiments, the nucleic acid comprises gene sequence encoding PrpE, PhaA, PhaB, and PhaC.

In some embodiments, the disclosure provides novel nucleic acids for transporting propionate. In some embodiments, the nucleic acid comprises gene sequence encoding one or more molecules that transport propionic acid. In some embodiments, the disclosure provides novel nucleic acids for exporting succinate. In some embodiments, the nucleic acid comprises gene sequence encoding one or more molecules that export succinate. In some embodiments, the nucleic acid encoding PrpE and/or PhaA and/or PhaB and/or PhaC further comprises gene sequence encoding propionate transporter, e.g., mctC and/or PutB_6/. In some embodiments, the nucleic acid encoding PrpE and/or PhaA and/or PhaB and/or PhaC further comprises gene sequence encoding a succinate transporter DeuC. In some embodiments, the nucleic acid encoding PrpE and/or PhaA and/or PhaB and/or PhaC further comprises gene sequence encoding succinate exporter sucE1.

In some embodiments, the disclosure provides novel nucleic acids for metabolizing propionic acid. In some embodiments, the nucleic acid comprises gene sequence encoding one or more molecules that metabolize propionic acid. In some embodiments, the nucleic acid comprises gene sequence encoding PrpE. In some embodiments, the nucleic acid comprises gene sequence encoding accA. In some embodiments, the nucleic acid comprises gene sequence encoding pccB. In some embodiments, the nucleic acid comprises gene sequence encoding mmcE. In some embodiments, the nucleic acid comprises gene sequence encoding mutA. In some embodiments, the nucleic acid comprises gene sequence encoding mutB. In some embodiments, the nucleic acid comprises gene sequence encoding PrpE and accA. In some embodiments, the nucleic acid comprises gene sequence encoding PrpE and pccB. In some embodiments, the nucleic acid comprises gene sequence encoding PrpE and mmcE. In some embodiments, the nucleic acid comprises gene sequence encoding PrpE and mutA. In some embodiments, the nucleic acid comprises gene sequence encoding PrpE and mutB. In some embodiments, the nucleic acid comprises gene sequence encoding accA and pccB. In some embodiments, the nucleic acid comprises gene sequence encoding accA and mmcE. In some embodiments, the nucleic acid comprises gene sequence encoding accA and mutA. In some embodiments, the nucleic acid comprises gene sequence encoding accA and mutB. In some embodiments, the nucleic acid comprises gene sequence encoding pccB and mmcE. In some embodiments, the nucleic acid comprises gene sequence encoding pccB and mutA. In some embodiments, the nucleic acid comprises gene sequence encoding pccB and mutB. In some embodiments, the nucleic acid comprises gene sequence encoding mmcE and mutA. In some embodiments, the nucleic acid comprises gene sequence encoding mmcE and mutB. In some embodiments, the nucleic acid comprises gene sequence encoding mutA and mutB. In some embodiments, the nucleic acid comprises gene sequence encoding PrpE, accA, and pccB. In some embodiments, the nucleic acid comprises gene sequence encoding PrpE, accA, and mmcE. In some embodiments, the nucleic acid comprises gene sequence encoding PrpE, accA, and mutA. In some embodiments, the nucleic acid comprises gene sequence encoding PrpE, accA, and mutB. In some embodiments, the nucleic acid comprises gene sequence encoding PrpE, pccB, and mmcE. In some embodiments, the nucleic acid comprises gene sequence encoding PrpE, pccB and mutA. In some embodiments, the nucleic acid comprises gene sequence encoding PrpE, pccB and mutB. In some embodiments, the nucleic acid comprises gene sequence encoding PrpE, mmcE, and mutA. In some embodiments, the nucleic acid comprises gene sequence encoding PrpE, mmcE, and mutB. In some embodiments, the nucleic acid comprises gene sequence encoding PrpE, mutA, and mutB. In some embodiments, the nucleic acid comprises gene sequence encoding accA, pccB, and mmcE. In some embodiments, the nucleic acid comprises gene sequence encoding accA, pccB, and mutA. In some embodiments, the nucleic acid comprises gene sequence encoding accA, pccB, and mutB. In some embodiments, the nucleic acid comprises gene sequence encoding accA, mmcE, and mutA. In some embodiments, the nucleic acid comprises gene sequence encoding accA, mmcE, and mutB. In some embodiments, the nucleic acid comprises gene sequence encoding accA, mutA, and mutB. In some embodiments, the nucleic acid comprises gene sequence encoding pccB, mmcE, and mutA. In some embodiments, the nucleic acid comprises gene sequence encoding pccB, mmcE, and mutB. In some embodiments, the nucleic acid comprises gene sequence encoding mmcE, mutA, and mutB. In some embodiments, the nucleic acid comprises gene sequence encoding PrpE, accA, pccB, and mmcE. In some embodiments, the nucleic acid comprises gene sequence encoding PrpE, accA, pccB and mutA. In some embodiments, the nucleic acid comprises gene sequence encoding PrpE, accA, pccB, and mutB. In some embodiments, the nucleic acid comprises gene sequence encoding PrpE, accA, mmcE, and mutA. In some embodiments, the nucleic acid comprises gene sequence encoding PrpE, accA, mmcE and mutB. In some embodiments, the nucleic acid comprises gene sequence encoding PrpE, accA, mutA, and mutB. In some embodiments, the nucleic acid comprises gene sequence encoding PrpE, pccB, mmcE, and mutA. In some embodiments, the nucleic acid comprises gene sequence encoding PrpE, pccB, mmcE, and mutB. In some embodiments, the nucleic acid comprises gene sequence encoding PrpE, pccB, mutA, and mutB. In some embodiments, the nucleic acid comprises gene sequence encoding PrpE, mmcE, mutA, and mutB. In some embodiments, the nucleic acid comprises gene sequence encoding accA, pccB, mmcE, and mutA. In some embodiments, the nucleic acid comprises gene sequence encoding accA, pccB, mmcE, and mutB. In some embodiments, the nucleic acid comprises gene sequence encoding accA, pccB, mutA, and mutB. In some embodiments, the nucleic acid comprises gene sequence encoding accA, mmcE, mutA, and mutB. In some embodiments, the nucleic acid comprises gene sequence encoding pccB, mmcE, mutA, and mutB. In some embodiments, the nucleic acid comprises gene sequence encoding PrpE, accA, pccB, mmcE, mutA, and mutB.

In some embodiments, the disclosure provides novel nucleic acids for transporting propionate. In some embodiments, the nucleic acid comprises gene sequence encoding one or more molecules that transport propionic acid. In some embodiments, the disclosure provides novel nucleic acids for exporting succinate. In some embodiments, the nucleic acid comprises gene sequence encoding one or more molecules that export succinate. In some embodiments, the nucleic acid encoding PrpE and/or accA and/or pccB and/or mmcE and/or mutA and/or mutB further comprises gene sequence encoding propionate transporter, e.g., mctC and/or PutB_6/. In some embodiments, the nucleic acid encoding PrpE and/or accA and/or pccB and/or mmcE and/or mutA and/or mutB further comprises gene sequence encoding a succinate transporter DeuC. In some embodiments, the nucleic acid encoding PrpE and/or accA and/or pccB and/or mmcE and/or mutA and/or mutB further comprises gene sequence encoding succinate exporter sucE1.

In some embodiments, the disclosure provides novel nucleic acids for metabolizing propionic acid. In some embodiments, the nucleic acid comprises gene sequence encoding one or more molecules that metabolize propionic acid. In some embodiments, the nucleic acid comprises gene sequence encoding PrpE. In some embodiments, the nucleic acid comprises gene sequence encoding PrpB. In some embodiments, the nucleic acid comprises gene sequence encoding PrpC In some embodiments, the nucleic acid comprises gene sequence encoding PrpD. In some embodiments, the nucleic acid comprises gene sequence encoding PrpE and PrpB. In some embodiments, the nucleic acid comprises gene sequence encoding PrpE and PrpC. In some embodiments, the nucleic acid comprises gene sequence encoding PrpE and PrpD. In some embodiments, the nucleic acid comprises gene sequence encoding PrpB and PrpC. In some embodiments, the nucleic acid comprises gene sequence encoding PrpB and PrpD. In some embodiments, the nucleic acid comprises gene sequence encoding PrpC and PrpD. In some embodiments, the nucleic acid comprises gene sequence encoding PrpE, PrpB, and PrpC. In some embodiments, the nucleic acid comprises gene sequence encoding PrpE, PrpB and PrpD. In some embodiments, the nucleic acid comprises gene sequence encoding PrpE, PrpC, and PrpD. In some embodiments, the nucleic acid comprises gene sequence encoding PrpB, PrpC, and PrpD. In some embodiments, the nucleic acid comprises gene sequence encoding PrpE, PrpB, PrpC, and PrpD.

In some embodiments, the disclosure provides novel nucleic acids for transporting propionate. In some embodiments, the nucleic acid comprises gene sequence encoding one or more molecules that transport propionic acid. In some embodiments, the disclosure provides novel nucleic acids for exporting succinate. In some embodiments, the nucleic acid comprises gene sequence encoding one or more molecules that export succinate. In some embodiments, the nucleic acid encoding PrpE and/or PrpD and/or PrpC and/or PrpB further comprises gene sequence encoding, propionate transporter, e.g., mctC and/or PutB_6/. In some embodiments, the nucleic acid encoding PrpE and/or PrpD and/or PrpC and/or PrpB further comprises gene sequence encoding a succinate transporter DeuC. In some embodiments, the nucleic acid encoding PrpE and/or PrpD and/or PrpC and/or PrpB further comprises gene sequence encoding succinate exporter sucE1.

In some embodiments, the nucleic acid comprises gene sequence encoding PHA pathway cassette, comprising PrpE, PhaA, PhaB, and PhaC. In some embodiments, the nucleic acid comprises gene sequence encoding MMCA pathway cassette comprising PrpE, accA, pccB, mmcE, mutA, and mutB. In some embodiments, the nucleic acid comprises gene sequence encoding M2C cassette comprising PrpE, PrpB, PrpC, and PrpD. In some embodiments, the nucleic acid comprises gene sequence encoding PHA pathway cassette and MMCA pathway cassette. In some embodiments, the nucleic acid comprises gene sequence encoding PHA pathway cassette and M2C pathway cassette. In some embodiments, the nucleic acid comprises gene sequence encoding MMCA pathway cassette and M2C pathway cassette. In some embodiments, the nucleic acid comprises gene sequence encoding PHA pathway cassette, MMCA pathway cassette and a M2C cassette.

In some embodiments, the nucleic acid encoding one or more propionate catabolism cassettes, selected from PHA pathway cassette, MMCA pathway cassette and a M2C cassette further comprises gene sequence encoding propionate transporter, e.g., mctC and/or PutB_6/. In some embodiments, the nucleic acid encoding one or more propionate catabolism cassettes, selected from PHA pathway cassette, MMCA pathway cassette and a M2C cassette further comprises gene sequence encoding a succinate transporter DcuC. In some embodiments, the nucleic acid encoding one or more propionate catabolism cassettes, selected from PHA pathway cassette, MMCA pathway cassette and a M2C cassette further comprises gene sequence encoding succinate exporter sucE1.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises prpE (encoding propionate-CoA ligase PrpE). Accordingly, in one embodiment, the nucleic acid sequence comprising the prpE gene has at least about 80% identity with SEQ ID NO: 25. In another embodiment, the nucleic acid sequence comprising the prpE gene has at least about 80% identity with SEQ ID NO: 73. Accordingly, in one embodiment, the nucleic acid sequence comprising the prpE gene has at least about 90% identity with SEQ ID NO: 25. In another embodiment, the nucleic acid sequence comprising the prpE gene has at least about 90% identity with SEQ ID NO: 73. Accordingly, in one embodiment, the nucleic acid sequence comprising the prpE gene has at least about 95% identity with SEQ ID NO: 25. In another embodiment, the nucleic acid sequence comprising the prpE gene has at least about 95% identity with SEQ ID NO: 73. Accordingly, in one embodiment, the nucleic acid sequence comprising the prpE gene has at least about 85%, 86%, 87%, 88%, 89%, 90%, 91%, 92%, 93%, 94%, 95%, 96%, 97%, 98%, or 99% identity with SEQ ID NO: 25. In another embodiment, the nucleic acid sequence comprising the prpE gene has at least about 85%, 86%, 87%, 88%, 89%, 90%, 91%, 92%, 93%, 94%, 95%, 96%, 97%, 98%, or 99% identity with SEQ ID NO: 73. In another embodiment, the nucleic acid sequence comprising the prpE gene comprises the sequence of SEQ ID NO: 25. In another embodiment, the nucleic acid sequence comprising the prpE gene comprises the sequence of SEQ ID NO: 73. In yet another embodiment the nucleic acid sequence comprising the prpE gene consists of the sequence of SEQ ID NO: 25. In another embodiment, the nucleic acid sequence comprising the prpE gene consists of the sequence of SEQ ID NO: 73.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises prpC (encoding PrpC, a 2-methylcitrate synthetase). Accordingly, in one embodiment, the nucleic acid sequence comprising the prpC gene has at least about 80% identity with SEQ ID NO: 57. In another embodiment, the nucleic acid sequence comprising the prpC gene has at least about 80% identity with SEQ ID NO:76. Accordingly, in one embodiment, the nucleic acid sequence comprising the prpC gene has at least about 90% identity with SEQ ID NO: 57. In another embodiment, the nucleic acid sequence comprising the prpC gene has at least about 90% identity with SEQ ID NO: 76. Accordingly, in one embodiment, the nucleic acid sequence comprising the prpC gene has at least about 95% identity with SEQ ID NO: 57. In another embodiment, the nucleic acid sequence comprising the prpC gene has at least about 95% identity with SEQ ID NO: 76. Accordingly, in one embodiment, the nucleic acid sequence comprising the prpC gene has at least about 85%, 86%, 87%, 88%, 89%, 90%, 91%, 92%, 93%, 94%, 95%, 96%, 97%, 98%, or 99% identity with SEQ ID NO: 57. In another embodiment, the nucleic acid sequence comprising the prpC gene has at least about 85%, 86%, 87%, 88%, 89%, 90%, 91%, 92%, 93%, 94%, 95%, 96%, 97%, 98%, or 99% identity with SEQ ID NO: 76. In another embodiment, the nucleic acid sequence comprising the prpC gene comprises the sequence of SEQ ID NO: 57. In another embodiment, the nucleic acid sequence comprising the prpC gene comprises the sequence of SEQ ID NO: 76. In yet another embodiment the nucleic acid sequence comprising the prpC gene consists of the sequence of SEQ ID NO: 57. In another embodiment, the nucleic acid sequence comprising the prpC gene consists of the sequence of SEQ ID NO: 76.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises prpD (encoding PrpD, a 2-methylcitrate dehydrogenase). Accordingly, in one embodiment, the nucleic acid sequence comprising the prpD gene has at least about 80% identity with SEQ ID NO: 58. In another embodiment, the nucleic acid sequence comprising the prpD gene has at least about 80% identity with SEQ ID NO: 79. Accordingly, in one embodiment, the nucleic acid sequence comprising the prpD gene has at least about 90% identity with SEQ ID NO: 58. In another embodiment, the nucleic acid sequence comprising the prpD gene has at least about 90% identity with SEQ ID NO: 79. Accordingly, in one embodiment, the nucleic acid sequence comprising the prpD gene has at least about 95% identity with SEQ ID NO: 58. In another embodiment, the nucleic acid sequence comprising the prpD gene has at least about 95% identity with SEQ ID NO: 79. Accordingly, in one embodiment, the nucleic acid sequence comprising the prpD gene has at least about 85%, 86%, 87%, 88%, 89%, 90%, 91%, 92%, 93%, 94%, 95%, 96%, 97%, 98%, or 99% identity with SEQ ID NO: 58. In another embodiment, the nucleic acid sequence comprising the prpD gene has at least about 85%, 86%, 87%, 88%, 89%, 90%, 91%, 92%, 93%, 94%, 95%, 96%, 97%, 98%, or 99% identity with SEQ ID NO: 79. In another embodiment, the nucleic acid sequence comprising the prpD gene comprises the sequence of SEQ ID NO: 58. In another embodiment, the nucleic acid sequence comprising the prpD gene comprises the sequence of SEQ ID NO: 79. In yet another embodiment the nucleic acid sequence comprising the prpD gene consists of the sequence of SEQ ID NO: 58. In another embodiment, the nucleic acid sequence comprising the prpD gene consists of the sequence of SEQ ID NO: 79.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises prpB (encoding PrpB, a 2-methylisocitrate lyase). Accordingly, in one embodiment, the nucleic acid sequence comprising the prpB gene has at least about 80% identity with SEQ ID NO: 56. In another embodiment, the nucleic acid sequence comprising the prpB gene has at least about 80% identity with SEQ ID NO: 82. Accordingly, in one embodiment, the nucleic acid sequence comprising the prpB gene has at least about 90% identity with SEQ ID NO: 56. In another embodiment, the nucleic acid sequence comprising the prpB gene has at least about 90% identity with SEQ ID NO: 82. Accordingly, in one embodiment, the nucleic acid sequence comprising the prpB gene has at least about 95% identity with SEQ ID NO: 56. In another embodiment, the nucleic acid sequence comprising the prpB gene has at least about 95% identity with SEQ ID NO: 82. Accordingly, in one embodiment, the nucleic acid sequence comprising the prpB gene has at least about 85%, 86%, 87%, 88%, 89%, 90%, 91%, 92%, 93%, 94%, 95%, 96%, 97%, 98%, or 99% identity with SEQ ID NO: 56. In another embodiment, the nucleic acid sequence comprising the prpB gene has at least about 85%, 86%, 87%, 88%, 89%, 90%, 91%, 92%, 93%, 94%, 95%, 96%, 97%, 98%, or 99% identity with SEQ ID NO: 82. In another embodiment, the nucleic acid sequence comprising the prpB gene comprises the sequence of SEQ ID NO: 56. In another embodiment, the nucleic acid sequence comprising the prpB gene comprises the sequence of SEQ ID NO: 82. In yet another embodiment the nucleic acid sequence comprising the prpB gene consists of the sequence of SEQ ID NO: 56. In another embodiment, the nucleic acid sequence comprising the prpB gene consists of the sequence of SEQ ID NO: 82.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises phaB (encoding PhaB, a acetoacetyl-CoA reductase). Accordingly, in one embodiment, the nucleic acid sequence comprising the phaB gene has at least about 80% identity with SEQ ID NO: 26. In one embodiment, the nucleic acid sequence comprising the phaB gene has at least about 90% identity with SEQ ID NO: 26. In another embodiment, the nucleic acid sequence comprising the phaB gene has at least about 95% identity with SEQ ID NO: 26. Accordingly, in one embodiment, the nucleic acid sequence comprising the phaB gene has at least about 85%, 86%, 87%, 88%, 89%, 90%, 91%, 92%, 93%, 94%, 95%, 96%, 97%, 98%, or 99% identity with SEQ ID NO: 26. In another embodiment, the nucleic acid sequence comprising the phaB gene comprises SEQ ID NO: 26. In yet another embodiment the nucleic acid sequence comprising the phaB gene consists of SEQ ID NO: 26.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises phaC (encoding PhaC, a polyhydroxyalkanoate synthase). Accordingly, in one embodiment, the nucleic acid sequence comprising the phaC gene has at least about 80% identity SEQ ID NO: 27. In one embodiment, the nucleic acid sequence comprising the phaC gene has at least about 90% identity with SEQ ID NO: 27. In another embodiment, the nucleic acid sequence comprising the phaC gene has at least about 95% identity with SEQ ID NO: 27. Accordingly, in one embodiment, the nucleic acid sequence comprising the phaC gene has at least about 85%, 86%, 87%, 88%, 89%, 90%, 91%, 92%, 93%, 94%, 95%, 96%, 97%, 98%, or 99% identity with SEQ ID NO: 27. In another embodiment, the nucleic acid sequence comprising the phaC gene comprises SEQ ID NO: 27. In yet another embodiment the nucleic acid sequence comprising the phaC gene consists of SEQ ID NO: 27.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises phaA (encoding PhaA, a beta-ketothiolase). Accordingly, in one embodiment, the nucleic acid sequence comprising the phaA gene has at least about 80% identity with a sequence which encodes SEQ ID NO: 28. In one embodiment, the nucleic acid sequence comprising the phaA gene has at least about 90% identity with a sequence which encodes SEQ ID NO: 28. In another embodiment, the nucleic acid sequence comprising the phaA gene has at least about 95% identity with a sequence which encodes SEQ ID NO: 28. Accordingly, in one embodiment, the nucleic acid sequence comprising the phaA gene has at least about 85%, 86%, 87%, 88%, 89%, 90%, 91%, 92%, 93%, 94%, 95%, 96%, 97%, 98%, or 99% identity with a sequence which encodes SEQ ID NO: 28. In another embodiment, the nucleic acid sequence comprising the phaA gene comprises a sequence which encodes SEQ ID NO: 28. In yet another embodiment the nucleic acid sequence comprising the phaA gene consists of a sequence which encodes SEQ ID NO: 28.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises pccB (encoding PccB, a propionyl CoA carboxylase). Accordingly, in one embodiment, the nucleic acid sequence comprising the pccB gene has at least about 80% identity with SEQ ID NO: 39. In one embodiment, the nucleic acid sequence comprising the pccB gene has at least about 90% identity with SEQ ID NO: 39. In one embodiment, the nucleic acid sequence comprising the pccB gene has at least about 95% identity with SEQ ID NO: 39. In one embodiment, the nucleic acid sequence comprising the pccB gene has at least about 85%, 86%, 87%, 88%, 89%, 90%, 91%, 92%, 93%, 94%, 95%, 96%, 97%, 98%, or 99% identity with SEQ ID NO: 39. In another embodiment, the nucleic acid sequence comprising the pccB gene comprises the sequence of SEQ ID NO: 39. In yet another embodiment, the nucleic acid sequence comprising the pccB gene consists of the sequence of SEQ ID NO: 39.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises pccB. Accordingly, in one embodiment, the nucleic acid sequence comprising the pccB gene has at least about 80% identity with SEQ ID NO: 96. In one embodiment, the nucleic acid sequence comprising the pccB gene has at least about 90% identity with SEQ ID NO: 96. In one embodiment, the nucleic acid sequence comprising the pccB gene has at least about 95% identity with SEQ ID NO: 96. In one embodiment, the nucleic acid sequence comprising the pccB gene has at least about 85%, 86%, 87%, 88%, 89%, 90%, 91%, 92%, 93%, 94%, 95%, 96%, 97%, 98%, or 99% identity with SEQ ID NO: 96. In another embodiment, the nucleic acid sequence comprising the pccB gene comprises the sequence of SEQ ID NO: 96. In yet another embodiment, the nucleic acid sequence comprising the pccB gene consists of the sequence of SEQ ID NO: 96.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises accA1 (encoding AccA1, an acetyl CoA carboxylase). Accordingly, in one embodiment, the nucleic acid sequence comprising the accA1 gene has at least about 80% identity with SEQ ID NO: 38. In one embodiment, the nucleic acid sequence comprising the accA1 gene has at least about 90% identity with SEQ ID NO: 38. In one embodiment, the nucleic acid sequence comprising the accA1 gene has at least about 95% identity with SEQ ID NO: 38. In one embodiment, the nucleic acid sequence comprising the accA1 gene has at least about 85%, 86%, 87%, 88%, 89%, 90%, 91%, 92%, 93%, 94%, 95%, 96%, 97%, 98%, or 99% identity with SEQ ID NO: 38. In another embodiment, the nucleic acid sequence comprising the accA1 gene comprises the sequence of SEQ ID NO: 38. In yet another embodiment, the nucleic acid sequence comprising the accA1 gene consists of the sequence of SEQ ID NO: 38.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme encodes accA (encoding AccA1, an acetyl CoA carboxylase). Accordingly, in one embodiment, the nucleic acid sequence comprising the accA1 gene has at least about 80% identity with SEQ ID NO: 104. In one embodiment, the nucleic acid sequence comprising the accA1 gene has at least about 90% identity with SEQ ID NO: 104. In one embodiment, the nucleic acid sequence comprising the accA1 gene has at least about 95% identity with SEQ ID NO: 104. In one embodiment, the nucleic acid sequence comprising the accA1 gene has at least about 85%, 86%, 87%, 88%, 89%, 90%, 91%, 92%, 93%, 94%, 95%, 96%, 97%, 98%, or 99% identity with SEQ ID NO: 104. In another embodiment, the nucleic acid sequence comprising the accA1 gene comprises the sequence of SEQ ID NO: 104. In yet another embodiment, the nucleic acid sequence comprising the accA1 gene consists of the sequence of SEQ ID NO: 104.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises mmcE (encoding MmcE, a methylmalonyl-CoA mutase). Accordingly, in one embodiment, the nucleic acid sequence comprising the mmcE gene has at least about 80% identity with SEQ ID NO: 32. In one embodiment, the nucleic acid sequence comprising the mmcE gene has at least about 90% identity with SEQ ID NO: 32. In one embodiment, the nucleic acid sequence comprising the mmcE gene has at least about 95% identity with SEQ ID NO: 32. In one embodiment, the nucleic acid sequence comprising the mmcE gene has at least about 85%, 86%, 87%, 88%, 89%, 90%, 91%, 92%, 93%, 94%, 95%, 96%, 97%, 98%, or 99% identity with SEQ ID NO: 32. In another embodiment, the nucleic acid sequence comprising the mmcE gene comprises the sequence of SEQ ID NO: 32. In yet another embodiment, the nucleic acid sequence comprising the mmcE gene consists of the sequence of SEQ ID NO: 32.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises mmcE. Accordingly, in one embodiment, the nucleic acid sequence comprising the mmcE gene has at least about 80% identity with SEQ ID NO: 106. In one embodiment, the nucleic acid sequence comprising the mmcE gene has at least about 90% identity with SEQ ID NO: 106. In one embodiment, the nucleic acid sequence comprising the mmcE gene has at least about 95% identity with SEQ ID NO: 106. In one embodiment, the nucleic acid sequence comprising the mmcE gene has at least about 85%, 86%, 87%, 88%, 89%, 90%, 91%, 92%, 93%, 94%, 95%, 96%, 97%, 98%, or 99% identity with SEQ ID NO: 106. In another embodiment, the nucleic acid sequence comprising the mmcE gene comprises the sequence of SEQ ID NO: 106. In yet another embodiment, the nucleic acid sequence comprising the mmcE gene consists of the sequence of SEQ ID NO: 106.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises mutA (encodes MutA, a methylmalonyl-CoA mutase small subunit). Accordingly, in one embodiment, the nucleic acid sequence comprising the mutA gene has at least about 80% identity with SEQ ID NO: 33. In one embodiment, the nucleic acid sequence comprising the mutA gene has at least about 90% identity with SEQ ID NO: 33. In one embodiment, the nucleic acid sequence comprising the mutA gene has at least about 95% identity with SEQ ID NO: 33. In one embodiment, the nucleic acid sequence comprising the mutA gene has at least about 85%, 86%, 87%, 88%, 89%, 90%, 91%, 92%, 93%, 94%, 95%, 96%, 97%, 98%, or 99% identity with SEQ ID NO: 33. In another embodiment, the nucleic acid sequence comprising the mutA gene comprises the sequence of SEQ ID NO: 33. In yet another embodiment, the nucleic acid sequence comprising the mutA gene consists of the sequence of SEQ ID NO: 33.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises mutA. Accordingly, in one embodiment, the nucleic acid sequence comprising the mutA gene has at least about 80% identity with SEQ ID NO: 110. In one embodiment, the nucleic acid sequence comprising the mutA gene has at least about 90% identity with SEQ ID NO: 110. In one embodiment, the nucleic acid sequence comprising the mutA gene has at least about 95% identity with SEQ ID NO: 110. In one embodiment, the nucleic acid sequence comprising the mutA gene has at least about 85%, 86%, 87%, 88%, 89%, 90%, 91%, 92%, 93%, 94%, 95%, 96%, 97%, 98%, or 99% identity with SEQ ID NO: 110. In another embodiment, the nucleic acid sequence comprising the mutA gene comprises the sequence of SEQ ID NO: 110. In yet another embodiment, the nucleic acid sequence comprising the mutA gene consists of the sequence of SEQ ID NO: 110.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises mutB (encoding MutB, a methylmalonyl-CoA mutase large subunit). Accordingly, in one embodiment, the nucleic acid sequence comprising the mutB gene has at least about 80% identity with SEQ ID NO: 34. In one embodiment, the nucleic acid sequence comprising the mutB gene has at least about 90% identity with SEQ ID NO: 34. In one embodiment, the nucleic acid sequence comprising the mutB gene has at least about 95% identity with SEQ ID NO: 34. In one embodiment, the nucleic acid sequence comprising the mutB gene has at least about 85%, 86%, 87%, 88%, 89%, 90%, 91%, 92%, 93%, 94%, 95%, 96%, 97%, 98%, or 99% identity with SEQ ID NO: 34. In another embodiment, the nucleic acid sequence comprising the mutB gene comprises the sequence of SEQ ID NO: 34. In yet another embodiment, the nucleic acid sequence comprising the mutB gene consists of the sequence of SEQ ID NO: 34.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises mutB (encoding MutB, a methylmalonyl-CoA mutase large subunit). Accordingly, in one embodiment, the nucleic acid sequence comprising the mutB gene has at least about 80% identity with SEQ ID NO: 112. In one embodiment, the nucleic acid sequence comprising the mutB gene has at least about 90% identity with SEQ ID NO: 112. In one embodiment, the nucleic acid sequence comprising the mutB gene has at least about 95% identity with SEQ ID NO: 112. In one embodiment, the nucleic acid sequence comprising the mutB gene has at least about 85%, 86%, 87%, 88%, 89%, 90%, 91%, 92%, 93%, 94%, 95%, 96%, 97%, 98%, or 99% identity with SEQ ID NO: 112. In another embodiment, the nucleic acid sequence comprising the mutB gene comprises the sequence of SEQ ID NO: 112. In yet another embodiment, the nucleic acid sequence comprising the mutB gene consists of the sequence of SEQ ID NO: 112.

In one of the nucleic acid embodiments described herein, the propionate catabolism enzyme comprises prpE. In one embodiment, the nucleic acid sequence encodes a polypeptide, which has at least about 80% identity with SEQ ID NO: 71. In one embodiment, the nucleic acid sequence encodes a polypeptide, which has at least about 90% identity with SEQ ID NO: 71. In another embodiment, the nucleic acid sequence encodes a polypeptide, which has at least about 95% identity with SEQ ID NO: 71. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 71. In another embodiment, the nucleic acid sequence encodes a polypeptide, which comprises a sequence which encodes SEQ ID NO: 71. In yet another embodiment, the nucleic acid sequence encodes a polypeptide, which consists of a sequence which encodes SEQ ID NO: 71.

In one of the nucleic acid embodiments described herein, the propionate catabolism enzyme comprises PhaA. In one embodiment, the nucleic acid sequence encodes a polypeptide, which has at least about 80% identity with SEQ ID NO: 137. In one embodiment, the nucleic acid sequence encodes a polypeptide, which has at least about 90% identity with SEQ ID NO: 175. In another embodiment, the nucleic acid sequence encodes a polypeptide, which has at least about 95% identity with SEQ ID NO: 137. Accordingly, in one embodiment, phaA has at least about 85%, 86%, 87%, 88%, 89%, 90%, 91%, 92%, 93%, 94%, 95%, 96%, 97%, 98%, or 99% identity with SEQ ID NO: 137. In another embodiment, phaA comprises a sequence which encodes SEQ ID NO: 137. In yet another embodiment phaA consists of a sequence which encodes SEQ ID NO: 137.

In one of the nucleic acid embodiments described herein, the propionate catabolism enzyme comprises PhaB. In one embodiment, the nucleic acid sequence encodes a polypeptide, which has at least about 80% identity with SEQ ID NO: 135. In one embodiment, the nucleic acid sequence encodes a polypeptide, which has at least about 90% identity with SEQ ID NO: 135. In another embodiment, the nucleic acid sequence encodes a polypeptide, which has at least about 95% identity with SEQ ID NO: 135. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 135. In another embodiment, the nucleic acid sequence encodes a polypeptide, which comprises a sequence which encodes SEQ ID NO: 135. In yet another embodiment the nucleic acid sequence encodes a polypeptide, which consists of a sequence which encodes SEQ ID NO: 135.

In one of the nucleic acid embodiments described herein, the propionate catabolism enzyme comprises PhaC. In one embodiment, the nucleic acid sequence encodes a polypeptide, which has at least about 80% identity with SEQ ID NO: 136. In one embodiment, the nucleic acid sequence encodes a polypeptide, which has at least about 90% identity with SEQ ID NO: 136. In another embodiment, the nucleic acid sequence encodes a polypeptide, which has at least about 95% identity with SEQ ID NO: 136. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 136. In another embodiment, the nucleic acid sequence encodes a polypeptide, which comprises a sequence which encodes SEQ ID NO: 136. In yet another embodiment the nucleic acid sequence encodes a polypeptide, which consists of a sequence which encodes SEQ ID NO: 136.

In one of the nucleic acid embodiments described herein, the propionate catabolism enzyme comprises MmcE. In one embodiment, the nucleic acid sequence encodes a polypeptide, which has at least about 80% identity with SEQ ID NO: 132. In one embodiment, the nucleic acid sequence encodes a polypeptide which has at least about 90% identity with SEQ ID NO: 132. In another embodiment, the nucleic acid sequence encodes a polypeptide which has at least about 95% identity with SEQ ID NO: 132. Accordingly, in one embodiment, the nucleic acid sequence encodes a polypeptide which has as at least about 85%, 86%, 87%, 88%, 89%, 90%, 91%, 92%, 93%, 94%, 95%, 96%, 97%, 98%, or 99% identity with SEQ ID NO: 132. In another embodiment, the nucleic acid sequence encodes a polypeptide which comprises a sequence which encodes SEQ ID NO: 132. In yet another embodiment the nucleic acid sequence encodes a polypeptide which consists of a sequence which encodes SEQ ID NO: 132.

In one of the nucleic acid embodiments described herein, the propionate catabolism enzyme comprises MutA. In one embodiment, the nucleic acid sequence encodes a polypeptide, which has at least about 80% identity with SEQ ID NO: 133. In one embodiment, the nucleic acid sequence encodes a polypeptide which has at least about 90% identity with SEQ ID NO: 133. In another embodiment, the nucleic acid sequence encodes a polypeptide which has at least about 95% identity with SEQ ID NO: 133. Accordingly, in one embodiment, the nucleic acid sequence encodes a polypeptide which has at least about 85%, 86%, 87%, 88%, 89%, 90%, 91%, 92%, 93%, 94%, 95%, 96%, 97%, 98%, or 99% identity with SEQ ID NO: 133. In another embodiment, the nucleic acid sequence encodes a polypeptide which comprises a sequence which encodes SEQ ID NO: 133. In yet another embodiment the nucleic acid sequence encodes a polypeptide which consists of a sequence which encodes SEQ ID NO: 133.

In one of the nucleic acid embodiments described herein, the propionate catabolism enzyme comprises MutB. In one embodiment, the nucleic acid sequence encodes a polypeptide, which has at least about 80% identity with SEQ ID NO: 134. In one embodiment, the nucleic acid sequence encodes a polypeptide which has at least about 90% identity with SEQ ID NO: 134. In another embodiment, the nucleic acid sequence encodes a polypeptide which has at least about 95% identity with SEQ ID NO: 134. Accordingly, in one embodiment, the nucleic acid sequence encodes a polypeptide which has at least about 85%, 86%, 87%, 88%, 89%, 90%, 91%, 92%, 93%, 94%, 95%, 96%, 97%, 98%, or 99% identity with SEQ ID NO: 134. In another embodiment, the nucleic acid sequence encodes a polypeptide which comprises a sequence which encodes SEQ ID NO: 134. In yet another embodiment the nucleic acid sequence encodes a polypeptide which consists of a sequence which encodes SEQ ID NO: 134.

In one of the nucleic acid embodiments described herein, the propionate catabolism enzyme comprises AccA. In one embodiment, the nucleic acid sequence encodes a polypeptide, which has at least about 80% identity with SEQ ID NO: 130. In one embodiment, the nucleic acid sequence encodes a polypeptide which has at least about 90% identity with SEQ ID NO: 130. In another embodiment, the nucleic acid sequence encodes a polypeptide which has at least about 95% identity with SEQ ID NO: 130. Accordingly, in one embodiment, the nucleic acid sequence encodes a polypeptide which has at least about 85%, 86%, 87%, 88%, 89%, 90%, 91%, 92%, 93%, 94%, 95%, 96%, 97%, 98%, or 99% identity with SEQ ID NO: 130. In another embodiment, the nucleic acid sequence encodes a polypeptide which comprises a sequence which encodes SEQ ID NO: 130. In yet another embodiment the nucleic acid sequence encodes a polypeptide which consists of a sequence which encodes SEQ ID NO: 130.

In one of the nucleic acid embodiments described herein the propionate catabolism enzyme comprises PccB. In one embodiment, the nucleic acid sequence encodes a polypeptide, which has at least about 80% identity with SEQ ID NO: 131. In one embodiment, the nucleic acid sequence encodes a polypeptide, which has at least about 90% identity with SEQ ID NO: 131. In another embodiment, the nucleic acid sequence encodes a polypeptide, which has at least about 95% identity with SEQ ID NO: 131. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 131. In another embodiment, the nucleic acid sequence encodes a polypeptide, which comprises a sequence which encodes SEQ ID NO: 131. In yet another embodiment, the nucleic acid sequence encodes a polypeptide, which consists of a sequence which encodes SEQ ID NO: 131.

In one of the nucleic acid embodiments described herein, the propionate catabolism enzyme comprises PrpC. In one embodiment, the nucleic acid sequence encodes a polypeptide, which has at least about 80% identity with SEQ ID NO: 74. In one embodiment, the nucleic acid sequence encodes a polypeptide, which has at least about 90% identity with SEQ ID NO: 74. In another embodiment, the nucleic acid sequence encodes a polypeptide, which has at least about 95% identity with SEQ ID NO: 74. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 74. In another embodiment, the nucleic acid sequence encodes a polypeptide, which comprises a sequence which encodes SEQ ID NO: 74. In yet another embodiment, the nucleic acid sequence encodes a polypeptide, which consists of a sequence which encodes SEQ ID NO: 74.

In one of the nucleic acid embodiments described herein, the propionate catabolism enzyme comprises PrpD. In one embodiment, the nucleic acid sequence comprising encodes a polypeptide, which has at least about 80% identity with SEQ ID NO: 77. In one embodiment, the nucleic acid sequence encodes a polypeptide, which has at least about 90% identity with SEQ ID NO: 77. In another embodiment, the nucleic acid sequence encodes a polypeptide, which has at least about 95% identity with SEQ ID NO: 77. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 77. In another embodiment, the nucleic acid sequence encodes a polypeptide, which comprises a sequence which encodes SEQ ID NO: 77. In yet another embodiment, the nucleic acid sequence encodes a polypeptide, which consists of a sequence which encodes SEQ ID NO: 77.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme comprises matB (encoding Malonyl-coenzyme A (malonyl-CoA) synthetase (MatB)). Accordingly, in one embodiment the nucleic acid sequence comprising the matB gene has at least about 80% identity with SEQ ID NO: 141. Accordingly, in one embodiment, the nucleic acid sequence comprising the nucleic acid sequence comprising the matB gene has at least about 90% identity with SEQ ID NO: 141. Accordingly, in one embodiment, the nucleic acid sequence comprising the nucleic acid sequence comprising the matB gene has at least about 95% identity with SEQ ID NO: 141. Accordingly, in one embodiment, the nucleic acid sequence comprising the nucleic acid sequence comprising the matB gene has at least about 85%, 86%, 87%, 88%, 89%, 90%, 91%, 92%, 93%, 94%, 95%, 96%, 97%, 98%, or 99% identity with SEQ ID NO: 141. In another embodiment, the nucleic acid sequence comprising the nucleic acid sequence comprising the matB gene comprises the sequence of SEQ ID NO: 141. In yet another embodiment the nucleic acid sequence comprising the nucleic acid sequence comprising the matB gene consists of the sequence of SEQ ID NO: 141.

In one of the nucleic acid embodiments described herein, the propionate catabolism enzyme MatB. In one embodiment, the nucleic acid sequence encodes a polypeptide, which has at least about 89% identity with SEQ ID NO: 140. In one embodiment, the nucleic acid sequence encodes a polypeptide, which has at least about 90% identity with SEQ ID NO: 140. In another embodiment, the nucleic acid sequence encodes a polypeptide, which has at least about 95% identity with SEQ ID NO: 140. Accordingly, in one embodiment, the nucleic acid sequence encodes a polypeptide, which has at least about 85%, 86%, 89%, 88%, 89%, 90%, 91%, 92%, 93%, 94%, 95%, 96%, 97%, 98%, or 99% identity with SEQ ID NO: 140. In another embodiment, the nucleic acid sequence encodes a polypeptide, which comprises a sequence which encodes SEQ ID NO: 140. In yet another embodiment, the nucleic acid sequence encodes a polypeptide, which consists of a sequence which encodes SEQ ID NO: 140.

In one of the nucleic acid embodiments described herein, the propionate catabolism enzyme comprises PrpB. In one embodiment, the nucleic acid sequence encodes a polypeptide, which has at least about 80% identity with SEQ ID NO: 80. In one embodiment, the nucleic acid sequence encodes a polypeptide, which has at least about 90% identity with SEQ ID NO: 80. In another embodiment, the nucleic acid sequence encodes a polypeptide, which has at least about 95% identity with SEQ ID NO: 80. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 80. In another embodiment, the nucleic acid sequence encodes a polypeptide, which comprises a sequence which encodes SEQ ID NO: 80. In yet another embodiment, the nucleic acid sequence encodes a polypeptide, which consists of a sequence which encodes SEQ ID NO: 80.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises prpBCD. Accordingly, in one embodiment, the nucleic acid sequence comprising prpBCD has at least about 80% identity with SEQ ID NO: 138. In another embodiment, the nucleic acid sequence comprising prpBCD has at least about 80% identity with SEQ ID NO: 83 OR SEQ ID NO: 84. Accordingly, in one embodiment, the nucleic acid sequence comprising prpBCD has at least about 90% identity with SEQ ID NO: 138. In another embodiment, the nucleic acid sequence comprising prpBCD has at least about 90% identity with SEQ ID NO: 83 OR SEQ ID NO: 84. Accordingly, in one embodiment, the nucleic acid sequence comprising prpBCD has at least about 95% identity with SEQ ID NO: 138. In another embodiment, the nucleic acid sequence comprising prpBCD has at least about 95% identity with SEQ ID NO: 83 OR SEQ ID NO: 84. Accordingly, in one embodiment, the nucleic acid sequence comprising prpBCD has at least about 85%, 86%, 87%, 88%, 89%, 90%, 91%, 92%, 93%, 94%, 95%, 96%, 97%, 98%, or 99% identity with SEQ ID NO: 138. In another embodiment, the nucleic acid sequence comprising prpBCD has at least about 85%, 86%, 87%, 88%, 89%, 90%, 91%, 92%, 93%, 94%, 95%, 96%, 97%, 98%, or 99% identity with SEQ ID NO: 83 OR SEQ ID NO: 84. In another embodiment, the nucleic acid sequence comprising prpBCD comprises the sequence of SEQ ID NO: 138. In another embodiment, the nucleic acid sequence comprising prpBCD comprises the sequence of SEQ ID NO: 83 OR SEQ ID NO: 84. In yet another embodiment the nucleic acid sequence comprising prpBCD consists of the sequence of SEQ ID NO: 138. In another embodiment, the nucleic acid sequence comprising prpBCD consists of the sequence of SEQ ID NO: 83 OR SEQ ID NO: 84.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises a nucleic acid sequence comprising prpBCDE. Accordingly, in one embodiment, the nucleic acid sequence comprising prpBCDE has at least about 80% identity with SEQ ID NO: 55. In another embodiment, the nucleic acid sequence comprising prpBCDE has at least about 80% identity with SEQ ID NO: 93 or SEQ ID NO: 94. Accordingly, in one embodiment, the nucleic acid sequence comprising prpBCDE has at least about 90% identity with SEQ ID NO: 55. In another embodiment, the nucleic acid sequence comprising prpBCDE has at least about 90% identity with SEQ ID NO: 93 or SEQ ID NO: 94. Accordingly, in one embodiment, the nucleic acid sequence comprising prpBCDE has at least about 95% identity with SEQ ID NO: 55. In another embodiment, the nucleic acid sequence comprising prpBCDE has at least about 95% identity with SEQ ID NO: 93 or SEQ ID NO: 94. Accordingly, in one embodiment, the nucleic acid sequence comprising prpBCDE has at least about 85%, 86%, 87%, 88%, 89%, 90%, 91%, 92%, 93%, 94%, 95%, 96%, 97%, 98%, or 99% identity with SEQ ID NO: 55. In another embodiment, the nucleic acid sequence comprising prpBCDE has at least about 85%, 86%, 87%, 88%, 89%, 90%, 91%, 92%, 93%, 94%, 95%, 96%, 97%, 98%, or 99% identity with SEQ ID NO: 93 or SEQ ID NO: 94. In another embodiment, the nucleic acid sequence comprising prpBCDE comprises the sequence of SEQ ID NO: 55. In another embodiment, the nucleic acid sequence comprising prpBCDE comprises the sequence of SEQ ID NO: 93 or SEQ ID NO: 94. In yet another embodiment the nucleic acid sequence comprising prpBCDE consists of the sequence of SEQ ID NO: 55. In another embodiment, the nucleic acid sequence comprising prpBCDE consists of the sequence of SEQ ID NO: 93 or SEQ ID NO: 94.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises a nucleic acid sequence comprising phaBCA. Accordingly, in one embodiment, the nucleic acid sequence comprising phaBCA has at least about 80% identity with SEQ ID NO: 139. In one embodiment, the nucleic acid sequence comprising phaBCA has at least about 90% identity with SEQ ID NO: 139. In one embodiment, the nucleic acid sequence comprising phaBCA has at least about 95% identity with SEQ ID NO: 139. In one embodiment, the nucleic acid sequence comprising phaBCA has at least about 85%, 86%, 87%, 88%, 89%, 90%, 91%, 92%, 93%, 94%, 95%, 96%, 97%, 98%, or 99% identity with SEQ ID NO: 139. In another embodiment, the nucleic acid sequence comprising phaBCA comprises the sequence of SEQ ID NO: 139. In another embodiment, the nucleic acid sequence comprising phaBCA consists of the sequence of SEQ ID NO: 139. In one embodiment, the propionate catabolism gene cassette comprises a nucleic acid sequence comprising prpE and a nucleic acid sequence comprising phaBCA.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises nucleic acid sequence comprising phaBCA. Accordingly, in one embodiment, the nucleic acid sequence comprising phaBCA has at least about 80% identity with SEQ ID NO: 102. In one embodiment, the nucleic acid sequence comprising phaBCA has at least about 90% identity with SEQ ID NO: 102. In one embodiment, the nucleic acid sequence comprising phaBCA has at least about 95% identity with SEQ ID NO: 102. In one embodiment, the nucleic acid sequence comprising phaBCA has at least about 85%, 86%, 87%, 88%, 89%, 90%, 91%, 92%, 93%, 94%, 95%, 96%, 97%, 98%, or 99% identity with SEQ ID NO: 102. In another embodiment, the nucleic acid sequence comprising phaBCA comprises the sequence of SEQ ID NO: 102. In another embodiment, the nucleic acid sequence comprising phaBCA consists of the sequence of SEQ ID NO: 102. In one embodiment, the propionate catabolism gene cassette comprises a nucleic acid sequence comprising prpE and a nucleic acid sequence comprising phaBCA.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises a nucleic acid sequence comprising prpE-phaBCA. Accordingly, in one embodiment, the nucleic acid sequence comprising prpE-phaBCA has at least about 80% identity with SEQ ID NO: 24. In one embodiment, the nucleic acid sequence comprising prpE-phaBCA has at least about 90% identity with SEQ ID NO: 24. In one embodiment, the nucleic acid sequence comprising prpE-phaBCA has at least about 95% identity with SEQ ID NO: 24. In one embodiment, the nucleic acid sequence comprising prpE-phaBCA has at least about 85%, 86%, 87%, 88%, 89%, 90%, 91%, 92%, 93%, 94%, 95%, 96%, 97%, 98%, or 99% identity with SEQ ID NO: 24. In another embodiment, the nucleic acid sequence comprising prpE-phaBCA comprises the sequence of SEQ ID NO: 24. In another embodiment, the nucleic acid sequence comprising prpE-phaBCA consists of the sequence of SEQ ID NO: 24.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises a nucleic acid sequence comprising prpE, pccB, accA1, mmcE, mutA, and mutB. Accordingly, in one embodiment, the nucleic acid sequence comprising prpE-pccB-accA1-mmcE-mutA-mutB has at least about 80% identity with a combination of SEQ ID NO: 37 and 31. In one embodiment, the nucleic acid sequence comprising prpE-pccB-accA1-mmcE-mutA-mutB has at least about 90% identity with a combination of SEQ ID NO: 37 and 31. In one embodiment, the nucleic acid sequence comprising prpE-pccB-accA1-mmcE-mutA-mutB has at least about 95% identity with a combination of SEQ ID NO: 37 and 31. In one embodiment, the nucleic acid sequence comprising prpE-pccB-accA1-mmcE-mutA-mutB has at least about 85%, 86%, 87%, 88%, 89%, 90%, 91%, 92%, 93%, 94%, 95%, 96%, 97%, 98%, or 99% identity with a combination of SEQ ID NO: 37 and 31. In another embodiment, the nucleic acid sequence comprising prpE-pccB-accA1-mmcE-mutA-mutB comprises the sequence of a combination of SEQ ID NO: 37 and 31. In another embodiment, the nucleic acid sequence comprising prpE-pccB-accA1-mmcE-mutA-mutB consists of the sequence of a combination of SEQ ID NO: 37 and 31.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises a nucleic acid sequence comprising prpE, pccB, and accA1. Accordingly, in one embodiment, nucleic acid sequence comprising the prpE-pccB-accA1 has at least about 80% identity with SEQ ID NO: 37. In one embodiment, the nucleic acid sequence comprising prpE-pccB-accA1 has at least about 90% identity with SEQ ID NO: 37. In one embodiment, the nucleic acid sequence comprising prpE-pccB-accA1 has at least about 95% identity with SEQ ID NO: 37. In one embodiment, the nucleic acid sequence comprising prpE-pccB-accA1 has at least about 85%, 86%, 87%, 88%, 89%, 90%, 91%, 92%, 93%, 94%, 95%, 96%, 97%, 98%, or 99% identity with SEQ ID NO: 37. In another embodiment, the nucleic acid sequence comprising prpE-pccB-accA1 comprises the sequence of SEQ ID NO: 37. In another embodiment, the nucleic acid sequence comprising prpE-pccB-accA1 consists of the sequence of SEQ ID NO: 37.

In some embodiments, the disclosure provides novel nucleic acids for metabolizing propionate. In some embodiments, the nucleic acid comprises gene sequence encoding one or more propionate catabolism enzyme cassette(s). In one of the nucleic acid embodiments described herein, the nucleic acid sequence encoding the propionate catabolism enzyme cassette comprises a nucleic acid sequence comprising mmcE, mutA, and mutB. Accordingly, in one embodiment, the nucleic acid sequence comprising mmcE-mutA-mutB has at least about 80% identity with a combination of SEQ ID NO:31. In one embodiment, the nucleic acid sequence comprising mmcE-mutA-mutB has at least about 90% identity with a combination of SEQ ID NO: 31. In one embodiment, the nucleic acid sequence comprising mmcE-mutA-mutB has at least about 95% identity with a combination of SEQ ID NO: 31. In one embodiment, the nucleic acid sequence comprising mmcE-mutA-mutB has at least about 85%, 86%, 87%, 88%, 89%, 90%, 91%, 92%, 93%, 94%, 95%, 96%, 97%, 98%, or 99% identity with a combination of SEQ ID NO: 31. In another embodiment, the nucleic acid sequence comprising mmcE-mutA-mutB comprises the sequence of a combination of SEQ ID NO: 31. In another embodiment, the nucleic acid sequence comprising mmcE-mutA-mutB consists of the sequence of a combination of SEQ ID NO: 31.

In some embodiments, the disclosure provides novel nucleic acids for metabolizing propionate. In some embodiments, the nucleic acid comprises gene sequence encoding one or more propionate catabolism enzyme(s). In one of the nucleic acid embodiments described herein, the nucleic acid sequence encoding the propionate catabolism enzyme cassette comprises cobB (encoding CobB, a NAD-dependent deacylase). In one embodiment, nucleic acid sequence comprising the cobB gene has at least about 80% identity with SEQ ID NO: 114. Accordingly, in one embodiment, nucleic acid sequence comprising the cobB gene has at least about 90% identity with SEQ ID NO: 114. Accordingly, in one embodiment, nucleic acid sequence comprising the cobB gene has at least about 95% identity with SEQ ID NO: 114. Accordingly, in one embodiment, nucleic acid sequence comprising the cobB gene has at least about 85%, 86%, 87%, 88%, 89%, 90%, 91%, 92%, 93%, 94%, 95%, 96%, 97%, 98%, or 99% identity with SEQ ID NO: 114. In another embodiment, nucleic acid sequence comprising the cobB gene comprises the sequence of SEQ ID NO: 114. In yet another embodiment nucleic acid sequence comprising the cobB gene consists of the sequence of SEQ ID NO: 114.

In one of the nucleic acid embodiments described herein, the propionate catabolism enzyme comprises CobB. In one embodiment, the nucleic acid sequence encodes a polypeptide, which has at least about 80% identity with SEQ ID NO: 113. In one embodiment, the nucleic acid sequence encodes a polypeptide, which has at least about 90% identity with SEQ ID NO: 113. In another embodiment, the nucleic acid sequence encodes a polypeptide, which has at least about 95% identity with SEQ ID NO: 113. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 113. In another embodiment, the nucleic acid sequence encodes a polypeptide, which comprises a sequence which encodes SEQ ID NO: 113. In yet another embodiment, the nucleic acid sequence encodes a polypeptide, which consists of a sequence which encodes SEQ ID NO: 113.

In some embodiments, the disclosure provides novel nucleic acids for transporting propionate. In some embodiments, the nucleic acid comprises gene sequence encoding one or more propionate catabolism enzyme(s). In one of the nucleic acid embodiments described herein, the nucleic acid sequence comprises mctC (encoding the propionate importer MctC). In one embodiment, nucleic acid sequence comprising the mctC gene has at least about 80% identity to SEQ ID NO: 88. Accordingly, in one embodiment, nucleic acid sequence comprising the mctC gene has at least about 90% identity to SEQ ID NO: 88. Accordingly, in one embodiment, nucleic acid sequence comprising the mctC gene has at least about 95% identity to SEQ ID NO: 88. Accordingly, in one embodiment, nucleic acid sequence comprising the mctC gene has at least about 85%, 86%, 87%, 88%, 89%, 90%, 91%, 92%, 93%, 94%, 95%, 96%, 97%, 98%, or 99% identity to SEQ ID NO: 88. In another embodiment, nucleic acid sequence comprising the mctC gene comprises the sequence of SEQ ID NO: 88. In yet another embodiment nucleic acid sequence comprising the mctC gene consists of the sequence of SEQ ID NO: 88.

In one of the nucleic acid embodiments described herein, the propionate transporter comprises MctC. In one embodiment, the nucleic acid sequence encodes a polypeptide which has at least about 80% identity with SEQ ID NO: 87. In one embodiment, the nucleic acid sequence encodes a polypeptide, which has at least about 90% identity with SEQ ID NO: 87. In another embodiment, the nucleic acid sequence encodes a polypeptide, which has at least about 95% identity with SEQ ID NO: 87. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 87. In another embodiment, the nucleic acid sequence encodes a polypeptide, which comprises a sequence which encodes SEQ ID NO: 87. In yet another embodiment, the nucleic acid sequence encodes a polypeptide, which consists of a sequence which encodes SEQ ID NO: 87.

In some embodiments, the disclosure provides novel nucleic acids for transporting propionate into the cell. In some embodiments, the nucleic acid comprises gene sequence encoding one or more propionate catabolism enzyme(s). In one of the nucleic acid embodiments described herein, the nucleic acid sequence comprises putP_6 (encoding the propionate importer PutP_6). In one embodiment, nucleic acid sequence comprising the putP_6 gene has at least about 80% identity to SEQ ID NO: 90. Accordingly, in one embodiment, nucleic acid sequence comprising the putP_6 gene has at least about 90% identity to SEQ ID NO: 90. Accordingly, in one embodiment, nucleic acid sequence comprising the putP_6 gene has at least about 95% identity to SEQ ID NO: 90. Accordingly, in one embodiment, nucleic acid sequence comprising the putP_6 gene has at least about 85%, 86%, 87%, 88%, 89%, 90%, 91%, 92%, 93%, 94%, 95%, 96%, 97%, 98%, or 99% identity to SEQ ID NO: 90. In another embodiment, nucleic acid sequence comprising the putP_6 gene comprises the sequence of SEQ ID NO: 90. In yet another embodiment nucleic acid sequence comprising the putP_6 gene consists of the sequence of SEQ ID NO: 90.

In one of the nucleic acid embodiments described herein, the propionate transporter comprises PutP_6. In one embodiment, the nucleic acid sequence encodes a polypeptide, which has at least about 80% identity with SEQ ID NO: 89. In one embodiment, the nucleic acid sequence encodes a polypeptide, which has at least about 90% identity with SEQ ID NO: 89. In another embodiment, the nucleic acid sequence encodes a polypeptide, which has at least about 95% identity with SEQ ID NO: 89. Accordingly, in one embodiment, the nucleic acid sequence encodes a polypeptide, which has at least about 85%, 86%, 89%, 88%, 89%, 90%, 91%, 92%, 93%, 94%, 95%, 96%, 97%, 98%, or 99% identity with SEQ ID NO: 89. In another embodiment, the nucleic acid sequence encodes a polypeptide, which comprises a sequence which encodes SEQ ID NO: 89. In yet another embodiment, the nucleic acid sequence encodes a polypeptide, which consists of a sequence which encodes SEQ ID NO: 89.

In some embodiments, the disclosure provides novel nucleic acids for exporting succinate. In some embodiments, the nucleic acid comprises gene sequence encoding one or more succinate exporter(s). In one of the nucleic acid embodiments described herein, the nucleic acid sequence encoding the succinate exporter comprises dcuC (encoding the succinate exporter DcuC). In one embodiment, the nucleic acid sequence comprising the dcuC gene has at least about 80% identity to SEQ ID NO: 49. Accordingly, in one embodiment, the nucleic acid sequence comprising the dcuC gene has at least about 90% identity to SEQ ID NO: 49. Accordingly, in one embodiment, the nucleic acid sequence comprising the dcuC gene has at least about 95% identity to SEQ ID NO: 49. Accordingly, in one embodiment, the nucleic acid sequence comprising the dcuC gene has at least about 85%, 86%, 87%, 88%, 89%, 90%, 91%, 92%, 93%, 94%, 95%, 96%, 97%, 98%, or 99% identity to SEQ ID NO: 49. In another embodiment, the nucleic acid sequence comprising the dcuC gene comprises the sequence of SEQ ID NO: 49. In yet another embodiment the nucleic acid sequence comprising the dcuC gene consists of the sequence of SEQ ID NO:70.

In one of the nucleic acid embodiments described herein, the succinate exporter comprises DcuC. In one embodiment, the nucleic acid sequence encodes a polypeptide, which has at least about 80% identity with SEQ ID NO: 129. In one embodiment, the nucleic acid sequence encodes a polypeptide, which has at least about 90% identity with SEQ ID NO: 129. In another embodiment, the nucleic acid sequence encodes a polypeptide, which has at least about 95% identity with SEQ ID NO: 129. Accordingly, in one embodiment, the nucleic acid sequence encodes a polypeptide, which has at least about 85%, 86%, 89%, 88%, 89%, 90%, 91%, 92%, 93%, 94%, 95%, 96%, 97%, 98%, or 99% identity with SEQ ID NO: 129. In another embodiment, the nucleic acid sequence encodes a polypeptide, which comprises a sequence which encodes SEQ ID NO: 129. In yet another embodiment, the nucleic acid sequence encodes a polypeptide, which consists of a sequence which encodes SEQ ID NO: 129.

In some embodiments, the disclosure provides novel nucleic acids for metabolizing propionate. In some embodiments, the nucleic acid comprises gene sequence encoding one or more succinate exporter(s). In one of the nucleic acid embodiments described herein, the nucleic acid sequence comprises dcuC. In one embodiment, the nucleic acid sequence comprising the dcuC gene has at least about 80% identity to SEQ ID NO: 118. Accordingly, in one embodiment, the nucleic acid sequence comprising the dcuC gene has at least about 90% identity to SEQ ID NO: 118. Accordingly, in one embodiment, the nucleic acid sequence comprising the dcuC gene has at least about 95% identity to SEQ ID NO: 118. Accordingly, in one embodiment, the nucleic acid sequence comprising the dcuC gene has at least about 85%, 86%, 87%, 88%, 89%, 90%, 91%, 92%, 93%, 94%, 95%, 96%, 97%, 98%, or 99% identity to SEQ ID NO: 118. In another embodiment, the nucleic acid sequence comprising the dcuC gene comprises the sequence of SEQ ID NO: 118. In yet another embodiment the nucleic acid sequence comprising the dcuC gene consists of the sequence of SEQ ID NO: 118.

In one of the nucleic acid embodiments described herein, the propionate transporter enzyme comprises DcuC. In one embodiment, the nucleic acid encodes a polypeptide, which has at least about 80% identity with SEQ ID NO: 117. In one embodiment, the nucleic acid sequence encodes a polypeptide, which has at least about 90% identity with SEQ ID NO: 117. In another embodiment, the nucleic acid sequence encodes a polypeptide, which has at least about 95% identity with SEQ ID NO: 117. Accordingly, in one embodiment, the nucleic acid sequence encodes a polypeptide, which has at least about 85%, 86%, 89%, 88%, 89%, 90%, 91%, 92%, 93%, 94%, 95%, 96%, 97%, 98%, or 99% identity with SEQ ID NO: 117. In another embodiment, the nucleic acid sequence encodes a polypeptide, which comprises a sequence which encodes SEQ ID NO: 117. In yet another embodiment, the nucleic acid sequence encodes a polypeptide, which consists of a sequence which encodes SEQ ID NO: 117.

In some embodiments, the disclosure provides novel nucleic acids for metabolizing propionate. In some embodiments, the nucleic acid comprises gene sequence encoding one or more succinate exporter(s). In one of the nucleic acid embodiments described herein, the nucleic acid sequence comprises sucE1 (encoding the succinate exporter SucE1). In one embodiment, nucleic acid sequence comprising the sucE1 gene has at least about 80% identity to SEQ ID NO: 46. Accordingly, in one embodiment, nucleic acid sequence comprising the sucE1 gene has at least about 90% identity to SEQ ID NO: 46. Accordingly, in one embodiment, nucleic acid sequence comprising the sucE1 gene has at least about 95% identity to SEQ ID NO: 46. Accordingly, in one embodiment, nucleic acid sequence comprising the sucE1 gene has at least about 85%, 86%, 87%, 88%, 89%, 90%, 91%, 92%, 93%, 94%, 95%, 96%, 97%, 98%, or 99% identity to SEQ ID NO: 46. In another embodiment, nucleic acid sequence comprising the sucE1 gene comprises the sequence of SEQ ID NO: 46. In yet another embodiment nucleic acid sequence comprising the sucE1 gene consists of the sequence of SEQ ID NO: 46.

In some embodiments, the disclosure provides novel nucleic acids for metabolizing propionate. In some embodiments, the nucleic acid comprises gene sequence encoding one or more succinate exporter(s). In one of the nucleic acid embodiments described herein, the nucleic acid sequence comprises sucE1. In one embodiment, nucleic acid sequence comprising the sucE1 gene has at least about 80% identity to SEQ ID NO: 120. Accordingly, in one embodiment, nucleic acid sequence comprising the sucE1 gene has at least about 90% identity to SEQ ID NO: 120. Accordingly, in one embodiment, nucleic acid sequence comprising the sucE1 gene has at least about 95% identity to SEQ ID NO: 120. Accordingly, in one embodiment, nucleic acid sequence comprising the sucE1 gene has at least about 85%, 86%, 87%, 88%, 89%, 90%, 91%, 92%, 93%, 94%, 95%, 96%, 97%, 98%, or 99% identity to SEQ ID NO: 120. In another embodiment, nucleic acid sequence comprising the sucE1 gene comprises the sequence of SEQ ID NO: 120. In yet another embodiment nucleic acid sequence comprising the sucE1 gene consists of the sequence of SEQ ID NO: 120.

In one of the nucleic acid embodiments described herein, the succinate exporter comprises sucE1. In one embodiment, the nucleic acid sequence encodes a polypeptide which has at least about 80% identity with SEQ ID NO: 128. In one embodiment, the nucleic acid sequence encodes a polypeptide, which has at least about 90% identity with SEQ ID NO: 128. In another embodiment, the nucleic acid sequence encodes a polypeptide, which has at least about 95% identity with SEQ ID NO: 128. Accordingly, in one embodiment, the nucleic acid sequence encodes a polypeptide, which has at least about 85%, 86%, 89%, 88%, 89%, 90%, 91%, 92%, 93%, 94%, 95%, 96%, 97%, 98%, or 99% identity with SEQ ID NO: 128. In another embodiment, the nucleic acid sequence encodes a polypeptide, which comprises a sequence which encodes SEQ ID NO: 128. In yet another embodiment, the nucleic acid sequence encodes a polypeptide, which consists of a sequence which encodes SEQ ID NO: 128.

In one embodiment, the nucleic acid sequence encodes a polypeptide which has at least about 80% identity with SEQ ID NO: 119. In one embodiment, the nucleic acid sequence encodes a polypeptide, which has at least about 90% identity with SEQ ID NO: 119. In another embodiment, the nucleic acid sequence encodes a polypeptide, which has at least about 95% identity with SEQ ID NO: 119. Accordingly, in one embodiment, the nucleic acid sequence encodes a polypeptide, which has at least about 85%, 86%, 89%, 88%, 89%, 90%, 91%, 92%, 93%, 94%, 95%, 96%, 97%, 98%, or 99% identity with SEQ ID NO: 119. In another embodiment, the nucleic acid sequence encodes a polypeptide, which comprises a sequence which encodes SEQ ID NO: 119. In yet another embodiment, the nucleic acid sequence encodes a polypeptide, which consists of a sequence which encodes SEQ ID NO: 119.

In any of the nucleic acid embodiments described herein, the gene sequence encoding one or more molecules that metabolize propionic acid is operably linked to an inducible promoter. In any of the nucleic acid embodiments described herein, the gene sequence encoding one or more molecules that metabolize propionic acid are operably linked to an inducible promoter that is directly or indirectly induced by exogenous environmental conditions. In some embodiments, gene sequence encoding one or more molecules that metabolize propionic acid is operably linked to an oxygen level-dependent promoter (e.g., FNR-inducible promoter). In some embodiments, gene sequence encoding one or more molecules that metabolize propionic acid is operably linked to a promoter induced by inflammation or an inflammatory response (RNS, ROS promoters: In some embodiments, gene sequence encoding one or more molecules that metabolize propionic acid is operably linked to a promoter induced by a metabolite that may or may not be naturally present (e.g., can be exogenously added) in the gut, e.g., arabinose is used. In some embodiments, gene sequence encoding one or more molecules that metabolize propionic acid is operably linked to a promoter induced during cell culture, expansion and/or manufacture.

In any of the nucleic acid embodiments described herein, the gene sequence encoding one or more molecules that metabolize propionic acid is operably linked to a constitutive promoter. For example, in any of the nucleic acid embodiments described herein, the gene sequence encoding one or more molecules that metabolize propionic acid is operably linked to constitutive promoter disclosed herein or otherwise known in the art. In any of the nucleic acid embodiments described herein, the gene sequence encoding one or more molecules that metabolize propionic acid is operably linked to constitutive promoter provided in Tables 8-18.

In any of the nucleic acid embodiments described herein, the gene sequence encoding one or more molecules that metabolize propionic acid is linked to any constitutive or inducible promoter described herein.

In any of the nucleic acid embodiments described herein, the gene sequence encoding one or more molecules that transport propionic acid is operably linked to an inducible promoter. In any of the nucleic acid embodiments described herein, the gene sequence encoding one or more molecules that transport propionic acid are operably linked to an inducible promoter that is directly or indirectly induced by exogenous environmental conditions. In some embodiments, gene sequence encoding one or more molecules that transport propionic acid is operably linked to an oxygen level-dependent promoter (e.g., FNR-inducible promoter). In some embodiments, gene sequence encoding one or more molecules that transport propionic acid is operably linked to a promoter induced by inflammation or an inflammatory response (RNS, ROS promoters: In some embodiments, gene sequence encoding one or more molecules that transport propionic acid is operably linked to a promoter induced by a metabolite that may or may not be naturally present (e.g., can be exogenously added) in the gut, e.g., arabinose is used. In some embodiments, gene sequence encoding one or more molecules that transport propionic acid is operably linked to a promoter induced during cell culture, expansion and/or manufacture.

In any of the nucleic acid embodiments described herein, the gene sequence encoding one or more molecules that transport propionic acid is operably linked to a constitutive promoter. For example, in any of the nucleic acid embodiments described herein, the gene sequence encoding one or more molecules that transport propionic acid is operably linked to constitutive promoter disclosed herein or otherwise known in the art. In any of the nucleic acid embodiments described herein, the gene sequence encoding one or more molecules that transport propionic acid is operably linked to constitutive promoter provided in Tables 8-18.

In any of the nucleic acid embodiments described herein, the gene sequence encoding one or more molecules that metabolize propionic acid is linked to any constitutive or inducible promoter described herein.

In any of the nucleic acid embodiments described herein, the gene sequence encoding one or more molecules that export succinate is operably linked to an inducible promoter. In any of the nucleic acid embodiments described herein, the gene sequence encoding one or more molecules that export succinate are operably linked to an inducible promoter that is directly or indirectly induced by exogenous environmental conditions. In some embodiments, gene sequence encoding one or more molecules that export succinate is operably linked to an oxygen level-dependent promoter (e.g., FNR-inducible promoter). In some embodiments, gene sequence encoding one or more molecules that export succinate is operably linked to a promoter induced by inflammation or an inflammatory response (RNS, ROS promoters \_{L} In some embodiments, gene sequence encoding one or more molecules that export succinate is operably linked to a promoter induced by a metabolite that may or may not be naturally present (e.g., can be exogenously added) in the gut, e.g., arabinose is used. In some embodiments, gene sequence encoding one or more molecules that export succinate is operably linked to a promoter induced during cell culture, expansion and/or manufacture.

In any of the nucleic acid embodiments described herein, the gene sequence encoding one or more molecules that export succinate is operably linked to a constitutive promoter. For example, in any of the nucleic acid embodiments described herein, the gene sequence encoding one or more molecules that export succinate is operably linked to constitutive promoter disclosed herein or otherwise known in the art. In any of the nucleic acid embodiments described herein, the gene sequence encoding one or more molecules that metabolize export succinate is operably linked to constitutive promoter provided in Tables 8-18.

In any of the nucleic acid embodiments described herein, the gene sequence encoding one or more molecules that export succinate is linked to any constitutive or inducible promoter described herein.

In one embodiment, the at least one gene encoding an exporter of succinate is located on a plasmid in the bacterial cell. In another embodiment, the at least one gene encoding an exporter of succinate is located in the chromosome of the bacterial cell. In yet another embodiment, a native copy of the at least one gene encoding an exporter of succinate is located in the chromosome of the bacterial cell, and a copy of at least one gene encoding an exporter of succinate from a different species of bacteria is located on a plasmid in the bacterial cell. In yet another embodiment, a native copy of the at least one gene encoding an exporter of a succinate is located on a plasmid in the bacterial cell, and a copy of at least one gene encoding an exporter of succinate from a different species of bacteria is located on a plasmid in the bacterial cell. In yet another embodiment, a native copy of the at least one gene encoding an exporter of succinate is located in the chromosome of the bacterial cell, and a copy of the at least one gene encoding an exporter of succinate from a different species of bacteria is located in the chromosome of the bacterial cell.

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the engineered bacteria of the disclosure becoming an auxotroph, e.g., the bacteria may be an auxotroph depending on the environmental conditions (a conditional auxotroph). An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth. In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil, as long as the corresponding wild-type gene product is not produced in the bacteria.

Table 19 lists depicts exemplary bacterial genes which may be disrupted or deleted to produce an auxotrophic strain. These include, but are not limited to, genes required for oligonucleotide synthesis, amino acid synthesis, and cell wall synthesis.

TABLE 19
Non-limiting Examples of Bacterial Genes Useful for
Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 20 shows the survival of various amino acid auxotrophs in the mouse gut, as detected 24 hrs and 48 hrs post-gavage. These auxotrophs were generated using BW25113, a non-Nissle strain of E. coli.

TABLE 20
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| serA | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/ | Present | Present | Absent |
| | Leucine | | | |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thymidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, ftsB, eno, pyrG, chpR, lgt, fbaA, pgk, yqgD, metK, yqgF, plsC, ygiT, pare, ribB, cca, ygjD, tdcF, yraL, yihA, ftsN, murl, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, lspA, ispH, dapB, folA, imp, yabQ, ftsL, ftsl, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsl, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, int, glnS, fldA, cydA, infA, cydC, ftsK, lolA, serS, rpsA, msbA, lpxK, kdsB, mukF, mukE, mukB, asnS, fabA, mviN, me, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabl, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the engineered bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4: 307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

Genetic Regulatory Circuits

In some embodiments, the genetically engineered bacteria comprise multi-layered genetic regulatory circuits for expressing the constructs described herein (see, e.g., U.S. Provisional Application No. 62/184,811, incorporated herein by reference in its entirety). The genetic regulatory circuits are useful to screen for mutant bacteria that produce a propionate catabolism enzyme, propionate transporter, and/or propionate binding protein or rescue an auxotroph. In certain embodiments, the invention provides methods for selecting genetically engineered bacteria that produce one or more genes of interest.

In some embodiments, the invention provides genetically engineered bacteria comprising a gene or gene cassette for producing a payload and a T7 polymerase-regulated genetic regulatory circuit. For example, the genetically engineered bacteria comprise a first gene encoding a T7 polymerase, wherein the first gene is operably linked to a fumarate and nitrate reductase regulator (FNR)-responsive promoter; a second gene or gene cassette for producing a payload, wherein the second gene or gene cassette is operably linked to a T7 promoter that is induced by the T7 polymerase; and a third gene encoding an inhibitory factor, lysY, that is capable of inhibiting the T7 polymerase. In the presence of oxygen, FNR does not bind the FNR-responsive promoter, and the payload is not expressed. LysY is expressed constitutively (P-lac constitutive) and further inhibits T7 polymerase. In the absence of oxygen, FNR dimerizes and binds to the FNR-responsive promoter, T7 polymerase is expressed at a level sufficient to overcome lysY inhibition, and the payload is expressed. In some embodiments, the lysY gene is operably linked to an additional FNR binding site. In the absence of oxygen, FNR dimerizes to activate T7 polymerase expression as described above, and also inhibits lysY expression.

In some embodiments, the invention provides genetically engineered bacteria comprising a gene or gene cassette for producing a payload and a protease-regulated genetic regulatory circuit. For example, the genetically engineered bacteria comprise a first gene encoding an mf-lon protease, wherein the first gene is operably linked to a FNR-responsive promoter; a second gene or gene cassette for producing a payload operably linked to a tet regulatory region (tetO); and a third gene encoding an mf-lon degradation signal linked to a tet repressor (tetR), wherein the tetR is capable of binding to the tet regulatory region and repressing expression of the second gene or gene cassette. The mf-lon protease is capable of recognizing the mf-lon degradation signal and degrading the tetR. In the presence of oxygen, FNR does not bind the FNR-responsive promoter, the repressor is not degraded, and the payload is not expressed. In the absence of oxygen, FNR dimerizes and binds the FNR-responsive promoter, thereby inducing expression of mf-lon protease. The mf-lon protease recognizes the mf-lon degradation signal and degrades the tetR, and the payload is expressed.

In some embodiments, the invention provides genetically engineered bacteria comprising a gene or gene cassette for producing a payload and a repressor-regulated genetic regulatory circuit. For example, the genetically engineered bacteria comprise a first gene encoding a first repressor, wherein the first gene is operably linked to a FNR-responsive promoter; a second gene or gene cassette for producing a payload operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the payload is not expressed. In the absence of oxygen, FNR dimerizes and binds the FNR-responsive promoter, the first repressor is expressed, the second repressor is not expressed, and the payload is expressed.

Examples of repressors useful in these embodiments include, but are not limited to, ArgR, TetR, ArsR, AscG, LacI, CscR, DeoR, DgoR, FruR, GalR, GatR, CI, LexA, RafR, QacR, and PtxS (US20030166191).

In some embodiments, the invention provides genetically engineered bacteria comprising a gene or gene cassette for producing a payload and a regulatory RNA-regulated genetic regulatory circuit. For example, the genetically engineered bacteria comprise a first gene encoding a regulatory RNA, wherein the first gene is operably linked to a FNR-responsive promoter, and a second gene or gene cassette for producing a payload. The second gene or gene cassette is operably linked to a constitutive promoter and further linked to a nucleotide sequence capable of producing an mRNA hairpin that inhibits translation of the payload. The regulatory RNA is capable of eliminating the mRNA hairpin and inducing payload translation via the ribosomal binding site. In the presence of oxygen, FNR does not bind the FNR-responsive promoter, the regulatory RNA is not expressed, and the mRNA hairpin prevents the payload from being translated. In the absence of oxygen, FNR dimerizes and binds the FNR-responsive promoter, the regulatory RNA is expressed, the mRNA hairpin is eliminated, and the payload is expressed.

In some embodiments, the invention provides genetically engineered bacteria comprising a gene or gene cassette for producing a payload and a CRISPR-regulated genetic regulatory circuit. For example, the genetically engineered bacteria comprise a Cas9 protein; a first gene encoding a CRISPR guide RNA, wherein the first gene is operably linked to a FNR-responsive promoter; a second gene or gene cassette for producing a payload, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the payload is not expressed. In the absence of oxygen, FNR dimerizes and binds the FNR-responsive promoter, the guide RNA is expressed, the repressor is not expressed, and the payload is expressed.

In some embodiments, the invention provides genetically engineered bacteria comprising a gene or gene cassette for producing a payload and a recombinase-regulated genetic regulatory circuit. For example, the genetically engineered bacteria comprise a first gene encoding a recombinase, wherein the first gene is operably linked to a FNR-responsive promoter, and a second gene or gene cassette for producing a payload operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the payload remains in the 3′ to 5′ orientation, and no functional payload is produced. In the absence of oxygen, FNR dimerizes and binds the FNR-responsive promoter, the recombinase is expressed, the payload is reverted to the 5′ to 3′ orientation, and functional payload is produced.

In some embodiments, the invention provides genetically engineered bacteria comprising a gene or gene cassette for producing a payload and a polymerase- and recombinase-regulated genetic regulatory circuit. For example, the genetically engineered bacteria comprise a first gene encoding a recombinase, wherein the first gene is operably linked to a FNR-responsive promoter; a second gene or gene cassette for producing a payload operably linked to a T7 promoter; a third gene encoding a T7 polymerase, wherein the T7 polymerase is capable of binding to the T7 promoter and inducing expression of the payload. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the payload is not expressed. In the absence of oxygen, FNR dimerizes and binds the FNR-responsive promoter, the recombinase is expressed, the T7 polymerase gene is reverted to the 5′ to 3′ orientation, and the payload is expressed.

Kill Switches

In some embodiments, the genetically engineered bacteria also comprise a kill switch (see, e.g., U.S. Provisional Application Nos. 62/183,935 and 62/263,329, each of which are expressly incorporated herein by reference in their entireties). The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

Bacteria engineered with kill switches have been engineered for in vitro research purposes, e.g., to limit the spread of a biofuel-producing microorganism outside of a laboratory environment. Bacteria engineered for in vivo administration to treat a disease or disorder may also be programmed to die at a specific time after the expression and delivery of a heterologous gene, genes or gene cassette(s), for example, a therapeutic gene(s) or after the subject has experienced the therapeutic effect. For example, in some embodiments, the kill switch is activated to kill the bacteria after a period of time following expression of the propionate catabolism enzyme cassette(s) and/or gene(s) present in the engineered bacteria. In some embodiments, the kill switch is activated in a delayed fashion following expression of the heterologous gene(s) or gene cassette(s), for example, after the production of the corresponding protein(s) or molecule(s). Alternatively, the bacteria may be engineered to die after the bacteria has spread outside of a disease site. Specifically, it may be useful to prevent long-term colonization of subjects by the microorganism, spread of the microorganism outside the area of interest (for example, outside the gut) within the subject, or spread of the microorganism outside of the subject into the environment (for example, spread to the environment through the stool of the subject).

Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al., 2000), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al., 2010). In some embodiments, the kill switch is activated to kill the bacteria after a period of time following oxygen level-dependent expression of a heterologous gene(s) or gene cassette(s). In some embodiments, the kill switch is activated in a delayed fashion following oxygen level-dependent expression of a heterologous gene(s) or gene cassette(s).

Kill-switches can be designed such that a toxin is produced in response to an environmental condition or external signal (e.g., the bacteria is killed in response to an external cue; i.e., an activation-based kill switch) or, alternatively designed such that a toxin is produced once an environmental condition no longer exists or an external signal is ceased (i.e., a repression-based kill switch).

Thus, in some embodiments, the genetically engineered bacteria of the disclosure are further programmed to die after sensing an exogenous environmental signal, for example, in a low oxygen environment. In some embodiments, the genetically engineered bacteria of the present disclosure comprise one or more genes encoding one or more recombinase(s), whose expression is induced in response to an environmental condition or signal and causes one or more recombination events that ultimately leads to the expression of a toxin which kills the cell. In some embodiments, the at least one recombination event is the flipping of an inverted heterologous gene encoding a bacterial toxin which is then constitutively expressed after it is flipped by the first recombinase. In one embodiment, constitutive expression of the bacterial toxin kills the genetically engineered bacterium. In these types of kill-switch systems once the engineered bacterial cell senses the exogenous environmental condition and expresses the heterologous gene of interest, the engineered bacterial cell is no longer viable.

In another embodiment in which the genetically engineered bacteria of the present disclosure express one or more recombinase(s) in response to an environmental condition or signal causing at least one recombination event, the genetically engineered bacterium further expresses a heterologous gene encoding an anti-toxin in response to an exogenous environmental condition or signal. In one embodiment, the at least one recombination event is flipping of an inverted heterologous gene encoding a bacterial toxin by a first recombinase. In one embodiment, the inverted heterologous gene encoding the bacterial toxin is located between a first forward recombinase recognition sequence and a first reverse recombinase recognition sequence. In one embodiment, the heterologous gene encoding the bacterial toxin is constitutively expressed after it is flipped by the first recombinase. In one embodiment, the anti-toxin inhibits the activity of the toxin, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In another embodiment, the at least one recombination event is flipping of an inverted heterologous gene encoding a second recombinase by a first recombinase, followed by the flipping of an inverted heterologous gene encoding a bacterial toxin by the second recombinase. In one embodiment, the inverted heterologous gene encoding the second recombinase is located between a first forward recombinase recognition sequence and a first reverse recombinase recognition sequence. In one embodiment, the inverted heterologous gene encoding the bacterial toxin is located between a second forward recombinase recognition sequence and a second reverse recombinase recognition sequence. In one embodiment, the heterologous gene encoding the second recombinase is constitutively expressed after it is flipped by the first recombinase. In one embodiment, the heterologous gene encoding the bacterial toxin is constitutively expressed after it is flipped by the second recombinase. In one embodiment, the genetically engineered bacterium is killed by the bacterial toxin. In one embodiment, the genetically engineered bacterium further expresses a heterologous gene encoding an anti-toxin in response to the exogenous environmental condition. In one embodiment, the anti-toxin inhibits the activity of the toxin when the exogenous environmental condition is present, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In one embodiment, the at least one recombination event is flipping of an inverted heterologous gene encoding a second recombinase by a first recombinase, followed by flipping of an inverted heterologous gene encoding a third recombinase by the second recombinase, followed by flipping of an inverted heterologous gene encoding a bacterial toxin by the third recombinase. Accordingly, in one embodiment, the disclosure provides at least 1, 2, 3, 4, 5, 6, 7, 8, 9, 10, 11, 12, 13, 14, 15, 16, 17, 18, 19, or 20 recombinases that can be used serially.

In one embodiment, the at least one recombination event is flipping of an inverted heterologous gene encoding a first excision enzyme by a first recombinase. In one embodiment, the inverted heterologous gene encoding the first excision enzyme is located between a first forward recombinase recognition sequence and a first reverse recombinase recognition sequence. In one embodiment, the heterologous gene encoding the first excision enzyme is constitutively expressed after it is flipped by the first recombinase. In one embodiment, the first excision enzyme excises a first essential gene. In one embodiment, the programmed engineered bacterial cell is not viable after the first essential gene is excised.

In one embodiment, the first recombinase further flips an inverted heterologous gene encoding a second excision enzyme. In one embodiment, the wherein the inverted heterologous gene encoding the second excision enzyme is located between a second forward recombinase recognition sequence and a second reverse recombinase recognition sequence. In one embodiment, the heterologous gene encoding the second excision enzyme is constitutively expressed after it is flipped by the first recombinase. In one embodiment, the genetically engineered bacterium dies or is no longer viable when the first essential gene and the second essential gene are both excised. In one embodiment, the genetically engineered bacterium dies or is no longer viable when either the first essential gene is excised or the second essential gene is excised by the first recombinase.

In one embodiment, the first excision enzyme is Xis1. In one embodiment, the first excision enzyme is Xis2. In one embodiment, the first excision enzyme is Xis1, and the second excision enzyme is Xis2.

In one embodiment, the genetically engineered bacterium dies after the at least one recombination event occurs. In another embodiment, the genetically engineered bacterium is no longer viable after the at least one recombination event occurs.

In any of these embodiment, the recombinase can be a recombinase selected from the group consisting of: BxbI, PhiC31, TP901, BxbI, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34, or a biologically active fragment thereof.

In the above-described kill-switch circuits, a toxin is produced in the presence of an environmental factor or signal. In another aspect of kill-switch circuitry, a toxin may be repressed in the presence of an environmental factor (not produced) and then produced once the environmental condition or external signal is no longer present. Such kill switches are called repression-based kill switches and represent systems in which the bacterial cells are viable only in the presence of an external factor or signal, such as arabinose or other sugar. Exemplary kill switch designs in which the toxin is repressed in the presence of an external factor or signal (and activated once the external signal is removed) are described herein. The disclosure provides engineered bacterial cells which express one or more heterologous gene(s) upon sensing arabinose or other sugar in the exogenous environment. In this aspect, the engineered bacterial cells contain the araC gene, which encodes the AraC transcription factor, as well as one or more genes under the control of the araBAD promoter. In the absence of arabinose, the AraC transcription factor adopts a conformation that represses transcription of genes under the control of the araBAD promoter. In the presence of arabinose, the AraC transcription factor undergoes a conformational change that allows it to bind to and activate the araBAD promoter, which induces expression of the desired gene, for example tetR, which represses expression of a toxin gene. In this embodiment, the toxin gene is repressed in the presence of arabinose or other sugar. In an environment where arabinose is not present, the tetR gene is not activated and the toxin is expressed, thereby killing the bacteria. The arabinose system can also be used to express an essential gene, in which the essential gene is only expressed in the presence of arabinose or other sugar and is not expressed when arabinose or other sugar is absent from the environment.

Thus, in some embodiments in which one or more heterologous gene(s) are expressed upon sensing arabinose in the exogenous environment, the one or more heterologous genes are directly or indirectly under the control of the araBAD promoter. In some embodiments, the expressed heterologous gene is selected from one or more of the following: a heterologous therapeutic gene, a heterologous gene encoding an antitoxin, a heterologous gene encoding a repressor protein or polypeptide, for example, a TetR repressor, a heterologous gene encoding an essential protein not found in the bacterial cell, and/or a heterologous encoding a regulatory protein or polypeptide.

Arabinose inducible promoters are known in the art, including Pₐᵣₐ, P_{araB}, P_{araC}, and P_{araBAD}. In one embodiment, the arabinose inducible promoter is from E. coli. In some embodiments, the P_{araC} promoter and the P_{araBAD} promoter operate as a bidirectional promoter, with the P_{araBAD} promoter controlling expression of a heterologous gene(s) in one direction, and the P_{araC} (in close proximity to, and on the opposite strand from the P_{araBAD} promoter), controlling expression of a heterologous gene(s) in the other direction. In the presence of arabinose, transcription of both heterologous genes from both promoters is induced. However, in the absence of arabinose, transcription of both heterologous genes from both promoters is not induced.

In one exemplary embodiment of the disclosure, the engineered bacteria of the present disclosure contains a kill-switch having at least the following sequences: a P_{araBAD} promoter operably linked to a heterologous gene encoding a Tetracycline Repressor Protein (TetR), a P_{araC} promoter operably linked to a heterologous gene encoding AraC transcription factor, and a heterologous gene encoding a bacterial toxin operably linked to a promoter which is repressed by the Tetracycline Repressor Protein (P_{TetR}). In the presence of arabinose, the AraC transcription factor activates the P_{araBAD} promoter, which activates transcription of the TetR protein which, in turn, represses transcription of the toxin. In the absence of arabinose, however, AraC suppresses transcription from the P_{araBAD} promoter and no TetR protein is expressed. In this case, expression of the heterologous toxin gene is activated, and the toxin is expressed. The toxin builds up in the engineered bacterial cell, and the engineered bacterial cell is killed. In one embodiment, the araC gene encoding the AraC transcription factor is under the control of a constitutive promoter and is therefore constitutively expressed.

In one embodiment of the disclosure, the engineered bacterial cell further comprises an antitoxin under the control of a constitutive promoter. In this situation, in the presence of arabinose, the toxin is not expressed due to repression by TetR protein, and the antitoxin protein builds-up in the cell. However, in the absence of arabinose, TetR protein is not expressed, and expression of the toxin is induced. The toxin begins to build-up within the engineered bacterial cell. The engineered bacterial cell is no longer viable once the toxin protein is present at either equal or greater amounts than that of the anti-toxin protein in the cell, and the engineered bacterial cell will be killed by the toxin.

In another embodiment of the disclosure, the engineered bacterial cell further comprises an antitoxin under the control of the P_{araBAD} promoter. In this situation, in the presence of arabinose, TetR and the anti-toxin are expressed, the anti-toxin builds up in the cell, and the toxin is not expressed due to repression by TetR protein. However, in the absence of arabinose, both the TetR protein and the anti-toxin are not expressed, and expression of the toxin is induced. The toxin begins to build-up within the engineered bacterial cell. The engineered bacterial cell is no longer viable once the toxin protein is expressed, and the engineered bacterial cell will be killed by the toxin.

In another exemplary embodiment of the disclosure, the engineered bacteria of the present disclosure contains a kill-switch having at least the following sequences: a P_{araBAD} promoter operably linked to a heterologous gene encoding an essential polypeptide not found in the engineered bacterial cell (and required for survival), and a P_{araC} promoter operably linked to a heterologous gene encoding AraC transcription factor. In the presence of arabinose, the AraC transcription factor activates the P_{araBAD} promoter, which activates transcription of the heterologous gene encoding the essential polypeptide, allowing the engineered bacterial cell to survive. In the absence of arabinose, however, AraC suppresses transcription from the P_{araBAD} promoter and the essential protein required for survival is not expressed. In this case, the engineered bacterial cell dies in the absence of arabinose. In some embodiments, the sequence of P_{araBAD} promoter operably linked to a heterologous gene encoding an essential polypeptide not found in the engineered bacterial cell can be present in the bacterial cell in conjunction with the TetR/toxin kill-switch system described directly above. In some embodiments, the sequence of P_{araBAD} promoter operably linked to a heterologous gene encoding an essential polypeptide not found in the engineered bacterial cell can be present in the bacterial cell in conjunction with the TetR/toxin/anti-toxin kill-switch system described directly above.

In yet other embodiments, the bacteria may comprise a plasmid stability system with a plasmid that produces both a short-lived anti-toxin and a long-lived toxin. In this system, the bacterial cell produces equal amounts of toxin and anti-toxin to neutralize the toxin. However, if/when the cell loses the plasmid, the short-lived anti-toxin begins to decay. When the anti-toxin decays completely the cell dies as a result of the longer-lived toxin killing it.

In some embodiments, the engineered bacteria of the present disclosure, for example, bacteria described herein may further comprise the gene(s) encoding the components of any of the above-described kill-switch circuits.

In any of the above-described embodiments, the bacterial toxin is selected from the group consisting of a lysin, Hok, Fst, TisB, LdrD, Kid, SymE, MazF, FlmA, Ibs, XCV2162, dinJ, CcdB, MazF, ParE, YafO, Zeta, hicB, relB, yhaV, yoeB, chpBK, hipA, microcin B, microcin B17, microcin C, microcin C7-051, microcin J25, microcin ColV, microcin 24, microcin L, microcin D93, microcin L, microcin E492, microcin H47, microcin 147, microcin M, colicin A, colicin E1, colicin K, colicin N, colicin U, colicin B, colicin Ia, colicin Ib, colicin 5, colicin10, colicin S4, colicin Y, colicin E2, colicin E7, colicin E8, colicin E9, colicin E3, colicin E4, colicin E6; colicin E5, colicin D, colicin M, and cloacin DF13, or a biologically active fragment thereof.

In any of the above-described embodiments, the anti-toxin is selected from the group consisting of an anti-lysin, Sok, RNAII, IstR, RdlD, Kis, SymR, MazE, FlmB, Sib, ptaRNA1, yafQ, CcdA, MazE, ParD, yafN, Epsilon, HicA, relE, prlF, yefM, chpBI, hipB, MccE, MccE^{CTD}, MccF, Cai, ImmE1, Cki, Cni, Cui, Cbi, Iia, Imm, Cfi, Im10, Csi, Cyi, Im2, Im7, Im8, Im9, Im3, Im4, ImmE6, cloacin immunity protein (Cim), ImmE5, ImmD, and Cmi, or a biologically active fragment thereof.

In one embodiment, the bacterial toxin is bactericidal to the genetically engineered bacterium. In one embodiment, the bacterial toxin is bacteriostatic to the genetically engineered bacterium.

In one embodiment, the method further comprises administering a second engineered bacterial cell to the subject, wherein the second engineered bacterial cell comprises a heterologous reporter gene operably linked to an inducible promoter that is directly or indirectly induced by an exogenous environmental condition. In one embodiment, the heterologous reporter gene is a fluorescence gene. In one embodiment, the fluorescence gene encodes a green fluorescence protein (GFP). In another embodiment, the method further comprises administering a second engineered bacterial cell to the subject, wherein the second engineered bacterial cell expresses a lacZ reporter construct that cleaves a substrate to produce a small molecule that can be detected in urine (see, for example, Danio et al., Science Translational Medicine, 7(289):1-12, 2015, the entire contents of which are expressly incorporated herein by reference).

Isolated Plasmids

In other embodiments, the disclosure provides an isolated plasmid comprising a first nucleic acid encoding a first payload operably linked to a first inducible promoter, and a second nucleic acid encoding a second payload operably linked to a second inducible promoter. In other embodiments, the disclosure provides an isolated plasmid further comprising a third nucleic acid encoding a third payload operably linked to a third inducible promoter. In other embodiments, the disclosure provides a plasmid comprising four, five, six, or more nucleic acids encoding four, five, six, or more payloads operably linked to inducible promoters. In any of the embodiments described here, the first, second, third, fourth, fifth, sixth, “payload(s)” can be a propionate catabolism enzyme, a propionate transporter, a propionate binding protein, or other sequence described herein. In one embodiment, the nucleic acid encoding the first payload and the nucleic acid encoding the second payload are operably linked to the first inducible promoter. In one embodiment, the nucleic acid encoding the first payload is operably linked to a first inducible promoter and the nucleic acid encoding the second payload is operably linked to a second inducible promoter. In one embodiment, the first inducible promoter and the second inducible promoter are separate copies of the same inducible promoter. In another embodiment, the first inducible promoter and the second inducible promoter are different inducible promoters. In other embodiments comprising a third nucleic acid, the nucleic acid encoding the third payload and the nucleic acid encoding the first and second payloads are all operably linked to the same inducible promoter. In other embodiments, the nucleic acid encoding the first payload is operably linked to a first inducible promoter, the nucleic acid encoding the second payload is operably linked to a second inducible promoter, and the nucleic acid encoding third payload is operably linked to a third inducible promoter. In some embodiments, the first, second, and third inducible promoters are separate copies of the same inducible promoter. In other embodiments, the first inducible promoter, the second inducible promoter, and the third inducible promoter are different inducible promoters. In some embodiments, the first promoter, the second promoter, and the optional third promoter, or the first promoter and the second promoter and the optional third promoter, are each directly or indirectly induced by low-oxygen or anaerobic conditions. In other embodiments, the first promoter, the second promoter, and the optional third promoter, or the first promoter and the second promoter and the optional third promoter, are each a fumarate and nitrate reduction regulator (FNR) responsive promoter. In other embodiments, the first promoter, the second promoter, and the optional third promoter, or the first promoter and the second promoter and the optional third promoter are each a ROS-inducible regulatory region. In other embodiments, the first promoter, the second promoter, and the optional third promoter, or the first promoter and the second promoter and the optional third promoter are each a RNS-inducible regulatory region.

In some embodiments, the heterologous gene encoding a propionate catabolism enzyme is operably linked to a constitutive promoter. In one embodiment, the constitutive promoter is a lac promoter. In another embodiment, the constitutive promoter is a tet promoter. In another embodiment, the constitutive promoter is a constitutive Escherichia coli σ32 promoter. In another embodiment, the constitutive promoter is a constitutive Escherichia coli σ70 promoter. In another embodiment, the constitutive promoter is a constitutive Bacillus subtilis A promoter. In another embodiment, the constitutive promoter is a constitutive Bacillus subtilis σB promoter. In another embodiment, the constitutive promoter is a Salmonella promoter. In other embodiments, the constitutive promoter is a bacteriophage T7 promoter. In other embodiments, the constitutive promoter is and a bacteriophage SP6 promoter. In any of the above-described embodiments, the plasmid further comprises a heterologous gene encoding a propionate transporter, a propionate binding protein, and/or a kill switch construct, which may be operably linked to a constitutive promoter or an inducible promoter.

In some embodiments, the isolated plasmid comprises at least one heterologous propionate catabolism enzyme gene operably linked to a first inducible promoter; a heterologous gene encoding a TetR protein operably linked to a ParaBAD promoter, a heterologous gene encoding AraC operably linked to a ParaC promoter, a heterologous gene encoding an antitoxin operably linked to a constitutive promoter, and a heterologous gene encoding a toxin operably linked to a PTetR promoter. In another embodiment, the isolated plasmid comprises at least one heterologous gene encoding a propionate catabolism enzyme operably linked to a first inducible promoter; a heterologous gene encoding a TetR protein and an anti-toxin operably linked to a ParaBAD promoter, a heterologous gene encoding AraC operably linked to a ParaC promoter, and a heterologous gene encoding a toxin operably linked to a PTetR promoter.

In some embodiments, a first nucleic acid encoding a propionate catabolism enzyme comprises a prpE and/or a Pha gene. In other embodiments, a first nucleic acid encoding a propionate catabolism enzyme is a Pha gene or a Pha operon, e.g. prpE-phaB-phaC-phaA. In some embodiments, the prpE gene or Pha gene or Pha operon is coexpressed with an additional propionate catabolism gene or gene cassette, e.g. a MMCA cassette and/or a 2MC cassette described herein. In other embodiments, a gene encoding a succinate exporter, e.g., SucE1 and/or DcuC, is further expressed. In other embodiments, a propionate importer is further expressed.

In some embodiments, a first nucleic acid encoding a propionate catabolism enzyme comprises a prpE and/or a MMCA pathway gene. In other embodiments, a first nucleic acid encoding a propionate catabolism enzyme is a prpE and/or a MMCA pathway gene or a MMCA pathway operon, e.g. prpE-accA1-pccB-mmcE-mutA-mutB or prpE-accA1-pccB or mmcE-mutA-mutB. In some embodiments, the prpE and/or a MMCA pathway gene or a MMCA pathway operon is coexpressed with an additional propionate catabolism gene or gene cassette, e.g. a Pha cassette and/or a 2MC cassette described herein. In other embodiments, a gene encoding a succinate exporter, e.g., SucE1 and/or DcuC, is further expressed. In other embodiments, a propionate importer is further expressed.

In some embodiments, a first nucleic acid encoding a propionate catabolism enzyme comprises a prpE and/or a 2MC pathway gene. In other embodiments, a first nucleic acid encoding a propionate catabolism enzyme is a prpE and/or a 2MC pathway gene or a 2MC pathway operon, e.g. prpB-prpC-prpD-prpE or prpB-prpC-prpD. In some embodiments, the prpE and/or a 2MC pathway gene or a 2MC pathway operon is coexpressed with an additional propionate catabolism gene or gene cassette, e.g. a Pha cassette and/or a MMCA cassette described herein. In other embodiments, a gene encoding a succinate exporter, e.g., SucE1 and/or DcuC, is further expressed. In other embodiments, a propionate importer is further expressed.

In one embodiment, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

In one embodiment, the bacterial cell further comprises a genetic mutation in an endogenous gene encoding a lysine acetyltransferase, e.g. pka, which propionylates and inactivates prpE. In another embodiment, the bacterial cell further comprises a genetic mutation which reduces export of propionate and/or its metabolites from the bacterial cell.

In one embodiment, the bacterial cell further comprises a genetic mutation in an endogenous gene encoding a propionate biosynthesis gene, wherein the genetic mutation reduces biosynthesis of propionate and one or more of its metabolites in the bacterial cell.

Multiple Mechanisms of Action

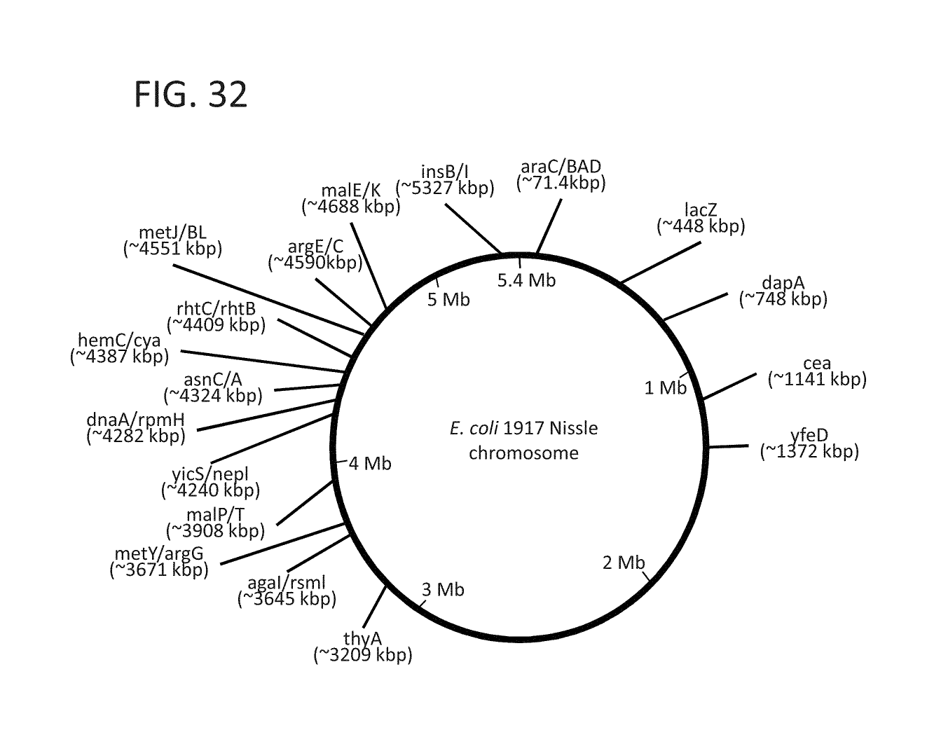

In some embodiments, the bacteria are genetically engineered to include multiple mechanisms of action (MOAs), e.g., circuits producing multiple copies of the same product (e.g., to enhance copy number) or circuits performing multiple different functions. Examples of insertion sites include, but are not limited to, malE/K, insB/I, araC/BAD, lacZ, dapA, cea, and other shown in FIG. 32. For example, the genetically engineered bacteria may include four copies of a propionate catabolism gene or propionate catabolism gene cassette, or four copies of a propionate catabolism gene inserted at four different insertion sites, e.g., malE/K, insB/I, araC/BAD, and lacZ. Alternatively, the genetically engineered bacteria may include one or more copies of a propionate catabolism gene or gene cassette inserted at one or more different insertion sites, e.g., malE/K, insB/I, and lacZ, one or more copies of a propionate catabolism gene or gene cassette inserted at one or more different insertion sites, e.g., dapA, cea, and araC/BAD and/or one or more copies of a propionate catabolism gene or gene cassette inserted at one or more different insertion sites.

In some embodiments, the genetically engineered bacteria comprise one or more of: (1) one or more gene(s) and/or gene cassettes encoding one or more propionate catabolism enzyme(s), in wild type or in a mutated form (for increased stability or metabolic activity); (2) one or more gene(s) and/or gene cassette(s) encoding one or more transporter(s) for uptake of propionate and/or one or more of its metabolites, including methylmalonic acid, in wild type or in mutated form (for increased stability or metabolic activity); (3) one or more gene(s) or gene cassette(s) encoding one or more propionate catabolism enzyme(s) for secretion and extracellular degradation of propionate and/or one or more of its metabolites, (4) one or more gene(s) or gene cassette(s) encoding one or more components of secretion machinery, as described herein (5) one or more auxotrophies, e.g., deltaThyA; (6) one or more gene(s) or gene cassette(s) encoding one or more antibiotic resistance(s), including but not limited to, kanamycin or chloramphenicol resistance; (7) one or more modifications that increase succinate export from the bacterial cell; (8) one or modifications that reduce succinate import into the bacterial cell; (9) mutations/deletions in genes, as described herein, e.g., pka, succinate importers or propionate exporters (10) mutations/deletions in genes of the endogenous propionate synthesis pathway (11) one or more gene(s) and/or gene cassettes encoding one or more ammonium consuming circuit(s) and optionally one or more gene(s) encoding ammonium transporter(s)/importer(s) and optionally one or more gene(s) encoding one or more arginine exporter(s), as described in co-owned U.S. Pat. No. 9,487,764 and US Patent Publication No. US20160177274, the contents of each of which is herein incorporated by reference in their entireties. (12) one or more gene(s) and or gene cassette(s) for the catabolism of branched chain amino acids (BCAA) (e.g., leucine, isoleucine, and/or valine), and optionally one or more BCAA transporter(s)/importer(s) and metabolite exporter(s) as described in co-owned International Patent Application No. PCT/US2016/037098, the contents of which is herein incorporated by reference in its entirety. In some embodiments, the genetically engineered bacteria comprise two or more different pathway cassettes or operons comprising propionate catabolism enzymes. In some embodiments, the genetically engineered bacteria comprise one or more gene(s) or gene cassette(s) encoding one or more propionate catabolism enzymes. In some embodiments, the genetically engineered bacteria comprise gene sequence(s) encoding one or more propionate catabolism enzymes selected from PrpE, AccA1, PccB, MmcE, MutA, and MutB, and combinations thereof. In some embodiments, the genetically engineered bacteria comprise gene sequence(s) comprising two or more copies of any genes selected from prpE, accA1, pccB, mmcE, mutA, and mutB. In some embodiments, the genetically engineered bacteria comprise gene sequence encoding one or more propionate catabolism enzymes selected from PrpE, PhaB, PhaC, and PhaA, and combinations thereof. In some embodiments, the genetically engineered bacteria comprise gene sequence(s) comprising two or more copies of any genes selected from prpE, phaB, phaC, and phaA. In some embodiments, the genetically engineered bacteria comprise gene sequence encoding one or more propionate catabolism enzymes selected from PrpB, PrpC, PrpD, and PrpE, and combinations thereof. In some embodiments, the genetically engineered bacteria comprise gene sequence(s) comprising two or more copies of any genes selected from prpB-prpC, prpD, and prpE. Non-limiting examples of combinations include genetically engineered bacteria comprising one or more MMCA pathway operon(s) (e.g., prpE-accA1-pccB-mmcE-mutA-mutB, or prpE-accA1-pccB and mmcE-mutA-mutB) in combination with one or more PHA pathway operon(s) (e.g., prpE-phaB-phaC-phaA). In another non-limiting example of combinations, the genetically engineered bacteria comprise one or more MMCA pathway operon(s) (e.g., prpE-accA1-pccB-mmcE-mutA-mutB, or prpE-accA1-pccB and mmcE-mutA-mutB) in combination with one or more 2MC pathway operon(s) (e.g., prpB-prpC-prpD-prpE). In another non-limiting example of combinations, the genetically engineered bacteria comprise one or more MMCA pathway operon(s) (e.g., prpE-accA1-pccB-mmcE-mutA-mutB, or prpE-accA1-pccB and mmcE-mutA-mutB), one or more 2MC pathway operon(s) (e.g., prpB-prpC-prpD-prpE), and one or more PHA pathway operon(s) (e.g., prpE-phaB-phaC-phaA). In another non-limiting example of combinations, the genetically engineered bacteria comprise one or more 2MC pathway operon(s) (e.g., prpB-prpC-prpD-prpE), and one or more PHA pathway operon(s) (e.g., prpE-phaB-phaC-phaA). In another non-limiting example of combinations, the genetically engineered bacteria comprise one or more 2MC pathway operon(s) (e.g., prpB-prpC-prpD-prpE), and one or more MMCA pathway operon(s) (e.g., prpE-accA1-pccB-mmcE-mutA-mutB, or prpE-accA1-pccB and mmcE-mutA-mutB).

Non-limiting examples of combinations include genetically engineered bacteria comprising one or more MMCA pathway operon(s) (e.g., prpE-accA1-pccB-mmcE-mutA-mutB, or prpE-accA1-pccB and mmcE-mutA-mutB) in combination with one or more PHA pathway operon(s) (e.g., prpE-phaB-phaC-phaA) and in combination with one or more cassettes comprising matB. In another non-limiting example of combinations, the genetically engineered bacteria comprise one or more MMCA pathway operon(s) (e.g., prpE-accA1-pccB-mmcE-mutA-mutB, or prpE-accA1-pccB and mmcE-mutA-mutB) in combination with one or more 2MC pathway operon(s) (e.g., prpB-prpC-prpD-prpE) and in combination with one or more cassettes comprising matB. In another non-limiting example of combinations, the genetically engineered bacteria comprise one or more MMCA pathway operon(s) (e.g., prpE-accA1-pccB-mmcE-mutA-mutB, or prpE-accA1-pccB and mmcE-mutA-mutB), one or more 2MC pathway operon(s) (e.g., prpB-prpC-prpD-prpE), and one or more PHA pathway operon(s) (e.g., prpE-phaB-phaC-phaA) and in combination with one or more cassettes comprising matB. In another non-limiting example of combinations, the genetically engineered bacteria comprise one or more 2MC pathway operon(s) (e.g., prpB-prpC-prpD-prpE), and one or more PHA pathway operon(s) (e.g., prpE-phaB-phaC-phaA) and in combination with one or more cassettes comprising MatB. In another non-limiting example of combinations, the genetically engineered bacteria comprise one or more 2MC pathway operon(s) (e.g., prpB-prpC-prpD-prpE), and one or more MMCA pathway operon(s) (e.g., prpE-accA1-pccB-mmcE-mutA-mutB, or prpE-accA1-pccB and mmcE-mutA-mutB) and in combination with one or more cassettes comprising matB. Any of the combinations described above comprising matB may or may not comprise prpE, e.g., may comprise matB in lieu of prpE.

In some embodiments, the genetically engineered bacteria comprise one or more gene(s) or gene cassette(s) encoding one or more propionate catabolism enzymes and one or more gene(s) or gene cassette(s) encoding one or more propionate transporters (importers), such as any of the propionate transporters described herein and otherwise known in the art.

In some embodiments, the genetically engineered bacteria comprise one or more gene(s) or gene cassette(s) encoding one or more propionate catabolism enzymes and one or more gene(s) or gene cassette(s) encoding one or more succinate exporters, e.g. SucE1 and/or dcuC. Non-limiting examples of combinations include genetically engineered bacteria comprising one or more MMCA pathway operon(s) (e.g., prpE-accA1-pccB-mmcE-mutA-mutB, or prpE-accA1-pccB and mmcE-mutA-mutB) in combination with one or more PHA pathway operon(s) (e.g., prpE-phaB-phaC-phaA) and one or more gene(s) or gene cassette(s) encoding one or more succinate exporters, e.g. SucE1 and/or dcuC. In another non-limiting example of combinations, the genetically engineered bacteria comprise one or more MMCA pathway operon(s) (e.g., prpE-accA1-pccB-mmcE-mutA-mutB, or prpE-accA1-pccB and mmcE-mutA-mutB) in combination with one or more 2MC pathway operon(s) (e.g., prpB-prpC-prpD-prpE) and one or more gene(s) or gene cassette(s) encoding one or more succinate exporters, e.g. SucE1 and/or dcuC. In another non-limiting example of combinations, the genetically engineered bacteria comprise one or more MMCA pathway operon(s) (e.g., prpE-accA1-pccB-mmcE-mutA-mutB, or prpE-accA1-pccB and mmcE-mutA-mutB), one or more 2MC pathway operon(s) (e.g., prpB-prpC-prpD-prpE), and one or more PHA pathway operon(s) (e.g., prpE-phaB-phaC-phaA) and one or more gene(s) or gene cassette(s) encoding one or more succinate exporters, e.g. SucE1 and/or dcuC. In another non-limiting example of combinations, the genetically engineered bacteria comprise one or more 2MC pathway operon(s) (e.g., prpB-prpC-prpD-prpE), and one or more PHA pathway operon(s) (e.g., prpE-phaB-phaC-phaA) and one or more gene(s) or gene cassette(s) encoding one or more succinate exporters, e.g. SucE1 and/or dcuC. In another non-limiting example of combinations, the genetically engineered bacteria comprise one or more 2MC pathway operon(s) (e.g., prpB-prpC-prpD-prpE), and one or more MMCA pathway operon(s) (e.g., prpE-accA1-pccB-mmcE-mutA-mutB, or prpE-accA1-pccB and mmcE-mutA-mutB) and one or more gene(s) or gene cassette(s) encoding one or more succinate exporters, e.g. SucE1 and/or dcuC. In other non-limiting examples, the genetically engineered bacteria comprising one or more gene(s) or gene cassette(s) encoding one or more propionate catabolism enzymes and one or more gene(s) or gene cassette(s) encoding one or more succinate exporters, e.g. SucE1 and/or dcuC, e.g., as described supra, may comprise one or more gene(s) or gene cassette(s) comprising matB or matB may be substituted in lieu of prpE. In any of the embodiments, the engineered bacterium may also comprise gene sequence(s) encoding one or more propionate transporters.

In some embodiments, the genetically engineered bacteria comprise one or more gene(s) or gene cassette(s) encoding one or more propionate catabolism enzymes and one or more genetic modifications that reduce or decrease succinate import into the bacterial cell, such as any of the genetic modifications described herein and otherwise known in the art. The engineered bacterium may further comprise gene sequence(s) encoding one or more propionate transporters. The engineered bacterium may further comprise gene sequence encoding one or more succinate exporters. Thus, in some embodiments the engineered bacterium comprises one or more gene(s) or gene cassette(s) encoding one or more propionate catabolism enzymes, one or more genetic modifications that reduce or decrease succinate import into the bacterial cell, and gene sequence(s) encoding one or more propionate transporters. In some embodiments, the engineered bacterium comprises one or more gene(s) or gene cassette(s) encoding one or more propionate catabolism enzymes, one or more genetic modifications that reduce or decrease succinate import into the bacterial cell, and gene sequence(s) encoding one or more succinate exporters. In some embodiments, the engineered bacterium comprises one or more gene(s) or gene cassette(s) encoding one or more propionate catabolism enzymes, one or more genetic modifications that reduce or decrease succinate import into the bacterial cell, gene sequence(s) encoding one or more propionate transporters, and gene sequence(s) encoding one or more succinate exporters.

In some embodiments, certain catalytic steps are rate limiting and in such a case it may be beneficial to add additional copies of one or more gene(s) encoding one or more rate limiting enzyme(s). In a non-limiting example, the genetically engineered bacteria may encode one or more PHA pathway operon(s) (e.g., prpE-phaB-phaC-phaA) and one or more additional gene(s) or gene cassette(s) encoding one or more of phaA. In a non-limiting example, the genetically engineered bacteria may one or more PHA pathway operon(s) (e.g., prpE-phaB-phaC-phaA) and one or more additional gene(s) or gene cassette(s) encoding one or more of prpE and/or phaB and/or phaC and/or phaA.

In a non-limiting example, the genetically engineered bacteria may encode one or more MMCA pathway operon(s) e.g., prpE-accA1-pccB-mmcE-mutA-mutB, or prpE-accA1-pccB and mmcE-mutA-mutB) and one or more additional gene(s) or gene cassette(s) encoding one or more of prpE and/or accA1 and/or pccB and/or mmcE and/or mutA and/or mutB. In another non-limiting example, the genetically engineered bacteria may one or more 2MC pathway operon(s) (e.g., prpB-prpC-prpD-prpE) and one or more additional gene(s) or gene cassette(s) encoding prpB and/or prpC and/or prpD and/or prpE).

In some embodiments, each gene from a propionate catabolism pathway described herein, e.g., PHA, MMCA, and/or 2MC, can be expressed individually, each under control of a separate (same or different) promoter. For example, one or more of prpE and/or phaB and/or phaC and/or phaA can be expressed individually, each under control of a separate (same or different) promoter. For example, one or more of prpE and/or accA1 and/or pccB and/or mmcE and/or mutA and/or mutB can be expressed individually, each under control of a separate (same or different) promoter. For example, one or more of prpB and/or prpC and/or prpD and/or prpE can be expressed individually, each under control of a separate (same or different) promoter. In some embodiments, each gene from a propionate catabolism pathway described herein, e.g., a matB comprising pathway (e.g., matA, mmcE, mutA and mutB, and/or MatB, Acc1A, and PccB, (e.g., with PrpE)) can be expressed individually, each under control of a separate (same or different) promoter.

In certain embodiments the order of the genes within a gene cassette can be modified, e.g., to increase or decrease levels of a particular gene within a cassette. In a non-limiting example, the genetically engineered bacteria may encode one or more PHA pathway operon(s) (e.g., prpE-phaB-phaC-phaA), in phaC comes first or phaB comes first, or prpE comes first or phaA comes first. In a non-limiting example, the genetically engineered bacteria may encode one or more PHA pathway operon(s) (e.g., prpE-phaB-phaC-phaA), in which that phaC comes second or phaB comes second, or prpE comes second or phaA comes second. In a non-limiting example, the genetically engineered bacteria may encode one or more PHA pathway operon(s) (e.g., prpE-phaB-phaC-phaA), in which phaC comes third or phaB comes third, or prpE comes third or phaA comes third.

In a non-limiting example, the genetically engineered bacteria may encode one or more 2MC pathway operon(s) (e.g., prpB-prpC-prpD-prpE), in which prpB comes first or prpC comes first or prpD comes first or prpE comes first. In a non-limiting example, the genetically engineered bacteria may encode one or more 2MC pathway operon(s) (e.g., prpB-prpC-prpD-prpE), in which prpB comes second or prpC comes second or prpD comes second or prpE comes second. In a non-limiting example, the genetically engineered bacteria may encode one or more 2MC pathway operon(s) (e.g., prpB-prpC-prpD-prpE), in which prpB comes third or prpC comes third or prpD comes third or prpE comes third. In a non-limiting example, the genetically engineered bacteria may encode one or more 2MC pathway operon(s) (e.g., prpB-prpC-prpD-prpE), in which prpB comes fourth or prpC comes fourth or prpD comes fourth or prpE comes fourth.

In a non-limiting example, the genetically engineered bacteria may encode one or more MMCA operon(s) (e.g., prpE-accA1-pccB-mmcE-mutA-mutB, or prpE-accA1-pccB and mmcE-mutA-mutB) in which prpE comes first or accA1 comes first or pccB comes first or mmcE comes first or mutA comes first or mutB comes first. In a non-limiting example, the genetically engineered bacteria may encode one or more MMCA operon(s) (e.g., prpE-accA1-pccB-mmcE-mutA-mutB, or prpE-accA1-pccB and mmcE-mutA-mutB) in which prpE comes second or accA1 comes second or pccB comes second or mmcE comes second or mutA comes second or mutB comes second. In a non-limiting example, the genetically engineered bacteria may encode one or more MMCA operon(s) (e.g., prpE-accA1-pccB-mmcE-mutA-mutB, or prpE-accA1-pccB and mmcE-mutA-mutB) in which prpE comes third or accA1 comes third or pccB comes third or mmcE comes third or mutA comes third or mutB comes third. In a non-limiting example, the genetically engineered bacteria may encode one or more MMCA operon(s) (e.g., prpE-accA1-pccB-mmcE-mutA-mutB, or prpE-accA1-pccB and mmcE-mutA-mutB) in which prpE comes fourth, fifth or sixth or accA1 comes fourth, fifth or sixth or pccB comes fourth, fifth or sixth or mmcE comes fourth, fifth or sixth or mutA comes fourth, fifth or sixth or mutB comes fourth, fifth or sixth. In some embodiments, matB comes first, second, third, fourth, fifth, or sixth in a gene cassette comprising matB.

In any of the embodiments described in this section or elsewhere in the specification, any one or more the genes can be operably linked to a directly or indirectly inducible promoter, such as any of the promoters described herein, e.g., induced by low oxygen or anaerobic conditions, such as those found in the mammalian gut.

In certain embodiments, ribosome binding sites, e.g., stronger or weaker ribosome binding sites can be used to modulate (increase or decrease) the levels of expression of a propionate catabolism enzyme within a cassette.

In some embodiments, the genetically engineered bacteria further comprise mutations or deletions, e.g., in pka, succinate importers or propionate exporters, and an auxotrophy.

Host-Plasmid Mutual Dependency

In some embodiments, the genetically engineered bacteria also comprise a plasmid that has been modified to create a host-plasmid mutual dependency. In certain embodiments, the mutually dependent host-plasmid platform is GeneGuard (Wright et al., 2015). In some embodiments, the GeneGuard plasmid comprises (i) a conditional origin of replication, in which the requisite replication initiator protein is provided in trans; (ii) an auxotrophic modification that is rescued by the host via genomic translocation and is also compatible for use in rich media; and/or (iii) a nucleic acid sequence which encodes a broad-spectrum toxin. The toxin gene may be used to select against plasmid spread by making the plasmid DNA itself disadvantageous for strains not expressing the anti-toxin (e.g., a wild-type bacterium). In some embodiments, the GeneGuard plasmid is stable for at least one-hundred generations without antibiotic selection. In some embodiments, the GeneGuard plasmid does not disrupt growth of the host. The GeneGuard plasmid is used to greatly reduce unintentional plasmid propagation in the genetically engineered bacteria described herein.

The mutually dependent host-plasmid platform may be used alone or in combination with other biosafety mechanisms, such as those described herein (e.g., kill switches, auxotrophies). In some embodiments, the genetically engineered bacteria comprise a GeneGuard plasmid. In other embodiments, the genetically engineered bacteria comprise a GeneGuard plasmid and/or one or more kill switches. In other embodiments, the genetically engineered bacteria comprise a GeneGuard plasmid and/or one or more auxotrophies. In still other embodiments, the genetically engineered bacteria comprise a GeneGuard plasmid, one or more kill switches, and/or one or more auxotrophies.

In some embodiments, the vector comprises a conditional origin of replication. In some embodiments, the conditional origin of replication is a R6K or ColE2-P9. In embodiments where the plasmid comprises the conditional origin of replication R6K, the host cell expresses the replication initiator protein 7E. In embodiments where the plasmid comprises the conditional origin or replication ColE2, the host cell expresses the replication initiator protein RepA. It is understood by those of skill in the art that the expression of the replication initiator protein may be regulated so that a desired expression level of the protein is achieved in the host cell to thereby control the replication of the plasmid. For example, in some embodiments, the expression of the gene encoding the replication initiator protein may be placed under the control of a strong, moderate, or weak promoter to regulate the expression of the protein.

In some embodiments, the vector comprises a gene encoding a protein required for complementation of a host cell auxotrophy, preferably a rich-media compatible auxotrophy. In some embodiments, the host cell is auxotrophic for thymidine (ΔthyA), and the vector comprises the thymidylate synthase (thyA) gene. In some embodiments, the host cell is auxotrophic for diaminopimelic acid (ΔdapA) and the vector comprises the 4-hydroxy-tetrahydrodipicolinate synthase (dapA) gene. It is understood by those of skill in the art that the expression of the gene encoding a protein required for complementation of the host cell auxotrophy may be regulated so that a desired expression level of the protein is achieved in the host cell.

In some embodiments, the vector comprises a toxin gene. In some embodiments, the host cell comprises an anti-toxin gene encoding and/or required for the expression of an anti-toxin. In some embodiments, the toxin is Zeta and the anti-toxin is Epsilon. In some embodiments, the toxin is Kid, and the anti-toxin is Kis. In preferred embodiments, the toxin is bacteriostatic. Any of the toxin/antitoxin pairs described herein may be used in the vector systems of the present disclosure. It is understood by those of skill in the art that the expression of the gene encoding the toxin may be regulated using art known methods to prevent the expression levels of the toxin from being deleterious to a host cell that expresses the anti-toxin. For example, in some embodiments, the gene encoding the toxin may be regulated by a moderate promoter. In other embodiments, the gene encoding the toxin may be cloned adjacent to ribosomal binding site of interest to regulate the expression of the gene at desired levels (see, e.g., Wright et al. (2015)).

Integration

In some embodiments, any of the gene(s) or gene cassette(s) of the present disclosure may be integrated into the bacterial chromosome at one or more integration sites. One or more copies of the heterologous gene or heterologous gene cassette may be integrated into the bacterial chromosome. Having multiple copies of the gene or gene cassette integrated into the chromosome allows for greater production of the corresponding protein(s) and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

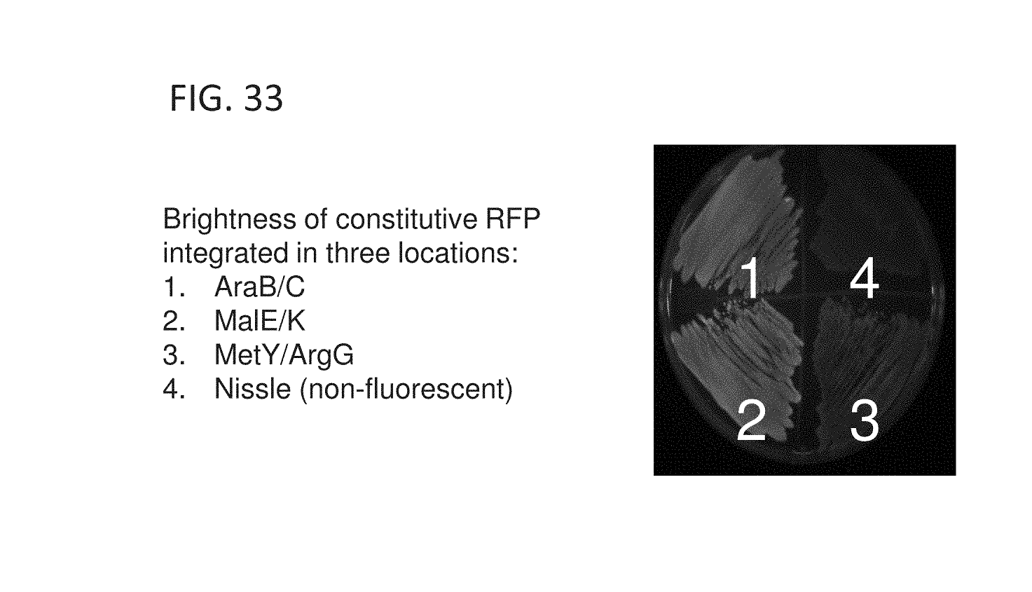

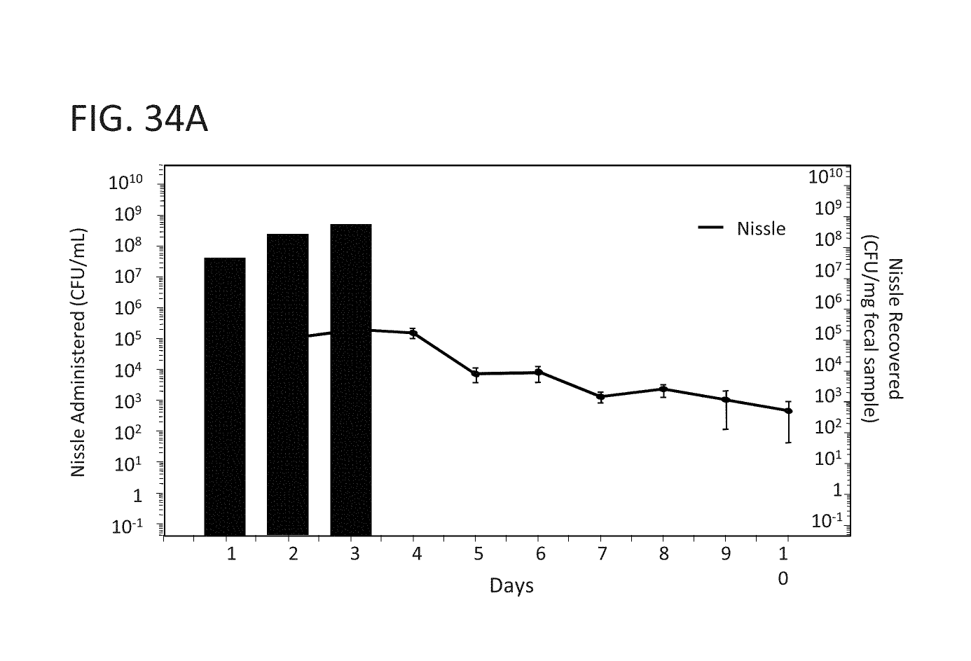

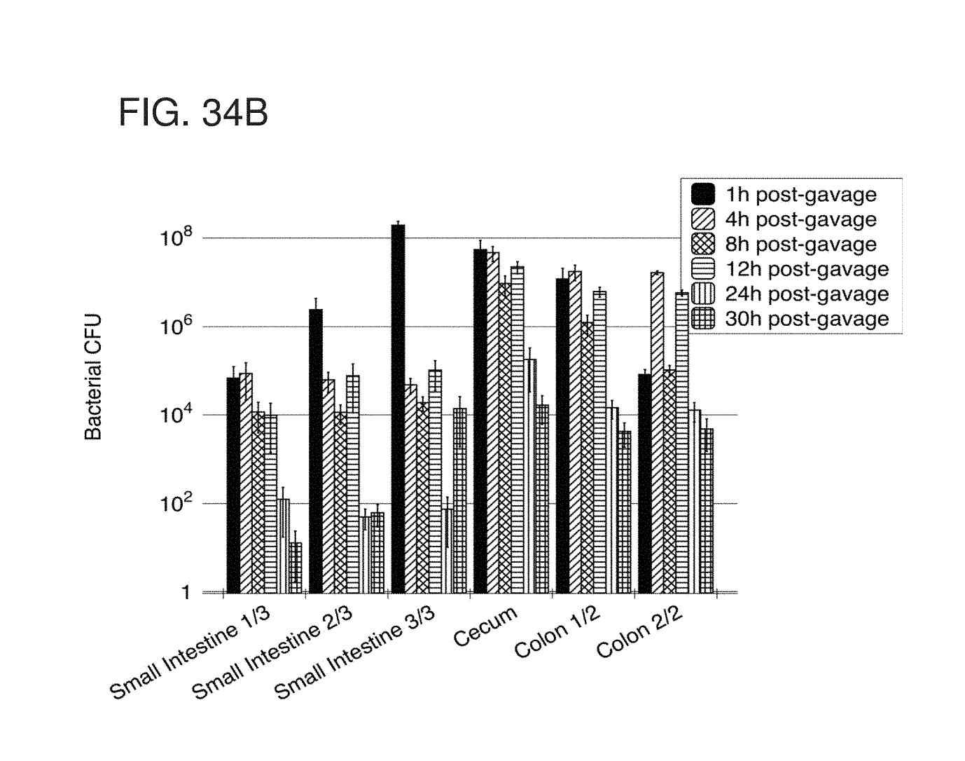

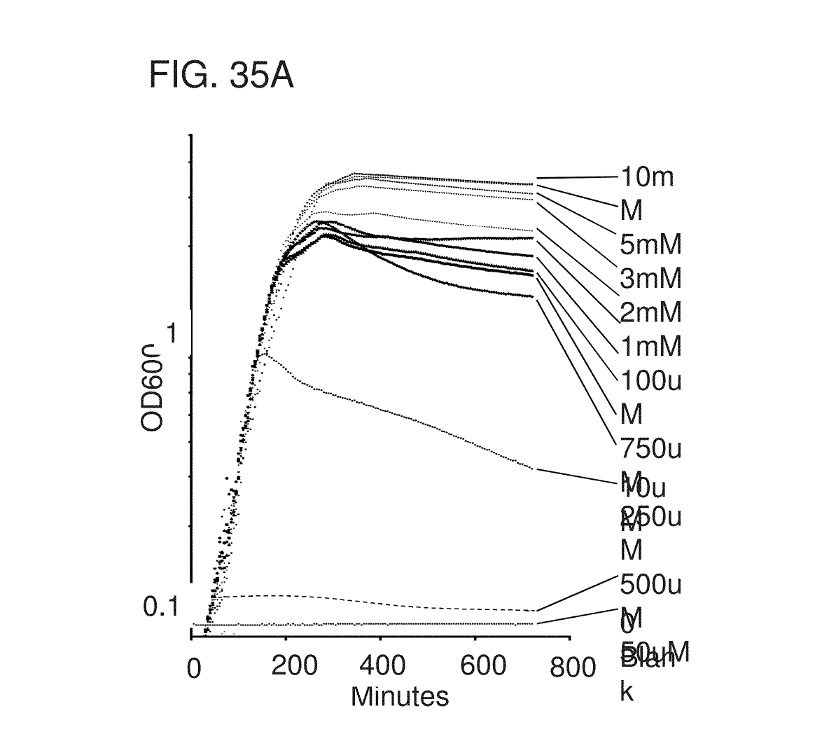

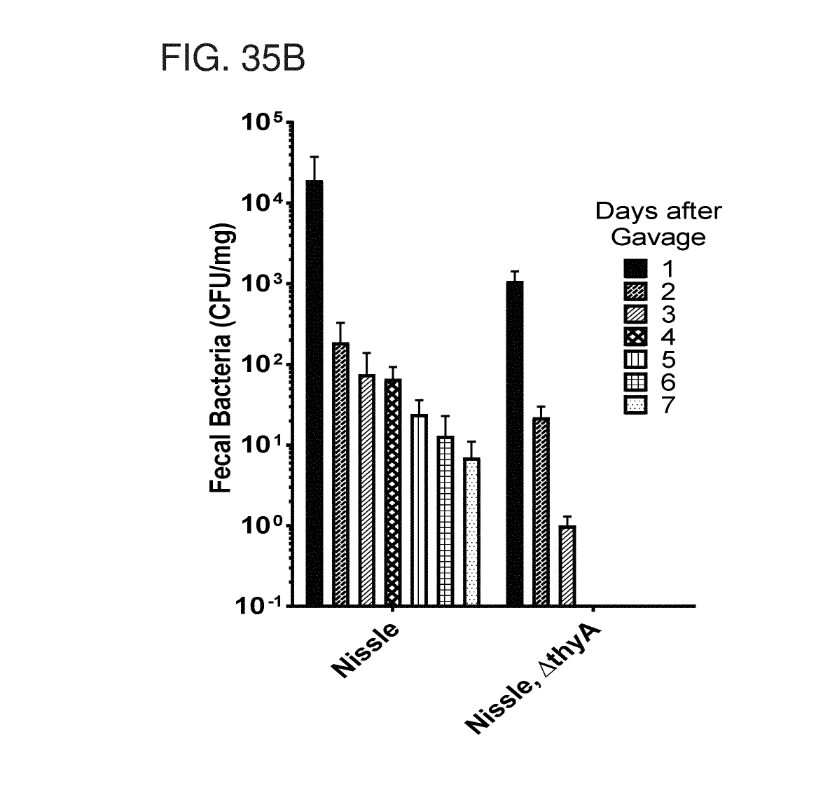

For example, FIG. 32 depicts a map of integration sites within the E. coli Nissle chromosome. FIG. 33 depicts three bacterial strains wherein the RFP gene has been successfully integrated into the bacterial chromosome at an integration site.

Secretion

In any of the embodiments described herein, in which the genetically engineered bacterium produces a propionate catabolism enzyme to be secreted from the bacterium, the engineered bacterium may comprise a secretion mechanism and corresponding gene sequence(s) encoding the secretion system.

In some embodiments, the genetically engineered bacteria further comprise a native secretion mechanism or non-native secretion mechanism that is capable of secreting the propionate catabolism enzyme from the bacterial cytoplasm into the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

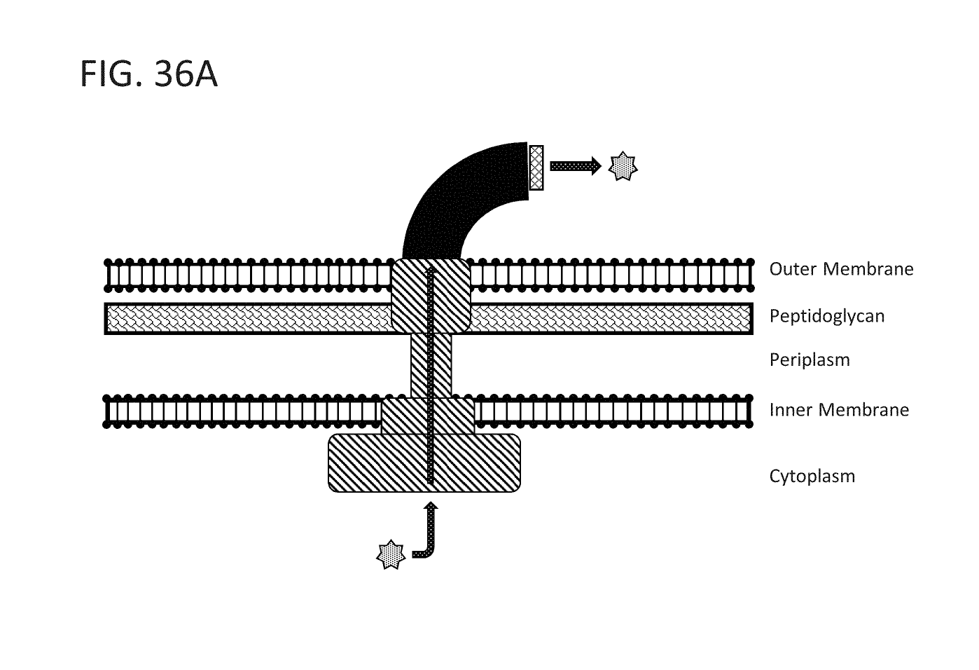

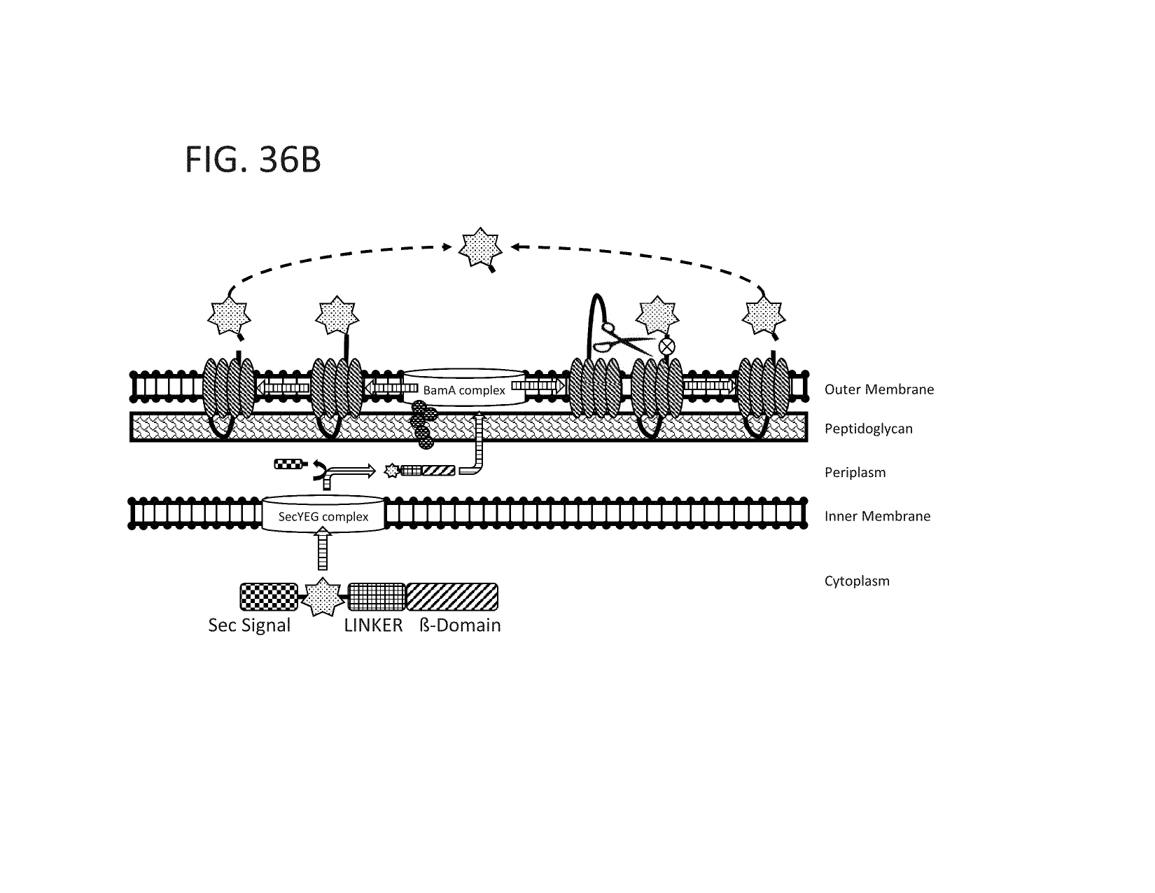

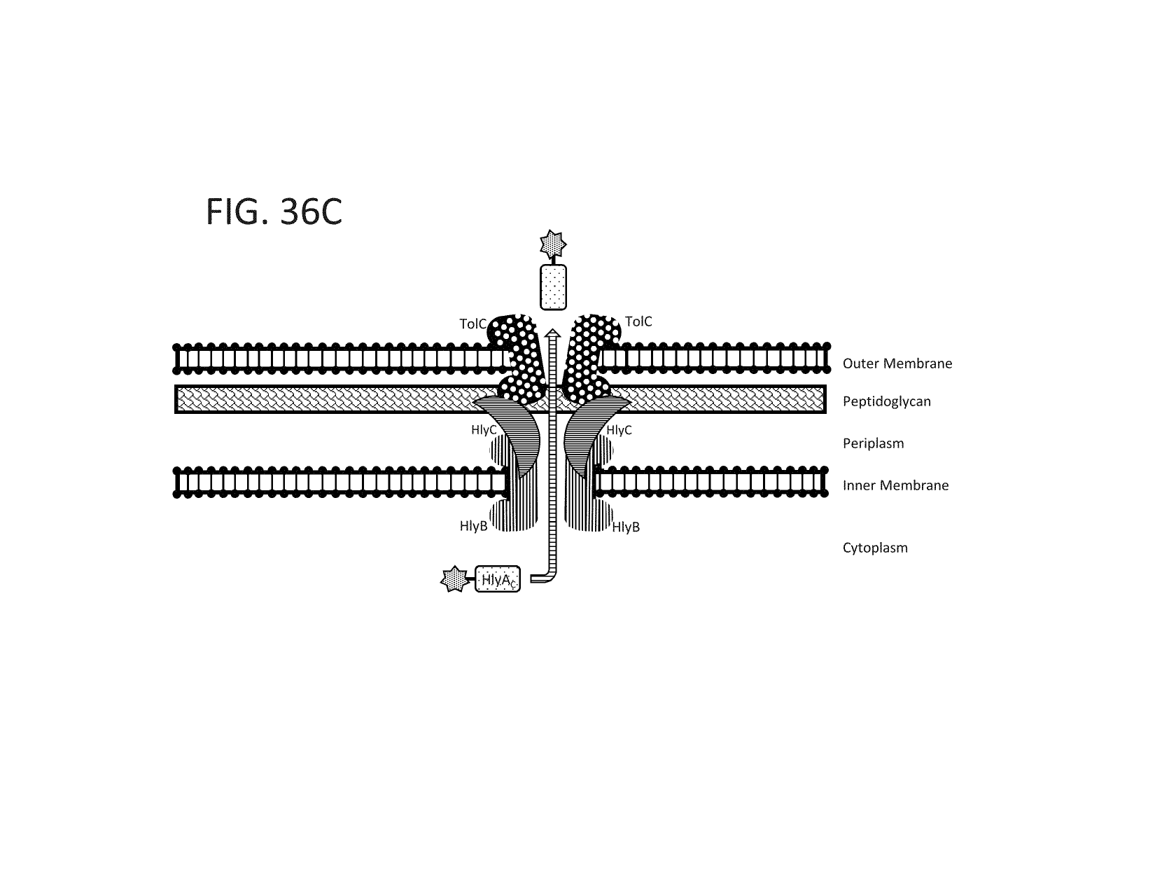

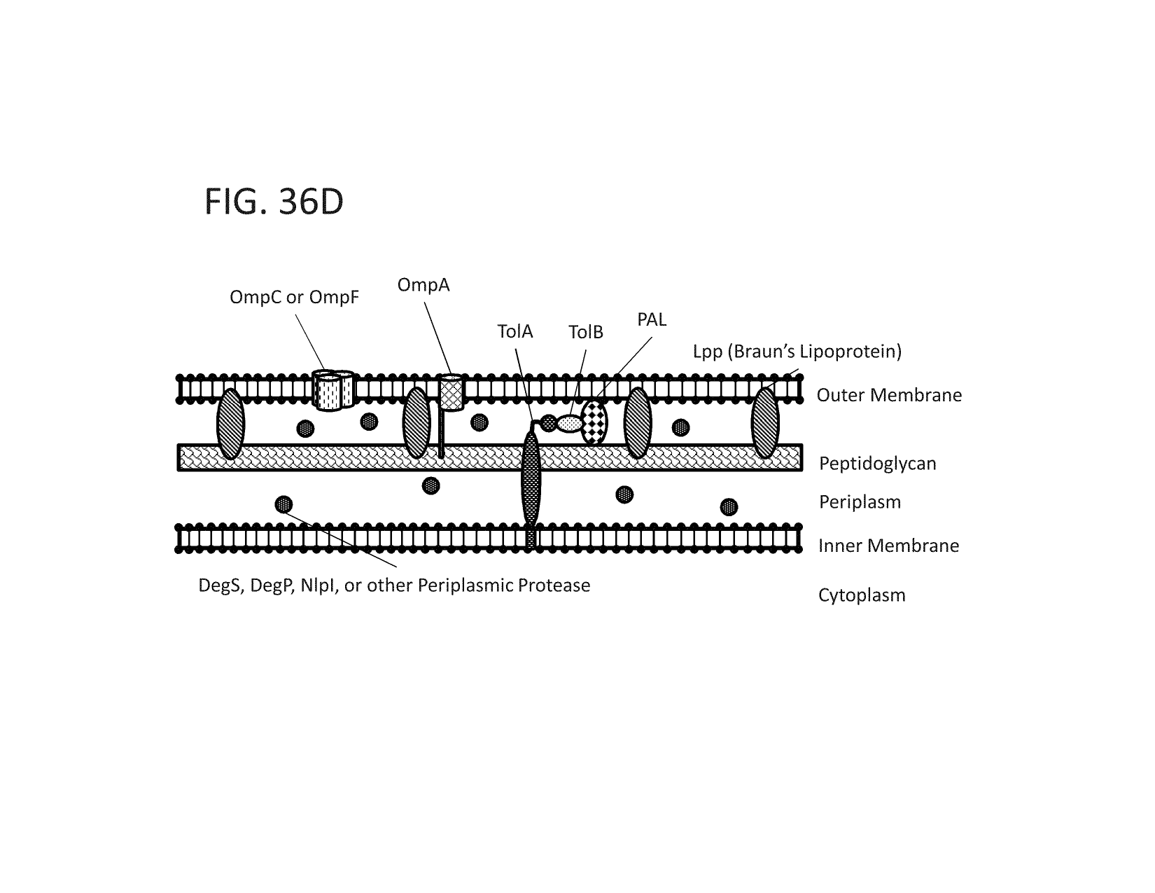

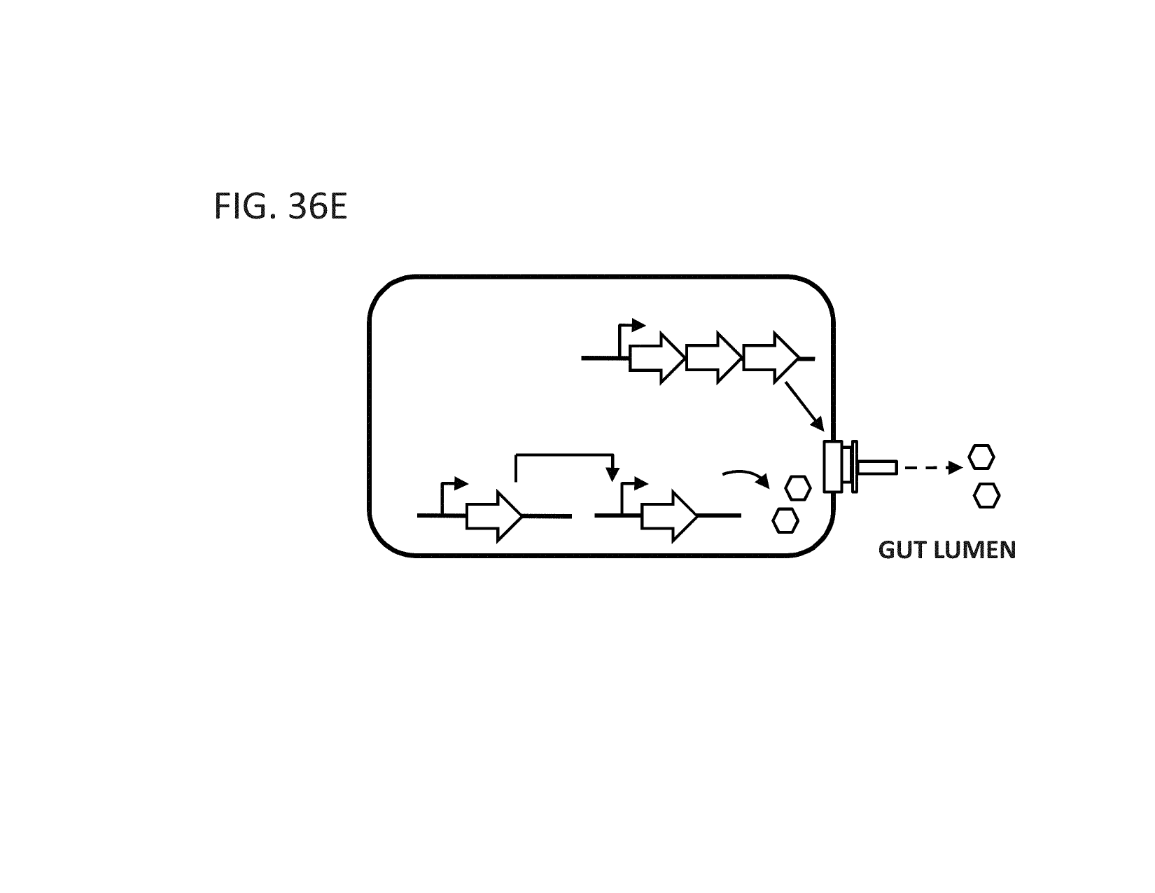

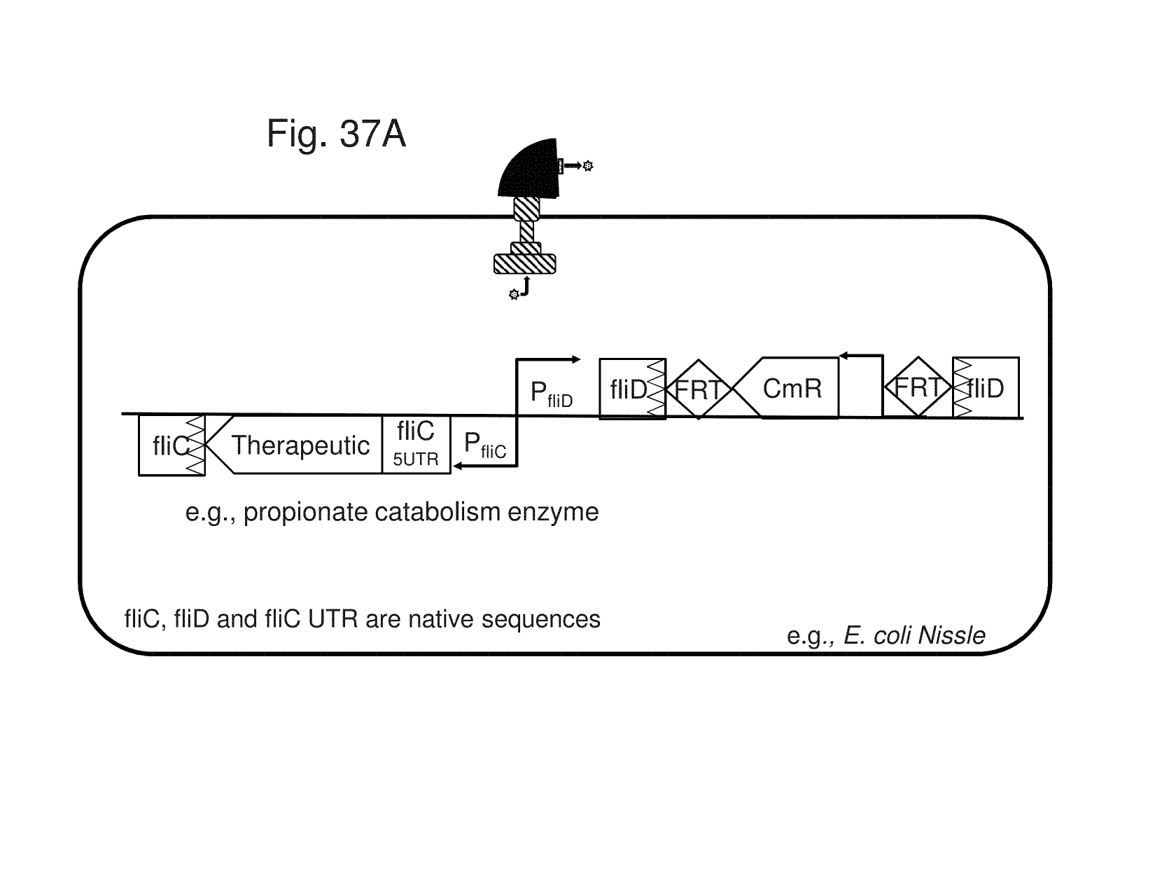

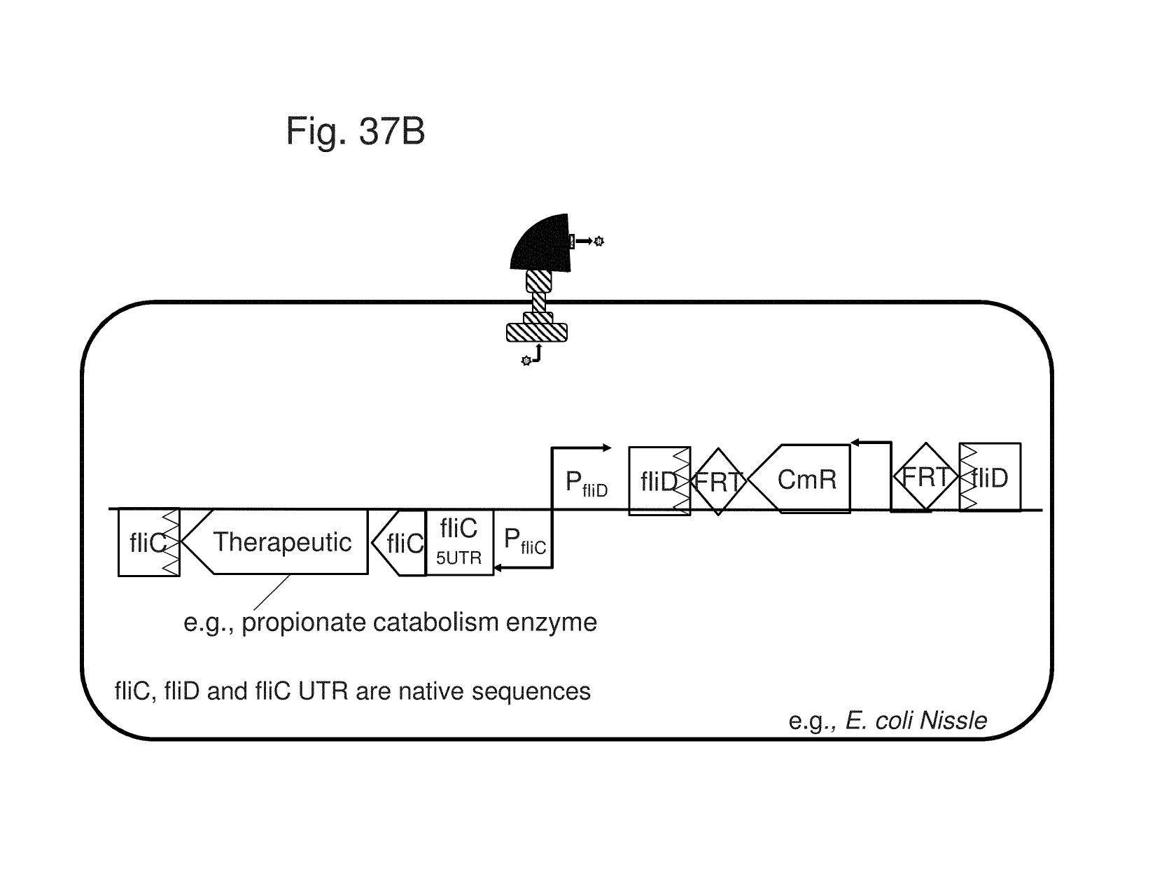

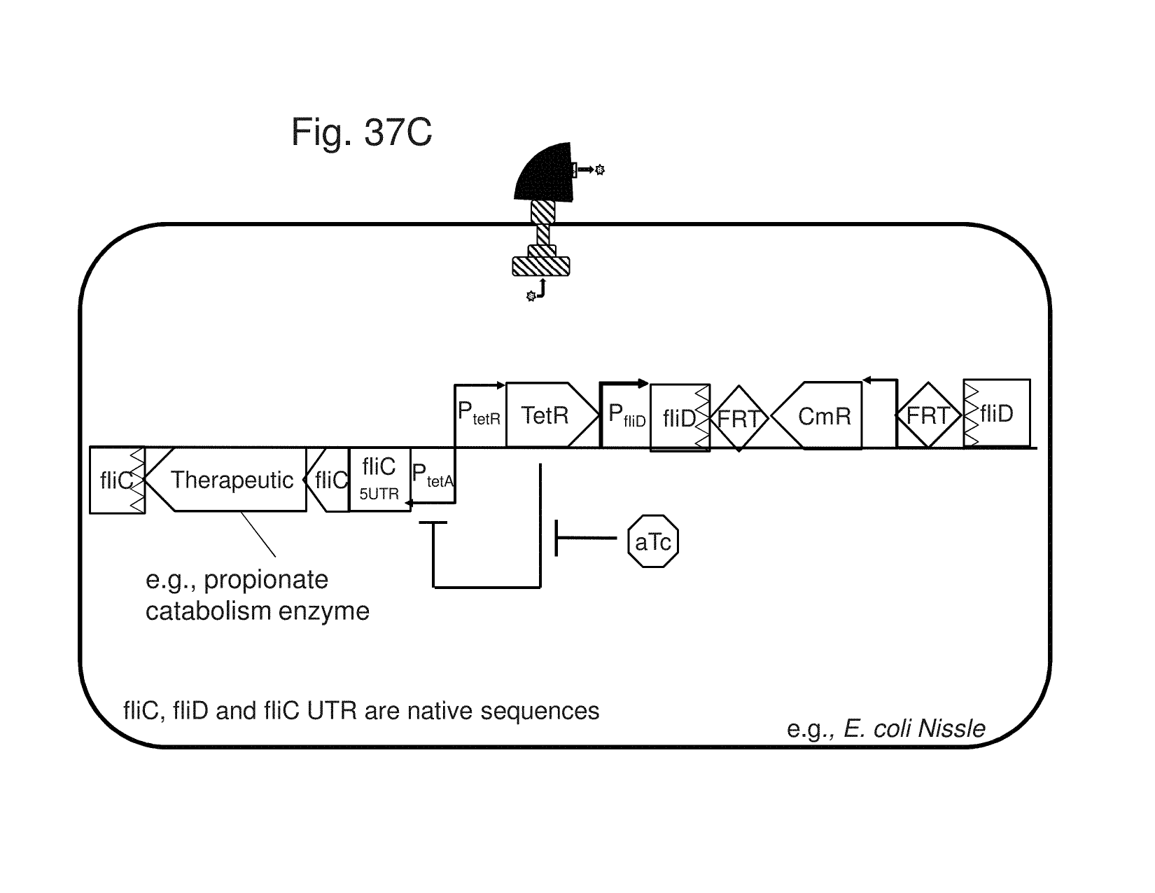

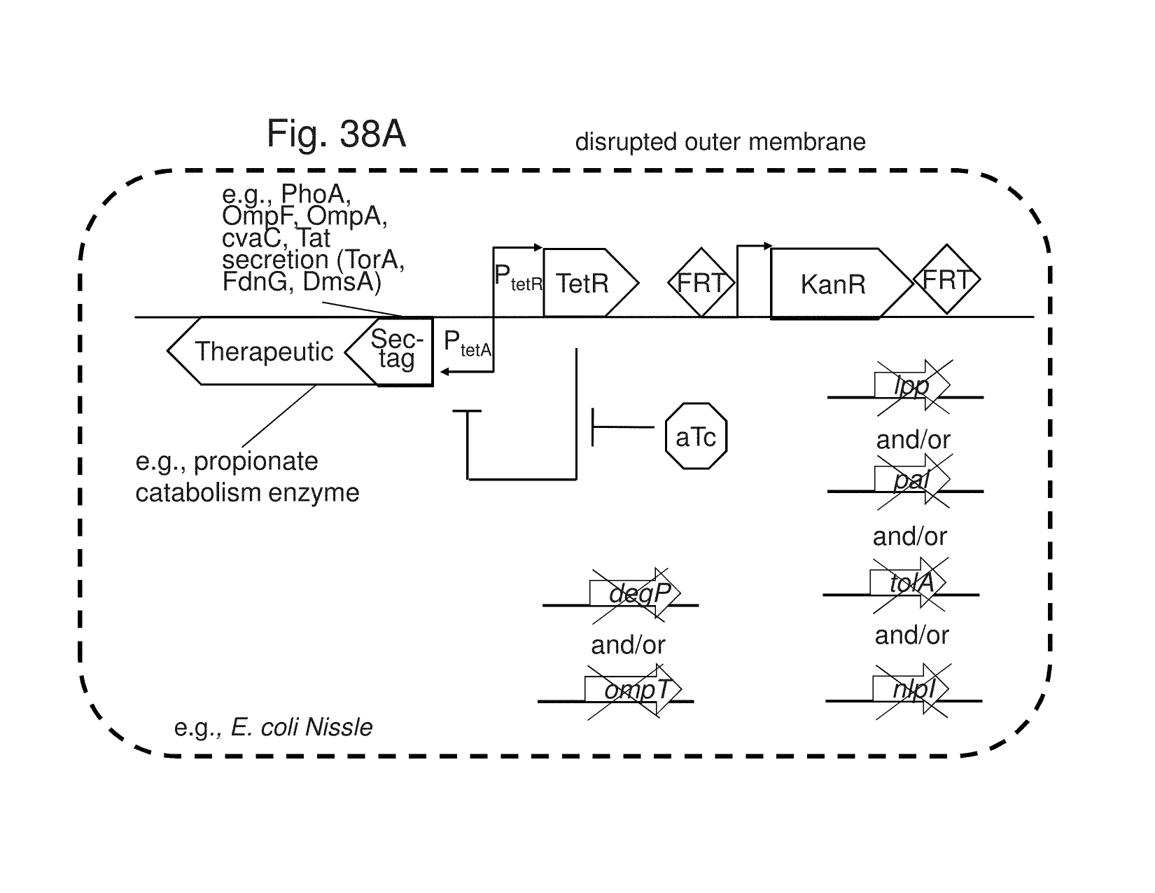

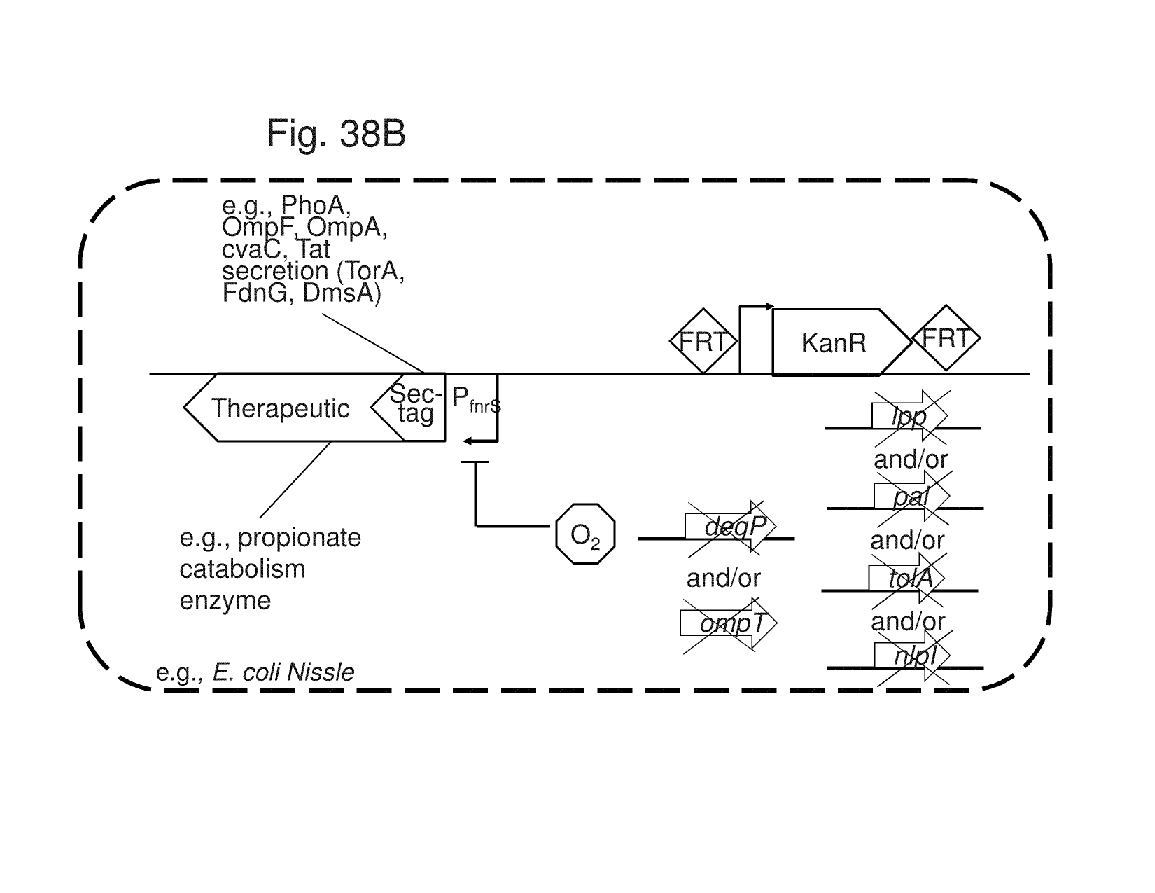

In Gram-negative bacteria, secretion machineries may span one or both of the inner and outer membranes. In some embodiments, the genetically engineered bacteria further comprise a non-native double membrane-spanning secretion system. Double membrane-spanning secretion systems include, but are not limited to, the type I secretion system (T1SS), the type II secretion system (T2SS), the type III secretion system (T3SS), the type IV secretion system (T4SS), the type VI secretion system (T6SS), and the resistance-nodulation-division (RND) family of multi-drug efflux pumps (Pugsley 1993; Gerlach et al., 2007; Collinson et al., 2015; Costa et al., 2015; Reeves et al., 2015; WO2014138324A1, incorporated herein by reference). Examples of such secretion systems are shown in FIG. 36A, FIG. 36B, FIG. 36C, FIG. 36D, and FIG. 36E, FIG. 37A, FIG. 37, FIG. 37C, FIG. FIG., and FIG. 38. Mycobacteria, which have a Gram-negative-like cell envelope, may also encode a type VII secretion system (T7SS) (Stanley et al., 2003). With the exception of the T2SS, double membrane-spanning secretions generally transport substrates from the bacterial cytoplasm directly into the extracellular space or into the target cell. In contrast, the T2SS and secretion systems that span only the outer membrane may use a two-step mechanism, wherein substrates are first translocated to the periplasm by inner membrane-spanning transporters, and then transferred to the outer membrane or secreted into the extracellular space. Outer membrane-spanning secretion systems include, but are not limited to, the type V secretion or autotransporter system or autosecreter system (TSSS), the curli secretion system, and the chaperone-usher pathway for pili assembly (Saier, 2006; Costa et al., 2015).

In some embodiments in which the one or more proteins of interest or therapeutic proteins are secreted or exported from the bacterium, the engineered bacterium comprises gene sequence(s) that includes a secretion tag. In some embodiments, the one or more proteins of interest or therapeutic proteins include a “secretion tag” of either RNA or peptide origin to direct the one or more proteins of interest or therapeutic proteins to specific secretion systems. For example, a secretion tag for the Type I Hemolysin secretion system is encoded in the C-terminal 53 amino acids of the alpha hemolysin protein (HlyA).

In some embodiments, a Hemolysin-based Secretion System is used to secrete the molecule of interest, e.g., therapeutic peptide. Type I Secretion systems offer the advantage of translocating their passenger peptide directly from the cytoplasm to the extracellular space, obviating the two-step process of other secretion types. FIG. 36C shows the alpha-hemolysin (HlyA) of uropathogenic Escherichia coli. This pathway uses HlyB, an ATP-binding cassette transporter; HlyD, a membrane fusion protein; and TolC, an outer membrane protein. The assembly of these three proteins forms a channel through both the inner and outer membranes. HlyB inserts into inner membrane to form a pore, HlyD aligns HlyB with TolC (outer membrane pore) thereby forming a channel through inner and outer membrane. Natively, this channel is used to secrete HlyA, however, to secrete the therapeutic peptide of the present disclosure, the secretion signal-containing C-terminal portion of HlyA is fused to the C-terminal portion of a therapeutic peptide (star) to mediate secretion of this peptide. The C-terminal secretion tag can be removed by either an autocatalytic or protease-catalyzed e.g., OmpT cleavage thereby releasing the one or more proteins of interest or therapeutic proteins into the extracellular milieu. In some embodiments, the one or more propionate catabolism enzyme(s) are expressed as a fusion protein with the 53 amino acids of the C termini of alpha-hemolysin (hlyA) of E. coli CFT073 (C terminal secretion tag).

In some embodiments, a Type V Autotransporter Secretion System is used to secrete the molecule of interest, e.g., therapeutic peptide. The Type V Auto-secretion System utilizes an N-terminal Sec-dependent peptide tag (inner membrane) and C-terminal tag (outer-membrane). This system uses the Sec-system to get from the cytoplasm to the periplasm. The C-terminal tag then inserts into the outer membrane forming a pore through which the “passenger protein” threads through. Due to the simplicity of the machinery and capacity to handle relatively large protein fluxes, the Type V secretion system is attractive for the extracellular production of recombinant proteins. As shown in FIG. 36B, a therapeutic peptide (star) can be fused to an N-terminal secretion signal, a linker, and the beta-domain of an autotransporter. The N-terminal, Sec-dependent signal sequence directs the protein to the SecA-YEG machinery which moves the protein across the inner membrane into the periplasm, followed by subsequent cleavage of the signal sequence. The Beta-domain is recruited to the Bam complex (‘Beta-barrel assembly machinery’) where the beta-domain is folded and inserted into the outer membrane as a beta-barrel structure. The therapeutic peptide is threaded through the hollow pore of the beta-barrel structure ahead of the linker sequence. Once across the outer membrane, the passenger is released from the membrane-embedded C-terminal tag by either an autocatalytic, intein-like mechanism (left side of Bam complex) or via a membrane-bound protease (black scissors; right side of Bam complex) (i.e., OmpT). For example, a membrane-associated peptidase to a complimentary protease cut site in the linker. Thus, in some embodiments, the secreted molecule, such as a propionate catabolism enzyme described herein, comprises an N-terminal secretion signal, a linker, and beta-domain of an autotransporter so as to allow the molecule to be secreted from the bacteria.

The N-terminal tag is removed by the Sec system. Thus, in some embodiments, the secretion system is able to remove this tag before secreting the one or more proteins of interest or therapeutic proteins, from the engineered bacteria. In the Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the anti-cancer molecule(s) into the extracellular milieu.

In some embodiments, the genetically engineered bacteria of the invention comprise a type III or a type III-like secretion system (T3SS) from Shigella, Salmonella, E. coli, Bivrio, Burkholderia, Yersinia, Chlamydia, or Pseudomonas. The traditional T3SS is capable of transporting a protein from the bacterial cytoplasm to the host cytoplasm through a needle complex. In the Type III traditional secretion system, the basal body closely resembles the flagella, however, instead of a “tail”/whip, the traditional T3SS has a syringe to inject the passenger proteins into host cells. The secretion tag is encoded by an N-terminal peptide (lengths vary and there are several different tags, see PCT/US14/020972). The N-terminal tag is not removed from the polypeptides in this secretion system.

The T3SS may be modified to secrete the molecule from the bacterial cytoplasm, but not inject the molecule into the host cytoplasm. Thus, the molecule is secreted into the gut lumen or other extracellular space. In some embodiments, the genetically engineered bacteria comprise said modified T3SS and are capable of secreting the propionate catabolism enzyme from the bacterial cytoplasm. In some embodiments, the secreted molecule, such as a propionate catabolism enzyme, comprises a type III secretion sequence that allows the propionate catabolism enzyme to be secreted from the bacteria.

In the Flagellar modified Type III Secretion, the tag is encoded in 5′ untranslated region of the mRNA and thus there is no peptide tag to cleave/remove. This modified system does not contain the “syringe” portion and instead uses the basal body of the flagella structure as the pore to translocate across both membranes and out through the forming flagella. If the fliC/fliD genes (encoding the flagella “tail”/whip) are disrupted the flagella cannot fully form and this promotes overall secretion. In some embodiments, the tail portion can be removed entirely.

In some embodiments, a flagellar type III secretion pathway is used to secrete the molecule of interest, e.g., a propionate catabolism enzyme. In some embodiments, an incomplete flagellum is used to secrete a therapeutic peptide of interest by recombinantly fusing the peptide to an N-terminal flagellar secretion signal of a native flagellar component. In this manner, the intracellularly expressed chimeric peptide can be mobilized across the inner and outer membranes into the surrounding host environment.

For example, a modified flagellar type III secretion apparatus in which untranslated DNA fragment upstream of the gene fliC (encoding flagellin), e.g., a 173-bp region, is fused to the gene encoding the heterologous protein or peptide can be used to secrete polypeptides of interest (See, e.g., Majander et al., Extracellular secretion of polypeptides using a modified Escherichia coli flagellar secretion apparatus. Nat Biotechnol. 2005 April; 23(4):475-81). In some cases, the untranslated region from the fliC loci may not be sufficient to mediate translocation of the passenger peptide through the flagella. Here it may be necessary to extend the N-terminal signal into the amino acid coding sequence of FliC, for example, by using the 173 bp of untranslated region along with the first 20 amino acids of FliC (see, e.g., Duan et al., Secretion of Insulinotropic Proteins by Commensal Bacteria: Rewiring the Gut To Treat Diabetes, Appl. Environ. Microbiol. December 2008 vol. 74 no. 23 7437-7438).

In alternate embodiments, the genetically engineered bacteria further comprise a non-native single membrane-spanning secretion system. Single membrane-spanning transporters may act as a component of a secretion system, or may export substrates independently. Such transporters include, but are not limited to, ATP-binding cassette translocases, flagellum/virulence-related translocases, conjugation-related translocases, the general secretory system (e.g., the SecYEG complex in E. coli), the accessory secretory system in mycobacteria and several types of Gram-positive bacteria (e.g., Bacillus anthracis, Lactobacillus johnsonii, Corynebacterium glutamicum, Streptococcus gordonii, Staphylococcus aureus), and the twin-arginine translocation (TAT) system (Saier, 2006; Rigel and Braunstein, 2008; Albiniak et al., 2013). It is known that the general secretory and TAT systems can both export substrates with cleavable N-terminal signal peptides into the periplasm, and have been explored in the context of biopharmaceutical production. The TAT system may offer particular advantages, however, in that it is able to transport folded substrates, thus eliminating the potential for premature or incorrect folding. In certain embodiments, the genetically engineered bacteria comprise a TAT or a TAT-like system and are capable of secreting the anti-cancer molecule of interest from the bacterial cytoplasm. One of ordinary skill in the art would appreciate that the secretion systems disclosed herein may be modified to act in different species, strains, and subtypes of bacteria, and/or adapted to deliver different payloads.

In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

One way to secrete properly folded proteins in gram-negative bacteria—particularly those requiring disulphide bonds—is to target the reducing-environment periplasm in conjunction with a destabilizing outer membrane. In this manner the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the genetically engineered bacteria have a “leaky” or de-stabilized outer membrane. Destabilizing the bacterial outer membrane to induce leakiness can be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp is the most abundant polypeptide in the bacterial cell existing at ˜500,000 copies per cell and functions as the primary ‘staple’ of the bacterial cell wall to the peptidoglycan. 1. Silhavy, T. J., Kahne, D. & Walker, S. The bacterial cell envelope. Cold Spring Harb Perspect Biol 2, a000414 (2010). TolA-PAL and OmpA complexes function similarly to Lpp and are other deletion targets to generate a leaky phenotype. Additionally, leaky phenotypes have been observed when periplasmic proteases are inactivated. The periplasm is very densely packed with protein and therefore encode several periplasmic proteins to facilitate protein turnover. Removal of periplasmic proteases such as degS, degP or nlpI can induce leaky phenotypes by promoting an excessive build-up of periplasmic protein. Mutation of the proteases can also preserve the effector polypeptide by preventing targeted degradation by these proteases. Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated lpp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes. in some embodiments, the engineered bacteria have one or more deleted or mutated periplasmic protease genes. In some embodiments, the engineered bacteria have one or more deleted or mutated periplasmic protease genes selected from degS, degP, and nlpl. In some embodiments, the engineered bacteria have one or more deleted or mutated gene(s), selected from lpp, ompA, ompF, tolA, tolB, pal, degS, degP, and nlpl genes.

To minimize disturbances to cell viability, the leaky phenotype can be made inducible by placing one or more membrane or periplasmic protease genes, e.g., selected from lpp, ompA, ompF, tolA, tolB, pal, degS, degP, and nlpl, under the control of an inducible promoter. For example, expression of lpp or other cell wall stability protein or periplasmic protease can be repressed in conditions where the therapeutic polypeptide needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., overexpression of colicins or the third topological domain of TolA, wherein peptide overexpression can be induced in conditions in which the therapeutic polypeptide needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

Table 21 and Table 22 below lists secretion systems for Gram positive bacteria and Gram negative bacteria.

TABLE 21
Secretion systems for gram positive bacteria

| Bacterial Strain | Relevant Secretion System |
|---|---|
| C. novyi-NT (Gram+) | Sec pathway |
| | Twin-arginine (TAT) pathway |
| C. butryicum (Gram+) | Sec pathway |
| | Twin-arginine (TAT) pathway |
| Listeria monocylogenes (Gram +) | Sec pathway |
| | Twin-arginine (TAT) pathway |

TABLE 22
Secretion Systems for Gram negative bacteria
Protein secretary pathways (SP) in gram-negative bacteria and their
descendants

| | | | | | | | |
|---|---|---|---|---|---|---|---|
| | | | | | | # | |
| Type | | | | | | Proteins/ | Energy |
| (Abbreviation) | Name | TC#² | Bacteria | Archaea | Eukarya | System | Source |

IMPS - Gram-negative bacterial inner membrane channel-forming translocases

| | | | | | | | |
|---|---|---|---|---|---|---|---|
| ABC | ATP binding | 3.A.1 | + | + | + | 3-4 | ATP |
| (SIP) | cassette | | | | | | |
| | translocase | | | | | | |
| SEC | General | 3.A.5 | + | + | + | ~12 | GTP |
| (IISP) | secretory | | | | | | OR |
| | translocase | | | | | | ATP + |
| | | | | | | | PMF |
| Fla/Path | Flagellum/virulence- | 3.A.6 | + | − | − | >10 | ATP |
| (IIISP) | related | | | | | | |
| | translocase | | | | | | |
| Conj | Conjugation- | 3.A.7 | + | − | − | >10 | ATP |
| (IVSP) | related | | | | | | |
| | translocase | | | | | | |
| Tat (IISP) | Twin- | 2.A.64 | + | + | +(chloroplasts) | 2-4 | PMF |
| | arginine | | | | | | |
| | targeting | | | | | | |
| | translocase | | | | | | |
| Oxa1 | Cytochrome | 2.A.9 | + | + | +(mitochondria | 1 | None |
| (YidC) | oxidase | | | | chloroplasts) | | or |
| | biogenesis | | | | | | PMF |
| | family | | | | | | |
| MscL | Large | 1.A.22 | + | + | + | 1 | None |
| | conductance | | | | | | |
| | mechanosensitive | | | | | | |
| | channel | | | | | | |
| | family | | | | | | |
| Holins | Holin | 1.E.1·21 | + | − | − | 1 | None |
| | functional | | | | | | |
| | superfamily | | | | | | |

Eukaryotic Organelles

| | | | | | | | |
|---|---|---|---|---|---|---|---|
| MPT | Mitochondrial | 3.A.B | − | − | +(mitochondrial) | >20 | ATP |
| | protein | | | | | | |
| | translocase | | | | | | |
| CEPT | Chloroplast | 3.A.9 | (+) | − | +(chloroplasts) | ≧3 | GTP |
| | envelope | | | | | | |
| | protein | | | | | | |
| | translocase | | | | | | |
| Bcl-2 | Eukaryotic | 1.A.21 | − | − | + | 1? | None |
| | Bcl-2 family | | | | | | |
| | (programmed | | | | | | |
| | cell death) | | | | | | |

Gram-negative bacterial outer membrane channel-forming translocases

| | | | | | | | |
|---|---|---|---|---|---|---|---|
| MTB | Main | 3.A.15 | +^{b} | − | − | ~14 | ATP; |
| (IISP) | terminal | | | | | | PMF |
| | branch of the | | | | | | |
| | general | | | | | | |
| | secretory | | | | | | |
| | translocase | | | | | | |
| FUP AT-1 | Fimbrial | 1.B.11 | +^{b} | − | − | 1 | None |
| | usher protein | 1.B.12 | +^{b} | | − | 1 | None |
| | Autotransporter-1 | | | | | | |
| AT-2 | Autotransporter-2 | 1.B.40 | +^{b} | − | − | 1 | None |
| OMF | | 1.B.17 | +^{b} | | +(?) | 1 | None |
| (ISP) | | | | | | | |
| TPS | | 1.B.20 | + | − | + | 1 | None |
| Secretin | | 1.B.22 | +^{b} | | − | 1 | None |
| (IISP and | | | | | | | |
| IISP) | | | | | | | |
| OmpIP | Outer | 1.B.33 | + | − | +(mitochondria; | ≧4 | None ? |
| | membrane | | | | chloroplasts) | | |
| | insertion | | | | | | |
| | porin | | | | | | |

The above tables for gram positive and gram negative bacteria list secretion systems that can be used to secrete polypeptides, e.g., propionate catabolism enzyme from the engineered bacteria, which are reviewed in Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety.

In some embodiments, one or more propionate catabolic enzymes described herein are secreted. In some embodiments, the genetically engineered bacterial comprise a native or non-native secretion system described herein for the secretion of one or more propionate catabolic enzymes described herein. Examplary Secretion Tags are shown in Table 23.

TABLE 23
Polypeptide Sequences of Exemplary Secretion Tags

| Description | Sequence |
|---|---|
| PhoA | MKQSTIALALLPLLFTPVTKA |
| SEQ ID NO: 299 | |
| PhoA | KQSTIALALLPLLFTPVTKA |
| SEQ ID NO: 300 | |
| OmpF | MMKRNILAVIVPALLVAGTANA |
| SEQ ID NO: 301 | |
| cvaC | MRTLTLNELDSVSGG |
| SEQ ID NO: 302 | |
| TorA | MNNNDLFQASRRRFLAQLGGLTVAGMLGTSLLTPRRATAAQAA |
| SEQ ID NO: 303 | |
| fdnG | MDVSRRQFFKICAGGMAGTTVAALGFAPKQALA |
| SEQ ID NO: 304 | |
| dmsA | MKTKIPDAVLAAEVSRRGLVKTTAIGGLAMASSALTLPFSRIAHA |
| SEQ ID NO: 305 | |
| PelB | KYLLPTAAAGLLLLAAQPAMA |
| SEQ ID NO: 306 | |
| HlyA secretion | LNPLINEISKIISAAGNFDVKEERAAASLLQLSGNASDFSYGRNSI |
| signal | TLTASA |
| SEQ ID NO: 307 | |
| HlyA secretion | CTTAATCCATTAATTAATGAAATCAGCAAAATCATTTCAGCT |
| signal | GCAGGTAATTTTGATGTTAAAGAGGAAAGAGCTGCAGCTTC |
| SEQ ID NO: 308 | TTTATTGCAGTTGTCCGGTAATGCCAGTGATTTTTCATATGG |
| | ACGGAACTCAATAACTTTGACAGCATCAGCATAA. |

In some embodiments, genetically engineered bacteria comprise a nucleic acid sequence that encodes a polypeptide which is at least about 80%, at least about 85%, at least about 90%, at least about 95%, or at least about 99% homologous to the DNA sequence of SEQ ID NO: 299, SEQ ID NO: 300, SEQ ID NO: 301, SEQ ID NO: 302 SEQ ID NO: 303, SEQ ID NO: 304, SEQ ID NO: 305, SEQ ID NO: 306, SEQ ID NO: 307, and/or SEQ ID NO: 308.

Any secretion tag or secretion system can be combined with any cytokine described herein, and can be used to generate a construct (plasmid based or integrated) which is driven by an directly or indirectly inducible or constitutive promoter described herein. In some embodiments, the secretion system is used in combination with one or more genomic mutations, which leads to the leaky or diffusible outer membrane phenotype (DOM), including but not limited to, lpp, nlP, tolA, PAL.

In some embodiments, the secretion system is selected from the type III flagellar, modified type III flagellar, type I (e.g., hemolysin system), type II, type IV, type V, type VI, and type VII secretion systems, resistance-nodulation-division (RND) multi-drug efflux pumps, a single membrane secretion system, Sec and, TAT secretion systems.

Any of the secretion systems described herein may according to the disclosure be employed to secrete the polypeptides of interest. In some embodiments,

In some embodiments, the genetically engineered bacteria are capable of expressing and secreting any one or more of the propionate catabolism enzymes and circuits described herein, in low-oxygen conditions, and/or in the presence of molecules or metabolites associated with PA and/or MMA, and/or in the presence of chemical and/or nutritional inducers that may or may not be present in the gut, and/or in the presence of metabolites that may or may not be present in vivo. In some embodiments, the bacteria are capable or expressing and secreting one or more propionate catabolism enzymes under conditions induced during in vitro strain culture, expansion, production and/or manufacture, such as the presence of arabinose and chemical and/or nutritional inducers described herein. In some embodiments, the gene sequences(s) are controlled by a promoter inducible by such in vivo or in vitro conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In some embodiments, any one or more of the described propionate catabolism secretion circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more transporters described herein (8) one or more exporters described herein, (9) combinations of one or more of such additional circuits.

These polypeptides may be mutated to increase stability, resistance to protease digestion, and/or activity.

TABLE 24
Comparison of Secretion systems for secretion of polypeptide
from engineered bacteria

| Secretion | | | | |
|---|---|---|---|---|
| System | Tag | Cleavage | Advantages | Other features |
| Modified | mRNA | No | No peptide tag | May not be as suited |
| Type III | (or N- | cleavage | Endogenous | for larger proteins |
| (flagellar) | terminal) | necessary | | Deletion of flagellar |
| | | | | genes |
| Type V | N- and | Yes | Large proteins | 2-step secretion |
| auto- | C- | | Endogenous | |
| transport | terminal | | Cleavable | |
| Type I | C- | No | | Tag; Exogenous |
| | terminal | | | Machinery |
| Diffusible | N- | Yes | Disulfide bond | May affect cell |
| Outer | terminal | | formation | fragility/ |
| Membrane | | | | survivability/ |
| (DOM) | | | | growth/yield |

In some embodiments, the therapeutic polypeptides of interest are secreted using components of the flagellar type III secretion system. In a non-limiting example, such a therapeutic polypeptide of interest is assembled behind a fliC-5′UTR (e.g., 173-bp untranslated region from the fliC loci), and is driven by the native promoter. In other embodiments, the expression of the therapeutic peptide of interested secreted using components of the flagellar type III secretion system is driven by a tet-inducible promoter. In alternate embodiments, an inducible promoter such as oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose is used. In some embodiments, the therapeutic polypeptide of interest is expressed from a plasmid (e.g., a medium copy plasmid). In some embodiments, the therapeutic polypeptide of interest is expressed from a construct which is integrated into fliC locus (thereby deleting fliC), where it is driven by the native FliC promoter. In some embodiments, an N terminal part of FliC (e.g., the first 20 amino acids of FliC) is included in the construct, to further increase secretion efficiency.

In some embodiments, the therapeutic polypeptides of interest, e.g., propionate catabolism enzymes, are secreted using via a diffusible outer membrane (DOM) system. In some embodiments, the therapeutic polypeptide of interest is fused to a N-terminal Sec-dependent secretion signal. Non-limiting examples of such N-terminal Sec-dependent secretion signals include PhoA, OmpF, OmpA, and cvaC. In alternate embodiments, the therapeutic polypeptide of interest is fused to a Tat-dependent secretion signal. Exemplary Tat-dependent tags include TorA, FdnG, and DmsA.

In certain embodiments, the genetically engineered bacteria comprise deletions or mutations in one or more of the outer membrane and/or periplasmic proteins. Non-limiting examples of such proteins, one or more of which may be deleted or mutated, include lpp, pal, tolA, and/or nlpI. In some embodiments, lpp is deleted or mutated. In some embodiments, pal is deleted or mutated. In some embodiments, tolA is deleted or mutated. In other embodiments, nlpl is deleted or mutated. In yet other embodiments, certain periplasmic proteases are deleted or mutated, e.g., to increase stability of the polypeptide in the periplasm. Non-limiting examples of such proteases include degP and ompT. In some embodiments, degP is deleted or mutated. In some embodiments, ompT is deleted or mutated. In some embodiments, degP and ompT are deleted or mutated.

In some embodiments, the therapeutic polypeptides of interest, e.g. are secreted via a Type V Auto-secreter (pie Protein) Secretion. In some embodiments, the therapeutic protein of interest is expressed as a fusion protein with the native Nissle auto-secreter E. coli_01635 (where the original passenger protein is replaced with the therapeutic polypeptides of interest.

In some embodiments, the therapeutic polypeptides of interest, e.g., propionate catabolism enzymes, are secreted via Type I Hemolysin Secretion. In one embodiment, therapeutic polypeptide of interest is expressed as fusion protein with the 53 amino acids of the C terminus of alpha-hemolysin (hlyA) of E. coli CFT073.

In some embodiments, one or more propionate catabolic enzymes described herein are secreted. In some embodiments, the one or more propionate catabolic enzymes described herein are further modified to improve secretion efficiency, decreased susceptibility to proteases, stability, and/or half-life. In some embodiments, PrpE is secreted, alone or in combination other propionate catabolic enzymes, one or more of accA1, pccB, mmcE, mutA, and mutB and/or one or more of prpB, prpC, prpD, and/or one or more of phaB, phaC, phaA. In some embodiments, one or more of accA1, pccB, mmcE, mutA, mutB are secreted. In some embodiments, one or more of prpB, prpC, prpD are secreted. In some embodiments, one or more of phaB, phaC, phaA are secreted.

Alternatively, any of the enzymes expressed by the genes described herein, e.g., in FIG. 9, FIG. 10, FIG. 15, and FIG. 20 may be combined.

Surface Display

In some embodiments, the genetically engineered bacteria and/or microorganisms encode one or more gene(s) and/or gene cassette(s) encoding a propionate catabolism enzyme which is anchored or displayed on the surface of the bacteria and/or microorganisms. In some embodiments, the one or more propionate catabolic enzymes described herein are further modified to improve display efficiency, decreased susceptibility to proteases, stability, and/or half-life. In some embodiments, PrpE is displayed on the cell surface, alone or in combination other propionate catabolic enzymes, e.g. With one or more of accA1, pccB, mmcE, mutA, and mutB and/or one or more of prpB, prpC, prpD, and/or one or more of phaB, phaC, phaA. In some embodiments, one or more of accA1, pccB, mmcE, mutA, mutB are displayed on the cell surface. In some embodiments, one or more of prpB, prpC, prpD are displayed on the cell surface. In some embodiments, one or more of phaB, phaC, phaA are displayed on the cell surface.

In some embodiments, the genetically engineered bacteria comprise one or more gene sequence(s) encoding a propionate catabolism enzyme, which is anchored or displayed on the surface of the bacteria, and which remains anchored while exerting its effector function. In other embodiments, the genetically engineered bacteria encoding the surface-displayed therapeutic polypeptide, e.g., propionate catabolism enzyme(s), lyse before, during or after exerting their effector function. In some embodiments, the genetically engineered bacteria encode a propionate catabolism enzyme that is temporarily attached to the cell surface and which dissociates from the bacterium before, during, or after exerting its function.

In some embodiments, shorter peptides or polypeptides, e.g. peptides or polypeptides of less than 60 amino acids of length, are displayed on the cell surface of the genetically engineered bacteria. In some embodiments, such shorter peptides or polypeptides comprise a propionate catabolism enzyme.

Several strategies for the display of shorter peptides or polypeptides on the surface of gram negative bacteria are known in the art, and are for example described in Georgiou et al., Display of heterologous proteins on the surface of microorganisms: from the screening of combinatorial libraries to live recombinant vaccines: Nat Biotechnol. 1997 January; 15(1):29-34, the contents of which is herein incorporated by reference in its entirety. These systems all share a common theme, targeting recombinant proteins to the cell surface by the construction of gene fusions using sequences from membrane-anchoring domains of surface proteins. Non-limiting examples of such strategies are described in Table 25.

TABLE 25
Exemplary Cell Surface Display Strategies

| Carrier protein | Exemplary | Type of fusion | Localization of |
|---|---|---|---|
| LamB | E. coli | Sandwich fusion | Cell surface |
| PhoE | E. coli | Sandwich fusion | Cell surface |
| OprF | Pseudomonas | Sandwich fusion | Cell surface |
| Gram negative | E. coli | C-terminal or | Periplasmic side |
| [symbol] | | [symbol] | or outer |
| | | | [symbol] |
| Lpp-OmpA | E. coli | C-terminal fusion | Cell surface |
| VirG | Shigella | N-terminal fusion | Cell surface |
| IgA | Neisseria | N-terminal fusion | Cell surface |
| Flagellin (FliC) | E. coli | Sandwich fusion | Cell surface |
| Flagellin (FliC) | E. coli | Sandwich fusion | Cell surface |
| FimH (type I pili) | E. coli | Sandwich fusion | Cell surface |
| PapA (Pap pili) | E. coli | Sandwich fusion | Cell surface |
| PulA | Klebsiella | C-terminal fusion | Cell surface/ |
| | | | extracellular fluid |

[symbol]

indicates data missing or illegible when filed

In some embodiments, the genetically engineered bacteria comprise one or more gene sequence(s) encoding one or more short therapeutic peptides or polypeptides fused into surface exposed loops of outer membrane proteins (OMPs), e.g., from enteric bacteria. In a non-limiting example, the short therapeutic peptides or polypeptides expressed by the genetically engineered bacteria are inserted into the outer membrane protein LamB, e.g., from E. coli, and displayed on the bacterial cell surface. Extracellular display of peptides through Insertion of peptides into surface exposed loops of LamB is for example described in Hofnung et al., Expression of foreign polypeptides at the Escherichia coli cell surface; Methods Cell Biol. 34:77-105, and Charbit, A. et al., 1987. Presentation of two epitopes of the preS2 region of hepatitis B virus on live recombinant bacteria, J. Immunol. 139:1658-1664.

In another non-limiting example, the short therapeutic peptides or polypeptides encoded by one or more gene sequence(s) comprised in the genetically engineered bacteria are inserted into the outer membrane protein PhoE, e.g., from E. coli, and displayed on the bacterial cell surface. The PhoE protein is another abundant outer membrane protein of E. coli K-12, which has a trimeric structure and functions as a pore for small molecules. Analysis of the primary structure of PhoE revealed 16 beta sheets which traverse through the membranes, and eight hypervariable regions exposed at the surface of the cell. One or more of these cell surface exposed regions of PhoE protein can be used to insert heterologous peptides. For example, antigenic determinants of pathogenic organisms have been presented in one or more cell surface exposed regions of PhoE protein (e.g., as described in Aterberg et al., 1990; Outer membrane PhoE protein of Escherichia coli as a carrier for foreign antigenic determinants: immunogenicity of epitopes of foot-and-mouth disease virus; Vaccine. 1990 February; 8(1):85-91).

In some embodiments, the genetically engineered bacteria comprise one or more gene sequence(s) encoding one or more short therapeutic peptides or polypeptides fused to protein components of extracellular appendages. Several systems have been described, in which extracellular appendages, such as pili and flagella are used to display peptides of interest at the bacterial cell surface. Examples of flagellar and pilar proteins used include FliC, a major structural component of the E. coli flagellum, and PapA, the major subunit of the Pap pilus. In one embodiment, the genetically engineered bacteria comprise one or more gene sequence(s) encoding one or more components of a FLITRX system. The FLITRX system is an E. coli display system based on the use of fusion protein of FliC and thioredoxin, a small redox protein which represents a highly versatile scaffold that allows peptide inserts to assume a confirmation compatible with binding to other proteins. In the FLITRX system, thioredoxin is fused into a dispensable region of FliC. Then, heterologous peptides can be inserted within the thioredoxin domain in the FliC fusion, and are surface exposed. Other scaffolding proteins are known in the art, some of which may replace thioredoxin as a scaffolding protein in this system.

In some embodiments, the genetically engineered bacteria comprise a FimH fusion protein, in which the therapeutic peptide of interest is fused to FimH, an adhesin of type 1 fimbriae, e.g., from E. coli. FimH adhesin chimeras containing as many as 56 foreign amino acids in certain positions are transported to the bacterial surface as components of the fimbrial organelles (Pallesen et al., Chimeric FimH adhesion of type I fimbriae: a bacterial surface display system for heterologous sequences. Microbiology 141: 2839-2848).

In some embodiments, the genetically engineered bacteria comprise one or more gene sequence(s) encoding a fusion protein in which the therapeutic peptide of interest is fused to the major subunit of F11 fimbriae, e.g., from E. coli. Hypervariable regions of the major subunit of F11 fimbriae can be used for insertion of heterologous peptides, e.g., antigenic epitopes (Van Die et al., Expression of foreign epitopes in P-fimbriae of Escherichia coli. Mol. Gen. Genet. 222: 297-303).

In one embodiment, the genetically engineered bacteria comprise one or more gene sequence(s) encoding a papA fusion protein, in which the therapeutic peptide of interest is fused to papA. In some embodiments, peptides of interest are inserted following either codon 7 or 68 of the coding sequence for the mature portion of PapA, as peptides in the area of amino acids 7 and 68 of PapA are localized at the external side of the pilus (Steidler et al., Pap pili as a vector system for surface exposition of an immunoglobulin G-binding domain of protein A of Staphylococcus aureus in Escherichia coli; J Bacteriol. 1993 December; 175(23):7639-43).

In some embodiments, the genetically engineered bacteria comprise one or more gene sequence(s), which encode polypeptides larger than 60 amino acids, e.g., propionate catabolism enzyme(s), and which are displayed on the bacterial cell surface. In some embodiments, the genetically engineered bacteria comprise one or more gene sequence(s), which encode a fusion protein, in which a therapeutic peptide of interest, e.g., a polypeptide greater than 60 amino acids in length, is fused to a lipoprotein from a gram negative bacterium, or one or more fragments thereof.

In one embodiment, the genetically engineered bacteria comprise one or more gene sequence(s), which encode a fusion protein, in which a therapeutic protein of interest is fused to peptidoglycan associated lipoprotein (PAL) or a fragment thereof. The fusion protein in located in the periplasm and can be displayed externally upon permeabilization of the outer membrane. For example, a PAL-scFv fusion protein was shown to bind its antigen and to be tightly bound to the murein layer of the cell envelope (Fuchs et al., Targeting recombinant antibodies to the surface of Escherichia coli fusion to a peptidoglycan-associated lipoprotein; Biotechnology (N Y). 1991 December; 9(12):1369-72). The PAL-scFv fusion was located in the periplasm and bound to the murein layer, and after permeabilization of the outer membrane, the scFv became accessible to externally added antigen. In some embodiments, the genetically engineered bacteria comprising a fusion protein for surface display further have a permeable outer membrane. Mutations and/or deletions resulting in a leaky outer membrane are described elsewhere herein.

In one embodiment, the genetically engineered bacteria encode a fusion protein, in which a therapeutic protein of interest, e.g., immune modulatory effector, is fused to residues of the major lipoprotein of a gram-negative bacterium, e.g., E. coli. In one embodiment, the genetically engineered bacteria encode a fusion protein, in which a therapeutic protein of interest, is fused to the signal peptide and the nine N-terminal amino acid residues of the major lipoprotein of a gram-negative bacterium, e.g., E. coli. These residues of the E. coli major lipoprotein function as a hydrophobic membrane anchor. For example, a fusion construct of these residues with a therapeutic polypeptide, in this case a scFv fragment, resulted in specific accumulation of an immunoreactive and cell-bound polypeptide in E. coli (Laukkanen et al., Lipid-tagged antibodies: bacterial expression and characterization of a lipoprotein-single-chain antibody fusion protein. Mol. Microbiol. 4:1259-1268).

In one embodiment, the genetically engineered bacteria encode a fusion protein, in which a therapeutic protein of interest, is inserted into the TraT protein of a gram-negative bacterium, e.g., E. coli, e.g. at position 180. The TraT protein is a surface-exposed lipoprotein, specified by plasmids of the IncF group, that mediates serum resistance and surface exclusion. Taylor et al. showed that insertion of the C3 epitope of polio virus, e.g., at position 180, allowed exposure of the antigen to the cell surface, while the oligomeric conformation of the wild-type protein was maintained (Taylor et al., The TraT lipoprotein as a vehicle for the transport of foreign antigenic determinants to the cell surface of Escherichia coli K12: structure-function relationship in the TraT protein. Mol. Microbiol. 1990 August; 4(8):1259-68).

In one embodiment, the genetically engineered bacteria comprise one or more genes and/or gene cassettes encoding a fusion protein comprising a Lpp-OmpA display vehicle comprising the N terminal outer membrane signal from the major lipoprotein (Lpp) fused to a domain from the outer membrane protein OmpA, fused to the therapeutic polypeptide of interest. In this system, the Lpp signal peptide mediates localization, and OmpA provides the framework for the display of the therapeutic protein of interest. Lpp-OmpA fusions have been used to display several proteins between 20 and 54 kDa in size on the surface of E. coli (see, e.g., Staphopoulos et al., Characterization of Escherichia coli expressing and Lpp OmpA. (46-159)-PhoA fusion protein localized in the outer membrane). For example, Fransisco et al fused beta-lactamase to the N-terminal targeting sequence of Lpp and an OmpA fragment containing 5 of the 8 membrane spanning loops of the native protein. This fusion protein was assembled on the cell surface and the beta-lactamase domain was stably anchored in the cell wall (Fransisco et al., Transport ansd anchoring of beta-lactamase to the external surface of Escherichia coli; Proc. Natl. Acad. Sci. USA Vol 89, pp. 2713-2717, 1992).

In one embodiment, the Type II secretion pathway or a variation thereof is used to for transient or longer duration display of therapeutic proteins of interest on the bacterial cell surface, e.g., the IgA protease secretion pathway of Neisseria or the VirG protein pathway of Shigella. In one embodiment, the IgA protease secretion pathway is used to export and display therapeutic peptides of interest on the cell surface of gram negative bacteria. The IgA proteases of Neisseria gonorrhoeae and Hemophilus influenza use a variation of the most common, Type II secretion pathway, to achieve extracellular export independent of any other gene products. The IgA genes of Neisseria species encode extracellular proteins that cleave human IgA1 antibody. The iga gene alone is sufficient to direct selected extracellular secretion of IgA protease in Neisseria, Salmonella, and E. coli species (Klauser et al., 1993, Extracellular transport of cholera toxin B subunit using Neisseria IgA protease beta-domain—conformation-dependent outer membrane translocation. EMBO J 9:1991-1999, and references therein). The mature IgA protease is processed in several steps from a large precursor by signal peptidase and autoproteolytic cleavage. The precursor consists of four domains: (1) an aminoterminal signal peptide which mediates inner membrane transport; (2) the protease domain (3) the alpha domain, a basic alpha helical region which is secreted with the protease and (4) the autotransporter beta domain which harbors the essential function for outer membrane transport. Essentially, the C-terminal beta autotransporter domain of the IgA protease forms a channel in the outer membrane that mediates the export of the N terminal domain across the membrane, which in turn becomes transiently displayed on the external surface of the bacteria. The alpha domain and protease domain are then released through proteolytic cleavage. Klauser et al. (1993), showed that replacement of the native N-terminal domains of IgA protease of N. gonorrhoeae with the cholera toxin B resulted in the surface presentation of the passenger polypeptide in S. typhymurium. In another study, the signal sequence and the C-terminal beta autotransporter domain of the IgA protease of Neisseria gonorrhoeae was used to translocate and display a scFv directed against a porcine epidemic diarrhea virus epitope on the bacterial cell surface of E. coli (Pyo et al., Escherichia coli expressing single chain Fv on the cell surface as a potential prophylactic of porcine epidemic diarrhea virus; Vaccine (27) (2009) 2030-2036.).

Thus, in one embodiment, the genetically engineered bacteria encode a IgA protease fragment in which the alpha domain is substituted with a therapeutic protein of interest, and fused to a functional IgA protease beta-domain, which mediates export through the outer membrane. Without wishing to be bound by theory, IgA protease activity is eliminated in such a fusion protein, and therefore the autoproteolytic release of the fusion protein into the medium does not occur, resulting in the display of the therapeutic protein of interest on the cell surface of the gram-negative host bacterium.

The secretion of VirG protein from Shigella is similar to the export system utilized by the IgA protease of Neisseria (see, e.g., Suzuki et al., 1995; Extracellular transport of VirG protein in Shigella J Biol. Chem 270:30874-30880, and references therein). Thus, in some embodiments, the genetically engineered bacteria encode a fusion protein comprising a therapeutic protein of interest fused to the membrane spanning region of VirG, resulting in surface display of the therapeutic protein of interest. The VirG gene on the large plasmid of Shigella has been shown to be responsible for the localized deposition of filamentous actin (F-actin) trailing from one pole of invading bacterial cells and extending in a filament through the host epithelial cytoplasm. VirG is a surface-exposed outer membrane protein consisting of three distinctive domains, the N-terminal signal sequence (amino acids 1-52), the id α-domain (amino acids 53-758), and the dC-terminal β-core (amino acids 759-1102) (see, e.g., Suzuki et al., 1996; Functional Analysis of Shigella VirG Domains Essential for Interaction with Vinculin and Actin-based Motility; J. Biol. Chem., 271, 21878-21885, and references therein). Suzuki et al. (1995); showed that the fusion of a foreign protein such as MalE or PhoA protein to the N terminus 37-kDa VirG portion resulted in the transport of the passenger polypeptides from the periplasm to the external side of the outer membrane, indicating that the C-terminal 37-kDa VirG portion embedded in the outer membrane is involved in the translocation of the preceding VirG portion or the heterologous or passenger polypeptide from the periplasmic space to the external side of the outer membrane, in a manner homologous to the IgA protease beta-domain. In some embodiments, the genetically engineered bacteria comprise one or more gene(s) or gene cassette(s) encoding a fusion protein, in which a C-terminal 37-kDa VirG protein fragment is fused to a therapeutic protein of interest.

In some embodiments, the genetically engineered bacteria comprise one or more gene(s) or gene cassette(s) encoding a fusion protein, in which a therapeutic protein of interest is fused to pullulanase for temporary surface display. Pullulanase is specifically released into the medium by Klebsiella pneumoniae, and exists as a fully exposed, cell surface-bound intermediate before it is released into the medium from early stationary growth phase onwards. Cell-surface anchoring is accomplished by an N-terminal fatty acyl modification whose chemical composition is identical to that of other bacterial protein.

Unlike the IgA protease, the lipoprotein pullulanase (PulA) of Klebsiella pneumoniae, which is also exported via a type II secretion mechanism, requires 14 genes for its translocation across the outer membrane. For example, Pugsley and coworkers have shown that the lipoprotein pullulanase (PulA) can facilitate translocation of the periplasmic enzyme beta-lactamase across the outer membrane. In particular, in E. coli strains expressing all pullulanase secretion genes, pullulanase-beta-lactamase hybrid protein molecules containing an N-terminal 834-amino-acid pullulanase segment were efficiently transported to the cell surface. Of note, pullulanase hybrids remain only temporarily attached to the bacterial surface and are subsequently released into the medium (Kornacker and Pugsley: The normally periplasmic enzyme beta-lactamase is specifically and efficiently translocated through the Escherichia coli outer membrane when it is fused to the cell surface enzyme pullulanase. Mol. Microbiol. 4:1101-1109, and references therein). Accordingly, in some embodiments, the genetically engineered bacteria comprise one or more gene sequence(s) comprising a complete set of pullulanase genes required for secretion and fusion protein comprising a therapeutic protein of interest fused to a N-terminal pullulanase polypeptide fragment, e.g., as described by Kornacker and Pugsley. In some embodiments, the fusion proteins comprising N-terminal pullulanase polypeptide fused to the therapeutic protein of interest, are transiently displayed on the surface of the bacterial cell, and subsequently released into the media or extracellular space.

In one embodiment, the genetically engineered bacteria comprise one or more gene(s) or gene cassette(s) encoding a fusion protein in which the ice nucleation protein (INP) from Pseudomonas syringae anchors a therapeutic protein of interest in the cell wall. INP is a secretory protein that catalyzes extracellular ice formation as the ice nuclei. INP has been found in a number of Gram-negative species, including P. syringae, Erwinia herbicola, Xanthomonas campestris, and Pseudomonas fluorescens. Four genes in P. syringae strains, inaK, inaV, and inaZ, and inaQ exhibit high similarities in sequences and in primary organization (Li et al., Molecular Characterization of an Ice Nucleation Protein Variant (InaQ) from Pseudomonas syringae and the Analysis of Its Transmembrane Transport Activity in Escherichia coli Int J Biol Sci. 2012; 8(8): 1097-1108). All INPs (1200 aa to 1500 aa) comprise of three distinct structural domains: (1) the N-terminal domain (approximately 15% of the total sequence), which is relatively hydrophobic and which is are potentially capable of being coupled to the mannan-phosphatidylinositol group in the outer membrane through N-glycan (Asp) or O-glycan (Ser, Thr) linkages; (2) the C-terminal domain (approximately 4%), which is a relatively hydrophilic terminus; and (3) the central repeating domain (CRD) (approximately 81%), which constitutes contiguous repeats given by 16-residue (or 48-residue) periodicities with a consensus octapeptide (Ala-Gly-Tyr-Gly-Ser-Thr-Leu-Thr) (SEQ ID NO: 315). INPs have been employed in various bacterial cell-surface display systems including E. coli, Zymomonas mobilis, Salmonellas sp., Vibrio anguillarum, Pseudomonas putida, and cyanobacteria, in all of which INPs were able to target a heterologous protein onto the surface of the host cell. Moreover, the N-terminal region alone was shown to direct translocation of foreign proteins to the cell surface and can be employed as a potential cell surface display motif (Li et al., 2004 Functional display of foreign protein on surface of Escherichia coli using N-terminal domain of ice nucleation protein; Biotechnol Bioeng. 2004 Jan. 20; 85(2):214-21). Accordingly, in some embodiments, the genetically engineered bacteria comprise IMP fusions for surface display of a therapeutic peptide of interest. In some embodiments the N-terminal region of the INP protein is fused to the polypeptide of interest for surface display.

IMP proteins further have modifiable internal repeating units, i.e., CRD length is adjustable, which is allows flexibility in protein fusion length (Jung et al., 1998), and also can accommodate larger polypeptides. For example, the INP-based display systems were used to successfully express a 90 kDA protein on the cell surface of E. coli (Wu et al., 2006; Cell surface display of Chi92 on Escherichia coli using ice nucleation protein for improved catalytic and antifungal activity; FEMS Microbiology Letters, Volume 256, Issue 1; Pages 119-125).

It is understood by those skilled in the art that translocation of such fusion or hybrid proteins described herein requires a “translocation-competent” conformation, e.g., the formation of disulfide bonds, e.g., in the periplasmic space, may be undesirable and inhibit translocation through the outer membrane (see, e.g., Klauser et al., 1990), or alternatively may be required for, (or at least not impede) translocation through the outer membrane (see, e.g., Pugsley, 1992; Translocation of a folded protein across the outer membrane in Escherichia coli; Proc Natl Acad Sci USA. 1992 Dec. 15; 89(24): 12058-12062). In some embodiments, the genetically engineered bacteria comprise one or more gene sequence(s) encoding for a fusion protein in which disulfide bonds are prevented from forming prior to the translocation to the cell surface. In some embodiments, the genetically engineered bacteria comprise one or more gene(s) or gene cassette(s) encoding for a fusion protein in which disulfide bonds are formed prior to translocation to the cell surface.

Expression systems for the display of proteins in Gram-positive bacteria have also been developed. Consequently, in some embodiments, gram positive bacteria are engineered to display therapeutic proteins of interest on their cell surface. Uhlen et al. used fusions to the cell-wall bound, X-domain of protein A, for the display of foreign peptides up to 88 amino acids long to the surface of Staphylococcus strains. For example, one study describes an expression system to allow targeting of heterologous proteins to the cell surface of Staphylococcus xylosus, a coagulase-negative gram-positive bacterium (Hansson et al., Expression of recombinant proteins on the surface of the coagulase-negative bacterium Staphylococcus xylosus; J Bacteriol. 1992 July; 174(13):4239-45).

The expression of recombinant gene fragments, fused between gene fragments encoding the signal peptide and the cell surface-binding regions of staphylococcal protein A, targets the resulting fusion proteins to the outer bacterial cell surface via the membrane-anchoring region and the highly charged cell wall-spanning region of staphylococcal protein A. Accordingly, in some embodiments, the genetically engineered bacteria comprise one or more gene sequences encoding a therapeutic polypeptide fused between gene fragments encoding the signal peptide and the cell surface-binding regions of staphylococcal protein A

E. coli-staphylococcus shuttle vectors have been constructed by taking advantage of the promoter, signal sequence, and propeptide region from the lipase gene construct derived from S. hyicus and the cell surface attachment part of staphylococcal protein A. This system has been investigated for the surface display of heterologous polypeptides on S. carnosus (Samuelson et al., Cell surface display of recombinant proteins on Staphylococcus carnosus; J Bacteriol. 1995 March; 177(6):1470-6). In some embodiments, the genetically engineered bacteria comprise one or more gene sequence(s) encoding a therapeutic polypeptide fusion protein comprising promoter, signal sequence, and propeptide region from the lipase gene construct derived from S. hyicus and the cell surface attachment part of staphylococcal protein A.

In other studies, the fibrillary M6 proteins of Streptococcus pyrogenes was employed as a carrier for antigen delivery in Streptococcus cells. (Pozzi et al., 1992; Delivery and expression of a heterologous antigen on the surface of streptococci. Infect. Immunm. 60: 1902-1907). In some embodiments, the genetically engineered bacteria comprise one or more gene sequence(s) comprising therapeutic polypeptide fusion proteins comprising the fibrillary M6 proteins of Streptococcus pyrogenes for cell surface display of the therapeutic polypeptide.

In some embodiments, the genetically engineered bacteria comprise one or more gene(s) or gene cassette(s) encoding a polypeptide of interest which is displayed on the cell surface through a fusion with an intimin or invasin. Intimins and invasins belong to a family of bacterial adhesins which specifically interact with various eukaryotic cell surface receptors, thereby mediating bacterial adherence and invasion. Both intimins and invasins provide a structural scaffold ideally suited to the cell surface display.

In some embodiments, the genetically engineered bacteria comprise one or more gene(s) or gene cassette(s) encoding a polypeptide of interest which is displayed on the cell surface through a fusion with an intimin, e.g., with the Enterohemorrhagic E. coli Intimin EaeA protein or a carboxy-terminal truncation thereof (e.g., as described inWentzel et al, Display of Passenger Proteins on the Surface of Escherichia coli K-12 by the Enterohemorrhagic E. coli Intimin EaeA J Bacteriol. 2001 December; 183(24): 7273-7284). For example, N-terminal 489 amino acids of invasin are sufficient to promote the localization of a fusion protein to the cell surface. [030] In some embodiments, the genetically engineered bacteria comprise one or more gene(s) or gene cassette(s) encoding a polypeptide of interest which is displayed on the cell surface through a fusion with an invasin, e.g. Enterohemorrhagic E. coli invasion, or a carboxyterminal truncation thereof. For example, N-terminal 539 amino acids of intimin were sufficient to promote outer membrane localization of a fusion protein (Liu et al., The Tir-binding region of enterohaemorrhagic Escherichia coli intimin is sufficient to trigger actin condensation after bacterial-induced host cell signaling; Mol Microbiol. 1999 October; 34(1):67-81).

In some embodiments, the genetically engineered bacteria comprise one or more gene(s) or gene cassette(s) encoding a polypeptide of interest which is displayed on the cell surface through a fusion with Bacillus anthracis exosporal protein (BclA) as an anchoring motif. The BclA is an exosporium protein, a hair-like protein surrounding the B. anthracis spore. In a nonlimiting example, a polypeptide of interest is linked to the C-terminus of N-terminal domain (21 amino acids) of BclA, e.g., as described in Park et al. (Surface display of recombinant proteins on Escherichia coli by BclA exosporium of Bacillus anthracis).

Various other anchoring motifs have been developed including OprF, OmpC, and OmpX. In some embodiments, the genetically engineered bacteria comprise one or more gene(s) or gene cassette(s) encoding a polypeptide of interest which is displayed on the cell surface through a fusion with OprF, OmpC, and OmpX.

In some embodiments, the therapeutic polypeptides of interest are permanently displayed on the cell surface of the genetically engineered bacterium. In some embodiments, the therapeutic polypeptides of interest are transiently displayed on the cell surface of the genetically engineered bacterium.

In some embodiments, the therapeutic polypeptides are displayed in strains, e.g., described herein which display a leaky phenotype. Such strains have deactivating mutations in one or more of genes encoding a protein that tethers the outer membrane to the peptidoglycan skeleton, e.g., lpp, ompC, ompA, ompF, tolA, tolB, pal, and/or one or more genes encoding a periplasmic protease, e.g., degS, degP, nlpl.

In some embodiments, one or more a propionate catabolism enzyme(s) are displayed on the bacterial cell surface, alone or in combination with other therapeutic polypeptides of interest.

In some embodiments, a cell surface display strategy or circuit is combined with a secretion strategy or circuit in one bacterium. In some embodiments, the same polypeptide is both displayed and secreted. In some embodiments, a first polypeptide is displayed and a second is secreted. In some embodiments, a display strategy or circuit strategy is combined with a circuit for the intracellular production of an enzyme and consequentially intracellular catabolism of its substrate. In some embodiments, a display strategy or display circuit is combined with a circuit for the intracellular production of propionate catabolism enzyme.

In some embodiments, the expression of the surface displayed polypeptide or fusion protein is driven by an inducible promoter. In some embodiments, the inducible promoter is an oxygen level-dependent promoter (e.g., FNR-inducible promoter). In some embodiments, the inducible promoter is induced by gut-specific metabolite and/or a metabolite specific to a disease state, such as PA and/or MMA, or promoters induced by inflammation or an inflammatory response (RNS, ROS promoters), or promoters induced by a metabolite that may or may not be naturally present (e.g., can be exogenously added) in the gut, e.g., arabinose. In some embodiments, the inducible promoter is induced under in vitro strain culture conditions, e.g., expansion, production and/or manufacture, such as the in the presence of arabinose and chemical and/or nutritional inducers described herein. In alternate embodiments, expression of the surface displayed polypeptides or polypeptide fusion proteins is driven by a constitutive promoter, which is active in vivo, e.g., in the gut, in a disease state, such as PA and/or MMA and/or under in vitro strain culture conditions. In some embodiments, the expression of the surface displayed polypeptide or fusion protein is plasmid based. In some embodiments, the gene sequence(s) encoding the antibodies or scFv fragments for surface display is chromosomally inserted.

Table 26 lists polypeptide sequences of exemplary display anchors of the disclosure.

TABLE 26
Selected display anchors

| | | |
|---|---|---|
| SEQ | Invasin | MVFQPISEFLLIRNAGMSMYFNKIISFNIISRIVICIFLICGMFMAGASEKYDANAPQQV |
| ID | display tag | QPYSVSSSAFENLHPNNEMESSINPFSASDTERNAAIIDRANKEQETEAVNKMISTGARL |
| NO: | | AASGRASDVAHSMVGDAVNQEIKQWLNRFGTAQVNLNFDKNFSLKESSLDWLAPWYDSAS |
| 309 | | FLFFSQLGIRNKDSRNTLNLGVGIRTLENGWLYGLNTFYDNDLTGHNHRIGLGAEAWTDY |
| | | LQLAANGYFRLNGWHSSRDFSDYKERPATGGDLRANAYLPALPQLGGKLMYEQYTGERVA |
| | | LFGKDNLQRNPYAVTAGINYTPVPLLTVGVDQRMGKSSKHETQWNLQMNYRLGESFQSQL |
| | | SPSAVAGTRLLAESRYNLVDRNNNIVLEYQKQQVVKLTLSPATISGLPGQVYQVNAQVQG |
| | | ASAVREIVWSDAELIAAGGTLTPLSTTQFNLVLPPYKRTAQVSRVTDDLTANFYSLSALA |
| | | VDHQGNRSNSFTLSVTVQQPQLTLTAAVIGDGAPANGKTAITVEFTVADFEGKPLAGQEV |
| | | VITTNNGALPNKITEKTDANGVARIALTNTTDGVTVVTAEVEGQRQSVDTHFVKGTIAAD |
| | | KSTLAAV |
| SEQ | LppOmpA | KATKLVLGAVILGSTLLAGCSSNAKIDQGINPYVGFEMGYDWLGRMPYKGSVENGAYKAQ |
| ID | display tag | GVQLTAKLGYPITDDLDIYTRLGGMVWRADTKSNVYGKNHDTGVSPVFAGGVEYAITPEI |
| NO: | | ATRLEYQWTNNIGDAHTIGTRPDNGIPG |
| 310 | | |
| SEQ | IntiminN | ITHGCYTRTRHKHKLKKTLIMLSAGLGLFFYVNQNSFANGENYFKLGSDSKLLTHDSYQN |
| ID | display tag | RLFYTLKTGETVADLSKSQDINLSTIWSLNKHLYSSESEMMKAAPGQQIILPLKKLPFEY |
| NO: | | SALPLLGSAPLVAAGGVAGHTNKLTKMSPDVTKSNMTDDKALNYAAQQAASLGSQLQSRS |
| 311 | | LNGDYAKDTALGIAGNQASSQLQAWLQHYGTAEVNLQSGNNFDGSSLDFLLPFYDSEKML |
| | | AFGQVGARYIDSRFTANLGAGQRFFLPANMLGYNVFIDQDFSGDNTRLGIGGEYWRDYFK |
| | | SSVNGYFRMSGWHESYNKKDYDERPANGFDIRFNGYLPSYPALGAKLIYEQYYGDNVALF |
| | | NSDKLQSNPGAATVGVNYTPIPLVTMGIDYRHGTGNENDLLYSMQFRYQFDKSWSQQIEP |
| | | QYVNELRTLSGSRYDLVQRNNNILLEYKKQDILSLNIPHDINGTEHSTQKIQLIVKSKYG |
| | | LDRIVWDDSALRSQGGQIQHSGSQSAQDYQAILPAYVQGGSNIYKVTARAYYRNGNSSNN |
| | | VQLTITVLSNGQVVDQVGVTDFTADKTSAKADNADTITYTATVKKNGVAQANVPVSFNIV |
| | | [02] SGTATLGANSAKTDANGKATVTLKSSTPGQVVVSAKTAEMTSALNASAVIF |
| | | FDQTKAS |

In Vivo Models

The engineered bacteria may be evaluated in vivo, e.g., in an animal model. Any suitable animal model of a disease or condition associated with catabolism of propionate may be used. For example, a hypomorphic mouse model of propionic acidemia as described by Guenzel et al. can be used (see, for example, Guenzel et al., 2013, Molecular. Ther., 21(7):1316-1323). This PCCA−/− knock-out mouse lacks Pcca protein and accumulates high levels of propionylcarnitine and methyl citrate and dies within 36 hours of birth. However, the hypomorphic mouse of PCCA−/− (A138T) survives with elevated levels of propionic acidemia and hence it is a great model to use. Intravenous injections of adeno-associated virus 2/8 (AAV8) vectors to these hypomorphic mice reduced propionylcarnitine and methyl citrate and mediated long lasting effects. A PCCA−/− knock-out mouse model can also be used (see, for example, Miyazaki et al., 2001, J. Biol. Chem., 276:35995-35999). A mouse model of Methylmalonic Acidemia has also been described by Peters et al. (see, for example, Peters et al., 2012, PLoS ONE, 7(7):e40609).

A mouse model of methylmalonic acidemia has been generated by targeted deletion of a critical exon in the murine methylmalonyl-CoA mutase (Mut) gene (VENDITTI C P, et al/. Genetic and genomic systems to study methylmalonic acidemia (MMA) Mol Genet Metab. 2005; 84:207-208). The Mut knockout (KO) model resulted in neonatal lethality of all homozygous (KO/KO) pups. The Mut−/− mice display early neonatal lethality on C57BL/6 background and faithfully replicate the severe phenotype of affected humans. Studies in the Mut−/− mice have demonstrated progressive hepatic pathology and massive accumulation of methylmalonic acid in the liver near the time of death. Next, a Mut-KO mouse on the modified RC57BL/6×129Sv/Ev)×FVB/N1 background was generated, which resulted in some KO mice surviving the neonatal period (Chandler, et al. (2009) Mitochondrial dysfunction in mut methylmalonic acidemia. The FASEB official publication of the Federation of American Societies for Experimental Biology 23, 1252-1261), although nearly all died within 25 days (Chandler and Venditti (2010) Long-term rescue of a lethal murine model of methylmalonic acidemia using adeno-associated viral gene therapy. Molecular therapy: the journal of the American Society of Gene Therapy 18, 11-16). Using this KO model, they applied adeno-associated virus-mediated gene therapy (Chandler and Venditti (2008) Adenovirus-mediated gene delivery rescues a neonatal lethal murine model of mut(0) methylmalonic acidemia. Human gene therapy 19, 53-60). This model has been extensively used to examine the effectiveness of rAAVs in the treatment of MMA. For example, a serotype 9 rAAV expressing the Mut cDNA effectively rescued the Mut−/− mice from lethality, conferred long-term survival, markedly improved metabolism and resulted in striking preservation of renal function and histology (Senac et al., Gene therapy in a murine model of Methylmalonic Acidemia (MMA) using rAAV9 mediated gene delivery; Gene Ther. 2012 April; 19(4): 385-391). Another Mut (−/−) mouse has been described by Peters et al. (Peters et al., A knock-out mouse model for methylmalonic aciduria resulting in neonatal lethality; J Biol Chem. 2003 Dec. 26; 278(52):52909-13 and also Peters et al., 2012, PLoS ONE, 7(7):e40609).

A number of transgenic approaches were also used in attempt to generate MMA models with greater survival rates. Stable transgenic Mut expression restricted to the liver resulted in a long-term rescue of lethality (Manoli, et al. (2013) Targeting proximal tubule mitochondrial dysfunction attenuates the renal disease of methylmalonic acidemia. Proc Natl Acad Sci USA. 2013 Aug. 13; 110(33):13552-7).

To create another model which is less severe, so that long-term effects of methylmalonic acidemia may be studied, the mut−/− could be modified. For example, overexpression of a well-characterized mutant or synthetic MCM allele via a transgenic construct (either as a BAC or transgene driven by a heterologous promoter), may rescue the lethal phenotype of the mut−/− KO models. Alternatively, transgenic rescue with a wild-type gene under control of an inducible promoter or a tissue-specific promoter may be useful in creating a conditional-on model to study the effects of PA and/or MMA on certain organs. Conditional-off alleles also be useful examine the effects of administration of the genetically engineered bacteria on specific organs. In another approach, knocking-in of selected human mutation(s) into the MCM locus, such as those that participate in interallelic complementation or that have predominantly cobalamin Km effects may allow for a versatile model of a partial deficiency to be developed (Chandler and Venditti, 2005; Genetic and genomic systems to study methylmalonic acidemia; Molecular Genetics and Metabolism 86: 34-35, the contents of which is herein incorporated by reference in its entirety). Any such models can be used to study the efficacy and pharmacokinetic properties of the genetically engineered bacteria.

For example, mice with knock in of a Mut allele found in human patients developed by Forny et al. may be used for these studies (Forny et al., Novel Mouse Models of Methylmalonic Aciduria Recapitulate Phenotypic Traits with a Genetic Dosage Effect, J Biol Chem. 2016 Sep. 23; 291(39):20563-73, the contents of which is herein incorporated by reference in its entirety). In this study, the human missense mutation p.Met700Lys (c.2009T>A) (p.Met700Lys in mouse) was knocked into the Mut locus. This mutation was selected due to its residual enzymatic activity and in vitro response to hydroxocobalamin. This constitutive KI allele causes Mut deficiency, which was further aggravated by crossing this knock in with the Mut−/− mice to get a Mutko/ki mouse.

Under normal dietary conditions, kidney dysfunction (increased plasma urea, impaired diuresis, changes in the urinary excretion of electrolytes) and neurotoxicity (increased brain weight, indicating cytotoxic edema) were observed, both of which are also found in MMA patients. Levels of metabolites observed were consistent with those seen in patients. One key phenotypic sign in both Mut ki/ki and MutKi/ko strains was growth retardation (without reduction in food intake) which likely correlates with failure to thrive in human patients. A high protein challenge with both high protein or pre-cursor enriched diet (comprising increased levels of precursor amino acids of propionate pathway metabolites, i.e., threonine, isoleucine leucine, valine) in these models lead to metabolic crisis, manifested substantial elevation of metabolites (C3:C2 in blood, MMA in urine, MMA in blood, ammonia, glycine in blood; fatty acid levels (C13, C14, C15, C16, C17, C18) in plasma, sphingoid bases (C16, C17, C18, C19) in plasma, C17 sphingoid base in tissue) and immediate weight loss in both strains. This situation is consistent with acute metabolic crisis in humans. Metabolic crisis was partially rescued by cobalamin. The KI allele resulted in a milder phenotype than the KO allele, which displayed higher concentrations of MMA, 2MC and C3, more pronounced growth retardation, and a stronger response to the dietary challenge, in analogy to phenotypic differences observed in patients. As such, this model biochemically and clinically models the symptoms of MMA in patients and is a therefore a useful tool to study the efficacy of the genetically engineered bacteria.

The engineered bacterial cells may be administered to the animal, e.g., by oral gavage, and treatment efficacy is determined, e.g., by measuring blood levels of propionylcarnitine, acetylcarnitine, and/or methylcitrate before and after treatment (see, for example, Guenzel et al., 2013). The animal may be sacrificed, and tissue samples may be collected and analyzed. A decrease in blood levels of propionylcarnitine, acetylcarnitine, and/or methylcitrate after treatment indicates that the engineered bacteria are effective for treating the disease. Blood and/or urine levels of methylmalonate may also be measured, and indicate that the engineered bacteria are effective for reducing methylmalonate, e.g., in a model of MMA. Other markers described herein, including but limited to C16, C17, C4DC, also can be measured.

Methods of Screening

Generation of Bacterial Strains with Enhance Ability to Transport Metabolites or Biomolecules

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite or biomolecule.

For example, if the biosynthetic pathway for producing a certain metabolite or biomolecule is disrupted a strain capable of high-affinity capture of said metabolite or biomolecule can be evolved via ALE. First, the strain is grown in varying concentrations of the auxotrophic amino acid or metabolite, until a minimum concentration to support growth is established. The strain is then passaged at that concentration, and diluted into lowering concentrations of the metabolite or biomolecule at regular intervals. Over time, cells that are most competitive for the metabolite or biomolecule—at growth-limiting concentrations—will come to dominate the population. These strains will likely have mutations in their metabolite-transporters resulting in increased ability to import the essential and limiting metabolite or biomolecule.

Similarly, by using an auxotroph that cannot use an upstream metabolite to form a certain metabolite or biomolecule, a strain can be evolved that not only can more efficiently imports the upstream metabolite, but also converts the metabolite into the essential downstream metabolite. These strains will also evolve mutations to increase import of the upstream metabolite, but may also contain mutations which increase expression or reaction kinetics of downstream enzymes, or that reduce competitive substrate utilization pathways.

A metabolite innate to the microbe can be made essential via mutational auxotrophy and selection applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound, this gene can be recombinantly introduced and expressed in the heterologous host. This new host strain will now have the ability to synthesize an essential nutrient from a previously non-metabolizable substrate.

Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 1011.2 CCD1. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

Similar methods can be used to generate E. coli Nissle mutants that consume or import propionate and or one or more of its metabolites.

Pharmaceutical Compositions and Formulations

Pharmaceutical compositions comprising the genetically engineered bacteria described herein may be used to treat, manage, ameliorate, and/or prevent disorders associated with propionate catabolism. Pharmaceutical compositions comprising one or more genetically engineered bacteria, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., to express at least one propionate catabolism gene or gene cassette. In alternate embodiments, the pharmaceutical composition comprises two or more species, strains, and/or subtypes of bacteria that are each engineered to comprise the genetic modifications described herein, e.g., to express at least one propionate catabolism gene(s) or gene cassette(s). In some embodiments, the pharmaceutical composition may comprise one or more bacterial strains comprising circuitry for the consumption of ammonium and optionally one or more ammonium transporter(s)/importer(s) and/or arginine exporter(s), as described in co-owned U.S. Pat. No. 9,487,764 and US Patent Publication No. US20160177274, the contents of each of which is herein incorporated by reference in their entireties. Any of the strains described in U.S. Pat. No. 9,487,764 and US Patent Publication No. US20160177274 are useful for the reduction of ammonia levels in a subject, i.e., for the treatment of hyperammonemia, e.g., as is observed in PA and MMA patients. Any of the strains described in U.S. Pat. No. 9,487,764 and US Patent Publication No. US20160177274 can be used alone or in combination with one or more strains for the reduction of propionate and/or methylmalonate, as described herein for the treatment of PA and/or MMA in a subject. In some embodiments, the pharmaceutical composition comprises one or more bacterial strains comprising circuitry for the catabolism of branched chain amino acids (BCAA) (e.g., leucine, isoleucine, and/or valine) and optionally one or more BCAA transporter(s)/importer(s) and/or related metabolite exporter(s), as described in co-owned International Patent Application No. PCT/US2016/037098, the contents of which is herein incorporated by reference in its entirety. Such strains prevent or reduce the production of acetoacetate, acetyl-CoA, propionyl-CoA, and/or propionate from leucine, isoleucine, and/or valine and are therefore useful in the reduction of propionate and/or methylmalonate levels. Any of the strains described in International Patent Application No. PCT/US2016/037098 can be used alone or in combination with one or more strains for the reduction of propionate and/or methylmalonate, as described herein, or the treatment of PA and/or MMA in a subject.

In some embodiments three types of genetically engineered strains are administered in combination in the pharmaceutical composition, e.g., one or more strains of a for the catabolism of propionate, described herein, one or more strains for the consumption of ammonium, as described in U.S. Pat. No. 9,487,764 and US Patent Publication No. US20160177274, and one or more strains for the catabolism of branched chain amino acids as described in International Patent Application No. PCT/US2016/037098.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered bacteria described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, immediate-release, pulsatile-release, delayed-release, or sustained release). Suitable dosage amounts for the genetically engineered bacteria may range from about 10⁵ to 10¹² bacteria, e.g., approximately 10⁵ bacteria, approximately 10⁶ bacteria, approximately 10⁷ bacteria, approximately 10⁸ bacteria, approximately 10⁹ bacteria, approximately 10¹⁰ bacteria, approximately 10¹¹ bacteria, or approximately 10¹¹ bacteria. The composition may be administered once or more daily, weekly, or monthly.

The composition may be administered before, during, or following a meal. In one embodiment, the pharmaceutical composition is administered before the subject eats a meal. In one embodiment, the pharmaceutical composition is administered currently with a meal. In one embodiment, the pharmaceutical composition is administered after the subject eats a meal.

The genetically engineered bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The genetically engineered bacteria disclosed herein may be administered topically and formulated in the form of an ointment, cream, transdermal patch, lotion, gel, shampoo, spray, aerosol, solution, emulsion, or other form well-known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the engineered bacteria may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered bacteria disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered bacteria are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionized at low pH, and therefore remain insoluble. But as the pH increases in the gastrointestinal tract, the acidic functional groups are capable of ionization, and the polymer swells or becomes soluble in the intestinal fluid.

Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Coating polymers also may comprise one or more of, phthalate derivatives, CAT, HPMCAS, polyacrylic acid derivatives, copolymers comprising acrylic acid and at least one acrylic acid ester, Eudragit™ S (poly(methacrylic acid, methyl methacrylate)1:2); Eudragit L100™ S (poly(methacrylic acid, methyl methacrylate)1:1); Eudragit L30D™, (poly(methacrylic acid, ethyl acrylate)1:1); and (Eudragit L100-55) (poly(methacrylic acid, ethyl acrylate)1:1) (Eudragit™ L is an anionic polymer synthesized from methacrylic acid and methacrylic acid methyl ester), polymethyl methacrylate blended with acrylic acid and acrylic ester copolymers, alginic acid, ammonia alginate, sodium, potassium, magnesium or calcium alginate, vinyl acetate copolymers, polyvinyl acetate 30D (30% dispersion in water), a neutral methacrylic ester comprising poly(dimethylaminoethylacrylate) (“Eudragit E™), a copolymer of methylmethacrylate and ethylacrylate with trimethylammonioethyl methacrylate chloride, a copolymer of methylmethacrylate and ethylacrylate, Zein, shellac, gums, or polysaccharides, or a combination thereof.

Coating layers may also include polymers which contain Hydroxypropylmethylcellulose (HPMC), Hydroxypropylethylcellulose (HPEC), Hydroxypropylcellulose (HPC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), methylhydroxyethylcellulose (M H EC), hydrophobically modified hydroxyethylcellulose (NEXTON), carboxymethyl hydroxyethylcellulose (CMHEC), Methylcellulose, Ethylcellulose, water soluble vinyl acetate copolymers, gums, polysaccharides such as alginic acid and alginates such as ammonia alginate, sodium alginate, potassium alginate, acid phthalate of carbohydrates, amylose acetate phthalate, cellulose acetate phthalate (CAP), cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS).

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered bacteria described herein.

In one embodiment, the genetically engineered bacteria of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered bacteria may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the engineered bacteria may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the engineered bacteria are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the engineered bacteria are well known in the art. For example, see U.S. 2015/0359894 and US 2015/0238545, the entire contents of each of which are expressly incorporated herein by reference. In yet another embodiment, the pharmaceutical composition is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered bacteria described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered bacteria may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans. The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

Methods of Treatment

Another aspect of the disclosure provides methods of treating a disease associated with catabolism of propionate in a subject, or symptom(s) associated with the disease associated with the catabolism of propionate in a subject. In one embodiment, the disorder involving the catabolism of propionate is a metabolic disorder involving the abnormal catabolism of propionate. Metabolic diseases associated with abnormal catabolism of propionate include propionic acidemia (PA) and methylmalonic acidemia (MMA), as well as severe nutritional vitamin B₁₂ deficiencies. In one embodiment, the disease associated with abnormal catabolism of propionate is propionic acidemia. In one embodiment, the disease associated with abnormal catabolism of propionate is methylmalonic acidemia. In another embodiment, the disease associated with abnormal catabolism of propionate is a vitamin B₁₂ deficiency.

In one embodiment, the disease is propionic acidemia. Propionic acidemia, also known as propionyl-CoA carboxylase deficiency, PROP, PCC deficiency, ketotic hyperglycinemia, ketotic glycinemia, and hyper glycinemia with ketoacidosis and leukopenia, is an autosomal recessive disorder caused by impaired activity of Propionyl CoA carboxylase (PCC; EC 6.4.1.3). PCC is responsible for converting propionyl CoA into methylmalonyl CoA. Patients with PA are unable to properly process propionyl CoA, which can lead to the toxic accumulation of propionyl CoA and propionic acid in the blood, cerebrospinal fluid and tissues. Clinical manifestations of the disease vary depending on the degree of enzyme deficiency and include seizures, vomiting, lethargy, hypotonia, encephalopathy, developmental delay, failure to thrive, and secondary hyperammonemia (Deodato et al., Methylmalonic and propionic aciduria, Am. J. Med. Genet. C. Semin. Med. Genet, 142(2):104-112, 2006).

Propionyl CoA Carboxylase (PCC) is a dodecameric enzyme comprised of alpha and beta subunits. The alpha subunit of PCC (also called PCCA; NM_000282) comprises the biotin carboxylase and biotin carboxyl carrier protein domains, while the beta subunit (also called PCCB; NM_000532) contains the carboxyltransferase activity (Diacovich et al., Biochemistry, 43(44):14027-14036, 2004). Mutations in either the PPCA or PPCB genes can lead to the development of Propionic Acidemia, and more than twenty-four mutations in genes encoding PCCA or PCCB have been identified that result in Propionic Acidemia (Perez et al., Mol. Genet Metabol., 78(1):59-67, 2003), including missense mutations, nonsense mutations, point exonic mutations affecting splicing, splicing mutations, insertions and deletions.

Because of the inability to properly breakdown amino acids completely, patients having a disease associated with catabolism of propionate accumulate different byproduct molecules in their blood and urine (Carrillo-Carrasco and Venditti, Gene Reviews. Seattle (Wash.): University of Washington, Seattle; 1993-2015). The abnormal levels of these by-product molecules are used as the main diagnostic criteria for diagnosing the disorder (See, e.g., Table 27).

TABLE 27
Breakdown Products of Propionate

| | Normal Values | LC-MS/MS method |
|---|---|---|
| Blood metabolite | | |
| Propionylcarnitine | | Yes |
| Methylcitrate | | Yes |
| Glycine | | Yes |
| Propionate | | Yes (in vitro assay) |
| Urine metabolite | | |
| 3-hydroxypropionate | 3-10 mmol/mol Cr | No |
| Methylcitrate | Normally absent | Yes |
| Tiglylglycine | Normally absent | No |
| Propionylglycine | Normally absent | No |
| Lactate (occasionally) | | |

Detectable urinary organic acids useful for diagnosis and markers include, but are not limited to, N-propionylglycine, N-tiglyglycine, 2-methyl-3-oxovaleric acid, 3-hydroxy-2-methylbutyric acid, 2 methyl-3-oxobutyric acid, 3-hydroxy-n-valeric acid, 3-oxo-n-valeric acid. Such urinary organic acids are useful in the analysis of treatments with the pharmaceutical compositions comprising the strains, e.g., to determine efficacy, and pharmacokinetics of the compositions.

In one embodiment, the disease is methylmalonic acidemia. Methylmalonic acidemia, also known as methylmalonic aciduria or isolated methylmalonic acidemia, is an autosomal recessive disorder caused by impaired activity of one of several genes: MUT (OMIM 251000), MMAA (OMIM 251100), MMAB (OMIM 251110), MMACHC (OMIM 27740), MMADHC (OMIM 277410), or LMBRD1 (OMIM 277380). However, over sixty percent of subjects with methylmalonic acidemia have mutations in the methylmalonyl CoA mutase (MUT) gene. MUT is responsible for converting methylmalonyl CoA into succinyl CoA and requires a vitamin B₁₂-derived prosthetic group, adenosylcoalamin (also known as AdoCbl) to function. Methylmalonic aciduria of the complementation group ‘mut’ is caused by mutation in the gene encoding methylmalonyl-CoA mutase (MUT; 609058). Upon entry into the mitochondria, the mitochondrial leader sequence at the N-terminus of MUT is cleaved, and MUT monomers then associate into homodimers. The methylmalonic aciduria type A protein, mitochondrial (also known as MMAA) aides AdoCbl loading onto MUT. Methylmalonic aciduria of the cblA complementation type is caused by homozygous or compound heterozygous mutation in the MMAA gene (607481) Similarly, Cob(l)yrinic acid, a,c-diamind adenosyltransferase, mitochondrial (MMAB), is an enzyme that catalyzes the final step in the conversion of vitamin B₁₂ into adenosylcobalamin (AdoCbl). Methylmalonic aciduria of the cblB complementation type is caused by homozygous or compound heterozygous mutation in the MMAB gene (607568) Methylmalonic aciduria and homocystinura type C protein, mitochondrial (also known as MMACHC) and methylmalonic aciduria and homocystinurai type D protein, mitochondrial (also known as MMADHC) encode mitochondrial proteins that are also involved in vitamin B₁₂ (cobalamin) synthesis. CblC type of combined methylmalonic aciduria and homocystinuria is caused by homozygous or compound heterozygous mutation in the MMACHC gene (609831) and methylmalonic aciduria and homocystinuria, isolated homocystinuria, and isolated methylmalonic aciduria of complementation group cblD are all caused by homozygous or compound heterozygous in the MMADHC gene (611935). Methylmalonyl CoA epimerase encodes an enzyme that interconverts D- and L-methylmalonyl-CoA during the degradation of branched-chain amino acids, odd chain-length fatty acids, and other metabolites, homozygous mutation in the MCEE gene (608419) causes methylmalonyl-CoA epimerase deficiency (OMIM:251120), which may result in moderate methylmalonic aciduria.

SUCLA2 gene encodes the beta-subunit of the ADP-forming succinyl-CoA synthetase (SCS-A; EC 6.2.1.5). SCS is a mitochondrial matrix enzyme that catalyzes the reversible synthesis of succinyl-CoA from succinate and CoA. Mitochondrial DNA depletion syndrome-5 (MTDPSS; OMIM: 612073), which shows mild methylmalonic aciduria, is caused by homozygous or compound heterozygous mutation in the beta subunit of the succinate-CoA ligase gene (SUCLA2; 603921). SUCLG1 gene encodes the alpha subunit of mitochondrial succinyl CoA synthetase. Mitochondrial DNA depletion syndrome-9 (MTDPS9) is caused by homozygous or compound heterozygous mutation in the alpha subunit of the succinate-CoA ligase gene (SUCLG1; 611224). Methylmalonic acidemia can also be associated with hyperhomocysteinemia or homocystinuria caused by defects in other steps of intracellular cobalamin metabolism (e.g., as described in Gene Reviews: Disorders of Intracellular Cobalamin Metabolism; Nuria Carrillo, MD, David Adams, MD, PhD, and Charles P Venditti, MD, PhD).

Co-called atypical MMA is associated with increased, usually mild urinary excretion of methylmalonate. Causes of atypical MMA can be sare defects, such as combined malonic and methylmalonic acidemia (CMAMMA) caused by ACSF3 deficiency, methylmalonate semialdehyde dehydrogenase deficiency (MMSDH) caused by mutation of the ALDH6A1 gene, transcobalamin receptor deficiency (TCbIIR/CD320), and combined methylmalonic acidemia and homocysteinemia (caused by mutation in HCFC1).

Patients with MMA are unable to properly process methylmalonyl CoA, which can lead to the toxic accumulation of methylmalonyl CoA and methylmalonic acid in the blood, cerebrospinal fluid and tissues. Clinical manifestations of the disease vary depending on the degree of enzyme deficiency and include seizures, vomiting, lethargy, hypotonia, encephalopathy, developmental delay, failure to thrive, and secondary hyperammonemia (Deodato et al., Methylmalonic and propionic aciduria, Am. J. Med. Genet. C. Semin. Med. Genet, 142(2):104-112, 2006).

In diagnosis of MMA, relevant findings in laboratory tests include high plasma and urine MMA with normal B12, tHcy, and methionine levels; elevated propionylcarnitine (C3); high anion gap metabolic acidosis in arterial or venous blood gas testing and huge quantities of ketone bodies and lactate in the urine; hyperammonemia; hyperglycinemia; lactic acidosis; complete blood chemistry showing neutropenia, thrombocytopenia, and anemia as described in GeneReviews Manoli et al., Isolated Methylmalonic Acidemia and references therein).

Table 28 shows levels of methylmalonic acid in various subtypes od methylmalonic acidemia (as described in GeneReviews Manoli et al., Isolated Methylmalonic Acidemia and references therein).

TABLE 28
Levels of methylmalonic acid in various subtypes od methylmalonic
acidemia

| Methylmalonic Acidemia | Methylmalonic Acid Concentration | |
|---|---|---|
| Phenotype/Enzymatic Subtype | Urine² | Blood |

| | | | | |
|---|---|---|---|---|
| Infantile/non-B₁₂-responsive | 1000-10,000 | mmol/mol Cr | 100-1000 | μM |
| mut⁰, mut⁻, cblB | | | | |
| B₁₂-responsive cblA, cblD-MMA | Tens-hundreds | mmol/mol Cr | 5-100 | μM |
| cblB, mut⁻ (rare) | | | | |
| “Benign”/adult methylmalonic acidemia | 10-100 | mmol/mol Cr | 100 | μM |
| Methylmalonyl-CoA epimerase (MCEE) | 50-1,500 | mmol/mol Cr | 7 | μM |
| deficiency | | | | |
| Normal | <4 | mmol/mol Cr⁷ | <0.27 | μM⁷ |

In addition to elevated methylmalonic acid (e.g., detected by urine or blood analysis) and altered plasma acylcarnitine profile, elevated 3-hydroxypropionate, 2-methylcitrate, and tiglylglycine may be detected in the urine. Elevated plasma concentrations of glycine (on plasma amino acid analysis) and elevated plasma concentration of propionylcarnitine (C3) and variable elevations in C4-dicarboxylic or methylmalonic/succinylcarnitine (C4DC), e.g., measured by TMS, may be observed. Elevated C4-dicarboxylic acylcarnitine (C4DC) is considered a marker indicative of MMA associated with succinyl-CoA ligase deficiency, as its accumulation can result from methylmalonylcarnitine and succinylcarnitine.

The acylcarnitine profile of dried blood spot (DBS) samples from newborns with a propionate metabolism defect usually shows increased levels of propionylcarnitine (C3). In order to improve the specificity and sensitivity, it has been suggested to include the calculation of the metabolite ratios C3/C2, C3/C16, C3/C17, and C3/Met in the newborn screening panel and using pattern recognition algorithms Additionally, second trier tests have been developed, for example one 2^{nd} tier test measures the presence of 3-OH-propionic or methylmalonic acids on the same dried blood spot. More recently, new biomarkers such as 3-hydroxypalmitoleoyl-carnitine (C16:1OH) have been employed in combination with high blood concentration of C3 to determine a positive test result in newborn screening, in combination with acylcarnitine analysis by MS/MS. This marker can be used for both for MMA and PA. C16:1-OH and other hydroxylated long chain acylcarnitines are well-known markers of long-chain 3-hydroxyacyl-CoA dehydrogenase deficiency (LCHADD) and/or trifunctional protein (TFP) deficiency. It has also been suggested that a new metabolite, C17 acylcarnitine, can be used as a primary diagnostic tool for the diagnosis of propionate metabolism defects (both MMA and PA) and should be considered an important biomarker (Malvagia et al., Heptadecanoylcarnitine (C17) a novel candidate biomarker for newborn screening of propionic and methylmalonic acidemias; Clin Chim Acta. 2015 Oct. 23; 450:342-8).

As such, measurement of these metabolites can provide a useful to determine the efficacy and pharmacokinetics of the genetically engineered bacteria as they are administered to a subject, e.g., for the treatment of MMA. Reduction is measured by comparing the levels and ratios of these metabolites in a subject before and after administration of the pharmaceutical composition comprising the genetically engineered bacteria.

Currently available treatments for Propionic Acidemia and Methylmalonic Acidemia are inadequate for the long-term management of the disease and have severe limitations (Li et al., Liver Transplantation, 2015). A low protein diet, with micronutrient and vitamin supplementation, as necessary, is the widely accepted long-term disease management strategy for PA and MMA (Li et al., 2015).

To avoid excessive propiogenic amino acid load (isoleucine, valine, methionine and threonine) into the pathway, a propiogenic amino acid-deficient formula (e.g., Propimex®-1/2, XMTVI-1/2, OA-1/2) and protein-free formula (e.g., Pro-Phree®, Duocal®) are given to some infants to provide extra fluid and calories.

However, protein-intake restrictions can be particularly problematic and result in significant morbidity. Even with proper monitoring and patient compliance, protein dietary restrictions result in a high incidence of mental retardation and mortality (Li et al., 2015). Additional non-surgical chronic management regimens include L carnitine administration. Carnitine can be given at a dose of 50-100 mg/kg/day, up to approximately 300 mg/kg/day. As a dietary supplement, carnitine may replace the free carnitine pool and enhance the conjugation and excretion of propionylcarnitine. Antibiotics (e.g., metronidazole 10-15 mg/kg/day or Oral neomycin, 250 mg by mouth 4×/day), to reduce the production of propionate from gut flora can be used.

Vit B12 is suggested for select MMA responsive patients (cblA>cblB>mut (−)); e.g., through hydroxocobalamin injections (1.0-mg injections every day to every other day are usually required in individuals who are vitamin B12 responsive). The regimen of B12 injections needs to be individually adjusted according to the patient's age and, possibly, weight.

Other options include antioxidants, coenzyme Q10 and vitamin E, amino acid dietary formulas (isoleucine/valine, glutamine, alanine supplementation), and dialysis. Further, a few cases of PA and MMA have been treated by liver transplantation (Li et al., 2015), kidney transplantation or combined liver/kidney transplantation. However, the limited availability of donor organs, the costs associated with the transplantation itself, and the undesirable effects associated with continued immunosuppressant therapy limit the practicality of liver transplantation for treatment of disease. Therefore, there is significant unmet need for effective, reliable, and/or long-term treatment for PA and MMA.

The present disclosure surprisingly demonstrates that pharmaceutical compositions comprising the engineered bacterial cells may be used to treat metabolic diseases involving the abnormal catabolism of propionate, such as PA and MMA.

In one embodiment, the subject having PA has a mutation in a PCCA gene. In another embodiment, the subject having PA has a mutation in the PCCB gene.

In one embodiment, the subject having MMA has a mutation in the MUT gene. In another embodiment, the subject having MMA has a mutation in the MMAA gene. In another embodiment, the subject having MMA has a mutation in the MMAB gene. In another embodiment, the subject having MMA has a mutation in the MMACHC gene. In another embodiment, the subject having MMA has a mutation in the MMADHC gene. In another embodiment, the subject having MMA has a mutation in the LMBRD1 gene. In another embodiment, the subject having MMA has a mutation in the ACSF3 gene. In another embodiment, the subject having MMA has a mutation in the SUCLA2 gene. In another embodiment, the subject having MMA has a mutation in the SUCLG1 gene. In another embodiment, the subject having MMA has a mutation in the ALDH6A1 gene. In another embodiment, the subject having MMA has a mutation in the HCFC1 gene.

In another aspect, the disclosure provides methods for decreasing the plasma level of propionate, propionyl CoA, and/or methylmalonic CoA in a subject by administering a pharmaceutical composition comprising a bacterial cell to the subject, thereby decreasing the plasma level of the propionate, propionyl CoA, and/or methylmalonic CoA in the subject. In one embodiment, the subject has a disease or disorder involving the catabolism of propionate. In one embodiment, the disorder involving the catabolism of propionate is a metabolic disorder involving the abnormal catabolism of propionate. In another embodiment, the disorder involving the catabolism of propionate is propionic acidemia. In another embodiment, the disorder involving the catabolism of propionate is methylmalonic acidemia. In another embodiment, the disorder involving the catabolism of propionate is a vitamin B₁₂ deficiency.

In some embodiments, the disclosure provides methods for reducing, ameliorating, or eliminating one or more symptom(s) associated with these diseases, including but not limited to seizures, vomiting, lethargy, hypotonia, encephalopathy, developmental delay, failure to thrive, liver failure, and/or secondary hyperammonemia. In some embodiments, the disease is secondary to other conditions, e.g., liver disease. In some embodiments, the disclosure provides methods for reducing, ameliorating, or eliminating one or more symptom(s) associated with these diseases, intellectual disability, tubulointerstitial nephritis with progressive impairment of renal function, “metabolic stroke” or infarction of the basal ganglia, pancreatitis, growth failure, functional immune impairment, bone marrow failure, optic nerve atrophy, and hepatoblastoma.

In certain embodiments, the bacterial cells are capable of catabolizing propionate, propionyl CoA, methylmalonate and/or methylmalonyl CoA in a subject in order to treat a disease associated with catabolism of propionate. In some embodiments, the bacterial cells are delivered simultaneously with dietary protein. In another embodiment, the bacterial cells are delivered simultaneously with L-carnitine. In some embodiments, the bacterial cells and dietary protein are delivered after a period of fasting or protein-restricted dieting. In these embodiments, a patient suffering from a disorder involving the catabolism of propionate, e.g., PA or MMA, may be able to resume a substantially normal diet, or a diet that is less restrictive than a protein-free or very low-protein diet. In some embodiments, the bacterial cells may be capable of catabolizing propionate, propionyl CoA, methylmalonate, and/or methylmalonyl CoA from additional sources, e.g., the blood, in order to treat a disease associated with the catabolism of propionate. In these embodiments, the bacterial cells need not be delivered simultaneously with dietary protein, and a gradient is generated, e.g., from blood to gut, and the engineered bacteria catabolize the propionate, propionyl CoA, methylmalonate, and/or methylmalonyl CoA and reduce plasma levels of the propionate, propionyl CoA, methylmalonate, and/or methylmalonyl CoA, as well as other metabolites. Such other metabolites which are reduced in the plasma and/or urine include propionate, methylmalonic acid, propionylcarnitine (C3), 2-hydroxypropionate, 2-methylcitrate, and tiglylglycine, glycine, C4-dicarboxylic or methylmalonic/succinylcarnitine (C4DC), hydroxypalmitoleoyl-carnitine (C16:1-OH), Heptadecanoylcarnitine (C17). Additionally, metabolite ratios C3/C2, C3/C16, C3/C17, and C3/Met in the subject are modulated.

The method may comprise preparing a pharmaceutical composition with one or more genetically engineered species, strain, or subtype of bacteria described herein, and administering the pharmaceutical composition to a subject in a therapeutically effective amount. In some embodiments, the genetically engineered bacteria disclosed herein are administered orally, e.g., in a liquid suspension. In some embodiments, the genetically engineered bacteria are lyophilized in a gel cap and administered orally. In some embodiments, the genetically engineered bacteria are administered via a feeding tube or gastric shunt. In some embodiments, the genetically engineered bacteria are administered rectally, e.g., by enema. In some embodiments, the genetically engineered bacteria are administered topically, intraintestinally, intrajejunally, intraduodenally, intraileally, and/or intracolically.

In certain embodiments, the pharmaceutical composition described herein is administered to reduce propionate, propionyl CoA, methylmalonate, and/or methylmalonyl CoA levels in a subject. In some embodiments, the methods of the present disclosure reduce the propionate, propionyl CoA, methylmalonate, and/or methylmalonyl CoA levels in a subject by at least about 10%, 20%, 25%, 30%, 40%, 50%, 60%, 70%, 75%, 80%, 85%, 90%, 95%, or more. In another embodiment, the methods of the present disclosure reduce the propionate, propionyl CoA, methylmalonate, and/or methylmalonyl CoA levels in a subject by at least two-fold, three-fold, four-fold, five-fold, six-fold, seven-fold, eight-fold, nine-fold, or ten-fold. In some embodiments, reduction is measured by comparing the propionate, propionyl CoA, methylmalonate, and/or methylmalonyl CoA level in a subject before and after administration of the pharmaceutical composition. In one embodiment, the propionate, propionyl CoA, methylmalonate, and/or methylmalonyl CoA level is reduced in the gut of the subject. In another embodiment, the propionate, propionyl CoA, methylmalonate, and/or methylmalonyl CoA level is reduced in the blood of the subject. In another embodiment, the propionate, propionyl CoA, methylmalonate, and/or methylmalonyl CoA level is reduced in the plasma of the subject. In another embodiment, the propionate, propionyl CoA, methylmalonate, and/or methylmalonyl CoA level is reduced in the brain of the subject.

In one embodiment, the pharmaceutical composition described herein is administered to reduce propionate, propionyl CoA, methylmalonate, and/or methylmalonyl CoA levels in a subject to normal levels. In another embodiment, the pharmaceutical composition described herein is administered to reduce propionate, propionyl CoA, methylmalonate, and/or methylmalonyl CoA levels in a subject to below a normal level.

In some embodiments, the method of treating the disorder involving the catabolism of propionate, e.g., PA or MMA, allows one or more symptoms of the condition or disorder to improve by at least about 10%, 20%, 30%, 40%, 50%, 60%, 70%, 80%, 90%, 95%, or more. In some embodiments, the method of treating the disorder involving the catabolism of propionate, e.g., PA or MMA, allows one or more symptoms of the condition or disorder to improve by at least about two-fold, three-fold, four-fold, five-fold, six-fold, seven-fold, eight-fold, nine-fold, or ten-fold.

Metabolite levels, e.g., propionate, methylmalonic acid, propionylcarnitine (C3), 2-hydroxypropionate, 2-methylcitrate, and tiglylglycine, glycine, C4-dicarboxylic or methylmalonic/succinylcarnitine (C4DC), hydroxypalmitoleoyl-carnitine (C16:1-OH), Heptadecanoylcarnitine (C17), the metabolite ratios C3/C2, C3/C16, C3/C17, and C3/Met in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods described herein may include administration of the compositions of the disclosure to reduce such metabolites and change the ratios of such metabolites. In some embodiments, such metabolites are measured prior to administration of the compositions comprising the genetically engineered bacteria and at certain times after the administration to determine efficacy of the compositions.

In some embodiments, such metabolite measurements in the urine, alone or in combination with blood and plasma metabolite measurements, are used evaluate safety of the pharmaceutical composition of the disclosure in animal models and human subjects. In some embodiments, such metabolite measurements in the urine and/or blood and plasma metabolite measurements, are used in the evaluation of dose-response and optimal regimen for the desired pharmacologic effect and safety of the pharmaceutical composition of the disclosure. In some embodiments, metabolite measurements in the urine and/or blood and plasma metabolite measurements, are used as surrogate endpoint for efficacy and/or toxicity of the pharmaceutical composition of the disclosure. In some embodiments, metabolite measurements in the urine and/or blood and plasma metabolite measurements, are used to predict patients' response to a regimen comprising a therapeutic strain of the pharmaceutical composition of the disclosure. In some embodiments, such metabolite measurements in the urine and/or blood and plasma metabolite measurements, are used for the identification of certain patient populations that are more likely to respond to the drug therapy comprising administration of the pharmaceutical composition of the disclosure. In some embodiments, metabolite measurements in the urine and/or blood and plasma metabolite measurements, are used to avoid specific adverse events. In some embodiments, metabolite measurements in the urine and/or blood and plasma metabolite measurements, are useful for selection of patients which can be treated with the pharmaceutical composition of the disclosure. In some embodiments, metabolite measurements in the urine and/or blood and plasma metabolite measurements, are used as one method for adjusting protein intake/diet of a PA and/or MMA patient on a regimen which includes the administration of the pharmaceutical compositions of the disclosure.

Before, during, and after the administration of the pharmaceutical composition, propionate and/or methylmalonate levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of propionate and/or methylmalonate. In some embodiments, the methods may include administration of the compositions of the disclosure to reduce the propionate and/or methylmalonate to undetectable levels in a subject. In some embodiments, the methods may include administration of the compositions of the disclosure to reduce the propionate and/or methylmalonate concentrations to undetectable levels, or to less than about 1%, 2%, 5%, 10%, 20%, 25%, 30%, 40%, 50%, 60%, 70%, 75%, 80%, 85%, 90%, or 95% of the subject's propionate and/or methylmalonate levels prior to treatment.

In some embodiments, the engineered bacterial cells produce a propionate catabolism enzyme under exogenous environmental conditions, such as the low-oxygen environment of the mammalian gut, to reduce levels of propionate and/or methylmalonate in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, 3-hydroxypropionate, 2-methylcitrate, and/or tiglylglycine levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of 3-hydroxypropionate, 2-methylcitrate, and/or tiglylglycine. In some embodiments, the methods may include administration of the compositions of the disclosure to reduce the 3-hydroxypropionate, 2-methylcitrate, and/or tiglylglycine to undetectable levels in a subject. In some embodiments, the methods may include administration of the compositions of the disclosure to reduce the 3-hydroxypropionate, 2-methylcitrate, and/or tiglylglycine concentrations to undetectable levels, or to less than about 1%, 2%, 5%, 10%, 20%, 25%, 30%, 40%, 50%, 60%, 70%, 75%, 80%, 85%, 90%, or 95% of the subject's 3-hydroxypropionate, 2-methylcitrate, and/or tiglylglycine levels prior to treatment.

In some embodiments, the engineered bacterial cells produce a propionate catabolism enzyme under exogenous environmental conditions, such as the low-oxygen environment of the mammalian gut, to reduce levels of 3-hydroxypropionate, 2-methylcitrate, and/or tiglylglycine in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, glycine levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of glycine. In some embodiments, the methods may include administration of the compositions of the disclosure to reduce the glycine to undetectable levels in a subject. In some embodiments, the methods may include administration of the compositions of the disclosure to reduce the glycine concentrations to undetectable levels, or to less than about 1%, 2%, 5%, 10%, 20%, 25%, 30%, 40%, 50%, 60%, 70%, 75%, 80%, 85%, 90%, or 95% of the subject's glycine levels prior to treatment.

In some embodiments, the engineered bacterial cells produce a propionate catabolism enzyme under exogenous environmental conditions, such as the low-oxygen environment of the mammalian gut, to reduce levels of glycine in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, C4-dicarboxylic acylcarnitine (C4DC) and/or methylmalonylcarnitine and/or succinylcarnitine levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of C4-dicarboxylic acylcarnitine (C4DC) and/or methylmalonylcarnitine and/or succinylcarnitine. In some embodiments, the methods may include administration of the compositions of the disclosure to reduce the C4-dicarboxylic acylcarnitine (C4DC) and/or methylmalonylcarnitine and/or succinylcarnitine to undetectable levels in a subject. In some embodiments, the methods may include administration of the compositions of the disclosure to reduce the C4-dicarboxylic acylcarnitine (C4DC) and/or methylmalonylcarnitine and/or succinylcarnitine concentrations to undetectable levels, or to less than about 1%, 2%, 5%, 10%, 20%, 25%, 30%, 40%, 50%, 60%, 70%, 75%, 80%, 85%, 90%, or 95% of the subject's C4-dicarboxylic acylcarnitine (C4DC) and/or methylmalonylcarnitine and/or succinylcarnitine levels prior to treatment.

In some embodiments, the engineered bacterial cells produce a propionate catabolism enzyme under exogenous environmental conditions, such as the low-oxygen environment of the mammalian gut, to reduce levels of C4-dicarboxylic acylcarnitine (C4DC) and/or methylmalonylcarnitine and/or succinylcarnitine in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, propionylcarnitine (C3)levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of propionylcarnitine. In some embodiments, the methods may include administration of the compositions of the disclosure to reduce the propionylcarnitine to undetectable levels in a subject. In some embodiments, the methods may include administration of the compositions of the disclosure to reduce the propionylcarnitine concentrations to undetectable levels, or to less than about 1%, 2%, 5%, 10%, 20%, 25%, 30%, 40%, 50%, 60%, 70%, 75%, 80%, 85%, 90%, or 95% of the subject's propionylcarnitine levels prior to treatment.

In some embodiments, the engineered bacterial cells produce a propionate catabolism enzyme under exogenous environmental conditions, such as the low-oxygen environment of the mammalian gut, to reduce levels of propionylcarnitine in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, 3-hydroxypalmitoleoyl-carnitine (C16:1OH) levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of C16:1OH. In some embodiments, the methods may include administration of the compositions of the disclosure to reduce the C16:1OH to undetectable levels in a subject. In some embodiments, the methods may include administration of the compositions of the disclosure to reduce the C16:1OH concentrations to undetectable levels, or to less than about 1%, 2%, 5%, 10%, 20%, 25%, 30%, 40%, 50%, 60%, 70%, 75%, 80%, 85%, 90%, or 95% of the subject's C16:1OH levels prior to treatment.

In some embodiments, the engineered bacterial cells produce a propionate catabolism enzyme under exogenous environmental conditions, such as the low-oxygen environment of the mammalian gut, to reduce levels of C16:1OH in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, heptadecanoylcarnitine (C17) levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of C17. In some embodiments, the methods may include administration of the compositions of the disclosure to reduce the C17 to undetectable levels in a subject. In some embodiments, the methods may include administration of the compositions of the disclosure to reduce the C17 concentrations to undetectable levels, or to less than about 1%, 2%, 5%, 10%, 20%, 25%, 30%, 40%, 50%, 60%, 70%, 75%, 80%, 85%, 90%, or 95% of the subject's C17 levels prior to treatment.

In some embodiments, the engineered bacterial cells produce a propionate catabolism enzyme under exogenous environmental conditions, such as the low-oxygen environment of the mammalian gut, to reduce levels of C17 in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, propionylglycine levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of propionylglycine. In some embodiments, the methods may include administration of the compositions of the disclosure to reduce the propionylglycine to undetectable levels in a subject. In some embodiments, the methods may include administration of the compositions of the disclosure to reduce the propionylglycine concentrations to undetectable levels, or to less than about 1%, 2%, 5%, 10%, 20%, 25%, 30%, 40%, 50%, 60%, 70%, 75%, 80%, 85%, 90%, or 95% of the subject's propionylglycine levels prior to treatment.

In some embodiments, the engineered bacterial cells produce a propionate catabolism enzyme under exogenous environmental conditions, such as the low-oxygen environment of the mammalian gut, to reduce levels of propionylglycine in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, lacate levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to reduce levels of lactate. In some embodiments, the methods may include administration of the compositions of the disclosure to reduce the lactate to undetectable levels in a subject. In some embodiments, the methods may include administration of the compositions of the disclosure to reduce the lactate concentrations to undetectable levels, or to less than about 1%, 2%, 5%, 10%, 20%, 25%, 30%, 40%, 50%, 60%, 70%, 75%, 80%, 85%, 90%, or 95% of the subject's lactate levels prior to treatment.

In some embodiments, the engineered bacterial cells produce a propionate catabolism enzyme under exogenous environmental conditions, such as the low-oxygen environment of the mammalian gut, to reduce levels of lactate in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Before, during, and after the administration of the pharmaceutical composition, ratios of C3/C2 and/or C3/C16 and/or C3/C17, and/or C3/Met in the subject may be determined in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the disclosure to alter, e.g., reduce, ratios of C3/C2 and/or C3/C16 and/or C3/C17, and/or C3/Met. In some embodiments, the methods may include administration of the compositions of the disclosure to alter, e.g., reduce, ratios of C3/C2 and/or C3/C16 and/or C3/C17, and/or C3/Met to undetectable levels in a subject. In some embodiments, the methods may include administration of the compositions of the disclosure to alter, e.g., reduce, the ratios of C3/C2 and/or C3/C16 and/or C3/C17, and/or C3/Met to undetectable levels, or to less than about 1%, 2%, 5%, 10%, 20%, 25%, 30%, 40%, 50%, 60%, 70%, 75%, 80%, 85%, 90%, or 95% of the subject's propionate and/or methylmalonate levels prior to treatment.

In some embodiments, the engineered bacterial cells produce a propionate catabolism enzyme under exogenous environmental conditions, such as the low-oxygen environment of the mammalian gut, to alter, e.g., reduce, levels of ratios of C3/C2 and/or C3/C16 and/or C3/C17, and/or C3/Met in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

Certain unmodified bacteria will not have appreciable levels of propionate, propionyl CoA, methylmalonate and/or methylmalonyl CoA processing. In embodiments using genetically modified forms of these bacteria, processing of propionyl CoA and/or methylmalonyl CoA will be appreciable under exogenous environmental conditions.

Propionate levels may be measured by methods known in the art, e.g., blood sampling and mass spectrometry as described in Guenzel et al., 2013, Molecular Ther., 21(7):1316-1323. Methods of measuring methylmalonate are also known in the art (see, e.g., Turgeon et al., Determination of total homocysteine, methylmalonic acid, and 2-methylcitric acid in dried blood spots by tandem mass spectrometry; Clin Chem. 2010 November; 56(11):1686-95; McCann et al., Methylmalonic acid quantification by stable isotope dilution gas chromatography-mass spectrometry from filter paper urine samples, Clin Chem. 1996 June; 42(6 Pt 1):910-4) Carnitines and acylcarnitine levels, including dicarboxylic and hydroxyl acylcarnitines, can be measured according to methods known in the art (see, e.g., Peng et al., Measurement of free carnitine and acylcarnitines in plasma by HILIC-ESI-MS/MS without derivatization J Chromatogr B Analyt Technol Biomed Life Sci. 2013 Aug. 1; 932:12-8).

In some embodiments, propionate catabolism enzyme, e.g., PrpBCDE, expression is measured by methods known in the art. In another embodiment, propionate catabolism enzyme activity is measured by methods known in the art to assess PrpBCDE activity (see propionate catabolism enzyme sections, supra). In another embodiment, propionate catabolism enzyme activity is measured by methods known in the art to assess activity of a PHA pathway circuit described herein. In another embodiment, propionate catabolism enzyme activity is measured by methods known in the art to assess the activity of a MMCA circuit described herein. In another embodiment, propionate catabolism enzyme activity is measured by methods known in the art to assess activity of a MatB circuit described herein, alone or in combination with one or more of PrpBCDE, PHA and or MMCA pathways circuits described herein.

Propionic acid metabolism and/or methylmalonate metabolism, e.g., propionate levels can be analyzed, measured or assessed using C13 propionate. C13 propionate can be administered orally to the subject, e.g., animal or human, and the C13 expired as CO2 can be measured at various intervals, e.g., via Isotope Ratio Mass Spectroscopy). For example, a device for intervallic collection of expired gas from subjects, and subsequent measurement of the isotopic content of such expired gases can be used, (e.g., as described in U.S. Pat. No. 8,293,187 and U.S. Pat. No. 8,721,988 and Chandler and Venditti et al., Long-term rescue of a lethal murine model of methylmalonic acidemia using adeno-associated viral gene therapy. Mol Ther. 2010 January; 18(1):11-6). Such subjects include animals, such as mouse models of PA or MMA described herein or humans. The device includes a constant volume respiratory chamber with provisions to allowing accurate removal of expired gases, and addition of air or other gas to maintain the chamber at a constant volume. The experimental subject (e.g. mammal) is first contacted with a substrate (e.g. amino acid, fatty acid, organic acid) containing an isotope (e.g. 13C) and placed in the chamber. Precisely measured air samples over a time course are collected from the chamber for analysis, while constant air pressure and volume is maintained by the device. The accumulation of the isotope (13C) in the samples over time due to metabolism and the formation of 13CO2 is measured.

In some embodiments, C13 propionate/C13 CO2 measurement method can be used to assess levels of propionate consumption by a genetically engineered bacterial strain in vivo in a subject, e.g., in an animal model of PA and/or MMA or in a human. In a non-limiting example, propionate consumption of a strain comprising gene sequences encoding the MMCA pathway enzymes can be measured. In another non-limiting example, propionate consumption of a strain comprising gene sequences encoding the M2C pathway enzymes can be measured. This method is not suitable for strains which comprise sequences of the Pha pathway, since here the carbon from propionate is deposited as poly-hydroxyalkanoate polymers, rather than exhaled as CO2.

Poly-hydroxyalkanoate polymers can be measured and monitored spectrofluorometrically with Nile red as a fluorochrome (as described in Berlange Herranz et al., Rapid spectrofluorometric screening of poly-hydroxyalkanoate-producing bacteria from microbial mats, the contents of which is herein incorporated by reference in its entirety). For example, in vitro, strains can be grown over night, induced, and moved into 100-ml flasks containing nitrogen-limited MSM, glucose (5 g/1), and 0.5 μg Nile red dye (dissolved in dimethylsulfoxide)/ml. Liquid cultures are then incubated in an orbital shaker (100 rpm) at 30° C., at 1, 2, 4, 6, 12, 24, 48 and 72 h, a 1-ml sample can be removed and then centrifuged in a microcentrifuge at 10,000 rpm at room temperature. According to Berlange Herranz et al., pellets are washed in 1 ml of PBS (pH 7.0), suspended in 1 ml of 0.1 M glycine-HCl (pH 3.0), and incubated at room temperature in the dark for at least 2 h. The relative amount of PHA within the cells, as indicated by the intensity of Nile-red orange fluorescence, can be measured using an appropriate spectrofluorometer. The fluorescence excitation and emission wavelengths of the stained cells in 0.1 M glycine-HCl (pH 3) are 543 nm and 598 nm, respectively. Slits of excitation and emission were set to 10 nm at 900 V.

In certain embodiments, the genetically engineered bacterium is E. coli Nissle. The genetically engineered bacteria may be destroyed, e.g., by defense factors in the gut or blood serum (Sonnenborn et al., 2009), or by activation of a kill switch, several hours or days after administration. Thus, the pharmaceutical composition comprising the engineered bacteria may be re-administered at a therapeutically effective dose and frequency. Length of Nissle residence in vivo in mice can be determined. In alternate embodiments, the genetically engineered bacteria are not destroyed within hours or days after administration and may propagate and colonize the gut.

In one embodiments, the bacterial cells are administered to a subject once daily. In another embodiment, the bacterial cells are administered to a subject twice daily. In another embodiment, the bacterial cells are administered to a subject three times daily. In another embodiment, the bacterial cells are administered to a subject in combination with a meal. In another embodiment, the bacterial cells are administered to a subject prior to a meal. In another embodiment, the bacterial cells are administered to a subject after a meal. The dosage of the pharmaceutical composition and the frequency of administration may be selected based on the severity of the symptoms and the progression of the disease. The appropriate therapeutically effective dose and/or frequency of administration can be selected by a treating clinician.

The methods disclosed herein may comprise administration of a composition alone or in combination with one or more additional therapies, e.g., phenylbutyrate, thiamine supplementation, L-carnitine, and/or a low-protein diet. The pharmaceutical composition may be administered alone or in combination with one or more additional therapeutic agents.

In some embodiments, the composition comprising the genetically engineered bacteria is administered in combination with carnitine. In a non-limiting example, the carnitine is given at a dose of 50-100 mg/kg/day, up to approximately 300 mg/kg/day. IN another example, carnitine is supplements 100 mg/kg/day IV. In some embodiments, the composition comprising the genetically engineered bacteria is administered in combination with propiogenic amino acid-deficient formula and/or protein-free formula. In some embodiments, the composition comprising the genetically engineered bacteria is administered in combination with antioxidants.

In some embodiments, the composition comprising the genetically engineered bacteria is administered in combination with hydroxocobalamin injections. In some embodiments, the hydroxocobalamin injections are 1.0-mg injections every day to every other day. In some embodiments, the composition comprising the genetically engineered bacteria is administered in combination with liver transplantation. In some embodiments, the composition comprising the genetically engineered bacteria is administered in combination with kidney transplantation. In some embodiments, the composition comprising the genetically engineered bacteria is administered in combination with gene therapy. In some embodiments, the gene therapy is AAV-mediated gene therapy. In some embodiments, the gene therapy is intended to replace one or more of enzyme(s) defective in the subject's disorder.

In some embodiments, the composition comprising the genetically engineered bacteria is administered in combination with antibiotics (e.g., neomycin or metronidazole), e.g., if the antibioics do not kill the bacteria.

In some embodiments, the composition comprising the genetically engineered bacteria is administered in combination with N-carbamylglutamate (NCG, Carglumic acid, e.g., 100-250 mg/kg) e.g., if hyperammonemia occurs.

In some embodiments, the composition comprising the genetically engineered bacteria is administered in combination with scavenger medications, e.g., with sodium benzoate (e.g., 250 mg/kg intravenous) or sodium phenylacetate (250 mg/kg), alone or in combination with (Ammunol®), e.g., if hyperammonemia occurs.

In some embodiments, the pharmaceutical composition may be administered in combination with a pharmaceutical composition comprising one or more bacterial strains comprising circuitry for the consumption of ammonium and optionally one or more ammonium transporter(s)/importer(s) and/or arginine exporter(s), as described in co-owned U.S. Pat. No. 9,487,764 and US Patent Publication No. US20160177274, the contents of each of which is herein incorporated by reference in their entireties. Any of the strains described in U.S. Pat. No. 9,487,764 and US Patent Publication No. US20160177274 can be used in the pharmaceutical composition, and are useful for the reduction of ammonia levels in a subject, i.e., for the treatment of hyperammonemia, e.g., as is observed in PA and MMA patients.

In some embodiments, the pharmaceutical composition can be administered with a pharmaceutical composition comprising one or more bacterial strains comprising circuitry for the catabolism of branched chain amino acids (BCAA) (e.g., leucine, isoleucine, and/or valine) and optionally one or more BCAA transporter(s) importer(s) and/or metabolite exporter(s), as described in co-owned International Patent Application No. PCT/US2016/037098, the contents of which is herein incorporated by reference in its entirety. Such strains and pharmaceutical compositions prevent or reduce the production of acetoacetate, acetyl-CoA, propionyl-CoA, and/or propionate from leucine, isoleucine, and/or valine and are therefore useful in the reduction of propionate and/or methylmalonate levels.

In some embodiments three pharmaceutical compositions comprising genetically engineered strains are administered in combination, e.g., a first pharmaceutical composition comprising one or more genetically engineered strains for the catabolism of propionate, described herein, a second pharmaceutical composition comprising one or more strains for the consumption of ammonium, as described in U.S. Pat. No. 9,487,764 and US Patent Publication No. US20160177274, a third pharmaceutical composition comprising one or more strains for the catabolism of branched chain amino acids as described in International Patent Application No. PCT/US2016/037098.

In some embodiments, the composition comprising the genetically engineered bacteria is administered in combination with antiepileptic drugs. In some embodiments, the composition comprising the genetically engineered bacteria is administered in combination with therapies of arrhythmias.

An important consideration in the selection of the one or more additional therapeutic agents is that the agent(s) should be compatible with the bacteria, e.g., the agent(s) must not interfere with or kill the bacteria. In some embodiments, the pharmaceutical composition is administered with food. In alternate embodiments, the pharmaceutical composition is administered before or after eating food. The pharmaceutical composition may be administered in combination with one or more dietary modifications, e.g., low-protein diet and amino acid supplementation. The dosage of the pharmaceutical composition and the frequency of administration may be selected based on the severity of the symptoms and the progression of the disorder. The appropriate therapeutically effective dose and/or frequency of administration can be selected by a treating clinician.

The methods may further comprise isolating a plasma sample from the subject prior to administration of a composition and determining the level of propionate and/or methylmalonate in the sample. In some embodiments, the methods may further comprise isolating a plasma sample from the subject after to administration of a composition and determining the level of the propionate and/or methylmalonate in the sample.

In one embodiment, the methods further comprise comparing the level of the propionate and/or methylmalonate in the plasma sample from the subject after administration of a composition to the subject to the plasma sample from the subject before administration of a composition to the subject. In one embodiment, a reduced level of the propionate and/or methylmalonate in the plasma sample from the subject after administration of a composition indicates that the plasma levels of the propionate and/or methylmalonate are decreased, thereby treating the disorder involving the catabolism of propionate in the subject. In one embodiment, the plasma level of the propionate and/or methylmalonate is decreased at least 10%, 20%, 30%, 40, 50%, 60%, 70%, 80%, 90%, or 100% in the sample after administration of the pharmaceutical composition as compared to the plasma level in the sample before administration of the pharmaceutical composition. In another embodiment, the plasma level of the propionate and/or methylmalonate is decreased at least two-fold, three-fold, four-fold, or five-fold in the sample after administration of the pharmaceutical composition as compared to the plasma level in the sample before administration of the pharmaceutical composition.

In one embodiment, the methods further comprise comparing the level of the propionate and/or methylmalonate in the plasma sample from the subject after administration of a composition to a control level of propionate and/or methylmalonate.

The methods may further comprise isolating a urine sample from the subject prior to administration of a composition and determining the level of propionate and/or methylmalonate in the sample. In some embodiments, the methods may further comprise isolating a urine sample from the subject after to administration of a composition and determining the level of propionate and/or methylmalonate in the sample.

In one embodiment, the methods further comprise comparing the level of the propionate and/or methylmalonate in the urine sample from the subject after administration of a composition to the subject to the urine sample from the subject before administration of a composition to the subject. In one embodiment, a reduced level of the propionate and/or methylmalonate in the urine sample from the subject after administration of a composition indicates that the urine levels of the propionate and/or methylmalonate are decreased, thereby treating the disorder involving the catabolism of propionate in the subject. In one embodiment, the urine level of the propionate and/or methylmalonate is decreased at least 10%, 20%, 30%, 40, 50%, 60%, 70%, 80%, 90%, or 100% in the sample after administration of the pharmaceutical composition as compared to the urine level in the sample before administration of the pharmaceutical composition. In another embodiment, the urine level of propionate and/or methylmalonate is decreased at least two-fold, three-fold, four-fold, or five-fold in the sample after administration of the pharmaceutical composition as compared to the urine level in the sample before administration of the pharmaceutical composition.

In one embodiment, the methods further comprise comparing the level of propionate and/or methylmalonate in the urine sample from the subject after administration of a composition to a control level of propionate and/or methylmalonate.

In some embodiments, reduced levels of 3-hydroxypropionate, 2-methylcitrate, and/or tiglylglycine may be measured. In some embodiments, reduced levels of 3-hydroxypropionate, 2-methylcitrate, and/or tiglylglycine may be detected in the urine upon administration of the pharmaceutical composition.

The methods may further comprise isolating a urine sample from the subject prior to administration of a composition and determining the level of 3-hydroxypropionate, 2-methylcitrate, and/or tiglylglycine in the sample. In some embodiments, the methods may further comprise isolating a urine sample from the subject after to administration of a composition and determining the level of the 3-hydroxypropionate, 2-methylcitrate, and/or tiglylglycine in the sample.

In one embodiment, the methods further comprise comparing the level of the 3-hydroxypropionate, 2-methylcitrate, and/or tiglylglycine in the urine sample from the subject after administration of a composition to the subject to the urine sample from the subject before administration of a composition to the subject. In one embodiment, a reduced level of the 3-hydroxypropionate, 2-methylcitrate, and/or tiglylglycine in the urine sample from the subject after administration of a composition indicates that the urine levels of the 3-hydroxypropionate, 2-methylcitrate, and/or tiglylglycine are decreased, thereby treating the disorder involving the catabolism of propionate in the subject. In one embodiment, the urine level of the 3-hydroxypropionate, 2-methylcitrate, and/or tiglylglycine is decreased at least 10%, 20%, 30%, 40, 50%, 60%, 70%, 80%, 90%, or 100% in the sample after administration of the pharmaceutical composition as compared to the urine level in the sample before administration of the pharmaceutical composition. In another embodiment, the urine level of the 3-hydroxypropionate, 2-methylcitrate, and/or tiglylglycine is decreased at least two-fold, three-fold, four-fold, or five-fold in the sample after administration of the pharmaceutical composition as compared to the urine level in the sample before administration of the pharmaceutical composition.

In some embodiments, plasma concentrations of glycine are measured in a subject. In some embodiments, reduced plasma concentrations of glycine are measured in a subject upon administration of the pharmaceutical composition.

The methods may further comprise isolating a plasma sample from the subject prior to administration of a composition and determining the level of glycine in the sample. In some embodiments, the methods may further comprise isolating a plasma sample from the subject after to administration of a composition and determining the level of the glycine in the sample.

In one embodiment, the methods further comprise comparing the level of the glycine in the plasma sample from the subject after administration of a composition to the subject to the plasma sample from the subject before administration of a composition to the subject. In one embodiment, a reduced level of the glycine in the plasma sample from the subject after administration of a composition indicates that the plasma levels of the glycine are decreased, thereby treating the disorder involving the catabolism of propionate in the subject. In one embodiment, the plasma level of the glycine is decreased at least 10%, 20%, 30%, 40S, 50%, 60%, 70%, 80%, 90%, or 100% in the sample after administration of the pharmaceutical composition as compared to the plasma level in the sample before administration of the pharmaceutical composition. In another embodiment, the plasma level of the glycine is decreased at least two-fold, three-fold, four-fold, or five-fold in the sample after administration of the pharmaceutical composition as compared to the plasma level in the sample before administration of the pharmaceutical composition.

In some embodiments, the levels of C4-dicarboxylic acylcarnitine (C4DC) are measured. In some embodiments, the levels of C4-dicarboxylic acylcarnitine (C4DC) are reduced upon administration of the pharmaceutical composition. In some embodiments, the levels of methylmalonylcarnitine and/or succinylcarnitine are measured. In some embodiments, the levels of methylmalonylcarnitine and/or succinylcarnitine are reduced upon administration of the pharmaceutical composition.

The methods may further comprise isolating a plasma and/or urine sample from the subject prior to administration of a composition and determining the level of C4-dicarboxylic acylcarnitine (C4DC) and/or methylmalonylcarnitine and/or succinylcarnitine in the sample. In some embodiments, the methods may further comprise isolating a plasma and/or urine sample from the subject after to administration of a composition and determining the level of the C4-dicarboxylic acylcarnitine (C4DC) and/or methylmalonylcarnitine and/or succinylcarnitine in the sample.

In one embodiment, the methods further comprise comparing the level of the C4-dicarboxylic acylcarnitine (C4DC) and/or methylmalonylcarnitine and/or succinylcarnitine in the plasma and/or urine sample from the subject after administration of a composition to the subject to the plasma and/or urine sample from the subject before administration of a composition to the subject. In one embodiment, a reduced level of the C4-dicarboxylic acylcarnitine (C4DC) and/or methylmalonylcarnitine and/or succinylcarnitine in the plasma and/or urine sample from the subject after administration of a composition indicates that the plasma and/or urine levels of the C4-dicarboxylic acylcarnitine (C4DC) and/or methylmalonylcarnitine and/or succinylcarnitine are decreased, thereby treating the disorder involving the catabolism of propionate in the subject. In one embodiment, the plasma and/or urine level of the C4-dicarboxylic acylcarnitine (C4DC) and/or methylmalonylcarnitine and/or succinylcarnitine is decreased at least 10%, 20%, 30%, 40S, 50%, 60%, 70%, 80%, 90%, or 100% in the sample after administration of the pharmaceutical composition as compared to the plasma and/or urine level in the sample before administration of the pharmaceutical composition. In another embodiment, the plasma and/or urine level of the C4-dicarboxylic acylcarnitine (C4DC) and/or methylmalonylcarnitine and/or succinylcarnitine is decreased at least two-fold, three-fold, four-fold, or five-fold in the sample after administration of the pharmaceutical composition as compared to the plasma and/or urine level in the sample before administration of the pharmaceutical composition.

In some embodiments, plasma concentrations of propionylcarnitine (C3) are measured. In some embodiments, plasma concentrations of propionylcarnitine (C3) are reduced upon administration of the pharmaceutical composition. In some embodiments, elevated plasma concentrations of propionylcarnitine (C3) are measured relative to acetylcarnitine (C2) (C3/C2 ratio). In some embodiments, the C3/C2 ratio is reduced upon administration of the pharmaceutical composition.

The methods may further comprise isolating a plasma sample from the subject prior to administration of a composition and determining the level of propionylcarnitine in the sample. In some embodiments, the methods may further comprise isolating a plasma sample from the subject after to administration of a composition and determining the level of the propionylcarnitine in the sample.

In one embodiment, the methods further comprise comparing the level of the propionylcarnitine in the plasma sample from the subject after administration of a composition to the subject to the plasma sample from the subject before administration of a composition to the subject. In one embodiment, a reduced level of the propionylcarnitine in the plasma sample from the subject after administration of a composition indicates that the plasma levels of the propionylcarnitine are decreased, thereby treating the disorder involving the catabolism of propionate in the subject. In one embodiment, the plasma level of the propionylcarnitine is decreased at least 10%, 20%, 30%, 40S, 50%, 60%, 70%, 80%, 90%, or 100% in the sample after administration of the pharmaceutical composition as compared to the plasma level in the sample before administration of the pharmaceutical composition. In another embodiment, the plasma level of the propionylcarnitine is decreased at least two-fold, three-fold, four-fold, or five-fold in the sample after administration of the pharmaceutical composition as compared to the plasma level in the sample before administration of the pharmaceutical composition.

In some embodiments, levels of 3-hydroxypalmitoleoyl-carnitine (C16:1 OH) (in plasma and/or urine) are measured. In some embodiments, a reduction in levels of C16:1OH (in plasma and/or urine) are measured upon administration of the pharmaceutical composition. In some embodiments, the ratio of C3/C16 is calculated. In some embodiments, the ratio of C3/C16 is reduced upon administration of the pharmaceutical composition.

The methods may further comprise isolating a plasma and/or urine sample from the subject prior to administration of a composition and determining the level of C16:1OH in the sample. In some embodiments, the methods may further comprise isolating a plasma and/or urine sample from the subject after to administration of a composition and determining the level of the C16:1OH in the sample.

In one embodiment, the methods further comprise comparing the level of the C16:1OH in the plasma and/or urine sample from the subject after administration of a composition to the subject to the plasma and/or urine sample from the subject before administration of a composition to the subject. In one embodiment, a reduced level of the C16:1 OH in the plasma and/or urine sample from the subject after administration of a composition indicates that the plasma and/or urine levels of the C16:1OH are decreased, thereby treating the disorder involving the catabolism of propionate in the subject. In one embodiment, the plasma and/or urine level of the C16:1OH is decreased at least 10%, 20%, 30%, 40, 50%, 60%, 70%, 80%, 90%, or 100% in the sample after administration of the pharmaceutical composition as compared to the plasma and/or urine level in the sample before administration of the pharmaceutical composition. In another embodiment, the plasma and/or urine level of the C16:1OH is decreased at least two-fold, three-fold, four-fold, or five-fold in the sample after administration of the pharmaceutical composition as compared to the plasma and/or urine level in the sample before administration of the pharmaceutical composition.

In some embodiments, levels of heptadecanoylcarnitine (C17) (in plasma and/or urine) are measured. In some embodiments, a reduction in levels of C17 (in plasma and/or urine) are measured upon administration of the pharmaceutical composition. In some embodiments, the ratio of C3/C17 is calculated. In some embodiments, the ratio of C3/C16 is reduced upon administration of the pharmaceutical composition.

The methods may further comprise isolating a plasma and/or urine sample from the subject prior to administration of a composition and determining the level of C17 in the sample. In some embodiments, the methods may further comprise isolating a plasma and/or urine sample from the subject after to administration of a composition and determining the level of the C17 in the sample.

In one embodiment, the methods further comprise comparing the level of the C17 in the plasma and/or urine sample from the subject after administration of a composition to the subject to the plasma and/or urine sample from the subject before administration of a composition to the subject. In one embodiment, a reduced level of the C17 in the plasma and/or urine sample from the subject after administration of a composition indicates that the plasma and/or urine levels of the C17 are decreased, thereby treating the disorder involving the catabolism of propionate in the subject. In one embodiment, the plasma and/or urine level of the C17 is decreased at least 10%, 20%, 30%, 40S, 50%, 60%, 70%, 80%, 90%, or 100% in the sample after administration of the pharmaceutical composition as compared to the plasma and/or urine level in the sample before administration of the pharmaceutical composition. In another embodiment, the plasma and/or urine level of the C17 is decreased at least two-fold, three-fold, four-fold, or five-fold in the sample after administration of the pharmaceutical composition as compared to the plasma and/or urine level in the sample before administration of the pharmaceutical composition.

In one embodiment, the methods further comprise comparing the level of the C17 in the plasma and/or urine sample from the subject after administration of a composition to a control level of propionate and/or methylmalonate.

In some embodiments, levels of propionylglycine (in plasma and/or urine) are measured. In some embodiments, a reduction in levels of propionylglycine (in plasma and/or urine) are measured upon administration of the pharmaceutical composition.

The methods may further comprise isolating a urine sample from the subject prior to administration of a composition and determining the level of propionylglycine in the sample. In some embodiments, the methods may further comprise isolating a urine sample from the subject after to administration of a composition and determining the level of the propionylglycine in the sample.

In one embodiment, the methods further comprise comparing the level of the propionylglycine in the urine sample from the subject after administration of a composition to the subject to the urine sample from the subject before administration of a composition to the subject. In one embodiment, a reduced level of the propionylglycine in the urine sample from the subject after administration of a composition indicates that the urine levels of the propionylglycine are decreased, thereby treating the disorder involving the catabolism of propionate in the subject. In one embodiment, the urine level of the propionylglycine is decreased at least 10%, 20%, 30%, 40S, 50%, 60%, 70%, 80%, 90%, or 100% in the sample after administration of the pharmaceutical composition as compared to the urine level in the sample before administration of the pharmaceutical composition. In another embodiment, the urine level of the propionylglycine is decreased at least two-fold, three-fold, four-fold, or five-fold in the sample after administration of the pharmaceutical composition as compared to the urine level in the sample before administration of the pharmaceutical composition.

In one embodiment, the methods further comprise comparing the level of the propionate and/or methylmalonate in the urine sample from the subject after administration of a composition to a control level of propionate and/or methylmalonate.

In some embodiments, levels of lactate (in urine and/or plasma) are measured. In some embodiments, a reduction in levels of lactate (in urine and/or plasma) are measured upon administration of the pharmaceutical composition.

The methods may further comprise isolating a urine sample from the subject prior to administration of a composition and determining the level of lactate in the sample. In some embodiments, the methods may further comprise isolating a urine sample from the subject after to administration of a composition and determining the level of the lactate in the sample.

In one embodiment, the methods further comprise comparing the level of the lactate in the urine sample from the subject after administration of a composition to the subject to the urine sample from the subject before administration of a composition to the subject. In one embodiment, a reduced level of the lactate in the urine sample from the subject after administration of a composition indicates that the urine levels of the lactate are decreased, thereby treating the disorder involving the catabolism of propionate in the subject. In one embodiment, the urine level of the lactate is decreased at least 10%, 20%, 30%, 40S, 50%, 60%, 70%, 80%, 90%, or 100% in the sample after administration of the pharmaceutical composition as compared to the urine level in the sample before administration of the pharmaceutical composition. In another embodiment, the urine level of the lactate is decreased at least two-fold, three-fold, four-fold, or five-fold in the sample after administration of the pharmaceutical composition as compared to the urine level in the sample before administration of the pharmaceutical composition.

In one embodiment, the methods further comprise comparing the level of the lactate in the urine sample from the subject after administration of a composition to a control level of propionate and/or methylmalonate.

In some embodiments, ratios of C3/C2 and/or C3/C16 and/or C3/C17 and/or C3/Met (in urine and/or plasma) are measured. In some embodiments, a change, e.g., a reduction, in levels of lactate (in urine and/or plasma) are measured upon administration of the pharmaceutical composition.

The methods may further comprise isolating a plasma sample from the subject prior to administration of a composition and determining the ratios of C3/C2 and/or C3/C16 and/or C3/C17 and/or C3/Met in the sample. In some embodiments, the methods may further comprise isolating a plasma sample from the subject after to administration of a composition and determining the level of the ratios of C3/C2 and/or C3/C16 and/or C3/C17 and/or C3/Met in the sample.

In one embodiment, the methods further comprise comparing ratios of C3/C2 and/or C3/C16 and/or C3/C17 and/or C3/Met in the plasma sample from the subject after administration of a composition to the subject to the plasma sample from the subject before administration of a composition to the subject. In one embodiment, reduced ratios of C3/C2 and/or C3/C16 and/or C3/C17 and/or C3/Met in the plasma sample from the subject after administration of a composition indicates that the plasma ratios of C3/C2 and/or C3/C16 and/or C3/C17 and/or C3/Met are decreased, thereby treating the disorder involving the catabolism of propionate in the subject. In one embodiment, the plasma level of the ratios of C3/C2 and/or C3/C16 and/or C3/C17 and/or C3/Met is decreased at least 10%, 20%, 30%, 40, 50%, 60%, 70%, 80%, 90%, or 100% in the sample after administration of the pharmaceutical composition as compared to the plasma level in the sample before administration of the pharmaceutical composition. In another embodiment, the plasma ratios of C3/C2 and/or C3/C16 and/or C3/C17 and/or C3/Met is decreased at least two-fold, three-fold, four-fold, or five-fold in the sample after administration of the pharmaceutical composition as compared to the plasma level in the sample before administration of the pharmaceutical composition.

In one embodiment, the methods further comprise comparing the level of the propionate and/or methylmalonate in the plasma sample from the subject after administration of a composition to control the ratios of C3/C2 and/or C3/C16 and/or C3/C17 and/or C3/Met.

In another embodiment, the methods further comprise comparing the level of methylcitrate, propionylcarnitine, and/or acetylcarnitine, and/or the propionylcarnitine to acetylcarnitine ratio in the plasma sample from the subject after administration of a composition to the subject to the plasma sample from the subject before administration of a composition to the subject. In one embodiment, a reduced level of methylcitrate, propionylcarnitine, and/or acetylcarnitine the propionylcarnitine to acetylcarnitine ratio in the plasma sample from the subject after administration of a composition indicates that the plasma levels of methylcitrate, propionylcarnitine, and/or acetylcarnitine are decreased, thereby treating the disorder involving the catabolism of propionate in the subject. In one embodiment, the plasma level of methylcitrate, propionylcarnitine, and/or acetylcarnitine, and/or the propionylcarnitine to acetylcarnitine ratio is decreased at least 10%, 20%, 30%, 40, 50%, 60%, 70%, 80%, 90%, or 100% in the sample after administration of the pharmaceutical composition as compared to the plasma level in the sample before administration of the pharmaceutical composition. In another embodiment, the plasma level of methylcitrate, propionylcarnitine, and/or acetylcarnitine, and/or the propionylcarnitine to acetylcarnitine ratio is decreased at least two-fold, three-fold, four-fold, or five-fold in the sample after administration of the pharmaceutical composition as compared to the plasma level in the sample before administration of the pharmaceutical composition.

In one embodiment, the methods further comprise comparing the level of methylcitrate, propionylcarnitine, and/or acetylcarnitine, and/or the propionylcarnitine to acetylcarnitine ratio in the plasma sample from the subject after administration of a composition to a control level of methylcitrate, propionylcarnitine, and/or acetylcarnitine.

### Examples

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Development of Engineered Bacterial Cells

### Example 1. Construction of Plasmids Encoding Propionate Catabolism Enzymes and Propionate Transporters (prpBCDE Operon and mtC Gene)

Either the prpBCDE operon from E. coli strain Nissle (SEQ ID NO: 45) or Salmonella (SEQ ID NO: 94) are synthesized (Genewiz), fused to the Tet promoter, cloned into the high-copy plasmid pUC57-Kan by Gibson assembly, and transformed into E. coli DH5α as described herein to generate the plasmid pTet-prpBCDE. The mctC gene of Corynebacterium fused to the Tet promoter (SEQ ID NO: 88) is synthesized (Genewiz) and cloned into the high-copy plasmid pUC57-Kan to generate the plasmid pTet-mctC.

In certain constructs, the prpBCDE operon is operably linked to a FNR-responsive promoter, which may be is further fused to a strong ribosome binding site sequence. For efficient translation, each synthetic gene in the operon was separated by a 15 base pair ribosome binding site derived from the T7 promoter/translational start site. Each gene cassette and regulatory region construct is expressed on a high-copy plasmid, a low-copy plasmid, or a chromosome.

In certain embodiments, the construct is inserted into the bacterial genome at one or more of the following insertion sites in E. coli Nissle: malE/K, araC/BAD, lacZ, thyA, malP/T. Any suitable insertion site may be used (see, e.g., FIG. 32). The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon. At the site of insertion, DNA primers that are homologous to the site of insertion and to the propionate construct are designed. A linear DNA fragment containing the construct with homology to the target site is generated by PCR, and lambda red recombination is performed as described below. The resulting E. coli Nissle bacteria are genetically engineered to express a propionate biosynthesis cassette and produce propionate.

### Example 2. Construction of Plasmids Encoding Propionate Catabolism Enzymes (PHA Pathway)

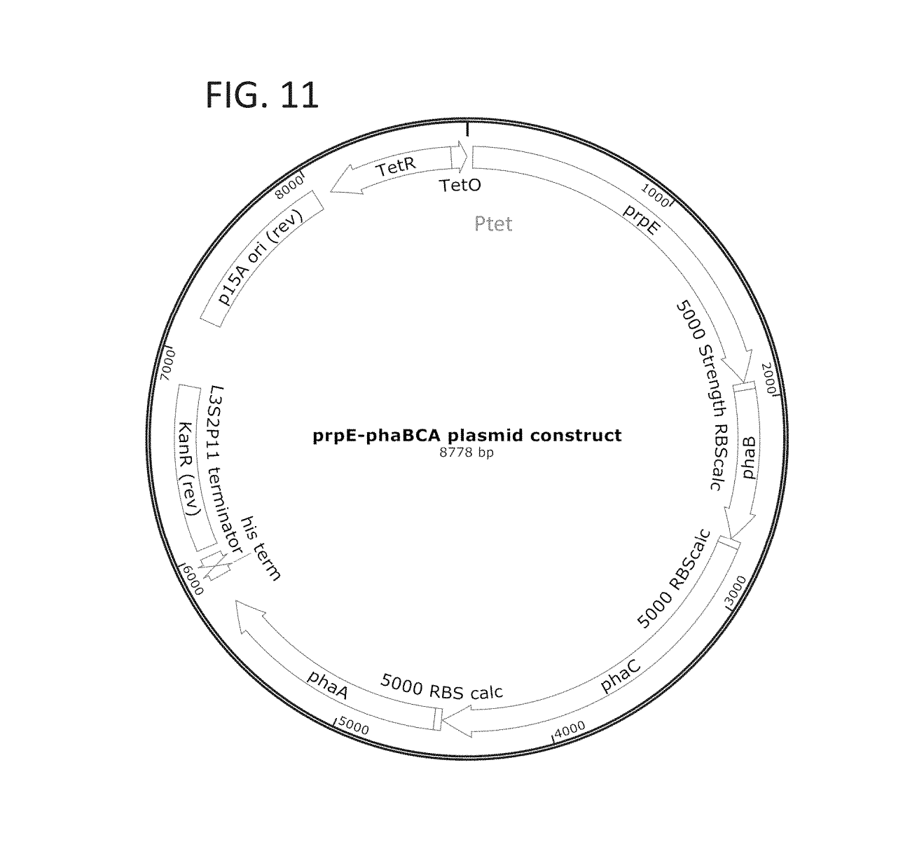

First, the E. coli Nissle prpE gene and phaBCA genes from Acinetobacter sp RA3849 were codon optimized for expression in E. coli Nissle, synthesized, and were placed under the control of an aTc-inducible promoter in a single operon in a high copy plasmid the ˜10-copy plasmid p15A-Kan by Golden Gate assembly, as shown in FIG. 10C and FIG. 11. Corresponding construct sequences are listed in Table 29.

TABLE 29
prpE-PhaBCA pathway circuit sequences

| | | SEQ |
|---|---|---|
| | | ID |
| Description | Sequence | NO |
| Construct | Ttaagacccactttcacatttaagttgtttttctaatccgcatatgatcaattcaaggccgaat | SEQ |
| comprising | aagaaggctggctctgcaccttggtgatcaaataattcgatagcttgtcgtaataatggcgg | ID |
| TetR (reverse | catactatcagtagtaggtgtttccctttcttctttagcgacttgatgctcttgatcttccaatac | NO: 22 |
| orientation, | gcaacctaaagtaaaatgccccacagcgctgagtgcatataatgcattctctagtgaaaaa | |
| italic) and a | ccttgttggcataaaaaggctaattgattttcgagagtttcatactgtttttctgtaggccgtgt | |
| prpE-PhaBCA | acctaaatgtacttttgctccatcgcgatgacttagtaaagcacatctaaaacttttagcgttat | |
| gene cassette | tacgtaaaaaatcttgccagctttccccttctaaagggcaaaagtgagtatggtgcctatcta | |
| driven by a tet | acatctcaatggctaaggcgtcgagcaaagcccgcttattttttacatgccaatacaatgta | |
| promoter | ggctgctctacacctagcttctgggcgagtttacgggttgttaaaccttcgattccgacctca | |
| (italic) (as | ttaagcagctctaatgcgctgttaatcactttacttttatctaatctagacatcatTAATTC | |
| shown in FIG. | CTAATTTTTGTTGACACTCTATCATTGATAGAGTTATTTTAC | |
| 11); ribosome | CACTCCCTATCAGTGATAGAGAAAAGTGAATAAGGCGTAA | |
| binding sites | GTTCAACAGGAGAGCATTATGTCTTTTAGCGAATTTTA | |
| are underlined; | TCAGCGTTCGATTAACGAACCGGAGAAGTTCTGGGCC | |
| L3S2P11 | GAGCAGGCCCGGCGTATTGACTGGCAGACGCCCTTTA | |
| terminator in | CGCAAACGCTCGACCACAGCAACCCGCCGTTTGCCCG | |
| italics and | TTGGTTTTGTGAAGGCCGAACCAACTTGTGTCACAAC | |
| underline; his | GCTATCGACCGCTGGCTGGAGAAACAGCCAGAGGCGC | |
| terminator in | TGGCATTGATTGCCGTCTCTTCGGAAACAGAGGAAGA | |
| bold. | GCGTACCTTTACCTTCCGCCAGTTACATGACGAAGTGA | |
| | ATGCGGTGGCGTCAATGCTGCGCTCACTGGGCGTGCA | |
| | GCGTGGCGATCGGGTGCTGGTGTATATGCCGATGATT | |
| | GCCGAAGCGCATATTACCCTGCTGGCCTGCGCGCGCA | |
| | TTGGTGCTATTCACTCGGTGGTGTTTGGGGGATTTGCT | |
| | TCGCACAGCGTGGCAACGCGAATTGATGACGCTAAAC | |
| | CGGTGCTGATTGTCTCGGCTGATGCCGGGGCGCGCGG | |
| | CGGTAAAATCATTCCGTATAAAAAATTGCTCGACGAT | |
| | GCGATAAGTCAGGCACAGCATCAGCCGCGTCACGTTT | |
| | TACTGGTGGATCGCGGGCTGGCGAAAATGGCGCGCGT | |
| | TAGCGGGCGGGATGTCGATTTCGCGTCGTTGCGCCAT | |
| | CAACACATCGGCGCGCGGGTGCCGGTGGCATGGCTGG | |
| | AATCCAACGAAACCTCCTGCATTCTCTACACCTCCGGC | |
| | ACGACCGGCAAACCTAAAGGTGTGCAGCGTGATGTCG | |
| | GCGGATATGCGGTGGCGCTGGCGACCTCGATGGACAC | |
| | CATTTTTGGCGGCAAAGCGGGCGGCGTGTTCTTTTGTG | |
| | CTTCGGATATCGGCTGGGTGGTAGGGCATTCGTATATC | |
| | GTTTACGCGCCGCTGCTGGCGGGGATGGCGACTATCG | |
| | TTTACGAAGGATTGCCGACCTGGCCGGACTGCGGCGT | |
| | GTGGTGGAAAATTGTCGAGAAATATCAGGTTAGCCGC | |
| | ATGTTCTCAGCGCCGACCGCCATTCGCGTGCTGAAAA | |
| | AATTCCCTACCGCTGAAATTCGCAAACACGATCTTTCG | |
| | TCGCTGGAAGTGCTCTATCTGGCTGGAGAACCGCTGG | |
| | ACGAGCCGACCGCCAGTTGGGTGAGCAATACGCTGGA | |
| | TGTGCCGGTCATCGACAACTACTGGCAGACCGAATCC | |
| | GGCTGGCCGATTATGGCGATTGCTCGCGGTCTGGATG | |
| | ACAGACCGACGCGTCTGGGAAGCCCCGGCGTGCCGAT | |
| | GTATGGCTATAACGTGCAGTTGCTCAATGAAGTCACC | |
| | GGCGAACCGTGTGGCGTCAATGAGAAAGGGATGCTGG | |
| | TAGTGGAGGGGCCATTGCCGCCAGGCTGTATTCAAAC | |
| | CATCTGGGGCGACGACGACCGCTTTGTGAAGACGTAC | |
| | TGGTCGCTGTTTTCCCGTCCGGTGTACGCCACTTTTGA | |
| | CTGGGGCATCCGCGATGCTGACGGTTATCACTTTATTC | |
| | TCGGGCGCACTGACGATGTGATTAACGTTGCCGGACA | |
| | TCGGCTGGGTACGCGTGAGATTGAAGAGAGTATCTCC | |
| | AGTCATCCGGGCGTTGCCGAAGTGGCGGTGGTTGGGG | |
| | TGAAAGATGCGCTGAAAGGGCAGGTGGCGGTGGCGTT | |
| | TGTCATTCCGAAAGAGAGCGACAGTCTGGAAGACCGT | |
| | GAGGTGGCGCACTCGCAAGAGAAGGCGATTATGGCGC | |
| | TGGTGGACAGCCAGATTGGCAACTTTGGCCGCCCGGC | |
| | GCACGTCTGGTTTGTCTCGCAATTGCCAAAAACGCGA | |
| | TCCGGAAAAATGCTGCGCCGCACGATCCAGGCGATTT | |
| | GCGAAGGACGCGATCCTGGGGATCTGACGACCATTGA | |
| | TGATCCGGCGTCGTTGGATCAGATCCGCCAGGCGATG | |
| | GAAGAGTAGTACTGATCAAAAAGGTTAGCCTCAAGAG | |
| | GGTCATAAAAATGTCAGAGCAGAAAGTAGCTCTGGTT | |
| | ACCGGTGCGTTAGGTGGTATCGGAAGTGAGATCTGCC | |
| | GCCAGCTTGTGACCGCCGGGTACAAGATTATCGCCAC | |
| | CGTTGTTCCACGCGAAGAAGACCGCGAAAAACAATGG | |
| | TTGCAAAGTGAGGGGTTTCAAGACTCTGATGTGCGTTT | |
| | CGTATTAACAGATTTAAACAATCACGAAGCTGCGACA | |
| | GCGGCAATTCAAGAAGCGATTGCCGCCGAAGGACGCG | |
| | TTGATGTATTGGTCAACAACGCGGGGATCACGCGCGA | |
| | TGCTACATTTAAGAAAATGTCCTATGAGCAATGGTCC | |
| | CAAGTCATCGACACGAATTTAAAGACTCTTTTTACCGT | |
| | GACCCAGCCAGTATTTAATAAAATGCTTGAACAGAAG | |
| | TCTGGCCGCATCGTAAACATTAGCTCTGTCAATGGTTT | |
| | AAAAGGGCAATTTGGTCAAGCCAACTACTCGGCCTCG | |
| | AAAGCAGGGATTATCGGGTTTACTAAAGCATTGGCGC | |
| | AGGAGGGTGCTCGCTCGAACATTTGCGTCAATGTCGT | |
| | TGCTCCTGGTTACACAGCGACACCCATGGTCACAGCA | |
| | ATGCGCGAGGATGTAATTAAGTCAATCGAAGCTCAAA | |
| | TTCCCCTGCAACGTCTGGCAGCACCGGCGGAGATTGC | |
| | GGCAGCGGTTATGTATTTGGTGAGTGAACACGGTGCA | |
| | TACGTGACGGGCGAAACTTTGAGTATCAACGGCGGGC | |
| | TGTACATGCACTAAAGGTGCTTTTAGTCTAGCGCTAGA | |
| | GCAGGTACCATATTAATGAATCCAAATTCCTTTCAGTT | |
| | TAAAGAGAATATCTTACAGTTTTTCAGCGTGCACGAC | |
| | GATATTTGGAAAAAACTGCAGGAATTTTACTATGGAC | |
| | AATCGCCCATCAATGAAGCGTTGGCGCAGTTAAATAA | |
| | GGAAGACATGAGTTTATTCTTCGAGGCGTTATCAAAA | |
| | AACCCTGCTCGTATGATGGAGATGCAGTGGTCCTGGT | |
| | GGCAAGGGCAGATTCAAATTTACCAGAACGTGTTAAT | |
| | GCGTAGTGTAGCCAAGGACGTAGCCCCCTTTATCCAG | |
| | CCAGAGTCCGGAGATCGTCGCTTCAACTCGCCACTTTG | |
| | GCAAGAACATCCAAATTTTGATTTACTGAGTCAATCCT | |
| | ACTTGTTGTTTTCTCAGTTGGTTCAAAATATGGTGGAT | |
| | GTCGTTGAAGGAGTACCTGATAAGGTCCGCTATCGCA | |
| | TCCATTTCTTTACACGTCAGATGATCAATGCGTTGTCT | |
| | CCTTCTAATTTCCTGTGGACGAACCCTGAAGTAATTCA | |
| | ACAGACGGTCGCTGAACAGGGTGAGAATTTAGTACGC | |
| | GGGATGCAAGTATTTCACGATGATGTAATGAATTCGG | |
| | GTAAATATTTGAGCATCCGTATGGTAAATAGCGACAG | |
| | TTTCTCTCTTGGCAAGGACTTGGCGTATACGCCAGGAG | |
| | CCGTAGTTTTCGAGAACGACATCTTTCAGCTTCTTCAA | |
| | TACGAAGCCACAACCGAGAACGTATATCAAACCCCTA | |
| | TTCTTGTCGTACCTCCCTTCATCAACAAGTACTACGTG | |
| | CTGGACCTGCGCGAACAGAATAGCTTGGTTAATTGGC | |
| | TGCGCCAACAAGGACATACGGTGTTTTTGATGTCGTG | |
| | GCGTAACCCCAACGCAGAGCAGAAGGAGCTTACCTTC | |
| | GCTGACTTAATTACCCAAGGATCGGTAGAAGCATTAC | |
| | GTGTTATCGAAGAAATCACGGGAGAGAAAGAAGCTA | |
| | ACTGTATTGGATATTGCATCGGTGGTACACTTCTGGCT | |
| | GCTACCCAGGCATATTATGTAGCTAAACGCCTGAAAA | |
| | ATCACGTAAAGTCAGCGACTTATATGGCGACGATTAT | |
| | TGATTTTGAGAACCCCGGCTCATTGGGTGTTTTCATTA | |
| | ATGAGCCGGTCGTAAGTGGACTTGAAAACCTTAATAA | |
| | TCAACTTGGTTACTTCGACGGGCGTCAACTTGCAGTGA | |
| | CATTTTCGTTGTTGCGCGAAAACACCTTGTATTGGAAT | |
| | TATTACATCGATAATTACTTGAAGGGTAAGGAACCGT | |
| | CCGACTTTGACATCTTATACTGGAACTCGGATGGTACG | |
| | AATATCCCAGCAAAGATTCACAATTTCCTGTTACGTAA | |
| | CCTTTATCTTAACAACGAACTTATTTCTCCAAATGCCG | |
| | TCAAAGTTAATGGTGTGGGTTTAAACCTTTCGCGCGTG | |
| | AAGACTCCATCATTCTTCATTGCTACGCAGGAGGACC | |
| | ATATCGCATTGTGGGATACCTGTTTTCGCGGCGCGGAT | |
| | TACCTGGGGGGTGAGAGCACACTTGTGCTTGGGGAAA | |
| | GCGGACACGTCGCCGGCATTGTCAACCCGCCTTCTCGT | |
| | AACAAGTATGGTTGTTACACGAACGCCGCCAAGTTTG | |
| | AAAATACCAAGCAATGGCTTGACGGTGCAGAATATCA | |
| | TCCCGAAAGCTGGTGGTTACGTTGGCAGGCATGGGTC | |
| | ACGCCTTATACTGGAGAGCAGGTTCCTGCGCGTAATTT | |
| | GGGAAACGCACAGTACCCCAGTATTGAAGCGGCCCCT | |
| | GGGCGTTATGTGCTGGTAAACCTGTTTTAACGCTCACA | |
| | TACAAGCAATCTATAATTATTCACGGTATAAATGAAA | |
| | GATGTTGTTATCGTAGCCGCTAAACGCACTGCGATCG | |
| | GTTCCTTTCTGGGGAGTCTGGCTTCCCTGAGCGCCCCT | |
| | CAGTTGGGTCAGACGGCTATCCGCGCAGTTTTGGATTC | |
| | TGCAAATGTGAAACCAGAACAAGTGGACCAAGTAATT | |
| | ATGGGGAATGTGCTGACCACCGGCGTTGGGCAAAATC | |
| | CTGCTCGTCAGGCAGCAATCGCCGCTGGGATTCCTGT | |
| | ACAAGTTCCCGCCAGCACGCTTAATGTAGTGTGTGGG | |
| | TCCGGATTACGTGCCGTTCACCTGGCAGCTCAAGCCAT | |
| | CCAATGCGATGAAGCCGATATCGTCGTTGCCGGAGGT | |
| | CAAGAATCAATGTCCCAGTCTGCTCATTACATGCAGCT | |
| | TCGCAATGGCCAGAAAATGGGTAACGCACAGTTAGTC | |
| | GATTCAATGGTGGCCGACGGCTTGACCGACGCGTATA | |
| | ATCAATACCAGATGGGTATCACCGCGGAGAATATCGT | |
| | CGAAAAACTTGGTCTTAATCGTGAAGAACAAGACCAG | |
| | CTTGCTCTGACAAGTCAACAACGTGCTGCAGCAGCGC | |
| | AGGCTGCCGGAAAATTCAAGGATGAAATTGCGGTCGT | |
| | TTCGATTCCCCAGCGCAAAGGAGAGCCGGTCGTCTTC | |
| | GCGGAAGACGAATATATCAAGGCCAATACCTCGTTGG | |
| | AATCCTTGACGAAACTGCGTCCAGCATTCAAAAAAGA | |
| | CGGTTCTGTTACAGCCGGCAACGCATCTGGCATTAAT | |
| | GATGGGGCAGCCGCGGTCCTGATGATGTCCGCCGACA | |
| | AAGCGGCTGAACTGGGCTTAAAGCCTTTAGCACGCAT | |
| | TAAAGGTTACGCGATGTCAGGAATTGAGCCGGAAATC | |
| | ATGGGACTGGGTCCTGTAGACGCCGTTAAGAAAACCC | |
| | TTAATAAGGCTGGTTGGTCCTTAGACCAGGTCGATCTG | |
| | ATCGAGGCCAATGAGGCTTTTGCTGCCCAAGCACTGG | |
| | GAGTAGCCAAGGAGCTTGGGCTGGACCTGGACAAGGT | |
| | AAATGTTAACGGAGGTGCGATCGCGCTGGGACACCCG | |
| | ATCGGGGCTTCGGGTTGTCGTATCTTGGTCACGTTATT | |
| | ACACGAAATGCAGCGTCGTGATGCAAAGAAGGGTATC | |
| | GCCACATTGTGTGTGGGAGGTGGAATGGGGGTGGCGC | |
| | TTGCCGTTGAGCGCGATTAAGGAGGTCGGATAAGGCG | |
| | CTCGCGCCGCATCCGACACCGTGCGCAGATGCCTGAT | |
| | GCGACGCTGACGCGTCTTATCATGCCTCGCTCTCGAGT | |
| | CCCGTCAAGTCAGACGATCGCACGCCCCATGTGAACG | |
| | ATTGGTAAACCCGGTGAACGCATGAGAAAGCCCCCG | |
| | GAAGATCACCTTCCGGGGGCTTTTTTATTGCGCGG | |
| | ACCAAAACGAAAAAAGACGCTCGAAAGCGTCTCTTTTCTG | |
| | GAATTTGGTACCGAGGCGTAATGCTCTGCCAGTGTTAC | |
| | AACCAATTAACCAATTCTGAT | |
| Construct | TAATTCCTAATTTTTGTTGACACTCTATCATTGATAGAGTTA | SEQ |
| comprising a | TTTTACCACTCCCTATCAGTGATAGAGAAAAGTGAATAAG | ID |
| prpE-PhaBCA | GCGTAAGGCGTAAGTTCAACAGGAGAGCATTATGTCT | NO: |
| gene cassette | TTTAGCGAATTTTATCAGCGTTCGATTAACGAACCGGA | 23 |
| under the | GAAGTTCTGGGCCGAGCAGGCCCGGCGTATTGACTGG | |
| control of the | CAGACGCCCTTTACGCAAACGCTCGACCACAGCAACC | |
| Ptet | CGCCGTTTGCCCGTTGGTTTTGTGAAGGCCGAACCAAC | |
| promoter(italic) | TTGTGTCACAACGCTATCGACCGCTGGCTGGAGAAAC | |
| (as shown in | AGCCAGAGGCGCTGGCATTGATTGCCGTCTCTTCGGA | |
| FIG. 11) | AACAGAGGAAGAGCGTACCTTTACCTTCCGCCAGTTA | |
| ribosome | CATGACGAAGTGAATGCGGTGGCGTCAATGCTGCGCT | |
| binding sites | CACTGGGCGTGCAGCGTGGCGATCGGGTGCTGGTGTA | |
| are underlined;. | TATGCCGATGATTGCCGAAGCGCATATTACCCTGCTG | |
| L3S2P11 | GCCTGCGCGCGCATTGGTGCTATTCACTCGGTGGTGTT | |
| terminator in | TGGGGGATTTGCTTCGCACAGCGTGGCAACGCGAATT | |
| italics and | GATGACGCTAAACCGGTGCTGATTGTCTCGGCTGATG | |
| underline; his | CCGGGGCGCGCGGCGGTAAAATCATTCCGTATAAAAA | |
| terminator in | ATTGCTCGACGATGCGATAAGTCAGGCACAGCATCAG | |
| bold | CCGCGTCACGTTTTACTGGTGGATCGCGGGCTGGCGA | |
| | AAATGGCGCGCGTTAGCGGGCGGGATGTCGATTTCGC | |
| | GTCGTTGCGCCATCAACACATCGGCGCGCGGGTGCCG | |
| | GTGGCATGGCTGGAATCCAACGAAACCTCCTGCATTC | |
| | TCTACACCTCCGGCACGACCGGCAAACCTAAAGGTGT | |
| | GCAGCGTGATGTCGGCGGATATGCGGTGGCGCTGGCG | |
| | ACCTCGATGGACACCATTTTTGGCGGCAAAGCGGGCG | |
| | GCGTGTTCTTTTGTGCTTCGGATATCGGCTGGGTGGTA | |
| | GGGCATTCGTATATCGTTTACGCGCCGCTGCTGGCGG | |
| | GGATGGCGACTATCGTTTACGAAGGATTGCCGACCTG | |
| | GCCGGACTGCGGCGTGTGGTGGAAAATTGTCGAGAAA | |
| | TATCAGGTTAGCCGCATGTTCTCAGCGCCGACCGCCAT | |
| | TCGCGTGCTGAAAAAATTCCCTACCGCTGAAATTCGC | |
| | AAACACGATCTTTCGTCGCTGGAAGTGCTCTATCTGGC | |
| | TGGAGAACCGCTGGACGAGCCGACCGCCAGTTGGGTG | |
| | AGCAATACGCTGGATGTGCCGGTCATCGACAACTACT | |
| | GGCAGACCGAATCCGGCTGGCCGATTATGGCGATTGC | |
| | TCGCGGTCTGGATGACAGACCGACGCGTCTGGGAAGC | |
| | CCCGGCGTGCCGATGTATGGCTATAACGTGCAGTTGC | |
| | TCAATGAAGTCACCGGCGAACCGTGTGGCGTCAATGA | |
| | GAAAGGGATGCTGGTAGTGGAGGGGCCATTGCCGCCA | |
| | GGCTGTATTCAAACCATCTGGGGCGACGACGACCGCT | |
| | TTGTGAAGACGTACTGGTCGCTGTTTTCCCGTCCGGTG | |
| | TACGCCACTTTTGACTGGGGCATCCGCGATGCTGACG | |
| | GTTATCACTTTATTCTCGGGCGCACTGACGATGTGATT | |
| | AACGTTGCCGGACATCGGCTGGGTACGCGTGAGATTG | |
| | AAGAGAGTATCTCCAGTCATCCGGGCGTTGCCGAAGT | |
| | GGCGGTGGTTGGGGTGAAAGATGCGCTGAAAGGGCA | |
| | GGTGGCGGTGGCGTTTGTCATTCCGAAAGAGAGCGAC | |
| | AGTCTGGAAGACCGTGAGGTGGCGCACTCGCAAGAGA | |
| | AGGCGATTATGGCGCTGGTGGACAGCCAGATTGGCAA | |
| | CTTTGGCCGCCCGGCGCACGTCTGGTTTGTCTCGCAAT | |
| | TGCCAAAAACGCGATCCGGAAAAATGCTGCGCCGCAC | |
| | GATCCAGGCGATTTGCGAAGGACGCGATCCTGGGGAT | |
| | CTGACGACCATTGATGATCCGGCGTCGTTGGATCAGA | |
| | TCCGCCAGGCGATGGAAGAGTAGTACTGATCAAAAAG | |
| | GTTAGCCTCAAGAGGGTCATAAAAATGTCAGAGCAGA | |
| | AAGTAGCTCTGGTTACCGGTGCGTTAGGTGGTATCGG | |
| | AAGTGAGATCTGCCGCCAGCTTGTGACCGCCGGGTAC | |
| | AAGATTATCGCCACCGTTGTTCCACGCGAAGAAGACC | |
| | GCGAAAAACAATGGTTGCAAAGTGAGGGGTTTCAAGA | |
| | CTCTGATGTGCGTTTCGTATTAACAGATTTAAACAATC | |
| | ACGAAGCTGCGACAGCGGCAATTCAAGAAGCGATTGC | |
| | CGCCGAAGGACGCGTTGATGTATTGGTCAACAACGCG | |
| | GGGATCACGCGCGATGCTACATTTAAGAAAATGTCCT | |
| | ATGAGCAATGGTCCCAAGTCATCGACACGAATTTAAA | |
| | GACTCTTTTTACCGTGACCCAGCCAGTATTTAATAAAA | |
| | TGCTTGAACAGAAGTCTGGCCGCATCGTAAACATTAG | |
| | CTCTGTCAATGGTTTAAAAGGGCAATTTGGTCAAGCC | |
| | AACTACTCGGCCTCGAAAGCAGGGATTATCGGGTTTA | |
| | CTAAAGCATTGGCGCAGGAGGGTGCTCGCTCGAACAT | |
| | TTGCGTCAATGTCGTTGCTCCTGGTTACACAGCGACAC | |
| | CCATGGTCACAGCAATGCGCGAGGATGTAATTAAGTC | |
| | AATCGAAGCTCAAATTCCCCTGCAACGTCTGGCAGCA | |
| | CCGGCGGAGATTGCGGCAGCGGTTATGTATTTGGTGA | |
| | GTGAACACGGTGCATACGTGACGGGCGAAACTTTGAG | |
| | TATCAACGGCGGGCTGTACATGCACTAAAGGTGCTTT | |
| | TAGTCTAGCGCTAGAGCAGGTACCATATTAATGAATC | |
| | CAAATTCCTTTCAGTTTAAAGAGAATATCTTACAGTTT | |
| | TTCAGCGTGCACGACGATATTTGGAAAAAACTGCAGG | |
| | AATTTTACTATGGACAATCGCCCATCAATGAAGCGTT | |
| | GGCGCAGTTAAATAAGGAAGACATGAGTTTATTCTTC | |
| | GAGGCGTTATCAAAAAACCCTGCTCGTATGATGGAGA | |
| | TGCAGTGGTCCTGGTGGCAAGGGCAGATTCAAATTTA | |
| | CCAGAACGTGTTAATGCGTAGTGTAGCCAAGGACGTA | |
| | GCCCCCTTTATCCAGCCAGAGTCCGGAGATCGTCGCTT | |
| | CAACTCGCCACTTTGGCAAGAACATCCAAATTTTGATT | |
| | TACTGAGTCAATCCTACTTGTTGTTTTCTCAGTTGGTTC | |
| | AAAATATGGTGGATGTCGTTGAAGGAGTACCTGATAA | |
| | GGTCCGCTATCGCATCCATTTCTTTACACGTCAGATGA | |
| | TCAATGCGTTGTCTCCTTCTAATTTCCTGTGGACGAAC | |
| | CCTGAAGTAATTCAACAGACGGTCGCTGAACAGGGTG | |
| | AGAATTTAGTACGCGGGATGCAAGTATTTCACGATGA | |
| | TGTAATGAATTCGGGTAAATATTTGAGCATCCGTATG | |
| | GTAAATAGCGACAGTTTCTCTCTTGGCAAGGACTTGG | |
| | CGTATACGCCAGGAGCCGTAGTTTTCGAGAACGACAT | |
| | CTTTCAGCTTCTTCAATACGAAGCCACAACCGAGAAC | |
| | GTATATCAAACCCCTATTCTTGTCGTACCTCCCTTCAT | |
| | CAACAAGTACTACGTGCTGGACCTGCGCGAACAGAAT | |
| | AGCTTGGTTAATTGGCTGCGCCAACAAGGACATACGG | |
| | TGTTTTTGATGTCGTGGCGTAACCCCAACGCAGAGCA | |
| | GAAGGAGCTTACCTTCGCTGACTTAATTACCCAAGGA | |
| | TCGGTAGAAGCATTACGTGTTATCGAAGAAATCACGG | |
| | GAGAGAAAGAAGCTAACTGTATTGGATATTGCATCGG | |
| | TGGTACACTTCTGGCTGCTACCCAGGCATATTATGTAG | |
| | CTAAACGCCTGAAAAATCACGTAAAGTCAGCGACTTA | |
| | TATGGCGACGATTATTGATTTTGAGAACCCCGGCTCAT | |
| | TGGGTGTTTTCATTAATGAGCCGGTCGTAAGTGGACTT | |
| | GAAAACCTTAATAATCAACTTGGTTACTTCGACGGGC | |
| | GTCAACTTGCAGTGACATTTTCGTTGTTGCGCGAAAAC | |
| | ACCTTGTATTGGAATTATTACATCGATAATTACTTGAA | |
| | GGGTAAGGAACCGTCCGACTTTGACATCTTATACTGG | |
| | AACTCGGATGGTACGAATATCCCAGCAAAGATTCACA | |
| | ATTTCCTGTTACGTAACCTTTATCTTAACAACGAACTT | |
| | ATTTCTCCAAATGCCGTCAAAGTTAATGGTGTGGGTTT | |
| | AAACCTTTCGCGCGTGAAGACTCCATCATTCTTCATTG | |
| | CTACGCAGGAGGACCATATCGCATTGTGGGATACCTG | |
| | TTTTCGCGGCGCGGATTACCTGGGGGGTGAGAGCACA | |
| | CTTGTGCTTGGGGAAAGCGGACACGTCGCCGGCATTG | |
| | TCAACCCGCCTTCTCGTAACAAGTATGGTTGTTACACG | |
| | AACGCCGCCAAGTTTGAAAATACCAAGCAATGGCTTG | |
| | ACGGTGCAGAATATCATCCCGAAAGCTGGTGGTTACG | |
| | TTGGCAGGCATGGGTCACGCCTTATACTGGAGAGCAG | |
| | GTTCCTGCGCGTAATTTGGGAAACGCACAGTACCCCA | |
| | GTATTGAAGCGGCCCCTGGGCGTTATGTGCTGGTAAA | |
| | CCTGTTTTAACGCTCACATACAAGCAATCTATAATTAT | |
| | TCACGGTATAAATGAAAGATGTTGTTATCGTAGCCGC | |
| | TAAACGCACTGCGATCGGTTCCTTTCTGGGGAGTCTGG | |
| | CTTCCCTGAGCGCCCCTCAGTTGGGTCAGACGGCTATC | |
| | CGCGCAGTTTTGGATTCTGCAAATGTGAAACCAGAAC | |
| | AAGTGGACCAAGTAATTATGGGGAATGTGCTGACCAC | |
| | CGGCGTTGGGCAAAATCCTGCTCGTCAGGCAGCAATC | |
| | GCCGCTGGGATTCCTGTACAAGTTCCCGCCAGCACGC | |
| | TTAATGTAGTGTGTGGGTCCGGATTACGTGCCGTTCAC | |
| | CTGGCAGCTCAAGCCATCCAATGCGATGAAGCCGATA | |
| | TCGTCGTTGCCGGAGGTCAAGAATCAATGTCCCAGTC | |
| | TGCTCATTACATGCAGCTTCGCAATGGCCAGAAAATG | |
| | GGTAACGCACAGTTAGTCGATTCAATGGTGGCCGACG | |
| | GCTTGACCGACGCGTATAATCAATACCAGATGGGTAT | |
| | CACCGCGGAGAATATCGTCGAAAAACTTGGTCTTAAT | |
| | CGTGAAGAACAAGACCAGCTTGCTCTGACAAGTCAAC | |
| | AACGTGCTGCAGCAGCGCAGGCTGCCGGAAAATTCAA | |
| | GGATGAAATTGCGGTCGTTTCGATTCCCCAGCGCAAA | |
| | GGAGAGCCGGTCGTCTTCGCGGAAGACGAATATATCA | |
| | AGGCCAATACCTCGTTGGAATCCTTGACGAAACTGCG | |
| | TCCAGCATTCAAAAAAGACGGTTCTGTTACAGCCGGC | |
| | AACGCATCTGGCATTAATGATGGGGCAGCCGCGGTCC | |
| | TGATGATGTCCGCCGACAAAGCGGCTGAACTGGGCTT | |
| | AAAGCCTTTAGCACGCATTAAAGGTTACGCGATGTCA | |
| | GGAATTGAGCCGGAAATCATGGGACTGGGTCCTGTAG | |
| | ACGCCGTTAAGAAAACCCTTAATAAGGCTGGTTGGTC | |
| | CTTAGACCAGGTCGATCTGATCGAGGCCAATGAGGCT | |
| | TTTGCTGCCCAAGCACTGGGAGTAGCCAAGGAGCTTG | |
| | GGCTGGACCTGGACAAGGTAAATGTTAACGGAGGTGC | |
| | GATCGCGCTGGGACACCCGATCGGGGCTTCGGGTTGT | |
| | CGTATCTTGGTCACGTTATTACACGAAATGCAGCGTCG | |
| | TGATGCAAAGAAGGGTATCGCCACATTGTGTGTGGGA | |
| | GGTGGAATGGGGGTGGCGCTTGCCGTTGAGCGCGATT | |
| | AAGGAGGTCGGATAAGGCGCTCGCGCCGCATCCGACA | |
| | CCGTGCGCAGATGCCTGATGCGACGCTGACGCGTCTT | |
| | ATCATGCCTCGCTCTCGAGTCCCGTCAAGTCAGACGAT | |
| | CGCACGCCCCATGTGAACGATTGGTAAACCCGGTGAA | |
| | CGCATGAGAAAGCCCCCGGAAGATCACCTTCCGGG | |
| | GGCTTTTTTATTGCGCGGACCAAAACGAAAAAAGACGC | |
| | TCGAAAGCGTCTCTTTTCTGGAATTTGGTACCGAGGCGTA | |
| | ATGCTCTGCCAGTGTTACAACCAATTAACCAATTCTGAT | |
| Construct | TAAGGCGTAAGTTCAACAGGAGAGCATTATGTCTTTT | SEQ |
| comprising a | AGCGAATTTTATCAGCGTTCGATTAACGAACCGGAGA | ID |
| prpE-PhaBCA | AGTTCTGGGCCGAGCAGGCCCGGCGTATTGACTGGCA | NO: |
| gene cassette; | GACGCCCTTTACGCAAACGCTCGACCACAGCAACCCG | 24 |
| (as shown in | CCGTTTGCCCGTTGGTTTTGTGAAGGCCGAACCAACTT | |
| FIG. 11) | GTGTCACAACGCTATCGACCGCTGGCTGGAGAAACAG | |
| ribosome | CCAGAGGCGCTGGCATTGATTGCCGTCTCTTCGGAAA | |
| binding sites | CAGAGGAAGAGCGTACCTTTACCTTCCGCCAGTTACA | |
| are underlined | TGACGAAGTGAATGCGGTGGCGTCAATGCTGCGCTCA | |
| | CTGGGCGTGCAGCGTGGCGATCGGGTGCTGGTGTATA | |
| | TGCCGATGATTGCCGAAGCGCATATTACCCTGCTGGC | |
| | CTGCGCGCGCATTGGTGCTATTCACTCGGTGGTGTTTG | |
| | GGGGATTTGCTTCGCACAGCGTGGCAACGCGAATTGA | |
| | TGACGCTAAACCGGTGCTGATTGTCTCGGCTGATGCC | |
| | GGGGCGCGCGGCGGTAAAATCATTCCGTATAAAAAAT | |
| | TGCTCGACGATGCGATAAGTCAGGCACAGCATCAGCC | |
| | GCGTCACGTTTTACTGGTGGATCGCGGGCTGGCGAAA | |
| | ATGGCGCGCGTTAGCGGGCGGGATGTCGATTTCGCGT | |
| | CGTTGCGCCATCAACACATCGGCGCGCGGGTGCCGGT | |
| | GGCATGGCTGGAATCCAACGAAACCTCCTGCATTCTC | |
| | TACACCTCCGGCACGACCGGCAAACCTAAAGGTGTGC | |
| | AGCGTGATGTCGGCGGATATGCGGTGGCGCTGGCGAC | |
| | CTCGATGGACACCATTTTTGGCGGCAAAGCGGGCGGC | |
| | GTGTTCTTTTGTGCTTCGGATATCGGCTGGGTGGTAGG | |
| | GCATTCGTATATCGTTTACGCGCCGCTGCTGGCGGGG | |
| | ATGGCGACTATCGTTTACGAAGGATTGCCGACCTGGC | |
| | CGGACTGCGGCGTGTGGTGGAAAATTGTCGAGAAATA | |
| | TCAGGTTAGCCGCATGTTCTCAGCGCCGACCGCCATTC | |
| | GCGTGCTGAAAAAATTCCCTACCGCTGAAATTCGCAA | |
| | ACACGATCTTTCGTCGCTGGAAGTGCTCTATCTGGCTG | |
| | GAGAACCGCTGGACGAGCCGACCGCCAGTTGGGTGAG | |
| | CAATACGCTGGATGTGCCGGTCATCGACAACTACTGG | |
| | CAGACCGAATCCGGCTGGCCGATTATGGCGATTGCTC | |
| | GCGGTCTGGATGACAGACCGACGCGTCTGGGAAGCCC | |
| | CGGCGTGCCGATGTATGGCTATAACGTGCAGTTGCTC | |
| | AATGAAGTCACCGGCGAACCGTGTGGCGTCAATGAGA | |
| | AAGGGATGCTGGTAGTGGAGGGGCCATTGCCGCCAGG | |
| | CTGTATTCAAACCATCTGGGGCGACGACGACCGCTTT | |
| | GTGAAGACGTACTGGTCGCTGTTTTCCCGTCCGGTGTA | |
| | CGCCACTTTTGACTGGGGCATCCGCGATGCTGACGGTT | |
| | ATCACTTTATTCTCGGGCGCACTGACGATGTGATTAAC | |
| | GTTGCCGGACATCGGCTGGGTACGCGTGAGATTGAAG | |
| | AGAGTATCTCCAGTCATCCGGGCGTTGCCGAAGTGGC | |
| | GGTGGTTGGGGTGAAAGATGCGCTGAAAGGGCAGGT | |
| | GGCGGTGGCGTTTGTCATTCCGAAAGAGAGCGACAGT | |
| | CTGGAAGACCGTGAGGTGGCGCACTCGCAAGAGAAG | |
| | GCGATTATGGCGCTGGTGGACAGCCAGATTGGCAACT | |
| | TTGGCCGCCCGGCGCACGTCTGGTTTGTCTCGCAATTG | |
| | CCAAAAACGCGATCCGGAAAAATGCTGCGCCGCACGA | |
| | TCCAGGCGATTTGCGAAGGACGCGATCCTGGGGATCT | |
| | GACGACCATTGATGATCCGGCGTCGTTGGATCAGATC | |
| | CGCCAGGCGATGGAAGAGTAGTACTGATCAAAAAGGT | |
| | TAGCCTCAAGAGGGTCATAAAAATGTCAGAGCAGAAA | |
| | GTAGCTCTGGTTACCGGTGCGTTAGGTGGTATCGGAA | |
| | GTGAGATCTGCCGCCAGCTTGTGACCGCCGGGTACAA | |
| | GATTATCGCCACCGTTGTTCCACGCGAAGAAGACCGC | |
| | GAAAAACAATGGTTGCAAAGTGAGGGGTTTCAAGACT | |
| | CTGATGTGCGTTTCGTATTAACAGATTTAAACAATCAC | |
| | GAAGCTGCGACAGCGGCAATTCAAGAAGCGATTGCCG | |
| | CCGAAGGACGCGTTGATGTATTGGTCAACAACGCGGG | |
| | GATCACGCGCGATGCTACATTTAAGAAAATGTCCTAT | |
| | GAGCAATGGTCCCAAGTCATCGACACGAATTTAAAGA | |
| | CTCTTTTTACCGTGACCCAGCCAGTATTTAATAAAATG | |
| | CTTGAACAGAAGTCTGGCCGCATCGTAAACATTAGCT | |
| | CTGTCAATGGTTTAAAAGGGCAATTTGGTCAAGCCAA | |
| | CTACTCGGCCTCGAAAGCAGGGATTATCGGGTTTACT | |
| | AAAGCATTGGCGCAGGAGGGTGCTCGCTCGAACATTT | |
| | GCGTCAATGTCGTTGCTCCTGGTTACACAGCGACACCC | |
| | ATGGTCACAGCAATGCGCGAGGATGTAATTAAGTCAA | |
| | TCGAAGCTCAAATTCCCCTGCAACGTCTGGCAGCACC | |
| | GGCGGAGATTGCGGCAGCGGTTATGTATTTGGTGAGT | |
| | GAACACGGTGCATACGTGACGGGCGAAACTTTGAGTA | |
| | TCAACGGCGGGCTGTACATGCACTAAAGGTGCTTTTA | |
| | GTCTAGCGCTAGAGCAGGTACCATATTAATGAATCCA | |
| | AATTCCTTTCAGTTTAAAGAGAATATCTTACAGTTTTT | |
| | CAGCGTGCACGACGATATTTGGAAAAAACTGCAGGAA | |
| | TTTTACTATGGACAATCGCCCATCAATGAAGCGTTGGC | |
| | GCAGTTAAATAAGGAAGACATGAGTTTATTCTTCGAG | |
| | GCGTTATCAAAAAACCCTGCTCGTATGATGGAGATGC | |
| | AGTGGTCCTGGTGGCAAGGGCAGATTCAAATTTACCA | |
| | GAACGTGTTAATGCGTAGTGTAGCCAAGGACGTAGCC | |
| | CCCTTTATCCAGCCAGAGTCCGGAGATCGTCGCTTCAA | |
| | CTCGCCACTTTGGCAAGAACATCCAAATTTTGATTTAC | |
| | TGAGTCAATCCTACTTGTTGTTTTCTCAGTTGGTTCAA | |
| | AATATGGTGGATGTCGTTGAAGGAGTACCTGATAAGG | |
| | TCCGCTATCGCATCCATTTCTTTACACGTCAGATGATC | |
| | AATGCGTTGTCTCCTTCTAATTTCCTGTGGACGAACCC | |
| | TGAAGTAATTCAACAGACGGTCGCTGAACAGGGTGAG | |
| | AATTTAGTACGCGGGATGCAAGTATTTCACGATGATG | |
| | TAATGAATTCGGGTAAATATTTGAGCATCCGTATGGT | |
| | AAATAGCGACAGTTTCTCTCTTGGCAAGGACTTGGCG | |
| | TATACGCCAGGAGCCGTAGTTTTCGAGAACGACATCT | |
| | TTCAGCTTCTTCAATACGAAGCCACAACCGAGAACGT | |
| | ATATCAAACCCCTATTCTTGTCGTACCTCCCTTCATCA | |
| | ACAAGTACTACGTGCTGGACCTGCGCGAACAGAATAG | |
| | CTTGGTTAATTGGCTGCGCCAACAAGGACATACGGTG | |
| | TTTTTGATGTCGTGGCGTAACCCCAACGCAGAGCAGA | |
| | AGGAGCTTACCTTCGCTGACTTAATTACCCAAGGATC | |
| | GGTAGAAGCATTACGTGTTATCGAAGAAATCACGGGA | |
| | GAGAAAGAAGCTAACTGTATTGGATATTGCATCGGTG | |
| | GTACACTTCTGGCTGCTACCCAGGCATATTATGTAGCT | |
| | AAACGCCTGAAAAATCACGTAAAGTCAGCGACTTATA | |
| | TGGCGACGATTATTGATTTTGAGAACCCCGGCTCATTG | |
| | GGTGTTTTCATTAATGAGCCGGTCGTAAGTGGACTTGA | |
| | AAACCTTAATAATCAACTTGGTTACTTCGACGGGCGTC | |
| | AACTTGCAGTGACATTTTCGTTGTTGCGCGAAAACACC | |
| | TTGTATTGGAATTATTACATCGATAATTACTTGAAGGG | |
| | TAAGGAACCGTCCGACTTTGACATCTTATACTGGAACT | |
| | CGGATGGTACGAATATCCCAGCAAAGATTCACAATTT | |
| | CCTGTTACGTAACCTTTATCTTAACAACGAACTTATTT | |
| | CTCCAAATGCCGTCAAAGTTAATGGTGTGGGTTTAAA | |
| | CCTTTCGCGCGTGAAGACTCCATCATTCTTCATTGCTA | |
| | CGCAGGAGGACCATATCGCATTGTGGGATACCTGTTT | |
| | TCGCGGCGCGGATTACCTGGGGGGTGAGAGCACACTT | |
| | GTGCTTGGGGAAAGCGGACACGTCGCCGGCATTGTCA | |
| | ACCCGCCTTCTCGTAACAAGTATGGTTGTTACACGAAC | |
| | GCCGCCAAGTTTGAAAATACCAAGCAATGGCTTGACG | |
| | GTGCAGAATATCATCCCGAAAGCTGGTGGTTACGTTG | |
| | GCAGGCATGGGTCACGCCTTATACTGGAGAGCAGGTT | |
| | CCTGCGCGTAATTTGGGAAACGCACAGTACCCCAGTA | |
| | TTGAAGCGGCCCCTGGGCGTTATGTGCTGGTAAACCT | |
| | GTTTTAACGCTCACATACAAGCAATCTATAATTATTCA | |
| | CGGTATAAATGAAAGATGTTGTTATCGTAGCCGCTAA | |
| | ACGCACTGCGATCGGTTCCTTTCTGGGGAGTCTGGCTT | |
| | CCCTGAGCGCCCCTCAGTTGGGTCAGACGGCTATCCG | |
| | CGCAGTTTTGGATTCTGCAAATGTGAAACCAGAACAA | |
| | GTGGACCAAGTAATTATGGGGAATGTGCTGACCACCG | |
| | GCGTTGGGCAAAATCCTGCTCGTCAGGCAGCAATCGC | |
| | CGCTGGGATTCCTGTACAAGTTCCCGCCAGCACGCTTA | |
| | ATGTAGTGTGTGGGTCCGGATTACGTGCCGTTCACCTG | |
| | GCAGCTCAAGCCATCCAATGCGATGAAGCCGATATCG | |
| | TCGTTGCCGGAGGTCAAGAATCAATGTCCCAGTCTGC | |
| | TCATTACATGCAGCTTCGCAATGGCCAGAAAATGGGT | |
| | AACGCACAGTTAGTCGATTCAATGGTGGCCGACGGCT | |
| | TGACCGACGCGTATAATCAATACCAGATGGGTATCAC | |
| | CGCGGAGAATATCGTCGAAAAACTTGGTCTTAATCGT | |
| | GAAGAACAAGACCAGCTTGCTCTGACAAGTCAACAAC | |
| | GTGCTGCAGCAGCGCAGGCTGCCGGAAAATTCAAGGA | |
| | TGAAATTGCGGTCGTTTCGATTCCCCAGCGCAAAGGA | |
| | GAGCCGGTCGTCTTCGCGGAAGACGAATATATCAAGG | |
| | CCAATACCTCGTTGGAATCCTTGACGAAACTGCGTCC | |
| | AGCATTCAAAAAAGACGGTTCTGTTACAGCCGGCAAC | |
| | GCATCTGGCATTAATGATGGGGCAGCCGCGGTCCTGA | |
| | TGATGTCCGCCGACAAAGCGGCTGAACTGGGCTTAAA | |
| | GCCTTTAGCACGCATTAAAGGTTACGCGATGTCAGGA | |
| | ATTGAGCCGGAAATCATGGGACTGGGTCCTGTAGACG | |
| | CCGTTAAGAAAACCCTTAATAAGGCTGGTTGGTCCTT | |
| | AGACCAGGTCGATCTGATCGAGGCCAATGAGGCTTTT | |
| | GCTGCCCAAGCACTGGGAGTAGCCAAGGAGCTTGGGC | |
| | TGGACCTGGACAAGGTAAATGTTAACGGAGGTGCGAT | |
| | CGCGCTGGGACACCCGATCGGGGCTTCGGGTTGTCGT | |
| | ATCTTGGTCACGTTATTACACGAAATGCAGCGTCGTG | |
| | ATGCAAAGAAGGGTATCGCCACATTGTGTGTGGGAGG | |
| | TGGAATGGGGGTGGCGCTTGCCGTTGAGCGCGATTAA | |
| prpE sequence | ATGTCTTTTAGCGAATTTTATCAGCGTTCGATTAACGA | SEQ |
| (comprised in | ACCGGAGAAGTTCTGGGCCGAGCAGGCCCGGCGTATT | ID |
| the prpE- | GACTGGCAGACGCCCTTTACGCAAACGCTCGACCACA | NO: |
| PhaBCA | GCAACCCGCCGTTTGCCCGTTGGTTTTGTGAAGGCCGA | 25 |
| construct | ACCAACTTGTGTCACAACGCTATCGACCGCTGGCTGG | |
| shown in FIG. | AGAAACAGCCAGAGGCGCTGGCATTGATTGCCGTCTC | |
| 11) | TTCGGAAACAGAGGAAGAGCGTACCTTTACCTTCCGC | |
| | CAGTTACATGACGAAGTGAATGCGGTGGCGTCAATGC | |
| | TGCGCTCACTGGGCGTGCAGCGTGGCGATCGGGTGCT | |
| | GGTGTATATGCCGATGATTGCCGAAGCGCATATTACC | |
| | CTGCTGGCCTGCGCGCGCATTGGTGCTATTCACTCGGT | |
| | GGTGTTTGGGGGATTTGCTTCGCACAGCGTGGCAACG | |
| | CGAATTGATGACGCTAAACCGGTGCTGATTGTCTCGG | |
| | CTGATGCCGGGGCGCGCGGCGGTAAAATCATTCCGTA | |
| | TAAAAAATTGCTCGACGATGCGATAAGTCAGGCACAG | |
| | CATCAGCCGCGTCACGTTTTACTGGTGGATCGCGGGCT | |
| | GGCGAAAATGGCGCGCGTTAGCGGGCGGGATGTCGAT | |
| | TTCGCGTCGTTGCGCCATCAACACATCGGCGCGCGGG | |
| | TGCCGGTGGCATGGCTGGAATCCAACGAAACCTCCTG | |
| | CATTCTCTACACCTCCGGCACGACCGGCAAACCTAAA | |
| | GGTGTGCAGCGTGATGTCGGCGGATATGCGGTGGCGC | |
| | TGGCGACCTCGATGGACACCATTTTTGGCGGCAAAGC | |
| | GGGCGGCGTGTTCTTTTGTGCTTCGGATATCGGCTGGG | |
| | TGGTAGGGCATTCGTATATCGTTTACGCGCCGCTGCTG | |
| | GCGGGGATGGCGACTATCGTTTACGAAGGATTGCCGA | |
| | CCTGGCCGGACTGCGGCGTGTGGTGGAAAATTGTCGA | |
| | GAAATATCAGGTTAGCCGCATGTTCTCAGCGCCGACC | |
| | GCCATTCGCGTGCTGAAAAAATTCCCTACCGCTGAAA | |
| | TTCGCAAACACGATCTTTCGTCGCTGGAAGTGCTCTAT | |
| | CTGGCTGGAGAACCGCTGGACGAGCCGACCGCCAGTT | |
| | GGGTGAGCAATACGCTGGATGTGCCGGTCATCGACAA | |
| | CTACTGGCAGACCGAATCCGGCTGGCCGATTATGGCG | |
| | ATTGCTCGCGGTCTGGATGACAGACCGACGCGTCTGG | |
| | GAAGCCCCGGCGTGCCGATGTATGGCTATAACGTGCA | |
| | GTTGCTCAATGAAGTCACCGGCGAACCGTGTGGCGTC | |
| | AATGAGAAAGGGATGCTGGTAGTGGAGGGGCCATTGC | |
| | CGCCAGGCTGTATTCAAACCATCTGGGGCGACGACGA | |
| | CCGCTTTGTGAAGACGTACTGGTCGCTGTTTTCCCGTC | |
| | CGGTGTACGCCACTTTTGACTGGGGCATCCGCGATGCT | |
| | GACGGTTATCACTTTATTCTCGGGCGCACTGACGATGT | |
| | GATTAACGTTGCCGGACATCGGCTGGGTACGCGTGAG | |
| | ATTGAAGAGAGTATCTCCAGTCATCCGGGCGTTGCCG | |
| | AAGTGGCGGTGGTTGGGGTGAAAGATGCGCTGAAAG | |
| | GGCAGGTGGCGGTGGCGTTTGTCATTCCGAAAGAGAG | |
| | CGACAGTCTGGAAGACCGTGAGGTGGCGCACTCGCAA | |
| | GAGAAGGCGATTATGGCGCTGGTGGACAGCCAGATTG | |
| | GCAACTTTGGCCGCCCGGCGCACGTCTGGTTTGTCTCG | |
| | CAATTGCCAAAAACGCGATCCGGAAAAATGCTGCGCC | |
| | GCACGATCCAGGCGATTTGCGAAGGACGCGATCCTGG | |
| | GGATCTGACGACCATTGATGATCCGGCGTCGTTGGAT | |
| | CAGATCCGCCAGGCGATGGAAGAGTAG | |
| phaB sequence | ATGTCAGAGCAGAAAGTAGCTCTGGTTACCGGTGCGT | SEQ |
| (comprised in | TAGGTGGTATCGGAAGTGAGATCTGCCGCCAGCTTGT | ID |
| the prpE- | GACCGCCGGGTACAAGATTATCGCCACCGTTGTTCCA | NO: |
| PhaBCA | CGCGAAGAAGACCGCGAAAAACAATGGTTGCAAAGT | 26 |
| construct | GAGGGGTTTCAAGACTCTGATGTGCGTTTCGTATTAAC | |
| shown in FIG. | AGATTTAAACAATCACGAAGCTGCGACAGCGGCAATT | |
| 11) | CAAGAAGCGATTGCCGCCGAAGGACGCGTTGATGTAT | |
| | TGGTCAACAACGCGGGGATCACGCGCGATGCTACATT | |
| | TAAGAAAATGTCCTATGAGCAATGGTCCCAAGTCATC | |
| | GACACGAATTTAAAGACTCTTTTTACCGTGACCCAGCC | |
| | AGTATTTAATAAAATGCTTGAACAGAAGTCTGGCCGC | |
| | ATCGTAAACATTAGCTCTGTCAATGGTTTAAAAGGGC | |
| | AATTTGGTCAAGCCAACTACTCGGCCTCGAAAGCAGG | |
| | GATTATCGGGTTTACTAAAGCATTGGCGCAGGAGGGT | |
| | GCTCGCTCGAACATTTGCGTCAATGTCGTTGCTCCTGG | |
| | TTACACAGCGACACCCATGGTCACAGCAATGCGCGAG | |
| | GATGTAATTAAGTCAATCGAAGCTCAAATTCCCCTGC | |
| | AACGTCTGGCAGCACCGGCGGAGATTGCGGCAGCGGT | |
| | TATGTATTTGGTGAGTGAACACGGTGCATACGTGACG | |
| | GGCGAAACTTTGAGTATCAACGGCGGGCTGTACATGC | |
| | ACTAA | |
| phaC sequence | ATGAATCCAAATTCCTTTCAGTTTAAAGAGAATATCTT | SEQ |
| (comprised in | ACAGTTTTTCAGCGTGCACGACGATATTTGGAAAAAA | ID |
| the prpE- | CTGCAGGAATTTTACTATGGACAATCGCCCATCAATG | NO: |
| PhaBCA | AAGCGTTGGCGCAGTTAAATAAGGAAGACATGAGTTT | 27 |
| construct | ATTCTTCGAGGCGTTATCAAAAAACCCTGCTCGTATGA | |
| shown in FIG. | TGGAGATGCAGTGGTCCTGGTGGCAAGGGCAGATTCA | |
| 11) | AATTTACCAGAACGTGTTAATGCGTAGTGTAGCCAAG | |
| | GACGTAGCCCCCTTTATCCAGCCAGAGTCCGGAGATC | |
| | GTCGCTTCAACTCGCCACTTTGGCAAGAACATCCAAA | |
| | TTTTGATTTACTGAGTCAATCCTACTTGTTGTTTTCTCA | |
| | GTTGGTTCAAAATATGGTGGATGTCGTTGAAGGAGTA | |
| | CCTGATAAGGTCCGCTATCGCATCCATTTCTTTACACG | |
| | TCAGATGATCAATGCGTTGTCTCCTTCTAATTTCCTGT | |
| | GGACGAACCCTGAAGTAATTCAACAGACGGTCGCTGA | |
| | ACAGGGTGAGAATTTAGTACGCGGGATGCAAGTATTT | |
| | CACGATGATGTAATGAATTCGGGTAAATATTTGAGCA | |
| | TCCGTATGGTAAATAGCGACAGTTTCTCTCTTGGCAAG | |
| | GACTTGGCGTATACGCCAGGAGCCGTAGTTTTCGAGA | |
| | ACGACATCTTTCAGCTTCTTCAATACGAAGCCACAACC | |
| | GAGAACGTATATCAAACCCCTATTCTTGTCGTACCTCC | |
| | CTTCATCAACAAGTACTACGTGCTGGACCTGCGCGAA | |
| | CAGAATAGCTTGGTTAATTGGCTGCGCCAACAAGGAC | |
| | ATACGGTGTTTTTGATGTCGTGGCGTAACCCCAACGCA | |
| | GAGCAGAAGGAGCTTACCTTCGCTGACTTAATTACCC | |
| | AAGGATCGGTAGAAGCATTACGTGTTATCGAAGAAAT | |
| | CACGGGAGAGAAAGAAGCTAACTGTATTGGATATTGC | |
| | ATCGGTGGTACACTTCTGGCTGCTACCCAGGCATATTA | |
| | TGTAGCTAAACGCCTGAAAAATCACGTAAAGTCAGCG | |
| | ACTTATATGGCGACGATTATTGATTTTGAGAACCCCGG | |
| | CTCATTGGGTGTTTTCATTAATGAGCCGGTCGTAAGTG | |
| | GACTTGAAAACCTTAATAATCAACTTGGTTACTTCGAC | |
| | GGGCGTCAACTTGCAGTGACATTTTCGTTGTTGCGCGA | |
| | AAACACCTTGTATTGGAATTATTACATCGATAATTACT | |
| | TGAAGGGTAAGGAACCGTCCGACTTTGACATCTTATA | |
| | CTGGAACTCGGATGGTACGAATATCCCAGCAAAGATT | |
| | CACAATTTCCTGTTACGTAACCTTTATCTTAACAACGA | |
| | ACTTATTTCTCCAAATGCCGTCAAAGTTAATGGTGTGG | |
| | GTTTAAACCTTTCGCGCGTGAAGACTCCATCATTCTTC | |
| | ATTGCTACGCAGGAGGACCATATCGCATTGTGGGATA | |
| | CCTGTTTTCGCGGCGCGGATTACCTGGGGGGTGAGAG | |
| | CACACTTGTGCTTGGGGAAAGCGGACACGTCGCCGGC | |
| | ATTGTCAACCCGCCTTCTCGTAACAAGTATGGTTGTTA | |
| | CACGAACGCCGCCAAGTTTGAAAATACCAAGCAATGG | |
| | CTTGACGGTGCAGAATATCATCCCGAAAGCTGGTGGT | |
| | TACGTTGGCAGGCATGGGTCACGCCTTATACTGGAGA | |
| | GCAGGTTCCTGCGCGTAATTTGGGAAACGCACAGTAC | |
| | CCCAGTATTGAAGCGGCCCCTGGGCGTTATGTGCTGG | |
| | TAAACCTGTTTTAA | |
| phaA sequence | ATGAAAGATGTTGTTATCGTAGCCGCTAAACGCACTG | SEQ |
| (comprised in | CGATCGGTTCCTTTCTGGGGAGTCTGGCTTCCCTGAGC | ID |
| the prpE- | GCCCCTCAGTTGGGTCAGACGGCTATCCGCGCAGTTTT | NO: |
| PhaBCA | GGATTCTGCAAATGTGAAACCAGAACAAGTGGACCAA | 28 |
| construct | GTAATTATGGGGAATGTGCTGACCACCGGCGTTGGGC | |
| shown in FIG. | AAAATCCTGCTCGTCAGGCAGCAATCGCCGCTGGGAT | |
| 11) | TCCTGTACAAGTTCCCGCCAGCACGCTTAATGTAGTGT | |
| | GTGGGTCCGGATTACGTGCCGTTCACCTGGCAGCTCA | |
| | AGCCATCCAATGCGATGAAGCCGATATCGTCGTTGCC | |
| | GGAGGTCAAGAATCAATGTCCCAGTCTGCTCATTACA | |
| | TGCAGCTTCGCAATGGCCAGAAAATGGGTAACGCACA | |
| | GTTAGTCGATTCAATGGTGGCCGACGGCTTGACCGAC | |
| | GCGTATAATCAATACCAGATGGGTATCACCGCGGAGA | |
| | ATATCGTCGAAAAACTTGGTCTTAATCGTGAAGAACA | |
| | AGACCAGCTTGCTCTGACAAGTCAACAACGTGCTGCA | |
| | GCAGCGCAGGCTGCCGGAAAATTCAAGGATGAAATTG | |
| | CGGTCGTTTCGATTCCCCAGCGCAAAGGAGAGCCGGT | |
| | CGTCTTCGCGGAAGACGAATATATCAAGGCCAATACC | |
| | TCGTTGGAATCCTTGACGAAACTGCGTCCAGCATTCA | |
| | AAAAAGACGGTTCTGTTACAGCCGGCAACGCATCTGG | |
| | CATTAATGATGGGGCAGCCGCGGTCCTGATGATGTCC | |
| | GCCGACAAAGCGGCTGAACTGGGCTTAAAGCCTTTAG | |
| | CACGCATTAAAGGTTACGCGATGTCAGGAATTGAGCC | |
| | GGAAATCATGGGACTGGGTCCTGTAGACGCCGTTAAG | |
| | AAAACCCTTAATAAGGCTGGTTGGTCCTTAGACCAGG | |
| | TCGATCTGATCGAGGCCAATGAGGCTTTTGCTGCCCA | |
| | AGCACTGGGAGTAGCCAAGGAGCTTGGGCTGGACCTG | |
| | GACAAGGTAAATGTTAACGGAGGTGCGATCGCGCTGG | |
| | GACACCCGATCGGGGCTTCGGGTTGTCGTATCTTGGTC | |
| | ACGTTATTACACGAAATGCAGCGTCGTGATGCAAAGA | |
| | AGGGTATCGCCACATTGTGTGTGGGAGGTGGAATGGG | |
| | GGTGGCGCTTGCCGTTGAGCGCGATTAA | |

The plasmid was transformed into E. coli DH5α as described herein to generate the plasmid pTet-prpE-PhaBCA.

In certain constructs, the prpE-PhaBCA operon is operably linked to a FNR-responsive promoter, which may be is further fused to a strong ribosome binding site sequence. For efficient translation, a 20-30 bp ribosome binding site was included for each synthetic gene in the operon. Each gene cassette and regulatory region construct is expressed on a high-copy plasmid, a low-copy plasmid, or a chromosome.

In certain embodiments, the construct is inserted into the bacterial genome at one or more of the following insertion sites in E. coli Nissle: malE/K, araC/BAD, lacZ, thyA, malP/T. Any suitable insertion site may be used (see, e.g., FIG. 32). The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon. At the site of insertion, DNA primers that are homologous to the site of insertion and to the propionate construct are designed. A linear DNA fragment containing the construct with homology to the target site is generated by PCR, and lambda red recombination is performed as described below. The resulting E. coli Nissle bacteria are genetically engineered to express a propionate biosynthesis cassette and produce propionate.

### Example 3. Construction of Plasmids Encoding Propionate Catabolism Enzymes (MMCA Pathway)

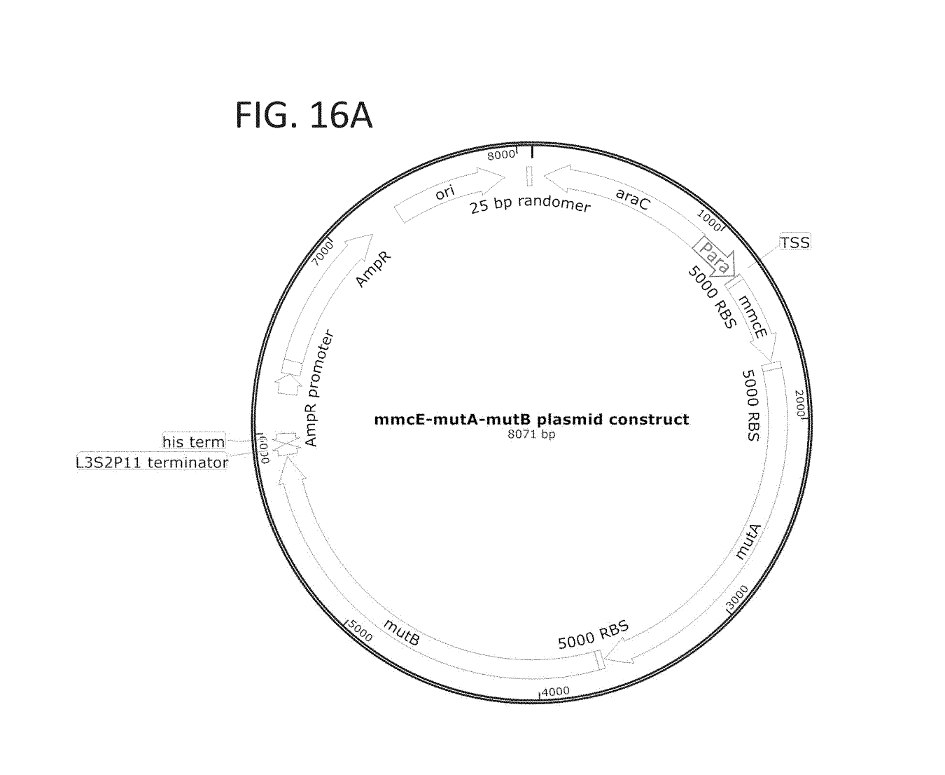

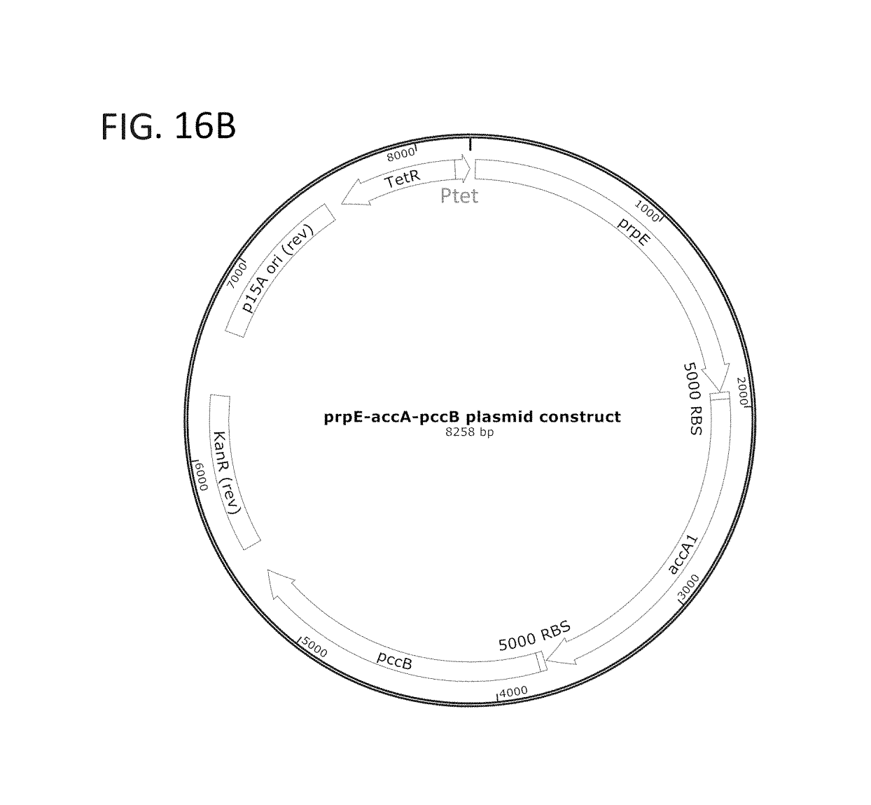

The methylmalonyl-CoA pathway (MMCA) carries out reactions homologous to those in the mammalian pathway. Genes accA (from Streptomyces coelicolor), pccB (from Streptomyces coelicolor), mmcE (from Propionibacterium freudenreichii), and mutAB (from Propionibacterium freudenreichii) were codon-optimized for expression in E. coli Nissle. Two constructs were synthesized, the first with a cassette comprising prpE, pccB, accA1, under the control of an inducible Ptet promoter and the second with a cassette comprising mmcE and mutAB under the control of a second inducible promoter, Para, (as shown in FIG. 15C and FIG. 16A and FIG. 16B).

The constructs were cloned into the plasmids p15a-Kan (pTet-prpE-pccB, -accA1) and an ColE1-Amp (pAra-mmcE-mutAB) by Golden Gate assembly, and transformed into E. coli DH5α as described herein. Sequences of MMCA pathway circuits are listed in Table 30.

TABLE 30
MMCA Pathway Circuit Sequences

| | | SEQ |
|---|---|---|
| Description | Sequence | ID NO |
| Construct comprising AraC | ttattcacaacctgccctaaactcgctcggactcgccccggtgcattttttaaa | SEQ |
| (reverse orientation, lower | tactcgcgagaaatagagttgatcgtcaaaaccgacattgcgaccgacggt | ID NO: |
| case) and a mmcE-mutA- | ggcgataggcatccgggtggtgctcaaaagcagcttcgcctgactgatgc | 29 |
| mutB gene cassette under | gctggtcctcgcgccagcttaatacgctaatccctaactgctggcggaacaa | |
| Para promoter (italics) (as | atgcgacagacgcgacggcgacaggcagacatgctgtgcgacgctggc | |
| shown in FIG. 15B and | gatatcaaaattactgtctgccaggtgatcgctgatgtactgacaagcctcgc | |
| FIG. 16); ribosome | gtacccgattatccatcggtggatggagcgactcgttaatcgcttccatgcg | |
| binding sites are | ccgcagtaacaattgctcaagcagatttatcgccagcaattccgaatagcgc | |
| underlined;. L3S2P11 | ccttccccttgtccggcattaatgatttgcccaaacaggtcgctgaaatgcgg | |
| terminator in italics; his | ctggtgcgcttcatccgggcgaaagaaaccggtattggcaaatatcgacgg | |
| terminator in bold; coding | ccagttaagccattcatgccagtaggcgcgcggacgaaagtaaacccact | |
| regions bold underlined | ggtgataccattcgtgagcctccggatgacgaccgtagtgatgaatctctcc | |
| | aggcgggaacagcaaaatatcacccggtcggcagacaaattctcgtccct | |
| | gatttttcaccaccccctgaccgcgaatggtgagattgagaatataacctttc | |
| | attcccagcggtcggtcgataaaaaaatcgagataaccgttggcctcaatcg | |
| | gcgttaaacccgccaccagatgggcgttaaacgagtatcccggcagcagg | |
| | ggatcattttgcgcttcagccatACTTTTCATACTCCCGCCAT | |
| | TCAGAGAAGAAACCAATTGTCCATATTGCATCAG | |
| | ACATTGCCGTCACTGCGTCTTTTACTGGCTCTTCT | |
| | CGCTAACCCAACCGGTAACCCCGCTTATTAAAAG | |
| | CATTCTGTAACAAAGCGGGACCAAAGCCATGACA | |
| | AAAACGCGTAACAAAAGTGTCTATAATCACGGCA | |
| | GAAAAGTCCACATTGATTATTTGCACGGCGTCAC | |
| | ACTTTGCTATGCCATAGCATTTTTATCCATAAGAT | |
| | TAGCGGATCCAGCCTGACGCTTTTTTTCGCAACT | |
| | CTCTACTGTTTCTCCATACCGGGAAACCACCGC | |
| | GCCCAGCTTAATTTTATGAGTAACGAAGATT | |
| | TATTCATTTGCATCGACCACGTCGCGTATG | |
| | CGTGCCCGGATGCCGATGAAGCTTCTAAGT | |
| | ATTACCAGGAAACATTCGGTTGGCACGAGT | |
| | TGCACCGCGAAGAGAATCCAGAACAGGGC | |
| | GTGGTGGAAATTATGATGGCGCCTGCTGCG | |
| | AAATTGACGGAGCACATGACTCAGGTGCAA | |
| | GTTATGGCGCCTTTGAACGATGAGAGTACG | |
| | GTCGCGAAGTGGCTTGCGAAACACAATGG | |
| | GCGTGCTGGATTGCACCACATGGCATGGCG | |
| | TGTTGATGACATCGACGCAGTGTCCGCAAC | |
| | ACTTCGCGAGCGCGGTGTACAGTTGCTTTA | |
| | CGACGAGCCGAAACTGGGTACAGGTGGGA | |
| | ATCGTATCAACTTCATGCATCCGAAATCTG | |
| | GTAAAGGCGTGCTGATTGAACTGACCCAGT | |
| | ACCCCAAGAATTGATAAAGGTTTTTCCTAAG | |
| | ACGCTAGCGCATAAGGTCCACCAAATGTCAA | |
| | GTACAGACCAAGGCACGAACCCTGCTGACA | |
| | CGGATGATTTAACGCCAACCACATTATCCC | |
| | TGGCTGGTGATTTCCCTAAGGCTACGGAAG | |
| | AGCAGTGGGAGCGCGAGGTTGAAAAGGTG | |
| | TTGAACCGTGGGCGCCCACCCGAGAAGCA | |
| | GTTGACGTTTGCTGAATGTTTAAAACGTCT | |
| | TACTGTGCACACAGTAGATGGCATTGACAT | |
| | CGTTCCAATGTATCGCCCGAAGGATGCCCC | |
| | TAAGAAACTGGGGTATCCAGGGGTTGCTCC | |
| | CTTTACGCGTGGCACTACGGTTCGCAATGG | |
| | GGATATGGACGCTTGGGACGTTCGCGCCCT | |
| | GCACGAAGACCCTGATGAAAAATTCACGCG | |
| | CAAAGCTATTCTGGAGGGGCTGGAGCGCG | |
| | GCGTAACAAGTTTGCTTCTTCGTGTGGACC | |
| | CTGATGCAATCGCTCCCGAACACTTAGACG | |
| | AAGTGTTAAGTGACGTTTTGCTGGAAATGA | |
| | CCAAGGTTGAGGTGTTTTCCCGCTATGATC | |
| | AGGGAGCTGCGGCTGAAGCTCTTGTCTCGG | |
| | TATATGAGCGCAGCGACAAACCGGCTAAAG | |
| | ATTTGGCCTTAAATTTGGGACTGGACCCAA | |
| | TCGCATTTGCTGCACTTCAGGGCACTGAGC | |
| | CAGACTTGACCGTACTTGGTGATTGGGTTC | |
| | GTCGTTTGGCTAAATTCAGCCCAGACTCAC | |
| | GCGCTGTAACAATTGATGCTAATATTTATC | |
| | ACAACGCCGGTGCAGGCGACGTTGCCGAG | |
| | CTGGCCTGGGCACTTGCGACCGGAGCAGA | |
| | GTACGTCCGTGCGCTGGTAGAGCAAGGATT | |
| | CACCGCCACAGAGGCATTTGATACCATTAA | |
| | CTTCCGTGTGACAGCGACCCATGATCAATT | |
| | TTTAACGATTGCCCGCCTTCGTGCGTTACG | |
| | TGAAGCGTGGGCTCGTATCGGTGAGGTATT | |
| | CGGAGTAGATGAGGATAAACGTGGAGCGC | |
| | GCCAGAATGCTATTACGTCCTGGCGTGAAC | |
| | TGACACGCGAGGATCCCTATGTGAACATTT | |
| | TACGTGGAAGTATTGCCACGTTCTCTGCGT | |
| | CCGTTGGGGGCGCGGAGTCTATTACCACTT | |
| | TGCCATTCACGCAGGCATTGGGCCTTCCAG | |
| | AGGATGATTTTCCATTACGTATCGCACGTA | |
| | ATACAGGAATTGTCTTAGCTGAGGAGGTAA | |
| | ACATTGGGCGTGTAAATGACCCTGCCGGGG | |
| | GGTCATACTATGTGGAGAGCTTGACTCGTT | |
| | CTCTTGCAGATGCAGCATGGAAAGAGTTCC | |
| | AAGAGGTTGAAAAGTTGGGTGGTATGTCTA | |
| | AGGCCGTCATGACCGAACACGTCACGAAG | |
| | GTTTTAGATGCTTGCAACGCAGAGCGCGCG | |
| | AAGCGCTTGGCCAACCGCAAGCAACCTATT | |
| | ACGGCAGTTTCCGAATTTCCGATGATTGGC | |
| | GCACGCAGCATTGAGACGAAACCATTTCCG | |
| | GCTGCTCCGGCCCGTAAAGGGCTGGCATG | |
| | GCACCGCGATTCCGAAGTCTTCGAGCAACT | |
| | TATGGACCGCTCCACGTCAGTTTCAGAGCG | |
| | TCCGAAAGTATTTTTAGCATGTCTTGGGAC | |
| | GCGCCGCGATTTTGGAGGACGCGAAGGAT | |
| | TTTCATCTCCGGTTTGGCACATTGCCGGGA | |
| | TTGACACGCCTCAAGTAGAAGGTGGGACGA | |
| | CTGCTGAAATCGTGGAAGCGTTCAAAAAAT | |
| | CTGGGGCCCAAGTCGCCGATTTATGTTCGA | |
| | GTGCCAAAGTGTATGCTCAACAAGGCTTAG | |
| | AGGTGGCAAAGGCTCTGAAAGCGGCTGGG | |
| | GCTAAGGCGCTGTATTTGAGCGGAGCATTT | |
| | AAGGAGTTCGGAGACGATGCAGCGGAAGC | |
| | CGAAAAACTTATCGACGGACGCCTTTTCAT | |
| | GGGCATGGATGTCGTTGACACCCTGTCTTC | |
| | CACTTTAGATATCCTTGGAGTGGCGAAGTG | |
| | ATAAGCTTAAAACAATTTACATCCGGCCGGAA | |
| | CTTACTATGTCTACCTTACCTCGCTTTGACA | |
| | GTGTTGATTTAGGAAATGCGCCGGTCCCAG | |
| | CAGATGCTGCACGTCGTTTTGAGGAACTTG | |
| | CGGCGAAAGCCGGGACCGGCGAAGCCTGG | |
| | GAAACTGCGGAACAAATTCCAGTAGGCACG | |
| | TTGTTTAATGAAGACGTATACAAGGACATG | |
| | GATTGGCTTGATACTTACGCTGGCATTCCT | |
| | CCCTTCGTCCATGGTCCGTACGCTACTATG | |
| | TATGCATTTCGTCCTTGGACCATTCGCCAA | |
| | TATGCCGGTTTTTCGACTGCAAAGGAGTCA | |
| | AACGCATTTTACCGTCGTAATTTGGCTGCA | |
| | GGCCAGAAAGGTCTTAGTGTTGCTTTTGAC | |
| | TTACCCACTCACCGCGGTTATGATTCCGAC | |
| | AACCCCCGCGTGGCCGGAGATGTTGGTATG | |
| | GCCGGTGTGGCTATCGATTCGATTTATGAC | |
| | ATGCGTGAGCTGTTCGCCGGCATCCCATTA | |
| | GATCAGATGAGCGTGTCGATGACAATGAAC | |
| | GGTGCTGTCTTGCCGATTTTGGCTCTTTAT | |
| | GTGGTTACGGCGGAGGAGCAAGGCGTGAA | |
| | GCCAGAACAACTGGCGGGTACTATTCAAAA | |
| | TGATATTCTGAAGGAATTTATGGTTCGTAA | |
| | TACATATATTTACCCGCCGCAACCTAGTAT | |
| | GCGCATTATCAGCGAGATTTTTGCATACAC | |
| | ATCAGCAAACATGCCGAAGTGGAACTCCAT | |
| | TAGTATCAGCGGCTATCATATGCAGGAGGC | |
| | TGGAGCGACTGCGGATATCGAGATGGCGT | |
| | ATACCTTAGCTGATGGAGTTGATTACATCC | |
| | GTGCTGGTGAGTCAGTAGGACTTAATGTGG | |
| | ACCAATTTGCTCCACGCCTGTCCTTCTTCT | |
| | GGGGCATTGGTATGAACTTTTTCATGGAGG | |
| | TAGCGAAGTTACGCGCTGCCCGTATGCTGT | |
| | GGGCGAAGCTTGTCCACCAGTTCGGCCCGA | |
| | AAAACCCGAAGAGTATGTCTCTGCGCACGC | |
| | ACTCTCAAACATCGGGTTGGTCTTTGACAG | |
| | CTCAAGACGTATATAATAACGTTGTACGTA | |
| | CATGCATCGAAGCCATGGCTGCTACTCAAG | |
| | GCCATACTCAATCACTTCATACAAATTCGTT | |
| | GGATGAAGCCATTGCATTGCCTACGGACTT | |
| | TTCAGCCCGCATTGCCCGCAATACTCAATT | |
| | ATTTCTGCAACAAGAGAGCGGGACGACTCG | |
| | TGTGATCGACCCTTGGTCAGGTTCCGCATA | |
| | CGTCGAAGAGTTGACTTGGGATTTAGCTCG | |
| | TAAAGCCTGGGGGCATATTCAGGAGGTTGA | |
| | GAAGGTGGGGGGCATGGCTAAGGCAATCG | |
| | AGAAGGGGATTCCGAAGATGCGCATTGAG | |
| | GAGGCAGCCGCCCGTACCCAAGCACGTATT | |
| | GATTCGGGACGCCAGCCATTAATTGGGGTC | |
| | AATAAATACCGTCTGGAGCACGAACCACCC | |
| | CTGGATGTGTTGAAGGTAGACAATAGCACC | |
| | GTGTTAGCTGAGCAAAAGGCCAAACTTGTT | |
| | AAATTGCGCGCAGAACGCGACCCAGAAAA | |
| | GGTCAAGGCTGCTCTGGACAAAATCACTTG | |
| | GGCGGCTGGCAATCCTGATGATAAAGACCC | |
| | TGATCGCAACTTATTAAAGCTGTGCATTGA | |
| | TGCGGGGCGCGCGATGGCAACGGTAGGAG | |
| | AGATGAGTGACGCTTTAGAGAAAGTTTTTG | |
| | GGCGCTACACAGCGCAAATTCGCACTATTT | |
| | CAGGAGTATATTCAAAAGAAGTCAAAAACA | |
| | CTCCGGAAGTCGAGGAGGCTCGCGAACTG | |
| | GTAGAAGAGTTTGAGCAGGCCGAAGGCCG | |
| | TCGCCCACGTATCCTGCTGGCTAAAATGGG | |
| | GCAGGACGGTCATGACCGTGGGCAAAAGG | |
| | TCATCGCGACTGCATACGCCGATTTGGGAT | |
| | TTGACGTGGACGTTGGCCCGTTATTCCAAA | |
| | CTCCCGAGGAAACTGCTCGCCAAGCCGTCG | |
| | AAGCCGATGTGCACGTAGTGGGGGTGAGC | |
| | TCTCTGGCGGGAGGGCATCTTACGCTTGTG | |
| | CCTGCGCTTCGCAAAGAGCTGGACAAGTTG | |
| | GGTCGTCCAGATATTCTGATTACCGTAGGA | |
| | GGGGTTATTCCCGAGCAGGACTTCGATGAG | |
| | CTTCGTAAGGATGGCGCTGTTGAAATCTAC | |
| | ACACCGGGGACGGTCATTCCAGAATCGGCT | |
| | ATCTCTTTAGTTAAAAAATTGCGCGCCTCC | |
| | CTGGATGCTTGATAAGGAGCTCGGTACCAAAT | |
| | TCCAGAAAAGAGACGCTTTCGAGCGTCTTTTTTC | |
| | GTTTTGGTCCGCGCAATAAAAAAGCCCCCGG | |
| | AAGGTGATCTTCCGGGGGCTTTCTCATGCG | |
| | TT | |
| Construct comprising a | ACTTTTCATACTCCCGCCATTCAGAGAAGAAACC | SEQ |
| mmcE-mutA-mutB gene | AATTGTCCATATTGCATCAGACATTGCCGTCACTG | ID NO: |
| cassette under the control | CGTCTTTTACTGGCTCTTCTCGCTAACCCAACCG | 30 |
| of the Para promoter (as | GTAACCCCGCTTATTAAAAGCATTCTGTAACAAAG | |
| shown in FIG. 15B and | CGGGACCAAAGCCATGACAAAAACGCGTAACAAA | |
| FIG. 16) ribosome binding | AGTGTCTATAATCACGGCAGAAAAGTCCACATTG | |
| sites are underlined;. | ATTATTTGCACGGCGTCACACTTTGCTATGCCATA | |
| coding regions bold | GCATTTTTATCCATAAGATTAGCGGATCCAGCCT | |
| underlined | GACGCTTTTTTTCGCAACTCTCTACTGTTTCTCCA | |
| | TACCGGGAAACCACCGCGCCCAGCTTAATTTT | |
| | ATGAGTAACGAAGATTTATTCATTTGCATC | |
| | GACCACGTCGCGTATGCGTGCCCGGATGCC | |
| | GATGAAGCTTCTAAGTATTACCAGGAAACA | |
| | TTCGGTTGGCACGAGTTGCACCGCGAAGAG | |
| | AATCCAGAACAGGGCGTGGTGGAAATTATG | |
| | ATGGCGCCTGCTGCGAAATTGACGGAGCAC | |
| | ATGACTCAGGTGCAAGTTATGGCGCCTTTG | |
| | AACGATGAGAGTACGGTCGCGAAGTGGCTT | |
| | GCGAAACACAATGGGCGTGCTGGATTGCAC | |
| | CACATGGCATGGCGTGTTGATGACATCGAC | |
| | GCAGTGTCCGCAACACTTCGCGAGCGCGGT | |
| | GTACAGTTGCTTTACGACGAGCCGAAACTG | |
| | GGTACAGGTGGGAATCGTATCAACTTCATG | |
| | CATCCGAAATCTGGTAAAGGCGTGCTGATT | |
| | GAACTGACCCAGTACCCCAAGAATTGATAA | |
| | AGGTTTTTCCTAAGACGCTAGCGCATAAGGTC | |
| | CACCAAATGTCAAGTACAGACCAAGGCACG | |
| | AACCCTGCTGACACGGATGATTTAACGCCA | |
| | ACCACATTATCCCTGGCTGGTGATTTCCCT | |
| | AAGGCTACGGAAGAGCAGTGGGAGCGCGA | |
| | GGTTGAAAAGGTGTTGAACCGTGGGCGCC | |
| | CACCCGAGAAGCAGTTGACGTTTGCTGAAT | |
| | GTTTAAAACGTCTTACTGTGCACACAGTAG | |
| | ATGGCATTGACATCGTTCCAATGTATCGCC | |
| | CGAAGGATGCCCCTAAGAAACTGGGGTATC | |
| | CAGGGGTTGCTCCCTTTACGCGTGGCACTA | |
| | CGGTTCGCAATGGGGATATGGACGCTTGG | |
| | GACGTTCGCGCCCTGCACGAAGACCCTGAT | |
| | GAAAAATTCACGCGCAAAGCTATTCTGGAG | |
| | GGGCTGGAGCGCGGCGTAACAAGTTTGCTT | |
| | CTTCGTGTGGACCCTGATGCAATCGCTCCC | |
| | GAACACTTAGACGAAGTGTTAAGTGACGTT | |
| | TTGCTGGAAATGACCAAGGTTGAGGTGTTT | |
| | TCCCGCTATGATCAGGGAGCTGCGGCTGAA | |
| | GCTCTTGTCTCGGTATATGAGCGCAGCGAC | |
| | AAACCGGCTAAAGATTTGGCCTTAAATTTG | |
| | GGACTGGACCCAATCGCATTTGCTGCACTT | |
| | CAGGGCACTGAGCCAGACTTGACCGTACTT | |
| | GGTGATTGGGTTCGTCGTTTGGCTAAATTC | |
| | AGCCCAGACTCACGCGCTGTAACAATTGAT | |
| | GCTAATATTTATCACAACGCCGGTGCAGGC | |
| | GACGTTGCCGAGCTGGCCTGGGCACTTGC | |
| | GACCGGAGCAGAGTACGTCCGTGCGCTGG | |
| | TAGAGCAAGGATTCACCGCCACAGAGGCAT | |
| | TTGATACCATTAACTTCCGTGTGACAGCGA | |
| | CCCATGATCAATTTTTAACGATTGCCCGCC | |
| | TTCGTGCGTTACGTGAAGCGTGGGCTCGTA | |
| | TCGGTGAGGTATTCGGAGTAGATGAGGATA | |
| | AACGTGGAGCGCGCCAGAATGCTATTACGT | |
| | CCTGGCGTGAACTGACACGCGAGGATCCCT | |
| | ATGTGAACATTTTACGTGGAAGTATTGCCA | |
| | CGTTCTCTGCGTCCGTTGGGGGCGCGGAGT | |
| | CTATTACCACTTTGCCATTCACGCAGGCAT | |
| | TGGGCCTTCCAGAGGATGATTTTCCATTAC | |
| | GTATCGCACGTAATACAGGAATTGTCTTAG | |
| | CTGAGGAGGTAAACATTGGGCGTGTAAATG | |
| | ACCCTGCCGGGGGGTCATACTATGTGGAGA | |
| | GCTTGACTCGTTCTCTTGCAGATGCAGCAT | |
| | GGAAAGAGTTCCAAGAGGTTGAAAAGTTGG | |
| | GTGGTATGTCTAAGGCCGTCATGACCGAAC | |
| | ACGTCACGAAGGTTTTAGATGCTTGCAACG | |
| | CAGAGCGCGCGAAGCGCTTGGCCAACCGC | |
| | AAGCAACCTATTACGGCAGTTTCCGAATTT | |
| | CCGATGATTGGCGCACGCAGCATTGAGACG | |
| | AAACCATTTCCGGCTGCTCCGGCCCGTAAA | |
| | GGGCTGGCATGGCACCGCGATTCCGAAGT | |
| | CTTCGAGCAACTTATGGACCGCTCCACGTC | |
| | AGTTTCAGAGCGTCCGAAAGTATTTTTAGC | |
| | ATGTCTTGGGACGCGCCGCGATTTTGGAGG | |
| | ACGCGAAGGATTTTCATCTCCGGTTTGGCA | |
| | CATTGCCGGGATTGACACGCCTCAAGTAGA | |
| | AGGTGGGACGACTGCTGAAATCGTGGAAG | |
| | CGTTCAAAAAATCTGGGGCCCAAGTCGCCG | |
| | ATTTATGTTCGAGTGCCAAAGTGTATGCTC | |
| | AACAAGGCTTAGAGGTGGCAAAGGCTCTGA | |
| | AAGCGGCTGGGGCTAAGGCGCTGTATTTGA | |
| | GCGGAGCATTTAAGGAGTTCGGAGACGAT | |
| | GCAGCGGAAGCCGAAAAACTTATCGACGG | |
| | ACGCCTTTTCATGGGCATGGATGTCGTTGA | |
| | CACCCTGTCTTCCACTTTAGATATCCTTGG | |
| | AGTGGCGAAGTGATAAGCTTAAAACAATTTA | |
| | CATCCGGCCGGAACTTACTATGTCTACCTTA | |
| | CCTCGCTTTGACAGTGTTGATTTAGGAAAT | |
| | GCGCCGGTCCCAGCAGATGCTGCACGTCGT | |
| | TTTGAGGAACTTGCGGCGAAAGCCGGGAC | |
| | CGGCGAAGCCTGGGAAACTGCGGAACAAA | |
| | TTCCAGTAGGCACGTTGTTTAATGAAGACG | |
| | TATACAAGGACATGGATTGGCTTGATACTT | |
| | ACGCTGGCATTCCTCCCTTCGTCCATGGTC | |
| | CGTACGCTACTATGTATGCATTTCGTCCTT | |
| | GGACCATTCGCCAATATGCCGGTTTTTCGA | |
| | CTGCAAAGGAGTCAAACGCATTTTACCGTC | |
| | GTAATTTGGCTGCAGGCCAGAAAGGTCTTA | |
| | GTGTTGCTTTTGACTTACCCACTCACCGCG | |
| | GTTATGATTCCGACAACCCCCGCGTGGCCG | |
| | GAGATGTTGGTATGGCCGGTGTGGCTATCG | |
| | ATTCGATTTATGACATGCGTGAGCTGTTCG | |
| | CCGGCATCCCATTAGATCAGATGAGCGTGT | |
| | CGATGACAATGAACGGTGCTGTCTTGCCGA | |
| | TTTTGGCTCTTTATGTGGTTACGGCGGAGG | |
| | AGCAAGGCGTGAAGCCAGAACAACTGGCG | |
| | GGTACTATTCAAAATGATATTCTGAAGGAA | |
| | TTTATGGTTCGTAATACATATATTTACCCGC | |
| | CGCAACCTAGTATGCGCATTATCAGCGAGA | |
| | TTTTTGCATACACATCAGCAAACATGCCGA | |
| | AGTGGAACTCCATTAGTATCAGCGGCTATC | |
| | ATATGCAGGAGGCTGGAGCGACTGCGGAT | |
| | ATCGAGATGGCGTATACCTTAGCTGATGGA | |
| | GTTGATTACATCCGTGCTGGTGAGTCAGTA | |
| | GGACTTAATGTGGACCAATTTGCTCCACGC | |
| | CTGTCCTTCTTCTGGGGCATTGGTATGAAC | |
| | TTTTTCATGGAGGTAGCGAAGTTACGCGCT | |
| | GCCCGTATGCTGTGGGCGAAGCTTGTCCAC | |
| | CAGTTCGGCCCGAAAAACCCGAAGAGTATG | |
| | TCTCTGCGCACGCACTCTCAAACATCGGGT | |
| | TGGTCTTTGACAGCTCAAGACGTATATAAT | |
| | AACGTTGTACGTACATGCATCGAAGCCATG | |
| | GCTGCTACTCAAGGCCATACTCAATCACTT | |
| | CATACAAATTCGTTGGATGAAGCCATTGCA | |
| | TTGCCTACGGACTTTTCAGCCCGCATTGCC | |
| | CGCAATACTCAATTATTTCTGCAACAAGAG | |
| | AGCGGGACGACTCGTGTGATCGACCCTTGG | |
| | TCAGGTTCCGCATACGTCGAAGAGTTGACT | |
| | TGGGATTTAGCTCGTAAAGCCTGGGGGCAT | |
| | ATTCAGGAGGTTGAGAAGGTGGGGGGCAT | |
| | GGCTAAGGCAATCGAGAAGGGGATTCCGA | |
| | AGATGCGCATTGAGGAGGCAGCCGCCCGT | |
| | ACCCAAGCACGTATTGATTCGGGACGCCAG | |
| | CCATTAATTGGGGTCAATAAATACCGTCTG | |
| | GAGCACGAACCACCCCTGGATGTGTTGAAG | |
| | GTAGACAATAGCACCGTGTTAGCTGAGCAA | |
| | AAGGCCAAACTTGTTAAATTGCGCGCAGAA | |
| | CGCGACCCAGAAAAGGTCAAGGCTGCTCTG | |
| | GACAAAATCACTTGGGCGGCTGGCAATCCT | |
| | GATGATAAAGACCCTGATCGCAACTTATTA | |
| | AAGCTGTGCATTGATGCGGGGCGCGCGAT | |
| | GGCAACGGTAGGAGAGATGAGTGACGCTT | |
| | TAGAGAAAGTTTTTGGGCGCTACACAGCGC | |
| | AAATTCGCACTATTTCAGGAGTATATTCAA | |
| | AAGAAGTCAAAAACACTCCGGAAGTCGAGG | |
| | AGGCTCGCGAACTGGTAGAAGAGTTTGAGC | |
| | AGGCCGAAGGCCGTCGCCCACGTATCCTGC | |
| | TGGCTAAAATGGGGCAGGACGGTCATGAC | |
| | CGTGGGCAAAAGGTCATCGCGACTGCATAC | |
| | GCCGATTTGGGATTTGACGTGGACGTTGGC | |
| | CCGTTATTCCAAACTCCCGAGGAAACTGCT | |
| | CGCCAAGCCGTCGAAGCCGATGTGCACGTA | |
| | GTGGGGGTGAGCTCTCTGGCGGGAGGGCA | |
| | TCTTACGCTTGTGCCTGCGCTTCGCAAAGA | |
| | GCTGGACAAGTTGGGTCGTCCAGATATTCT | |
| | GATTACCGTAGGAGGGGTTATTCCCGAGCA | |
| | GGACTTCGATGAGCTTCGTAAGGATGGCGC | |
| | TGTTGAAATCTACACACCGGGGACGGTCAT | |
| | TCCAGAATCGGCTATCTCTTTAGTTAAAAA | |
| | ATTGCGCGCCTCCCTGGATGCT | |
| Construct comprising a | GGGAAACCACCGCGCCCAGCTTAATTTTATGA | SEQ |
| mmcE-mutA-mutB gene | GTAACGAAGATTTATTCATTTGCATCGACC | ID NO: |
| cassette; (as shown in FIG. | ACGTCGCGTATGCGTGCCCGGATGCCGATG | 31 |
| 15B and FIG. 16) | AAGCTTCTAAGTATTACCAGGAAACATTCG | |
| ribosome binding sites are | GTTGGCACGAGTTGCACCGCGAAGAGAATC | |
| underlined | CAGAACAGGGCGTGGTGGAAATTATGATG | |
| | GCGCCTGCTGCGAAATTGACGGAGCACATG | |
| | ACTCAGGTGCAAGTTATGGCGCCTTTGAAC | |
| | GATGAGAGTACGGTCGCGAAGTGGCTTGC | |
| | GAAACACAATGGGCGTGCTGGATTGCACCA | |
| | CATGGCATGGCGTGTTGATGACATCGACGC | |
| | AGTGTCCGCAACACTTCGCGAGCGCGGTGT | |
| | ACAGTTGCTTTACGACGAGCCGAAACTGGG | |
| | TACAGGTGGGAATCGTATCAACTTCATGCA | |
| | TCCGAAATCTGGTAAAGGCGTGCTGATTGA | |
| | ACTGACCCAGTACCCCAAGAATTGATAAAG | |
| | GTTTTTCCTAAGACGCTAGCGCATAAGGTCCA | |
| | CCAAATGTCAAGTACAGACCAAGGCACGAA | |
| | CCCTGCTGACACGGATGATTTAACGCCAAC | |
| | CACATTATCCCTGGCTGGTGATTTCCCTAA | |
| | GGCTACGGAAGAGCAGTGGGAGCGCGAGG | |
| | TTGAAAAGGTGTTGAACCGTGGGCGCCCAC | |
| | CCGAGAAGCAGTTGACGTTTGCTGAATGTT | |
| | TAAAACGTCTTACTGTGCACACAGTAGATG | |
| | GCATTGACATCGTTCCAATGTATCGCCCGA | |
| | AGGATGCCCCTAAGAAACTGGGGTATCCAG | |
| | GGGTTGCTCCCTTTACGCGTGGCACTACGG | |
| | TTCGCAATGGGGATATGGACGCTTGGGACG | |
| | TTCGCGCCCTGCACGAAGACCCTGATGAAA | |
| | AATTCACGCGCAAAGCTATTCTGGAGGGGC | |
| | TGGAGCGCGGCGTAACAAGTTTGCTTCTTC | |
| | GTGTGGACCCTGATGCAATCGCTCCCGAAC | |
| | ACTTAGACGAAGTGTTAAGTGACGTTTTGC | |
| | TGGAAATGACCAAGGTTGAGGTGTTTTCCC | |
| | GCTATGATCAGGGAGCTGCGGCTGAAGCTC | |
| | TTGTCTCGGTATATGAGCGCAGCGACAAAC | |
| | CGGCTAAAGATTTGGCCTTAAATTTGGGAC | |
| | TGGACCCAATCGCATTTGCTGCACTTCAGG | |
| | GCACTGAGCCAGACTTGACCGTACTTGGTG | |
| | ATTGGGTTCGTCGTTTGGCTAAATTCAGCC | |
| | CAGACTCACGCGCTGTAACAATTGATGCTA | |
| | ATATTTATCACAACGCCGGTGCAGGCGACG | |
| | TTGCCGAGCTGGCCTGGGCACTTGCGACCG | |
| | GAGCAGAGTACGTCCGTGCGCTGGTAGAG | |
| | CAAGGATTCACCGCCACAGAGGCATTTGAT | |
| | ACCATTAACTTCCGTGTGACAGCGACCCAT | |
| | GATCAATTTTTAACGATTGCCCGCCTTCGT | |
| | GCGTTACGTGAAGCGTGGGCTCGTATCGGT | |
| | GAGGTATTCGGAGTAGATGAGGATAAACGT | |
| | GGAGCGCGCCAGAATGCTATTACGTCCTGG | |
| | CGTGAACTGACACGCGAGGATCCCTATGTG | |
| | AACATTTTACGTGGAAGTATTGCCACGTTC | |
| | TCTGCGTCCGTTGGGGGCGCGGAGTCTATT | |
| | ACCACTTTGCCATTCACGCAGGCATTGGGC | |
| | CTTCCAGAGGATGATTTTCCATTACGTATC | |
| | GCACGTAATACAGGAATTGTCTTAGCTGAG | |
| | GAGGTAAACATTGGGCGTGTAAATGACCCT | |
| | GCCGGGGGGTCATACTATGTGGAGAGCTT | |
| | GACTCGTTCTCTTGCAGATGCAGCATGGAA | |
| | AGAGTTCCAAGAGGTTGAAAAGTTGGGTGG | |
| | TATGTCTAAGGCCGTCATGACCGAACACGT | |
| | CACGAAGGTTTTAGATGCTTGCAACGCAGA | |
| | GCGCGCGAAGCGCTTGGCCAACCGCAAGC | |
| | AACCTATTACGGCAGTTTCCGAATTTCCGA | |
| | TGATTGGCGCACGCAGCATTGAGACGAAAC | |
| | CATTTCCGGCTGCTCCGGCCCGTAAAGGGC | |
| | TGGCATGGCACCGCGATTCCGAAGTCTTCG | |
| | AGCAACTTATGGACCGCTCCACGTCAGTTT | |
| | CAGAGCGTCCGAAAGTATTTTTAGCATGTC | |
| | TTGGGACGCGCCGCGATTTTGGAGGACGC | |
| | GAAGGATTTTCATCTCCGGTTTGGCACATT | |
| | GCCGGGATTGACACGCCTCAAGTAGAAGGT | |
| | GGGACGACTGCTGAAATCGTGGAAGCGTTC | |
| | AAAAAATCTGGGGCCCAAGTCGCCGATTTA | |
| | TGTTCGAGTGCCAAAGTGTATGCTCAACAA | |
| | GGCTTAGAGGTGGCAAAGGCTCTGAAAGC | |
| | GGCTGGGGCTAAGGCGCTGTATTTGAGCG | |
| | GAGCATTTAAGGAGTTCGGAGACGATGCAG | |
| | CGGAAGCCGAAAAACTTATCGACGGACGCC | |
| | TTTTCATGGGCATGGATGTCGTTGACACCC | |
| | TGTCTTCCACTTTAGATATCCTTGGAGTGG | |
| | CGAAGTGATAAGCTTAAAACAATTTACATCC | |
| | GGCCGGAACTTACTATGTCTACCTTACCTCG | |
| | CTTTGACAGTGTTGATTTAGGAAATGCGCC | |
| | GGTCCCAGCAGATGCTGCACGTCGTTTTGA | |
| | GGAACTTGCGGCGAAAGCCGGGACCGGCG | |
| | AAGCCTGGGAAACTGCGGAACAAATTCCAG | |
| | TAGGCACGTTGTTTAATGAAGACGTATACA | |
| | AGGACATGGATTGGCTTGATACTTACGCTG | |
| | GCATTCCTCCCTTCGTCCATGGTCCGTACG | |
| | CTACTATGTATGCATTTCGTCCTTGGACCA | |
| | TTCGCCAATATGCCGGTTTTTCGACTGCAA | |
| | AGGAGTCAAACGCATTTTACCGTCGTAATT | |
| | TGGCTGCAGGCCAGAAAGGTCTTAGTGTTG | |
| | CTTTTGACTTACCCACTCACCGCGGTTATG | |
| | ATTCCGACAACCCCCGCGTGGCCGGAGATG | |
| | TTGGTATGGCCGGTGTGGCTATCGATTCGA | |
| | TTTATGACATGCGTGAGCTGTTCGCCGGCA | |
| | TCCCATTAGATCAGATGAGCGTGTCGATGA | |
| | CAATGAACGGTGCTGTCTTGCCGATTTTGG | |
| | CTCTTTATGTGGTTACGGCGGAGGAGCAAG | |
| | GCGTGAAGCCAGAACAACTGGCGGGTACT | |
| | ATTCAAAATGATATTCTGAAGGAATTTATG | |
| | GTTCGTAATACATATATTTACCCGCCGCAA | |
| | CCTAGTATGCGCATTATCAGCGAGATTTTT | |
| | GCATACACATCAGCAAACATGCCGAAGTGG | |
| | AACTCCATTAGTATCAGCGGCTATCATATG | |
| | CAGGAGGCTGGAGCGACTGCGGATATCGA | |
| | GATGGCGTATACCTTAGCTGATGGAGTTGA | |
| | TTACATCCGTGCTGGTGAGTCAGTAGGACT | |
| | TAATGTGGACCAATTTGCTCCACGCCTGTC | |
| | CTTCTTCTGGGGCATTGGTATGAACTTTTT | |
| | CATGGAGGTAGCGAAGTTACGCGCTGCCC | |
| | GTATGCTGTGGGCGAAGCTTGTCCACCAGT | |
| | TCGGCCCGAAAAACCCGAAGAGTATGTCTC | |
| | TGCGCACGCACTCTCAAACATCGGGTTGGT | |
| | CTTTGACAGCTCAAGACGTATATAATAACG | |
| | TTGTACGTACATGCATCGAAGCCATGGCTG | |
| | CTACTCAAGGCCATACTCAATCACTTCATA | |
| | CAAATTCGTTGGATGAAGCCATTGCATTGC | |
| | CTACGGACTTTTCAGCCCGCATTGCCCGCA | |
| | ATACTCAATTATTTCTGCAACAAGAGAGCG | |
| | GGACGACTCGTGTGATCGACCCTTGGTCAG | |
| | GTTCCGCATACGTCGAAGAGTTGACTTGGG | |
| | ATTTAGCTCGTAAAGCCTGGGGGCATATTC | |
| | AGGAGGTTGAGAAGGTGGGGGGCATGGCT | |
| | AAGGCAATCGAGAAGGGGATTCCGAAGAT | |
| | GCGCATTGAGGAGGCAGCCGCCCGTACCC | |
| | AAGCACGTATTGATTCGGGACGCCAGCCAT | |
| | TAATTGGGGTCAATAAATACCGTCTGGAGC | |
| | ACGAACCACCCCTGGATGTGTTGAAGGTAG | |
| | ACAATAGCACCGTGTTAGCTGAGCAAAAGG | |
| | CCAAACTTGTTAAATTGCGCGCAGAACGCG | |
| | ACCCAGAAAAGGTCAAGGCTGCTCTGGACA | |
| | AAATCACTTGGGCGGCTGGCAATCCTGATG | |
| | ATAAAGACCCTGATCGCAACTTATTAAAGC | |
| | TGTGCATTGATGCGGGGCGCGCGATGGCA | |
| | ACGGTAGGAGAGATGAGTGACGCTTTAGA | |
| | GAAAGTTTTTGGGCGCTACACAGCGCAAAT | |
| | TCGCACTATTTCAGGAGTATATTCAAAAGA | |
| | AGTCAAAAACACTCCGGAAGTCGAGGAGG | |
| | CTCGCGAACTGGTAGAAGAGTTTGAGCAGG | |
| | CCGAAGGCCGTCGCCCACGTATCCTGCTGG | |
| | CTAAAATGGGGCAGGACGGTCATGACCGT | |
| | GGGCAAAAGGTCATCGCGACTGCATACGCC | |
| | GATTTGGGATTTGACGTGGACGTTGGCCCG | |
| | TTATTCCAAACTCCCGAGGAAACTGCTCGC | |
| | CAAGCCGTCGAAGCCGATGTGCACGTAGTG | |
| | GGGGTGAGCTCTCTGGCGGGAGGGCATCT | |
| | TACGCTTGTGCCTGCGCTTCGCAAAGAGCT | |
| | GGACAAGTTGGGTCGTCCAGATATTCTGAT | |
| | TACCGTAGGAGGGGTTATTCCCGAGCAGGA | |
| | CTTCGATGAGCTTCGTAAGGATGGCGCTGT | |
| | TGAAATCTACACACCGGGGACGGTCATTCC | |
| | AGAATCGGCTATCTCTTTAGTTAAAAAATT | |
| | GCGCGCCTCCCTGGATGCT | |
| mmcE sequence | ATGAGTAACGAAGATTTATTCATTTGCATCGA | SEQ |
| (comprised in the mmcE- | CCACGTCGCGTATGCGTGCCCGGATGCCGATG | ID NO: |
| mutA-mutB construct | AAGCTTCTAAGTATTACCAGGAAACATTCGGT | 32 |
| shown in FIG. 15B and | TGGCACGAGTTGCACCGCGAAGAGAATCCAG | |
| FIG. 16) | AACAGGGCGTGGTGGAAATTATGATGGCGCC | |
| | TGCTGCGAAATTGACGGAGCACATGACTCAG | |
| | GTGCAAGTTATGGCGCCTTTGAACGATGAGAG | |
| | TACGGTCGCGAAGTGGCTTGCGAAACACAAT | |
| | GGGCGTGCTGGATTGCACCACATGGCATGGC | |
| | GTGTTGATGACATCGACGCAGTGTCCGCAACA | |
| | CTTCGCGAGCGCGGTGTACAGTTGCTTTACGA | |
| | CGAGCCGAAACTGGGTACAGGTGGGAATCGT | |
| | ATCAACTTCATGCATCCGAAATCTGGTAAAGG | |
| | CGTGCTGATTGAACTGACCCAGTACCCCAAGA | |
| | ATTGA | |
| mutA sequence (comprised | ATGTCAAGTACAGACCAAGGCACGAACCCTG | SEQ |
| in the mmcE-mutA-mutB | CTGACACGGATGATTTAACGCCAACCACATTA | ID NO: |
| construct shown in FIG. | TCCCTGGCTGGTGATTTCCCTAAGGCTACGGA | 33 |
| 15B and FIG. 16) | AGAGCAGTGGGAGCGCGAGGTTGAAAAGGTG | |
| | TTGAACCGTGGGCGCCCACCCGAGAAGCAGT | |
| | TGACGTTTGCTGAATGTTTAAAACGTCTTACT | |
| | GTGCACACAGTAGATGGCATTGACATCGTTCC | |
| | AATGTATCGCCCGAAGGATGCCCCTAAGAAA | |
| | CTGGGGTATCCAGGGGTTGCTCCCTTTACGCG | |
| | TGGCACTACGGTTCGCAATGGGGATATGGAC | |
| | GCTTGGGACGTTCGCGCCCTGCACGAAGACCC | |
| | TGATGAAAAATTCACGCGCAAAGCTATTCTGG | |
| | AGGGGCTGGAGCGCGGCGTAACAAGTTTGCT | |
| | TCTTCGTGTGGACCCTGATGCAATCGCTCCCG | |
| | AACACTTAGACGAAGTGTTAAGTGACGTTTTG | |
| | CTGGAAATGACCAAGGTTGAGGTGTTTTCCCG | |
| | CTATGATCAGGGAGCTGCGGCTGAAGCTCTTG | |
| | TCTCGGTATATGAGCGCAGCGACAAACCGGCT | |
| | AAAGATTTGGCCTTAAATTTGGGACTGGACCC | |
| | AATCGCATTTGCTGCACTTCAGGGCACTGAGC | |
| | CAGACTTGACCGTACTTGGTGATTGGGTTCGT | |
| | CGTTTGGCTAAATTCAGCCCAGACTCACGCGC | |
| | TGTAACAATTGATGCTAATATTTATCACAACG | |
| | CCGGTGCAGGCGACGTTGCCGAGCTGGCCTG | |
| | GGCACTTGCGACCGGAGCAGAGTACGTCCGT | |
| | GCGCTGGTAGAGCAAGGATTCACCGCCACAG | |
| | AGGCATTTGATACCATTAACTTCCGTGTGACA | |
| | GCGACCCATGATCAATTTTTAACGATTGCCCG | |
| | CCTTCGTGCGTTACGTGAAGCGTGGGCTCGTA | |
| | TCGGTGAGGTATTCGGAGTAGATGAGGATAA | |
| | ACGTGGAGCGCGCCAGAATGCTATTACGTCCT | |
| | GGCGTGAACTGACACGCGAGGATCCCTATGT | |
| | GAACATTTTACGTGGAAGTATTGCCACGTTCT | |
| | CTGCGTCCGTTGGGGGCGCGGAGTCTATTACC | |
| | ACTTTGCCATTCACGCAGGCATTGGGCCTTCC | |
| | AGAGGATGATTTTCCATTACGTATCGCACGTA | |
| | ATACAGGAATTGTCTTAGCTGAGGAGGTAAA | |
| | CATTGGGCGTGTAAATGACCCTGCCGGGGGGT | |
| | CATACTATGTGGAGAGCTTGACTCGTTCTCTT | |
| | GCAGATGCAGCATGGAAAGAGTTCCAAGAGG | |
| | TTGAAAAGTTGGGTGGTATGTCTAAGGCCGTC | |
| | ATGACCGAACACGTCACGAAGGTTTTAGATGC | |
| | TTGCAACGCAGAGCGCGCGAAGCGCTTGGCC | |
| | AACCGCAAGCAACCTATTACGGCAGTTTCCGA | |
| | ATTTCCGATGATTGGCGCACGCAGCATTGAGA | |
| | CGAAACCATTTCCGGCTGCTCCGGCCCGTAAA | |
| | GGGCTGGCATGGCACCGCGATTCCGAAGTCTT | |
| | CGAGCAACTTATGGACCGCTCCACGTCAGTTT | |
| | CAGAGCGTCCGAAAGTATTTTTAGCATGTCTT | |
| | GGGACGCGCCGCGATTTTGGAGGACGCGAAG | |
| | GATTTTCATCTCCGGTTTGGCACATTGCCGGG | |
| | ATTGACACGCCTCAAGTAGAAGGTGGGACGA | |
| | CTGCTGAAATCGTGGAAGCGTTCAAAAAATCT | |
| | GGGGCCCAAGTCGCCGATTTATGTTCGAGTGC | |
| | CAAAGTGTATGCTCAACAAGGCTTAGAGGTG | |
| | GCAAAGGCTCTGAAAGCGGCTGGGGCTAAGG | |
| | CGCTGTATTTGAGCGGAGCATTTAAGGAGTTC | |
| | GGAGACGATGCAGCGGAAGCCGAAAAACTTA | |
| | TCGACGGACGCCTTTTCATGGGCATGGATGTC | |
| | GTTGACACCCTGTCTTCCACTTTAGATATCCTT | |
| | GGAGTGGCGAAGTGA | |
| mutB sequence (comprised | ATGTCTACCTTACCTCGCTTTGACAGTGTTGAT | SEQ |
| in the mmcE-mutA-mutB | TTAGGAAATGCGCCGGTCCCAGCAGATGCTGC | ID NO: |
| construct shown in FIG. | ACGTCGTTTTGAGGAACTTGCGGCGAAAGCCG | 34 |
| 15B and FIG. 16) | GGACCGGCGAAGCCTGGGAAACTGCGGAACA | |
| | AATTCCAGTAGGCACGTTGTTTAATGAAGACG | |
| | TATACAAGGACATGGATTGGCTTGATACTTAC | |
| | GCTGGCATTCCTCCCTTCGTCCATGGTCCGTA | |
| | CGCTACTATGTATGCATTTCGTCCTTGGACCA | |
| | TTCGCCAATATGCCGGTTTTTCGACTGCAAAG | |
| | GAGTCAAACGCATTTTACCGTCGTAATTTGGC | |
| | TGCAGGCCAGAAAGGTCTTAGTGTTGCTTTTG | |
| | ACTTACCCACTCACCGCGGTTATGATTCCGAC | |
| | AACCCCCGCGTGGCCGGAGATGTTGGTATGGC | |
| | CGGTGTGGCTATCGATTCGATTTATGACATGC | |
| | GTGAGCTGTTCGCCGGCATCCCATTAGATCAG | |
| | ATGAGCGTGTCGATGACAATGAACGGTGCTGT | |
| | CTTGCCGATTTTGGCTCTTTATGTGGTTACGGC | |
| | GGAGGAGCAAGGCGTGAAGCCAGAACAACTG | |
| | GCGGGTACTATTCAAAATGATATTCTGAAGGA | |
| | ATTTATGGTTCGTAATACATATATTTACCCGC | |
| | CGCAACCTAGTATGCGCATTATCAGCGAGATT | |
| | TTTGCATACACATCAGCAAACATGCCGAAGTG | |
| | GAACTCCATTAGTATCAGCGGCTATCATATGC | |
| | AGGAGGCTGGAGCGACTGCGGATATCGAGAT | |
| | GGCGTATACCTTAGCTGATGGAGTTGATTACA | |
| | TCCGTGCTGGTGAGTCAGTAGGACTTAATGTG | |
| | GACCAATTTGCTCCACGCCTGTCCTTCTTCTGG | |
| | GGCATTGGTATGAACTTTTTCATGGAGGTAGC | |
| | GAAGTTACGCGCTGCCCGTATGCTGTGGGCGA | |
| | AGCTTGTCCACCAGTTCGGCCCGAAAAACCCG | |
| | AAGAGTATGTCTCTGCGCACGCACTCTCAAAC | |
| | ATCGGGTTGGTCTTTGACAGCTCAAGACGTAT | |
| | ATAATAACGTTGTACGTACATGCATCGAAGCC | |
| | ATGGCTGCTACTCAAGGCCATACTCAATCACT | |
| | TCATACAAATTCGTTGGATGAAGCCATTGCAT | |
| | TGCCTACGGACTTTTCAGCCCGCATTGCCCGC | |
| | AATACTCAATTATTTCTGCAACAAGAGAGCGG | |
| | GACGACTCGTGTGATCGACCCTTGGTCAGGTT | |
| | CCGCATACGTCGAAGAGTTGACTTGGGATTTA | |
| | GCTCGTAAAGCCTGGGGGCATATTCAGGAGG | |
| | TTGAGAAGGTGGGGGGCATGGCTAAGGCAAT | |
| | CGAGAAGGGGATTCCGAAGATGCGCATTGAG | |
| | GAGGCAGCCGCCCGTACCCAAGCACGTATTG | |
| | ATTCGGGACGCCAGCCATTAATTGGGGTCAAT | |
| | AAATACCGTCTGGAGCACGAACCACCCCTGG | |
| | ATGTGTTGAAGGTAGACAATAGCACCGTGTTA | |
| | GCTGAGCAAAAGGCCAAACTTGTTAAATTGC | |
| | GCGCAGAACGCGACCCAGAAAAGGTCAAGGC | |
| | TGCTCTGGACAAAATCACTTGGGCGGCTGGCA | |
| | ATCCTGATGATAAAGACCCTGATCGCAACTTA | |
| | TTAAAGCTGTGCATTGATGCGGGGCGCGCGAT | |
| | GGCAACGGTAGGAGAGATGAGTGACGCTTTA | |
| | GAGAAAGTTTTTGGGCGCTACACAGCGCAAA | |
| | TTCGCACTATTTCAGGAGTATATTCAAAAGAA | |
| | GTCAAAAACACTCCGGAAGTCGAGGAGGCTC | |
| | GCGAACTGGTAGAAGAGTTTGAGCAGGCCGA | |
| | AGGCCGTCGCCCACGTATCCTGCTGGCTAAAA | |
| | TGGGGCAGGACGGTCATGACCGTGGGCAAAA | |
| | GGTCATCGCGACTGCATACGCCGATTTGGGAT | |
| | TTGACGTGGACGTTGGCCCGTTATTCCAAACT | |
| | CCCGAGGAAACTGCTCGCCAAGCCGTCGAAG | |
| | CCGATGTGCACGTAGTGGGGGTGAGCTCTCTG | |
| | GCGGGAGGGCATCTTACGCTTGTGCCTGCGCT | |
| | TCGCAAAGAGCTGGACAAGTTGGGTCGTCCA | |
| | GATATTCTGATTACCGTAGGAGGGGTTATTCC | |
| | CGAGCAGGACTTCGATGAGCTTCGTAAGGAT | |
| | GGCGCTGTTGAAATCTACACACCGGGGACGG | |
| | TCATTCCAGAATCGGCTATCTCTTTAGTTAAA | |
| | AAATTGCGCGCCTCCCTGGATGCT | |
| Construct comprising TetR | Ttaagacccactttcacatttaagttgtttttctaatccgcatatgatcaattcaa | SEQ |
| (reverse orientation, | ggccgaataagaaggctggctctgcaccttggtgatcaaataattcgatagc | ID NO: |
| lowercase) and prpE-accA- | ttgtcgtaataatggcggcatactatcagtagtaggtgtttccctttcttctttag | 35 |
| pccB gene cassette driven | cgacttgatgctcttgatcttccaatacgcaacctaaagtaaaatgccccaca | |
| by tet inducible promoter | gcgctgagtgcatataatgcattctctagtgaaaaaccttgttggcataaaaa | |
| (italics) (as shown in FIG. | ggctaattgattttcgagagtttcatactgtttttctgtaggccgtgtacctaaat | |
| 15B and FIG. 16); | gtacttttgctccatcgcgatgacttagtaaagcacatctaaaacttttagcgtt | |
| ribosome binding sites are | attacgtaaaaaatcttgccagctttccccttctaaagggcaaaagtgagtat | |
| underlined; coding | ggtgcctatctaacatctcaatggctaaggcgtcgagcaaagcccgcttattt | |
| sequences bold and | tttacatgccaatacaatgtaggctgctctacacctagcttctgggcgagttta | |
| underlined | cgggttgttaaaccttcgattccgacctcattaagcagctctaatgcgctgtta | |
| | atcactttacttttatctaatctagacatcatTAATTCCTAATTTTTG | |
| | TTGACACTCTATCATTGATAGAGTTATTTTACCAC | |
| | TCCCTATCAGTGATAGAGAAAAGTGAATAAGGCG | |
| | TAAGTTCAACAGGAGAGCATTTAAGGCGTAAGT | |
| | TCAACAGGAGAGCATTATGTCTTTTAGCGAA | |
| | TTTTATCAGCGTTCGATTAACGAACCGGAG | |
| | AAGTTCTGGGCCGAGCAGGCCCGGCGTATT | |
| | GACTGGCAGACGCCCTTTACGCAAACGCTC | |
| | GACCACAGCAACCCGCCGTTTGCCCGTTGG | |
| | TTTTGTGAAGGCCGAACCAACTTGTGTCAC | |
| | AACGCTATCGACCGCTGGCTGGAGAAACAG | |
| | CCAGAGGCGCTGGCATTGATTGCCGTCTCT | |
| | TCGGAAACAGAGGAAGAGCGTACCTTTACC | |
| | TTCCGCCAGTTACATGACGAAGTGAATGCG | |
| | GTGGCGTCAATGCTGCGCTCACTGGGCGTG | |
| | CAGCGTGGCGATCGGGTGCTGGTGTATATG | |
| | CCGATGATTGCCGAAGCGCATATTACCCTG | |
| | CTGGCCTGCGCGCGCATTGGTGCTATTCAC | |
| | TCGGTGGTGTTTGGGGGATTTGCTTCGCAC | |
| | AGCGTGGCAACGCGAATTGATGACGCTAAA | |
| | CCGGTGCTGATTGTCTCGGCTGATGCCGGG | |
| | GCGCGCGGCGGTAAAATCATTCCGTATAAA | |
| | AAATTGCTCGACGATGCGATAAGTCAGGCA | |
| | CAGCATCAGCCGCGTCACGTTTTACTGGTG | |
| | GATCGCGGGCTGGCGAAAATGGCGCGCGT | |
| | TAGCGGGCGGGATGTCGATTTCGCGTCGTT | |
| | GCGCCATCAACACATCGGCGCGCGGGTGC | |
| | CGGTGGCATGGCTGGAATCCAACGAAACCT | |
| | CCTGCATTCTCTACACCTCCGGCACGACCG | |
| | GCAAACCTAAAGGTGTGCAGCGTGATGTCG | |
| | GCGGATATGCGGTGGCGCTGGCGACCTCG | |
| | ATGGACACCATTTTTGGCGGCAAAGCGGGC | |
| | GGCGTGTTCTTTTGTGCTTCGGATATCGGC | |
| | TGGGTGGTAGGGCATTCGTATATCGTTTAC | |
| | GCGCCGCTGCTGGCGGGGATGGCGACTAT | |
| | CGTTTACGAAGGATTGCCGACCTGGCCGGA | |
| | CTGCGGCGTGTGGTGGAAAATTGTCGAGAA | |
| | ATATCAGGTTAGCCGCATGTTCTCAGCGCC | |
| | GACCGCCATTCGCGTGCTGAAAAAATTCCC | |
| | TACCGCTGAAATTCGCAAACACGATCTTTC | |
| | GTCGCTGGAAGTGCTCTATCTGGCTGGAGA | |
| | ACCGCTGGACGAGCCGACCGCCAGTTGGG | |
| | TGAGCAATACGCTGGATGTGCCGGTCATCG | |
| | ACAACTACTGGCAGACCGAATCCGGCTGGC | |
| | CGATTATGGCGATTGCTCGCGGTCTGGATG | |
| | ACAGACCGACGCGTCTGGGAAGCCCCGGC | |
| | GTGCCGATGTATGGCTATAACGTGCAGTTG | |
| | CTCAATGAAGTCACCGGCGAACCGTGTGGC | |
| | GTCAATGAGAAAGGGATGCTGGTAGTGGA | |
| | GGGGCCATTGCCGCCAGGCTGTATTCAAAC | |
| | CATCTGGGGCGACGACGACCGCTTTGTGAA | |
| | GACGTACTGGTCGCTGTTTTCCCGTCCGGT | |
| | GTACGCCACTTTTGACTGGGGCATCCGCGA | |
| | TGCTGACGGTTATCACTTTATTCTCGGGCG | |
| | CACTGACGATGTGATTAACGTTGCCGGACA | |
| | TCGGCTGGGTACGCGTGAGATTGAAGAGA | |
| | GTATCTCCAGTCATCCGGGCGTTGCCGAAG | |
| | TGGCGGTGGTTGGGGTGAAAGATGCGCTG | |
| | AAAGGGCAGGTGGCGGTGGCGTTTGTCATT | |
| | CCGAAAGAGAGCGACAGTCTGGAAGACCG | |
| | TGAGGTGGCGCACTCGCAAGAGAAGGCGA | |
| | TTATGGCGCTGGTGGACAGCCAGATTGGCA | |
| | ACTTTGGCCGCCCGGCGCACGTCTGGTTTG | |
| | TCTCGCAATTGCCAAAAACGCGATCCGGAA | |
| | AAATGCTGCGCCGCACGATCCAGGCGATTT | |
| | GCGAAGGACGCGATCCTGGGGATCTGACG | |
| | ACCATTGATGATCCGGCGTCGTTGGATCAG | |
| | ATCCGCCAGGCGATGGAAGAGTAGTACTAG | |
| | ATTCAATATAGAGTAAAAGAGGTAAGAGTAT | |
| | CCATGCGTAAAGTTCTGATCGCTAATCGTG | |
| | GAGAAATTGCTGTACGTGTAGCACGTGCAT | |
| | GTCGTGATGCGGGAATCGCATCAGTAGCCG | |
| | TATACGCGGACCCGGATCGTGACGCGTTGC | |
| | ATGTGCGCGCGGCGGACGAAGCATTTGCA | |
| | CTGGGTGGTGATACGCCTGCAACATCTTAC | |
| | TTAGACATCGCCAAGGTGTTAAAGGCTGCA | |
| | CGTGAGAGTGGTGCAGACGCCATTCATCCC | |
| | GGTTACGGCTTTTTAAGTGAAAATGCCGAG | |
| | TTCGCGCAGGCCGTGTTAGATGCGGGTCTT | |
| | ATCTGGATCGGACCACCGCCCCATGCAATC | |
| | CGCGATCGTGGGGAAAAAGTTGCAGCTCG | |
| | CCATATTGCCCAGCGTGCTGGGGCGCCGCT | |
| | GGTTGCGGGCACCCCTGACCCGGTTTCTGG | |
| | TGCTGACGAAGTCGTCGCCTTCGCGAAAGA | |
| | GCATGGACTGCCGATCGCGATTAAGGCTGC | |
| | TTTTGGAGGCGGTGGTCGTGGTTTAAAGGT | |
| | TGCCCGTACATTGGAAGAAGTGCCCGAGTT | |
| | ATATGACTCCGCCGTGCGTGAAGCTGTGGC | |
| | GGCATTCGGACGTGGCGAATGTTTCGTGGA | |
| | GCGCTATTTAGACAAACCGCGTCATGTAGA | |
| | AACCCAGTGCTTGGCAGATACTCACGGTAA | |
| | TGTAGTTGTGGTTTCTACTCGCGACTGTTC | |
| | GTTACAGCGTCGTCATCAGAAACTGGTAGA | |
| | GGAGGCACCCGCCCCGTTTTTAAGCGAAGC | |
| | TCAGACAGAGCAACTGTACTCCTCCTCCAA | |
| | GGCTATTCTTAAGGAAGCTGGGTATGGTGG | |
| | AGCGGGAACCGTTGAGTTTTTAGTAGGTAT | |
| | GGATGGTACTATCTTCTTCTTGGAGGTCAA | |
| | TACCCGCCTGCAGGTGGAGCACCCTGTGAC | |
| | CGAAGAAGTCGCAGGGATCGACCTGGTCC | |
| | GTGAAATGTTCCGCATTGCAGATGGCGAGG | |
| | AGCTGGGGTACGACGATCCAGCCCTTCGCG | |
| | GCCACTCGTTCGAATTTCGCATCAATGGGG | |
| | AGGACCCAGGTCGTGGTTTTTTGCCCGCAC | |
| | CTGGTACGGTTACGCTTTTTGATGCTCCGA | |
| | CCGGACCCGGAGTCCGCCTGGATGCCGGG | |
| | GTTGAGTCAGGTTCCGTAATCGGACCGGCA | |
| | TGGGACTCACTGCTGGCTAAACTTATCGTT | |
| | ACCGGGCGTACACGTGCCGAGGCGCTTCA | |
| | GCGCGCAGCCCGCGCCTTAGATGAATTTAC | |
| | GGTTGAGGGCATGGCAACCGCGATCCCTTT | |
| | CCATCGCACAGTAGTACGCGATCCAGCATT | |
| | CGCTCCTGAGCTTACCGGGTCAACGGACCC | |
| | ATTCACCGTTCATACACGCTGGATTGAAAC | |
| | TGAATTTGTCAACGAAATTAAGCCTTTTAC | |
| | CACCCCTGCCGACACGGAGACAGATGAAG | |
| | AGTCTGGGCGCGAGACAGTGGTAGTCGAG | |
| | GTCGGTGGGAAACGCTTAGAGGTAAGTCTT | |
| | CCGTCCAGCCTGGGAATGTCGTTGGCCCGT | |
| | ACCGGCCTTGCCGCGGGGGCCCGCCCCAA | |
| | ACGCCGCGCGGCCAAGAAGTCAGGCCCTG | |
| | CAGCATCGGGTGATACACTGGCATCTCCTA | |
| | TGCAAGGTACGATCGTAAAGATCGCCGTGG | |
| | AAGAGGGACAAGAAGTACAGGAGGGAGATCT | |
| | GATTGTGGTTCTTGAAGCTATGAAGATGGAAC | |
| | AGCCACTTAATGCCCACCGTTCGGGAACCATT | |
| | AAGGGGCTTACTGCTGAAGTAGGTGCTTCACT | |
| | GACGTCGGGCGCCGCTATCTGTGAAATCAAG | |
| | GATTGATAACGCTAACGAAAAAGTTAAATAC | |
| | AGGAACAAGAGAACATATGTCGGAGCCCGA | |
| | GGAACAGCAGCCAGATATCCACACGACAGC | |
| | GGGCAAGTTAGCTGATCTTCGTCGCCGCAT | |
| | CGAAGAGGCAACGCACGCCGGTTCTGCGC | |
| | GCGCGGTGGAGAAACAGCACGCGAAGGGT | |
| | AAACTTACGGCTCGTGAGCGTATCGATTTG | |
| | TTGCTGGACGAAGGGTCTTTTGTAGAGCTT | |
| | GATGAGTTTGCGCGTCACCGTTCGACGAAT | |
| | TTCGGACTGGATGCCAACCGTCCATATGGA | |
| | GATGGAGTGGTGACTGGCTATGGAACTGTT | |
| | GACGGACGTCCGGTTGCCGTCTTTTCGCAA | |
| | GACTTTACGGTCTTTGGGGGCGCTCTGGGG | |
| | GAAGTATACGGGCAAAAAATTGTGAAGGTC | |
| | ATGGATTTCGCTCTTAAGACCGGGTGTCCC | |
| | GTCGTGGGTATTAATGACTCAGGTGGGGCA | |
| | CGCATTCAAGAGGGTGTAGCAAGTCTGGGC | |
| | GCGTATGGAGAGATTTTCCGTCGCAATACG | |
| | CACGCGTCGGGCGTGATCCCTCAGATTTCG | |
| | CTTGTAGTTGGCCCATGCGCAGGGGGAGCT | |
| | GTGTACTCTCCAGCTATTACTGACTTTACG | |
| | GTAATGGTCGACCAAACATCGCATATGTTT | |
| | ATCACCGGACCCGATGTGATTAAGACAGTG | |
| | ACAGGGGAGGATGTGGGTTTTGAGGAACTT | |
| | GGTGGTGCGCGTACGCACAACAGTACGTCT | |
| | GGGGTTGCCCATCATATGGCTGGGGATGA | |
| | GAAAGACGCTGTGGAGTATGTTAAGCAATT | |
| | ATTGAGTTATTTGCCGTCGAACAATTTAAG | |
| | TGAGCCTCCGGCGTTTCCTGAAGAGGCTGA | |
| | TTTAGCCGTTACGGACGAAGATGCGGAATT | |
| | AGATACAATTGTGCCGGATTCGGCTAACCA | |
| | ACCCTATGATATGCATTCTGTAATCGAGCA | |
| | TGTCCTTGACGATGCGGAATTTTTCGAGAC | |
| | TCAACCGTTGTTTGCCCCCAACATCCTGAC | |
| | CGGCTTTGGTCGCGTTGAAGGCCGTCCGGT | |
| | GGGTATCGTGGCGAATCAGCCGATGCAGTT | |
| | TGCTGGATGCTTAGATATCACTGCCTCAGA | |
| | AAAAGCTGCTCGTTTCGTTCGCACTTGCGA | |
| | CGCTTTCAACGTCCCTGTGCTTACGTTTGT | |
| | AGACGTCCCCGGGTTTTTACCGGGCGTAGA | |
| | TCAGGAGCATGACGGGATCATCCGCCGCG | |
| | GTGCGAAGTTGATTTTTGCCTATGCAGAAG | |
| | CGACCGTGCCGTTGATCACAGTAATCACGC | |
| | GCAAAGCCTTCGGAGGTGCGTATGACGTAA | |
| | TGGGCTCAAAACACCTTGGCGCTGACCTTA | |
| | ATCTGGCATGGCCCACGGCCCAAATCGCTG | |
| | TAATGGGCGCTCAAGGTGCTGTAAACATCC | |
| | TTCATCGTCGTACGATTGCAGATGCGGGGG | |
| | ACGATGCGGAAGCCACGCGCGCCCGTTTAA | |
| | TTCAAGAGTACGAGGATGCTTTATTAAATC | |
| | CCTATACTGCGGCTGAGCGCGGGTATGTAG | |
| | ACGCGGTCATCATGCCCTCAGATACTCGCC | |
| | GTCATATCGTACGTGGTTTACGCCAATTAC | |
| | GCACCAAGCGCGAGTCTTTACCCCCGAAAA | |
| | AGCACGGGAACATTCCCCTT | |
| | TGAGGAGGTCGGATAAGGCGCTCGCGCCGCA | |
| | TCCGACACCGTGCGCAGATGCCTGATGCGACG | |
| | CTGACGCGTCTTATCATGCCTCGCTCTCGAGT | |
| | CCCGTCAAGTCAGCGTAATGCTCTGCCAGTGT | |
| | TACAACCAATTAACCAATTCTGAT | |
| Construct comprising a | TAATTCCTAATTTTTGTTGACACTCTATCATTGATA | SEQ |
| prpE-accA-pccB gene | GAGTTATTTTACCACTCCCTATCAGTGATAGAGAA | ID NO: |
| cassette under the control | AAGTGAATAAGGCGTAAGTTCAACAGGAGAGCAT | 36 |
| of the Ptet promoter (as | TTAAGGCGTAAGTTCAACAGGAGAGCATTAT | |
| shown in FIG. 15B and | GTCTTTTAGCGAATTTTATCAGCGTTCGATT | |
| FIG. 16) ribosome binding | AACGAACCGGAGAAGTTCTGGGCCGAGCA | |
| sites are underlined;. | GGCCCGGCGTATTGACTGGCAGACGCCCTT | |
| L3S2P11 terminator in | TACGCAAACGCTCGACCACAGCAACCCGCC | |
| italics; his terminator in | GTTTGCCCGTTGGTTTTGTGAAGGCCGAAC | |
| bold; coding sequences | CAACTTGTGTCACAACGCTATCGACCGCTG | |
| bold and underlined | GCTGGAGAAACAGCCAGAGGCGCTGGCAT | |
| | TGATTGCCGTCTCTTCGGAAACAGAGGAAG | |
| | AGCGTACCTTTACCTTCCGCCAGTTACATG | |
| | ACGAAGTGAATGCGGTGGCGTCAATGCTGC | |
| | GCTCACTGGGCGTGCAGCGTGGCGATCGG | |
| | GTGCTGGTGTATATGCCGATGATTGCCGAA | |
| | GCGCATATTACCCTGCTGGCCTGCGCGCGC | |
| | ATTGGTGCTATTCACTCGGTGGTGTTTGGG | |
| | GGATTTGCTTCGCACAGCGTGGCAACGCGA | |
| | ATTGATGACGCTAAACCGGTGCTGATTGTC | |
| | TCGGCTGATGCCGGGGCGCGCGGCGGTAA | |
| | AATCATTCCGTATAAAAAATTGCTCGACGA | |
| | TGCGATAAGTCAGGCACAGCATCAGCCGCG | |
| | TCACGTTTTACTGGTGGATCGCGGGCTGGC | |
| | GAAAATGGCGCGCGTTAGCGGGCGGGATG | |
| | TCGATTTCGCGTCGTTGCGCCATCAACACA | |
| | TCGGCGCGCGGGTGCCGGTGGCATGGCTG | |
| | GAATCCAACGAAACCTCCTGCATTCTCTAC | |
| | ACCTCCGGCACGACCGGCAAACCTAAAGGT | |
| | GTGCAGCGTGATGTCGGCGGATATGCGGT | |
| | GGCGCTGGCGACCTCGATGGACACCATTTT | |
| | TGGCGGCAAAGCGGGCGGCGTGTTCTTTTG | |
| | TGCTTCGGATATCGGCTGGGTGGTAGGGCA | |
| | TTCGTATATCGTTTACGCGCCGCTGCTGGC | |
| | GGGGATGGCGACTATCGTTTACGAAGGATT | |
| | GCCGACCTGGCCGGACTGCGGCGTGTGGT | |
| | GGAAAATTGTCGAGAAATATCAGGTTAGCC | |
| | GCATGTTCTCAGCGCCGACCGCCATTCGCG | |
| | TGCTGAAAAAATTCCCTACCGCTGAAATTC | |
| | GCAAACACGATCTTTCGTCGCTGGAAGTGC | |
| | TCTATCTGGCTGGAGAACCGCTGGACGAGC | |
| | CGACCGCCAGTTGGGTGAGCAATACGCTG | |
| | GATGTGCCGGTCATCGACAACTACTGGCAG | |
| | ACCGAATCCGGCTGGCCGATTATGGCGATT | |
| | GCTCGCGGTCTGGATGACAGACCGACGCG | |
| | TCTGGGAAGCCCCGGCGTGCCGATGTATG | |
| | GCTATAACGTGCAGTTGCTCAATGAAGTCA | |
| | CCGGCGAACCGTGTGGCGTCAATGAGAAA | |
| | GGGATGCTGGTAGTGGAGGGGCCATTGCC | |
| | GCCAGGCTGTATTCAAACCATCTGGGGCGA | |
| | CGACGACCGCTTTGTGAAGACGTACTGGTC | |
| | GCTGTTTTCCCGTCCGGTGTACGCCACTTT | |
| | TGACTGGGGCATCCGCGATGCTGACGGTTA | |
| | TCACTTTATTCTCGGGCGCACTGACGATGT | |
| | GATTAACGTTGCCGGACATCGGCTGGGTAC | |
| | GCGTGAGATTGAAGAGAGTATCTCCAGTCA | |
| | TCCGGGCGTTGCCGAAGTGGCGGTGGTTG | |
| | GGGTGAAAGATGCGCTGAAAGGGCAGGTG | |
| | GCGGTGGCGTTTGTCATTCCGAAAGAGAGC | |
| | GACAGTCTGGAAGACCGTGAGGTGGCGCA | |
| | CTCGCAAGAGAAGGCGATTATGGCGCTGGT | |
| | GGACAGCCAGATTGGCAACTTTGGCCGCCC | |
| | GGCGCACGTCTGGTTTGTCTCGCAATTGCC | |
| | AAAAACGCGATCCGGAAAAATGCTGCGCCG | |
| | CACGATCCAGGCGATTTGCGAAGGACGCG | |
| | ATCCTGGGGATCTGACGACCATTGATGATC | |
| | CGGCGTCGTTGGATCAGATCCGCCAGGCG | |
| | ATGGAAGAGTAGTACTAGATTCAATATAGAG | |
| | TAAAAGAGGTAAGAGTATCCATGCGTAAAGT | |
| | TCTGATCGCTAATCGTGGAGAAATTGCTGT | |
| | ACGTGTAGCACGTGCATGTCGTGATGCGGG | |
| | AATCGCATCAGTAGCCGTATACGCGGACCC | |
| | GGATCGTGACGCGTTGCATGTGCGCGCGG | |
| | CGGACGAAGCATTTGCACTGGGTGGTGATA | |
| | CGCCTGCAACATCTTACTTAGACATCGCCA | |
| | AGGTGTTAAAGGCTGCACGTGAGAGTGGT | |
| | GCAGACGCCATTCATCCCGGTTACGGCTTT | |
| | TTAAGTGAAAATGCCGAGTTCGCGCAGGCC | |
| | GTGTTAGATGCGGGTCTTATCTGGATCGGA | |
| | CCACCGCCCCATGCAATCCGCGATCGTGGG | |
| | GAAAAAGTTGCAGCTCGCCATATTGCCCAG | |
| | CGTGCTGGGGCGCCGCTGGTTGCGGGCAC | |
| | CCCTGACCCGGTTTCTGGTGCTGACGAAGT | |
| | CGTCGCCTTCGCGAAAGAGCATGGACTGCC | |
| | GATCGCGATTAAGGCTGCTTTTGGAGGCGG | |
| | TGGTCGTGGTTTAAAGGTTGCCCGTACATT | |
| | GGAAGAAGTGCCCGAGTTATATGACTCCGC | |
| | CGTGCGTGAAGCTGTGGCGGCATTCGGAC | |
| | GTGGCGAATGTTTCGTGGAGCGCTATTTAG | |
| | ACAAACCGCGTCATGTAGAAACCCAGTGCT | |
| | TGGCAGATACTCACGGTAATGTAGTTGTGG | |
| | TTTCTACTCGCGACTGTTCGTTACAGCGTC | |
| | GTCATCAGAAACTGGTAGAGGAGGCACCC | |
| | GCCCCGTTTTTAAGCGAAGCTCAGACAGAG | |
| | CAACTGTACTCCTCCTCCAAGGCTATTCTT | |
| | AAGGAAGCTGGGTATGGTGGAGCGGGAAC | |
| | CGTTGAGTTTTTAGTAGGTATGGATGGTAC | |
| | TATCTTCTTCTTGGAGGTCAATACCCGCCT | |
| | GCAGGTGGAGCACCCTGTGACCGAAGAAG | |
| | TCGCAGGGATCGACCTGGTCCGTGAAATGT | |
| | TCCGCATTGCAGATGGCGAGGAGCTGGGG | |
| | TACGACGATCCAGCCCTTCGCGGCCACTCG | |
| | TTCGAATTTCGCATCAATGGGGAGGACCCA | |
| | GGTCGTGGTTTTTTGCCCGCACCTGGTACG | |
| | GTTACGCTTTTTGATGCTCCGACCGGACCC | |
| | GGAGTCCGCCTGGATGCCGGGGTTGAGTC | |
| | AGGTTCCGTAATCGGACCGGCATGGGACTC | |
| | ACTGCTGGCTAAACTTATCGTTACCGGGCG | |
| | TACACGTGCCGAGGCGCTTCAGCGCGCAG | |
| | CCCGCGCCTTAGATGAATTTACGGTTGAGG | |
| | GCATGGCAACCGCGATCCCTTTCCATCGCA | |
| | CAGTAGTACGCGATCCAGCATTCGCTCCTG | |
| | AGCTTACCGGGTCAACGGACCCATTCACCG | |
| | TTCATACACGCTGGATTGAAACTGAATTTG | |
| | TCAACGAAATTAAGCCTTTTACCACCCCTG | |
| | CCGACACGGAGACAGATGAAGAGTCTGGG | |
| | CGCGAGACAGTGGTAGTCGAGGTCGGTGG | |
| | GAAACGCTTAGAGGTAAGTCTTCCGTCCAG | |
| | CCTGGGAATGTCGTTGGCCCGTACCGGCCT | |
| | TGCCGCGGGGGCCCGCCCCAAACGCCGCG | |
| | CGGCCAAGAAGTCAGGCCCTGCAGCATCG | |
| | GGTGATACACTGGCATCTCCTATGCAAGGT | |
| | ACGATCGTAAAGATCGCCGTGGAAGAGGGA | |
| | CAAGAAGTACAGGAGGGAGATCTGATTGTGG | |
| | TTCTTGAAGCTATGAAGATGGAACAGCCACTT | |
| | AATGCCCACCGTTCGGGAACCATTAAGGGGCT | |
| | TACTGCTGAAGTAGGTGCTTCACTGACGTCGG | |
| | GCGCCGCTATCTGTGAAATCAAGGATTGATAA | |
| | CGCTAACGAAAAAGTTAAATACAGGAACAAG | |
| | AGAACATATGTCGGAGCCCGAGGAACAGCA | |
| | GCCAGATATCCACACGACAGCGGGCAAGTT | |
| | AGCTGATCTTCGTCGCCGCATCGAAGAGGC | |
| | AACGCACGCCGGTTCTGCGCGCGCGGTGG | |
| | AGAAACAGCACGCGAAGGGTAAACTTACG | |
| | GCTCGTGAGCGTATCGATTTGTTGCTGGAC | |
| | GAAGGGTCTTTTGTAGAGCTTGATGAGTTT | |
| | GCGCGTCACCGTTCGACGAATTTCGGACTG | |
| | GATGCCAACCGTCCATATGGAGATGGAGTG | |
| | GTGACTGGCTATGGAACTGTTGACGGACGT | |
| | CCGGTTGCCGTCTTTTCGCAAGACTTTACG | |
| | GTCTTTGGGGGCGCTCTGGGGGAAGTATAC | |
| | GGGCAAAAAATTGTGAAGGTCATGGATTTC | |
| | GCTCTTAAGACCGGGTGTCCCGTCGTGGGT | |
| | ATTAATGACTCAGGTGGGGCACGCATTCAA | |
| | GAGGGTGTAGCAAGTCTGGGCGCGTATGG | |
| | AGAGATTTTCCGTCGCAATACGCACGCGTC | |
| | GGGCGTGATCCCTCAGATTTCGCTTGTAGT | |
| | TGGCCCATGCGCAGGGGGAGCTGTGTACT | |
| | CTCCAGCTATTACTGACTTTACGGTAATGG | |
| | TCGACCAAACATCGCATATGTTTATCACCG | |
| | GACCCGATGTGATTAAGACAGTGACAGGG | |
| | GAGGATGTGGGTTTTGAGGAACTTGGTGGT | |
| | GCGCGTACGCACAACAGTACGTCTGGGGTT | |
| | GCCCATCATATGGCTGGGGATGAGAAAGAC | |
| | GCTGTGGAGTATGTTAAGCAATTATTGAGT | |
| | TATTTGCCGTCGAACAATTTAAGTGAGCCT | |
| | CCGGCGTTTCCTGAAGAGGCTGATTTAGCC | |
| | GTTACGGACGAAGATGCGGAATTAGATACA | |
| | ATTGTGCCGGATTCGGCTAACCAACCCTAT | |
| | GATATGCATTCTGTAATCGAGCATGTCCTT | |
| | GACGATGCGGAATTTTTCGAGACTCAACCG | |
| | TTGTTTGCCCCCAACATCCTGACCGGCTTT | |
| | GGTCGCGTTGAAGGCCGTCCGGTGGGTAT | |
| | CGTGGCGAATCAGCCGATGCAGTTTGCTGG | |
| | ATGCTTAGATATCACTGCCTCAGAAAAAGC | |
| | TGCTCGTTTCGTTCGCACTTGCGACGCTTT | |
| | CAACGTCCCTGTGCTTACGTTTGTAGACGT | |
| | CCCCGGGTTTTTACCGGGCGTAGATCAGGA | |
| | GCATGACGGGATCATCCGCCGCGGTGCGA | |
| | AGTTGATTTTTGCCTATGCAGAAGCGACCG | |
| | TGCCGTTGATCACAGTAATCACGCGCAAAG | |
| | CCTTCGGAGGTGCGTATGACGTAATGGGCT | |
| | CAAAACACCTTGGCGCTGACCTTAATCTGG | |
| | CATGGCCCACGGCCCAAATCGCTGTAATGG | |
| | GCGCTCAAGGTGCTGTAAACATCCTTCATC | |
| | GTCGTACGATTGCAGATGCGGGGGACGAT | |
| | GCGGAAGCCACGCGCGCCCGTTTAATTCAA | |
| | GAGTACGAGGATGCTTTATTAAATCCCTAT | |
| | ACTGCGGCTGAGCGCGGGTATGTAGACGC | |
| | GGTCATCATGCCCTCAGATACTCGCCGTCA | |
| | TATCGTACGTGGTTTACGCCAATTACGCAC | |
| | CAAGCGCGAGTCTTTACCCCCGAAAAAGCA | |
| | CGGGAACATTCCCCTT | |
| Construct comprising a | TAAGGCGTAAGTTCAACAGGAGAGCATTATG | SEQ |
| prpE-accA-pccB gene | TCTTTTAGCGAATTTTATCAGCGTTCGATTA | ID NO: |
| cassette; (as shown in FIG. | ACGAACCGGAGAAGTTCTGGGCCGAGCAG | 37 |
| 15B and FIG. 16) | GCCCGGCGTATTGACTGGCAGACGCCCTTT | |
| ribosome binding sites are | ACGCAAACGCTCGACCACAGCAACCCGCCG | |
| underlined; coding | TTTGCCCGTTGGTTTTGTGAAGGCCGAACC | |
| sequences bold and | AACTTGTGTCACAACGCTATCGACCGCTGG | |
| underlined | CTGGAGAAACAGCCAGAGGCGCTGGCATT | |
| | GATTGCCGTCTCTTCGGAAACAGAGGAAGA | |
| | GCGTACCTTTACCTTCCGCCAGTTACATGA | |
| | CGAAGTGAATGCGGTGGCGTCAATGCTGC | |
| | GCTCACTGGGCGTGCAGCGTGGCGATCGG | |
| | GTGCTGGTGTATATGCCGATGATTGCCGAA | |
| | GCGCATATTACCCTGCTGGCCTGCGCGCGC | |
| | ATTGGTGCTATTCACTCGGTGGTGTTTGGG | |
| | GGATTTGCTTCGCACAGCGTGGCAACGCGA | |
| | ATTGATGACGCTAAACCGGTGCTGATTGTC | |
| | TCGGCTGATGCCGGGGCGCGCGGCGGTAA | |
| | AATCATTCCGTATAAAAAATTGCTCGACGA | |
| | TGCGATAAGTCAGGCACAGCATCAGCCGCG | |
| | TCACGTTTTACTGGTGGATCGCGGGCTGGC | |
| | GAAAATGGCGCGCGTTAGCGGGCGGGATG | |
| | TCGATTTCGCGTCGTTGCGCCATCAACACA | |
| | TCGGCGCGCGGGTGCCGGTGGCATGGCTG | |
| | GAATCCAACGAAACCTCCTGCATTCTCTAC | |
| | ACCTCCGGCACGACCGGCAAACCTAAAGGT | |
| | GTGCAGCGTGATGTCGGCGGATATGCGGT | |
| | GGCGCTGGCGACCTCGATGGACACCATTTT | |
| | TGGCGGCAAAGCGGGCGGCGTGTTCTTTTG | |
| | TGCTTCGGATATCGGCTGGGTGGTAGGGCA | |
| | TTCGTATATCGTTTACGCGCCGCTGCTGGC | |
| | GGGGATGGCGACTATCGTTTACGAAGGATT | |
| | GCCGACCTGGCCGGACTGCGGCGTGTGGT | |
| | GGAAAATTGTCGAGAAATATCAGGTTAGCC | |
| | GCATGTTCTCAGCGCCGACCGCCATTCGCG | |
| | TGCTGAAAAAATTCCCTACCGCTGAAATTC | |
| | GCAAACACGATCTTTCGTCGCTGGAAGTGC | |
| | TCTATCTGGCTGGAGAACCGCTGGACGAGC | |
| | CGACCGCCAGTTGGGTGAGCAATACGCTG | |
| | GATGTGCCGGTCATCGACAACTACTGGCAG | |
| | ACCGAATCCGGCTGGCCGATTATGGCGATT | |
| | GCTCGCGGTCTGGATGACAGACCGACGCG | |
| | TCTGGGAAGCCCCGGCGTGCCGATGTATG | |
| | GCTATAACGTGCAGTTGCTCAATGAAGTCA | |
| | CCGGCGAACCGTGTGGCGTCAATGAGAAA | |
| | GGGATGCTGGTAGTGGAGGGGCCATTGCC | |
| | GCCAGGCTGTATTCAAACCATCTGGGGCGA | |
| | CGACGACCGCTTTGTGAAGACGTACTGGTC | |
| | GCTGTTTTCCCGTCCGGTGTACGCCACTTT | |
| | TGACTGGGGCATCCGCGATGCTGACGGTTA | |
| | TCACTTTATTCTCGGGCGCACTGACGATGT | |
| | GATTAACGTTGCCGGACATCGGCTGGGTAC | |
| | GCGTGAGATTGAAGAGAGTATCTCCAGTCA | |
| | TCCGGGCGTTGCCGAAGTGGCGGTGGTTG | |
| | GGGTGAAAGATGCGCTGAAAGGGCAGGTG | |
| | GCGGTGGCGTTTGTCATTCCGAAAGAGAGC | |
| | GACAGTCTGGAAGACCGTGAGGTGGCGCA | |
| | CTCGCAAGAGAAGGCGATTATGGCGCTGGT | |
| | GGACAGCCAGATTGGCAACTTTGGCCGCCC | |
| | GGCGCACGTCTGGTTTGTCTCGCAATTGCC | |
| | AAAAACGCGATCCGGAAAAATGCTGCGCCG | |
| | CACGATCCAGGCGATTTGCGAAGGACGCG | |
| | ATCCTGGGGATCTGACGACCATTGATGATC | |
| | CGGCGTCGTTGGATCAGATCCGCCAGGCG | |
| | ATGGAAGAGTAGTACTAGATTCAATATAGAG | |
| | TAAAAGAGGTAAGAGTATCCATGCGTAAAGT | |
| | TCTGATCGCTAATCGTGGAGAAATTGCTGT | |
| | ACGTGTAGCACGTGCATGTCGTGATGCGGG | |
| | AATCGCATCAGTAGCCGTATACGCGGACCC | |
| | GGATCGTGACGCGTTGCATGTGCGCGCGG | |
| | CGGACGAAGCATTTGCACTGGGTGGTGATA | |
| | CGCCTGCAACATCTTACTTAGACATCGCCA | |
| | AGGTGTTAAAGGCTGCACGTGAGAGTGGT | |
| | GCAGACGCCATTCATCCCGGTTACGGCTTT | |
| | TTAAGTGAAAATGCCGAGTTCGCGCAGGCC | |
| | GTGTTAGATGCGGGTCTTATCTGGATCGGA | |
| | CCACCGCCCCATGCAATCCGCGATCGTGGG | |
| | GAAAAAGTTGCAGCTCGCCATATTGCCCAG | |
| | CGTGCTGGGGCGCCGCTGGTTGCGGGCAC | |
| | CCCTGACCCGGTTTCTGGTGCTGACGAAGT | |
| | CGTCGCCTTCGCGAAAGAGCATGGACTGCC | |
| | GATCGCGATTAAGGCTGCTTTTGGAGGCGG | |
| | TGGTCGTGGTTTAAAGGTTGCCCGTACATT | |
| | GGAAGAAGTGCCCGAGTTATATGACTCCGC | |
| | CGTGCGTGAAGCTGTGGCGGCATTCGGAC | |
| | GTGGCGAATGTTTCGTGGAGCGCTATTTAG | |
| | ACAAACCGCGTCATGTAGAAACCCAGTGCT | |
| | TGGCAGATACTCACGGTAATGTAGTTGTGG | |
| | TTTCTACTCGCGACTGTTCGTTACAGCGTC | |
| | GTCATCAGAAACTGGTAGAGGAGGCACCC | |
| | GCCCCGTTTTTAAGCGAAGCTCAGACAGAG | |
| | CAACTGTACTCCTCCTCCAAGGCTATTCTT | |
| | AAGGAAGCTGGGTATGGTGGAGCGGGAAC | |
| | CGTTGAGTTTTTAGTAGGTATGGATGGTAC | |
| | TATCTTCTTCTTGGAGGTCAATACCCGCCT | |
| | GCAGGTGGAGCACCCTGTGACCGAAGAAG | |
| | TCGCAGGGATCGACCTGGTCCGTGAAATGT | |
| | TCCGCATTGCAGATGGCGAGGAGCTGGGG | |
| | TACGACGATCCAGCCCTTCGCGGCCACTCG | |
| | TTCGAATTTCGCATCAATGGGGAGGACCCA | |
| | GGTCGTGGTTTTTTGCCCGCACCTGGTACG | |
| | GTTACGCTTTTTGATGCTCCGACCGGACCC | |
| | GGAGTCCGCCTGGATGCCGGGGTTGAGTC | |
| | AGGTTCCGTAATCGGACCGGCATGGGACTC | |
| | ACTGCTGGCTAAACTTATCGTTACCGGGCG | |
| | TACACGTGCCGAGGCGCTTCAGCGCGCAG | |
| | CCCGCGCCTTAGATGAATTTACGGTTGAGG | |
| | GCATGGCAACCGCGATCCCTTTCCATCGCA | |
| | CAGTAGTACGCGATCCAGCATTCGCTCCTG | |
| | AGCTTACCGGGTCAACGGACCCATTCACCG | |
| | TTCATACACGCTGGATTGAAACTGAATTTG | |
| | TCAACGAAATTAAGCCTTTTACCACCCCTG | |
| | CCGACACGGAGACAGATGAAGAGTCTGGG | |
| | CGCGAGACAGTGGTAGTCGAGGTCGGTGG | |
| | GAAACGCTTAGAGGTAAGTCTTCCGTCCAG | |
| | CCTGGGAATGTCGTTGGCCCGTACCGGCCT | |
| | TGCCGCGGGGGCCCGCCCCAAACGCCGCG | |
| | CGGCCAAGAAGTCAGGCCCTGCAGCATCG | |
| | GGTGATACACTGGCATCTCCTATGCAAGGT | |
| | ACGATCGTAAAGATCGCCGTGGA | |
| | AGAGGGACAAGAAGTACAGGAGGGAGATCTG | |
| | ATTGTGGTTCTTGAAGCTATGAAGATGGAACA | |
| | GCCACTTAATGCCCACCGTTCGGGAACCATTA | |
| | AGGGGCTTACTGCTGAAGTAGGTGCTTCACTG | |
| | ACGTCGGGCGCCGCTATCTGTGAAATCAAGG | |
| | ATTGATAACGCTAACGAAAAAGTTAAATACA | |
| | GGAACAAGAGAACATATGTCGGAGCCCGAG | |
| | GAACAGCAGCCAGATATCCACACGACAGCG | |
| | GGCAAGTTAGCTGATCTTCGTCGCCGCATC | |
| | GAAGAGGCAACGCACGCCGGTTCTGCGCG | |
| | CGCGGTGGAGAAACAGCACGCGAAGGGTA | |
| | AACTTACGGCTCGTGAGCGTATCGATTTGT | |
| | TGCTGGACGAAGGGTCTTTTGTAGAGCTTG | |
| | ATGAGTTTGCGCGTCACCGTTCGACGAATT | |
| | TCGGACTGGATGCCAACCGTCCATATGGAG | |
| | ATGGAGTGGTGACTGGCTATGGAACTGTTG | |
| | ACGGACGTCCGGTTGCCGTCTTTTCGCAAG | |
| | ACTTTACGGTCTTTGGGGGCGCTCTGGGGG | |
| | AAGTATACGGGCAAAAAATTGTGAAGGTCA | |
| | TGGATTTCGCTCTTAAGACCGGGTGTCCCG | |
| | TCGTGGGTATTAATGACTCAGGTGGGGCAC | |
| | GCATTCAAGAGGGTGTAGCAAGTCTGGGC | |
| | GCGTATGGAGAGATTTTCCGTCGCAATACG | |
| | CACGCGTCGGGCGTGATCCCTCAGATTTCG | |
| | CTTGTAGTTGGCCCATGCGCAGGGGGAGCT | |
| | GTGTACTCTCCAGCTATTACTGACTTTACG | |
| | GTAATGGTCGACCAAACATCGCATATGTTT | |
| | ATCACCGGACCCGATGTGATTAAGACAGTG | |
| | ACAGGGGAGGATGTGGGTTTTGAGGAACTT | |
| | GGTGGTGCGCGTACGCACAACAGTACGTCT | |
| | GGGGTTGCCCATCATATGGCTGGGGATGA | |
| | GAAAGACGCTGTGGAGTATGTTAAGCAATT | |
| | ATTGAGTTATTTGCCGTCGAACAATTTAAG | |
| | TGAGCCTCCGGCGTTTCCTGAAGAGGCTGA | |
| | TTTAGCCGTTACGGACGAAGATGCGGAATT | |
| | AGATACAATTGTGCCGGATTCGGCTAACCA | |
| | ACCCTATGATATGCATTCTGTAATCGAGCA | |
| | TGTCCTTGACGATGCGGAATTTTTCGAGAC | |
| | TCAACCGTTGTTTGCCCCCAACATCCTGAC | |
| | CGGCTTTGGTCGCGTTGAAGGCCGTCCGGT | |
| | GGGTATCGTGGCGAATCAGCCGATGCAGTT | |
| | TGCTGGATGCTTAGATATCACTGCCTCAGA | |
| | AAAAGCTGCTCGTTTCGTTCGCACTTGCGA | |
| | CGCTTTCAACGTCCCTGTGCTTACGTTTGT | |
| | AGACGTCCCCGGGTTTTTACCGGGCGTAGA | |
| | TCAGGAGCATGACGGGATCATCCGCCGCG | |
| | GTGCGAAGTTGATTTTTGCCTATGCAGAAG | |
| | CGACCGTGCCGTTGATCACAGTAATCACGC | |
| | GCAAAGCCTTCGGAGGTGCGTATGACGTAA | |
| | TGGGCTCAAAACACCTTGGCGCTGACCTTA | |
| | ATCTGGCATGGCCCACGGCCCAAATCGCTG | |
| | TAATGGGCGCTCAAGGTGCTGTAAACATCC | |
| | TTCATCGTCGTACGATTGCAGATGCGGGGG | |
| | ACGATGCGGAAGCCACGCGCGCCCGTTTAA | |
| | TTCAAGAGTACGAGGATGCTTTATTAAATC | |
| | CCTATACTGCGGCTGAGCGCGGGTATGTAG | |
| | ACGCGGTCATCATGCCCTCAGATACTCGCC | |
| | GTCATATCGTACGTGGTTTACGCCAATTAC | |
| | GCACCAAGCGCGAGTCTTTACCCCCGAAAA | |
| | AGCACGGGAACATTCCCCTT | |
| prpE sequence (comprised | ATGTCTTTTAGCGAATTTTATCAGCGTTCGATT | SEQ |
| in the prpE-accA-pccB | AACGAACCGGAGAAGTTCTGGGCCGAGCAGG | ID NO: |
| construct shown in FIG. | CCCGGCGTATTGACTGGCAGACGCCCTTTACG | 25 |
| 15B and FIG. 16) | CAAACGCTCGACCACAGCAACCCGCCGTTTGC | |
| | CCGTTGGTTTTGTGAAGGCCGAACCAACTTGT | |
| | GTCACAACGCTATCGACCGCTGGCTGGAGAA | |
| | ACAGCCAGAGGCGCTGGCATTGATTGCCGTCT | |
| | CTTCGGAAACAGAGGAAGAGCGTACCTTTAC | |
| | CTTCCGCCAGTTACATGACGAAGTGAATGCGG | |
| | TGGCGTCAATGCTGCGCTCACTGGGCGTGCAG | |
| | CGTGGCGATCGGGTGCTGGTGTATATGCCGAT | |
| | GATTGCCGAAGCGCATATTACCCTGCTGGCCT | |
| | GCGCGCGCATTGGTGCTATTCACTCGGTGGTG | |
| | TTTGGGGGATTTGCTTCGCACAGCGTGGCAAC | |
| | GCGAATTGATGACGCTAAACCGGTGCTGATTG | |
| | TCTCGGCTGATGCCGGGGCGCGCGGCGGTAA | |
| | AATCATTCCGTATAAAAAATTGCTCGACGATG | |
| | CGATAAGTCAGGCACAGCATCAGCCGCGTCA | |
| | CGTTTTACTGGTGGATCGCGGGCTGGCGAAAA | |
| | TGGCGCGCGTTAGCGGGCGGGATGTCGATTTC | |
| | GCGTCGTTGCGCCATCAACACATCGGCGCGCG | |
| | GGTGCCGGTGGCATGGCTGGAATCCAACGAA | |
| | ACCTCCTGCATTCTCTACACCTCCGGCACGAC | |
| | CGGCAAACCTAAAGGTGTGCAGCGTGATGTC | |
| | GGCGGATATGCGGTGGCGCTGGCGACCTCGA | |
| | TGGACACCATTTTTGGCGGCAAAGCGGGCGG | |
| | CGTGTTCTTTTGTGCTTCGGATATCGGCTGGGT | |
| | GGTAGGGCATTCGTATATCGTTTACGCGCCGC | |
| | TGCTGGCGGGGATGGCGACTATCGTTTACGAA | |
| | GGATTGCCGACCTGGCCGGACTGCGGCGTGTG | |
| | GTGGAAAATTGTCGAGAAATATCAGGTTAGC | |
| | CGCATGTTCTCAGCGCCGACCGCCATTCGCGT | |
| | GCTGAAAAAATTCCCTACCGCTGAAATTCGCA | |
| | AACACGATCTTTCGTCGCTGGAAGTGCTCTAT | |
| | CTGGCTGGAGAACCGCTGGACGAGCCGACCG | |
| | CCAGTTGGGTGAGCAATACGCTGGATGTGCCG | |
| | GTCATCGACAACTACTGGCAGACCGAATCCG | |
| | GCTGGCCGATTATGGCGATTGCTCGCGGTCTG | |
| | GATGACAGACCGACGCGTCTGGGAAGCCCCG | |
| | GCGTGCCGATGTATGGCTATAACGTGCAGTTG | |
| | CTCAATGAAGTCACCGGCGAACCGTGTGGCGT | |
| | CAATGAGAAAGGGATGCTGGTAGTGGAGGGG | |
| | CCATTGCCGCCAGGCTGTATTCAAACCATCTG | |
| | GGGCGACGACGACCGCTTTGTGAAGACGTAC | |
| | TGGTCGCTGTTTTCCCGTCCGGTGTACGCCAC | |
| | TTTTGACTGGGGCATCCGCGATGCTGACGGTT | |
| | ATCACTTTATTCTCGGGCGCACTGACGATGTG | |
| | ATTAACGTTGCCGGACATCGGCTGGGTACGCG | |
| | TGAGATTGAAGAGAGTATCTCCAGTCATCCGG | |
| | GCGTTGCCGAAGTGGCGGTGGTTGGGGTGAA | |
| | AGATGCGCTGAAAGGGCAGGTGGCGGTGGCG | |
| | TTTGTCATTCCGAAAGAGAGCGACAGTCTGGA | |
| | AGACCGTGAGGTGGCGCACTCGCAAGAGAAG | |
| | GCGATTATGGCGCTGGTGGACAGCCAGATTG | |
| | GCAACTTTGGCCGCCCGGCGCACGTCTGGTTT | |
| | GTCTCGCAATTGCCAAAAACGCGATCCGGAA | |
| | AAATGCTGCGCCGCACGATCCAGGCGATTTGC | |
| | GAAGGACGCGATCCTGGGGATCTGACGACCA | |
| | TTGATGATCCGGCGTCGTTGGATCAGATCCGC | |
| | CAGGCGATGGAAGAGTAG | |
| accA sequence (comprised | ATGCGTAAAGTTCTGATCGCTAATCGTGGAGA | SEQ |
| in the prpE-accA-pccB | AATTGCTGTACGTGTAGCACGTGCATGTCGTG | ID NO: |
| construct shown in FIG. | ATGCGGGAATCGCATCAGTAGCCGTATACGC | 38 |
| 15B and FIG. 16) | GGACCCGGATCGTGACGCGTTGCATGTGCGCG | |
| | CGGCGGACGAAGCATTTGCACTGGGTGGTGA | |
| | TACGCCTGCAACATCTTACTTAGACATCGCCA | |
| | AGGTGTTAAAGGCTGCACGTGAGAGTGGTGC | |
| | AGACGCCATTCATCCCGGTTACGGCTTTTTAA | |
| | GTGAAAATGCCGAGTTCGCGCAGGCCGTGTTA | |
| | GATGCGGGTCTTATCTGGATCGGACCACCGCC | |
| | CCATGCAATCCGCGATCGTGGGGAAAAAGTT | |
| | GCAGCTCGCCATATTGCCCAGCGTGCTGGGGC | |
| | GCCGCTGGTTGCGGGCACCCCTGACCCGGTTT | |
| | CTGGTGCTGACGAAGTCGTCGCCTTCGCGAAA | |
| | GAGCATGGACTGCCGATCGCGATTAAGGCTG | |
| | CTTTTGGAGGCGGTGGTCGTGGTTTAAAGGTT | |
| | GCCCGTACATTGGAAGAAGTGCCCGAGTTATA | |
| | TGACTCCGCCGTGCGTGAAGCTGTGGCGGCAT | |
| | TCGGACGTGGCGAATGTTTCGTGGAGCGCTAT | |
| | TTAGACAAACCGCGTCATGTAGAAACCCAGT | |
| | GCTTGGCAGATACTCACGGTAATGTAGTTGTG | |
| | GTTTCTACTCGCGACTGTTCGTTACAGCGTCG | |
| | TCATCAGAAACTGGTAGAGGAGGCACCCGCC | |
| | CCGTTTTTAAGCGAAGCTCAGACAGAGCAACT | |
| | GTACTCCTCCTCCAAGGCTATTCTTAAGGAAG | |
| | CTGGGTATGGTGGAGCGGGAACCGTTGAGTTT | |
| | TTAGTAGGTATGGATGGTACTATCTTCTTCTTG | |
| | GAGGTCAATACCCGCCTGCAGGTGGAGCACC | |
| | CTGTGACCGAAGAAGTCGCAGGGATCGACCT | |
| | GGTCCGTGAAATGTTCCGCATTGCAGATGGCG | |
| | AGGAGCTGGGGTACGACGATCCAGCCCTTCG | |
| | CGGCCACTCGTTCGAATTTCGCATCAATGGGG | |
| | AGGACCCAGGTCGTGGTTTTTTGCCCGCACCT | |
| | GGTACGGTTACGCTTTTTGATGCTCCGACCGG | |
| | ACCCGGAGTCCGCCTGGATGCCGGGGTTGAGT | |
| | CAGGTTCCGTAATCGGACCGGCATGGGACTCA | |
| | CTGCTGGCTAAACTTATCGTTACCGGGCGTAC | |
| | ACGTGCCGAGGCGCTTCAGCGCGCAGCCCGC | |
| | GCCTTAGATGAATTTACGGTTGAGGGCATGGC | |
| | AACCGCGATCCCTTTCCATCGCACAGTAGTAC | |
| | GCGATCCAGCATTCGCTCCTGAGCTTACCGGG | |
| | TCAACGGACCCATTCACCGTTCATACACGCTG | |
| | GATTGAAACTGAATTTGTCAACGAAATTAAGC | |
| | CTTTTACCACCCCTGCCGACACGGAGACAGAT | |
| | GAAGAGTCTGGGCGCGAGACAGTGGTAGTCG | |
| | AGGTCGGTGGGAAACGCTTAGAGGTAAGTCT | |
| | TCCGTCCAGCCTGGGAATGTCGTTGGCCCGTA | |
| | CCGGCCTTGCCGCGGGGGCCCGCCCCAAACG | |
| | CCGCGCGGCCAAGAAGTCAGGCCCTGCAGCA | |
| | TCGGGTGATACACTGGCATCTCCTATGCAAGG | |
| | TACGATCGTAAAGATCGCCGTGGAAGAGGGA | |
| | CAAGAAGTACAGGAGGGAGATCTGATTGTGG | |
| | TTCTTGAAGCTATGAAGATGGAACAGCCACTT | |
| | AATGCCCACCGTTCGGGAACCATTAAGGGGCT | |
| | TACTGCTGAAGTAGGTGCTTCACTGACGTCGG | |
| | GCGCCGCTATCTGTGAAATCAAGGATTG | |
| pccB sequence (comprised | ATGTCGGAGCCCGAGGAACAGCAGCCAGATA | SEQ |
| in the prpE-accA-pccB | TCCACACGACAGCGGGCAAGTTAGCTGATCTT | ID NO: |
| construct shown in FIG. | CGTCGCCGCATCGAAGAGGCAACGCACGCCG | 39 |
| 15B and FIG. 16) | GTTCTGCGCGCGCGGTGGAGAAACAGCACGC | |
| | GAAGGGTAAACTTACGGCTCGTGAGCGTATC | |
| | GATTTGTTGCTGGACGAAGGGTCTTTTGTAGA | |
| | GCTTGATGAGTTTGCGCGTCACCGTTCGACGA | |
| | ATTTCGGACTGGATGCCAACCGTCCATATGGA | |
| | GATGGAGTGGTGACTGGCTATGGAACTGTTGA | |
| | CGGACGTCCGGTTGCCGTCTTTTCGCAAGACT | |
| | TTACGGTCTTTGGGGGCGCTCTGGGGGAAGTA | |
| | TACGGGCAAAAAATTGTGAAGGTCATGGATTT | |
| | CGCTCTTAAGACCGGGTGTCCCGTCGTGGGTA | |
| | TTAATGACTCAGGTGGGGCACGCATTCAAGA | |
| | GGGTGTAGCAAGTCTGGGCGCGTATGGAGAG | |
| | ATTTTCCGTCGCAATACGCACGCGTCGGGCGT | |
| | GATCCCTCAGATTTCGCTTGTAGTTGGCCCAT | |
| | GCGCAGGGGGAGCTGTGTACTCTCCAGCTATT | |
| | ACTGACTTTACGGTAATGGTCGACCAAACATC | |
| | GCATATGTTTATCACCGGACCCGATGTGATTA | |
| | AGACAGTGACAGGGGAGGATGTGGGTTTTGA | |
| | GGAACTTGGTGGTGCGCGTACGCACAACAGT | |
| | ACGTCTGGGGTTGCCCATCATATGGCTGGGGA | |
| | TGAGAAAGACGCTGTGGAGTATGTTAAGCAA | |
| | TTATTGAGTTATTTGCCGTCGAACAATTTAAG | |
| | TGAGCCTCCGGCGTTTCCTGAAGAGGCTGATT | |
| | TAGCCGTTACGGACGAAGATGCGGAATTAGA | |
| | TACAATTGTGCCGGATTCGGCTAACCAACCCT | |
| | ATGATATGCATTCTGTAATCGAGCATGTCCTT | |
| | GACGATGCGGAATTTTTCGAGACTCAACCGTT | |
| | GTTTGCCCCCAACATCCTGACCGGCTTTGGTC | |
| | GCGTTGAAGGCCGTCCGGTGGGTATCGTGGCG | |
| | AATCAGCCGATGCAGTTTGCTGGATGCTTAGA | |
| | TATCACTGCCTCAGAAAAAGCTGCTCGTTTCG | |
| | TTCGCACTTGCGACGCTTTCAACGTCCCTGTG | |
| | CTTACGTTTGTAGACGTCCCCGGGTTTTTACC | |
| | GGGCGTAGATCAGGAGCATGACGGGATCATC | |
| | CGCCGCGGTGCGAAGTTGATTTTTGCCTATGC | |
| | AGAAGCGACCGTGCCGTTGATCACAGTAATC | |
| | ACGCGCAAAGCCTTCGGAGGTGCGTATGACG | |
| | TAATGGGCTCAAAACACCTTGGCGCTGACCTT | |
| | AATCTGGCATGGCCCACGGCCCAAATCGCTGT | |
| | AATGGGCGCTCAAGGTGCTGTAAACATCCTTC | |
| | ATCGTCGTACGATTGCAGATGCGGGGGACGA | |
| | TGCGGAAGCCACGCGCGCCCGTTTAATTCAAG | |
| | AGTACGAGGATGCTTTATTAAATCCCTATACT | |
| | GCGGCTGAGCGCGGGTATGTAGACGCGGTCA | |
| | TCATGCCCTCAGATACTCGCCGTCATATCGTA | |
| | CGTGGTTTACGCCAATTACGCACCAAGCGCGA | |
| | GTCTTTACCCCCGAAAAAGCACGGGAACATTC | |
| | CCCTTTG | |

In certain constructs, the prpE pccB, -accA1 and mmcE-mutAB cassettes are operably linked to a FNR-responsive promoter, which may be is further fused to a strong ribosome binding site sequence. For efficient translation, a 15 base pair ribosome binding site was designed for each synthetic gene in the operon. Each gene cassette and regulatory region construct is expressed on a high-copy plasmid, a low-copy plasmid, or a chromosome.

In certain embodiments the construct is inserted into the bacterial genome at one or more of the following insertion sites in E. coli Nissle: malE/K, araC/BAD, lacZ, thyA, malP/T. Any suitable insertion site may be used (see, e.g., FIG. 32). The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon. At the site of insertion, DNA primers that are homologous to the site of insertion and to the propionate construct are designed. A linear DNA fragment containing the construct with homology to the target site is generated by PCR, and lambda red recombination is performed as described below. The resulting E. coli Nissle bacteria are genetically engineered to express a propionate biosynthesis cassette and produce propionate.

### Example 4. Generation of Engineered Bacteria Comprising a Transporter of Propionate and/or a Propionate Catabolism Enzyme

The pTet-prpE-PhaBCA plasmids (and other plasmids described herein) are transformed into E. coli Nissle, DH5α, or PIR1. All tubes, solutions, and cuvettes are pre-chilled to 4° C. An overnight culture of E. coli (Nissle, DH5α or PIR1) is diluted 1:100 in 4 mL of LB and grown until it reaches an OD600 of 0.4-0.6. 1 mL of the culture is then centrifuged at 13,000 rpm for 1 min in a 1.5 mL microcentrifuge tube and the supernatant is removed. The cells are then washed three times in pre-chilled 10% glycerol and resuspended in 40 uL pre-chilled 10% glycerol. The electroporator is set to 1.8 kV. 1 uL of a pTet-prpE-PhaBCA miniprep is added to the cells, mixed by pipetting, and pipetted into a sterile, chilled 1 mm cuvette. The dry cuvette is placed into the sample chamber, and the electric pulse is applied. 500 uL of room-temperature SOC media is immediately added, and the mixture is transferred to a culture tube and incubated at 37° C. for 1 hr. The cells are spread out on an LB plate containing 50 ug/mL Kanamycin for pTet-prpBCDE and pTet-mctC.

In alternate embodiments, the pTet-prpE-PhaBCA cassettes or Pfnr-prpE-PhaBCA cassettes may be inserted into the Nissle genome through homologous recombination (Genewiz, Cambridge, Mass.).

To create a vector capable of integrating the synthesized the pTet-prpE-PhaBCA or Pfnr-prpE-PhaBCA cassettes into the chromosome, Gibson assembly is first used to add 1000 bp sequences of DNA homologous to the a Nissle e.g., the lacZ locus into the R6K origin plasmid pKD3. This targets DNA cloned between these homology arms to be integrated into the locus, e.g., the lacZ locus in the Nissle genome. Gibson assembly is used to clone the fragment between these arms. PCR was used to amplify the region from this plasmid containing the entire sequence of the homology arms, as well as the prpE-PhaBCA cassettes between them. This PCR fragment is used to transform electrocompetent Nissle-pKD46, a strain that contains a temperature-sensitive plasmid encoding the lambda red recombinase genes. After transformation, cells are grown out for 2 hours before plating on chloramphenicol at 20 ug/mL at 37 degrees C. Growth at 37 degrees C. also cures the pKD46 plasmid. Transformants containing cassette were chloramphenicol resistant and lac-minus

### Example 5. Lambda Red Recombination

Lambda red recombination is used to make chromosomal modifications, e.g., to express one or more prpE-PhaBCA cassette(s) (or other cassettes described herein) in E. coli Nissle. Lambda red is a procedure using recombination enzymes from a bacteriophage lambda to insert a piece of custom DNA into the chromosome of E. coli. A pKD46 plasmid is transformed into the E. coli Nissle host strain. E. coli Nissle cells are grown overnight in LB media. The overnight culture is diluted 1:100 in 5 mL of LB media and grown until it reaches an OD600 of 0.4-0.6. All tubes, solutions, and cuvettes are pre-chilled to 4° C. The E. coli cells are centrifuged at 2,000 rpm for 5 min at 4° C., the supernatant is removed, and the cells are resuspended in 1 mL of 4° C. water. The E. coli are centrifuged at 2,000 rpm for 5 min at 4° C., the supernatant is removed, and the cells are resuspended in 0.5 mL of 4° C. water. The E. coli are centrifuged at 2,000 rpm for 5 min at 4° C., the supernatant is removed, and the cells are resuspended in 0.1 mL of 4° C. water. The electroporator is set to 2.5 kV. 1 ng of pKD46 plasmid DNA is added to the E. coli cells, mixed by pipetting, and pipetted into a sterile, chilled cuvette. The dry cuvette is placed into the sample chamber, and the electric pulse is applied. 1 mL of room-temperature SOC media is immediately added, and the mixture is transferred to a culture tube and incubated at 30° C. for 1 hr. The cells are spread out on a selective media plate and incubated overnight at 30° C.

DNA sequences comprising the desired prpE-PhaBCA cassette(s) shown above are ordered from a gene synthesis company. The lambda enzymes are used to insert this construct into the genome of E. coli Nissle through homologous recombination. The construct is inserted into a specific site in the genome of E. coli Nissle based on its DNA sequence. To insert the construct into a specific site, the homologous DNA sequence flanking the construct is identified, and includes approximately 50 bases on either side of the sequence. The homologous sequences are ordered as part of the synthesized gene. Alternatively, the homologous sequences may be added by PCR. The construct includes an antibiotic resistance marker that may be removed by recombination. The resulting construct comprises approximately 50 bases of homology upstream, a kanamycin resistance marker that can be removed by recombination, the prpE-PhaBCA cassette(s), and approximately 50 bases of homology downstream.

### Example 6. Establishment of Propionic Acidemia Biomarkers in the PCCAA138T Hypomorph Mouse Model

For in vivo studies, PCCAA138T hypomorph mice were obtained for use as a model for propionic acidemia. First, biomarkers for propionic acidemia were established.

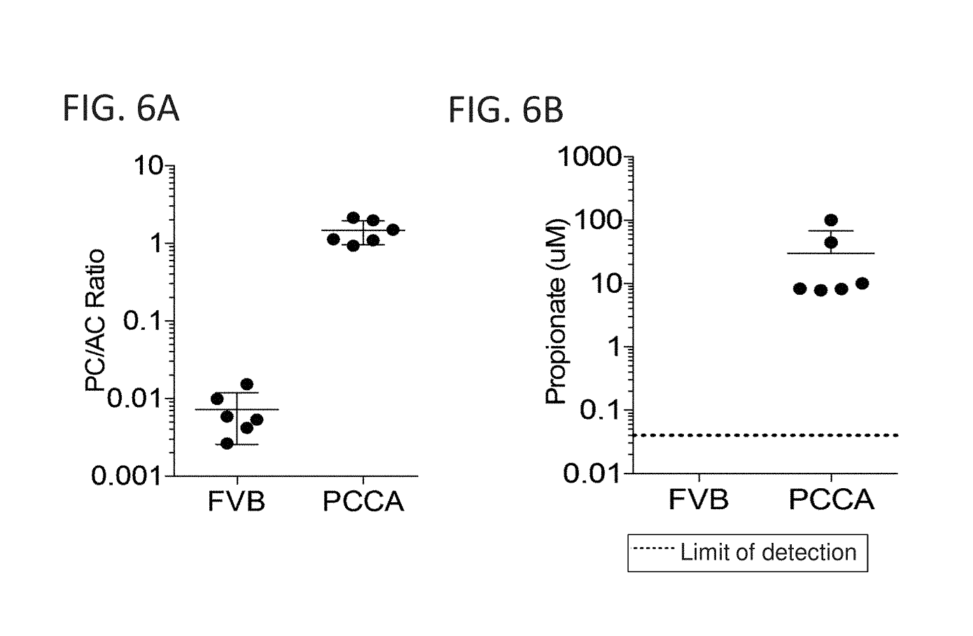

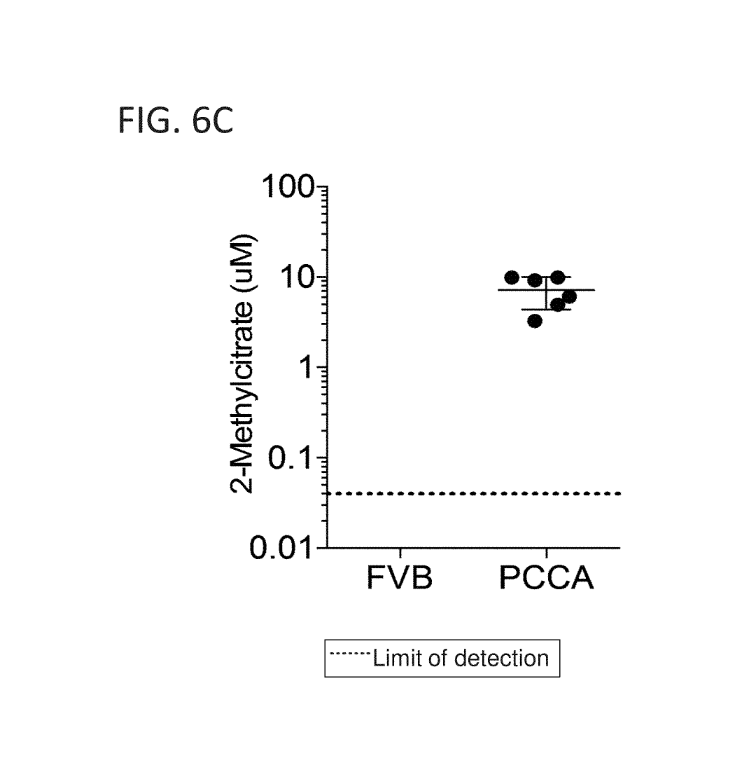

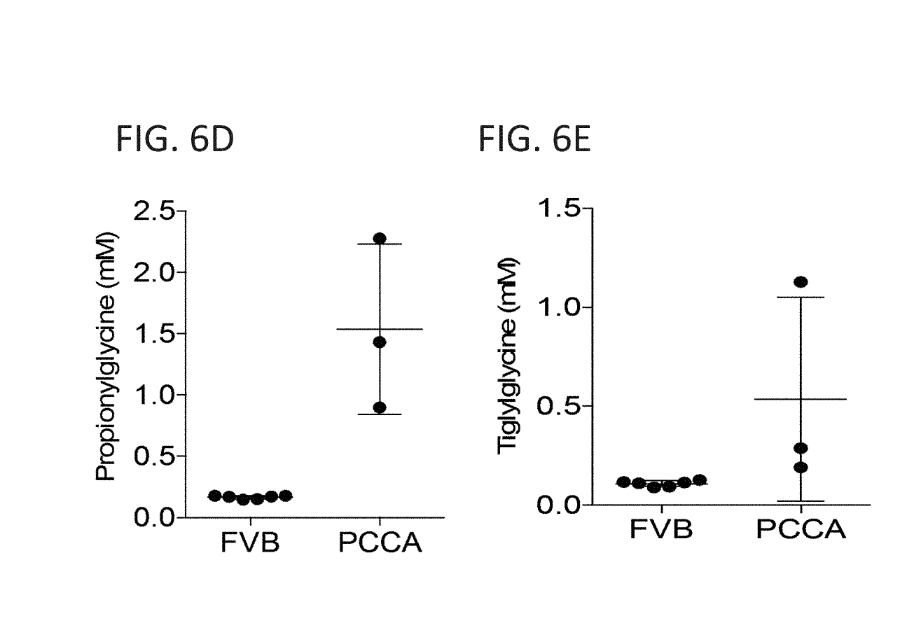

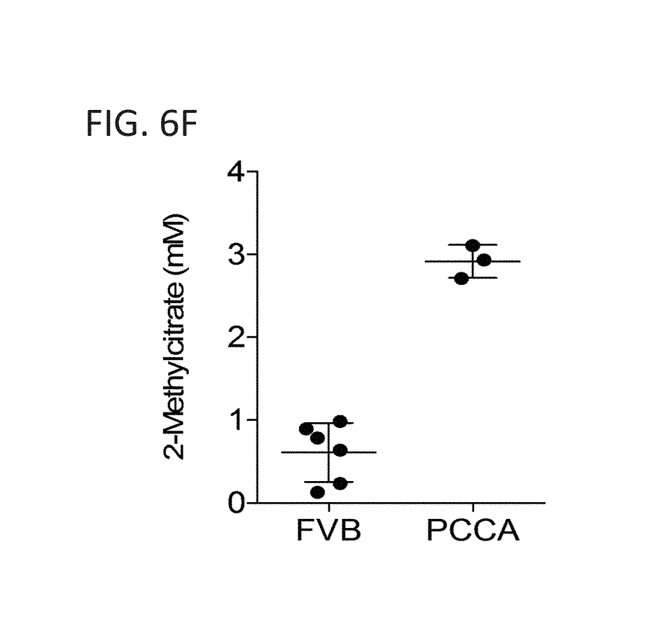

PCCAA138T mice and FVB (parental) controls (10-12 weeks old) were kept on normal chow. Blood and urine were collected and were assayed for known biomarkers of propionic acidemia. In blood, the propionylcarnitine/acetylcarnitine ratio, propionate concentration, and 2-methylcitrate concentration were determined by mass spectrometry as described herein. Results are shown in FIG. 6A-FIG. 6C. For urine, propionyl-glycine, Tigylglycine, and 2-methylcitrate were measured by LC-MS/MS as described herein, and results are shown in FIG. 6D-FIG. 6F.

### Example 7. Enterorecirculation of Propionic Acid in the PCCAA138T Hypomorph Mouse Model

To determine whether propionate undergoes enterorecirculation, in a similar manner as has been hypothesized and shown for amino acids (see e.g., Chang et al., A new theory of enterorecirculation of amino acids and its use for depleting unwanted amino acids using oral enzyme-artificial cells, as in removing phenylalanine in phenylketonuria; Artif Cells Blood Substit Immobil Biotechnol. 1995; 23(1):1-21), levels of enteroconversion of labeled propionate from the bloodstream were measured in various compartments of the gut using the PCCAA138T mouse model.

All PCCAA138T mice (10-12 weeks old) were kept on normal chow until 0.1 mg/g isotopic propionic acid was administered at T0 by subcutaneous injection.

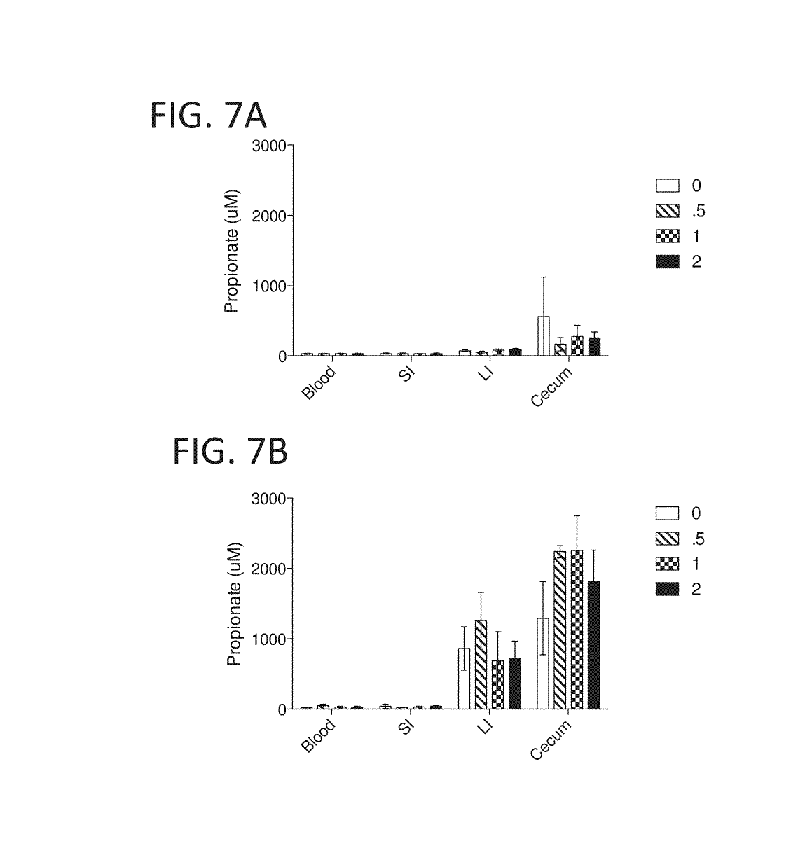

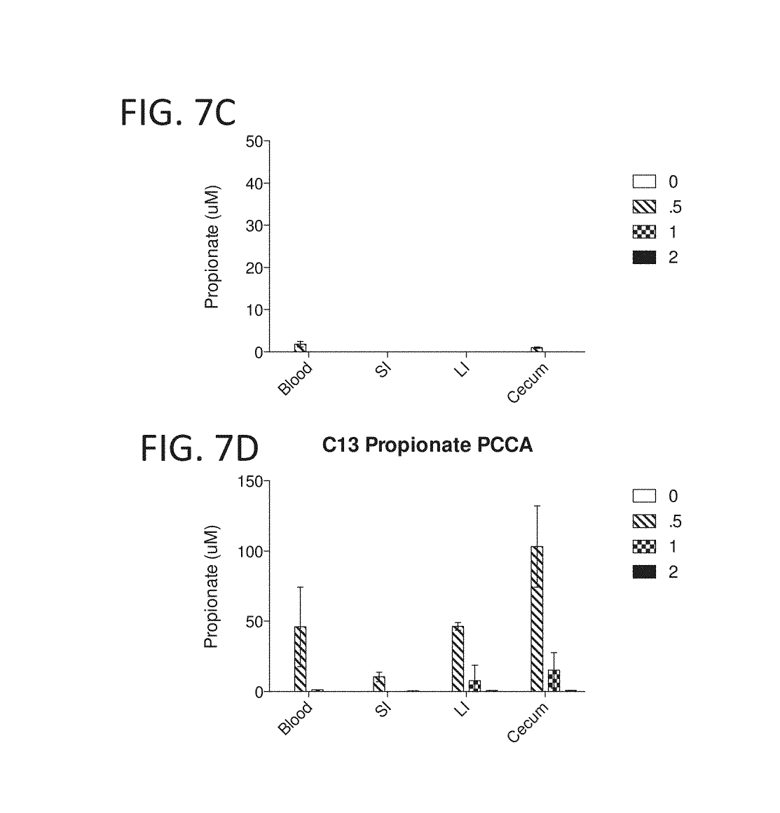

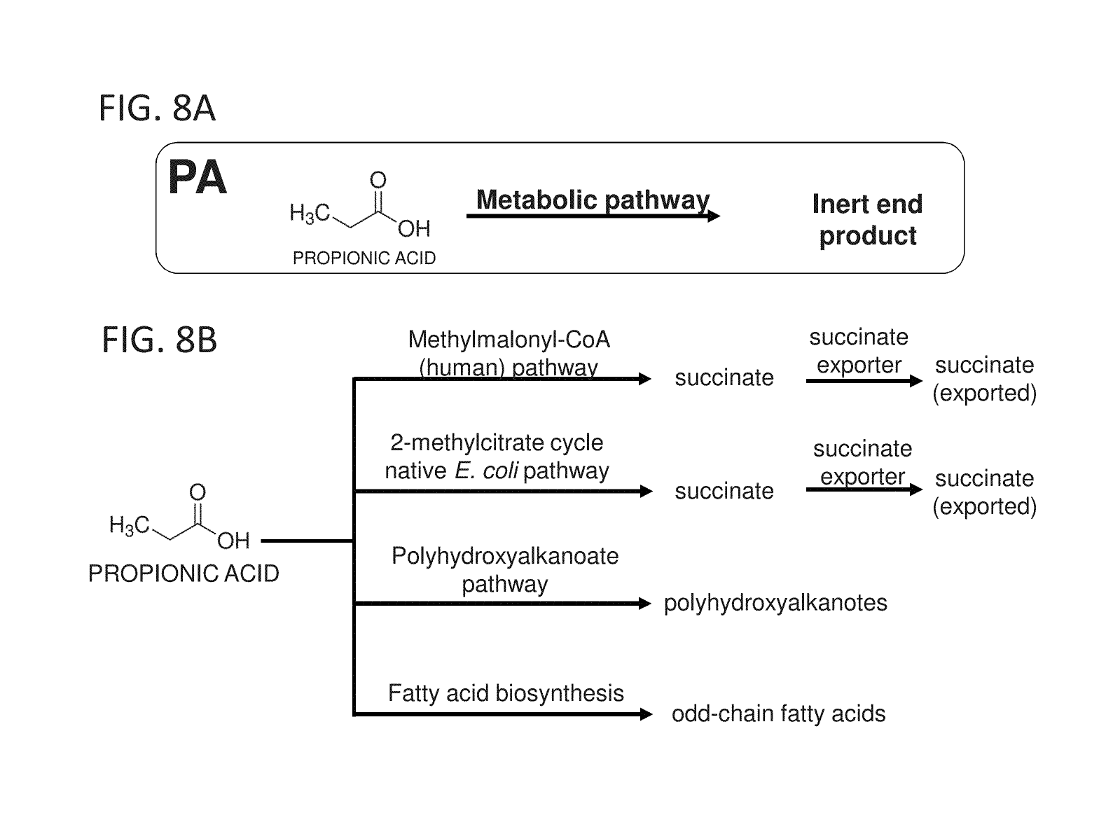

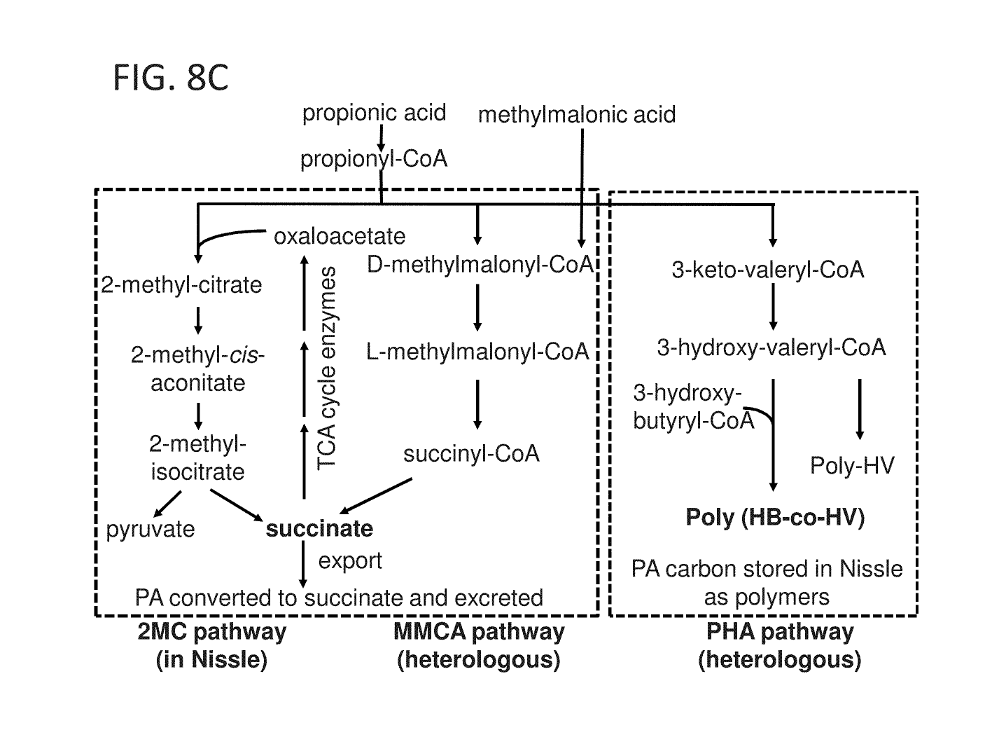

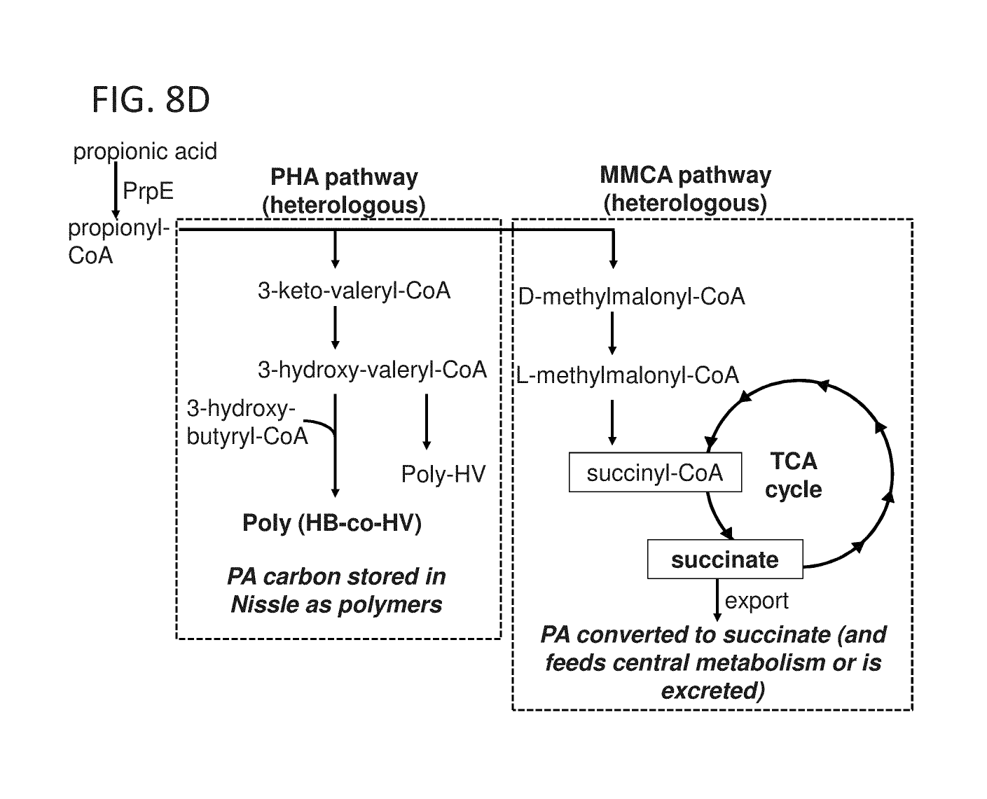

At each timepoint (0, 30 min, 1 h and 2 h post-SC injection), animals were euthanized, and blood, small intestine, large intestine and cecum, were removed and collected. Each intestinal section was flushed with 0.5 ml cold PBS and collected in separate 1.5 ml tubes. The cecum was harvested, contents were squeezed out, and flushed with 0.5 ml cold PBS and collected in a 1.5 ml tube. Blood was collected by mandibular bleeding. Concentrations of endogenous and radiolabeled propionate in the blood, intestinal compartments, and cecum were measured by LC-MS/MS as described herein. As shown in FIG. 7A-FIG. 7D, isotopic propionic acid injected SC is seen at very low levels in the blood, small intestine, and cecum within 30 min, indicating that propionate has circulated from blood into the intestinal compartments in the PA/MMA animal model.

### Example 8. Bacterial Contribution to PA Biomarkers

Experiments with antibiotic-treated PA patients suggest that bacterial metabolism in the gut contributes ˜30% of the propionate. The bacterial contribution to levels of PA biomarkers are evaluated by measuring the effects of an antibiotic treatment which significantly reduces the microbiota population (>99.9%) in the PCCA^{A138T} model.

PCCAA138T mice are kept on normal chow until Day 1 of the study. On day 1, plasma, urine, fecal samples are taken and, antibiotics supplemented in water of half of the mice (Ampicillin (1 g/L), Vancomycin (0.5 g/L), Neomycin (1 g/L), Metronidazole (1 g/L)) On D8, plasma, urine, fecal samples (n=4) are taken and metabolite levels quantified by LC-MS/MS as described herein. Bacterial levels are quantified by qPCR using primers which amplify DNA from Nissle and total bacteria. Metabolites (propionate, propionylcarnitine/acetylcarnitine ratio; propionylcarnitine, 2-methylcitrate, acetylcarnitine, are quantified by LC-MS/MS as described herein.

### Example 9. Polyhydroxyalkanoate (PHA) Pathway Propionate Consumption Assay

PHA pathway is a heterologous bacterial pathway used for carbon storage as polymers, and was assessed for its ability to consume propionate.

As described herein, the E. coli Nissle prpE gene and phaBCA genes from Acinetobacter sp RA3849 (codon optimized for expression in E. coli Nissle) were placed under the control of an aTc-inducible promoter in a single operon in a high copy plasmid, as shown in FIG. 10C and FIG. 11. Corresponding construct sequences are listed in Table 29 in Example 2. Next, the rate of propionate consumption of genetically engineered bacteria comprising the prpE-phaBCA circuit was assessed in vitro.

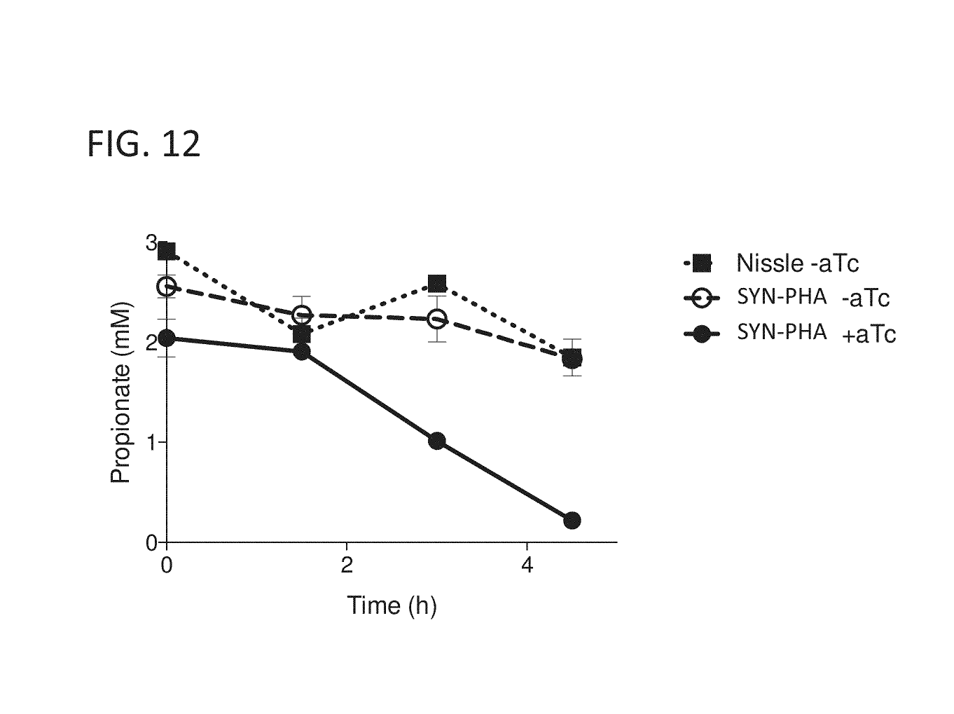

Cultures of E. coli Nissle transformed with the plasmid comprising the prpE-phaBCA circuit driven by the tet promoter and cultures of wild type control Nissle were grown overnight and then diluted 1:200 in LB. ATC was added to the cultures of the strain containing the prpE-phaBCA construct plasmid at a concentration of 100 ng/mL to induce expression of the prpE and phaBCA genes. Then, the cells were grown with shaking at 250 rpm. After 2 hrs of incubation, cells were pelleted down, washed, and resuspended in 1 mL M9 medium supplemented with glucose (0.2%) and propionate (2-8 mM) at a concentration of ˜10⁹ cfu/ml bacteria. Aliquots were collected at 0 hrs, 1.5 hr, 3 hrs, and 4.5 hrs for propionate quantification as described herein. As shown in FIG. 12, the genetically engineered bacteria expressing prpE and phaBCA genes driven by the tet promoter are more efficient at removing propionate than wild type Nissle or the uninduced engineered strain. The catabolic rate was calculated to be 0.396-1.4 umol hr⁻¹ per 10⁹ cells.

### Example 10. PHA Pathway Performance with Mixed Organic Acids

To determine whether acetate or butyrate (which are abundant in the colon) may have an effect on propionate consumption through the PHA pathway, the PHA assay was performed in a mixture of short chain fatty acids to mimic the colon ratios (propionate:acetate:butyrate, approximately 6:10:4).

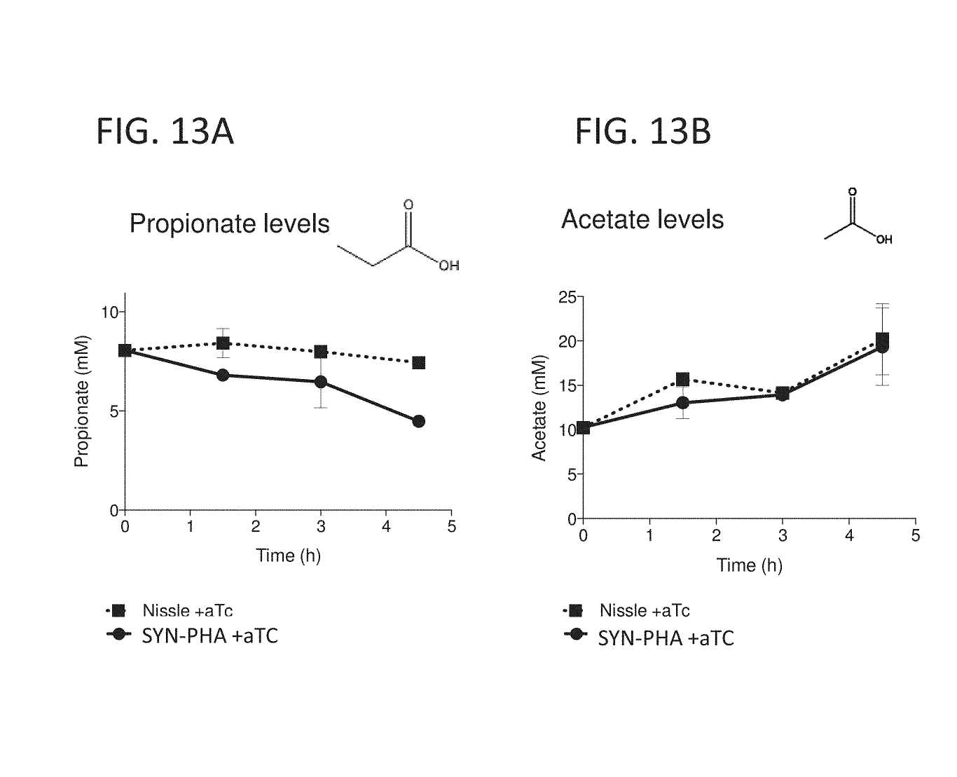

Cultures of E. coli Nissle transformed with the plasmid comprising the prpE-phaBCA circuit driven by the tet promoter (as described in Example 9) and wild type control Nissle were grown overnight and then diluted 1:200 in LB. ATC was added to the cultures of the strain containing the prpE-phaBCA construct plasmid and the wild type Nissle cultures and cells were incubated for two hours. Cells were spun down and resuspended in as described in Example 9 in 1 mL M9 medium supplemented with glucose (0.2%) and propionate (6 mM), butyrate (4 MM), and acetate (10 mM) at a concentration of ˜10⁹ cfu/ml bacteria. Aliquots were collected at 0 hrs, 1.5 hrs, 3 hrs, and 4.5 hrs for propionate quantification via LC-MS/MS as described herein. As shown in FIG. 13A, the genetically engineered bacteria expressing the tet-prpE and phaBCA gene cassette reduced the concentration of propionate compared to the wild type Nissle at a rate similar to the rate observed in the absence of acetate and butyrate in Example 9. The catabolic rate was calculated to be 0.396-1.4 umol hr-1 per 109 cells.

Also, the genetically engineered bacteria did not affect acetate or butyrate levels as compared to wild type Nissle (FIG. 13B and FIG. 13C), indicating that the PHA pathway does not significantly affect acetate and butyrate concentrations.

### Example 11. Optimization of the PHA Pathway

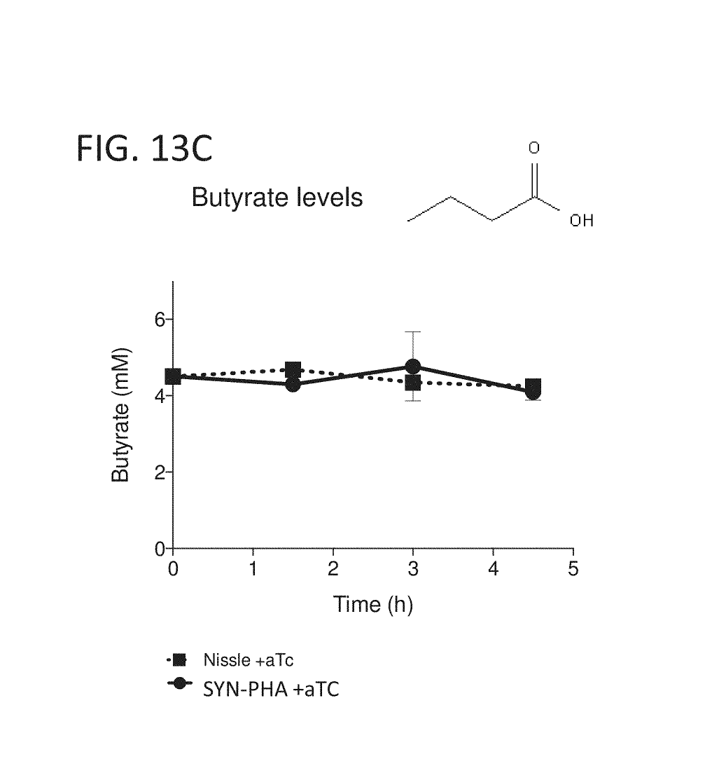

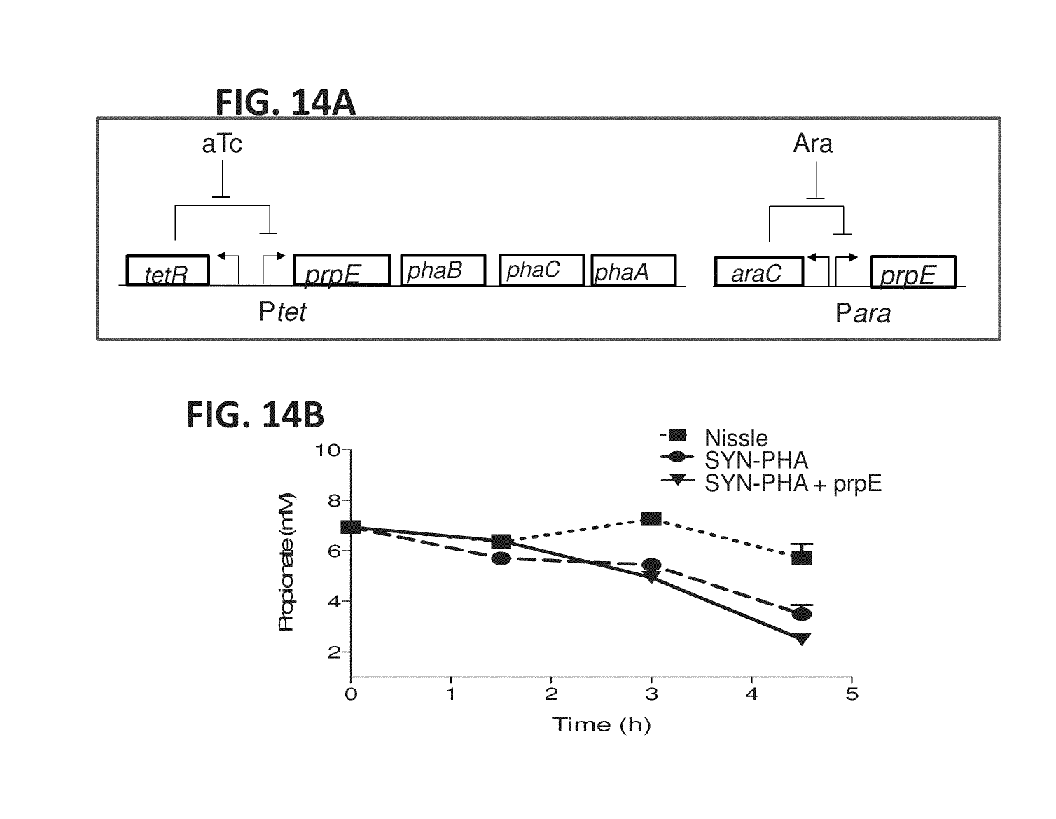

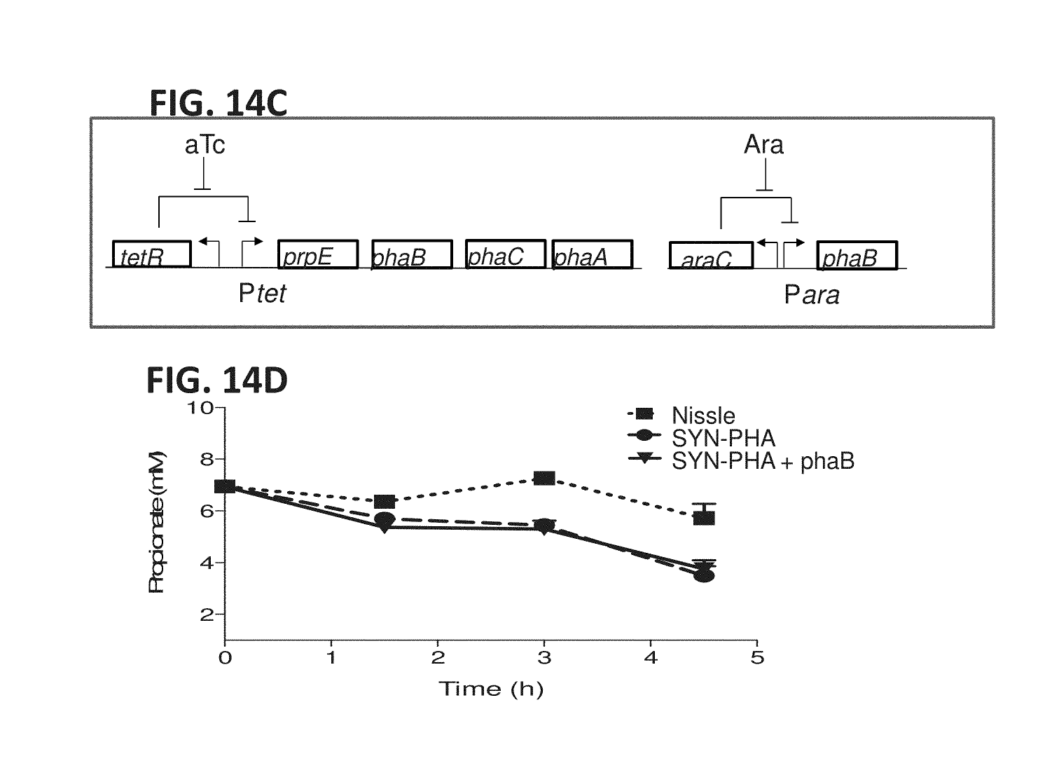

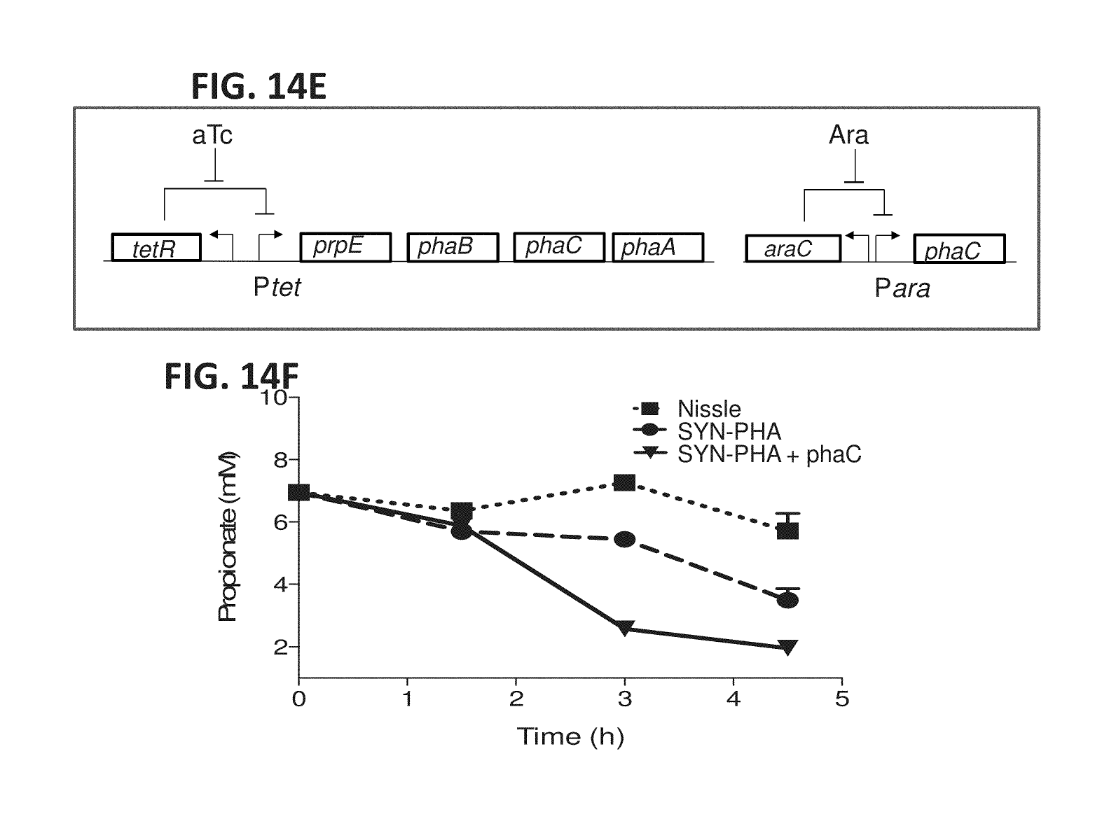

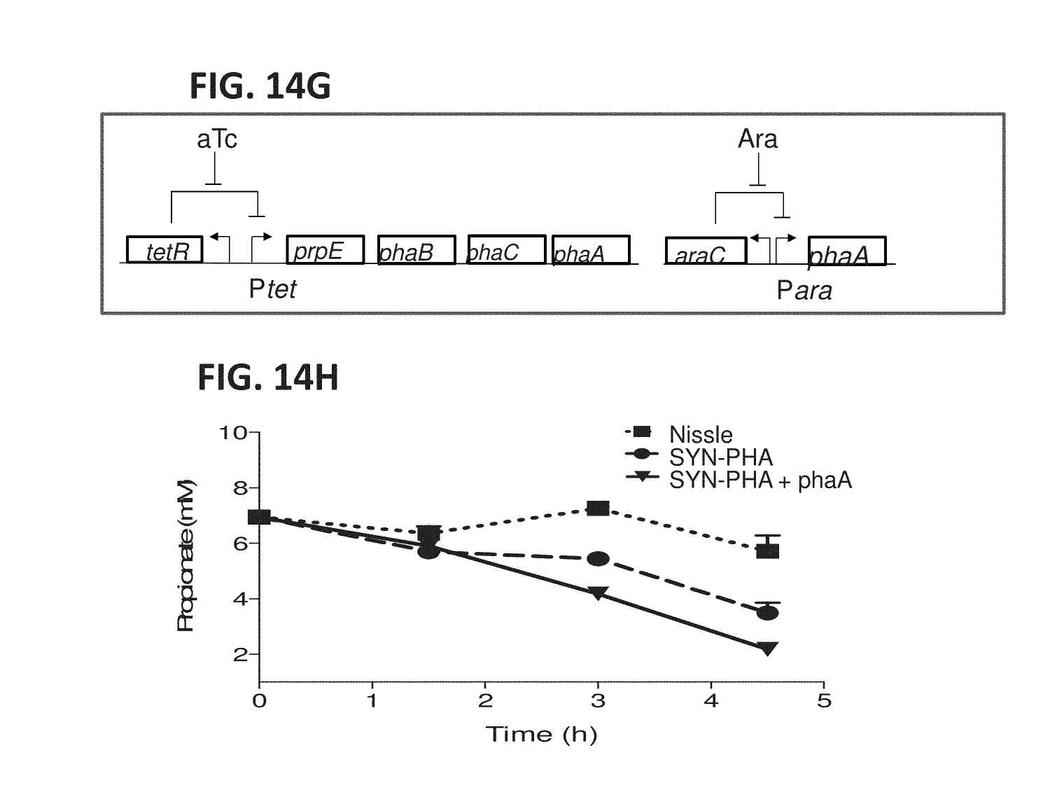

To optimize the PHA pathway and to determine the rate-limiting step in the pathway, the base strain expressing the aTc-inducible prpE-phaBCA operon was supplemented with a second plasmid expressing a construct containing one of the operon genes under the control of an arabinose inducible promoter, as shown in FIG. 14A-FIG. 14D. Table 31 lists the construct sequences from the additional plasmids.

In this assay, either the prpE-phaBCA operon alone, or both the prpE-phaBCA plasmid and the arabinose inducible plasmid carrying the additional copy of one of the genes in the pathway were induced to assess whether additional expression of any of the genes could increase propionate consumption. Wild type Nissle was included for reference.

TABLE 31
PHA Pathway Sequences - Additional Plasmid Constructs

| | | SEQ ID |
|---|---|---|
| Description | Sequence | NO |
| araC-Para-phaA | ttattcacaacctgccctaaactcgctcggactcgccccggtgcattttttaaatactc | SEQ ID |
| (araC: lower case; | gcgagaaatagagttgatcgtcaaaaccgacattgcgaccgacggtggcgatag | NO: 40 |
| RBS underlined; | gcatccgggtggtgctcaaaagcagcttcgcctgactgatgcgctggtcctcgcg | |
| phaA: italics; | ccagcttaatacgctaatccctaactgctggcggaacaaatgcgacagacgcgac | |
| L3S2P11 | ggcgacaggcagacatgctgtgcgacgctggcgatatcaaaattactgtctgcca | |
| terminator: | ggtgatcgctgatgtactgacaagcctcgcgtacccgattatccatcggtggatgg | |
| underlined bold; | agcgactcgttaatcgcttccatgcgccgcagtaacaattgctcaagcagatttatc | |
| his terminator: | gccagcaattccgaatagcgcccttccccttgtccggcattaatgatttgcccaaac | |
| bold) | aggtcgctgaaatgcggctggtgcgcttcatccgggcgaaagaaaccggtattgg | |
| | caaatatcgacggccagttaagccattcatgccagtaggcgcgcggacgaaagta | |
| | aacccactggtgataccattcgtgagcctccggatgacgaccgtagtgatgaatct | |
| | ctccaggcgggaacagcaaaatatcacccggtcggcagacaaattctcgtccctg | |
| | atttttcaccaccccctgaccgcgaatggtgagattgagaatataacctttcattccc | |
| | agcggtcggtcgataaaaaaatcgagataaccgttggcctcaatcggcgttaaacc | |
| | cgccaccagatgggcgttaaacgagtatcccggcagcaggggatcattttgcgctt | |
| | cagccatacttttcatactcccgccattcagagaagaaaccaattgtccatattgcat | |
| | CAGACATTGCCGTCACTGCGTCTTTTACTGGCTCT | |
| | TCTCGCTAACCCAACCGGTAACCCCGCTTATTAAA | |
| | AGCATTCTGTAACAAAGCGGGACCAAAGCCATGA | |
| | CAAAAACGCGTAACAAAAGTGTCTATAATCACGG | |
| | CAGAAAAGTCCACATTGATTATTTGCACGGCGTCA | |
| | CACTTTGCTATGCCATAGCATTTTTATCCATAAGA | |
| | TTAGCGGATCCAGCCTGACGCTTTTTTTCGCAACT | |
| | CTCTACTGTTTCTCCATACCATATTCATAGAAAGA | |
| | ATACTAAGAGAGGTCAGAATGAAAGATGTTGTTATC | |
| | GTAGCCGCTAAACGCACTGCGATCGGTTCCTTTCTGG | |
| | GGAGTCTGGCTTCCCTGAGCGCCCCTCAGTTGGGTC | |
| | AGACGGCTATCCGCGCAGTTTTGGATTCTGCAAATGT | |
| | GAAACCAGAACAAGTGGACCAAGTAATTATGGGGAAT | |
| | GTGCTGACCACCGGCGTTGGGCAAAATCCTGCTCGT | |
| | CAGGCAGCAATCGCCGCTGGGATTCCTGTACAAGTT | |
| | CCCGCCAGCACGCTTAATGTAGTGTGTGGGTCCGGA | |
| | TTACGTGCCGTTCACCTGGCAGCTCAAGCCATCCAAT | |
| | GCGATGAAGCCGATATCGTCGTTGCCGGAGGTCAAG | |
| | AATCAATGTCCCAGTCTGCTCATTACATGCAGCTTCG | |
| | CAATGGCCAGAAAATGGGTAACGCACAGTTAGTCGAT | |
| | TCAATGGTGGCCGACGGCTTGACCGACGCGTATAAT | |
| | CAATACCAGATGGGTATCACCGCGGAGAATATCGTCG | |
| | AAAAACTTGGTCTTAATCGTGAAGAACAAGACCAGCT | |
| | TGCTCTGACAAGTCAACAACGTGCTGCAGCAGCGCA | |
| | GGCTGCCGGAAAATTCAAGGATGAAATTGCGGTCGTT | |
| | TCGATTCCCCAGCGCAAAGGAGAGCCGGTCGTCTTC | |
| | GCGGAAGACGAATATATCAAGGCCAATACCTCGTTGG | |
| | AATCCTTGACGAAACTGCGTCCAGCATTCAAAAAAGA | |
| | CGGTTCTGTTACAGCCGGCAACGCATCTGGCATTAAT | |
| | GATGGGGCAGCCGCGGTCCTGATGATGTCCGCCGAC | |
| | AAAGCGGCTGAACTGGGCTTAAAGCCTTTAGCACGCA | |
| | TTAAAGGTTACGCGATGTCAGGAATTGAGCCGGAAAT | |
| | CATGGGACTGGGTCCTGTAGACGCCGTTAAGAAAAC | |
| | CCTTAATAAGGCTGGTTGGTCCTTAGACCAGGTCGAT | |
| | CTGATCGAGGCCAATGAGGCTTTTGCTGCCCAAGCA | |
| | CTGGGAGTAGCCAAGGAGCTTGGGCTGGACCTGGAC | |
| | AAGGTAAATGTTAACGGAGGTGCGATCGCGCTGGGA | |
| | CACCCGATCGGGGCTTCGGGTTGTCGTATCTTGGTC | |
| | ACGTTATTACACGAAATGCAGCGTCGTGATGCAAAGA | |
| | AGGGTATCGCCACATTGTGTGTGGGAGGTGGAATGG | |
| | GGGTGGCGCTTGCCGTTGAGCGCGATTAAGGAGCT | |
| | CGGTACCAAATTCCAGAAAAGAGACGCTTTCG | |
| | AGCGTCTTTTTTCGTTTTGGTCCGCGCAATAAA | |
| | AAAGCCCCCGGAAGGTGATCTTCCGGGGGCTT | |
| | TCTCATGCGTT | |
| araC-Para-phaB | Ttattcacaacctgccctaaactcgctcggactcgccccggtgcattttttaaatact | SEQ ID |
| (araC: lower case; | cgcgagaaatagagttgatcgtcaaaaccgacattgcgaccgacggtggcgata | NO: 41 |
| RBS underlined; | ggcatccgggtggtgctcaaaagcagcttcgcctgactgatgcgctggtcctcgc | |
| phaB: italics; | gccagcttaatacgctaatccctaactgctggcggaacaaatgcgacagacgcga | |
| L3S2P11 | cggcgacaggcagacatgctgtgcgacgctggcgatatcaaaattactgtctgcc | |
| terminator: | aggtgatcgctgatgtactgacaagcctcgcgtacccgattatccatcggtggatg | |
| underlined bold; | gagcgactcgttaatcgcttccatgcgccgcagtaacaattgctcaagcagatttat | |
| his terminator: | cgccagcaattccgaatagcgcccttccccttgtccggcattaatgatttgcccaaa | |
| bold) | caggtcgctgaaatgcggctggtgcgcttcatccgggcgaaagaaaccggtattg | |
| | gcaaatatcgacggccagttaagccattcatgccagtaggcgcgcggacgaaagt | |
| | aaacccactggtgataccattcgtgagcctccggatgacgaccgtagtgatgaatc | |
| | tctccaggcgggaacagcaaaatatcacccggtcggcagacaaattctcgtccct | |
| | gatttttcaccaccccctgaccgcgaatggtgagattgagaatataacctttcattcc | |
| | cagcggtcggtcgataaaaaaatcgagataaccgttggcctcaatcggcgttaaac | |
| | ccgccaccagatgggcgttaaacgagtatcccggcagcaggggatcattttgcgc | |
| | ttcagccatacttttcatactcccgccattcagagaagaaaccaattgtccatattgca | |
| | tCAGACATTGCCGTCACTGCGTCTTTTACTGGCTCT | |
| | TCTCGCTAACCCAACCGGTAACCCCGCTTATTAAA | |
| | AGCATTCTGTAACAAAGCGGGACCAAAGCCATGA | |
| | CAAAAACGCGTAACAAAAGTGTCTATAATCACGG | |
| | CAGAAAAGTCCACATTGATTATTTGCACGGCGTCA | |
| | CACTTTGCTATGCCATAGCATTTTTATCCATAAGA | |
| | TTAGCGGATCCAGCCTGACGCTTTTTTTCGCAACT | |
| | CTCTACTGTTTCTCCATAccGCTAGAACTAGATCTA | |
| | GAGTAATAAGGAGGAAGGAATGTCAGAGCAGAAAG | |
| | TAGCTCTGGTTACCGGTGCGTTAGGTGGTATCGGAA | |
| | GTGAGATCTGCCGCCAGCTTGTGACCGCCGGGTACA | |
| | AGATTATCGCCACCGTTGTTCCACGCGAAGAAGACCG | |
| | CGAAAAACAATGGTTGCAAAGTGAGGGGTTTCAAGAC | |
| | TCTGATGTGCGTTTCGTATTAACAGATTTAAACAATCA | |
| | CGAAGCTGCGACAGCGGCAATTCAAGAAGCGATTGC | |
| | CGCCGAAGGACGCGTTGATGTATTGGTCAACAACGC | |
| | GGGGATCACGCGCGATGCTACATTTAAGAAAATGTCC | |
| | TATGAGCAATGGTCCCAAGTCATCGACACGAATTTAA | |
| | AGACTCTTTTTACCGTGACCCAGCCAGTATTTAATAAA | |
| | ATGCTTGAACAGAAGTCTGGCCGCATCGTAAACATTA | |
| | GCTCTGTCAATGGTTTAAAAGGGCAATTTGGTCAAGC | |
| | CAACTACTCGGCCTCGAAAGCAGGGATTATCGGGTTT | |
| | ACTAAAGCATTGGCGCAGGAGGGTGCTCGCTCGAAC | |
| | ATTTGCGTCAATGTCGTTGCTCCTGGTTACACAGCGA | |
| | CACCCATGGTCACAGCAATGCGCGAGGATGTAATTAA | |
| | GTCAATCGAAGCTCAAATTCCCCTGCAACGTCTGGCA | |
| | GCACCGGCGGAGATTGCGGCAGCGGTTATGTATTTG | |
| | GTGAGTGAACACGGTGCATACGTGACGGGCGAAACT | |
| | TTGAGTATCAACGGCGGGCTGTACATGCACTAAGGA | |
| | GCTCGGTACCAAATTCCAGAAAAGAGACGCTTT | |
| | CGAGCGTCTTTTTTCGTTTTGGTCCGCGCAATA | |
| | AAAAAGCCCCCGGAAGGTGATCTTCCGGGGGC | |
| | TTTCTCATGCGTT | |
| acaC-Para-phaC | Ttattcacaacctgccctaaactcgctcggactcgccccggtgcattttttaaatact | SEQ ID |
| (araC: lower case; | cgcgagaaatagagttgatcgtcaaaaccgacattgcgaccgacggtggcgata | NO: 42 |
| RBS underlined; | ggcatccgggtggtgctcaaaagcagcttcgcctgactgatgcgctggtcctcgc | |
| phaC: italics; | gccagcttaatacgctaatccctaactgctggcggaacaaatgcgacagacgcga | |
| L3S2P11 | cggcgacaggcagacatgctgtgcgacgctggcgatatcaaaattactgtctgcc | |
| terminator: | aggtgatcgctgatgtactgacaagcctcgcgtacccgattatccatcggtggatg | |
| underlined bold; | gagcgactcgttaatcgcttccatgcgccgcagtaacaattgctcaagcagatttat | |
| his terminator: | cgccagcaattccgaatagcgcccttccccttgtccggcattaatgatttgcccaaa | |
| bold) | caggtcgctgaaatgcggctggtgcgcttcatccgggcgaaagaaaccggtattg | |
| | gcaaatatcgacggccagttaagccattcatgccagtaggcgcgcggacgaaagt | |
| | aaacccactggtgataccattcgtgagcctccggatgacgaccgtagtgatgaatc | |
| | tctccaggcgggaacagcaaaatatcacccggtcggcagacaaattctcgtccct | |
| | gatttttcaccaccccctgaccgcgaatggtgagattgagaatataacctttcattcc | |
| | cagcggtcggtcgataaaaaaatcgagataaccgttggcctcaatcggcgttaaac | |
| | ccgccaccagatgggcgttaaacgagtatcccggcagcaggggatcattttgcgc | |
| | ttcagccatacttttcatactcccgccattcagagaagaaaccaattgtccatattgca | |
| | tCAGACATTGCCGTCACTGCGTCTTTTACTGGCTCT | |
| | TCTCGCTAACCCAACCGGTAACCCCGCTTATTAAA | |
| | AGCATTCTGTAACAAAGCGGGACCAAAGCCATGA | |
| | CAAAAACGCGTAACAAAAGTGTCTATAATCACGG | |
| | CAGAAAAGTCCACATTGATTATTTGCACGGCGTCA | |
| | CACTTTGCTATGCCATAGCATTTTTATCCATAAGA | |
| | TTAGCGGATCCAGCCTGACGCTTTTTTTCGCAACT | |
| | CTCTACTGTTTCTCCATACCACTATTATTTAATATA | |
| | CGACATCAGGAGGTTCCAATGAATCCAAATTCCTTT | |
| | CAGTTTAAAGAGAATATCTTACAGTTTTTCAGCGTGCA | |
| | CGACGATATTTGGAAAAAACTGCAGGAATTTTACTATG | |
| | GACAATCGCCCATCAATGAAGCGTTGGCGCAGTTAAA | |
| | TAAGGAAGACATGAGTTTATTCTTCGAGGCGTTATCAA | |
| | AAAACCCTGCTCGTATGATGGAGATGCAGTGGTCCTG | |
| | GTGGCAAGGGCAGATTCAAATTTACCAGAACGTGTTA | |
| | ATGCGTAGTGTAGCCAAGGACGTAGCCCCCTTTATCC | |
| | AGCCAGAGTCCGGAGATCGTCGCTTCAACTCGCCAC | |
| | TTTGGCAAGAACATCCAAATTTTGATTTACTGAGTCAA | |
| | TCCTACTTGTTGTTTTCTCAGTTGGTTCAAAATATGGT | |
| | GGATGTCGTTGAAGGAGTACCTGATAAGGTCCGCTAT | |
| | CGCATCCATTTCTTTACACGTCAGATGATCAATGCGTT | |
| | GTCTCCTTCTAATTTCCTGTGGACGAACCCTGAAGTA | |
| | ATTCAACAGACGGTCGCTGAACAGGGTGAGAATTTAG | |
| | TACGCGGGATGCAAGTATTTCACGATGATGTAATGAA | |
| | TTCGGGTAAATATTTGAGCATCCGTATGGTAAATAGC | |
| | GACAGTTTCTCTCTTGGCAAGGACTTGGCGTATACGC | |
| | CAGGAGCCGTAGTTTTCGAGAACGACATCTTTCAGCT | |
| | TCTTCAATACGAAGCCACAACCGAGAACGTATATCAA | |
| | ACCCCTATTCTTGTCGTACCTCCCTTCATCAACAAGTA | |
| | CTACGTGCTGGACCTGCGCGAACAGAATAGCTTGGTT | |
| | AATTGGCTGCGCCAACAAGGACATACGGTGTTTTTGA | |
| | TGTCGTGGCGTAACCCCAACGCAGAGCAGAAGGAGC | |
| | TTACCTTCGCTGACTTAATTACCCAAGGATCGGTAGA | |
| | AGCATTACGTGTTATCGAAGAAATCACGGGAGAGAAA | |
| | GAAGCTAACTGTATTGGATATTGCATCGGTGGTACAC | |
| | TTCTGGCTGCTACCCAGGCATATTATGTAGCTAAACG | |
| | CCTGAAAAATCACGTAAAGTCAGCGACTTATATGGCG | |
| | ACGATTATTGATTTTGAGAACCCCGGCTCATTGGGTG | |
| | TTTTCATTAATGAGCCGGTCGTAAGTGGACTTGAAAA | |
| | CCTTAATAATCAACTTGGTTACTTCGACGGGCGTCAA | |
| | CTTGCAGTGACATTTTCGTTGTTGCGCGAAAACACCT | |
| | TGTATTGGAATTATTACATCGATAATTACTTGAAGGGT | |
| | AAGGAACCGTCCGACTTTGACATCTTATACTGGAACT | |
| | CGGATGGTACGAATATCCCAGCAAAGATTCACAATTT | |
| | CCTGTTACGTAACCTTTATCTTAACAACGAACTTATTT | |
| | CTCCAAATGCCGTCAAAGTTAATGGTGTGGGTTTAAA | |
| | CCTTTCGCGCGTGAAGACTCCATCATTCTTCATTGCTA | |
| | CGCAGGAGGACCATATCGCATTGTGGGATACCTGTTT | |
| | TCGCGGCGCGGATTACCTGGGGGGTGAGAGCACACT | |
| | TGTGCTTGGGGAAAGCGGACACGTCGCCGGCATTGT | |
| | CAACCCGCCTTCTCGTAACAAGTATGGTTGTTACACG | |
| | AACGCCGCCAAGTTTGAAAATACCAAGCAATGGCTTG | |
| | ACGGTGCAGAATATCATCCCGAAAGCTGGTGGTTACG | |
| | TTGGCAGGCATGGGTCACGCCTTATACTGGAGAGCA | |
| | GGTTCCTGCGCGTAATTTGGGAAACGCACAGTACCC | |
| | CAGTATTGAAGCGGCCCCTGGGCGTTATGTGCTGGT | |
| | AAACCTGTTTTAAGGAGCTCGGTACCAAATTCCAG | |
| | AAAAGAGACGCTTTCGAGCGTCTTTTTTCGTTT | |
| | TGGTCCGCGCAATAAAAAAGCCCCCGGAAGGT | |
| | GATCTTCCGGGGGCTTTCTCATGCGTT | |
| AraC-pAra-PrpE | ttattcacaacctgccctaaactcgctcggactcgccccggtgcattttttaaatactc | SEQ ID |
| (AraC: Lower | gcgagaaatagagttgatcgtcaaaaccgacattgcgaccgacggtggcgatag | NO: 43 |
| Case; RBS | gcatccgggtggtgctcaaaagcagcttcgcctgactgatgcgctggtcctcgcg | |
| Underlined; PrpE: | ccagcttaatacgctaatccctaactgctggcggaacaaatgcgacagacgcgac | |
| Italics; L3s2p11 | ggcgacaggcagacatgctgtgcgacgctggcgatatcaaaattactgtctgcca | |
| Terminator: | ggtgatcgctgatgtactgacaagcctcgcgtacccgattatccatcggtggatgg | |
| Underlined; His | agcgactcgttaatcgcttccatgcgccgcagtaacaattgctcaagcagatttatc | |
| Terminator: Bold) | gccagcaattccgaatagcgcccttccccttgtccggcattaatgatttgcccaaac | |
| | aggtcgctgaaatgcggctggtgcgcttcatccgggcgaaagaaaccggtattgg | |
| | caaatatcgacggccagttaagccattcatgccagtaggcgcgcggacgaaagta | |
| | aacccactggtgataccattcgtgagcctccggatgacgaccgtagtgatgaatct | |
| | ctccaggcgggaacagcaaaatatcacccggtcggcagacaaattctcgtccctg | |
| | atttttcaccaccccctgaccgcgaatggtgagattgagaatataacctttcattccc | |
| | agcggtcggtcgataaaaaaatcgagataaccgttggcctcaatcggcgttaaacc | |
| | cgccaccagatgggcgttaaacgagtatcccggcagcaggggatcattttgcgctt | |
| | cagccatacttttcatactcccgccattcagagaagaaaccaattgtccatattgcat | |
| | CAGACATTGCCGTCACTGCGTCTTTTACTGGCTCT | |
| | TCTCGCTAACCCAACCGGTAACCCCGCTTATTAAA | |
| | AGCATTCTGTAACAAAGCGGGACCAAAGCCATGA | |
| | CAAAAACGCGTAACAAAAGTGTCTATAATCACGG | |
| | CAGAAAAGTCCACATTGATTATTTGCACGGCGTCA | |
| | CACTTTGCTATGCCATAGCATTTTTATCCATAAGA | |
| | TTAGCGGATCCAGCCTGACGCTTTTTTTCGCAACT | |
| | CTCTACTGTTTCTCCATACCAGATTTAAAGTAAGG | |
| | CCAGGGAATAAATGTCTTTTAGCGAATTTTATCAGCG | |
| | TTCGATTAACGAACCGGAGAAGTTCTGGGCCGAGCA | |
| | GGCCCGGCGTATTGACTGGCAGACGCCCTTTACGCA | |
| | AACGCTCGACCACAGCAACCCGCCGTTTGCCCGTTG | |
| | GTTTTGTGAAGGCCGAACCAACTTGTGTCACAACGCT | |
| | ATCGACCGCTGGCTGGAGAAACAGCCAGAGGCGCTG | |
| | GCATTGATTGCCGTCTCTTCGGAAACAGAGGAAGAGC | |
| | GTACCTTTACCTTCCGCCAGTTACATGACGAAGTGAA | |
| | TGCGGTGGCGTCAATGCTGCGCTCACTGGGCGTGCA | |
| | GCGTGGCGATCGGGTGCTGGTGTATATGCCGATGAT | |
| | TGCCGAAGCGCATATTACCCTGCTGGCCTGCGCGCG | |
| | CATTGGTGCTATTCACTCGGTGGTGTTTGGGGGATTT | |
| | GCTTCGCACAGCGTGGCAACGCGAATTGATGACGCT | |
| | AAACCGGTGCTGATTGTCTCGGCTGATGCCGGGGCG | |
| | CGCGGCGGTAAAATCATTCCGTATAAAAAATTGCTCG | |
| | ACGATGCGATAAGTCAGGCACAGCATCAGCCGCGTC | |
| | ACGTTTTACTGGTGGATCGCGGGCTGGCGAAAATGG | |
| | CGCGCGTTAGCGGGCGGGATGTCGATTTCGCGTCGT | |
| | TGCGCCATCAACACATCGGCGCGCGGGTGCCGGTG | |
| | GCATGGCTGGAATCCAACGAAACCTCCTGCATTCTCT | |
| | ACACCTCCGGCACGACCGGCAAACCTAAAGGTGTGC | |
| | AGCGTGATGTCGGCGGATATGCGGTGGCGCTGGCG | |
| | ACCTCGATGGACACCATTTTTGGCGGCAAAGCGGGC | |
| | GGCGTGTTCTTTTGTGCTTCGGATATCGGCTGGGTGG | |
| | TAGGGCATTCGTATATCGTTTACGCGCCGCTGCTGGC | |
| | GGGGATGGCGACTATCGTTTACGAAGGATTGCCGAC | |
| | CTGGCCGGACTGCGGCGTGTGGTGGAAAATTGTCGA | |
| | GAAATATCAGGTTAGCCGCATGTTCTCAGCGCCGACC | |
| | GCCATTCGCGTGCTGAAAAAATTCCCTACCGCTGAAA | |
| | TTCGCAAACACGATCTTTCGTCGCTGGAAGTGCTCTA | |
| | TCTGGCTGGAGAACCGCTGGACGAGCCGACCGCCA | |
| | GTTGGGTGAGCAATACGCTGGATGTGCCGGTCATCG | |
| | ACAACTACTGGCAGACCGAATCCGGCTGGCCGATTAT | |
| | GGCGATTGCTCGCGGTCTGGATGACAGACCGACGCG | |
| | TCTGGGAAGCCCCGGCGTGCCGATGTATGGCTATAA | |
| | CGTGCAGTTGCTCAATGAAGTCACCGGCGAACCGTG | |
| | TGGCGTCAATGAGAAAGGGATGCTGGTAGTGGAGGG | |
| | GCCATTGCCGCCAGGCTGTATTCAAACCATCTGGGG | |
| | CGACGACGACCGCTTTGTGAAGACGTACTGGTCGCT | |
| | GTTTTCCCGTCCGGTGTACGCCACTTTTGACTGGGGC | |
| | ATCCGCGATGCTGACGGTTATCACTTTATTCTCGGGC | |
| | GCACTGACGATGTGATTAACGTTGCCGGACATCGGCT | |
| | GGGTACGCGTGAGATTGAAGAGAGTATCTCCAGTCAT | |
| | CCGGGCGTTGCCGAAGTGGCGGTGGTTGGGGTGAA | |
| | AGATGCGCTGAAAGGGCAGGTGGCGGTGGCGTTTGT | |
| | CATTCCGAAAGAGAGCGACAGTCTGGAAGACCGTGA | |
| | GGTGGCGCACTCGCAAGAGAAGGCGATTATGGCGCT | |
| | GGTGGACAGCCAGATTGGCAACTTTGGCCGCCCGGC | |
| | GCACGTCTGGTTTGTCTCGCAATTGCCAAAAACGCGA | |
| | TCCGGAAAAATGCTGCGCCGCACGATCCAGGCGATT | |
| | TGCGAAGGACGCGATCCTGGGGATCTGACGACCATT | |
| | GATGATCCGGCGTCGTTGGATCAGATCCGCCAGGCG | |
| | ATGGAAGAGTAGGGAGCTCGGTACCAAATTCCAG | |
| | AAAAGAGACGCTTTCGAGCGTCTTTTTTCGTTT | |
| | TGGTCCGCGCAATAAAAAAGCCCCCGGAAGGT | |
| | GATCTTCCGGGGGCTTTCTCATGCGTT | |

Cultures of E. coli Nissle transformed with the plasmid comprising the tet-prpE-phaBCA circuit and the second plasmid (containing one of pAra-prpE or pAra-phaB or pAra-phaC or pAra-phaA) were grown overnight and then diluted 1:200 in LB. Wild type control Nissle cultures were also grown as a reference. ATC (100 ng/mL) was added to induce the tet-prpE-phaBCA construct gene cassette. In half of the cultures of the four strains containing the tet-prpE-phaBCA circuit, arabinose was added at a concentration of 10 mM to induce the second plasmid. Cells were grown with shaking at 250 rpm. After 2 hrs of incubation, cells were pelleted down, washed, and resuspended in 1 mL M9 medium 0.5% glucose 8 mM propionate added at a concentration of ˜10⁹ cfu/ml bacteria. Aliquots were collected at 0 hrs, 1 hrs, 2 hrs, 3 hrs, 4 hrs, and 5 hrs for propionate quantification by LC-MS/MS. As shown in FIG. 14A-FIG. 14D, the rate of propionate consumption is increased most significantly when more phaC is expressed, suggesting that the pathway is improved by increasing the PhaC levels from the original prpE-phaBCA plasmid.

In certain embodiments, the prpE-phaBCA circuit is further modified by adding a strong RBS upstream of the phaC translation start site. In certain embodiments, the genetically engineered bacteria comprise one or more prpE-phaBCA gene cassettes and one or more additional cassettes comprising the phaA gene.

### Example 12. In Vitro Activity of the MMCA Pathway

The methylmalonyl-CoA pathway was assessed in vitro for its ability to catabolize propionate. As described in Example 3, genes accA (from Streptomyces coelicolor), pccB (from Streptomyces coelicolor), mmcE (from Propionibacterium freudenreichii), and mutAB (from Propionibacterium freudenreichii) were codon-optimized for expression in E. coli Nissle. Two plasmids, the first plasmid with a cassette comprising prpE, pccB, accA1, under the control of an inducible Ptet promoter and the second plasmid with a cassette comprising mmcE and mutAB under the control of a second inducible promoter, Para, were generated (as shown in FIG. 15C and FIG. 16A and FIG. 16B). Induction of the pathway therefore requires the addition of aTc and arabinose. Sequences of MMCA pathway circuits are listed in Table 30 in Example 3.

Cultures of E. coli Nissle comprising the first and second plasmids with the MMCA circuits and wild type control Nissle, were grown overnight in LB and 50 ug/mL Ampicillin and then diluted 1:100 in LB. The cells were grown with shaking (250 rpm) to early log phase with the appropriate antibiotics. Anhydrous tetracycline (ATC) and arabinose (10 mM) was added to cultures at a concentration of 100 ng/mL to induce expression of the constructs, and bacteria were grown for another 2 hours. Bacteria were then pelleted, washed, and resuspended in minimal media at ˜10⁹ cfu/ml, and supplemented with 0.5% glucose and propionate (6 mM). Aliquots were removed at 0 hrs, 2 hrs, 4, hrs, 17, hrs and 18 hrs for propionate quantification by LC-MS/MS analysis.

For induction of the PHA pathway, cultures were grown, induced, and assayed as described in Example 9.

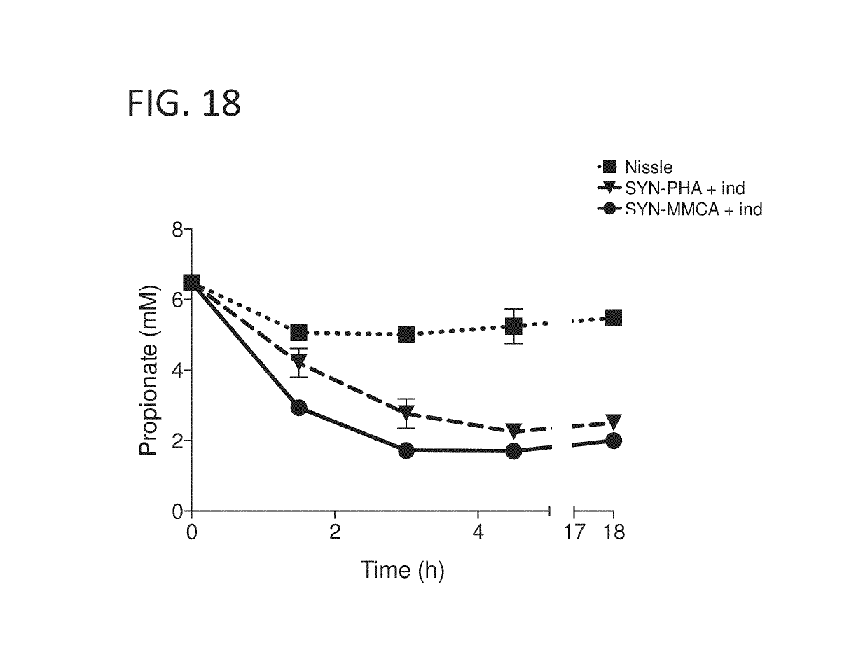

As shown in FIG. 18, the expression of the MMCA circuits reduces the propionate concentration in the media, indicating that the circuits promote propionate catalysis. Propionate assay was initiated with ˜10⁹ cfu/ml pre-induced bacteria and the propionate consumption rate was ˜3.8 μM/hr/10⁹ bacteria in the strain expressing the methylmalonyl-CoA pathway circuit. Overall the MMCA pathway seems more effective at propionate breakdown than the PHA pathway.

### Example 13. In Vitro Activity of the MMCA Pathway Circuit in Combination with a Succinate Exporter Circuit

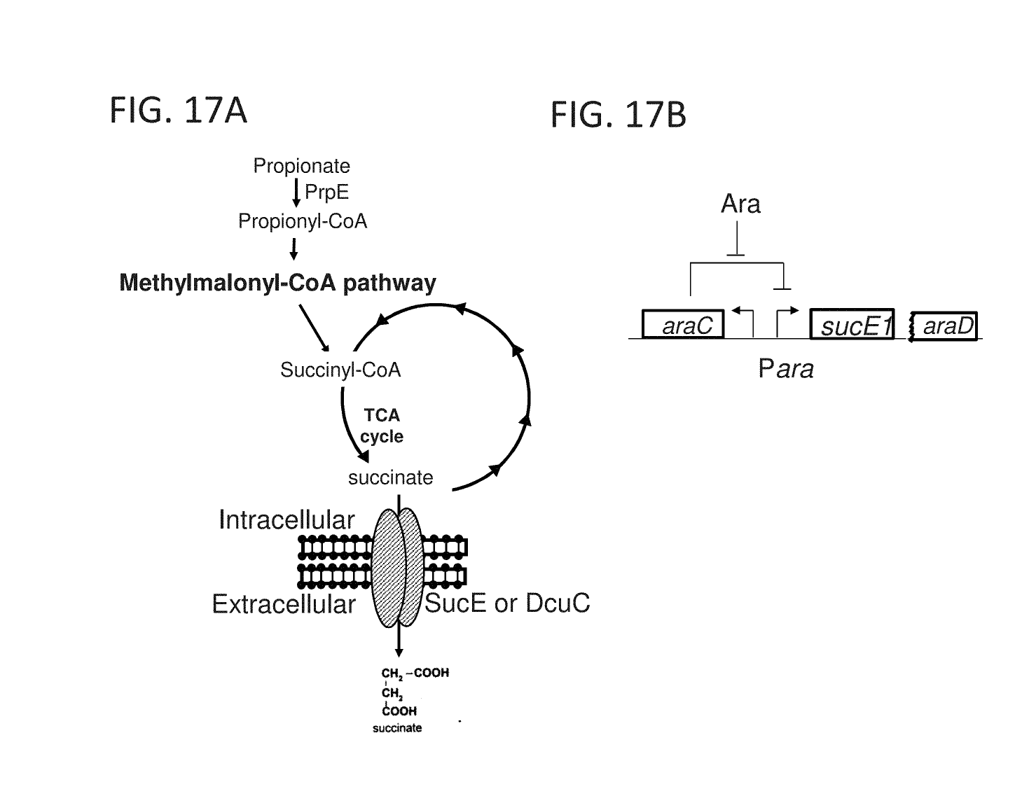

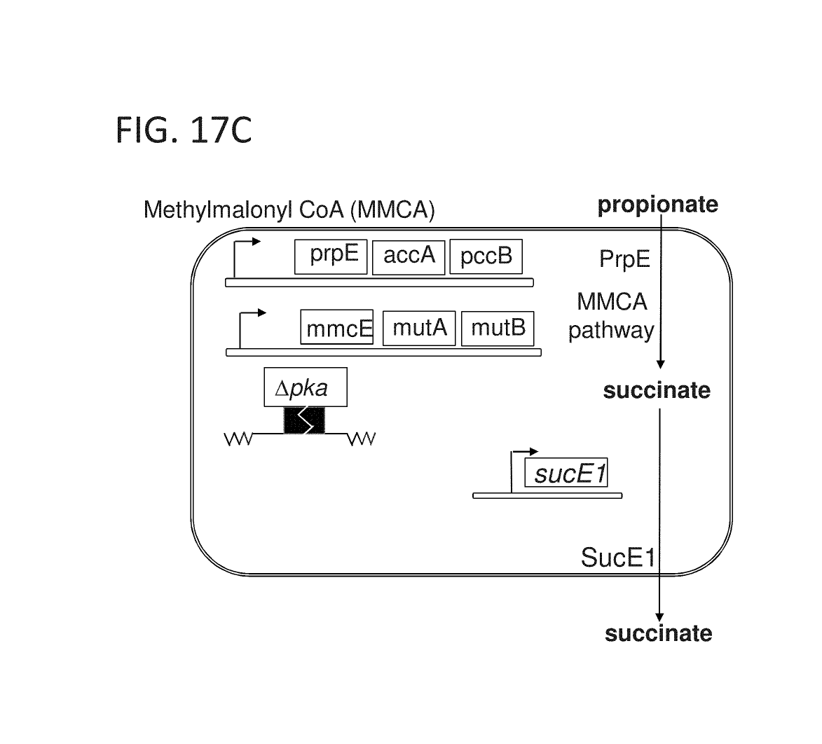

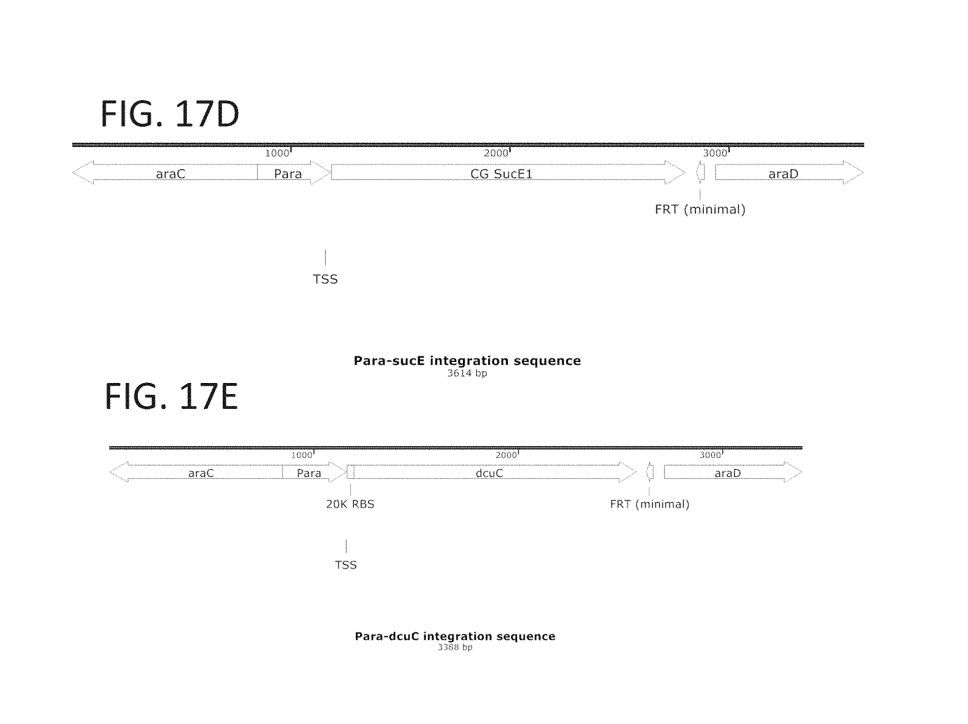

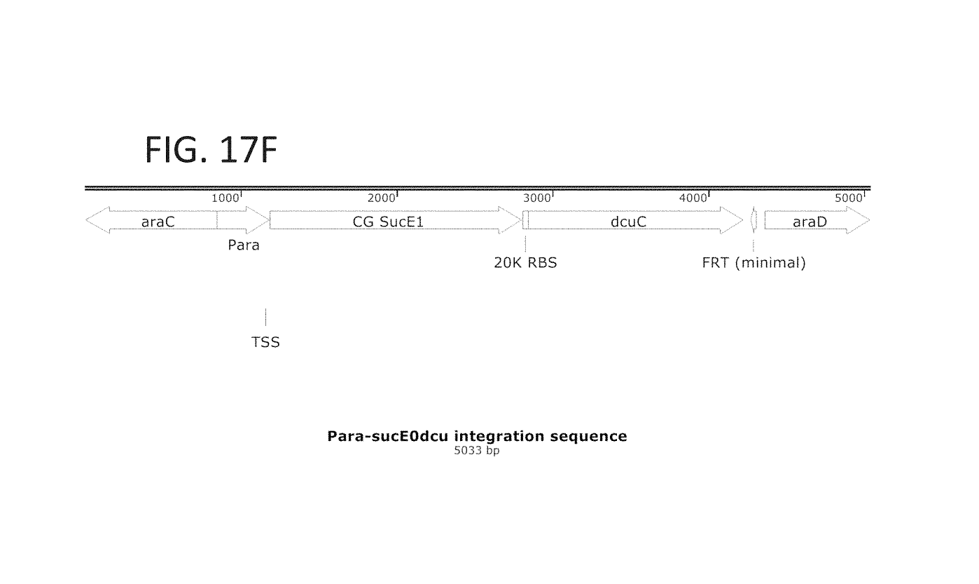

In order to determine whether a succinate exporter may increase the amount of propionate catabolized through the MMCA pathway, a construct was generated comprising the sucE1 succinate exporter (from Corynebacterium glutamicum (as shown in FIG. 17B and FIG. 17D) or the E. coli dcuC succinate transporter (FIG. 17E) or comprising both transporters (FIG. 17F). The sucE1 construct was placed under the control of Para (arabinose-inducible) in the Nissle chromosome. This knock-in also deleted the araBA genes as well as part of the araD gene, effectively eliminating metabolism of arabinose by E. coli.

Sequences of the exporter constructs are shown in Table 32. In vitro activity of MMCA pathway circuit is compared alone or in combination with an integrated sucE1 circuit, essentially as described in Example 12 and elsewhere herein.

TABLE 32
Succinate Exporter Construct Sequences

| | | SEQ ID |
|---|---|---|
| Description | Sequence | NO |
| pAraC-SucE1 | Ttattcacaacctgccctaaactcgctcggactcgccccggtgcattttttaaatactcgc | SEQ ID |
| (as shown in | gagaaatagagttgatcgtcaaaaccgacattgcgaccgacggtggcgataggcatcc | NO: 44 |
| FIG. 17D; | gggtggtgctcaaaagcagcttcgcctgactgatgcgctggtcctcgcgccagcttaata | |
| AraC: lower | cgctaatccctaactgctggcggaacaaatgcgacagacgcgacggcgacaggcaga | |
| case; pARA: | catgctgtgcgacgctggcgatatcaaaattactgtctgccaggtgatcgctgatgtactg | |
| upper case | acaagcctcgcgtacccgattatccatcggtggatggagcgactcgttaatcgcttccat | |
| italics; RBS: | gcgccgcagtaacaattgctcaagcagatttatcgccagcaattccgaatagcgcccttc | |
| underlined; | cccttgtccggcattaatgatttgcccaaacaggtcgctgaaatgcggctggtgcgcttca | |
| sucE1: bold; | tccgggcgaaagaaaccggtattggcaaatatcgacggccagttaagccattcatgcca | |
| FRT minimal: | gtaggcgcgcggacgaaagtaaacccactggtgataccattcgtgagcctccggatga | |
| underline italics) | cgaccgtagtgatgaatctctccaggcgggaacagcaaaatatcacccggtcggcaga | |
| | caaattctcgtccctgatttttcaccaccccctgaccgcgaatggtgagattgagaatataa | |
| | cctttcattcccagcggtcggtcgataaaaaaatcgagataaccgttggcctcaatcggc | |
| | gttaaacccgccaccagatgggcgttaaacgagtatcccggcagcaggggatcattttg | |
| | cgcttcagccatACTTTTCATACTCCCGCCATTCAGAGAAGAAA | |
| | CCAATTGTCCATATTGCATCAGACATTGCCGTCACTGCGT | |
| | CTTTTACTGGCTCTTCTCGCTAACCCAACCGGTAACCCC | |
| | GCTTATTAAAAGCATTCTGTAACAAAGCGGGACCAAAGC | |
| | CATGACAAAAACGCGTAACAAAAGTGTCTATAATCACGGC | |
| | AGAAAAGTCCACATTGATTATTTGCACGGCGTCACACTTT | |
| | GCTATGCCATAGCATTTTTATCCATAAGATTAGCGGATCC | |
| | AGCCTGACGCTTTTTTTCGCAACTCTCTACTGTTTCTCCA | |
| | TACCCGTTTTTTTGGATGGAGTGAAACGATGTCCTTC | |
| | CTGGTCGAGAATCAATTGTTAGCACTTGTCGTGAT | |
| | CATGACCGTCGGGCTTTTACTTGGACGTATCAAAA | |
| | TCTTTGGTTTCCGTTTGGGTGTGGCCGCCGTGTTG | |
| | TTCGTCGGCCTTGCTTTAAGCACCATTGAGCCCGA | |
| | CATTTCGGTTCCATCCCTTATTTACGTGGTTGGCC | |
| | TTTCGCTTTTTGTGTATACTATCGGTCTGGAAGCT | |
| | GGCCCCGGTTTTTTTACATCTATGAAGACGACGGG | |
| | TTTGCGCAATAACGCACTGACGTTAGGTGCCATTA | |
| | TCGCGACAACAGCACTTGCGTGGGCACTGATTAC | |
| | CGTCTTGAATATTGATGCCGCCTCAGGAGCTGGTA | |
| | TGCTTACTGGTGCCTTAACTAATACGCCCGCTATG | |
| | GCTGCGGTAGTGGATGCACTTCCCTCATTAATTGA | |
| | TGACACAGGCCAGCTGCATCTTATTGCTGAGCTGC | |
| | CGGTGGTTGCTTATTCCCTGGCTTATCCCTTGGGG | |
| | GTACTGATTGTGATCTTGAGCATCGCCATCTTTTC | |
| | TTCAGTGTTTAAGGTTGACCATAACAAGGAGGCAG | |
| | AAGAGGCTGGGGTAGCGGTCCAAGAACTTAAGGG | |
| | CCGCCGTATCCGCGTAACTGTAGCTGACTTGCCAG | |
| | CCCTTGAGAACATTCCTGAGTTGCTTAATTTACAT | |
| | GTTATCGTCTCGCGTGTAGAGCGCGACGGAGAGC | |
| | AGTTCATCCCCTTATATGGCGAACATGCACGCATC | |
| | GGCGATGTACTGACTGTCGTGGGGGCCGACGAGG | |
| | AACTGAACCGCGCGGAAAAAGCCATCGGAGAGTT | |
| | AATTGACGGTGATCCTTACTCTAACGTTGAACTGG | |
| | ACTATCGTCGTATCTTCGTCTCTAATACGGCGGTT | |
| | GTCGGTACACCCCTGAGCAAATTGCAACCGCTTTT | |
| | TAAAGATATGCTTATTACTCGCATTCGCCGCGGTG | |
| | ATACGGATCTGGTAGCTTCCTCGGACATGACGCTT | |
| | CAATTAGGCGACCGCGTTCGTGTGGTTGCCCCAG | |
| | CCGAGAAACTTCGTGAAGCGACTCAGTTGCTTGG | |
| | AGACTCTTACAAAAAGCTGTCCGACTTTAATTTAT | |
| | TGCCTCTTGCTGCGGGCTTAATGATTGGCGTCCTT | |
| | GTTGGAATGGTTGAATTCCCACTGCCTGGGGGGT | |
| | CATCTTTAAAACTTGGCAATGCCGGTGGTCCGTTG | |
| | GTTGTCGCGCTGTTGCTTGGGATGATCAATCGTAC | |
| | GGGAAAGTTCGTCTGGCAGATCCCGTACGGAGCA | |
| | AACTTGGCGTTACGTCAGTTGGGTATCACCCTGTT | |
| | CTTGGCGGCTATTGGCACTTCCGCGGGAGCTGGG | |
| | TTTCGCTCAGCTATTAGCGACCCGCAATCTCTGAC | |
| | CATTATTGGATTTGGTGCGTTGTTAACCTTGTTTA | |
| | TTAGTATTACCGTCTTGTTCGTTGGGCATAAGTTG | |
| | ATGAAAATCCCGTTTGGGGAAACGGCGGGTATCT | |
| | TAGCTGGAACGCAGACCCATCCAGCAGTATTATCA | |
| | TATGTGTCTGACGCATCTCGCAACGAGTTGCCAGC | |
| | CATGGGGTACACCTCAGTGTATCCCTTGGCTATGA | |
| | TTGCGAAAATCCTGGCTGCACAAACACTTTTGTTT | |
| | CTGTTGATTtaatgaGGAATCGACTCCACGTCCCTAGCG | |
| | TGTGTAGGCTGGAGCTGCTTCGAAGTTCCTATACTTTCT | |
| | AGAGAATAGGAACTTC | |
| SucE1 with RBS | CCCGTTTTTTTGGATGGAGTGAAACGATGTCCTTCCT | SEQ ID |
| (underlined) | GGTCGAGAATCAATTGTTAGCACTTGTCGTGATCATG | NO: 45 |
| | ACCGTCGGGCTTTTACTTGGACGTATCAAAATCTTTG | |
| | GTTTCCGTTTGGGTGTGGCCGCCGTGTTGTTCGTCGG | |
| | CCTTGCTTTAAGCACCATTGAGCCCGACATTTCGGTT | |
| | CCATCCCTTATTTACGTGGTTGGCCTTTCGCTTTTTGT | |
| | GTATACTATCGGTCTGGAAGCTGGCCCCGGTTTTTTT | |
| | ACATCTATGAAGACGACGGGTTTGCGCAATAACGCA | |
| | CTGACGTTAGGTGCCATTATCGCGACAACAGCACTTG | |
| | CGTGGGCACTGATTACCGTCTTGAATATTGATGCCGC | |
| | CTCAGGAGCTGGTATGCTTACTGGTGCCTTAACTAAT | |
| | ACGCCCGCTATGGCTGCGGTAGTGGATGCACTTCCCT | |
| | CATTAATTGATGACACAGGCCAGCTGCATCTTATTGC | |
| | TGAGCTGCCGGTGGTTGCTTATTCCCTGGCTTATCCCT | |
| | TGGGGGTACTGATTGTGATCTTGAGCATCGCCATCTT | |
| | TTCTTCAGTGTTTAAGGTTGACCATAACAAGGAGGCA | |
| | GAAGAGGCTGGGGTAGCGGTCCAAGAACTTAAGGGC | |
| | CGCCGTATCCGCGTAACTGTAGCTGACTTGCCAGCCC | |
| | TTGAGAACATTCCTGAGTTGCTTAATTTACATGTTATC | |
| | GTCTCGCGTGTAGAGCGCGACGGAGAGCAGTTCATC | |
| | CCCTTATATGGCGAACATGCACGCATCGGCGATGTAC | |
| | TGACTGTCGTGGGGGCCGACGAGGAACTGAACCGCG | |
| | CGGAAAAAGCCATCGGAGAGTTAATTGACGGTGATC | |
| | CTTACTCTAACGTTGAACTGGACTATCGTCGTATCTTC | |
| | GTCTCTAATACGGCGGTTGTCGGTACACCCCTGAGCA | |
| | AATTGCAACCGCTTTTTAAAGATATGCTTATTACTCG | |
| | CATTCGCCGCGGTGATACGGATCTGGTAGCTTCCTCG | |
| | GACATGACGCTTCAATTAGGCGACCGCGTTCGTGTGG | |
| | TTGCCCCAGCCGAGAAACTTCGTGAAGCGACTCAGTT | |
| | GCTTGGAGACTCTTACAAAAAGCTGTCCGACTTTAAT | |
| | TTATTGCCTCTTGCTGCGGGCTTAATGATTGGCGTCCT | |
| | TGTTGGAATGGTTGAATTCCCACTGCCTGGGGGGTCA | |
| | TCTTTAAAACTTGGCAATGCCGGTGGTCCGTTGGTTG | |
| | TCGCGCTGTTGCTTGGGATGATCAATCGTACGGGAAA | |
| | GTTCGTCTGGCAGATCCCGTACGGAGCAAACTTGGCG | |
| | TTACGTCAGTTGGGTATCACCCTGTTCTTGGCGGCTA | |
| | TTGGCACTTCCGCGGGAGCTGGGTTTCGCTCAGCTAT | |
| | TAGCGACCCGCAATCTCTGACCATTATTGGATTTGGT | |
| | GCGTTGTTAACCTTGTTTATTAGTATTACCGTCTTGTT | |
| | CGTTGGGCATAAGTTGATGAAAATCCCGTTTGGGGAA | |
| | ACGGCGGGTATCTTAGCTGGAACGCAGACCCATCCA | |
| | GCAGTATTATCATATGTGTCTGACGCATCTCGCAACG | |
| | AGTTGCCAGCCATGGGGTACACCTCAGTGTATCCCTT | |
| | GGCTATGATTGCGAAAATCCTGGCTGCACAAACACTT | |
| | TTGTTTCTGTTGATT | |
| SucE1 | ATGTCCTTCCTGGTCGAGAATCAATTGTTAGCACTTG | SEQ ID |
| | TCGTGATCATGACCGTCGGGCTTTTACTTGGACGTAT | NO: 46 |
| | CAAAATCTTTGGTTTCCGTTTGGGTGTGGCCGCCGTG | |
| | TTGTTCGTCGGCCTTGCTTTAAGCACCATTGAGCCCG | |
| | ACATTTCGGTTCCATCCCTTATTTACGTGGTTGGCCTT | |
| | TCGCTTTTTGTGTATACTATCGGTCTGGAAGCTGGCC | |
| | CCGGTTTTTTTACATCTATGAAGACGACGGGTTTGCG | |
| | CAATAACGCACTGACGTTAGGTGCCATTATCGCGACA | |
| | ACAGCACTTGCGTGGGCACTGATTACCGTCTTGAATA | |
| | TTGATGCCGCCTCAGGAGCTGGTATGCTTACTGGTGC | |
| | CTTAACTAATACGCCCGCTATGGCTGCGGTAGTGGAT | |
| | GCACTTCCCTCATTAATTGATGACACAGGCCAGCTGC | |
| | ATCTTATTGCTGAGCTGCCGGTGGTTGCTTATTCCCTG | |
| | GCTTATCCCTTGGGGGTACTGATTGTGATCTTGAGCA | |
| | TCGCCATCTTTTCTTCAGTGTTTAAGGTTGACCATAAC | |
| | AAGGAGGCAGAAGAGGCTGGGGTAGCGGTCCAAGA | |
| | ACTTAAGGGCCGCCGTATCCGCGTAACTGTAGCTGAC | |
| | TTGCCAGCCCTTGAGAACATTCCTGAGTTGCTTAATT | |
| | TACATGTTATCGTCTCGCGTGTAGAGCGCGACGGAGA | |
| | GCAGTTCATCCCCTTATATGGCGAACATGCACGCATC | |
| | GGCGATGTACTGACTGTCGTGGGGGCCGACGAGGAA | |
| | CTGAACCGCGCGGAAAAAGCCATCGGAGAGTTAATT | |
| | GACGGTGATCCTTACTCTAACGTTGAACTGGACTATC | |
| | GTCGTATCTTCGTCTCTAATACGGCGGTTGTCGGTAC | |
| | ACCCCTGAGCAAATTGCAACCGCTTTTTAAAGATATG | |
| | CTTATTACTCGCATTCGCCGCGGTGATACGGATCTGG | |
| | TAGCTTCCTCGGACATGACGCTTCAATTAGGCGACCG | |
| | CGTTCGTGTGGTTGCCCCAGCCGAGAAACTTCGTGAA | |
| | GCGACTCAGTTGCTTGGAGACTCTTACAAAAAGCTGT | |
| | CCGACTTTAATTTATTGCCTCTTGCTGCGGGCTTAATG | |
| | ATTGGCGTCCTTGTTGGAATGGTTGAATTCCCACTGC | |
| | CTGGGGGGTCATCTTTAAAACTTGGCAATGCCGGTGG | |
| | TCCGTTGGTTGTCGCGCTGTTGCTTGGGATGATCAAT | |
| | CGTACGGGAAAGTTCGTCTGGCAGATCCCGTACGGA | |
| | GCAAACTTGGCGTTACGTCAGTTGGGTATCACCCTGT | |
| | TCTTGGCGGCTATTGGCACTTCCGCGGGAGCTGGGTT | |
| | TCGCTCAGCTATTAGCGACCCGCAATCTCTGACCATT | |
| | ATTGGATTTGGTGCGTTGTTAACCTTGTTTATTAGTAT | |
| | TACCGTCTTGTTCGTTGGGCATAAGTTGATGAAAATC | |
| | CCGTTTGGGGAAACGGCGGGTATCTTAGCTGGAACG | |
| | CAGACCCATCCAGCAGTATTATCATATGTGTCTGACG | |
| | CATCTCGCAACGAGTTGCCAGCCATGGGGTACACCTC | |
| | AGTGTATCCCTTGGCTATGATTGCGAAAATCCTGGCT | |
| | GCACAAACACTTTTGTTTCTGTTGATT | |
| pAraC-dcuC (as | Ttattcacaacctgccctaaactcgctcggactcgccccggtgcattttttaaatactcgc | SEQ ID |
| shown in FIG. | gagaaatagagttgatcgtcaaaaccgacattgcgaccgacggtggcgataggcatcc | NO: 47 |
| 17E; AraC: | gggtggtgctcaaaagcagcttcgcctgactgatgcgctggtcctcgcgccagcttaata | |
| lower case; | cgctaatccctaactgctggcggaacaaatgcgacagacgcgacggcgacaggcaga | |
| pARA: upper | catgctgtgcgacgctggcgatatcaaaattactgtctgccaggtgatcgctgatgtactg | |
| case italics; | acaagcctcgcgtacccgattatccatcggtggatggagcgactcgttaatcgcttccat | |
| RBS: | gcgccgcagtaacaattgctcaagcagatttatcgccagcaattccgaatagcgcccttc | |
| underlined; | cccttgtccggcattaatgatttgcccaaacaggtcgctgaaatgcggctggtgcgcttca | |
| dcuC: bold; FRT | tccgggcgaaagaaaccggtattggcaaatatcgacggccagttaagccattcatgcca | |
| minimal: | gtaggcgcgcggacgaaagtaaacccactggtgataccattcgtgagcctccggatga | |
| underline italics) | cgaccgtagtgatgaatctctccaggcgggaacagcaaaatatcacccggtcggcaga | |
| | caaattctcgtccctgatttttcaccaccccctgaccgcgaatggtgagattgagaatataa | |
| | cctttcattcccagcggtcggtcgataaaaaaatcgagataaccgttggcctcaatcggc | |
| | gttaaacccgccaccagatgggcgttaaacgagtatcccggcagcaggggatcattttg | |
| | cgcttcagccatACTTTTCATACTCCCGCCATTCAGAGAAGAAA | |
| | CCAATTGTCCATATTGCATCAGACATTGCCGTCACTGCGT | |
| | CTTTTACTGGCTCTTCTCGCTAACCCAACCGGTAACCCC | |
| | GCTTATTAAAAGCATTCTGTAACAAAGCGGGACCAAAGC | |
| | CATGACAAAAACGCGTAACAAAAGTGTCTATAATCACGGC | |
| | AGAAAAGTCCACATTGATTATTTGCACGGCGTCACACTTT | |
| | GCTATGCCATAGCATTTTTATCCATAAGATTAGCGGATCC | |
| | AGCCTGACGCTTTTTTTCGCAACTCTCTACTGTTTCTCCA | |
| | TACCCGGGGCCCAATAGGCTCCCTATAAGAGATAGAA | |
| | CTATGCTGACATTCATTGAACTCCTTATTGGGGTT | |
| | GTGGTTATTGTGGGTGTAGCTCGCTACATCATTAA | |
| | AGGGTATTCTGCCACTGGCGTGTTATTTGTCGGTG | |
| | GCCTGTTATTGCTGATTATCAGTGCCATTATGGGG | |
| | CACAAAGTGTTACCGTCCAGCCAGGCTTCAACAG | |
| | GCTACAGCGCCACGGATATCGTTGAATACGTTAAA | |
| | ATATTGCTAATGAGCCGCGGCGGCGACCTCGGCA | |
| | TGATGATTATGATGCTGTGTGGCTTTGCCGCTTAC | |
| | ATGACCCATATCGGCGCGAATGATATGGTGGTCA | |
| | AGCTGGCGTCAAAACCATTGCAGTATATTAACTCC | |
| | CCTTACCTTCTGATGATTGCCGCCTATTTTGTTGC | |
| | CTGTCTGATGTCACTGGCCGTCTCTTCCGCAACCG | |
| | GTCTGGGTGTTTTGCTGATGGCAACCCTGTTTCCG | |
| | GTGATGGTAAACGTTGGTATCAGTCGTGGCGCAG | |
| | CTGCTGCCATTTGTGCCTCCCCGGCGGCGATTATT | |
| | CTCGCACCGACTTCAGGGGATGTGGTGCTGGCGG | |
| | CGCAGGCTTCCGAAATGTCGCTGATTGACTTCGCC | |
| | TTCAAAACAACGCTGCCTATCTCAATTGCTGCAAT | |
| | TATCGGCATGGCGATCGCCCACTTCTTCTGGCAAC | |
| | GTTATCTGGATAAAAAAGAGCACATCTCTCATGAA | |
| | ATGTTAGATGTCAGTGAAATCACCACCACTGCCCC | |
| | TGCGTTTTATGCCATTTTGCCGTTCACGCCGATCA | |
| | TCGGAGTACTGATTTTTGACGGCAAATGGGGTCC | |
| | GCAATTACACATCATCACTATTCTGGTGATTTGTA | |
| | TGCTAATTGCCTCCATTCTGGAGTTCATCCGCAGC | |
| | TTTAATACCCAGAAAGTTTTCTCTGGTCTGGAAGT | |
| | GGCTTATCGCGGTATGGCAGATGCATTTGCTAACG | |
| | TGGTGATGCTGCTGGTTGCCGCTGGGGTATTCGC | |
| | TCAGGGGCTTAGCACCATCGGCTTTATTCAAAGTC | |
| | TGATTTCTATCGCTACCTCGTTTGGTTCGGCGAGT | |
| | ATCATCCTGATGCTGGTATTGGTGATCCTGACAAT | |
| | GCTGGCGGCAGTCACGACCGGTTCAGGCAATGCG | |
| | CCGTTTTATGCGTTTGTTGAGATGATCCCGAAACT | |
| | GGCGCACTCCTCCGGCATTAACCCGGCGTATTTGA | |
| | CTATCCCGATGCTGCAGGCGTCAAACCTGGGTCG | |
| | TACCCTATCACCCGTTTCTGGCGTAGTCGTTGCGG | |
| | TTGCCGGGATGGCGAAGATCTCGCCGTTTGAAGT | |
| | CGTAAAACGCACCTCGGTGCCGGTGCTTGTTGGTT | |
| | TGGTGATTGTTATCGTTGCTACAGAGCTGATGGTG | |
| | CCAGGAACGGCAGCAGCGGTCACAGGCAAGTAAG | |
| | GAATCGACTCCACGTCCCTAGCGTGTGTAGGCTGGAG | |
| | CTGCTTCGAAGTTCCTATACTTTCTAGAGAATAGGAACTTC | |
| dcuC with RBS | GGGCCCAATAGGCTCCCTATAAGAGATAGAACTATG | SEQ ID |
| (underlined) | CTGACATTCATTGAACTCCTTATTGGGGTTGTGGTTAT | NO: 48 |
| | TGTGGGTGTAGCTCGCTACATCATTAAAGGGTATTCT | |
| | GCCACTGGCGTGTTATTTGTCGGTGGCCTGTTATTGCT | |
| | GATTATCAGTGCCATTATGGGGCACAAAGTGTTACCG | |
| | TCCAGCCAGGCTTCAACAGGCTACAGCGCCACGGAT | |
| | ATCGTTGAATACGTTAAAATATTGCTAATGAGCCGCG | |
| | GCGGCGACCTCGGCATGATGATTATGATGCTGTGTGG | |
| | CTTTGCCGCTTACATGACCCATATCGGCGCGAATGAT | |
| | ATGGTGGTCAAGCTGGCGTCAAAACCATTGCAGTATA | |
| | TTAACTCCCCTTACCTTCTGATGATTGCCGCCTATTTT | |
| | GTTGCCTGTCTGATGTCACTGGCCGTCTCTTCCGCAA | |
| | CCGGTCTGGGTGTTTTGCTGATGGCAACCCTGTTTCC | |
| | GGTGATGGTAAACGTTGGTATCAGTCGTGGCGCAGCT | |
| | GCTGCCATTTGTGCCTCCCCGGCGGCGATTATTCTCG | |
| | CACCGACTTCAGGGGATGTGGTGCTGGCGGCGCAGG | |
| | CTTCCGAAATGTCGCTGATTGACTTCGCCTTCAAAAC | |
| | AACGCTGCCTATCTCAATTGCTGCAATTATCGGCATG | |
| | GCGATCGCCCACTTCTTCTGGCAACGTTATCTGGATA | |
| | AAAAAGAGCACATCTCTCATGAAATGTTAGATGTCA | |
| | GTGAAATCACCACCACTGCCCCTGCGTTTTATGCCAT | |
| | TTTGCCGTTCACGCCGATCATCGGAGTACTGATTTTT | |
| | GACGGCAAATGGGGTCCGCAATTACACATCATCACT | |
| | ATTCTGGTGATTTGTATGCTAATTGCCTCCATTCTGGA | |
| | GTTCATCCGCAGCTTTAATACCCAGAAAGTTTTCTCT | |
| | GGTCTGGAAGTGGCTTATCGCGGTATGGCAGATGCAT | |
| | TTGCTAACGTGGTGATGCTGCTGGTTGCCGCTGGGGT | |
| | ATTCGCTCAGGGGCTTAGCACCATCGGCTTTATTCAA | |
| | AGTCTGATTTCTATCGCTACCTCGTTTGGTTCGGCGA | |
| | GTATCATCCTGATGCTGGTATTGGTGATCCTGACAAT | |
| | GCTGGCGGCAGTCACGACCGGTTCAGGCAATGCGCC | |
| | GTTTTATGCGTTTGTTGAGATGATCCCGAAACTGGCG | |
| | CACTCCTCCGGCATTAACCCGGCGTATTTGACTATCC | |
| | CGATGCTGCAGGCGTCAAACCTGGGTCGTACCCTATC | |
| | ACCCGTTTCTGGCGTAGTCGTTGCGGTTGCCGGGATG | |
| | GCGAAGATCTCGCCGTTTGAAGTCGTAAAACGCACCT | |
| | CGGTGCCGGTGCTTGTTGGTTTGGTGATTGTTATCGTT | |
| | GCTACAGAGCTGATGGTGCCAGGAACGGCAGCAGCG | |
| | GTCACAGGCAAGTAA | |
| dcuC | ATGCTGACATTCATTGAACTCCTTATTGGGGTTGTGG | SEQ ID |
| | TTATTGTGGGTGTAGCTCGCTACATCATTAAAGGGTA | NO: 49 |
| | TTCTGCCACTGGCGTGTTATTTGTCGGTGGCCTGTTAT | |
| | TGCTGATTATCAGTGCCATTATGGGGCACAAAGTGTT | |
| | ACCGTCCAGCCAGGCTTCAACAGGCTACAGCGCCAC | |
| | GGATATCGTTGAATACGTTAAAATATTGCTAATGAGC | |
| | CGCGGCGGCGACCTCGGCATGATGATTATGATGCTGT | |
| | GTGGCTTTGCCGCTTACATGACCCATATCGGCGCGAA | |
| | TGATATGGTGGTCAAGCTGGCGTCAAAACCATTGCAG | |
| | TATATTAACTCCCCTTACCTTCTGATGATTGCCGCCTA | |
| | TTTTGTTGCCTGTCTGATGTCACTGGCCGTCTCTTCCG | |
| | CAACCGGTCTGGGTGTTTTGCTGATGGCAACCCTGTT | |
| | TCCGGTGATGGTAAACGTTGGTATCAGTCGTGGCGCA | |
| | GCTGCTGCCATTTGTGCCTCCCCGGCGGCGATTATTC | |
| | TCGCACCGACTTCAGGGGATGTGGTGCTGGCGGCGC | |
| | AGGCTTCCGAAATGTCGCTGATTGACTTCGCCTTCAA | |
| | AACAACGCTGCCTATCTCAATTGCTGCAATTATCGGC | |
| | ATGGCGATCGCCCACTTCTTCTGGCAACGTTATCTGG | |
| | ATAAAAAAGAGCACATCTCTCATGAAATGTTAGATGT | |
| | CAGTGAAATCACCACCACTGCCCCTGCGTTTTATGCC | |
| | ATTTTGCCGTTCACGCCGATCATCGGAGTACTGATTT | |
| | TTGACGGCAAATGGGGTCCGCAATTACACATCATCAC | |
| | TATTCTGGTGATTTGTATGCTAATTGCCTCCATTCTGG | |
| | AGTTCATCCGCAGCTTTAATACCCAGAAAGTTTTCTC | |
| | TGGTCTGGAAGTGGCTTATCGCGGTATGGCAGATGCA | |
| | TTTGCTAACGTGGTGATGCTGCTGGTTGCCGCTGGGG | |
| | TATTCGCTCAGGGGCTTAGCACCATCGGCTTTATTCA | |
| | AAGTCTGATTTCTATCGCTACCTCGTTTGGTTCGGCG | |
| | AGTATCATCCTGATGCTGGTATTGGTGATCCTGACAA | |
| | TGCTGGCGGCAGTCACGACCGGTTCAGGCAATGCGC | |
| | CGTTTTATGCGTTTGTTGAGATGATCCCGAAACTGGC | |
| | GCACTCCTCCGGCATTAACCCGGCGTATTTGACTATC | |
| | CCGATGCTGCAGGCGTCAAACCTGGGTCGTACCCTAT | |
| | CACCCGTTTCTGGCGTAGTCGTTGCGGTTGCCGGGAT | |
| | GGCGAAGATCTCGCCGTTTGAAGTCGTAAAACGCAC | |
| | CTCGGTGCCGGTGCTTGTTGGTTTGGTGATTGTTATCG | |
| | TTGCTACAGAGCTGATGGTGCCAGGAACGGCAGCAG | |
| | CGGTCACAGGCAAGTAA | |
| Para-sucE-dcuC | ttattcacaacctgccctaaactcgctcggactcgccccggtgcattttttaaatactcgcg | SEQ ID |
| construct (as | agaaatagagttgatcgtcaaaaccgacattgcgaccgacggtggcgataggcatccg | NO: 50 |
| shown in FIG. | ggtggtgctcaaaagcagcttcgcctgactgatgcgctggtcctcgcgccagcttaatac | |
| 17F; AraC: | gctaatccctaactgctggcggaacaaatgcgacagacgcgacggcgacaggcagac | |
| lower case; | atgctgtgcgacgctggcgatatcaaaattactgtctgccaggtgatcgctgatgtactga | |
| pARA: upper | caagcctcgcgtacccgattatccatcggtggatggagcgactcgttaatcgcttccatg | |
| case italics; | cgccgcagtaacaattgctcaagcagatttatcgccagcaattccgaatagcgcccttcc | |
| RBS: | ccttgtccggcattaatgatttgcccaaacaggtcgctgaaatgcggctggtgcgcttcat | |
| underlined; | ccgggcgaaagaaaccggtattggcaaatatcgacggccagttaagccattcatgcca | |
| sucE: bold; | gtaggcgcgcggacgaaagtaaacccactggtgataccattcgtgagcctccggatga | |
| dcuC: bold | cgaccgtagtgatgaatctctccaggcgggaacagcaaaatatcacccggtcggcaga | |
| underlined; FRT | caaattctcgtccctgatttttcaccaccccctgaccgcgaatggtgagattgagaatataa | |
| minimal: | cctttcattcccagcggtcggtcgataaaaaaatcgagataaccgttggcctcaatcggc | |
| underline italics) | gttaaacccgccaccagatgggcgttaaacgagtatcccggcagcaggggatcattttg | |
| | cgcttcagccatACTTTTCATACTCCCGCCATTCAGAGAAGAAA | |
| | CCAATTGTCCATATTGCATCAGACATTGCCGTCACTGCGT | |
| | CTTTTACTGGCTCTTCTCGCTAACCCAACCGGTAACCCC | |
| | GCTTATTAAAAGCATTCTGTAACAAAGCGGGACCAAAGC | |
| | CATGACAAAAACGCGTAACAAAAGTGTCTATAATCACGGC | |
| | AGAAAAGTCCACATTGATTATTTGCACGGCGTCACACTTT | |
| | GCTATGCCATAGCATTTTTATCCATAAGATTAGCGGATCC | |
| | AGCCTGACGCTTTTTTTCGCAACTCTCTACTGTTTCTCCA | |
| | TACCCGTTTTTTTGGATGGAGTGAAACGATGTCCTTC | |
| | CTGGTCGAGAATCAATTGTTAGCACTTGTCGTGAT | |
| | CATGACCGTCGGGCTTTTACTTGGACGTATCAAAA | |
| | TCTTTGGTTTCCGTTTGGGTGTGGCCGCCGTGTTG | |
| | TTCGTCGGCCTTGCTTTAAGCACCATTGAGCCCGA | |
| | CATTTCGGTTCCATCCCTTATTTACGTGGTTGGCC | |
| | TTTCGCTTTTTGTGTATACTATCGGTCTGGAAGCT | |
| | GGCCCCGGTTTTTTTACATCTATGAAGACGACGGG | |
| | TTTGCGCAATAACGCACTGACGTTAGGTGCCATTA | |
| | TCGCGACAACAGCACTTGCGTGGGCACTGATTAC | |
| | CGTCTTGAATATTGATGCCGCCTCAGGAGCTGGTA | |
| | TGCTTACTGGTGCCTTAACTAATACGCCCGCTATG | |
| | GCTGCGGTAGTGGATGCACTTCCCTCATTAATTGA | |
| | TGACACAGGCCAGCTGCATCTTATTGCTGAGCTGC | |
| | CGGTGGTTGCTTATTCCCTGGCTTATCCCTTGGGG | |
| | GTACTGATTGTGATCTTGAGCATCGCCATCTTTTC | |
| | TTCAGTGTTTAAGGTTGACCATAACAAGGAGGCAG | |
| | AAGAGGCTGGGGTAGCGGTCCAAGAACTTAAGGG | |
| | CCGCCGTATCCGCGTAACTGTAGCTGACTTGCCAG | |
| | CCCTTGAGAACATTCCTGAGTTGCTTAATTTACAT | |
| | GTTATCGTCTCGCGTGTAGAGCGCGACGGAGAGC | |
| | AGTTCATCCCCTTATATGGCGAACATGCACGCATC | |
| | GGCGATGTACTGACTGTCGTGGGGGCCGACGAGG | |
| | AACTGAACCGCGCGGAAAAAGCCATCGGAGAGTT | |
| | AATTGACGGTGATCCTTACTCTAACGTTGAACTGG | |
| | ACTATCGTCGTATCTTCGTCTCTAATACGGCGGTT | |
| | GTCGGTACACCCCTGAGCAAATTGCAACCGCTTTT | |
| | TAAAGATATGCTTATTACTCGCATTCGCCGCGGTG | |
| | ATACGGATCTGGTAGCTTCCTCGGACATGACGCTT | |
| | CAATTAGGCGACCGCGTTCGTGTGGTTGCCCCAG | |
| | CCGAGAAACTTCGTGAAGCGACTCAGTTGCTTGG | |
| | AGACTCTTACAAAAAGCTGTCCGACTTTAATTTAT | |
| | TGCCTCTTGCTGCGGGCTTAATGATTGGCGTCCTT | |
| | GTTGGAATGGTTGAATTCCCACTGCCTGGGGGGT | |
| | CATCTTTAAAACTTGGCAATGCCGGTGGTCCGTTG | |
| | GTTGTCGCGCTGTTGCTTGGGATGATCAATCGTAC | |
| | GGGAAAGTTCGTCTGGCAGATCCCGTACGGAGCA | |
| | AACTTGGCGTTACGTCAGTTGGGTATCACCCTGTT | |
| | CTTGGCGGCTATTGGCACTTCCGCGGGAGCTGGG | |
| | TTTCGCTCAGCTATTAGCGACCCGCAATCTCTGAC | |
| | CATTATTGGATTTGGTGCGTTGTTAACCTTGTTTA | |
| | TTAGTATTACCGTCTTGTTCGTTGGGCATAAGTTG | |
| | ATGAAAATCCCGTTTGGGGAAACGGCGGGTATCT | |
| | TAGCTGGAACGCAGACCCATCCAGCAGTATTATCA | |
| | TATGTGTCTGACGCATCTCGCAACGAGTTGCCAGC | |
| | CATGGGGTACACCTCAGTGTATCCCTTGGCTATGA | |
| | TTGCGAAAATCCTGGCTGCACAAACACTTTTGTTT | |
| | CTGTTGATTtaatgaGGGCCCAATAGGCTCCCTATAAGA | |
| | GATAGAACTATGCTGACATTCATTGAACTCCTTATT | |
| | GGGGTTGTGGTTATTGTGGGTGTAGCTCGCTACAT | |
| | CATTAAAGGGTATTCTGCCACTGGCGTGTTATTTG | |
| | TCGGTGGCCTGTTATTGCTGATTATCAGTGCCATT | |
| | ATGGGGCACAAAGTGTTACCGTCCAGCCAGGCTT | |
| | CAACAGGCTACAGCGCCACGGATATCGTTGAATA | |
| | CGTTAAAATATTGCTAATGAGCCGCGGCGGCGAC | |
| | CTCGGCATGATGATTATGATGCTGTGTGGCTTTGC | |
| | CGCTTACATGACCCATATCGGCGCGAATGATATGG | |
| | TGGTCAAGCTGGCGTCAAAACCATTGCAGTATATT | |
| | AACTCCCCTTACCTTCTGATGATTGCCGCCTATTT | |
| | TGTTGCCTGTCTGATGTCACTGGCCGTCTCTTCCG | |
| | CAACCGGTCTGGGTGTTTTGCTGATGGCAACCCTG | |
| | TTTCCGGTGATGGTAAACGTTGGTATCAGTCGTGG | |
| | CGCAGCTGCTGCCATTTGTGCCTCCCCGGCGGCG | |
| | ATTATTCTCGCACCGACTTCAGGGGATGTGGTGCT | |
| | GGCGGCGCAGGCTTCCGAAATGTCGCTGATTGAC | |
| | TTCGCCTTCAAAACAACGCTGCCTATCTCAATTGC | |
| | TGCAATTATCGGCATGGCGATCGCCCACTTCTTCT | |
| | GGCAACGTTATCTGGATAAAAAAGAGCACATCTCT | |
| | CATGAAATGTTAGATGTCAGTGAAATCACCACCAC | |
| | TGCCCCTGCGTTTTATGCCATTTTGCCGTTCACGC | |
| | CGATCATCGGAGTACTGATTTTTGACGGCAAATGG | |
| | GGTCCGCAATTACACATCATCACTATTCTGGTGAT | |
| | TTGTATGCTAATTGCCTCCATTCTGGAGTTCATCC | |
| | GCAGCTTTAATACCCAGAAAGTTTTCTCTGGTCTG | |
| | GAAGTGGCTTATCGCGGTATGGCAGATGCATTTG | |
| | CTAACGTGGTGATGCTGCTGGTTGCCGCTGGGGT | |
| | ATTCGCTCAGGGGCTTAGCACCATCGGCTTTATTC | |
| | AAAGTCTGATTTCTATCGCTACCTCGTTTGGTTCG | |
| | GCGAGTATCATCCTGATGCTGGTATTGGTGATCCT | |
| | GACAATGCTGGCGGCAGTCACGACCGGTTCAGGC | |
| | AATGCGCCGTTTTATGCGTTTGTTGAGATGATCCC | |
| | GAAACTGGCGCACTCCTCCGGCATTAACCCGGCG | |
| | TATTTGACTATCCCGATGCTGCAGGCGTCAAACCT | |
| | GGGTCGTACCCTATCACCCGTTTCTGGCGTAGTCG | |
| | TTGCGGTTGCCGGGATGGCGAAGATCTCGCCGTT | |
| | TGAAGTCGTAAAACGCACCTCGGTGCCGGTGCTT | |
| | GTTGGTTTGGTGATTGTTATCGTTGCTACAGAGCT | |
| | GATGGTGCCAGGAACGGCAGCAGCGGTCACAGGC | |
| | AAGTAAGGAATCGACTCCACGTCCCTAGCGTGTGTA | |
| | GGCTGGAGCTGCTTCGAAGTTCCTATACTTTCTAGAGAA | |
| | TAGGAACTTC | |
| SucE1 (bold) | ttattcacaacctgccctaaactcgctcggactcgccccggtgcattttttaaatactcgcg | SEQ ID |
| and dcuC (bold | agaaatagagttgatcgtcaaaaccgacattgcgaccgacggtggcgataggcatccg | NO: 51 |
| underlined) with | ggtggtgctcaaaagcagcttcgcctgactgatgcgctggtcctcgcgccagcttaatac | |
| pAra and RBS | gctaatccctaactgctggcggaacaaatgcgacagacgcgacggcgacaggcagac | |
| (underlined) | atgctgtgcgacgctggcgatatcaaaattactgtctgccaggtgatcgctgatgtactga | |
| | caagcctcgcgtacccgattatccatcggtggatggagcgactcgttaatcgcttccatg | |
| | cgccgcagtaacaattgctcaagcagatttatcgccagcaattccgaatagcgcccttcc | |
| | ccttgtccggcattaatgatttgcccaaacaggtcgctgaaatgcggctggtgcgcttcat | |
| | ccgggcgaaagaaaccggtattggcaaatatcgacggccagttaagccattcatgcca | |
| | gtaggcgcgcggacgaaagtaaacccactggtgataccattcgtgagcctccggatga | |
| | cgaccgtagtgatgaatctctccaggcgggaacagcaaaatatcacccggtcggcaga | |
| | caaattctcgtccctgatttttcaccaccccctgaccgcgaatggtgagattgagaatataa | |
| | cctttcattcccagcggtcggtcgataaaaaaatcgagataaccgttggcctcaatcggc | |
| | gttaaacccgccaccagatgggcgttaaacgagtatcccggcagcaggggatcattttg | |
| | cgcttcagccatACTTTTCATACTCCCGCCATTCAGAGAAGAAA | |
| | CCAATTGTCCATATTGCATCAGACATTGCCGTCACTGCGT | |
| | CTTTTACTGGCTCTTCTCGCTAACCCAACCGGTAACCCC | |
| | GCTTATTAAAAGCATTCTGTAACAAAGCGGGACCAAAGC | |
| | CATGACAAAAACGCGTAACAAAAGTGTCTATAATCACGGC | |
| | AGAAAAGTCCACATTGATTATTTGCACGGCGTCACACTTT | |
| | GCTATGCCATAGCATTTTTATCCATAAGATTAGCGGATCC | |
| | AGCCTGACGCTTTTTTTCGCAACTCTCTACTGTTTCTCCA | |
| | TACCCGTTTTTTTGGATGGAGTGAAACGATGTCCTTC | |
| | CTGGTCGAGAATCAATTGTTAGCACTTGTCGTGAT | |
| | CATGACCGTCGGGCTTTTACTTGGACGTATCAAAA | |
| | TCTTTGGTTTCCGTTTGGGTGTGGCCGCCGTGTTG | |
| | TTCGTCGGCCTTGCTTTAAGCACCATTGAGCCCGA | |
| | CATTTCGGTTCCATCCCTTATTTACGTGGTTGGCC | |
| | TTTCGCTTTTTGTGTATACTATCGGTCTGGAAGCT | |
| | GGCCCCGGTTTTTTTACATCTATGAAGACGACGGG | |
| | TTTGCGCAATAACGCACTGACGTTAGGTGCCATTA | |
| | TCGCGACAACAGCACTTGCGTGGGCACTGATTAC | |
| | CGTCTTGAATATTGATGCCGCCTCAGGAGCTGGTA | |
| | TGCTTACTGGTGCCTTAACTAATACGCCCGCTATG | |
| | GCTGCGGTAGTGGATGCACTTCCCTCATTAATTGA | |
| | TGACACAGGCCAGCTGCATCTTATTGCTGAGCTGC | |
| | CGGTGGTTGCTTATTCCCTGGCTTATCCCTTGGGG | |
| | GTACTGATTGTGATCTTGAGCATCGCCATCTTTTC | |
| | TTCAGTGTTTAAGGTTGACCATAACAAGGAGGCAG | |
| | AAGAGGCTGGGGTAGCGGTCCAAGAACTTAAGGG | |
| | CCGCCGTATCCGCGTAACTGTAGCTGACTTGCCAG | |
| | CCCTTGAGAACATTCCTGAGTTGCTTAATTTACAT | |
| | GTTATCGTCTCGCGTGTAGAGCGCGACGGAGAGC | |
| | AGTTCATCCCCTTATATGGCGAACATGCACGCATC | |
| | GGCGATGTACTGACTGTCGTGGGGGCCGACGAGG | |
| | AACTGAACCGCGCGGAAAAAGCCATCGGAGAGTT | |
| | AATTGACGGTGATCCTTACTCTAACGTTGAACTGG | |
| | ACTATCGTCGTATCTTCGTCTCTAATACGGCGGTT | |
| | GTCGGTACACCCCTGAGCAAATTGCAACCGCTTTT | |
| | TAAAGATATGCTTATTACTCGCATTCGCCGCGGTG | |
| | ATACGGATCTGGTAGCTTCCTCGGACATGACGCTT | |
| | CAATTAGGCGACCGCGTTCGTGTGGTTGCCCCAG | |
| | CCGAGAAACTTCGTGAAGCGACTCAGTTGCTTGG | |
| | AGACTCTTACAAAAAGCTGTCCGACTTTAATTTAT | |
| | TGCCTCTTGCTGCGGGCTTAATGATTGGCGTCCTT | |
| | GTTGGAATGGTTGAATTCCCACTGCCTGGGGGGT | |
| | CATCTTTAAAACTTGGCAATGCCGGTGGTCCGTTG | |
| | GTTGTCGCGCTGTTGCTTGGGATGATCAATCGTAC | |
| | GGGAAAGTTCGTCTGGCAGATCCCGTACGGAGCA | |
| | AACTTGGCGTTACGTCAGTTGGGTATCACCCTGTT | |
| | CTTGGCGGCTATTGGCACTTCCGCGGGAGCTGGG | |
| | TTTCGCTCAGCTATTAGCGACCCGCAATCTCTGAC | |
| | CATTATTGGATTTGGTGCGTTGTTAACCTTGTTTA | |
| | TTAGTATTACCGTCTTGTTCGTTGGGCATAAGTTG | |
| | ATGAAAATCCCGTTTGGGGAAACGGCGGGTATCT | |
| | TAGCTGGAACGCAGACCCATCCAGCAGTATTATCA | |
| | TATGTGTCTGACGCATCTCGCAACGAGTTGCCAGC | |
| | CATGGGGTACACCTCAGTGTATCCCTTGGCTATGA | |
| | TTGCGAAAATCCTGGCTGCACAAACACTTTTGTTT | |
| | CTGTTGATTtaatgaGGGCCCAATAGGCTCCCTATAAGA | |
| | GATAGAACTATGCTGACATTCATTGAACTCCTTATT | |
| | GGGGTTGTGGTTATTGTGGGTGTAGCTCGCTACAT | |
| | CATTAAAGGGTATTCTGCCACTGGCGTGTTATTTG | |
| | TCGGTGGCCTGTTATTGCTGATTATCAGTGCCATT | |
| | ATGGGGCACAAAGTGTTACCGTCCAGCCAGGCTT | |
| | CAACAGGCTACAGCGCCACGGATATCGTTGAATA | |
| | CGTTAAAATATTGCTAATGAGCCGCGGCGGCGAC | |
| | CTCGGCATGATGATTATGATGCTGTGTGGCTTTGC | |
| | CGCTTACATGACCCATATCGGCGCGAATGATATGG | |
| | TGGTCAAGCTGGCGTCAAAACCATTGCAGTATATT | |
| | AACTCCCCTTACCTTCTGATGATTGCCGCCTATTT | |
| | TGTTGCCTGTCTGATGTCACTGGCCGTCTCTTCCG | |
| | CAACCGGTCTGGGTGTTTTGCTGATGGCAACCCTG | |
| | TTTCCGGTGATGGTAAACGTTGGTATCAGTCGTGG | |
| | CGCAGCTGCTGCCATTTGTGCCTCCCCGGCGGCG | |
| | ATTATTCTCGCACCGACTTCAGGGGATGTGGTGCT | |
| | GGCGGCGCAGGCTTCCGAAATGTCGCTGATTGAC | |
| | TTCGCCTTCAAAACAACGCTGCCTATCTCAATTGC | |
| | TGCAATTATCGGCATGGCGATCGCCCACTTCTTCT | |
| | GGCAACGTTATCTGGATAAAAAAGAGCACATCTCT | |
| | CATGAAATGTTAGATGTCAGTGAAATCACCACCAC | |
| | TGCCCCTGCGTTTTATGCCATTTTGCCGTTCACGC | |
| | CGATCATCGGAGTACTGATTTTTGACGGCAAATGG | |
| | GGTCCGCAATTACACATCATCACTATTCTGGTGAT | |
| | TTGTATGCTAATTGCCTCCATTCTGGAGTTCATCC | |
| | GCAGCTTTAATACCCAGAAAGTTTTCTCTGGTCTG | |
| | GAAGTGGCTTATCGCGGTATGGCAGATGCATTTG | |
| | CTAACGTGGTGATGCTGCTGGTTGCCGCTGGGGT | |
| | ATTCGCTCAGGGGCTTAGCACCATCGGCTTTATTC | |
| | AAAGTCTGATTTCTATCGCTACCTCGTTTGGTTCG | |
| | GCGAGTATCATCCTGATGCTGGTATTGGTGATCCT | |
| | GACAATGCTGGCGGCAGTCACGACCGGTTCAGGC | |
| | AATGCGCCGTTTTATGCGTTTGTTGAGATGATCCC | |
| | GAAACTGGCGCACTCCTCCGGCATTAACCCGGCG | |
| | TATTTGACTATCCCGATGCTGCAGGCGTCAAACCT | |
| | GGGTCGTACCCTATCACCCGTTTCTGGCGTAGTCG | |
| | TTGCGGTTGCCGGGATGGCGAAGATCTCGCCGTT | |
| | TGAAGTCGTAAAACGCACCTCGGTGCCGGTGCTT | |
| | GTTGGTTTGGTGATTGTTATCGTTGCTACAGAGCT | |
| | GATGGTGCCAGGAACGGCAGCAGCGGTCACAGGC | |
| | AAGTAA | |
| SucE1 (bold) | CCCGTTTTTTTGGATGGAGTGAAACGATGTCCTTCCT | SEQ ID |
| and dcuC (bold | GGTCGAGAATCAATTGTTAGCACTTGTCGTGATCA | NO: 52 |
| underlined) with | TGACCGTCGGGCTTTTACTTGGACGTATCAAAATC | |
| RBS | TTTGGTTTCCGTTTGGGTGTGGCCGCCGTGTTGTT | |
| (underlined) | CGTCGGCCTTGCTTTAAGCACCATTGAGCCCGACA | |
| | TTTCGGTTCCATCCCTTATTTACGTGGTTGGCCTT | |
| | TCGCTTTTTGTGTATACTATCGGTCTGGAAGCTGG | |
| | CCCCGGTTTTTTTACATCTATGAAGACGACGGGTT | |
| | TGCGCAATAACGCACTGACGTTAGGTGCCATTATC | |
| | GCGACAACAGCACTTGCGTGGGCACTGATTACCG | |
| | TCTTGAATATTGATGCCGCCTCAGGAGCTGGTATG | |
| | CTTACTGGTGCCTTAACTAATACGCCCGCTATGGC | |
| | TGCGGTAGTGGATGCACTTCCCTCATTAATTGATG | |
| | ACACAGGCCAGCTGCATCTTATTGCTGAGCTGCCG | |
| | GTGGTTGCTTATTCCCTGGCTTATCCCTTGGGGGT | |
| | ACTGATTGTGATCTTGAGCATCGCCATCTTTTCTT | |
| | CAGTGTTTAAGGTTGACCATAACAAGGAGGCAGA | |
| | AGAGGCTGGGGTAGCGGTCCAAGAACTTAAGGGC | |
| | CGCCGTATCCGCGTAACTGTAGCTGACTTGCCAGC | |
| | CCTTGAGAACATTCCTGAGTTGCTTAATTTACATG | |
| | TTATCGTCTCGCGTGTAGAGCGCGACGGAGAGCA | |
| | GTTCATCCCCTTATATGGCGAACATGCACGCATCG | |
| | GCGATGTACTGACTGTCGTGGGGGCCGACGAGGA | |
| | ACTGAACCGCGCGGAAAAAGCCATCGGAGAGTTA | |
| | ATTGACGGTGATCCTTACTCTAACGTTGAACTGGA | |
| | CTATCGTCGTATCTTCGTCTCTAATACGGCGGTTG | |
| | TCGGTACACCCCTGAGCAAATTGCAACCGCTTTTT | |
| | AAAGATATGCTTATTACTCGCATTCGCCGCGGTGA | |
| | TACGGATCTGGTAGCTTCCTCGGACATGACGCTTC | |
| | AATTAGGCGACCGCGTTCGTGTGGTTGCCCCAGC | |
| | CGAGAAACTTCGTGAAGCGACTCAGTTGCTTGGA | |
| | GACTCTTACAAAAAGCTGTCCGACTTTAATTTATT | |
| | GCCTCTTGCTGCGGGCTTAATGATTGGCGTCCTTG | |
| | TTGGAATGGTTGAATTCCCACTGCCTGGGGGGTC | |
| | ATCTTTAAAACTTGGCAATGCCGGTGGTCCGTTGG | |
| | TTGTCGCGCTGTTGCTTGGGATGATCAATCGTACG | |
| | GGAAAGTTCGTCTGGCAGATCCCGTACGGAGCAA | |
| | ACTTGGCGTTACGTCAGTTGGGTATCACCCTGTTC | |
| | TTGGCGGCTATTGGCACTTCCGCGGGAGCTGGGT | |
| | TTCGCTCAGCTATTAGCGACCCGCAATCTCTGACC | |
| | ATTATTGGATTTGGTGCGTTGTTAACCTTGTTTAT | |
| | TAGTATTACCGTCTTGTTCGTTGGGCATAAGTTGA | |
| | TGAAAATCCCGTTTGGGGAAACGGCGGGTATCTT | |
| | AGCTGGAACGCAGACCCATCCAGCAGTATTATCAT | |
| | ATGTGTCTGACGCATCTCGCAACGAGTTGCCAGCC | |
| | ATGGGGTACACCTCAGTGTATCCCTTGGCTATGAT | |
| | TGCGAAAATCCTGGCTGCACAAACACTTTTGTTTC | |
| | TGTTGATTtaatgaGGGCCCAATAGGCTCCCTATAAGAG | |
| | ATAGAACTATGCTGACATTCATTGAACTCCTTATTG | |
| | GGGTTGTGGTTATTGTGGGTGTAGCTCGCTACATC | |
| | ATTAAAGGGTATTCTGCCACTGGCGTGTTATTTGT | |
| | CGGTGGCCTGTTATTGCTGATTATCAGTGCCATTA | |
| | TGGGGCACAAAGTGTTACCGTCCAGCCAGGCTTC | |
| | AACAGGCTACAGCGCCACGGATATCGTTGAATAC | |
| | GTTAAAATATTGCTAATGAGCCGCGGCGGCGACC | |
| | TCGGCATGATGATTATGATGCTGTGTGGCTTTGCC | |
| | GCTTACATGACCCATATCGGCGCGAATGATATGGT | |
| | GGTCAAGCTGGCGTCAAAACCATTGCAGTATATTA | |
| | ACTCCCCTTACCTTCTGATGATTGCCGCCTATTTT | |
| | GTTGCCTGTCTGATGTCACTGGCCGTCTCTTCCGC | |
| | AACCGGTCTGGGTGTTTTGCTGATGGCAACCCTGT | |
| | TTCCGGTGATGGTAAACGTTGGTATCAGTCGTGGC | |
| | GCAGCTGCTGCCATTTGTGCCTCCCCGGCGGCGA | |
| | TTATTCTCGCACCGACTTCAGGGGATGTGGTGCTG | |
| | GCGGCGCAGGCTTCCGAAATGTCGCTGATTGACT | |
| | TCGCCTTCAAAACAACGCTGCCTATCTCAATTGCT | |
| | GCAATTATCGGCATGGCGATCGCCCACTTCTTCTG | |
| | GCAACGTTATCTGGATAAAAAAGAGCACATCTCTC | |
| | ATGAAATGTTAGATGTCAGTGAAATCACCACCACT | |
| | GCCCCTGCGTTTTATGCCATTTTGCCGTTCACGCC | |
| | GATCATCGGAGTACTGATTTTTGACGGCAAATGGG | |
| | GTCCGCAATTACACATCATCACTATTCTGGTGATT | |
| | TGTATGCTAATTGCCTCCATTCTGGAGTTCATCCG | |
| | CAGCTTTAATACCCAGAAAGTTTTCTCTGGTCTGG | |
| | AAGTGGCTTATCGCGGTATGGCAGATGCATTTGCT | |
| | AACGTGGTGATGCTGCTGGTTGCCGCTGGGGTAT | |
| | TCGCTCAGGGGCTTAGCACCATCGGCTTTATTCAA | |
| | AGTCTGATTTCTATCGCTACCTCGTTTGGTTCGGC | |
| | GAGTATCATCCTGATGCTGGTATTGGTGATCCTGA | |
| | CAATGCTGGCGGCAGTCACGACCGGTTCAGGCAA | |
| | TGCGCCGTTTTATGCGTTTGTTGAGATGATCCCGA | |
| | AACTGGCGCACTCCTCCGGCATTAACCCGGCGTAT | |
| | TTGACTATCCCGATGCTGCAGGCGTCAAACCTGG | |
| | GTCGTACCCTATCACCCGTTTCTGGCGTAGTCGTT | |
| | GCGGTTGCCGGGATGGCGAAGATCTCGCCGTTTG | |
| | AAGTCGTAAAACGCACCTCGGTGCCGGTGCTTGTT | |
| | GGTTTGGTGATTGTTATCGTTGCTACAGAGCTGAT | |
| | GGTGCCAGGAACGGCAGCAGCGGTCACAGGCAAG | |
| | TAA | |

### Example 14 Activity of the 2-Methyl-Citrate Pathway

To determine the suitability of 2-methyl citrate pathway for propionate consumption by the genetically engineered bacteria, a circuit in which the prpB, prpC, prpD, and prpE genes are expressed under the control of an inducible promoter is generated. 2-methyl citrate pathway sequences are shown in Table 33.

TABLE 33
Methyl Citrate Pathway Circuit Sequences

| | | SEQ ID |
|---|---|---|
| Description | Sequence | NO |
| Construct comprising | ttaagacccactttcacatttaagttgtttttctaatccgcatatgatcaattcaag | SEQ ID |
| TetR (reverse | gccgaataagaaggctggctctgcaccttggtgatcaaataattcgatagctt | NO: 53 |
| orientation, | gtcgtaataatggcggcatactatcagtagtaggtgtttccctttcttctttagcg | |
| lowercase) and a | acttgatgctcttgatcttccaatacgcaacctaaagtaaaatgccccacagcg | |
| prpBCDE gene | ctgagtgcatataatgcattctctagtgaaaaaccttgttggcataaaaaggct | |
| cassette under the | aattgattttcgagagtttcatactgtttttctgtaggccgtgtacctaaatgtactt | |
| Ptet promoter (italics) | ttgctccatcgcgatgacttagtaaagcacatctaaaacttttagcgttattacgt | |
| (as shown in FIG. | aaaaaatcttgccagctttccccttctaaagggcaaaagtgagtatggtgccta | |
| 20); ribosome | tctaacatctcaatggctaaggcgtcgagcaaagcccgcttattttttacatgcc | |
| binding sites are | aatacaatgtaggctgctctacacctagcttctgggcgagtttacgggttgtta | |
| underlined; coding | aaccttcgattccgacctcattaagcagctctaatgcgctgttaatcactttactt | |
| regions in bold | ttatctaatctagacatcatTAATTCCTAATTTTTGTTGACACT | |
| underline | CTATCATTGATAGAGTTATTTTACCACTCCCTATCA | |
| | GTGATAGAGAAAAGTGAAATGTCTCTACACTCT | |
| | CCAGGTAAAGCGTTTCGCGCTGCACTTAGC | |
| | AAAGAAACCCCGTTGCAAATTGTTGGCACC | |
| | ATCAACGCTAACCATGCGCTGCTGGCGCAG | |
| | CGTGCCGGATATCAGGCGATTTATCTCTCCG | |
| | GCGGTGGCGTGGCGGCAGGATCGCTGGGG | |
| | CTGCCCGATCTCGGTATTTCTACTCTTGATG | |
| | ACGTGCTGACAGATATTCGCCGTATCACCG | |
| | ACGTTTGTTCGCTGCCGCTGCTGGTGGATG | |
| | CGGATATCGGTTTTGGTTCTTCAGCCTTTAA | |
| | CGTGGCGCGTACGGTGAAATCAATGATTAA | |
| | AGCCGGTGCGGCAGGATTGCATATTGAAGA | |
| | TCAGGTTGGTGCGAAACGCTGCGGTCATCG | |
| | TCCGAATAAAGCGATCGTCTCGAAAGAAGA | |
| | GATGGTGGATCGGATCCGCGCGGCGGTGGA | |
| | TGCGAAAACCGATCCTGATTTTGTGATCATG | |
| | GCGCGCACCGATGCGCTGGCGGTAGAGGGG | |
| | CTGGATGCGGCGATCGAGCGTGCGCAGGCC | |
| | TATGTTGAAGCGGGTGCCGAAATGCTGTTC | |
| | CCGGAGGCGATTACCGAACTCGCCATGTAT | |
| | CGCCAGTTTGCCGATGCGGTGCAGGTGCCG | |
| | ATCCTCTCCAACATTACCGAATTTGGCGCAA | |
| | CACCGCTGTTTACCACCGACGAATTACGCA | |
| | GCGCCCATGTCGCAATGGCGCTCTACCCGC | |
| | TTTCAGCGTTTCGCGCCATGAACCGCGCCG | |
| | CTGAACATGTCTATAACATCCTGCGTCAGGA | |
| | AGGCACACAGAAAAGCGTCATCGACACCAT | |
| | GCAGACCCGCAACGAGCTGTACGAAAGCAT | |
| | CAACTACTACCAGTACGAAGAGAAGCTCGA | |
| | CGACCTGTTTGCCCGTGGTCAGGTGAAATAA | |
| | AAACGCCCGTTGGTTGTATTCGACAACCGATG | |
| | CCTGATGCGCCGCTGACGCGACTTATCAGGCC | |
| | TACGAGGTGAACTGAACTGTAGGTCGGATAAG | |
| | ACGCATAGCGTCGCATCCGACAACAATCTCGA | |
| | CCCTACAAATGATAACAATGACGAGGACAATA | |
| | TGAGCGACACAACGATCCTGCAAAACAGTA | |
| | CCCATGTCATTAAACCGAAAAAATCGGTGG | |
| | CACTTTCCGGCGTTCCGGCGGGCAATACGG | |
| | CGCTCTGCACCGTGGGTAAAAGCGGCAACG | |
| | ACCTGCATTACCGTGGCTACGATATTCTTGA | |
| | TCTGGCGGAACATTGTGAATTTGAAGAAGT | |
| | GGCGCACCTGCTGATCCACGGCAAACTGCC | |
| | AACCCGTGACGAACTCGCCGCCTACAAAAC | |
| | GAAACTGAAAGCCCTGCGTGGTTTACCGGC | |
| | TAACGTGCGTACCGTGCTGGAAGCCTTACC | |
| | GGCGGCGTCACACCCGATGGATGTTATGCG | |
| | CACCGGCGTTTCCGCGCTCGGCTGCACGCT | |
| | GCCAGAAAAAGAGGGGCACACCGTTTCTGG | |
| | TGCGCGGGATATTGCCGACAAACTGCTGGC | |
| | GTCACTTAGTTCGATTCTTCTCTACTGGTAT | |
| | CACTACAGCCACAACGGCGAACGCATCCAG | |
| | CCGGAAACTGATGACGACTCTATCGGCGGT | |
| | CACTTCCTGCATCTGCTGCACGGCGAAAAG | |
| | CCGTCGCAAAGCTGGGAAAAGGCGATGCAT | |
| | ATCTCGCTGGTGCTGTACGCCGAACACGAG | |
| | TTTAACGCTTCCACCTTTACCAGCCGGGTGA | |
| | TTGCGGGCACTGGCTCTGATATGTATTCCGC | |
| | CATTATTGGCGCGATTGGCGCACTGCGCGG | |
| | GCCGAAACACGGCGGGGCGAATGAAGTGTC | |
| | GCTGGAGATCCAGCAACGCTACGAAACGCC | |
| | GGGCGAAGCCGAAGCCGATATCCGCAAGCG | |
| | GGTGGAAAACAAAGAAGTGGTCATTGGTTT | |
| | TGGGCATCCGGTTTATACCATCGCCGACCC | |
| | GCGTCATCAGGTGATCAAACGTGTGGCGAA | |
| | GCAGCTCTCGCAGGAAGGCGGCTCGCTGAA | |
| | GATGTACAACATCGCCGATCGCCTGGAAAC | |
| | GGTGATGTGGGAGAGCAAAAAGATGTTCCC | |
| | CAATCTCGACTGGTTCTCCGCTGTTTCCTAC | |
| | AACATGATGGGTGTTCCCACCGAGATGTTC | |
| | ACACCACTGTTTGTTATCGCCCGCGTCACTG | |
| | GCTGGGCGGCGCACATTATCGAACAACGTC | |
| | AGGACAACAAAATTATCCGTCCTTCCGCCAA | |
| | TTATGTTGGACCGGAAGACCGCCAGTTTGT | |
| | CGCGCTGGATAAGCGCCAGTAA | |
| | ACCTCTACGAATAACAATAAGGAAACGTACCC | |
| | AATGTCAGCTCAAATCAACAACATCCGCCCG | |
| | GAATTTGATCGTGAAATCGTTGATATCGTCG | |
| | ATTACGTGATGAACTACGAAATCAGCTCCAG | |
| | AGTAGCCTACGACACCGCTCATTACTGCCTG | |
| | CTTGACACGCTCGGCTGCGGTCTGGAAGCT | |
| | CTCGAATATCCGGCCTGTAAAAAACTGCTG | |
| | GGGCCAATTGTCCCCGGCACCGTCGTACCC | |
| | AACGGCGTGCGCGTTCCCGGAACTCAGTTT | |
| | CAGCTCGACCCCGTCCAGGCGGCATTTAAC | |
| | ATTGGCGCGATGATCCGTTGGCTCGATTTCA | |
| | ACGATACCTGGCTGGCGGCGGAGTGGGGGC | |
| | ATCCTTCCGACAACCTCGGCGGCATTCTGG | |
| | CAACGGCGGACTGGCTTTCGCGCAACGCGA | |
| | TCGCCAGCGGCAAAGCGCCGTTGACCATGA | |
| | AACAGGTGCTGACCGGAATGATCAAAGCCC | |
| | ATGAAATTCAGGGCTGCATCGCGCTGGAAA | |
| | ACTCCTTTAACCGCGTTGGTCTCGACCACGT | |
| | TCTGTTAGTGAAAGTGGCTTCCACCGCCGT | |
| | GGTCGCCGAAATGCTCGGCCTGACCCGCGA | |
| | GGAAATTCTCAACGCCGTTTCGCTGGCATG | |
| | GGTAGACGGACAGTCGCTGCGCACTTATCG | |
| | TCATGCACCGAACACCGGTACGCGTAAATC | |
| | CTGGGCGGCGGGCGATGCTACATCCCGCGC | |
| | GGTACGTCTGGCGCTGATGGCGAAAACGGG | |
| | CGAAATGGGTTACCCGTCAGCCCTGACCGC | |
| | GCCGGTGTGGGGTTTCTACGACGTCTCCTTT | |
| | AAAGGTGAGTCATTCCGCTTCCAGCGTCCG | |
| | TACGGTTCCTACGTCATGGAAAATGTGCTGT | |
| | TCAAAATCTCCTTCCCGGCGGAGTTCCACTC | |
| | CCAGACGGCAGTTGAAGCGGCGATGACGCT | |
| | CTATGAACAGATGCAGGCAGCAGGCAAAAC | |
| | GGCGGCAGATATCGAAAAAGTGACCATTCG | |
| | CACCCACGAAGCCTGTATTCGCATCATCGAC | |
| | AAAAAAGGGCCGCTCAATAACCCGGCAGAC | |
| | CGCGACCACTGCATTCAGTACATGGTGGCG | |
| | ATCCCGCTGCTGTTCGGACGCTTAACGGCG | |
| | GCAGATTACGAGGACAACGTTGCGCAAGAT | |
| | AAACGCATCGACGCCCTGCGCGAGAAGATC | |
| | AATTGCTTTGAAGATCCGGCGTTTACCGCTG | |
| | ACTACCACGACCCGGAAAAACGCGCCATCG | |
| | CCAATGCCATAACCCTTGAGTTCACCGACG | |
| | GCACACGATTTGAAGAAGTGGTGGTGGAGT | |
| | ACCCAATTGGTCATGCTCGCCGCCGTCAGG | |
| | ATGGCATTCCGAAGCTGGTCGATAAATTCAA | |
| | AATCAATCTCGCGCGCCAGTTCCCGACTCG | |
| | CCAGCAGCAGCGCATTCTGGAGGTTTCTCT | |
| | CGACAGAACTCGCCTGGAACAGATGCCGGT | |
| | CAATGAGTATCTCGACCTGTACGTCATTTAA | |
| | GTAAACGGCGGTAAGGCGTAAGTTCAACAGGA | |
| | GAGCATTATGTCTTTTAGCGAATTTTATCAG | |
| | CGTTCGATTAACGAACCGGAGAAGTTCTGG | |
| | GCCGAGCAGGCCCGGCGTATTGACTGGCAG | |
| | ACGCCCTTTACGCAAACGCTCGACCACAGC | |
| | AACCCGCCGTTTGCCCGTTGGTTTTGTGAAG | |
| | GCCGAACCAACTTGTGTCACAACGCTATCG | |
| | ACCGCTGGCTGGAGAAACAGCCAGAGGCGC | |
| | TGGCATTGATTGCCGTCTCTTCGGAAACAGA | |
| | GGAAGAGCGTACCTTTACCTTCCGCCAGTTA | |
| | CATGACGAAGTGAATGCGGTGGCGTCAATG | |
| | CTGCGCTCACTGGGCGTGCAGCGTGGCGAT | |
| | CGGGTGCTGGTGTATATGCCGATGATTGCC | |
| | GAAGCGCATATTACCCTGCTGGCCTGCGCG | |
| | CGCATTGGTGCTATTCACTCGGTGGTGTTTG | |
| | GGGGATTTGCTTCGCACAGCGTGGCAACGC | |
| | GAATTGATGACGCTAAACCGGTGCTGATTG | |
| | TCTCGGCTGATGCCGGGGCGCGCGGCGGTA | |
| | AAATCATTCCGTATAAAAAATTGCTCGACGA | |
| | TGCGATAAGTCAGGCACAGCATCAGCCGCG | |
| | TCACGTTTTACTGGTGGATCGCGGGCTGGC | |
| | GAAAATGGCGCGCGTTAGCGGGCGGGATGT | |
| | CGATTTCGCGTCGTTGCGCCATCAACACATC | |
| | GGCGCGCGGGTGCCGGTGGCATGGCTGGAA | |
| | TCCAACGAAACCTCCTGCATTCTCTACACCT | |
| | CCGGCACGACCGGCAAACCTAAAGGTGTGC | |
| | AGCGTGATGTCGGCGGATATGCGGTGGCGC | |
| | TGGCGACCTCGATGGACACCATTTTTGGCG | |
| | GCAAAGCGGGCGGCGTGTTCTTTTGTGCTT | |
| | CGGATATCGGCTGGGTGGTAGGGCATTCGT | |
| | ATATCGTTTACGCGCCGCTGCTGGCGGGGA | |
| | TGGCGACTATCGTTTACGAAGGATTGCCGA | |
| | CCTGGCCGGACTGCGGCGTGTGGTGGAAAA | |
| | TTGTCGAGAAATATCAGGTTAGCCGCATGTT | |
| | CTCAGCGCCGACCGCCATTCGCGTGCTGAA | |
| | AAAATTCCCTACCGCTGAAATTCGCAAACAC | |
| | GATCTTTCGTCGCTGGAAGTGCTCTATCTGG | |
| | CTGGAGAACCGCTGGACGAGCCGACCGCCA | |
| | GTTGGGTGAGCAATACGCTGGATGTGCCGG | |
| | TCATCGACAACTACTGGCAGACCGAATCCG | |
| | GCTGGCCGATTATGGCGATTGCTCGCGGTC | |
| | TGGATGACAGACCGACGCGTCTGGGAAGCC | |
| | CCGGCGTGCCGATGTATGGCTATAACGTGC | |
| | AGTTGCTCAATGAAGTCACCGGCGAACCGT | |
| | GTGGCGTCAATGAGAAAGGGATGCTGGTAG | |
| | TGGAGGGGCCATTGCCGCCAGGCTGTATTC | |
| | AAACCATCTGGGGCGACGACGACCGCTTTG | |
| | TGAAGACGTACTGGTCGCTGTTTTCCCGTCC | |
| | GGTGTACGCCACTTTTGACTGGGGCATCCG | |
| | CGATGCTGACGGTTATCACTTTATTCTCGGG | |
| | CGCACTGACGATGTGATTAACGTTGCCGGA | |
| | CATCGGCTGGGTACGCGTGAGATTGAAGAG | |
| | AGTATCTCCAGTCATCCGGGCGTTGCCGAA | |
| | GTGGCGGTGGTTGGGGTGAAAGATGCGCTG | |
| | AAAGGGCAGGTGGCGGTGGCGTTTGTCATT | |
| | CCGAAAGAGAGCGACAGTCTGGAAGACCGT | |
| | GAGGTGGCGCACTCGCAAGAGAAGGCGATT | |
| | ATGGCGCTGGTGGACAGCCAGATTGGCAAC | |
| | TTTGGCCGCCCGGCGCACGTCTGGTTTGTC | |
| | TCGCAATTGCCAAAAACGCGATCCGGAAAA | |
| | ATGCTGCGCCGCACGATCCAGGCGATTTGC | |
| | GAAGGACGCGATCCTGGGGATCTGACGACC | |
| | ATTGATGATCCGGCGTCGTTGGATCAGATC | |
| | CGCCAGGCGATGGAAGAGTAGGTCGGATAA | |
| | GGCGCTCGCGCCGCATCCGACACCGTGCGCAG | |
| | ATGCCTGATGCGACGCTGACGCGTCTTATCATG | |
| | CCTCGCTCTCGAGTCCCGTCAAGTCAGCGTAAT | |
| | GCTCTGCCAGTGTTACAACCAATTAACCAATTC | |
| | TGAT | |
| Construct comprising | TAATTCCTAATTTTTGTTGACACTCTATCATTGATA | SEQ ID |
| a prpBCDE gene | GAGTTATTTTACCACTCCCTATCAGTGATAGAGAA | NO: 54 |
| cassette under the | AAGTGAA | |
| control of the Ptet | ATGTCTCTACACTCTCCAGGTAAAGCGTTTC | |
| promoter (italics) (as | GCGCTGCACTTAGCAAAGAAACCCCGTTGC | |
| shown in FIG. 20) | AAATTGTTGGCACCATCAACGCTAACCATGC | |
| ribosome binding | GCTGCTGGCGCAGCGTGCCGGATATCAGGC | |
| sites are underlined; | GATTTATCTCTCCGGCGGTGGCGTGGCGGC | |
| coding regions in | AGGATCGCTGGGGCTGCCCGATCTCGGTAT | |
| bold underlined;. | TTCTACTCTTGATGACGTGCTGACAGATATT | |
| | CGCCGTATCACCGACGTTTGTTCGCTGCCG | |
| | CTGCTGGTGGATGCGGATATCGGTTTTGGT | |
| | TCTTCAGCCTTTAACGTGGCGCGTACGGTG | |
| | AAATCAATGATTAAAGCCGGTGCGGCAGGA | |
| | TTGCATATTGAAGATCAGGTTGGTGCGAAA | |
| | CGCTGCGGTCATCGTCCGAATAAAGCGATC | |
| | GTCTCGAAAGAAGAGATGGTGGATCGGATC | |
| | CGCGCGGCGGTGGATGCGAAAACCGATCCT | |
| | GATTTTGTGATCATGGCGCGCACCGATGCG | |
| | CTGGCGGTAGAGGGGCTGGATGCGGCGATC | |
| | GAGCGTGCGCAGGCCTATGTTGAAGCGGGT | |
| | GCCGAAATGCTGTTCCCGGAGGCGATTACC | |
| | GAACTCGCCATGTATCGCCAGTTTGCCGAT | |
| | GCGGTGCAGGTGCCGATCCTCTCCAACATT | |
| | ACCGAATTTGGCGCAACACCGCTGTTTACCA | |
| | CCGACGAATTACGCAGCGCCCATGTCGCAA | |
| | TGGCGCTCTACCCGCTTTCAGCGTTTCGCGC | |
| | CATGAACCGCGCCGCTGAACATGTCTATAA | |
| | CATCCTGCGTCAGGAAGGCACACAGAAAAG | |
| | CGTCATCGACACCATGCAGACCCGCAACGA | |
| | GCTGTACGAAAGCATCAACTACTACCAGTAC | |
| | GAAGAGAAGCTCGACGACCTGTTTGCCCGT | |
| | GGTCAGGTGAAATAA | |
| | AAACGCCCGTTGGTTGTATTCGACAACCGATG | |
| | CCTGATGCGCCGCTGACGCGACTTATCAGGCC | |
| | TACGAGGTGAACTGAACTGTAGGTCGGATAAG | |
| | ACGCATAGCGTCGCATCCGACAACAATCTCGA | |
| | CCCTACAAATGATAACAATGACGAGGACAATA | |
| | TGAGCGACACAACGATCCTGCAAAACAGTA | |
| | CCCATGTCATTAAACCGAAAAAATCGGTGG | |
| | CACTTTCCGGCGTTCCGGCGGGCAATACGG | |
| | CGCTCTGCACCGTGGGTAAAAGCGGCAACG | |
| | ACCTGCATTACCGTGGCTACGATATTCTTGA | |
| | TCTGGCGGAACATTGTGAATTTGAAGAAGT | |
| | GGCGCACCTGCTGATCCACGGCAAACTGCC | |
| | AACCCGTGACGAACTCGCCGCCTACAAAAC | |
| | GAAACTGAAAGCCCTGCGTGGTTTACCGGC | |
| | TAACGTGCGTACCGTGCTGGAAGCCTTACC | |
| | GGCGGCGTCACACCCGATGGATGTTATGCG | |
| | CACCGGCGTTTCCGCGCTCGGCTGCACGCT | |
| | GCCAGAAAAAGAGGGGCACACCGTTTCTGG | |
| | TGCGCGGGATATTGCCGACAAACTGCTGGC | |
| | GTCACTTAGTTCGATTCTTCTCTACTGGTAT | |
| | CACTACAGCCACAACGGCGAACGCATCCAG | |
| | CCGGAAACTGATGACGACTCTATCGGCGGT | |
| | CACTTCCTGCATCTGCTGCACGGCGAAAAG | |
| | CCGTCGCAAAGCTGGGAAAAGGCGATGCAT | |
| | ATCTCGCTGGTGCTGTACGCCGAACACGAG | |
| | TTTAACGCTTCCACCTTTACCAGCCGGGTGA | |
| | TTGCGGGCACTGGCTCTGATATGTATTCCGC | |
| | CATTATTGGCGCGATTGGCGCACTGCGCGG | |
| | GCCGAAACACGGCGGGGCGAATGAAGTGTC | |
| | GCTGGAGATCCAGCAACGCTACGAAACGCC | |
| | GGGCGAAGCCGAAGCCGATATCCGCAAGCG | |
| | GGTGGAAAACAAAGAAGTGGTCATTGGTTT | |
| | TGGGCATCCGGTTTATACCATCGCCGACCC | |
| | GCGTCATCAGGTGATCAAACGTGTGGCGAA | |
| | GCAGCTCTCGCAGGAAGGCGGCTCGCTGAA | |
| | GATGTACAACATCGCCGATCGCCTGGAAAC | |
| | GGTGATGTGGGAGAGCAAAAAGATGTTCCC | |
| | CAATCTCGACTGGTTCTCCGCTGTTTCCTAC | |
| | AACATGATGGGTGTTCCCACCGAGATGTTC | |
| | ACACCACTGTTTGTTATCGCCCGCGTCACTG | |
| | GCTGGGCGGCGCACATTATCGAACAACGTC | |
| | AGGACAACAAAATTATCCGTCCTTCCGCCAA | |
| | TTATGTTGGACCGGAAGACCGCCAGTTTGT | |
| | CGCGCTGGATAAGCGCCAGTAA | |
| | ACCTCTACGAATAACAATAAGGAAACGTACCC | |
| | AATGTCAGCTCAAATCAACAACATCCGCCCG | |
| | GAATTTGATCGTGAAATCGTTGATATCGTCG | |
| | ATTACGTGATGAACTACGAAATCAGCTCCAG | |
| | AGTAGCCTACGACACCGCTCATTACTGCCTG | |
| | CTTGACACGCTCGGCTGCGGTCTGGAAGCT | |
| | CTCGAATATCCGGCCTGTAAAAAACTGCTG | |
| | GGGCCAATTGTCCCCGGCACCGTCGTACCC | |
| | AACGGCGTGCGCGTTCCCGGAACTCAGTTT | |
| | CAGCTCGACCCCGTCCAGGCGGCATTTAAC | |
| | ATTGGCGCGATGATCCGTTGGCTCGATTTCA | |
| | ACGATACCTGGCTGGCGGCGGAGTGGGGGC | |
| | ATCCTTCCGACAACCTCGGCGGCATTCTGG | |
| | CAACGGCGGACTGGCTTTCGCGCAACGCGA | |
| | TCGCCAGCGGCAAAGCGCCGTTGACCATGA | |
| | AACAGGTGCTGACCGGAATGATCAAAGCCC | |
| | ATGAAATTCAGGGCTGCATCGCGCTGGAAA | |
| | ACTCCTTTAACCGCGTTGGTCTCGACCACGT | |
| | TCTGTTAGTGAAAGTGGCTTCCACCGCCGT | |
| | GGTCGCCGAAATGCTCGGCCTGACCCGCGA | |
| | GGAAATTCTCAACGCCGTTTCGCTGGCATG | |
| | GGTAGACGGACAGTCGCTGCGCACTTATCG | |
| | TCATGCACCGAACACCGGTACGCGTAAATC | |
| | CTGGGCGGCGGGCGATGCTACATCCCGCGC | |
| | GGTACGTCTGGCGCTGATGGCGAAAACGGG | |
| | CGAAATGGGTTACCCGTCAGCCCTGACCGC | |
| | GCCGGTGTGGGGTTTCTACGACGTCTCCTTT | |
| | AAAGGTGAGTCATTCCGCTTCCAGCGTCCG | |
| | TACGGTTCCTACGTCATGGAAAATGTGCTGT | |
| | TCAAAATCTCCTTCCCGGCGGAGTTCCACTC | |
| | CCAGACGGCAGTTGAAGCGGCGATGACGCT | |
| | CTATGAACAGATGCAGGCAGCAGGCAAAAC | |
| | GGCGGCAGATATCGAAAAAGTGACCATTCG | |
| | CACCCACGAAGCCTGTATTCGCATCATCGAC | |
| | AAAAAAGGGCCGCTCAATAACCCGGCAGAC | |
| | CGCGACCACTGCATTCAGTACATGGTGGCG | |
| | ATCCCGCTGCTGTTCGGACGCTTAACGGCG | |
| | GCAGATTACGAGGACAACGTTGCGCAAGAT | |
| | AAACGCATCGACGCCCTGCGCGAGAAGATC | |
| | AATTGCTTTGAAGATCCGGCGTTTACCGCTG | |
| | ACTACCACGACCCGGAAAAACGCGCCATCG | |
| | CCAATGCCATAACCCTTGAGTTCACCGACG | |
| | GCACACGATTTGAAGAAGTGGTGGTGGAGT | |
| | ACCCAATTGGTCATGCTCGCCGCCGTCAGG | |
| | ATGGCATTCCGAAGCTGGTCGATAAATTCAA | |
| | AATCAATCTCGCGCGCCAGTTCCCGACTCG | |
| | CCAGCAGCAGCGCATTCTGGAGGTTTCTCT | |
| | CGACAGAACTCGCCTGGAACAGATGCCGGT | |
| | CAATGAGTATCTCGACCTGTACGTCATTTAA | |
| | GTAAACGGCGGTAAGGCGTAAGTTCAACAGGA | |
| | GAGCATTATGTCTTTTAGCGAATTTTATCAG | |
| | CGTTCGATTAACGAACCGGAGAAGTTCTGG | |
| | GCCGAGCAGGCCCGGCGTATTGACTGGCAG | |
| | ACGCCCTTTACGCAAACGCTCGACCACAGC | |
| | AACCCGCCGTTTGCCCGTTGGTTTTGTGAAG | |
| | GCCGAACCAACTTGTGTCACAACGCTATCG | |
| | ACCGCTGGCTGGAGAAACAGCCAGAGGCGC | |
| | TGGCATTGATTGCCGTCTCTTCGGAAACAGA | |
| | GGAAGAGCGTACCTTTACCTTCCGCCAGTTA | |
| | CATGACGAAGTGAATGCGGTGGCGTCAATG | |
| | CTGCGCTCACTGGGCGTGCAGCGTGGCGAT | |
| | CGGGTGCTGGTGTATATGCCGATGATTGCC | |
| | GAAGCGCATATTACCCTGCTGGCCTGCGCG | |
| | CGCATTGGTGCTATTCACTCGGTGGTGTTTG | |
| | GGGGATTTGCTTCGCACAGCGTGGCAACGC | |
| | GAATTGATGACGCTAAACCGGTGCTGATTG | |
| | TCTCGGCTGATGCCGGGGCGCGCGGCGGTA | |
| | AAATCATTCCGTATAAAAAATTGCTCGACGA | |
| | TGCGATAAGTCAGGCACAGCATCAGCCGCG | |
| | TCACGTTTTACTGGTGGATCGCGGGCTGGC | |
| | GAAAATGGCGCGCGTTAGCGGGCGGGATGT | |
| | CGATTTCGCGTCGTTGCGCCATCAACACATC | |
| | GGCGCGCGGGTGCCGGTGGCATGGCTGGAA | |
| | TCCAACGAAACCTCCTGCATTCTCTACACCT | |
| | CCGGCACGACCGGCAAACCTAAAGGTGTGC | |
| | AGCGTGATGTCGGCGGATATGCGGTGGCGC | |
| | TGGCGACCTCGATGGACACCATTTTTGGCG | |
| | GCAAAGCGGGCGGCGTGTTCTTTTGTGCTT | |
| | CGGATATCGGCTGGGTGGTAGGGCATTCGT | |
| | ATATCGTTTACGCGCCGCTGCTGGCGGGGA | |
| | TGGCGACTATCGTTTACGAAGGATTGCCGA | |
| | CCTGGCCGGACTGCGGCGTGTGGTGGAAAA | |
| | TTGTCGAGAAATATCAGGTTAGCCGCATGTT | |
| | CTCAGCGCCGACCGCCATTCGCGTGCTGAA | |
| | AAAATTCCCTACCGCTGAAATTCGCAAACAC | |
| | GATCTTTCGTCGCTGGAAGTGCTCTATCTGG | |
| | CTGGAGAACCGCTGGACGAGCCGACCGCCA | |
| | GTTGGGTGAGCAATACGCTGGATGTGCCGG | |
| | TCATCGACAACTACTGGCAGACCGAATCCG | |
| | GCTGGCCGATTATGGCGATTGCTCGCGGTC | |
| | TGGATGACAGACCGACGCGTCTGGGAAGCC | |
| | CCGGCGTGCCGATGTATGGCTATAACGTGC | |
| | AGTTGCTCAATGAAGTCACCGGCGAACCGT | |
| | GTGGCGTCAATGAGAAAGGGATGCTGGTAG | |
| | TGGAGGGGCCATTGCCGCCAGGCTGTATTC | |
| | AAACCATCTGGGGCGACGACGACCGCTTTG | |
| | TGAAGACGTACTGGTCGCTGTTTTCCCGTCC | |
| | GGTGTACGCCACTTTTGACTGGGGCATCCG | |
| | CGATGCTGACGGTTATCACTTTATTCTCGGG | |
| | CGCACTGACGATGTGATTAACGTTGCCGGA | |
| | CATCGGCTGGGTACGCGTGAGATTGAAGAG | |
| | AGTATCTCCAGTCATCCGGGCGTTGCCGAA | |
| | GTGGCGGTGGTTGGGGTGAAAGATGCGCTG | |
| | AAAGGGCAGGTGGCGGTGGCGTTTGTCATT | |
| | CCGAAAGAGAGCGACAGTCTGGAAGACCGT | |
| | GAGGTGGCGCACTCGCAAGAGAAGGCGATT | |
| | ATGGCGCTGGTGGACAGCCAGATTGGCAAC | |
| | TTTGGCCGCCCGGCGCACGTCTGGTTTGTC | |
| | TCGCAATTGCCAAAAACGCGATCCGGAAAA | |
| | ATGCTGCGCCGCACGATCCAGGCGATTTGC | |
| | GAAGGACGCGATCCTGGGGATCTGACGACC | |
| | ATTGATGATCCGGCGTCGTTGGATCAGATC | |
| | CGCCAGGCGATGGAAGAGTAG | |
| Construct comprising | ATGTCTCTACACTCTCCAGGTAAAGCGTTTC | SEQ ID |
| a prpBCDE gene | GCGCTGCACTTAGCAAAGAAACCCCGTTGC | NO: 55 |
| cassette; (as shown in | AAATTGTTGGCACCATCAACGCTAACCATGC | |
| FIG. 20) ribosome | GCTGCTGGCGCAGCGTGCCGGATATCAGGC | |
| binding sites are | GATTTATCTCTCCGGCGGTGGCGTGGCGGC | |
| underlined; coding | AGGATCGCTGGGGCTGCCCGATCTCGGTAT | |
| region in bold | TTCTACTCTTGATGACGTGCTGACAGATATT | |
| | CGCCGTATCACCGACGTTTGTTCGCTGCCG | |
| | CTGCTGGTGGATGCGGATATCGGTTTTGGT | |
| | TCTTCAGCCTTTAACGTGGCGCGTACGGTG | |
| | AAATCAATGATTAAAGCCGGTGCGGCAGGA | |
| | TTGCATATTGAAGATCAGGTTGGTGCGAAA | |
| | CGCTGCGGTCATCGTCCGAATAAAGCGATC | |
| | GTCTCGAAAGAAGAGATGGTGGATCGGATC | |
| | CGCGCGGCGGTGGATGCGAAAACCGATCCT | |
| | GATTTTGTGATCATGGCGCGCACCGATGCG | |
| | CTGGCGGTAGAGGGGCTGGATGCGGCGATC | |
| | GAGCGTGCGCAGGCCTATGTTGAAGCGGGT | |
| | GCCGAAATGCTGTTCCCGGAGGCGATTACC | |
| | GAACTCGCCATGTATCGCCAGTTTGCCGAT | |
| | GCGGTGCAGGTGCCGATCCTCTCCAACATT | |
| | ACCGAATTTGGCGCAACACCGCTGTTTACCA | |
| | CCGACGAATTACGCAGCGCCCATGTCGCAA | |
| | TGGCGCTCTACCCGCTTTCAGCGTTTCGCGC | |
| | CATGAACCGCGCCGCTGAACATGTCTATAA | |
| | CATCCTGCGTCAGGAAGGCACACAGAAAAG | |
| | CGTCATCGACACCATGCAGACCCGCAACGA | |
| | GCTGTACGAAAGCATCAACTACTACCAGTAC | |
| | GAAGAGAAGCTCGACGACCTGTTTGCCCGT | |
| | GGTCAGGTGAAATAA | |
| | AAACGCCCGTTGGTTGTATTCGACAACCGATG | |
| | CCTGATGCGCCGCTGACGCGACTTATCAGGCC | |
| | TACGAGGTGAACTGAACTGTAGGTCGGATAAG | |
| | ACGCATAGCGTCGCATCCGACAACAATCTCGA | |
| | CCCTACAAATGATAACAATGACGAGGACAATA | |
| | TGAGCGACACAACGATCCTGCAAAACAGTA | |
| | CCCATGTCATTAAACCGAAAAAATCGGTGG | |
| | CACTTTCCGGCGTTCCGGCGGGCAATACGG | |
| | CGCTCTGCACCGTGGGTAAAAGCGGCAACG | |
| | ACCTGCATTACCGTGGCTACGATATTCTTGA | |
| | TCTGGCGGAACATTGTGAATTTGAAGAAGT | |
| | GGCGCACCTGCTGATCCACGGCAAACTGCC | |
| | AACCCGTGACGAACTCGCCGCCTACAAAAC | |
| | GAAACTGAAAGCCCTGCGTGGTTTACCGGC | |
| | TAACGTGCGTACCGTGCTGGAAGCCTTACC | |
| | GGCGGCGTCACACCCGATGGATGTTATGCG | |
| | CACCGGCGTTTCCGCGCTCGGCTGCACGCT | |
| | GCCAGAAAAAGAGGGGCACACCGTTTCTGG | |
| | TGCGCGGGATATTGCCGACAAACTGCTGGC | |
| | GTCACTTAGTTCGATTCTTCTCTACTGGTAT | |
| | CACTACAGCCACAACGGCGAACGCATCCAG | |
| | CCGGAAACTGATGACGACTCTATCGGCGGT | |
| | CACTTCCTGCATCTGCTGCACGGCGAAAAG | |
| | CCGTCGCAAAGCTGGGAAAAGGCGATGCAT | |
| | ATCTCGCTGGTGCTGTACGCCGAACACGAG | |
| | TTTAACGCTTCCACCTTTACCAGCCGGGTGA | |
| | TTGCGGGCACTGGCTCTGATATGTATTCCGC | |
| | CATTATTGGCGCGATTGGCGCACTGCGCGG | |
| | GCCGAAACACGGCGGGGCGAATGAAGTGTC | |
| | GCTGGAGATCCAGCAACGCTACGAAACGCC | |
| | GGGCGAAGCCGAAGCCGATATCCGCAAGCG | |
| | GGTGGAAAACAAAGAAGTGGTCATTGGTTT | |
| | TGGGCATCCGGTTTATACCATCGCCGACCC | |
| | GCGTCATCAGGTGATCAAACGTGTGGCGAA | |
| | GCAGCTCTCGCAGGAAGGCGGCTCGCTGAA | |
| | GATGTACAACATCGCCGATCGCCTGGAAAC | |
| | GGTGATGTGGGAGAGCAAAAAGATGTTCCC | |
| | CAATCTCGACTGGTTCTCCGCTGTTTCCTAC | |
| | AACATGATGGGTGTTCCCACCGAGATGTTC | |
| | ACACCACTGTTTGTTATCGCCCGCGTCACTG | |
| | GCTGGGCGGCGCACATTATCGAACAACGTC | |
| | AGGACAACAAAATTATCCGTCCTTCCGCCAA | |
| | TTATGTTGGACCGGAAGACCGCCAGTTTGT | |
| | CGCGCTGGATAAGCGCCAGTAA | |
| | ACCTCTACGAATAACAATAAGGAAACGTACCC | |
| | AATGTCAGCTCAAATCAACAACATCCGCCCG | |
| | GAATTTGATCGTGAAATCGTTGATATCGTCG | |
| | ATTACGTGATGAACTACGAAATCAGCTCCAG | |
| | AGTAGCCTACGACACCGCTCATTACTGCCTG | |
| | CTTGACACGCTCGGCTGCGGTCTGGAAGCT | |
| | CTCGAATATCCGGCCTGTAAAAAACTGCTG | |
| | GGGCCAATTGTCCCCGGCACCGTCGTACCC | |
| | AACGGCGTGCGCGTTCCCGGAACTCAGTTT | |
| | CAGCTCGACCCCGTCCAGGCGGCATTTAAC | |
| | ATTGGCGCGATGATCCGTTGGCTCGATTTCA | |
| | ACGATACCTGGCTGGCGGCGGAGTGGGGGC | |
| | ATCCTTCCGACAACCTCGGCGGCATTCTGG | |
| | CAACGGCGGACTGGCTTTCGCGCAACGCGA | |
| | TCGCCAGCGGCAAAGCGCCGTTGACCATGA | |
| | AACAGGTGCTGACCGGAATGATCAAAGCCC | |
| | ATGAAATTCAGGGCTGCATCGCGCTGGAAA | |
| | ACTCCTTTAACCGCGTTGGTCTCGACCACGT | |
| | TCTGTTAGTGAAAGTGGCTTCCACCGCCGT | |
| | GGTCGCCGAAATGCTCGGCCTGACCCGCGA | |
| | GGAAATTCTCAACGCCGTTTCGCTGGCATG | |
| | GGTAGACGGACAGTCGCTGCGCACTTATCG | |
| | TCATGCACCGAACACCGGTACGCGTAAATC | |
| | CTGGGCGGCGGGCGATGCTACATCCCGCGC | |
| | GGTACGTCTGGCGCTGATGGCGAAAACGGG | |
| | CGAAATGGGTTACCCGTCAGCCCTGACCGC | |
| | GCCGGTGTGGGGTTTCTACGACGTCTCCTTT | |
| | AAAGGTGAGTCATTCCGCTTCCAGCGTCCG | |
| | TACGGTTCCTACGTCATGGAAAATGTGCTGT | |
| | TCAAAATCTCCTTCCCGGCGGAGTTCCACTC | |
| | CCAGACGGCAGTTGAAGCGGCGATGACGCT | |
| | CTATGAACAGATGCAGGCAGCAGGCAAAAC | |
| | GGCGGCAGATATCGAAAAAGTGACCATTCG | |
| | CACCCACGAAGCCTGTATTCGCATCATCGAC | |
| | AAAAAAGGGCCGCTCAATAACCCGGCAGAC | |
| | CGCGACCACTGCATTCAGTACATGGTGGCG | |
| | ATCCCGCTGCTGTTCGGACGCTTAACGGCG | |
| | GCAGATTACGAGGACAACGTTGCGCAAGAT | |
| | AAACGCATCGACGCCCTGCGCGAGAAGATC | |
| | AATTGCTTTGAAGATCCGGCGTTTACCGCTG | |
| | ACTACCACGACCCGGAAAAACGCGCCATCG | |
| | CCAATGCCATAACCCTTGAGTTCACCGACG | |
| | GCACACGATTTGAAGAAGTGGTGGTGGAGT | |
| | ACCCAATTGGTCATGCTCGCCGCCGTCAGG | |
| | ATGGCATTCCGAAGCTGGTCGATAAATTCAA | |
| | AATCAATCTCGCGCGCCAGTTCCCGACTCG | |
| | CCAGCAGCAGCGCATTCTGGAGGTTTCTCT | |
| | CGACAGAACTCGCCTGGAACAGATGCCGGT | |
| | CAATGAGTATCTCGACCTGTACGTCATTTAA | |
| | GTAAACGGCGGTAAGGCGTAAGTTCAACAGGA | |
| | GAGCATTATGTCTTTTAGCGAATTTTATCAG | |
| | CGTTCGATTAACGAACCGGAGAAGTTCTGG | |
| | GCCGAGCAGGCCCGGCGTATTGACTGGCAG | |
| | ACGCCCTTTACGCAAACGCTCGACCACAGC | |
| | AACCCGCCGTTTGCCCGTTGGTTTTGTGAAG | |
| | GCCGAACCAACTTGTGTCACAACGCTATCG | |
| | ACCGCTGGCTGGAGAAACAGCCAGAGGCGC | |
| | TGGCATTGATTGCCGTCTCTTCGGAAACAGA | |
| | GGAAGAGCGTACCTTTACCTTCCGCCAGTTA | |
| | CATGACGAAGTGAATGCGGTGGCGTCAATG | |
| | CTGCGCTCACTGGGCGTGCAGCGTGGCGAT | |
| | CGGGTGCTGGTGTATATGCCGATGATTGCC | |
| | GAAGCGCATATTACCCTGCTGGCCTGCGCG | |
| | CGCATTGGTGCTATTCACTCGGTGGTGTTTG | |
| | GGGGATTTGCTTCGCACAGCGTGGCAACGC | |
| | GAATTGATGACGCTAAACCGGTGCTGATTG | |
| | TCTCGGCTGATGCCGGGGCGCGCGGCGGTA | |
| | AAATCATTCCGTATAAAAAATTGCTCGACGA | |
| | TGCGATAAGTCAGGCACAGCATCAGCCGCG | |
| | TCACGTTTTACTGGTGGATCGCGGGCTGGC | |
| | GAAAATGGCGCGCGTTAGCGGGCGGGATGT | |
| | CGATTTCGCGTCGTTGCGCCATCAACACATC | |
| | GGCGCGCGGGTGCCGGTGGCATGGCTGGAA | |
| | TCCAACGAAACCTCCTGCATTCTCTACACCT | |
| | CCGGCACGACCGGCAAACCTAAAGGTGTGC | |
| | AGCGTGATGTCGGCGGATATGCGGTGGCGC | |
| | TGGCGACCTCGATGGACACCATTTTTGGCG | |
| | GCAAAGCGGGCGGCGTGTTCTTTTGTGCTT | |
| | CGGATATCGGCTGGGTGGTAGGGCATTCGT | |
| | ATATCGTTTACGCGCCGCTGCTGGCGGGGA | |
| | TGGCGACTATCGTTTACGAAGGATTGCCGA | |
| | CCTGGCCGGACTGCGGCGTGTGGTGGAAAA | |
| | TTGTCGAGAAATATCAGGTTAGCCGCATGTT | |
| | CTCAGCGCCGACCGCCATTCGCGTGCTGAA | |
| | AAAATTCCCTACCGCTGAAATTCGCAAACAC | |
| | GATCTTTCGTCGCTGGAAGTGCTCTATCTGG | |
| | CTGGAGAACCGCTGGACGAGCCGACCGCCA | |
| | GTTGGGTGAGCAATACGCTGGATGTGCCGG | |
| | TCATCGACAACTACTGGCAGACCGAATCCG | |
| | GCTGGCCGATTATGGCGATTGCTCGCGGTC | |
| | TGGATGACAGACCGACGCGTCTGGGAAGCC | |
| | CCGGCGTGCCGATGTATGGCTATAACGTGC | |
| | AGTTGCTCAATGAAGTCACCGGCGAACCGT | |
| | GTGGCGTCAATGAGAAAGGGATGCTGGTAG | |
| | TGGAGGGGCCATTGCCGCCAGGCTGTATTC | |
| | AAACCATCTGGGGCGACGACGACCGCTTTG | |
| | TGAAGACGTACTGGTCGCTGTTTTCCCGTCC | |
| | GGTGTACGCCACTTTTGACTGGGGCATCCG | |
| | CGATGCTGACGGTTATCACTTTATTCTCGGG | |
| | CGCACTGACGATGTGATTAACGTTGCCGGA | |
| | CATCGGCTGGGTACGCGTGAGATTGAAGAG | |
| | AGTATCTCCAGTCATCCGGGCGTTGCCGAA | |
| | GTGGCGGTGGTTGGGGTGAAAGATGCGCTG | |
| | AAAGGGCAGGTGGCGGTGGCGTTTGTCATT | |
| | CCGAAAGAGAGCGACAGTCTGGAAGACCGT | |
| | GAGGTGGCGCACTCGCAAGAGAAGGCGATT | |
| | ATGGCGCTGGTGGACAGCCAGATTGGCAAC | |
| | TTTGGCCGCCCGGCGCACGTCTGGTTTGTC | |
| | TCGCAATTGCCAAAAACGCGATCCGGAAAA | |
| | ATGCTGCGCCGCACGATCCAGGCGATTTGC | |
| | GAAGGACGCGATCCTGGGGATCTGACGACC | |
| | ATTGATGATCCGGCGTCGTTGGATCAGATC | |
| | CGCCAGGCGATGGAAGAGTAG | |
| prpB sequence | ATGTCTCTACACTCTCCAGGTAAAGCGTTTCGC | SEQ ID |
| (comprised in the | GCTGCACTTAGCAAAGAAACCCCGTTGCAAAT | NO: 56 |
| prpBCDE construct | TGTTGGCACCATCAACGCTAACCATGCGCTGCT | |
| shown in FIG. 20) | GGCGCAGCGTGCCGGATATCAGGCGATTTATC | |
| | TCTCCGGCGGTGGCGTGGCGGCAGGATCGCTG | |
| | GGGCTGCCCGATCTCGGTATTTCTACTCTTGAT | |
| | GACGTGCTGACAGATATTCGCCGTATCACCGA | |
| | CGTTTGTTCGCTGCCGCTGCTGGTGGATGCGGA | |
| | TATCGGTTTTGGTTCTTCAGCCTTTAACGTGGC | |
| | GCGTACGGTGAAATCAATGATTAAAGCCGGTG | |
| | CGGCAGGATTGCATATTGAAGATCAGGTTGGT | |
| | GCGAAACGCTGCGGTCATCGTCCGAATAAAGC | |
| | GATCGTCTCGAAAGAAGAGATGGTGGATCGGA | |
| | TCCGCGCGGCGGTGGATGCGAAAACCGATCCT | |
| | GATTTTGTGATCATGGCGCGCACCGATGCGCT | |
| | GGCGGTAGAGGGGCTGGATGCGGCGATCGAGC | |
| | GTGCGCAGGCCTATGTTGAAGCGGGTGCCGAA | |
| | ATGCTGTTCCCGGAGGCGATTACCGAACTCGC | |
| | CATGTATCGCCAGTTTGCCGATGCGGTGCAGG | |
| | TGCCGATCCTCTCCAACATTACCGAATTTGGCG | |
| | CAACACCGCTGTTTACCACCGACGAATTACGC | |
| | AGCGCCCATGTCGCAATGGCGCTCTACCCGCTT | |
| | TCAGCGTTTCGCGCCATGAACCGCGCCGCTGA | |
| | ACATGTCTATAACATCCTGCGTCAGGAAGGCA | |
| | CACAGAAAAGCGTCATCGACACCATGCAGACC | |
| | CGCAACGAGCTGTACGAAAGCATCAACTACTA | |
| | CCAGTACGAAGAGAAGCTCGACGACCTGTTTG | |
| | CCCGTGGTCAGGTGAAATAA | |
| prpC sequence | ATGAGCGACACAACGATCCTGCAAAACAGTAC | SEQ ID |
| (comprised in the | CCATGTCATTAAACCGAAAAAATCGGTGGCAC | NO: 57 |
| prpBCDE construct | TTTCCGGCGTTCCGGCGGGCAATACGGCGCTCT | |
| shown in FIG. 20) | GCACCGTGGGTAAAAGCGGCAACGACCTGCAT | |
| | TACCGTGGCTACGATATTCTTGATCTGGCGGAA | |
| | CATTGTGAATTTGAAGAAGTGGCGCACCTGCT | |
| | GATCCACGGCAAACTGCCAACCCGTGACGAAC | |
| | TCGCCGCCTACAAAACGAAACTGAAAGCCCTG | |
| | CGTGGTTTACCGGCTAACGTGCGTACCGTGCTG | |
| | GAAGCCTTACCGGCGGCGTCACACCCGATGGA | |
| | TGTTATGCGCACCGGCGTTTCCGCGCTCGGCTG | |
| | CACGCTGCCAGAAAAAGAGGGGCACACCGTTT | |
| | CTGGTGCGCGGGATATTGCCGACAAACTGCTG | |
| | GCGTCACTTAGTTCGATTCTTCTCTACTGGTAT | |
| | CACTACAGCCACAACGGCGAACGCATCCAGCC | |
| | GGAAACTGATGACGACTCTATCGGCGGTCACT | |
| | TCCTGCATCTGCTGCACGGCGAAAAGCCGTCG | |
| | CAAAGCTGGGAAAAGGCGATGCATATCTCGCT | |
| | GGTGCTGTACGCCGAACACGAGTTTAACGCTT | |
| | CCACCTTTACCAGCCGGGTGATTGCGGGCACT | |
| | GGCTCTGATATGTATTCCGCCATTATTGGCGCG | |
| | ATTGGCGCACTGCGCGGGCCGAAACACGGCGG | |
| | GGCGAATGAAGTGTCGCTGGAGATCCAGCAAC | |
| | GCTACGAAACGCCGGGCGAAGCCGAAGCCGAT | |
| | ATCCGCAAGCGGGTGGAAAACAAAGAAGTGG | |
| | TCATTGGTTTTGGGCATCCGGTTTATACCATCG | |
| | CCGACCCGCGTCATCAGGTGATCAAACGTGTG | |
| | GCGAAGCAGCTCTCGCAGGAAGGCGGCTCGCT | |
| | GAAGATGTACAACATCGCCGATCGCCTGGAAA | |
| | CGGTGATGTGGGAGAGCAAAAAGATGTTCCCC | |
| | AATCTCGACTGGTTCTCCGCTGTTTCCTACAAC | |
| | ATGATGGGTGTTCCCACCGAGATGTTCACACC | |
| | ACTGTTTGTTATCGCCCGCGTCACTGGCTGGGC | |
| | GGCGCACATTATCGAACAACGTCAGGACAACA | |
| | AAATTATCCGTCCTTCCGCCAATTATGTTGGAC | |
| | CGGAAGACCGCCAGTTTGTCGCGCTGGATAAG | |
| | CGCCAGTAA | |
| prpD sequence | ATGTCAGCTCAAATCAACAACATCCGCCCGGA | SEQ ID |
| (comprised in the | ATTTGATCGTGAAATCGTTGATATCGTCGATTA | NO: 58 |
| prpBCDE construct | CGTGATGAACTACGAAATCAGCTCCAGAGTAG | |
| shown in FIG. 20) | CCTACGACACCGCTCATTACTGCCTGCTTGACA | |
| | CGCTCGGCTGCGGTCTGGAAGCTCTCGAATAT | |
| | CCGGCCTGTAAAAAACTGCTGGGGCCAATTGT | |
| | CCCCGGCACCGTCGTACCCAACGGCGTGCGCG | |
| | TTCCCGGAACTCAGTTTCAGCTCGACCCCGTCC | |
| | AGGCGGCATTTAACATTGGCGCGATGATCCGT | |
| | TGGCTCGATTTCAACGATACCTGGCTGGCGGC | |
| | GGAGTGGGGGCATCCTTCCGACAACCTCGGCG | |
| | GCATTCTGGCAACGGCGGACTGGCTTTCGCGC | |
| | AACGCGATCGCCAGCGGCAAAGCGCCGTTGAC | |
| | CATGAAACAGGTGCTGACCGGAATGATCAAAG | |
| | CCCATGAAATTCAGGGCTGCATCGCGCTGGAA | |
| | AACTCCTTTAACCGCGTTGGTCTCGACCACGTT | |
| | CTGTTAGTGAAAGTGGCTTCCACCGCCGTGGTC | |
| | GCCGAAATGCTCGGCCTGACCCGCGAGGAAAT | |
| | TCTCAACGCCGTTTCGCTGGCATGGGTAGACG | |
| | GACAGTCGCTGCGCACTTATCGTCATGCACCG | |
| | AACACCGGTACGCGTAAATCCTGGGCGGCGGG | |
| | CGATGCTACATCCCGCGCGGTACGTCTGGCGC | |
| | TGATGGCGAAAACGGGCGAAATGGGTTACCCG | |
| | TCAGCCCTGACCGCGCCGGTGTGGGGTTTCTAC | |
| | GACGTCTCCTTTAAAGGTGAGTCATTCCGCTTC | |
| | CAGCGTCCGTACGGTTCCTACGTCATGGAAAA | |
| | TGTGCTGTTCAAAATCTCCTTCCCGGCGGAGTT | |
| | CCACTCCCAGACGGCAGTTGAAGCGGCGATGA | |
| | CGCTCTATGAACAGATGCAGGCAGCAGGCAAA | |
| | ACGGCGGCAGATATCGAAAAAGTGACCATTCG | |
| | CACCCACGAAGCCTGTATTCGCATCATCGACA | |
| | AAAAAGGGCCGCTCAATAACCCGGCAGACCGC | |
| | GACCACTGCATTCAGTACATGGTGGCGATCCC | |
| | GCTGCTGTTCGGACGCTTAACGGCGGCAGATT | |
| | ACGAGGACAACGTTGCGCAAGATAAACGCATC | |
| | GACGCCCTGCGCGAGAAGATCAATTGCTTTGA | |
| | AGATCCGGCGTTTACCGCTGACTACCACGACC | |
| | CGGAAAAACGCGCCATCGCCAATGCCATAACC | |
| | CTTGAGTTCACCGACGGCACACGATTTGAAGA | |
| | AGTGGTGGTGGAGTACCCAATTGGTCATGCTC | |
| | GCCGCCGTCAGGATGGCATTCCGAAGCTGGTC | |
| | GATAAATTCAAAATCAATCTCGCGCGCCAGTT | |
| | CCCGACTCGCCAGCAGCAGCGCATTCTGGAGG | |
| | TTTCTCTCGACAGAACTCGCCTGGAACAGATG | |
| | CCGGTCAATGAGTATCTCGACCTGTACGTCATT | |
| | TAA | |
| prpE sequence | ATGTCTTTTAGCGAATTTTATCAGCGTTCGATT | SEQ ID |
| (comprised in the | AACGAACCGGAGAAGTTCTGGGCCGAGCAGGC | NO: 25 |
| prpBCDE construct | CCGGCGTATTGACTGGCAGACGCCCTTTACGC | |
| shown in FIG. 20) | AAACGCTCGACCACAGCAACCCGCCGTTTGCC | |
| | CGTTGGTTTTGTGAAGGCCGAACCAACTTGTGT | |
| | CACAACGCTATCGACCGCTGGCTGGAGAAACA | |
| | GCCAGAGGCGCTGGCATTGATTGCCGTCTCTTC | |
| | GGAAACAGAGGAAGAGCGTACCTTTACCTTCC | |
| | GCCAGTTACATGACGAAGTGAATGCGGTGGCG | |
| | TCAATGCTGCGCTCACTGGGCGTGCAGCGTGG | |
| | CGATCGGGTGCTGGTGTATATGCCGATGATTG | |
| | CCGAAGCGCATATTACCCTGCTGGCCTGCGCG | |
| | CGCATTGGTGCTATTCACTCGGTGGTGTTTGGG | |
| | GGATTTGCTTCGCACAGCGTGGCAACGCGAAT | |
| | TGATGACGCTAAACCGGTGCTGATTGTCTCGG | |
| | CTGATGCCGGGGCGCGCGGCGGTAAAATCATT | |
| | CCGTATAAAAAATTGCTCGACGATGCGATAAG | |
| | TCAGGCACAGCATCAGCCGCGTCACGTTTTACT | |
| | GGTGGATCGCGGGCTGGCGAAAATGGCGCGCG | |
| | TTAGCGGGCGGGATGTCGATTTCGCGTCGTTGC | |
| | GCCATCAACACATCGGCGCGCGGGTGCCGGTG | |
| | GCATGGCTGGAATCCAACGAAACCTCCTGCAT | |
| | TCTCTACACCTCCGGCACGACCGGCAAACCTA | |
| | AAGGTGTGCAGCGTGATGTCGGCGGATATGCG | |
| | GTGGCGCTGGCGACCTCGATGGACACCATTTTT | |
| | GGCGGCAAAGCGGGCGGCGTGTTCTTTTGTGC | |
| | TTCGGATATCGGCTGGGTGGTAGGGCATTCGT | |
| | ATATCGTTTACGCGCCGCTGCTGGCGGGGATG | |
| | GCGACTATCGTTTACGAAGGATTGCCGACCTG | |
| | GCCGGACTGCGGCGTGTGGTGGAAAATTGTCG | |
| | AGAAATATCAGGTTAGCCGCATGTTCTCAGCG | |
| | CCGACCGCCATTCGCGTGCTGAAAAAATTCCC | |
| | TACCGCTGAAATTCGCAAACACGATCTTTCGTC | |
| | GCTGGAAGTGCTCTATCTGGCTGGAGAACCGC | |
| | TGGACGAGCCGACCGCCAGTTGGGTGAGCAAT | |
| | ACGCTGGATGTGCCGGTCATCGACAACTACTG | |
| | GCAGACCGAATCCGGCTGGCCGATTATGGCGA | |
| | TTGCTCGCGGTCTGGATGACAGACCGACGCGT | |
| | CTGGGAAGCCCCGGCGTGCCGATGTATGGCTA | |
| | TAACGTGCAGTTGCTCAATGAAGTCACCGGCG | |
| | AACCGTGTGGCGTCAATGAGAAAGGGATGCTG | |
| | GTAGTGGAGGGGCCATTGCCGCCAGGCTGTAT | |
| | TCAAACCATCTGGGGCGACGACGACCGCTTTG | |
| | TGAAGACGTACTGGTCGCTGTTTTCCCGTCCGG | |
| | TGTACGCCACTTTTGACTGGGGCATCCGCGATG | |
| | CTGACGGTTATCACTTTATTCTCGGGCGCACTG | |
| | ACGATGTGATTAACGTTGCCGGACATCGGCTG | |
| | GGTACGCGTGAGATTGAAGAGAGTATCTCCAG | |
| | TCATCCGGGCGTTGCCGAAGTGGCGGTGGTTG | |
| | GGGTGAAAGATGCGCTGAAAGGGCAGGTGGC | |
| | GGTGGCGTTTGTCATTCCGAAAGAGAGCGACA | |
| | GTCTGGAAGACCGTGAGGTGGCGCACTCGCAA | |
| | GAGAAGGCGATTATGGCGCTGGTGGACAGCCA | |
| | GATTGGCAACTTTGGCCGCCCGGCGCACGTCT | |
| | GGTTTGTCTCGCAATTGCCAAAAACGCGATCC | |
| | GGAAAAATGCTGCGCCGCACGATCCAGGCGAT | |
| | TTGCGAAGGACGCGATCCTGGGGATCTGACGA | |
| | CCATTGATGATCCGGCGTCGTTGGATCAGATCC | |
| | GCCAGGCGATGGAAGAGTAG | |

Next, the rate of propionate consumption of genetically engineered bacteria comprising the 2-Methylcitrate Cycle circuit is assessed in vitro.

Cultures of E. coli Nissle transformed with the plasmid comprising the prpBCDE circuit driven by the tet promoter and wild type control Nissle are grown overnight and then diluted 1:200 in LB. ATC is added to the cultures of the strain containing the prpE-phaBCA construct plasmid at a concentration of 100 ng/mL to induce expression of the prpBCDE genes and the cells are grown with shaking at 250 rpm After 2 hrs of incubation, cells are pelleted down, washed, and resuspended in 1 mL M9 medium supplemented with glucose (0.5%) and propionate (8 mM) at a concentration of ˜10⁹ cfu/ml bacteria. Aliquots are collected at 0 hrs, 2 hrs, and 4 hrs for propionate quantification and the catabolic rate is calculated.

### Example 15. Propionate Quantification in Bacterial Supernatant by LC-MS/MS

### Sample Preparation

Sodium propionate stock (10 mg/mL) in water was prepared, aliquoted in 1.5 mL microcentrifuge tubes (100 μL), and stored at −20° C. From the stock, Sodium propionate standards (1000, 500, 250, 100, 20, 4, 0.8 μg/mL) were prepared in water. Next, 25 μL of sample (bacterial supernatant and standards) was mixed with 75 μL of ACN/H₂O (45:30, v/v) containing 10 μg/mL of sodium propionate-d5 in a round-bottom 96-well plate. The plates were heat sealed with a PierceASeal foil and mixed well.

In a V-bottom 96-well polypropylene plate, 5 μL of diluted samples were added to 95 μL of derivatization mix (20 mM EDC [N-(3-Dimethylaminopropyl)-N′-ethylcarbodiimide hydrochloride] and 20 mM TFEA [2,2,2-Trifluoroethylamine hydrochloride] in 10 mM MES buffer pH 4.0). The plates were heat sealed with a ThermASeal foil and mixed well. The samples were incubated at RT for 1 hr for derivatization and then centrifuged at 4000 rpm for 5 min.

Next, 20 μL of the solution were transferred into a round-bottom 96-well plate, and 180 uL 0.1% formic acid in water was added to the samples. The plates were heat-sealed and mixed as described above.

### LC-MS/MS Method

Derivatized propionate was measured by liquid chromatography coupled to tandem mass spectrometry (LC-MS/MS) using a Thermo TSQ Quantum Max triple quadrupole mass spectrometer. HPLC Method details are described in Table 34 and Table 35. Tandem Mass Spectrometry details are described in Table 20.

TABLE 34
HPLC Method Details

| | |
|---|---|
| Column | Aquasil C18(2) column, 5 μm (50 × 2.1 mm) |
| Mobile Phase A | 100% H2O, 0.1% Formic Acid |
| Mobile Phase B | 100% ACN, 0.1% Formic Acid |
| Injection volume | 10 uL |

TABLE 35
HPLC Method Details

| Time (min) | Flow Rate | A % | B % |
|---|---|---|---|
| −0.5 | 250 | 100 | 0 |
| 0.5 | 250 | 100 | 0 |
| 2.5 | 250 | 10 | 90 |
| 3.5 | 250 | 10 | 90 |
| 3.51 | 250 | 0 | 10 |

TABLE 36
Tandem Mass Spectrometry Details

| | |
|---|---|
| Ion Source | HESI-II |
| Polarity | Positive |

SRM transitions

| | |
|---|---|
| Sodium propionate | 156.2/57.1 |
| Sodium propionate-d5 | 161.0/62.1 |

### Example 16. Acetylcarnitine and Propionylcarnitine Quantification in Plasma and Urine by LC-MS/MS

### Sample Preparation

Acetylcarnitine and Propionylcarnitine stock (10 mg/mL) was prepared in water, aliquoted into 1.5 mL microcentrifuge tubes (100 μL), and stored at −20° C. Standards of 250, 100, 20, 4, 0.8, 0.16, 0.032 μg/mL were prepared in water. Sample (10 μL) and standards were mixed with 90 μL of ACN/MeOH/H₂O (60:20:10, v/v) containing 1 μg/mL of Acetylcarnitine-d3 and Propionylcarnitine-d3 in the final solution) in a V-bottom 96-well plate. The plate was heat sealed with a AlumASeal foil, mixed well, and centrifuged at 4000 rpm for 5 min Next, 20 μL of the solution was transferred into a round-bottom 96-well plate, and 180 uL 0.1% formic acid in water was added to the sample. The plate was heat-sealed with a ClearASeal sheet and mixed well.

### LC-MS/MS Method

Propionylcarnitine and Acetylcarnitine were measured by liquid chromatography coupled to tandem mass spectrometry (LC-MS/MS) using a Thermo TSQ Quantum Max triple quadrupole mass spectrometer. HPLC Method details are described in Table 37 and Table 38. Tandem Mass Spectrometry details are described in Table 39.

TABLE 37
HPLC Method Details

| | |
|---|---|
| Column | HILIC column, 2.6 μm (100 × 2.1 mm) |
| Mobile Phase A | 100% H2O, 0.1% Formic Acid |
| Mobile Phase B | 100% ACN, 0.1% Formic Acid |
| Injection volume | 10 uL |

TABLE 38
HPLC Method Details

| Time (min) | Flow Rate (μL/min) | A % | B % |
|---|---|---|---|
| −0.5 | 250 | 100 | 0 |
| 0.5 | 250 | 100 | 0 |
| 2.5 | 250 | 10 | 90 |
| 3.5 | 250 | 10 | 90 |
| 3.51 | 250 | 0 | 10 |

TABLE 39
Tandem Mass Spectrometry Details

| | |
|---|---|
| Ion Source | HESI-II |
| Polarity | Positive |

SRM transitions

| | |
|---|---|
| Acetylcarnitine | 204.1/85.2 |
| Acetylcarnitine-d3 | 207.1/85.2 |
| Propionylcarnitine | 218.1/85.2 |
| Propionylcarnitine-d3 | 221.1/85.2 |

### Example 17. Propionate, 2-Methylcitrate, Propionylglycine, and Tigloylglycine Quantification in Plasma and Urine by LC-MS/MS

### Sample Preparation

Stocks of 10 mg/mL Sodium propionate, 2-Methylcitrate, Propionylglycine, and Tigloylglycine in water were prepared, aliquoted in 1.5 mL microcentrifuge tubes (100 μL), and stored at −20° C. Standards of 500, 250, 100, 20, 4, 0.8, 0.16, 0.032 μg/mL of each of the stocks were prepared in water. On ice, 10 μL of sample (and standards) were pipetted into a V-bottom polypropylene 96-well plate, and 90 μL of the derivatizing solution containing 50 mM of 2-Hydrazinoquinoline (2-HQ), dipyridyl disulfide, and triphenylphospine in acetonitrile with 5 ug/mL of Sodium propionate-13C3 and 2-Methylcitrate-d3 were added into the final solution. The plate was heat sealed with a ThermASeal foil and mixed well. The samples were incubated at 60° C. for 1 hr for derivatization and then centrifuged at 4000 rpm for 5 min Next, 20 μL of the derivatized samples were added to 180 μL of 0.1% formic acid in water/ACN (140:40) in a round-bottom 96-well plate. The plate was heat sealed with a ClearASeal sheet and mix well.

### LC-MS/MS Method

Derivatized metabolites were measured by liquid chromatography coupled to tandem mass spectrometry (LC-MS/MS) using a Thermo TSQ Quantum Max triple quadrupole mass spectrometer. HPLC Method details are described in Table 40 and Table 41. Tandem Mass Spectrometry details are described in Table 42.

TABLE 40
HPLC Method Details

| | |
|---|---|
| Column | C18 column, 5 μm (100 × 2 mm) |
| Mobile Phase A | 100% H2O, 0.1% Formic Acid |
| Mobile Phase B | 100% ACN, 0.1% Formic Acid |
| Injection volume | 10 uL |

TABLE 41
HPLC Method Details

| Time (min) | Flow Rate (μL/min) | A % | B % |
|---|---|---|---|
| 0 | 500 | 95 | 5 |
| 0.9 | 500 | 95 | 5 |
| 1.0 | 500 | 72.5 | 27.5 |
| 2.5 | 500 | 60 | 40 |
| 2.6 | 500 | 10 | 90 |
| 4.5 | 500 | 10 | 90 |
| 4.51 | 500 | 95 | 5 |
| 4.75 | 500 | 95 | 5 |

TABLE 42
Tandem Mass Spectrometry Details

| | |
|---|---|
| Ion Source | HESI-II |
| Polarity | Positive |

SRM transitions

| | |
|---|---|
| Sodium propionate | 216.1/160.1 |
| Sodium propionate-13C3 | 219.1/160.1 |
| 2-Methylcitrate | 489.2/471.2 |
| 2-Methylcitrate-d3 | 492.2/474.2 |
| Propionylglycine | 273.1/172.2* |
| Tigloylglycine | 299.1/160.1* |

*Quantified using external calibration (without internal standard)

### Example 18. In Vivo Studies Demonstrating that the Engineered Bacterial Cells Decrease Propionate Concentration

For in vivo studies, a hypomorphic mouse model of propionic acidemia is used (see, for example Guenzel et al., 2013). Alternatively, a PCCA−/− knock-out mouse or a mouse model of methylmalonic acidemia can be used (see, for example, Miyazaki et al., 2001 or Peters et al., 2012). Briefly, blood levels of methylcitrate, acetylcarnitine, and/or propionylcarnitine are measured in the mice prior to administration of the engineered bacteria on day 0. On day 1, cultures of E. coli Nissle containing pTet-prpBCDE and/or pTet-mctC are administered to three wild-type mice and three hypomorph mice once daily for a week. In addition, three hypomorph mice are administered PBS as a control once daily for a week. Treatment efficacy is determined, for example, by measuring blood levels of methylcitrate, acetylcarnitine, and/or propionylcarnitine. A decrease in blood levels of methylcitrate, acetylcarnitine, and/or propionylcarnitine after treatment with the engineered bacterial cells indicates that the engineered bacterial cells are effective for treating propionic acidemia and methylmalonic acidemia. Additionally, throughout the study, phenotypes of the mice can also be analyzed. A decrease in the number of symptoms associated with PA or MMA, for example, seizures, further indicates the efficacy of the engineered bacterial cells for treating PA and MMA.

### Example 19. Diet-Induced Changes in Plasma Biomarkers in PCCAA138T Hypomorph Mice Gavaged with PHA Pathway and MMCA Pathway Strains on Normal Chow

The efficacy of two strains, one expressing PHA pathway genes (PHA), and the other expressing MMCA (MMCA) pathway genes in vivo was assessed using a PCCAA138T hypomorph mouse model. Both strains used in the study were plasmid based strains expressing the pathway genes under the control of tetracycline and or arabinose inducible promoters. The PHA strain is described, e.g., in Example 9 and FIG. 10C and FIG. 11 and elsewhere herein. The MMCA strain is described, e.g., in Example 12 and FIG. 15C, FIG. 16A, and FIG. 16B and elsewhere herein.

On day −7, PCCAA138T hypomorph mice (females 14-18 weeks of age) were placed on normal chow and water. Mice were kept on regular chow throughout experiment.

On day 1, animals were randomized into treatment groups. Mice were bled and urine was collected (T=0) to obtain baseline plasma and urine biomarker levels. Mice were grouped as follows: Group 1: H₂O (n=10); Group 2: wild type Nissle with streptomycin resistance (n=10); Group 3: PHA strain (n=10); Group 4: MMCA strain (n=10). For Groups 2, 3 and 4 mice were gavaged with 10e10 CFU/dose in 100 unclose. Group 1 was dosed with 100 ul H2O. ATC (20 ng/mL) and 5% Sucrose was added to the drinking water.
On days 2 and 3, mice were dosed twice daily with 100 ul bacteria (10e10 CFU/dose) or H₂O (Group 1). On day 4, mice were dosed once with 100 ul bacteria (10e10 CFU/dose) or water and animals were weighed, blood was drawn and urine was collected at 4 hours post dose for LC/MS analysis.

To prepare the MMCA strain for this study, cultures comprising the two plasmid based MMCA pathway circuits, were grown overnight in LB and 50 ug/mL Ampicillin and then diluted 1:100 in LB. The cells were grown with shaking (250 rpm) to early log phase with the appropriate antibiotics (2 hours). Anhydrous tetracycline (ATC, 100 ng/ml) and arabinose (10 mM) was added to cultures to induce expression of the constructs, and bacteria were grown for another 3 hours. Prior to administration, cells were concentrated 200× and frozen (15% glycerol, 2 g/L glucose, in PBS). Cells were thawed on ice, and diluted in PBS to the appropriate concentration for dosing.

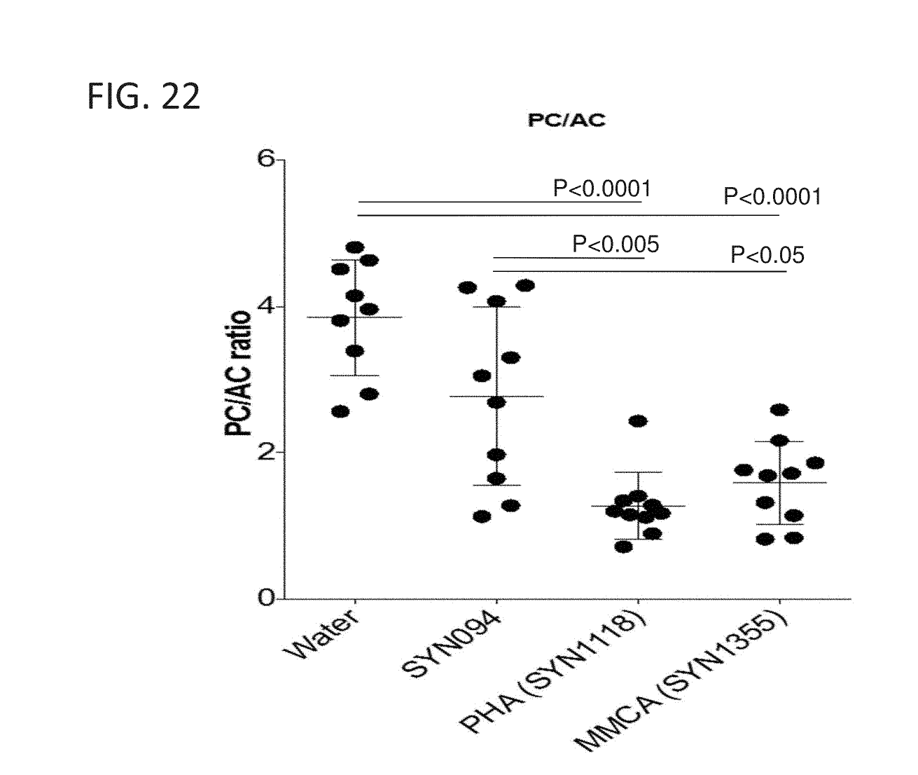

To prepare the PHA strain for this study, cultures of strains comprising the plasmid-based PHA pathway circuits, were grown overnight in LB and 50 ug/mL Ampicillin and then diluted 1:100 in LB. Cells were diluted 1:100 in LB, grown for 2 h aerobically, then ATC was added to cultures at a concentration of 100 ng/mL and cells were grown for an additional 3 hours. Prior to administration, cells were concentrated 200× and frozen (15% glycerol, 2 g/L glucose, in PBS). Cells were thawed on ice, and diluted in PBS to the appropriate concentration for dosing.
Results in FIG. 22 show that the ratio of propionyl carnitine to acetyl carnitine is reduced in both PHA and MMCA strains as compared to streptomycin resistant Nissle and the water controls.

### Example 20. Diet-Induced Changes in Plasma and Urinary Biomarkers in PCCAA138T Hypomorph Mice Gavaged with PHA Pathway and MMCA Pathway Strains on High Protein Diet

The efficacy of two strains, one expressing PHA pathway genes (PHA), and the other expressing MMCA pathway genes (MMCA) in vivo was assessed using a PCCAA138T hypomorph mouse model. Both strains used in the study were plasmid based strains expressing the pathway genes under the control of tetracycline inducible promoters. The PHA strain is described e.g., in Example 9 and FIG. 10C and FIG. 11 and elsewhere herein. The MMCA strain is described, e.g., in Example 12 and FIG. 15C, FIG. 16A, and FIG. 16B and elsewhere herein.

On day −7, animals (PCCAA138T hypomorph mice) were placed on normal chow and water. On day 1, animals were randomized into treatment groups. Mice were bled and urine was collected (T=0) to obtain baseline plasma and urine biomarker levels. Mice were grouped as follows: Group 1: H₂O (n=10); Group 2: wild type Nissle with streptomycin resistance (n=10); Group 3: PHA strain (n=10); Group 4: MMCA strain (n=10). Mice were placed on high protein chow. For Groups 2, 3 and 4 mice were gavaged with 10e10 CFU/dose in 100 Otiose. Group 1 was dosed with 100 ul H2O.

On days 2 through 5, mice were dosed twice daily with 100 ul bacteria (10e10 CFUs/dose) or H₂O (Group 1). On day 6, mice were dosed once with 100 ul bacteria (10e10 CFUs/dose) or water and animals were weighed, blood was drawn and urine was collected at 4 hours post dose for LC/MS analysis.

To prepare the MMCA strain for this study, cultures comprising the two plasmid based MMCA pathway circuits, were grown overnight in LB and 50 ug/mL ampicillin and then diluted 1:100 in LB. The cells were grown with shaking (250 rpm) to early log phase with the appropriate antibiotics (1.5 h). Anhydrous tetracycline (ATC, 100 ng/ml) and arabinose (10 mM) was added to cultures to induce expression of the constructs, and bacteria were grown for another 2.5 hours. Prior to administration, cells were concentrated 200× and frozen (15% glycerol, 2 g/L glucose, in PBS). Cells were thawed on ice, and diluted in PBS to the appropriate concentration for dosing.

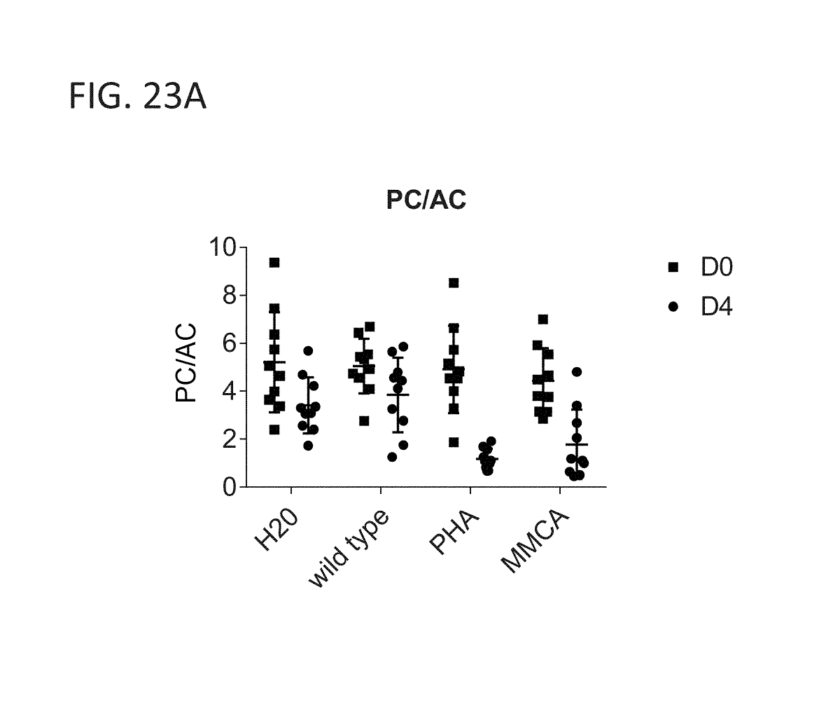

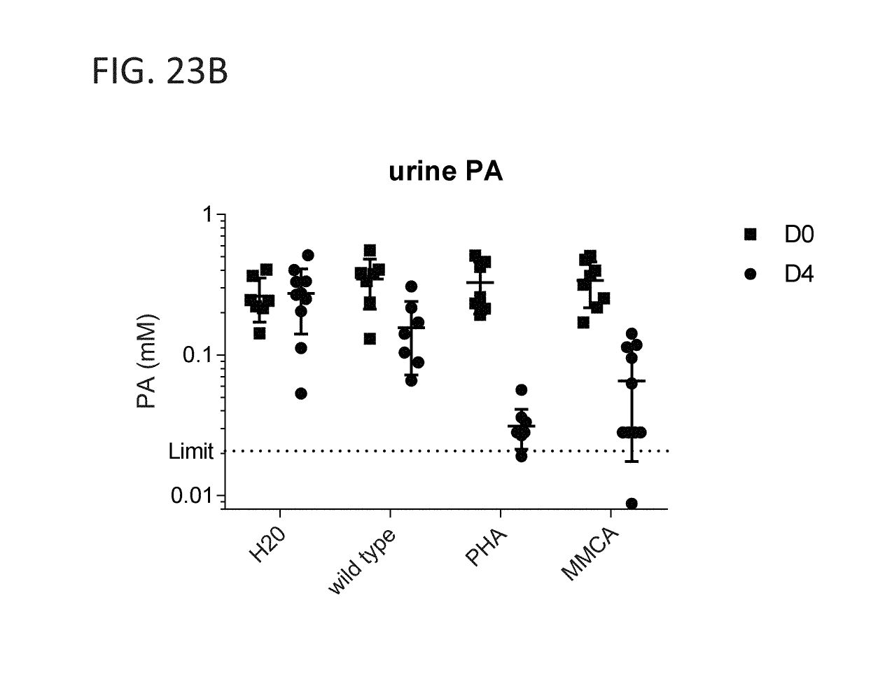

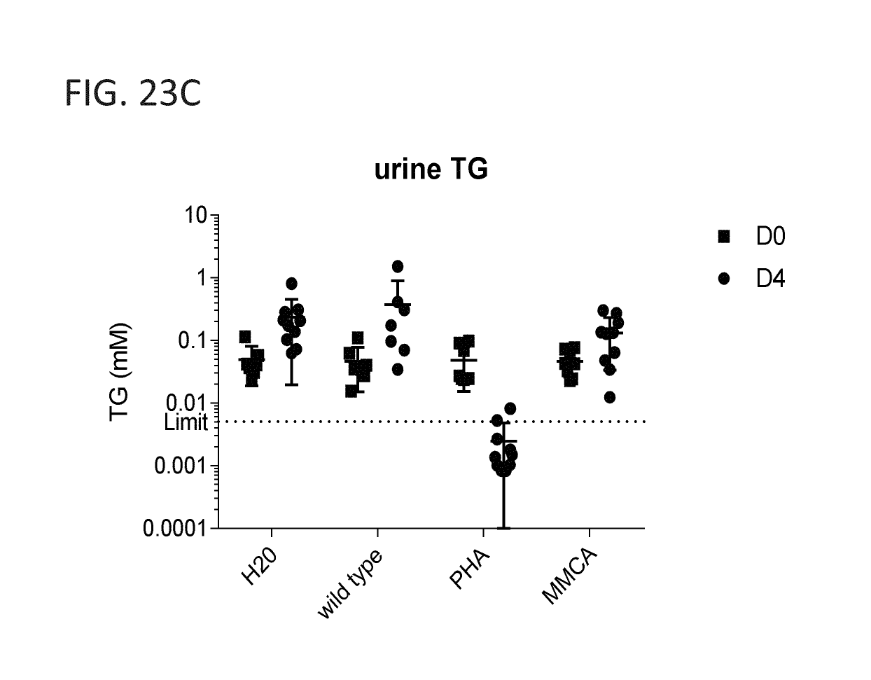

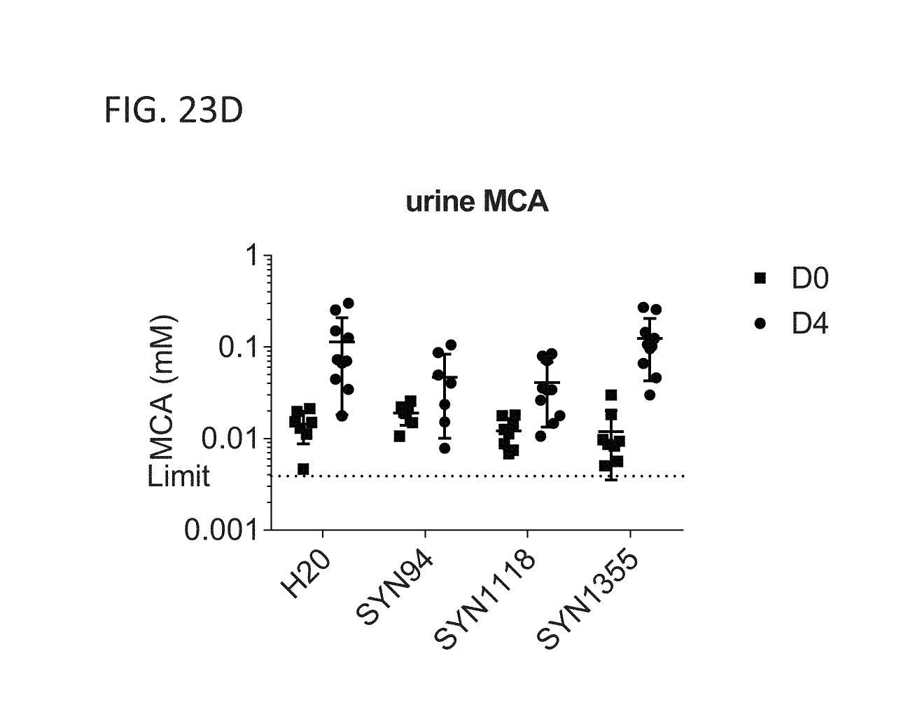

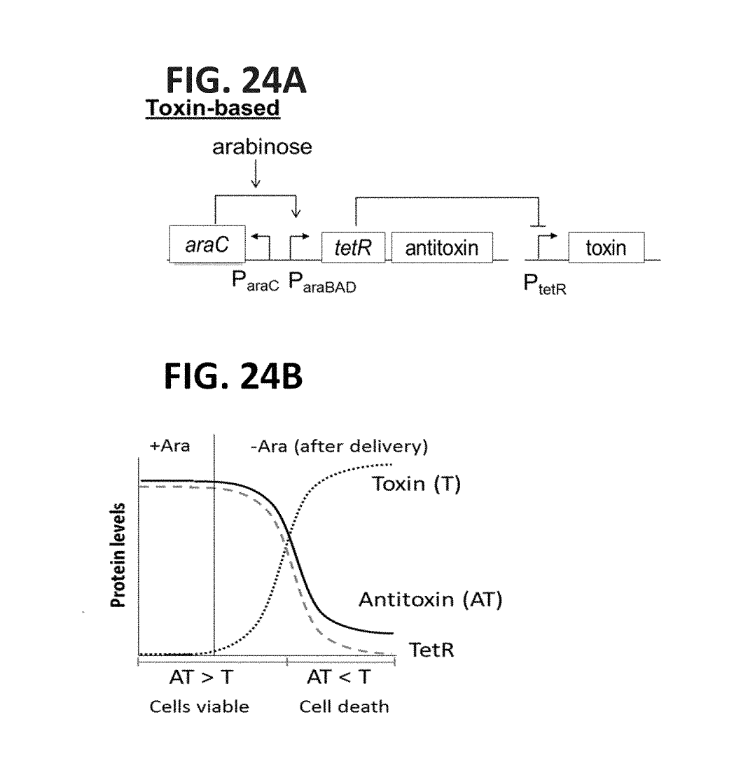

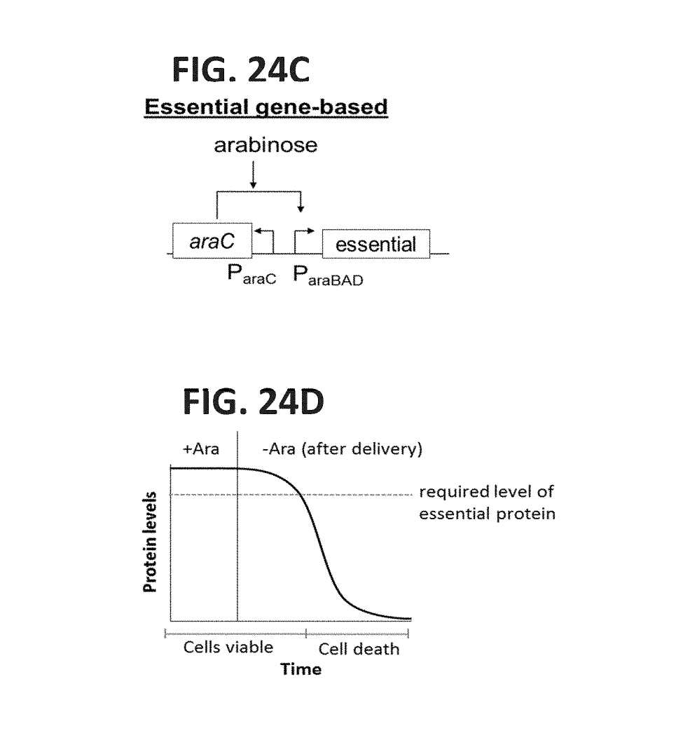

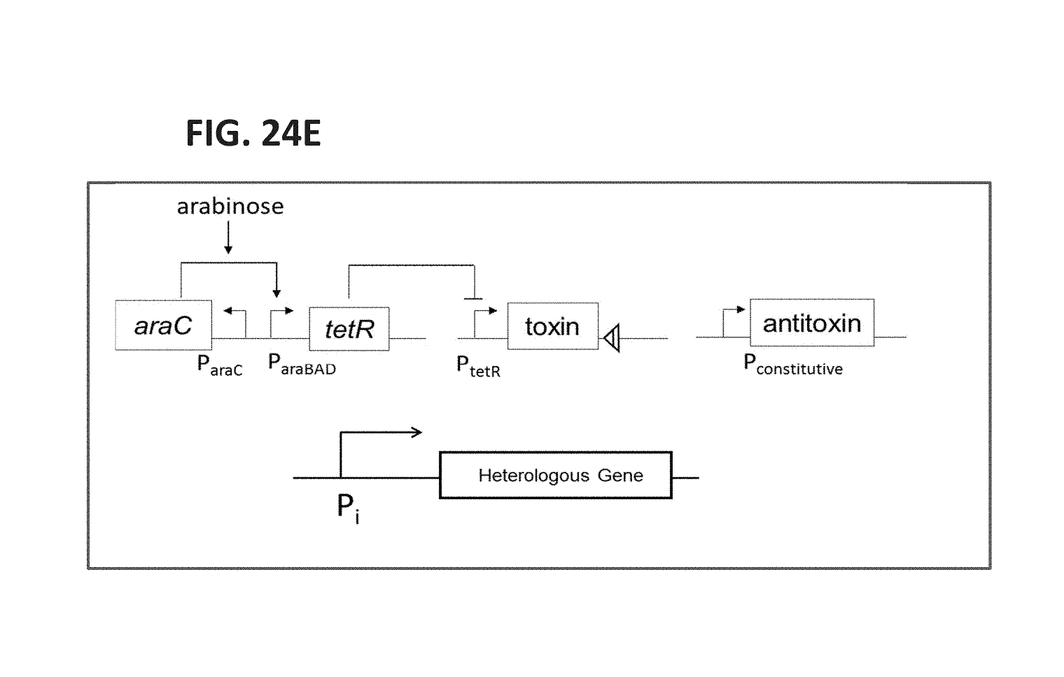

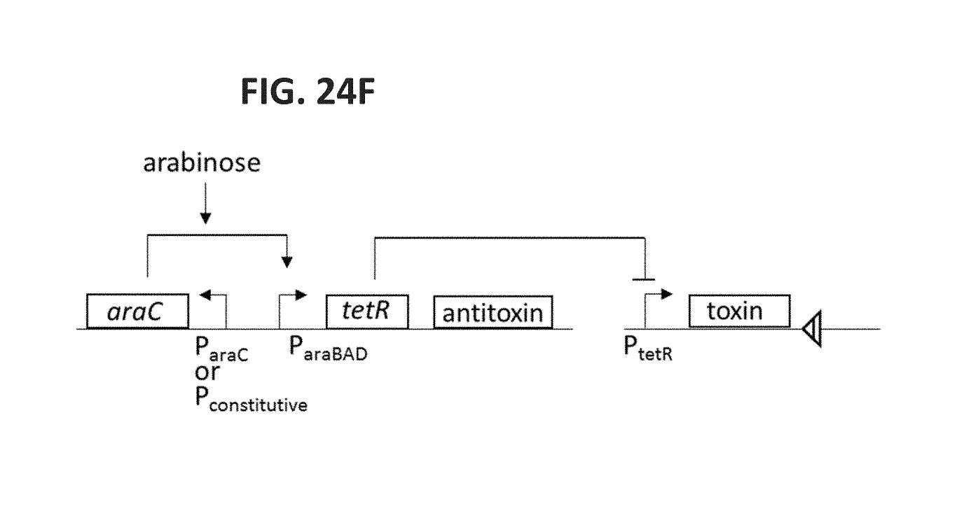

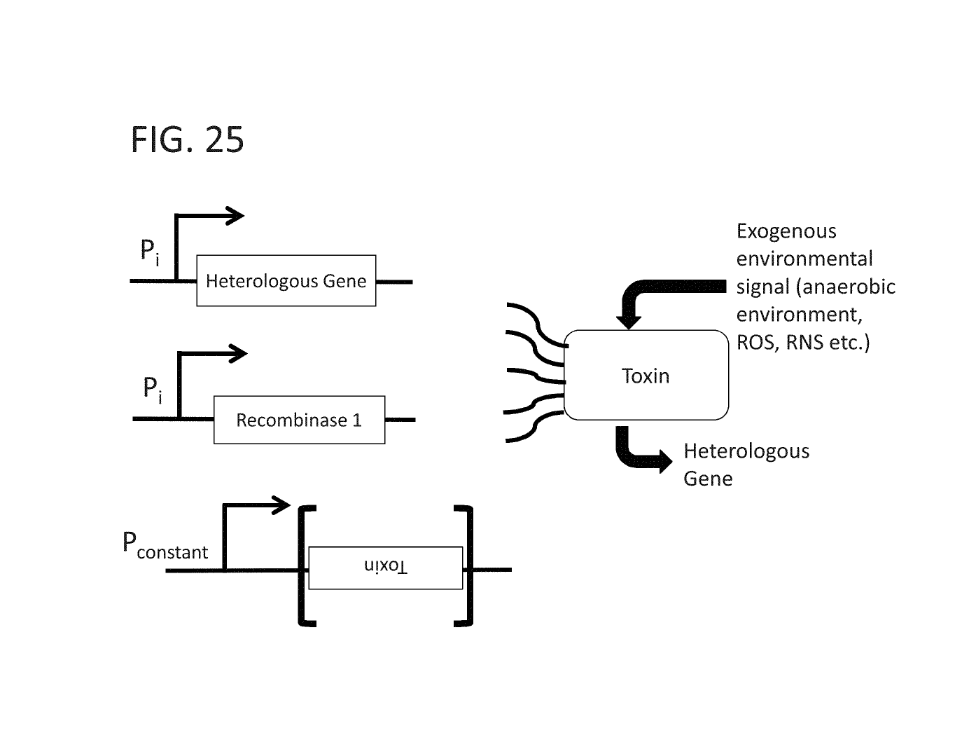

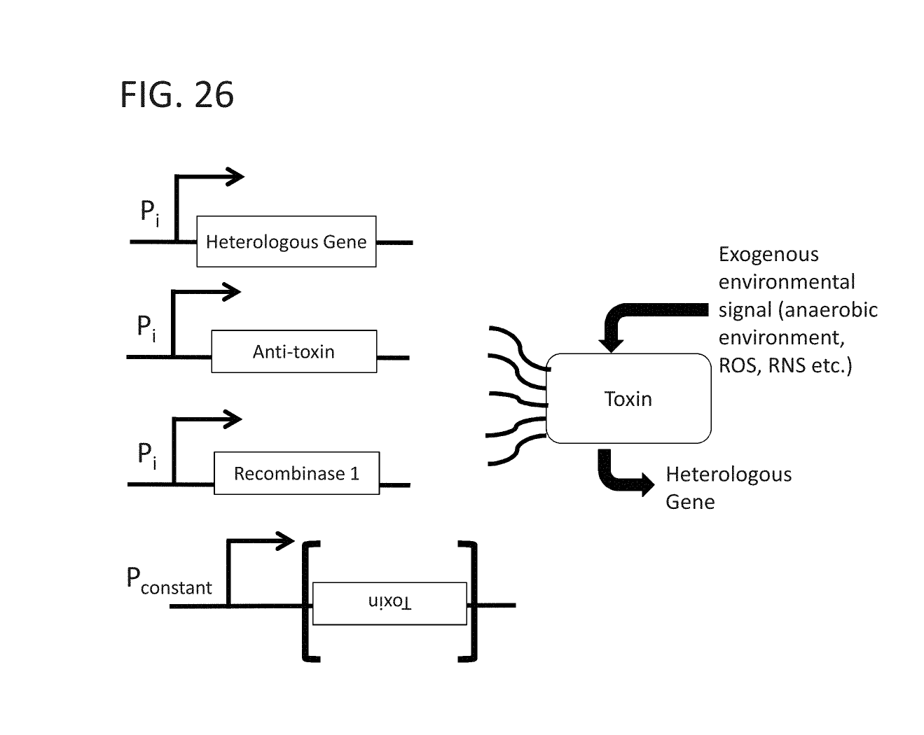

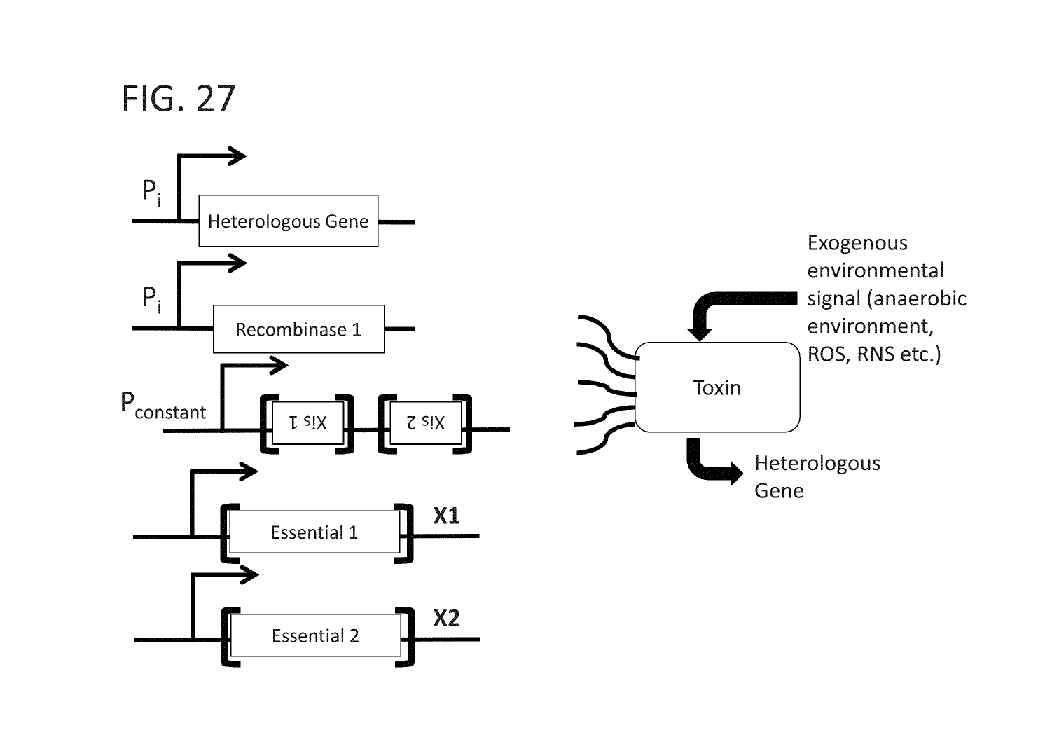

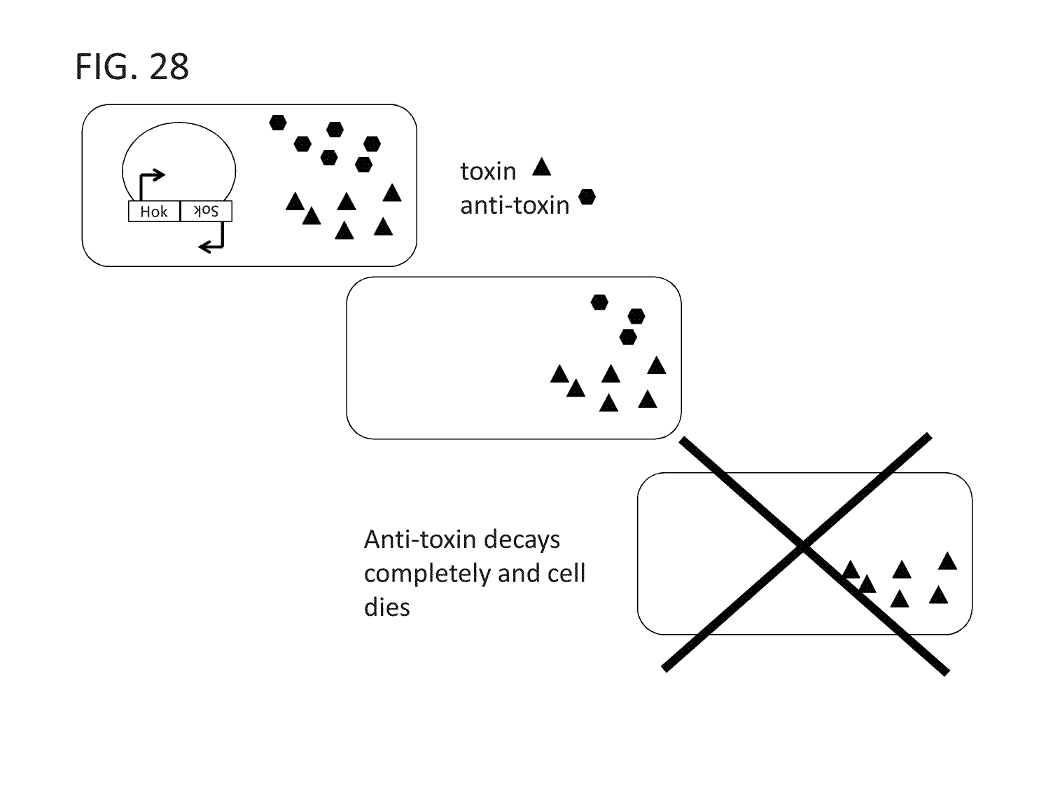

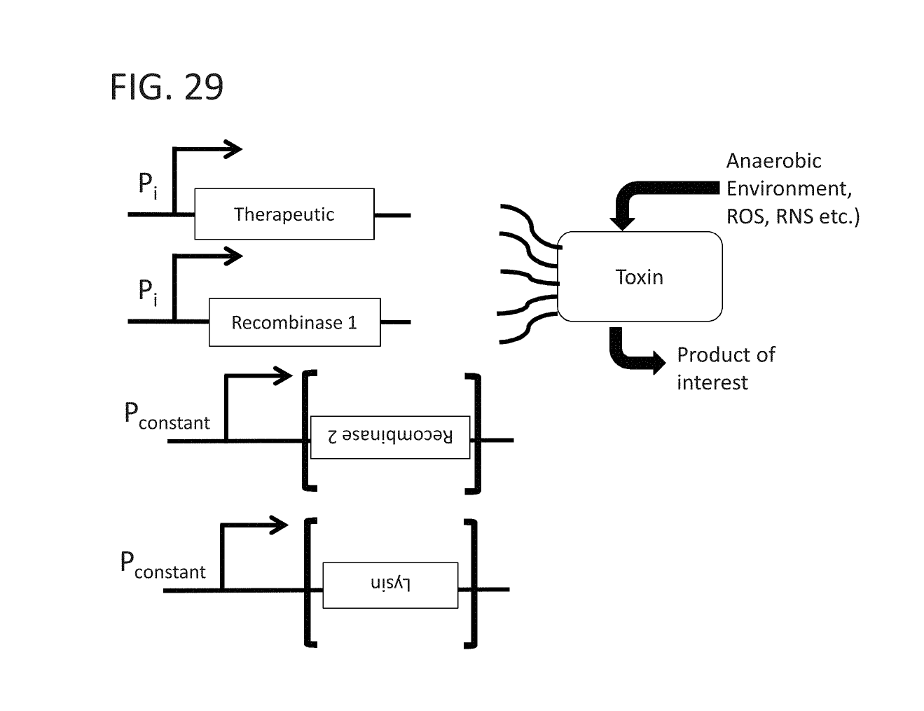

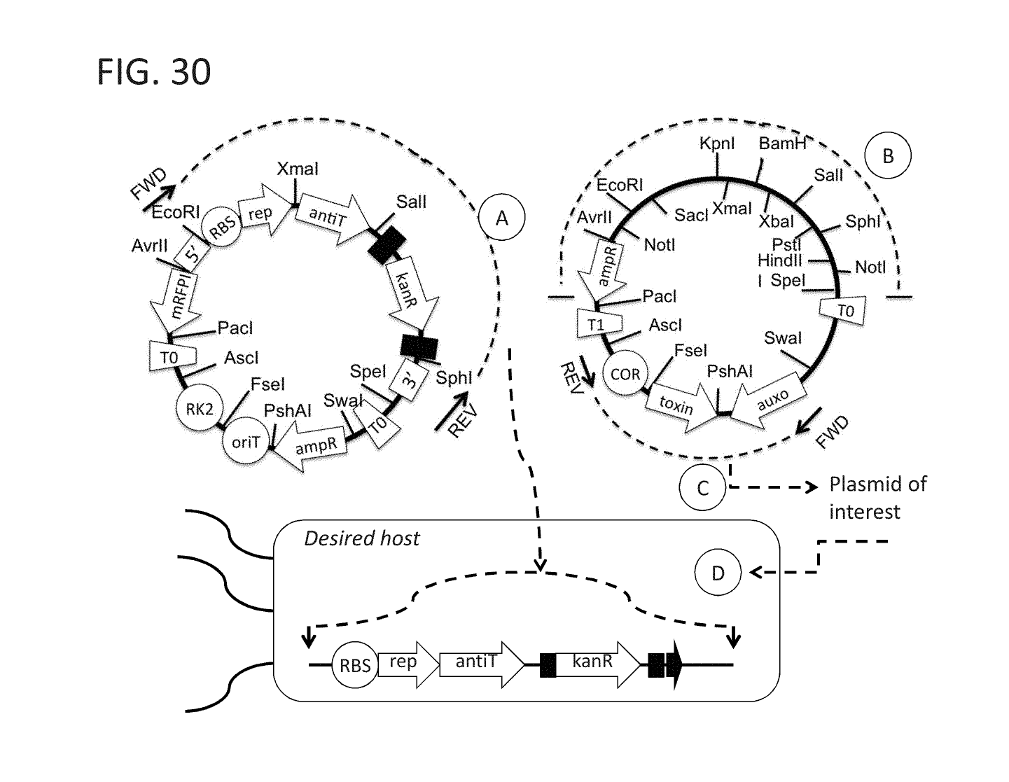

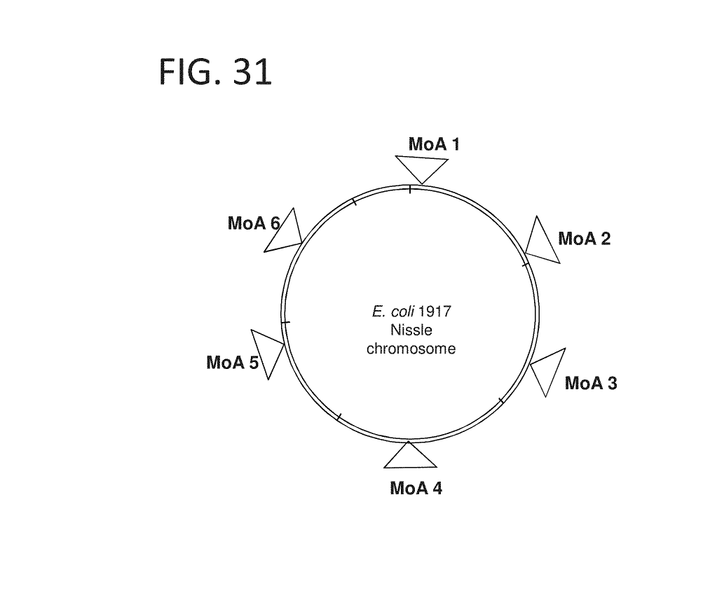

To prepare the PHA strain for this study, cultures of strains comprising the plasmid-based PHA pathway circuits, were grown overnight in LB and 50 ug/mL Ampicillin and then diluted 1:100 in LB, grown for 1.5 h aerobically, then ATC was added to cultures at a concentration of 100 ng/mL and cells were grown for an additional 2.5 hours. Prior to administration, cells were concentrated 200× and frozen (15% glycerol, 2 g/L glucose, in PBS). Cells were thawed on ice, and diluted in PBS to the appropriate concentration for dosing.
Results are shown in FIG. 23 and show that ratios of plasma propionylcarnitine to acetyl carnitine and urinary propionate were significantly reduced in both PHA and MMCA strains at 4 days post switch to high protein chow as compared to streptomycin resistant Nissle and H₂O controls. Urine triglycine was decreased in the PHA strain, but not the MMCA strain.

### Example 21. Diet-Induced Changes in Plasma and Urinary Biomarkers in Mut Ki/Ki and Mut Ko/Ki Mice Gavaged with PHA Pathway and MMCA Pathway Strains on High Protein Diet

The efficacy of two strains, one expressing PHA pathway genes (PHA), and the other expressing MMCA pathway genes (MMCA) in vivo was assessed using a mouse model of methylmalonic acidemia. Transgenic knock in (Mutki/ki) mice based on a Mut allele found in human patients (Forny et al., 2016) and a Mutko/ko mice resulting from a cross of Mutki/ki mice with Mut−/− mice were used as methylmalonic acidemia models. A high protein (HP) challenge and a precursor enriched (PE) diet in these models lead to metabolic crisis, which can be partially rescued by cobalamin.

Both strains used in the study are plasmid based strains expressing the pathway genes under the control of tetracycline inducible promoters (as described in Example 20). The PHA strain is described e.g., in Example 9 and FIG. 10C and FIG. 11 and elsewhere herein. The MMCA strain is described, e.g., in Example 12 and FIG. 15C, FIG. 16A, and FIG. 16B and elsewhere herein.

On day −7, animals (8 week old Mutki/ki and Mutki/ko mice) are placed on normal chow and water. Normal chow contains isoleucine at 10 g/Kg; valine at 12 g/Kg; and threonine at 7.6 g/Kg. Cobalamin control groups are injected with 0.3 ug hydroxocobalamin i.p (n=20). On day 1, animals are randomized into treatment groups. Mice are bled and urine is collected (T=0) to obtain baseline plasma and urine biomarker levels. Mice are grouped as follows: Group 1: H2O, HP (n=10); Group 2: wild type Nissle with streptomycin resistance, HP (n=10); Group 3: PHA strain, HP (n=10); Group 4: MMCA strain, HP (n=10). Group 5: H2O, PE (n=10); Group 6: wild type Nissle with streptomycin resistance (n=10), PE; Group 7: PHA strain, PE (n=10); Group 8: MMCA strain, PE (n=10), Group 9: cobalamin, HP (n=10); Group 10: cobalamin, PE (n=10). Group 11: H2O, NC (n=10); Group 12: wild type Nissle with streptomycin resistance, NC (n=10); Group 13: PHA strain, NC (n=10); Group 14: MMCA strain, NC (n=10). Mice are placed on high protein (HP) chow (Groups 1-4 and Group 9) or precursor enriched (PE) chow (Groups 5-8 and Group 10) as described in Forny et al. HP chow contains 35 g/Kg, 42 g/Kg and 27 g/Kg of isoleucine, valine and threonine, respectively. PE chow contains 70 g/Kg, 84 g/Kg and 53 g/Kg of isoleucine, valine and threonine, respectively. For the PE diet, leucine (19 g/kg, 119%) was enriched since its uptake might compete with the uptake of the other amino acids which are increased in the diet and cystine was increased (3.5 g/kg, 700%) to elevate the overall sulfur content.

For Groups 2, 3 and 4, 6, 7, and 8, mice are gavaged with 10e10 CFU/dose in 100 unclose. Group 1 and Group 5 are dosed with 100 ul H2O. For cobalamin rescue (Group 9 and Group 10), mice are injected with 0.3 ug hydroxocobalamin i.p. on day one and each following day throughout the study.

On days 2 through 5, mice are dosed twice daily with 100 ul bacteria (10e10 CFUs/dose) or H₂O (Group 1). On day 3 and 5, mice are dosed once with 100 ul bacteria (10e10 CFUs/dose) or water and animals are weighed and changes in weight are analyzed, blood is drawn and urine is collected at 4 hours post dose for LC/MS analysis. On day 5 animals are sacrificed and the brain, liver and kidney are removed and the weight of the brain normalized to body weight is tabulated. Levels of MMA, propionic acid, and MCA in blood and urine are measured. Blood C3/C2 ratios and ammonia levels are measured. MMA and 2-MC levels in brain, kidney and liver are measured as described in Forny et el.

To prepare the MMCA strain for this study, cultures comprising the two plasmid based MMCA pathway circuits, are grown overnight in LB and 50 ug/mL ampicillin and then diluted 1:100 in LB. The cells are grown with shaking (250 rpm) to early log phase with the appropriate antibiotics (1.5 h). Anhydrous tetracycline (ATC, 100 ng/ml) and arabinose (10 mM) is added to cultures to induce expression of the constructs, and bacteria are grown for another 2.5 hours. Prior to administration, cells are concentrated 200× and frozen (15% glycerol, 2 g/L glucose, in PBS). Cells are thawed on ice, and diluted in PBS to the appropriate concentration for dosing.

To prepare the PHA strain for this study, cultures of strains comprising the plasmid-based PHA pathway circuits, are grown overnight in LB and 50 ug/mL Ampicillin and then diluted 1:100 in LB, grown for 1.5 h aerobically, then ATC is added to cultures at a concentration of 100 ng/mL and cells are grown for an additional 2.5 hours. Prior to administration, cells are concentrated 200× and frozen (15% glycerol, 2 g/L glucose, in PBS). Cells are thawed on ice, and diluted in PBS to the appropriate concentration for dosing.

### Example 22. Diet-Induced Changes in Plasma and Urinary Biomarkers in PCCAA138T Hypomorph Mice Gavaged with PHA Pathway and MMCA Pathway Strains Induced Under Low Oxygen Conditions on Normal Chow and on High Protein Diet

### Strain Generation

In order to assess the efficacy of strains in which the genetic circuits are expressed under conditions present in the gut, e.g., low oxygen conditions, constructs are generated in which the tet promoters in the plasmids described in Examples 19 and 20 are replaced with a low oxygen promoter, e.g., a FNR promoter. First, strains are generated in which the constructs are expressed from plasmids. Next strains are generated in which one or more circuits are integrated into the bacterial chromosome at one or more sites, e.g., as described in FIG. 32 and elsewhere herein, according to methods described herein (e.g., Example 5) and known in the art. These strains are then first tested in vitro for propionate consumption activity and then tested for in vivo efficacy in the PCCAA138T hypomorph model.

### In Vitro Testing

For in vitro testing, cultures of E. coli Nissle comprising either the prpE-phaBCA circuit or the prpE-accAB and mmcE-mutAB circuits driven by the FNR promoter (either on a plasmid or as one or more copies inserted into the bacterial chromosome) and cultures of wild type control Nissle are grown overnight and then diluted 1:200 in LB. All strains are grown for 1.5 hrs before cultures are placed in a Coy anaerobic chamber supplying 90% N₂, 5% CO₂, and 5% H₂. After 4 hrs of induction, bacteria are pelleted, washed in PBS, and resuspended in 1 mL M9 medium supplemented with glucose (0.2%) and propionate (2-8 mM) at a concentration of ˜10⁹ cfu/ml bacteria. Aliquots are collected at 0 hrs, 1.5 hr, 3 hrs, and 4.5 hrs for propionate quantification as described herein.

### In Vivo Testing (PCCAA138T Model)

Next the activity of the strains is tested in vivo using the PCCAA138T hypomorph mice model on normal chow and high protein chow. With exception of the preparation of cells, the studies are essentially carried out as described in Example 19 and 20.

To prepare the cells for these studies, cells are diluted 1:100 in LB (2 L), grown for 1.5 h aerobically, then shifted to the anaerobe chamber for 4 hours. Prior to administration, cells are concentrated 200× and frozen (15% glycerol, 2 g/L glucose, in PBS). Cells are thawed on ice, and diluted in PBS to the appropriate concentration for dosing.

In Vivo Testing (Mutki/Ki and Mutki/Ko Models)

Next the activity of the strains is tested in vivo using the Mutki/ki and Mutki/ko models on normal chow, high protein chow, and precursor enriched chow. With exception of the preparation of cells, the studies are essentially carried out as described in Example 21.

To prepare the cells for these studies, cells are diluted 1:100 in LB (2 L), grown for 1.5 h aerobically, then shifted to the anaerobe chamber for 4 hours. Prior to administration, cells are concentrated 200× and frozen (15% glycerol, 2 g/L glucose, in PBS). Cells are thawed on ice, and diluted in PBS to the appropriate concentration for dosing.

### Testing of Additional Strains

In additional studies, the utility of other constitutive or inducible promoters is tested. In order to test the efficacy of strains in which the genetic circuits are expressed under various inducible and constitutive s promoters. Strains are generated in which the PHA and MMCA circuits are expressed under the control of these promoters, either from a plasmid or from one or more copies which are integrated into the bacterial chromosome. If two operons are used, then each operon can be driven by a different promoter.

The strains are then induced and tested for in vitro activity. In brief, cultures of E. coli Nissle comprising either the prpE-phaBCA circuit or the prpE-accAB and mmcE-mutAB circuits driven by the inducible promoter(s) (either on a plasmid or as one or more copies inserted into the bacterial chromosome) and cultures of wild type control Nissle are grown overnight and then diluted 1:200 in LB. All strains are grown for 1.5 to 2 hours and then cultures are induced, e.g., for 1 to 5 hrs, according to conditions required for induction of the promoter(s) driving expression of the constructs. Subsequently, bacteria are pelleted, washed in PBS, and resuspended in 1 mL M9 medium supplemented with glucose (0.2%) and propionate (2-8 mM) at a concentration of ˜10⁹ cfu/ml bacteria. Aliquots are collected at 0 hrs, 1.5 hr, 3 hrs, and 4.5 hrs for propionate quantification as described herein.

For in vivo activity, the PCCAA138T hypomorph mice model on normal chow and high protein chow can be used. With exception of the preparation of cells, the studies are essentially carried out as described in Example 19 and 20.

To prepare the cells for these studies, cells are diluted 1:100 in LB (2 L), grown for 1 to 2 h, then induced according to conditions required for induction of the promoter(s) driving expression of the constructs for 1-5 hours. Prior to administration, cells are concentrated 200× and frozen (15% glycerol, 2 g/L glucose, in PBS). Cells are thawed on ice, and diluted in PBS to the appropriate concentration for dosing.

### Example 23. Diet-Induced Changes in Plasma and Urinary Biomarkers in PCCAA138T Hypomorph, Mutki/Ki and Mutki/Ko Mice Gavaged with Various Genetically Engineered Strains Induced by Tetracycline or Under Low Oxygen Conditions on Normal Chow and on High Protein Diet

### Strain Generation

A number of additional strains are tested in vivo and in vivo. Additional PHA pathway strains with two plasmids were generated as shown in FIG. 14A, FIG. 14B. FIGS. 14C, and 14D.

Additional strains are generated in which the two PHA constructs are integrated into the genome. Further strains are generated (both plasmid based and integrated strains) in which the tetracycline and arabinose promoters are replaced with a promoter induced under conditions present in the gut, i.e., low oxygen conditions. Specifically, a FNR promoter is used. Strains comprising the two FNR-PHA constructs are tested in vitro as described in Example 22.

For in vitro testing, cultures of E. coli Nissle comprising the PHA circuits n by the FNR promoter (either on 2 plasmids or as one or more copies inserted into the bacterial chromosome) and cultures of wild type control Nissle are grown overnight and then diluted 1:200 in LB. All strains are grown for 1.5 hrs before cultures are placed in a Coy anaerobic chamber supplying 90% N₂, 5% CO₂, and 5% H₂. After 4 hrs of induction, bacteria are pelleted, washed in PBS, and resuspended in 1 mL M9 medium supplemented with glucose (0.2%) and propionate (2-8 mM) at a concentration of ˜10⁹ cfu/ml bacteria. Aliquots are collected at 0 hrs, 1.5 hr, 3 hrs, and 4.5 hrs for propionate quantification as described herein.

### In Vivo Testing PCCAA138T Model

Next the activity of the strains is tested in vivo using the PCCAA138T hypomorph mice model on normal chow and high protein chow. With exception of the preparation of cells, the studies are essentially carried out as described in Example 19 and 20.

To prepare the cells comprising the arabinose and tetracycline driven constructs, cells are diluted 1:100 in LB (2 L), grown for 1-2 hours. ATC (100 ng/mL) is added to induce the tet-construct gene cassette and arabinose is added at a concentration of 10 mM to induce the second plasmid and cells are grown with for 2 hours. Prior to administration, cells are concentrated 200× and frozen (15% glycerol, 2 g/L glucose, in PBS). Cells are thawed on ice, and diluted in PBS to the appropriate concentration for dosing.

To prepare the cells comprising the FNR driven constructs, cells are diluted 1:100 in LB (2 L), grown for 1.5 h aerobically, then shifted to the anaerobe chamber for 4 hours. Prior to administration, cells are concentrated 200× and frozen (15% glycerol, 2 g/L glucose, in PBS). Cells are thawed on ice, and diluted in PBS to the appropriate concentration for dosing.

In vivo testing (Mutki/ki and Mutki/ko models)

Next the activity of the strains is tested in vivo using the Mutki/ki and Mutki/ko models on normal chow, high protein chow, and precursor enriched chow. With exception of the preparation of cells, the studies are essentially carried out as described in Example 21.

The cells are prepared according to the same protocols as described in the previous section for the PCCAA138T model study.

### Testing of Additional Promoters

In additional studies, the utility of other constitutive or inducible promoters is tested. In order to test the efficacy of strains in which the genetic circuits are expressed under various inducible promoters, strains are generated in which the PHA circuits are expressed under the control of these promoters, either from a plasmid or from one or more copies which are integrated into the bacterial chromosome. Each operon can be driven by a different promoter.

The strains are then induced and tested for in vitro activity. In brief, cultures of E. coli Nissle comprising the PHA circuits driven by the inducible promoter(s) (either on a plasmid or as one or more copies inserted into the bacterial chromosome) and cultures of wild type control Nissle are grown overnight and then diluted 1:200 in LB. All strains are grown for 1.5 to 2 hours and then cultures are induced, e.g., for 1 to 5 hrs, according to conditions required for induction of the promoter(s) driving expression of the constructs. Subsequently, bacteria are pelleted, washed in PBS, and resuspended in 1 mL M9 medium supplemented with glucose (0.2%) and propionate (2-8 mM) at a concentration of ˜10⁹ cfu/ml bacteria. Aliquots are collected at 0 hrs, 1.5 hr, 3 hrs, and 4.5 hrs for propionate quantification as described herein.

For in vivo activity, the PCCAA138T hypomorph mice model on normal chow and high protein chow can be used. With exception of the preparation of cells, the studies are essentially carried out as described in Example 19 and 20. To prepare the cells for these studies, cells are diluted 1:100 in LB (2 L), grown for 1 to 2 h, then induced according to conditions required for induction of the promoter(s) driving expression of the constructs for 1-5 hours. Prior to administration, cells are concentrated 200× and frozen (15% glycerol, 2 g/L glucose, in PBS). Cells are thawed on ice, and diluted in PBS to the appropriate concentration for dosing.

The activity of the strains is also tested in vivo using the Mutki/ki and Mutki/ko models on normal chow, high protein chow, and precursor enriched chow. With exception of the preparation of cells, the studies are essentially carried out as described in Example 21.

The cells are prepared according to the same protocols as described in the previous paragraph for the PCCAA138T model study.

### Additional Strains

Additional strains are tested essentially according to the three steps described in this and other examples (1) strain generation, plasmid based and integrated strains (2) in vitro testing, (3) in vivo testing in the PCCAA138T hypomorph model and (4) in vivo testing in the Mut wt/ki and Mut ko/ki models

Additional strains include MMCA pathway strains that further comprise a gene sequence(s) for the expression of sucE1 succinate exporter (e.g., from Corynebacterium glutamicum) and/or the native Nissle succinate exporter dcuC, e.g., as shown in FIG. 17B. Expression of one or both 1 succinate exporter(s) is combined with any of the MMCA pathway strains, described in Example 19, Example 20, Example 21, or Example 23, and succinate exporter(s) and MMCA pathway cassettes are under the control of one or more of the promoters described in these examples. Testing is conducted as described above.

Other strains generated and tested are strains based on the 2-methylcitrate pathway described herein, e.g., comprising one or more gene cassette(s) comprising prpB, prpC, prpD, and prpE, e.g., as shown in FIG. 20B. 2-methyl citrate cassettes are under the control of one or more of the promoters described in Example 19, Example 20, Example 21, or Example 23.

Yet other strains generated and tested using various inducible systems are HA strains shown in FIG. 21A (increased PhaC), FIG. 21B (PHA strain with thyA auxotrophy or dapA auxotrophy), FIG. 21C (PHA pathway with thyA or dapA auxotroph), FIG. 21D (MMCA strain with succinate exporter), FIG. 21E (combination of MMCA and PHA circuits in one strain), FIG. 21F (MMCA pathway and a MatB circuit), FIG. 21 (combination of MMCA and PHA and MatB circuits in one strain), and MatB circuits in combination with 2MC circuits. IN these strains the pathway cassettes are under the control of one or more of the promoters described in Example 19, Example 20, Example 21, or Example 23.

### Example 24. Methylmalonic Acid and Methylcitric Acid Quantification in Bacterial Supernatant and Plasma by LC-MS/MS

### Sample Preparation

Methylmalonic acid (MMA) and Methylcitric acid (2-MCA) stock (10 mg/mL) is prepared in DMSO and aliquot in 1.5 mL microcentrifuge tubes (100 μL). Standards (250, 100, 20, 4, 0.8, 0.16, 0.032 μg/mL) of each are prepared in water. Sample (10 μL) (and standards) are mixed with 90 μL of ACN/H₂O (60:30, v/v) containing 1 μg/mL of MCA-d₃ in the final solution) in a V-bottom 96-well plate. The plate is heat sealed with a AlumASeal foil, mixed well, and centrifuged at 4000 rpm for 5 min 10 μL of the solution is transferred in a round-bottom 96-well plate, and 90 uL if 0.1% formic acid in water is added to the sample. The plate is heat-sealed with a ClearASeal sheet and mixed well.

### LC-MS/MS Method

2-MCA and MMA are measured by liquid chromatography coupled to tandem mass spectrometry (LC-MS/MS) using a Thermo TSQ Quantum Max triple quadrupole mass spectrometer. Table 43, Table 44, and Table 45 provides the summary of the LC-MS/MS method.

TABLE 43
HPLC Method

| | |
|---|---|
| Column | C18 column, 3 μm (100 × 2.1 mm) |
| Mobile Phase A | 99.9% H2O, 0.1% Formic Acid |
| Mobile Phase B | Methanol |
| Injection volume | 10 uL |

TABLE 44
HPLC Method

| Time (min) | Flow Rate | A % | B % |
|---|---|---|---|
| 0 | 500 | 90 | 10 |
| 0.5 | 500 | 90 | 10 |
| 2.0 | 500 | 3 | 97 |
| 4.0 | 500 | 3 | 97 |
| 4.01 | 500 | 10 | 10 |
| 4.25 | 500 | 10 | 10 |

TABLE 45
Tandem Mass Spectrometry:

| | |
|---|---|
| Ion Source | HESI-II |
| Polarity | Negative |

SRM transitions:

| | |
|---|---|
| 2-MCA: | 205.1/125.3 |
| MMA: | 117.3/73.4 |
| 2-MCA-d3: | 208.1/128.3 |

### Example 25. Generation of ΔThyA

An auxotrophic mutation causes bacteria to die in the absence of an exogenously added nutrient essential for survival or growth because they lack the gene(s) necessary to produce that essential nutrient. In order to generate genetically engineered bacteria with an auxotrophic modification, the thyA, a gene essential for oligonucleotide synthesis was deleted. Deletion of the thyA gene in E. coli Nissle yields a strain that cannot form a colony on LB plates unless they are supplemented with thymidine.

A thyA::cam PCR fragment was amplified using 3 rounds of PCR as follows. Sequences of the primers used at a 100 um concentration are found in Table 46.

TABLE 46
Primer Sequences

| | | | SEQ ID |
|---|---|---|---|
| Name | Sequence | Description | NO |
| SR36 | tagaactgatgcaaaaagtgctcgacgaaggcacacagaTGTGTAGG | Round 1: binds | SEQ ID |
| | CTGGAGCTGCTTC | on pKD3 | NO: 59 |
| SR38 | gtttcgtaattagatagccaccggcgctttaatgcccggaCATATGAAT | Round 1: binds | SEQ ID |
| | ATCCTCCTTAG | on pKD3 | NO: 60 |
| SR33 | caacacgtttcctgaggaaccatgaaacagtatttagaactgatgcaaaaag | Round 2: binds to | SEQ ID |
| | | round 1 PCR | NO: 61 |
| | | product | |
| SR34 | cgcacactggcgtcggctctggcaggatgtttcgtaattagatagc | Round 2: binds to | SEQ ID |
| | | round 1 PCR | NO: 62 |
| | | product | |
| SR43 | atatcgtcgcagcccacagcaacacgtttcctgagg | Round 3: binds to | SEQ ID |
| | | round 2 PCR | NO: 63 |
| | | product | |
| SR44 | aagaatttaacggagggcaaaaaaaaccgacgcacactggcgtcggc | Round 3: binds to | SEQ ID |
| | | round 2 PCR | NO: 64 |
| | | product | |

For the first PCR round, 4×50 ul PCR reactions containing ing pKD3 as template, 25 ul 2×phusion, 0.2 ul primer SR36 and SR38, and either 0, 0.2, 0.4 or 0.6 ul DMSO were brought up to 50 ul volume with nuclease free water and amplified under the following cycle conditions:

step1: 98 c for 30 s

step2: 98 c for 10 s

step3: 55 c for 15 s

step 4: 72 c for 20 s

repeat step 2-4 for 30 cycles

step5: 72 c for 5 min

Subsequently, 5 ul of each PCR reaction was run on an agarose gel to confirm PCR product of the appropriate size. The PCR product was purified from the remaining PCR reaction using a Zymoclean gel DNA recovery kit according to the manufacturer's instructions and eluted in 30 ul nuclease free water.

For the second round of PCR, 1 ul purified PCR product from round 1 was used as template, in 4×50 ul PCR reactions as described above except with 0.2 ul of primers SR33 and SR34. Cycle conditions were the same as noted above for the first PCR reaction. The PCR product run on an agarose gel to verify amplification, purified, and eluted in 30 ul as described above.

For the third round of PCR, 1 ul of purified PCR product from round 2 was used as template in 4×50 ul PCR reactions as described except with primer SR43 and SR44. Cycle conditions were the same as described for rounds 1 and 2. Amplification was verified, the PCR product purified, and eluted as described above. The concentration and purity was measured using a spectrophotometer. The resulting linear DNA fragment, which contains 92 bp homologous to upstream of thyA, the chloramphenicol cassette flanked by frt sites, and 98 bp homologous to downstream of the thyA gene, was transformed into a E. coli Nissle 1917 strain containing pKD46 grown for recombineering. Following electroporation, 1 ml SOC medium containing 3 mM thymidine was added, and cells were allowed to recover at 37 C for 2 h with shaking. Cells were then pelleted at 10,000×g for 1 minute, the supernatant was discarded, and the cell pellet was resuspended in 100 ul LB containing 3 mM thymidine and spread on LB agar plates containing 3 mM thy and 20 ug/ml chloramphenicol. Cells were incubated at 37 C overnight. Colonies that appeared on LB plates were restreaked. + cam 20 ug/ml + or − thy 3 mM. (thyA auxotrophs will only grow in media supplemented with thy 3 mM).

Next the antibiotic resistance was removed with pCP20 transformation. pCP20 has the yeast Flp recombinase gene, FLP, chloramphenicol and ampicillin resistant genes, and temperature sensitive replication. Bacteria were grown in LB media containing the selecting antibiotic at 37° C. until OD600=0.4-0.6. 1 mL of cells were washed as follows: cells were pelleted at 16,000×g for 1 minute. The supernatant was discarded and the pellet was resuspended in 1 mL ice-cold 10% glycerol. This wash step was repeated 3× times. The final pellet was resuspended in 70 ul ice-cold 10% glycerol. Next, cells were electroporated with ing pCP20 plasmid DNA, and 1 mL SOC supplemented with 3 mM thymidine was immediately added to the cuvette. Cells were resuspended and transferred to a culture tube and grown at 30° C. for 1 hours. Cells were then pelleted at 10,000×g for 1 minute, the supernatant was discarded, and the cell pellet was resuspended in 100 ul LB containing 3 mM thymidine and spread on LB agar plates containing 3 mM thy and 100 ug/ml carbenicillin and grown at 30° C. for 16-24 hours. Next, transformants were colony purified non-selectively (no antibiotics) at 42° C.

To test the colony-purified transformants, a colony was picked from the 42° C. plate with a pipette tip and resuspended in 10 μL LB. 3 μL of the cell suspension was pipetted onto a set of 3 plates: Cam, (37° C.; tests for the presence/absence of CamR gene in the genome of the host strain), Amp, (30° C., tests for the presence/absence of AmpR from the pCP20 plasmid) and LB only (desired cells that have lost the chloramphenicol cassette and the pCP20 plasmid), 37° C. Colonies were considered cured if there is no growth in neither the Cam or Amp plate, picked, and re-streaked on an LB plate to get single colonies, and grown overnight at 37° C.

Subsequently, 5 ul of each PCR reaction was run on an agarose gel to confirm PCR product of the appropriate size. The PCR product was purified from the remaining PCR reaction using a Zymoclean gel DNA recovery kit according to the manufacturer's instructions and eluted in 30 ul nuclease free water.

For the second round of PCR, 1 ul purified PCR product from round 1 was used as template, in 4×50 ul PCR reactions as described above except with 0.2 ul of primers SR33 and SR34. Cycle conditions were the same as noted above for the first PCR reaction. The PCR product run on an agarose gel to verify amplification, purified, and eluted in 30 ul as described above.

For the third round of PCR, 1 ul of purified PCR product from round 2 was used as template in 4×50 ul PCR reactions as described except with primer SR43 and SR44. Cycle conditions were the same as described for rounds 1 and 2. Amplification was verified, the PCR product purified, and eluted as described above. The concentration and purity was measured using a spectrophotometer. The resulting linear DNA fragment, which contains 92 bp homologous to upstream of thyA, the chloramphenicol cassette flanked by frt sites, and 98 bp homologous to downstream of the thyA gene, was transformed into a E. coli Nissle 1917 strain containing pKD46 grown for recombineering. Following electroporation, 1 ml SOC medium containing 3 mM thymidine was added, and cells were allowed to recover at 37 C for 2 h with shaking. Cells were then pelleted at 10,000×g for 1 minute, the supernatant was discarded, and the cell pellet was resuspended in 100 ul LB containing 3 mM thymidine and spread on LB agar plates containing 3 mM thy and 20 ug/ml chloramphenicol. Cells were incubated at 37 C overnight. Colonies that appeared on LB plates were restreaked. + cam 20 ug/ml + or − thy 3 mM. (thyA auxotrophs will only grow in media supplemented with thy 3 mM).

Next, the antibiotic resistance was removed with pCP20 transformation. pCP20 has the yeast Flp recombinase gene, FLP, chloramphenicol and ampicillin resistant genes, and temperature sensitive replication. Bacteria were grown in LB media containing the selecting antibiotic at 37° C. until OD600=0.4-0.6. 1 mL of cells were washed as follows: cells were pelleted at 16,000×g for 1 minute. The supernatant was discarded and the pellet was resuspended in 1 mL ice-cold 10% glycerol. This wash step was repeated 3× times. The final pellet was resuspended in 70 ul ice-cold 10% glycerol. Next, cells were electroporated with ing pCP20 plasmid DNA, and 1 mL SOC supplemented with 3 mM thymidine was immediately added to the cuvette. Cells were resuspended and transferred to a culture tube and grown at 30° C. for 1 hours. Cells were then pelleted at 10,000×g for 1 minute, the supernatant was discarded, and the cell pellet was resuspended in 100 ul LB containing 3 mM thymidine and spread on LB agar plates containing 3 mM thy and 100 ug/ml carbenicillin and grown at 30° C. for 16-24 hours. Next, transformants were colony purified non-selectively (no antibiotics) at 42° C.

To test the colony-purified transformants, a colony was picked from the 42° C. plate with a pipette tip and resuspended in 10 μL LB. 3 μL of the cell suspension was pipetted onto a set of 3 plates: Cam, (37° C.; tests for the presence/absence of CamR gene in the genome of the host strain), Amp, (30° C., tests for the presence/absence of AmpR from the pCP20 plasmid) and LB only (desired cells that have lost the chloramphenicol cassette and the pCP20 plasmid), 37° C. Colonies were considered cured if there is no growth in neither the Cam or Amp plate, picked, and re-streaked on an LB plate to get single colonies, and grown overnight at 37° C.

In other embodiments, similar methods are used to create other auxotrophies, including, but not limited to, dapA.

### Example 26. Nitric Oxide-Inducible Reporter Constructs

ATC and nitric oxide-inducible reporter constructs were synthesized (Genewiz, Cambridge, Mass.). When induced by their cognate inducers, these constructs express GFP, which is detected by monitoring fluorescence in a plate reader at an excitation/emission of 395/509 nm, respectively. Nissle cells harboring plasmids with either the control, ATC-inducible Ptet-GFP reporter construct, or the nitric oxide inducible PnsrR-GFP reporter construct were first grown to early log phase (OD600 of about 0.4-0.6), at which point they were transferred to 96-well microtiter plates containing LB and two-fold decreased inducer (ATC or the long half-life NO donor, DETA-NO (Sigma)). Both ATC and NO were able to induce the expression of GFP in their respective constructs across a range of concentrations (FIG. 43); promoter activity is expressed as relative florescence units. An exemplary sequence of a nitric oxide-inducible reporter construct is shown. The bsrR sequence is bolded. The gfp sequence is underlined. The PnsrR (NO regulated promoter and RBS) is italicized. The constitutive promoter and RBS are boxed. These constructs, when induced by their cognate inducer, lead to high level expression of GFP, which is detected by monitoring fluorescence in a plate reader at an excitation/emission of 395/509 nm, respectively. Nissle cells harboring plasmids with either the ATC-inducible Ptet-GFP reporter construct or the nitric oxide inducible PnsrR-GFP reporter construct were first grown to early log phase (OD600=˜0.4-0.6), at which point they were transferred to 96-well microtiter plates containing LB and 2-fold decreases in inducer (ATC or the long half-life NO donor, DETA-NO (Sigma)). It was observed that both the ATC and NO were able to induce the expression of GFP in their respective construct across a wide range of concentrations. Promoter activity is expressed as relative florescence units.

TABLE 47
Nitric Oxide-inducible Reporter Construct
(SEQ ID NO: [[309]]322)

SEQ ID NO: [[309]]322
ttattatcgcaccgcaatcgggattttcgattcataaagcaggtcgtagg
tcggcttgttgagcaggtcttgcagcgtgaaaccgtccagatacgtgaaa
aacgacttcattgcaccgccgagtatgcccgtcagccggcaggacggcgt
aatcaggcattcgttgttcgggcccatacactcgaccagctgcatcggtt
cgaggtggcggacgaccgcgccgatattgatgcgttcgggcggcgcggcc
agcctcagcccgccgcctttcccgcgtacgctgtgcaagaacccgccttt
gaccagcgcggtaaccactttcatcaaatggcttttggaaatgccgtagg
tcgaggcgatggtggcgatattgaccagcgcgtcgtcgttgacggcggtg
tagatgaggacgcgcagcccgtagtcggtatgttgggtcagatacataca
acctccttagtacatgcaaaattatttctagagcaacatacgagccggaa
gcataaagtgtaaagcctggggtgcctaatgagttgagttgaggaattat
aacaggaagaaatattcctcatacgcttgtaattcctctatggttgttga
caattaatcatcggctcgtataatgtataacattcatattttgtgaattt
taaactctagaaataattttgtttaactttaagaaggagatatacatatg
gctagcaaaggcgaagaattgttcacgggcgttgttcctattttggttga
attggatggcgatgttaatggccataaattcagcgttagcggcgaaggcg
aaggcgatgctacgtatggcaaattgacgttgaaattcatttgtacgacg
ggcaaattgcctgttccttggcctacgttggttacgacgttcagctatgg
cgttcaatgtttcagccgttatcctgatcatatgaaacgtcatgatttct
tcaaaagcgctatgcctgaaggctatgttcaagaacgtacgattagcttc
aaagatgatggcaattataaaacgcgtgctgaagttaaattcgaaggcga
tacgttggttaatcgtattgaattgaaaggcattgatttcaaagaagatg
gcaatattttgggccataaattggaatataattataatagccataatgtt
tatattacggctgataaacaaaaaaatggcattaaagctaatttcaaaat
tcgtcataatattgaagatggcagcgttcaattggctgatcattatcaac
aaaatacgcctattggcgatggccctgttttgttgcctgataatcattat
ttgagcacgcaaagcgctttgagcaaagatcctaatgaaaaacgtgatca
tatggttttgttggaattcgttacggctgctggcattacgcatggcatgg
atgaattgtataaataataa

FIG. 43D shows a dot blot of NO-GFP constructs. E. coli Nissle harboring the nitric oxide inducible NsrR-GFP reporter fusion were grown overnight in LB supplemented with kanamycin. Bacteria were then diluted 1:100 into LB containing kanamycin and grown to an optical density of 0.4-0.5 and then pelleted by centrifugation. Bacteria were resuspended in phosphate buffered saline and 100 microliters were administered by oral gavage to mice. IBD is induced in mice by supplementing drinking water with 2-3% dextran sodium sulfate for 7 days prior to bacterial gavage. At 4 hours post-gavage, mice were sacrificed and bacteria were recovered from colonic samples. Colonic contents were boiled in SDS, and the soluble fractions were used to perform a dot blot for GFP detection (induction of NsrR-regulated promoters). Detection of GFP was performed by binding of anti-GFP antibody conjugated to HRP (horse radish peroxidase). Detection was visualized using Pierce chemiluminescent detection kit. It is shown in the figure that NsrR-regulated promoters are induced in DSS-treated mice, but are not shown to be induced in untreated mice. This is consistent with the role of NsrR in response to NO, and thus inflammation.

Bacteria harboring a plasmid expressing NsrR under control of a constitutive promoter and the reporter gene gfp (green fluorescent protein) under control of an NsrR-inducible promoter were grown overnight in LB supplemented with kanamycin. Bacteria are then diluted 1:100 into LB containing kanamycin and grown to an optical density of about 0.4-0.5 and then pelleted by centrifugation. Bacteria are resuspended in phosphate buffered saline and 100 microliters were administered by oral gavage to mice. IBD is induced in mice by supplementing drinking water with 2-3% dextran sodium sulfate for 7 days prior to bacterial gavage. At 4 hours post-gavage, mice were sacrificed and bacteria were recovered from colonic samples. Colonic contents were boiled in SDS, and the soluble fractions were used to perform a dot blot for GFP detection (induction of NsrR-regulated promoters) Detection of GFP was performed by binding of anti-GFP antibody conjugated to HRP (horse radish peroxidase). Detection was visualized using Pierce chemiluminescent detection kit. FIG. 43 shows NsrR-regulated promoters are induced in DSS-treated mice, but not in untreated mice.

### Example 27. FNR Promoter Activity

In order to measure the promoter activity of different FNR promoters, the lacZ gene, as well as transcriptional and translational elements, were synthesized (Gen9, Cambridge, Mass.) and cloned into vector pBR322. The lacZ gene was placed under the control of any of the exemplary FNR promoter sequences disclosed in Table 3 and/or Table 4. The nucleotide sequences of these constructs are shown in Tables 48-52 (SEQ ID NO: 65-69). However, as noted above, the lacZ gene may be driven by other inducible promoters in order to analyze activities of those promoters, and other genes may be used in place of the lacZ gene as a readout for promoter activity, exemplary results are shown in FIG. 41.

Table 48 shows the nucleotide sequence of an exemplary construct comprising a gene encoding lacZ, and an exemplary FNR promoter, Pfnr1 (SEQ ID NO: 65). The construct comprises a translational fusion of the Nissle nirB1 gene and the lacZ gene, in which the translational fusions are fused in frame to the 8th codon of the lacZ coding region. The Pfnr1 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 49 shows the nucleotide sequence of an exemplary construct comprising a gene encoding lacZ, and an exemplary FNR promoter, Pfnr2 (SEQ ID NO: 66). The construct comprises a translational fusion of the Nissle ydfZ gene and the lacZ gene, in which the translational fusions are fused in frame to the 8th codon of the lacZ coding region. The Pfnr2 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 50 shows the nucleotide sequence of an exemplary construct comprising a gene encoding lacZ, and an exemplary FNR promoter, Pfnr3 (SEQ ID NO: 67). The construct comprises a transcriptional fusion of the Nissle nirB gene and the lacZ gene, in which the transcriptional fusions use only the promoter region fused to a strong ribosomal binding site. The Pfnr3 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 51 shows the nucleotide sequence of an exemplary construct comprising a gene encoding lacZ, and an exemplary FNR promoter, Pfnr4 (SEQ ID NO: 68). The construct comprises a transcriptional fusion of the Nissle ydfZ gene and the lacZ gene. The Pfnr4 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 52 shows the nucleotide sequence of an exemplary construct comprising a gene encoding lacZ, and an exemplary FNR promoter, PfnrS (SEQ ID NO: 69). The construct comprises a transcriptional fusion of the anaerobically induced small RNA gene, fnrS1, fused to lacZ. The PfnrS sequence is bolded lower case, and the predicted ribosome binding site within the promoter is underlined. The lacZ sequence is underlined upper case. ATG site is bolded upper case, and the cloning sites used to synthesize the construct are shown in regular upper case.

TABLE 48
Pfnr1-lacZ Construct Sequences

Nucleotide sequences of Pfnr1-lacZ construct,
low-copy (SEQ ID NO: 65)
GGTACCgtcagcataacaccctgacctctcattaattgttcatgccgggc
ggcactatcgtcgtccggccttttcctctcttactctgctacgtacatct
atttctataaatccgttcaatttgtctgttttttgcacaaacatgaaata
tcagacaattccgtgacttaagaaaatttatacaaatcagcaatataccc
cttaaggagtatataaaggtgaatttgatttacatcaataagcggggttg
ctgaatcgttaaggtaggcggtaatagaaaagaaatcgaggcaaaaATGa
gcaaagtcagactcgcaattatGGATCCTCTGGCCGTCGTATTACAACGT
CGTGACTGGGAAAACCCTGGCGTTACCCAACTTAATCGCCTTGCGGCACA
TCCCCCTTTCGCCAGCTGGCGTAATAGCGAAGAGGCCCGCACCGATCGCC
CTTCCCAACAGTTGCGCAGCCTGAATGGCGAATGGCGCTTTGCCTGGTTT
CCGGCACCAGAAGCGGTGCCGGAAAGCTGGCTGGAGTGCGATCTTCCTGA
CGCCGATACTGTCGTCGTCCCCTCAAACTGGCAGATGCACGGTTACGATG
CGCCTATCTACACCAACGTGACCTATCCCATTACGGTCAATCCGCCGTTT
GTTCCCGCGGAGAATCCGACAGGTTGTTACTCGCTCACATTTAATATTGA
TGAAAGCTGGCTACAGGAAGGCCAGACGCGAATTATTTTTGATGGCGTTA
ACTCGGCGTTTCATCTGTGGTGCAACGGGCGCTGGGTCGGTTACGGCCAG
GACAGCCGTTTGCCGTCTGAATTTGACCTGAGCGCATTTTTACGCGCCGG
AGAAAACCGCCTCGCGGTGATGGTGCTGCGCTGGAGTGACGGCAGTTATC
TGGAAGATCAGGATATGTGGCGGATGAGCGGCATTTTCCGTGACGTCTCG
TTGCTGCATAAACCGACCACGCAAATCAGCGATTTCCAAGTTACCACTCT
CTTTAATGATGATTTCAGCCGCGCGGTACTGGAGGCAGAAGTTCAGATGT
ACGGCGAGCTGCGCGATGAACTGCGGGTGACGGTTTCTTTGTGGCAGGGT
GAAACGCAGGTCGCCAGCGGCACCGCGCCTTTCGGCGGTGAAATTATCGA
TGAGCGTGGCGGTTATGCCGATCGCGTCACACTACGCCTGAACGTTGAAA
ATCCGGAACTGTGGAGCGCCGAAATCCCGAATCTCTATCGTGCAGTGGTT
GAACTGCACACCGCCGACGGCACGCTGATTGAAGCAGAAGCCTGCGACGT
CGGTTTCCGCGAGGTGCGGATTGAAAATGGTCTGCTGCTGCTGAACGGCA
AGCCGTTGCTGATTCGCGGCGTTAACCGTCACGAGCATCATCCTCTGCAT
GGTCAGGTCATGGATGAGCAGACGATGGTGCAGGATATCCTGCTGATGAA
GCAGAACAACTTTAACGCCGTGCGCTGTTCGCATTATCCGAACCATCCGC
TGTGGTACACGCTGTGCGACCGCTACGGCCTGTATGTGGTGGATGAAGCC
AATATTGAAACCCACGGCATGGTGCCAATGAATCGTCTGACCGATGATCC
GCGCTGGCTACCCGCGATGAGCGAACGCGTAACGCGGATGGTGCAGCGCG
ATCGTAATCACCCGAGTGTGATCATCTGGTCGCTGGGGAATGAATCAGGC
CACGGCGCTAATCACGACGCGCTGTATCGCTGGATCAAATCTGTCGATCC
TTCCCGCCCGGTACAGTATGAAGGCGGCGGAGCCGACACCACGGCCACCG
ATATTATTTGCCCGATGTACGCGCGCGTGGATGAAGACCAGCCCTTCCCG
GCGGTGCCGAAATGGTCCATCAAAAAATGGCTTTCGCTGCCTGGAGAAAT
GCGCCCGCTGATCCTTTGCGAATATGCCCACGCGATGGGTAACAGTCTTG
GCGGCTTCGCTAAATACTGGCAGGCGTTTCGTCAGTACCCCCGTTTACAG
GGCGGCTTCGTCTGGGACTGGGTGGATCAGTCGCTGATTAAATATGATGA
AAACGGCAACCCGTGGTCGGCTTACGGCGGTGATTTTGGCGATACGCCGA
ACGATCGCCAGTTCTGTATGAACGGTCTGGTCTTTGCCGACCGCACGCCG
CATCCGGCGCTGACGGAAGCAAAACACCAACAGCAGTATTTCCAGTTCCG
TTTATCCGGGCGAACCATCGAAGTGACCAGCGAATACCTGTTCCGTCATA
GCGATAACGAGTTCCTGCACTGGATGGTGGCACTGGATGGCAAGCCGCTG
GCAAGCGGTGAAGTGCCTCTGGATGTTGGCCCGCAAGGTAAGCAGTTGAT
TGAACTGCCTGAACTGCCGCAGCCGGAGAGCGCCGGACAACTCTGGCTAA
CGGTACGCGTAGTGCAACCAAACGCGACCGCATGGTCAGAAGCCGGACAC
ATCAGCGCCTGGCAGCAATGGCGTCTGGCGGAAAACCTCAGCGTGACACT
CCCCTCCGCGTCCCACGCCATCCCTCAACTGACCACCAGCGGAACGGATT
TTTGCATCGAGCTGGGTAATAAGCGTTGGCAATTTAACCGCCAGTCAGGC
TTTCTTTCACAGATGTGGATTGGCGATGAAAAACAACTGCTGACCCCGCT
GCGCGATCAGTTCACCCGTGCGCCGCTGGATAACGACATTGGCGTAAGTG
AAGCGACCCGCATTGACCCTAACGCCTGGGTCGAACGCTGGAAGGCGGCG
GGCCATTACCAGGCCGAAGCGGCGTTGTTGCAGTGCACGGCAGATACACT
TGCCGACGCGGTGCTGATTACAACCGCCCACGCGTGGCAGCATCAGGGGA
AAACCTTATTTATCAGCCGGAAAACCTACCGGATTGATGGGCACGGTGAG
ATGGTCATCAATGTGGATGTTGCGGTGGCAAGCGATACACCGCATCCGGC
GCGGATTGGCCTGACCTGCCAGCTGGCGCAGGTCTCAGAGCGGGTAAACT
GGCTCGGCCTGGGGCCGCAAGAAAACTATCCCGACCGCCTTACTGCAGCC
TGTTTTGACCGCTGGGATCTGCCATTGTCAGACATGTATACCCCGTACGT
CTTCCCGAGCGAAAACGGTCTGCGCTGCGGGACGCGCGAATTGAATTATG
GCCCACACCAGTGGCGCGGCGACTTCCAGTTCAACATCAGCCGCTACAGC
CAACAACAACTGATGGAAACCAGCCATCGCCATCTGCTGCACGCGGAAGA
AGGCACATGGCTGAATATCGACGGTTTCCATATGGGGATTGGTGGCGACG
ACTCCTGGAGCCCGTCAGTATCGGCGGAATTCCAGCTGAGCGCCGGTCGC
TACCATTACCAGTTGGTCTGGTGTCAAAAATAA

TABLE 49
Pfnr2-lacZ Construct Sequences

Nucleotide sequences of Pfnr2-lacZ construct,
low-copy (SEQ ID NO: 66)
GGTACCcatttcctctcatcccatccggggtgagagtcttttcccccgac
ttatggctcatgcatgcatcaaaaaagatgtgagcttgatcaaaaacaaa
aaatatttcactcgacaggagtatttatattgcgcccgttacgtgggctt
cgactgtaaatcagaaaggagaaaacacctATGacgacctacgatcgGGA
TCCTCTGGCCGTCGTATTACAACGTCGTGACTGGGAAAACCCTGGCGTTA
CCCAACTTAATCGCCTTGCGGCACATCCCCCTTTCGCCAGCTGGCGTAAT
AGCGAAGAGGCCCGCACCGATCGCCCTTCCCAACAGTTGCGCAGCCTGAA
TGGCGAATGGCGCTTTGCCTGGTTTCCGGCACCAGAAGCGGTGCCGGAAA
GCTGGCTGGAGTGCGATCTTCCTGACGCCGATACTGTCGTCGTCCCCTCA
AACTGGCAGATGCACGGTTACGATGCGCCTATCTACACCAACGTGACCTA
TCCCATTACGGTCAATCCGCCGTTTGTTCCCGCGGAGAATCCGACAGGTT
GTTACTCGCTCACATTTAATATTGATGAAAGCTGGCTACAGGAAGGCCAG
ACGCGAATTATTTTTGATGGCGTTAACTCGGCGTTTCATCTGTGGTGCAA
CGGGCGCTGGGTCGGTTACGGCCAGGACAGCCGTTTGCCGTCTGAATTTG
ACCTGAGCGCATTTTTACGCGCCGGAGAAAACCGCCTCGCGGTGATGGTG
CTGCGCTGGAGTGACGGCAGTTATCTGGAAGATCAGGATATGTGGCGGAT
GAGCGGCATTTTCCGTGACGTCTCGTTGCTGCATAAACCGACCACGCAAA
TCAGCGATTTCCAAGTTACCACTCTCTTTAATGATGATTTCAGCCGCGCG
GTACTGGAGGCAGAAGTTCAGATGTACGGCGAGCTGCGCGATGAACTGCG
GGTGACGGTTTCTTTGTGGCAGGGTGAAACGCAGGTCGCCAGCGGCACCG
CGCCTTTCGGCGGTGAAATTATCGATGAGCGTGGCGGTTATGCCGATCGC
GTCACACTACGCCTGAACGTTGAAAATCCGGAACTGTGGAGCGCCGAAAT
CCCGAATCTCTATCGTGCAGTGGTTGAACTGCACACCGCCGACGGCACGC
TGATTGAAGCAGAAGCCTGCGACGTCGGTTTCCGCGAGGTGCGGATTGAA
AATGGTCTGCTGCTGCTGAACGGCAAGCCGTTGCTGATTCGCGGCGTTAA
CCGTCACGAGCATCATCCTCTGCATGGTCAGGTCATGGATGAGCAGACGA
TGGTGCAGGATATCCTGCTGATGAAGCAGAACAACTTTAACGCCGTGCGC
TGTTCGCATTATCCGAACCATCCGCTGTGGTACACGCTGTGCGACCGCTA
CGGCCTGTATGTGGTGGATGAAGCCAATATTGAAACCCACGGCATGGTGC
CAATGAATCGTCTGACCGATGATCCGCGCTGGCTACCCGCGATGAGCGAA
CGCGTAACGCGGATGGTGCAGCGCGATCGTAATCACCCGAGTGTGATCAT
CTGGTCGCTGGGGAATGAATCAGGCCACGGCGCTAATCACGACGCGCTGT
ATCGCTGGATCAAATCTGTCGATCCTTCCCGCCCGGTACAGTATGAAGGC
GGCGGAGCCGACACCACGGCCACCGATATTATTTGCCCGATGTACGCGCG
CGTGGATGAAGACCAGCCCTTCCCGGCGGTGCCGAAATGGTCCATCAAAA
AATGGCTTTCGCTGCCTGGAGAAATGCGCCCGCTGATCCTTTGCGAATAT
GCCCACGCGATGGGTAACAGTCTTGGCGGCTTCGCTAAATACTGGCAGGC
GTTTCGTCAGTACCCCCGTTTACAGGGCGGCTTCGTCTGGGACTGGGTGG
ATCAGTCGCTGATTAAATATGATGAAAACGGCAACCCGTGGTCGGCTTAC
GGCGGTGATTTTGGCGATACGCCGAACGATCGCCAGTTCTGTATGAACGG
TCTGGTCTTTGCCGACCGCACGCCGCATCCGGCGCTGACGGAAGCAAAAC
ACCAACAGCAGTATTTCCAGTTCCGTTTATCCGGGCGAACCATCGAAGTG
ACCAGCGAATACCTGTTCCGTCATAGCGATAACGAGTTCCTGCACTGGAT
GGTGGCACTGGATGGCAAGCCGCTGGCAAGCGGTGAAGTGCCTCTGGATG
TTGGCCCGCAAGGTAAGCAGTTGATTGAACTGCCTGAACTGCCGCAGCCG
GAGAGCGCCGGACAACTCTGGCTAACGGTACGCGTAGTGCAACCAAACGC
GACCGCATGGTCAGAAGCCGGACACATCAGCGCCTGGCAGCAATGGCGTC
TGGCGGAAAACCTCAGCGTGACACTCCCCTCCGCGTCCCACGCCATCCCT
CAACTGACCACCAGCGGAACGGATTTTTGCATCGAGCTGGGTAATAAGCG
TTGGCAATTTAACCGCCAGTCAGGCTTTCTTTCACAGATGTGGATTGGCG
ATGAAAAACAACTGCTGACCCCGCTGCGCGATCAGTTCACCCGTGCGCCG
CTGGATAACGACATTGGCGTAAGTGAAGCGACCCGCATTGACCCTAACGC
CTGGGTCGAACGCTGGAAGGCGGCGGGCCATTACCAGGCCGAAGCGGCGT
TGTTGCAGTGCACGGCAGATACACTTGCCGACGCGGTGCTGATTACAACC
GCCCACGCGTGGCAGCATCAGGGGAAAACCTTATTTATCAGCCGGAAAAC
CTACCGGATTGATGGGCACGGTGAGATGGTCATCAATGTGGATGTTGCGG
TGGCAAGCGATACACCGCATCCGGCGCGGATTGGCCTGACCTGCCAGCTG
GCGCAGGTCTCAGAGCGGGTAAACTGGCTCGGCCTGGGGCCGCAAGAAAA
CTATCCCGACCGCCTTACTGCAGCCTGTTTTGACCGCTGGGATCTGCCAT
TGTCAGACATGTATACCCCGTACGTCTTCCCGAGCGAAAACGGTCTGCGC
TGCGGGACGCGCGAATTGAATTATGGCCCACACCAGTGGCGCGGCGACTT
CCAGTTCAACATCAGCCGCTACAGCCAACAACAACTGATGGAAACCAGCC
ATCGCCATCTGCTGCACGCGGAAGAAGGCACATGGCTGAATATCGACGGT
TTCCATATGGGGATTGGTGGCGACGACTCCTGGAGCCCGTCAGTATCGGC
GGAATTCCAGCTGAGCGCCGGTCGCTACCATTACCAGTTGGTCTGGTGTC
AAAAATAA

TABLE 50
Pfnr3-lacZ Construct Sequences

Nucleotide sequences of Pfnr3-lacZ construct,
low-copy (SEQ ID NO: 67)
GGTACCgtcagcataacaccctgacctctcattaattgttcatgccgggc
ggcactatcgtcgtccggccttttcctctcttactctgctacgtacatct
atttctataaatccgttcaatttgtctgttttttgcacaaacatgaaata
tcagacaattccgtgacttaagaaaatttatacaaatcagcaatataccc
cttaaggagtatataaaggtgaatttgatttacatcaataagcggggttg
ctgaatcgttaaGGATCCctctagaaataattttgtttaactttaagaag
gagatatacatATGACTATGATTACGGATTCTCTGGCCGTCGTATTACAA
CGTCGTGACTGGGAAAACCCTGGCGTTACCCAACTTAATCGCCTTGCGGC
ACATCCCCCTTTCGCCAGCTGGCGTAATAGCGAAGAGGCCCGCACCGATC
GCCCTTCCCAACAGTTGCGCAGCCTGAATGGCGAATGGCGCTTTGCCTGG
TTTCCGGCACCAGAAGCGGTGCCGGAAAGCTGGCTGGAGTGCGATCTTCC
TGACGCCGATACTGTCGTCGTCCCCTCAAACTGGCAGATGCACGGTTACG
ATGCGCCTATCTACACCAACGTGACCTATCCCATTACGGTCAATCCGCCG
TTTGTTCCCGCGGAGAATCCGACAGGTTGTTACTCGCTCACATTTAATAT
TGATGAAAGCTGGCTACAGGAAGGCCAGACGCGAATTATTTTTGATGGCG
TTAACTCGGCGTTTCATCTGTGGTGCAACGGGCGCTGGGTCGGTTACGGC
CAGGACAGCCGTTTGCCGTCTGAATTTGACCTGAGCGCATTTTTACGCGC
CGGAGAAAACCGCCTCGCGGTGATGGTGCTGCGCTGGAGTGACGGCAGTT
ATCTGGAAGATCAGGATATGTGGCGGATGAGCGGCATTTTCCGTGACGTC
TCGTTGCTGCATAAACCGACCACGCAAATCAGCGATTTCCAAGTTACCAC
TCTCTTTAATGATGATTTCAGCCGCGCGGTACTGGAGGCAGAAGTTCAGA
TGTACGGCGAGCTGCGCGATGAACTGCGGGTGACGGTTTCTTTGTGGCAG
GGTGAAACGCAGGTCGCCAGCGGCACCGCGCCTTTCGGCGGTGAAATTAT
CGATGAGCGTGGCGGTTATGCCGATCGCGTCACACTACGCCTGAACGTTG
AAAATCCGGAACTGTGGAGCGCCGAAATCCCGAATCTCTATCGTGCAGTG
GTTGAACTGCACACCGCCGACGGCACGCTGATTGAAGCAGAAGCCTGCGA
CGTCGGTTTCCGCGAGGTGCGGATTGAAAATGGTCTGCTGCTGCTGAACG
GCAAGCCGTTGCTGATTCGCGGCGTTAACCGTCACGAGCATCATCCTCTG
CATGGTCAGGTCATGGATGAGCAGACGATGGTGCAGGATATCCTGCTGAT
GAAGCAGAACAACTTTAACGCCGTGCGCTGTTCGCATTATCCGAACCATC
CGCTGTGGTACACGCTGTGCGACCGCTACGGCCTGTATGTGGTGGATGAA
GCCAATATTGAAACCCACGGCATGGTGCCAATGAATCGTCTGACCGATGA
TCCGCGCTGGCTACCCGCGATGAGCGAACGCGTAACGCGGATGGTGCAGC
GCGATCGTAATCACCCGAGTGTGATCATCTGGTCGCTGGGGAATGAATCA
GGCCACGGCGCTAATCACGACGCGCTGTATCGCTGGATCAAATCTGTCGA
TCCTTCCCGCCCGGTACAGTATGAAGGCGGCGGAGCCGACACCACGGCCA
CCGATATTATTTGCCCGATGTACGCGCGCGTGGATGAAGACCAGCCCTTC
CCGGCGGTGCCGAAATGGTCCATCAAAAAATGGCTTTCGCTGCCTGGAGA
AATGCGCCCGCTGATCCTTTGCGAATATGCCCACGCGATGGGTAACAGTC
TTGGCGGCTTCGCTAAATACTGGCAGGCGTTTCGTCAGTACCCCCGTTTA
CAGGGCGGCTTCGTCTGGGACTGGGTGGATCAGTCGCTGATTAAATATGA
TGAAAACGGCAACCCGTGGTCGGCTTACGGCGGTGATTTTGGCGATACGC
CGAACGATCGCCAGTTCTGTATGAACGGTCTGGTCTTTGCCGACCGCACG
CCGCATCCGGCGCTGACGGAAGCAAAACACCAACAGCAGTATTTCCAGTT
CCGTTTATCCGGGCGAACCATCGAAGTGACCAGCGAATACCTGTTCCGTC
ATAGCGATAACGAGTTCCTGCACTGGATGGTGGCACTGGATGGCAAGCCG
CTGGCAAGCGGTGAAGTGCCTCTGGATGTTGGCCCGCAAGGTAAGCAGTT
GATTGAACTGCCTGAACTGCCGCAGCCGGAGAGCGCCGGACAACTCTGGC
TAACGGTACGCGTAGTGCAACCAAACGCGACCGCATGGTCAGAAGCCGGA
CACATCAGCGCCTGGCAGCAATGGCGTCTGGCGGAAAACCTCAGCGTGAC
ACTCCCCTCCGCGTCCCACGCCATCCCTCAACTGACCACCAGCGGAACGG
ATTTTTGCATCGAGCTGGGTAATAAGCGTTGGCAATTTAACCGCCAGTCA
GGCTTTCTTTCACAGATGTGGATTGGCGATGAAAAACAACTGCTGACCCC
GCTGCGCGATCAGTTCACCCGTGCGCCGCTGGATAACGACATTGGCGTAA
GTGAAGCGACCCGCATTGACCCTAACGCCTGGGTCGAACGCTGGAAGGCG
GCGGGCCATTACCAGGCCGAAGCGGCGTTGTTGCAGTGCACGGCAGATAC
ACTTGCCGACGCGGTGCTGATTACAACCGCCCACGCGTGGCAGCATCAGG
GGAAAACCTTATTTATCAGCCGGAAAACCTACCGGATTGATGGGCACGGT
GAGATGGTCATCAATGTGGATGTTGCGGTGGCAAGCGATACACCGCATCC
GGCGCGGATTGGCCTGACCTGCCAGCTGGCGCAGGTCTCAGAGCGGGTAA
ACTGGCTCGGCCTGGGGCCGCAAGAAAACTATCCCGACCGCCTTACTGCA
GCCTGTTTTGACCGCTGGGATCTGCCATTGTCAGACATGTATACCCCGTA
CGTCTTCCCGAGCGAAAACGGTCTGCGCTGCGGGACGCGCGAATTGAATT
ATGGCCCACACCAGTGGCGCGGCGACTTCCAGTTCAACATCAGCCGCTAC
AGCCAACAACAACTGATGGAAACCAGCCATCGCCATCTGCTGCACGCGGA
AGAAGGCACATGGCTGAATATCGACGGTTTCCATATGGGGATTGGTGGCG
ACGACTCCTGGAGCCCGTCAGTATCGGCGGAATTCCAGCTGAGCGCCGGT
CGCTACCATTACCAGTTGGTCTGGTGTCAAAAATAA

TABLE 51
Pfnr4-lacZ construct Sequences

Nucleotide sequences of Pfnr4-lacZ construct,
low-copy (SEQ ID NO: 68)
GGTACCcatttcctctcatcccatccggggtgagagtcttttcccccgac
ttatggctcatgcatgcatcaaaaaagatgtgagcttgatcaaaaacaaa
aaatatttcactcgacaggagtatttatattgcgcccGGATCCctctaga
aataattttgtttaactttaagaaggagatatacatATGACTATGATTAC
GGATTCTCTGGCCGTCGTATTACAACGTCGTGACTGGGAAAACCCTGGCG
TTACCCAACTTAATCGCCTTGCGGCACATCCCCCTTTCGCCAGCTGGCGT
AATAGCGAAGAGGCCCGCACCGATCGCCCTTCCCAACAGTTGCGCAGCCT
GAATGGCGAATGGCGCTTTGCCTGGTTTCCGGCACCAGAAGCGGTGCCGG
AAAGCTGGCTGGAGTGCGATCTTCCTGACGCCGATACTGTCGTCGTCCCC
TCAAACTGGCAGATGCACGGTTACGATGCGCCTATCTACACCAACGTGAC
CTATCCCATTACGGTCAATCCGCCGTTTGTTCCCGCGGAGAATCCGACAG
GTTGTTACTCGCTCACATTTAATATTGATGAAAGCTGGCTACAGGAAGGC
CAGACGCGAATTATTTTTGATGGCGTTAACTCGGCGTTTCATCTGTGGTG
CAACGGGCGCTGGGTCGGTTACGGCCAGGACAGCCGTTTGCCGTCTGAAT
TTGACCTGAGCGCATTTTTACGCGCCGGAGAAAACCGCCTCGCGGTGATG
GTGCTGCGCTGGAGTGACGGCAGTTATCTGGAAGATCAGGATATGTGGCG
GATGAGCGGCATTTTCCGTGACGTCTCGTTGCTGCATAAACCGACCACGC
AAATCAGCGATTTCCAAGTTACCACTCTCTTTAATGATGATTTCAGCCGC
GCGGTACTGGAGGCAGAAGTTCAGATGTACGGCGAGCTGCGCGATGAACT
GCGGGTGACGGTTTCTTTGTGGCAGGGTGAAACGCAGGTCGCCAGCGGCA
CCGCGCCTTTCGGCGGTGAAATTATCGATGAGCGTGGCGGTTATGCCGAT
CGCGTCACACTACGCCTGAACGTTGAAAATCCGGAACTGTGGAGCGCCGA
AATCCCGAATCTCTATCGTGCAGTGGTTGAACTGCACACCGCCGACGGCA
CGCTGATTGAAGCAGAAGCCTGCGACGTCGGTTTCCGCGAGGTGCGGATT
GAAAATGGTCTGCTGCTGCTGAACGGCAAGCCGTTGCTGATTCGCGGCGT
TAACCGTCACGAGCATCATCCTCTGCATGGTCAGGTCATGGATGAGCAGA
CGATGGTGCAGGATATCCTGCTGATGAAGCAGAACAACTTTAACGCCGTG
CGCTGTTCGCATTATCCGAACCATCCGCTGTGGTACACGCTGTGCGACCG
CTACGGCCTGTATGTGGTGGATGAAGCCAATATTGAAACCCACGGCATGG
TGCCAATGAATCGTCTGACCGATGATCCGCGCTGGCTACCCGCGATGAGC
GAACGCGTAACGCGGATGGTGCAGCGCGATCGTAATCACCCGAGTGTGAT
CATCTGGTCGCTGGGGAATGAATCAGGCCACGGCGCTAATCACGACGCGC
TGTATCGCTGGATCAAATCTGTCGATCCTTCCCGCCCGGTACAGTATGAA
GGCGGCGGAGCCGACACCACGGCCACCGATATTATTTGCCCGATGTACGC
GCGCGTGGATGAAGACCAGCCCTTCCCGGCGGTGCCGAAATGGTCCATCA
AAAAATGGCTTTCGCTGCCTGGAGAAATGCGCCCGCTGATCCTTTGCGAA
TATGCCCACGCGATGGGTAACAGTCTTGGCGGCTTCGCTAAATACTGGCA
GGCGTTTCGTCAGTACCCCCGTTTACAGGGCGGCTTCGTCTGGGACTGGG
TGGATCAGTCGCTGATTAAATATGATGAAAACGGCAACCCGTGGTCGGCT
TACGGCGGTGATTTTGGCGATACGCCGAACGATCGCCAGTTCTGTATGAA
CGGTCTGGTCTTTGCCGACCGCACGCCGCATCCGGCGCTGACGGAAGCAA
AACACCAACAGCAGTATTTCCAGTTCCGTTTATCCGGGCGAACCATCGAA
GTGACCAGCGAATACCTGTTCCGTCATAGCGATAACGAGTTCCTGCACTG
GATGGTGGCACTGGATGGCAAGCCGCTGGCAAGCGGTGAAGTGCCTCTGG
ATGTTGGCCCGCAAGGTAAGCAGTTGATTGAACTGCCTGAACTGCCGCAG
CCGGAGAGCGCCGGACAACTCTGGCTAACGGTACGCGTAGTGCAACCAAA
CGCGACCGCATGGTCAGAAGCCGGACACATCAGCGCCTGGCAGCAATGGC
GTCTGGCGGAAAACCTCAGCGTGACACTCCCCTCCGCGTCCCACGCCATC
CCTCAACTGACCACCAGCGGAACGGATTTTTGCATCGAGCTGGGTAATAA
GCGTTGGCAATTTAACCGCCAGTCAGGCTTTCTTTCACAGATGTGGATTG
GCGATGAAAAACAACTGCTGACCCCGCTGCGCGATCAGTTCACCCGTGCG
CCGCTGGATAACGACATTGGCGTAAGTGAAGCGACCCGCATTGACCCTAA
CGCCTGGGTCGAACGCTGGAAGGCGGCGGGCCATTACCAGGCCGAAGCGG
CGTTGTTGCAGTGCACGGCAGATACACTTGCCGACGCGGTGCTGATTACA
ACCGCCCACGCGTGGCAGCATCAGGGGAAAACCTTATTTATCAGCCGGAA
AACCTACCGGATTGATGGGCACGGTGAGATGGTCATCAATGTGGATGTTG
CGGTGGCAAGCGATACACCGCATCCGGCGCGGATTGGCCTGACCTGCCAG
CTGGCGCAGGTCTCAGAGCGGGTAAACTGGCTCGGCCTGGGGCCGCAAGA
AAACTATCCCGACCGCCTTACTGCAGCCTGTTTTGACCGCTGGGATCTGC
CATTGTCAGACATGTATACCCCGTACGTCTTCCCGAGCGAAAACGGTCTG
CGCTGCGGGACGCGCGAATTGAATTATGGCCCACACCAGTGGCGCGGCGA
CTTCCAGTTCAACATCAGCCGCTACAGCCAACAACAACTGATGGAAACCA
GCCATCGCCATCTGCTGCACGCGGAAGAAGGCACATGGCTGAATATCGAC
GGTTTCCATATGGGGATTGGTGGCGACGACTCCTGGAGCCCGTCAGTATC
GGCGGAATTCCAGCTGAGCGCCGGTCGCTACCATTACCAGTTGGTCTGGT
GTCAAAAATAA

TABLE 52
Pfnrs-lacZ construct Sequences

Nucleotide sequences of Pfnrs-lacZ construct,
low-copy (SEQ ID NO: 69)
GGTACCagttgttcttattggtggtgttgctttatggttgcatcgtagta
aatggttgtaacaaaagcaatttttccggctgtctgtatacaaaaacgcc
gtaaagtttgagcgaagtcaataaactctctacccattcagggcaatatc
tctcttGGATCCctctagaaataattttgtttaactttaagaaggagata
tacatATGCTATGATTACGGATTCTCTGGCCGTCGTATTACAACGTCGTG
ACTGGGAAAACCCTGGCGTTACCCAACTTAATCGCCTTGCGGCACATCCC
CCTTTCGCCAGCTGGCGTAATAGCGAAGAGGCCCGCACCGATCGCCCTTC
CCAACAGTTGCGCAGCCTGAATGGCGAATGGCGCTTTGCCTGGTTTCCGG
CACCAGAAGCGGTGCCGGAAAGCTGGCTGGAGTGCGATCTTCCTGACGCC
GATACTGTCGTCGTCCCCTCAAACTGGCAGATGCACGGTTACGATGCGCC
TATCTACACCAACGTGACCTATCCCATTACGGTCAATCCGCCGTTTGTTC
CCGCGGAGAATCCGACAGGTTGTTACTCGCTCACATTTAATATTGATGAA
AGCTGGCTACAGGAAGGCCAGACGCGAATTATTTTTGATGGCGTTAACTC
GGCGTTTCATCTGTGGTGCAACGGGCGCTGGGTCGGTTACGGCCAGGACA
GCCGTTTGCCGTCTGAATTTGACCTGAGCGCATTTTTACGCGCCGGAGAA
AACCGCCTCGCGGTGATGGTGCTGCGCTGGAGTGACGGCAGTTATCTGGA
AGATCAGGATATGTGGCGGATGAGCGGCATTTTCCGTGACGTCTCGTTGC
TGCATAAACCGACCACGCAAATCAGCGATTTCCAAGTTACCACTCTCTTT
AATGATGATTTCAGCCGCGCGGTACTGGAGGCAGAAGTTCAGATGTACGG
CGAGCTGCGCGATGAACTGCGGGTGACGGTTTCTTTGTGGCAGGGTGAAA
CGCAGGTCGCCAGCGGCACCGCGCCTTTCGGCGGTGAAATTATCGATGAG
CGTGGCGGTTATGCCGATCGCGTCACACTACGCCTGAACGTTGAAAATCC
GGAACTGTGGAGCGCCGAAATCCCGAATCTCTATCGTGCAGTGGTTGAAC
TGCACACCGCCGACGGCACGCTGATTGAAGCAGAAGCCTGCGACGTCGGT
TTCCGCGAGGTGCGGATTGAAAATGGTCTGCTGCTGCTGAACGGCAAGCC
GTTGCTGATTCGCGGCGTTAACCGTCACGAGCATCATCCTCTGCATGGTC
AGGTCATGGATGAGCAGACGATGGTGCAGGATATCCTGCTGATGAAGCAG
AACAACTTTAACGCCGTGCGCTGTTCGCATTATCCGAACCATCCGCTGTG
GTACACGCTGTGCGACCGCTACGGCCTGTATGTGGTGGATGAAGCCAATA
TTGAAACCCACGGCATGGTGCCAATGAATCGTCTGACCGATGATCCGCGC
TGGCTACCCGCGATGAGCGAACGCGTAACGCGGATGGTGCAGCGCGATCG
TAATCACCCGAGTGTGATCATCTGGTCGCTGGGGAATGAATCAGGCCACG
GCGCTAATCACGACGCGCTGTATCGCTGGATCAAATCTGTCGATCCTTCC
CGCCCGGTACAGTATGAAGGCGGCGGAGCCGACACCACGGCCACCGATAT
TATTTGCCCGATGTACGCGCGCGTGGATGAAGACCAGCCCTTCCCGGCGG
TGCCGAAATGGTCCATCAAAAAATGGCTTTCGCTGCCTGGAGAAATGCGC
CCGCTGATCCTTTGCGAATATGCCCACGCGATGGGTAACAGTCTTGGCGG
CTTCGCTAAATACTGGCAGGCGTTTCGTCAGTACCCCCGTTTACAGGGCG
GCTTCGTCTGGGACTGGGTGGATCAGTCGCTGATTAAATATGATGAAAAC
GGCAACCCGTGGTCGGCTTACGGCGGTGATTTTGGCGATACGCCGAACGA
TCGCCAGTTCTGTATGAACGGTCTGGTCTTTGCCGACCGCACGCCGCATC
CGGCGCTGACGGAAGCAAAACACCAACAGCAGTATTTCCAGTTCCGTTTA
TCCGGGCGAACCATCGAAGTGACCAGCGAATACCTGTTCCGTCATAGCGA
TAACGAGTTCCTGCACTGGATGGTGGCACTGGATGGCAAGCCGCTGGCAA
GCGGTGAAGTGCCTCTGGATGTTGGCCCGCAAGGTAAGCAGTTGATTGAA
CTGCCTGAACTGCCGCAGCCGGAGAGCGCCGGACAACTCTGGCTAACGGT
ACGCGTAGTGCAACCAAACGCGACCGCATGGTCAGAAGCCGGACACATCA
GCGCCTGGCAGCAATGGCGTCTGGCGGAAAACCTCAGCGTGACACTCCCC
TCCGCGTCCCACGCCATCCCTCAACTGACCACCAGCGGAACGGATTTTTG
CATCGAGCTGGGTAATAAGCGTTGGCAATTTAACCGCCAGTCAGGCTTTC
TTTCACAGATGTGGATTGGCGATGAAAAACAACTGCTGACCCCGCTGCGC
GATCAGTTCACCCGTGCGCCGCTGGATAACGACATTGGCGTAAGTGAAGC
GACCCGCATTGACCCTAACGCCTGGGTCGAACGCTGGAAGGCGGCGGGCC
ATTACCAGGCCGAAGCGGCGTTGTTGCAGTGCACGGCAGATACACTTGCC
GACGCGGTGCTGATTACAACCGCCCACGCGTGGCAGCATCAGGGGAAAAC
CTTATTTATCAGCCGGAAAACCTACCGGATTGATGGGCACGGTGAGATGG
TCATCAATGTGGATGTTGCGGTGGCAAGCGATACACCGCATCCGGCGCGG
ATTGGCCTGACCTGCCAGCTGGCGCAGGTCTCAGAGCGGGTAAACTGGCT
CGGCCTGGGGCCGCAAGAAAACTATCCCGACCGCCTTACTGCAGCCTGTT
TTGACCGCTGGGATCTGCCATTGTCAGACATGTATACCCCGTACGTCTTC
CCGAGCGAAAACGGTCTGCGCTGCGGGACGCGCGAATTGAATTATGGCCC
ACACCAGTGGCGCGGCGACTTCCAGTTCAACATCAGCCGCTACAGCCAAC
AACAACTGATGGAAACCAGCCATCGCCATCTGCTGCACGCGGAAGAAGGC
ACATGGCTGAATATCGACGGTTTCCATATGGGGATTGGTGGCGACGACTC
CTGGAGCCCGTCAGTATCGGCGGAATTCCAGCTGAGCGCCGGTCGCTACC
ATTACCAGTTGGTCTGGTGTCAAAAATAA

### Example 28. Sequences

In some embodiments, the genetically engineered bacteria comprise a gene cassette which is driven by a propionate responsive promoter. In a non-limiting example, the gene cassette is driven by the prpR Propionate-Responsive promoter. In a non-limiting example, the prpR Propionate-Responsive promoter has the sequence shown in Table 53.

TABLE 53
prpR Propionate-Responsive Promoter Sequence

| Description | Sequence | SEQ ID NO |
|---|---|---|
| Prp promoter - | 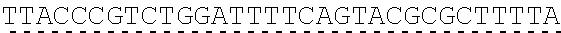 | SEQ ID NO: 70 |
| Highlight: prpR, | 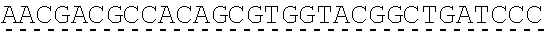 | |
| lower case:Ribosome | 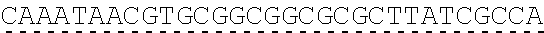 | |
| binding site, | 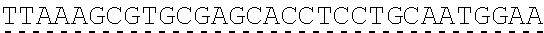 | |
| underlined atg: start | 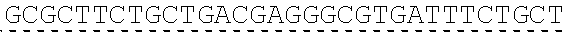 | |
| of gene of interest | 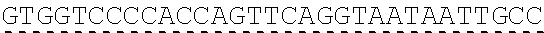 | |
| | 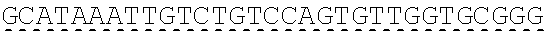 | |
| | 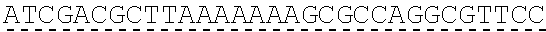 | |
| | 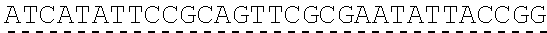 | |
| | 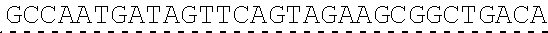 | |
| | 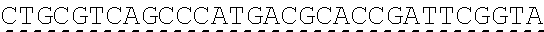 | |
| | 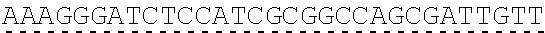 | |
| | 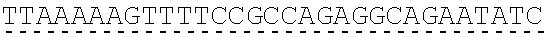 | |
| | 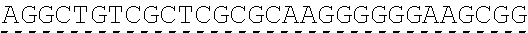 | |
| | 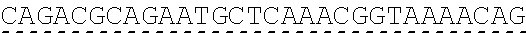 | |
| | 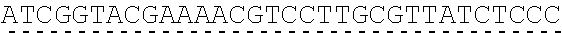 | |
| | 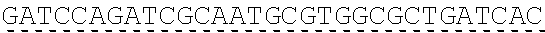 | |
| | 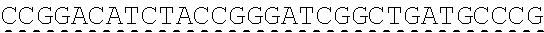 | |
| | 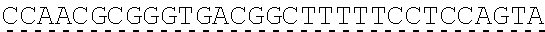 | |
| | 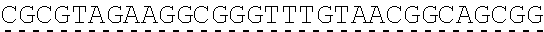 | |
| | 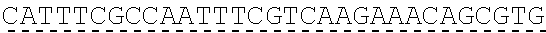 | |
| | 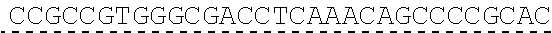 | |
| | 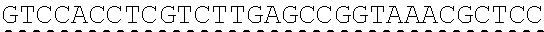 | |
| | 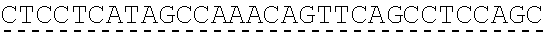 | |
| | 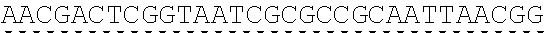 | |
| | 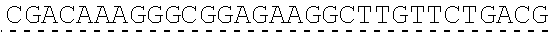 | |
| | 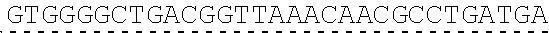 | |
| | 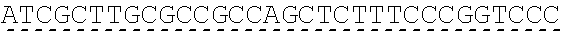 | |
| | 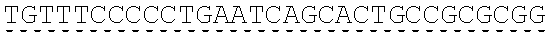 | |
| | 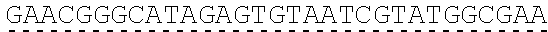 | |
| | 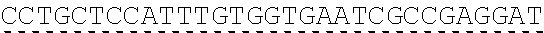 | |
| | 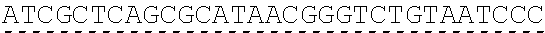 | |
| | 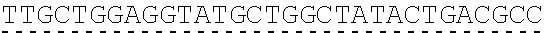 | |
| | 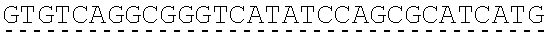 | |
| | 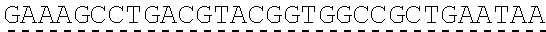 | |
| | 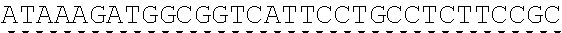 | |
| | 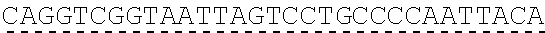 | |
| | 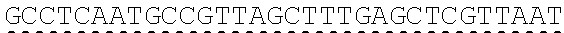 | |
| | 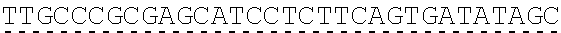 | |
| | 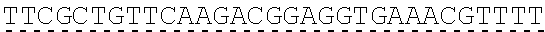 | |
| | 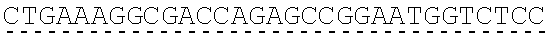 | |
| | 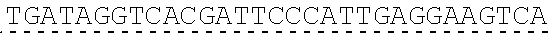 | |
| | 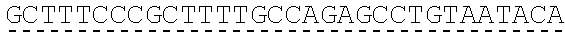 | |
| | 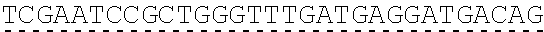 | |
| | 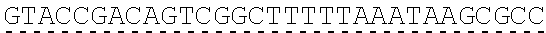 | |
| | 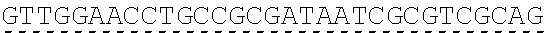 | |
| | 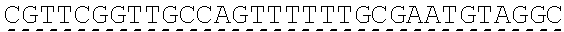 | |
| | 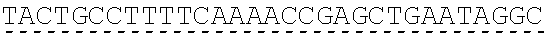 | |
| | 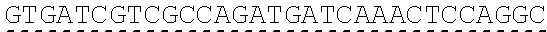 | |
| | 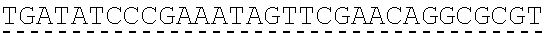 | |
| | 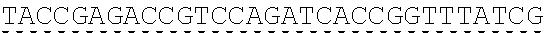 | |
| | 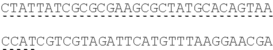 | |
| | ATTCTTGTTTTATAGATGTTTCGTTAATGTTG | |
| | CAATGAAACACAGGCCTCCGTTTCATGAAAC | |
| | GTTAGCTGACTCGTTTTTCTTGTGACTCGTCT | |
| | GTCAGTATTAAAAAAGATTTTTCATTTAACTG | |
| | ATTGTTTTTAAATTGAATTTTATTTAATGGTT | |
| | TCTCGGTTTTTGGGTCTGGCATATCCCTTGCT | |
| | TTAATGAGTGCATCTTAATTAACAATTCAATA | |
| | ACAAGAGGGCTGAATagtaatttcaacaaaat | |
| | aacgagcattcgaatg | |

TABLE 54
List of Sequences

| SEQ | Gene or | | |
|---|---|---|---|
| ID | Gene | | |
| NO: | Cassette | Origin | Sequence |
| 71 | PrpE | E. coli | MSFSEFYQRSINEPEKFWAEQARRIDWQTPFTQTLDHSNPPFA |
| | (polypeptide) | | RWFCEGRTNLCHNAIDRWLEKQPEALALIAVSSETEEERTFT |
| | | | FRQLHDEVNAVASMLRSLGVQRGDRVLVYMPMIAEAHITLL |
| | | | ACARIGAIHSVVFGGFASHSVATRIDDAKPVLIVSADAGARG |
| | | | GKIIPYKKLLDDAISQAQHQPRHVLLVDRGLAKMARVSGRD |
| | | | VDFASLRHQHIGARVPVAWLESNETSCILYTSGTTGKPKGVQ |
| | | | RDVGGYAVALATSMDTIFGGKAGGVFFCASDIGWVVGHSYI |
| | | | VYAPLLAGMATIVYEGLPTWPDCGVWWKIVEKYQVSRMFS |
| | | | APTAIRVLKKFPTAEIRKHDLSSLEVLYLAGEPLDEPTASWVS |
| | | | NTLDVPVIDNYWQTESGWPIMAIARGLDDRPTRLGSPGVPM |
| | | | YGYNVQLLNEVTGEPCGVNEKGMLVVEGPLPPGCIQTIWGD |
| | | | DDRFVKTYWSLFSRPVYATFDWGIRDADGYHFILGRTDDVIN |
| | | | VAGHRLGTREIEESISSHPGVAEVAVVGVKDALKGQVAVAF |
| | | | VIPKESDSLEDREVAHSQEKAIMALVDSQIGNFGRPAHVWFV |
| | | | SQLPKTRSGKMLRRTIQAICEGRDPGDLTTIDDPASLDQIRQA |
| | | | MEE |
| 72 | PrpE | Salmonella | MSFSEFYQRSINEPEQFWAEQARRIDWQQPFTQTLDYSNPPF |
| | (polypeptide) | | ARWFCGGTTNLCHNAIDRWLDTQPDALALIAVSSETEEERTF |
| | | | TFRQLYDEVNVVASMLLSLGVRRGDRVLVYMPMIAEAHITL |
| | | | LACARIGAIHSVVFGGFASHSVAARIDDARPVLIVSADAGAR |
| | | | GGKVIPYKKLLDEAVDQAQHQPKHVLLVDRGLAKMARVAG |
| | | | RDVDFATLREHHAGARVPVAWLESNESSCILYTSGTTGKPKG |
| | | | VQRDVGGYAVALATSMDTLFGGKAGGVFFCASDIGWVVGH |
| | | | SYIVYAPLLAGMATIVYEGLPTYPDCGVWWKIVEKYRVSRM |
| | | | FSAPTAIRVLKKFPTAQIRNHDLSSLEVLYLAGEPLDEPTAAW |
| | | | VSGTLGVPVIDNYWQTESGWPIMALARTLDDRPSRLGSPGVP |
| | | | MYGYNVQLLNEVTGEPCGANEKGMVVIEGPLPPGCIQTIWG |
| | | | DDARFVNTYWSLFTRQVYATFDWGIRDADGYYFILGRTDDV |
| | | | INVAGHRLGTREIEESISSYPNVAEVAVVGVKDALKGQVAVA |
| | | | FVIPKQSDSLEDREVAHSEEKAIMALVDSQIGNFGRPAHVWF |
| | | | VSQLPKTRSGKMLRRTIQAICEGRDPGDLTTIDDPTSLQQIRQ |
| | | | VIEE |
| 73 | prpE | Salmonella | ATGTCTTTTAGCGAATTTTATCAGCGTTCGATTAACGAACC |
| | | | GGAGCAGTTCTGGGCTGAACAGGCCCGGCGTATCGACTGG |
| | | | CAGCAGCCGTTTACGCAGACGCTGGACTACAGCAACCCGC |
| | | | CGTTTGCCCGCTGGTTTTGCGGCGGCACCACTAATCTGTGC |
| | | | CATAACGCGATTGACCGCTGGCTGGATACCCAGCCGGATG |
| | | | CGCTGGCGCTGATTGCGGTTTCCTCTGAGACCGAAGAAGA |
| | | | ACGTACCTTCACCTTTCGTCAACTGTATGACGAGGTGAAT |
| | | | GTCGTGGCCTCTATGCTGCTGTCACTGGGCGTGCGGCGTG |
| | | | GCGATCGGGTACTGGTGTATATGCCGATGATTGCCGAGGC |
| | | | GCACATCACATTACTGGCCTGCGCGCGCATTGGCGCGATC |
| | | | CATTCAGTGGTGTTTGGTGGTTTTGCCTCGCACAGTGTAGC |
| | | | CGCGCGCATCGACGATGCCAGACCGGTGCTGATTGTCTCG |
| | | | GCGGACGCCGGAGCGCGAGGTGGGAAGGTCATTCCCTATA |
| | | | AAAAGCTTCTTGATGAGGCGGTCGATCAGGCACAGCATCA |
| | | | GCCGAAGCATGTACTGCTGGTGGATCGGGGGCTGGCGAAA |
| | | | ATGGCGCGGGTTGCCGGGCGCGATGTGGATTTTGCGACCC |
| | | | TGCGCGAACACCATGCCGGGGCGCGTGTGCCAGTGGCCTG |
| | | | GCTTGAATCTAATGAAAGTTCCTGCATTCTTTATACCTCCG |
| | | | GCACTACCGGCAAACCGAAAGGCGTTCAGCGTGACGTTGG |
| | | | TGGCTACGCCGTGGCGCTGGCGACATCGATGGACACCCTC |
| | | | TTTGGCGGCAAAGCGGGCGGCGTCTTTTTCTGCGCTTCGG |
| | | | ATATCGGTTGGGTAGTGGGGCACTCTTATATTGTGTATGCG |
| | | | CCGCTGCTGGCGGGTATGGCGACCATCGTTTATGAAGGAT |
| | | | TGCCGACGTATCCGGACTGCGGCGTATGGTGGAAAATTGT |
| | | | CGAGAAATATCGGGTGAGCCGGATGTTTTCAGCGCCAACC |
| | | | GCCATTCGTGTGCTGAAGAAATTTCCCACCGCGCAGATAC |
| | | | GCAATCATGATCTCTCCTCGCTGGAAGTTCTCTATCTGGCA |
| | | | GGCGAGCCGCTCGACGAGCCAACGGCAGCCTGGGTTAGC |
| | | | GGAACACTGGGTGTGCCGGTGATCGACAATTACTGGCAGA |
| | | | CCGAATCCGGCTGGCCGATTATGGCGCTGGCGCGCACGCT |
| | | | TGATGACAGACCATCGCGTTTGGGCAGTCCCGGCGTGCCG |
| | | | ATGTACGGCTATAATGTTCAACTGCTCAACGAGGTGACCG |
| | | | GTGAACCCTGTGGTGCGAACGAAAAGGGAATGGTGGTTAT |
| | | | TGAAGGGCCGCTGCCGCCGGGCTGCATTCAGACCATCTGG |
| | | | GGCGATGACGCACGCTTTGTGAATACCTACTGGTCACTGT |
| | | | TTACTCGTCAGGTGTATGCCACCTTTGACTGGGGGATCCGC |
| | | | GACGCCGACGGCTATTATTTTATCCTTGGGCGCACGGATG |
| | | | ATGTGATCAACGTCGCCGGACATCGTCTCGGCACCCGTGA |
| | | | GATAGAGGAGAGCATCTCCAGCTATCCCAACGTTGCGGAA |
| | | | GTGGCGGTGGTAGGGGTAAAAGACGCGCTGAAAGGGCAG |
| | | | GTAGCGGTAGCCTTCGTGATCCCGAAACAGAGTGACAGTC |
| | | | TGGAAGACCGCGAAGTGGCGCATTCGGAAGAGAAGGCGA |
| | | | TTATGGCGCTGGTCGATAGTCAGATCGGCAACTTTGGCCG |
| | | | CCCGGCGCACGTGTGGTTTGTCTCGCAGCTACCAAAAACC |
| | | | CGATCCGGGAAGATGCTCAGACGAACGATCCAGGCGATCT |
| | | | GCGAGGGCCGGGATCCAGGCGATCTGACGACCATTGACG |
| | | | ATCCGACGTCGTTGCAACAAATTCGCCAGGTCATTGAGGA |
| | | | GTAA |
| 74 | PrpC | E. coli | MSDTTILQNSTHVIKPKKSVALSGVPAGNTALCTVGKSGNDL |
| | (polypeptide) | | HYRGYDILDLAEHCEFEEVAHLLIHGKLPTRDELAAYKTKLK |
| | | | ALRGLPANVRTVLEALPAASHPMDVMRTGVSALGCTLPEKE |
| | | | GHTVSGARDIADKLLASLSSILLYWYHYSHNGERIQPETDDD |
| | | | SIGGHFLHLLHGEKPSQSWEKAMHISLVLYAEHEFNASTFTS |
| | | | RVIAGTGSDMYSAIIGAIGALRGPKHGGANEVSLEIQQRYETP |
| | | | GEAEADIRKRVENKEVVIGFGHPVYTIADPRHQVIKRVAKQL |
| | | | SQEGGSLKMYNIADRLETVMWESKKMFPNLDWFSAVSYNM |
| | | | MGVPTEMFTPLFVIARVTGWAAHIIEQRQDNKIIRPSANYVGP |
| | | | EDRQFVALDKRQ |
| 75 | PrpC | Salmonella | MSDTTILQNNTNVIKPKKSVALSGVPAGNTALCTVGKSGNDL |
| | (polypeptide) | | HYRGYDILDLAEHCEFEEVAHLLIHGKLPTRDELNAYKSKLK |
| | | | ALRGLPANVRTVLEALPAASHPMDVMRTGVSALGCTLPEKE |
| | | | GHTVSGARDIADKLLASLSSILLYWYHYSHNGERIQPETDDD |
| | | | SIGGHFLHLLHGEKPSQSWEKAMHISLVLYAEHEFNASTFTS |
| | | | RVVAGTGSDMYSAIIGAIGALRGPKHGGANEVSLEIQQRYET |
| | | | PDEAEADIRKRIANKEVVIGFGHPVYTIADPRHQVIKRVAKQL |
| | | | SQEGGSLKMYNIADRLETVMWDSKKMFPNLDWFSAVSYNM |
| | | | MGVPTEMFTPLFVIARVTGWAAHIIEQRQDNKIIRPSANYIGP |
| | | | EDRAFTPLEQRQ |
| 76 | prpC | Salmonella | ATGAGCGACACGACGATCCTGCAAAACAACACAAATGTC |
| | | | ATTAAGCCAAAAAAATCCGTCGCATTATCCGGCGTACCCG |
| | | | CCGGAAATACCGCCTTATGCACCGTAGGTAAAAGCGGTAA |
| | | | CGATCTGCACTATCGCGGGTACGATATTCTCGATCTCGCG |
| | | | GAGCACTGTGAATTTGAAGAAGTTGCGCATCTGCTCATTC |
| | | | ACGGCAAGCTGCCCACCCGTGATGAGCTGAATGCCTATAA |
| | | | AAGCAAATTAAAAGCGCTGCGTGGCTTACCCGCTAACGTC |
| | | | CGTACCGTGCTGGAAGCGCTGCCAGCGGCATCGCACCCGA |
| | | | TGGACGTAATGCGCACCGGCGTTTCTGCGCTGGGCTGCAC |
| | | | CCTGCCGGAAAAAGAGGGGCATACCGTTTCTGGCGCGCGT |
| | | | GATATCGCCGACAAGCTGCTGGCCTCCCTCAGCTCCATTCT |
| | | | CCTTTACTGGTATCACTACAGCCACAACGGCGAACGCATT |
| | | | CAGCCAGAAACTGACGATGACTCTATCGGCGGGCATTTCC |
| | | | TGCATTTATTACACGGCGAAAAGCCATCGCAAAGCTGGGA |
| | | | AAAGGCGATGCACATTTCACTGGTACTGTACGCCGAACAT |
| | | | GAGTTCAACGCCTCAACCTTTACCAGCCGGGTGGTAGCCG |
| | | | GTACGGGATCGGATATGTACTCCGCCATCATTGGCGCGAT |
| | | | AGGCGCGCTTCGCGGGCCGAAGCACGGCGGGGCGAATGA |
| | | | AGTCTCGCTGGAGATTCAGCAGCGCTACGAAACGCCGGAT |
| | | | GAAGCAGAAGCCGATATCCGTAAACGTATCGCCAATAAA |
| | | | GAAGTGGTGATTGGTTTTGGTCATCCGGTATACACCATCG |
| | | | CCGATCCGCGCCATCAGGTGATTAAGCGGGTAGCGAAGCA |
| | | | GCTTTCACAGGAGGGCGGTTCGCTGAAGATGTACAACATT |
| | | | GCCGATCGGCTGGAGACGGTAATGTGGGACAGCAAAAAG |
| | | | ATGTTCCCTAATCTCGACTGGTTCTCGGCGGTCTCCTACAA |
| | | | CATGATGGGCGTTCCCACCGAAATGTTTACCCCGCTGTTTG |
| | | | TGATTGCCCGCGTTACAGGTTGGGCGGCGCACATCATCGA |
| | | | GCAACGACAGGACAACAAAATTATCCGTCCTTCCGCCAAT |
| | | | TATATTGGCCCGGAAGATCGCGCCTTTACGCCGCTGGAAC |
| | | | AGCGTCAGTAA |
| 77 | PrpD | E. coli | MSAQINNIRPEFDREIVDIVDYVMNYEISSRVAYDTAHYCLL |
| | (polypeptide) | | DTLGCGLEALEYPACKKLLGPIVPGTVVPNGVRVPGTQFQLD |
| | | | PVQAAFNIGAMIRWLDFNDTWLAAEWGHPSDNLGGILATAD |
| | | | WLSRNAIASGKAPLTMKQVLTGMIKAHEIQGCIALENSFNRV |
| | | | GLDHVLLVKVASTAVVAEMLGLTREEILNAVSLAWVDGQSL |
| | | | RTYRHAPNTGTRKSWAAGDATSRAVRLALMAKTGEMGYPS |
| | | | ALTAPVWGFYDVSFKGESFRFQRPYGSYVMENVLFKISFPAE |
| | | | FHSQTAVEAAMTLYEQMQAAGKTAADIEKVTIRTHEACIRII |
| | | | DKKGPLNNPADRDHCIQYMVAIPLLFGRLTAADYEDNVAQD |
| | | | KRIDALREKINCFEDPAFTADYHDPEKRAIANAITLEFTDGTR |
| | | | FEEVVVEYPIGHARRRQDGIPKLVDKFKINLARQFPTRQQQRI |
| | | | LEVSLDRTRLEQMPVNEYLDLYVI |
| 78 | PrpD | Salmonella | MSAPVSNVRPEFDREIVDIVDYVMKYNITSKVAYDTAHYCLL |
| | (polypeptide) | | DTLGCGLEALEYPACKKLMGPIVPGTVVPNGVRVPGTQFQL |
| | | | DPVQAAFNIGAMIRWLDFNDTWLAAEWGHPSDNLGGILATA |
| | | | DWLSRNAVAAGKAPLTMQQVLTGMIKAHEIQGCIALENSFN |
| | | | RVGLDHVLLVKVASTAVVAEMLGLTRDEILNAVSLAWVDG |
| | | | QSLRTYRHAPNTGTRKSWAAGDATSRAVRLALMAKTGEMG |
| | | | YPSALTAKTWGFYDVSFKGEKFRFQRPYGSYVMENVLFKISF |
| | | | PAEFHSQTAVEAAMTLYEQMQAAGKTAADIEKVTIRTHEACI |
| | | | RIIDKKGPLNNPADRDHCIQYMVAIPLLFGRLTAADYEDGVA |
| | | | QDKRIDALREKTHCFEDPAFTTDYHDPEKRSIANAISLEFTDG |
| | | | TRFDEVVVEYPIGHARRRGDGIPKLIEKFKINLARQFPPRQQQ |
| | | | RILDVSLDRTRLEQMPVNEYLDLYVI |
| 79 | prpD | Salmonella | ATGTCCGCACCTGTTTCGAACGTCCGCCCTGAATTTGACCG |
| | | | TGAAATTGTTGATATTGTTGATTATGTGATGAAGTACAACA |
| | | | TCACCTCAAAAGTGGCTTATGACACCGCGCACTACTGTCT |
| | | | GCTTGATACCCTGGGCTGTGGGCTGGAAGCGCTGGAATAT |
| | | | CCGGCCTGTAAAAAATTGATGGGGCCTATCGTGCCAGGTA |
| | | | CCGTGGTGCCGAACGGTGTACGTGTACCGGGCACTCAGTT |
| | | | CCAGCTCGATCCGGTGCAGGCGGCATTTAATATTGGCGCG |
| | | | ATGATCCGCTGGCTCGACTTTAACGATACCTGGCTTGCCGC |
| | | | TGAGTGGGGACACCCTTCCGATAACCTCGGCGGTATTCTG |
| | | | GCGACCGCCGACTGGTTGTCGCGCAACGCCGTCGCCGCCG |
| | | | GTAAAGCGCCGCTGACCATGCAGCAGGTGCTGACCGGGAT |
| | | | GATCAAAGCCCACGAAATCCAGGGCTGTATCGCGCTGGAA |
| | | | AACTCGTTTAACCGCGTGGGTCTCGATCACGTTTTGCTGGT |
| | | | GAAAGTGGCTTCCACGGCTGTAGTGGCTGAAATGCTCGGC |
| | | | CTGACCCGCGATGAAATTCTCAACGCCGTATCGCTGGCGT |
| | | | GGGTGGATGGGCAGTCGCTGCGTACCTATCGCCATGCGCC |
| | | | AAACACCGGTACGCGCAAATCCTGGGCGGCAGGCGATGC |
| | | | CACTTCACGCGCGGTGCGTCTGGCGCTGATGGCGAAAACT |
| | | | GGCGAGATGGGCTATCCCTCGGCGTTGACCGCCAAAACCT |
| | | | GGGGCTTTTATGACGTCTCGTTCAAAGGCGAAAAATTCCG |
| | | | TTTCCAGCGCCCGTACGGCTCCTACGTGATGGAAAACGTG |
| | | | CTGTTCAAAATCTCCTTCCCGGCGGAGTTCCATTCGCAGAC |
| | | | CGCCGTTGAAGCAGCGATGACGCTGTATGAGCAGATGCAG |
| | | | GCGGCTGGAAAAACGGCGGCGGATATCGAAAAAGTAACG |
| | | | ATTCGCACCCATGAAGCCTGTATACGCATCATTGATAAAA |
| | | | AAGGCCCGCTGAATAATCCGGCTGACCGCGATCACTGTAT |
| | | | TCAGTATATGGTGGCGATCCCACTGCTGTTCGGACGCTTA |
| | | | ACGGCGGCGGATTATGAGGATGGCGTGGCGCAGGATAAA |
| | | | CGTATTGACGCGCTGCGTGAAAAAACGCATTGCTTTGAAG |
| | | | ACCCGGCGTTTACCACTGATTATCATGACCCGGAAAAACG |
| | | | TTCGATTGCCAACGCCATTAGTCTTGAATTTACTGACGGTA |
| | | | CCCGTTTTGACGAGGTGGTTGTCGAGTACCCGATCGGCCA |
| | | | CGCGCGTCGTCGCGGCGACGGCATTCCAAAACTTATCGAA |
| | | | AAATTTAAAATCAATCTGGCGCGCCAGTTCCCACCCCGCC |
| | | | AGCAACAACGCATCCTGGATGTCTCCCTGGACAGAACGCG |
| | | | CCTGGAGCAGATGCCGGTTAATGAGTATCTCGACTTGTAC |
| | | | GTCATCTAG |
| 80 | PrpB | E. coli | MSLHSPGKAFRAALSKETPLQIVGTINANHALLAQRAGYQAI |
| | (polypeptide) | | YLSGGGVAAGSLGLPDLGISTLDDVLTDIRRITDVCSLPLLVD |
| | | | ADIGFGSSAFNVARTVKSMIKAGAAGLHIEDQVGAKRCGHR |
| | | | PNKAIVSKEEMVDRIRAAVDAKTDPDFVIMARTDALAVEGL |
| | | | DAAIERAQAYVEAGAEMLFPEAITELAMYRQFADAVQVPILS |
| | | | NITEFGATPLFTTDELRSAHVAMALYPLSAFRAMNRAAEHV |
| | | | YNILRQEGTQKSVIDTMQTRNELYESINYYQYEEKLDDLFAR |
| | | | GQVK |
| 81 | PrpB | Salmonella | MTLHSPGQAFRAALAKEKPLQIVGAINANHALLAQRAGYQA |
| | (polypeptide) | | LYLSGGGVAAGSLGLPDLGISTLDDVLTDIRRITDVCPLPLLV |
| | | | DADIGFGSSAFNVARTVKSISKAGAAALHIEDQIGAKRCGHR |
| | | | PNKAIVSKEEMVDRIHAAVDARTDPDFVIMARTDALAVEGL |
| | | | DAAIDRARAYVEAGADMLFPEAITELAMYRQFADAVQVPIL |
| | | | ANITEFGATPLFTTEELRNANVAMALYPLSAFRAMNRAAEK |
| | | | VYNVLRQEGTQKSVIDIMQTRNELYESINYYQFEEKLDALYA |
| | | | KKS |
| 82 | prpB | Salmonella | ATGACGTTACACTCACCGGGTCAGGCGTTTCGCGCTGCGC |
| | | | TTGCTAAAGAAAAACCATTACAAATTGTCGGCGCTATCAA |
| | | | CGCCAATCATGCTCTGTTAGCCCAGAGGGCTGGGTATCAG |
| | | | GCTCTCTATCTCTCGGGCGGCGGTGTTGCCGCAGGCTCGCT |
| | | | GGGGCTACCGGATCTGGGCATCTCCACCCTTGATGACGTA |
| | | | TTGACCGATATCCGCCGTATCACCGACGTCTGCCCGCTGCC |
| | | | GCTGCTGGTGGATGCCGATATTGGCTTCGGATCGTCGGCG |
| | | | TTTAACGTAGCGCGTACCGTGAAATCGATTTCCAAAGCCG |
| | | | GCGCCGCCGCGCTGCATATTGAAGATCAGATTGGCGCCAA |
| | | | GCGCTGCGGGCATCGGCCAAATAAAGCGATCGTCTCGAAA |
| | | | GAAGAGATGGTGGACCGGATCCACGCGGCGGTGGATGCG |
| | | | CGGACCGATCCTGACTTTGTCATTATGGCGCGTACCGATG |
| | | | CGCTGGCGGTTGAAGGCCTTGATGCCGCTATCGATCGCGC |
| | | | GCGGGCCTACGTAGAGGCCGGTGCCGACATGCTGTTCCCG |
| | | | GAGGCGATTACTGAACTTGCGATGTACCGCCAGTTTGCCG |
| | | | ACGCAGTGCAGGTGCCAATCCTTGCCAATATTACCGAATT |
| | | | CGGCGCGACGCCGTTGTTTACTACCGAAGAGCTACGCAAC |
| | | | GCCAACGTGGCGATGGCGCTCTATCCGCTGTCGGCGTTCC |
| | | | GGGCGATGAATCGCGCGGCGGAGAAGGTTTACAACGTGCT |
| | | | GCGACAGGAAGGAACGCAAAAGAGCGTTATCGACATCAT |
| | | | GCAGACCCGTAATGAGCTGTATGAAAGCATCAATTATTAC |
| | | | CAGTTCGAGGAAAAACTTGACGCGCTGTACGCCAAAAAAT |
| | | | CGTAG |
| 83 | prpBCD | E. coli | atgtctctacactctccaggtaaagcgtttcgcgctgcacttagcaaagaaaccccgttgcaaattg |
| | | | ttggcaccatcaacgctaaccatgcgctgctggcgcagcgtgccggatatcaggcgatttatctct |
| | | | ccggcggtggcgtggcggcaggatcgctggggctgcccgatctcggtatttctactcttgatgac |
| | | | gtgctgacagatattcgccgtatcaccgacgtttgttcgctgccgctgctggtggatgcggatatc |
| | | | ggttttggttcttcagcctttaacgtggcgcgtacggtgaaatcaatgattaaagccggtgcggca |
| | | | ggattgcatattgaagatcaggttggtgcgaaacgctgcggtcatcgtccgaataaagcgatcgt |
| | | | ctcgaaagaagagatggtggatcggatccgcgcggcggtggatgcgaaaaccgatcctgatttt |
| | | | gtgatcatggcgcgcaccgatgcgctggcggtagaggggctggatgcggcgatcgagcgtgc |
| | | | gcaggcctatgttgaagcgggtgccgaaatgctgttcccggaggcgattaccgaactcgccatgt |
| | | | atcgccagtttgccgatgcggtgcaggtgccgatcctctccaacattaccgaatttggcgcaacac |
| | | | cgctgtttaccaccgacgaattacgcagcgcccatgtcgcaatggcgctctacccgctttcagcgt |
| | | | ttcgcgccatgaaccgcgccgctgaacatgtctataacatcctgcgtcaggaaggcacacagaa |
| | | | aagcgtcatcgacaccatgcagacccgcaacgagctgtacgaaagcatcaactactaccagtac |
| | | | gaagagaagctcgacgacctgtttgcccgtggtcaggtgaaataaaaacgcccgttggttgtattc |
| | | | gacaaccgatgcctgatgcgccgctgacgcgacttatcaggcctacgaggtgaactgaactgta |
| | | | ggtcggataagacgcatagcgtcgcatccgacaacaatctcgaccctacaaatgataacaatga |
| | | | cgaggacaatatgagcgacacaacgatcctgcaaaacagtacccatgtcattaaaccgaaaaaa |
| | | | tcggtggcactttccggcgttccggcgggcaatacggcgctctgcaccgtgggtaaaagcggca |
| | | | acgacctgcattaccgtggctacgatattcttgatctggcggaacattgtgaatttgaagaagtggc |
| | | | gcacctgctgatccacggcaaactgccaacccgtgacgaactcgccgcctacaaaacgaaact |
| | | | gaaagccctgcgtggtttaccggctaacgtgcgtaccgtgctggaagccttaccggcggcgtca |
| | | | cacccgatggatgttatgcgcaccggcgtttccgcgctcggctgcacgctgccagaaaaagagg |
| | | | ggcacaccgtttctggtgcgcgggatattgccgacaaactgctggcgtcacttagttcgattcttct |
| | | | ctactggtatcactacagccacaacggcgaacgcatccagccggaaactgatgacgactctatc |
| | | | ggcggtcacttcctgcatctgctgcacggcgaaaagccgtcgcaaagctgggaaaaggcgatg |
| | | | catatctcgctggtgctgtacgccgaacacgagtttaacgcttccacctttaccagccgggtgattg |
| | | | cgggcactggctctgatatgtattccgccattattggcgcgattggcgcactgcgcgggccgaaa |
| | | | cacggcggggcgaatgaagtgtcgctggagatccagcaacgctacgaaacgccgggcgaag |
| | | | ccgaagccgatatccgcaagcgggtggaaaacaaagaagtggtcattggttttgggcatccggtt |
| | | | tataccatcgccgacccgcgtcatcaggtgatcaaacgtgtggcgaagcagctctcgcaggaag |
| | | | gcggctcgctgaagatgtacaacatcgccgatcgcctggaaacggtgatgtgggagagcaaaa |
| | | | agatgttccccaatctcgactggttctccgctgtttcctacaacatgatgggtgttcccaccgagatg |
| | | | ttcacaccactgtttgttatcgcccgcgtcactggctgggcggcgcacattatcgaacaacgtcag |
| | | | gacaacaaaattatccgtccttccgccaattatgttggaccggaagaccgccagtttgtcgcgctg |
| | | | gataagcgccagtaaacctctacgaataacaataaggaaacgtacccaatgtcagctcaaatcaa |
| | | | caacatccgcccggaatttgatcgtgaaatcgttgatatcgtcgattacgtgatgaactacgaaatc |
| | | | agctccagagtagcctacgacaccgctcattactgcctgcttgacacgctcggctgcggtctgga |
| | | | agctctcgaatatccggcctgtaaaaaactgctggggccaattgtccccggcaccgtcgtaccca |
| | | | acggcgtgcgcgttcccggaactcagtttcagctcgaccccgtccaggcggcatttaacattggc |
| | | | gcgatgatccgttggctcgatttcaacgatacctggctggcggcggagtgggggcatccttccga |
| | | | caacctcggcggcattctggcaacggcggactggctttcgcgcaacgcgatcgccagcggcaa |
| | | | agcgccgttgaccatgaaacaggtgctgaccggaatgatcaaagcccatgaaattcagggctgc |
| | | | atcgcgctggaaaactcctttaaccgcgttggtctcgaccacgttctgttagtgaaagtggcttcca |
| | | | ccgccgtggtcgccgaaatgctcggcctgacccgcgaggaaattctcaacgccgtttcgctggc |
| | | | atgggtagacggacagtcgctgcgcacttatcgtcatgcaccgaacaccggtacgcgtaaatcct |
| | | | gggcggcgggcgatgctacatcccgcgcggtacgtctggcgctgatggcgaaaacgggcgaa |
| | | | atgggttacccgtcagccctgaccgcgccggtgtggggtttctacgacgtctcctttaaaggtgag |
| | | | tcattccgcttccagcgtccgtacggttcctacgtcatggaaaatgtgctgttcaaaatctccttccc |
| | | | ggcggagttccactcccagacggcagttgaagcggcgatgacgctctatgaacagatgcaggc |
| | | | agcaggcaaaacggcggcagatatcgaaaaagtgaccattcgcacccacgaagcctgtattcg |
| | | | catcatcgacaaaaaagggccgctcaataacccggcagaccgcgaccactgcattcagtacatg |
| | | | gtggcgatcccgctgctgttcggacgcttaacggcggcagattacgaggacaacgttgcgcaag |
| | | | ataaacgcatcgacgccctgcgcgagaagatcaattgctttgaagatccggcgtttaccgctgact |
| | | | accacgacccggaaaaacgcgccatcgccaatgccataacccttgagttcaccgacggcacac |
| | | | gatttgaagaagtggtggtggagtacccaattggtcatgctcgccgccgtcaggatggcattccg |
| | | | aagctggtcgataaattcaaaatcaatctcgcgcgccagttcccgactcgccagcagcagcgcat |
| | | | tctggaggtttctctcgacagaactcgcctggaacagatgccggtcaatgagtatctcgacctgta |
| | | | cgtcatttaa |
| 84 | prpBCD | Salmonella | ATGACGTTACACTCACCGGGTCAGGCGTTTCGCGCTGCGC |
| | | | TTGCTAAAGAAAAACCATTACAAATTGTCGGCGCTATCAA |
| | | | CGCCAATCATGCTCTGTTAGCCCAGAGGGCTGGGTATCAG |
| | | | GCTCTCTATCTCTCGGGCGGCGGTGTTGCCGCAGGCTCGCT |
| | | | GGGGCTACCGGATCTGGGCATCTCCACCCTTGATGACGTA |
| | | | TTGACCGATATCCGCCGTATCACCGACGTCTGCCCGCTGCC |
| | | | GCTGCTGGTGGATGCCGATATTGGCTTCGGATCGTCGGCG |
| | | | TTTAACGTAGCGCGTACCGTGAAATCGATTTCCAAAGCCG |
| | | | GCGCCGCCGCGCTGCATATTGAAGATCAGATTGGCGCCAA |
| | | | GCGCTGCGGGCATCGGCCAAATAAAGCGATCGTCTCGAAA |
| | | | GAAGAGATGGTGGACCGGATCCACGCGGCGGTGGATGCG |
| | | | CGGACCGATCCTGACTTTGTCATTATGGCGCGTACCGATG |
| | | | CGCTGGCGGTTGAAGGCCTTGATGCCGCTATCGATCGCGC |
| | | | GCGGGCCTACGTAGAGGCCGGTGCCGACATGCTGTTCCCG |
| | | | GAGGCGATTACTGAACTTGCGATGTACCGCCAGTTTGCCG |
| | | | ACGCAGTGCAGGTGCCAATCCTTGCCAATATTACCGAATT |
| | | | CGGCGCGACGCCGTTGTTTACTACCGAAGAGCTACGCAAC |
| | | | GCCAACGTGGCGATGGCGCTCTATCCGCTGTCGGCGTTCC |
| | | | GGGCGATGAATCGCGCGGCGGAGAAGGTTTACAACGTGCT |
| | | | GCGACAGGAAGGAACGCAAAAGAGCGTTATCGACATCAT |
| | | | GCAGACCCGTAATGAGCTGTATGAAAGCATCAATTATTAC |
| | | | CAGTTCGAGGAAAAACTTGACGCGCTGTACGCCAAAAAAT |
| | | | CGTAGGCCACGGGTCTGATAAAGCGTAGCCGCTATCAAGT |
| | | | CTGTGGCGGACAACCTCAATACCCTACACATTACAAAAAT |
| | | | GACGAGGACACTATGAGCGACACGACGATCCTGCAAAAC |
| | | | AACACAAATGTCATTAAGCCAAAAAAATCCGTCGCATTAT |
| | | | CCGGCGTACCCGCCGGAAATACCGCCTTATGCACCGTAGG |
| | | | TAAAAGCGGTAACGATCTGCACTATCGCGGGTACGATATT |
| | | | CTCGATCTCGCGGAGCACTGTGAATTTGAAGAAGTTGCGC |
| | | | ATCTGCTCATTCACGGCAAGCTGCCCACCCGTGATGAGCT |
| | | | GAATGCCTATAAAAGCAAATTAAAAGCGCTGCGTGGCTTA |
| | | | CCCGCTAACGTCCGTACCGTGCTGGAAGCGCTGCCAGCGG |
| | | | CATCGCACCCGATGGACGTAATGCGCACCGGCGTTTCTGC |
| | | | GCTGGGCTGCACCCTGCCGGAAAAAGAGGGGCATACCGTT |
| | | | TCTGGCGCGCGTGATATCGCCGACAAGCTGCTGGCCTCCC |
| | | | TCAGCTCCATTCTCCTTTACTGGTATCACTACAGCCACAAC |
| | | | GGCGAACGCATTCAGCCAGAAACTGACGATGACTCTATCG |
| | | | GCGGGCATTTCCTGCATTTATTACACGGCGAAAAGCCATC |
| | | | GCAAAGCTGGGAAAAGGCGATGCACATTTCACTGGTACTG |
| | | | TACGCCGAACATGAGTTCAACGCCTCAACCTTTACCAGCC |
| | | | GGGTGGTAGCCGGTACGGGATCGGATATGTACTCCGCCAT |
| | | | CATTGGCGCGATAGGCGCGCTTCGCGGGCCGAAGCACGGC |
| | | | GGGGCGAATGAAGTCTCGCTGGAGATTCAGCAGCGCTACG |
| | | | AAACGCCGGATGAAGCAGAAGCCGATATCCGTAAACGTA |
| | | | TCGCCAATAAAGAAGTGGTGATTGGTTTTGGTCATCCGGT |
| | | | ATACACCATCGCCGATCCGCGCCATCAGGTGATTAAGCGG |
| | | | GTAGCGAAGCAGCTTTCACAGGAGGGCGGTTCGCTGAAGA |
| | | | TGTACAACATTGCCGATCGGCTGGAGACGGTAATGTGGGA |
| | | | CAGCAAAAAGATGTTCCCTAATCTCGACTGGTTCTCGGCG |
| | | | GTCTCCTACAACATGATGGGCGTTCCCACCGAAATGTTTA |
| | | | CCCCGCTGTTTGTGATTGCCCGCGTTACAGGTTGGGCGGC |
| | | | GCACATCATCGAGCAACGACAGGACAACAAAATTATCCGT |
| | | | CCTTCCGCCAATTATATTGGCCCGGAAGATCGCGCCTTTAC |
| | | | GCCGCTGGAACAGCGTCAGTAAACCCTTACCTCTAACGAT |
| | | | AAAAAGGAGTTGCACCCTATGTCCGCACCTGTTTCGAACG |
| | | | TCCGCCCTGAATTTGACCGTGAAATTGTTGATATTGTTGAT |
| | | | TATGTGATGAAGTACAACATCACCTCAAAAGTGGCTTATG |
| | | | ACACCGCGCACTACTGTCTGCTTGATACCCTGGGCTGTGG |
| | | | GCTGGAAGCGCTGGAATATCCGGCCTGTAAAAAATTGATG |
| | | | GGGCCTATCGTGCCAGGTACCGTGGTGCCGAACGGTGTAC |
| | | | GTGTACCGGGCACTCAGTTCCAGCTCGATCCGGTGCAGGC |
| | | | GGCATTTAATATTGGCGCGATGATCCGCTGGCTCGACTTTA |
| | | | ACGATACCTGGCTTGCCGCTGAGTGGGGACACCCTTCCGA |
| | | | TAACCTCGGCGGTATTCTGGCGACCGCCGACTGGTTGTCG |
| | | | CGCAACGCCGTCGCCGCCGGTAAAGCGCCGCTGACCATGC |
| | | | AGCAGGTGCTGACCGGGATGATCAAAGCCCACGAAATCC |
| | | | AGGGCTGTATCGCGCTGGAAAACTCGTTTAACCGCGTGGG |
| | | | TCTCGATCACGTTTTGCTGGTGAAAGTGGCTTCCACGGCTG |
| | | | TAGTGGCTGAAATGCTCGGCCTGACCCGCGATGAAATTCT |
| | | | CAACGCCGTATCGCTGGCGTGGGTGGATGGGCAGTCGCTG |
| | | | CGTACCTATCGCCATGCGCCAAACACCGGTACGCGCAAAT |
| | | | CCTGGGCGGCAGGCGATGCCACTTCACGCGCGGTGCGTCT |
| | | | GGCGCTGATGGCGAAAACTGGCGAGATGGGCTATCCCTCG |
| | | | GCGTTGACCGCCAAAACCTGGGGCTTTTATGACGTCTCGTT |
| | | | CAAAGGCGAAAAATTCCGTTTCCAGCGCCCGTACGGCTCC |
| | | | TACGTGATGGAAAACGTGCTGTTCAAAATCTCCTTCCCGG |
| | | | CGGAGTTCCATTCGCAGACCGCCGTTGAAGCAGCGATGAC |
| | | | GCTGTATGAGCAGATGCAGGCGGCTGGAAAAACGGCGGC |
| | | | GGATATCGAAAAAGTAACGATTCGCACCCATGAAGCCTGT |
| | | | ATACGCATCATTGATAAAAAAGGCCCGCTGAATAATCCGG |
| | | | CTGACCGCGATCACTGTATTCAGTATATGGTGGCGATCCC |
| | | | ACTGCTGTTCGGACGCTTAACGGCGGCGGATTATGAGGAT |
| | | | GGCGTGGCGCAGGATAAACGTATTGACGCGCTGCGTGAAA |
| | | | AAACGCATTGCTTTGAAGACCCGGCGTTTACCACTGATTA |
| | | | TCATGACCCGGAAAAACGTTCGATTGCCAACGCCATTAGT |
| | | | CTTGAATTTACTGACGGTACCCGTTTTGACGAGGTGGTTGT |
| | | | CGAGTACCCGATCGGCCACGCGCGTCGTCGCGGCGACGGC |
| | | | ATTCCAAAACTTATCGAAAAATTTAAAATCAATCTGGCGC |
| | | | GCCAGTTCCCACCCCGCCAGCAACAACGCATCCTGGATGT |
| | | | CTCCCTGGACAGAACGCGCCTGGAGCAGATGCCGGTTAAT |
| | | | GAGTATCTCGACTTGTACGTCATCTAG |
| 85 | PrpR | E. coli | MAHPPRLNDDKPVIWTVSVTRLFELFRDISLEFDHLANITPIQ |
| | (polypeptide) | | LGFEKAVAYIRKKLASERCDAIIAAGSNGAYLKSRLSVPVILI |
| | | | KPSGYDVLQALAKAGKLTSSIGVVTYQETIPALVAFQKTFNL |
| | | | RLDQRSYITEEDARGQINELKANGTEAVVGAGLITDLAEEAG |
| | | | MTGIFIYSAATVRQAFSDALDMTRMSLRHNTHDATRNALRT |
| | | | RYVLGDMLGQSPQMEQVRQTILLYARSSAAVLIEGETGTGK |
| | | | ELAAQAIHREYFARHDVRQGKKSHPFVAVNCGAIAESLLEAE |
| | | | LFGYEEGAFTGSRRGGRAGLFEIAHGGTLFLDEIGEMPLPLQT |
| | | | RLLRVLEEKEVTRVGGHQPVPVDVRVISATHCNLEEDMQQG |
| | | | QFRRDLFYRLSILRLQLPPLRERVADILPLAESFLKMSLAALS |
| | | | VPFSAALRQGLETCQIVLLLYDWPGNIRELRNMMERLALFLS |
| | | | VEPTPDLTPQFLQLLLPELARESAKTPIPGLLTAQQALEKFNG |
| | | | DKTAAANYLGISRTTFWRRLKS |
| 86 | prpR | E. coli | tcagcttttcagccgccgccagaacgtcgtccggctgatacctaaataattcgccgctgctgtctta |
| | | | tcgccattaaatttctccagtgcctgttgtgctgtcagcaagcctggaatgggagtcttcgccgact |
| | | | cgcgcgccagttccggcagtagcagctgcaaaaattgcggcgttaaatccggcgtcggttccac |
| | | | acttaaaaataacgccagtcgttccatcatattgcgcagttcacgaatattgcccggccagtcgtag |
| | | | agcaataatacaatctgacaggtctctaatccctgacgtaatgcggcagaaaaagggacagaga |
| | | | gtgccgccagagacattttcaaaaagctttccgccagcggcagaatatccgccacccgctcgcgt |
| | | | agcggcggcagttgcaggcgtaaaatactcagccgataaaacaggtcacggcgaaactgccct |
| | | | tgctgcatatcttcttccagattgcagtgagtggcgctaatgacccgcacatctaccggaacaggc |
| | | | tgatgcccgccgacgcgggtgacctctttttcttccagcacccgtaacaggcgagtctgcaacgg |
| | | | cagcggcatttcgccaatctcatccagaaacagcgtaccgccgtgggcaatttcgaacagcccg |
| | | | gcgcgacctccgcgtcgcgagccggtgaacgccccttcctcatagccaaacagctctgcttcca |
| | | | gcagcgattcggcaatcgccccgcagttgacggcaacaaacggatgtgactttttgccctgtcgc |
| | | | acatcgtggcgggcaaaatattctcgatgaatcgcctgggccgccagctctttgcccgtccccgtt |
| | | | tccccctcaatcaacaccgccgcactggagcgggcatacagcaaaatagtctgccgcacctgtt |
| | | | ccatctgtggtgattgaccgagcatatcgcccagcacgtaacgagtacgcagggcgttgcgggt |
| | | | ggcatcgtgagtgttatggcgtaacgacatgcgcgtcatatccagcgcatcgctaaatgcctggc |
| | | | gcacggtggcggcagaatagataaaaattccggtcattccggcttcttctgccagatcggtaatca |
| | | | gccccgcgccgaccaccgcttcggtgccgttggcttttagctcgttaatctgcccgcgtgcgtctt |
| | | | cttcggtaatgtagctacgttggtcgaggcgcaaattaaaggttttttgaaacgctaccagtgccgg |
| | | | aatggtttcctgataggtgacaacgccgatagaagaggtgagttttccggcttttgccagcgcctgt |
| | | | aacacatcgtagccactcggttttatcagaatcaccggtaccgacaggcggcttttcaggtacgca |
| | | | ccgttagagccagcggcaatgatggcgtcgcagcgttcgctggccagttttttgcggatgtaggc |
| | | | caccgctttttcaaagccaagctgaataggggtgatgttcgccagatgatcaaactcgaggctgat |
| | | | atcgcgaaacagctcgaacagccgcgttacagataccgtccagataaccggtttgtcgtcattca |
| | | | gccgtggtggatgtgccat |
| 87 | MctC | Corynebacterium | MNSTILLAQDAVSEGVGNPILNISVFVVFIIVTMTVVLRVGKS |
| | (polypeptide) | | TSESTDFYTGGASFSGTQNGLAIAGDYLSAASFLGIVGAISLN |
| | | | GYDGFLYSIGFFVAWLVALLLVAEPLRNVGRFTMADVLSFR |
| | | | LRQKPVRVAAACGTLAVTLFYLIAQMAGAGSLVSVLLDIHEF |
| | | | KWQAVVVGIVGIVMIAYVLLGGMKGTTYVQMIKAVLLVGG |
| | | | VAIMTVLTFVKVSGGLTTLLNDAVEKHAASDYAATKGYDPT |
| | | | QILEPGLQYGATLTTQLDFISLALALCLGTAGLPHVLMRFYT |
| | | | VPTAKEARKSVTWAIVLIGAFYLMTLVLGYGAAALVGPDRV |
| | | | IAAPGAANAAAPLLAFELGGSIFMALISAVAFATVLAVVAGL |
| | | | AITASAAVGHDIYNAVIRNGQSTEAEQVRVSRITVVVIGLISIV |
| | | | LGILAMTQNVAFLVALAFAVAASANLPTILYSLYWKKFNTT |
| | | | GAVAAIYTGLISALLLIFLSPAVSGNDSAMVPGADWAIFPLKN |
| | | | PGLVSIPLAFIAGWIGTLVGKPDNMDDLAAEMEVRSLTGVGV |
| | | | EKAVDH |
| 88 | mctC | Corynebacterium | ATGAATTCCACTATTCTCCTTGCACAAGACGCTGTTTCTGA |
| | | | GGGCGTCGGTAATCCGATTCTTAACATCAGTGTCTTCGTCG |
| | | | TCTTCATTATTGTGACGATGACCGTGGTGCTTCGCGTGGGC |
| | | | AAGAGCACCAGCGAATCCACCGACTTCTACACCGGTGGTG |
| | | | CTTCCTTCTCCGGAACCCAGAACGGTCTGGCTATCGCAGG |
| | | | TGACTACCTGTCTGCAGCGTCCTTCCTCGGAATCGTTGGTG |
| | | | CAATTTCACTCAACGGTTACGACGGATTCCTTTACTCCATC |
| | | | GGCTTCTTCGTCGCATGGCTTGTTGCACTGCTGCTCGTGGC |
| | | | AGAGCCACTTCGTAACGTGGGCCGCTTCACCATGGCTGAC |
| | | | GTGCTGTCCTTCCGACTGCGTCAGAAACCAGTCCGCGTCG |
| | | | CTGCGGCCTGCGGTACCCTCGCGGTTACCCTCTTTTACTTG |
| | | | ATCGCTCAGATGGCTGGTGCAGGTTCGCTTGTGTCCGTTCT |
| | | | GCTGGACATCCACGAGTTCAAGTGGCAGGCAGTTGTTGTC |
| | | | GGTATCGTTGGCATTGTCATGATCGCCTACGTTCTTCTTGG |
| | | | CGGTATGAAGGGCACCACATACGTTCAGATGATTAAGGCA |
| | | | GTTCTGCTGGTCGGTGGCGTTGCCATTATGACCGTTCTGAC |
| | | | CTTCGTCAAGGTGTCTGGTGGCCTGACCACCCTTTTAAATG |
| | | | ACGCTGTTGAGAAGCACGCCGCTTCAGATTACGCTGCCAC |
| | | | CAAGGGGTACGATCCAACCCAGATCCTGGAGCCTGGTCTG |
| | | | CAGTACGGTGCAACTCTGACCACTCAGCTGGACTTCATTTC |
| | | | CTTGGCTCTCGCTCTGTGTCTTGGAACCGCTGGTCTGCCAC |
| | | | ACGTTCTGATGCGCTTCTACACCGTTCCTACCGCCAAGGA |
| | | | AGCACGTAAGTCTGTGACCTGGGCTATCGTCCTCATTGGT |
| | | | GCGTTCTACCTGATGACCCTGGTCCTTGGTTACGGCGCTGC |
| | | | GGCACTGGTCGGTCCAGACCGCGTCATTGCCGCACCAGGT |
| | | | GCTGCTAATGCTGCTGCTCCTCTGCTGGCCTTCGAGCTTGG |
| | | | TGGTTCCATCTTCATGGCGCTGATTTCCGCAGTTGCGTTCG |
| | | | CTACCGTTCTCGCCGTGGTCGCAGGTCTTGCAATTACCGCA |
| | | | TCCGCTGCTGTTGGTCACGACATCTACAACGCTGTTATCCG |
| | | | CAACGGTCAGTCCACCGAAGCGGAGCAGGTCCGAGTATCC |
| | | | CGCATCACCGTTGTCGTCATTGGCCTGATTTCCATTGTCCT |
| | | | GGGAATTCTTGCAATGACCCAGAACGTTGCGTTCCTCGTG |
| | | | GCCCTGGCCTTCGCAGTTGCAGCATCCGCTAACCTGCCAA |
| | | | CCATCCTGTACTCCCTGTACTGGAAGAAGTTCAACACCAC |
| | | | CGGCGCTGTGGCCGCTATCTACACCGGTCTCATCTCCGCGC |
| | | | TGCTGCTGATCTTCCTGTCCCCAGCAGTCTCCGGTAATGAC |
| | | | AGCGCAATGGTTCCAGGTGCAGACTGGGCAATCTTCCCAC |
| | | | TGAAGAACCCAGGCCTCGTCTCCATCCCACTGGCATTCAT |
| | | | CGCTGGTTGGATCGGCACTTTGGTTGGCAAGCCAGACAAC |
| | | | ATGGATGATCTTGCTGCCGAAATGGAAGTTCGTTCCCTCA |
| | | | CCGGTGTCGGTGTTGAAAAGGCTGTTGATCACTAA |
| 89 | PutP_6 | Virgibacillus | MDLTTLITFIVYLLGMLAIGLIMYYRTNNLSDYVLGGRDLGP |
| | (polypeptide) | sp. | GVAALSAGASDMSGWLLLGLPGAIYASGMSEAWMGIGLAV |
| | | | GAYLNWQFVAKRLRVYTEVSNNSITIPDYFENRFKDNSHILR |
| | | | VISAIVILLFFTFYTSSGMVAGAKLFEASFGLQYETALWIGAV |
| | | | VVVSYTLLGGFLAVAWTDFIQGILMFLALIVVPIVALDQMGG |
| | | | WNQAVQAVGEINPSHLNMVEGVGIMAIISSLAWGLGYFGQP |
| | | | HIIVRFMALRSAKDVPKAKFIGTAWMILGLYGAIFTGFVGLA |
| | | | FISTQEVPILSEFGIQVVNENGLQMLADPEKIFIAFSQILFHPVV |
| | | | AGILLAAILSAIMSTVDSQLLVSSSAVAEDFYKAIFRKKATGK |
| | | | ELVWVGRIATVIIAIVALIIAMNPDSSVLDLVSYAWAGFGAAF |
| | | | GPIIILSLFWKRITRNGALAGIIVGAITVIVWGDFLSGGIFDLYE |
| | | | IVPGFILNMIVTVIVSLIDKPNPDLEADFDETVEKMKE |
| 90 | putP_6 | Virgibacillus | atggatcttacgacattaataacttttatagtatatctactagggatgttggcgattggcctcatcat |
| | | sp. | gtattatcgaaccaataatttatcagattatgttcttggtggacgtgatcttggtccaggcgtagc |
| | | | tgcattgagtgctggtgcatcggatatgagtggttggctgttattaggtttgcctggagcgatttatg |
| | | | catctggtatgtctgaagcttggatggggatcgggttagctgtaggtgcttatttaaattggcaattt |
| | | | gtagctaagcgattacgcgtttataccgaggtatcaaataattccattacgatcccagattattttg |
| | | | aaaatcggtttaaagataactcacatattcttcgtgttatatctgctatcgtaattttgttattcttc |
| | | | actttttatacatcttcaggaatggttgcaggagcaaaattatttgaggcttcattcggtctccaata |
| | | | cgaaactgctctgtggattggtgcggttgtagttgtatcttatacgttacttggaggatttctagcgg |
| | | | ttgcatggacagactttattcaaggtattcttatgttccttgcactaattgttgttccaatcgtcgca |
| | | | ttagatcaaatgggtggctggaatcaagcggtacaagctgttggtgaaattaatccttcccacc |
| | | | tcaatatggttgaaggtgttggaataatggcaattatttcatcacttgcttggggcttaggttattt |
| | | | tggacagccacatattattgttcgttttatggcattacgttcggcgaaagatgttccgaaagcg |
| | | | aaatttattggaacagcttggatgattttaggactttatggagcaatctttactggttttgtagg |
| | | | actagcatttatcagtacacaagaagtaccgattctgtctgaattcgggattcaagtagtt |
| | | | aatgagaatggtttacaaatgttagccgatcctgaaaagatatttattgctttctcccaaat |
| | | | actattccatccagtagttgccggtatcttactagcggcaatcttgtctgcaattatgagta |
| | | | ccgttgattcacagttacttgtatcatcttcagcggttgcagaagatttctataaagctattt |
| | | | tccgtaaaaaagctactggtaaagagcttgtttgggttggacgtattgctacagtgataattgc |
| | | | gattgttgctttaattattgcaatgaacccagatagctctgtattggatctagttagttatgcatggg |
| | | | ctggatttggtgcagcatttggaccaattatcatcttgtcattattctggaagagaatcacaagaaat |
| | | | ggtgcactagcgggtatcattgtaggtgccattacggtaattgtatggggagactttctatctggagg |
| | | | tatctttgacctctacgaaattgttccaggctttatcttaaatatgattgtcaccgttattgtgagtc |
| | | | ttatcgataaaccgaatccagatttagaagctgactttgatgaaaccgtagaaaaaatgaaagaataa |
| 91 | MhpT | E. coli | MSTRTPSSSSSRLMLTIGLCFLVALMEGLDLQAAGIAAGGIAQ |
| | (polypeptide) | | AFALDKMQMGWIFSAGILGLLPGALVGGMLADRYGRKRILI |
| | | | GSVALFGLFSLATAIAWDFPSLVFARLMTGVGLGAALPNLIA |
| | | | LTSEAAGPRFRGTAVSLMYCGVPIGAALAATLGFAGANLAW |
| | | | QTVFWVGGVVPLILVPLLMRWLPESAVFAGEKQSAPPLRALF |
| | | | APETATATLLLWLCYFFTLLVVYMLINWLPLLLVEQGFQPSQ |
| | | | AAGVMFALQMGAASGTLMLGALMDKLRPVTMSLLIYSGML |
| | | | ASLLALGTVSSFNGMLLAGFVAGLFATGGQSVLYALAPLFYS |
| | | | SQIRATGVGTAVAVGRLGAMSGPLLAGKMLALGTGTVGVM |
| | | | AASAPGILVAGLAVFILMSRRSRIQPCADA |
| 92 | mhpT | E. coli | atgTCGACTCGTACCCCTTCATCATCTTCATCCCGCCTGATG |
| | | | CTGACCATCGGGCTTTGTTTTTTGGTCGCTCTGATGGAAGG |
| | | | GCTGGATCTTCAGGCGGCTGGCATTGCGGCGGGTGGCATC |
| | | | GCCCAGGCTTTCGCACTCGATAAAATGCAAATGGGCTGGA |
| | | | TATTTAGCGCCGGAATACTCGGTTTGCTACCCGGCGCGTTG |
| | | | GTTGGCGGAATGCTGGCGGACCGTTATGGTCGCAAGCGCA |
| | | | TTTTGATTGGCTCAGTTGCGCTGTTTGGTTTGTTCTCACTGG |
| | | | CAACGGCGATTGCCTGGGATTTCCCCTCACTGGTCTTTGCG |
| | | | CGGCTGATGACCGGTGTCGGGCTGGGGGCGGCGTTGCCGA |
| | | | ATCTTATCGCCCTGACGTCTGAAGCCGCGGGTCCACGTTTT |
| | | | CGTGGGACGGCAGTGAGCCTGATGTATTGCGGTGTTCCCA |
| | | | TTGGCGCGGCGCTGGCGGCGACACTGGGTTTCGCGGGGGC |
| | | | AAACTTAGCATGGCAAACGGTGTTTTGGGTAGGTGGTGTG |
| | | | GTGCCGTTGATTCTGGTGCCGCTATTAATGCGCTGGCTGCC |
| | | | GGAGTCGGCGGTTTTCGCTGGCGAAAAACAGTCTGCGCCA |
| | | | CCACTGCGTGCCTTATTTGCGCCAGAAACGGCAACCGCGA |
| | | | CGCTGCTGCTGTGGTTGTGTTATTTCTTCACTCTGCTGGTG |
| | | | GTCTACATGTTGATCAACTGGCTACCGCTACTTTTGGTGGA |
| | | | GCAAGGATTCCAGCCATCGCAGGCGGCAGGGGTGATGTTT |
| | | | GCCCTGCAAATGGGGGCGGCAAGCGGGACGTTAATGTTGG |
| | | | GCGCATTGATGGATAAGCTGCGTCCAGTAACCATGTCGCT |
| | | | ACTGATTTATAGCGGCATGTTAGCTTCGCTGCTGGCGCTTG |
| | | | GAACGGTGTCGTCATTTAACGGTATGTTGCTGGCGGGATTT |
| | | | GTCGCGGGGTTGTTTGCGACAGGTGGGCAAAGCGTTTTGT |
| | | | ATGCCCTGGCACCGTTGTTTTACAGTTCGCAGATCCGCGCA |
| | | | ACAGGTGTGGGAACAGCCGTGGCGGTAGGGCGTCTGGGG |
| | | | GCTATGAGCGGTCCGTTACTGGCCGGGAAAATGCTGGCAT |
| | | | TAGGCACTGGCACGGTCGGCGTAATGGCCGCTTCTGCACC |
| | | | GGGTATTCTTGTTGCTGGGTTGGCGGTGTTTATTTTGATGA |
| | | | GCCGGAGATCACGAATACAGCCGTGCGCCGATGCCTGA |
| 93 | prpBCDE | E. coli | atgtctctacactctccaggtaaagcgtttcgcgctgcacttagcaaagaaaccccgttgcaaattg |
| | | | ttggcaccatcaacgctaaccatgcgctgctggcgcagcgtgccggatatcaggcgatttatctct |
| | | | ccggcggtggcgtggcggcaggatcgctggggctgcccgatctcggtatttctactcttgatgac |
| | | | gtgctgacagatattcgccgtatcaccgacgtttgttcgctgccgctgctggtggatgcggatatc |
| | | | ggttttggttcttcagcctttaacgtggcgcgtacggtgaaatcaatgattaaagccggtgcggca |
| | | | ggattgcatattgaagatcaggttggtgcgaaacgctgcggtcatcgtccgaataaagcgatcgt |
| | | | ctcgaaagaagagatggtggatcggatccgcgcggcggtggatgcgaaaaccgatcctgatttt |
| | | | gtgatcatggcgcgcaccgatgcgctggcggtagaggggctggatgcggcgatcgagcgtgc |
| | | | gcaggcctatgttgaagcgggtgccgaaatgctgttcccggaggcgattaccgaactcgccatgt |
| | | | atcgccagtttgccgatgcggtgcaggtgccgatcctctccaacattaccgaatttggcgcaacac |
| | | | cgctgtttaccaccgacgaattacgcagcgcccatgtcgcaatggcgctctacccgctttcagcgt |
| | | | ttcgcgccatgaaccgcgccgctgaacatgtctataacatcctgcgtcaggaaggcacacagaa |
| | | | aagcgtcatcgacaccatgcagacccgcaacgagctgtacgaaagcatcaactactaccagtac |
| | | | gaagagaagctcgacgacctgtttgcccgtggtcaggtgaaataaaaacgcccgttggttgtattc |
| | | | gacaaccgatgcctgatgcgccgctgacgcgacttatcaggcctacgaggtgaactgaactgta |
| | | | ggtcggataagacgcatagcgtcgcatccgacaacaatctcgaccctacaaatgataacaatga |
| | | | cgaggacaatatgagcgacacaacgatcctgcaaaacagtacccatgtcattaaaccgaaaaaa |
| | | | tcggtggcactttccggcgttccggcgggcaatacggcgctctgcaccgtgggtaaaagcggca |
| | | | acgacctgcattaccgtggctacgatattcttgatctggcggaacattgtgaatttgaagaagtggc |
| | | | gcacctgctgatccacggcaaactgccaacccgtgacgaactcgccgcctacaaaacgaaact |
| | | | gaaagccctgcgtggtttaccggctaacgtgcgtaccgtgctggaagccttaccggcggcgtca |
| | | | cacccgatggatgttatgcgcaccggcgtttccgcgctcggctgcacgctgccagaaaaagagg |
| | | | ggcacaccgtttctggtgcgcgggatattgccgacaaactgctggcgtcacttagttcgattcttct |
| | | | ctactggtatcactacagccacaacggcgaacgcatccagccggaaactgatgacgactctatc |
| | | | ggcggtcacttcctgcatctgctgcacggcgaaaagccgtcgcaaagctgggaaaaggcgatg |
| | | | catatctcgctggtgctgtacgccgaacacgagtttaacgcttccacctttaccagccgggtgattg |
| | | | cgggcactggctctgatatgtattccgccattattggcgcgattggcgcactgcgcgggccgaaa |
| | | | cacggcggggcgaatgaagtgtcgctggagatccagcaacgctacgaaacgccgggcgaag |
| | | | ccgaagccgatatccgcaagcgggtggaaaacaaagaagtggtcattggttttgggcatccggtt |
| | | | tataccatcgccgacccgcgtcatcaggtgatcaaacgtgtggcgaagcagctctcgcaggaag |
| | | | gcggctcgctgaagatgtacaacatcgccgatcgcctggaaacggtgatgtgggagagcaaaa |
| | | | agatgttccccaatctcgactggttctccgctgtttcctacaacatgatgggtgttcccaccgagatg |
| | | | ttcacaccactgtttgttatcgcccgcgtcactggctgggcggcgcacattatcgaacaacgtcag |
| | | | gacaacaaaattatccgtccttccgccaattatgttggaccggaagaccgccagtttgtcgcgctg |
| | | | gataagcgccagtaaacctctacgaataacaataaggaaacgtacccaatgtcagctcaaatcaa |
| | | | caacatccgcccggaatttgatcgtgaaatcgttgatatcgtcgattacgtgatgaactacgaaatc |
| | | | agctccagagtagcctacgacaccgctcattactgcctgcttgacacgctcggctgcggtctgga |
| | | | agctctcgaatatccggcctgtaaaaaactgctggggccaattgtccccggcaccgtcgtaccca |
| | | | acggcgtgcgcgttcccggaactcagtttcagctcgaccccgtccaggcggcatttaacattggc |
| | | | gcgatgatccgttggctcgatttcaacgatacctggctggcggcggagtgggggcatccttccga |
| | | | caacctcggcggcattctggcaacggcggactggctttcgcgcaacgcgatcgccagcggcaa |
| | | | agcgccgttgaccatgaaacaggtgctgaccggaatgatcaaagcccatgaaattcagggctgc |
| | | | atcgcgctggaaaactcctttaaccgcgttggtctcgaccacgttctgttagtgaaagtggcttcca |
| | | | ccgccgtggtcgccgaaatgctcggcctgacccgcgaggaaattctcaacgccgtttcgctggc |
| | | | atgggtagacggacagtcgctgcgcacttatcgtcatgcaccgaacaccggtacgcgtaaatcct |
| | | | gggcggcgggcgatgctacatcccgcgcggtacgtctggcgctgatggcgaaaacgggcgaa |
| | | | atgggttacccgtcagccctgaccgcgccggtgtggggtttctacgacgtctcctttaaaggtgag |
| | | | tcattccgcttccagcgtccgtacggttcctacgtcatggaaaatgtgctgttcaaaatctccttccc |
| | | | ggcggagttccactcccagacggcagttgaagcggcgatgacgctctatgaacagatgcaggc |
| | | | agcaggcaaaacggcggcagatatcgaaaaagtgaccattcgcacccacgaagcctgtattcg |
| | | | catcatcgacaaaaaagggccgctcaataacccggcagaccgcgaccactgcattcagtacatg |
| | | | gtggcgatcccgctgctgttcggacgcttaacggcggcagattacgaggacaacgttgcgcaag |
| | | | ataaacgcatcgacgccctgcgcgagaagatcaattgctttgaagatccggcgtttaccgctgact |
| | | | accacgacccggaaaaacgcgccatcgccaatgccataacccttgagttcaccgacggcacac |
| | | | gatttgaagaagtggtggtggagtacccaattggtcatgctcgccgccgtcaggatggcattccg |
| | | | aagctggtcgataaattcaaaatcaatctcgcgcgccagttcccgactcgccagcagcagcgcat |
| | | | tctggaggtttctctcgacagaactcgcctggaacagatgccggtcaatgagtatctcgacctgta |
| | | | cgtcatttaagtaaacggcggtaaggcgtaagttcaacaggagagcattatgtcttttagcgaatttt |
| | | | atcagcgttcgattaacgaaccggagaagttctgggccgagcaggcccggcgtattgactggca |
| | | | gacgccctttacgcaaacgctcgaccacagcaacccgccgtttgcccgttggttttgtgaaggcc |
| | | | gaaccaacttgtgtcacaacgctatcgaccgctggctggagaaacagccagaggcgctggcatt |
| | | | gattgccgtctcttcggaaacagaggaagagcgtacctttaccttccgccagttacatgacgaagt |
| | | | gaatgcggtggcgtcaatgctgcgctcactgggcgtgcagcgtggcgatcgggtgctggtgtat |
| | | | atgccgatgattgccgaagcgcatattaccctgctggcctgcgcgcgcattggtgctattcactcg |
| | | | gtggtgtttgggggatttgcttcgcacagcgtggcaacgcgaattgatgacgctaaaccggtgct |
| | | | gattgtctcggctgatgccggggcgcgcggcggtaaaatcattccgtataaaaaattgctcgacg |
| | | | atgcgataagtcaggcacagcatcagccgcgtcacgttttactggtggatcgcgggctggcgaa |
| | | | aatggcgcgcgttagcgggcgggatgtcgatttcgcgtcgttgcgccatcaacacatcggcgcg |
| | | | cgggtgccggtggcatggctggaatccaacgaaacctcctgcattctctacacctccggcacga |
| | | | ccggcaaacctaaaggtgtgcagcgtgatgtcggcggatatgcggtggcgctggcgacctcga |
| | | | tggacaccatttttggcggcaaagcgggcggcgtgttcttttgtgcttcggatatcggctgggtggt |
| | | | agggcattcgtatatcgtttacgcgccgctgctggcggggatggcgactatcgtttacgaaggatt |
| | | | gccgacctggccggactgcggcgtgtggtggaaaattgtcgagaaatatcaggttagccgcatg |
| | | | ttctcagcgccgaccgccattcgcgtgctgaaaaaattccctaccgctgaaattcgcaaacacgat |
| | | | ctttcgtcgctggaagtgctctatctggctggagaaccgctggacgagccgaccgccagttgggt |
| | | | gagcaatacgctggatgtgccggtcatcgacaactactggcagaccgaatccggctggccgatt |
| | | | atggcgattgctcgcggtctggatgacagaccgacgcgtctgggaagccccggcgtgccgatg |
| | | | tatggctataacgtgcagttgctcaatgaagtcaccggcgaaccgtgtggcgtcaatgagaaagg |
| | | | gatgctggtagtggaggggccattgccgccaggctgtattcaaaccatctggggcgacgacgac |
| | | | cgctttgtgaagacgtactggtcgctgttttcccgtccggtgtacgccacttttgactggggcatcc |
| | | | gcgatgctgacggttatcactttattctcgggcgcactgacgatgtgattaacgttgccggacatcg |
| | | | gctgggtacgcgtgagattgaagagagtatctccagtcatccgggcgttgccgaagtggcggtg |
| | | | gttggggtgaaagatgcgctgaaagggcaggtggcggtggcgtttgtcattccgaaagagagc |
| | | | gacagtctggaagaccgtgaggtggcgcactcgcaagagaaggcgattatggcgctggtggac |
| | | | agccagattggcaactttggccgcccggcgcacgtctggtttgtctcgcaattgccaaaaacgcg |
| | | | atccggaaaaatgctgcgccgcacgatccaggcgatttgcgaaggacgcgatcctggggatct |
| | | | gacgaccattgatgatccggcgtcgttggatcagatccgccaggcgatggaagagtag |
| 94 | prpBCDE | Salmonella | ATGACGTTACACTCACCGGGTCAGGCGTTTCGCGCTGCGC |
| | | | TTGCTAAAGAAAAACCATTACAAATTGTCGGCGCTATCAA |
| | | | CGCCAATCATGCTCTGTTAGCCCAGAGGGCTGGGTATCAG |
| | | | GCTCTCTATCTCTCGGGCGGCGGTGTTGCCGCAGGCTCGCT |
| | | | GGGGCTACCGGATCTGGGCATCTCCACCCTTGATGACGTA |
| | | | TTGACCGATATCCGCCGTATCACCGACGTCTGCCCGCTGCC |
| | | | GCTGCTGGTGGATGCCGATATTGGCTTCGGATCGTCGGCG |
| | | | TTTAACGTAGCGCGTACCGTGAAATCGATTTCCAAAGCCG |
| | | | GCGCCGCCGCGCTGCATATTGAAGATCAGATTGGCGCCAA |
| | | | GCGCTGCGGGCATCGGCCAAATAAAGCGATCGTCTCGAAA |
| | | | GAAGAGATGGTGGACCGGATCCACGCGGCGGTGGATGCG |
| | | | CGGACCGATCCTGACTTTGTCATTATGGCGCGTACCGATG |
| | | | CGCTGGCGGTTGAAGGCCTTGATGCCGCTATCGATCGCGC |
| | | | GCGGGCCTACGTAGAGGCCGGTGCCGACATGCTGTTCCCG |
| | | | GAGGCGATTACTGAACTTGCGATGTACCGCCAGTTTGCCG |
| | | | ACGCAGTGCAGGTGCCAATCCTTGCCAATATTACCGAATT |
| | | | CGGCGCGACGCCGTTGTTTACTACCGAAGAGCTACGCAAC |
| | | | GCCAACGTGGCGATGGCGCTCTATCCGCTGTCGGCGTTCC |
| | | | GGGCGATGAATCGCGCGGCGGAGAAGGTTTACAACGTGCT |
| | | | GCGACAGGAAGGAACGCAAAAGAGCGTTATCGACATCAT |
| | | | GCAGACCCGTAATGAGCTGTATGAAAGCATCAATTATTAC |
| | | | CAGTTCGAGGAAAAACTTGACGCGCTGTACGCCAAAAAAT |
| | | | CGTAGGCCACGGGTCTGATAAAGCGTAGCCGCTATCAAGT |
| | | | CTGTGGCGGACAACCTCAATACCCTACACATTACAAAAAT |
| | | | GACGAGGACACTATGAGCGACACGACGATCCTGCAAAAC |
| | | | AACACAAATGTCATTAAGCCAAAAAAATCCGTCGCATTAT |
| | | | CCGGCGTACCCGCCGGAAATACCGCCTTATGCACCGTAGG |
| | | | TAAAAGCGGTAACGATCTGCACTATCGCGGGTACGATATT |
| | | | CTCGATCTCGCGGAGCACTGTGAATTTGAAGAAGTTGCGC |
| | | | ATCTGCTCATTCACGGCAAGCTGCCCACCCGTGATGAGCT |
| | | | GAATGCCTATAAAAGCAAATTAAAAGCGCTGCGTGGCTTA |
| | | | CCCGCTAACGTCCGTACCGTGCTGGAAGCGCTGCCAGCGG |
| | | | CATCGCACCCGATGGACGTAATGCGCACCGGCGTTTCTGC |
| | | | GCTGGGCTGCACCCTGCCGGAAAAAGAGGGGCATACCGTT |
| | | | TCTGGCGCGCGTGATATCGCCGACAAGCTGCTGGCCTCCC |
| | | | TCAGCTCCATTCTCCTTTACTGGTATCACTACAGCCACAAC |
| | | | GGCGAACGCATTCAGCCAGAAACTGACGATGACTCTATCG |
| | | | GCGGGCATTTCCTGCATTTATTACACGGCGAAAAGCCATC |
| | | | GCAAAGCTGGGAAAAGGCGATGCACATTTCACTGGTACTG |
| | | | TACGCCGAACATGAGTTCAACGCCTCAACCTTTACCAGCC |
| | | | GGGTGGTAGCCGGTACGGGATCGGATATGTACTCCGCCAT |
| | | | CATTGGCGCGATAGGCGCGCTTCGCGGGCCGAAGCACGGC |
| | | | GGGGCGAATGAAGTCTCGCTGGAGATTCAGCAGCGCTACG |
| | | | AAACGCCGGATGAAGCAGAAGCCGATATCCGTAAACGTA |
| | | | TCGCCAATAAAGAAGTGGTGATTGGTTTTGGTCATCCGGT |
| | | | ATACACCATCGCCGATCCGCGCCATCAGGTGATTAAGCGG |
| | | | GTAGCGAAGCAGCTTTCACAGGAGGGCGGTTCGCTGAAGA |
| | | | TGTACAACATTGCCGATCGGCTGGAGACGGTAATGTGGGA |
| | | | CAGCAAAAAGATGTTCCCTAATCTCGACTGGTTCTCGGCG |
| | | | GTCTCCTACAACATGATGGGCGTTCCCACCGAAATGTTTA |
| | | | CCCCGCTGTTTGTGATTGCCCGCGTTACAGGTTGGGCGGC |
| | | | GCACATCATCGAGCAACGACAGGACAACAAAATTATCCGT |
| | | | CCTTCCGCCAATTATATTGGCCCGGAAGATCGCGCCTTTAC |
| | | | GCCGCTGGAACAGCGTCAGTAAACCCTTACCTCTAACGAT |
| | | | AAAAAGGAGTTGCACCCTATGTCCGCACCTGTTTCGAACG |
| | | | TCCGCCCTGAATTTGACCGTGAAATTGTTGATATTGTTGAT |
| | | | TATGTGATGAAGTACAACATCACCTCAAAAGTGGCTTATG |
| | | | ACACCGCGCACTACTGTCTGCTTGATACCCTGGGCTGTGG |
| | | | GCTGGAAGCGCTGGAATATCCGGCCTGTAAAAAATTGATG |
| | | | GGGCCTATCGTGCCAGGTACCGTGGTGCCGAACGGTGTAC |
| | | | GTGTACCGGGCACTCAGTTCCAGCTCGATCCGGTGCAGGC |
| | | | GGCATTTAATATTGGCGCGATGATCCGCTGGCTCGACTTTA |
| | | | ACGATACCTGGCTTGCCGCTGAGTGGGGACACCCTTCCGA |
| | | | TAACCTCGGCGGTATTCTGGCGACCGCCGACTGGTTGTCG |
| | | | CGCAACGCCGTCGCCGCCGGTAAAGCGCCGCTGACCATGC |
| | | | AGCAGGTGCTGACCGGGATGATCAAAGCCCACGAAATCC |
| | | | AGGGCTGTATCGCGCTGGAAAACTCGTTTAACCGCGTGGG |
| | | | TCTCGATCACGTTTTGCTGGTGAAAGTGGCTTCCACGGCTG |
| | | | TAGTGGCTGAAATGCTCGGCCTGACCCGCGATGAAATTCT |
| | | | CAACGCCGTATCGCTGGCGTGGGTGGATGGGCAGTCGCTG |
| | | | CGTACCTATCGCCATGCGCCAAACACCGGTACGCGCAAAT |
| | | | CCTGGGCGGCAGGCGATGCCACTTCACGCGCGGTGCGTCT |
| | | | GGCGCTGATGGCGAAAACTGGCGAGATGGGCTATCCCTCG |
| | | | GCGTTGACCGCCAAAACCTGGGGCTTTTATGACGTCTCGTT |
| | | | CAAAGGCGAAAAATTCCGTTTCCAGCGCCCGTACGGCTCC |
| | | | TACGTGATGGAAAACGTGCTGTTCAAAATCTCCTTCCCGG |
| | | | CGGAGTTCCATTCGCAGACCGCCGTTGAAGCAGCGATGAC |
| | | | GCTGTATGAGCAGATGCAGGCGGCTGGAAAAACGGCGGC |
| | | | GGATATCGAAAAAGTAACGATTCGCACCCATGAAGCCTGT |
| | | | ATACGCATCATTGATAAAAAAGGCCCGCTGAATAATCCGG |
| | | | CTGACCGCGATCACTGTATTCAGTATATGGTGGCGATCCC |
| | | | ACTGCTGTTCGGACGCTTAACGGCGGCGGATTATGAGGAT |
| | | | GGCGTGGCGCAGGATAAACGTATTGACGCGCTGCGTGAAA |
| | | | AAACGCATTGCTTTGAAGACCCGGCGTTTACCACTGATTA |
| | | | TCATGACCCGGAAAAACGTTCGATTGCCAACGCCATTAGT |
| | | | CTTGAATTTACTGACGGTACCCGTTTTGACGAGGTGGTTGT |
| | | | CGAGTACCCGATCGGCCACGCGCGTCGTCGCGGCGACGGC |
| | | | ATTCCAAAACTTATCGAAAAATTTAAAATCAATCTGGCGC |
| | | | GCCAGTTCCCACCCCGCCAGCAACAACGCATCCTGGATGT |
| | | | CTCCCTGGACAGAACGCGCCTGGAGCAGATGCCGGTTAAT |
| | | | GAGTATCTCGACTTGTACGTCATCTAGAACCTGTCTCATTA |
| | | | GGCGTAAGTTCTACAGGAGAGCATTATGTCTTTTAGCGAA |
| | | | TTTTATCAGCGTTCGATTAACGAACCGGAGCAGTTCTGGG |
| | | | CTGAACAGGCCCGGCGTATCGACTGGCAGCAGCCGTTTAC |
| | | | GCAGACGCTGGACTACAGCAACCCGCCGTTTGCCCGCTGG |
| | | | TTTTGCGGCGGCACCACTAATCTGTGCCATAACGCGATTG |
| | | | ACCGCTGGCTGGATACCCAGCCGGATGCGCTGGCGCTGAT |
| | | | TGCGGTTTCCTCTGAGACCGAAGAAGAACGTACCTTCACC |
| | | | TTTCGTCAACTGTATGACGAGGTGAATGTCGTGGCCTCTAT |
| | | | GCTGCTGTCACTGGGCGTGCGGCGTGGCGATCGGGTACTG |
| | | | GTGTATATGCCGATGATTGCCGAGGCGCACATCACATTAC |
| | | | TGGCCTGCGCGCGCATTGGCGCGATCCATTCAGTGGTGTTT |
| | | | GGTGGTTTTGCCTCGCACAGTGTAGCCGCGCGCATCGACG |
| | | | ATGCCAGACCGGTGCTGATTGTCTCGGCGGACGCCGGAGC |
| | | | GCGAGGTGGGAAGGTCATTCCCTATAAAAAGCTTCTTGAT |
| | | | GAGGCGGTCGATCAGGCACAGCATCAGCCGAAGCATGTA |
| | | | CTGCTGGTGGATCGGGGGCTGGCGAAAATGGCGCGGGTTG |
| | | | CCGGGCGCGATGTGGATTTTGCGACCCTGCGCGAACACCA |
| | | | TGCCGGGGCGCGTGTGCCAGTGGCCTGGCTTGAATCTAAT |
| | | | GAAAGTTCCTGCATTCTTTATACCTCCGGCACTACCGGCAA |
| | | | ACCGAAAGGCGTTCAGCGTGACGTTGGTGGCTACGCCGTG |
| | | | GCGCTGGCGACATCGATGGACACCCTCTTTGGCGGCAAAG |
| | | | CGGGCGGCGTCTTTTTCTGCGCTTCGGATATCGGTTGGGTA |
| | | | GTGGGGCACTCTTATATTGTGTATGCGCCGCTGCTGGCGG |
| | | | GTATGGCGACCATCGTTTATGAAGGATTGCCGACGTATCC |
| | | | GGACTGCGGCGTATGGTGGAAAATTGTCGAGAAATATCGG |
| | | | GTGAGCCGGATGTTTTCAGCGCCAACCGCCATTCGTGTGC |
| | | | TGAAGAAATTTCCCACCGCGCAGATACGCAATCATGATCT |
| | | | CTCCTCGCTGGAAGTTCTCTATCTGGCAGGCGAGCCGCTC |
| | | | GACGAGCCAACGGCAGCCTGGGTTAGCGGAACACTGGGT |
| | | | GTGCCGGTGATCGACAATTACTGGCAGACCGAATCCGGCT |
| | | | GGCCGATTATGGCGCTGGCGCGCACGCTTGATGACAGACC |
| | | | ATCGCGTTTGGGCAGTCCCGGCGTGCCGATGTACGGCTAT |
| | | | AATGTTCAACTGCTCAACGAGGTGACCGGTGAACCCTGTG |
| | | | GTGCGAACGAAAAGGGAATGGTGGTTATTGAAGGGCCGC |
| | | | TGCCGCCGGGCTGCATTCAGACCATCTGGGGCGATGACGC |
| | | | ACGCTTTGTGAATACCTACTGGTCACTGTTTACTCGTCAGG |
| | | | TGTATGCCACCTTTGACTGGGGGATCCGCGACGCCGACGG |
| | | | CTATTATTTTATCCTTGGGCGCACGGATGATGTGATCAACG |
| | | | TCGCCGGACATCGTCTCGGCACCCGTGAGATAGAGGAGAG |
| | | | CATCTCCAGCTATCCCAACGTTGCGGAAGTGGCGGTGGTA |
| | | | GGGGTAAAAGACGCGCTGAAAGGGCAGGTAGCGGTAGCC |
| | | | TTCGTGATCCCGAAACAGAGTGACAGTCTGGAAGACCGCG |
| | | | AAGTGGCGCATTCGGAAGAGAAGGCGATTATGGCGCTGGT |
| | | | CGATAGTCAGATCGGCAACTTTGGCCGCCCGGCGCACGTG |
| | | | TGGTTTGTCTCGCAGCTACCAAAAACCCGATCCGGGAAGA |
| | | | TGCTCAGACGAACGATCCAGGCGATCTGCGAGGGCCGGG |
| | | | ATCCAGGCGATCTGACGACCATTGACGATCCGACGTCGTT |
| | | | GCAACAAATTCGCCAGGTCATTGAGGAGTAA |
| 95 | PccB | Bifidobacterium | MTDIMDSQAVKAAAAASAANAAQPSAHQPLRTAVVKAAEL |
| | (polypeptide) | longum | ARAAEERARDKQHAKGKKTARERLDLLFDTGTFEEIGRFQG |
| | | | GNIAGGNAGAAVITGFGQVYGRKVAVYAQDFSVKGGTLGT |
| | | | AEGEKICRLMDMAIDLKVPIVAIVDSGGARIQEGVAALTQYG |
| | | | RIFRKTCEASGFVPQLSLILGPCAGGAVYCPALTDLIIMTRENS |
| | | | NMFVTGPDVVKASTGETISMADLGGGEVHNRVSGVAHYLG |
| | | | EDESDAIDYARTVLAYLPSNSESKPPVYAYAVTRAERETAKR |
| | | | LATIVPTNERQPYDMLEVIRCIVDYGEFVQVQELFGASALVG |
| | | | FACIDGKPVGIVANQPNVLAGILDVDSSEKVARFVRLCDAFN |
| | | | LPVVTLVDVPGYKPGSDQEHAGIIRRGAKVIYAYANAQVPM |
| | | | VTVVLRKAFGGAYIVMGSKAIGADLNFAWPSSQIAVLGAAG |
| | | | AVNIIHRHDLAKAKASGQDVDALRAKYIKEYETSTVNANLSL |
| | | | EIGQIDGMIDPEQTREVIVESLATLATKRRVKRTTKHHGNQPL |
| 96 | pccB | Bifidobacterium | TCAGAGGGGCTGGTTGCCGTGGTGTTTGGTGGTGCGCTTG |
| | | longum | ACGCGCCGCTTGGTGGCGAGCGTGGCCAGCGATTCGACAA |
| | | | TCACCTCACGGGTCTGTTCGGGGTCGATCATGCCGTCGATC |
| | | | TGCCCGATTTCCAGTGACAGGTTCGCGTTGACGGTGCTGG |
| | | | TCTCGTACTCCTTGATGTACTTGGCCCGCAGCGCATCGACG |
| | | | TCCTGTCCGGAGGCCTTGGCCTTGGCCAGGTCGTGGCGGT |
| | | | GGATGATGTTCACCGCGCCGGCCGCGCCGAGCACCGCGAT |
| | | | CTGGGAGGAGGGCCACGCGAAGTTCAGGTCCGCGCCAAT |
| | | | GGCCTTGGATCCCATCACGATGTACGCGCCGCCGAACGCC |
| | | | TTGCGCAACACCACGGTCACCATCGGTACCTGTGCGTTGG |
| | | | CGTAGGCGTAGATCACCTTGGCGCCGCGGCGGATGATGCC |
| | | | GGCGTGTTCCTGGTCGGAGCCGGGCTTGTAGCCGGGCACA |
| | | | TCCACGAGGGTGACCACGGGCAGGTTGAACGCGTCGCACA |
| | | | GGCGTACGAATCGGGCGACTTTCTCGGACGAGTCGACGTC |
| | | | CAGGATGCCGGCGAGCACGTTCGGCTGGTTCGCCACGATG |
| | | | CCAACCGGCTTGCCGTCGATGCAGGCGAAGCCGACGAGCG |
| | | | CGGAGGCGCCGAACAGTTCCTGCACCTGCACGAATTCGCC |
| | | | GTAATCGACGATGCAACGAATCACTTCGAGCATGTCGTAA |
| | | | GGCTGACGTTCGTTGGTGGGCACGATGGTGGCAAGTCGCT |
| | | | TGGCGGTCTCGCGTTCGGCGCGGGTGACGGCGTATGCGTA |
| | | | GACCGGCGGCTTGCTTTCGCTGTTGGACGGCAGGTAGGCG |
| | | | AGCACGGTGCGCGCATAGTCGATGGCGTCGGATTCGTCCT |
| | | | CGCCGAGGTAGTGGGCCACGCCGGACACCCGGTTGTGCAC |
| | | | TTCGCCGCCGCCGAGGTCGGCCATGGAGATGGTCTCGCCG |
| | | | GTCGAGGCCTTGACCACGTCCGGTCCGGTGACGAACATGT |
| | | | TCGAGTTCTCACGGGTCATGATGATGAGGTCCGTCAGGGC |
| | | | CGGGCAGTAGACGGCACCGCCGGCGCAGGGGCCGAGAAT |
| | | | CAGGCTCAGCTGGGGCACGAAGCCGCTGGCCTCGCAAGTC |
| | | | TTGCGGAAGATGCGACCGTACTGGGTCAGGGCGGCCACGC |
| | | | CCTCCTGGATGCGGGCGCCGCCGGAGTCCACGATGGCCAC |
| | | | GATCGGCACTTTGAGGTCGATGGCCATGTCCATCAGTCGG |
| | | | CAGATCTTCTCGCCTTCGGCGGTGCCGAGGGTGCCGCCCTT |
| | | | GACGGAGAAGTCCTGGGCGTAGACGGCCACTTTGCGGCCG |
| | | | TAGACCTGGCCGAAGCCGGTGATGACGGCCGCACCGGCGT |
| | | | TGCCGCCGGCGATATTGCCGCCCTGGAAGCGGCCGATCTC |
| | | | CTCGAACGTGCCGGTGTCGAAGAGCAGGTCGAGGCGTTCG |
| | | | CGCGCGGTTTTCTTGCCTTTGGCGTGCTGCTTGTCGCGGGC |
| | | | GCGCTCTTCGGCGGCGCGGGCCAGTTCGGCGGCCTTGACC |
| | | | ACAGCGGTGCGCAGCGGCTGGTGGGCCGAAGGCTGGGCG |
| | | | GCGTTGGCGGCCGAGGCCGCAGCCGCGGCCTTCACGGCCT |
| | | | GCGAATCCATGATGTCAGTCAT |
| 97 | GltA | E. coli | MADTKAKLTLNGDTAVELDVLKGTLGQDVIDIRTLGSKGVF |
| | (polypeptide) | | TFDPGFTSTASCESKITFIDGDEGILLHRGFPIDQLATDSNYLE |
| | | | VCYILLNGEKPTQEQYDEFKTTVTRHTMIHEQITRLFHAFRRD |
| | | | SHPMAVMCGITGALAAFYHDSLDVNNPRHREIAAFRLLSKM |
| | | | PTMAAMCYKYSIGQPFVYPRNDLSYAGNFLNMMFSTPCEPY |
| | | | EVNPILERAMDRILILHADHEQNASTSTVRTAGSSGANPFACI |
| | | | AAGIASLWGPAHGGANEAALKMLEEISSVKHIPEFVRRAKDK |
| | | | NDSFRLMGFGHRVYKNYDPRATVMRETCHEVLKELGTKDD |
| | | | LLEVAMELENIALNDPYFIEKKLYPNVDFYSGIILKAMGIPSS |
| | | | MFTVIFAMARTVGWIAHWSEMHSDGMKIARPRQLYTGYEK |
| | | | RDFKSDIKR |
| 98 | gltA | E. coli | ATGGCTGATACAAAAGCAAAACTCACCCTCAACGGGGATA |
| | | | CAGCTGTTGAACTGGATGTGCTGAAAGGCACGCTGGGTCA |
| | | | AGATGTTATTGATATCCGTACTCTCGGTTCAAAAGGTGTGT |
| | | | TCACCTTTGACCCAGGCTTCACTTCAACCGCATCCTGCGAA |
| | | | TCTAAAATTACTTTTATTGATGGTGATGAAGGTATTTTGCT |
| | | | GCACCGCGGTTTCCCGATCGATCAGCTGGCGACCGATTCT |
| | | | AACTACCTGGAAGTTTGTTACATCCTGCTGAATGGTGAAA |
| | | | AACCGACTCAGGAACAGTATGACGAATTTAAAACTACGGT |
| | | | GACCCGTCATACCATGATCCACGAGCAGATTACCCGTCTG |
| | | | TTCCATGCTTTCCGTCGCGACTCGCATCCAATGGCAGTCAT |
| | | | GTGTGGTATTACCGGCGCGCTGGCGGCGTTCTATCACGAC |
| | | | TCGCTGGATGTTAACAATCCTCGTCACCGTGAAATTGCCG |
| | | | CGTTCCGCCTGCTGTCGAAAATGCCGACCATGGCCGCGAT |
| | | | GTGTTACAAGTATTCCATTGGTCAGCCATTTGTTTACCCGC |
| | | | GCAACGATCTCTCCTACGCCGGTAACTTCCTGAATATGAT |
| | | | GTTCTCCACGCCGTGCGAACCGTATGAAGTTAATCCGATT |
| | | | CTGGAACGTGCTATGGACCGTATTCTGATCCTGCACGCTG |
| | | | ACCATGAACAGAACGCCTCTACCTCCACCGTGCGTACCGC |
| | | | TGGCTCTTCGGGTGCGAACCCGTTTGCCTGTATCGCAGCA |
| | | | GGTATTGCTTCACTGTGGGGACCTGCGCACGGCGGTGCTA |
| | | | ACGAAGCGGCGCTGAAAATGCTGGAAGAAATCAGCTCCG |
| | | | TTAAACACATTCCGGAATTTGTTCGTCGTGCGAAAGACAA |
| | | | AAATGATTCTTTCCGCCTGATGGGCTTCGGTCACCGCGTGT |
| | | | ACAAAAATTACGACCCGCGCGCCACCGTAATGCGTGAAAC |
| | | | CTGCCATGAAGTGCTGAAAGAGCTGGGCACGAAGGATGA |
| | | | CCTGCTGGAAGTGGCTATGGAGCTGGAAAACATCGCGCTG |
| | | | AACGACCCGTACTTTATCGAGAAGAAACTGTACCCGAACG |
| | | | TCGATTTCTACTCTGGTATCATCCTGAAAGCGATGGGTATT |
| | | | CCGTCTTCCATGTTCACCGTCATTTTCGCAATGGCACGTAC |
| | | | CGTTGGCTGGATCGCCCACTGGAGCGAAATGCACAGTGAC |
| | | | GGTATGAAGATTGCCCGTCCGCGTCAGCTGTATACAGGAT |
| | | | ATGAAAAACGCGACTTTAAAAGCGATATCAAGCGTTAA |
| 99 | PhaA | Acinetobacter | MKDVVIVAAKRTAIGSFLGSLASLSAPQLGQTAIRAVLDSAN |
| | (polypeptide) | sp. | VKPEQVDQVIMGNVLTTGVGQNPARQAAIAAGIPVQVPAST |
| | | | LNVVCGSGLRAVHLAAQAIQCDEADIVVAGGQESMSQSAHY |
| | | | MQLRNGQKMGNAQLVDSMVADGLTDAYNQYQMGITAENI |
| | | | VEKLGLNREEQDQLALTSQQRAAAAQAAGKFKDEIAVVSIP |
| | | | QRKGEPVVFAEDEYIKANTSLESLTKLRPAFKKDGSVTAGNA |
| | | | SGINDGAAAVLMMSADKAAELGLKPLARIKGYAMSGIEPEI |
| | | | MGLGPVDAVKKTLNKAGWSLDQVDLIEANEAFAAQALGVA |
| | | | KELGLDLDKVNVNGGAIALGHPIGASGCRILVTLLHEMQRRD |
| | | | AKKGIATLCVGGGMGVALAVERD |
| 100 | PhaB | Acinetobacter | MSEQKVALVTGALGGIGSEICRQLVTAGYKIIATVVPREEDR |
| | (polypeptide) | sp. | EKQWLQSEGFQDSDVRFVLTDLNNHEAATAAIQEAIAAEGR |
| | | | VDVLVNNAGITRDATFKKMSYEQWSQVIDTNLKTLFTVTQP |
| | | | VFNKMLEQKSGRIVNISSVNGLKGQFGQANYSASKAGIIGFT |
| | | | KALAQEGARSNICVNVVAPGYTATPMVTAMREDVIKSIEAQI |
| | | | PLQRLAAPAEIAAAVMYLVSEHGAYVTGETLSINGGLYMH |
| 101 | PhaC | Acinetobacter | MNPNSFQFKENILQFFSVHDDIWKKLQEFYYGQSPINEALAQ |
| | (polypeptide) | sp. | LNKEDMSLFFEALSKNPARMMEMQWSWWQGQIQIYQNVL |
| | | | MRSVAKDVAPFIQPESGDRRFNSPLWQEHPNFDLLSQSYLLF |
| | | | SQLVQNMVDVVEGVPDKVRYRIHFFTRQMINALSPSNFLWT |
| | | | NPEVIQQTVAEQGENLVRGMQVFHDDVMNSGKYLSIRMVN |
| | | | SDSFSLGKDLAYTPGAVVFENDIFQLLQYEATTENVYQTPILV |
| | | | VPPFINKYYVLDLREQNSLVNWLRQQGHTVFLMSWRNPNAE |
| | | | QKELTFADLITQGSVEALRVIEEITGEKEANCIGYCIGGTLLAA |
| | | | TQAYYVAKRLKNHVKSATYMATIIDFENPGSLGVFINEPVVS |
| | | | GLENLNNQLGYFDGRQLAVTFSLLRENTLYWNYYIDNYLKG |
| | | | KEPSDFDILYWNSDGTNIPAKIHNFLLRNLYLNNELISPNAVK |
| | | | VNGVGLNLSRVKTPSFFIATQEDHIALWDTCFRGADYLGGES |
| | | | TLVLGESGHVAGIVNPPSRNKYGCYTNAAKFENTKQWLDGA |
| | | | EYHPESWWLRWQAWVTPYTGEQVPARNLGNAQYPSIEAAP |
| | | | GRYVLVNLF |
| 102 | phaABC | Acinetobacter | AAGCTTATAGCTAACACCGCAATCAATTTTTTCACTCGTCT |
| | | sp. | AGCGTCTGTCAAGCGCGTATTTTCAAGATTAAACCCGCGT |
| | | | CCTTTGAGACAACTGAATAAGGTTTCAATTTCCCAGCGTA |
| | | | ATGCATAATCCTGAATAGCATTGGCATTAAACTGAGGAGA |
| | | | AACGACGAGTAAAAGCTCTCCATTTTCTAACTGTAGTGCA |
| | | | CTTATATATAGTTTCACCCGACCAACCAAAATCCGTCGTTT |
| | | | ACGACATTCAATTTGACCAACTTTAAGATGGCGAAATAAA |
| | | | TCACTAATTTTATGATTCTTTCCTAAATGATTGGTGACAAT |
| | | | GAAGTTTTTTTAACACGAATGCAGAAGTTGATGTCTTGGTT |
| | | | CAATTAACCATGTAAACCACTGCTCACCGATAAACTCTCT |
| | | | GTCTGCGAACACATTCACAATACGGTCTTTACCAAAAATG |
| | | | GCTATAAAGCGTTGAATCAAAGCAATACGCTCTTTCGTAT |
| | | | CTGAATTTCCACGTTTATTAAGCAATGTCCAAAGGATAGG |
| | | | TATCGCTATTCCACGATAAACGATTGCGAGCATCAGGATA |
| | | | TTAATATTTCGTTTTCCCCATTTCCAATTGGTTCTATCTAAA |
| | | | GTCAGTTGCACTTGGTCGAATGAAAACATATTGAAAATCA |
| | | | ACTGAGAAATTTGACGATAATCAAAATACTGACCTGCAAA |
| | | | GAAGCGCTGCATACGTCGATAAAATGATTGTGGTAAGCAC |
| | | | TTGATGGGCAAGGCTTTAGATGCAGAAGAAAGATTACATG |
| | | | TTTGCTTTAAAATAATCACAAGCATGATGAGCGCAAAGCA |
| | | | CTTTAAATGTGACTTGTTCCATTTTAGATATTTGTTTAAGA |
| | | | TAAGATATAACTCATTGAGATGTGTCATAGTATTCGTCGTT |
| | | | AGAAAACAATTATTATGACATTATTTCAATGAGTTATCTAT |
| | | | TTTTGTCGTGTACAGAGCAATATTTGTTTACTTTTGACTTTA |
| | | | AAGCATCATCAAACTGCGATCTGTTTGCAATATAAAACGC |
| | | | TTAATTTCTAAACAAGAATAAAGAGGAAAAACTTCTTATT |
| | | | TTTTTATAACCTTATTCTGCTTAGGAAAACAACATGTCTGA |
| | | | ACAAAAAGTAGCTTTAGTGACGGGTGCATTAGGTGGTATT |
| | | | GGCAGTGAGATTTGCCGTCAACTGGTTACAGCAGGCTATA |
| | | | AAATTATCGCAACGGTTGTACCACGTGAAGAAGACCGCGA |
| | | | AAAACAATGGCTTCAAAGTGAAGGATTTCAAGACAGCGAT |
| | | | GTACGCTTTGTCTTAACGGATTTAAATAACCACGAAGCGG |
| | | | CAACAGCAGCTATTCAAGAAGCAATTGCTGCTGAAGGTCG |
| | | | TGTCGATGTGCTGGTCAATAATGCAGGCATCACCCGTGAT |
| | | | GCAACTTTTAAAAAGATGAGCTATGAACAGTGGTCACAAG |
| | | | TCATTGATACCAACTTAAAAACATTGTTCACAGTGACTCA |
| | | | GCCCGTGTTTAACAAAATGTTAGAACAAAAGTCGGGACGT |
| | | | ATTGTCAATATCAGCTCAGTCAACGGTTTAAAAGGTCAGT |
| | | | TTGGACAAGCCAACTACTCTGCGAGCAAAGCCGGCATTAT |
| | | | CGGTTTTACCAAAGCCTTAGCTCAAGAAGGTGCACGTTCA |
| | | | AATATTTGTGTAAACGTGGTTGCGCCTGGCTATACCGCAA |
| | | | CACCAATGGTTACTGCCATGCGTGAAGATGTGATTAAAAG |
| | | | CATTGAAGCACAAATTCCTCTACAACGTCTTGCTGCGCCA |
| | | | GCTGAAATTGCCGCTGCTGTTATGTACTTGGTCAGCGAGC |
| | | | ACGGTGCGTACGTGACAGGCGAAACCTTATCGATTAATGG |
| | | | CGGTTTATACATGCACTAAACCGTGCAGCCCCTATTTTCAT |
| | | | TTACAAGTTTATTTACTGGAGTTACACCATGCTATACGGCG |
| | | | ACTTATTTTCAAATATGAATGCACAATACAAAAACGTATTT |
| | | | GAACCGTACACAAAATTCAACAGCTTAGTGGCTAAAAACT |
| | | | TTGCTGACTTAACCAACCTACAATTAGAAGCAGCACGCAA |
| | | | CTATGCCAACATTGGTCTAGCGCAAATGTTTGCCAATAGT |
| | | | GAAGTTAAAGACATGCAAAGCATGGTGAATTGCACCACCA |
| | | | AGCAATTAGAAACCATGAACAAACTTAGTCAGCAAATGAT |
| | | | TGAAGATGGCAAAAAGTTGGCAACACTAACGACTGAATTC |
| | | | AAATCGGAATTTGAAAAGTTAGTTAGCGAATCTATGCCTA |
| | | | ACAATAAATAACACTGCTCTGAAAACCATGCGTTATCAGG |
| | | | ACGAATGTTACGGGGAAGTGTGAAAATTTCCCCGTTTTAG |
| | | | TTTCAGCCCTGCACTCAATTTGATTGCTAAAAGCCATGTGC |
| | | | TATGGAGCGATGAAATGAACCCGAACTCATTTCAATTCAA |
| | | | AGAAAACATACTACAATTTTTTTCTGTACATGATGACATCT |
| | | | GGAAAAAATTACAAGAATTTTATTATGGGCAAAGCCCAAT |
| | | | TAATGAGGCTTTGGCGCAGCTCAACAAAGAAGATATGTCT |
| | | | TTGTTCTTTGAAGCACTATCTAAAAACCCAGCTCGCATGAT |
| | | | GGAAATGCAATGGAGCTGGTGGCAAGGTCAAATACAAAT |
| | | | CTACCAAAATGTGTTGATGCGCAGCGTGGCCAAAGATGTA |
| | | | GCACCATTTATTCAGCCTGAAAGTGGTGATCGTCGTTTTAA |
| | | | CAGCCCATTATGGCAAGAACACCCAAATTTTGACTTGTTG |
| | | | TCACAGTCTTATTTACTGTTTAGCCAGTTAGTGCAAAACAT |
| | | | GGTAGATGTGGTCGAAGGTGTTCCAGACAAAGTTCGCTAT |
| | | | CGTATTCACTTCTTTACCCGCCAAATGATCAATGCGTTATC |
| | | | TCCAAGTAACTTTCTGTGGACTAACCCAGAAGTGATTCAG |
| | | | CAAACTGTAGCTGAACAAGGTGAAAACTTAGTCCGTGGCA |
| | | | TGCAAGTTTTCCATGATGATGTCATGAATAGCGGCAAGTA |
| | | | TTTATCTATTCGCATGGTGAATAGCGACTCTTTCAGCTTGG |
| | | | GCAAAGATTTAGCTTACACCCCTGGTGCAGTCGTCTTTGA |
| | | | AAATGACATTTTCCAATTATTGCAATATGAAGCAACTACT |
| | | | GAAAATGTGTATCAAACCCCTATTCTAGTCGTACCACCGTT |
| | | | TATCAATAAATATTATGTGCTGGATTTACGCGAACAAAAC |
| | | | TCTTTAGTGAACTGGTTGCGCCAGCAAGGTCATACAGTCTT |
| | | | TTTAATGTCATGGCGTAACCCAAATGCCGAACAGAAAGAA |
| | | | TTGACTTTTGCCGATCTCATTACACAAGGTTCAGTGGAAGC |
| | | | TTTGCGTGTAATTGAAGAAATTACCGGTGAAAAAGAGGCC |
| | | | AACTGCATTGGCTACTGTATTGGTGGTACGTTACTTGCTGC |
| | | | GACTCAAGCCTATTACGTGGCAAAACGCCTGAAAAATCAC |
| | | | GTAAAGTCTGCGACCTATATGGCCACCATTATCGACTTTG |
| | | | AAAACCCAGGCAGCTTAGGTGTATTTATTAATGAACCTGT |
| | | | AGTGAGCGGTTTAGAAAACCTGAACAATCAATTGGGTTAT |
| | | | TTCGATGGTCGTCAGTTGGCAGTTACCTTCAGTTTACTGCG |
| | | | TGAAAATACGCTGTACTGGAATTACTACATCGACAACTAC |
| | | | TTAAAAGGTAAAGAACCTTCTGATTTTGATATTTTATATTG |
| | | | GAACAGCGATGGTACGAATATCCCTGCCAAAATTCATAAT |
| | | | TTCTTATTGCGCAATTTGTATTTGAACAATGAATTGATTTC |
| | | | ACCAAATGCCGTTAAGGTTAACGGTGTGGGCTTGAATCTA |
| | | | TCTCGTGTAAAAACACCAAGCTTCTTTATTGCGACGCAGG |
| | | | AAGACCATATCGCACTTTGGGATACTTGTTTCCGTGGCGC |
| | | | AGATTACTTGGGTGGTGAATCAACCTTGGTTTTAGGTGAAT |
| | | | CTGGACACGTAGCAGGTATTGTCAATCCTCCAAGCCGTAA |
| | | | TAAATACGGTTGCTACACCAATGCTGCCAAGTTTGAAAAT |
| | | | ACCAAACAATGGCTAGATGGCGCAGAATATCACCCTGAAT |
| | | | CTTGGTGGTTGCGCTGGCAGGCATGGGTCACACCGTACAC |
| | | | TGGTGAACAAGTCCCTGCCCGCAACTTGGGTAATGCGCAG |
| | | | TATCCAAGCATTGAAGCGGCACCGGGTCGCTATGTTTTGG |
| | | | TAAATTTATTCTAATCGGTCATATAACAACAGCCATGCAG |
| | | | ATGCTATATATCATGTGCATCCACAGAAACATGAACACAA |
| | | | AATTTAAGGATATAAAATGAAAGATGTTGTGATTGTTGCA |
| | | | GCAAAACGTACTGCGATTGGTAGCTTTTTAGGTAGTCTTGC |
| | | | ATCTTTATCTGCACCACAGTTGGGGCAAACAGCAATTCGT |
| | | | GCAGTTTTAGACAGCGCTAATGTAAAACCTGAACAAGTTG |
| | | | ATCAGGTGATTATGGGCAACGTACTCACGACAGGCGTGGG |
| | | | ACAAAACCCTGCACGTCAGGCAGCAATTGCTGCTGGTATT |
| | | | CCAGTACAAGTGCCTGCATCTACGCTGAATGTCGTCTGTG |
| | | | GTTCAGGTTTGCGTGCGGTACATTTGGCAGCACAAGCCAT |
| | | | TCAATGCGATGAAGCCGACATTGTGGTCGCAGGTGGTCAA |
| | | | GAATCTATGTCACAAAGTGCGCACTATATGCAGCTGCGTA |
| | | | ATGGGCAAAAAATGGGTAATGCACAATTGGTGGATAGCAT |
| | | | GGTGGCTGATGGTTTAACCGATGCCTATAACCAGTATCAA |
| | | | ATGGGTATTACCGCAGAAAATATTGTAGAAAAACTGGGTT |
| | | | TAAACCGTGAAGAACAAGATCAACTTGCATTGACTTCACA |
| | | | ACAACGTGCTGCGGCAGCTCAGGCAGCTGGCAAGTTTAAA |
| | | | GATGAAATTGCCGTAGTCAGCATTCCACAACGTAAAGGTG |
| | | | AGCCTGTTGTATTTGCTGAAGATGAATACATTAAAGCCAA |
| | | | TACCAGCCTTGAAAGCCTCACAAAACTACGCCCAGCCTTT |
| | | | AAAAAAGATGGTAGCGTAACCGCAGGTAATGCTTCAGGC |
| | | | ATTAATGATGGTGCAGCAGCAGTACTGATGATGAGTGCGG |
| | | | ACAAAGCAGCAGAATTAGGTCTTAAGCCATTGGCACGTAT |
| | | | TAAAGGCTATGCCATGTCTGGTATTGAGCCTGAAATTATG |
| | | | GGGCTTGGTCCTGTCGATGCAGTAAAGAAAACCCTCAACA |
| | | | AAGCAGGCTGGAGCTTAGATCAGGTTGATTTGATTGAAGC |
| | | | CAATGAAGCATTTGCTGCACAGGCTTTGGGTGTTGCTAAA |
| | | | GAATTAGGCTTAGACCTGGATAAAGTCAACGTCAATGGCG |
| | | | GTGCAATTGCATTGGGTCACCCAATTGGGGCTTCAGGTTG |
| | | | CCGTATTTTGGTGACTTTATTACATGAAATGCAGCGCCGTG |
| | | | ATGCCAAGAAAGGCATTGCAACCCTCTGTGTTGGCGGTGG |
| | | | TATGGGTGTTGCACTTGCAGTTGAACGTGACTAAGTACAC |
| | | | CATTGCATCGAATCTTGAAACTTGATAAAGATTGACAATA |
| | | | AATTCAATACATAATGGGAGCTCAGGCTTCCATTATTTCTA |
| | | | GCTGAGCGCATTTCTAATATTAAGGCTTCTAGCTCAGCATT |
| | | | GATTTTAGTATTTGGCGATTTTAAGGGACGTCTACTCTGAC |
| | | | TACTTAATCCATCAATACCTTGCTCAGAATATCGTTTCCAC |
| | | | CACTTGCGTAACGTTGGTCTAGA |
| 103 | AccA | E. coli | MSLNFLDFEQPIAELEAKIDSLTAVSRQDEKLDINIDEEVHRL |
| | (polypeptide) | | REKSVELTRKIFADLGAWQIAQLARHPQRPYTLDYVRLAFDE |
| | | | FDELAGDRAYADDKAIVGGIARLDGRPVMIIGHQKGRETKE |
| | | | KIRRNFGMPAPEGYRKALRLMQMAERFKMPIITFIDTPGAYP |
| | | | GVGAEERGQSEAIARNLREMSRLGVPVVCTVIGEGGSGGAL |
| | | | AIGVGDKVNMLQYSTYSVISPEGCASILWKSADKAPLAAEA |
| | | | MGIIAPRLKELKLIDSIIPEPLGGAHRNPEAMAASLKAQLLAD |
| | | | LADLDVLSTEDLKNRRYQRLMSYGYA |
| 104 | accA | E. coli | ATGAGTCTGAATTTCCTTGATTTTGAACAGCCGATTGCAGA |
| | | | GCTGGAAGCGAAAATCGATTCTCTGACTGCGGTTAGCCGT |
| | | | CAGGATGAGAAACTGGATATTAACATCGATGAAGAAGTG |
| | | | CATCGTCTGCGTGAAAAAAGCGTAGAACTGACACGTAAAA |
| | | | TCTTCGCCGATCTCGGTGCATGGCAGATTGCGCAACTGGC |
| | | | ACGCCATCCACAGCGTCCTTATACCCTGGATTACGTTCGCC |
| | | | TGGCATTTGATGAATTTGACGAACTGGCTGGCGACCGCGC |
| | | | GTATGCAGACGATAAAGCTATCGTCGGTGGTATCGCCCGT |
| | | | CTCGATGGTCGTCCGGTGATGATCATTGGTCATCAAAAAG |
| | | | GTCGTGAAACCAAAGAAAAAATTCGCCGTAACTTTGGTAT |
| | | | GCCAGCGCCAGAAGGTTACCGCAAAGCACTGCGTCTGATG |
| | | | CAAATGGCTGAACGCTTTAAGATGCCTATCATCACCTTTAT |
| | | | CGACACCCCGGGGGCTTATCCTGGCGTGGGCGCAGAAGAG |
| | | | CGTGGTCAGTCTGAAGCCATTGCACGCAACCTGCGTGAAA |
| | | | TGTCTCGCCTCGGCGTACCGGTAGTTTGTACGGTTATCGGT |
| | | | GAAGGTGGTTCTGGCGGTGCGCTGGCGATTGGCGTGGGCG |
| | | | ATAAAGTGAATATGCTGCAATACAGCACCTATTCCGTTAT |
| | | | CTCGCCGGAAGGTTGTGCGTCCATTCTGTGGAAGAGCGCC |
| | | | GACAAAGCGCCGCTGGCGGCTGAAGCGATGGGTATCATTG |
| | | | CTCCGCGTCTGAAAGAACTGAAACTGATCGACTCCATCAT |
| | | | CCCGGAACCACTGGGTGGTGCTCACCGTAACCCGGAAGCG |
| | | | ATGGCGGCATCGTTGAAAGCGCAACTGCTGGCGGATCTGG |
| | | | CCGATCTCGACGTGTTAAGCACTGAAGATTTAAAAAATCG |
| | | | TCGTTATCAGCGCCTGATGAGCTACGGTTACGCGTAA |
| 105 | MmcE | Pelotomaculum | MFKQDQLDKIAAKKESWSAKLAAAVKKRPEREAQFMTDSGI |
| | (polypeptide) | thermopropionicum | EVNTVYTPLDIADMDYERDLGLPGEYPYTRGVQPNMYRGRL |
| | | | WTMRQYAGFGTAEETNQRFRYLLEQGQTGLSCAFDLPTQIG |
| | | | YDSDHPMARGEIGKVGVAIDSLQDMETLFDQIPLGKVSTSMT |
| | | | INAPAGILLAMYIVVAEKQGFKRAELNGTIQNDIIKEYVGRGT |
| | | | YILPPEPSMRLITNIFEFCSKEVPNWNTISISGYHIREAGCTAAQ |
| | | | EIAFTLADGIAYVDAAIKAGLDVDQFGPRLSFFFNAHLNFLEE |
| | | | IAKFRAARRVWAKIMKERFGAKDPRSWTLRFHTQTAGCSLT |
| | | | AQQPMVNIMRTAFEALAAVLGGTQSLHTNSYDEALALPSDE |
| | | | SVLIALRTQQVIGYEIGVCDVVDPLGGSYYIESLTNQLEAKA |
| | | | WEYIEKIDALGGAVKAIDYMQKEIHNAAYQYQLAIDNKKKT |
| | | | VIGVNKFQLKEEEKPKNLLKVDLSVGERQIAKLKKLKEERDN |
| | | | AKVEALLKQVREAAQSDANMMPVFIDAVKEYVTLGEICGVL |
| | | | RDVFGEYKQQIVF |
| 106 | mmcE | Pelotomaculum | TTGTTTAAACAGGATCAACTGGACAAAATTGCTGCCAAGA |
| | | thermopropionicum | AAGAAAGCTGGTCTGCAAAGCTGGCAGCAGCGGTCAAAA |
| | | | AGCGTCCGGAAAGAGAAGCTCAATTCATGACCGACTCTGG |
| | | | AATTGAAGTCAACACCGTTTACACTCCTCTTGATATTGCAG |
| | | | ACATGGATTATGAGCGTGACCTGGGCCTGCCTGGGGAATA |
| | | | CCCGTATACCCGGGGTGTGCAGCCTAACATGTACCGCGGC |
| | | | CGCCTCTGGACCATGCGCCAGTACGCAGGTTTTGGCACAG |
| | | | CCGAAGAAACCAACCAGCGTTTCCGCTATCTCCTGGAGCA |
| | | | AGGGCAGACAGGCCTTAGCTGCGCCTTCGATTTGCCTACT |
| | | | CAGATCGGCTACGATTCGGACCATCCTATGGCAAGGGGAG |
| | | | AAATCGGTAAGGTTGGCGTTGCTATAGACTCCCTGCAGGA |
| | | | CATGGAAACTCTTTTCGACCAGATCCCCCTGGGCAAGGTC |
| | | | AGCACTTCCATGACCATCAACGCCCCGGCAGGCATACTAC |
| | | | TGGCCATGTATATTGTGGTGGCTGAAAAACAGGGGTTTAA |
| | | | GAGGGCAGAATTAAACGGAACGATTCAAAACGATATTATT |
| | | | AAGGAATATGTCGGCCGGGGAACATACATCCTGCCGCCTG |
| | | | AGCCCTCAATGCGTTTAATTACAAATATTTTTGAGTTCTGT |
| | | | TCCAAAGAAGTGCCCAACTGGAATACGATCAGCATCAGCG |
| | | | GCTATCATATCCGTGAAGCGGGTTGCACCGCAGCTCAGGA |
| | | | AATAGCCTTTACCCTAGCGGACGGCATTGCCTATGTGGAT |
| | | | GCAGCCATTAAAGCAGGCCTGGATGTTGATCAGTTTGGTC |
| | | | CTCGCCTTTCATTCTTCTTCAATGCTCACCTGAACTTCCTCG |
| | | | AGGAAATTGCAAAATTCCGGGCGGCACGGCGCGTCTGGGC |
| | | | GAAGATTATGAAGGAACGTTTCGGAGCCAAAGATCCGCGC |
| | | | TCGTGGACCCTGCGCTTCCACACTCAGACTGCCGGCTGCA |
| | | | GCCTGACGGCCCAGCAGCCGATGGTAAATATCATGAGGAC |
| | | | CGCATTTGAGGCCCTGGCTGCCGTACTGGGCGGGACTCAG |
| | | | TCCCTGCACACCAACTCCTATGACGAAGCCCTGGCCCTTCC |
| | | | CAGCGACGAGTCGGTGCTTATTGCATTGCGCACACAGCAG |
| | | | GTGATCGGCTATGAAATCGGCGTTTGCGACGTGGTTGACC |
| | | | CGCTTGGCGGATCCTACTACATTGAAAGCCTGACCAACCA |
| | | | GCTTGAAGCAAAAGCCTGGGAGTACATTGAGAAGATTGAT |
| | | | GCCCTCGGCGGTGCCGTAAAGGCCATCGATTACATGCAGA |
| | | | AGGAGATCCACAACGCCGCTTACCAGTATCAACTGGCTAT |
| | | | TGACAATAAGAAGAAGACCGTTATCGGAGTGAACAAATTC |
| | | | CAGTTGAAGGAAGAAGAAAAGCCAAAGAACCTGCTGAAA |
| | | | GTGGACCTCTCCGTGGGCGAACGGCAGATTGCGAAGCTCA |
| | | | AAAAGCTTAAGGAAGAAAGAGATAACGCCAAGGTTGAAG |
| | | | CCCTGCTGAAACAAGTGCGCGAGGCGGCGCAGAGCGATG |
| | | | CAAACATGATGCCTGTCTTTATCGATGCGGTTAAGGAATA |
| | | | CGTTACTCTGGGCGAGATCTGCGGCGTCCTGAGAGACGTA |
| | | | TTCGGCGAATACAAGCAGCAAATCGTATTCTAG |
| 107 | Acs | E. coli | MSQIHKHTIPANIADRCLINPQQYEAMYQQSINVPDTFWGEQ |
| | (polypeptide) | | GKILDWIKPYQKVKNTSFAPGNVSIKWYEDGTLNLAANCLD |
| | | | RHLQENGDRTAIIWEGDDASQSKHISYKELHRDVCRFANTLL |
| | | | ELGIKKGDVVAIYMPMVPEAAVAMLACARIGAVHSVIFGGF |
| | | | SPEAVAGRIIDSNSRLVITSDEGVRAGRSIPLKKNVDDALKNP |
| | | | NVTSVEHVVVLKRTGGKIDWQEGRDLWWHDLVEQASDQH |
| | | | QAEEMNAEDPLFILYTSGSTGKPKGVLHTTGGYLVYAALTFK |
| | | | YVFDYHPGDIYWCTADVGWVTGHSYLLYGPLACGATTLMF |
| | | | EGVPNWPTPARMAQVVDKHQVNILYTAPTAIRALMAEGDK |
| | | | AIEGTDRSSLRILGSVGEPINPEAWEWYWKKIGNEKCPVVDT |
| | | | WWQTETGGFMITPLPGATELKAGSATRPFFGVQPALVDNEG |
| | | | NPLEGATEGSLVITDSWPGQARTLFGDHERFEQTYFSTFKNM |
| | | | YFSGDGARRDEDGYYWITGRVDDVLNVSGHRLGTAEIESAL |
| | | | VAHPKIAEAAVVGIPHNIKGQAIYAYVTLNHGEEPSPELYAE |
| | | | VRNWVRKEIGPLATPDVLHWTDSLPKTRSGKIMRRILRKIAA |
| | | | GDTSNLGDTSTLADPGVVEKLLEEKQAIAMPS |
| 108 | acs | E. coli | ATGAGCCAAATTCACAAACACACCATTCCTGCCAACATCG |
| | | | CAGACCGTTGCCTGATAAACCCTCAGCAGTACGAGGCGAT |
| | | | GTATCAACAATCTATTAACGTACCTGATACCTTCTGGGGC |
| | | | GAACAGGGAAAAATTCTTGACTGGATCAAACCTTACCAGA |
| | | | AGGTGAAAAACACCTCCTTTGCCCCCGGTAATGTGTCCAT |
| | | | TAAATGGTACGAGGACGGCACGCTGAATCTGGCGGCAAA |
| | | | CTGCCTTGACCGCCATCTGCAAGAAAACGGCGATCGTACC |
| | | | GCCATCATCTGGGAAGGCGACGACGCCAGCCAGAGCAAA |
| | | | CATATCAGCTATAAAGAGCTGCACCGCGACGTCTGCCGCT |
| | | | TCGCCAATACCCTGCTCGAGCTGGGCATTAAAAAAGGTGA |
| | | | TGTGGTGGCGATTTATATGCCGATGGTGCCGGAAGCCGCG |
| | | | GTTGCGATGCTGGCCTGCGCCCGCATTGGCGCGGTGCATT |
| | | | CGGTGATTTTCGGCGGCTTCTCGCCGGAAGCCGTTGCCGG |
| | | | GCGCATTATTGATTCCAACTCACGACTGGTGATCACTTCCG |
| | | | ACGAAGGTGTGCGTGCCGGGCGCAGTATTCCGCTGAAGAA |
| | | | AAACGTTGATGACGCGCTGAAAAACCCGAACGTCACCAGC |
| | | | GTAGAGCATGTGGTGGTACTGAAGCGTACTGGCGGGAAA |
| | | | ATTGACTGGCAGGAAGGGCGCGACCTGTGGTGGCACGACC |
| | | | TGGTTGAGCAAGCGAGCGATCAGCACCAGGCGGAAGAGA |
| | | | TGAACGCCGAAGATCCGCTGTTTATTCTCTACACCTCCGGT |
| | | | TCTACCGGTAAGCCAAAAGGTGTGCTGCATACTACCGGCG |
| | | | GTTATCTGGTGTACGCGGCGCTGACCTTTAAATATGTCTTT |
| | | | GATTATCATCCGGGTGATATCTACTGGTGCACCGCCGATG |
| | | | TGGGCTGGGTGACCGGACACAGTTACTTGCTGTACGGCCC |
| | | | GCTGGCCTGCGGTGCGACCACGCTGATGTTTGAAGGCGTA |
| | | | CCCAACTGGCCGACGCCTGCCCGTATGGCGCAGGTGGTGG |
| | | | ACAAGCATCAGGTCAATATTCTCTATACCGCACCCACGGC |
| | | | GATCCGCGCGCTGATGGCGGAAGGCGATAAAGCGATCGA |
| | | | AGGCACCGACCGTTCGTCGCTGCGCATTCTCGGTTCCGTG |
| | | | GGCGAGCCAATTAACCCGGAAGCGTGGGAGTGGTACTGG |
| | | | AAAAAAATCGGCAACGAGAAATGTCCGGTGGTCGATACCT |
| | | | GGTGGCAGACCGAAACCGGCGGTTTCATGATCACCCCGCT |
| | | | GCCTGGCGCTACCGAGCTGAAAGCCGGTTCGGCAACACGT |
| | | | CCGTTCTTCGGCGTGCAACCGGCGCTGGTCGATAACGAAG |
| | | | GTAACCCGCTGGAGGGGGCCACCGAAGGTAGCCTGGTAAT |
| | | | CACCGACTCCTGGCCGGGTCAGGCGCGTACGCTGTTTGGC |
| | | | GATCACGAACGTTTTGAACAGACCTACTTCTCCACCTTCAA |
| | | | AAATATGTATTTCAGCGGCGACGGCGCGCGTCGCGATGAA |
| | | | GATGGCTATTACTGGATAACCGGGCGTGTGGACGACGTGC |
| | | | TGAACGTCTCCGGTCACCGTCTGGGGACGGCAGAGATTGA |
| | | | GTCGGCGCTGGTGGCGCATCCGAAGATTGCCGAAGCCGCC |
| | | | GTAGTAGGTATTCCGCACAATATTAAAGGTCAGGCGATCT |
| | | | ACGCCTACGTCACGCTTAATCACGGGGAGGAACCGTCACC |
| | | | AGAACTGTACGCAGAAGTCCGCAACTGGGTGCGTAAAGA |
| | | | GATTGGCCCGCTGGCGACGCCAGACGTGCTGCACTGGACC |
| | | | GACTCCCTGCCTAAAACCCGCTCCGGCAAAATTATGCGCC |
| | | | GTATTCTGCGCAAAATTGCGGCGGGCGATACCAGCAACCT |
| | | | GGGCGATACCTCGACGCTTGCCGATCCTGGCGTAGTCGAG |
| | | | AAGCTGCTTGAAGAGAAGCAGGCTATCGCGATGCCATCGT |
| | | | AA |
| 109 | MutA | Propionibacterium | MSSTDQGTNPADTDDLTPTTLSLAGDFPKATEEQWEREVEK |
| | | freudenreichii | VLNRGRPPEKQLTFAECLKRLTVHTVDGIDIVPMYRPKDAPK |
| | | subsp. | KLGYPGVAPFTRGTTVRNGDMDAWDVRALHEDPDEKFTRK |
| | | shermanii | AILEGLERGVTSLLLRVDPDAIAPEHLDEVLSDVLLEMTKVE |
| | | | VFSRYDQGAAAEALVSVYERSDKPAKDLALNLGLDPIAFAA |
| | | | LQGTEPDLTVLGDWVRRLAKFSPDSRAVTIDANIYHNAGAG |
| | | | DVAELAWALATGAEYVRALVEQGFTATEAFDTINFRVTATH |
| | | | DQFLTIARLRALREAWARIGEVFGVDEDKRGARQNAITSWR |
| | | | DVTREDPYVNILRGSIATFSASVGGAESITTLPFTQALGLPEDD |
| | | | FPLRIARNTGIVLAEEVNIGRVNDPAGGSYYVESLTRSLADAA |
| | | | WKEFQEVEKLGGMSKAVMTEHVTKVLDACNAERAKRLAN |
| | | | RKQPITAVSEFPMIGARSIETKPFPAAPARKGLAWHRDSEVFE |
| | | | QLMDRSTSVSERPKVFLACLGTRRDFGGREGFSSPVWHIAGI |
| | | | DTPQVEGGTTAEIVEAFKKSGAQVADLCSSAKVYAQQGLEV |
| | | | AKALKAAGAKALYLSGAFKEFGDDAAEAEKLIDGRLFMGM |
| | | | DVVDTLSSTLDILGVAK |
| 110 | muta | Propionibacterium | TCAGGCCTCCAGCTTGTCCAGGGTGGAGGTCAGCAACTCC |
| | | freudenreichii | ACCACATTCATTCCGTCGAAGACGTTGCCGTCGATCACGG |
| | | subsp. | CGTTCACCTCGGCCTCGTCGCCGCCGAGTTCCTTCAGCTGC |
| | | shermanii | CCGGCGAGCCGCACCTCCTGGGCGCCCGCCTCCTTCAGGG |
| | | | CCTTCGCGACGGCGAGACCGTGGGCGGCGTAGACCTTCGC |
| | | | GCTGGAGCACAGGACCGCGATGTCGGTGCCCGCCTCCTGC |
| | | | ATGGCCTTGACGAACACCTCCGGGTTGGTGCCCTCCGCGA |
| | | | TCACGGTGTTGATGCCACCCACGTGGTACAGGTTCGAGGT |
| | | | GAAGCCCTCGCGACCACCGAAGTCGCGCCGGGTGCCGAG |
| | | | GCAGGCCAGCAGCACGGTCGGGGTCTTCTTGGCGGCCTTG |
| | | | GAACGGTCCCGGAGGTCCTCGAAGACCTGGCTGTCGCGCA |
| | | | CGAACGGGATGCCGCCGAGCTTCGGGGCCGCGGGGCGCG |
| | | | GGGCGCGTTCGAGGGCCTTCTCGAGGTGGTTCGGGAACAT |
| | | | CGAGACGCCCGTCAGCGGCAGCTTGCGGGTGGCGAGCAG |
| | | | CTTGGCGCGGGCCTCGTTGATTTCCTTGAGCTGGGCGGCC |
| | | | ACGGTGCCATCGGCGATGGCGGCAGCCATACCCTTCTCGT |
| | | | CGAGCTGACCGAACAGCTCCCAGGCCTTCTCGCAGAGCTG |
| | | | CTTGGTCATGGACTCGACGAACCATGCGCCGCCGGCCGGG |
| | | | TCGTTGACGCGGCCGATGTTCGACTCCTCGGCCAGCACCA |
| | | | CCTGGGTGTTGCGGGCGATACGGCGGGTCAGGACGTCGGG |
| | | | AAGACCGATCACGGTGTCGAGCGGCAACACGGTGATGAA |
| | | | CTCGGCCTGGCCGACGGCCGCGGCGAAGGCCGAGATGGT |
| | | | GCCGCGCAGCACGTTGACGTAGGCGTCGTCGCGGGTGATC |
| | | | TCGCGCAGCGACGTGACGGCGTGCTGCACCGCGCCGCGTT |
| | | | TCTCCGGGCTCACCCCGAGCACCTCGCCGACCCGGTTCCA |
| | | | CAGGGTGCGCAGCGCGCGCAGCCGGGAGATGGTGATGAA |
| | | | CTGGTTGGTGTTCGCGGAGACCCGGAACAGGATGCTGTCG |
| | | | AAGGCCTCGTCGGCGCTCAGCCCGAGATCGGTGAGGGCGC |
| | | | GCACGTACTCGATGCCCGTGGCCAGCGCGTAGGCCAGCTG |
| | | | GGCGACGTCACCGGCGCCCATGGAATCGTAACGGGAGGC |
| | | | GTCCACGACGATGGGACGCACGCCCGAGAACGGCTTTGCA |
| | | | AGCTCCACGGCCCTCGCGATCACCGAGAGGTCGGGGGTGG |
| | | | TTCCGTTGAGGGCGGCGAAACCGATCGGGTCGATGCCGAG |
| | | | GCTGCCGCGGATGTTCTCCTTACCGGAGGCGGCGAAAGCC |
| | | | GCGGCCAGGGCCTCGGCGGCCGCCAGCTCATCGGTGTTGG |
| | | | AGGAAACATGTGTGGGGGCAAGGTCGAACAGGACATCGC |
| | | | TCAGGACCTCAGCCAGCTTGTCTGCGGGGACCGCATCGGG |
| | | | ATCCACGCGCACCCAGACCGCGGAGGTGCCGCGCTCCAGG |
| | | | TCGGTGTCCACGGCCTTGCGGGCCTCGGCCGGGTCGGGTT |
| | | | CCTCAATGAGCTGAGCACTGAACCAGCCCTCATCCATCTC |
| | | | TCCTGCACGGACCGTGGTGCCGCGCGTGAATGGGGCCACT |
| | | | CCGGGGAAACCAAGCTCCTTGACGCCATCGTCAATGGTGT |
| | | | ACAGCGGCTTGATCACAAGCCCATCGACGGTGTGGCTCGT |
| | | | CAGGCGCTTGTATGCCTGCTCAATGTTGAGTTCCTTGCCCT |
| | | | CGGGACGCCTCCGGTTCAGTACCTTCAGCACCTCTTTCTCC |
| | | | CAGTCTGCAAGGCTGGGAGTGGCGAAGTCAGCGGCGAGA |
| | | | CTGATCTCGGCCGCGCTCGTTGATTCTGCGCTCAT |
| 111 | MutB | Propionibacterium | MSTLPRFDSVDLGNAPVPADAARRFEELAAKAGTGEAWETA |
| | | freudenreichii | EQIPVGTLFNEDVYKDMDWLDTYAGIPPFVHGPYATMYAFR |
| | | subsp. | PWTIRQYAGFSTAKESNAFYRRNLAAGQKGLSVAFDLPTHR |
| | | shermanii | GYDSDNPRVAGDVGMAGVAIDSIYDMRELFAGIPLDQMSVS |
| | | | MTMNGAVLPILALYVVTAEEQGVKPEQLAGTIQNDILKEFM |
| | | | VRNTYIYPPQPSMRIISEIFAYTSANMPKWNSISISGYHMQEA |
| | | | GATADIEMAYTLADGVDYIRAGESVGLNVDQFAPRLSFFWGI |
| | | | GMNFFMEVAKLRAARMLWAKLVHQFGPKNPKSMSLRTHSQ |
| | | | TSGWSLTAQDVYNNVVRTCIEAMAATQGHTQSLHTNSLDEA |
| | | | IALPTDFSARIARNTQLFLQQESGTTRVIDPWSGSAYVEELTW |
| | | | DLARKAWGHIQEVEKVGGMAKAIEKGIPKMRIEEAAARTQA |
| | | | RIDSGRQPLIGVNKYRLEHEPPLDVLKVDNSTVLAEQKAKLV |
| | | | KLRAERDPEKVKAALDKITWAAGNPDDKDPDRNLLKLCIDA |
| | | | GRAMATVGEMSDALEKVFGRYTAQIRTISGVYSKEVKNTPE |
| | | | VEEARELVEEFEQAEGRRPRILLAKMGQDGHDRGQKVIATA |
| | | | YADLGFDVDVGPLFQTPEETARQAVEADVHVVGVSSLAGGH |
| | | | LTLVPALRKELDKLGRPDILITVGGVIPEQDFDELRKDGAVEI |
| | | | YTPGTVIPESAISLVKKLRASLDA |
| 112 | mutab | Propionibacterium | GTGAGCACTCTGCCCCGTTTTGATTCAGTTGACCTCGGCAA |
| | | freudenreichii | TGCCCCGGTTCCTGCTGATGCCGCACGACGCTTCGAGGAA |
| | | subsp. | CTGGCCGCCAAGGCCGGCACCGGAGAGGCGTGGGAGACG |
| | | shermanii | GCCGAGCAGATTCCGGTTGGCACCCTGTTCAACGAAGACG |
| | | | TCTACAAGGACATGGACTGGCTGGACACCTACGCAGGTAT |
| | | | CCCGCCGTTCGTCCACGGCCCGTATGCAACCATGTACGCG |
| | | | TTCCGTCCCTGGACGATTCGCCAGTACGCCGGTTTCTCCAC |
| | | | GGCCAAGGAGTCGAACGCCTTCTACCGCCGCAACCTTGCG |
| | | | GCCGGCCAGAAGGGCCTGTCGGTTGCCTTCGACCTGCCCA |
| | | | CCCACCGTGGCTACGACTCGGACAATCCCCGCGTCGCCGG |
| | | | TGACGTCGGCATGGCCGGTGTGGCCATCGACTCCATCTAT |
| | | | GACATGCGCGAGCTGTTCGCCGGCATTCCGCTGGACCAGA |
| | | | TGAGCGTGTCCATGACCATGAACGGCGCCGTGCTGCCGAT |
| | | | CCTGGCCCTCTATGTGGTGACCGCCGAGGAGCAGGGCGTC |
| | | | AAGCCCGAGCAGCTCGCCGGGACGATCCAGAACGACATC |
| | | | CTCAAGGAGTTCATGGTTCGTAACACCTACATCTACCCGC |
| | | | CGCAGCCGAGTATGCGAATCATCTCTGAGATCTTCGCCTA |
| | | | CACGAGTGCCAATATGCCGAAGTGGAATTCGATTTCCATT |
| | | | TCCGGCTACCACATGCAGGAAGCCGGCGCCACGGCCGACA |
| | | | TCGAGATGGCCTATACCCTGGCCGACGGTGTTGACTACAT |
| | | | CCGCGCCGGCGAGTCGGTGGGCCTCAATGTCGACCAGTTC |
| | | | GCGCCGCGTCTGTCCTTCTTCTGGGGCATCGGCATGAACTT |
| | | | CTTCATGGAGGTTGCCAAGCTGCGTGCCGCGCGCATGTTG |
| | | | TGGGCCAAGCTGGTGCATCAGTTCGGGCCGAAGAACCCGA |
| | | | AGTCGATGAGCCTGCGCACCCACTCGCAGACCTCCGGTTG |
| | | | GTCGCTGACCGCCCAGGACGTCTACAACAACGTCGTGCGT |
| | | | ACCTGCATCGAGGCCATGGCCGCCACCCAGGGCCATACCC |
| | | | AGTCGCTGCACACGAACTCGCTCGACGAGGCCATCGCCCT |
| | | | GCCGACCGATTTCAGCGCCCGCATCGCCCGTAACACCCAG |
| | | | CTGTTCCTGCAGCAGGAATCGGGCACGACGCGCGTGATCG |
| | | | ACCCGTGGAGCGGCTCGGCATACGTCGAGGAGCTCACCTG |
| | | | GGACCTGGCCCGCAAGGCATGGGGTCACATCCAGGAGGTC |
| | | | GAGAAGGTCGGCGGCATGGCCAAGGCCATCGAAAAGGGC |
| | | | ATCCCCAAGATGCGCATCGAGGAAGCCGCCGCCCGCACCC |
| | | | AGGCACGCATCGACTCCGGCCGCCAGCCGCTGATCGGCGT |
| | | | GAACAAGTACCGCCTGGAGCACGAGCCGCCGCTCGATGTG |
| | | | CTCAAGGTGGACAACTCCACGGTGCTCGCCGAGCAGAAGG |
| | | | CCAAGCTGGTCAAGCTGCGCGCCGAGCGCGATCCCGAGAA |
| | | | GGTCAAGGCCGCCCTCGACAAGATCACCTGGGCCGCCGGC |
| | | | AACCCCGACGACAAGGATCCGGATCGCAACCTGCTGAAGC |
| | | | TGTGCATCGACGCTGGCCGCGCCATGGCGACGGTCGGCGA |
| | | | GATGAGCGACGCGCTCGAGAAGGTCTTCGGACGCTACACC |
| | | | GCCCAGATTCGCACCATCTCCGGTGTGTACTCGAAGGAAG |
| | | | TGAAGAACACGCCTGAGGTTGAGGAAGCACGCGAGCTCG |
| | | | TTGAGGAATTCGAGCAGGCCGAGGGCCGTCGTCCTCGCAT |
| | | | CCTGCTGGCCAAGATGGGCCAGGACGGTCACGACCGTGGC |
| | | | CAGAAGGTCATCGCCACCGCCTATGCCGACCTCGGTTTCG |
| | | | ACGTCGACGTGGGCCCGCTGTTCCAGACCCCGGAGGAGAC |
| | | | CGCACGTCAGGCCGTCGAGGCCGATGTGCACGTGGTGGGC |
| | | | GTTTCGTCGCTCGCCGGCGGGCATCTGACGCTGGTTCCGG |
| | | | CCCTGCGCAAGGAGCTGGACAAGCTCGGACGTCCCGACAT |
| | | | CCTCATCACCGTGGGCGGCGTGATCCCTGAGCAGGACTTC |
| | | | GACGAGCTGCGTAAGGACGGCGCCGTGGAGATCTACACCC |
| | | | CCGGCACCGTCATTCCGGAGTCGGCGATCTCGCTGGTCAA |
| | | | GAAACTGCGGGCTTCGCTCGATGCCTAG |
| 113 | CobB | E. coli | MEKPRVLVLTGAGISAESGIRTFRAADGLWEEHRVEDVATPE |
| | | | GFDRDPELVQAFYNARRRQLQQPEIQPNAAHLALAKLQDAL |
| | | | GDRFLLVTQNIDNLHERAGNTNVIHMHGELLKVRCSQSGQV |
| | | | LDWTGDVTPEDKCHCCQFPAPLRPHVVWFGEMPLGMDEIY |
| | | | MALSMADIFIAIGTSGHVYPAAGFVHEAKLHGAHTVELNLEP |
| | | | SQVGNEFAEKYYGPASQVVPEFVEKLLKGLKAGSIA |
| 114 | cobB | E. coli | ATGGAAAAACCAAGAGTACTCGTACTGACAGGGGCAGGA |
| | | | ATTTCTGCGGAATCAGGTATTCGTACCTTTCGCGCCGCAGA |
| | | | TGGCCTGTGGGAAGAACATCGGGTTGAAGATGTGGCAACG |
| | | | CCGGAAGGTTTCGATCGCGATCCTGAACTGGTGCAAGCGT |
| | | | TTTATAACGCCCGTCGTCGACAGCTGCAGCAGCCAGAAAT |
| | | | TCAGCCTAACGCCGCGCATCTTGCGCTGGCTAAACTGCAA |
| | | | GACGCCCTCGGCGATCGCTTTTTGCTGGTGACGCAGAATA |
| | | | TCGACAACCTGCATGAACGCGCAGGTAATACCAATGTGAT |
| | | | TCATATGCATGGGGAACTGCTGAAAGTGCGTTGTTCACAA |
| | | | AGTGGTCAGGTTCTCGACTGGACAGGAGACGTTACCCCAG |
| | | | AAGATAAATGCCATTGTTGCCAGTTTCCGGCACCCTTGCG |
| | | | CCCGCACGTAGTGTGGTTTGGCGAAATGCCACTCGGCATG |
| | | | GATGAAATTTATATGGCGTTGTCGATGGCCGATATTTTCAT |
| | | | TGCCATTGGCACATCCGGGCATGTTTATCCGGCGGCTGGG |
| | | | TTTGTTCACGAAGCGAAACTGCATGGCGCGCACACCGTGG |
| | | | AACTGAATCTTGAACCGAGTCAGGTTGGTAATGAATTTGC |
| | | | CGAGAAATATTACGGCCCGGCAAGCCAGGTGGTGCCTGAG |
| | | | TTTGTTGAAAAGTTGCTGAAGGGATTAAAAGCGGGAAGCA |
| | | | TTGCCTGA |
| 115 | Pka | E. coli | MSQRGLEALLRPKSIAVIGASMKPNRAGYLMMRNLLAGGFN |
| | | | GPVLPVTPAWKAVLGVLAWPDIASLPFTPDLAVLCTNASRNL |
| | | | ALLEELGEKGCKTCIILSAPASQHEDLRACALRHNMRLLGPN |
| | | | SLGLLAPWQGLNASFSPVPIKRGKLAFISQSAAVSNTILDWAQ |
| | | | QRKMGFSYFIALGDSLDIDVDELLDYLARDSKTSAILLYLEQL |
| | | | SDARRFVSAARSASRNKPILVIKSGRSPAAQRLLNTTAGMDP |
| | | | AWDAAIQRAGLLRVQDTHELFSAVETLSHMRPLRGDRLMIIS |
| | | | NGAAPAALALDALWSRNGKLATLSEETCQKLRDALPEHVAI |
| | | | SNPLDLRDDASSEHYIKTLDILLHSQDFDALMVIHSPSAAAPA |
| | | | TESAQVLIEAVKHHPRSKYVSLLTNWCGEHSSQEARRLFSEA |
| | | | GLPTYRTPEGTITAFMHMVEYRRNQKQLRETPALPSNLTSNT |
| | | | AEAHLLLQQAIAEGATSLDTHEVQPILQAYGMNTLPTWIASD |
| | | | STEAVHIAEQIGYPVALKLRSPDIPHKSEVQGVMLYLRTANE |
| | | | VQQAANAIFDRVKMAWPQARVHGLLVQSMANRAGAQELR |
| | | | VVVEHDPVFGPLIMLGEGGVEWRPEDQAVVALPPLNMNLAR |
| | | | YLVIQGIKSKKIRARSALRPLDVAGLSQLLVQVSNLIVDCPEI |
| | | | QRLDIHPLLASGSEFTALDVTLDISPFEGDNESRLAVRPYPHQ |
| | | | LEEWVELKNGERCLFRPILPEDEPQLQQFISRVTKEDLYYRYF |
| | | | SEINEFTHEDLANMTQIDYDREMAFVAVRRIDQTEEILGVTR |
| | | | AISDPDNIDAEFAVLVRSDLKGLGLGRRLMEKLITYTRDHGL |
| | | | QRLNGITMPNNRGMVALARKLGFNVDIQLEEGIVGLTLNLA |
| | | | QREES |
| 116 | pka | E. coli | ATGAGTCAGCGAGGACTGGAAGCACTACTGCGACCAAAA |
| | | | TCGATAGCGGTAATTGGCGCGTCGATGAAACCCAATCGCG |
| | | | CAGGTTACCTGATGATGCGTAACCTGCTGGCGGGAGGCTT |
| | | | TAACGGACCGGTACTCCCGGTGACGCCAGCCTGGAAAGCG |
| | | | GTGTTGGGTGTGTTGGCCTGGCCGGATATTGCCAGCTTGCC |
| | | | CTTTACACCCGACCTTGCGGTTTTATGTACCAATGCCAGCC |
| | | | GTAATCTTGCTCTTCTGGAAGAGCTCGGCGAGAAAGGCTG |
| | | | TAAAACCTGCATTATTCTTTCCGCCCCGGCATCGCAACACG |
| | | | AAGATCTCCGCGCCTGCGCCCTGCGCCATAACATGCGCCT |
| | | | GCTTGGACCAAACAGTCTGGGTTTACTGGCTCCCTGGCAA |
| | | | GGTCTGAATGCCAGCTTTTCGCCTGTGCCGATTAAACGCG |
| | | | GCAAGCTGGCGTTTATTTCGCAATCGGCTGCCGTCTCCAAC |
| | | | ACCATCCTCGACTGGGCGCAACAGCGTAAGATGGGCTTTT |
| | | | CCTACTTTATTGCGCTCGGCGACAGCCTGGATATCGACGTT |
| | | | GATGAATTGCTTGACTATCTGGCACGCGACAGTAAAACCA |
| | | | GCGCCATCCTGCTCTATCTCGAACAGTTAAGCGACGCGCG |
| | | | ACGCTTTGTTTCGGCGGCCCGTAGTGCCTCGCGTAATAAA |
| | | | CCGATTCTGGTGATTAAAAGCGGACGTAGCCCGGCGGCAC |
| | | | AGCGACTGCTCAACACGACGGCAGGAATGGACCCGGCAT |
| | | | GGGATGCGGCTATTCAGCGTGCCGGTTTGTTGCGGGTACA |
| | | | GGACACCCACGAGCTGTTTTCGGCGGTGGAAACCCTTAGC |
| | | | CATATGCGCCCGCTACGTGGCGACCGGCTGATGATTATCA |
| | | | GCAACGGTGCTGCGCCTGCCGCGCTGGCGCTGGATGCCTT |
| | | | ATGGTCACGCAATGGCAAGCTGGCAACGCTAAGCGAAGA |
| | | | AACCTGCCAGAAACTGCGCGATGCACTGCCAGAACATGTG |
| | | | GCAATATCTAACCCGCTCGATCTACGCGATGACGCCAGCA |
| | | | GTGAGCACTATATTAAAACGCTGGATATTCTGCTCCACAG |
| | | | CCAGGATTTTGACGCGCTGATGGTTATTCATTCGCCCAGCG |
| | | | CCGCTGCTCCCGCAACAGAAAGCGCGCAAGTATTAATTGA |
| | | | AGCGGTAAAGCATCATCCCCGCAGCAAATATGTCTCTTTG |
| | | | CTGACGAACTGGTGCGGCGAGCACTCCTCGCAAGAGGCAC |
| | | | GACGTTTATTCAGCGAAGCCGGGCTGCCGACCTACCGTAC |
| | | | CCCGGAAGGAACCATCACTGCTTTTATGCATATGGTGGAG |
| | | | TACCGGCGTAATCAGAAGCAACTACGCGAAACGCCGGCGT |
| | | | TGCCCAGCAATCTGACTTCCAATACCGCAGAAGCGCATCT |
| | | | TCTGTTGCAACAGGCGATTGCCGAAGGGGCTACGTCGCTC |
| | | | GATACCCATGAAGTTCAGCCCATCCTGCAAGCGTATGGCA |
| | | | TGAACACGCTCCCTACCTGGATTGCCAGCGATAGCACCGA |
| | | | AGCGGTGCATATTGCCGAACAGATTGGTTATCCGGTGGCG |
| | | | CTGAAATTGCGTTCGCCGGATATTCCACATAAATCGGAAG |
| | | | TTCAGGGCGTCATGCTTTACCTGCGTACAGCCAATGAAGT |
| | | | CCAGCAAGCGGCGAACGCTATTTTCGATCGCGTAAAAATG |
| | | | GCCTGGCCACAGGCGCGGGTCCACGGCCTGTTGGTGCAAA |
| | | | GTATGGCTAACCGTGCTGGCGCTCAGGAGTTGCGGGTTGT |
| | | | GGTTGAGCACGATCCGGTTTTCGGGCCGTTGATCATGCTG |
| | | | GGTGAAGGCGGTGTGGAGTGGCGTCCTGAAGATCAAGCC |
| | | | GTCGTCGCACTGCCGCCGCTGAACATGAACCTGGCCCGCT |
| | | | ATCTGGTTATTCAGGGGATCAAAAGTAAAAAGATTCGTGC |
| | | | GCGCAGTGCGCTACGCCCATTGGATGTTGCAGGCTTGAGC |
| | | | CAGCTTCTGGTGCAGGTTTCCAACTTGATTGTCGATTGCCC |
| | | | GGAAATTCAGCGTCTGGATATTCATCCTTTGCTGGCTTCTG |
| | | | GCAGTGAATTTACCGCGCTGGATGTCACGCTGGATATCTC |
| | | | GCCGTTTGAAGGCGATAACGAGAGTCGGCTGGCAGTGCGC |
| | | | CCTTATCCGCATCAGCTGGAAGAATGGGTAGAATTGAAAA |
| | | | ACGGTGAACGCTGCTTGTTCCGCCCGATTTTGCCAGAAGA |
| | | | TGAGCCACAACTTCAGCAATTCATTTCGCGAGTCACCAAA |
| | | | GAAGATCTTTATTACCGCTACTTTAGCGAGATCAACGAAT |
| | | | TTACCCATGAAGATTTAGCCAACATGACACAGATCGACTA |
| | | | CGATCGGGAAATGGCGTTTGTAGCGGTACGACGTATTGAT |
| | | | CAAACGGAAGAGATCCTCGGCGTCACGCGTGCGATTTCCG |
| | | | ATCCTGATAACATCGATGCCGAATTTGCTGTACTGGTTCGC |
| | | | TCGGATCTCAAAGGGTTAGGCTTAGGTCGACGCTTAATGG |
| | | | AAAAGTTGATTACCTATACGCGAGATCACGGACTACAACG |
| | | | TCTGAATGGTATTACGATGCCAAACAATCGTGGCATGGTG |
| | | | GCGCTAGCCCGCAAGCTCGGGTTTAACGTTGATATCCAGC |
| | | | TCGAAGAGGGGATCGTTGGGCTTACGCTAAATCTTGCCCA |
| | | | GCGCGAGGAATCATGA |
| 117 | DcuC | E. coli | MLTFIELLIGVVVIVGVARYIIKGYSATGVLFVGGLLLLIISAI |
| | | | MGHKVLPSSQASTGYSATDIVEYVKILLMSRGGDLGMMIMM |
| | | | LCGFAAYMTHIGANDMVVKLASKPLQYINSPYLLMIAAYFV |
| | | | ACLMSLAVSSATGLGVLLMATLFPVMVNVGISRGAAAAICA |
| | | | SPAAIILAPTSGDVVLAAQASEMSLIDFAFKTTLPISIAAIIGMA |
| | | | IAHFFWQRYLDKKEHISHEMLDVSEITTTAPAFYAILPFTPIIG |
| | | | VLIFDGKWGPQLHIITILVICMLIASILEFLRSFNTQKVFSGLEV |
| | | | AYRGMADAFANVVMLLVAAGVFAQGLSTIGFIQSLISIATSF |
| | | | GSASIILMLVLVILTMLAAVTTGSGNAPFYAFVEMIPKLAHSS |
| | | | GINPAYLTIPMLQASNLGRTLSPVSGVVVAVAGMAKISPFEV |
| | | | VKRTSVPVLVGLVIVIVATELMVPGTAAAVTGK |
| 118 | dcuc | E. coli | ATGCTGACATTCATTGAGCTCCTTATTGGGGTTGTGGTTAT |
| | | | TGTGGGTGTAGCTCGCTACATCATTAAAGGGTATTCCGCC |
| | | | ACTGGTGTGTTATTTGTCGGTGGCCTGTTATTGCTGATTAT |
| | | | CAGTGCCATTATGGGGCACAAAGTGTTACCGTCCAGCCAG |
| | | | GCTTCAACAGGCTACAGCGCCACGGATATCGTTGAATACG |
| | | | TTAAAATATTACTAATGAGCCGCGGCGGCGACCTCGGCAT |
| | | | GATGATTATGATGCTGTGTGGATTTGCCGCTTACATGACCC |
| | | | ATATCGGCGCGAATGATATGGTGGTCAAGCTGGCGTCAAA |
| | | | ACCATTGCAGTATATTAACTCCCCTTACCTGCTGATGATTG |
| | | | CCGCCTATTTTGTCGCCTGTCTGATGTCTCTGGCCGTCTCTT |
| | | | CCGCAACCGGTCTGGGTGTTTTGCTGATGGCAACCCTATTT |
| | | | CCGGTGATGGTAAACGTTGGTATCAGTCGTGGCGCAGCTG |
| | | | CTGCCATTTGTGCCTCCCCGGCGGCGATTATTCTCGCACCG |
| | | | ACTTCAGGGGATGTGGTGCTGGCGGCGCAAGCTTCCGAAA |
| | | | TGTCGCTGATTGACTTCGCCTTCAAAACGACGCTGCCTATC |
| | | | TCAATTGCTGCAATTATCGGCATGGCGATCGCCCACTTCTT |
| | | | CTGGCAACGTTATCTGGATAAAAAAGAGCACATCTCTCAT |
| | | | GAAATGTTAGATGTCAGTGAAATCACCACCACTGCTCCTG |
| | | | CGTTTTATGCCATTTTGCCGTTCACGCCGATCATCGGTGTA |
| | | | CTGATTTTTGACGGTAAATGGGGTCCGCAATTACACATCA |
| | | | TCACTATTCTGGTGATTTGTATGCTGATTGCCTCCATTCTG |
| | | | GAGTTCCTCCGCAGCTTTAATACCCAGAAAGTTTTCTCTGG |
| | | | TCTGGAAGTGGCTTATCGCGGGATGGCAGATGCGTTTGCT |
| | | | AACGTGGTGATGCTGCTGGTTGCCGCTGGGGTATTCGCTC |
| | | | AGGGGCTTAGCACCATCGGCTTTATTCAAAGTCTGATTTCT |
| | | | ATCGCTACCTCGTTTGGTTCGGCGAGTATCATCCTGATGCT |
| | | | GGTATTGGTGATTCTGACAATGCTGGCGGCAGTCACGACC |
| | | | GGTTCAGGCAATGCGCCGTTTTATGCGTTTGTTGAGATGAT |
| | | | CCCGAAACTGGCGCACTCTTCCGGCATTAACCCGGCGTAT |
| | | | TTGACTATCCCGATGCTGCAGGCGTCAAACCTTGGCCGTA |
| | | | CCCTTTCGCCCGTTTCTGGCGTAGTCGTTGCGGTTGCCGGG |
| | | | ATGGCGAAGATCTCGCCGTTTGAAGTCGTAAAACGCACCT |
| | | | CGGTACCGGTGCTTGTTGGTTTGGTGATTGTTATCGTTGCT |
| | | | ACAGAGCTGATGGTGCCAGGAACGGCAGCAGCGGTCACA |
| | | | GGCAAGTAA |
| 119 | SucE1 | Corynebactrium | MTVGLLLGRIKIFGFRLGVAAVLFVGLALSTIEPDISVPSLIYV |
| | | glutamicum | VGLSLFVYTIGLEAGPGFFTSMKTTGLRNNALTLGAIIATTAL |
| | | | AWALITVLNIDAASGAGMLTGALTNTPAMAAVVDALPSLID |
| | | | DTGQLHLIAELPVVAYSLAYPLGVLIVILSIAIFSSVFKVDHNK |
| | | | EAEEAGVAVQELKGRRIRVTVADLPALENIPELLNLHVIVSRV |
| | | | ERDGEQFIPLYGEHARIGDVLTVVGADEELNRAEKAIGELIDG |
| | | | DPYSNVELDYRRIFVSNTAVVGTPLSKLQPLFKDMLITRIRRG |
| | | | DTDLVASSDMTLQLGDRVRVVAPAEKLREATQLLGDSYKKL |
| | | | SDFNLLPLAAGLMIGVLVGMVEFPLPGGSSLKLGNAGGPLVV |
| | | | ALLLGMINRTGKFVWQIPYGANLALRQLGITLFLAAIGTSAG |
| | | | AGFRSAISDPQSLTIIGFGALLTLFISITVLFVGHKLMKIPFGET |
| | | | AGILAGTQTHPAVLSYVSDASRNELPAMGYTSVYPLAMIAKI |
| | | | LAAQTLLFLLI |
| 120 | sucE1 | Corynebactrium | GTGAGCTTCCTTGTAGAAAATCAATTACTCGCGTTGGTTGT |
| | | glutamicum | CATCATGACGGTCGGACTATTGCTCGGCCGCATCAAAATT |
| | | | TTCGGGTTCCGTCTCGGCGTCGCCGCTGTACTGTTTGTAGG |
| | | | TCTAGCGCTATCCACCATTGAGCCGGATATTTCCGTCCCAT |
| | | | CCCTCATTTACGTGGTTGGACTGTCGCTTTTTGTCTACACG |
| | | | ATCGGTCTGGAAGCCGGCCCTGGATTCTTCACCTCCATGA |
| | | | AAACCACTGGTCTGCGCAACAACGCACTGACCTTGGGCGC |
| | | | CATCATCGCCACCACGGCACTCGCATGGGCACTCATCACA |
| | | | GTTTTGAACATCGATGCCGCCTCCGGCGCCGGCATGCTCA |
| | | | CCGGCGCGCTCACCAACACCCCAGCCATGGCCGCAGTTGT |
| | | | TGACGCACTTCCTTCGCTTATCGACGACACCGGCCAGCTTC |
| | | | ACCTCATCGCCGAGCTGCCCGTCGTCGCATATTCCTTGGCA |
| | | | TACCCCCTCGGTGTGCTCATCGTTATTCTCTCCATCGCCAT |
| | | | CTTCAGCTCTGTGTTCAAAGTCGACCACAACAAAGAAGCC |
| | | | GAAGAAGCGGGCGTTGCGGTCCAGGAACTCAAAGGCCGT |
| | | | CGCATCCGCGTCACCGTCGCTGATCTTCCAGCCCTGGAGA |
| | | | ACATCCCAGAGCTGCTCAACCTCCACGTCATTGTGTCCCG |
| | | | AGTGGAACGAGACGGTGAGCAATTCATCCCGCTTTATGGC |
| | | | GAACACGCACGCATCGGCGATGTCTTAACAGTGGTGGGTG |
| | | | CCGATGAAGAACTCAACCGCGCGGAAAAAGCCATCGGTG |
| | | | AACTCATTGACGGCGACCCCTACAGCAATGTGGAACTTGA |
| | | | TTACCGACGCATCTTCGTCTCAAACACAGCAGTCGTGGGC |
| | | | ACTCCCCTATCCAAGCTCCAGCCACTGTTTAAAGACATGCT |
| | | | GATCACCCGCATCAGGCGCGGCGACACAGATTTGGTGGCC |
| | | | TCCTCCGACATGACTTTGCAGCTCGGTGACCGTGTCCGCGT |
| | | | TGTCGCACCAGCAGAAAAACTCCGCGAAGCAACCCAATTG |
| | | | CTCGGCGATTCCTACAAGAAACTCTCCGATTTCAACCTGCT |
| | | | CCCACTCGCTGCCGGCCTCATGATCGGTGTGCTTGTCGGCA |
| | | | TGGTGGAGTTCCCACTACCAGGTGGAAGCTCCCTGAAACT |
| | | | GGGTAACGCAGGTGGACCGCTAGTTGTTGCGCTGCTGCTC |
| | | | GGCATGATCAATCGCACAGGCAAGTTCGTCTGGCAAATCC |
| | | | CCTACGGAGCAAACCTTGCCCTTCGCCAACTGGGCATCAC |
| | | | ACTATTTTTGGCTGCCATCGGTACCTCAGCGGGCGCAGGA |
| | | | TTTCGATCAGCGATCAGCGACCCCCAATCACTCACCATCA |
| | | | TCGGCTTCGGTGCGCTGCTCACTTTGTTCATCTCCATCACG |
| | | | GTGCTGTTCGTTGGCCACAAACTGATGAAAATCCCCTTCG |
| | | | GTGAAACCGCTGGCATCCTCGCCGGTACGCAAACCCACCC |
| | | | TGCTGTGCTGAGTTATGTGTCAGATGCCTCCCGCAACGAG |
| | | | CTCCCTGCCATGGGTTATACCTCTGTGTATCCGCTGGCGAT |
| | | | GATCGCAAAGATCCTGGCCGCCCAAACGTTGTTGTTCCTA |
| | | | CTTATCTAG |
| 121 | DuaA | E. coli | MNKIFSSHVMPFRALIDACWKEKYTAARFTRDLIAGITVGIIA |
| | | | IPLAMALAIGSGVAPQYGLYTAAVAGIVIALTGGSRFSVSGPT |
| | | | AAFVVILYPVSQQFGLAGLLVATLLSGIFLILMGLARFGRLIE |
| | | | YIPVSVTLGFTSGIGITIGTMQIKDFLGLQMAHVPEHYLQKVG |
| | | | ALFMALPTINVGDAAIGIVTLGILVFWPRLGIRLPGHLPALLA |
| | | | GCAVMGIVNLLGGHVATIGSQFHYVLADGSQGNGIPQLLPQL |
| | | | VLPWDLPNSEFTLTWDSIRTLLPAAFSMAMLGAIESLLCAVV |
| | | | LDGMTGTKHKANSELVGQGLGNIIAPFFGGITATAAIARSAA |
| | | | NVRAGATSPISAVIHSILVILALLVLAPLLSWLPLSAMAALLL |
| | | | MVAWNMSEAHKVVDLLRHAPKDDIIVMLLCMSLTVLFDMV |
| | | | IAISVGIVLASLLFMRRIARMTRLAPVVVDVPDDVLVLRVIGP |
| | | | LFFAAAEGLFTDLESRLEGKRIVILKWDAVPVLDAGGLDAFQ |
| | | | RFVKRLPEGCELRVCNVEFQPLRTMARAGIQPIPGRLAFFPNR |
| | | | RAAMADL |
| 122 | duaA | E. coli | GTGAACAAAATATTTTCCTCACATGTGATGCCTTTCCGCGC |
| | | | TCTGATCGACGCTTGCTGGAAAGAAAAATATACTGCCGCA |
| | | | CGGTTTACCCGTGACCTGATTGCCGGGATAACCGTCGGGA |
| | | | TTATTGCTATCCCGCTGGCGATGGCGTTGGCTATTGGTAGT |
| | | | GGTGTGGCACCCCAGTACGGTTTATATACCGCAGCTGTTG |
| | | | CGGGGATTGTCATTGCTCTGACGGGTGGGTCACGCTTTAG |
| | | | CGTTTCCGGTCCGACTGCGGCATTTGTGGTAATTCTCTATC |
| | | | CCGTGTCGCAACAGTTTGGACTGGCAGGACTGCTGGTTGC |
| | | | GACCTTGCTGTCGGGGATCTTTTTGATTCTGATGGGTCTGG |
| | | | CACGCTTTGGTCGCCTGATTGAGTATATTCCGGTTTCCGTC |
| | | | ACCTTAGGTTTCACCTCGGGTATCGGGATCACCATCGGTA |
| | | | CCATGCAGATTAAAGATTTTCTCGGTCTGCAAATGGCCCA |
| | | | TGTCCCGGAACATTATCTACAAAAAGTCGGCGCATTATTT |
| | | | ATGGCGCTGCCGACCATTAATGTGGGTGATGCTGCCATTG |
| | | | GCATTGTGACGCTAGGTATTCTTGTTTTTTGGCCGCGTCTG |
| | | | GGCATTCGTTTACCCGGTCACCTTCCGGCCTTGCTGGCTGG |
| | | | TTGCGCGGTGATGGGGATTGTTAACCTGCTCGGCGGACAT |
| | | | GTTGCTACCATCGGTTCGCAATTCCACTACGTCCTGGCCGA |
| | | | TGGTTCTCAGGGTAACGGTATTCCGCAACTGCTGCCGCAA |
| | | | CTGGTGCTGCCGTGGGATCTGCCTAATTCAGAATTCACGCT |
| | | | AACCTGGGATTCTATTCGCACACTGCTGCCTGCGGCATTCT |
| | | | CAATGGCAATGCTCGGCGCAATCGAATCTCTGCTCTGCGC |
| | | | CGTGGTGCTGGATGGTATGACCGGGACGAAACACAAGGC |
| | | | GAACAGCGAACTGGTTGGACAGGGACTGGGGAATATTATC |
| | | | GCTCCGTTCTTTGGTGGTATTACCGCTACAGCTGCCATCGC |
| | | | GCGTTCTGCCGCTAACGTCCGTGCCGGGGCAACGTCCCCT |
| | | | ATCTCGGCGGTGATCCACTCTATTCTGGTTATTCTTGCCCT |
| | | | GCTGGTACTGGCACCGCTGCTCTCCTGGCTGCCGCTTTCCG |
| | | | CCATGGCAGCCCTGCTGTTGATGGTGGCGTGGAACATGAG |
| | | | TGAAGCGCACAAAGTGGTCGACTTGCTGCGTCATGCGCCG |
| | | | AAAGATGACATCATCGTCATGCTGCTGTGCATGTCGCTGA |
| | | | CCGTGTTGTTTGATATGGTTATTGCCATCAGCGTGGGGATC |
| | | | GTGCTGGCATCGCTGCTGTTTATGCGTCGTATCGCACGTAT |
| | | | GACTCGCCTGGCACCGGTAGTCGTAGATGTTCCAGACGAT |
| | | | GTCCTGGTTCTGCGCGTTATTGGCCCGCTGTTTTTTGCTGC |
| | | | TGCTGAAGGCTTATTCACGGACCTGGAGTCACGTCTTGAA |
| | | | GGCAAACGGATTGTGATTCTGAAGTGGGATGCCGTTCCGG |
| | | | TACTTGATGCTGGTGGTCTTGATGCGTTCCAGCGTTTTGTG |
| | | | AAGCGTCTGCCCGAGGGATGTGAACTGCGCGTGTGCAACG |
| | | | TGGAATTCCAGCCACTGCGCACTATGGCTCGCGCTGGCAT |
| | | | TCAACCGATCCCGGGACGCCTGGCGTTCTTCCCGAATCGT |
| | | | CGCGCGGCGATGGCGGATTTATAA |
| 123 | DctA | E. coli | MKTSLFKSLYFQVLTAIAIGILLGHFYPEIGEQMKPLGDGFVK |
| | | | LIKMIIAPVIFCTVVTGIAGMESMKAVGRTGAVALLYFEIVSTI |
| | | | ALIIGLIIVNVVQPGAGMNVDPATLDAKAVAVYADQAKDQG |
| | | | IVAFIMDVIPASVIGAFASGNILQVLLFAVLFGFALHRLGSKG |
| | | | QLIFNVIESFSQVIFGIINMIMRLAPIGAFGAMAFTIGKYGVGT |
| | | | LVQLGQLIICFYITCILFVVLVLGSIAKATGFSIFKFIRYIREELL |
| | | | IVLGTSSSESALPRMLDKMEKLGCRKSVVGLVIPTGYSFNLD |
| | | | GTSIYLTMAAVFIAQATNSQMDIVHQITLLIVLLLSSKGAAGV |
| | | | TGSGFIVLAATLSAVGHLPVAGLALILGIDRFMSEARALTNLV |
| | | | GNGVATIVVAKWVKELDHKKLDDVLNNRAPDGKTHELSS |
| 124 | dctA | E. coli | ATGAAAACCTCTCTGTTTAAAAGCCTTTACTTTCAGGTCCT |
| | | | GACAGCGATAGCCATTGGTATTCTCCTTGGCCATTTCTATC |
| | | | CTGAAATAGGCGAGCAAATGAAACCGCTTGGCGACGGCTT |
| | | | CGTTAAGCTCATTAAGATGATCATCGCTCCTGTCATCTTTT |
| | | | GTACCGTCGTAACGGGCATTGCGGGCATGGAAAGCATGAA |
| | | | GGCGGTCGGTCGTACCGGCGCAGTCGCACTGCTTTACTTT |
| | | | GAAATTGTCAGTACCATCGCGCTGATTATTGGTCTTATCAT |
| | | | CGTTAACGTCGTGCAGCCTGGTGCCGGAATGAACGTCGAT |
| | | | CCGGCAACGCTTGATGCGAAAGCGGTAGCGGTTTACGCCG |
| | | | ATCAGGCGAAAGACCAGGGCATTGTCGCCTTCATTATGGA |
| | | | TGTCATCCCGGCGAGCGTCATTGGCGCATTTGCCAGCGGT |
| | | | AACATTCTGCAGGTGCTGCTGTTTGCCGTACTGTTTGGTTT |
| | | | TGCGCTCCACCGTCTGGGCAGCAAAGGCCAACTGATTTTT |
| | | | AACGTCATCGAAAGTTTCTCGCAGGTCATCTTCGGCATCAT |
| | | | CAATATGATCATGCGTCTGGCACCTATTGGTGCGTTCGGG |
| | | | GCAATGGCGTTTACCATCGGTAAATACGGCGTCGGCACAC |
| | | | TGGTGCAACTGGGGCAGCTGATTATCTGTTTCTACATTACC |
| | | | TGTATCCTGTTTGTGGTGCTGGTATTGGGTTCAATCGCTAA |
| | | | AGCGACTGGTTTCAGTATCTTCAAATTTATCCGCTACATCC |
| | | | GTGAAGAACTGCTGATTGTACTGGGGACTTCATCTTCCGA |
| | | | GTCGGCGCTGCCGCGTATGCTCGACAAGATGGAGAAACTC |
| | | | GGCTGCCGTAAATCGGTGGTGGGGCTGGTCATCCCGACAG |
| | | | GCTACTCGTTTAACCTTGATGGCACATCGATATACCTGACA |
| | | | ATGGCGGCGGTGTTTATCGCCCAGGCCACTAACAGTCAGA |
| | | | TGGATATCGTCCACCAAATCACGCTGTTAATCGTGTTGCTG |
| | | | CTTTCTTCTAAAGGGGCGGCAGGGGTAACGGGTAGTGGCT |
| | | | TTATCGTGCTGGCGGCGACGCTCTCTGCGGTGGGCCATTTG |
| | | | CCGGTAGCGGGTCTGGCGCTGATCCTCGGTATCGACCGCT |
| | | | TTATGTCAGAAGCTCGTGCGCTGACTAACCTGGTCGGTAA |
| | | | CGGCGTAGCGACCATTGTCGTTGCTAAGTGGGTGAAAGAA |
| | | | CTGGACCACAAAAAACTGGACGATGTGCTGAATAATCGTG |
| | | | CGCCGGATGGCAAAACGCACGAATTATCCTCTTAA |
| 125 | ClbA | | MRIDILIGHTSFFHQTSRDNFLHYLNEEEIKRYDQFHFVSDKE |
| | | | LYILSRILLKTALKRYQPDVSLQSWQFSTCKYGKPFIVFPQLA |
| | | | KKIFFNLSHTIDTVAVAISSHCELGVDIEQIRDLDNSYLNISQH |
| | | | FFTPQEATNIVSLPRYEGQLLFWKMWTLKEAYIKYRGKGLSL |
| | | | GLDCIEFHLTNKKLTSKYRGSPVYFSQWKICNSFLALASPLIT |
| | | | PKITIELFPMQSQLYHHDYQLIHSSNGQN |
| 126 | clbA | | caaatatcacataatcttaacatatcaataaacacagtaaagtttcatgtgaaaaacatcaaacataa |
| | | | aatacaagctcggaatacgaatcacgctatacacattgctaacaggaatgagattatctaaatgagga |
| | | | ttgatatattaattggacatactagtttttttcatcaaaccagtagagataacttccttcactatctc |
| | | | aatgaggaagaaataaaacgctatgatcagtttcattttgtgagtgataaagaactctatatttta |
| | | | agccgtatcctgctcaaaacagcactaaaaagatatcaacctgatgtctcattacaatcatggcaat |
| | | | ttagtacgtgcaaatatggcaaaccatttatagtttttcctcagttggcaaaaaagattttttttaac |
| | | | ctttcccatactatagatacagtagccgttgctattagttctcactgcgagcttggtgtcgatattga |
| | | | acaaataagagatttagacaactcttatctgaatatcagtcagcatttttttactccacaggaa |
| | | | gctactaacatagtttcacttcctcgttatgaaggtcaattacttttttggaaaatgtggacgct |
| | | | caaagaagcttacatcaaatatcgaggtaaaggcctatctttaggactggattgtattgaa |
| | | | tttcatttaacaaataaaaaactaacttcaaaatatagaggttcacctgtttatttctctcaat |
| | | | ggaaaatatgtaactcatttctcgcattagcctctccactcatcacccctaaaataactat |
| | | | tgagctatttcctatgcagtcccaactttatcaccacgactatcagctaattcattcgtcaa |
| | | | atgggcagaattgaatcgccacggataatctagacacttctgagccgtcgataatat |
| | | | tgattttcatattccgtcggtggtgtaagtatcccgcataatcgtgccattcacatttag |
| 127 | clbA | | ggatggggggaaacatggataagttcaaagaaaaaaacccgttatctctgcgtgaaagacaagt |
| | knockout | | attgcgcatgctggcacaaggtgatgagtactctcaaatatcacataatcttaacatatcaataaac |
| | | | acagtaaagtttcatgtgaaaaacatcaaacataaaatacaagctcggaatacgaatcacgctata |
| | | | cacattgctaacaggaatgagattatctaaatgaggattgaTGTGTAGGCTGGAGC |
| | | | TGCTTCGAAGTTCCTATACTTTCTAGAGAATAGGAACTTCG |
| | | | GAATAGGAACTTCGGAATAGGAACTAAGGAGGATATTCAT |
| | | | ATGtcgtcaaatgggcagaattgaatcgccacggataatctagacacttctgagccgtcgataa |
| | | | tattgattttcatattccgtcggtgg |
| 128 | SucE1 | E. coli | MSFLVENQLLALVVIMTVGLLLGRIKIFGFRLGVA |
| | | | AVLFVGLALSTIEPDISVPSLIYVVGLSLFVYTIGLE |
| | | | AGPGFFTSMKTTGLRNNALTLGAIIATTALAWALI |
| | | | TVLNIDAASGAGMLTGALTNTPAMAAVVDALPSL |
| | | | IDDTGQLHLIAELPVVAYSLAYPLGVLIVILSIAIFSS |
| | | | VFKVDHNKEAEEAGVAVQELKGRRIRVTVADLPA |
| | | | LENIPELLNLHVIVSRVERDGEQFIPLYGEHARIGD |
| | | | VLTVVGADEELNRAEKAIGELIDGDPYSNVELDYR |
| | | | RIFVSNTAVVGTPLSKLQPLFKDMLITRIRRGDTDL |
| | | | VASSDMTLQLGDRVRVVAPAEKLREATQLLGDSY |
| | | | KKLSDFNLLPLAAGLMIGVLVGMVEFPLPGGSSLK |
| | | | LGNAGGPLVVALLLGMINRTGKFVWQIPYGANLA |
| | | | LRQLGITLFLAAIGTSAGAGFRSAISDPQSLTIIGFG |
| | | | ALLTLFISITVLFVGHKLMKIPFGETAGILAGTQTHP |
| | | | AVLSYVSDASRNELPAMGYTSVYPLAMIAKILAA |
| | | | QTLLFLLI |
| 129 | DcuC | E. coli | MLTFIELLIGVVVIVGVARYIIKGYSATGVLFVGGL |
| | | | LLLIISAIMGHKVLPSSQASTGYSATDIVEYVKILL |
| | | | MSRGGDLGMMIMMLCGFAAYMTHIGANDMVVK |
| | | | LASKPLQYINSPYLLMIAAYFVACLMSLAVSSATG |
| | | | LGVLLMATLFPVMVNVGISRGAAAAICASPAAIIL |
| | | | APTSGDVVLAAQASEMSLIDFAFKTTLPISIAAIIGM |
| | | | AIAHFFWQRYLDKKEHISHEMLDVSEITTTAPAFY |
| | | | AILPFTPIIGVLIFDGKWGPQLHIITILVICMLIASILE |
| | | | FIRSFNTQKVFSGLEVAYRGMADAFANVVMLLVA |
| | | | AGVFAQGLSTIGFIQSLISIATSFGSASIILMLVLVIL |
| | | | TMLAAVTTGSGNAPFYAFVEMIPKLAHSSGINPAY |
| | | | LTIPMLQASNLGRTLSPVSGVVVAVAGMAKISPFE |
| | | | VVKRTSVPVLVGLVIVIVATELMVPGTAAAVTGK |
| 130 | accA1 | Streptopmyces | MRKVLIANRGEIAVRVARACRDAGIASVAVYADPDRDALHV |
| | | coelicolor | RAADEAFALGGDTPATSYLDIAKVLKAARESGADAIHPGYGF |
| | | | LSENAEFAQAVLDAGLIWIGPPPHAIRDRGEKVAARHIAQRA |
| | | | GAPLVAGTPDPVSGADEVVAFAKEHGLPIAIKAAFGGGGRGL |
| | | | KVARTLEEVPELYDSAVREAVAAFGRGECFVERYLDKPRHV |
| | | | ETQCLADTHGNVVVVSTRDCSLQRRHQKLVEEAPAPFLSEA |
| | | | QTEQLYSSSKAILKEAGYGGAGTVEFLVGMDGTIFFLEVNTR |
| | | | LQVEHPVTEEVAGIDLVREMFRIADGEELGYDDPALRGHSFE |
| | | | FRINGEDPGRGFLPAPGTVTLFDAPTGPGVRLDAGVESGSVIG |
| | | | PAWDSLLAKLIVTGRTRAEALQRAARALDEFTVEGMATAIPF |
| | | | HRTVVRDPAFAPELTGSTDPFTVHTRWIETEFVNEIKPFTTPA |
| | | | DTETDEESGRETVVVEVGGKRLEVSLPSSLGMSLARTGLAAG |
| | | | ARPKRRAAKKSGPAASGDTLASPMQGTIVKIAVEEGQEVQE |
| | | | GDLIVVLEAMKMEQPLNAHRSGTIKGLTAEVGASLTSGAAIC |
| | | | EIKD |
| 131 | pccB | E. coli | MSEPEEQQPDIHTTAGKLADLRRRIEEATHAGSARAVEKQHA |
| | | | KGKLTARERIDLLLDEGSFVELDEFARHRSTNFGLDANRPYG |
| | | | DGVVTGYGTVDGRPVAVFSQDFTVFGGALGEVYGQKIVKV |
| | | | MDFALKTGCPVVGINDSGGARIQEGVASLGAYGEIFRRNTHA |
| | | | SGVIPQISLVVGPCAGGAVYSPAITDFTVMVDQTSHMFITGPD |
| | | | VIKTVTGEDVGFEELGGARTHNSTSGVAHHMAGDEKDAVE |
| | | | YVKQLLSYLPSNNLSEPPAFPEEADLAVTDEDAELDTIVPDSA |
| | | | NQPYDMHSVIEHVLDDAEFFETQPLFAPNILTGFGRVEGRPV |
| | | | GIVANQPMQFAGCLDITASEKAARFVRTCDAFNVPVLTFVDV |
| | | | PGFLPGVDQEHDGIIRRGAKLIFAYAEATVPLITVITRKAFGG |
| | | | AYDVMGSKHLGADLNLAWPTAQIAVMGAQGAVNILHRRTI |
| | | | ADAGDDAEATRARLIQEYEDALLNPYTAAERGYVDAVIMPS |
| | | | DTRRHIVRGLRQLRTKRESLPPKKHGNIPL |
| 132 | mmcE | Propionibcterium | MSNEDLFICIDHVAYACPDADEASKYYQETFGWHELHREEN |
| | | freudenreichii | PEQGVVEMMAPAAKLTEHMTQVQVMAPLNDESTVAKWLA |
| | | | KHNGRAGLHHMAWRVDDIDAVSATLRERGVQLLYDEPKLG |
| | | | TGGNRINFMHPKSGKGVLIELTQYPKN |
| 133 | mutA | Propionibcterium | MSSTDQGTNPADTDDLTPTTLSLAGDFPKATEEQWEREVEK |
| | | freudenreichii | VLNRGRPPEKQLTFAECLKRLTVHTVDGIDIVPMYRPKDAPK |
| | | | KLGYPGVAPFTRGTTVRNGDMDAWDVRALHEDPDEKFTRK |
| | | | AILEGLERGVTSLLLRVDPDAIAPEHLDEVLSDVLLEMTKVE |
| | | | VFSRYDQGAAAEALVSVYERSDKPAKDLALNLGLDPIAFAA |
| | | | LQGTEPDLTVLGDWVRRLAKFSPDSRAVTIDANIYHNAGAG |
| | | | DVAELAWALATGAEYVRALVEQGFTATEAFDTINFRVTATH |
| | | | DQFLTIARLRALREAWARIGEVFGVDEDKRGARQNAITSWR |
| | | | ELTREDPYVNILRGSIATFSASVGGAESITTLPFTQALGLPEDD |
| | | | FPLRIARNTGIVLAEEVNIGRVNDPAGGSYYVESLTRSLADAA |
| | | | WKEFQEVEKLGGMSKAVMTEHVTKVLDACNAERAKRLAN |
| | | | RKQPITAVSEFPMIGARSIETKPFPAAPARKGLAWHRDSEVFE |
| | | | QLMDRSTSVSERPKVFLACLGTRRDFGGREGFSSPVWHIAGI |
| | | | DTPQVEGGTTAEIVEAFKKSGAQVADLCSSAKVYAQQGLEV |
| | | | AKALKAAGAKALYLSGAFKEFGDDAAEAEKLIDGRLFMGM |
| | | | DVVDTLSSTLDILGVAK |
| 134 | mutB | Propionibcterium | MSTLPRFDSVDLGNAPVPADAARRFEELAAKAGTGEAWETA |
| | | freudenreichii | EQIPVGTLFNEDVYKDMDWLDTYAGIPPFVHGPYATMYAFR |
| | | | PWTIRQYAGFSTAKESNAFYRRNLAAGQKGLSVAFDLPTHR |
| | | | GYDSDNPRVAGDVGMAGVAIDSIYDMRELFAGIPLDQMSVS |
| | | | MTMNGAVLPILALYVVTAEEQGVKPEQLAGTIQNDILKEFM |
| | | | VRNTYIYPPQPSMRIISEIFAYTSANMPKWNSISISGYHMQEA |
| | | | GATADIEMAYTLADGVDYIRAGESVGLNVDQFAPRLSFFWGI |
| | | | GMNFFMEVAKLRAARMLWAKLVHQFGPKNPKSMSLRTHSQ |
| | | | TSGWSLTAQDVYNNVVRTCIEAMAATQGHTQSLHTNSLDEA |
| | | | IALPTDFSARIARNTQLFLQQESGTTRVIDPWSGSAYVEELTW |
| | | | DLARKAWGHIQEVEKVGGMAKAIEKGIPKMRIEEAAARTQA |
| | | | RIDSGRQPLIGVNKYRLEHEPPLDVLKVDNSTVLAEQKAKLV |
| | | | KLRAERDPEKVKAALDKITWAAGNPDDKDPDRNLLKLCIDA |
| | | | GRAMATVGEMSDALEKVFGRYTAQIRTISGVYSKEVKNTPE |
| | | | VEEARELVEEFEQAEGRRPRILLAKMGQDGHDRGQKVIATA |
| | | | YADLGFDVDVGPLFQTPEETARQAVEADVHVVGVSSLAGGH |
| | | | LTLVPALRKELDKLGRPDILITVGGVIPEQDFDELRKDGAVEI |
| | | | YTPGTVIPESAISLVKKLRASLDA |
| 135 | phaB | Acinetobacter | MSEQKVALVTGALGGIGSEICRQLVTAGYKIIATVVPREEDR |
| | | sp | EKQWLQSEGFQDSDVRFVLTDLNNHEAATAAIQEAIAAEGR |
| | | RA3849 | VDVLVNNAGITRDATFKKMSYEQWSQVIDTNLKTLFTVTQP |
| | | | VFNKMLEQKSGRIVNISSVNGLKGQFGQANYSASKAGIIGFT |
| | | | KALAQEGARSNICVNVVAPGYTATPMVTAMREDVIKSIEAQI |
| | | | PLQRLAAPAEIAAAVMYLVSEHGAYVTGETLSINGGLYMH* |
| 136 | phaC | Acinetobacter | MNPNSFQFKENILQFFSVHDDIWKKLQEFYYGQSPINEALAQ |
| | | sp | LNKEDMSLFFEALSKNPARMMEMQWSWWQGQIQIYQNVL |
| | | RA3849 | MRSVAKDVAPFIQPESGDRRFNSPLWQEHPNFDLLSQSYLLF |
| | | | SQLVQNMVDVVEGVPDKVRYRIHFFTRQMINALSPSNFLWT |
| | | | NPEVIQQTVAEQGENLVRGMQVFHDDVMNSGKYLSIRMVN |
| | | | SDSFSLGKDLAYTPGAVVFENDIFQLLQYEATTENVYQTPILV |
| | | | VPPFINKYYVLDLREQNSLVNWLRQQGHTVFLMSWRNPNAE |
| | | | QKELTFADLITQGSVEALRVIEEITGEKEANCIGYCIGGTLLAA |
| | | | TQAYYVAKRLKNHVKSATYMATIIDFENPGSLGVFINEPVVS |
| | | | GLENLNNQLGYFDGRQLAVTFSLLRENTLYWNYYIDNYLKG |
| | | | KEPSDFDILYWNSDGTNIPAKIHNFLLRNLYLNNELISPNAVK |
| | | | VNGVGLNLSRVKTPSFFIATQEDHIALWDTCFRGADYLGGES |
| | | | TLVLGESGHVAGIVNPPSRNKYGCYTNAAKFENTKQWLDGA |
| | | | EYHPESWWLRWQAWVTPYTGEQVPARNLGNAQYPSIEAAP |
| | | | GRYVLVNLF* |
| 137 | phaA | Acinetobacter | MKDVVIVAAKRTAIGSFLGSLASLSAPQLGQTAIRAVLDSAN |
| | | sp | VKPEQVDQVIMGNVLTTGVGQNPARQAAIAAGIPVQVPAST |
| | | RA3849 | LNVVCGSGLRAVHLAAQAIQCDEADIVVAGGQESMSQSAHY |
| | | | MQLRNGQKMGNAQLVDSMVADGLTDAYNQYQMGITAENI |
| | | | VEKLGLNREEQDQLALTSQQRAAAAQAAGKFKDEIAVVSIP |
| | | | QRKGEPVVFAEDEYIKANTSLESLTKLRPAFKKDGSVTAGNA |
| | | | SGINDGAAAVLMMSADKAAELGLKPLARIKGYAMSGIEPEI |
| | | | MGLGPVDAVKKTLNKAGWSLDQVDLIEANEAFAAQALGVA |
| | | | KELGLDLDKVNVNGGAIALGHPIGASGCRILVTLLHEMQRRD |
| | | | AKKGIATLCVGGGMGVALAVERD* |

TABLE 55
List of Sequences

| | | SEQ ID |
|---|---|---|
| Description | Sequence | NO |
| Construct | ATGTCTCTACACTCTCCAGGTAAAGCGTTTCGCGCTGCACTTAGCAAA | SEQ ID |
| comprising a | GAAACCCCGTTGCAAATTGTTGGCACCATCAACGCTAACCATGCGCT | NO: |
| prpBCD gene | GCTGGCGCAGCGTGCCGGATATCAGGCGATTTATCTCTCCGGCGGTG | 138 |
| cassette; (as | GCGTGGCGGCAGGATCGCTGGGGCTGCCCGATCTCGGTATTTCTACT | |
| shown in | CTTGATGACGTGCTGACAGATATTCGCCGTATCACCGACGTTTGTTC | |
| FIG. 20) | GCTGCCGCTGCTGGTGGATGCGGATATCGGTTTTGGTTCTTCAGCCT | |
| ribosome | TTAACGTGGCGCGTACGGTGAAATCAATGATTAAAGCCGGTGCGGCA | |
| binding sites | GGATTGCATATTGAAGATCAGGTTGGTGCGAAACGCTGCGGTCATCG | |
| are | TCCGAATAAAGCGATCGTCTCGAAAGAAGAGATGGTGGATCGGATCC | |
| underlined; | GCGCGGCGGTGGATGCGAAAACCGATCCTGATTTTGTGATCATGGCG | |
| coding region | CGCACCGATGCGCTGGCGGTAGAGGGGCTGGATGCGGCGATCGAGC | |
| in bold | GTGCGCAGGCCTATGTTGAAGCGGGTGCCGAAATGCTGTTCCCGGAG | |
| | GCGATTACCGAACTCGCCATGTATCGCCAGTTTGCCGATGCGGTGCA | |
| | GGTGCCGATCCTCTCCAACATTACCGAATTTGGCGCAACACCGCTGT | |
| | TTACCACCGACGAATTACGCAGCGCCCATGTCGCAATGGCGCTCTAC | |
| | CCGCTTTCAGCGTTTCGCGCCATGAACCGCGCCGCTGAACATGTCTA | |
| | TAACATCCTGCGTCAGGAAGGCACACAGAAAAGCGTCATCGACACCA | |
| | TGCAGACCCGCAACGAGCTGTACGAAAGCATCAACTACTACCAGTAC | |
| | GAAGAGAAGCTCGACGACCTGTTTGCCCGTGGTCAGGTGAAATAA | |
| | AAACGCCCGTTGGTTGTATTCGACAACCGATGCCTGATGCGCCGCTGACG | |
| | CGACTTATCAGGCCTACGAGGTGAACTGAACTGTAGGTCGGATAAGACGC | |
| | ATAGCGTCGCATCCGACAACAATCTCGACCCTACAAATGATAACAATGAC | |
| | GAGGACAATATGAGCGACACAACGATCCTGCAAAACAGTACCCATGT | |
| | CATTAAACCGAAAAAATCGGTGGCACTTTCCGGCGTTCCGGCGGGCA | |
| | ATACGGCGCTCTGCACCGTGGGTAAAAGCGGCAACGACCTGCATTAC | |
| | CGTGGCTACGATATTCTTGATCTGGCGGAACATTGTGAATTTGAAGA | |
| | AGTGGCGCACCTGCTGATCCACGGCAAACTGCCAACCCGTGACGAAC | |
| | TCGCCGCCTACAAAACGAAACTGAAAGCCCTGCGTGGTTTACCGGCT | |
| | AACGTGCGTACCGTGCTGGAAGCCTTACCGGCGGCGTCACACCCGAT | |
| | GGATGTTATGCGCACCGGCGTTTCCGCGCTCGGCTGCACGCTGCCAG | |
| | AAAAAGAGGGGCACACCGTTTCTGGTGCGCGGGATATTGCCGACAAA | |
| | CTGCTGGCGTCACTTAGTTCGATTCTTCTCTACTGGTATCACTACAGC | |
| | CACAACGGCGAACGCATCCAGCCGGAAACTGATGACGACTCTATCGG | |
| | CGGTCACTTCCTGCATCTGCTGCACGGCGAAAAGCCGTCGCAAAGCT | |
| | GGGAAAAGGCGATGCATATCTCGCTGGTGCTGTACGCCGAACACGAG | |
| | TTTAACGCTTCCACCTTTACCAGCCGGGTGATTGCGGGCACTGGCTC | |
| | TGATATGTATTCCGCCATTATTGGCGCGATTGGCGCACTGCGCGGGC | |
| | CGAAACACGGCGGGGCGAATGAAGTGTCGCTGGAGATCCAGCAACG | |
| | CTACGAAACGCCGGGCGAAGCCGAAGCCGATATCCGCAAGCGGGTG | |
| | GAAAACAAAGAAGTGGTCATTGGTTTTGGGCATCCGGTTTATACCAT | |
| | CGCCGACCCGCGTCATCAGGTGATCAAACGTGTGGCGAAGCAGCTCT | |
| | CGCAGGAAGGCGGCTCGCTGAAGATGTACAACATCGCCGATCGCCTG | |
| | GAAACGGTGATGTGGGAGAGCAAAAAGATGTTCCCCAATCTCGACTG | |
| | GTTCTCCGCTGTTTCCTACAACATGATGGGTGTTCCCACCGAGATGTT | |
| | CACACCACTGTTTGTTATCGCCCGCGTCACTGGCTGGGCGGCGCACA | |
| | TTATCGAACAACGTCAGGACAACAAAATTATCCGTCCTTCCGCCAATT | |
| | ATGTTGGACCGGAAGACCGCCAGTTTGTCGCGCTGGATAAGCGCCAG | |
| | TAA | |
| | ACCTCTACGAATAACAATAAGGAAACGTACCCAATGTCAGCTCAAATCA | |
| | ACAACATCCGCCCGGAATTTGATCGTGAAATCGTTGATATCGTCGATT | |
| | ACGTGATGAACTACGAAATCAGCTCCAGAGTAGCCTACGACACCGCT | |
| | CATTACTGCCTGCTTGACACGCTCGGCTGCGGTCTGGAAGCTCTCGA | |
| | ATATCCGGCCTGTAAAAAACTGCTGGGGCCAATTGTCCCCGGCACCG | |
| | TCGTACCCAACGGCGTGCGCGTTCCCGGAACTCAGTTTCAGCTCGAC | |
| | CCCGTCCAGGCGGCATTTAACATTGGCGCGATGATCCGTTGGCTCGA | |
| | TTTCAACGATACCTGGCTGGCGGCGGAGTGGGGGCATCCTTCCGACA | |
| | ACCTCGGCGGCATTCTGGCAACGGCGGACTGGCTTTCGCGCAACGCG | |
| | ATCGCCAGCGGCAAAGCGCCGTTGACCATGAAACAGGTGCTGACCGG | |
| | AATGATCAAAGCCCATGAAATTCAGGGCTGCATCGCGCTGGAAAACT | |
| | CCTTTAACCGCGTTGGTCTCGACCACGTTCTGTTAGTGAAAGTGGCTT | |
| | CCACCGCCGTGGTCGCCGAAATGCTCGGCCTGACCCGCGAGGAAATT | |
| | CTCAACGCCGTTTCGCTGGCATGGGTAGACGGACAGTCGCTGCGCAC | |
| | TTATCGTCATGCACCGAACACCGGTACGCGTAAATCCTGGGCGGCGG | |
| | GCGATGCTACATCCCGCGCGGTACGTCTGGCGCTGATGGCGAAAACG | |
| | GGCGAAATGGGTTACCCGTCAGCCCTGACCGCGCCGGTGTGGGGTTT | |
| | CTACGACGTCTCCTTTAAAGGTGAGTCATTCCGCTTCCAGCGTCCGTA | |
| | CGGTTCCTACGTCATGGAAAATGTGCTGTTCAAAATCTCCTTCCCGGC | |
| | GGAGTTCCACTCCCAGACGGCAGTTGAAGCGGCGATGACGCTCTATG | |
| | AACAGATGCAGGCAGCAGGCAAAACGGCGGCAGATATCGAAAAAGT | |
| | GACCATTCGCACCCACGAAGCCTGTATTCGCATCATCGACAAAAAAG | |
| | GGCCGCTCAATAACCCGGCAGACCGCGACCACTGCATTCAGTACATG | |
| | GTGGCGATCCCGCTGCTGTTCGGACGCTTAACGGCGGCAGATTACGA | |
| | GGACAACGTTGCGCAAGATAAACGCATCGACGCCCTGCGCGAGAAGA | |
| | TCAATTGCTTTGAAGATCCGGCGTTTACCGCTGACTACCACGACCCG | |
| | GAAAAACGCGCCATCGCCAATGCCATAACCCTTGAGTTCACCGACGG | |
| | CACACGATTTGAAGAAGTGGTGGTGGAGTACCCAATTGGTCATGCTC | |
| | GCCGCCGTCAGGATGGCATTCCGAAGCTGGTCGATAAATTCAAAATC | |
| | AATCTCGCGCGCCAGTTCCCGACTCGCCAGCAGCAGCGCATTCTGGA | |
| | GGTTTCTCTCGACAGAACTCGCCTGGAACAGATGCCGGTCAATGAGT | |
| | ATCTCGACCTGTACGTCATTTAA | |
| Construct | GATCAAAAAGGTTAGCCTCAAGAGGGTCATAAAAATGTCAGAGCAGAAA | SEQ ID |
| comprising a | GTAGCTCTGGTTACCGGTGCGTTAGGTGGTATCGGAAGTGAGATCTGCCG | NO: |
| PhaBCA | CCAGCTTGTGACCGCCGGGTACAAGATTATCGCCACCGTTGTTCCACGCG | 139 |
| gene cassette; | AAGAAGACCGCGAAAAACAATGGTTGCAAAGTGAGGGGTTTCAAGACTC | |
| (as shown in | TGATGTGCGTTTCGTATTAACAGATTTAAACAATCACGAAGCTGCGACAG | |
| FIG. 11) | CGGCAATTCAAGAAGCGATTGCCGCCGAAGGACGCGTTGATGTATTGGTC | |
| ribosome | AACAACGCGGGGATCACGCGCGATGCTACATTTAAGAAAATGTCCTATGA | |
| binding sites | GCAATGGTCCCAAGTCATCGACACGAATTTAAAGACTCTTTTTACCGTGA | |
| are | CCCAGCCAGTATTTAATAAAATGCTTGAACAGAAGTCTGGCCGCATCGTA | |
| underlined | AACATTAGCTCTGTCAATGGTTTAAAAGGGCAATTTGGTCAAGCCAACTA | |
| | CTCGGCCTCGAAAGCAGGGATTATCGGGTTTACTAAAGCATTGGCGCAGG | |
| | AGGGTGCTCGCTCGAACATTTGCGTCAATGTCGTTGCTCCTGGTTACACAG | |
| | CGACACCCATGGTCACAGCAATGCGCGAGGATGTAATTAAGTCAATCGAA | |
| | GCTCAAATTCCCCTGCAACGTCTGGCAGCACCGGCGGAGATTGCGGCAGC | |
| | GGTTATGTATTTGGTGAGTGAACACGGTGCATACGTGACGGGCGAAACTT | |
| | TGAGTATCAACGGCGGGCTGTACATGCACTAAAGGTGCTTTTAGTCTAGC | |
| | GCTAGAGCAGGTACCATATTAATGAATCCAAATTCCTTTCAGTTTAAAGA | |
| | GAATATCTTACAGTTTTTCAGCGTGCACGACGATATTTGGAAAAAACTGC | |
| | AGGAATTTTACTATGGACAATCGCCCATCAATGAAGCGTTGGCGCAGTTA | |
| | AATAAGGAAGACATGAGTTTATTCTTCGAGGCGTTATCAAAAAACCCTGC | |
| | TCGTATGATGGAGATGCAGTGGTCCTGGTGGCAAGGGCAGATTCAAATTT | |
| | ACCAGAACGTGTTAATGCGTAGTGTAGCCAAGGACGTAGCCCCCTTTATC | |
| | CAGCCAGAGTCCGGAGATCGTCGCTTCAACTCGCCACTTTGGCAAGAACA | |
| | TCCAAATTTTGATTTACTGAGTCAATCCTACTTGTTGTTTTCTCAGTTGGTT | |
| | CAAAATATGGTGGATGTCGTTGAAGGAGTACCTGATAAGGTCCGCTATCG | |
| | CATCCATTTCTTTACACGTCAGATGATCAATGCGTTGTCTCCTTCTAATTTC | |
| | CTGTGGACGAACCCTGAAGTAATTCAACAGACGGTCGCTGAACAGGGTG | |
| | AGAATTTAGTACGCGGGATGCAAGTATTTCACGATGATGTAATGAATTCG | |
| | GGTAAATATTTGAGCATCCGTATGGTAAATAGCGACAGTTTCTCTCTTGGC | |
| | AAGGACTTGGCGTATACGCCAGGAGCCGTAGTTTTCGAGAACGACATCTT | |
| | TCAGCTTCTTCAATACGAAGCCACAACCGAGAACGTATATCAAACCCCTA | |
| | TTCTTGTCGTACCTCCCTTCATCAACAAGTACTACGTGCTGGACCTGCGCG | |
| | AACAGAATAGCTTGGTTAATTGGCTGCGCCAACAAGGACATACGGTGTTT | |
| | TTGATGTCGTGGCGTAACCCCAACGCAGAGCAGAAGGAGCTTACCTTCGC | |
| | TGACTTAATTACCCAAGGATCGGTAGAAGCATTACGTGTTATCGAAGAAA | |
| | TCACGGGAGAGAAAGAAGCTAACTGTATTGGATATTGCATCGGTGGTACA | |
| | CTTCTGGCTGCTACCCAGGCATATTATGTAGCTAAACGCCTGAAAAATCA | |
| | CGTAAAGTCAGCGACTTATATGGCGACGATTATTGATTTTGAGAACCCCG | |
| | GCTCATTGGGTGTTTTCATTAATGAGCCGGTCGTAAGTGGACTTGAAAAC | |
| | CTTAATAATCAACTTGGTTACTTCGACGGGCGTCAACTTGCAGTGACATTT | |
| | TCGTTGTTGCGCGAAAACACCTTGTATTGGAATTATTACATCGATAATTAC | |
| | TTGAAGGGTAAGGAACCGTCCGACTTTGACATCTTATACTGGAACTCGGA | |
| | TGGTACGAATATCCCAGCAAAGATTCACAATTTCCTGTTACGTAACCTTTA | |
| | TCTTAACAACGAACTTATTTCTCCAAATGCCGTCAAAGTTAATGGTGTGG | |
| | GTTTAAACCTTTCGCGCGTGAAGACTCCATCATTCTTCATTGCTACGCAGG | |
| | AGGACCATATCGCATTGTGGGATACCTGTTTTCGCGGCGCGGATTACCTG | |
| | GGGGGTGAGAGCACACTTGTGCTTGGGGAAAGCGGACACGTCGCCGGCA | |
| | TTGTCAACCCGCCTTCTCGTAACAAGTATGGTTGTTACACGAACGCCGCC | |
| | AAGTTTGAAAATACCAAGCAATGGCTTGACGGTGCAGAATATCATCCCGA | |
| | AAGCTGGTGGTTACGTTGGCAGGCATGGGTCACGCCTTATACTGGAGAGC | |
| | AGGTTCCTGCGCGTAATTTGGGAAACGCACAGTACCCCAGTATTGAAGCG | |
| | GCCCCTGGGCGTTATGTGCTGGTAAACCTGTTTTAACGCTCACATACAAG | |
| | CAATCTATAATTATTCACGGTATAAATGAAAGATGTTGTTATCGTAGCCG | |
| | CTAAACGCACTGCGATCGGTTCCTTTCTGGGGAGTCTGGCTTCCCTGAGCG | |
| | CCCCTCAGTTGGGTCAGACGGCTATCCGCGCAGTTTTGGATTCTGCAAAT | |
| | GTGAAACCAGAACAAGTGGACCAAGTAATTATGGGGAATGTGCTGACCA | |
| | CCGGCGTTGGGCAAAATCCTGCTCGTCAGGCAGCAATCGCCGCTGGGATT | |
| | CCTGTACAAGTTCCCGCCAGCACGCTTAATGTAGTGTGTGGGTCCGGATT | |
| | ACGTGCCGTTCACCTGGCAGCTCAAGCCATCCAATGCGATGAAGCCGATA | |
| | TCGTCGTTGCCGGAGGTCAAGAATCAATGTCCCAGTCTGCTCATTACATG | |
| | CAGCTTCGCAATGGCCAGAAAATGGGTAACGCACAGTTAGTCGATTCAAT | |
| | GGTGGCCGACGGCTTGACCGACGCGTATAATCAATACCAGATGGGTATCA | |
| | CCGCGGAGAATATCGTCGAAAAACTTGGTCTTAATCGTGAAGAACAAGAC | |
| | CAGCTTGCTCTGACAAGTCAACAACGTGCTGCAGCAGCGCAGGCTGCCGG | |
| | AAAATTCAAGGATGAAATTGCGGTCGTTTCGATTCCCCAGCGCAAAGGAG | |
| | AGCCGGTCGTCTTCGCGGAAGACGAATATATCAAGGCCAATACCTCGTTG | |
| | GAATCCTTGACGAAACTGCGTCCAGCATTCAAAAAAGACGGTTCTGTTAC | |
| | AGCCGGCAACGCATCTGGCATTAATGATGGGGCAGCCGCGGTCCTGATGA | |
| | TGTCCGCCGACAAAGCGGCTGAACTGGGCTTAAAGCCTTTAGCACGCATT | |
| | AAAGGTTACGCGATGTCAGGAATTGAGCCGGAAATCATGGGACTGGGTC | |
| | CTGTAGACGCCGTTAAGAAAACCCTTAATAAGGCTGGTTGGTCCTTAGAC | |
| | CAGGTCGATCTGATCGAGGCCAATGAGGCTTTTGCTGCCCAAGCACTGGG | |
| | AGTAGCCAAGGAGCTTGGGCTGGACCTGGACAAGGTAAATGTTAACGGA | |
| | GGTGCGATCGCGCTGGGACACCCGATCGGGGCTTCGGGTTGTCGTATCTT | |
| | GGTCACGTTATTACACGAAATGCAGCGTCGTGATGCAAAGAAGGGTATCG | |
| | CCACATTGTGTGTGGGAGGTGGAATGGGGGTGGCGCTTGCCGTTGAGCGC | |
| | GATTAA | |
| MatB | MNANLFARLFDKLDDPHKLAIETAAGDKISYAELVARAGRVANVLVARGLQ | 140 |
| (methylmalonyl- | VGDRVAAQTEKSVEALVLYLATVRAGGVYLPLNTAYTLHELDYFITDAEPKI | |
| coa | VVCDPSKRDGIAAIAAKVGATVETLGPDGRGSLTDAAAGASEAFATIDRGAD | |
| synthetase) | DLAAILYTSGTTGRSKGAMLSHDNLASNSLTLVDYWRFTPDDVLIHALPIYH | |
| Rhodopseudomonas | THGLFVASNVTLFARGSMIFLPKFDPDKILDLMARATVLMGVPTFYTRLLQSP | |
| palustris | RLTKETTGHMRLFISGSAPLLADTHREWSAKTGHAVLERYGMTETNMNTSN | |
| (polypeptide) | PYDGDRVPGAVGPALPGVSARVTDPETGKELPRGDIGMIEVKGPNVFKGYW | |
| | RMPEKTKSEFRDDGFFITGDLGKIDERGYVHILGRGKDLVITGGFNVYPKEIES | |
| | EIDAMPGVVESAVIGVPHADFGEGVTAVVVRDKGATIDEAQVLHGLDGQLA | |
| | KFKMPKKVIFVDDLPRNTMGKVQKNVLRETYKDIYK | |
| MatB | ATGAATGCAAATCTGTTTGCTCGTCTGTTCGACAAATTAGACGATCCACATAAGTTA | 141 |
| (methylmalonyl- | GCCATTGAAACTGCTGCAGGTGATAAGATTTCGTATGCAGAGCTTGTTGCCCGCGCA | |
| coa | GGTCGCGTCGCAAATGTACTTGTAGCCCGCGGACTGCAGGTAGGAGATCGTGTAGCT | |
| synthetase) | GCTCAGACAGAGAAATCTGTAGAAGCGTTGGTTTTATATTTAGCAACTGTGCGTGCT | |
| Rhodopseudomonas | GGGGGGGTATACCTTCCACTGAACACCGCATATACTTTACATGAATTAGATTACTTC | |
| palustris | ATCACAGACGCCGAGCCGAAAATTGTTGTCTGCGATCCATCGAAGCGCGACGGGATC | |
| (codon | GCTGCCATTGCAGCAAAGGTAGGCGCGACAGTCGAAACTCTTGGACCGGATGGCCGT | |
| optimized for | GGCTCTCTTACTGACGCCGCTGCGGGAGCCTCAGAAGCCTTTGCAACTATTGATCGC | |
| expression in | GGCGCCGACGATCTGGCGGCTATCCTTTATACCAGCGGGACCACGGGGCGTAGCAAG | |
| E. coli | GGTGCGATGCTTTCGCACGACAATCTGGCAAGCAACTCGCTTACACTGGTGGATTAC | |
| | TGGCGCTTCACACCGGACGACGTGTTGATTCATGCATTGCCAATTTACCACACGCAC | |
| | GGATTATTTGTCGCATCCAATGTGACTTTATTCGCGCGCGGGTCGATGATTTTCTTA | |
| | CCCAAATTCGATCCGGATAAGATTTTAGACCTTATGGCTCGTGCAACGGTTTTAATG | |
| | GGCGTACCGACTTTCTACACTCGCCTGCTTCAGAGCCCGCGCTTGACGAAGGAGACA | |
| | ACGGGTCACATGCGCTTATTCATTAGCGGCAGTGCCCCCCTGTTGGCAGACACTCAC | |
| | CGTGAATGGTCCGCTAAAACCGGACACGCAGTTTTAGAACGTTATGGGATGACGGAG | |
| | ACAAACATGAACACGAGCAATCCATATGATGGTGACCGTGTACCGGGGGCCGTCGGT | |
| | CCCGCATTACCAGGGGTATCTGCTCGCGTCACTGATCCGGAAACTGGAAAAGAGCTG | |
| | CCGCGTGGTGACATCGGAATGATTGAAGTTAAAGGACCCAACGTATTCAAAGGATAT | |
| | TGGCGTATGCCGGAAAAGACTAAGTCGGAGTTTCGCGACGATGGTTTCTTCATTACA | |
| | GGAGATTTGGGGAAAATCGATGAACGTGGGTATGTTCACATTCTTGGGCGCGGTAAG | |
| | GATCTTGTGATCACCGGTGGCTTTAACGTCTATCCAAAAGAAATTGAATCAGAGATC | |
| | GACGCCATGCCAGGGGTAGTGGAATCTGCGGTAATTGGCGTGCCCCATGCGGATTTT | |
| | GGTGAAGGCGTCACCGCCGTCGTTGTACGCGATAAAGGAGCCACGATCGATGAAGCC | |
| | CAGGTACTTCATGGACTGGACGGACAGTTAGCCAAGTTTAAGATGCCGAAGAAGGTA | |
| | ATCTTTGTGGACGATCTTCCTCGTAACACAATGGGTAAGGTACAAAAAAACGTTCTG | |
| | CGCGAGACTTACAAAGACATTTATAAA | |

TABLE 56
Inducible promoter construct sequences

| | | SEQ |
|---|---|---|
| | | ID |
| Description | Sequence | NO |
| Arabinose | CAGACATTGCCGTCACTGCGTCTTTTACTGGCTCTTCTCGCTAACCCAACCG | 142 |
| Promoter | GTAACCCCGCTTATTAAAAGCATTCTGTAACAAAGCGGGACCAAAGCCATG | |
| region | ACAAAAACGCGTAACAAAAGTGTCTATAATCACGGCAGAAAAGTCCACATT | |
| | GATTATTTGCACGGCGTCACACTTTGCTATGCCATAGCATTTTTATCCATAA | |
| | GATTAGCGGATCCAGCCTGACGCTTTTTTTCGCAACTCTCTACTGTTTCTCCA | |
| | TACCTCTAGAAATAATTTTGTTTAACTTTAAGAAGGAGATATACAT | |
| AraC (reverse | TTATTCACAACCTGCCCTAAACTCGCTCGGACTCGCCCCGGTGCATTTTTTA | 143 |
| orientation) | AATACTCGCGAGAAATAGAGTTGATCGTCAAAACCGACATTGCGACCGACG | |
| | GTGGCGATAGGCATCCGGGTGGTGCTCAAAAGCAGCTTCGCCTGACTGATG | |
| | CGCTGGTCCTCGCGCCAGCTTAATACGCTAATCCCTAACTGCTGGCGGAACA | |
| | AATGCGACAGACGCGACGGCGACAGGCAGACATGCTGTGCGACGCTGGCG | |
| | ATATCAAAATTACTGTCTGCCAGGTGATCGCTGATGTACTGACAAGCCTCGC | |
| | GTACCCGATTATCCATCGGTGGATGGAGCGACTCGTTAATCGCTTCCATGCG | |
| | CCGCAGTAACAATTGCTCAAGCAGATTTATCGCCAGCAATTCCGAATAGCG | |
| | CCCTTCCCCTTGTCCGGCATTAATGATTTGCCCAAACAGGTCGCTGAAATGC | |
| | GGCTGGTGCGCTTCATCCGGGCGAAAGAAACCGGTATTGGCAAATATCGAC | |
| | GGCCAGTTAAGCCATTCATGCCAGTAGGCGCGCGGACGAAAGTAAACCCAC | |
| | TGGTGATACCATTCGTGAGCCTCCGGATGACGACCGTAGTGATGAATCTCTC | |
| | CAGGCGGGAACAGCAAAATATCACCCGGTCGGCAGACAAATTCTCGTCCCT | |
| | GATTTTTCACCACCCCCTGACCGCGAATGGTGAGATTGAGAATATAACCTTT | |
| | CATTCCCAGCGGTCGGTCGATAAAAAAATCGAGATAACCGTTGGCCTCAAT | |
| | CGGCGTTAAACCCGCCACCAGATGGGCGTTAAACGAGTATCCCGGCAGCAG | |
| | GGGATCATTTTGCGCTTCAGCCATACTTTTCATACTCCCGCCATTCAGAGAA | |
| | GAAACCAATTGTCCATATTGCAT | |
| AraC | MQYGQLVSSLNGGSMKSMAEAQNDPLLPGYSFNAHLVAGLTPIEANGYLDFFI | 144 |
| polypeptide | DRPLGMKGYILNLTIRGQGVVKNQGREFVCRPGDILLFPPGEIHHYGRHPEAHE | |
| | WYHQWVYFRPRAYWHEWLNWPSIFANTGFFRPDEAHQPHFSDLFGQIINAGQ | |
| | GEGRYSELLAINLLEQLLLRRMEAINESLHPPMDNRVREACQYISDHLADSNFDI | |
| | ASVAQHVCLSPSRLSHLFRQQLGISVLSWREDQRISQAKLLLSTTRMPIATVGR | |
| | NVGFDDQLYFSRVFKKCTGASPSEFRAGCE* | |
| Region | CGGTGAGCATCACATCACCACAATTCAGCAAATTGTGAACATCATCACGTT | 145 |
| comprising | CATCTTTCCCTGGTTGCCAATGGCCCATTTTCCTGTCAGTAACGAGAAGGTC | |
| rhamnose | GCGAATCAGGCGCTTTTTAGACTGGTCGTAATGAAATTCAGCTGTCACCGG | |
| inducible | ATGTGCTTTCCGGTCTGATGAGTCCGTGAGGACGAAACAGCCTCTACAAAT | |
| promoter | AATTTTGTTTAAAACAACACCCACTAAGATAACTCTAGAAATAATTTTGTTT | |
| | AACTTTAAGAAGGAGATATACAT | |
| Lac Promoter | ATTCACCACCCTGAATTGACTCTCTTCCGGGCGCTATCATGCCATACCGCGA | 146 |
| region | AAGGTTTTGCGCCATTCGATGGCGCGCCGCTTCGTCAGGCCACATAGCTTTC | |
| | TTGTTCTGATCGGAACGATCGTTGGCTGTGTTGACAATTAATCATCGGCTCG | |
| | TATAATGTGTGGAATTGTGAGCGCTCACAATTAGCTGTCACCGGATGTGCTT | |
| | TCCGGTCTGATGAGTCCGTGAGGACGAAACAGCCTCTACAAATAATTTTGTT | |
| | TAAAACAACACCCACTAAGATAACTCTAGAAATAATTTTGTTTAACTTTAAG | |
| | AAGGAGATATACAT | |
| LacO | GGAATTGTGAGCGCTCACAATT | 147 |
| LacI (in | TCACTGCCCGCTTTCCAGTCGGGAAACCTGTCGTGCCAGCTGCATTAATGAA | 148 |
| reverse | TCGGCCAACGCGCGGGGAGAGGCGGTTTGCGTATTGGGCGCCAGGGTGGTT | |
| orientation) | TTTCTTTTCACCAGTGAGACTGGCAACAGCTGATTGCCCTTCACCGCCTGGC | |
| | CCTGAGAGAGTTGCAGCAAGCGGTCCACGCTGGTTTGCCCCAGCAGGCGAA | |
| | AATCCTGTTTGATGGTGGTTAACGGCGGGATATAACATGAGCTATCTTCGGT | |
| | ATCGTCGTATCCCACTACCGAGATATCCGCACCAACGCGCAGCCCGGACTC | |
| | GGTAATGGCGCGCATTGCGCCCAGCGCCATCTGATCGTTGGCAACCAGCAT | |
| | CGCAGTGGGAACGATGCCCTCATTCAGCATTTGCATGGTTTGTTGAAAACCG | |
| | GACATGGCACTCCAGTCGCCTTCCCGTTCCGCTATCGGCTGAATTTGATTGC | |
| | GAGTGAGATATTTATGCCAGCCAGCCAGACGCAGACGCGCCGAGACAGAA | |
| | CTTAATGGGCCCGCTAACAGCGCGATTTGCTGGTGACCCAATGCGACCAGA | |
| | TGCTCCACGCCCAGTCGCGTACCGTCCTCATGGGAGAAAATAATACTGTTG | |
| | ATGGGTGTCTGGTCAGAGACATCAAGAAATAACGCCGGAACATTAGTGCAG | |
| | GCAGCTTCCACAGCAATGGCATCCTGGTCATCCAGCGGATAGTTAATGATC | |
| | AGCCCACTGACGCGTTGCGCGAGAAGATTGTGCACCGCCGCTTTACAGGCT | |
| | TCGACGCCGCTTCGTTCTACCATCGACACCACCACGCTGGCACCCAGTTGAT | |
| | CGGCGCGAGATTTAATCGCCGCGACAATTTGCGACGGCGCGTGCAGGGCCA | |
| | GACTGGAGGTGGCAACGCCAATCAGCAACGACTGTTTGCCCGCCAGTTGTT | |
| | GTGCCACGCGGTTGGGAATGTAATTCAGCTCCGCCATCGCCGCTTCCACTTT | |
| | TTCCCGCGTTTTCGCAGAAACGTGGCTGGCCTGGTTCACCACGCGGGAAAC | |
| | GGTCTGATAAGAGACACCGGCATACTCTGCGACATCGTATAACGTTACTGG | |
| | TTTCAT | |
| LacI | MKPVTLYDVAEYAGVSYQTVSRVVNQASHVSAKTREKVEAAMAELNYIPNRV | 149 |
| polypeptide | AQQLAGKQSLLIGVATSSLALHAPSQIVAAIKSRADQLGASVVVSMVERSGVE | |
| sequence | ACKAAVHNLLAQRVSGLIINYPLDDQDAIAVEAACTNVPALFLDVSDQTPINSII | |
| | FSHEDGTRLGVEHLVALGHQQIALLAGPLSSVSARLRLAGWHKYLTRNQIQPIA | |
| | EREGDWSAMSGFQQTMQMLNEGIVPTAMLVANDQMALGAMRAITESGLRVG | |
| | ADISVVGYDDTEDSSCYIPPLTTIKQDFRLLGQTSVDRLLQLSQGQAVKGNQLL | |
| | PVSLVKRKTTLAPNTQTASPRALADSLMQLARQVSRLESGQ | |
| Region | ACGTTAAATCTATCACCGCAAGGGATAAATATCTAACACCGTGCGTGTTGA | 150 |
| comprising | CTATTTTACCTCTGGCGGTGATAATGGTTGCATAGCTGTCACCGGATGTGCT | |
| Temperature | TTCCGGTCTGATGAGTCCGTGAGGACGAAACAGCCTCTACAAATAATTTTGT | |
| sensitive | TTAAAACAACACCCACTAAGATAACTCTAGAAATAATTTTGTTTAACTTTAA | |
| promoter | GAAGGAGATATACAT | |
| mutant cI857 | TCAGCCAAACGTCTCTTCAGGCCACTGACTAGCGATAACTTTCCCCACAACG | 151 |
| repressor | GAACAACTCTCATTGCATGGGATCATTGGGTACTGTGGGTTTAGTGGTTGTA | |
| | AAAACACCTGACCGCTATCCCTGATCAGTTTCTTGAAGGTAAACTCATCACC | |
| | CCCAAGTCTGGCTATGCAGAAATCACCTGGCTCAACAGCCTGCTCAGGGTC | |
| | AACGAGAATTAACATTCCGTCAGGAAAGCTTGGCTTGGAGCCTGTTGGTGC | |
| | GGTCATGGAATTACCTTCAACCTCAAGCCAGAATGCAGAATCACTGGCTTTT | |
| | TTGGTTGTGCTTACCCATCTCTCCGCATCACCTTTGGTAAAGGTTCTAAGCTT | |
| | AGGTGAGAACATCCCTGCCTGAACATGAGAAAAAACAGGGTACTCATACTC | |
| | ACTTCTAAGTGACGGCTGCATACTAACCGCTTCATACATCTCGTAGATTTCT | |
| | CTGGCGATTGAAGGGCTAAATTCTTCAACGCTAACTTTGAGAATTTTTGTAA | |
| | GCAATGCGGCGTTATAAGCATTTAATGCATTGATGCCATTAAATAAAGCAC | |
| | CAACGCCTGACTGCCCCATCCCCATCTTGTCTGCGACAGATTCCTGGGATAA | |
| | GCCAAGTTCATTTTTCTTTTTTTCATAAATTGCTTTAAGGCGACGTGCGTCCT | |
| | CAAGCTGCTCTTGTGTTAATGGTTTCTTTTTTGTGCTCAT | |
| RBS and | CTCTAGAAATAATTTTGTTTAACTTTAAGAAGGAGATATACAT | [[104]] |
| leader region | | 318 |
| mutant cI857 | MSTKKKPLTQEQLEDARRLKAIYEKKKNELGLSQESVADKMGMGQSGVGALF | [[105]] |
| repressor | NGINALNAYNAALLTKILKVSVEEFSPSIAREIYEMYEAVSMQPSLRSEYEYPVF | 319 |
| polypeptide | SHVQAGMFSPKLRTFTKGDAERWVSTTKKASDSAFWLEVEGNSMTAPTGSKP | |
| sequence | SFPDGMLILVDPEQAVEPGDFCIARLGGDEFTFKKLIRDSGQVFLQPLNPQYPMI | |
| | PCNESCSVVGKVIASQWPEETFG | |
| TetR-tet | Ttaagacccactttcacatttaagttgtttttctaatccgcatatgatcaattcaagg | [[106]] |
| promoter | ccgaataagaaggctggctctgcaccttggtgatcaaataattcgatagcttg | 320 |
| construct | tcgtaataatggcggcatactatcagtagtaggtgtttccctttcttctttagcgacttg | |
| | atgctcttgatcttccaatacgcaacctaaagtaaaatgccccacagcgctgagtgcatataat | |
| | gcattctctagtgaaaaaccttgttggcataaaaaggctaattgattttcgagagtttcatactgt | |
| | ttttctgtaggccgtgtacctaaatgtacttttgctccatcgcgatgacttagtaaagcacatctaaa | |
| | acttttagcgttattacgtaaaaaatcttgccagctttccccttctaaagggcaaaagtgagtatggt | |
| | gcctatctaacatctcaatggctaaggcgtcgagcaaagcccgcttattttttacatgccaatacaa | |
| | tgtaggctgctctacacctagcttctgggcgagtttacgggttgttaaaccttcgattccgacctca | |
| | ttaagcagctctaatgcgctgttaatcactttacttttatctaatctagacatcattaattcctaa | |
| | tttttgttgacactctatcattgatagagttattttaccactccctatcagtgatagagaaaagtgaa | |
| | ctctagaaataattttgtttaactttaagaaggagatatacat | |
| PssB | TCACCTTTCCCGGATTAAACGCTTTTTTGCCCGGTGGCATGGTGCTAC | [[107]]321 |
| promoter | CGGCGATCACAAACGGTTAATTATGACACAAATTGACCTGAATGAA | |
| | TATACAGTATTGGAATGCATTACCCGGAGTGTTGTGTAACAATGTCT | |
| | GGCCAGGTTTGTTTCCCGGAACCGAGGTCACAACATAGTAAAAGCG | |
| | CTATTGGTAATGGTACAATCGCGCGTTTACACTTATTC | |

1. A bacterium comprising gene sequence(s) encoding one or more propionate catabolism enzyme(s) operably linked to a directly or indirectly inducible promoter that is not associated with the propionate catabolism enzyme gene in nature.

2. The bacterium of claim 1, wherein the bacterium further comprises gene sequence(s) encoding one or more transporter(s) of propionate operably linked to a promoter that is not associated with the transporter gene in nature.

3. The bacterium of claim 1, wherein the bacterium further comprises gene sequence(s) encoding one or more exporter(s) of succinate operably linked to a promoter that is not associated with the transporter gene in nature.

4. The bacterium of claim 1, wherein the bacterium further comprises a genetic modification that reduces the import of succinate into the bacterium.

5. The bacterium claim 2, wherein the promoter is a directly or indirectly inducible promoter.

6. The bacterium of claim 1, wherein the bacterium further comprises a genetic modification that reduces endogenous biosynthesis of propionate in the bacterium.

7. The bacterium of claim 2, wherein the promoter operably linked to the gene sequence(s) encoding a propionate catabolism enzyme and the promoter operably linked to the gene sequence(s) encoding a transporter of propionate are separate copies of the same promoter.

8. The bacterium of claim 2, wherein the promoter operably linked to the gene sequence(s) encoding a propionate catabolism enzyme and the promoter operably linked to the gene sequence(s) encoding a transporter of propionate are the same copy of the same promoter.

9. The bacterium of claim 2, wherein the promoter operably linked to the gene sequence(s) encoding a propionate catabolism enzyme and the promoter operably linked to the gene sequence(s) encoding a transporter of propionate are different promoters.

10. The bacterium of claim 1, wherein the promoter operably linked to the gene sequence(s) encoding a propionate catabolism enzyme is directly or indirectly induced by exogenous environmental conditions found in the mammalian gut.

11. The bacterium of claim 1, wherein the promoter operably linked to the gene sequence(s) encoding a propionate catabolism enzyme is directly or indirectly induced under low-oxygen or anaerobic conditions.

12. The bacterium of claim 1, wherein the promoter operably linked to the gene sequence(s) encoding a propionate catabolism enzyme is selected from the group consisting of an FNR-responsive promoter, an ANR-responsive promoter, and a DNR-responsive promoter.

13. The bacterium of claim 1, wherein the promoter operably linked to the gene sequence(s) encoding a propionate catabolism enzyme is an FNRS promoter.

14. The bacterium of claim 2, wherein the promoter operably linked to the gene sequence(s) encoding a transporter of propionate is directly or indirectly induced by exogenous environmental conditions found in the mammalian gut.

15. The bacterium of claim 2, wherein the promoter operably linked to the gene sequence(s) encoding a transporter of propionate is directly or indirectly induced under low-oxygen or anaerobic conditions.

16. The bacterium of claim 2, wherein the promoter operably linked to the gene sequence(s) encoding a transporter of propionate is selected from the group consisting of an FNR-responsive promoter, an ANR-responsive promoter, and a DNR-responsive promoter.

17. The bacterium of claim 1, wherein the gene sequence(s) encoding a propionate catabolism enzyme is located on a chromosome in the bacterium.

18. The bacterium of claim 1, wherein the gene sequence(s) encoding a propionate catabolism enzyme is located on a plasmid in the bacterium.

19. The bacterium of claim 1, wherein the bacterium comprises gene sequence(s) encoding one or more propionate catabolism enzyme(s) that convert propionate to succinate.

20. The bacterium of claim 1, wherein the bacterium comprises gene sequence(s) encoding one or more propionate catabolism enzyme(s) selected from prpE, pccB, accA1, mmcE, mutA, and mutB.

21. The bacterium of claim 1, wherein the gene sequence(s) encoding one or more propionate catabolism enzyme(s) are present in a single gene cassette.

22. The bacterium of claim 1, wherein the bacterium comprises at least two gene sequence(s) encoding one or more propionate catabolism enzyme(s) and wherein the gene sequences are present in two or more separate gene cassettes.

23. The bacterium of claim 22, wherein the gene sequence(s) encoding one or more propionate catabolism enzyme(s) are present in a first gene cassette, operably linked to a first promoter and present in a second gene cassette, operably linked to a second promoter.

24. The bacterium of claim 23, wherein the first promoter and the second promoter are inducible promoters.

25. The bacterium of claim 23, wherein the first promoter and the second promoter are different promoters.

26. The bacterium of claim 23, wherein the first promoter and the second promoter are separate copies of the same promoter.

27. The bacterium of claim 23, wherein the first gene cassette comprises prpE, pccB, and accA1 and the second gene cassette comprises mmcE, mutA, and mutB.

28. The bacterium of claim 27, wherein the gene sequence(s) encoding prpE has at least 90% identity to SEQ ID NO: 25.

29. The bacterium of claim 27, wherein the gene sequence(s) encoding pccB has at least 90% identity to SEQ ID NO: 39.

30. The bacterium of claim 27, wherein the gene sequence(s) encoding accA1 has at least 90% identity to SEQ ID NO: 38.

31. The bacterium of claim 27, wherein the gene sequence(s) encoding mmcE has at least 90% identity to SEQ ID NO: 32.

32. The bacterium of claim 27, wherein the gene sequence(s) encoding mutA has at least 90% identity to SEQ ID NO: 33.

33. The bacterium of claim 27, wherein the gene sequence(s) encoding mutB has at least 90% identity to SEQ ID NO: 34.

34. The bacterium of claim 1, wherein the bacterium comprises one or more gene sequence(s) encoding one or more propionate catabolism enzyme(s) that convert propionate to polyhydroxyalkanoate.

35. The bacterium of claim 34, wherein the bacterium comprises one or more gene sequence(s) encoding prpE, phaB, phaC, and phaA.

36. The bacterium of claim 35, wherein the gene sequence(s) encoding prpE has at least 90% identity to SEQ ID NO: 25.

37. The bacterium of claim 35, wherein the gene sequence(s) encoding phaB has at least 90% identity to a sequence encoding SEQ ID NO: 26.

38. The bacterium of claim 35, wherein the gene sequence(s) encoding phaC has at least 90% identity to a sequence encoding SEQ ID NO: 27.

39. The bacterium of claim 35, wherein the gene sequence(s) encoding phaA has at least 90% identity to a sequence encoding SEQ ID NO: 28.

40. The bacterium of claim 1, wherein the bacterium comprises gene sequence(s) encoding one or more propionate catabolism enzyme(s) that convert propionate to pyruvate and succinate.

41. The bacterium of claim 40, wherein the one or more gene sequence(s) encode prpB, a prpC, and prpD.

42. The bacterium of claim 40, wherein the one or more gene sequence(s) encode prpE.

43. The bacterium of claim 40, wherein the gene sequence(s) encoding prpE has at least 90% identity to SEQ ID NO: 25.

44. The bacterium of claim 40, wherein the one or more gene sequence(s) encoding prpC has at least 90% identity to SEQ ID NO: 57.

45. The bacterium of claim 40, wherein the one or more gene sequence(s) encoding prpD has at least 90% identity to SEQ ID NO: 58.

46. The bacterium of claim 40, wherein the one or more gene sequence(s) encoding prpB has at least 90% identity to SEQ ID NO: 56.

47. The bacterium of claim 1, wherein the one or more gene sequence(s) encoding one or more propionate catabolism enzyme(s) comprise one or more gene(s) encoding one or more propionate catabolism enzyme(s) located on a plasmid in the bacterial cell.

48. The bacterium of claim 1, wherein the one or more gene sequence(s) encoding one or more propionate catabolism enzyme(s) comprise one or more gene(s) encoding one or more propionate catabolism enzyme(s) located on a chromosome in the bacterial cell.

49. The bacterium of claim 3, wherein the gene sequence(s) encoding the succinate exporter encodes dcuC.

50. The bacterium of claim 49, wherein the gene sequence(s) encoding dcuC is at least about 90% identity to the sequence of SEQ ID NO: 49.

51. The bacterium of claim 3, wherein the gene sequence(s) encoding the succinate exporter encodes sucE1.

52. The bacterium of claim 51, wherein the gene sequence(s) encoding sucE1 has at least about 90% identity to the sequence of SEQ ID NO: 46.

53. The bacterium of claim 1, wherein the engineered bacterial cell further comprises a genetic modification that increases activity of the at least one heterologous gene encoding the at least one propionate catabolism enzyme.

54. The bacterium of claim 1, wherein the engineered bacterial cell further comprises a genetic modification that increases activity of prpE.

55. The bacterium of claim 1, wherein the engineered bacterial cell further comprises a genetic modification in pka.

56. The bacterium of claim 1, wherein the bacterium is a probiotic bacterial cell.

57. The bacterium of claim 1, wherein the bacterium is a member of a genus selected from the group consisting of Bacteroides, Bifidobacterium, Clostridium, Escherichia, Lactobacillus and Lactococcus.

58. The bacterium of claim 1, wherein the bacterium is of the genus Escherichia.

59. The bacterium of claim 1, wherein the engineered bacterial cell is of the species Escherichia coli strain Nissle.

60. The bacterium of claim 1, wherein the engineered bacterial cell is an auxotroph in a gene that is complemented when the engineered bacterial cell is present in a mammalian gut.

61. The bacterium of claim 60, wherein the mammalian gut is a human gut.

62. The bacterium of claim 60, wherein the engineered bacterial cell is an auxotroph in diaminopimelic acid or an enzyme in the thymine biosynthetic pathway.

63. The bacterium of claim 1, wherein the engineered bacterial cell is further engineered to harbor a gene encoding a substance that is toxic to the bacterium, wherein the gene is under the control of a promoter is directly or indirectly induced by an environmental condition not naturally present in the mammalian gut.

64. A pharmaceutical composition comprising the bacterium in claim 1, and a pharmaceutically acceptable carrier.

65. The pharmaceutical composition of claim 64 formulated for oral administration.

66. A method for reducing the levels of propionate, methylmalonate and their byproduct molecules in a subject and/or treating a disease or disorder involving the catabolism of propionate in a subject, the method comprising administering a pharmaceutical composition of claim 64.

67. The method of claim 66, wherein the disorder involving the catabolism of propionate is an organic acidemia.

68. The method of claim 67, wherein the organic acidemia is propionic acidemia (PA).

69. The method of claim 67, wherein the organic acidemia is methylmalonic acidemia (MMA).

70. The method of claim 66, wherein the disorder involving the catabolism of propionate is a vitamin B₁₂ deficiency.
